(19) United States
(12) Patent Application Publication
(10) Pub. No.: US 2025/0333406 A1
(43) Pub. Date: Oct. 30, 2025

(54) SULFONYL CYCLIC DERIVATIVES, AND COMPOSITIONS AND METHODS THEREOF

(71) Applicant: Lysoway Therapeutics, Inc., Cambridge, MA (US)

(72) Inventors: Yongchang Qiu, Acton, MA (US); Xiang Y. Yu, Acton, MA (US); Christopher John Swain, Duxford (GB)

(21) Appl. No.: 18/868,450

(22) PCT Filed: May 30, 2023
(86) PCT No.: PCT/US23/23849

§ 371 Date: Nov. 22, 2024

(51) Int. Cl. C07D 417/14; C07D 401/14; C07D 403/04; C07D 405/14; C07D 413/14

(52) U.S. Cl. CPC C07D 417/14; C07D 401/14; C07D 403/04; C07D 405/14; C07D 413/14

(57) ABSTRACT

The invention provides novel sulfonyl cyclic derivatives, and compositions and methods of preparation and use thereof, that are useful in treating various diseases and disorders related to TRPML activities such as lysosome storage diseases, muscular dystrophy, neurodegenerative diseases, oxidative stress or reactive oxygen species (ROS) related diseases, metabolic diseases, metastatic cancer, and ageing.

### PRIORITY CLAIMS AND RELATED PATENT APPLICATIONS

This application claims the benefit of priority from U.S. Provisional Application Ser. No. 63/347,557, filed on May 31, 2022, the entire content of which is incorporated herein by reference in its entirety.

### TECHNICAL FIELD OF THE INVENTION

The invention generally relates to novel compounds and therapeutic uses thereof. More particularly, the invention provides novel sulfonyl cyclic derivatives, their salts, solvates, hydrates and polymorphs thereof as transient receptor potential cation channel, mucolipin subfamily (TRPML) modulators. The invention also provides pharmaceutical compositions comprising a compound of the invention and methods thereof for treating various diseases and disorders associated with or related to TRPML activities such as lysosome storage diseases, muscular dystrophy, neurodegenerative diseases, reactive oxygen species (ROS) or oxidative stress related diseases, metabolic diseases, metastatic cancer, and ageing.

### BACKGROUND OF THE INVENTION

The lysosome, the cell's recycling center, can mediate the degradation of a variety of biomaterials (proteins, lipids, and membranes) into smaller molecules or building blocks, which will be subsequently transported out of lysosomes for reutilization or energy (see, e.g., de Duve 2005 Nat Cell Biol 7(9): 847-9; Parkinson-Lawrence, et al. 2010 Physiology (Bethesda) 25(2): 102-15). Problems in either the degradation step (due to lack of hydrolytic enzymes) or the transport step lead to lysosome storage (of accumulated materials) and more than 50 human diseases collectively called lysosome storage diseases (LSDs). Lysosome storage can in turn affect lysosomal degradation and membrane transport/trafficking, making a positive feedback loop and a vicious cycle. Because lysosome storage is also seen in common neurodegenerative diseases such as Alzheimer's and Parkinson's, understanding the mechanisms underlying the positive feedback loop may provide therapeutic approaches not only for LSDs, but also for common sporadic neurodegenerative diseases. A lysosome-localized Ca²⁺ channel, TRPML1, has been recently identified as a key regulator of most membrane trafficking processes in the lysosome. Human mutations of TRPML1 cause lysosomal trafficking defects, lysosome storage, and neurodegenerative diseases.

TRPML1 (also abbreviated as ML1), a member of the TRP-type Ca²⁺ channel superfamily, is the principle Ca²⁺ channel in the lysosome (see, e.g., Cheng, et al. 2010 FEBS Lett 584(10): 2013-21). Loss-of-function mutations in the human TRPML1 gene cause Type IV Mucolipidosis (ML4), a lysosome storage neurodegenerative disease. TRPML1^{-/-} (abbreviated as ML1^{-/-}) skin fibroblasts from ML4 patients are characterized by the accumulation of enlarged endosomal/lysosomal compartments (vacuoles) in which lipids and other biomaterials build up, suggestive of trafficking defects. Analyses of trafficking kinetics suggest that the primary defects are in the late endocytic pathways. First, ML1 is likely to be required for the formation of transport vesicles from the LEL to the Trans-Golgi Network (TGN) (LEL-to-TGN retrograde trafficking). Second, fusion of lysosomes with the plasma membrane (referred to as lysosomal exocytosis), a process that is important in cellular waste elimination, membrane repair, and phagocytosis, is defective in ML4 cells. Defects in either trafficking steps could lead to lysosome storage. Because the release of Ca²⁺ from lysosomes (lysosomal Ca²⁺ release) is essential for both trafficking steps, it is hypothesized that ML1 is indeed the Ca²⁺ release channel that regulates lysosomal trafficking

PI(3,5)P₂, a low-abundance phosphoinositide, is the primary activator of ML1 and positive regulator of lysosomal trafficking. Both TRPML1-lacking and PI(3,5)P₂-deficient cells exhibit defects in LEL-to-Golgi retrograde trafficking and autophagosome-lysosome fusion, suggesting that the TRPML1-PI(3,5)P₂ system represents a common signaling pathway essential for late endocytic trafficking.

Due to the function of lysosome in lysosomal trafficking, lysosomes are required for quality-control regulation of mitochondria, the “power house” of the cell and the major source of endogenous ROS (reactive oxygen species). Damaged mitochondria causes oxidative stress, which is a common feature of most LSDs, neurodegenerative diseases, and ageing (Xu, et al. 2015 Annu Rev Physiol 77, 57-80). Recent studies suggest that mitochondria are localized in close physical proximity to lysosomes (Elbaz-Alon, et al. 2014 Dev Cell 30, 95-102; Li, et al. 2015 Cell Mol Neurobiol 35, 615-621). Hence the lysosomal membrane is potentially an accessible and direct target of ROS signaling. Given that ROS reportedly regulate ion channels (Bogeski, et al. 2014 Antioxid Redox Signal 21, 859-862), it is possible that lysosomal conductance, particularly through lysosomal Ca²⁺ channels such as TRPML1, may mediate ROS-regulation of lysosomal function. Indeed, electrophysiological studies revealed that whole-endolysosome TRPML1 currents were directly activated by ROS.

A regulatory imbalance can result in elevated ROS levels and oxidative stress, which are believed to underlie a variety of metabolic and neurodegenerative diseases, as well as ageing (Barnham et al. 2004 Nat Rev Drug Discov 3, 205-214; Scherz-Shouval, et al. 2011 Trends Biochem Sci 36, 30-38). Given the role of TRPML1 in mediating ROS-induced autophagy, a TRPML1 agonist might be able to clear the excessive ROS, thereby ameliorating the ROS related diseases and ageing, especially photo ageing in the skin.

Transcription factor (TF)EB regulates autophagy and lysosome biogenesis. Overexpression of TFEB has been reportedly induce cellular clearance in a number of lysosome storage diseases, including Pompe Disease, Cystinosis, multiple sulfatase deficiency, as well as common neurodegenerative diseases, including Parkinson's disease and Huntinton's disease (Settembre, et al., 2013 Nat Rev Mol Cell Biol 14(5), 283-96). Therefore, activation of TRPML1 by TRPML1 agonists may also lead to cellular clearance in all the aforementioned diseases, providing therapeutic targets for these devastating diseases.

Previously, a potent synthetic agonist for TRPML1 has been reported (Shen, et al. 2012Nat Commun 3, 731). This SF-51-related compound (Mucolipin Synthetic Agonist 1 or ML-SA1) that could induce significant [Ca2+]_{cyt} increases in HEK293 cells stably or transiently expressing ML1-4A. In electrophysiological assays, ML-SA1 robustly activated whole-cell I_{ML1-4A} and whole-endolysosome I_{ML1}. ML-SA1 also activated whole-cell I_{TRPML2} and I_{TRPML3}, but not six other related channels. ML-SA1 (10 μM) activation of whole-endolysosome I_{ML1} was comparable to the effect of the endogenous TRPML agonist PI(3,5)P2 (1 μM), and these agonists were synergistic with each other. ML-SA1 activated an endogenous whole-endolysosome TRPML-like current (I_{ML-L}) in all mammalian cell types that were investigated, including Chinese Hamster Ovary (CHO), Cos-1, HEK293, skeletal muscle, pancreatic β and macrophage cells. ML-SA1 activated whole-endolysosome I_{ML-L} in wild-type (WT; ML1^{+/+}), but not ML4 (ML1^{-/-}) human fibroblasts, suggesting that although ML-SA1 targets all three TRPMLs, the expression levels of TRPML2 and TRPML3 are very low, and TRPML1 is the predominant lysosomal TRPML channel in this cell type. These results suggest that ML-SA1 is a reasonably specific and potent agonist that can be a useful for modulating the functions of TRPMLs.

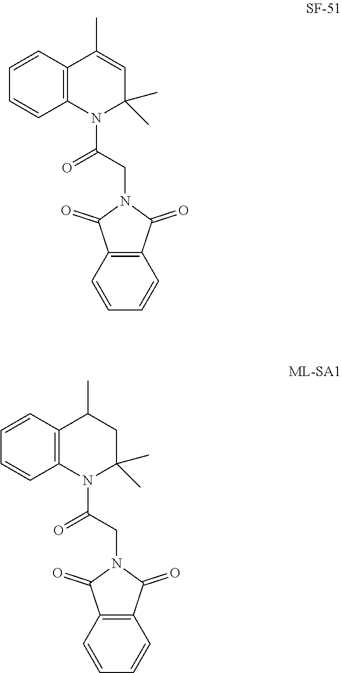 [SMILES: CC1=CC(C)(C)N(C(=O)CN2C(=O)c3ccccc3C2=O)c2ccccc21 ; CC1CC(C)(C)N(C(=O)CN2C(=O)c3ccccc3C2=O)c2ccccc21]

High concentrations of ML-SA1 (˜10 μM) are needed to effectively activate TRPMLs. Since that concentration is usually difficult to achieve in vivo, ML-SA1 cannot be used to treat the above TRPML related diseases.

Recently a number of more potent TRPML1 agonists have been developed (Qiu et al. WO 2022/076383 A1). However, most of these potent agonists are highly hydrophobic molecules that are metabolically labile and have very more solubility, which in turn leads to very poor oral bioavailability and systemic exposure.

There is an urgent need for more potent TRPML activators, in particular, compounds that are useful in treating disorders related to TRPML activities such as lysosome storage diseases, muscular dystrophy, age-related common neurodegenerative diseases, ROS or oxidative stress related diseases, metabolic diseases, metastatic cancer, and ageing.

### SUMMARY OF THE INVENTION

The invention is based in part on novel sulfonyl cyclic derivatives, pharmaceutical compositions thereof and methods of their preparation and use in treating or reducing various diseases or disorders. In particular, compounds, compositions and methods of the invention are useful in treating diseases or disorders mediated by or associated with TRPMLs.

In one aspect, the invention generally relates to a compound having the structural formula I:

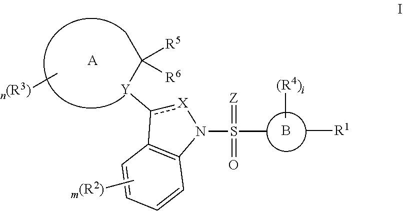

or a pharmaceutically acceptable form or an isotope derivative thereof, wherein
Ring A is a 4-, 5- or 6-membered substituted heterocycle;
Ring B a substituted or unsubstituted, 6-membered aryl or heteroaryl ring, 5-membered heteroaryl ring, or a substituted or unsubstituted bi- or multi-cyclic carbocyclic or heterocyclic ring;
X is CH or N;
Y is CH or N;
Z is O or NH;
R¹ is selected from the group consisting of C₁₋₅ alkyl, C₃₋₇ cycloalkyl, heterocyclic, halo, OR, SR, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, wherein the alkyl, cycloalkyl, heterocyclic, R and R′ is independently substituted with 0-6 F's;
each R² is independently selected from the group consisting of C₁₋₆ alkyl, halo, oxo, OH, CN, OR, NRR′, N(R)C(═O) RR′, N(R)C(═O)(O)RR′, OC(═O)NRR′, C(═O)R, C(═O)NRR′, N(R)S(O)₂RR′, S(O)₂R, or S(O)₂NRR′;
each R³ is independently selected from the group consisting of C₁₋₅ alkyl, C₃₋₇ cycloalkyl, heterocycle, heteroaryl, O, halo, CF₃, CH₂CF₃, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, which where applicable is optionally substituted with 1-3 groups selected from halo, CF₃, CH₂CF₃, CN, OH, C₁₋₅ alkyl, and C₃₋₇ cycloalkyl; two R³'s attached to the same carbon atom together form a C═O; or two R³'s, together with the carbon and/or nitrogen atom(s) to which they are attached, form a 3- to 6-membered substituted or unsubstituted carbocycle or heterocycle;
each R⁴ is independently selected from the group consisting of H, halo, CN, CF₃, C₁₋₅ alkyl, C₃₋₇ cycloalkyl and heterocyclic;
R⁵ and R⁶ are selected as follows:
   each of R⁵ and R⁶ is independently selected from C₁-C₃ alkyl, C₁-C₃ alkoxy, C₂-C₃ alkenyl, C₂-C₃ alkynyl, OH, CN, where applicable optionally substituted with 1-5 groups selected from C₁-C₃ alkyl, C₁-C₃ alkoxy, C₂-C₃ alkenyl, C₂-C₃ alkynyl, halo, OH and CN;
   one of R⁵ and R⁶ is CH₃, CH₂CH₃, OCH₃ or OCH₂CH₃ and the other is H;
   one of R⁵ and R⁶ is CF₃, CFHCH₃, CH₂CFH₂, CF₂CH₃, CFHCF₂H, CH₂CF₂H, CH₂CF₃, CFHCFH₂ or CF₂CF₃ and the other is H;
   one of R⁵ and R⁶ is CH═CH₂, CF═CH₂, CH═CFH, CH═CF₂, CF═CFH or CF═CF₂ and the other is H;
   one of R⁵ and R⁶ is C═CH, C═CF or CN and the other is H;
   one of R⁵ and R⁶ is a substituted or unsubstituted cyclopropyl, cyclobutyl or cyclopentyl ring and the other is H; or
   R⁵ and R⁶, together with the carbon atom to which they are attached to, are linked to form a substituted or unsubstituted 3- to 6-membered carbocyclic or heterocyclic ring;
each of R and R′ is independently H, or C₁₋₆ alkyl or cycloalkyl, optionally, R and R′, together with the nitrogen or carbon atom to which they are attached, form a 3- to 6-membered ring, each optionally substituted with 0-3 substituents independently selected from the group consisting of C₁₋₃ alkyl, halo, OH, OC₁₋₃ alkyl, and CN;
m is 0, 1, 2, 3 or 4;
n is 0, 1, 2, 3, 4 or 5; and
i is 0, 1 or 2.

In another aspect, the invention generally relates to a pharmaceutical composition comprising a compound disclosed herein.

In yet another aspect, the invention generally relates to a pharmaceutical composition comprising a compound of structural formula I.

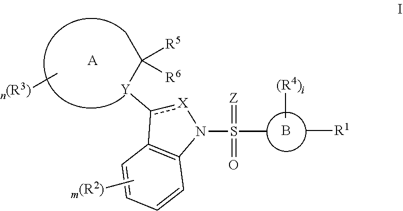

or a pharmaceutically acceptable form or an isotope derivative thereof, wherein
Ring A is a 4-, 5- or 6-membered substituted heterocycle;
Ring B a substituted or unsubstituted, 6-membered aryl or heteroaryl ring, 5-membered heteroaryl ring, or a substituted or unsubstituted bi- or multi-cyclic carbocyclic or heterocyclic ring;
X is CH or N;
Y is CH or N;
Z is O or NH;
R¹ is selected from the group consisting of C₁₋₅ alkyl, C₃₋₇ cycloalkyl, heterocyclic, halo, OR, SR, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, wherein the alkyl, cycloalkyl, heterocyclic, R and R′ is independently substituted with 0-6 F's;
each R² is independently selected from the group consisting of C₁₋₆ alkyl, halo, oxo, OH, CN, OR, NRR′, N(R)C(═O) RR′, N(R)C(═O)(O)RR′, OC(═O)NRR′, C(═O)R, C(═O)NRR′, N(R)S(O)₂RR′, S(O)₂R, or S(O)₂NRR′;
each R³ is independently selected from the group consisting of C₁₋₅ alkyl, C₃₋₇ cycloalkyl, heterocycle, heteroaryl, O, halo, CF₃, CH₂CF₃, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, which where applicable is optionally substituted with 1-3 groups selected from halo, CF₃, CH₂CF₃, CN, OH, C₁₋₅ alkyl, and C₃₋₇ cycloalkyl; two R³'s attached to the same carbon atom together form a C═O; or two R³'s, together with the carbon and/or nitrogen atom(s) to which they are attached, form a 3- to 6-membered substituted or unsubstituted carbocycle or heterocycle;
each R⁴ is independently selected from the group consisting of H, halo, CN, CF₃, C₁₋₅ alkyl, C₃₋₇ cycloalkyl and heterocyclic;
R⁵ and R⁶ are selected as follows:
   each of R⁵ and R⁶ is independently selected from C₁-C₃ alkyl, C₁-C₃ alkoxy, C₂-C₃ alkenyl, C₂-C₃ alkynyl, OH, CN, where applicable optionally substituted with 1-5 groups selected from C₁-C₃ alkyl, C₁-C₃ alkoxy, C₂-C₃ alkenyl, C₂-C₃ alkynyl, halo, OH and CN;
   one of R⁵ and R⁶ is CH₃, CH₂CH₃, OCH₃ or OCH₂CH₃ and the other is H;
   one of R⁵ and R⁶ is CF₃, CFHCH₃, CH₂CFH₂, CF₂CH₃, CFHCF₂H, CH₂CF₂H, CH₂CF₃, CFHCFH₂ or CF₂CF₃ and the other is H;
   one of R⁵ and R⁶ is CH═CH₂, CF═CH₂, CH═CFH, CH═CF₂, CF═CFH or CF═CF₂ and the other is H;
   one of R⁵ and R⁶ is C═CH, C═CF or CN and the other is H;
   one of R⁵ and R⁶ is a substituted or unsubstituted cyclopropyl, cyclobutyl or cyclopentyl ring and the other is H; or
   R⁵ and R⁶, together with the carbon atom to which they are attached to, are linked to form a substituted or unsubstituted 3- to 6-membered carbocyclic or heterocyclic ring;
each of R and R′ is independently H, or C₁₋₆ alkyl or cycloalkyl, optionally, R and R′, together with the nitrogen or carbon atom to which they are attached, form a 3- to 6-membered ring, each optionally substituted with 0-3 substituents independently selected from the group consisting of C₁₋₃ alkyl, halo, OH, OC₁₋₃ alkyl, and CN;
m is 0, 1, 2, 3 or 4;
n is 0, 1, 2, 3, 4 or 5; and
i is 0, 1 or 2,
   effective to treat, or reduce one or more diseases or disorders, in a mammal, including a human, and a pharmaceutically acceptable excipient, carrier, or diluent.

In yet another aspect, the invention generally relates to a unit dosage form comprising a pharmaceutical composition disclosed herein.

In yet another aspect, the invention generally relates to a method for treating or reducing a disease or disorder, comprising administering to a subject in need thereof a pharmaceutical composition comprising a compound disclosed herein.

In yet another aspect, the invention generally relates to a method for treating or reducing the effect of aging comprising administering to a subject in need thereof a pharmaceutical composition comprising a compound disclosed herein.

In yet another aspect, the invention generally relates to a method for treating or reducing oxidative stress or ROS related diseases or disorder, comprising administering to a subject in need thereof an effective amount of a TRPML1 agonist or a composition comprising of a TRPML1 agonist.

In yet another aspect, the invention generally relates to a method for treating or reducing oxidative stress or ROS related diseases or disorder, comprising administering to a subject in need thereof a pharmaceutical composition comprising a compound disclosed herein.

In yet another aspect, the invention generally relates to use of a compound disclosed herein, and a pharmaceutically acceptable excipient, carrier, or diluent, in preparation of a medicament for treating a disease or disorder.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

The following terms, unless indicated otherwise according to the context wherein the terms are found, are intended to have the following meanings.

Ranges provided herein are understood to be shorthand for all of the values within the range. For example, a range of 1 to 16 is understood to include any number, combination of numbers, or sub-range from the group consisting 1, 2, 3, 4, 5, 6, 7, 8, 9, 10, 11, 12, 13, 14, 15 or 16.

Any compositions or methods disclosed herein can be combined with one or more of any of the other compositions and methods provided herein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Definitions of specific functional groups and chemical terms are described in more detail below. When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl. Where substituent groups are specified by their conventional chemical formulae, written from left to right, they equally encompass the chemically identical substituents that would result from writing the structure from right to left, e.g., —C(═O)—O— is equivalent to —O—C(═O)—.

Structures of compounds of the invention are limited by principles of chemical bonding known to those skilled in the art. Accordingly, where a group may be substituted by one or more of a number of substituents, such substitutions are selected so as to comply with principles of chemical bonding and to give compounds that are not inherently unstable and/or would be known to one of ordinary skill in the art as likely to be unstable under ambient conditions (e.g., aqueous, neutral, and several known physiological conditions).

As used in this specification and the appended claims, the singular forms “a,” “an,” and “the” include plural reference, unless the context clearly dictates otherwise.

As used herein, “at least” a specific value is understood to be that value and all values greater than that value.

As used herein, the terms “comprises,” “comprising”, or “having” when used to define compositions and methods, are intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

As used herein, the terms “disease” and “disorder” are used interchangeably and refer to any condition that damages or interferes with the normal function of a cell, tissue, or organ. As used herein, the term “hydrate” means a compound which further includes a stoichiometric or non-stoichiometric amount of water bound by non-covalent intermolecular forces.

As used herein, the term “pharmaceutically acceptable” refers to being suitable for use in contact with the tissues of humans and other mammals without undue toxicity, irritation, allergic response and the like, and are commensurate with a reasonable benefit/risk ratio. A “pharmaceutically acceptable form” of a disclosed compound includes, but is not limited to, pharmaceutically acceptable salts, esters, hydrates, solvates, polymorphs, isomers, prodrugs, and isotopically labeled derivatives thereof. In one embodiment, a “pharmaceutically acceptable form” includes, but is not limited to, pharmaceutically acceptable salts, esters, prodrugs and isotopically labeled derivatives thereof. In some embodiments, a “pharmaceutically acceptable form” includes, but is not limited to, pharmaceutically acceptable isomers and stereoisomers, prodrugs and isotopically labeled derivatives thereof.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability. As such, those of skill in the art will appreciate that certain of the presently disclosed compounds having free amino, arnido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds having a carbonate, carbamate, amide or alkyl ester moiety covalently bonded to any of the above substituents disclosed herein.

Particularly favored prodrugs and prodrug salts are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or central nervous system) relative to the parent species. Examples of prodrugs include derivatives where a group that enhances aqueous solubility or active transport through the gut membrane is appended to the structure of formulae described herein. (See, e.g., Alexander, et al. 1988 J Med Chem 31, 318-322; Bundgaard, et al. 1985 Elsevier: Amsterdam 1-92; Bundgaard, et al. 1987 J Med Chem 30, 451-454; Bundgaard, H. A Textbook of Drug Design and Development; Harwood Academic Publ.: Switzerland, 1991, 113-191; Digenis, et al. Handbook of Experimental Pharmacology 1975, 28, 86-112; Friis, et al. Textbook of Drug Design and Development; 2 ed.; Overseas Publ.: Amsterdam, 1996, 351-385; Pitman 1981 Medicinal Research Reviews 1, 189-214.)

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “polymorph” means solid crystalline forms of a compound or complex thereof which may be characterized by physical means such as, for instance, X-ray powder diffraction patterns or infrared spectroscopy. Different polymorphs of the same compound can exhibit different physical, chemical and/or spectroscopic properties. Different physical properties include, but are not limited to stability (e.g., to heat, light or moisture), compressibility and density (important in formulation and product manufacturing), hygroscopicity, solubility, and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.
As used herein, the term “stable compounds” refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or disorder responsive to therapeutic agents).

As used herein, the term “stereoisomer” refers to both enantiomers and diastereomers. As used herein, the term “substantially free of other stereoisomers” means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing diastereomers are well known in the art and may be applied as practicable to final compounds or to starting material or intermediates. Other embodiments are those wherein the compound is an isolated compound. The term “at least X % enantiomerically enriched” as used herein means that at least X % of the compound is a single enantiomeric form, wherein X is a number between 0 and 100, inclusive.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. Treating or treatment thus refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving or stabilizing a patient's physical or mental well-being. The treatment or amelioration of symptoms can be based on objective or subjective parameters, for example, the results of a physical examination, neuropsychiatric exams, and/or a psychiatric evaluation. As compared with an equivalent untreated control, such reduction or degree of amelioration may be at least 5%, 10%, 20%, 40%, 50%, 60%, 80%, 90%, 95%, or 100% as measured by any standard technique.

As used herein, the terms “alk” or “alkyl” refer to straight or branched chain hydrocarbon groups having 1 to 12 carbon atoms, preferably 1 to 8 carbon atoms, containing no unsaturation. The expression “lower alkyl” refers to alkyl groups of 1 to 4 carbon atoms (inclusive). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₋₆ alkyl group. In some embodiments, “alkyl” can be a C₁₋₃ alkyl group.

As used herein, the term “alkenyl” refers to straight or branched chain hydrocarbon groups of 2 to 10, preferably 2 to 4, carbon atoms having at least one double bond. Where an alkenyl group is bonded to a nitrogen atom, it is preferred that such group not be bonded directly through a carbon bearing a double bond.

As used herein, the term “alkoxy” refers to an —O-alkyl radical.

As used herein, the term “alkynyl” refers to straight or branched chain hydrocarbon groups of 2 to 10, preferably 2 to 4, carbon atoms having at least one triple bond. Where an alkynyl group is bonded to a nitrogen atom, it is preferred that such group not be bonded directly through a carbon bearing a triple bond.

As used herein, the term “alkylene” refers to a divalent straight chain bridge of 1 to 5 carbon atoms connected by single bonds (e.g., —(CH₂)ₓ—, wherein x is 1 to 5), which may be substituted with 1 to 3 lower alkyl groups.

As used herein, the term “alkenylene” refers to a straight chain bridge of 2 to 5 carbon atoms having one or two double bonds that is connected by single bonds and may be substituted with 1 to 3 lower alkyl groups. Exemplary alkenylene groups are —CH═CH—CH═CH—, —CH₂—CH═CH—, —CH₂—CH═CH—CH₂—, —C(CH₃)₂CH═CH— and —CH(C₂H₅)—CH═CH—.

As used herein, the term “alkynylene” refers to a straight chain bridge of 2 to 5 carbon atoms that has a triple bond therein, is connected by single bonds, and may be substituted with 1 to 3 lower alkyl groups. Exemplary alkynylene groups are —C≡C—, —CH₂—C≡C—, —CH(CH₃)C≡C— and —C≡C≡CH(C₂H₅)CH₂—.

As used herein, the term “arylalkyl” refers to a moiety in which an alkyl hydrogen atom is replaced by an aryl group.

As used herein, the terms “cycloalkyl” and “cycloalkenyl” as employed herein includes saturated and partially unsaturated cyclic, respectively, hydrocarbon groups having 3 to 12 carbons, preferably 3 to 8 carbons, and more preferably 3 to 6 carbon.

As used herein, the terms “aromatic”, “ar” or “aryl” refer to a radical with 6 to 14 ring atoms (e.g., C₆₋₁₄ aromatic or C₆₋₁₄ aryl) that has at least one ring having a conjugated pi electron system which is carbocyclic (e.g., phenyl, fluorenyl, naphthyl, and anthracene). An aryl group may be, for example, 6 membered monocyclic, 10 membered bicyclic or 14 membered tricyclic ring systems, each with 6 to 14 carbon atoms.

As used herein, the term “halo” or “halogen” refers to any radical of fluorine, chlorine, bromine or iodine.

As used herein, the term “heteroaryl” or, alternatively, “heteroaromatic” refers to a refers to a radical of a 5-18 membered monocyclic or polycyclic (e.g., bicyclic, tricyclic, tetracyclic and the like) aromatic ring system (e.g., having 6, 10 or 14 π electrons shared in a cyclic array) having ring carbon atoms and 1-6 ring heteroatoms provided in the aromatic ring system, wherein each heteroatom is independently selected from nitrogen, oxygen, phosphorous and sulfur (“5-18 membered heteroaryl”). Heteroaryl polycyclic ring systems can include one or more heteroatoms in one or both rings. Whenever it appears herein, a numerical range such as “5 to 18” refers to each integer in the given range; e.g., “5 to 18 ring atoms” means that the heteroaryl group can consist of 5 ring atoms, 6 ring atoms, etc., up to and including 18 ring atoms. In some instances, a heteroaryl can have 5 to 14 ring atoms. In some embodiments, the heteroaryl has, for example, bivalent radicals derived from univalent heteroaryl radicals whose names end in “-yl” by removal of one hydrogen atom from the atom with the free valence are named by adding “-ene” to the name of the corresponding univalent radical, e.g., a pyridyl group with two points of attachment is a pyridylene. The term “heteroaryl”, for example, may refer to a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system, wherein 0, 1, 2, 3, or 4 atoms of each ring may be substituted by a substituent. Examples, without limitation, of heteroaryl groups are pyrrole, furan, thiophene, imidazole, oxazole, thiazole, pyrazole, pyridine, pyrimidine, quinoline, quinazoline, isoquinoline, purine and carbazole.

As used herein, the terms “heterocycle”, “heterocyclic” or “heterocyclo” refer to fully saturated or partially unsaturated cyclic groups, for example, 3 to 7 membered monocyclic, 7 to 12 membered bicyclic, or 10 to 15 membered tricyclic ring systems, which have at least one heteroatom in at least one ring, wherein 0, 1, 2 or 3 atoms of each ring may be substituted by a substituent. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system.

As used herein, the term “heterocyclyl” refers to fully saturated or partially unsaturated cyclic groups, for example, 3- to 7-membered monocyclic, 7- to 12-membered bicyclic, or 10- to 15-membered tricyclic ring systems, which have at least one heteroatom in at least one ring, wherein 0, 1, 2 or 3 atoms of each ring may be substituted by a substituent. Each ring of the heterocyclyl group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclyl group may be attached at any heteroatom or carbon atom of the ring or ring system.

As used herein, the term “oxo” refers to an oxygen atom, which forms a carbonyl when attached to carbon, an N-oxide when attached to nitrogen, and a sulfoxide or sulfone when attached to sulfur.

As used herein, the term “substituents” refers to a group “substituted” on any functional group delineated herein, e.g., alkyl, alkenyl, alkynyl, cycloalkyl, cycloalkenyl, aryl, heterocyclyl, or heteroaryl group at any atom of that group. Suitable substituents include, without limitation halogen, CN, NO₂, OR¹⁵, SR¹⁵, S(O)₂OR¹⁵, NR¹⁵R¹⁶, C₁-C₂ perfluoroalkyl, C₁-C₂ perfluoroalkoxy, 1,2-methylenedioxy, C(O)OR¹⁵, C(O)NR¹⁵R¹⁶, OC(O)NR¹⁵R¹⁶, NR¹⁵C(O)NR¹⁵R¹⁶, C(NR¹⁶)NR¹⁵R¹⁶, NR¹⁵C(NR¹⁶)NR¹⁵R¹⁶, S(O)₂NR¹⁵R¹⁶, R¹⁷, C(O)R¹⁷, NR¹⁵C(O)R¹⁷, S(O)R¹⁷, S(O)₂R¹⁷, R¹⁶, oxo, C(O)R¹⁶, C(O)(CH₂)ₘOH, (CH₂)ₙOR¹⁵, (CH₂)ₘC(O)NR¹⁵R¹⁶, NR¹⁵S(O)₂R¹⁷, where m is independently 0-6 inclusive. Each R¹⁵ is independently hydrogen, C₁-C₄ alkyl or C₃-C₆ cycloalkyl. Each R¹⁶ is independently hydrogen, alkenyl, alkynyl, C₃-C₆ cycloalkyl, aryl, heterocyclyl, heteroaryl, C₁-C₄ alkyl or C₁-C₄ alkyl substituted with C₃-C₆ cycloalkyl, aryl, heterocyclyl or heteroaryl. Each R¹⁷ is independently C₃-C₆ cycloalkyl, aryl, heterocyclyl, heteroaryl, C₁-C₄ alkyl or C₁-C₄ alkyl substituted with C₃-C₆ cycloalkyl, aryl, heterocyclyl or heteroaryl. Each C₃-C₆ cycloalkyl, aryl, heterocyclyl, heteroaryl and C₁-C₄ alkyl in each R¹⁵, R¹⁶ and R¹⁷ can optionally be substituted with halogen, CN, C₁-C₄ alkyl, OH, C₁-C₄ alkoxy, NH₂, C₁-C₄ alkylamino, C₁-C₄ dialkylamino, C₁-C₂ perfluoroalkyl, C₁-C₂ perfluoroalkoxy, or 1,2-methylenedioxy.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein. All such isomeric forms of such compounds are expressly included in the present invention. All crystal forms of the compounds described herein are expressly included in the present invention.

### DETAILED DESCRIPTION OF THE INVENTION

The invention provides novel sulfonyl cyclic derivatives, including salts, solvates, hydrates and polymorphs thereof, as TRPML modulators. The invention also provides pharmaceutical compositions comprising a compound of this invention and the use of such compositions in treating a range of diseases and conditions associated with TRPML or related to TRPML activities, such as lysosome storage diseases, muscular dystrophy, neurodegenerative diseases, ROS or oxidative stress related diseases, and damages caused in skin or photoaging.

In one aspect, the invention generally relates to a compound having the structure of formula I:

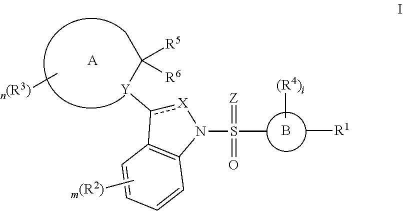

or a pharmaceutically acceptable form or an isotope derivative thereof, wherein
Ring A is a 4-, 5- or 6-membered substituted heterocycle;
Ring B a substituted or unsubstituted, 6-membered aryl or heteroaryl ring, 5-membered heteroaryl ring, or a substituted or unsubstituted bi- or multi-cyclic carbocyclic or heterocyclic ring;
X is CH or N;
Y is CH or N;
Z is O or NH;
R¹ is selected from the group consisting of C₁₋₅ alkyl, C₃₋₇ cycloalkyl, heterocyclic, halo, OR, SR, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, wherein the alkyl, cycloalkyl, heterocyclic, R and R′ is independently substituted with 0-6 (e.g., 0, 1, 2, 3, 4, 5 or 6) F's;
each R² is independently selected from the group consisting of C₁₋₆ alkyl, halo, oxo, OH, CN, OR, NRR′, N(R)C(═O) RR′, N(R)C(═O)(O)RR′, OC(═O)NRR′, C(═O)R, C(═O)NRR′, N(R)S(O)₂RR′, S(O)₂R, or S(O)₂NRR′;
each R³ is independently selected from the group consisting of C₁₋₅ alkyl, C₃₋₇ cycloalkyl, heterocycle, heteroaryl, O, halo, CF₃, CH₂CF₃, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, which where applicable is optionally substituted with 1, 2 or 3 groups selected from halo, CF₃, CH₂CF₃, CN, OH, C₁₋₅ alkyl, and C₃₋₇ cycloalkyl; two R³'s attached to the same carbon atom together form a C═O; or two R³'s, together with the carbon and/or nitrogen atom(s) to which they are attached, form a 3-, 4-, 5- or 6-membered substituted or unsubstituted carbocycle or heterocycle;
each R⁴ is independently selected from the group consisting of H, halo, CN, CF₃, C₁₋₅ alkyl, C₃₋₇ cycloalkyl and heterocyclic;
R⁵ and R⁶ are selected as follows:
   each of R⁵ and R⁶ is independently selected from C₁-C₃ alkyl, C₁-C₃ alkoxy, C₂-C₃ alkenyl, C₂-C₃ alkynyl, OH, CN, where applicable optionally substituted with 1-5 (e.g., 1, 2, 3, 4 or 5) groups selected from C₁-C₃ alkyl, C₁-C₃ alkoxy, C₂-C₃ alkenyl, C₂-C₃ alkynyl, halo, OH and CN;
   one of R⁵ and R⁶ is CH₃, CH₂CH₃, OCH₃ or OCH₂CH₃ and the other is H;
   one of R⁵ and R⁶ is CF₃, CFHCH₃, CH₂CFH₂, CF₂CH₃, CFHCF₂H, CH₂CF₂H, CH₂CF₃, CFHCFH₂ or CF₂CF₃ and the other is H;
   one of R⁵ and R⁶ is CH═CH₂, CF═CH₂, CH═CFH, CH═CF₂, CF═CFH or CF═CF₂ and the other is H;
   one of R⁵ and R⁶ is C═CH, C═CF or CN and the other is H;
   one of R⁵ and R⁶ is a substituted or unsubstituted cyclopropyl, cyclobutyl or cyclopentyl ring and the other is H; or
   R⁵ and R⁶, together with the carbon atom to which they are attached to, are linked to form a substituted or unsubstituted 3- to 6-membered carbocyclic or heterocyclic ring;
each of R and R′ is independently H, or C₁₋₆ alkyl or cycloalkyl, optionally, R and R′, together with the nitrogen or carbon atom to which they are attached, form a 3- to 6-membered ring, each optionally substituted with 0-3 (e.g., 0, 1, 2 or 3) substituents independently selected from the group consisting of C₁₋₃ alkyl, halo, OH, OC₁₋₃ alkyl, and CN;
m is 0, 1, 2, 3 or 4;
n is 0, 1, 2, 3, 4 or 5; and
i is 0, 1 or 2.

In certain embodiments of (I), Ring B is a substituted or unsubstituted 6-membered aryl.

In certain embodiments, Ring B is a substituted or unsubstituted phenyl.

In certain embodiments, i is 0 and Ring B is unsubstituted phenyl:

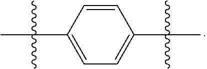 [SMILES: *c1ccc(*)cc1]

In certain embodiments, Ring B is a substituted or unsubstituted 6-membered heteroaryl.

In certain embodiments, Ring B is a substituted or unsubstituted pyridinyl. In certain embodiments, i is 0 and Ring B is an unsubstituted pyridinyl:

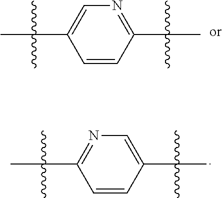

In certain embodiments, Ring B is a substituted or unsubstituted pyrimidinyl, pyrazinyl or pyridazinyl.

In certain embodiments, Ring B is a substituted or unsubstituted 5-membered heteroaryl.

In certain embodiments, Ring B is a substituted or unsubstituted thiophene. In certain embodiments, i is 0 and Ring B is unsubstituted thiophene:

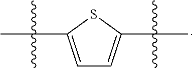 [SMILES: *c1ccc(*)s1]

In certain embodiments, Ring B is a substituted or unsubstituted pyrrolyl. In certain embodiments, i is 0 and Ring B is unsubstituted pyrrolyl:

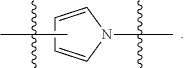

In certain embodiments, Ring B is a substituted or unsubstituted thiazolyl, pyrazolyl, imidazolyl or triazolyl.

In certain embodiments, Ring B is a substituted or unsubstituted bi- or multi-cyclic carbocyclic.

In certain embodiments, Ring B is bicyclo[1.1.1]pentane:

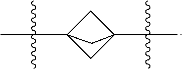

In certain embodiments, Ring B is a substituted or unsubstituted bi- or multi-cyclic heterocyclic.

In certain embodiments, Ring B is 2-oxabicyclo[2.2.2]octane.

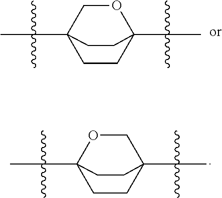

In certain embodiments, R¹ is S(O)₂CHRR′.

In certain embodiments, R¹ is S(O)₂C(CH₃)RR′.

In certain embodiments, R¹ is S(O)₂NRR′.

In certain embodiments of R¹, R and R′ is independently H, C₁₋₆ alkyl or cycloalkyl.

In certain embodiments of R¹, R and R′, together with the nitrogen or carbon atom to which they are attached, form a substituted or unsubstituted 3- to 6-membered carbocycle or heterocycle.

In certain embodiments, R¹ is halo (e.g., F, Cl).

In certain embodiments, R¹ is a C₁₋₅ alkyl, C₃₋₇cycloalkyl or heterocyclic, wherein the alkyl, cycloalkyl or heterocyclic is substituted with 0-6 F's.

In certain embodiments, R¹ is a C₁₋₅ alkyl, substituted with 0-6 (e.g., 0, 1, 2, 3, 4, 5 or 6) F's.

In certain embodiments, R¹ is a C₁₋₃ alkyl, substituted with 0-6 (e.g., 0, 1, 2, 3, 4, 5 or 6) F's.

In certain embodiments, R¹ is OR or SR, wherein R is substituted with 0-6 (e.g., 0, 1, 2, 3, 4, 5 or 6) F's.

Non-limiting examples of include:

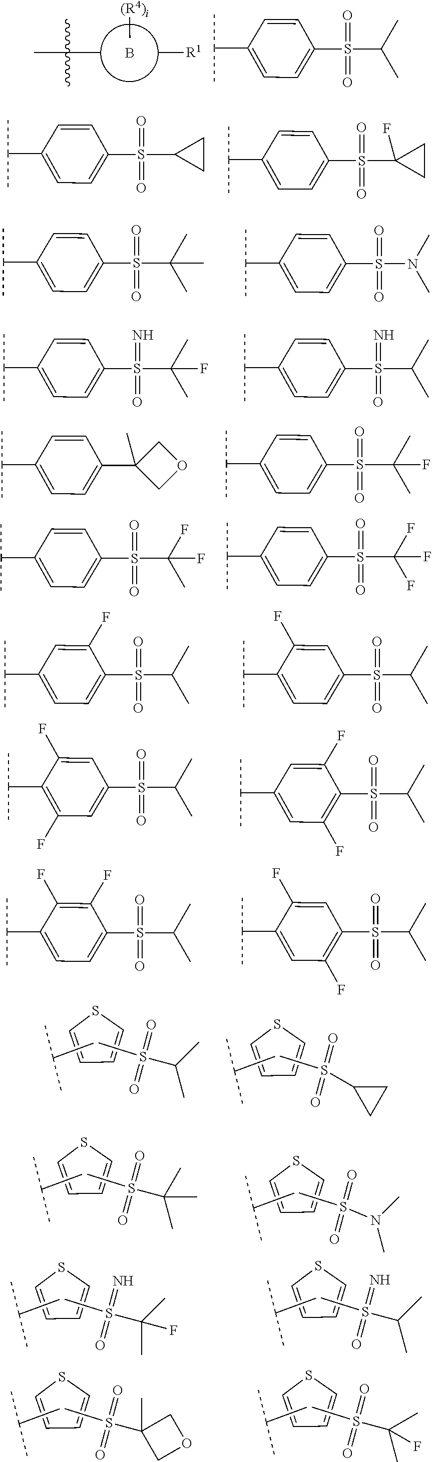

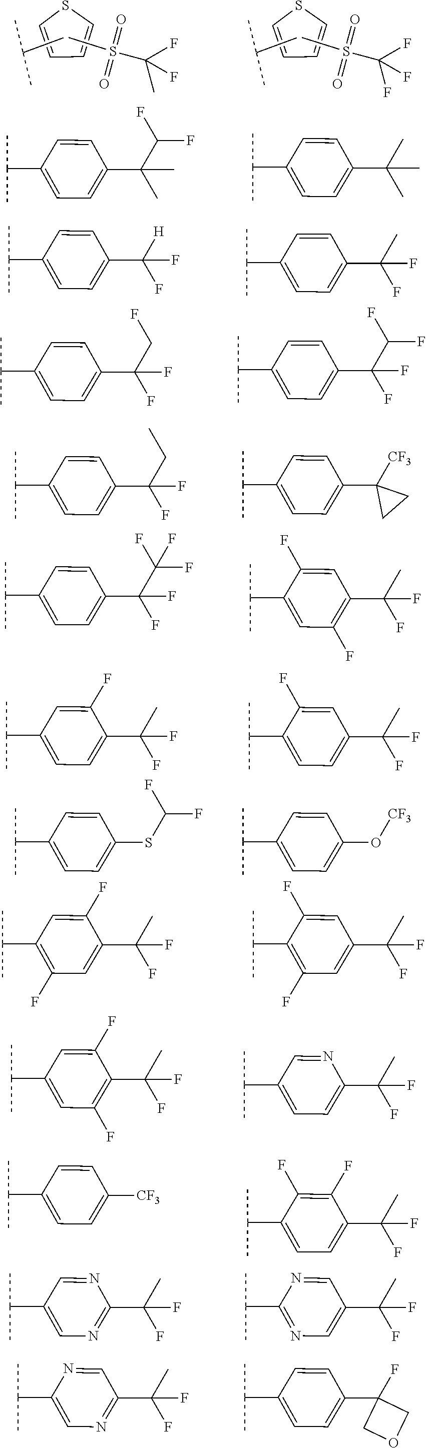

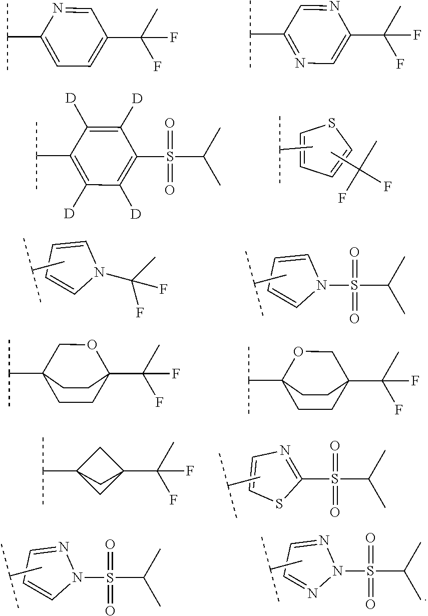

In certain embodiments of (I), X is N.

In certain embodiments of (I), X is CH.

In certain embodiments of (I), Y is N.

In certain embodiments of (I), Y is CH.

In certain embodiments of (I), Z is O.

In certain embodiments of (I), Z is NH.

In certain embodiments of (I), X is N, Y is N, and Z is O:

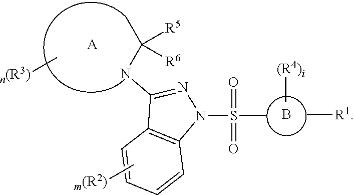

In certain embodiments of (I), Ring A is a 4-membered substituted heterocycle.

In certain embodiments of (I), Ring A is a 5-membered substituted heterocycle.

In certain embodiments of (I), Ring A is a 6-membered substituted heterocycle.

In certain embodiments, Ring A is:

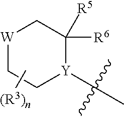

wherein
W is NR⁷, CR⁷R^{7′}, O or C(O); and
R⁷ and R^{7′} is independently selected from the group consisting of H, halo, CF₃, CH₂CF₃, CN, OH, C₁₋₅ alkyl, C₁₋₃ alkoxy, C₃₋₇ cycloalkyl, and 5-, 6- or 7-membered aryl or heteroaryl, optionally substituted with 1, 2 or 3 groups selected from halo, CF₃, CH₂CF₃, CN, OH, C₁₋₅ alkyl, and C₁₋₃ alkoxy; or R⁷ and R^{7′}, together with the carbon and/or nitrogen atom(s) to which they are attached, form a 3-, 4-, 5- or 6-membered substituted or unsubstituted carbocycle or heterocycle.

In certain embodiments, Ring A has a structural formula selected from:

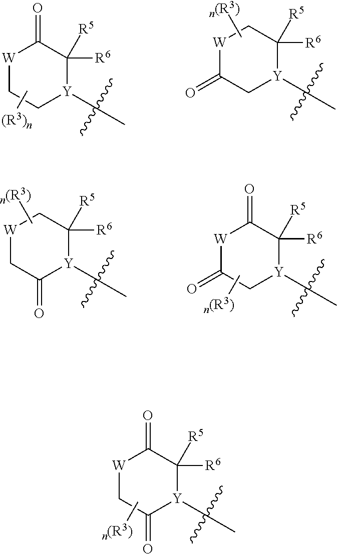

wherein W is NR⁷, CR⁷R^{7′} or O.

In certain embodiments, Ring A has a structural formula selected from:

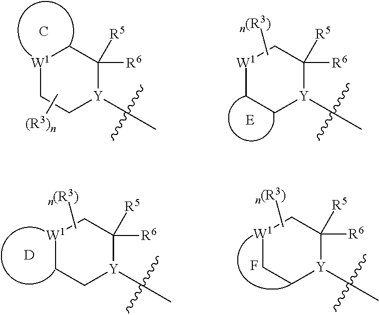

wherein
W¹ is NR⁷ or CR⁷R^{7′}; and
each of Ring C, Ring D, Ring E and Ring F is a 3-, 4-, 5- or 6-membered substituted or unsubstituted carbocycle or heterocycle.

In certain embodiments, Ring A is:

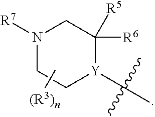

In certain embodiments, Ring A is:

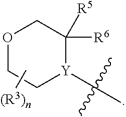

In certain embodiments, Ring A is:

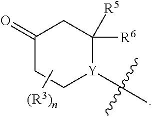

In certain embodiments, Ring A is:

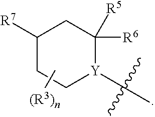

In certain embodiments, each of R⁵ and R⁶ is independently selected from C₁-C₃ alkyl, C₁-C₃ alkoxy, C₂-C₃ alkenyl, C₂-C₃ alkynyl, OH, CN, where applicable optionally substituted with 1-5 (e.g., 1, 2, 3, 4 or 5) groups selected from C₁-C₃ alkyl, C₁-C₃ alkoxy, C₂-C₃ alkenyl, C₂-C₃ alkynyl, halo, OH and CN.

In certain embodiments, each of R⁵ and R⁶ is independently selected from CH₃, CH₂CH₃, OCH₃, OCH₂CH₃, CF₃, CFHCH₃, CH₂CFH₂, CF₂CH₃, CFHCF₂H, CH₂CF₂H, CH₂CF₃, CFHCFH₂, CF₂CF₃, CH═CH₂, CF═CH₂, CH═CFH, CH═CF₂, CF═CFH, CF═CF₂, C≡CH, C≡CF, OH and CN.

In certain embodiments, each of R⁵ and R⁶ is CH₃ or CH₂CH₃. In certain embodiments, each of R⁵ and R⁶ is CH₃.

In certain embodiments, one of R⁵ and R⁶ is CH₃, CH₂CH₃, OCH₃ or OCH₂CH₃ and the other is H.

In certain embodiments, one of R⁵ and R⁶ is CF₃, CFHCH₃, CH₂CFH₂, CF₂CH₃, CFHCF₂H, CH₂CF₂H, CH₂CF₃, CFHCFH₂ or CF₂CF₃ and the other is H. In certain embodiments, one of R⁵ and R⁶ is CF₃ and the other is H.

In certain embodiments, one of R⁵ and R⁶ is CH═CH₂, CF═CH₂, CH═CFH, CH═CF₂, CF═CFH or CF═CF₂ and the other is H.

In certain embodiments, one of R⁵ and R⁶ is C═CH, C≡CF or CN and the other is H.

In certain embodiments, one of R⁵ and R⁶ is a substituted or unsubstituted cyclopropyl, cyclobutyl or cyclopentyl ring and the other is H.

In certain embodiments, R⁵ and R⁶, together with the carbon atom to which they are attached to, are linked to form a substituted or unsubstituted 3- to 6-membered carbocyclic or heterocyclic ring (e.g., cyclopropyl ring).

In certain embodiments, n is 0 and Ring A is selected from:

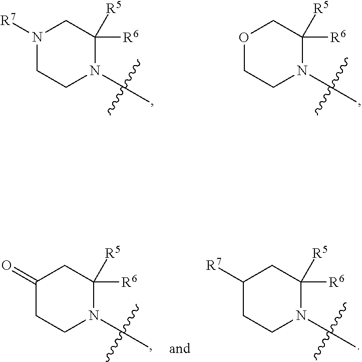

In certain embodiments, Ring A is:

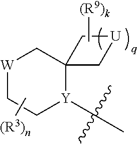

wherein
each U is independently selected from CH₂ and O; and
q is 0, 1, 2 or 3.

In certain embodiments, U is CH₂ and q is 0, 1 or 2.

In certain embodiments, U is O and q is 1.

In certain embodiments, Ring A is:

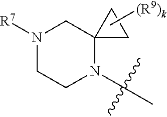

wherein
R⁹ is selected from halo, CN, CH₃, CH₂F, CHF₂ or OCH₃, optionally substituted with OR; and
k is 0, 1, 2, 3 or 4.

In certain embodiments, k is 0.

In certain embodiments, k is 1.

Non-limiting examples of Ring A include:

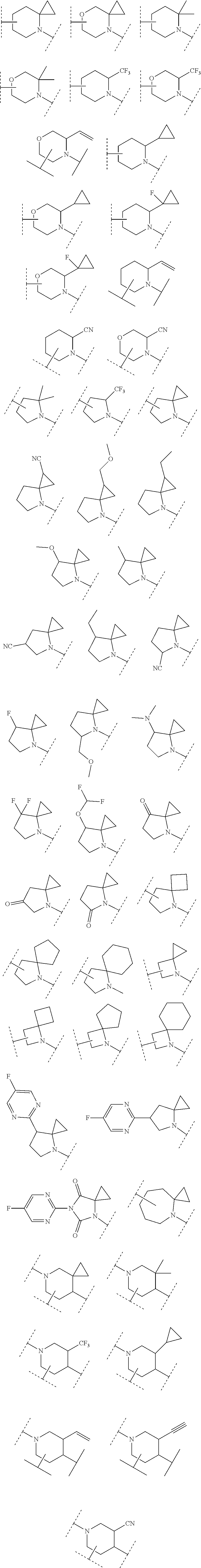

Additional non-limiting examples of Ring A include:

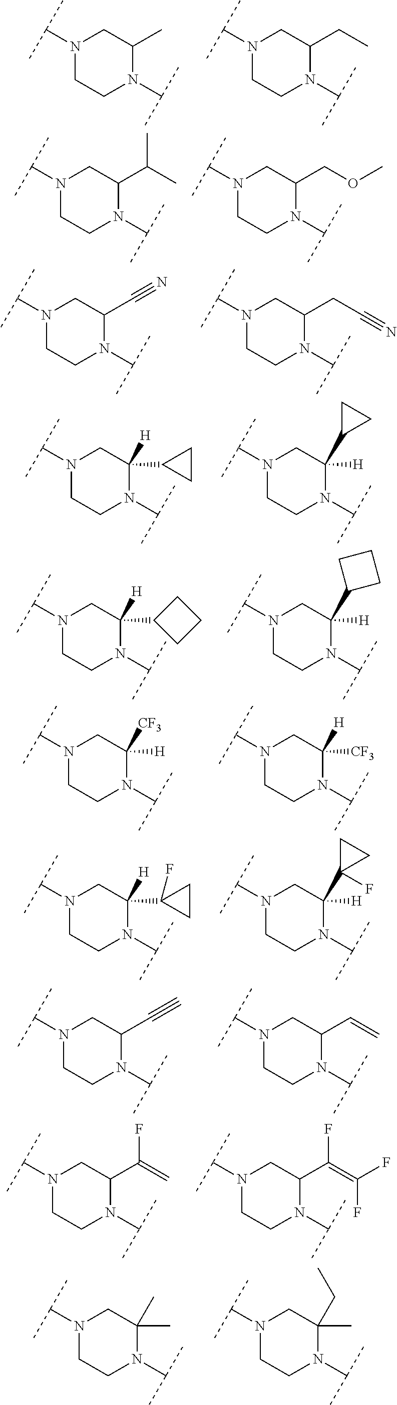

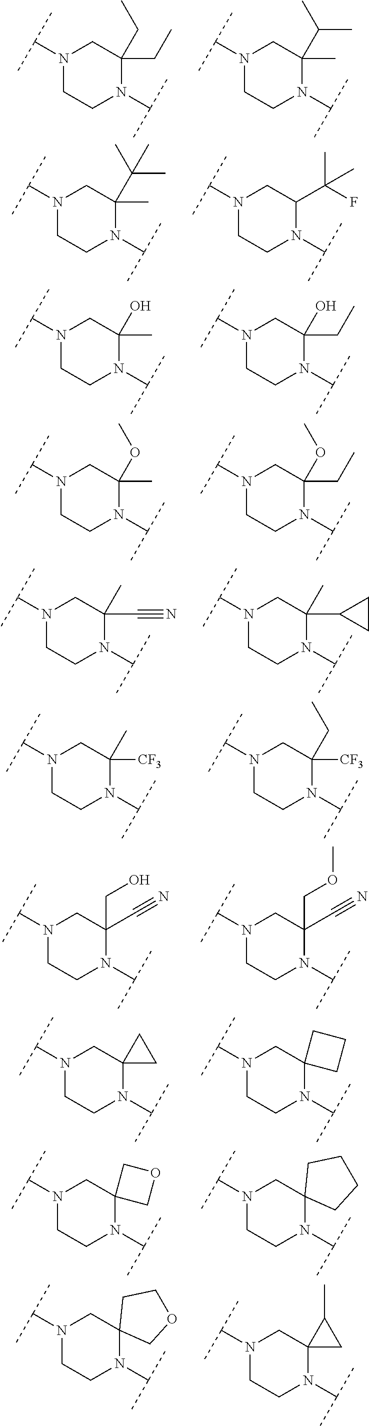

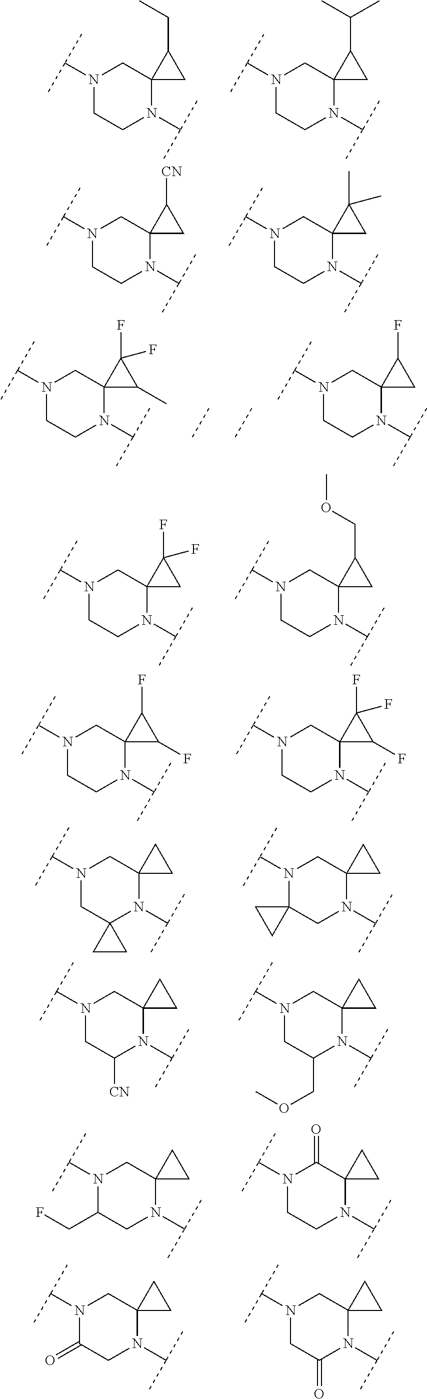

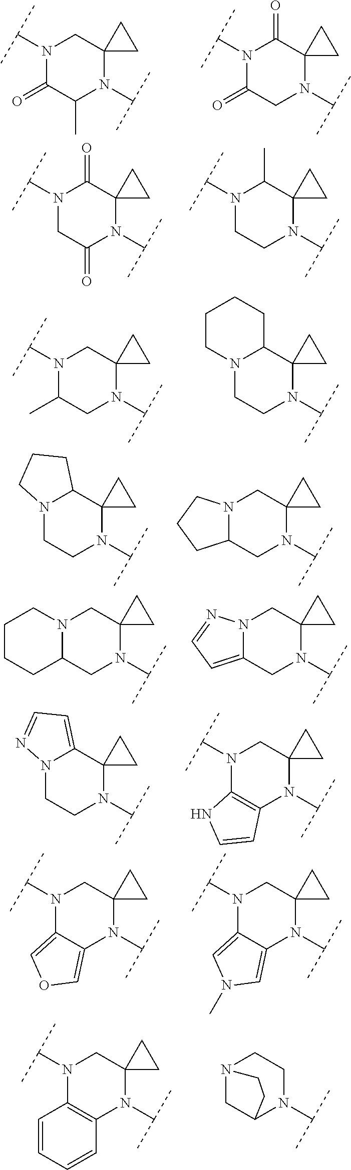

In certain embodiments, R⁷ is:

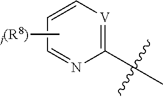

wherein
V is N or CH, optionally substituted with a halo or C₁-C₃ alkyl;
R⁸ is halo, CF₃, CH₂CF₃, CN, OH, C₁₋₅ alkyl, or C₃₋₇ cycloalkyl; and
j is 0, 1 or 2.

In certain embodiments, V is N:

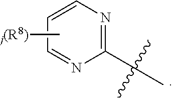

In certain embodiments, j is 1.

In certain embodiments, R⁸ has the following positioning:

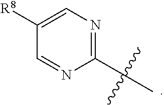

In certain embodiments, R⁸ is F or Cl.

In certain embodiments, V is CH, optionally substituted with a halo or C₁-C₃ alkyl.

In certain embodiments, V is C—F or C—Cl:

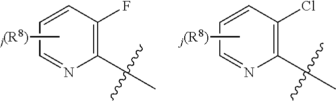

In certain embodiments, j is 1.

In certain embodiments, R⁸ has the following positioning:

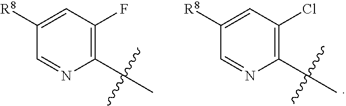

In certain embodiments, R⁸ is F or Cl.

In certain embodiments, a compound of the invention has the structure of:

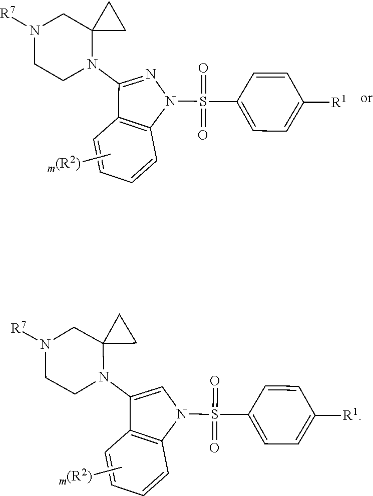

In certain embodiments, a compound of the invention has the structure of:

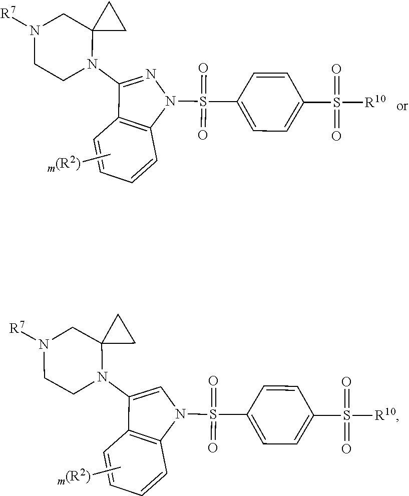

wherein R¹⁰ is NRR′, CHRR′ or CCH₃RR′.

In certain embodiments, a compound of the invention has the structure of:

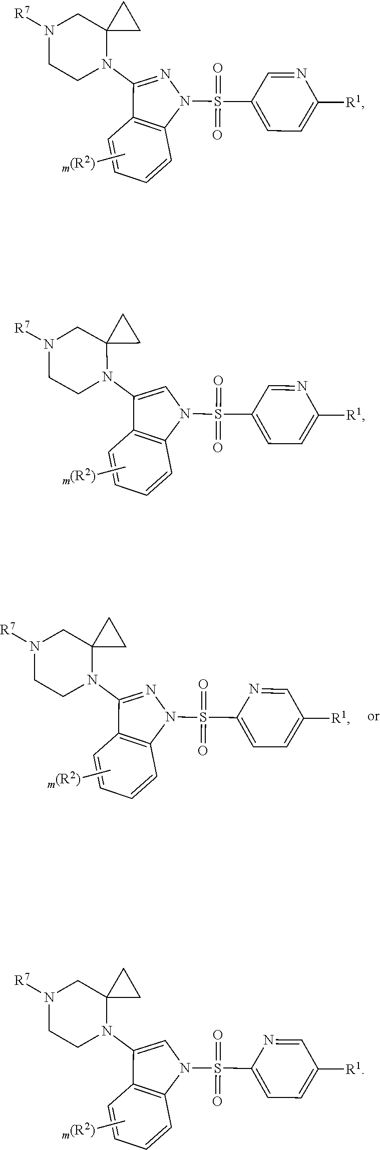

In certain embodiments, a compound of the invention has the structure of:

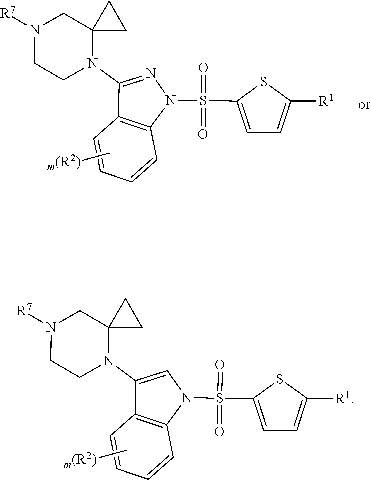

In certain embodiments, a compound of the invention has the structure of:

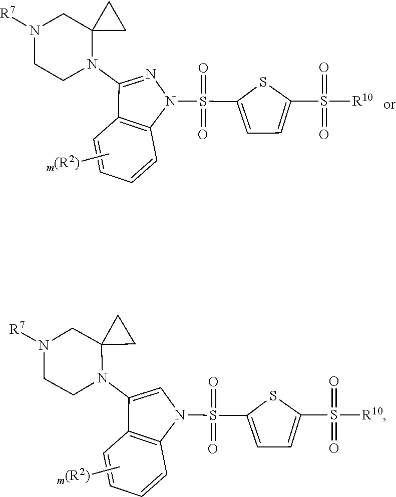

wherein R¹⁰ is NRR′, CHRR′ or CCH₃RR′.

In certain embodiments, a compound of the invention has the structure of:

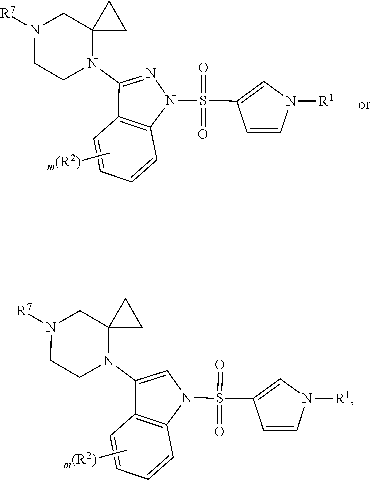

wherein R is C₁₋₅ alkyl, C₃₋₇ cycloalkyl, heterocyclic or S(O)₂R, wherein the alkyl, cycloalkyl, heterocyclic and R is independently substituted with 0-6 (e.g., 0, 1, 2, 3, 4, 5 or 6) F's.

In certain embodiments, a compound of the invention has the structure of:

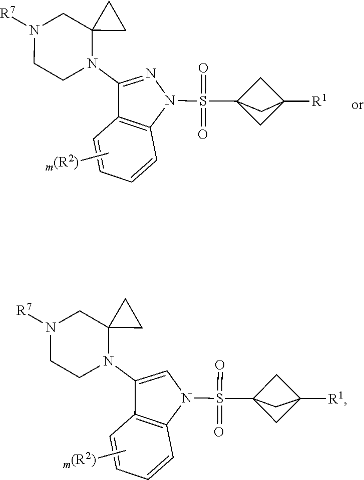

wherein R¹ is halo, C₁₋₅ alkyl or C₃₋₇ cycloalkyl, wherein the alkyl or cycloalkyl is independently substituted with 0-6 (e.g., 0, 1, 2, 3, 4, 5 or 6) F's.

In certain embodiments, R⁷ is:

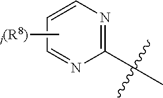

wherein
R⁸ is halo, CF₃, CH₂CF₃, or C₁₋₅ alkyl; and
j is 0, 1 or 2.

In certain embodiments, j is 1 and R⁷ is:

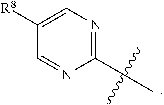

In certain embodiments, R⁸ is F.

In certain embodiments, R⁸ is Cl.

In certain embodiments, R¹⁰ is CHCH₃CH₃.

In certain embodiments, R¹⁰ is C(CH₃)₃.

In certain embodiments, R¹⁰ is NCH₃CH₃.

In certain embodiments, m is 0.

In certain embodiments, m is 1.

In certain embodiments, m is 2.

In certain embodiments, R² has a positioning selected from:

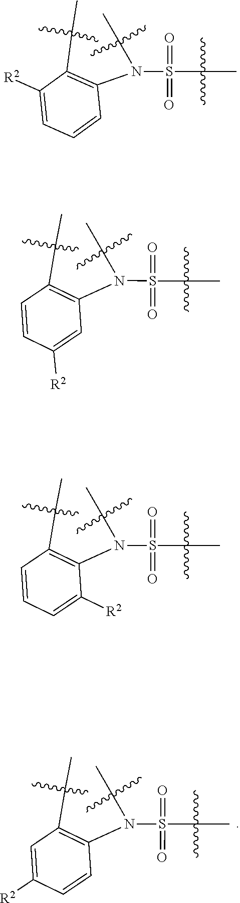

In certain embodiments, R² is independently a halo.

In certain embodiments, R²'s have positionings selected from:

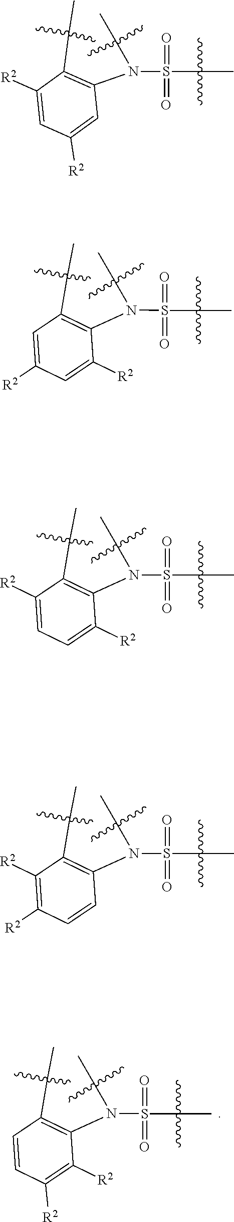

In certain embodiments, each R² is independently selected from F and Cl.

Exemplary compounds of the invention include but not limited to:

TABLE 1
Exemplary Compounds

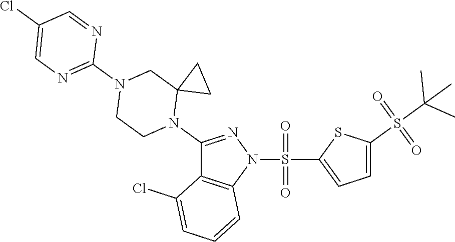 [SMILES: CC(C)(C)S(=O)(=O)c1ccc(S(=O)(=O)n2nc(N3CCN(c4ncc(Cl)cn4)CC34CC4)c3c(Cl)cccc32)s1]

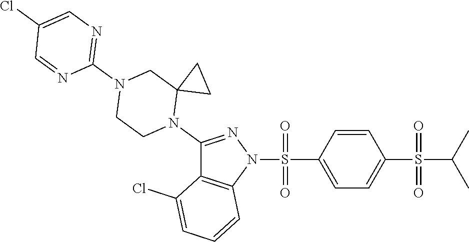 [SMILES: CC(C)S(=O)(=O)c1ccc(S(=O)(=O)n2nc(N3CCN(c4ncc(Cl)cn4)CC34CC4)c3c(Cl)cccc32)cc1]

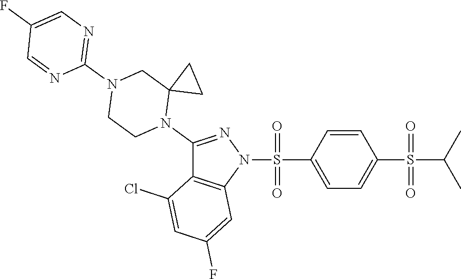 [SMILES: CC(C)S(=O)(=O)c1ccc(S(=O)(=O)n2nc(N3CCN(c4ncc(F)cn4)CC34CC4)c3c(Cl)cc(F)cc32)cc1]

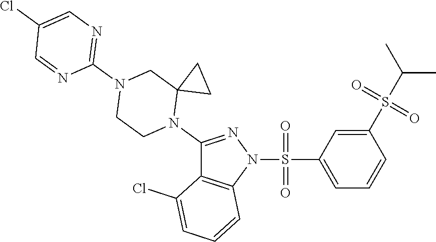 [SMILES: CC(C)S(=O)(=O)c1cccc(S(=O)(=O)n2nc(N3CCN(c4ncc(Cl)cn4)CC34CC4)c3c(Cl)cccc32)c1]

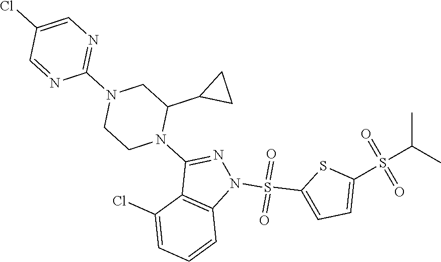 [SMILES: CC(C)S(=O)(=O)c1ccc(S(=O)(=O)n2nc(N3CCN(c4ncc(Cl)cn4)CC3C3CC3)c3c(Cl)cccc32)s1]

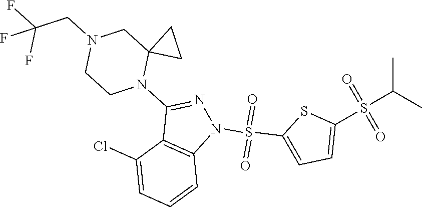 [SMILES: CC(C)S(=O)(=O)c1ccc(S(=O)(=O)n2nc(N3CCN(CC(F)(F)F)CC34CC4)c3c(Cl)cccc32)s1]

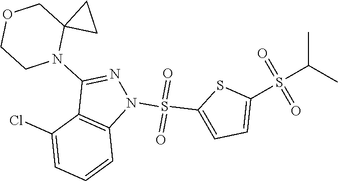 [SMILES: CC(C)S(=O)(=O)c1ccc(S(=O)(=O)n2nc(N3CCOCC34CC4)c3c(Cl)cccc32)s1]

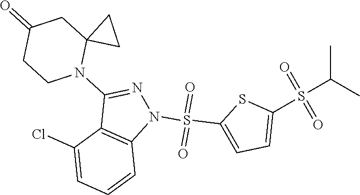 [SMILES: CC(C)S(=O)(=O)c1ccc(S(=O)(=O)n2nc(N3CCC(=O)CC34CC4)c3c(Cl)cccc32)s1]

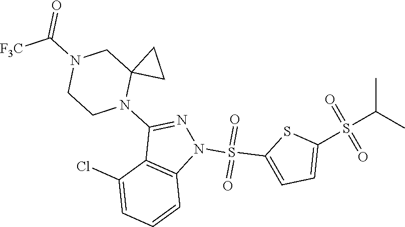 [SMILES: CC(C)S(=O)(=O)c1ccc(S(=O)(=O)n2nc(N3CCN(C(=O)C(F)(F)F)CC34CC4)c3c(Cl)cccc32)s1]

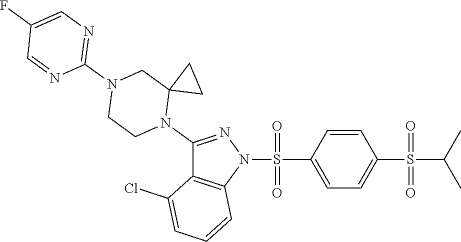 [SMILES: CC(C)S(=O)(=O)c1ccc(S(=O)(=O)n2nc(N3CCN(c4ncc(F)cn4)CC34CC4)c3c(Cl)cccc32)cc1]

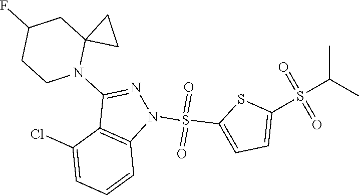 [SMILES: CC(C)S(=O)(=O)c1ccc(S(=O)(=O)n2nc(N3CCC(F)CC34CC4)c3c(Cl)cccc32)s1]

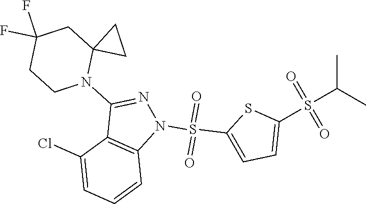 [SMILES: CC(C)S(=O)(=O)c1ccc(S(=O)(=O)n2nc(N3CCC(F)(F)CC34CC4)c3c(Cl)cccc32)s1]

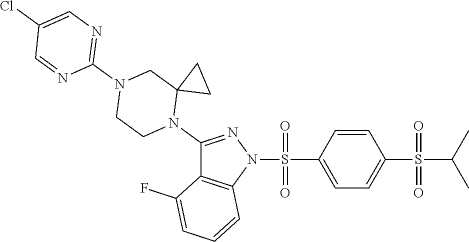 [SMILES: CC(C)S(=O)(=O)c1ccc(S(=O)(=O)n2nc(N3CCN(c4ncc(Cl)cn4)CC34CC4)c3c(F)cccc32)cc1]

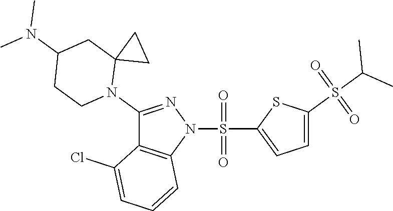 [SMILES: CC(C)S(=O)(=O)c1ccc(S(=O)(=O)n2nc(N3CCC(N(C)C)CC34CC4)c3c(Cl)cccc32)s1]

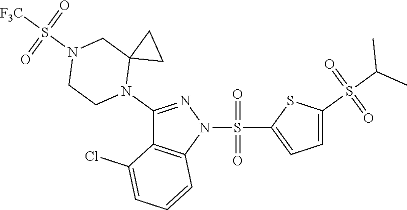 [SMILES: CC(C)S(=O)(=O)c1ccc(S(=O)(=O)n2nc(N3CCN(S(=O)(=O)C(F)(F)F)CC34CC4)c3c(Cl)cccc32)s1]

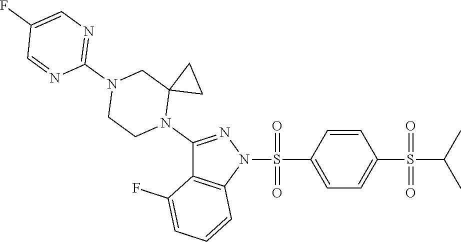 [SMILES: CC(C)S(=O)(=O)c1ccc(S(=O)(=O)n2nc(N3CCN(c4ncc(F)cn4)CC34CC4)c3c(F)cccc32)cc1]

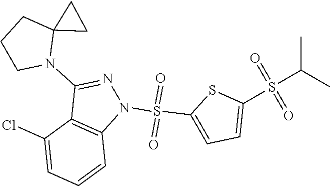 [SMILES: CC(C)S(=O)(=O)c1ccc(S(=O)(=O)n2nc(N3CCCC34CC4)c3c(Cl)cccc32)s1]

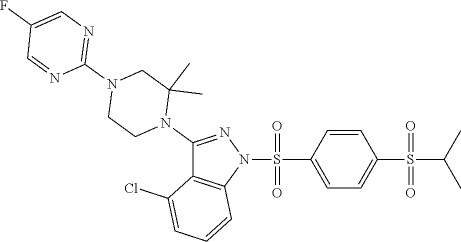 [SMILES: CC(C)S(=O)(=O)c1ccc(S(=O)(=O)n2nc(N3CCN(c4ncc(F)cn4)CC3(C)C)c3c(Cl)cccc32)cc1]

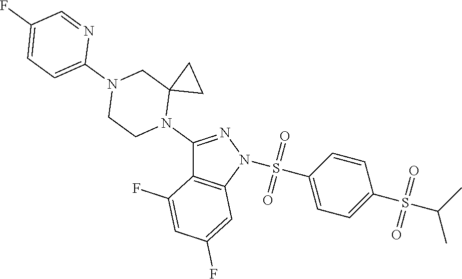 [SMILES: CC(C)S(=O)(=O)c1ccc(S(=O)(=O)n2nc(N3CCN(c4ccc(F)cn4)CC34CC4)c3c(F)cc(F)cc32)cc1]

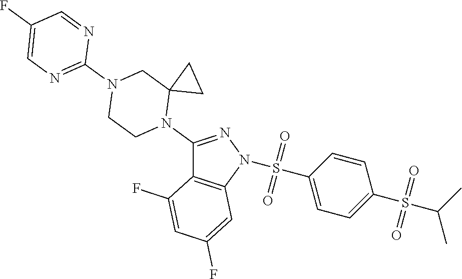 [SMILES: CC(C)S(=O)(=O)c1ccc(S(=O)(=O)n2nc(N3CCN(c4ncc(F)cn4)CC34CC4)c3c(F)cc(F)cc32)cc1]

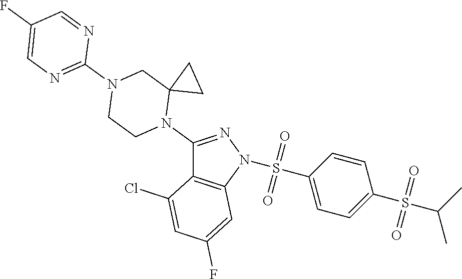 [SMILES: CC(C)S(=O)(=O)c1ccc(S(=O)(=O)n2nc(N3CCN(c4ncc(F)cn4)CC34CC4)c3c(Cl)cc(F)cc32)cc1]

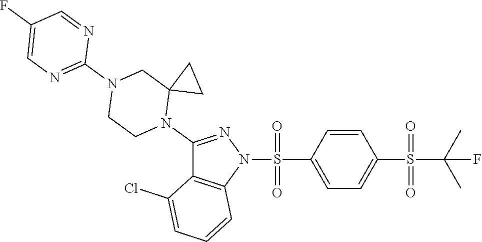 [SMILES: CC(C)(F)S(=O)(=O)c1ccc(S(=O)(=O)n2nc(N3CCN(c4ncc(F)cn4)CC34CC4)c3c(Cl)cccc32)cc1]

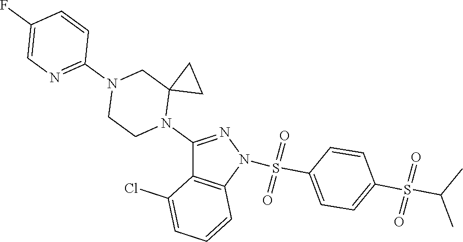 [SMILES: CC(C)S(=O)(=O)c1ccc(S(=O)(=O)n2nc(N3CCN(c4ccc(F)cn4)CC34CC4)c3c(Cl)cccc32)cc1]

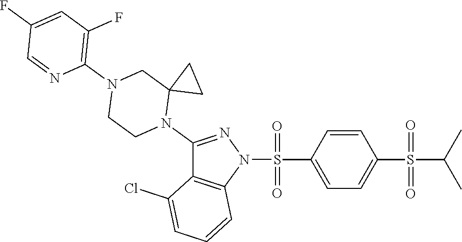 [SMILES: CC(C)S(=O)(=O)c1ccc(S(=O)(=O)n2nc(N3CCN(c4ncc(F)cc4F)CC34CC4)c3c(Cl)cccc32)cc1]

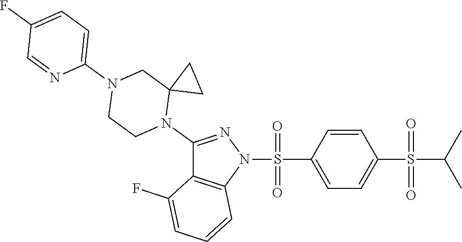 [SMILES: CC(C)S(=O)(=O)c1ccc(S(=O)(=O)n2nc(N3CCN(c4ccc(F)cn4)CC34CC4)c3c(F)cccc32)cc1]

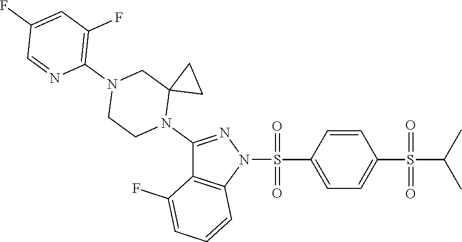 [SMILES: CC(C)S(=O)(=O)c1ccc(S(=O)(=O)n2nc(N3CCN(c4ncc(F)cc4F)CC34CC4)c3c(F)cccc32)cc1]

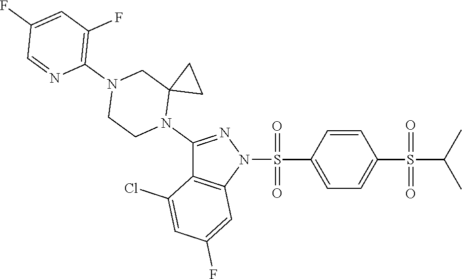 [SMILES: CC(C)S(=O)(=O)c1ccc(S(=O)(=O)n2nc(N3CCN(c4ncc(F)cc4F)CC34CC4)c3c(Cl)cc(F)cc32)cc1]

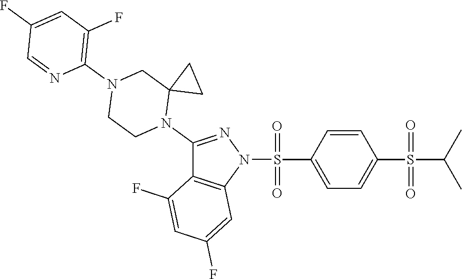 [SMILES: CC(C)S(=O)(=O)c1ccc(S(=O)(=O)n2nc(N3CCN(c4ncc(F)cc4F)CC34CC4)c3c(F)cc(F)cc32)cc1]

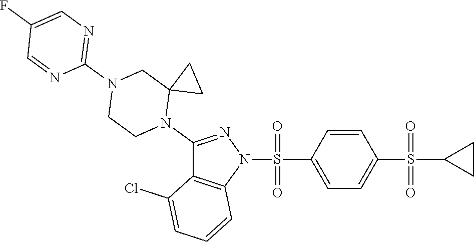 [SMILES: O=S(=O)(c1ccc(S(=O)(=O)n2nc(N3CCN(c4ncc(F)cn4)CC34CC4)c3c(Cl)cccc32)cc1)C1CC1]

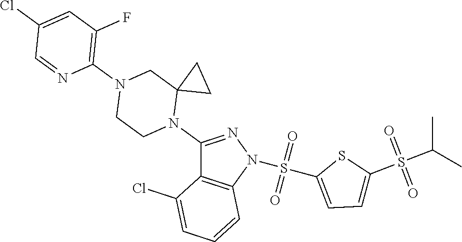 [SMILES: CC(C)S(=O)(=O)c1ccc(S(=O)(=O)n2nc(N3CCN(c4ncc(Cl)cc4F)CC34CC4)c3c(Cl)cccc32)s1]

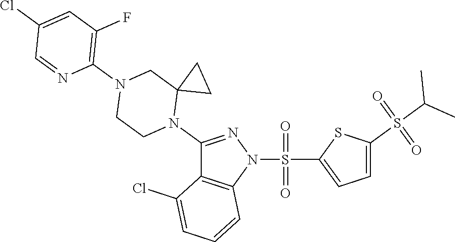 [SMILES: CC(C)S(=O)(=O)c1ccc(S(=O)(=O)n2nc(N3CCN(c4ncc(Cl)cc4F)CC34CC4)c3c(Cl)cccc32)s1]

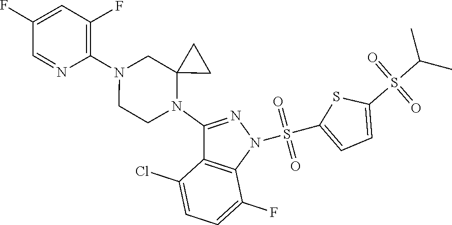 [SMILES: CC(C)S(=O)(=O)c1ccc(S(=O)(=O)n2nc(N3CCN(c4ncc(F)cc4F)CC34CC4)c3c(Cl)ccc(F)c32)s1]

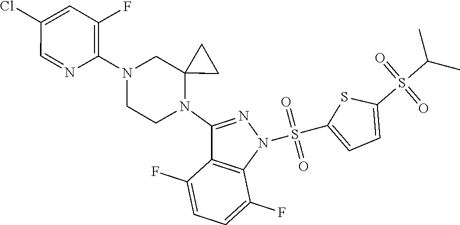 [SMILES: CC(C)S(=O)(=O)c1ccc(S(=O)(=O)n2nc(N3CCN(c4ncc(Cl)cc4F)CC34CC4)c3c(F)ccc(F)c32)s1]

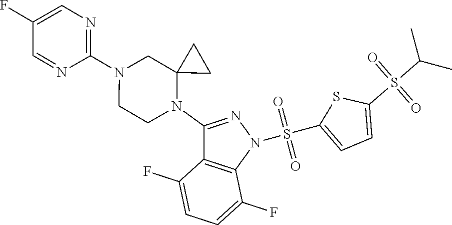 [SMILES: CC(C)S(=O)(=O)c1ccc(S(=O)(=O)n2nc(N3CCN(c4ncc(F)cn4)CC34CC4)c3c(F)ccc(F)c32)s1]

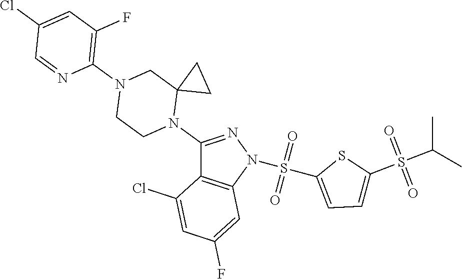 [SMILES: CC(C)S(=O)(=O)c1ccc(S(=O)(=O)n2nc(N3CCN(c4ncc(Cl)cc4F)CC34CC4)c3c(Cl)cc(F)cc32)s1]

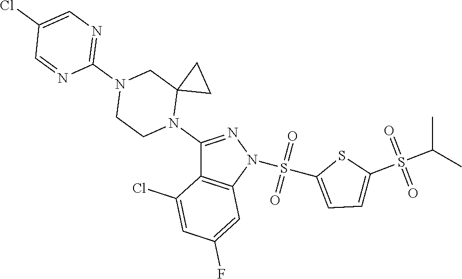 [SMILES: CC(C)S(=O)(=O)c1ccc(S(=O)(=O)n2nc(N3CCN(c4ncc(Cl)cn4)CC34CC4)c3c(Cl)cc(F)cc32)s1]

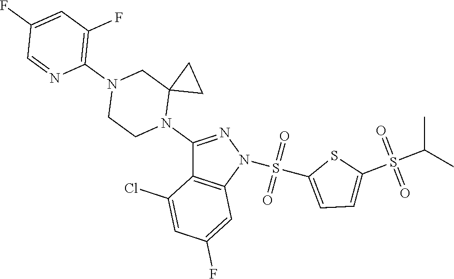 [SMILES: CC(C)S(=O)(=O)c1ccc(S(=O)(=O)n2nc(N3CCN(c4ncc(F)cc4F)CC34CC4)c3c(Cl)cc(F)cc32)s1]

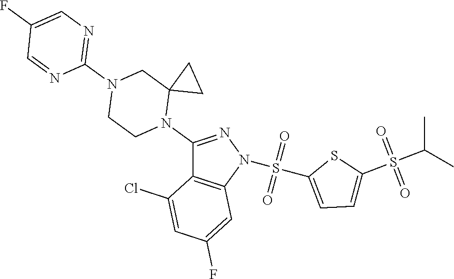 [SMILES: CC(C)S(=O)(=O)c1ccc(S(=O)(=O)n2nc(N3CCN(c4ncc(F)cn4)CC34CC4)c3c(Cl)cc(F)cc32)s1]

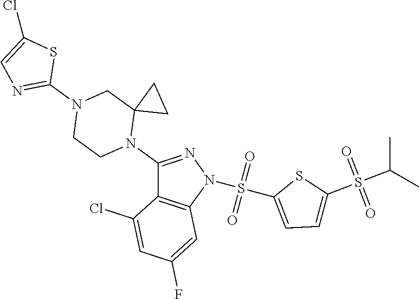 [SMILES: CC(C)S(=O)(=O)c1ccc(S(=O)(=O)n2nc(N3CCN(c4ncc(Cl)s4)CC34CC4)c3c(Cl)cc(F)cc32)s1]

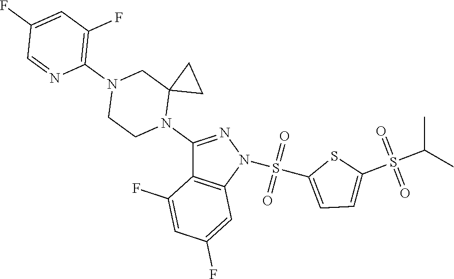 [SMILES: CC(C)S(=O)(=O)c1ccc(S(=O)(=O)n2nc(N3CCN(c4ncc(F)cc4F)CC34CC4)c3c(F)cc(F)cc32)s1]

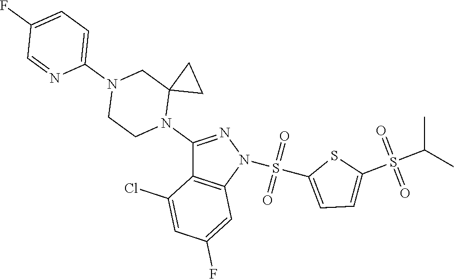 [SMILES: CC(C)S(=O)(=O)c1ccc(S(=O)(=O)n2nc(N3CCN(c4ccc(F)cn4)CC34CC4)c3c(Cl)cc(F)cc32)s1]

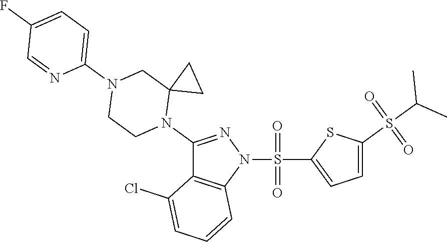 [SMILES: CC(C)S(=O)(=O)c1ccc(S(=O)(=O)n2nc(N3CCN(c4ccc(F)cn4)CC34CC4)c3c(Cl)cccc32)s1]

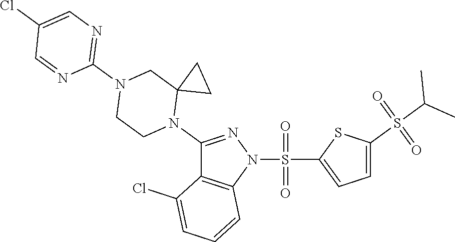 [SMILES: CC(C)S(=O)(=O)c1ccc(S(=O)(=O)n2nc(N3CCN(c4ncc(Cl)cn4)CC34CC4)c3c(Cl)cccc32)s1]

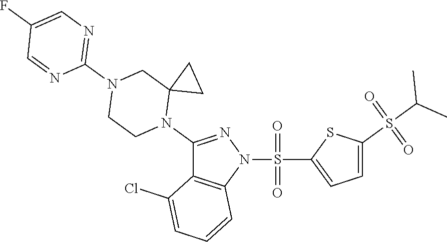 [SMILES: CC(C)S(=O)(=O)c1ccc(S(=O)(=O)n2nc(N3CCN(c4ncc(F)cn4)CC34CC4)c3c(Cl)cccc32)s1]

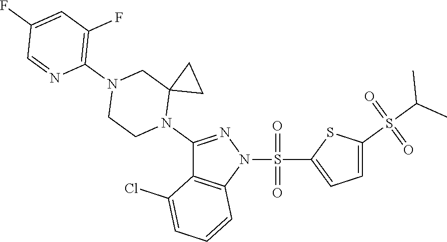 [SMILES: CC(C)S(=O)(=O)c1ccc(S(=O)(=O)n2nc(N3CCN(c4ncc(F)cc4F)CC34CC4)c3c(Cl)cccc32)s1]

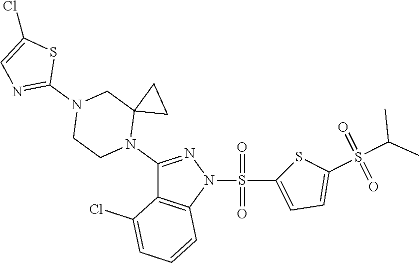 [SMILES: CC(C)S(=O)(=O)c1ccc(S(=O)(=O)n2nc(N3CCN(c4ncc(Cl)s4)CC34CC4)c3c(Cl)cccc32)s1]

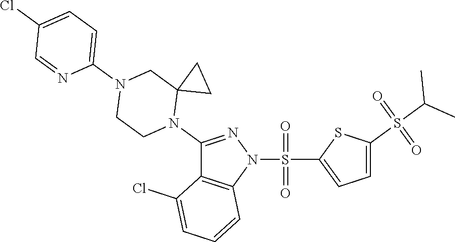 [SMILES: CC(C)S(=O)(=O)c1ccc(S(=O)(=O)n2nc(N3CCN(c4ccc(Cl)cn4)CC34CC4)c3c(Cl)cccc32)s1]

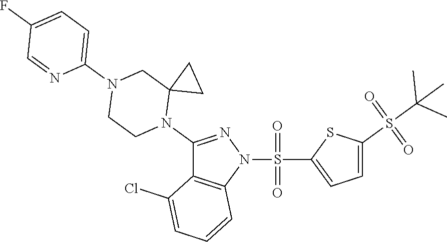 [SMILES: CC(C)(C)S(=O)(=O)c1ccc(S(=O)(=O)n2nc(N3CCN(c4ccc(F)cn4)CC34CC4)c3c(Cl)cccc32)s1]

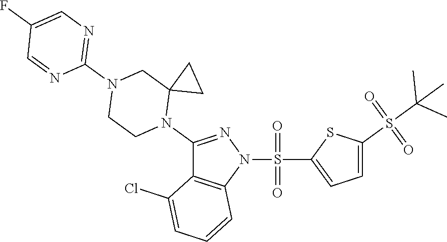 [SMILES: CC(C)(C)S(=O)(=O)c1ccc(S(=O)(=O)n2nc(N3CCN(c4ncc(F)cn4)CC34CC4)c3c(Cl)cccc32)s1]

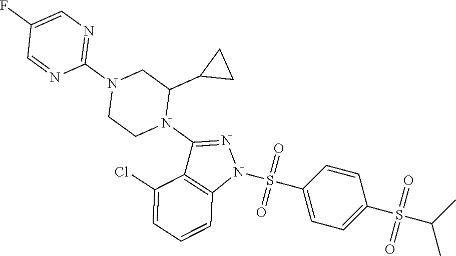 [SMILES: CC(C)S(=O)(=O)c1ccc(S(=O)(=O)n2nc(N3CCN(c4ncc(F)cn4)CC3C3CC3)c3c(Cl)cccc32)cc1]

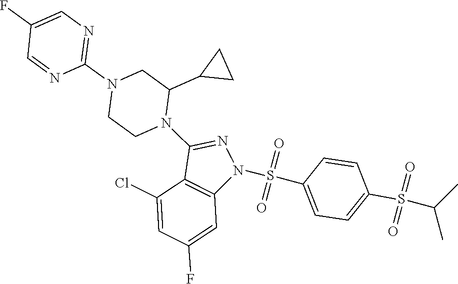 [SMILES: CC(C)S(=O)(=O)c1ccc(S(=O)(=O)n2nc(N3CCN(c4ncc(F)cn4)CC3C3CC3)c3c(Cl)cc(F)cc32)cc1]

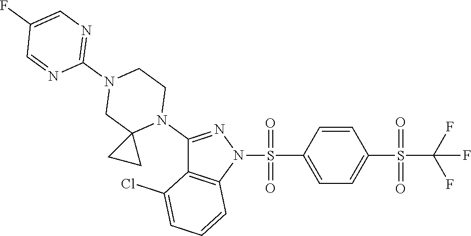 [SMILES: O=S(=O)(c1ccc(S(=O)(=O)C(F)(F)F)cc1)n1nc(N2CCN(c3ncc(F)cn3)CC23CC3)c2c(Cl)cccc21]

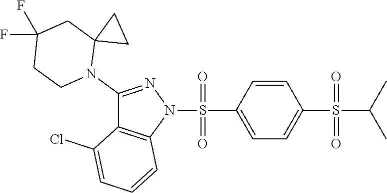 [SMILES: CC(C)S(=O)(=O)c1ccc(S(=O)(=O)n2nc(N3CCC(F)(F)CC34CC4)c3c(Cl)cccc32)cc1]

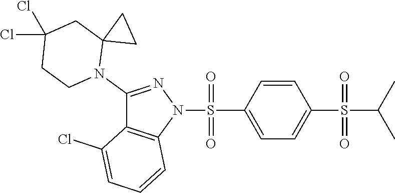 [SMILES: CC(C)S(=O)(=O)c1ccc(S(=O)(=O)n2nc(N3CCC(Cl)(Cl)CC34CC4)c3c(Cl)cccc32)cc1]

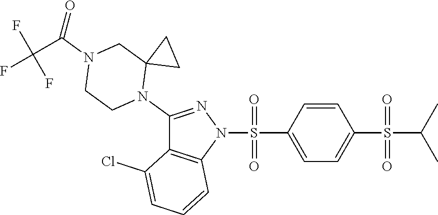 [SMILES: CC(C)S(=O)(=O)c1ccc(S(=O)(=O)n2nc(N3CCN(C(=O)C(F)(F)F)CC34CC4)c3c(Cl)cccc32)cc1]

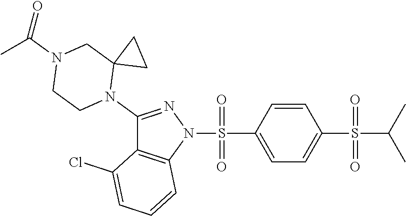 [SMILES: CC(=O)N1CCN(c2nn(S(=O)(=O)c3ccc(S(=O)(=O)C(C)C)cc3)c3cccc(Cl)c23)C2(CC2)C1]

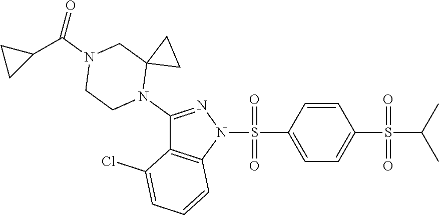 [SMILES: CC(C)S(=O)(=O)c1ccc(S(=O)(=O)n2nc(N3CCN(C(=O)C4CC4)CC34CC4)c3c(Cl)cccc32)cc1]

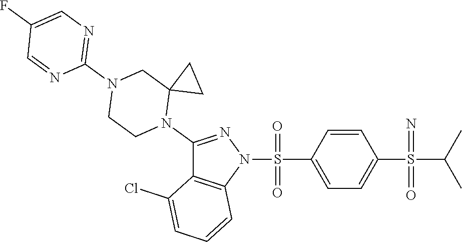 [SMILES: CC(C)S(=N)(=O)c1ccc(S(=O)(=O)n2nc(N3CCN(c4ncc(F)cn4)CC34CC4)c3c(Cl)cccc32)cc1]

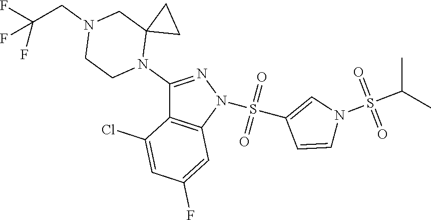 [SMILES: CC(C)S(=O)(=O)n1ccc(S(=O)(=O)n2nc(N3CCN(CC(F)(F)F)CC34CC4)c3c(Cl)cc(F)cc32)c1]

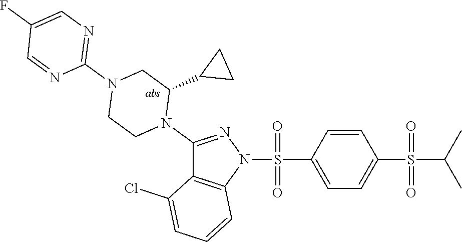 [SMILES: CC(C)S(=O)(=O)c1ccc(S(=O)(=O)n2nc(N3CCN(c4ncc(F)cn4)C[C@@H]3C3CC3)c3c(Cl)cccc32)cc1]

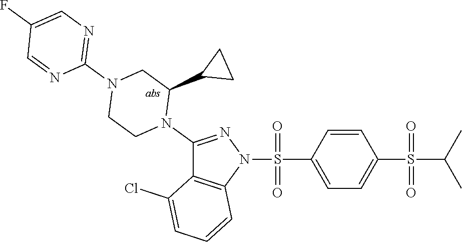 [SMILES: CC(C)S(=O)(=O)c1ccc(S(=O)(=O)n2nc(N3CCN(c4ncc(F)cn4)C[C@H]3C3CC3)c3c(Cl)cccc32)cc1]

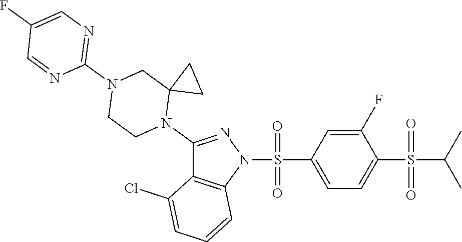 [SMILES: CC(C)S(=O)(=O)c1ccc(S(=O)(=O)n2nc(N3CCN(c4ncc(F)cn4)CC34CC4)c3c(Cl)cccc32)cc1F]

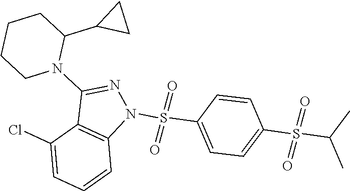 [SMILES: CC(C)S(=O)(=O)c1ccc(S(=O)(=O)n2nc(N3CCCCC3C3CC3)c3c(Cl)cccc32)cc1]

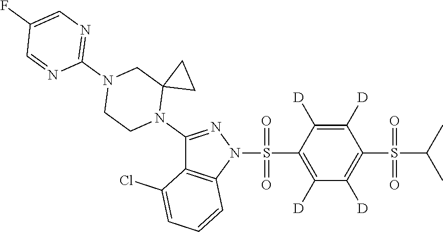 [SMILES: [2H]c1c([2H])c(S(=O)(=O)n2nc(N3CCN(c4ncc(F)cn4)CC34CC4)c3c(Cl)cccc32)c([2H])c([2H])c1S(=O)(=O)C(C)C]

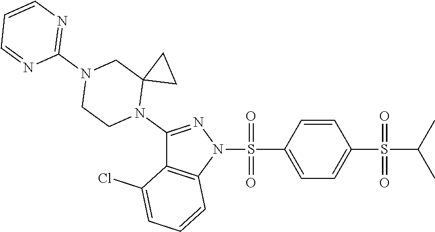 [SMILES: CC(C)S(=O)(=O)c1ccc(S(=O)(=O)n2nc(N3CCN(c4ncccn4)CC34CC4)c3c(Cl)cccc32)cc1]

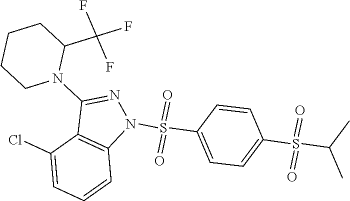 [SMILES: CC(C)S(=O)(=O)c1ccc(S(=O)(=O)n2nc(N3CCCCC3C(F)(F)F)c3c(Cl)cccc32)cc1]

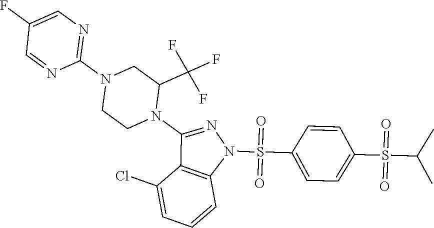 [SMILES: CC(C)S(=O)(=O)c1ccc(S(=O)(=O)n2nc(N3CCN(c4ncc(F)cn4)CC3C(F)(F)F)c3c(Cl)cccc32)cc1]

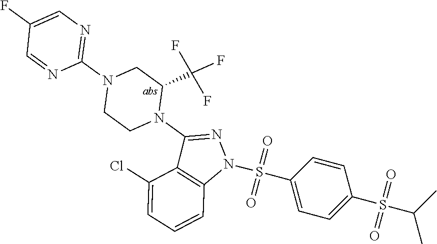 [SMILES: CC(C)S(=O)(=O)c1ccc(S(=O)(=O)n2nc(N3CCN(c4ncc(F)cn4)C[C@@H]3C(F)(F)F)c3c(Cl)cccc32)cc1]

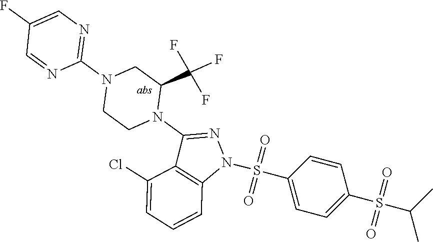 [SMILES: CC(C)S(=O)(=O)c1ccc(S(=O)(=O)n2nc(N3CCN(c4ncc(F)cn4)C[C@H]3C(F)(F)F)c3c(Cl)cccc32)cc1]

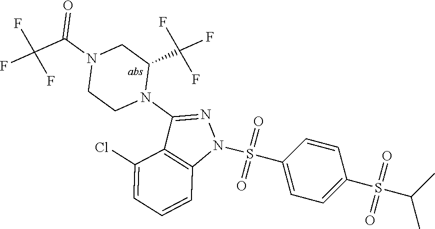 [SMILES: CC(C)S(=O)(=O)c1ccc(S(=O)(=O)n2nc(N3CCN(C(=O)C(F)(F)F)C[C@@H]3C(F)(F)F)c3c(Cl)cccc32)cc1]

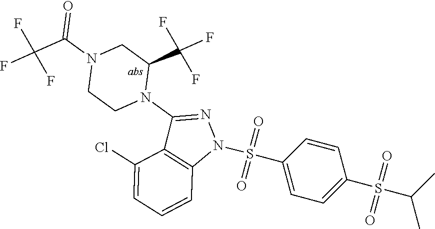 [SMILES: CC(C)S(=O)(=O)c1ccc(S(=O)(=O)n2nc(N3CCN(C(=O)C(F)(F)F)C[C@H]3C(F)(F)F)c3c(Cl)cccc32)cc1]

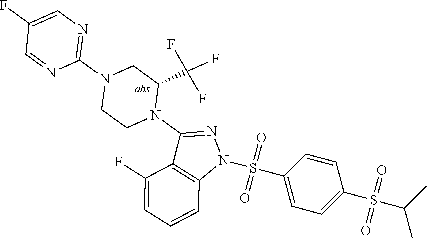 [SMILES: CC(C)S(=O)(=O)c1ccc(S(=O)(=O)n2nc(N3CCN(c4ncc(F)cn4)C[C@@H]3C(F)(F)F)c3c(F)cccc32)cc1]

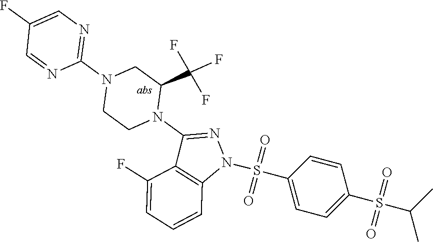 [SMILES: CC(C)S(=O)(=O)c1ccc(S(=O)(=O)n2nc(N3CCN(c4ncc(F)cn4)C[C@H]3C(F)(F)F)c3c(F)cccc32)cc1]

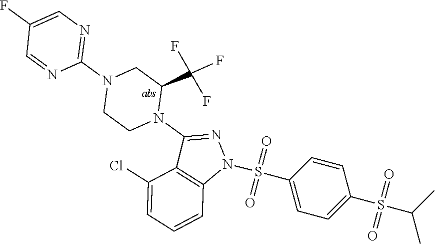 [SMILES: CC(C)S(=O)(=O)c1ccc(S(=O)(=O)n2nc(N3CCN(c4ncc(F)cn4)C[C@H]3C(F)(F)F)c3c(Cl)cccc32)cc1]

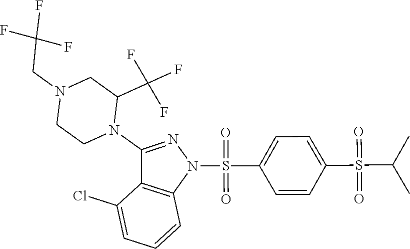 [SMILES: CC(C)S(=O)(=O)c1ccc(S(=O)(=O)n2nc(N3CCN(CC(F)(F)F)CC3C(F)(F)F)c3c(Cl)cccc32)cc1]

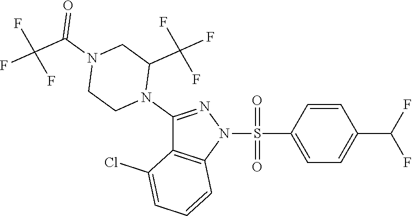 [SMILES: O=C(N1CCN(c2nn(S(=O)(=O)c3ccc(C(F)F)cc3)c3cccc(Cl)c23)C(C(F)(F)F)C1)C(F)(F)F]

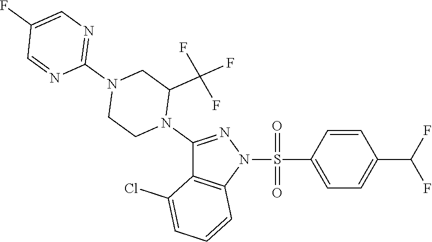 [SMILES: O=S(=O)(c1ccc(C(F)F)cc1)n1nc(N2CCN(c3ncc(F)cn3)CC2C(F)(F)F)c2c(Cl)cccc21]

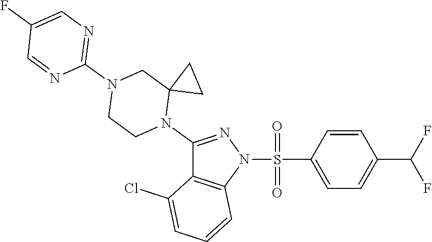 [SMILES: O=S(=O)(c1ccc(C(F)F)cc1)n1nc(N2CCN(c3ncc(F)cn3)CC23CC3)c2c(Cl)cccc21]

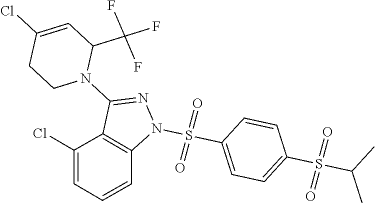 [SMILES: CC(C)S(=O)(=O)c1ccc(S(=O)(=O)n2nc(N3CCC(Cl)=CC3C(F)(F)F)c3c(Cl)cccc32)cc1]

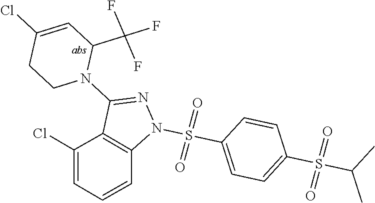 [SMILES: CC(C)S(=O)(=O)c1ccc(S(=O)(=O)n2nc(N3CCC(Cl)=CC3C(F)(F)F)c3c(Cl)cccc32)cc1]

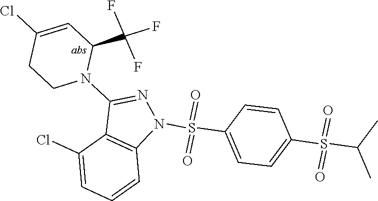 [SMILES: CC(C)S(=O)(=O)c1ccc(S(=O)(=O)n2nc(N3CCC(Cl)=C[C@H]3C(F)(F)F)c3c(Cl)cccc32)cc1]

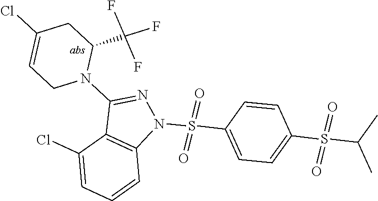 [SMILES: CC(C)S(=O)(=O)c1ccc(S(=O)(=O)n2nc(N3CC=C(Cl)C[C@@H]3C(F)(F)F)c3c(Cl)cccc32)cc1]

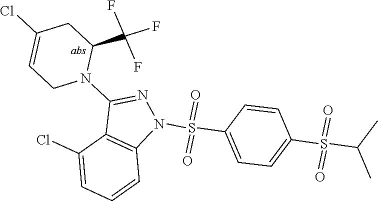 [SMILES: CC(C)S(=O)(=O)c1ccc(S(=O)(=O)n2nc(N3CC=C(Cl)C[C@H]3C(F)(F)F)c3c(Cl)cccc32)cc1]

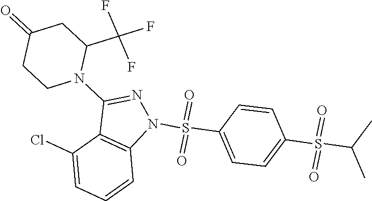 [SMILES: CC(C)S(=O)(=O)c1ccc(S(=O)(=O)n2nc(N3CCC(=O)CC3C(F)(F)F)c3c(Cl)cccc32)cc1]

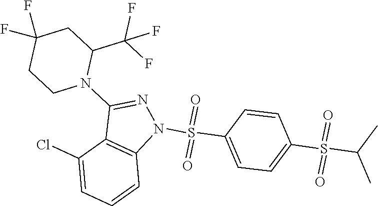 [SMILES: CC(C)S(=O)(=O)c1ccc(S(=O)(=O)n2nc(N3CCC(F)(F)CC3C(F)(F)F)c3c(Cl)cccc32)cc1]

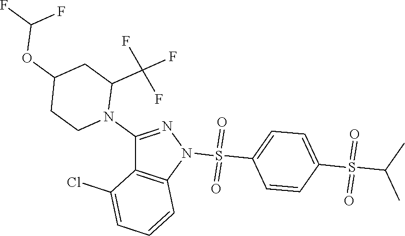 [SMILES: CC(C)S(=O)(=O)c1ccc(S(=O)(=O)n2nc(N3CCC(OC(F)F)CC3C(F)(F)F)c3c(Cl)cccc32)cc1]

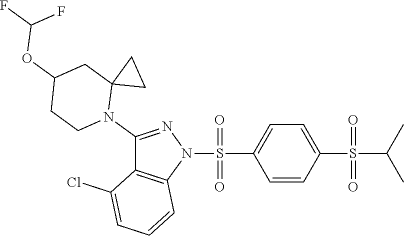 [SMILES: CC(C)S(=O)(=O)c1ccc(S(=O)(=O)n2nc(N3CCC(OC(F)F)CC34CC4)c3c(Cl)cccc32)cc1]

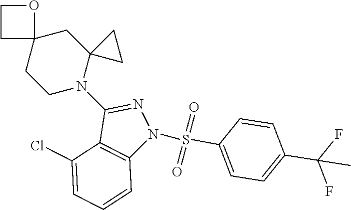 [SMILES: CC(F)(F)c1ccc(S(=O)(=O)n2nc(N3CCC4(CCO4)CC34CC4)c3c(Cl)cccc32)cc1]

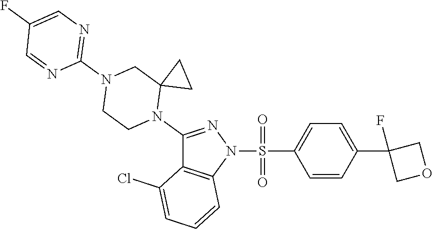 [SMILES: O=S(=O)(c1ccc(C2(F)COC2)cc1)n1nc(N2CCN(c3ncc(F)cn3)CC23CC3)c2c(Cl)cccc21]

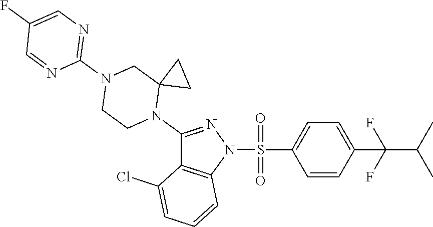 [SMILES: CC(C)C(F)(F)c1ccc(S(=O)(=O)n2nc(N3CCN(c4ncc(F)cn4)CC34CC4)c3c(Cl)cccc32)cc1]

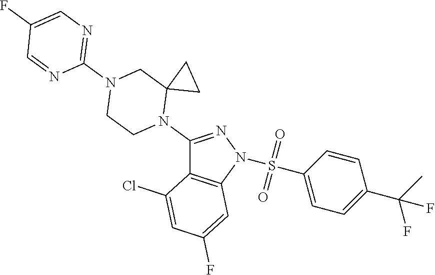 [SMILES: CC(F)(F)c1ccc(S(=O)(=O)n2nc(N3CCN(c4ncc(F)cn4)CC34CC4)c3c(Cl)cc(F)cc32)cc1]

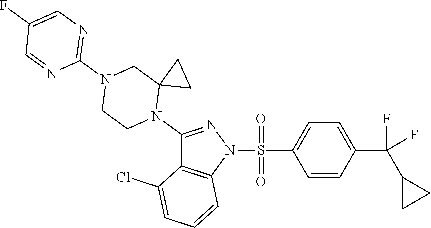 [SMILES: O=S(=O)(c1ccc(C(F)(F)C2CC2)cc1)n1nc(N2CCN(c3ncc(F)cn3)CC23CC3)c2c(Cl)cccc21]

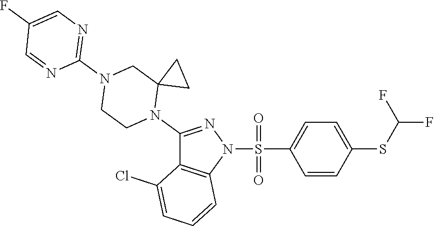 [SMILES: O=S(=O)(c1ccc(SC(F)F)cc1)n1nc(N2CCN(c3ncc(F)cn3)CC23CC3)c2c(Cl)cccc21]

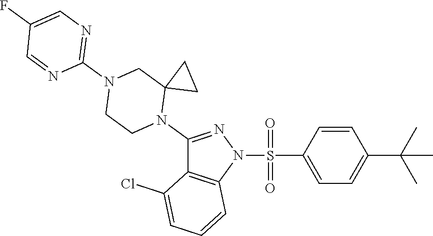 [SMILES: CC(C)(C)c1ccc(S(=O)(=O)n2nc(N3CCN(c4ncc(F)cn4)CC34CC4)c3c(Cl)cccc32)cc1]

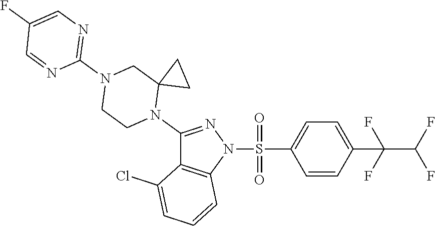 [SMILES: O=S(=O)(c1ccc(C(F)(F)C(F)F)cc1)n1nc(N2CCN(c3ncc(F)cn3)CC23CC3)c2c(Cl)cccc21]

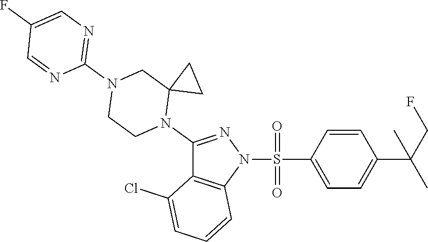 [SMILES: CC(C)(CF)c1ccc(S(=O)(=O)n2nc(N3CCN(c4ncc(F)cn4)CC34CC4)c3c(Cl)cccc32)cc1]

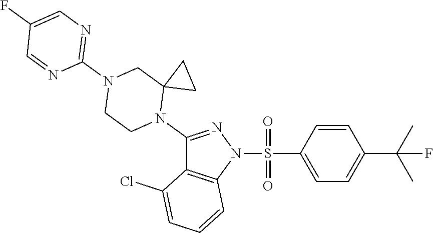 [SMILES: CC(C)(F)c1ccc(S(=O)(=O)n2nc(N3CCN(c4ncc(F)cn4)CC34CC4)c3c(Cl)cccc32)cc1]

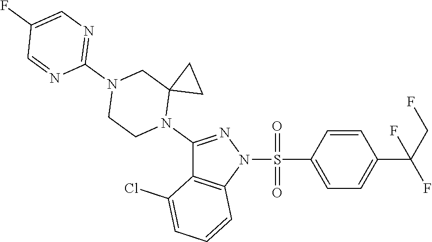 [SMILES: O=S(=O)(c1ccc(C(F)(F)CF)cc1)n1nc(N2CCN(c3ncc(F)cn3)CC23CC3)c2c(Cl)cccc21]

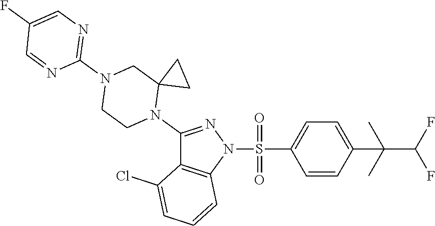 [SMILES: CC(C)(c1ccc(S(=O)(=O)n2nc(N3CCN(c4ncc(F)cn4)CC34CC4)c3c(Cl)cccc32)cc1)C(F)F]

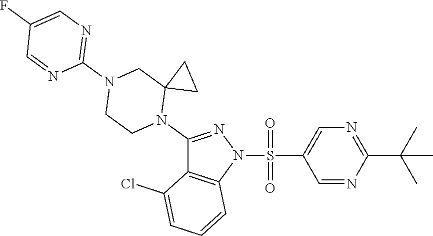 [SMILES: CC(C)(C)c1ncc(S(=O)(=O)n2nc(N3CCN(c4ncc(F)cn4)CC34CC4)c3c(Cl)cccc32)cn1]

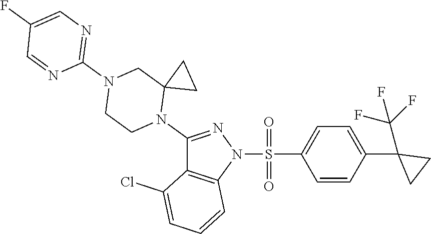 [SMILES: O=S(=O)(c1ccc(C2(C(F)(F)F)CC2)cc1)n1nc(N2CCN(c3ncc(F)cn3)CC23CC3)c2c(Cl)cccc21]

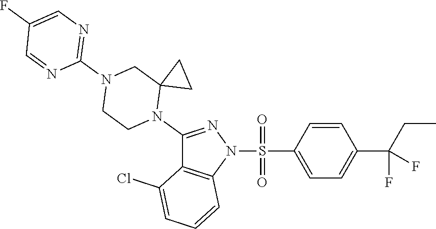 [SMILES: CCC(F)(F)c1ccc(S(=O)(=O)n2nc(N3CCN(c4ncc(F)cn4)CC34CC4)c3c(Cl)cccc32)cc1]

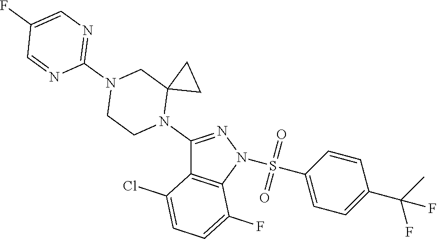 [SMILES: CC(F)(F)c1ccc(S(=O)(=O)n2nc(N3CCN(c4ncc(F)cn4)CC34CC4)c3c(Cl)ccc(F)c32)cc1]

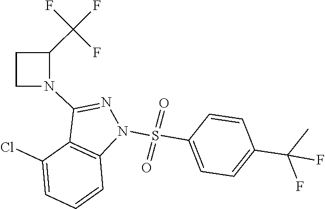 [SMILES: CC(F)(F)c1ccc(S(=O)(=O)n2nc(N3CCC3C(F)(F)F)c3c(Cl)cccc32)cc1]

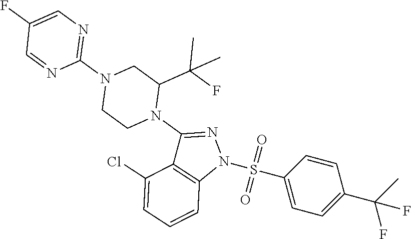 [SMILES: CC(F)(F)c1ccc(S(=O)(=O)n2nc(N3CCN(c4ncc(F)cn4)CC3C(C)(C)F)c3c(Cl)cccc32)cc1]

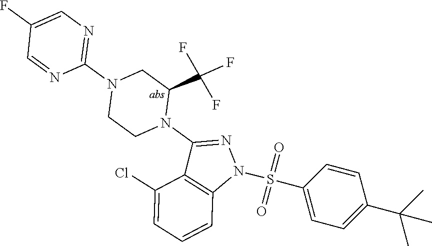 [SMILES: CC(C)(C)c1ccc(S(=O)(=O)n2nc(N3CCN(c4ncc(F)cn4)C[C@H]3C(F)(F)F)c3c(Cl)cccc32)cc1]

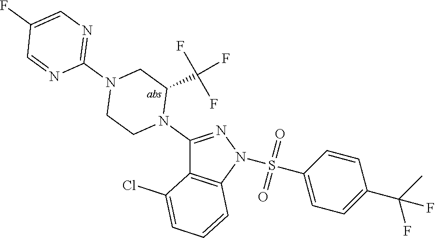 [SMILES: CC(F)(F)c1ccc(S(=O)(=O)n2nc(N3CCN(c4ncc(F)cn4)C[C@@H]3C(F)(F)F)c3c(Cl)cccc32)cc1]

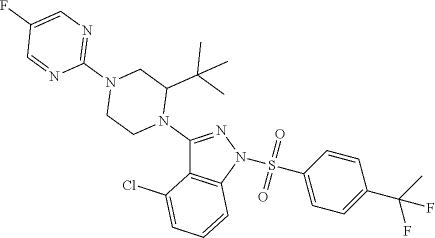 [SMILES: CC(F)(F)c1ccc(S(=O)(=O)n2nc(N3CCN(c4ncc(F)cn4)CC3C(C)(C)C)c3c(Cl)cccc32)cc1]

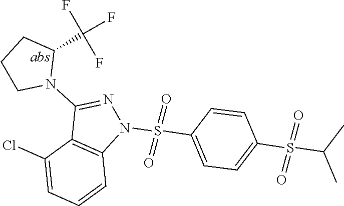 [SMILES: CC(C)S(=O)(=O)c1ccc(S(=O)(=O)n2nc(N3CCC[C@@H]3C(F)(F)F)c3c(Cl)cccc32)cc1]

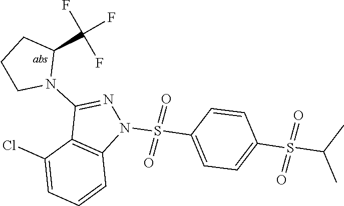 [SMILES: CC(C)S(=O)(=O)c1ccc(S(=O)(=O)n2nc(N3CCC[C@H]3C(F)(F)F)c3c(Cl)cccc32)cc1]

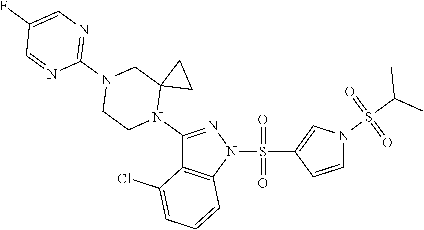 [SMILES: CC(C)S(=O)(=O)n1ccc(S(=O)(=O)n2nc(N3CCN(c4ncc(F)cn4)CC34CC4)c3c(Cl)cccc32)c1]

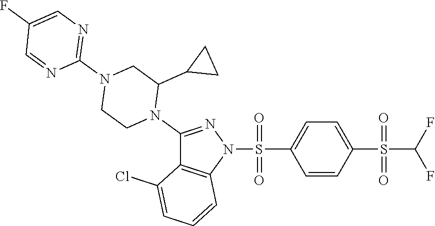 [SMILES: O=S(=O)(c1ccc(S(=O)(=O)n2nc(N3CCN(c4ncc(F)cn4)CC3C3CC3)c3c(Cl)cccc32)cc1)C(F)F]

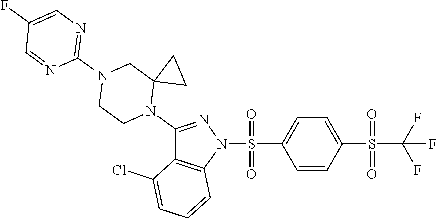 [SMILES: O=S(=O)(c1ccc(S(=O)(=O)C(F)(F)F)cc1)n1nc(N2CCN(c3ncc(F)cn3)CC23CC3)c2c(Cl)cccc21]

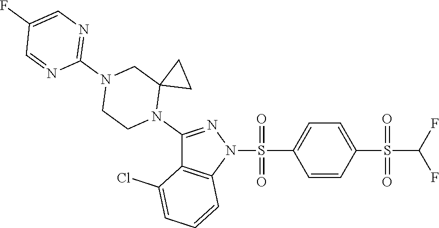 [SMILES: O=S(=O)(c1ccc(S(=O)(=O)n2nc(N3CCN(c4ncc(F)cn4)CC34CC4)c3c(Cl)cccc32)cc1)C(F)F]

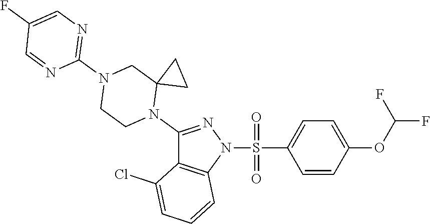 [SMILES: O=S(=O)(c1ccc(OC(F)F)cc1)n1nc(N2CCN(c3ncc(F)cn3)CC23CC3)c2c(Cl)cccc21]

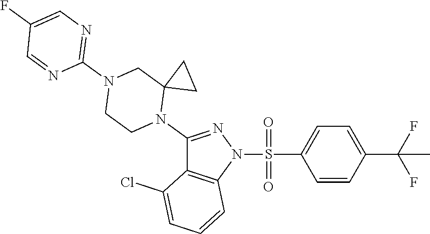 [SMILES: CC(F)(F)c1ccc(S(=O)(=O)n2nc(N3CCN(c4ncc(F)cn4)CC34CC4)c3c(Cl)cccc32)cc1]

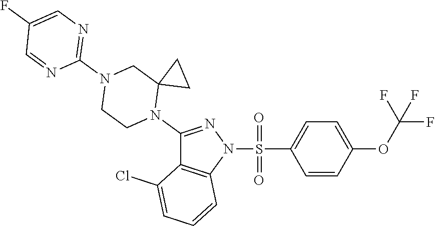 [SMILES: O=S(=O)(c1ccc(OC(F)(F)F)cc1)n1nc(N2CCN(c3ncc(F)cn3)CC23CC3)c2c(Cl)cccc21]

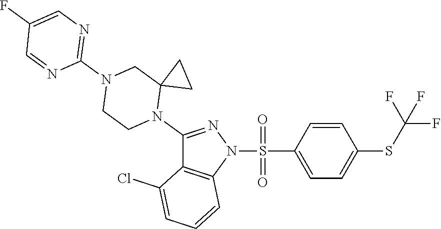 [SMILES: O=S(=O)(c1ccc(SC(F)(F)F)cc1)n1nc(N2CCN(c3ncc(F)cn3)CC23CC3)c2c(Cl)cccc21]

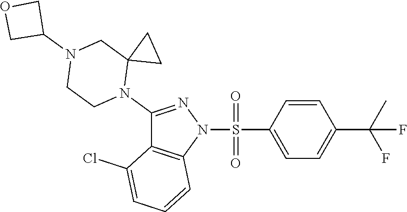 [SMILES: CC(F)(F)c1ccc(S(=O)(=O)n2nc(N3CCN(C4COC4)CC34CC4)c3c(Cl)cccc32)cc1]

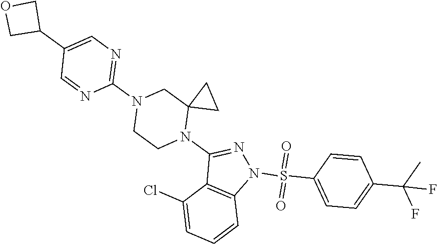 [SMILES: CC(F)(F)c1ccc(S(=O)(=O)n2nc(N3CCN(c4ncc(C5COC5)cn4)CC34CC4)c3c(Cl)cccc32)cc1]

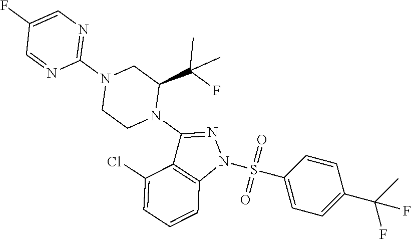 [SMILES: CC(F)(F)c1ccc(S(=O)(=O)n2nc(N3CCN(c4ncc(F)cn4)C[C@H]3C(C)(C)F)c3c(Cl)cccc32)cc1]

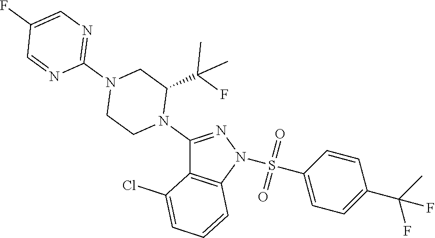 [SMILES: CC(F)(F)c1ccc(S(=O)(=O)n2nc(N3CCN(c4ncc(F)cn4)C[C@@H]3C(C)(C)F)c3c(Cl)cccc32)cc1]

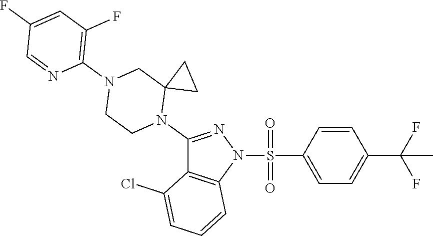 [SMILES: CC(F)(F)c1ccc(S(=O)(=O)n2nc(N3CCN(c4ncc(F)cc4F)CC34CC4)c3c(Cl)cccc32)cc1]

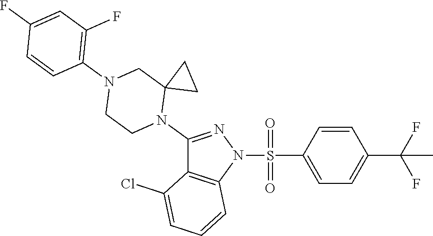 [SMILES: CC(F)(F)c1ccc(S(=O)(=O)n2nc(N3CCN(c4ccc(F)cc4F)CC34CC4)c3c(Cl)cccc32)cc1]

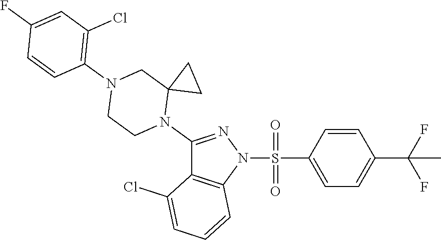 [SMILES: CC(F)(F)c1ccc(S(=O)(=O)n2nc(N3CCN(c4ccc(F)cc4Cl)CC34CC4)c3c(Cl)cccc32)cc1]

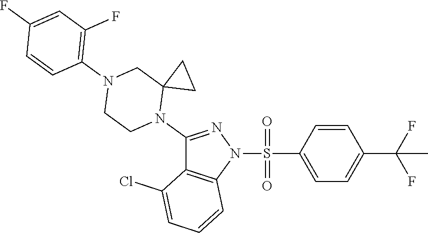 [SMILES: CC(F)(F)c1ccc(S(=O)(=O)n2nc(N3CCN(c4ccc(F)cc4F)CC34CC4)c3c(Cl)cccc32)cc1]

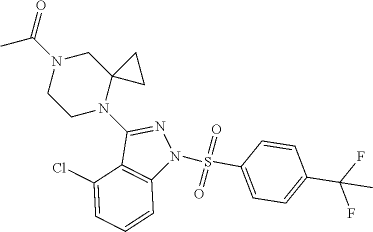 [SMILES: CC(=O)N1CCN(c2nn(S(=O)(=O)c3ccc(C(C)(F)F)cc3)c3cccc(Cl)c23)C2(CC2)C1]

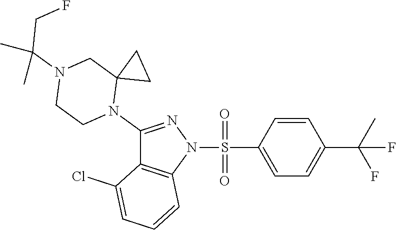 [SMILES: CC(F)(F)c1ccc(S(=O)(=O)n2nc(N3CCN(C(C)(C)CF)CC34CC4)c3c(Cl)cccc32)cc1]

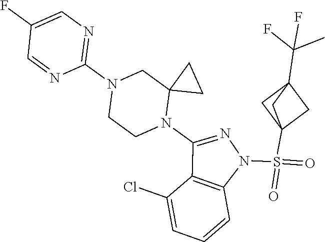 [SMILES: CC(F)(F)C12CC(S(=O)(=O)n3nc(N4CCN(c5ncc(F)cn5)CC45CC5)c4c(Cl)cccc43)(C1)C2]

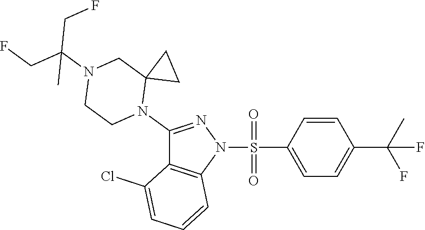 [SMILES: CC(F)(F)c1ccc(S(=O)(=O)n2nc(N3CCN(C(C)(CF)CF)CC34CC4)c3c(Cl)cccc32)cc1]

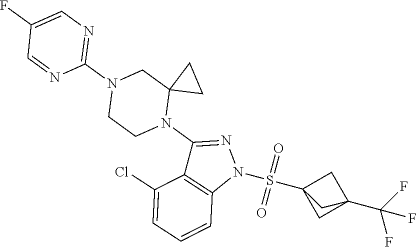 [SMILES: O=S(=O)(n1nc(N2CCN(c3ncc(F)cn3)CC23CC3)c2c(Cl)cccc21)C12CC(C(F)(F)F)(C1)C2]

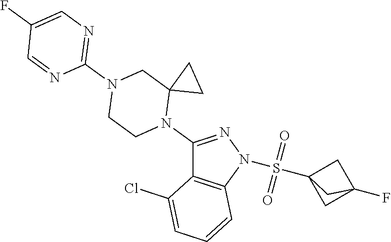 [SMILES: O=S(=O)(n1nc(N2CCN(c3ncc(F)cn3)CC23CC3)c2c(Cl)cccc21)C12CC(F)(C1)C2]

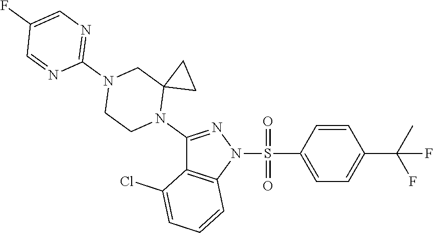 [SMILES: CC(F)(F)c1ccc(S(=O)(=O)n2nc(N3CCN(c4ncc(F)cn4)CC34CC4)c3c(Cl)cccc32)cc1]

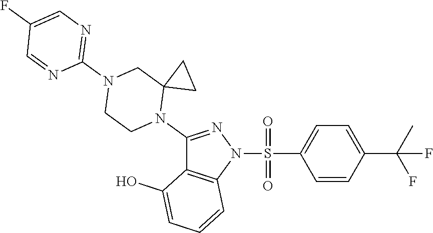 [SMILES: CC(F)(F)c1ccc(S(=O)(=O)n2nc(N3CCN(c4ncc(F)cn4)CC34CC4)c3c(O)cccc32)cc1]

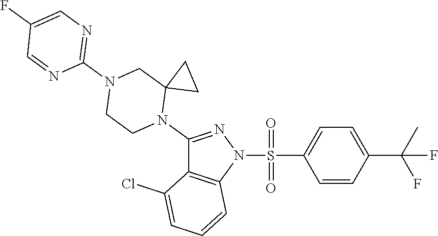 [SMILES: CC(F)(F)c1ccc(S(=O)(=O)n2nc(N3CCN(c4ncc(F)cn4)CC34CC4)c3c(Cl)cccc32)cc1]

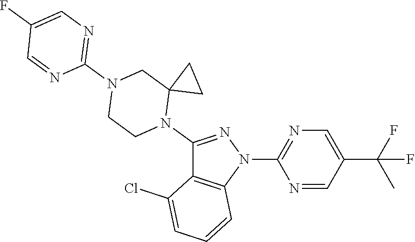 [SMILES: CC(F)(F)c1cnc(-n2nc(N3CCN(c4ncc(F)cn4)CC34CC4)c3c(Cl)cccc32)nc1]

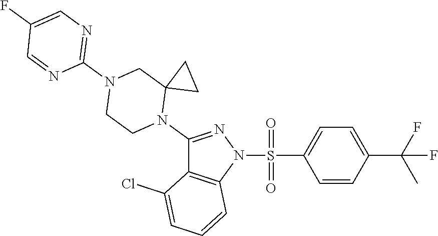 [SMILES: CC(F)(F)c1ccc(S(=O)(=O)n2nc(N3CCN(c4ncc(F)cn4)CC34CC4)c3c(Cl)cccc32)cc1]

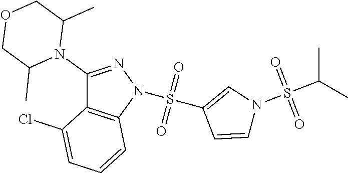 [SMILES: CC1COCC(C)N1c1nn(S(=O)(=O)c2ccn(S(=O)(=O)C(C)C)c2)c2cccc(Cl)c12]

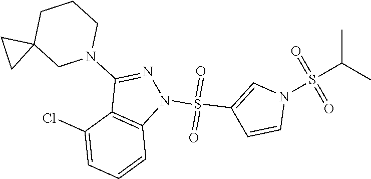 [SMILES: CC(C)S(=O)(=O)n1ccc(S(=O)(=O)n2nc(N3CCCC4(CC4)C3)c3c(Cl)cccc32)c1]

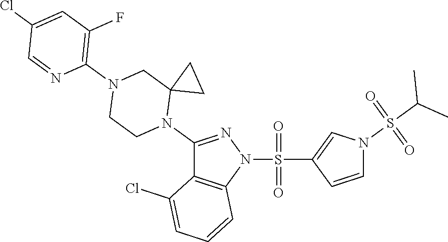 [SMILES: CC(C)S(=O)(=O)n1ccc(S(=O)(=O)n2nc(N3CCN(c4ncc(Cl)cc4F)CC34CC4)c3c(Cl)cccc32)c1]

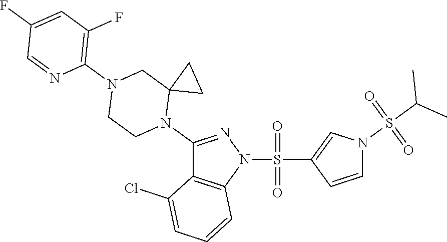 [SMILES: CC(C)S(=O)(=O)n1ccc(S(=O)(=O)n2nc(N3CCN(c4ncc(F)cc4F)CC34CC4)c3c(Cl)cccc32)c1]

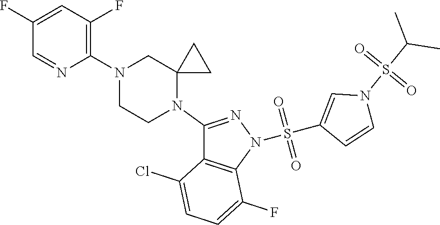 [SMILES: CC(C)S(=O)(=O)n1ccc(S(=O)(=O)n2nc(N3CCN(c4ncc(F)cc4F)CC34CC4)c3c(Cl)ccc(F)c32)c1]

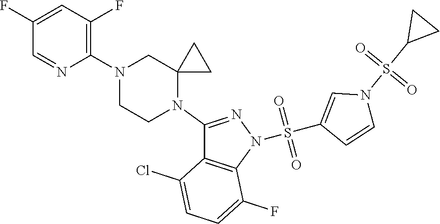 [SMILES: O=S(=O)(c1ccn(S(=O)(=O)C2CC2)c1)n1nc(N2CCN(c3ncc(F)cc3F)CC23CC3)c2c(Cl)ccc(F)c21]

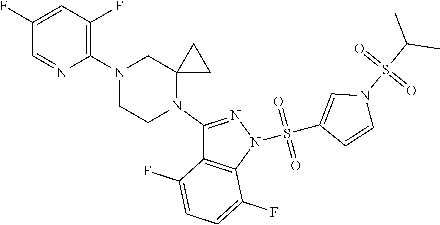 [SMILES: CC(C)S(=O)(=O)n1ccc(S(=O)(=O)n2nc(N3CCN(c4ncc(F)cc4F)CC34CC4)c3c(F)ccc(F)c32)c1]

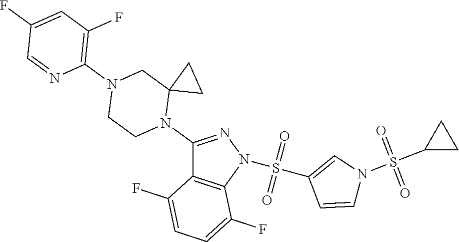 [SMILES: O=S(=O)(c1ccn(S(=O)(=O)C2CC2)c1)n1nc(N2CCN(c3ncc(F)cc3F)CC23CC3)c2c(F)ccc(F)c21]

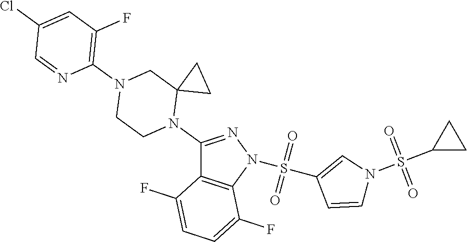 [SMILES: O=S(=O)(c1ccn(S(=O)(=O)C2CC2)c1)n1nc(N2CCN(c3ncc(Cl)cc3F)CC23CC3)c2c(F)ccc(F)c21]

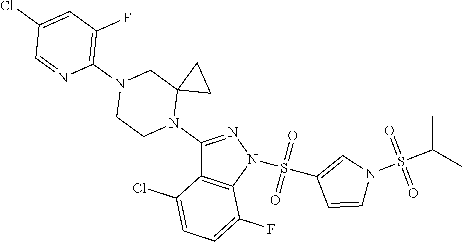 [SMILES: CC(C)S(=O)(=O)n1ccc(S(=O)(=O)n2nc(N3CCN(c4ncc(Cl)cc4F)CC34CC4)c3c(Cl)ccc(F)c32)c1]

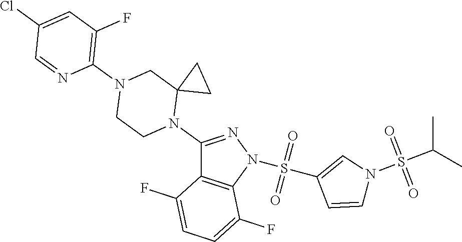 [SMILES: CC(C)S(=O)(=O)n1ccc(S(=O)(=O)n2nc(N3CCN(c4ncc(Cl)cc4F)CC34CC4)c3c(F)ccc(F)c32)c1]

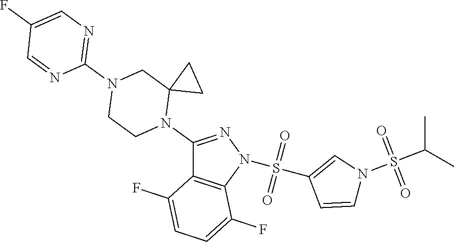 [SMILES: CC(C)S(=O)(=O)n1ccc(S(=O)(=O)n2nc(N3CCN(c4ncc(F)cn4)CC34CC4)c3c(F)ccc(F)c32)c1]

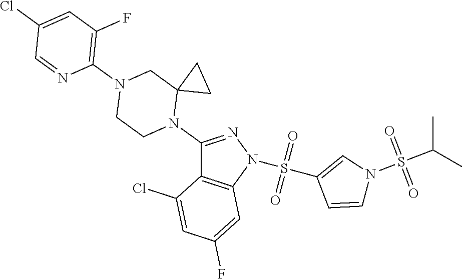 [SMILES: CC(C)S(=O)(=O)n1ccc(S(=O)(=O)n2nc(N3CCN(c4ncc(Cl)cc4F)CC34CC4)c3c(Cl)cc(F)cc32)c1]

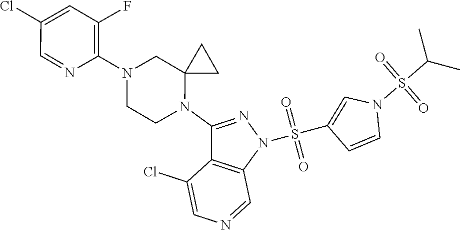 [SMILES: CC(C)S(=O)(=O)n1ccc(S(=O)(=O)n2nc(N3CCN(c4ncc(Cl)cc4F)CC34CC4)c3c(Cl)cncc32)c1]

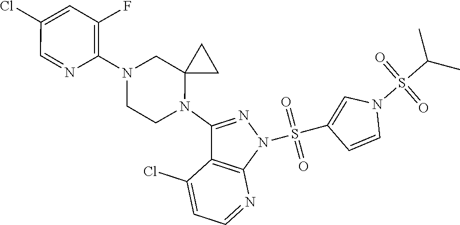 [SMILES: CC(C)S(=O)(=O)n1ccc(S(=O)(=O)n2nc(N3CCN(c4ncc(Cl)cc4F)CC34CC4)c3c(Cl)ccnc32)c1]

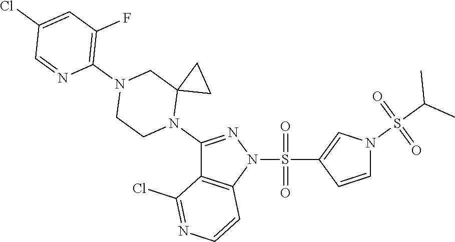 [SMILES: CC(C)S(=O)(=O)n1ccc(S(=O)(=O)n2nc(N3CCN(c4ncc(Cl)cc4F)CC34CC4)c3c(Cl)nccc32)c1]

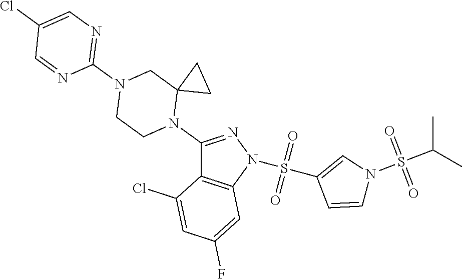 [SMILES: CC(C)S(=O)(=O)n1ccc(S(=O)(=O)n2nc(N3CCN(c4ncc(Cl)cn4)CC34CC4)c3c(Cl)cc(F)cc32)c1]

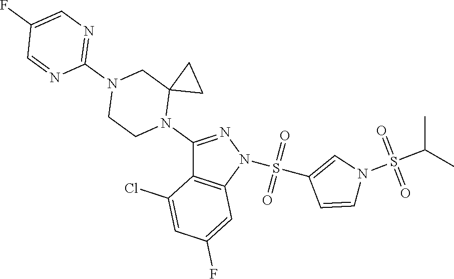 [SMILES: CC(C)S(=O)(=O)n1ccc(S(=O)(=O)n2nc(N3CCN(c4ncc(F)cn4)CC34CC4)c3c(Cl)cc(F)cc32)c1]

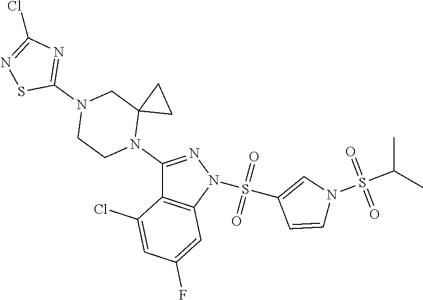 [SMILES: CC(C)S(=O)(=O)n1ccc(S(=O)(=O)n2nc(N3CCN(c4nc(Cl)ns4)CC34CC4)c3c(Cl)cc(F)cc32)c1]

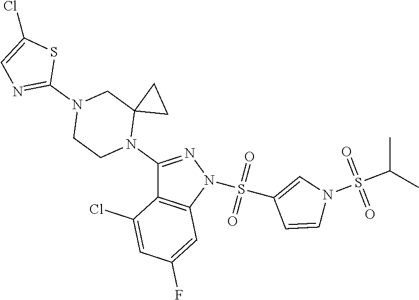 [SMILES: CC(C)S(=O)(=O)n1ccc(S(=O)(=O)n2nc(N3CCN(c4ncc(Cl)s4)CC34CC4)c3c(Cl)cc(F)cc32)c1]

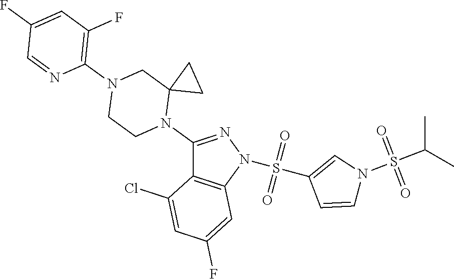 [SMILES: CC(C)S(=O)(=O)n1ccc(S(=O)(=O)n2nc(N3CCN(c4ncc(F)cc4F)CC34CC4)c3c(Cl)cc(F)cc32)c1]

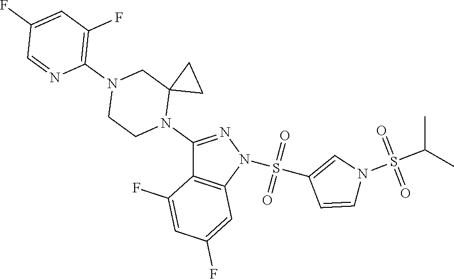 [SMILES: CC(C)S(=O)(=O)n1ccc(S(=O)(=O)n2nc(N3CCN(c4ncc(F)cc4F)CC34CC4)c3c(F)cc(F)cc32)c1]

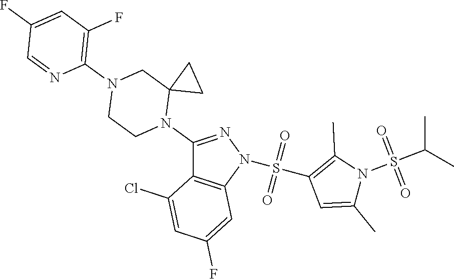 [SMILES: Cc1cc(S(=O)(=O)n2nc(N3CCN(c4ncc(F)cc4F)CC34CC4)c3c(Cl)cc(F)cc32)c(C)n1S(=O)(=O)C(C)C]

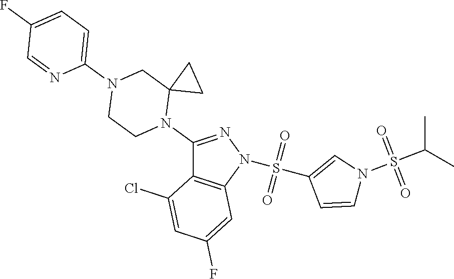 [SMILES: CC(C)S(=O)(=O)n1ccc(S(=O)(=O)n2nc(N3CCN(c4ccc(F)cn4)CC34CC4)c3c(Cl)cc(F)cc32)c1]

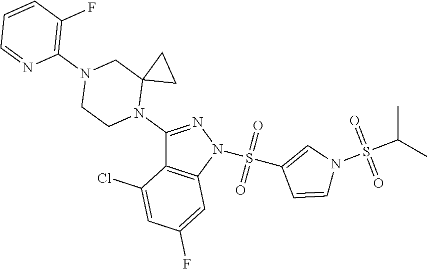 [SMILES: CC(C)S(=O)(=O)n1ccc(S(=O)(=O)n2nc(N3CCN(c4ncccc4F)CC34CC4)c3c(Cl)cc(F)cc32)c1]

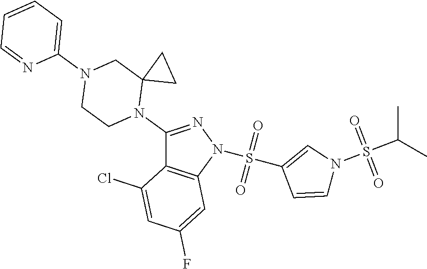 [SMILES: CC(C)S(=O)(=O)n1ccc(S(=O)(=O)n2nc(N3CCN(c4ccccn4)CC34CC4)c3c(Cl)cc(F)cc32)c1]

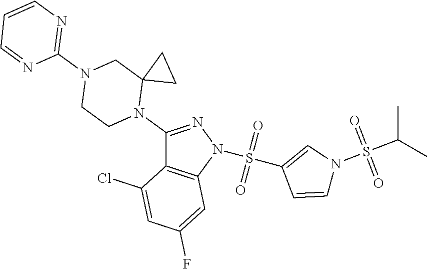 [SMILES: CC(C)S(=O)(=O)n1ccc(S(=O)(=O)n2nc(N3CCN(c4ncccn4)CC34CC4)c3c(Cl)cc(F)cc32)c1]

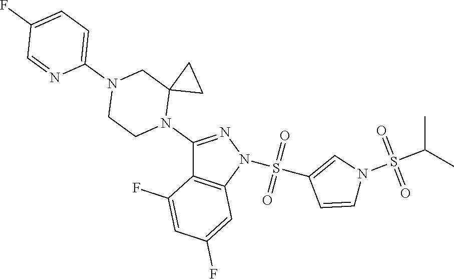 [SMILES: CC(C)S(=O)(=O)n1ccc(S(=O)(=O)n2nc(N3CCN(c4ccc(F)cn4)CC34CC4)c3c(F)cc(F)cc32)c1]

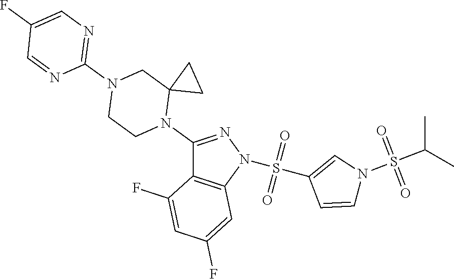 [SMILES: CC(C)S(=O)(=O)n1ccc(S(=O)(=O)n2nc(N3CCN(c4ncc(F)cn4)CC34CC4)c3c(F)cc(F)cc32)c1]

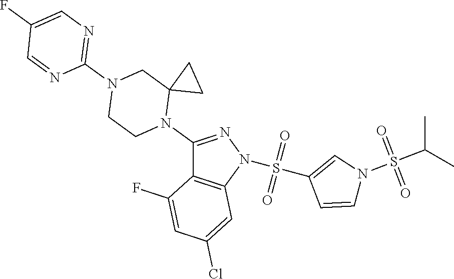 [SMILES: CC(C)S(=O)(=O)n1ccc(S(=O)(=O)n2nc(N3CCN(c4ncc(F)cn4)CC34CC4)c3c(F)cc(Cl)cc32)c1]

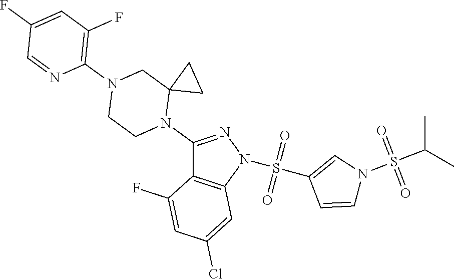 [SMILES: CC(C)S(=O)(=O)n1ccc(S(=O)(=O)n2nc(N3CCN(c4ncc(F)cc4F)CC34CC4)c3c(F)cc(Cl)cc32)c1]

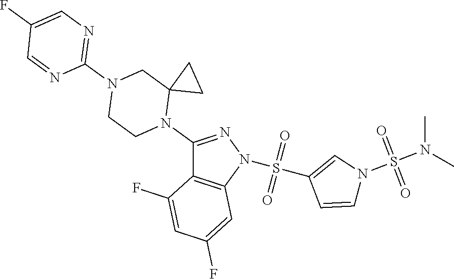 [SMILES: CN(C)S(=O)(=O)n1ccc(S(=O)(=O)n2nc(N3CCN(c4ncc(F)cn4)CC34CC4)c3c(F)cc(F)cc32)c1]

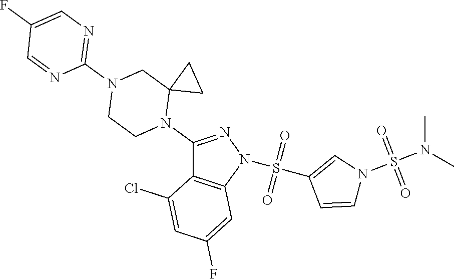 [SMILES: CN(C)S(=O)(=O)n1ccc(S(=O)(=O)n2nc(N3CCN(c4ncc(F)cn4)CC34CC4)c3c(Cl)cc(F)cc32)c1]

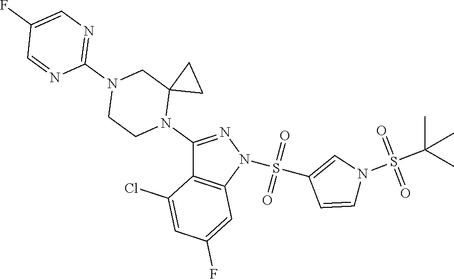 [SMILES: CC(C)(C)S(=O)(=O)n1ccc(S(=O)(=O)n2nc(N3CCN(c4ncc(F)cn4)CC34CC4)c3c(Cl)cc(F)cc32)c1]

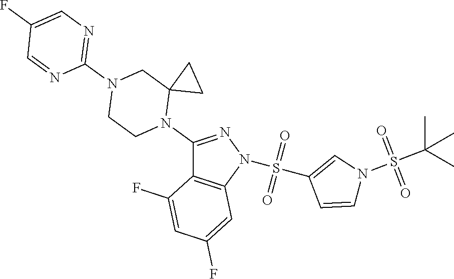 [SMILES: CC(C)(C)S(=O)(=O)n1ccc(S(=O)(=O)n2nc(N3CCN(c4ncc(F)cn4)CC34CC4)c3c(F)cc(F)cc32)c1]

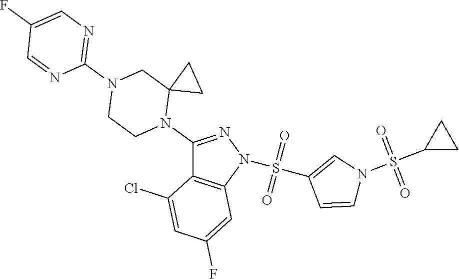 [SMILES: O=S(=O)(c1ccn(S(=O)(=O)C2CC2)c1)n1nc(N2CCN(c3ncc(F)cn3)CC23CC3)c2c(Cl)cc(F)cc21]

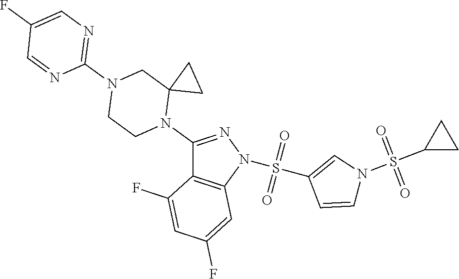 [SMILES: O=S(=O)(c1ccn(S(=O)(=O)C2CC2)c1)n1nc(N2CCN(c3ncc(F)cn3)CC23CC3)c2c(F)cc(F)cc21]

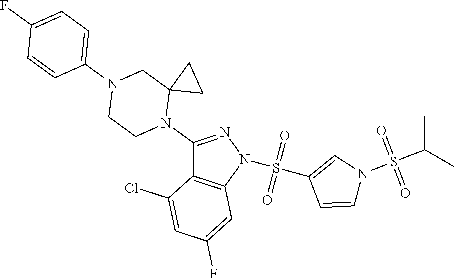 [SMILES: CC(C)S(=O)(=O)n1ccc(S(=O)(=O)n2nc(N3CCN(c4ccc(F)cc4)CC34CC4)c3c(Cl)cc(F)cc32)c1]

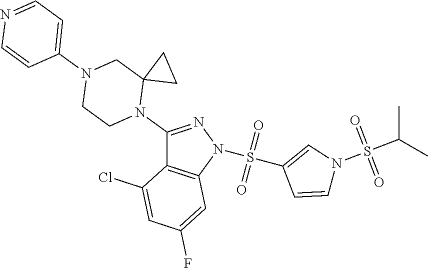 [SMILES: CC(C)S(=O)(=O)n1ccc(S(=O)(=O)n2nc(N3CCN(c4ccncc4)CC34CC4)c3c(Cl)cc(F)cc32)c1]

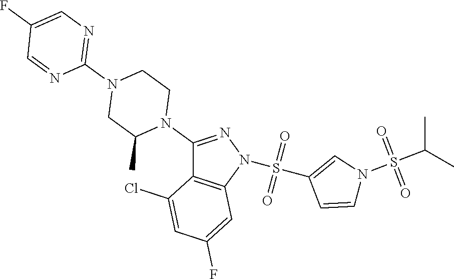 [SMILES: CC(C)S(=O)(=O)n1ccc(S(=O)(=O)n2nc(N3CCN(c4ncc(F)cn4)C[C@@H]3C)c3c(Cl)cc(F)cc32)c1]

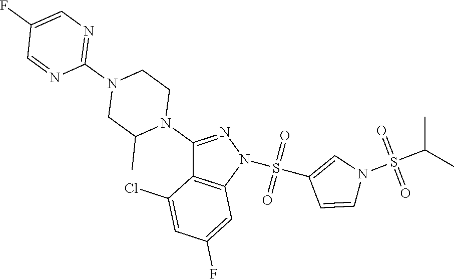 [SMILES: CC1CN(c2ncc(F)cn2)CCN1c1nn(S(=O)(=O)c2ccn(S(=O)(=O)C(C)C)c2)c2cc(F)cc(Cl)c12]

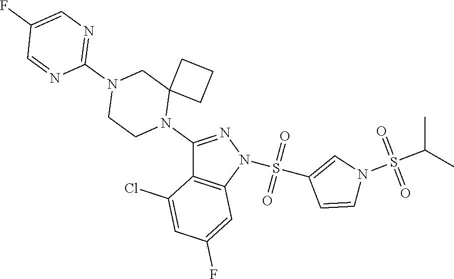 [SMILES: CC(C)S(=O)(=O)n1ccc(S(=O)(=O)n2nc(N3CCN(c4ncc(F)cn4)CC34CCC4)c3c(Cl)cc(F)cc32)c1]

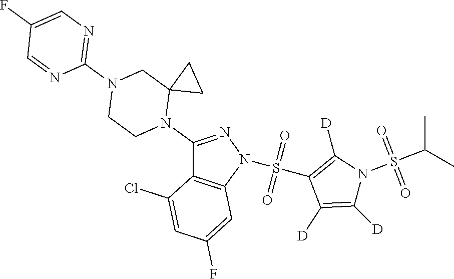 [SMILES: [2H]c1c(S(=O)(=O)n2nc(N3CCN(c4ncc(F)cn4)CC34CC4)c3c(Cl)cc(F)cc32)c([2H])n(S(=O)(=O)C(C)C)c1[2H]]

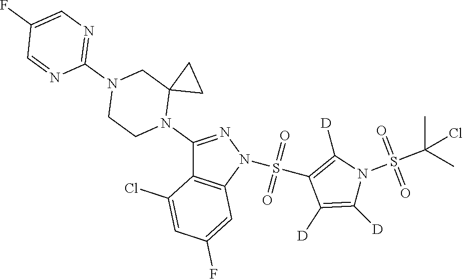 [SMILES: [2H]c1c(S(=O)(=O)n2nc(N3CCN(c4ncc(F)cn4)CC34CC4)c3c(Cl)cc(F)cc32)c([2H])n(S(=O)(=O)C(C)(C)Cl)c1[2H]]

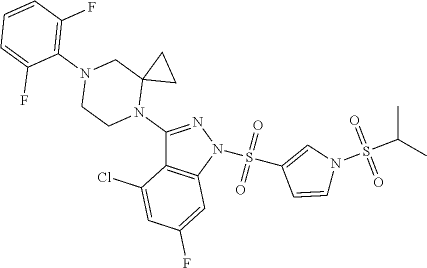 [SMILES: CC(C)S(=O)(=O)n1ccc(S(=O)(=O)n2nc(N3CCN(c4c(F)cccc4F)CC34CC4)c3c(Cl)cc(F)cc32)c1]

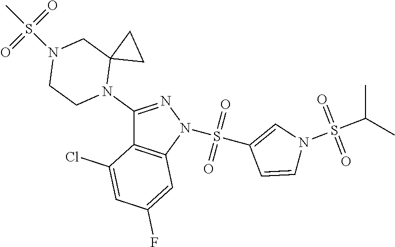 [SMILES: CC(C)S(=O)(=O)n1ccc(S(=O)(=O)n2nc(N3CCN(S(C)(=O)=O)CC34CC4)c3c(Cl)cc(F)cc32)c1]

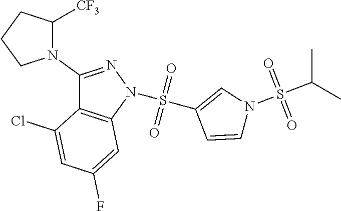 [SMILES: CC(C)S(=O)(=O)n1ccc(S(=O)(=O)n2nc(N3CCCC3C(F)(F)F)c3c(Cl)cc(F)cc32)c1]

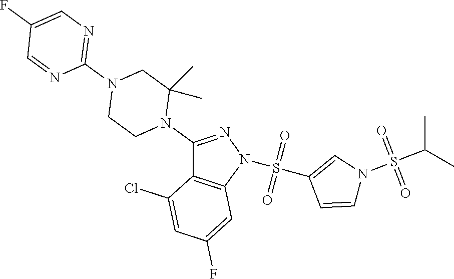 [SMILES: CC(C)S(=O)(=O)n1ccc(S(=O)(=O)n2nc(N3CCN(c4ncc(F)cn4)CC3(C)C)c3c(Cl)cc(F)cc32)c1]

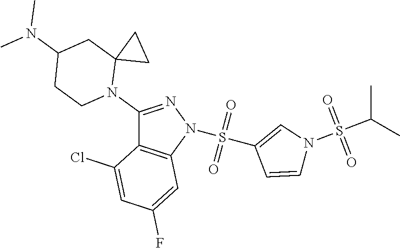 [SMILES: CC(C)S(=O)(=O)n1ccc(S(=O)(=O)n2nc(N3CCC(N(C)C)CC34CC4)c3c(Cl)cc(F)cc32)c1]

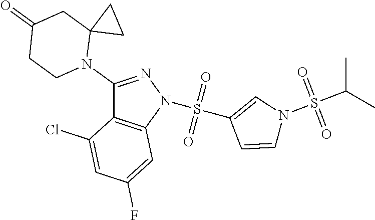 [SMILES: CC(C)S(=O)(=O)n1ccc(S(=O)(=O)n2nc(N3CCC(=O)CC34CC4)c3c(Cl)cc(F)cc32)c1]

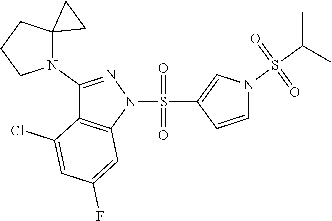 [SMILES: CC(C)S(=O)(=O)n1ccc(S(=O)(=O)n2nc(N3CCCC34CC4)c3c(Cl)cc(F)cc32)c1]

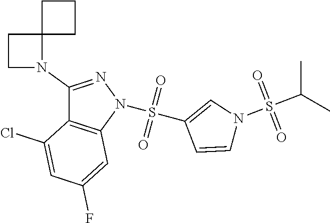 [SMILES: CC(C)S(=O)(=O)n1ccc(S(=O)(=O)n2nc(N3CCC34CCC4)c3c(Cl)cc(F)cc32)c1]

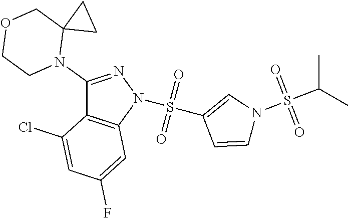 [SMILES: CC(C)S(=O)(=O)n1ccc(S(=O)(=O)n2nc(N3CCOCC34CC4)c3c(Cl)cc(F)cc32)c1]

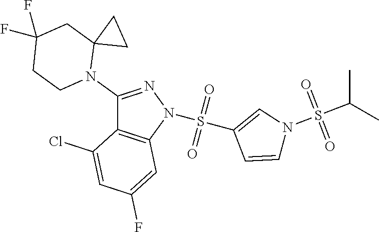 [SMILES: CC(C)S(=O)(=O)n1ccc(S(=O)(=O)n2nc(N3CCC(F)(F)CC34CC4)c3c(Cl)cc(F)cc32)c1]

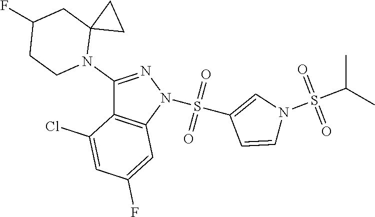 [SMILES: CC(C)S(=O)(=O)n1ccc(S(=O)(=O)n2nc(N3CCC(F)CC34CC4)c3c(Cl)cc(F)cc32)c1]

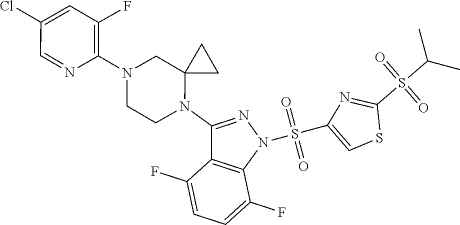 [SMILES: CC(C)S(=O)(=O)c1nc(S(=O)(=O)n2nc(N3CCN(c4ncc(Cl)cc4F)CC34CC4)c3c(F)ccc(F)c32)cs1]

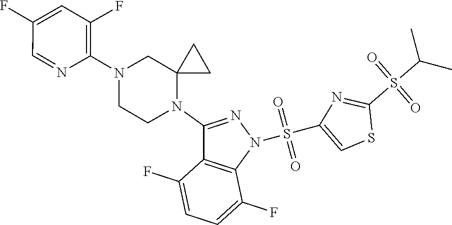 [SMILES: CC(C)S(=O)(=O)c1nc(S(=O)(=O)n2nc(N3CCN(c4ncc(F)cc4F)CC34CC4)c3c(F)ccc(F)c32)cs1]

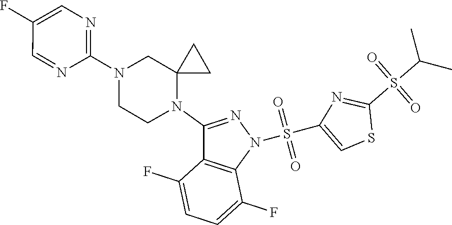 [SMILES: CC(C)S(=O)(=O)c1nc(S(=O)(=O)n2nc(N3CCN(c4ncc(F)cn4)CC34CC4)c3c(F)ccc(F)c32)cs1]

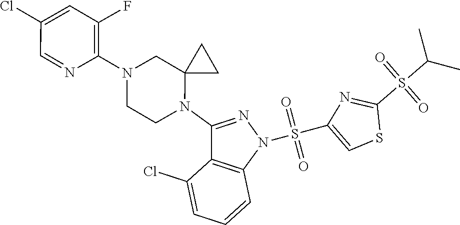 [SMILES: CC(C)S(=O)(=O)c1nc(S(=O)(=O)n2nc(N3CCN(c4ncc(Cl)cc4F)CC34CC4)c3c(Cl)cccc32)cs1]

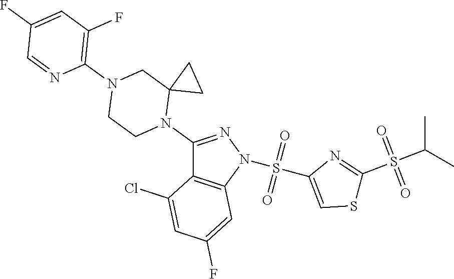 [SMILES: CC(C)S(=O)(=O)c1nc(S(=O)(=O)n2nc(N3CCN(c4ncc(F)cc4F)CC34CC4)c3c(Cl)cc(F)cc32)cs1]

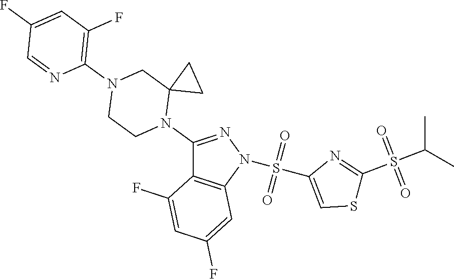 [SMILES: CC(C)S(=O)(=O)c1nc(S(=O)(=O)n2nc(N3CCN(c4ncc(F)cc4F)CC34CC4)c3c(F)cc(F)cc32)cs1]

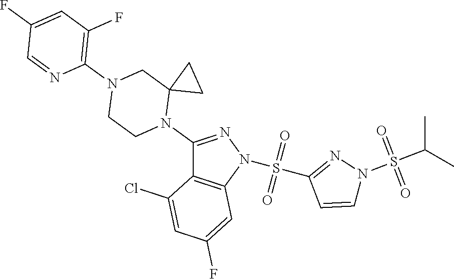 [SMILES: CC(C)S(=O)(=O)n1ccc(S(=O)(=O)n2nc(N3CCN(c4ncc(F)cc4F)CC34CC4)c3c(Cl)cc(F)cc32)n1]

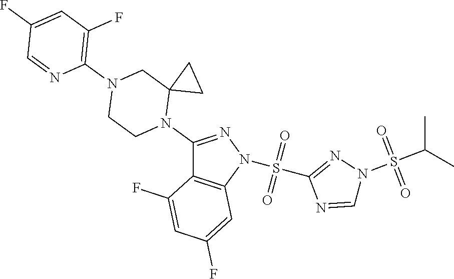 [SMILES: CC(C)S(=O)(=O)n1cnc(S(=O)(=O)n2nc(N3CCN(c4ncc(F)cc4F)CC34CC4)c3c(F)cc(F)cc32)n1]

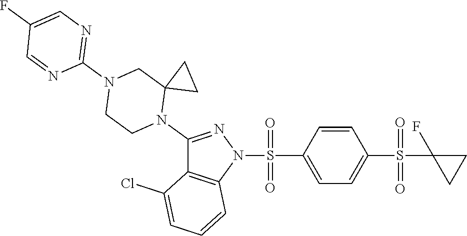 [SMILES: O=S(=O)(c1ccc(S(=O)(=O)C2(F)CC2)cc1)n1nc(N2CCN(c3ncc(F)cn3)CC23CC3)c2c(Cl)cccc21]

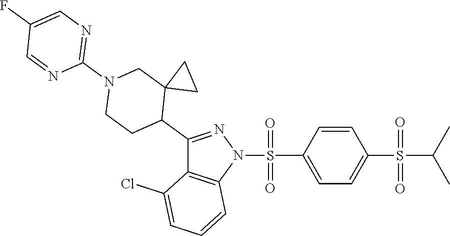 [SMILES: CC(C)S(=O)(=O)c1ccc(S(=O)(=O)n2nc(C3CCN(c4ncc(F)cn4)CC34CC4)c3c(Cl)cccc32)cc1]

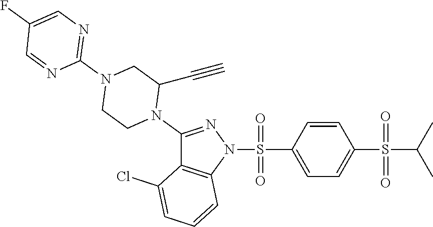 [SMILES: C#CC1CN(c2ncc(F)cn2)CCN1c1nn(S(=O)(=O)c2ccc(S(=O)(=O)C(C)C)cc2)c2cccc(Cl)c12]

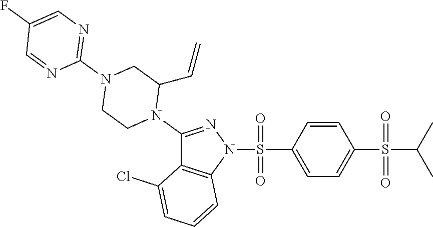 [SMILES: C=CC1CN(c2ncc(F)cn2)CCN1c1nn(S(=O)(=O)c2ccc(S(=O)(=O)C(C)C)cc2)c2cccc(Cl)c12]

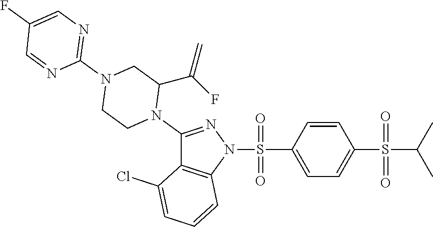 [SMILES: C=C(F)C1CN(c2ncc(F)cn2)CCN1c1nn(S(=O)(=O)c2ccc(S(=O)(=O)C(C)C)cc2)c2cccc(Cl)c12]

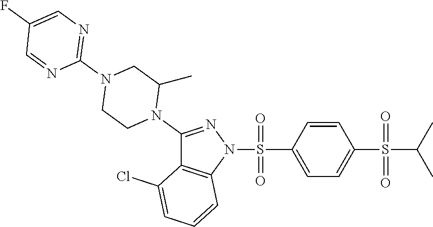 [SMILES: CC1CN(c2ncc(F)cn2)CCN1c1nn(S(=O)(=O)c2ccc(S(=O)(=O)C(C)C)cc2)c2cccc(Cl)c12]

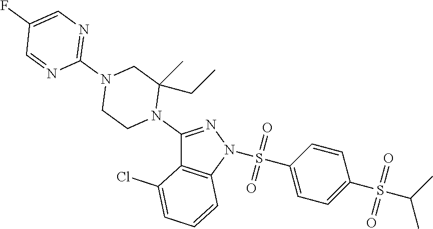 [SMILES: CCC1(C)CN(c2ncc(F)cn2)CCN1c1nn(S(=O)(=O)c2ccc(S(=O)(=O)C(C)C)cc2)c2cccc(Cl)c12]

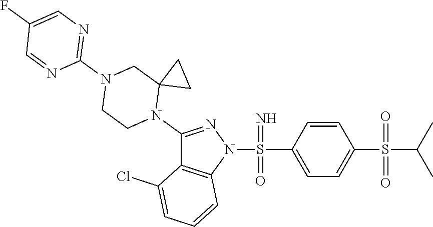 [SMILES: CC(C)S(=O)(=O)c1ccc(S(=N)(=O)n2nc(N3CCN(c4ncc(F)cn4)CC34CC4)c3c(Cl)cccc32)cc1]

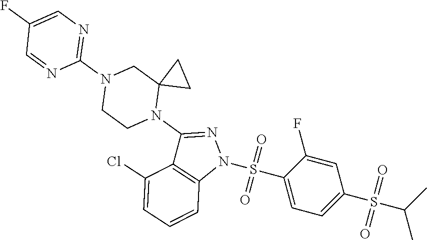 [SMILES: CC(C)S(=O)(=O)c1ccc(S(=O)(=O)n2nc(N3CCN(c4ncc(F)cn4)CC34CC4)c3c(Cl)cccc32)c(F)c1]

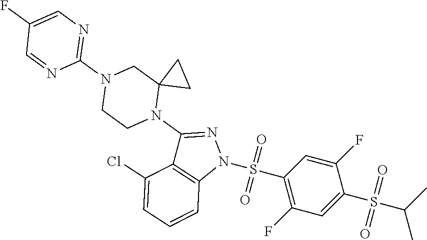 [SMILES: CC(C)S(=O)(=O)c1cc(F)c(S(=O)(=O)n2nc(N3CCN(c4ncc(F)cn4)CC34CC4)c3c(Cl)cccc32)cc1F]

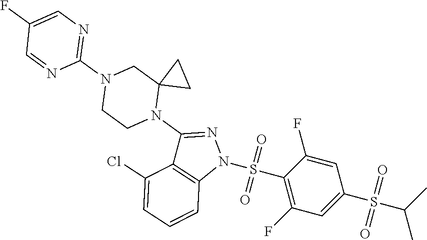 [SMILES: CC(C)S(=O)(=O)c1cc(F)c(S(=O)(=O)n2nc(N3CCN(c4ncc(F)cn4)CC34CC4)c3c(Cl)cccc32)c(F)c1]

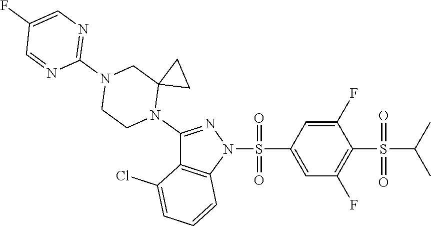 [SMILES: CC(C)S(=O)(=O)c1c(F)cc(S(=O)(=O)n2nc(N3CCN(c4ncc(F)cn4)CC34CC4)c3c(Cl)cccc32)cc1F]

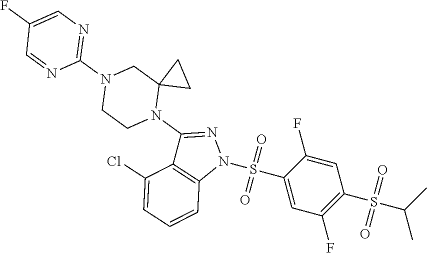 [SMILES: CC(C)S(=O)(=O)c1cc(F)c(S(=O)(=O)n2nc(N3CCN(c4ncc(F)cn4)CC34CC4)c3c(Cl)cccc32)cc1F]

In another aspect, the invention generally relates to a pharmaceutical composition comprising a compound disclosed herein.

In yet another aspect, the inventio generally relates to a pharmaceutical composition comprising a compound of structural formula I:

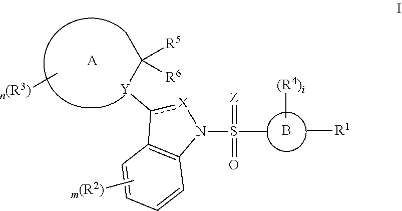

or a pharmaceutically acceptable form or an isotope derivative thereof, wherein
Ring A is a 4-, 5- or 6-membered substituted heterocycle;
Ring B a substituted or unsubstituted, 6-membered aryl or heteroaryl ring, 5-membered heteroaryl ring, or a substituted or unsubstituted bi- or multi-cyclic carbocyclic or heterocyclic ring;
X is CH or N;
Y is CH or N;
Z is O or NH;
R¹ is selected from the group consisting of C₁₋₅ alkyl, C₃₋₇ cycloalkyl, heterocyclic, halo, OR, SR, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, wherein the alkyl, cycloalkyl, heterocyclic, R and R′ is independently substituted with 0-6 (e.g., 0, 1, 2, 3, 4, 5 or 6) F's;
each R² is independently selected from the group consisting of C₁₋₆ alkyl, halo, oxo, OH, CN, OR, NRR′, N(R)C(═O) RR′, N(R)C(═O)(O)RR′, OC(═O)NRR′, C(═O)R, C(═O)NRR′, N(R)S(O)₂RR′, S(O)₂R, or S(O)₂NRR′;
each R³ is independently selected from the group consisting of C₁₋₅ alkyl, C₃₋₇ cycloalkyl, heterocycle, heteroaryl, O, halo, CF₃, CH₂CF₃, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, which where applicable is optionally substituted with 1, 2 or 3 groups selected from halo, CF₃, CH₂CF₃, CN, OH, C₁₋₅ alkyl, and C₃₋₇ cycloalkyl; two R³'s attached to the same carbon atom together form a C═O; or two R³'s, together with the carbon and/or nitrogen atom(s) to which they are attached, form a 3-, 4-, 5- or 6-membered substituted or unsubstituted carbocycle or heterocycle;
each R⁴ is independently selected from the group consisting of H, halo, CN, CF₃, C₁₋₅ alkyl, C₃₋₇ cycloalkyl and heterocyclic;
R⁵ and R⁶ are selected as follows:
   each of R⁵ and R⁶ is independently selected from C₁-C₃ alkyl, C₁-C₃ alkoxy, C₂-C₃ alkenyl, C₂-C₃ alkynyl, OH, CN, where applicable optionally substituted with 1-5 groups selected from C₁-C₃ alkyl, C₁-C₃ alkoxy, C₂-C₃ alkenyl, C₂-C₃ alkynyl, halo, OH and CN;
   one of R⁵ and R⁶ is CH₃, CH₂CH₃, OCH₃ or OCH₂CH₃ and the other is H;
   one of R⁵ and R⁶ is CF₃, CFHCH₃, CH₂CFH₂, CF₂CH₃, CFHCF₂H, CH₂CF₂H, CH₂CF₃, CFHCFH₂ or CF₂CF₃ and the other is H;
   one of R⁵ and R⁶ is CH═CH₂, CF═CH₂, CH═CFH, CH═CF₂, CF═CFH or CF═CF₂ and the other is H;
   one of R⁵ and R⁶ is C═CH, C═CF or CN and the other is H;
   one of R⁵ and R⁶ is a substituted or unsubstituted cyclopropyl, cyclobutyl or cyclopentyl ring and the other is H; or
   R⁵ and R⁶, together with the carbon atom to which they are attached to, are linked to form a substituted or unsubstituted 3- to 6-membered carbocyclic or heterocyclic ring;
each of R and R′ is independently H, or C₁₋₆ alkyl or cycloalkyl, optionally, R and R′, together with the nitrogen or carbon atom to which they are attached, form a 3- to 6-membered ring, each optionally substituted with 0-3 substituents independently selected from the group consisting of C₁₋₃ alkyl, halo, OH, OC₁₋₃ alkyl, and CN;
m is 0, 1, 2, 3 or 4;
n is 0, 1, 2, 3, 4 or 5; and
i is 0, 1 or 2,
   effective to treat, or reduce one or more diseases or disorders, in a mammal, including a human, and a pharmaceutically acceptable excipient, carrier, or diluent.

In yet another aspect, the invention generally relates to a unit dosage form comprising a pharmaceutical composition disclosed herein.

In certain embodiments, the unit dosage form is a tablet or a capsule.

In yet another aspect, the invention generally relates to a method for treating or reducing a disease or disorder, comprising administering to a subject in need thereof a pharmaceutical composition comprising a compound disclosed herein.

In certain embodiments, the disease or disorder is mediated by loss-of-function in TRPML1, including ML4 and NPC.

In certain embodiments, the disease or disorder is a lysosome storage disease, or a related disease or disorder.

In certain embodiments, the disease or disorder is selected from the group consisting of age-related neurodegenerative disease, including Alzheimer's Disease, Parkinson's Disease, and Huntington's Disease, or a related disease or disorder.

In certain embodiments, the disease or disorder is muscular dystrophy, or a related disease or disorder.

In certain embodiments, the disease or disorder is oxidative stress or ROS, or a related disease or disorder.

In yet another aspect, the invention generally relates to a method for treating or reducing the effect of aging comprising administering to a subject in need thereof a pharmaceutical composition comprising a compound disclosed herein.

In certain embodiments, the effect of aging comprises skin aging.

In certain embodiments, the effect of aging comprises photoaging.

In yet another aspect, the invention generally relates to a method for treating or reducing oxidative stress or ROS related diseases or disorder, comprising administering to a subject in need thereof an effective amount of a TRPML1 agonist or a composition comprising of a TRPML1 agonist.

In yet another aspect, the invention generally relates to a method for treating or reducing oxidative stress or ROS related diseases or disorder, comprising administering to a subject in need thereof a pharmaceutical composition comprising a compound disclosed herein.

In certain embodiments, administration is via oral administration.

In certain embodiments, administration is via topical administration.

In yet another aspect, the invention generally relates to use of a compound disclosed herein, and a pharmaceutically acceptable excipient, carrier, or diluent, in preparation of a medicament for treating a disease or disorder.

In certain embodiments, the disease or disorder is selected from the group consisting of age-related neurodegenerative disease, including Alzheimer's Disease, Parkinson's Disease, and Huntington's Disease, or a related disease or disorder.

In certain embodiments, the disease or disorder is muscular dystrophy, or a related disease or disorder.

In certain embodiments, the disease or disorder is oxidative stress or ROS, or a related disease or disorder.

In certain embodiments, the disease or disorder is skin aging or photoaging.

In certain embodiments, the compound is any of those shown in Table 1.

The specific approaches and compounds disclosed herein are not intended to be limiting. The chemical structures in the schemes herein depict variables that are hereby defined commensurately with chemical group definitions (moieties, atoms, etc.) of the corresponding position in the compound formulae herein, whether identified by the same variable name (e.g., R¹, R², R, R′, X, etc.) or not. The suitability of a chemical group in a compound structure for use in synthesis of another compound structure is within the knowledge of one of ordinary skill in the art. Additional methods of synthesizing compounds of the formulae herein and their synthetic precursors, including those within routes not explicitly shown in schemes herein, are within the means of chemists of ordinary skill in the art. Methods for optimizing reaction conditions, if necessary minimizing competing by-products, are known in the art. The methods described herein may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P.G.M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The methods delineated herein contemplate converting compounds of one formula to compounds of another formula. The process of converting refers to one or more chemical transformations, which can be performed in situ, or with isolation of intermediate compounds. The transformations can include reacting the starting compounds or intermediates with additional reagents using techniques and protocols known in the art, including those in the references cited herein. Intermediates can be used with or without purification (e.g., filtration, distillation, sublimation, crystallization, trituration, solid phase extraction, and chromatography).

Combinations of substituents and variables envisioned by this invention are only those that result in the formation of stable compounds.

Certain compounds of the present invention may exist in particular geometric or stereoisomeric forms. The present invention contemplates all such compounds, including cis- and trans-isomers, atropisomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Solvates and polymorphs of the compounds of the invention are also contemplated herein. Solvates of the compounds of the present invention include, for example, hydrates.

The invention also provides compositions comprising an effective amount of a compound of any of the formulae herein, or a pharmaceutically acceptable salt, solvate, hydrate, polymorph or prodrug, if applicable, of said compound; and an acceptable carrier. Preferably, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, PA (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary, shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein. Methods of formulating such slow or controlled release compositions of pharmaceutically active ingredients, such as those herein and other compounds known in the art, are known in the art and described in several issued US Patents, some of which include, but are not limited to, U.S. Pat. Nos. 4,369,172; and 4,842,866, and references cited therein. Coatings can be used for delivery of compounds to the intestine (see, e.g., U.S. Pat. Nos. 6,638,534, 5,217,720, and 6,569,457, 6,461,631, 6,528,080, 6,800,663, and references cited therein). A useful formulation for the compounds of this invention is the form of enteric pellets of which the enteric layer comprises hydroxypropylmethylcellulose acetate succinate.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or central nervous system) relative to the parent species. Preferred prodrugs include derivatives where a group that enhances aqueous solubility or active transport through the gut membrane is appended to the structure of formulae described herein. (See, e.g., Alexander, et al. 1988 J Med Chem 31, 318-322; Bundgaard 1985 Elsevier: Amsterdam 1-92; Bundgaard, et al. 1987 J Med Chem 30, 451-454; Bundgaard, H. A Textbook of Drug Design and Development; Harwood Academic Publ.: Switzerland, 1991, 113-191; Digenis, et al. Handbook of Experimental Pharmacology 1975 28, 86-112; Friis, et al. A Textbook of Drug Design and Development; 2 ed.; Overseas Publ.: Amsterdam, 1996, 351-385; Pitman 1981 Med Res Rev 1, 189-214.)

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

In another embodiment, a composition of the present invention further comprises a second therapeutic agent. The second therapeutic agent includes any compound or therapeutic agent known to have or that demonstrates advantageous properties when administered alone or with a compound of any of the formulae herein. Drugs that could be usefully combined with these compounds include other kinase inhibitors and/or other chemotherapeutic agents for the treatment of the diseases and disorders discussed above.

Such agents are described in detail in the art. Preferably, the second therapeutic agent is an agent useful in the treatment or prevention of cancer.

Even more preferably the second therapeutic agent co-formulated with a compound of this invention is an agent useful in the treatment of TRPML mediated disease/disorders. In another embodiment, the invention provides separate dosage forms of a compound of this invention and a second therapeutic agent that are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich, et al. 1966 Cancer Chemother Rep 50: 219. Body surface area may be approximately determined from height and weight of the patient. (See, e.g., Scientific Tables, Geigy Pharmaceuticals, Ardley, N.Y., 1970, 537.) An effective amount of a compound of this invention can range from about 0.001 mg/kg to about 500 mg/kg, more preferably 0.01 mg/kg to about 50 mg/kg, more preferably 0.1 mg/kg to about 2.5 mg/kg. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. (See, e.g., Wells, et al., eds. 2000 Pharmacotherapy Handbook, 2nd Edition, Appleton and Lange, Stamford, Conn.; PDR Pharmacopoeia, Tarascon Pocket Pharmacopoeia 2000, Deluxe Edition, Tarascon Publishing, Loma Linda, Calif 2000, each of which references are entirely incorporated herein by reference.

It is expected that some of the second therapeutic agents referenced above will act synergistically with the compounds of this invention. When this occurs, its will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

The invention also provides a method of treating a subject suffering from or susceptible to a disease or disorder or symptom thereof (e.g., those delineated herein) comprising the step of administering to said subject an effective amount of a compound or a composition of this invention. Some diseases are well known in the art and are also disclosed herein.

In certain embodiments, the methods disclosed herein are suitable for treating diseases or disorders that are mediated by the TRPMLs. In certain embodiments, the methods disclosed herein are suitable for treating disease or disorders that are mediated by loss-of-function in TRPML1, including ML4 and NPC.

In certain embodiments, the disease is one of the lysosomal storage diseases, such as Niemen-Pick C (NPC) disease.

In certain embodiments, the methods disclosed herein are suitable for treating diseases or disorders that are age-related including common neurodegenerative diseases, such as AD, PD, and HD.

In certain embodiments, the methods disclosed herein are suitable for treating type IV Mucolipidosis (ML4), a neurodegenerative LSD caused by human mutations in TRPML1.

In certain embodiments, the methods disclosed herein are suitable for treating certain metabolic diseases such as glycogen storage diseases (GSDs) and nonalcoholic fatty liver disease (NAFLD) including nonalcoholic steatohepatitis (NASH).

In certain embodiments, the methods disclosed herein are suitable for treating cancers in which TRPML1 is overexpressed in cancer cells,

In certain embodiments, the methods disclosed herein are suitable for treating a ROS or oxidative stress related disease or disorder.

In certain embodiments, the methods disclosed herein are suitable for treating diseases or disorders due or related to ageing.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention comprising both a compound of the invention and a second therapeutic agent to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif (2000), and other medical texts. However, it is well within the skilled artisan's purview to determine the second therapeutic agent's optimal effective-amount range.

In one embodiment of the invention where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of any of the formulae herein alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of the formulae herein for use in the treatment or prevention in a subject of a disease, disorder or symptom thereof delineated herein.

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabeling/assay techniques, blotting/chemiluminescence methods, real-time PCR, and the like.

The present invention also provides kits for use to treat diseases, disorders, or symptoms thereof, including those delineated herein. These kits comprise: a) a pharmaceutical composition comprising a compound of any of the formula herein or a salt thereof; or a prodrug, or a salt of a prodrug thereof; or a hydrate, solvate, or polymorph thereof, wherein said pharmaceutical composition is in a container; and b) instructions describing a method of using the pharmaceutical composition to treat the disease, disorder, or symptoms thereof, including those delineated herein.

The container may be any vessel or other sealed or sealable apparatus that can hold said pharmaceutical composition. Examples include bottles, divided or multi-chambered holders or bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. Preferably, the container is a blister pack.

The kit may additionally comprise information and/or instructions for the physician, pharmacist or subject. Such memory aids include numbers printed on each chamber or division containing a dosage that corresponds with the days of the regimen which the tablets or capsules so specified should be ingested, or days of the week printed on each chamber or division, or a card which contains the same type of information.

The following examples are meant to be illustrative of the practice of the invention and not limiting in any way.

### EXAMPLES

The structures depicted herein may contain certain —NH—, —NH₂ (amino) and —OH (hydroxyl) groups where the corresponding hydrogen atom(s) do not explicitly appear; however, they are to be read as —NH—, —NH₂ or —OH as the case may be. In certain structures, a stick bond is drawn and is meant to depict a methyl group.

TABLE 2
Results of TFEB Nuclear Translocation Assay

| Compound | EC₅₀ of TFEB | |
|---|---|---|
| number | activation (nM) | Name |
| 1 | +++ | 1-[(5-tert-butylsulfonyl-2-thienyl)sulfonyl]-4-chloro-3-[7-(5- |
| | | chloropyrimidin-2-yl)-4,7-diazaspiro[2.5]octan-4-yl]indazole |
| 2 | +++ | 4-chloro-3-[7-(5-chloropyrimidin-2-yl)-4,7- |
| | | diazaspiro[2.5]octan-4-yl]-1-(4- |
| | | isopropylsulfonylphenyl)sulfonyl-indazole |
| 3 | +++ | 4-chloro-6-fluoro-3-[7-(5-fluoropyrimidin-2-yl)-4,7- |
| | | diazaspiro[2.5]octan-4-yl]-1-(4- |
| | | isopropylsulfonylphenyl)sulfonyl-indazole |
| 4 | +++ | 4-chloro-3-[7-(5-chloropyrimidin-2-yl)-4,7- |
| | | diazaspiro[2.5]octan-4-yl]-1-(3- |
| | | isopropylsulfonylphenyl)sulfonyl-indazole |
| 5 | +++ | 4-chloro-3-[4-(5-chloropyrimidin-2-yl)-2-cyclopropyl-piperazin- |
| | | 1-yl]-1-[(5-isopropylsulfonyl-2-thienyl)sulfonyl]indazole |
| 6 | +++ | 4-chloro-1-[(5-isopropylsulfonyl-2-thienyl)sulfonyl]-3-[7-(2,2,2- |
| | | trifluoroethyl)-4,7-diazaspiro[2.5]octan-4-yl]indazole |
| 7 | +++ | 4-[4-chloro-1-[(5-isopropylsulfonyl-2-thienyl)sulfonyl]indazol- |
| | | 3-yl]-7-oxa-4-azaspiro[2.5]octane |
| 8 | +++ | 4-[4-chloro-1-[(5-isopropylsulfonyl-2-thienyl)sulfonyl]indazol- |
| | | 3-yl]-4-azaspiro[2.5]octan-7-one |
| 9 | +++ | 1-[4-[4-chloro-1-[(5-isopropylsulfonyl-2- |
| | | thienyl)sulfonyl]indazol-3-yl]-4,7-diazaspiro[2.5]octan-7-yl]- |
| | | 2,2,2-trifluoro-ethanone |
| 10 | +++ | 4-chloro-3-[7-(5-fluoropyrimidin-2-yl)-4,7-diazaspiro[2.5]octan- |
| | | 4-yl]-1-(4-isopropylsulfonylphenyl)sulfonyl-indazole |
| 11 | +++ | 4-chloro-3-(7-fluoro-4-azaspiro[2.5]octan-4-yl)-1-[(5- |
| | | isopropylsulfonyl-2-thienyl)sulfonyl]indazole |
| 12 | +++ | 4-chloro-3-(7,7-difluoro-4-azaspiro[2.5]octan-4-yl)-1-[(5- |
| | | isopropylsulfonyl-2-thienyl)sulfonyl]indazole |
| 13 | +++ | 4-(4-chloro-1-((5-(isopropylsulfonyl)thiophen-2-yl)sulfonyl)- |
| | | 1H-indazol-3-yl)-N,N-dimethyl-4-azaspiro[2.5]octan-7-amine |
| 14 | +++ | 4-chloro-1-((5-(isopropylsulfonyl)thiophen-2-yl)sulfonyl)-3-(7- |
| | | ((trifluoromethyl)sulfonyl)-4,7-diazaspiro[2.5]octan-4-yl)-1H- |
| | | indazole |
| 15 | +++ | 3-(7-(5-chloropyrimidin-2-yl)-4,7-diazaspiro[2.5]octan-4-yl)-4- |
| | | fluoro-1-((4-(isopropylsulfonyl)phenyl)sulfonyl)-1H-indazole |
| 16 | +++ | 4-fluoro-3-(7-(5-fluoropyrimidin-2-yl)-4,7-diazaspiro[2.5]octan- |
| | | 4-yl)-1-((4-(isopropylsulfonyl)phenyl)sulfonyl)-1H-indazole |
| 17 | +++ | 4-chloro-1-((5-(isopropylsulfonyl)thiophen-2-yl)sulfonyl)-3-(4- |
| | | azaspiro[2.4]heptan-4-yl)-1H-indazole |
| 18 | +++ | 4-chloro-3-(4-(5-fluoropyrimidin-2-yl)-2,2-dimethylpiperazin-1- |
| | | yl)-1-((4-(isopropylsulfonyl)phenyl)sulfonyl)-1H-indazole |
| 19 | +++ | 4,6-difluoro-3-(7-(5-fluoropyridin-2-yl)-4,7- |
| | | diazaspiro[2.5]octan-4-yl)-1-((4- |
| | | (isopropylsulfonyl)phenyl)sulfonyl)-1H-indazole |
| 20 | +++ | 4,6-difluoro-3-(7-(5-fluoropyrimidin-2-yl)-4,7- |
| | | diazaspiro[2.5]octan-4-yl)-1-((4- |
| | | (isopropylsulfonyl)phenyl)sulfonyl)-1H-indazole |
| 21 | +++ | 4-chloro-6-fluoro-3-(7-(5-fluoropyrimidin-2-yl)-4,7- |
| | | diazaspiro[2.5]octan-4-yl)-1-((4- |
| | | (isopropylsulfonyl)phenyl)sulfonyl)-1H-indazole |
| 22 | +++ | 4-chloro-1-((4-((2-fluoropropan-2-yl)sulfonyl)phenyl)sulfonyl)- |
| | | 3-(7-(5-fluoropyrimidin-2-yl)-4,7-diazaspiro[2.5]octan-4-yl)- |
| | | 1H-indazole |
| 23 | +++ | 4-chloro-3-(7-(5-fluoropyridin-2-yl)-4,7-diazaspiro[2.5]octan-4- |
| | | yl)-1-((4-(isopropylsulfonyl)phenyl)sulfonyl)-1H-indazole |
| 24 | +++ | 4-chloro-3-(7-(3,5-difluoropyridin-2-yl)-4,7- |
| | | diazaspiro[2.5]octan-4-yl)-1-((4- |
| | | (isopropylsulfonyl)phenyl)sulfonyl)-1H-indazole |
| 25 | +++ | 4-fluoro-3-(7-(5-fluoropyridin-2-yl)-4,7-diazaspiro[2.5]octan-4- |
| | | yl)-1-((4-(isopropylsulfonyl)phenyl)sulfonyl)-1H-indazole |
| 26 | +++ | 3-(7-(3,5-difluoropyridin-2-yl)-4,7-diazaspiro[2.5]octan-4-yl)-4- |
| | | fluoro-1-((4-(isopropylsulfonyl)phenyl)sulfonyl)-1H-indazole |
| 27 | +++ | 4-chloro-3-(7-(3,5-difluoropyridin-2-yl)-4,7- |
| | | diazaspiro[2.5]octan-4-yl)-6-fluoro-1-((4- |
| | | (isopropylsulfonyl)phenyl)sulfonyl)-1H-indazole |
| 28 | +++ | 3-(7-(3,5-difluoropyridin-2-yl)-4,7-diazaspiro[2.5]octan-4-yl)- |
| | | 4,6-difluoro-1-((4-(isopropylsulfonyl)phenyl)sulfonyl)-1H- |
| | | indazole |
| 29 | +++ | 4-chloro-1-((4-(cyclopropylsulfonyl)phenyl)sulfonyl)-3-(7-(5- |
| | | fluoropyrimidin-2-yl)-4,7-diazaspiro[2.5]octan-4-yl)-1H- |
| | | indazole |
| 30 | +++ | 4-chloro-3-(7-(5-chloro-3-fluoropyridin-2-yl)-4,7- |
| | | diazaspiro[2.5]octan-4-yl)-1-((5-(isopropylsulfonyl)thiophen-2- |
| | | yl)sulfonyl)-1H-indazole |
| 31 | +++ | 4-chloro-3-(7-(3,5-difluoropyridin-2-yl)-4,7- |
| | | diazaspiro[2.5]octan-4-yl)-7-fluoro-1-((5- |
| | | (isopropylsulfonyl)thiophen-2-yl)sulfonyl)-1H-indazole |
| 32 | +++ | 3-(7-(3,5-difluoropyridin-2-yl)-4,7-diazaspiro[2.5]octan-4-yl)- |
| | | 4,7-difluoro-1-((5-(isopropylsulfonyl)thiophen-2-yl)sulfonyl)- |
| | | 1H-indazole |
| 33 | +++ | 3-(7-(5-chloro-3-fluoropyridin-2-yl)-4,7-diazaspiro[2.5]octan-4- |
| | | yl)-4,7-difluoro-1-((5-(isopropylsulfonyl)thiophen-2- |
| | | yl)sulfonyl)-1H-indazole |
| 34 | +++ | 4,7-difluoro-3-(7-(5-fluoropyrimidin-2-yl)-4,7- |
| | | diazaspiro[2.5]octan-4-yl)-1-((5-(isopropylsulfonyl)thiophen-2- |
| | | yl)sulfonyl)-1H-indazole |
| 35 | +++ | 4-chloro-3-(7-(5-chloro-3-fluoropyridin-2-yl)-4,7- |
| | | diazaspiro[2.5]octan-4-yl)-6-fluoro-1-((5- |
| | | (isopropylsulfonyl)thiophen-2-yl)sulfonyl)-1H-indazole |
| 36 | +++ | 4-chloro-3-(7-(5-chloropyrimidin-2-yl)-4,7- |
| | | diazaspiro[2.5]octan-4-yl)-6-fluoro-1-((5- |
| | | (isopropylsulfonyl)thiophen-2-yl)sulfonyl)-1H-indazole |
| 37 | +++ | 4-chloro-3-(7-(3,5-difluoropyridin-2-yl)-4,7- |
| | | diazaspiro[2.5]octan-4-yl)-6-fluoro-1-((5- |
| | | (isopropylsulfonyl)thiophen-2-yl)sulfonyl)-1H-indazole |
| 38 | +++ | 4-chloro-6-fluoro-3-(7-(5-fluoropyrimidin-2-yl)-4,7- |
| | | diazaspiro[2.5]octan-4-yl)-1-((5-(isopropylsulfonyl)thiophen-2- |
| | | yl)sulfonyl)-1H-indazole |
| 39 | +++ | 5-chloro-2-(4-(4-chloro-6-fluoro-1-((5- |
| | | (isopropylsulfonyl)thiophen-2-yl)sulfonyl)-1H-indazol-3-yl)- |
| | | 4,7-diazaspiro[2.5]octan-7-yl)thiazole |
| 40 | +++ | 3-(7-(3,5-difluoropyridin-2-yl)-4,7-diazaspiro[2.5]octan-4-yl)- |
| | | 4,6-difluoro-1-((5-(isopropylsulfonyl)thiophen-2-yl)sulfonyl)- |
| | | 1H-indazole |
| 41 | +++ | 4-chloro-6-fluoro-3-(7-(5-fluoropyridin-2-yl)-4,7- |
| | | diazaspiro[2.5]octan-4-yl)-1-((5-(isopropylsulfonyl)thiophen-2- |
| | | yl)sulfonyl)-1H-indazole |
| 42 | +++ | 4-chloro-3-(7-(5-fluoropyridin-2-yl)-4,7-diazaspiro [2.5] octan- |
| | | 4-yl)-1 |
| | | -((5-(isopropylsulfonyl) thiophen-2-yl) sulfonyl)-1H-indazole |
| 43 | +++ | 4-chloro-3-[7-(5-chloropyrimidin-2-yl)-4,7-diazaspiro [2.5] |
| | | octan-4-yl] |
| | | -1-[(5-isopropylsulfonyl-2-thienyl) sulfonyl] indazole |
| 44 | +++ | 4-chloro-3-[7-(5-fluoropyrimidin-2-yl)-4,7-diazaspiro[2.5]octan- |
| | | 4-yl]- |
| | | 1-[(5-isopropylsulfonyl-2-thienyl)sulfonyl]indazole |
| 45 | +++ | 4-chloro-3-[7-(3,5-difluoro-2-pyridyl)-4,7-diazaspiro [2.5] |
| | | octan-4-yl] |
| | | -1-[(5-isopropylsulfonyl-2-thienyl) sulfonyl] indazole |
| 46 | +++ | 5-chloro-2-[4-[4-chloro-1-[(5-isopropylsulfonyl-2-thienyl) |
| | | sulfonyl] indazol |
| | | -3-yl]-4,7-diazaspiro [2.5] octan-7-yl]thiazole |
| 47 | +++ | 4-chloro-3-[7-(5-chloro-2-pyridyl)-4,7-diazaspiro[2.5]octan-4- |
| | | yl]-1- |
| | | [(5-isopropylsulfonyl-2-thienyl)sulfonyl]indazole |
| 48 | +++ | 1-((5-(tert-butylsulfonyl)thiophen-2-yl)sulfonyl)-4-chloro-3-(7- |
| | | (5-fluoropy |
| | | ridin-2-yl)-4,7-diazaspiro[2.5]octan-4-yl)-1H-indazole |
| 49 | +++ | 1-((5-(tert-butylsulfonyl)thiophen-2-yl)sulfonyl)-4-chloro-3-(7- |
| | | (5-fluoropy |
| | | rimidin-2-yl)-4,7-diazaspiro[2.5]octan-4-yl)-1H-indazole |
| 50 | +++ | 4-chloro-3-(2-cyclopropyl-4-(5-fluoropyrimidin-2-yl)piperazin- |
| | | 1-yl)-1-((4-(isopropylsulfonyl)phenyl)sulfonyl)-1H-indazole |
| 51 | +++ | 4-chloro-3-(2-cyclopropyl-4-(5-fluoropyrimidin-2-yl)piperazin- |
| | | 1-yl)-6-fluoro-1-((4-(isopropylsulfonyl)phenyl)sulfonyl)-1H- |
| | | indazole |
| 52 | +++ | 4-chloro-3-(7-(5-fluoropyrimidin-2-yl)-4,7-diazaspiro[2.5]octan- |
| | | 4-yl)-1-((4-((trifluoromethyl)sulfonyl)phenyl)sulfonyl)-1H- |
| | | indazole |
| 53 | +++ | 4-chloro-3-(7,7-difluoro-4-azaspiro[2.5]octan-4-yl)-1-((4- |
| | | (isopropylsulfonyl)phenyl)sulfonyl)-1H-indazole |
| 54 | +++ | 4-chloro-3-(7,7-dichloro-4-azaspiro[2.5]octan-4-yl)-1-((4- |
| | | (isopropylsulfonyl)phenyl)sulfonyl)-1H-indazole |
| 55 | +++ | 1-[4-[4-chloro-1-(4-isopropylsulfonylphenyl) sulfonyl-indazol- |
| | | 3-yl]-4,7-diazaspiro [2.5] octan-7-yl]-2,2,2-trifluoro-ethanone |
| 56 | +++ | 1-(4-(4-chloro-1-((4-(isopropylsulfonyl)phenyl)sulfonyl)-1H- |
| | | indazol-3-yl)-4,7-diazaspiro[2.5]octan-7-yl)ethanone |
| 57 | +++ | (4-(4-chloro-1-((4-(isopropylsulfonyl)phenyl)sulfonyl)-1H- |
| | | indazol-3-yl)-4,7-diazaspiro[2.5]octan-7- |
| | | yl)(cyclopropyl)methanone |
| 58 | +++ | 4-chloro-3-(7-(5-fluoropyrimidin-2-yl)-4,7-diazaspiro[2.5]octan- |
| | | 4-yl)-1-((4-(propan-2-ylsulfonimidoyl)phenyl)sulfonyl)-1H- |
| | | indazole |
| 59 | +++ | 4-chloro-6-fluoro-1-((1-(isopropylsulfonyl)-1H-pyrrol-3- |
| | | yl)sulfonyl)-3-(7-(2,2,2-trifluoroethyl)-4,7-diazaspiro[2.5]octan- |
| | | 4-yl)-1H-indazole |
| 60 | +++ | (S)-4-chloro-3-(2-cyclopropyl-4-(5-fluoropyrimidin-2- |
| | | yl)piperazin-1-yl)-1-((4-(isopropylsulfonyl)phenyl)sulfonyl)- |
| | | 1H-indazole |
| 61 | +++ | (R)-4-chloro-3-(2-cyclopropyl-4-(5-fluoropyrimidin-2- |
| | | yl)piperazin-1-yl)-1-((4-(isopropylsulfonyl)phenyl)sulfonyl)- |
| | | 1H-indazole |
| 62 | +++ | 4-chloro-1-((3-fluoro-4-(isopropylsulfonyl)phenyl)sulfonyl)-3- |
| | | (7-(5-fluoropyrimidin-2-yl)-4,7-diazaspiro[2.5]octan-4-yl)-1H- |
| | | indazole |
| 63 | +++ | 4-chloro-3-(2-cyclopropylpiperidin-1-yl)-1-((4- |
| | | (isopropylsulfonyl)phenyl)sulfonyl)-1H-indazole |
| 64 | +++ | 4-chloro-3-[7-(5-fluoropyrimidin-2-yl)-4,7-diazaspiro[2.5]octan- |
| | | 4-yl]-1-(2,3,5,6-tetradeuterio-4-isopropylsulfonyl- |
| | | phenyl)sulfonyl-indazole |
| 65 | +++ | 4-chloro-1-((4-(isopropylsulfonyl)phenyl)sulfonyl)-3-(7- |
| | | (pyrimidin-2-yl)-4,7-diazaspiro[2.5]octan-4-yl)-1H-indazole |
| 66 | +++ | 4-chloro-1-((4-(isopropylsulfonyl)phenyl)sulfonyl)-3-(2- |
| | | (trifluoromethyl)piperidin-1-yl)-1H-indazole |
| 67 | +++ | 4-chloro-3-(4-(5-fluoropyrimidin-2-yl)-2- |
| | | (trifluoromethyl)piperazin-1-yl)-1-((4- |
| | | (isopropylsulfonyl)phenyl)sulfonyl)-1H-indazole |
| 68 | +++ | (R)-4-chloro-3-(4-(5-fluoropyrimidin-2-yl)-2- |
| | | (trifluoromethyl)piperazin-1-yl)-1-((4- |
| | | (isopropylsulfonyl)phenyl)sulfonyl)-1H-indazole |
| 69 | +++ | (S)-4-chloro-3-(4-(5-fluoropyrimidin-2-yl)-2- |
| | | (trifluoromethyl)piperazin-1-yl)-1-((4- |
| | | (isopropylsulfonyl)phenyl)sulfonyl)-1H-indazole |
| 70 | +++ | (R)-1-(4-(4-chloro-1-((4-(isopropylsulfonyl)phenyl)sulfonyl)- |
| | | 1H-indazol-3-yl)-3-(trifluoromethyl)piperazin-1-yl)-2,2,2- |
| | | trifluoroethanone |
| 71 | +++ | (S)-1-(4-(4-chloro-1-((4-(isopropylsulfonyl)phenyl)sulfonyl)- |
| | | 1H-indazol-3-yl)-3-(trifluoromethyl)piperazin-1-yl)-2,2,2- |
| | | trifluoroethanone |
| 72 | +++ | (R)-4-fluoro-3-(4-(5-fluoropyrimidin-2-yl)-2- |
| | | (trifluoromethyl)piperazin-1-yl)-1-((4- |
| | | (isopropylsulfonyl)phenyl)sulfonyl)-1H-indazole |
| 73 | +++ | (S)-4-fluoro-3-(4-(5-fluoropyrimidin-2-yl)-2- |
| | | (trifluoromethyl)piperazin-1-yl)-1-((4- |
| | | (isopropylsulfonyl)phenyl)sulfonyl)-1H-indazole |
| 74 | +++ | 1-[4-[4-chloro-1-(4-isopropylsulfonylphenyl) sulfonyl-indazol- |
| | | 3-yl]-3-(trifluoromethyl)piperazin-1-yl]-2,2-difluoro-ethanone |
| 75 | +++ | 4-chloro-1-((4-(isopropylsulfonyl)phenyl)sulfonyl)-3-(4-(2,2,2- |
| | | trifluoroethyl)-2-(trifluoromethyl)piperazin-1-yl)-1H-indazole |
| 76 | +++ | 1-(4-(4-chloro-1-((4-(difluoromethyl)phenyl)sulfonyl)-1H- |
| | | indazol-3-yl)-3-(trifluoromethyl)piperazin-1-yl)-2,2,2- |
| | | trifluoroethanone |
| 77 | +++ | 4-chloro-1-((4-(difluoromethyl)phenyl)sulfonyl)-3-(4-(5- |
| | | fluoropyrimidin-2-yl)-2-(trifluoromethyl)piperazin-1-yl)-1H- |
| | | indazole |
| 78 | +++ | 4-chloro-1-((4-(difluoromethyl)phenyl)sulfonyl)-3-(7-(5- |
| | | fluoropyrimidin-2-yl)-4,7-diazaspiro[2.5]octan-4-yl)-1H- |
| | | indazole |
| 79 | +++ | 4-chloro-3-(4-chloro-2-(trifluoromethyl)-5,6-dihydropyridin- |
| | | 1(2H)-yl)-1-((4-(isopropylsulfonyl)phenyl)sulfonyl)-1H- |
| | | indazole |
| 80 | +++ | (R)-4-chloro-3-(4-chloro-2-(trifluoromethyl)-5,6- |
| | | dihydropyridin-1(2H)-yl)-1-((4- |
| | | (isopropylsulfonyl)phenyl)sulfonyl)-1H-indazole |
| 81 | +++ | (S)-4-chloro-3-(4-chloro-2-(trifluoromethyl)-5,6-dihydropyridin- |
| | | 1(2H)-yl)-1-((4-(isopropylsulfonyl)phenyl)sulfonyl)-1H- |
| | | indazole |
| 82 | +++ | (R)-4-chloro-3-(4-chloro-6-(trifluoromethyl)-5,6- |
| | | dihydropyridin-1(2H)-yl)-1-((4- |
| | | (isopropylsulfonyl)phenyl)sulfonyl)-1H-indazole |
| 83 | +++ | (S)-4-chloro-3-(4-chloro-6-(trifluoromethyl)-5,6-dihydropyridin- |
| | | 1(2H)-yl)-1-((4-(isopropylsulfonyl)phenyl)sulfonyl)-1H- |
| | | indazole |
| 84 | +++ | 1-(4-chloro-1-((4-(isopropylsulfonyl)phenyl)sulfonyl)-1H- |
| | | indazol-3-yl)-2-(trifluoromethyl)piperidin-4-one |
| 85 | +++ | 4-chloro-3-[4,4-difluoro-2-(trifluoromethyl)-1-piperidyl]-1-(4- |
| | | isopropylsulfonylphenyl) sulfonyl-indazole |
| 86 | +++ | 4-chloro-3-(4-(difluoromethoxy)-2-(trifluoromethyl)piperidin-1- |
| | | yl)-1-((4-(isopropylsulfonyl)phenyl)sulfonyl)-1H-indazole |
| 87 | +++ | 4-chloro-3-(7-(difluoromethoxy)-4-azaspiro[2.5]octan-4-yl)-1- |
| | | ((4-(isopropylsulfonyl)phenyl)sulfonyl)-1H-indazole |
| 88 | + | 11-[4-chloro-1-[4-(1,1-difluoroethyl)phenyl]sulfonyl-indazol-3- |
| | | yl]-8-oxa-11-azadispiro[2.1.35.33]undecane |
| 89 | +++ | 4-chloro-1-((4-(3-fluorooxetan-3-yl)phenyl)sulfonyl)-3-(7-(5- |
| | | fluoropyrimidin-2-yl)-4,7-diazaspiro[2.5]octan-4-yl)-1H- |
| | | indazole |
| 90 | +++ | 4-chloro-1-((4-(1,1-difluoro-2-methylpropyl)phenyl)sulfonyl)-3- |
| | | (7-(5-fluoropyrimidin-2-yl)-4,7-diazaspiro[2.5]octan-4-yl)-1H- |
| | | indazole |
| 91 | +++ | 4-chloro-1-((4-(1,1-difluoroethyl)phenyl)sulfonyl)-6-fluoro-3- |
| | | (7-(5-fluoropyrimidin-2-yl)-4,7-diazaspiro[2.5]octan-4-yl)-1H- |
| | | indazole |
| 92 | +++ | 4-chloro-1-((4-(cyclopropyldifluoromethyl)phenyl)sulfonyl)-3- |
| | | (7-(5-fluoropyrimidin-2-yl)-4,7-diazaspiro[2.5]octan-4-yl)-1H- |
| | | indazole |
| 93 | +++ | 4-chloro-1-((4-((difluoromethyl)thio)phenyl)sulfonyl)-3-(7-(5- |
| | | fluoropyrimidin-2-yl)-4,7-diazaspiro[2.5]octan-4-yl)-1H- |
| | | indazole |
| 94 | +++ | 1-((4-(tert-butyl)phenyl)sulfonyl)-4-chloro-3-(7-(5- |
| | | fluoropyrimidin-2-yl)-4,7-diazaspiro[2.5]octan-4-yl)-1H- |
| | | indazole |
| 95 | +++ | 4-chloro-3-(7-(5-fluoropyrimidin-2-yl)-4,7-diazaspiro[2.5]octan- |
| | | 4-yl)-1-((4-(1,1,2,2-tetrafluoroethyl)phenyl)sulfonyl)-1H- |
| | | indazole |
| 96 | +++ | 4-chloro-1-((4-(1-fluoro-2-methylpropan-2-yl)phenyl)sulfonyl)- |
| | | 3-(7-(5-fluoropyrimidin-2-yl)-4,7-diazaspiro[2.5]octan-4-yl)- |
| | | 1H-indazole |
| 97 | +++ | 4-chloro-1-((4-(2-fluoropropan-2-yl)phenyl)sulfonyl)-3-(7-(5- |
| | | fluoropyrimidin-2-yl)-4,7-diazaspiro[2.5]octan-4-yl)-1H- |
| | | indazole |
| 98 | +++ | 4-chloro-3-(7-(5-fluoropyrimidin-2-yl)-4,7-diazaspiro[2.5]octan- |
| | | 4-yl)-1-((4-(1,1,2-trifluoroethyl)phenyl)sulfonyl)-1H-indazole |
| 99 | +++ | 4-chloro-1-((4-(1,1-difluoro-2-methylpropan-2- |
| | | yl)phenyl)sulfonyl)-3-(7-(5-fluoropyrimidin-2-yl)-4,7- |
| | | diazaspiro[2.5]octan-4-yl)-1H-indazole |
| 100 | +++ | 1-((2-(tert-butyl)pyrimidin-5-yl)sulfonyl)-4-chloro-3-(7-(5- |
| | | fluoropyrimidin-2-yl)-4,7-diazaspiro[2.5]octan-4-yl)-1H- |
| | | indazole |
| 101 | +++ | 4-chloro-3-(7-(5-fluoropyrimidin-2-yl)-4,7-diazaspiro[2.5]octan- |
| | | 4-yl)-1-((4-(1-(trifluoromethyl)cyclopropyl)phenyl)sulfonyl)- |
| | | 1H-indazole |
| 102 | +++ | 4-chloro-1-((4-(1,1-difluoropropyl)phenyl)sulfonyl)-3-(7-(5- |
| | | fluoropyrimidin-2-yl)-4,7-diazaspiro[2.5]octan-4-yl)-1H- |
| | | indazole |
| 103 | +++ | 4-chloro-1-((4-(1,1-difluoroethyl)phenyl)sulfonyl)-7-fluoro-3- |
| | | (7-(5-fluoropyrimidin-2-yl)-4,7-diazaspiro[2.5]octan-4-yl)-1H- |
| | | indazole |
| 104 | +++ | 4-chloro-1-((4-(1,1-difluoroethyl)phenyl)sulfonyl)-3-(2- |
| | | (trifluoromethyl)azetidin-1-yl)-1H-indazole |
| 105 | +++ | 4-chloro-1-((4-(1,1-difluoroethyl)phenyl)sulfonyl)-3-(2-(2- |
| | | fluoropropan-2-yl)-4-(5-fluoropyrimidin-2-yl)piperazin-1-yl)- |
| | | 1H-indazole |
| 106 | +++ | (S)-4-chloro-1-((4-(1,1-difluoroethyl)phenyl)sulfonyl)-3-(4-(5- |
| | | fluoropyrimidin-2-yl)-2-(trifluoromethyl)piperazin-1-yl)-1H- |
| | | indazole |
| 107 | +++ | (R)-4-chloro-1-((4-(1,1-difluoroethyl)phenyl)sulfonyl)-3-(4-(5- |
| | | fluoropyrimidin-2-yl)-2-(trifluoromethyl)piperazin-1-yl)-1H- |
| | | indazole |
| 108 | +++ | 3-(2-(tert-butyl)-4-(5-fluoropyrimidin-2-yl)piperazin-1-yl)-4- |
| | | chloro-1-((4-(1,1-difluoroethyl)phenyl)sulfonyl)-1H-indazole |
| 109 | +++ | (R)-4-chloro-1-((4-(isopropylsulfonyl)phenyl)sulfonyl)-3-(2- |
| | | (trifluoromethyl)pyrrolidin-1-yl)-1H-indazole |
| 110 | +++ | (S)-4-chloro-1-((4-(isopropylsulfonyl)phenyl)sulfonyl)-3-(2- |
| | | (trifluoromethyl)pyrrolidin-1-yl)-1H-indazole |
| 111 | +++ | 4-chloro-3-(7-(5-fluoropyrimidin-2-yl)-4,7-diazaspiro[2.5]octan- |
| | | 4-yl)-1-((1-(isopropylsulfonyl)-1H-pyrrol-3-yl)sulfonyl)-1H- |
| | | indazole |
| 112 | +++ | 4-chloro-3-(2-cyclopropyl-4-(5-fluoropyrimidin-2-yl)piperazin- |
| | | 1-yl)-1-((4-(difluoromethyl)phenyl)sulfonyl)-1H-indazole |
| 113 | +++ | 4-chloro-3-(7-(5-fluoropyrimidin-2-yl)-4,7-diazaspiro[2.5]octan- |
| | | 4-yl)-1-((4-(trifluoromethyl)phenyl)sulfonyl)-1H-indazole |
| 114 | +++ | 4-chloro-1-((4-((difluoromethyl)sulfonyl)phenyl)sulfonyl)-3-(7- |
| | | (5-fluoropyrimidin-2-yl)-4,7-diazaspiro[2.5]octan-4-yl)-1H- |
| | | indazole |
| 115 | +++ | 4-chloro-1-((4-(difluoromethoxy)phenyl)sulfonyl)-3-(7-(5- |
| | | fluoropyrimidin-2-yl)-4,7-diazaspiro[2.5]octan-4-yl)-1H- |
| | | indazole |
| 116 | +++ | 4-chloro-1-((4-(1,1-difluoroethyl)phenyl)sulfonyl)-3-(7-(5- |
| | | fluoropyrimidin-2-yl)-4,7-diazaspiro[2.5]octan-4-yl)-1H- |
| | | indazole |
| 117 | +++ | 4-chloro-3-(7-(5-fluoropyrimidin-2-yl)-4,7-diazaspiro[2.5]octan- |
| | | 4-yl)-1-((4-(trifluoromethoxy)phenyl)sulfonyl)-1H-indazole |
| 118 | +++ | 4-chloro-3-(7-(5-fluoropyrimidin-2-yl)-4,7-diazaspiro[2.5]octan- |
| | | 4-yl)-1-((4-((trifluoromethyl)thio)phenyl)sulfonyl)-1H-indazole |
| 119 | +++ | 4-chloro-1-((4-(1,1-difluoroethyl)phenyl)sulfonyl)-3-(7-(oxetan- |
| | | 3-yl)-4,7-diazaspiro[2.5]octan-4-yl)-1H-indazole |
| 120 | +++ | 4-chloro-1-((4-(1,1-difluoroethyl)phenyl)sulfonyl)-3-(4-(oxetan- |
| | | 3-yl)-2-(trifluoromethyl)piperazin-1-yl)-1H-indazole |
| 121 | +++ | (S)-4-chloro-1-((4-(1,1-difluoroethyl)phenyl)sulfonyl)-3-(2-(2- |
| | | fluoropropan-2-yl)-4-(5-fluoropyrimidin-2-yl)piperazin-1-yl)- |
| | | 1H-indazole |
| 122 | +++ | (R)-4-chloro-1-((4-(1,1-difluoroethyl)phenyl)sulfonyl)-3-(2-(2- |
| | | fluoropropan-2-yl)-4-(5-fluoropyrimidin-2-yl)piperazin-1-yl)- |
| | | 1H-indazole |
| 123 | +++ | 4-chloro-1-((4-(1,1-difluoroethyl)phenyl)sulfonyl)-3-(7-(3,5- |
| | | difluoropyridin-2-yl)-4,7-diazaspiro[2.5]octan-4-yl)-1H- |
| | | indazole |
| 124 | +++ | 4-chloro-3-(7-(4-chloro-2-fluorophenyl)-4,7- |
| | | diazaspiro[2.5]octan-4-yl)-1-((4-(1,1- |
| | | difluoroethyl)phenyl)sulfonyl)-1H-indazole |
| 125 | +++ | 4-chloro-3-(7-(2-chloro-4-fluorophenyl)-4,7- |
| | | diazaspiro[2.5]octan-4-yl)-1-((4-(1,1- |
| | | difluoroethyl)phenyl)sulfonyl)-1H-indazole |
| 126 | +++ | 4-chloro-1-((4-(1,1-difluoroethyl)phenyl)sulfonyl)-3-(7-(2,4- |
| | | difluorophenyl)-4,7-diazaspiro[2.5]octan-4-yl)-1H-indazole |
| 127 | +++ | 4-(4-chloro-1-((4-(1,1-difluoroethyl)phenyl)sulfonyl)-1H- |
| | | indazol-3-yl)-4,7-diazaspiro[2.5]octane-7-carbonyl fluoride |
| 128 | +++ | 4-chloro-1-((4-(1,1-difluoroethyl)phenyl)sulfonyl)-3-(7-(1- |
| | | fluoro-2-methylpropan-2-yl)-4,7-diazaspiro[2.5]octan-4-yl)-1H- |
| | | indazole |
| 129 | +++ | 4-chloro-1-((3-(1,1-difluoroethyl)bicyclo[1.1.1]pentan-1- |
| | | yl)sulfonyl)-3-(7-(5-fluoropyrimidin-2-yl)-4,7- |
| | | diazaspiro[2.5]octan-4-yl)-1H-indazole |
| 130 | +++ | 4-chloro-3-(7-(1,3-difluoro-2-methylpropan-2-yl)-4,7- |
| | | diazaspiro[2.5]octan-4-yl)-1-((4-(1,1- |
| | | difluoroethyl)phenyl)sulfonyl)-1H-indazole |
| 131 | + | 4-chloro-3-(7-(5-fluoropyrimidin-2-yl)-4,7-diazaspiro[2.5]octan- |
| | | 4-yl)-1-((3-(trifluoromethyl)bicyclo[1.1.1]pentan-1-yl)sulfonyl)- |
| | | 1H-indazole |
| 132 | + | 4-chloro-1-((3-fluorobicyclo[1.1.1]pentan-1-yl)sulfonyl)-3-(7- |
| | | (5-fluoropyrimidin-2-yl)-4,7-diazaspiro[2.5]octan-4-yl)-1H- |
| | | indazole |
| 133 | +++ | 1-((4-(1,1-difluoroethyl)phenyl)sulfonyl)-4-fluoro-3-(7-(5- |
| | | fluoropyrimidin-2-yl)-4,7-diazaspiro[2.5]octan-4-yl)-1H- |
| | | indazole |
| 134 | + | 1-((4-(1,1-difluoroethyl)phenyl)sulfonyl)-3-(7-(5- |
| | | fluoropyrimidin-2-yl)-4,7-diazaspiro[2.5]octan-4-yl)-1H- |
| | | indazol-4-ol |
| 135 | +++ | 4-chloro-1-((6-(1,1-difluoroethyl)pyridin-3-yl)sulfonyl)-3-(7-(5- |
| | | fluoropyrimidin-2-yl)-4,7-diazaspiro[2.5]octan-4-yl)-1H- |
| | | indazole |
| 136 | + | 4-chloro-1-(5-(1,1-difluoroethyl)pyrimidin-2-yl)-3-(7-(5- |
| | | fluoropyrimidin-2-yl)-4,7-diazaspiro[2.5]octan-4-yl)-1H- |
| | | indazole |
| 137 | +++ | 4-chloro-1-((2-(1,1-difluoroethyl)pyrimidin-5-yl)sulfonyl)-3-(7- |
| | | (5-fluoropyrimidin-2-yl)-4,7-diazaspiro[2.5]octan-4-yl)-1H- |
| | | indazole |
| 138 | +++ | 4-[4-chloro-1-(1-isopropylsulfonylpyrrol-3-yl) sulfonyl-indazol- |
| | | 3-yl]-3,5-dimethyl-morpholine |
| 139 | +++ | 3-(5-azaspiro [2.5] octan-5-yl)-4-chloro-1-(1- |
| | | isopropylsulfonylpyrrol-3-yl) sulfonyl-indazole |
| 140 | +++ | 4-chloro-3-[7-(5-chloro-3-fluoro-2-pyridyl)-4,7-diazaspiro [2.5] |
| | | octan-4-yl]-1-(1-isopropylsulfonylpyrrol-3-yl) sulfonyl-indazole |
| 141 | +++ | 4-chloro-3-(7-(3,5-difluoropyridin-2-yl)-4,7- |
| | | diazaspiro[2.5]octan-4-yl)-1-((1-(isopropylsulfonyl)-1H-pyrrol- |
| | | 3-yl)sulfonyl)-1H-indazole |
| 142 | +++ | 4-chloro-3-(7-(3,5-difluoropyridin-2-yl)-4,7- |
| | | diazaspiro[2.5]octan-4-yl)-7-fluoro-1-((1-(isopropylsulfonyl)- |
| | | 1H-pyrrol-3-yl)sulfonyl)-1H-indazole |
| 143 | +++ | 4-chloro-1-((1-(cyclopropylsulfonyl)-1H-pyrrol-3-yl)sulfonyl)- |
| | | 3-(7-(3,5-difluoropyridin-2-yl)-4,7-diazaspiro[2.5]octan-4-yl)-7- |
| | | fluoro-1H-indazole |
| 144 | +++ | 3-(7-(3,5-difluoropyridin-2-yl)-4,7-diazaspiro[2.5]octan-4-yl)- |
| | | 4,7-difluoro-1-((1-(isopropylsulfonyl)-1H-pyrrol-3-yl)sulfonyl)- |
| | | 1H-indazole |
| 145 | +++ | 1-((1-(cyclopropylsulfonyl)-1H-pyrrol-3-yl)sulfonyl)-3-(7-(3,5- |
| | | difluoropyridin-2-yl)-4,7-diazaspiro[2.5]octan-4-yl)-4,7- |
| | | difluoro-1H-indazole |
| 146 | +++ | 3-(7-(5-chloro-3-fluoropyridin-2-yl)-4,7-diazaspiro[2.5]octan-4- |
| | | yl)-1-((1-(cyclopropylsulfonyl)-1H-pyrrol-3-yl)sulfonyl)-4,7- |
| | | difluoro-1H-indazole |
| 147 | +++ | 4-chloro-3-(7-(5-chloro-3-fluoropyridin-2-yl)-4,7- |
| | | diazaspiro[2.5]octan-4-yl)-7-fluoro-1-((1-(isopropylsulfonyl)- |
| | | 1H-pyrrol-3-yl)sulfonyl)-1H-indazole |
| 148 | +++ | 3-[7-(5-chloro-3-fluoro-2-pyridyl)-4,7-diazaspiro [2.5] octan-4- |
| | | yl]-4,7-difluoro-1-(1-isopropylsulfonylpyrrol-3-yl) sulfonyl- |
| | | indazole |
| 149 | +++ | 4,7-difluoro-3-(7-(5-fluoropyrimidin-2-yl)-4,7-diazaspiro [2.5] |
| | | octan-4-yl)-1-((1-(isopropylsulfonyl)-1H-pyrrol-3-yl) sulfonyl)- |
| | | 1H-indazole |
| 150 | +++ | 4-chloro-3-(7-(5-chloro-3-fluoropyridin-2-yl)-4,7-diazaspiro |
| | | [2.5] octan-4-yl)-6-fluoro-1-((1-(isopropylsulfonyl)-1H-pyrrol- |
| | | 3-yl) sulfonyl)-1H-indazole |
| 151 | +++ | 4-chloro-3-[7-(5-chloro-3-fluoro-2-pyridyl)-4,7-diazaspiro |
| | | [2.5] octan-4-yl]-1-(1-isopropylsulfonylpyrrol-3-yl) sulfonyl- |
| | | pyrazolo [3,4-c] pyridine |
| 152 | +++ | 4-chloro-3-[7-(5-chloro-3-fluoro-2-pyridyl)-4,7-diazaspiro [2.5] |
| | | octan-4-yl]-1-(1-isopropylsulfonylpyrrol-3-yl) sulfonyl-pyrazolo |
| | | [3,4-b] pyridine |
| 153 | +++ | 7-chloro-3-[7-(5-chloro-3-fluoro-2-pyridyl)-4,7-diazaspiro [2.5] |
| | | octan-4-yl]-1-(1-isopropylsulfonylpyrrol-3-yl) sulfonyl-pyrazolo |
| | | [3,4-c] pyridine |
| 154 | +++ | 4-chloro-3-(7-(5-chloropyrimidin-2-yl)-4,7- |
| | | diazaspiro[2.5]octan-4-yl)-6-fluoro-1-((1-(isopropylsulfonyl)- |
| | | 1H-pyrrol-3-yl)sulfonyl)-1H-indazole |
| 155 | +++ | 4-chloro-6-fluoro-3-(7-(5-fluoropyrimidin-2-yl)-4,7- |
| | | diazaspiro[2.5]octan-4-yl)-1-((1-(isopropylsulfonyl)-1H-pyrrol- |
| | | 3-yl)sulfonyl)-1H-indazole |
| 156 | +++ | 3-chloro-5-(4-(4-chloro-6-fluoro-1-((1-(isopropylsulfonyl)-1H- |
| | | pyrrol-3-yl)sulfonyl)-1H-indazol-3-yl)-4,7-diazaspiro[2.5]octan- |
| | | 7-yl)-1,2,4-thiadiazole |
| 157 | +++ | 5-chloro-2-(4-(4-chloro-6-fluoro-1-((1-(isopropylsulfonyl)-1H- |
| | | pyrrol-3-yl)sulfonyl)-1H-indazol-3-yl)-4,7-diazaspiro[2.5]octan- |
| | | 7-yl)thiazole |
| 158 | +++ | 4-chloro-3-(7-(3,5-difluoropyridin-2-yl)-4,7- |
| | | diazaspiro[2.5]octan-4-yl)-6-fluoro-1-((1-(isopropylsulfonyl)- |
| | | 1H-pyrrol-3-yl)sulfonyl)-1H-indazole |
| 159 | +++ | 3-(7-(3,5-difluoropyridin-2-yl)-4,7-diazaspiro[2.5]octan-4-yl)- |
| | | 4,6-difluoro-1-((1-(isopropylsulfonyl)-1H-pyrrol-3-yl)sulfonyl)- |
| | | 1H-indazole |
| 160 | +++ | 4-chloro-3-(7-(3,5-difluoropyridin-2-yl)-4,7- |
| | | diazaspiro[2.5]octan-4-yl)-6-fluoro-1-((1-(isopropylsulfonyl)- |
| | | 2,5-dimethyl-1H-pyrrol-3-yl)sulfonyl)-1H-indazole |
| 161 | +++ | 4-chloro-6-fluoro-3-(7-(5-fluoropyridin-2-yl)-4,7- |
| | | diazaspiro[2.5]octan-4-yl)-1-((1-(isopropylsulfonyl)-1H-pyrrol- |
| | | 3-yl)sulfonyl)-1H-indazole |
| 162 | +++ | 4-chloro-6-fluoro-3-(7-(3-fluoropyridin-2-yl)-4,7- |
| | | diazaspiro[2.5]octan-4-yl)-1-((1-(isopropylsulfonyl)-1H-pyrrol- |
| | | 3-yl)sulfonyl)-1H-indazole |
| 163 | +++ | 4-chloro-6-fluoro-1-(1-isopropylsulfonylpyrrol-3-yl)sulfonyl-3- |
| | | [7-(2-pyridyl)-4,7-diazaspiro[2.5]octan-4-yl]indazole |
| 164 | +++ | 4-chloro-6-fluoro-1-(1-isopropylsulfonylpyrrol-3-yl)sulfonyl-3- |
| | | (7-pyrimidin-2-yl-4,7-diazaspiro[2.5]octan-4-yl)indazole |
| 165 | +++ | 4,6-difluoro-3-(7-(5-fluoropyridin-2-yl)-4,7-diazaspiro [2.5] |
| | | octan-4-yl)-1-((1-(isopropylsulfonyl)-1H-pyrrol-3-yl) sulfonyl)- |
| | | 1H-indazole |
| 166 | +++ | 4,6-difluoro-3-(7-(5-fluoropyrimidin-2-yl)-4,7-diazaspiro [2.5] |
| | | octan-4-yl)-1-((1-(isopropylsulfonyl)-1H-pyrrol-3-yl) sulfonyl)- |
| | | 1H-indazole |
| 167 | +++ | 6-chloro-4-fluoro-3-(7-(5-fluoropyrimidin-2-yl)-4,7-diazaspiro |
| | | [2.5] octan-4-yl)-1-((1-(isopropylsulfonyl)-1H-pyrrol-3-yl) |
| | | sulfonyl)-1H-indazole |
| 168 | +++ | 6-chloro-3-[7-(3,5-difluoro-2-pyridyl)-4,7-diazaspiro [2.5] |
| | | octan-4-yl]-4-fluoro-1-(1-isopropylsulfonylpyrrol-3-yl) sulfonyl- |
| | | indazole |
| 169 | +++ | 3-[4,6-difluoro-3-[7-(5-fluoropyrimidin-2-yl)-4,7-diazaspiro |
| | | [2.5] octan-4-yl] indazol-1-yl] sulfonyl-N,N-dimethyl-pyrrole- |
| | | 1-sulfonamide |
| 170 | +++ | 3-[4-chloro-6-fluoro-3-[7-(5-fluoropyrimidin-2-yl)-4,7- |
| | | diazaspiro [2.5] octan-4-yl] indazol-1-yl] sulfonyl-N,N- |
| | | dimethyl-pyrrole-1-sulfonamide |
| 171 | +++ | 1-((1-(tert-butylsulfonyl)-1H-pyrrol-3-yl)sulfonyl)-4-chloro-6- |
| | | fluoro-3-(7-(5-fluoropyrimidin-2-yl)-4,7-diazaspiro[2.5]octan-4- |
| | | yl)-1H-indazole |
| 172 | +++ | 1-((1-(tert-butylsulfonyl)-1H-pyrrol-3-yl) sulfonyl)-4,6- |
| | | difluoro-3-(7-(5-fluoropyrimidin-2-yl)-4,7-diazaspiro [2.5] |
| | | octan-4-yl)-1H-indazole |
| 173 | +++ | 4-chloro-1-((1-(cyclopropylsulfonyl)-1H-pyrrol-3-yl) sulfonyl)- |
| | | 6-fluoro-3-(7-(5-fluoropyrimidin-2-yl)-4,7-diazaspiro [2.5] |
| | | octan-4-yl)-1H-indazole |
| 174 | +++ | 1-((1-(cyclopropylsulfonyl)-1H-pyrrol-3-yl) sulfonyl)-4,6- |
| | | difluoro-3-(7-(5-fluoropyrimidin-2-yl)-4,7-diazaspiro [2.5] |
| | | octan-4-yl)-1H-indazole |
| 175 | +++ | 4-chloro-6-fluoro-3-(7-(4-fluorophenyl)-4,7-diazaspiro [2.5] |
| | | octan-4-yl)-1-((1-(isopropylsulfonyl)-1H-pyrrol-3-yl) sulfonyl)- |
| | | 1H-indazole |
| 176 | + | 4-chloro-6-fluoro-1-(1-isopropylsulfonylpyrrol-3-yl) sulfonyl-3- |
| | | [7-(4-pyridyl)-4,7-diazaspiro [2.5] octan-4-yl] indazole |
| 177 | +++ | 4-chloro-6-fluoro-3-[(2S)-4-(5-fluoropyrimidin-2-yl)-2-methyl- |
| | | piperazin-1-yl]-1-(1-isopropylsulfonylpyrrol-3-yl) sulfonyl- |
| | | indazole |
| 178 | +++ | 4-chloro-6-fluoro-3-[(2R)-4-(5-fluoropyrimidin-2-yl)-2-methyl- |
| | | piperazin-1-yl]-1-(1-isopropylsulfonylpyrrol-3-yl) sulfonyl- |
| | | indazole |
| 179 | +++ | 4-chloro-6-fluoro-3-(8-(5-fluoropyrimidin-2-yl)-5,8-diazaspiro |
| | | [3.5] nonan-5-yl)-1-((1-(isopropylsulfonyl)-1H-pyrrol-3-yl) |
| | | sulfonyl)-1H-indazole |
| 180 | +++ | 4-chloro-6-fluoro-3-[7-(5-fluoropyrimidin-2-yl)-4,7- |
| | | diazaspiro[2.5]octan-4-yl]-1-(2,4,5-trideuterio-1- |
| | | isopropylsulfonyl-pyrrol-3-yl)sulfonyl-indazole |
| 181 | +++ | 4-chloro-1-[1-(1-chloro-1-methyl-ethyl)sulfonyl-2,4,5- |
| | | trideuterio-pyrrol-3-yl]sulfonyl-6-fluoro-3-[7-(5- |
| | | fluoropyrimidin-2-yl)-4,7-diazaspiro[2.5]octan-4-yl]indazole |
| 182 | +++ | 4-chloro-3-(7-(2,6-difluorophenyl)-4,7-diazaspiro [2.5] octan- |
| | | 4-yl)-6-fluoro-1-((1-(isopropylsulfonyl)-1H-pyrrol-3-yl) |
| | | sulfonyl)-1H-indazole |
| 183 | ++ | 4-chloro-6-fluoro-1-((1-(isopropylsulfonyl)-1H-pyrrol-3- |
| | | yl)sulfonyl)-3-(7-(methylsulfonyl)-4,7-diazaspiro[2.5]octan-4- |
| | | yl)-1H-indazole |
| 184 | +++ | 4-chloro-6-fluoro-1-((1-(isopropylsulfonyl)-1H-pyrrol-3- |
| | | yl)sulfonyl)-3-(2-(trifluoromethyl)pyrrolidin-1-yl)-1H-indazole |
| 185 | +++ | 4-chloro-6-fluoro-3-(4-(5-fluoropyrimidin-2-yl)-2,2- |
| | | dimethylpiperazin-1-yl)-1-((1-(isopropylsulfonyl)-1H-pyrrol-3- |
| | | yl)sulfonyl)-1H-indazole |
| 186 | ++ | 4-(4-chloro-6-fluoro-1-((1-(isopropylsulfonyl)-1H-pyrrol-3- |
| | | yl)sulfonyl)-1H-indazol-3-yl)-N,N-dimethyl-4- |
| | | azaspiro[2.5]octan-7-amine |
| 187 | +++ | 4-(4-chloro-6-fluoro-1-((1-(isopropylsulfonyl)-1H-pyrrol-3- |
| | | yl)sulfonyl)-1H-indazol-3-yl)-4-azaspiro[2.5]octan-7-one |
| 188 | +++ | 4-chloro-6-fluoro-1-((1-(isopropylsulfonyl)-1H-pyrrol-3- |
| | | yl)sulfonyl)-3-(4-azaspiro[2.4]heptan-4-yl)-1H-indazole |
| 189 | +++ | 4-chloro-6-fluoro-1-((1-(isopropylsulfonyl)-1H-pyrrol-3- |
| | | yl)sulfonyl)-3-(1-azaspiro[3.3]heptan-1-yl)-1H-indazole |
| 190 | +++ | 4-(4-chloro-6-fluoro-1-((1-(isopropylsulfonyl)-1H-pyrrol-3- |
| | | yl)sulfonyl)-1H-indazol-3-yl)-7-oxa-4-azaspiro[2.5]octane |
| 191 | +++ | 4-chloro-3-(7,7-difluoro-4-azaspiro[2.5]octan-4-yl)-6-fluoro-1- |
| | | ((1-(isopropylsulfonyl)-1H-pyrrol-3-yl)sulfonyl)-1H-indazole |
| 192 | +++ | 4-chloro-6-fluoro-3-(7-fluoro-4-azaspiro[2.5]octan-4-yl)-1-((1- |
| | | (isopropylsulfonyl)-1H-pyrrol-3-yl)sulfonyl)-1H-indazole |
| 193 | ++ | 4-((3-(7-(5-chloro-3-fluoropyridin-2-yl)-4,7- |
| | | diazaspiro[2.5]octan-4-yl)-4,7-difluoro-1H-indazol-1- |
| | | yl)sulfonyl)-2-(isopropylsulfonyl)thiazole |
| 194 | ++ | 4-((3-(7-(3,5-difluoropyridin-2-yl)-4,7-diazaspiro[2.5]octan-4- |
| | | yl)-4,7-difluoro-1H-indazol-1-yl)sulfonyl)-2- |
| | | (isopropylsulfonyl)thiazole |
| 195 | ++ | 4-((4,7-difluoro-3-(7-(5-fluoropyrimidin-2-yl)-4,7- |
| | | diazaspiro[2.5]octan-4-yl)-1H-indazol-1-yl)sulfonyl)-2- |
| | | (isopropylsulfonyl)thiazole |
| 196 | +++ | 4-((4-chloro-3-(7-(5-chloro-3-fluoropyridin-2-yl)-4,7- |
| | | diazaspiro[2.5]octan-4-yl)-1H-indazol-1-yl)sulfonyl)-2- |
| | | (isopropylsulfonyl)thiazole |
| 197 | ++ | 4-((4-chloro-3-(7-(3,5-difluoropyridin-2-yl)-4,7- |
| | | diazaspiro[2.5]octan-4-yl)-6-fluoro-1H-indazol-1-yl)sulfonyl)-2- |
| | | (isopropylsulfonyl)thiazole |
| 198 | ++ | 4-((3-(7-(3,5-difluoropyridin-2-yl)-4,7-diazaspiro[2.5]octan-4- |
| | | yl)-4,6-difluoro-1H-indazol-1-yl)sulfonyl)-2- |
| | | (isopropylsulfonyl)thiazole |
| 199 | +++ | 4-chloro-3-(7-(3,5-difluoropyridin-2-yl)-4,7- |
| | | diazaspiro[2.5]octan-4-yl)-6-fluoro-1-((1-(isopropylsulfonyl)- |
| | | 1H-pyrazol-3-yl)sulfonyl)-1H-indazole |
| 200 | + | 3-(7-(3,5-difluoropyridin-2-yl)-4,7-diazaspiro[2.5]octan-4-yl)- |
| | | 4,6-difluoro-1-((1-(isopropylsulfonyl)-1H-1,2,4-triazol-3- |
| | | yl)sulfonyl)-1H-indazole |

*EC₅₀ of TFEB activation (where data is provided): “+++” ≤1 uM; “++” 1 uM to 10 uM; “+” ≥10 uM

### METHODS OF SYNTHESIS

The following examples are given for the purpose of illustrating the invention, but not for limiting the scope or spirit of the invention.

Compounds of the invention, including those specifically disclosed herein above and herein below, may be prepared as described in the following schemes. For example, the compounds of Formula I may be prepared as described in Schemes below, which are known to those of skill in the art for making fragments and combinations thereof.

### Intermediate-L1: (1Z)-2, 6-dichloro-N-(p-tolylsulfonyl) benzohydrazonoyl chloride

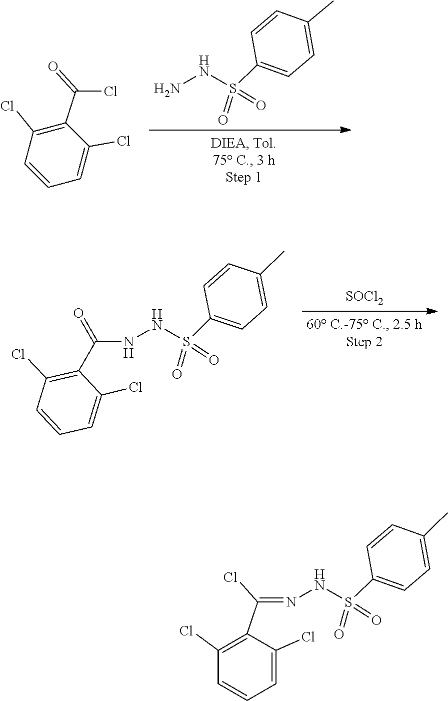

Step 1: To a solution of 2, 6-dichlorobenzoyl chloride (5 g, 23.9 mmol, 3.42 mL, 1 eq) in toluene (100 mL) was added 4-methylbenzenesulfonohydrazide (4.22 g, 22.7 mmol, 0.95 eq) and diisopropylethylamine (3.70 g, 28.7 mmol, 4.99 mL, 1.2 eq). The mixture was stirred at 75° C. for 3 hours. LC-MS showed 2, 6-dichlorobenzoyl chloride was consumed completely and one main peak with desired mass was detected. The reaction mixture was filtered, and the filter cake was dried in vacuum to give desired 2, 6-dichloro-N′-(p-tolylsulfonyl) benzohydrazide (5.5 g, crude) as a yellow solid. MS (ESI): mass calcd. For C₁₄H₁₂C₁₂N₂O₃S 357.99, m/z found 359.1 [M+H]⁺.

Step 2: To a solution of 2, 6-dichloro-N′-(p-tolylsulfonyl) benzohydrazide (1.6 g, 4.45 mmol, 1 eq) and thionyl chloride (6.09 g, 51.2 mmol, 3.72 mL, 11.5 eq) was stirred at 75° C. for 1.5 hours. The reaction was cooled to 60° C. and an additional portion of 2, 6-dichloro-N′-(p-tolylsulfonyl) benzohydrazide (1.60 g, 4.45 mmol, 1 eq) was added and the reaction heated back to 75° C. for 1 hour. LC-MS (the simple was quenched with piperidine) showed 2, 6-dichloro-N′-(p-tolylsulfonyl) benzohydrazide was consumed completely and desired mass was detected. The reaction mixture was concentrated to give desired (1Z)-2, 6-dichloro-N-(p-tolylsulfonyl) benzohydrazonoyl chloride (1.7 g, crude) as a yellow gum. MS (ESI): mass calcd. For C₁₄H₁₁C₁₃N₂O₂S 375.96, m/z found 426.1 [M+H+50]⁺.

### Intermediate-L2: N′-(chloro (2, 6-dichloro-4-fluorophenyl) methyl)-4-methylbenzenesulfonohydrazide

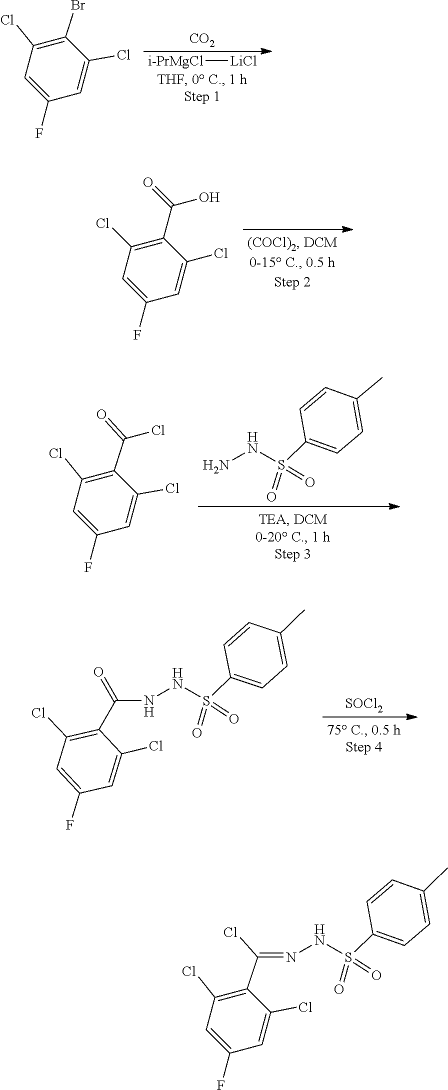

Step 1: To a solution of 2-bromo-1, 3-dichloro-5-fluoro-benzene (12 g, 49.2 mmol, 0.67 eq) in THF (120 mL) was added dropwise i-PrMgCl—LiCl (1.3 M, 56.5 mL, 1 eq) at 0° C. After addition, the mixture was stirred at this temperature for 0.5 hour, and then CO₂ (15 psi) was added dropwise at 0° C. The resulting mixture was stirred at 0° C. for 0.5 hour. TLC (petroleum ether/EtOAc=4/1) indicated 2-bromo-1, 3-dichloro-5-fluoro-benzene was consumed completely and one new spot formed. The crude was added H₂O (100 mL) and extracted with EtOAc (150 mL*3). The combined organic layers were washed with brine (100 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜20% EtOAc/petroleum ether gradient @100 mL/min) to give desired 2, 6-dichloro-4-fluorobenzoic acid (5.3 g, 25.4 mmol, 34.5% yield) as a yellow solid.

Step 2: To a solution of 2, 6-dichloro-4-fluoro-benzoic acid (5.3 g, 25.4 mmol, 1 eq) in DCM (50 mL) was added (COCl)₂ (6.44 g, 50.7 mmol, 4.44 mL, 2 eq) and DMF (92.7 mg, 1.27 mmol, 97.6 μL, 0.05 eq) at 0° C. The mixture was stirred at 15° C. for 0.5 hour. TLC (petroleum ether/EtOAc=3/1) indicated 2-bromo-1, 3-dichloro-5-fluoro-benzene was consumed completely and one new spot formed. The reaction mixture was concentrated under reduced pressure to give desired 2, 6-dichloro-4-fluorobenzoyl chloride (5.7 g, crude) as a brown solid.

Step 3: To a solution of 2, 6-dichloro-4-fluoro-benzoyl chloride (5.7 g, 25.1 mmol, 1 eq) and TEA (6.34 g, 62.7 mmol, 8.72 mL, 2.5 eq) in DCM (60 mL) was added 4-methylbenzenesulfonohydrazide (6.07 g, 32.6 mmol, 1.3 eq) at 0° C. The mixture was stirred at 20° C. for 1 hour. TLC (petroleum ether/EtOAc=3/1) indicated 2, 6-dichloro-4-fluoro-benzoyl chloride was consumed completely and one new spot formed. The crude was added H₂O (100 mL) and extracted with EtOAc (150 mL*3). The combined organic layers were washed with brine (100 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜35% EtOAc/petroleum ether gradient @100 mL/min) to give desired N′-(2, 6-dichloro-4-fluorobenzoyl)-4-methylbenzenesulfonohydrazide (5.5 g, 14.6 mmol, 58.2% yield) as a yellow solid.

Step 4: To a solution of 2, 6-dichloro-4-fluoro-N′-(p-tolylsulfonyl) benzohydrazide (3.5 g, 9.28 mmol, 1 eq) in SOCl₂ (40 mL). The mixture was stirred at 75° C. for 0.5 hour. LC-MS (The simple was quenched with piperidine) showed 2, 6-dichloro-4-fluoro-N′-(p-tolylsulfonyl) benzohydrazide was consumed completely and desired mass was detected. The reaction mixture was concentrated under reduced pressure to give desired N′-(chloro (2, 6-dichloro-4-fluorophenyl) methyl)-4-methylbenzenesulfonohydrazide (3.5 g, crude) as a brown solid. MS (ESI): mass calcd. For C₁₄H₁₂C₁₃FN₂O₂S 393.95, m/z found 444.0 [M+H+50]⁺.

### Intermediate-L3: 7-(5-fluoropyrimidin-2-yl)-4, 7-diazaspiro[2.5]octane

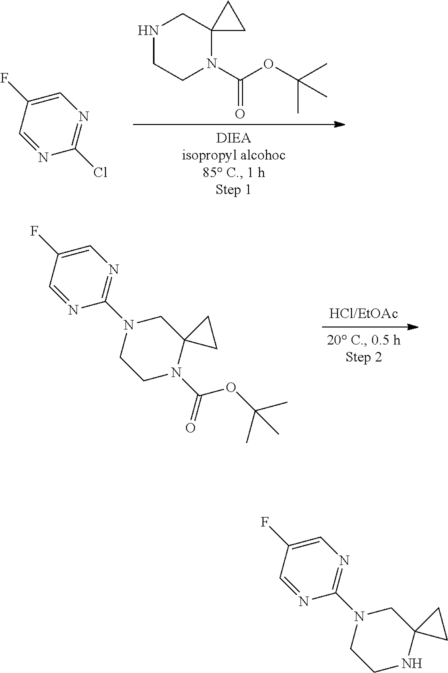 [SMILES: Fc1cnc(Cl)nc1 ; CC(C)(C)OC(=O)N1CCNCC12CC2 ; CC(C)(C)OC(=O)N1CCN(c2ncc(F)cn2)CC12CC2 ; Fc1cnc(N2CCNC3(CC3)C2)nc1]

Step 1: To a solution of 2-chloro-5-fluoro-pyrimidine (1.3 g, 9.81 mmol, 1.21 mL, 1 eq) and tert-butyl 4, 7-diazaspiro [2.5]octane-4-carboxylate (2.08 g, 9.81 mmol, 1 eq) in isopropyl alcohol (20 mL) was added DIEA (2.79 g, 21.6 mmol, 3.76 mL, 2.2 eq). The mixture was stirred at 85° C. for 1 hour. LC-MS showed a little 2-chloro-5-fluoro-pyrimidine remained and desired compound was detected. The crude was added H₂O (50 mL) and extracted with EtOAc (50 mL*3). The combined organic layers were washed with brine (50 mL*2), dried over anhydrous Na₂SO₄, filtered and concentrated under reduced pressure to give a residue. The residue was purified by prep-TLC (SiO₂, petroleum ether/EtOAc=5/1) to give desired tert-butyl 7-(5-fluoropyrimidin-2-yl)-4, 7-diazaspiro [2.5]octane-4-carboxylate (2 g, 6.49 mmol, 66.1% yield) as a yellow oil. MS (ESI): mass calcd. For C₁₅H₂₁FN₄O₂ 308.16, m/z found 253.2 [M+H−56]⁺.

Step 2: The solution of tert-butyl 7-(5-fluoropyrimidin-2-yl)-4, 7-diazaspiro [2.5]octane-4-carboxylate (2 g, 6.49 mmol, 1 eq) in HCl/EtOAc (4 M, 30 mL) was stirred at 20° C. for 0.5 hour. TLC (SiO₂, petroleum ether/EtOAc=5/1) showed 4-(3-fluoro-2-pyridyl)-4, 7-diazaspiro [2.5]octane-7-carboxylate was consumed completely and one new spot formed. The reaction mixture was concentrated to give desired 7-(5-fluoropyrimidin-2-yl)-4, 7-diazaspiro [2.5]octane (1.5 g, crude, HCl) as a yellow solid.

### Intermediate-L4: 7-(5-chloropyrimidin-2-yl)-4, 7-diazaspiro [2.5]octane

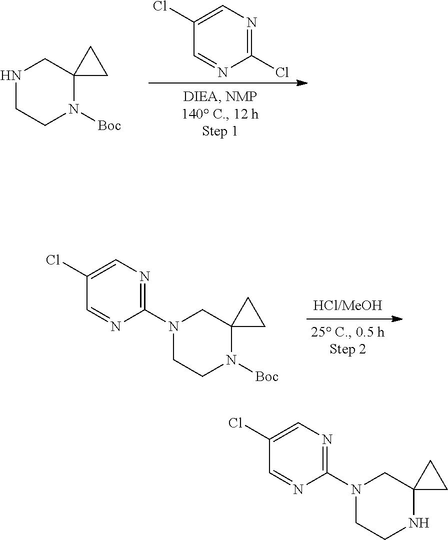 [SMILES: CC(C)(C)OC(=O)N1CCNCC12CC2 ; Clc1cnc(Cl)nc1 ; CC(C)(C)OC(=O)N1CCN(c2ncc(Cl)cn2)CC12CC2 ; Clc1cnc(N2CCNC3(CC3)C2)nc1]

Step 1: To a solution of tert-butyl 4, 7-diazaspiro [2.5]octane-4-carboxylate (1.5 g, 7.07 mmol, 1.1 eq) in NMP (20 mL) was added DIEA (1.25 g, 9.64 mmol, 1.68 mL, 1.5 eq) and 2, 5-dichloropyrimidine (957 mg, 6.42 mmol, 1 eq). The mixture was stirred at 140° C. for 12 hours. LC-MS showed tert-butyl 4, 7-diazaspiro [2.5]octane-4-carboxylate was consumed completely and desired mass was detected. The reaction mixture was added to water (50 mL), extracted with EtOAc (100 mL*3). The combined organic layers were washed with brine 20 mL and dried over Na₂SO₄, filtered and concentrated under reduced pressure to give a residue. The residue was purified by column chromatography (SiO₂, petroleum ether/EtOAc=1/0 to 1/1) to give desired tert-butyl 7-(5-chloropyrimidin-2-yl)-4, 7-diazaspiro [2.5]octane-4-carboxylate (1.8 g, crude) as a white solid. MS (ESI): mass calcd. For C₁₅H₂₁ClN₄O₂ 324.14, m/z found 268.9 [M+H−56]⁺.

Step 2: A mixture of tert-butyl 7-(5-chloropyrimidin-2-yl)-4, 7-diazaspiro [2.5]octane-4-carboxylate (1.98 g, 6.10 mmol, 1 eq) in HCl/MeOH (4 M, 20 mL) was stirred at 25° C. for 0.5 hour. LC-MS showed a little of tert-butyl 7-(5-chloropyrimidin-2-yl)-4, 7-diazaspiro [2.5]octane-4-carboxylate remained and desired mass was detected. The reaction mixture was concentrated to give desired 7-(5-chloropyrimidin-2-yl)-4, 7-diazaspiro [2.5]octane (1.8 g, crude, HCl) as a white solid. MS (ESI): mass calcd. For C₁₀H₁₃ClN₄ 224.08, m/z found 225.2 [M+H]⁺.

### Intermediate-L5: (1Z)-2, 4, 6-trifluoro-N-(p-tolylsulfonyl) benzohydrazonoyl chloride

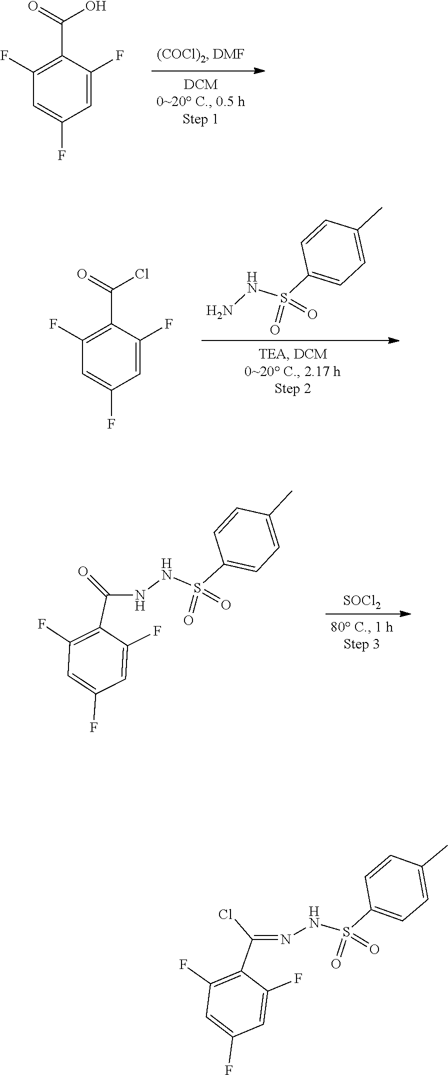 [SMILES: O=C(O)c1c(F)cc(F)cc1F ; O=C(Cl)c1c(F)cc(F)cc1F ; Cc1ccc(S(=O)(=O)NN)cc1 ; Cc1ccc(S(=O)(=O)NNC(=O)c2c(F)cc(F)cc2F)cc1 ; "SOCl2" ; Cc1ccc(S(=O)(=O)N/N=C(\Cl)c2c(F)cc(F)cc2F)cc1]

Step 1: To a solution of 2, 4, 6-trifluorobenzoic acid (7.5 g, 42.6 mmol, 1 eq) and DMF (31 mg, 4.26 mmol, 328 μL, 0.1 eq) in DCM (75 mL) was added (COCl)₂ (10.8 g, 85.2 mmol, 7.46 mL, 2 eq) at 0° C. The mixture was stirred at 20° C. for 0.5 hour. TLC (petroleum ether/EtOAc=3/1) indicated 2, 4, 6-trifluorobenzoic acid was consumed completely and one new spot formed. The reaction mixture was concentrated in vacuum to give desired 2, 4, 6-trifluorobenzoyl chloride (17 g, crude) as a yellow solid.

Step 2: To a solution of 4-methylbenzenesulfonohydrazide (7.18 g, 38.6 mmol, 1.5 eq) in DCM (90 mL) was added TEA (6.50 g, 64.3 mmol, 8.94 mL, 2.5 eq) at 20° C. After addition, the mixture was stirred at this temperature for 10 minutes, and then 2, 4, 6-trifluorobenzoyl chloride (5 g, 25.7 mmol, 1.0 eq) in DCM (10 mL) was added dropwise at 0° C. The resulting mixture was stirred at 20° C. for 2 hours. LC-MS showed 4-methylbenzenesulfonohydrazide was consumed completely and one main peak with desired mass was detected. The residue was diluted with H₂O (100 mL) and extracted with DCM (50 mL*3). The combined organic layers were washed with brine (100 mL), dried over Na₂SO₄, filtered and concentrated under reduced pressure to give a residue. The residue was purified by flash silica gel chromatography (ISCO®; 220 g SepaFlash® Silica Flash Column, Eluent of 0˜35% EtOAc/petroleum ether gradient @100 mL/min) to give desired 2, 4, 6-trifluoro-N′-(p-tolylsulfonyl) benzohydrazide (4 g, crude) as a yellow solid. MS (ESI): mass calcd. For C₁₄H₁₁F₃N₂O₃S 344.04 m/z found 345.2 [M+H]⁺.

Step 3: A mixture of 2, 4, 6-trifluoro-N′-(p-tolylsulfonyl) benzohydrazide (0.7 g, 2.03 mmol, 1 eq) in SOCl₂ (6 mL) was degassed and purged with N₂ for 3 times, and then the mixture was stirred at 80° C. for 1 hour under the atmosphere of nitrogen. TLC (petroleum ether/EtOAc=3/1) indicated 2, 4, 6-trifluoro-N′-(p-tolylsulfonyl) benzohydrazide was consumed completely and two new spots formed. The reaction mixture was concentrated in vacuum to give desired (1Z)-2, 4, 6-trifluoro-N-(p-tolylsulfonyl) benzohydrazonoyl chloride (0.7 g, crude) as a yellow solid.

### Intermediate-L6: 4-chloro-3-[7-(5-chloropyrimidin-2-yl)-4, 7-diazaspiro [2.5]octan-4-yl]-1H-indazole

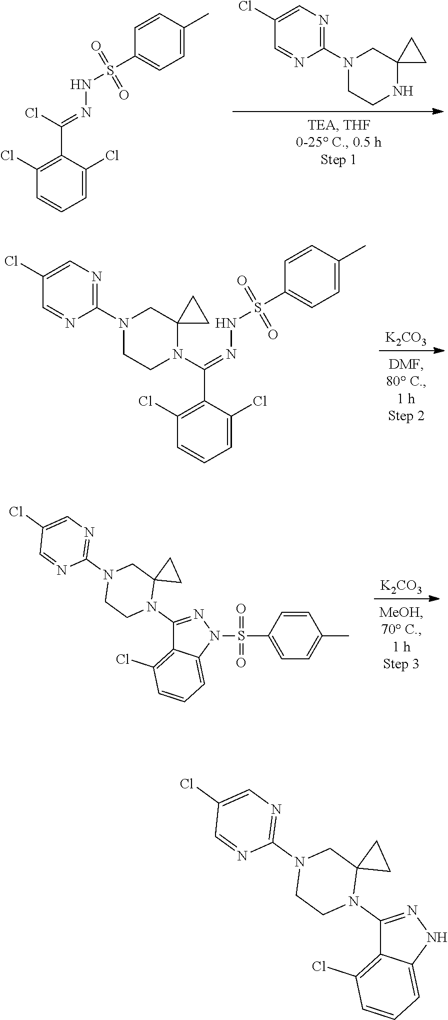

Step 1: To a solution of 7-(5-chloropyrimidin-2-yl)-4,7-diazaspiro[2.5]octane (251 mg, 963 μmol, 1 eq, HCl) in THF (5 mL) was added dropwise TEA (974 mg, 9.63 mmol, 1.34 mL, 10 eq) at 25° C. After addition, the mixture was stirred at this temperature for 10 minutes, and then (1Z)-2,6-dichloro-N-(p-tolylsulfonyl)benzohydrazonoyl chloride (400 mg, 1.06 mmol, 1.1 eq) in THF (2 mL) was added dropwise at 0° C. The resulting mixture was stirred at 25° C. for 20 minutes. LC-MS showed 7-(5-chloropyrimidin-2-yl)-4, 7-diazaspiro [2.5]octane was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(Z)-[[7-(5-chloropyrimidin-2-yl)-4, 7-diazaspiro[2.5]octan-4-yl]-(2,6-dichlorophenyl)methylene]amino]-4-methyl-benzenesulfonamide (520 mg, crude) as a yellow solid. MS (ESI): mass calcd. For C₂₄H₂₃C₁₃N₆O₂S 564.07, m/z found 565.1 [M+H]⁺.

Step 2: To a solution of N—[(Z)—[[7-(5-chloropyrimidin-2-yl)-4, 7-diazaspiro [2.5]octan-4-yl]-(2, 6-dichlorophenyl) methylene]amino]-4-methyl-benzenesulfonamide (520 mg, 919 μmol, 1 eq) in DMF (10 mL) was added K₂CO₃ (508 mg, 3.68 mmol, 4 eq). The mixture was stirred at 80° C. for 1 hour. LC-MS showed N—[(Z)—[[7-(5-chloropyrimidin-2-yl)-4, 7-diazaspiro [2.5]octan-4-yl]-(2, 6-dichlorophenyl) methylene]amino]-4-methyl-benzenesulfonamide was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give desired 4-chloro-3-[7-(5-chloropyrimidin-2-yl)-4,7-diazaspiro[2.5]octan-4-yl]-1-(p-tolylsulfonyl)indazole (500 mg, crude) as a black oil. MS (ESI): mass calcd. For C₂₄H₂₂C₁₂N₆O₂S 528.09, m/z found 529.1 [M+H]⁺.

Step 3: To a solution of 4-chloro-3-[7-(5-chloropyrimidin-2-yl)-4, 7-diazaspiro [2.5]octan-4-yl]-1-(p-tolylsulfonyl) indazole (500 mg, 944 μmol, 1 eq) in MeOH (10 mL) was added K₂CO₃ (652 mg, 4.72 mmol, 5 eq). The mixture was stirred at 70° C. for 1 hour. LC-MS showed 4-chloro-3-[7-(5-chloropyrimidin-2-yl)-4, 7-diazaspiro [2.5]octan-4-yl]-1-(p-tolylsulfonyl) indazole was consumed completely and desired compound was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by column chromatography (SiO₂, petroleum ether/Ethyl acetate=1/0 to 1/1) to give desired 4-chloro-3-[7-(5-chloropyrimidin-2-yl)-4, 7-diazaspiro [2.5]octan-4-yl]-1H-indazole (150 mg, crude) as a yellow oil. MS (ESI): mass calcd. For C₁₇H₁₆C₁₂N₆ 374.08, m/z found 375.1 [M+H]⁺.

### Intermediate-L7: 4-chloro-6-fluoro-3-(7-(5-fluoropyrimidin-2-yl)-4, 7-diazaspiro [2.5]octan-4-yl)-1H-indazole

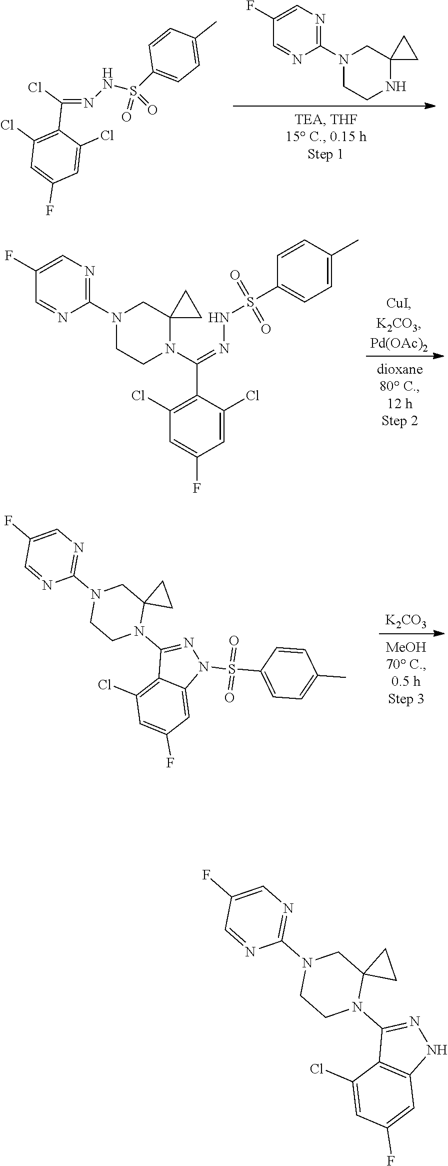

Step 1: To a solution of N′-[chloro-(2,6-dichloro-4-fluoro-phenyl)methyl]-4-methyl-benzenesulfonohydrazide (600 mg, 1.51 mmol, 1.1 eq) in THF (10 mL) was added TEA (278 mg, 2.74 mmol, 382 μL, 2 eq) and 7-(5-fluoropyrimidin-2-yl)-4, 7-diazaspiro [2.5]octane (336 mg, 1.37 mmol, 1 eq, HCl). The mixture was stirred at 15° C. for 0.15 hour. LC-MS showed N′-[chloro-(2, 6-dichloro-4-fluoro-phenyl) methyl]-4-methyl-benzenesulfonohydrazide was consumed completely and desired mass was detected. The crude was added H₂O (20 mL), and extracted with EtOAc (45 mL). The combined organic layers were washed with brine (10 mL), dried over Na₂SO₄, filtered and concentrated under reduced pressure to give desired (Z)—N′-((2, 6-dichloro-4-fluorophenyl) (7-(5-fluoropyrimidin-2-yl)-4, 7-diazaspiro [2.5]octan-4-yl) methylene)-4-methylbenzenesulfonohydrazide (700 mg, crude) as a yellow solid. MS (ESI): mass calcd. For C₂₄H₂₂C₁₂F₂N₆O₂S 566.09, m/z found 567.0 [M+H]⁺.

Step 2: A mixture of N—[(Z)-[(2,6-dichloro-4-fluoro-phenyl)-[7-(5-fluoropyrimidin-2-yl)-4,7-diazaspiro[2.5]octan-4-yl]methylene]amino]-4-methyl-benzenesulfonamide (700 mg, 1.23 mmol, 1 eq), CuI (23.5 mg, 123 μmol, 0.1 eq), K₂CO₃ (426 mg, 3.08 mmol, 2.5 eq) and Pd(OAc)₂ (55.4 mg, 247 μmol, 0.2 eq) in dioxane (10 mL) was degassed and purged with N₂ for 3 times, and then the mixture was stirred at 80° C. for 12 hours under the atmosphere of nitrogen. LC-MS showed N—[(Z)-[(2, 6-dichloro-4-fluoro-phenyl)-[7-(5-fluoropyrimidin-2-yl)-4, 7-diazaspiro [2.5]octan-4-yl]methylene]amino]-4-methyl-benzenesulfonamide was consumed completely and desired mass was detected. The crude was added H₂O (10 mL), and extracted with EtOAc (45 mL). The combined organic layers were washed with brine (40 mL), dried over Na₂SO₄, filtered and concentrated under reduced pressure to give desired 4-chloro-6-fluoro-3-(7-(5-fluoropyrimidin-2-yl)-4, 7-diazaspiro [2.5]octan-4-yl)-1-tosyl-1H-indazole (400 mg, crude) as a brown oil. MS (ESI): mass calcd. For C₂₄H₂₁ClF₂N₆O₂S 530.11, m/z found 531.0 [M+H]⁺.

Step 3: To a solution of 4-chloro-6-fluoro-3-[7-(5-fluoropyrimidin-2-yl)-4, 7-diazaspiro [2.5]octan-4-yl]-1-(p-tolylsulfonyl) indazole (400 mg, 753 μmol, 1 eq) in MeOH (10 mL) was added K₂CO₃ (520 mg, 3.77 mmol, 5 eq). The mixture was stirred at 70° C. for 0.5 hour. LC-MS showed 4-chloro-6-fluoro-3-[7-(5-fluoropyrimidin-2-yl)-4, 7-diazaspiro [2.5]octan-4-yl]-1-(p-tolylsulfonyl) indazole was consumed completely and desired mass was detected. The reaction mixture was concentrated under reduced pressure to give a residue. The crude was added H₂O (10 mL), and extracted with EtOAc (45 mL). The combined organic layers were washed with brine (10 mL), dried over Na₂SO₄, filtered and concentrated under reduced pressure to give a residue. The residue was purified by prep-HPLC (neutral condition, column: Waters Xbridge Prep OBD C18 150*40 mm*10 um; mobile phase: [water-ACN]; B %: 60%-85%, 8 minutes) to give desired 4-chloro-6-fluoro-3-(7-(5-fluoropyrimidin-2-yl)-4, 7-diazaspiro [2.5]octan-4-yl)-1H-indazole (60 mg, 159.24 μmol, 21.14% yield) as a yellow solid. MS (ESI): mass calcd. For C₁₇H₁₅ClF₂N₆ 376.10, m/z found 377.0 [M+H]⁺.

### Intermediate-L8: 4-chloro-3-[7-(5-fluoropyrimidin-2-yl)-4, 7-diazaspiro [2.5]octan-4-yl]-1H-indazole

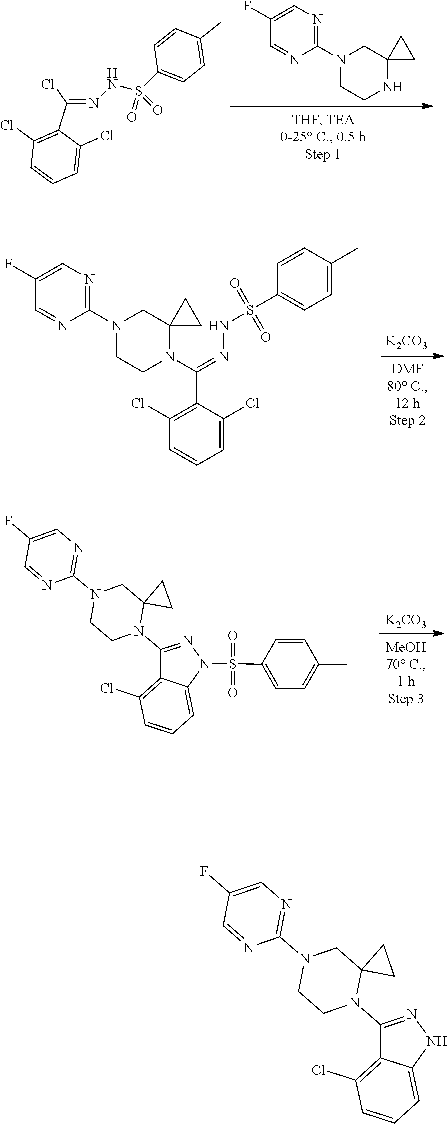

Step 1: To a solution of 7-(5-fluoropyrimidin-2-yl)-4,7-diazaspiro[2.5]octane (778 mg, 3.18 mmol, 1.5 eq, HCl) in THF (5 mL) was added dropwise TEA (2.14 g, 21.2 mmol, 2.95 mL, 10 eq) at 25° C. After addition, the mixture was stirred at this temperature for 10 minutes, and then (1Z)-2,6-dichloro-N-(p-tolylsulfonyl)benzohydrazonoyl chloride (800 mg, 2.12 mmol, 1 eq) in THF (3 mL) was added dropwise at 0° C. The resulting mixture was stirred at 25° C. for 20 minutes. LC-MS showed 7-(5-fluoropyrimidin-2-yl)-4, 7-diazaspiro [2.5]octane was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N—[(Z)-[(2, 6-dichlorophenyl)-[7-(5-fluoropyrimidin-2-yl)-4, 7-diazaspiro [2.5]octan-4-yl]methylene]amino]-4-methyl-benzene sulfonamide (1.35 g, crude) was obtained as a white solid. MS (ESI): mass calcd. For C₂₄H₂₃Cl₂FN₆O₂S 548.10, m/z found 549.1 [M+H]⁺.

Step 2: To a solution of N—[(Z)-[(2, 6-dichlorophenyl)-[7-(5-fluoropyrimidin-2-yl)-4, 7-diazaspiro [2.5]octan-4-yl]methylene]amino]-4-methyl-benzenesulfonamide (1.3 g, 2.37 mmol, 1 eq) in DMF (14 mL) was added K₂CO₃ (1.63 g, 11.8 mmol, 5 eq). The mixture was stirred at 80° C. for 12 hours. LC-MS showed N—[(Z)-[(2, 6-dichlorophenyl)-[7-(5-fluoropyrimidin-2-yl)-4, 7-diazaspiro [2.5]octan-4-yl]methylene]amino]-4-methyl-benzenesulfonamide was consumed completely and desired mass was detected. The reaction mixture was partitioned between 20 mL of H₂O and 20 mL of EtOAc. The organic phase was separated, washed with 50 mL of brine, dried over Na₂SO₄, filtered and concentrated under reduced pressure to give desired 4-chloro-3-[7-(5-fluoropyrimidin-2-yl)-4, 7-diazaspiro [2.5]octan-4-yl]-1-(p-tolylsulfonyl) indazole (1.5 g, crude) as a yellow oil. MS (ESI): mass calcd. For C₂₄H₂₂ClFN₆O₂S 512.12, m/z found 513.0 [M+H]⁺.

Step 3: To a solution of 4-chloro-3-[7-(5-fluoropyrimidin-2-yl)-4, 7-diazaspiro [2.5]octan-4-yl]-1-(p-tolylsulfonyl) indazole (1.5 g, 2.92 mmol, 1 eq) in MeOH (6 mL) was added K₂CO₃ (2.02 g, 14.6 mmol, 5 eq). The mixture was stirred at 70° C. for 1 hour. LC-MS showed 4-chloro-3-[7-(5-fluoropyrimidin-2-yl)-4, 7-diazaspiro [2.5]octan-4-yl]-1-(p-tolylsulfonyl) indazole was consumed completely and desired mass was detected. The reaction mixture was filtered and the filtrate was concentrated under reduced pressure to give a residue. The residue was purified by flash silica gel chromatography (ISCO®; 2 g SepaFlash® Silica Flash Column, Eluent of 0˜28% ethyl acetate/petroleum ether gradient @60 mL/min) to give desired 4-chloro-3-[7-(5-fluoropyrimidin-2-yl)-4, 7-diazaspiro [2.5]octan-4-yl]-1H-indazole (250 mg, 697 μmol, 23.8% yield) as a yellow oil. MS (ESI): mass calcd. For C₁₇H₁₆ClFN₆ 358.11, m/z found 359.1 [M+H]⁺.

### Intermediate-L9: 4-(4-chloro-1-tosyl-1H-indazol-3-yl)-4-azaspiro[2.5]octan-7-one

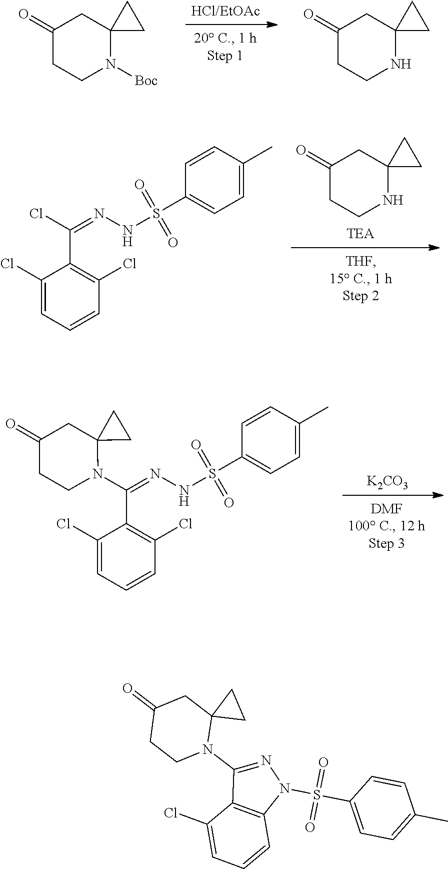

Step 1: To the solution of tert-butyl 7-oxo-4-azaspiro [2.5]octane-4-carboxylate (2 g, 8.88 mmol, 1 eq) in EtOAc (3 mL) was added HCl (4 M, 22.19 mL, 10 eq) at 15° C. and the solution was stirred at 15° C. for 1 hour. TLC showed tert-butyl 7-oxo-4-azaspiro [2.5]octane-4-carboxylate was consumed completely. The reaction mixture was concentrated to give desired 4-azaspiro [2.5]octan-7-one (1.43 g, crude, HCl) as a white solid.

Step 2: To the solution of (1E)-2,6-dichloro-N-(p-tolylsulfonyl)benzohydrazonoyl chloride (3.34 g, 8.85 mmol, 1 eq) in THF (20 mL) was added TEA (8.95 g, 88.5 mmol, 10 eq) at 15° C. and the solution was stirred at 15° C. for 0.5 hour. To the reaction mixture was added 4-azaspiro [2.5]octan-7-one (1.43 g, 8.85 mmol, 1 eq, HCl) at 15° C. and the solution was stirred at 15° C. for 0.5 hour. LCMS showed (1E)-2, 6-dichloro-N-(p-tolylsulfonyl) benzohydrazonoyl chloride was consumed completely and desired mass was detected. The reaction mixture was concentrated to give desired N—[(E)-[(2,6-dichlorophenyl)-(7-oxo-4-azaspiro[2.5]octan-4-yl)methylene]amino]-4-methyl-benzenesulfonamide (4 g, crude) as a yellow solid. MS (ESI): mass calcd. For C₂₁H₂₁N₃Cl₂SO₃ 465.07, m/z found 466.1 [M+H]⁺.

Step 3: The mixture of N—[(E)-[(2,6-dichlorophenyl)-(7-oxo-4-azaspiro[2.5]octan-4-yl)methylene]amino]-4-methyl-benzenesulfonamide (4 g, 8.58 mmol, 1 eq) and K₂CO₃ (11.8 g, 85.8 mmol, 10 eq) in DMF (40 mL) was stirred at 100° C. for 12 hours. LCMS showed N—[(E)-[(2,6-dichlorophenyl)-(7-oxo-4-azaspiro[2.5]octan-4-yl)methylene]amino]-4-methyl-benzenesulfonamide was consumed completely and desired mass was detected. The reaction was poured into water (30 mL) and extracted with MTBE (2*50 mL). The combined organics were concentrated to get a residue. The residue was purified by flash silica gel chromatography (ISCO®; 40 g SepaFlash® Silica Flash Column, Eluent of 0˜50% ethyl acetate/petroleum ether gradient @80 mL/min) to give desired 4-[4-chloro-1-(p-tolylsulfonyl) indazol-3-yl]-4-azaspiro [2.5]octan-7-one (1.0 g, 2.33 mmol, 27.1% yield) as a white solid. MS (ESI): mass calcd. For C₂₁H₂₀N₃ClSO₃ 429.09, m/z found 432.1 [M+H]⁺.

### Intermediate-L10: 2-(3, 3-dimethylpiperazin-1-yl)-5-fluoro-pyrimidine

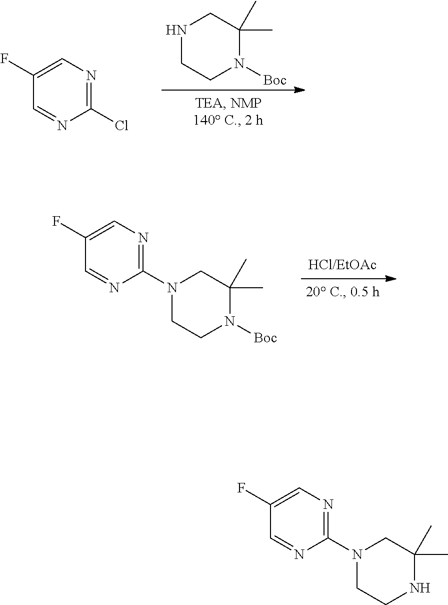 [SMILES: Fc1cnc(Cl)nc1 ; CC(C)(C)OC(=O)N1CCNCC1(C)C ; CC(C)(C)OC(=O)N1CCN(c2ncc(F)cn2)CC1(C)C ; CC1(C)CN(c2ncc(F)cn2)CCN1]

Step 1: To a solution of 2-chloro-5-fluoro-pyrimidine (618 mg, 4.67 mmol, 578 μL, 1 eq) in NMP (10 mL) was added TEA (1.42 g, 14.0 mmol, 1.95 mL, 3 eq) and tert-butyl 2, 2-dimethylpiperazine-1-carboxylate (1 g, 4.67 mmol, 1 eq). The mixture was stirred at 140° C. for 2 hours. LC-MS showed 2-chloro-5-fluoro-pyrimidine was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by column chromatography (SiO₂, petroleum ether/Ethyl acetate=1/0 to 0/1) to give desired tert-butyl 4-(5-fluoropyrimidin-2-yl)-2, 2-dimethyl-piperazine-1-carboxylate (1.4 g, crude) as a colorless oil. MS (ESI): mass calcd. For C₁₅H₂₃FN₄O₂ 310.18, m/z found 255.1[M+H−56]⁺.

Step 2: A mixture of tert-butyl 4-(5-fluoropyrimidin-2-yl)-2, 2-dimethyl-piperazine-1-carboxylate (1.4 g, 4.51 mmol, 1 eq) in HCl/EtOAc (4 M, 4 mL) was stirred at 20° C. for 0.5 hour. LC-MS showed tert-butyl 4-(5-fluoropyrimidin-2-yl)-2, 2-dimethyl-piperazine-1-carboxylate was consumed completely and desired mass was detected. The reaction mixture was concentrated to give desired 2-(3, 3-dimethylpiperazin-1-yl)-5-fluoro-pyrimidine (1.3 g, crude, HCl) as a white solid. MS (ESI): mass calcd. For C₁₀H₁₅FN₄ 210.13, m/z found 211.1[M+H]⁺.

### Intermediate-L11: 4-chloro-3-[4-(5-fluoropyrimidin-2-yl)-2, 2-dimethyl-piperazin-1-yl]-1H-indazole

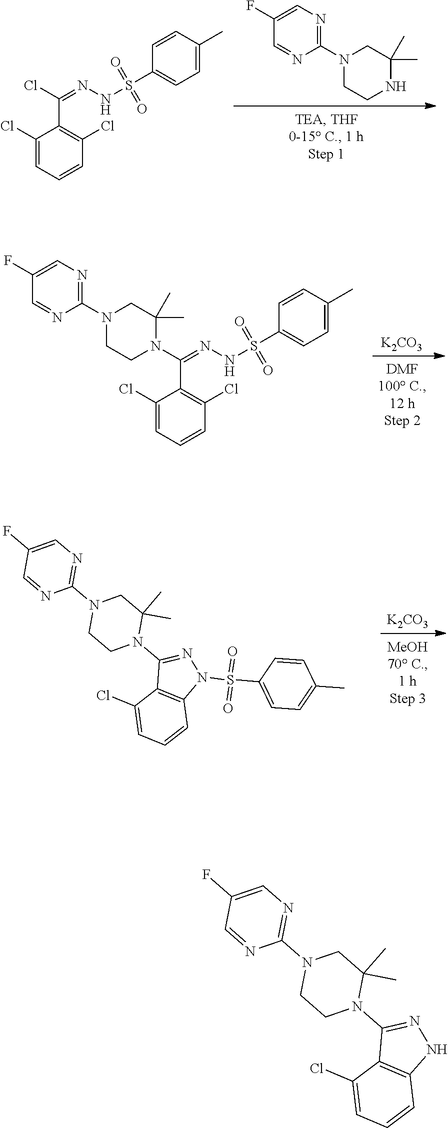

Step 1: To the solution of (1E)-2,6-dichloro-N-(p-tolylsulfonyl)benzohydrazonoyl chloride (459 mg, 1.22 mmol, 1 eq) in THF (3 mL) was added TEA (1.23 g, 12.2 mmol, 1.69 mL, 10 eq) at 15° C. and the solution was stirred at 15° C. for 0.5 hour. To the reaction mixture was added 2-(3,3-dimethylpiperazin-1-yl)-5-fluoro-pyrimidine (300 mg, 1.22 mmol, 1 eq, HCl) at 0° C. and the solution was stirred at 15° C. for 0.5 hour. LCMS showed 2-(3, 3-dimethylpiperazin-1-yl)-5-fluoro-pyrimidine was consumed completely and desired mass was detected. The reaction mixture was concentrated to give desired N—[(E)-[(2, 6-dichlorophenyl)-[4-(5-fluoropyrimidin-2-yl)-2, 2-dimethyl-piperazin-1-yl]methylene]amino]-4-methyl-benzenesulfonamide (1.8 g, crude) as a yellow solid. MS (ESI): mass calcd. For C₂₁H₂₃C₁₂N₃O₂S 451.09, m/z found 452.1[M+H]⁺.

Step 2: To a solution of N—[(E)-[(2, 6-dichlorophenyl)-[4-(5-fluoropyrimidin-2-yl)-2, 2-dimethyl-piperazin-1-yl]methylene]amino]-4-methyl-benzenesulfonamide (1.8 g, 3.26 mmol, 1 eq) in DMF (10 mL) was added K₂CO₃ (1.80 g, 13.1 mmol, 4 eq). The mixture was stirred at 100° C. for 12 hours. LC-MS showed N—[(E)-[(2, 6-dichlorophenyl)-[4-(5-fluoropyrimidin-2-yl)-2, 2-dimethyl-piperazin-1-yl]methylene]amino]-4-methyl-benzenesulfonamide was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give desired 4-chloro-3-[4-(5-fluoropyrimidin-2-yl)-2, 2-dimethyl-piperazin-1-yl]-1-(p-tolylsulfonyl) indazole (1.8 g, crude) as an orange oil. MS (ESI): mass calcd. For C₂₄H₂₄ClFN₆O₂S 514.14, m/z found 515.2[M+H]⁺.

Step 3: To a solution of 4-chloro-3-[4-(5-fluoropyrimidin-2-yl)-2, 2-dimethyl-piperazin-1-yl]-1-(p-tolylsulfonyl) indazole (1.8 g, 3.50 mmol, 1 eq) in MeOH (10 mL) was added K₂CO₃ (2.42 g, 17.5 mmol, 5 eq). The mixture was stirred at 70° C. for 1 hour. LC-MS showed 4-chloro-3-[4-(5-fluoropyrimidin-2-yl)-2, 2-dimethyl-piperazin-1-yl]-1-(p-tolylsulfonyl) indazole was consumed completely and desired compound was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by column chromatography (SiO₂, petroleum ether/Ethyl acetate=1/0 to 1/1) to give desired 4-chloro-3-[4-(5-fluoropyrimidin-2-yl)-2, 2-dimethyl-piperazin-1-yl]-1H-indazole (350 mg, crude) as a yellow oil. MS (ESI): mass calcd. For C₁₇H₁₈ClFN₆ 360.13, m/z found 361.1[M+H]⁺.

### Intermediate-L12: 7-(5-fluoro-2-pyridyl)-4, 7-diazaspiro [2.5]octane

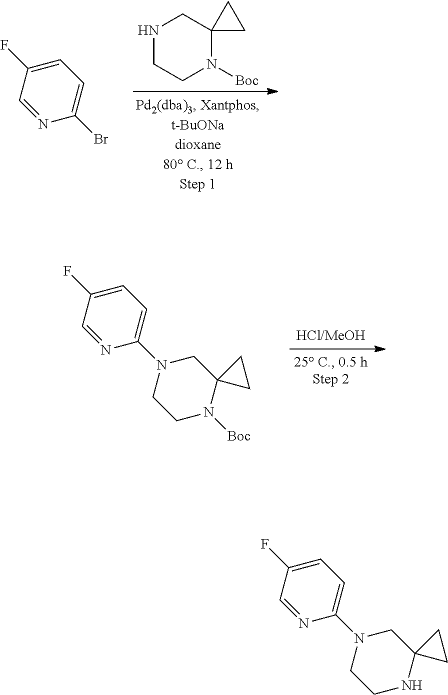 [SMILES: Fc1ccc(Br)nc1 ; CC(C)(C)OC(=O)N1CCNCC12CC2 ; CC(C)(C)OC(=O)N1CCN(c2ccc(F)cn2)CC12CC2 ; Fc1ccc(N2CCNC3(CC3)C2)nc1]

Step 1: A mixture of 2-bromo-5-fluoro-pyridine (1 g, 5.68 mmol, 1 eq), tert-butyl 4, 7-diazaspiro [2.5]octane-4-carboxylate (1.21 g, 5.68 mmol, 1 eq), Pd₂(dba)₃ (312 mg, 341 μmol, 0.06 eq), Xantphos (395 mg, 682 μmol, 0.12 eq) and t-BuONa (1.64 g, 17.0 mmol, 3 eq) in dioxane (10 mL) was degassed and purged with N₂ for 3 times, and then the mixture was stirred at 80° C. for 12 hours under the atmosphere of nitrogen. LC-MS showed 2-bromo-5-fluoro-pyridine was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by flash silica gel chromatography (ISCO®; 40 g SepaFlash® Silica Flash Column, Eluent of 0˜50% EtOAc/petroleum ether gradient @80 mL/min) to give desired tert-butyl 7-(5-fluoro-2-pyridyl)-4, 7-diazaspiro [2.5]octane-4-carboxylate (1.5 g, crude) was obtained as a yellow oil. MS (ESI): mass calcd. For C₁₆H₂₂FN₃O₂ 307.17, m/z found 308.1 [M+H]⁺.

Step 2: A mixture of tert-butyl 7-(5-fluoro-2-pyridyl)-4, 7-diazaspiro [2.5]octane-4-carboxylate (1.50 g, 4.88 mmol, 1 eq) in HCl/MeOH (4 M, 10 mL) was stirred at 25° C. for 0.5 hour. LC-MS showed tert-butyl 7-(5-fluoro-2-pyridyl)-4, 7-diazaspiro [2.5]octane-4-carboxylate was consumed completely and desired mass was detected. The reaction mixture was concentrated to give desired 7-(5-fluoro-2-pyridyl)-4, 7-diazaspiro [2.5]octane (1.1 g, crude) as a white solid. MS (ESI): mass calcd. For C₁₁H₁₄FN₃ 207.12, m/z found 208.1 [M+H]⁺.

### Intermediate-L13: 4, 6-difluoro-3-(7-(5-fluoropyridin-2-yl)-4, 7-diazaspiro [2.5]octan-4-yl)-1H-indazole

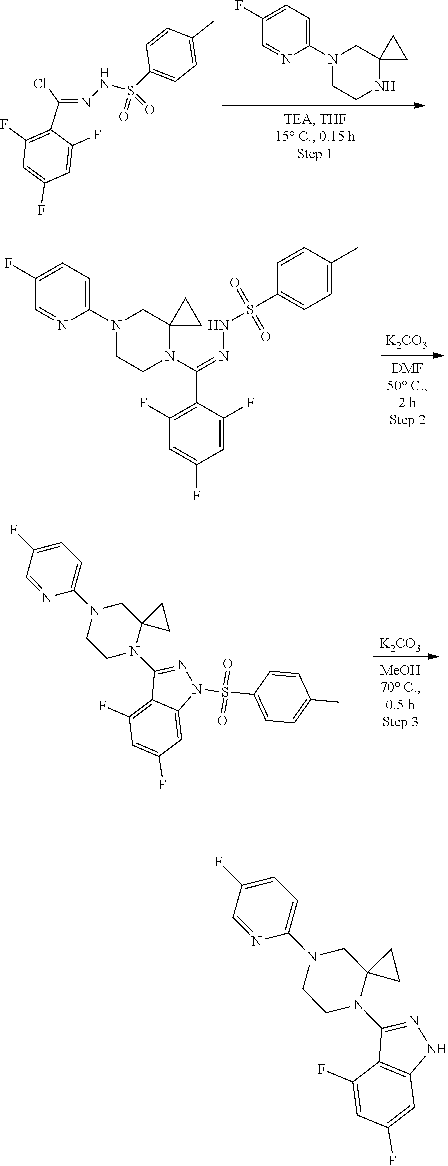

Step 1: To a solution of (1Z)-2,4,6-trifluoro-N-(p-tolylsulfonyl)benzohydrazonoyl chloride (357 mg, 985 μmol, 1.2 eq) in THF (5 mL) was added TEA (208 mg, 2.05 mmol, 286 μL, 2.5 eq) and 7-(5-fluoro-2-pyridyl)-4, 7-diazaspiro [2.5]octane (200 mg, 821 μmol, 1 eq, HCl). The mixture was stirred at 15° C. for 0.15 hour. LC-MS showed (1Z)-2, 4, 6-trifluoro-N-(p-tolylsulfonyl) benzohydrazonoyl chloride was consumed completely and desired mass was detected. The crude was added H₂O (10 mL), and extracted with EtOAc (45 mL). The combined organic layers were washed with brine (10 mL), dried over Na₂SO₄, filtered and concentrated under reduced pressure to give desired (Z)—N′-((7-(5-fluoropyridin-2-yl)-4, 7-diazaspiro [2.5]octan-4-yl) (2, 4, 6-trifluorophenyl) methylene)-4-methylbenzenesulfonohydrazide (500 mg, crude) as a yellow solid. MS (ESI): mass calcd. For C₂₅H₂₃F₄N₅O₂S 533.15, m/z found 534.2 [M+H]⁺.

Step 2: A mixture of N—[(Z)—[[7-(5-fluoro-2-pyridyl)-4, 7-diazaspiro [2.5]octan-4-yl]-(2, 4, 6-trifluorophenyl) methylene]amino]-4-methyl-benzenesulfonamide (484 mg, 907 μmol, 1 eq), K₂CO₃ (1.25 g, 9.07 mmol, 10 eq) in DMF (10 mL) was degassed and purged with N₂ for 3 times, and then the mixture was stirred at 50° C. for 2 hours under the atmosphere of nitrogen. LC-MS showed N—[(Z)—[[7-(5-fluoro-2-pyridyl)-4, 7-diazaspiro [2.5]octan-4-yl]-(2, 4, 6-trifluorophenyl) methylene]amino]-4-methyl-benzenesulfonamide was consumed completely and desired mass was detected. The crude was added H₂O (10 mL), and extracted with EtOAc (45 mL). The combined organic layers were washed with brine (10 mL), dried over Na₂SO₄, filtered and concentrated under reduced pressure to give desired 4, 6-difluoro-3-(7-(5-fluoropyridin-2-yl)-4, 7-diazaspiro [2.5]octan-4-yl)-1-tosyl-1H-indazole (460 mg, crude) as a brown oil. MS (ESI): mass calcd. For C₂₅H₂₂F₃N₅O₂S 513.14, m/z found 514.0 [M+H]⁺.

Step 3: To a solution of 4, 6-difluoro-3-[7-(5-fluoro-2-pyridyl)-4, 7-diazaspiro [2.5]octan-4-yl]-1-(p-tolylsulfonyl) indazole (460 mg, 896 μmol, 1 eq) in MeOH (10 mL) was added K₂CO₃ (619 mg, 4.48 mmol, 5 eq). The mixture was stirred at 75° C. for 0.5 hour. LC-MS showed 4, 6-difluoro-3-[7-(5-fluoro-2-pyridyl)-4, 7-diazaspiro [2.5]octan-4-yl]-1-(p-tolylsulfonyl) indazole was consumed completely and desired mass was detected. The reaction mixture was concentrated under reduced pressure to give a residue. The crude was added H₂O (10 mL), and extracted with EtOAc (45 mL). The combined organic layers were washed with brine (10 mL), dried over Na₂SO₄, filtered and concentrated under reduced pressure to give a residue. The residue was purified by column chromatography (SiO₂, petroleum ether/Ethyl acetate=5/1 to 4/1) to give desired 4, 6-difluoro-3-(7-(5-fluoropyridin-2-yl)-4, 7-diazaspiro [2.5]octan-4-yl)-1H-indazole (140 mg, 390 μmol, 43.5% yield) as a brown solid. MS (ESI): mass calcd. For C₁₈H₁₆F₃N₅ 359.14, m/z found 360.1 [M+H]⁺.

### Intermediate-L14: 4, 6-difluoro-3-(7-(5-fluoropyridin-2-yl)-4, 7-diazaspiro [2.5]octan-4-yl)-1H-indazole

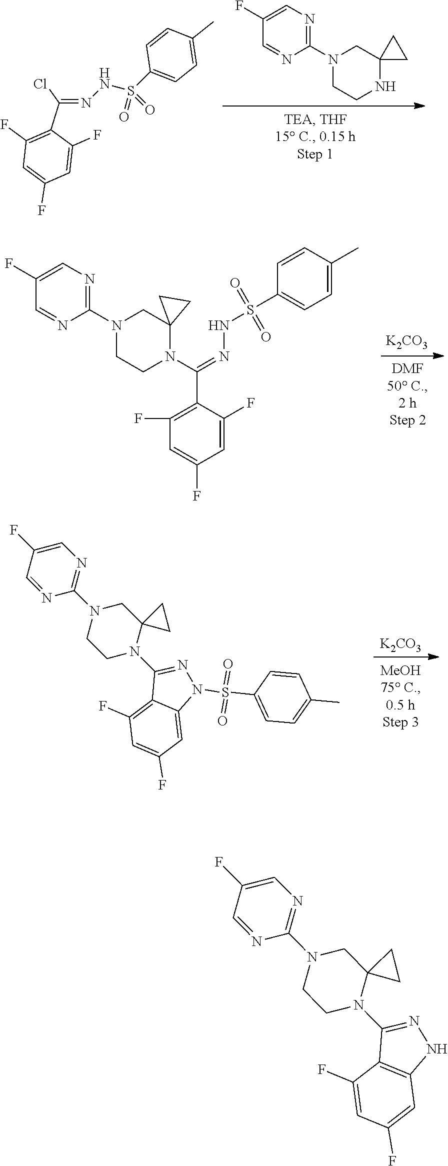

Step 1: To a solution of (1Z)-2,4,6-trifluoro-N-(p-tolylsulfonyl)benzohydrazonoyl chloride (357 mg, 985 μmol, 1.2 eq) in THF (5 mL) was added TEA (208 mg, 2.05 mmol, 286 μL, 2.5 eq) and 7-(5-fluoropyrimidin-2-yl)-4, 7-diazaspiro [2.5]octane (200 mg, 821 μmol, 1 eq, HCl). The mixture was stirred at 15° C. for 0.15 hour. LC-MS showed (1Z)-2, 4, 6-trifluoro-N-(p-tolylsulfonyl) benzohydrazonoyl chloride was consumed completely and desired mass was detected. The crude was added H₂O (10 mL), and extracted with EtOAc (45 mL). The combined organic layers were washed with brine (10 mL), dried over Na₂SO₄, filtered and concentrated under reduced pressure to give desired (Z)—N′-((7-(5-fluoropyrimidin-2-yl)-4,7-diazaspiro [2.5]octan-4-yl) (2,4,6-trifluorophenyl) methylene)-4-methylbenzenesulfonohydrazide (500 mg, crude) as a yellow solid. MS (ESI): mass calcd. For C₂₄H₂₂F₄N₆O₂S 534.15, m/z found 535.2 [M+H]⁺.

Step 2: A mixture of N—[(Z)—[[7-(5-fluoro-2-pyridyl)-4, 7-diazaspiro [2.5]octan-4-yl]-(2, 4, 6-trifluorophenyl) methylene]amino]-4-methyl-benzenesulfonamide (484 mg, 907 μmol, 1 eq), K₂CO₃ (1.25 g, 9.07 mmol, 10 eq) in DMF (10 mL) was degassed and purged with N₂ for 3 times, and then the mixture was stirred at 50° C. for 2 hours under the atmosphere of nitrogen. LC-MS showed N—[(Z)—[[7-(5-fluoro-2-pyridyl)-4, 7-diazaspiro [2.5]octan-4-yl]-(2, 4, 6-trifluorophenyl) methylene]amino]-4-methyl-benzenesulfonamide was consumed completely and desired mass was detected. The crude was added H₂O (10 mL), and extracted with EtOAc 45 (mL). The combined organic layers were washed with brine (10 mL), dried over Na₂SO₄, filtered and concentrated under reduced pressure to give desired 4, 6-difluoro-3-(7-(5-fluoropyridin-2-yl)-4, 7-diazaspiro [2.5]octan-4-yl)-1-tosyl-1H-indazole (460 mg, crude) as a brown oil. MS (ESI): mass calcd. For C₂₅H₂₂F₃N₅O₂S 513.14, m/z found 514.0 [M+H]⁺.

Step 3: To a solution of 4, 6-difluoro-3-[7-(5-fluoro-2-pyridyl)-4, 7-diazaspiro [2.5]octan-4-yl]-1-(p-tolylsulfonyl) indazole (460 mg, 896 μmol, 1 eq) in MeOH (10 mL) was added K₂CO₃ (619 mg, 4.48 mmol, 5 eq). The mixture was stirred at 75° C. for 0.5 hour. LC-MS showed 4, 6-difluoro-3-[7-(5-fluoro-2-pyridyl)-4, 7-diazaspiro [2.5]octan-4-yl]-1-(p-tolylsulfonyl) indazole was consumed completely and desired mass was detected. The reaction mixture was concentrated under reduced pressure to give a residue. The crude was added H₂O (10 mL), and extracted with EtOAc (45 mL). The combined organic layers were washed with brine (10 mL), dried over Na₂SO₄, filtered and concentrated under reduced pressure to give a residue. The residue was purified by column chromatography (SiO₂, petroleum ether/EtOAc=5/1 to 4/1) to give desired 4, 6-difluoro-3-(7-(5-fluoropyridin-2-yl)-4, 7-diazaspiro [2.5]octan-4-yl)-1H-indazole (140 mg, 390 μmol, 43.5% yield) as a brown solid. MS (ESI): mass calcd. For C₁₈H₁₆F₃N₅ 359.14, m/z found 360.1 [M+H]⁺.

### Intermediate-L15: 4-chloro-3-(4, 7-diazaspiro [2.5]octan-4-yl)-1-[(5-isopropylsulfonyl-2-thienyl) sulfonyl]indazole

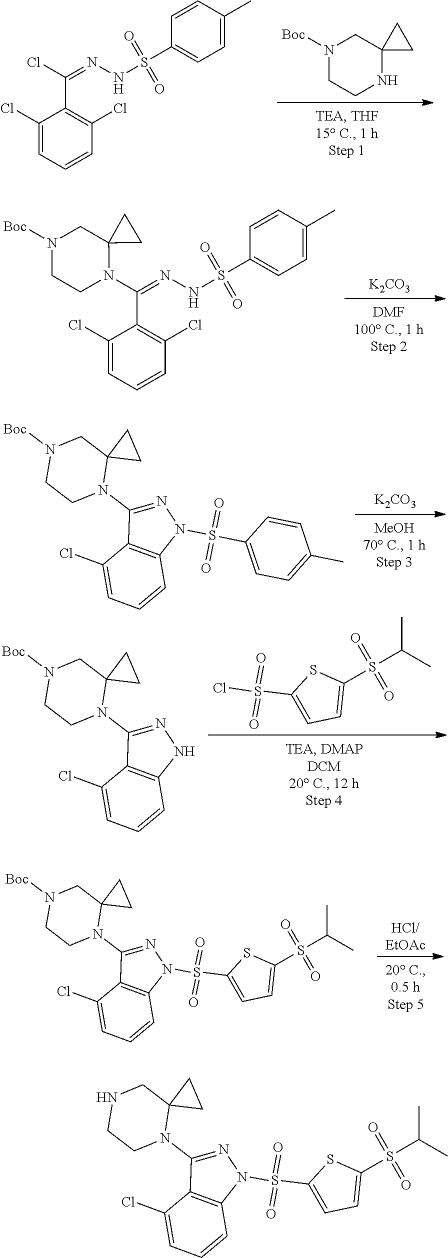

Step 1: To the solution of (1E)-2,6-dichloro-N-(p-tolylsulfonyl)benzohydrazonoyl chloride (890 mg, 2.36 mmol, 1 eq) in THF (5 mL) was added TEA (2.38 g, 23.6 mmol, 3.28 mL, 10 eq) at 15° C. and the solution was stirred at 15° C. for 0.5 hour. To the reaction mixture was added tert-butyl 4, 7-diazaspiro [2.5]octane-7-carboxylate (500 mg, 2.36 mmol, 1 eq) at 15° C. and the solution was stirred at 15° C. for 0.5 hour. LCMS showed (1Z)-2, 6-dichloro-4-fluoro-N-(p-tolylsulfonyl) benzohydrazonoyl chloride was consumed completely and desired mass was detected. The reaction mixture was concentrated to give desired tert-butyl 4-[(E)-C-(2, 6-dichlorophenyl)-N-(p-tolylsulfonylamino) carbonimidoyl]-4, 7-diazaspiro [2.5]octane-7-carboxylate (3 g, crude) as a yellow solid. MS (ESI): mass calcd. For C₂₅H₃₀C₁₂N₄O₄S 552.14, m/z found 553.3[M+H]⁺.

Step 2: To a solution of tert-butyl 4-[(E)-C-(2, 6-dichlorophenyl)-N-(p-tolylsulfonylamino) carbonimidoyl]-4, 7-diazaspiro [2.5]octane-7-carboxylate (3 g, 5.42 mmol, 1 eq) in DMF (10 mL) was added K₂CO₃ (3.00 g, 21.7 mmol, 4 eq). The mixture was stirred at 100° C. for 12 hours. LC-MS showed tert-butyl 4-[(E)-C-(2, 6-dichlorophenyl)-N-(p-tolylsulfonylamino) carbonimidoyl]-4, 7-diazaspiro [2.5]octane-7-carboxylate was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give desired tert-butyl 4-[4-chloro-1-(p-tolylsulfonyl)indazol-3-yl]-4, 7-diazaspiro [2.5]octane-7-carboxylate (2.8 g, crude) as an orange oil. MS (ESI): mass calcd. For C₂₅H₂₉ClN₄O₄S 516.16, m/z found 461.1[M+H−56]⁺.

Step 3: To a solution of tert-butyl 4-[4-chloro-1-(p-tolylsulfonyl) indazol-3-yl]-4, 7-diazaspiro [2.5]octane-7-carboxylate (2.8 g, 5.42 mmol, 1 eq) in MeOH (10 mL) was added K₂CO₃ (3.74 g, 27.1 mmol, 5 eq). The mixture was stirred at 70° C. for 1 hour. LC-MS showed tert-butyl 4-[4-chloro-1-(p-tolylsulfonyl) indazol-3-yl]-4, 7-diazaspiro [2.5]octane-7-carboxylate was consumed completely and desired compound was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by column chromatography (SiO₂, petroleum ether/Ethyl acetate=1/0 to 1/1) to give desired tert-butyl 4-(4-chloro-1H-indazol-3-yl)-4, 7-diazaspiro [2.5]octane-7-carboxylate (550 mg, crude) as a yellow oil. MS (ESI): mass calcd. For C₁₈H₂₃ClN₄O₂ 362.15, m/z found 363.1[M+H]⁺.

Step 4: To a solution of 5-isopropylsulfonylthiophene-2-sulfonyl chloride (119 mg, 413 μmol, 1.5 eq) and tert-butyl 4-(4-chloro-1H-indazol-3-yl)-4,7-diazaspiro[2.5]octane-7-carboxylate (100 mg, 276 μmol, 1 eq) in DCM (2 mL) was added TEA (55.8 mg, 551 μmol, 76.7 μL, 2 eq) and DMAP (3.37 mg, 27.6 μmol, 0.1 eq) at 0° C. The mixture was stirred at 20° C. for 0.5 hour. LC-MS showed tert-butyl 4-(4-chloro-1H-indazol-3-yl)-4, 7-diazaspiro [2.5]octane-7-carboxylate was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (SiO₂, petroleum ether/Ethyl acetate=2/1) to give desired tert-butyl 4-[4-chloro-1-[(5-isopropylsulfonyl-2-thienyl) sulfonyl]indazol-3-yl]-4, 7-diazaspiro [2.5]octane-7-carboxylate (160 mg, crude) as a white solid. MS (ESI): mass calcd. For C₂₅H₃₁ClN₄O₆S₃ 614.11, m/z found 559.1[M+H−56]⁺.

Step 5: The mixture of tert-butyl 4-[4-chloro-1-[(5-isopropylsulfonyl-2-thienyl)sulfonyl]indazol-3-yl]-4,7-diazaspiro[2.5]octane-7-carboxylate (160 mg, 260 μmol, 1 eq) in HCl/EtOAc (4 M, 2 mL) was stirred at 20° C. for 0.5 hour. LC-MS showed tert-butyl 4-[4-chloro-1-[(5-isopropylsulfonyl-2-thienyl) sulfonyl]indazol-3-yl]-4, 7-diazaspiro [2.5]octane-7-carboxylate was consumed completely and desired mass was detected. The reaction mixture was concentrated to give desired 4-chloro-3-(4, 7-diazaspiro [2.5]octan-4-yl)-1-[(5-isopropylsulfonyl-2-thienyl) sulfonyl]indazole (130 mg, crude, HCl) as a yellow solid. MS (ESI): mass calcd. For C₂₀H₂₃ClN₄O₄S₃ 514.06, m/z found 515.0[M+H]⁺.

### Intermediate-L16: (E)-2, 6-difluoro-N′-tosylbenzohydrazonoyl chloride

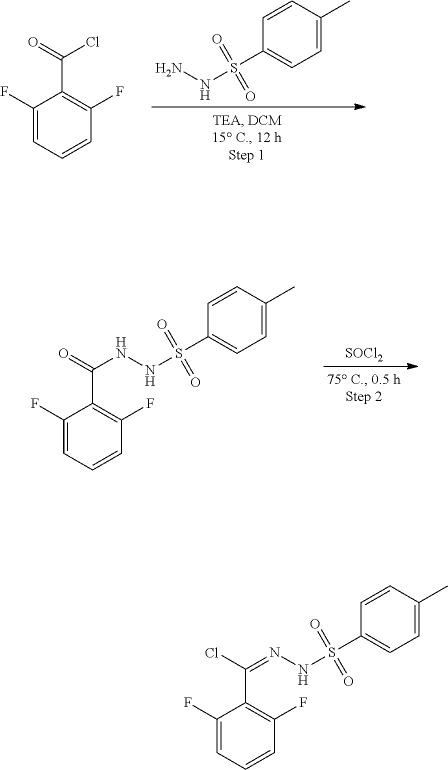 [SMILES: O=C(Cl)c1c(F)cccc1F ; Cc1ccc(S(=O)(=O)NN)cc1 ; Cc1ccc(S(=O)(=O)NNC(=O)c2c(F)cccc2F)cc1 ; "SOCl2" ; Cc1ccc(S(=O)(=O)N/N=C(/Cl)c2c(F)cccc2F)cc1]

Step 1: To a solution of 2, 6-difluorobenzoyl chloride (5 g, 28.3 mmol, 3.57 mL, 1 eq) in DCM (50 mL) was added TEA (7.16 g, 70.8 mmol, 9.85 mL, 2.5 eq) and 4-methylbenzenesulfonohydrazide (5.80 g, 31.2 mmol, 1.1 eq). The mixture was stirred at 15° C. for 12 hours. LC-MS showed 2, 6-difluorobenzoyl chloride was consumed completely and one main peak with desired mass was detected. The crude was added HCl (1M, 100 mL), the reaction mixture was concentrated under reduced pressure to give desired N′-(2, 6-difluorobenzoyl)-4-methylbenzenesulfonohydrazide (5.0 g, crude) as a white solid. MS (ESI): mass calcd. For C₁₄H₁₂F₂N₂O₃S 326.05, m/z found 327.2 [M+H]⁺.

Step 2: To a solution of 2, 6-difluoro-N′-(p-tolylsulfonyl) benzohydrazide (700 mg, 2.15 mmol, 1 eq) in SOCl₂ (10 mL). The mixture was stirred at 75° C. for 0.5 hour. TLC indicated 2, 6-difluoro-N′-(p-tolylsulfonyl) benzohydrazide was consumed completely and one new spot formed. The reaction mixture was concentrated under reduced pressure to give desired (E)-2, 6-difluoro-N′-tosylbenzohydrazonoyl chloride (700 mg, crude) as a light yellow solid.

### Intermediate-L17: 7-(3, 5-difluoro-2-pyridyl)-4, 7-diazaspiro [2.5]octane

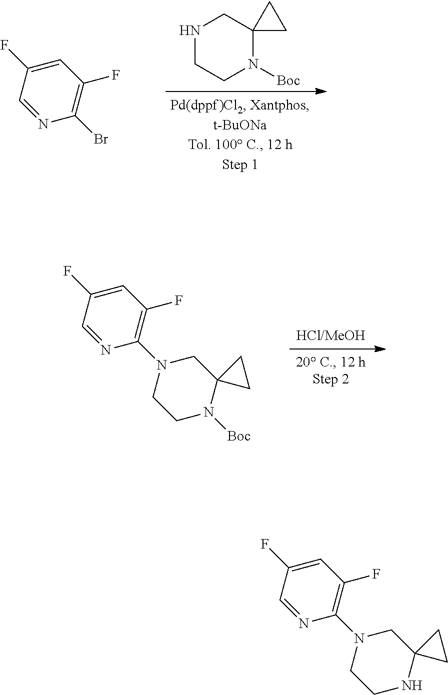 [SMILES: Fc1cnc(Br)c(F)c1 ; CC(C)(C)OC(=O)N1CCNCC12CC2 ; CC(C)(C)OC(=O)N1CCN(c2ncc(F)cc2F)CC12CC2 ; Fc1cnc(N2CCNC3(CC3)C2)c(F)c1]

Step 1: A mixture of 2-bromo-3, 5-difluoro-pyridine (350 mg, 1.80 mmol, 1 eq), tert-butyl 4, 7-diazaspiro [2.5]octane-4-carboxylate (421 mg, 1.98 mmol, 1.1 eq), Pd(dppf)C₁₂ (132 mg, 180 μmol, 0.1 eq), Xantphos (209 mg, 361 μmol, 0.2 eq) and t-BuONa (347 mg, 3.61 mmol, 2 eq) in Tol. (3 mL) was degassed and purged with N₂ for 3 times, and then the mixture was stirred at 100° C. for 12 hours under the atmosphere of nitrogen. LC-MS showed 2-bromo-3, 5-difluoro-pyridine was consumed completely and several new peaks were shown on LC-MS and ˜30% of mass of desired compound was detected. The reaction mixture was concentrated under reduced pressure to remove Tol. The residue was diluted with H₂O (10 mL) and extracted with EtOAc (10 mL*2). The combined organic layers were washed with brine (5 mL*2), dried over anhydrous Na₂SO₄, filtered and concentrated under reduced pressure to give a residue. The residue was purified by prep-TLC (SiO₂, petroleum ether/EtOAc=5/1) to give desired tert-butyl 7-(3, 5-difluoro-2-pyridyl)-4, 7-diazaspiro [2.5]octane-4-carboxylate (380 mg, 1.17 mmol, 65.5% yield) as a brown oil. MS (ESI): mass calcd. For C₁₆H₂₁N₃F₂O₂ 325.16, m/z found 326.0 [M+H]⁺.

Step 2: A mixture of tert-butyl 7-(3, 5-difluoro-2-pyridyl)-4, 7-diazaspiro [2.5]octane-4-carboxylate (380 mg, 1.17 mmol, 1 eq) in HCl/MeOH (4 M, 4 mL, 13.7 eq) was degassed and purged with N₂ for 3 times, and then the mixture was stirred at 20° C. for 12 hours under the atmosphere of nitrogen. LC-MS showed tert-butyl 7-(3, 5-difluoro-2-pyridyl)-4, 7-diazaspiro [2.5]octane-4-carboxylate was consumed completely and one main peak with desired mass was detected. The reaction mixture was concentrated in vacuum to give desired 7-(3, 5-difluoro-2-pyridyl)-4, 7-diazaspiro [2.5]octane (330 mg, crude, HCl) as a yellow oil. MS (ESI): mass calcd. For C₁₁H₁₃N₃F₂HCl 225.11, m/z found 226.2 [M+H]⁺.

### Intermediate-L18: 3-[7-(3, 5-difluoro-2-pyridyl)-4, 7-diazaspiro [2.5]octan-4-yl]-4, 7-difluoro-1H-indazole

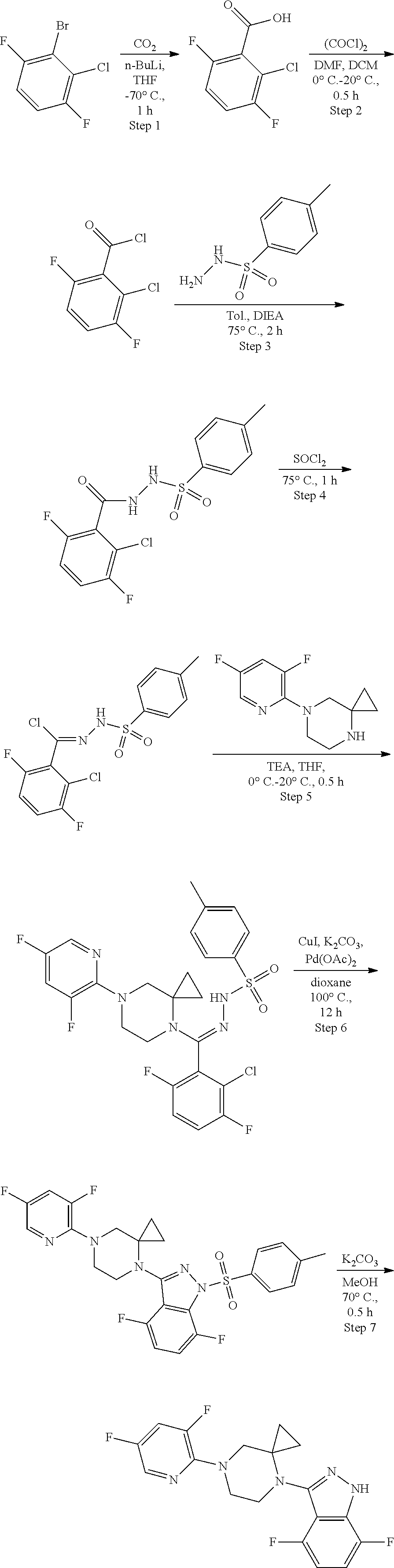

Step 1: To a solution of 2-bromo-3-chloro-1, 4-difluoro-benzene (1 g, 4.40 mmol, 1 eq) in THF (10 mL) was added dropwise n-BuLi (2.5 M, 2.64 mL, 1.5 eq) at −70° C. After addition, the mixture was stirred at this temperature for 0.5 hour, and then CO₂ (194 mg, 4.40 mmol, 1 eq) (15 psi) was added dropwise at −70° C. The resulting mixture was stirred at −70° C. for 0.5 hour. TLC (petroleum ether/EtOAc=3/1) indicated 2-bromo-3-chloro-1, 4-difluoro-benzene was consumed completely and one new spot formed. The residue was diluted with H₂O (100 mL) and extracted with EtOAc (50 mL*3). The combined organic layers were washed with brine (100 mL), dried over Na₂SO₄, filtered and concentrated under reduced pressure to give a residue. The residue was purified by prep-TLC (SiO₂, petroleum ether/EtOAc=3/1) to give desired 2-chloro-3, 6-difluoro-benzoic acid (0.3 g, 1.56 mmol, 40.0% yield) as a yellow solid.

Step 2: To a solution of 2-chloro-3, 6-difluoro-benzoic acid (0.3 g, 1.56 mmol, 1 eq) and DMF (11.4 mg, 156 μmol, 0.1 eq) in DCM (3 mL) was added (COCl)₂ (396 mg, 3.12 mmol, 2 eq) at 0° C. The mixture was stirred at 20° C. for 0.5 hour. TLC (petroleum ether/EtOAc=3/1) indicated 2-chloro-3, 6-difluoro-benzoic acid was consumed completely and one new spot formed. The reaction mixture was concentrated in vacuum to give desired 2-chloro-3, 6-difluoro-benzoyl chloride (320 mg, crude) as a yellow solid.

Step 3: To a solution of 2-chloro-3, 6-difluoro-benzoyl chloride (320 mg, 1.52 mmol, 4.20 mL, 1 eq) and DIEA (235 mg, 1.82 mmol, 1.2 eq) in Tol. (3 mL) was added 4-methylbenzenesulfonohydrazide (311 mg, 1.67 mmol, 1.1 eq). The mixture was stirred at 75° C. for 2 hours. LC-MS showed 2-chloro-3, 6-difluoro-benzoyl chloride was consumed completely and one main peak with desired mass was detected. The reaction mixture was filtered, and the filter cake was concentrated under reduced pressure to give a residue. The residue was purified by prep-TLC (SiO₂, petroleum ether/EtOAc=3/1) to give desired 2-chloro-3, 6-difluoro-N′-(p-tolylsulfonyl) benzohydrazide (230 mg, 638 μmol, 42.0% yield) as a white solid. MS (ESI): mass calcd. For C₁₄H₁₁N₂O₃SClF₂ 360.01, m/z found 361.1 [M+H]⁺.

Step 4: A mixture of 2-chloro-3, 6-difluoro-N′-(p-tolylsulfonyl) benzohydrazide (150 mg, 416 μmol, 1 eq) in SOCl₂ (3.06 g, 25.7 mmol, 1.87 mL, 61.9 eq) was degassed and purged with N₂ for 3 times, and then the mixture was stirred at 75° C. for 1 hour under the atmosphere of nitrogen. LC-MS (The simple was quenched with piperidine) showed 2-chloro-3, 6-difluoro-N′-(p-tolylsulfonyl) benzohydrazide was consumed completely and one main peak with desired mass was detected. The reaction mixture was filtered, and the filter cake was concentrated under reduced pressure to give desired (1Z)-2-chloro-3, 6-difluoro-N-(p-tolylsulfonyl) benzohydrazonoyl chloride (160 mg, crude) as a yellow solid. MS (ESI): mass calcd. For C₁₄H₁₀N₂C₁₂SO₂F₂ 377.98, m/z found 428.2 [M+H+50]⁺.

Step 5: To a solution of 7-(3, 5-difluoro-2-pyridyl)-4, 7-diazaspiro [2.5]octane (110 mg, 422 μmol, 1 eq, HCl) in THF (2 mL) was added dropwise TEA (128 mg, 1.27 mmol, 3 eq) at 20° C. After addition, the mixture was stirred at this temperature for 5 minutes, and then (1Z)-2-chloro-3, 6-difluoro-N-(p-tolylsulfonyl) benzohydrazonoyl chloride (160 mg, 422 μmol, 1 eq) in THF (0.5 mL) was added dropwise at 0° C. The resulting mixture was stirred at 20° C. for 25 minutes. LC-MS showed 7-(3, 5-difluoro-2-pyridyl)-4, 7-diazaspiro [2.5]octane was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give desired N—[(Z)-[(2-chloro-3, 6-difluoro-phenyl)-[7-(3, 5-difluoro-2-pyridyl)-4, 7-diazaspiro [2.5]octan-4-yl]methylene]amino]-4-methyl-benzenesulfonamide (0.3 g, crude) as a yellow oil. MS (ESI): mass calcd. For C₂₅H₂₂N₅SO₂ClF₄ 567.11, m/z found 568.3 [M+H]⁺.

Step 6: A mixture of N—[(Z)-[(2-chloro-3, 6-difluoro-phenyl)-[7-(3, 5-difluoro-2-pyridyl)-4, 7-diazaspiro [2.5]octan-4-yl]methylene]amino]-4-methyl-benzenesulfonamide (0.3 g, 528 μmol, 1 eq), CuI (10.1 mg, 52.8 μmol, 0.1 eq), Pd(OAc)₂ (23.7 mg, 106 μmol, 0.2 eq) and K₂CO₃ (183 mg, 1.32 mmol, 2.5 eq) in dioxane (3 mL) was degassed and purged with N₂ for 3 times, and then the mixture was stirred at 100° C. for 12 hours under the atmosphere of nitrogen. LC-MS showed N—[(Z)-[(2-chloro-3, 6-difluoro-phenyl)-[7-(3, 5-difluoro-2-pyridyl)-4, 7-diazaspiro [2.5]octan-4-yl]methylene]amino]-4-methyl-benzenesulfonamide was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give desired 3-[7-(3, 5-difluoro-2-pyridyl)-4, 7-diazaspiro [2.5]octan-4-yl]-4, 7-difluoro-1-(p-tolylsulfonyl) indazole (0.3 g, crude) as a yellow solid. MS (ESI): mass calcd. For C₂₅H₂₁N₅SO₂F₄ 531.13, m/z found 532.3 [M+H]⁺.

Step 7: To a solution of 3-[7-(3, 5-difluoro-2-pyridyl)-4, 7-diazaspiro [2.5]octan-4-yl]-4, 7-difluoro-1-(p-tolylsulfonyl) indazole (0.3 g, 564 μmol, 1 eq) in MeOH (4 mL) was added K₂CO₃ (390 mg, 2.82 mmol, 5 eq) was stirred at 70° C. for 0.5 hour. LC-MS showed a little of 3-[7-(3, 5-difluoro-2-pyridyl)-4, 7-diazaspiro [2.5]octan-4-yl]-4, 7-difluoro-1-(p-tolylsulfonyl) indazole remained. Several new peaks were shown on LC-MS and desired compound was detected. Then it was separated between water (5 mL) and EtOAc (10 mL). The organic phase was separated, washed with brine (5 mL), dried over Na₂SO₄, filtered and concentrated under reduced pressure to give the crude product. The residue was purified by prep-TLC (SiO₂, petroleum ether/EtOAc=5/1) to give desired 3-[7-(3, 5-difluoro-2-pyridyl)-4, 7-diazaspiro [2.5]octan-4-yl]-4, 7-difluoro-1H-indazole (40 mg, 106 μmol, 18.8% yield) as a yellow solid. MS (ESI): mass calcd. For C₁₈H₁₅N₅F₄ 377.13, m/z found 378.1 [M+H]⁺.

### Intermediate-L19: 7-(5-chloro-3-fluoro-2-pyridyl)-4, 7-diazaspiro [2.5]octane

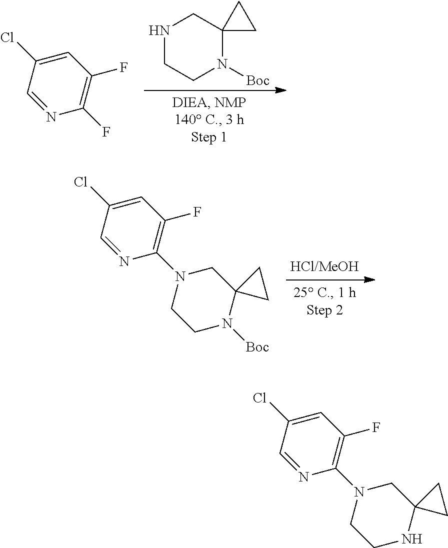

Step 1: To a solution of 5-chloro-2, 3-difluoro-pyridine (194 mg, 1.30 mmol, 1.1 eq) in NMP (3 mL) was added DIEA (152 mg, 1.18 mmol, 205 μL, 1 eq) and tert-butyl 4, 7-diazaspiro [2.5]octane-4-carboxylate (250 mg, 1.18 mmol, 1 eq). The mixture was stirred at 140° C. for 3 hours. LC-MS showed 5-chloro-2, 3-difluoro-pyridine was consumed completely and one main peak with desired m/z was detected. The reaction mixture was partitioned between EtOAc (3 mL) and H₂O (2 mL). The organic phase was separated, concentrated under reduced pressure to give a residue. The residue was purified by prep-TLC (SiO₂, petroleum ether/EtOAc=20/1) to give desired tert-butyl 7-(5-chloro-3-fluoro-2-pyridyl)-4, 7-diazaspiro [2.5]octane-4-carboxylate (330 mg, 965 μmol, 82.0% yield) as a colorless oil. MS (ESI): mass calcd. For C₁₆H₂₁ClFN₃O₂ 341.13, m/z found 242.0 [M+H−100]⁺.

Step 2: To a solution of tert-butyl 7-(5-chloro-3-fluoro-2-pyridyl)-4, 7-diazaspiro [2.5]octane-4-carboxylate (330 mg, 965 μmol, 1 eq) was added HCl/MeOH (4 M, 5 mL, 20.7 eq). The mixture was stirred at 25° C. for 1 hour. TLC (petroleum ether/Ethyl acetate=20:1) indicated tert-butyl 7-(5-chloro-3-fluoro-2-pyridyl)-4, 7-diazaspiro [2.5]octane-4-carboxylate was consumed completely and one new spot formed. The reaction mixture was concentrated under reduced pressure to remove MeOH to give desired 7-(5-chloro-3-fluoro-2-pyridyl)-4, 7-diazaspiro [2.5]octane (260 mg, crude, HCl) as a white solid.

### Intermediate-L20: 4-chloro-3-[7-(5-chloro-3-fluoro-2-pyridyl)-4, 7-diazaspiro [2.5]octan-4-yl]-1H-indazole

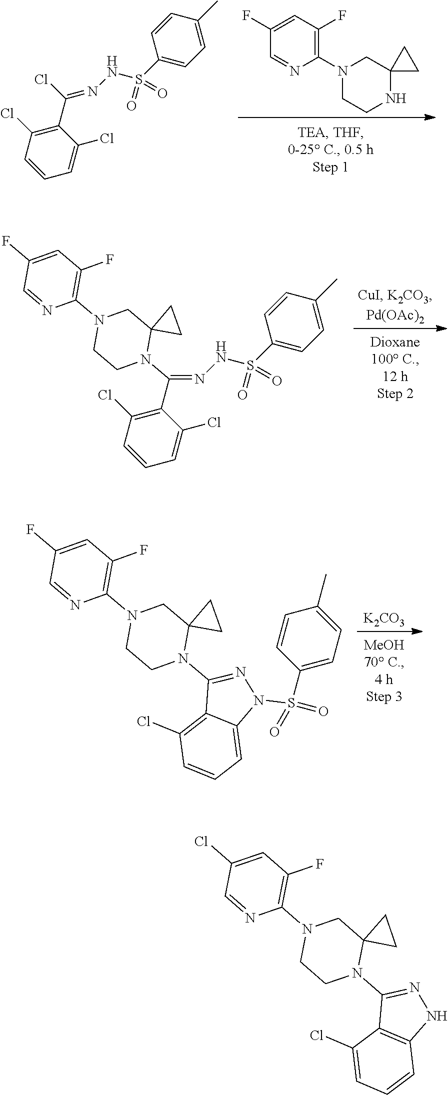

Step 1: To a solution of 7-(5-chloro-3-fluoro-2-pyridyl)-4,7-diazaspiro[2.5]octane (250 mg, 899 μmol, 1 eq, HCl) in THF (3 mL) was added dropwise TEA (182 mg, 1.80 mmol, 250 μL, 2 eq) at 25° C. After addition, the mixture was stirred 25° C. for 15 minutes, and then (1Z)-2,6-dichloro-N-(p-tolylsulfonyl)benzohydrazonoyl chloride (373 mg, 989 μmol, 1.1 eq) in THF (3 mL) was added dropwise at 0° C. The resulting mixture was stirred at 25° C. for 15 minutes. LC-MS showed 7-(5-chloro-3-fluoro-2-pyridyl)-4, 7-diazaspiro [2.5]octane was consumed completely and one main peak with desired m/z was detected. The reaction mixture was partitioned between EtOAc (8 mL) and H₂O (5 mL). The organic phase was separated, concentrated under reduced pressure to give desired N—[(Z)—[[7-(5-chloro-3-fluoro-2-pyridyl)-4, 7-diazaspiro [2.5]octan-4-yl]-(2, 6-dichlorophenyl) methylene]amino]-4-methyl-benzenesulfonamide (500 mg, crude) as a yellow solid.

Step 2: A mixture of N—[(Z)—[[7-(5-chloro-3-fluoro-2-pyridyl)-4, 7-diazaspiro [2.5]octan-4-yl]-(2, 6-dichlorophenyl) methylene]amino]-4-methyl-benzenesulfonamide (500 mg, 858 μmol, 1 eq), CuI (16.3 mg, 85.8 μmol, 0.1 eq), K₂CO₃ (296 mg, 2.14 mmol, 2.5 eq) and Pd(OAc)₂ (38.5 mg, 172 μmol, 0.2 eq) in dioxane (6 mL) was degassed and purged with N₂ for 3 times, and then the mixture was stirred at 100° C. for 12 hours under the atmosphere of nitrogen. TLC (petroleum ether/EtOAc=3/1) indicated N—[(Z)—[[7-(5-chloro-3-fluoro-2-pyridyl)-4, 7-diazaspiro [2.5]octan-4-yl]-(2, 6-dichlorophenyl) methylene]amino]-4-methyl-benzenesulfonamide was consumed completely and one new spot formed. The reaction mixture was partitioned between EtOAc (20 mL) and H₂O (10 mL). The organic phase was separated, dried over anhydrous Na₂SO₄, filtered and concentrated under reduced pressure to give desired 4-chloro-3-[7-(5-chloro-3-fluoro-2-pyridyl)-4, 7-diazaspiro [2.5]octan-4-yl]-1-(p-tolylsulfonyl)indazole (450 mg, crude) as a white solid.

Step 3: To a solution of 4-chloro-3-[7-(5-chloro-3-fluoro-2-pyridyl)-4, 7-diazaspiro [2.5]octan-4-yl]-1-(p-tolylsulfonyl) indazole (225 mg, 412 μmol, 1 eq) in MeOH (4 mL) was added K₂CO₃ (285 mg, 2.06 mmol, 5 eq). The mixture was stirred at 70° C. for 4 hours. LC-MS showed 4-chloro-3-[7-(5-chloro-3-fluoro-2-pyridyl)-4, 7-diazaspiro [2.5]octan-4-yl]-1-(p-tolylsulfonyl) indazole was consumed completely and one main peak with desired m/z was detected. The reaction mixture was concentrated under reduced pressure to remove MeOH. The residue was diluted with H₂O (10 mL) and extracted with DCM (30 mL). The combined organic layers were dried over anhydrous Na₂SO₄, filtered and concentrated under reduced pressure to give a residue. The residue was purified by prep-TLC (SiO₂, petroleum ether/EtOAc=3/1) to give desired 4-chloro-3-[7-(5-chloro-3-fluoro-2-pyridyl)-4, 7-diazaspiro [2.5]octan-4-yl]-1H-indazole (200 mg, 510 μmol, 61.9% yield) as a yellow oil. MS (ESI): mass calcd. For C₁₈H₁₆C₁₂FN₅ 391.08, m/z found 392.1 [M+H]⁺.

### Intermediate-L21: 3-[7-(5-chloro-3-fluoro-2-pyridyl)-4, 7-diazaspiro [2.5]octan-4-yl]-4, 7-difluoro-1H-indazole

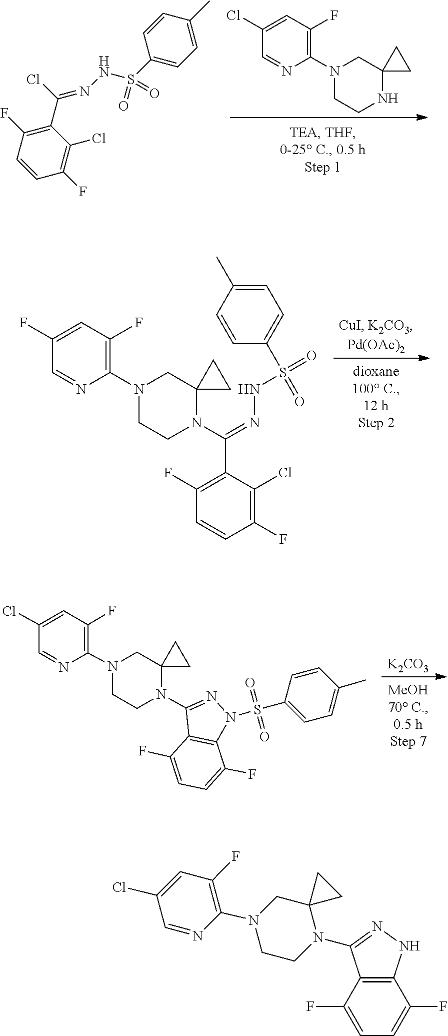

Step 1: To a solution of 7-(5-chloro-3-fluoro-2-pyridyl)-4, 7-diazaspiro [2.5]octane (403 mg, 1.45 mmol, 1 eq, HCl) in THF (10 mL) was added dropwise TEA (440 mg, 4.35 mmol, 606 μL, 3 eq) at 20° C. After addition, the mixture was stirred at this temperature for 5 minutes, and then (1Z)-2-chloro-3, 6-difluoro-N-(p-tolylsulfonyl) benzohydrazonoyl chloride (550 mg, 1.45 mmol, 1.0 eq) in THF (2 mL) was added dropwise at 0° C. The resulting mixture was stirred at 20° C. for 25 minutes. LC-MS showed (1Z)-2-chloro-3, 6-difluoro-N-(p-tolylsulfonyl) benzohydrazonoyl chloride was consumed completely and one main peak with desired mass was detected. The residue was diluted with H₂O (10 mL) and extracted with ethyl acetate (5 mL*3). The combined organic layers were washed with brine (10 mL), dried over Na₂SO₄, filtered and concentrated under reduced pressure to give desired N—[(Z)-[(2-chloro-3, 6-difluoro-phenyl)-[7-(5-chloro-3-fluoro-2-pyridyl)-4, 7-diazaspiro [2.5]octan-4-yl]methylene]amino]-4-methyl-benzenesulfonamide (0.9 g, crude) as a yellow oil. MS (ESI): mass calcd. For C₂₅H₂₂C₁₂F₃N₅O₂S 583.08, m/z found 584.3 [M+H]⁺.

Step 2: A mixture of N—[(Z)-[(2-chloro-3, 6-difluoro-phenyl)-[7-(5-chloro-3-fluoro-2-pyridyl)-4, 7-diazaspiro [2.5]octan-4-yl]methylene]amino]-4-methyl-benzenesulfonamide (0.9 g, 1.54 mmol, 1 eq), CuI (29.3 mg, 154 μmol, 0.1 eq), Pd(OAc)₂ (69.2 mg, 308 μmol, 0.2 eq) and K₂CO₃ (532 mg, 3.85 mmol, 2.5 eq) in dioxane (10 mL) was degassed and purged with N₂ for 3 times, and then the mixture was stirred at 100° C. for 12 hours under the atmosphere of nitrogen. LC-MS showed N—[(Z)-[(2-chloro-3, 6-difluoro-phenyl)-[7-(5-chloro-3-fluoro-2-pyridyl)-4, 7-diazaspiro [2.5]octan-4-yl]methylene]amino]-4-methyl-benzenesulfonamide was consumed completely and one main peak with desired mass was detected. The residue was diluted with H₂O (10 mL) and extracted with ethyl acetate (5 mL*3). The combined organic layers were washed with brine (10 mL), dried over Na₂SO₄, filtered and concentrated under reduced pressure to give desired 3-[7-(5-chloro-3-fluoro-2-pyridyl)-4, 7-diazaspiro [2.5]octan-4-yl]-4, 7-difluoro-1-(p-tolylsulfonyl) indazole (850 mg, crude) as a yellow solid. MS (ESI): mass calcd. For C₂₅H₂₁ClF₃N₅O₂S 547.11, m/z found 548.0 [M+H]⁺.

Step 3: To a solution of 3-[7-(5-chloro-3-fluoro-2-pyridyl)-4, 7-diazaspiro [2.5]octan-4-yl]-4, 7-difluoro-1-(p-tolylsulfonyl) indazole (0.85 g, 1.55 mmol, 1 eq) in MeOH (10 mL) was added K₂CO₃ (1.07 g, 7.76 mmol, 5 eq) was stirred at 70° C. for 0.5 hour. LC-MS showed a little of 3-[7-(5-chloro-3-fluoro-2-pyridyl)-4, 7-diazaspiro [2.5]octan-4-yl]-4, 7-difluoro-1-(p-tolylsulfonyl) indazole remained and desired mass was detected. Then it was separated between 5 mL of water and 10 mL of ethyl acetate. The organic phase was separated, washed with 5 mL of brine, dried over Na₂SO₄, filtered and concentrated under reduced pressure to give the crude product. The residue was purified by prep-TLC (SiO₂, petroleum ether/ethyl acetate=5/1) to give desired 3-[7-(5-chloro-3-fluoro-2-pyridyl)-4, 7-diazaspiro [2.5]octan-4-yl]-4, 7-difluoro-1H-indazole (0.1 g, 254 μmol, 16.4% yield) as a yellow solid. MS (ESI): mass calcd. For C₁₈H₁₅ClF₃N₅ 393.10, m/z found 394.2 [M+H]⁺.

### Intermediate-L22: 4, 7-difluoro-3-(7-(5-fluoropyrimidin-2-yl)-4, 7-diazaspiro [2.5]octan-4-yl)-1H-indazole

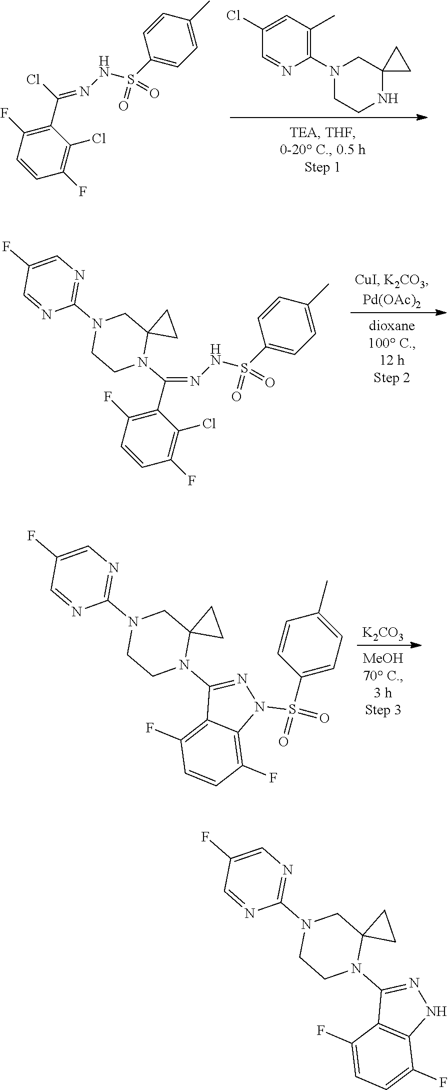

Step 1: To a solution of 7-(5-fluoropyrimidin-2-yl)-4, 7-diazaspiro [2.5]octane (1.36 g, 5.54 mmol, 0.7 eq, HCl) in THF (30 mL) was added dropwise TEA (2.40 g, 23.7 mmol, 3.30 mL, 3 eq) at 20° C. After addition, the mixture was stirred at this temperature for 5 minutes, and then (1Z)-2-chloro-3, 6-difluoro-N-(p-tolylsulfonyl) benzohydrazonoyl chloride (3.00 g, 7.91 mmol, 1 eq) in THF (6 mL) was added dropwise at 0° C. The resulting mixture was stirred at 20° C. for 25 minutes. LC-MS showed 7-(5-fluoropyrimidin-2-yl)-4, 7-diazaspiro [2.5]octane was consumed completely and desired mass was detected. The crude was added H₂O (20 mL), and extracted with EtOAc (10 mL*3). The combined organic layers were washed with brine (10 mL), dried over Na₂SO₄, filtered and concentrated under reduced pressure to give (Z)-2-chloro-3, 6-difluoro-N′-tosylbenzohydrazonoyl chloride (3.6 g, crude) was obtained as a yellow oil. MS (ESI): mass calcd. For C₂₄H₂₂ClF₃N₆O₂S 550.12, m/z found 551.3 [M+H]⁺.

Step 2: A mixture of N—[(Z)-[(2-chloro-3, 6-difluoro-phenyl)-[7-(5-fluoropyrimidin-2-yl)-4, 7-diazaspiro [2.5]octan-4-yl]methylene]amino]-4-methyl-benzenesulfonamide (3.6 g, 6.53 mmol, 1 eq), CuI (124 mg, 653 μmol, 0.1 eq), Pd(OAc)₂ (293 mg, 1.31 mmol, 0.2 eq) and K₂CO₃ (2.26 g, 16.3 mmol, 2.5 eq) in dioxane (40 mL) was degassed and purged with N₂ for 3 times, and then the mixture was stirred at 100° C. for 12 hours under the atmosphere of nitrogen. LC-MS showed N—[(Z)-[(2-chloro-3, 6-difluoro-phenyl)-[7-(5-fluoropyrimidin-2-yl)-4, 7-diazaspiro [2.5]octan-4-yl]methylene]amino]-4-methyl-benzenesulfonamide was consumed completely and desired mass was detected. The crude was added H₂O (100 mL) and extracted with EtOAc (150 mL*3). The combined organic layers were washed with brine (100 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜10% EtOAc/petroleum ether gradient @80 mL/min) to give desired 4, 7-difluoro-3-(7-(5-fluoropyrimidin-2-yl)-4, 7-diazaspiro [2.5]octan-4-yl)-1-tosyl-1H-indazole (550 mg, 1.07 mmol, 16.4% yield) as a yellow solid. MS (ESI): mass calcd. For C₂₄H₂₁F₃N₆O₂S 514.14, m/z found 515.2 [M+H]⁺.

Step 3: To a solution of 4, 7-difluoro-3-[7-(5-fluoropyrimidin-2-yl)-4, 7-diazaspiro [2.5]octan-4-yl]-1-(p-tolylsulfonyl) indazole (550 mg, 1.07 mmol, 1 eq) in MeOH (10 mL) was added K₂CO₃ (739 mg, 5.34 mmol, 5 eq). The mixture was stirred at 70° C. for 3 hours. LC-MS showed 4, 7-difluoro-3-[7-(5-fluoropyrimidin-2-yl)-4, 7-diazaspiro [2.5]octan-4-yl]-1-(p-tolylsulfonyl) indazole was consumed completely and desired mass was detected. The crude was added H₂O (10 mL) and extracted with EtOAc (15 mL*3). The combined organic layers were washed with brine (10 mL*2), dried over anhydrous Na₂SO₄, filtered and concentrated under reduced pressure to give a residue. The residue was purified by prep-TLC (SiO₂, petroleum ether/EtOAc=3/1) to give desired 4, 7-difluoro-3-(7-(5-fluoropyrimidin-2-yl)-4, 7-diazaspiro [2.5]octan-4-yl)-1H-indazole (180 mg, 496 μmol, 46.7% yield) as a yellow oil. MS (ESI): mass calcd. For C₁₇H₁₅F₃N₆ 360.13, m/z found 361.2 [M+H]⁺.

### Intermediate-L23: (Z)-4-chloro-2, 6-difluoro-N′-tosylbenzohydrazonoyl chloride

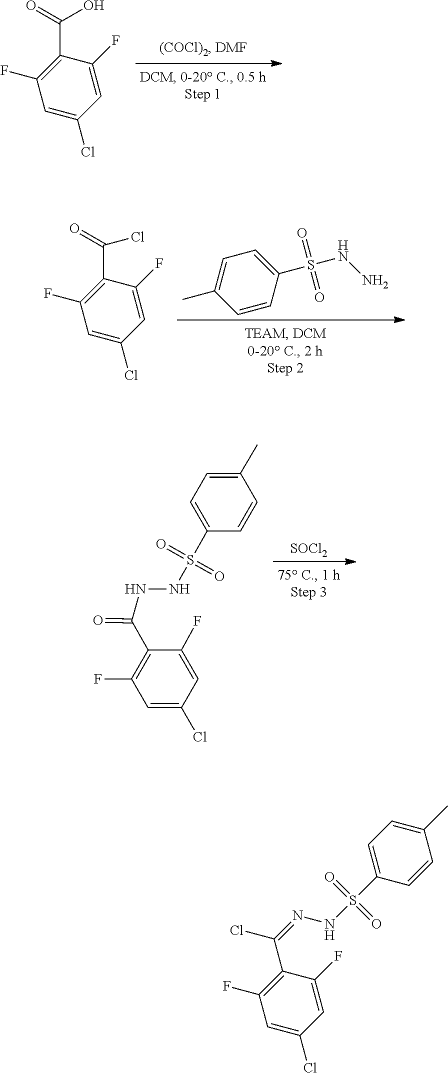

Step 1: To the mixture of 4-chloro-2,6-difluoro-benzoic acid (5 g, 26.0 mmol, 1 eq) in DCM (40 mL) and DMF (569 mg, 7.79 mmol, 0.3 eq) was added (COCl)₂ (9.89 g, 77.9 mmol, 6.82 mL, 3 eq) at 0° C. and the solution was stirred at 20° C. for 0.5 hour. TLC (SiO₂ petroleum ether/EtOAc=3/1) indicated 4-chloro-2, 6-difluoro-benzoic acid was consumed completely and one new spot formed. The reaction mixture was concentrated in vacuum to give 4-chloro-2, 6-difluoro-benzoyl chloride (5.5 g, crude) as a yellow solid.

Step 2: To the solution of 4-methylbenzenesulfonohydrazide (5.34 g, 28.7 mmol, 1.1 eq) and TEA (10.55 g, 104 mmol, 14.5 mL, 4 eq) in DCM (40 mL) was added the solution of 4-chloro-2, 6-difluoro-benzoyl chloride (5.5 g, 26.1 mmol, 1 eq) in DCM (10 mL) at 0° C. and the solution was stirred at 20° C. for 2 hours. LC-MS showed 4-methylbenzenesulfonohydrazide was consumed completely and desired mass was detected. The reaction was concentrated to get a residue. The residue was added water (50 mL) and extracted with EtOAc (3*20 mL). The combined organics were concentrated to get a residue. The residue was purified by flash silica gel chromatography (ISCO®; 40 g SepaFlash® Silica Flash Column, Eluent of 0˜50% ethyl acetate/petroleum ether gradient @80 mL/min) to give desired 4-chloro-2, 6-difluoro-N′-(p-tolylsulfonyl) benzohydrazide (1.54 g, 4.27 mmol, 16.4% yield) as a white solid. MS (ESI): mass calcd. For C₁₄H₁₁ClF₂N₂O₃S 360.04 m/z found 360.9 [M+H]⁺.

Step 3: The solution of 4-chloro-2, 6-difluoro-N′-(p-tolylsulfonyl) benzohydrazide (660 mg, 1.83 mmol, 1 eq) in SOCl₂ (6 mL) was stirred at 75° C. for 1 hour. TLC (SiO₂, petroleum ether/EtOAc=3/1) showed 4-chloro-2, 6-difluoro-N′-(p-tolylsulfonyl) benzohydrazide was consumed completely and a new spot with lower priority was detected. The reaction mixture was concentrated in vacuum to give desired (1Z)-4-chloro-2, 6-difluoro-N-(p-tolylsulfonyl) benzohydrazonoyl chloride (690 mg, crude) as an off-white solid.

### Intermediate-L24: (Z)-2, 6-dichloro-3-fluoro-N′-tosylbenzohydrazonoyl chloride

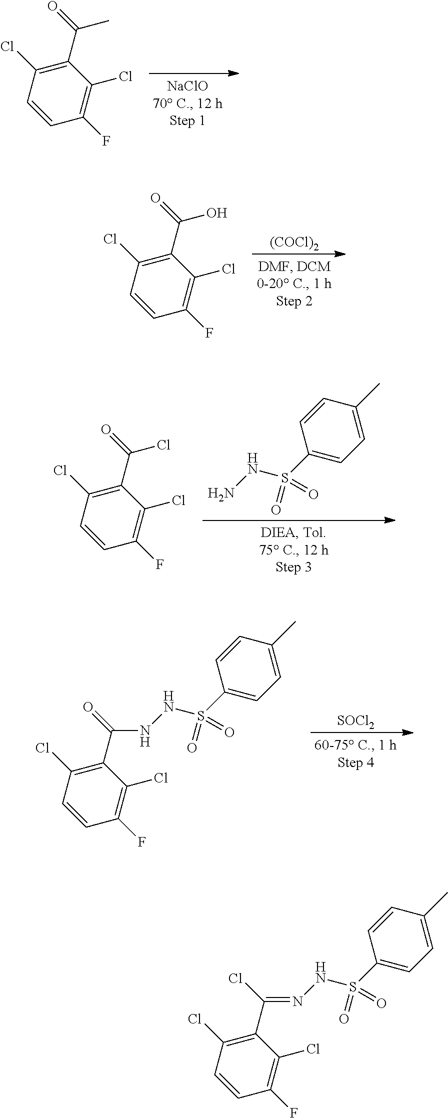

Step 1: A mixture of 1-(2, 6-dichloro-3-fluoro-phenyl) ethanone (5 g, 24.2 mmol, 3.57 mL, 1 eq) in NaClO (75 mL, 10% purity) was stirred at 70° C. for 12 hours. TLC (SiO₂, petroleum ether/EtOAc=4/1) showed a little of tert-butyl 1-(2, 6-dichloro-3-fluoro-phenyl)ethanone remained and a new spot formed. The temperature was cooled to room temperature, and 25 g of dichloromethane was added to extract unreacted materials. The layers were separated. The aqueous phase was neutralized with about 20 g of hydrochloric acid to pH value was 3, cooled, filtered, and dried at 80° C. give desired 2, 6-dichloro-3-fluorobenzoic acid (400 mg, crude) as a white solid.

Step 2: To a solution of 2, 6-dichloro-3-fluoro-benzoic acid (400 mg, 1.91 mmol, 1 eq) in DCM (5 mL) was added DMF (14.0 mg, 191 μmol, 14.7 μL, 0.1 eq) dropwise at 0° C. After addition, and then (COCl)₂ (486 mg, 3.83 mmol, 335 μL, 2 eq) was added dropwise at 0° C. The resulting mixture was stirred at 20° C. for 1 hour. TLC (SiO₂, petroleum ether/EtOAc=4/1) showed 2, 6-dichloro-3-fluoro-benzoic acid was consumed completely. The reaction mixture was concentrated to give desired 2, 6-dichloro-3-fluorobenzoyl chloride (435 mg, crude) as a yellow solid.

Step 3: To a solution of 4-methylbenzenesulfonohydrazide (356 mg, 1.91 mmol, 1 eq) in Tol. (5 mL) was added DIEA (297 mg, 2.30 mmol, 400 μL, 1.2 eq) and 2, 6-dichloro-3-fluoro-benzoyl chloride (435 mg, 1.91 mmol, 1 eq). The mixture was stirred at 75° C. for 12 hours. LC-MS showed 4-methylbenzenesulfonohydrazide was consumed completely and desired mass was detected. The crude was added H₂O (10 mL) and extracted with EtOAc (15 mL*3). The combined organic layers were washed with brine (10 mL*2), dried over anhydrous Na₂SO₄, filtered and concentrated under reduced pressure to give a residue. The residue was purified by prep-TLC (SiO₂, petroleum ether/EtOAc=3/1) to give desired N′-(2, 6-dichloro-3-fluorobenzoyl)-4-methylbenzenesulfonohydrazide (200 mg, 530 μmol, 27.7% yield) as a white solid. MS (ESI): mass calcd. For C₁₄H₁₁C₁₂FN₂O₃S 375.99, m/z found 376.9 [M+H]⁺.

Step 4: To a solution of 2, 6-dichloro-3-fluoro-N′-(p-tolylsulfonyl) benzohydrazide (150 mg, 398 μmol, 1 eq) and SOCl₂ (3 mL) was stirred at 75° C. for 0.5 hour. The reaction was cooled to 60° C. and an additional portion of 2, 6-dichloro-3-fluoro-N′-(p-tolylsulfonyl) benzohydrazide (150 mg, 398 μmol, 1 eq) was added and the reaction heated back to 75° C. for 0.5 hour. LC-MS (the simple was quenched with piperidine) showed 2, 6-dichloro-3-fluoro-N′-(p-tolylsulfonyl) benzohydrazide was consumed completely and desired mass was detected. The reaction mixture was concentrated to give desired (Z)-2, 6-dichloro-3-fluoro-N′-tosylbenzohydrazonoyl chloride (300 mg, crude) as a white solid. MS (ESI): mass calcd. For C₁₄H₁₀C₁₃FN₂O₂S 393.95, m/z found 444.0 [M+H+50]⁺.

### Intermediate-L25: 4-chloro-3-(7-(3, 5-difluoropyridin-2-yl)-4, 7-diazaspiro [2.5]octan-4-yl)-7-fluoro-1H-indazole

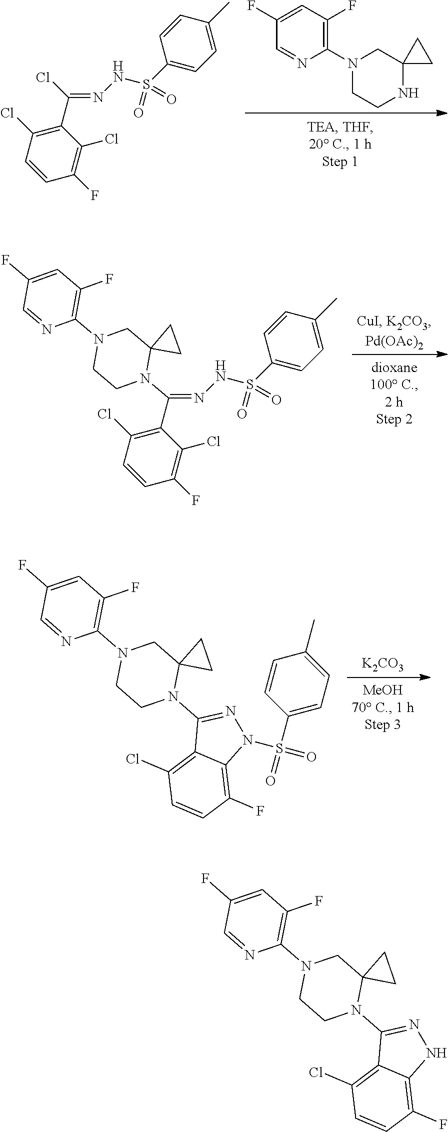

Step 1: To a solution of (1Z)-2, 6-dichloro-3-fluoro-N-(p-tolylsulfonyl)benzohydrazonoyl chloride (1.35 g, 3.41 mmol, 1.2 eq) in THF (5 mL) was added TEA (575 mg, 5.68 mmol, 791 μL, 2 eq) and 7-(3, 5-difluoro-2-pyridyl)-4, 7-diazaspiro [2.5]octane (640 mg, 2.84 mmol, 1 eq). The mixture was stirred at 20° C. for 1 hour. LC-MS showed (1Z)-2, 6-dichloro-3-fluoro-N-(p-tolylsulfonyl)benzohydrazonoyl chloride was consumed completely and desired mass was detected. The crude was added H₂O (40 mL), and extracted with EtOAc (30 mL*3). The combined organic layers were washed with brine (40 mL), dried over Na₂SO₄, filtered and concentrated under reduced pressure to give desired N—[(Z)-[(2, 6-dichloro-3-fluoro-phenyl)-[7-(3, 5-difluoro-2-pyridyl)-4, 7-diazaspiro [2.5]octan-4-yl]methylene]amino]-4-methyl-benzenesulfonamide (1.6 g, crude) as a yellow oil. MS (ESI): mass calcd. For C₂₅H₂₂C₁₂F₃N₅O₂S 583.08, m/z found 584.0 [M+H]⁺.

Step 2: A mixture of N—[(Z)-[(2, 6-dichloro-3-fluoro-phenyl)-[7-(3, 5-difluoro-2-pyridyl)-4, 7-diazaspiro [2.5]octan-4-yl]methylene]amino]-4-methyl-benzenesulfonamide (1.6 g, 2.74 mmol, 1 eq), CuI (52.1 mg, 274 μmol, 0.1 eq), Pd(OAc)₂ (123 mg, 548 μmol, 0.2 eq) and K₂CO₃ (946 mg, 6.84 mmol, 2.5 eq) in dioxane (20 mL) was degassed and purged with N₂ for 3 times, and then the mixture was stirred at 100° C. for 2 hours under the atmosphere of nitrogen. LC-MS showed N—[(Z)-[(2, 6-dichloro-3-fluoro-phenyl)-[7-(3, 5-difluoro-2-pyridyl)-4, 7-diazaspiro [2.5]octan-4-yl]methylene]amino]-4-methyl-benzenesulfonamide was consumed completely and desired mass was detected. The crude was added H₂O (10 mL) and extracted with EtOAc (15 mL*3). The combined organic layers were washed with brine (10 mL*2), dried over anhydrous Na₂SO₄, filtered and concentrated under reduced pressure to give desired 4-chloro-3-(7-(3, 5-difluoropyridin-2-yl)-4, 7-diazaspiro [2.5]octan-4-yl)-7-fluoro-1-tosyl-1H-indazole (1.5 g, crude) as a brown oil. MS (ESI): mass calcd. For C₂₅H₂₁ClF₃N₅O₂S 547.11, m/z found 548.1 [M+H]⁺.

Step 3: To a solution of 4-chloro-3-[7-(3, 5-difluoro-2-pyridyl)-4, 7-diazaspiro [2.5]octan-4-yl]-7-fluoro-1-(p-tolylsulfonyl) indazole (1.5 g, 2.74 mmol, 1 eq) in MeOH (15 mL) was added K₂CO₃ (1.89 g, 13.7 mmol, 5 eq). The mixture was stirred at 70° C. for 1 hour. LC-MS showed 4-chloro-3-[7-(3, 5-difluoro-2-pyridyl)-4, 7-diazaspiro [2.5]octan-4-yl]-7-fluoro-1-(p-tolylsulfonyl) indazole was consumed completely and desired mass was detected. The crude was added H₂O (10 mL) and extracted with EtOAc (15 mL*3). The combined organic layers were washed with brine (10 mL*2), dried over anhydrous Na₂SO₄, filtered and concentrated under reduced pressure to give a residue. The residue was purified by prep-TLC (SiO₂, petroleum ether/EtOAc=2/1) to give desired 4-chloro-3-(7-(3, 5-difluoropyridin-2-yl)-4, 7-diazaspiro [2.5]octan-4-yl)-7-fluoro-1H-indazole (390 mg, 990.37 μmol, 36.14% yield) as a brown oil. MS (ESI): mass calcd. For C₁₈H₁₅ClF₃N₅ 393.10, m/z found 394.0 [M+H]⁺.

### Intermediate-L26: 4-chloro-3-(7-(5-chloropyrimidin-2-yl)-4, 7-diazaspiro [2.5]octan-4-yl)-6-fluoro-1H-indazole

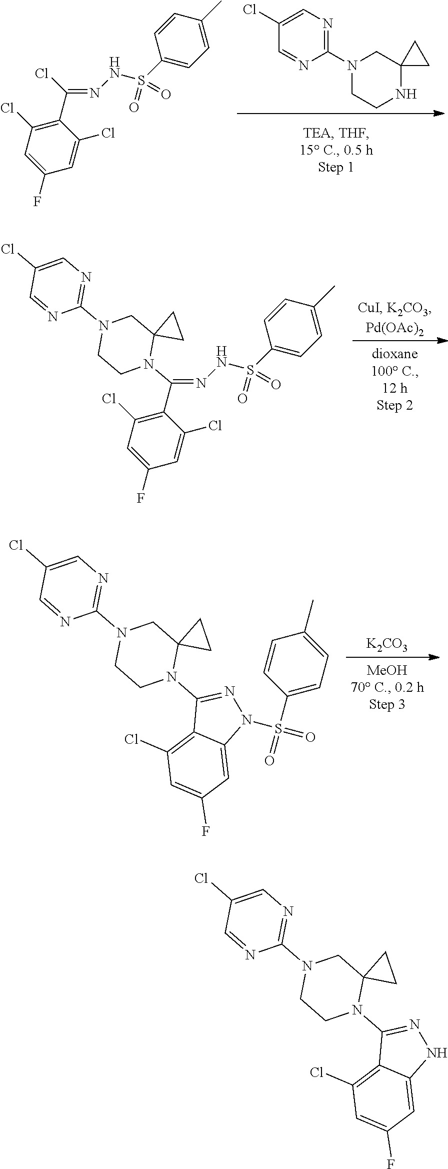

Step 1: To a solution of (1Z)-2, 6-dichloro-4-fluoro-N-(p-tolylsulfonyl)benzohydrazonoyl chloride (1.5 g, 3.79 mmol, 1 eq) in THF (20 mL) was added TEA (959 mg, 9.48 mmol, 1.32 mL, 2.5 eq) and 7-(5-chloropyrimidin-2-yl)-4,7-diazaspiro[2.5]octane (990 mg, 3.79 mmol, 1 eq, HCl). The mixture was stirred at 15° C. for 0.5 hour. LC-MS showed (1Z)-2, 6-dichloro-4-fluoro-N-(p-tolylsulfonyl) benzohydrazonoyl chloride was consumed completely and desired mass was detected. The crude was added H₂O (20 mL) and extracted with EtOAc (15 mL*3). The combined organic layers were washed with brine (20 mL), dried over anhydrous Na₂SO₄, filtered and concentrated under reduced pressure to give desired (Z)—N′-((7-(5-chloropyrimidin-2-yl)-4, 7-diazaspiro [2.5]octan-4-yl) (2, 6-dichloro-4-fluorophenyl) methylene)-4-methylbenzenesulfonohydrazide (2.2 g, crude) as a yellow solid. MS (ESI): mass calcd. For C₂₄H₂₂Cl₃FN₆O₂S 582.06, m/z found 583.0 [M+H]⁺.

Step 2: A mixture of N—[(Z)—[[7-(5-chloropyrimidin-2-yl)-4,7-diazaspiro[2.5]octan-4-yl]-(2,6-dichloro-4-fluoro-phenyl)methylene]amino]-4-methyl-benzenesulfonamide (2.2 g, 3.77 mmol, 1 eq), CuI (71.8 mg, 377 μmol, 0.1 eq), K₂CO₃ (1.30 g, 9.42 mmol, 2.5 eq) and Pd(OAc)₂ (170 mg, 754 μmol, 0.2 eq) in dioxane (20 mL) was degassed and purged with N₂ for 3 times, and then the mixture was stirred at 100° C. for 12 hours under the atmosphere of nitrogen. LC-MS showed N—[(Z)—[[7-(5-chloropyrimidin-2-yl)-4, 7-diazaspiro [2.5]octan-4-yl]-(2, 6-dichloro-4-fluoro-phenyl) methylene]amino]-4-methyl-benzenesulfonamide was consumed completely and desired mass was detected. The crude was added H₂O (40 mL) and extracted with EtOAc (30 mL*3). The combined organic layers were washed with brine (40 mL), dried over anhydrous Na₂SO₄, filtered and concentrated under reduced pressure to give desired 4-chloro-3-(7-(5-chloropyrimidin-2-yl)-4, 7-diazaspiro [2.5]octan-4-yl)-6-fluoro-1-tosyl-1H-indazole (2.0 g, crude) as a yellow solid. MS (ESI): mass calcd. For C₂₄H₂₁C₁₂FN₆O₂S 546.08, m/z found 547.1 [M+H]⁺.

Step 3: To a solution of 4-chloro-3-[7-(5-chloropyrimidin-2-yl)-4, 7-diazaspiro [2.5]octan-4-yl]-6-fluoro-1-(p-tolylsulfonyl) indazole (2 g, 3.65 mmol, 1 eq) in MeOH (20 mL) was added K₂CO₃ (2.52 g, 18.3 mmol, 5 eq). The mixture was stirred at 70° C. for 0.2 hour. LC-MS showed 4-chloro-3-[7-(5-chloropyrimidin-2-yl)-4, 7-diazaspiro [2.5]octan-4-yl]-6-fluoro-1-(p-tolylsulfonyl) indazole was consumed completely and desired mass was detected. The reaction mixture was concentrated under reduced pressure to give a residue. The crude was added H₂O (40 mL) and extracted with EtOAc (30 mL*3). The combined organic layers were washed with brine (40 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜35% EtOAc/petroleum ether gradient @100 mL/min) to give desired 4-chloro-3-(7-(5-chloropyrimidin-2-yl)-4, 7-diazaspiro [2.5]octan-4-yl)-6-fluoro-1H-indazole (60 mg, 153 μmol, 4.18% yield) as a yellow oil. MS (ESI): mass calcd. For C₁₇H₁₅C₁₂FN₆ 392.07, m/z found 393.0 [M+H]⁺.

### Intermediate-L27: 4-chloro-3-[7-(3, 5-difluoro-2-pyridyl)-4, 7-diazaspiro [2.5]octan-4-yl]-6-fluoro-1H-indazole

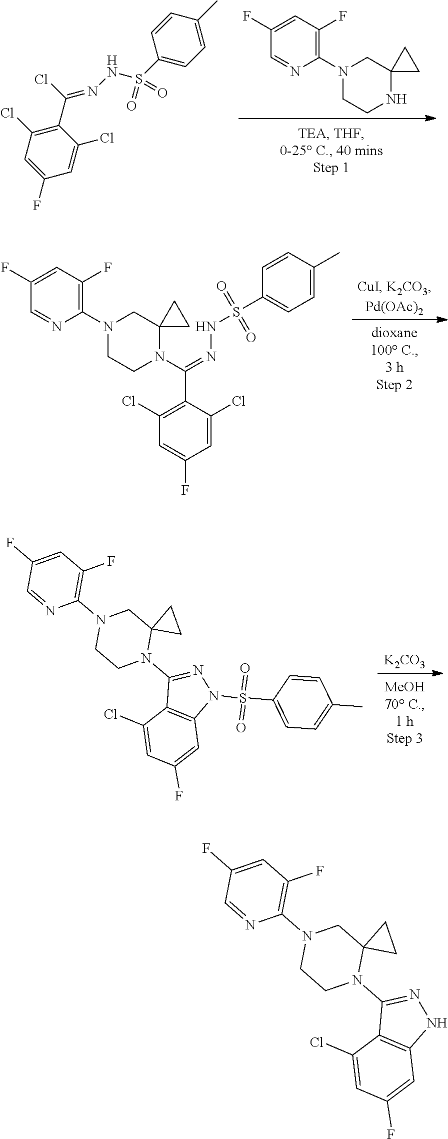

Step 1: To a solution of 7-(3, 5-difluoro-2-pyridyl)-4, 7-diazaspiro [2.5]octane (1.20 g, 4.60 mmol, 1 eq, HCl) in THF (10 mL) was added dropwise TEA (1.86 g, 18.4 mmol, 2.56 mL, 4 eq) at 25° C. After addition, the mixture was stirred at this temperature for 10 minutes, and then (1Z)-2, 6-dichloro-4-fluoro-N-(p-tolylsulfonyl) benzohydrazonoyl chloride (2.0 g, 5.05 mmol, 1.1 eq) was added dropwise at 0° C. The resulting mixture was stirred at 25° C. for 0.5 hour. LC-MS showed 7-(3, 5-difluoro-2-pyridyl)-4, 7-diazaspiro [2.5]octane was consumed completely and one main peak with desired mass was detected. Then it was separated between 20 mL of water and 40 mL of EtOAc. The organic phase was separated, washed with 30 mL of brine, dried over Na₂SO₄, filtered and concentrated under reduced pressure to give desired N—[(Z)-[(2, 6-dichloro-4-fluoro-phenyl)-[7-(3, 5-difluoro-2-pyridyl)-4, 7-diazaspiro [2.5]octan-4-yl]methylene]amino]-4-methyl-benzenesulfonamide (2.7 g, crude) as a yellow solid. MS (ESI): mass calcd. For C₂₅H₂₂C₁₂F₃N₅O₂S 583.08 m/z found 584.1 [M+H]⁺.

Step 2: A mixture of N—[(Z)-[(2, 6-dichloro-4-fluoro-phenyl)-[7-(3, 5-difluoro-2-pyridyl)-4, 7-diazaspiro [2.5]octan-4-yl]methylene]amino]-4-methyl-benzenesulfonamide (2.7 g, 4.62 mmol, 1 eq), CuI (88.0 mg, 462 μmol, 0.1 eq), Pd(OAc)₂ (207 mg, 924 μmol, 0.2 eq) and K₂CO₃ (1.60 g, 11.6 mmol, 2.5 eq) in dioxane (30 mL) was degassed and purged with N₂ for 3 times, and then the mixture was stirred at 100° C. for 3 hours under the atmosphere of nitrogen. LC-MS showed N—[(Z)-[(2, 6-dichloro-4-fluoro-phenyl)-[7-(3, 5-difluoro-2-pyridyl)-4, 7-diazaspiro [2.5]octan-4-yl]methylene]amino]-4-methyl-benzenesulfonamide was consumed completely and desired compound was detected. Then it was separated between 50 mL of water and 100 mL of EtOAc. The organic phase was separated, washed with 50 mL of brine, dried over Na₂SO₄, filtered and concentrated under reduced pressure to give desired 4-chloro-3-[7-(3, 5-difluoro-2-pyridyl)-4, 7-diazaspiro [2.5]octan-4-yl]-6-fluoro-1-(p-tolylsulfonyl) indazole (2.6 g, crude) as a black oil. MS (ESI): mass calcd. For C₂₅H₂₁ClF₃N₅O₂S 547.11 m/z found 548.1 [M+H]⁺.

Step 3: To a solution of 4-chloro-3-[7-(3, 5-difluoro-2-pyridyl)-4, 7-diazaspiro [2.5]octan-4-yl]-6-fluoro-1-(p-tolylsulfonyl) indazole (2.6 g, 4.74 mmol, 1 eq) in MeOH (20 mL) was added K₂CO₃ (3.28 g, 23.7 mmol, 5 eq). The mixture was stirred at 70° C. for 1 hour. LC-MS showed 4-chloro-3-[7-(3, 5-difluoro-2-pyridyl)-4, 7-diazaspiro [2.5]octan-4-yl]-6-fluoro-1-(p-tolylsulfonyl) indazole was consumed completely and desired compound was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by column chromatography (SiO₂, petroleum ether/EtOAc=1/0 to 1/1) to give desired 4-chloro-3-[7-(3,5-difluoro-2-pyridyl)-4, 7-diazaspiro [2.5]octan-4-yl]-6-fluoro-1H-indazole (800 mg, 2.03 mmol, 42.8% yield) as a yellow oil. MS (ESI): mass calcd. For C₁₈H₁₅ClF₃N₅ 393.10 m/z found 394.2 [M+H]⁺.

### Intermediate-L28: 2-(3, 8-diazabicyclo [3.2.1]octan-3-yl)-5-chlorothiazole

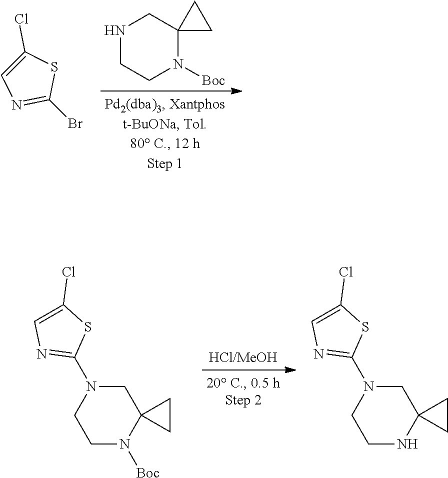 [SMILES: Clc1cnc(Br)s1 ; CC(C)(C)OC(=O)N1CCNCC12CC2 ; CC(C)(C)OC(=O)N1CCN(c2ncc(Cl)s2)CC12CC2 ; Clc1cnc(N2CCNC3(CC3)C2)s1]

Step 1: A mixture of 2-bromo-5-chloro-thiazole (3 g, 15.1 mmol, 1 eq), tert-butyl 4, 7-diazaspiro [2.5]octane-4-carboxylate (3.21 g, 15.1 mmol, 1 eq), Pd₂(dba)₃ (692 mg, 756 μmol, 0.05 eq), Xantphos (875 mg, 1.51 mmol, 0.1 eq) and t-BuONa (3.63 g, 37.8 mmol, 2.5 eq) in Tol. (30 mL) was degassed and purged with N₂ for 3 times, and then the mixture was stirred at 80° C. for 12 hours under the atmosphere of nitrogen. LCMS showed 2-bromo-5-chloro-thiazole was consumed completely and one main peak with desired mass. The residue was diluted with H₂O (100 mL) and extracted with EtOAc (50 mL*3). The combined organic layers were washed with brine (100 mL), dried over Na₂SO₄, filtered and concentrated under reduced pressure to give a residue. The residue was purified by flash silica gel chromatography (ISCO®; 80 g SepaFlash® Silica Flash Column, Eluent of 0˜16% EtOAc/petroleum ether gradient @100 mL/min) to give desired tert-butyl 7-(5-chlorothiazol-2-yl)-4, 7-diazaspiro [2.5]octane-4-carboxylate (4.7 g, 14.3 mmol, 94.3% yield) as a yellow oil. MS (ESI): mass calcd. For C₁₄H₂₀ClN₃O₂S 329.10, m/z found 330.0 [M+H]⁺.

Step 2: A mixture of tert-butyl 7-(5-chlorothiazol-2-yl)-4, 7-diazaspiro [2.5]octane-4-carboxylate (0.5 g, 1.52 mmol, 1 eq) in HCl/EtOAc (4 M, 5 mL, 13.2 eq) was degassed and purged with N₂ for 3 times, and then the mixture was stirred at 20° C. for 0.5 hour under the atmosphere of nitrogen. LC-MS showed tert-butyl 7-(5-chlorothiazol-2-yl)-4, 7-diazaspiro [2.5]octane-4-carboxylate was consumed completely and one main peak with desired mass was detected. The reaction mixture was concentrated in vacuum to give desired 2-(3, 8-diazabicyclo [3.2.1]octan-3-yl)-5-chlorothiazole (0.4 g, crude, HCl) as a yellow solid. MS (ESI): mass calcd. For C₉H₁₂ClN₃S 229.04 m/z found 230.0 [M+H]⁺.

### Intermediate-L29: 5-chloro-2-(4-(4-chloro-6-fluoro-1-tosyl-1H-indazol-3-yl)-4, 7-diazaspiro [2.5]octan-7-yl) thiazole

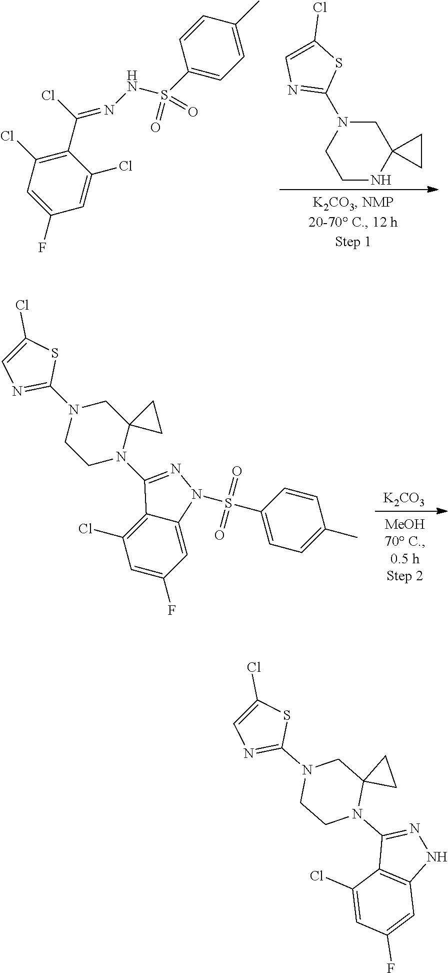

Step 1: To the solution of 5-chloro-2-(4, 7-diazaspiro [2.5]octan-7-yl)thiazole (808 mg, 3.04 mmol, 1.2 eq, HCl) and K₂CO₃ (1.40 g, 10.1 mmol, 1.41 mL, 4 eq) in NMP (3 mL) was added the solution of (1Z)-2, 6-dichloro-4-fluoro-N-(p-tolylsulfonyl) benzohydrazonoyl chloride (1 g, 2.53 mmol, 1 eq) in NMP (3 mL) at 20° C. and the solution was stirred at 20° C. for 1 hour. LCMS showed 5-chloro-2-(4, 7-diazaspiro [2.5]octan-7-yl) thiazole remained and one main peak with desired mass was detected. The reaction mixture was stirred at 70° C. for 11 hours. LCMS showed tert-butyl 7-(5-chlorothiazol-2-yl)-4, 7-diazaspiro [2.5]octane-4-carboxylate was consumed completely and one main peak with desired mass was detected. The reaction was added water (20 mL) and extracted with MTBE (2*20 mL). The combined organics were washed with brine (10 mL), dried over anhydrous sodium sulfate and filtered. The filtrate was concentrated to get a residue. The residue was purified by column chromatography (ISCO®; 20 g SepaFlash® Silica Flash Column, Eluent of 0˜10% EtOAc/petroleum ether gradient @60 mL/min) to give desired 5-chloro-2-(4-(4-chloro-6-fluoro-1-tosyl-1H-indazol-3-yl)-4, 7-diazaspiro [2.5]octan-7-yl) thiazole (200 mg, 362 μmol, 14.3% yield) as a yellow oil. MS (ESI): mass calcd. For C₂₃H₂₀C₁₂FN₅O₂S₂ 551.04, m/z found 552.1 [M+H]⁺.

Step 2: The solution of 5-chloro-2-[4-[4-chloro-6-fluoro-1-(p-tolylsulfonyl) indazol-3-yl]-4, 7-diazaspiro [2.5]octan-7-yl]thiazole (200 mg, 362 μmol, 1 eq) and K₂CO₃ (250 mg, 1.81 mmol, 5 eq) in MeOH (3 mL) was stirred at 70° C. for 0.5 hour. LCMS showed 5-chloro-2-[4-[4-chloro-6-fluoro-1-(p-tolylsulfonyl) indazol-3-yl]-4, 7-diazaspiro [2.5]octan-7-yl]thiazole was consumed completely and one main peak with desired mass was detected. The reaction was concentrated to get a residue. The residue was purified by prep-TLC (SiO₂, petroleum ether/ethyl acetate=1/1) to give desired 5-chloro-2-(4-(4-chloro-6-fluoro-1H-indazol-3-yl)-4, 7-diazaspiro [2.5]octan-7-yl) thiazole (130 mg, 326 μmol, 90.2% yield) as alight yellow oil. MS (ESI): mass calcd. For C₁₆H₁₄C₁₂FN₅S 397.03, m/z found 398.0 [M+H]⁺.

### Intermediate-L30: 4-chloro-6-fluoro-3-[7-(5-fluoro-2-pyridyl)-4, 7-diazaspiro [2.5]octan-4-yl]-1H-indazole

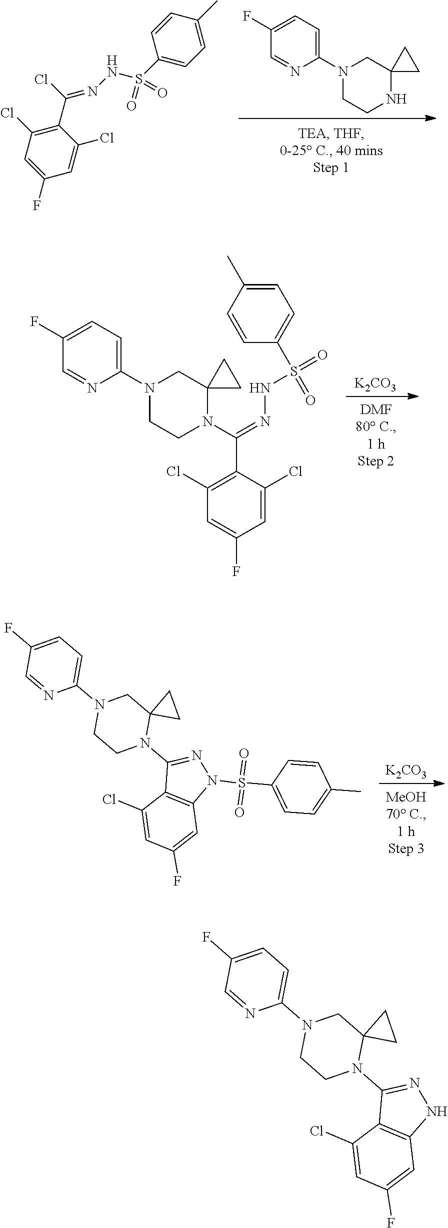

Step 1: To a solution of 7-(5-fluoro-2-pyridyl)-4, 7-diazaspiro [2.5]octane (400 mg, 1.64 mmol, 1 eq, HCl) in THF (10 mL) was added dropwise TEA (1.66 g, 16.4 mmol, 2.28 mL, 10 eq) at 25° C. After addition, the mixture was stirred at this temperature for 10 minutes, and then (1Z)-2, 6-dichloro-4-fluoro-N-(p-tolylsulfonyl)benzohydrazonoyl chloride (714 mg, 1.81 mmol, 1.1 eq) in THF (5 mL) was added dropwise at 0° C. The resulting mixture was stirred at 25° C. for 0.5 hour. LC-MS showed 7-(5-fluoro-2-pyridyl)-4, 7-diazaspiro [2.5]octane was consumed completely and one main peak with desired mass was detected. Then it was separated between 20 mL of water and 40 mL of EtOAc. The organic phase was separated, washed with 30 mL of brine, dried over Na₂SO₄, filtered and concentrated under reduced pressure to give desired N—[(Z)-[(2, 6-dichloro-4-fluoro-phenyl)-[7-(5-fluoro-2-pyridyl)-4, 7-diazaspiro [2.5]octan-4-yl]methylene]amino]-4-methyl-benzenesulfonamide (930 mg, crude) as a yellow solid. MS (ESI): mass calcd. For C₂₅H₂₃C₁₂F₂N₅O₂S 565.09, m/z found 566.2 [M+H]⁺.

Step 2: To a solution of N—[(Z)-[(2, 6-dichloro-4-fluoro-phenyl)-[7-(5-fluoro-2-pyridyl)-4, 7-diazaspiro [2.5]octan-4-yl]methylene]amino]-4-methyl-benzenesulfonamide (930 mg, 1.64 mmol, 1 eq) in DMF (10 mL) was added K₂CO₃ (908 mg, 6.57 mmol, 4 eq). The mixture was stirred at 80° C. for 1 hour. LC-MS showed N—[(Z)-[(2, 6-dichloro-4-fluoro-phenyl)-[7-(5-fluoro-2-pyridyl)-4, 7-diazaspiro [2.5]octan-4-yl]methylene]amino]-4-methyl-benzenesulfonamide was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give desired 4-chloro-6-fluoro-3-[7-(5-fluoro-2-pyridyl)-4, 7-diazaspiro [2.5]octan-4-yl]-1-(p-tolylsulfonyl) indazole (870 mg, crude) as a black oil. MS (ESI): mass calcd. For C₂₅H₂₂ClF₂N₅O₂S 529.12, m/z found 530.1 [M+H]⁺.

Step 3: To a solution of 4-chloro-6-fluoro-3-[7-(5-fluoro-2-pyridyl)-4, 7-diazaspiro [2.5]octan-4-yl]-1-(p-tolylsulfonyl) indazole (870 mg, 1.64 mmol, 1 eq) in MeOH (20 mL) was added K₂CO₃ (1.13 g, 8.21 mmol, 5 eq). The mixture was stirred at 70° C. for 1 hour. LC-MS showed 4-chloro-6-fluoro-3-[7-(5-fluoro-2-pyridyl)-4, 7-diazaspiro [2.5]octan-4-yl]-1-(p-tolylsulfonyl) indazole was consumed completely and desired compound was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by column chromatography (SiO₂, petroleum ether/EtOAc=1/0 to 1/1) to give desired 4-chloro-6-fluoro-3-[7-(5-fluoro-2-pyridyl)-4, 7-diazaspiro [2.5]octan-4-yl]-1H-indazole (490 mg, 1.30 mmol, 79.4% yield) as a yellow oil. MS (ESI): mass calcd. For C₁₈H₁₆ClF₂N₅ 375.11, m/z found 376.1 [M+H]⁺.

### Intermediate-L31: 5-chloro-2-(3-cyclopropylpiperazin-1-yl) pyrimidine

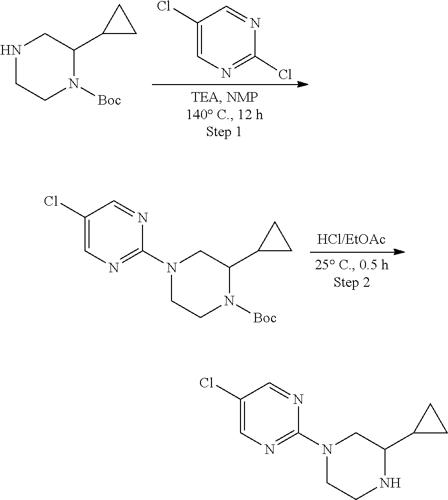

Step 1: To a solution of tert-butyl 2-cyclopropylpiperazine-1-carboxylate (501 mg, 2.22 mmol, 1.1 eq) and 2, 5-dichloropyrimidine (300 mg, 2.01 mmol, 1.0 eq) in NMP (6 mL) was added TEA (611 mg, 6.04 mmol, 841 μL, 3 eq). The mixture was stirred at 140° C. for 12 hours. LC-MS showed tert-butyl 2-cyclopropylpiperazine-1-carboxylate was consumed completely and desired mass was detected. The reaction mixture was partitioned between 20 mL of H₂O and 30 mL of EtOAc. The organic phase was separated, washed with 50 mL of brine, dried over Na₂SO₄, filtered and concentrated under reduced pressure to give the crude product. The residue was purified by flash silica gel chromatography (ISCO®; 12 g, SepaFlash® Silica Flash Column, Eluent of 0˜15% Ethyl acetate/petroleum ethergradient @60 mL/min) to give desired tert-butyl 4-(5-chloropyrimidin-2-yl)-2-cyclopropyl-piperazine-1-carboxylate (670 mg, 1.98 mmol, 98.20% yield) as a white solid. MS (ESI): mass calcd. For C₁₆H₂₃ClN₄O₂ 338.10, m/z found 339.1 [M+H]⁺.

Step 2: To a solution of tert-butyl 4-(5-chloropyrimidin-2-yl)-2-cyclopropyl-piperazine-1-carboxylate (670 mg, 1.98 mmol, 1 eq) in HCl/EtOAc (4M, 7 mL). The mixture was stirred at 25° C. for 0.5 hour. LC-MS showed tert-butyl 4-(5-chloropyrimidin-2-yl)-2-cyclopropyl-piperazine-1-carboxylate was consumed completely and desired mass was detected. The reaction mixture was filtered and the filter liquor was concentrated under reduced pressure to give desired 5-chloro-2-(3-cyclopropylpiperazin-1-yl) pyrimidine (540 mg, crude) as a white solid. MS (ESI): mass calcd. For C₁₁H₁₅ClN₄ 238.10, m/z found 239.1 [M+H]⁺.

### Intermediate-L32: 4-chloro-3-(7-(5-fluoropyridin-2-yl)-4, 7-diazaspiro [2.5]octan-4-yl)-1H-indazole

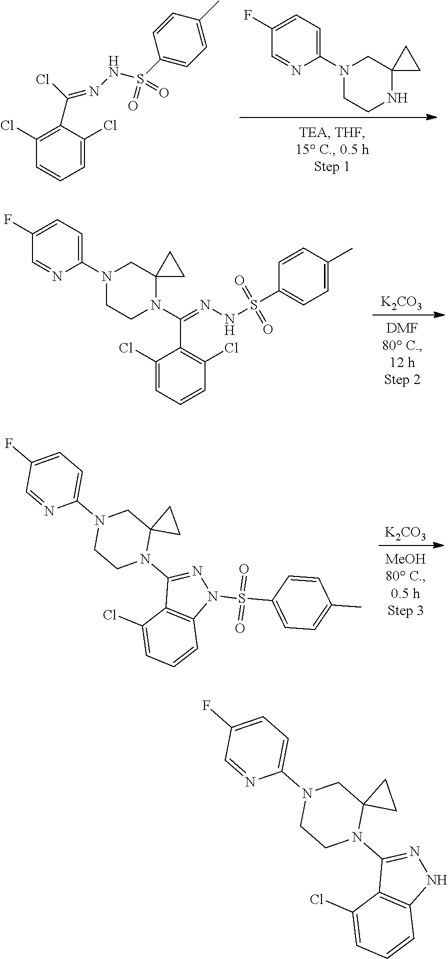

Step 1: To a solution of 7-(5-fluoro-2-pyridyl)-4,7-diazaspiro [2.5]octane (400 mg, 1.64 mmol, 1 eq, HCl) in THF (10 mL) was added TEA (415 mg, 4.10 mmol, 571 μL, 2.5 eq and (1Z)-2,6-dichloro-N-(p-tolylsulfonyl)benzohydrazonoyl chloride (744 mg, 1.97 mmol, 1.2 eq. The mixture was stirred at 15° C. for 0.5 hour. LC-MS showed 7-(5-fluoro-2-pyridyl)-4, 7-diazaspiro [2.5]octane was consumed completely and desired mass was detected. The crude was added H₂O (10 mL), and extracted with EtOAc (45 mL). The combined organic layers were washed with brine (10 mL), dried over Na₂SO₄, filtered and concentrated under reduced pressure to give a residue (E)-N′-((2, 6-dichlorophenyl) (7-(5-fluoropyridin-2-yl)-4, 7-diazaspiro [2.5]octan-4-yl) methylene)-4-methylbenzenesulfonohydrazide (770 mg, crude) as a brown oil. MS (ESI): mass calcd. For C₂₅H₂₄C₁₂FN₅O₂S 547.10, m/z found 548.1 [M+H]⁺.

Step 2: To a solution of N—[(Z)-[(2, 6-dichlorophenyl)-[7-(5-fluoro-2-pyridyl)-4, 7-diazaspiro [2.5]octan-4-yl]methylene]amino]-4-methyl-benzenesulfonamide (770 mg, 1.40 mmol, 1 eq) in DMF (10 mL) was added K₂CO₃ (1.94 g, 14.0 mmol, 10 eq). The mixture was stirred at 80° C. for 12 hours. LC-MS showed N—[(Z)-[(2, 6-dichloro-4-fluoro-phenyl)-[8-(5-fluoropyrimidin-2-yl)-5, 8-diazaspiro [3.5]nonan-5-yl]methylene]amino]-4-methyl-benzenesulfonamide was consumed completely and desired mass was detected. The crude was added H₂O (20 mL), and extracted with EtOAc (45 mL). The combined organic layers were washed with brine (10 mL), dried over Na₂SO₄, filtered and concentrated under reduced pressure to give a residue 4-chloro-3-(7-(5-fluoropyridin-2-yl)-4, 7-diazaspiro [2.5]octan-4-yl)-1-tosyl-1H-indazole (750 mg, crude) as a brown oil. MS (ESI): mass calcd. For C₂₅H₂₃ClFN₅O₂S 511.12, m/z found 512.1 [M+H]⁺.

Step 3: To a solution of 4-chloro-3-[7-(5-fluoro-2-pyridyl)-4, 7-diazaspiro [2.5]octan-4-yl]-1-(p-tolylsulfonyl) indazole (750 mg, 1.46 mmol, 1 eq) in MeOH (10 mL) was added K₂CO₃ (1.01 g, 7.32 mmol, 5 eq). The mixture was stirred at 80° C. for 0.5 hour. LC-MS showed 4-chloro-3-[7-(5-fluoro-2-pyridyl)-4, 7-diazaspiro [2.5]octan-4-yl]-1-(p-tolylsulfonyl) indazole was consumed completely and desired mass was detected. The reaction mixture was concentrated under reduced pressure to give a residue. The crude was added H₂O (20 mL), and extracted with EtOAc (45 mL). The combined organic layers were washed with brine (10 mL), dried over Na₂SO₄, filtered and concentrated under reduced pressure to give a residue. The residue was purified by column prep-TLC (SiO₂, petroleum ether/ethyl acetate=2/1) to give desired 4-chloro-3-(7-(5-fluoropyridin-2-yl)-4, 7-diazaspiro [2.5]octan-4-yl)-1H-indazole (110 mg, 307 μmol, 21.0% yield) as a yellow oil. MS (ESI): mass calcd. For C₁₈H₁₇ClFN₅ 357.12, m/z found 358.1 [M+H]⁺.

### Intermediate-L33: 7-(5-chloropyridin-2-yl)-4, 7-diazaspiro [2.5]octane

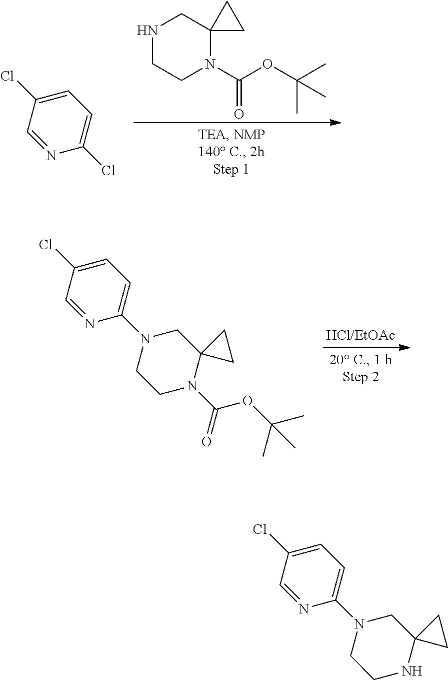 [SMILES: Clc1ccc(Cl)nc1 ; CC(C)(C)OC(=O)N1CCNCC12CC2 ; CC(C)(C)OC(=O)N1CCN(c2ccc(Cl)cn2)CC12CC2 ; Clc1ccc(N2CCNC3(CC3)C2)nc1]

Step 1: To a solution of 2, 5-dichloropyridine (700 mg, 4.73 mmol, 1 eq) and tert-butyl 4, 7-diazaspiro [2.5]octane-4-carboxylate (1.10 g, 5.20 mmol, 1.1 eq) in NMP (10 mL) was added TEA (1.44 g, 14.2 mmol, 1.98 mL, 3 eq). The mixture was stirred at 140° C. for 2 hours. LC-MS showed 2, 5-dichloropyridine was consumed completely and one main peak with desired mass was detected. The reaction mixture was diluted with H₂O (10 mL) and extracted with EtOAc (15 mL*2). The combined organic layers were washed with brine (5 mL*2), dried over anhydrous Na₂SO₄, filtered and concentrated under reduced pressure to give a residue. The residue was purified by flash silica gel chromatography (ISCO®; 40 g, SepaFlash® Silica Flash Column, Eluent of 0˜30% ethyl acetate/petroleum ethergradient @40 mL/min) to give desired tert-butyl 7-(5-chloropyridin-2-yl)-4, 7-diazaspiro [2.5]octane-4-carboxylate (400 mg, 1.24 mmol, 26.12% yield) as a colorless oil. MS (ESI): mass calcd. For C₁₆H₂₂N₃ClO₂ 323.14, m/z found 324.1 [M+H]⁺.

Step 2: To a solution of tert-butyl 7-(5-chloro-2-pyridyl)-4, 7-diazaspiro [2.5]octane-4-carboxylate (400 mg, 1.24 mmol, 1 eq) in HCl/EtOAc (4 M, 10 mL). The mixture was stirred at 20° C. for 1 hour. TLC showed tert-butyl 7-(5-chloro-2-pyridyl)-4, 7-diazaspiro [2.5]octane-4-carboxylate was consumed completely and one new spot formed. The reaction mixture was concentrated to give desired 7-(5-chloro-2-pyridyl)-4, 7-diazaspiro [2.5]octane (320 mg, crude, HCl salt) as a white solid.

### Intermediate-L34: 4-chloro-3-[7-(3, 5-difluoro-2-pyridyl)-4, 7-diazaspiro [2.5]octan-4-yl]-1H-indazole

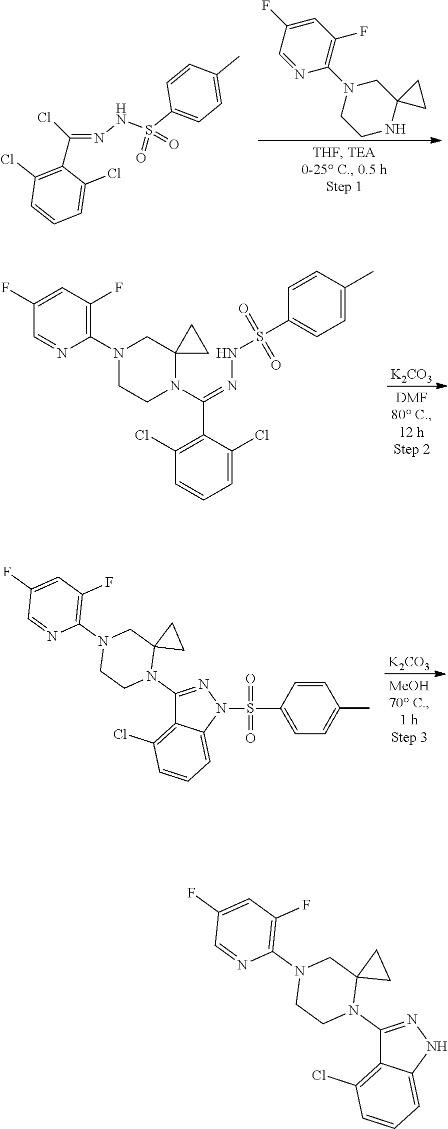

Step 1: To a solution of 7-(3,5-difluoro-2-pyridyl)-4,7-diazaspiro[2.5]octane (252 mg, 964 μmol, 1 eq, HCl) in THF (5 mL) was added dropwise TEA (975 mg, 9.64 mmol, 1.34 mL, 10 eq) at 25° C. After addition, the mixture was stirred at this temperature for 10 minutes, and then (1Z)-2,6-dichloro-N-(p-tolylsulfonyl) benzohydrazonoyl chloride (400 mg, 1.06 mmol, 1.1 eq) in THF (3 mL) was added dropwise at 0° C. The resulting mixture was stirred at 25° C. for 20 minutes. LC-MS showed 7-(3, 5-difluoro-2-pyridyl)-4, 7-diazaspiro [2.5]octane was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N—[(Z)-[(2,6-dichlorophenyl)-[7-(3,5-difluoro-2-pyridyl)-4,7-diazaspiro[2.5]octan-4-yl]methylene]amino]-4-methyl-benzenesulfonamide (700 mg, crude) as a yellow solid. MS (ESI): mass calcd. For C₂₅H₂₃C₁₂F₂N₅O₂S 565.09, m/z found 566.1 [M+H]⁺.

Step 2: To a solution of N—[(Z)-[(2, 6-dichlorophenyl)-[7-(3, 5-difluoro-2-pyridyl)-4, 7-diazaspiro [2.5]octan-4-yl]methylene]amino]-4-methyl-benzenesulfonamide (700 mg, 1.24 mmol, 1 eq) in DMF (8 mL) was added K₂CO₃ (854 mg, 6.18 mmol, 5 eq). The mixture was stirred at 80° C. for 12 hours. LC-MS showed N—[(Z)-[(2,6-dichlorophenyl)-[7-(3,5-difluoro-2-pyridyl)-4,7-diazaspiro[2.5]octan-4-yl]methylene]amino]-4-methyl-benzenesulfonamide was consumed completely and desired mass was detected. The reaction mixture was partitioned between 20 mL of H₂O and 20 mL of EtOAc. The organic phase was separated, washed with 50 mL of brine, dried over Na₂SO₄, filtered and concentrated under reduced pressure to give desired 4-chloro-3-[7-(3, 5-difluoro-2-pyridyl)-4, 7-diazaspiro [2.5]octan-4-yl]-1-(p-tolylsulfonyl)indazole (700 mg, crude) as a yellow oil. MS (ESI): mass calcd. For C₂₅H₂₂ClF₂N₅O₂S 529.12, m/z found 530.1 [M+H]⁺.

Step 3: To a solution of 4-chloro-3-[7-(3, 5-difluoro-2-pyridyl)-4, 7-diazaspiro [2.5]octan-4-yl]-1-(p-tolylsulfonyl) indazole (700 mg, 1.32 mmol, 1 eq) in MeOH (6 mL) was added K₂CO₃ (913 mg, 6.60 mmol, 5 eq). The mixture was stirred at 70° C. for 1 hour. LC-MS showed 4-chloro-3-[7-(3, 5-difluoro-2-pyridyl)-4, 7-diazaspiro [2.5]octan-4-yl]-1-(p-tolylsulfonyl) indazole was consumed completely and desired mass was detected. The reaction mixture was filtered and the filter liquor was concentrated under reduced pressure to give a residue. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether/ethyl acetate=2/1) to give desired 4-chloro-3-[7-(3, 5-difluoro-2-pyridyl)-4, 7-diazaspiro [2.5]octan-4-yl]-1H-indazole (150 mg, 399 μmol, 30.2% yield) as a yellow oil. MS (ESI): mass calcd. For C₁₈H₁₆ClF₂N₅ 375.11, m/z found 376.1 [M+H]⁺.

### Intermediate-L35: 3-(7-(3, 5-difluoropyridin-2-yl)-4, 7-diazaspiro [2.5]octan-4-yl)-4, 6-difluoro-1H-indazole

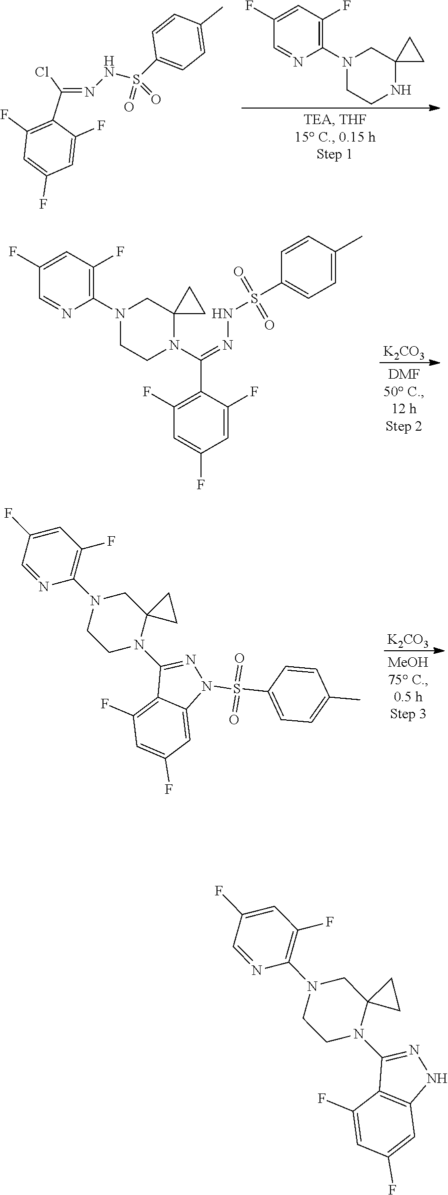

Step 1: To a solution of (1Z)-2, 4, 6-trifluoro-N-(p-tolylsulfonyl)benzohydrazonoyl chloride (1 g, 2.76 mmol, 1.2 eq) in THF (10 mL) was added TEA (581 mg, 5.74 mmol, 799 μL, 2.5 eq) and 7-(3, 5-difluoro-2-pyridyl)-4, 7-diazaspiro [2.5]octane (601 mg, 2.30 mmol, 1 eq, HCl). The mixture was stirred at 15° C. for 0.15 hour. LC-MS showed (1Z)-2, 4, 6-trifluoro-N-(p-tolylsulfonyl) benzohydrazonoyl chloride was consumed completely and desired mass was detected. The crude was added H₂O (10 mL), and extracted with EtOAc (45 mL). The combined organic layers were washed with brine (10 mL), dried over Na₂SO₄, filtered and concentrated under reduced pressure to give desired (Z)—N′-((7-(3, 5-difluoropyridin-2-yl)-4, 7-diazaspiro [2.5]octan-4-yl) (2, 4, 6-trifluorophenyl) methylene)-4-methylbenzenesulfonohydrazide (1.27 g, crude) as a yellow solid. MS (ESI): mass calcd. For C₂₅H₂₂F₅N₅O₂S 551.14, m/z found 552.2 [M+H]⁺.

Step 2: A mixture of N—[(Z)—[[7-(3, 5-difluoro-2-pyridyl)-4, 7-diazaspiro [2.5]octan-4-yl]-(2, 4, 6-trifluorophenyl) methylene]amino]-4-methyl-benzenesulfonamide (1.27 g, 2.30 mmol, 1 eq), K₂CO₃ (3.18 g, 23.0 mmol, 10 eq) in DMF (15 mL) was degassed and purged with N₂ for 3 times, and then the mixture was stirred at 50° C. for 12 hours under the atmosphere of nitrogen. LC-MS showed N—[(Z)—[[7-(3, 5-difluoro-2-pyridyl)-4, 7-diazaspiro [2.5]octan-4-yl]-(2, 4, 6-trifluorophenyl) methylene]amino]-4-methyl-benzenesulfonamide was consumed completely and desired mass was detected. The crude was added H₂O (10 mL), and extracted with EtOAc (45 mL). The combined organic layers were washed with brine (10 mL), dried over Na₂SO₄, filtered and concentrated under reduced pressure to give desired 3-(7-(3, 5-difluoropyridin-2-yl)-4, 7-diazaspiro [2.5]octan-4-yl)-4, 6-difluoro-1-tosyl-1H-indazole (1.2 g, crude) as a brown solid. MS (ESI): mass calcd. For C₂₅H₂₁F₄N₅O₂S 531.14, m/z found 532.2 [M+H]⁺.

Step 3: To a solution of 3-[7-(3, 5-difluoro-2-pyridyl)-4, 7-diazaspiro [2.5]octan-4-yl]-4, 6-difluoro-1-(p-tolylsulfonyl) indazole (1.2 g, 2.26 mmol, 1 eq) in MeOH (10 mL) was added K₂CO₃ (1.56 g, 11.3 mmol, 5 eq). The mixture was stirred at 75° C. for 0.5 hour. LC-MS showed 3-[7-(3, 5-difluoro-2-pyridyl)-4, 7-diazaspiro [2.5]octan-4-yl]-4, 6-difluoro-1-(p-tolylsulfonyl) indazole was consumed completely and desired mass was detected. The reaction mixture was concentrated under reduced pressure to give a residue. The crude was added H₂O (10 mL), and extracted with EtOAc (45 mL). The combined organic layers were washed with brine (10 mL), dried over Na₂SO₄, filtered and concentrated under reduced pressure to give a residue. The residue was purified by column chromatography (SiO₂, petroleum ether/ethyl acetate=5/1 to 4/1) to give desired 3-(7-(3, 5-difluoropyridin-2-yl)-4, 7-diazaspiro [2.5]octan-4-yl)-4, 6-difluoro-1H-indazole (450 mg, 1.19 mmol, 52.8% yield) as a brown solid. MS (ESI): mass calcd. For C₁₈H₁₅F₄N₅ 377.13, m/z found 378.1 [M+H]⁺.

### Intermediate-L36: 2-(3-cyclopropylpiperazin-1-yl)-5-fluoropyrimidine

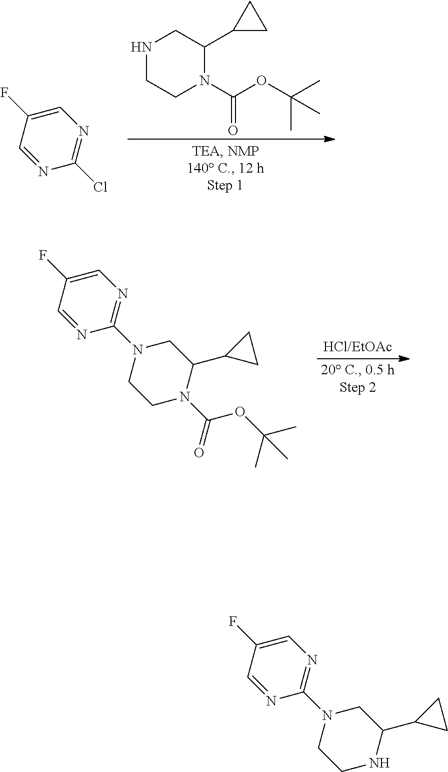 [SMILES: Fc1cnc(Cl)nc1 ; CC(C)(C)OC(=O)N1CCNCC1C1CC1 ; CC(C)(C)OC(=O)N1CCN(c2ncc(F)cn2)CC1C1CC1 ; Fc1cnc(N2CCNC(C3CC3)C2)nc1]

Step 1: To a solution of tert-butyl 2-cyclopropylpiperazine-1-carboxylate (526 mg, 2.32 mmol, 1.1 eq) and 2-chloro-5-fluoro-pyrimidine (280 mg, 2.11 mmol, 1 eq) in NMP (6 mL) was added TEA (641 mg, 6.34 mmol, 3 eq). The mixture was stirred at 140° C. for 12 hours. LC-MS showed 2-chloro-5-fluoro-pyrimidine was consumed completely and desired mass was detected. The reaction mixture was partitioned between 20 mL of H₂O and 30 mL of EtOAc. The organic phase was separated, washed with 50 mL of brine, dried over Na₂SO₄, filtered and concentrated under reduced pressure to give the crude product. The residue was purified by column chromatography (SiO₂, petroleum ether/ethyl acetate=1/0 to 3/1) to give desired tert-butyl 2-cyclopropyl-4-(5-fluoropyrimidin-2-yl)piperazine-1-carboxylate (600 mg, 1.86 mmol, 88.1% yield) as a yellow solid. MS (ESI): mass calcd. For C₁₆H₂₃N₄FO₂ 322.18, m/z found 267.1 [M+H−56]⁺.

Step 2: To a solution of tert-butyl 2-cyclopropyl-4-(5-fluoropyrimidin-2-yl)piperazine-1-carboxylate (600 mg, 1.86 mmol, 1 eq) in HCl/EtOAc (4M, 6 mL). The mixture was stirred at 20° C. for 0.5 hour. LC-MS showed tert-butyl 2-cyclopropyl-4-(5-fluoropyrimidin-2-yl)piperazine-1-carboxylate was consumed completely and desired mass was detected. The reaction mixture was concentrated to give desired 2-(3-cyclopropylpiperazin-1-yl)-5-fluoro-pyrimidine (800 mg, crude) as a white solid. MS (ESI): mass calcd. For C₁₁H₁₅N₄F 222.13, m/z found 223.2 [M+H]⁺.

### Intermediate-L37: 4-chloro-3-(2-cyclopropyl-4-(5-fluoropyrimidin-2-yl)piperazin-1-yl)-1H-indazole

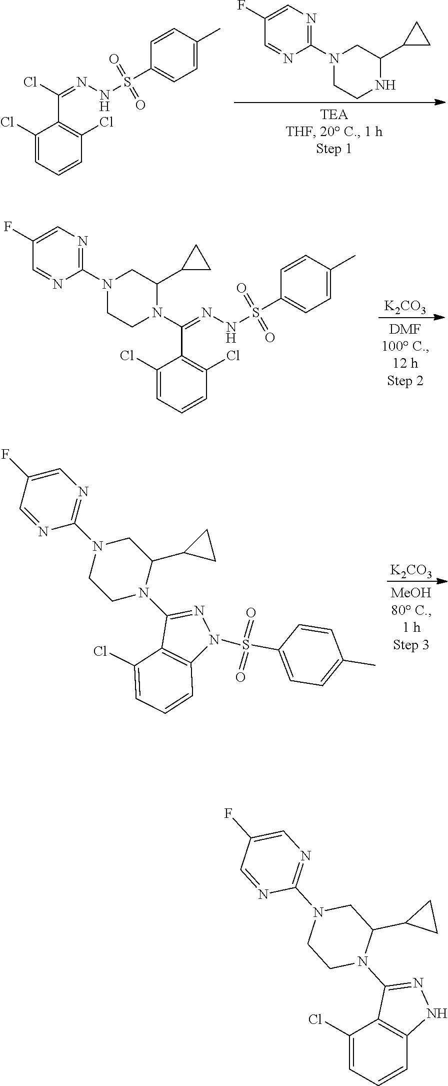

Step 1: To a solution of (1Z)-2,6-dichloro-N-(p-tolylsulfonyl)benzohydrazonoyl chloride (408 mg, 1.08 mmol, 1.2 eq) and 2-(3-cyclopropylpiperazin-1-yl)-5-fluoro-pyrimidine (200 mg, 900 μmol, 1 eq) in THF (6 mL) was added TEA (910 mg, 9.00 mmol, 1.25 mL, 10 eq). The mixture was stirred at 20° C. for 1 hour. LC-MS showed (1Z)-2,6-dichloro-N-(p-tolylsulfonyl)benzohydrazonoyl chloride was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N—[(Z)-[[2-cyclopropyl-4-(5-fluoropyrimidin-2-yl)piperazin-1-yl]-(2,6-dichlorophenyl)methylene]amino]-4-methyl-benzenesulfonamide (510 mg, crude) as a white solid. MS (ESI): mass calcd. For C₂₅H₂₅C₁₂N₆SO₂F 562.11, m/z found 563.2 [M+H]⁺.

Step 2: To a solution of N—[(Z)-[[2-cyclopropyl-4-(5-fluoropyrimidin-2-yl)piperazin-1-yl]-(2,6-dichlorophenyl)methylene]amino]-4-methyl-benzenesulfonamide (510 mg, 905 μmol, 1 eq) in DMF (6 mL) was added K₂CO₃ (1.25 g, 9.05 mmol, 10 eq). The mixture was stirred at 100° C. for 12 hours. LC-MS showed N—[(Z)-[[2-cyclopropyl-4-(5-fluoropyrimidin-2-yl)piperazin-1-yl]-(2,6-dichlorophenyl)methylene]amino]-4-methyl-benzenesulfonamide was consumed completely and desired mass was detected. The reaction mixture was partitioned between 50 mL of H₂O and 50 mL of EtOAc. The organic phase was separated, washed with 50 mL of brine, dried over Na₂SO₄, filtered and concentrated under reduced pressure to give desired 4-chloro-3-[2-cyclopropyl-4-(5-fluoropyrimidin-2-yl)piperazin-1-yl]-1-(p-tolylsulfonyl)indazole (490 mg, crude) as a black oil. MS (ESI): mass calcd. For C₂₅H₂₄N₆FClSO₂ 526.14, m/z found 527.2 [M+H]⁺.

Step 3: To a solution of 4-chloro-3-[2-cyclopropyl-4-(5-fluoropyrimidin-2-yl)piperazin-1-yl]-1-(p-tolylsulfonyl)indazole (490 mg, 930 μmol, 1 eq) in MeOH (5 mL) was added K₂CO₃ (643 mg, 4.65 mmol, 5 eq). The mixture was stirred at 80° C. for 1 hour. LC-MS showed 4-chloro-3-[2-cyclopropyl-4-(5-fluoropyrimidin-2-yl)piperazin-1-yl]-1-(p-tolylsulfonyl)indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether:ethyl acetate=2:1) to give desired 4-chloro-3-[2-cyclopropyl-4-(5-fluoropyrimidin-2-yl)piperazin-1-yl]-1H-indazole (25 mg, 67.1 μmol, 7.21% yield) as a white solid. MS (ESI): mass calcd. For C₁₈H₁₈ClN₆F 372.13, m/z found 373.1 [M+H]⁺.

### Intermediate-L38: 4-chloro-3-(2-cyclopropyl-4-(5-fluoropyrimidin-2-yl)piperazin-1-yl)-6-fluoro-1H-indazole

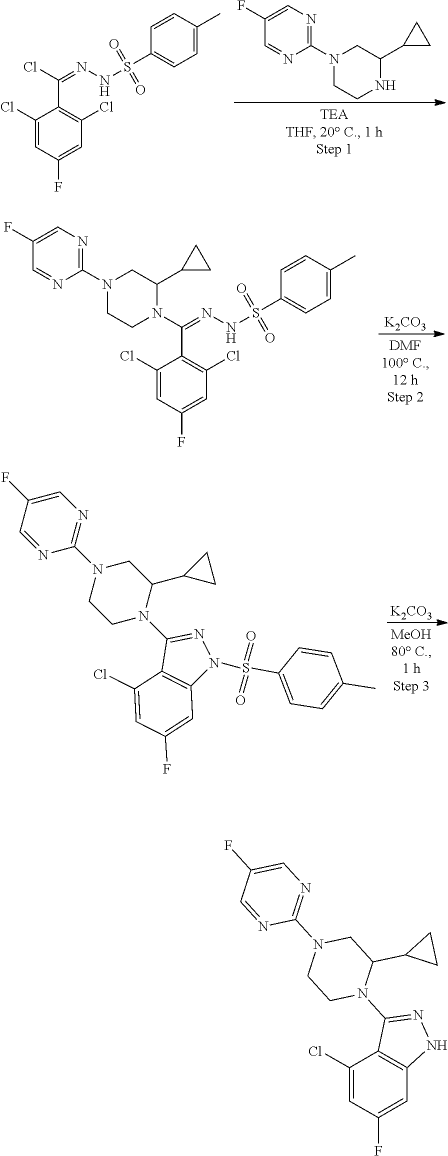

Step 1: To a solution of (1Z)-2,6-dichloro-4-fluoro-N-(p-tolylsulfonyl)benzohydrazonoyl chloride (367 mg, 928 μmol, 1.2 eq) and 2-(3-cyclopropylpiperazin-1-yl)-5-fluoro-pyrimidine (200 mg, 773 μmol, 1 eq, HCl) in THF (6 mL) was added TEA (782 mg, 7.73 mmol, 1.08 mL, 10 eq). The mixture was stirred at 20° C. for 1 hour. LC-MS showed (1Z)-2,6-dichloro-4-fluoro-N-(p-tolylsulfonyl)benzohydrazonoyl chloride was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N—[(Z)-[[2-cyclopropyl-4-(5-fluoropyrimidin-2-yl)piperazin-1-yl]-(2,6-dichloro-4-fluoro-phenyl)methylene]amino]-4-methyl-benzenesulfonamide (450 mg, crude) as a white solid. MS (ESI): mass calcd. For C₂₅H₂₄C₁₂N₆SO₂F₂ 580.1, m/z found 581.2 [M+H]⁺.

Step 2: To a solution of N—[(Z)-[[2-cyclopropyl-4-(5-fluoropyrimidin-2-yl)piperazin-1-yl]-(2,6-dichloro-4-fluoro-phenyl)methylene]amino]-4-methyl-benzenesulfonamide (450 mg, 774 μmol, 1 eq) in DMF (6 mL) was added K₂CO₃ (1.07 g, 7.74 mmol, 10 eq). The mixture was stirred at 100° C. for 12 hours. LC-MS showed N—[(Z)-[[2-cyclopropyl-4-(5-fluoropyrimidin-2-yl)piperazin-1-yl]-(2,6-dichloro-4-fluoro-phenyl)methylene]amino]-4-methyl-benzenesulfonamide was consumed completely and desired mass was detected. The reaction mixture was partitioned between 50 mL of H₂O and 50 mL of EtOAc. The organic phase was separated, washed with 50 mL of brine, dried over Na₂SO₄, filtered and concentrated under reduced pressure to give desired 4-chloro-3-[2-cyclopropyl-4-(5-fluoropyrimidin-2-yl)piperazin-1-yl]-6-fluoro-1-(p-tolylsulfonyl)indazole (440 mg, crude) as a black oil. MS (ESI): mass calcd. For C₂₅H₂₃F₂ClN₆SO₂ 544.13, m/z found 545.1 [M+H]⁺.

Step 3: To a solution of 4-chloro-3-[2-cyclopropyl-4-(5-fluoropyrimidin-2-yl)piperazin-1-yl]-6-fluoro-1-(p-tolylsulfonyl)indazole (440 mg, 807 μmol, 1 eq) in MeOH (5 mL) was added K₂CO₃ (558 mg, 4.04 mmol, 5 eq). The mixture was stirred at 80° C. for 1 hour. LC-MS showed 4-chloro-3-[2-cyclopropyl-4-(5-fluoropyrimidin-2-yl)piperazin-1-yl]-6-fluoro-1-(p-tolylsulfonyl)indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether:ethyl acetate=2:1) to give desired 4-chloro-3-[2-cyclopropyl-4-(5-fluoropyrimidin-2-yl)piperazin-1-yl]-6-fluoro-1H-indazole (17 mg, 43.5 μmol, 5.39% yield) as a white solid. MS (ESI): mass calcd. For C₁₈H₁₇ClN₆F₂ 390.12, m/z found 391.1 [M+H]⁺.

### Intermediate-L39: 1-[4-chloro-1-(p-tolylsulfonyl) indazol-3-yl]-2-(trifluoromethyl) piperidin-4-one

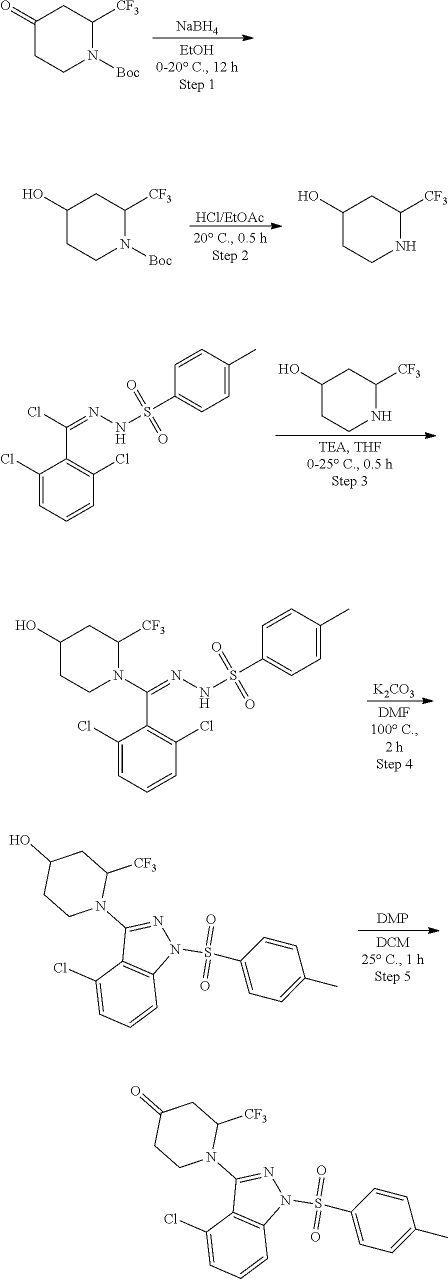

Step 1: To a solution of tert-butyl 4-oxo-2-(trifluoromethyl) piperidine-1-carboxylate (1.14 g, 4.27 mmol, 1 eq) in EtOH (12 mL) was added NaBH₄ (323 mg, 8.53 mmol, 2 eq) at 0° C. The mixture was stirred at 20° C. for 12 hours. TLC (petroleum ether/ethyl acetate=3/1) indicated tert-butyl 4-oxo-2-(trifluoromethyl) piperidine-1-carboxylate was consumed completely and one new spot was formed. The reaction was clean according to TLC. The residue was diluted with HCl (1N, 8 mL) and the resulting mixture was allowed to return to room temperature. The MeOH was evaporated in vacuo. The reaction mixture was added to water (20 mL), extracted with DCM (10 mL*3). The combined organic layers were washed with brine (20 mL) and dried over Na₂SO₄, filtered and concentrated under reduced pressure to give desired tert-butyl 4-hydroxy-2-(trifluoromethyl)piperidine-1-carboxylate (1.2 g, crude) as a white solid.

Step 2: A mixture of tert-butyl 4-hydroxy-2-(trifluoromethyl) piperidine-1-carboxylate (1.2 g, 4.46 mmol, 1.0 eq) in HCl/EtOAc (4M, 20 mL) was stirred at 20° C. for 0.5 hour. TLC (petroleum ether/ethyl acetate=3/1) indicated tert-butyl 4-hydroxy-2-(trifluoromethyl) piperidine-1-carboxylate was consumed completely and one new spot was formed. The reaction was clean according to TLC. The reaction mixture was concentrated to give desired 2-(trifluoromethyl) piperidin-4-ol (1.1 g, crude, HCl) as a yellow solid.

Step 3: To a solution of 2-(trifluoromethyl) piperidin-4-ol (220 mg, 1.07 mmol, 1 eq, HCl) in THF (3 mL) was added dropwise TEA (1.08 g, 10.7 mmol, 1.49 mL, 10 eq) at 25° C. After addition, the mixture was stirred at this temperature for 10 mins, and then (1Z)-2,6-dichloro-N-(p-tolylsulfonyl)benzohydrazonoyl chloride (445 mg, 1.18 mmol, 1.1 eq) in THF (3 mL) was added dropwise at 0° C. The resulting mixture was stirred at 25° C. for 20 mins. LC-MS showed (1Z)-2, 6-dichloro-N-(p-tolylsulfonyl) benzohydrazonoyl chloride was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(E)-[(2,6-dichlorophenyl)-[4-hydroxy-2-(trifluoromethyl)-1-piperidyl]methylene]amino]-4-methyl-benzenesulfonamide (600 mg, crude) as a yellow solid. MS (ESI): mass calcd. For C₂₀H₂₀C₁₂F₃N₃O₃S 509.06, mass found 510.0 [M+H]⁺.

Step 4: To a solution of N—[(E)-[(2, 6-dichlorophenyl)-[4-hydroxy-2-(trifluoromethyl)-1-piperidyl]methylene]amino]-4-methyl-benzenesulfonamide (550 mg, 1.08 mmol, 1 eq) in DMF (2 mL) was added K₂CO₃ (596 mg, 4.31 mmol, 4 eq). The mixture was stirred at 100° C. for 2 hours. LC-MS showed N—[(E)-[(2, 6-dichlorophenyl)-[4-hydroxy-2-(trifluoromethyl)-1-piperidyl]methylene]amino]-4-methyl-benzenesulfonamide was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give desired 1-[4-chloro-1-(p-tolylsulfonyl)indazol-3-yl]-2-(trifluoromethyl)piperidin-4-ol (510 mg, crude) as an orange oil. MS (ESI): mass calcd. For C₂₀H₁₉ClF₃N₃O₃S 473.08, mass found 473.9 [M+H]⁺.

Step 5: To a solution of 1-[4-chloro-1-(p-tolylsulfonyl) indazol-3-yl]-2-(trifluoromethyl) piperidin-4-ol (510 mg, 1.08 mmol, 1 eq) in DCM (10 mL) was added DMP (685 mg, 1.61 mmol, 1.5 eq). The mixture was stirred at 25° C. for 1 hour. LC-MS showed 1-[4-chloro-1-(p-tolylsulfonyl) indazol-3-yl]-2-(trifluoromethyl) piperidin-4-ol was consumed completely and desired mass was detected. The reaction mixture was added to sat.aq.NaHCO₃ (20 mL), extracted with DCM (10 mL*3). The combined organic layers were washed with brine 20 mL and dried over Na₂SO₄, filtered and concentrated under reduced pressure to give a residue. The residue was purified by prep-TLC (Silica gel, petroleum ether/ethyl acetate=3/1) to give desired 1-[4-chloro-1-(p-tolylsulfonyl) indazol-3-yl]-2-(trifluoromethyl) piperidin-4-one (110 mg, crude) as a yellow solid. MS (ESI): mass calcd. For C₂₀H₁₇ClF₃N₃O₃S 471.06, mass found 472.0 [M+H]⁺.

### Intermediate-L40: 4-chloro-1-(4-isopropylsulfonylphenyl) sulfonyl-3-[2-(trifluoromethyl) piperazin-1-yl]indazole

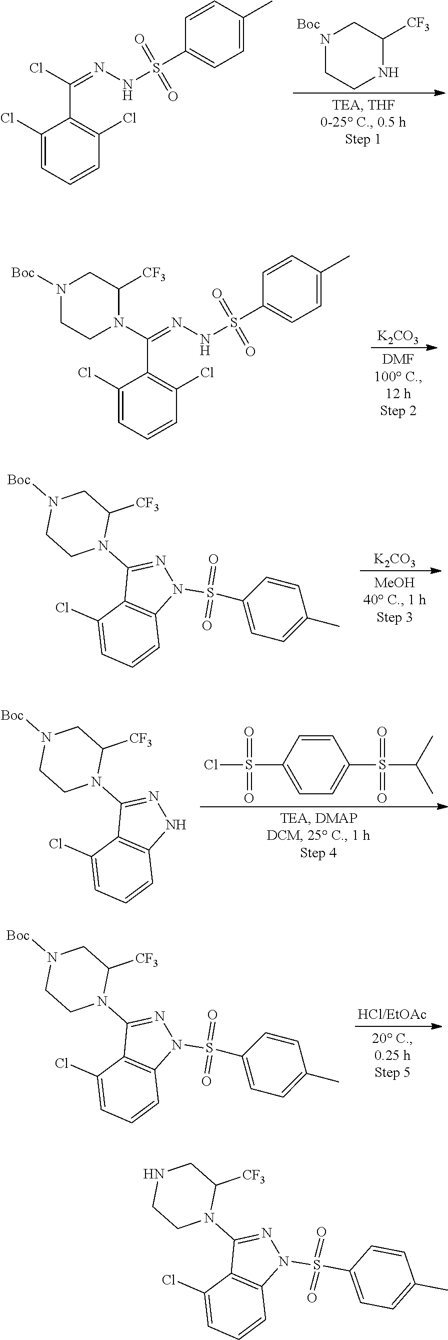

Step 1: To a solution of (1E)-2,6-dichloro-N-(p-tolylsulfonyl)benzohydrazonoyl chloride (446 mg, 1.18 mmol, 1.5 eq) and tert-butyl 3-(trifluoromethyl)piperazine-1-carboxylate (200 mg, 787 μmol, 1 eq) in THF (7 mL) was added TEA (398 mg, 3.93 mmol, 547 μL, 5 eq) at 0° C. The mixture was stirred at 25° C. for 1 hour. LC-MS showed tert-butyl 3-(trifluoromethyl) piperazine-1-carboxylate was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired tert-butyl 4-[(E)-C-(2,6-dichlorophenyl)-N-(p-tolylsulfonylamino)carbonimidoyl]-3-(trifluoromethyl)piperazine-1-carboxylate (640 mg, crude) as a yellow solid. MS (ESI): mass calcd. For C₂₄H₂₇Cl₂F₃N₄O₄S 594.1 mass found 595.1 [M+H]⁺

Step 2: To a solution of tert-butyl 4-[(E)-C-(2, 6-dichlorophenyl)-N-(p-tolylsulfonylamino) carbonimidoyl]-3-(trifluoromethyl) piperazine-1-carboxylate (640 mg, 1.07 mmol, 1 eq) in DMF (8 mL) was added K₂CO₃ (743 mg, 5.37 mmol, 5 eq). The mixture was stirred at 100° C. for 12 hours. LC-MS showed tert-butyl 4-[(E)-C-(2, 6-dichlorophenyl)-N-(p-tolylsulfonylamino) carbonimidoyl]-3-(trifluoromethyl) piperazine-1-carboxylate was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired tert-butyl 4-[4-chloro-1-(p-tolylsulfonyl)indazol-3-yl]-3-(trifluoromethyl)piperazine-1-carboxylate (600 mg, crude) as a yellow oil. MS (ESI): mass calcd. For C₂₄H₂₆ClF₃N₄O₄S 558.1 mass found 559.1 [M+H]⁺.

Step 3: To a solution of tert-butyl 4-[4-chloro-1-(p-tolylsulfonyl) indazol-3-yl]-3-(trifluoromethyl) piperazine-1-carboxylate (600 mg, 1.07 mmol, 1 eq) in MeOH (7 mL) was added K₂CO₃ (742 mg, 5.37 mmol, 5 eq). The mixture was stirred at 40° C. for 1 hour. LC-MS showed tert-butyl 4-[4-chloro-1-(p-tolylsulfonyl) indazol-3-yl]-3-(trifluoromethyl) piperazine-1-carboxylate was consumed completely and desired mass was detected. The reaction mixture was partitioned between 20 mL of H₂O and 30 mL of EtOAc. The organic phase was separated, washed with 50 mL of brine, dried over Na₂SO₄, filtered and concentrated under reduced pressure to give the crude product. The residue was purified by prep-TLC (Silica gel, petroleum ether/ethyl acetate=3/1) to give desired tert-butyl 4-(4-chloro-1H-indazol-3-yl)-3-(trifluoromethyl) piperazine-1-carboxylate (130 mg, 321 μmol, 29.9% yield) as a yellow solid. MS (ESI): mass calcd. For C₁₇H₂₀ClF₃N₄O₂ 404.1, mass found 405.1 [M+H]⁺.

Step 4: To a solution of tert-butyl 4-(4-chloro-1H-indazol-3-yl)-3-(trifluoromethyl)piperazine-1-carboxylate (130 mg, 321 μmol, 1 eq) and 4-isopropylsulfonylbenzenesulfonyl chloride (136 mg, 482 μmol, 1.5 eq) in DCM (2 mL) was added TEA (65.0 mg, 643 μmol, 89.4 μL, 2 eq) and DMAP (3.92 mg, 32.1 μmol, 0.1 eq). The mixture was stirred at 25° C. for 1 hour. LC-MS showed tert-butyl 4-(4-chloro-1H-indazol-3-yl)-3-(trifluoromethyl) piperazine-1-carboxylate was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-TLC (Silica gel, petroleum ether/ethyl acetate=5/1) to give desired tert-butyl 4-[4-chloro-1-(4-isopropylsulfonylphenyl) sulfonyl-indazol-3-yl]-3-(trifluoromethyl) piperazine-1-carboxylate (75 mg, 115 μmol, 35.87% yield) as a yellow solid. MS (ESI): mass calcd. For C₂₄H₂₆ClF₃N₄O₄S 558.1, mass found 559.1 [M+H]⁺.

Step 5: To a solution of tert-butyl 4-[4-chloro-1-(4-isopropylsulfonylphenyl) sulfonyl-indazol-3-yl]-3-(trifluoromethyl) piperazine-1-carboxylate (75 mg, 115 μmol, 1 eq) in HCl/EtOAc (4 M, 4 mL, 139 eq). The mixture was stirred at 25° C. for 0.25 hour. LC-MS showed tert-butyl 4-[4-chloro-1-(4-isopropylsulfonylphenyl) sulfonyl-indazol-3-yl]-3-(trifluoromethyl) piperazine-1-carboxylate was consumed completely and desired mass was detected. The reaction mixture was filtered and the filter liquor was concentrated under reduced pressure to give desired 4-chloro-1-(4-isopropylsulfonylphenyl) sulfonyl-3-[2-(trifluoromethyl) piperazin-1-yl]indazole (76 mg, crude) as a white solid. MS (ESI): mass calcd. For C₁₉H₁₈ClF₃N₄O₂S 458.1, mass found 459.1 [M+H]⁺.

### Intermediate-L41: 4-chloro-3-(4, 7-diazaspiro [2.5]octan-4-yl)-1-(4-isopropylsulfonylphenyl) sulfonyl-indazole

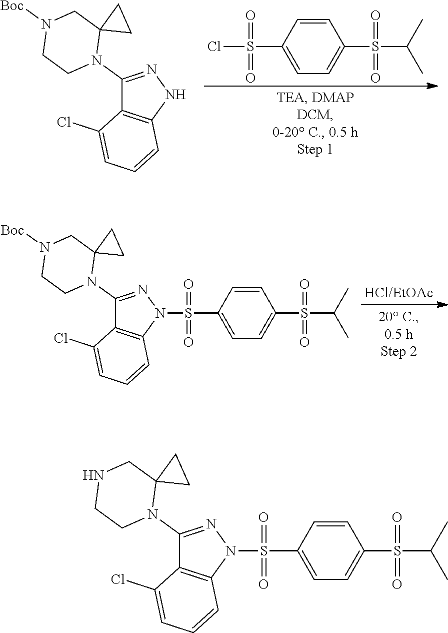 [SMILES: CC(C)(C)OC(=O)N1CCN(c2n[nH]c3cccc(Cl)c23)C2(CC2)C1 ; CC(C)S(=O)(=O)c1ccc(S(=O)(=O)Cl)cc1 ; CC(C)S(=O)(=O)c1ccc(S(=O)(=O)n2nc(N3CCN(C(=O)OC(C)(C)C)CC34CC4)c3c(Cl)cccc32)cc1 ; CC(C)S(=O)(=O)c1ccc(S(=O)(=O)n2nc(N3CCNCC34CC4)c3c(Cl)cccc32)cc1]

Step 1: To a solution of tert-butyl 4-(4-chloro-1H-indazol-3-yl)-4, 7-diazaspiro [2.5]octane-7-carboxylate (100 mg, 276 μmol, 1 eq) and 4-isopropylsulfonylbenzenesulfonyl chloride (117 mg, 413 μmol, 1.5 eq) in DCM (2 mL) was added TEA (55.8 mg, 551 μmol, 76.7 μL, 2 eq) and DMAP (3.37 mg, 27.6 μmol, 0.1 eq) at 0° C. The mixture was stirred at 20° C. for 0.5 hour. LC-MS showed tert-butyl 4-(4-chloro-1H-indazol-3-yl)-4, 7-diazaspiro [2.5]octane-7-carboxylate was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (Silica gel, petroleum ether/ethyl acetate=2/1) to give desired tert-butyl 4-[4-chloro-1-(4-isopropylsulfonylphenyl) sulfonyl-indazol-3-yl]-4, 7-diazaspiro [2.5]octane-7-carboxylate (160 mg, crude) as a white solid. MS (ESI): mass calcd. For C₂₇H₃₃ClN₄O₆S₂ 608.15, mass found 556.9 [M+H−56]⁺.

Step 2: A mixture of tert-butyl 4-[4-chloro-1-(4-isopropylsulfonylphenyl) sulfonyl-indazol-3-yl]-4, 7-diazaspiro [2.5]octane-7-carboxylate (160 mg, 263 μmol, 1 eq) in HCl/EtOAc (4M, 5 mL) was stirred at 20° C. for 0.5 hour. LC-MS showed tert-butyl 4-[4-chloro-1-(4-isopropylsulfonylphenyl) sulfonyl-indazol-3-yl]-4, 7-diazaspiro [2.5]octane-7-carboxylate was consumed completely and desired mass was detected. The reaction mixture was concentrated to give desired 4-chloro-3-(4, 7-diazaspiro [2.5]octan-4-yl)-1-(4-isopropylsulfonylphenyl) sulfonyl-indazole (150 mg, crude, HCl salt) as a yellow solid. MS (ESI): mass calcd. For C₂₂H₂₅ClN₄O₄S₂ 508.10, mass found 509.0 [M+H]⁺.

### Intermediate-L42: 7-pyrimidin-2-yl-4, 7-diazaspiro [2.5]octane

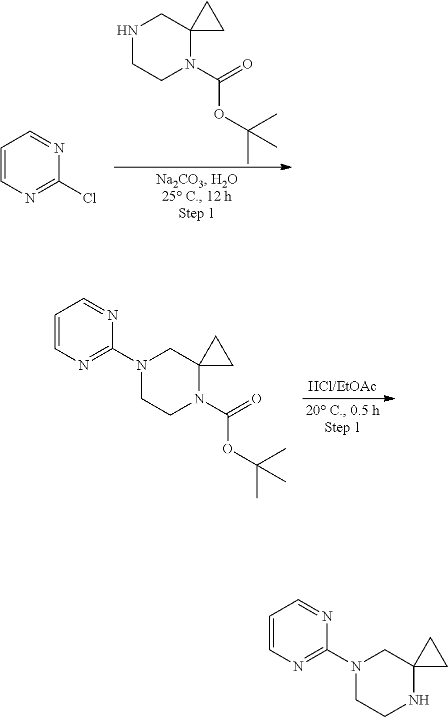 [SMILES: Clc1ncccn1 ; CC(C)(C)OC(=O)N1CCNCC12CC2 ; CC(C)(C)OC(=O)N1CCN(c2ncccn2)CC12CC2 ; c1cnc(N2CCNC3(CC3)C2)nc1]

Step 1: To a solution of Na₂CO₃ (925 mg, 8.73 mmol, 1.1 eq) in H₂O (10 mL) was added 2-chloropyrimidine (1 g, 8.73 mmol, 1.1 eq) and tert-butyl 4, 7-diazaspiro [2.5]octane-4-carboxylate (1.69 g, 7.94 mmol, 1 eq). The mixture was stirred at 25° C. for 12 hours. LC-MS showed 2-chloropyrimidine was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give desired tert-butyl 7-pyrimidin-2-yl-4, 7-diazaspiro [2.5]octane-4-carboxylate (2.5 g, crude) as an orange oil. MS (ESI): mass calcd. For C₁₅H₂₂N₄O₂ 290.17 mass found 291.2 [M+H]⁺.

Step 2: A mixture of tert-butyl 7-pyrimidin-2-yl-4, 7-diazaspiro [2.5]octane-4-carboxylate (2.5 g, 8.61 mmol, 1 eq) in HCl/EtOAc (4M, 30 mL) was stirred at 20° C. for 0.5 hour. LC-MS showed tert-butyl 7-pyrimidin-2-yl-4, 7-diazaspiro [2.5]octane-4-carboxylate was consumed completely and desired mass was detected. The reaction mixture was concentrated to give desired 7-pyrimidin-2-yl-4, 7-diazaspiro [2.5]octane (2 g, crude, HCl) as a yellow solid. MS (ESI): mass calcd. For C₁₀H₁₄N₄ 190.12 mass found 191.1 [M+H]⁺.

### Intermediate-L43: 4-chloro-7-fluoro-3-[7-(5-fluoropyrimidin-2-yl)-4, 7-diazaspiro [2.5]octan-4-yl]-1H-indazole

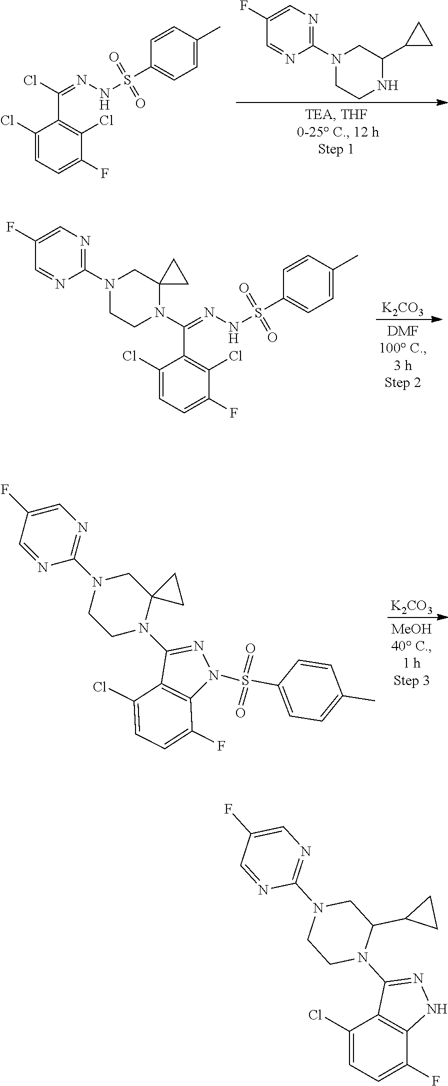

Step 1: To a solution of (1E)-2, 6-dichloro-3-fluoro-N-(p-tolylsulfonyl) benzohydrazonoyl chloride (1.14 g, 2.88 mmol, 1.5 eq) and 7-(5-fluoropyrimidin-2-yl)-4, 7-diazaspiro [2.5]octane (400 mg, 1.92 mmol, 1 eq) in THF (5 mL) was added TEA (1.94 g, 19.2 mmol, 2.67 mL, 10 eq) at 0° C. The mixture was stirred at 25° C. for 12 hours. LC-MS showed 7-(5-fluoropyrimidin-2-yl)-4, 7-diazaspiro [2.5]octane was consumed completely and one main peak with desired mass was detected. The reaction mixture was added to water (20 mL) and extracted with EtOAc (20 mL*3). The combined organic layers were washed with brine (20 mL) and dried over Na₂SO₄, filtered and concentrated under reduced pressure to give desired N—[(E)-[(2, 6-dichloro-3-fluoro-phenyl)-[7-(5-fluoropyrimidin-2-yl)-4, 7-diazaspiro [2.5]octan-4-yl]methylene]amino]-4-methyl-benzenesulfonamide (1.17 g, crude) as a white solid. MS (ESI): mass calcd. For C₂₄H₂₂N₆F₂SO₂Cl₂ 566.09, mass found 567.1[M+H]⁺.

Step 2: To a solution of N—[(E)-[(2, 6-dichloro-3-fluoro-phenyl)-[7-(5-fluoropyrimidin-2-yl)-4, 7-diazaspiro [2.5]octan-4-yl]methylene]amino]-4-methyl-benzenesulfonamide (1 g, 1.76 mmol, 1 eq) in DMF (10 mL) was added K₂CO₃ (974 mg, 7.05 mmol, 4 eq). The mixture was stirred at 100° C. for 3 hours. LC-MS showed N—[(E)-[(2, 6-dichloro-3-fluoro-phenyl)-[7-(5-fluoropyrimidin-2-yl)-4, 7-diazaspiro [2.5]octan-4-yl]methylene]amino]-4-methyl-benzenesulfonamide was consumed completely and desired mass was detected. The reaction mixture was added to water (30 mL) and extracted with MTBE (30 mL*3). The combined organic layers were washed with brine (30 mL) and dried over Na₂SO₄, filtered and concentrated under reduced pressure to give desired 4-chloro-7-fluoro-3-[7-(5-fluoropyrimidin-2-yl)-4,7-diazaspiro[2.5]octan-4-yl]-1-(p-tolylsulfonyl)indazole (2.5 g, crude) as a white solid. MS (ESI): mass calcd. For C₂₄H₂₁ClF₂N₆SO₂ 530.11, mass found 531.2[M+H]⁺.

Step 3: To a solution of 4-chloro-7-fluoro-3-[7-(5-fluoropyrimidin-2-yl)-4, 7-diazaspiro [2.5]octan-4-yl]-1-(p-tolylsulfonyl) indazole (2.5 g, 4.71 mmol, 1 eq) in MeOH (5 mL) was added K₂CO₃ (3.25 g, 23.5 mmol, 5 eq). The mixture was stirred at 40° C. for 1 hour. LC-MS showed 4-chloro-7-fluoro-3-[7-(5-fluoropyrimidin-2-yl)-4, 7-diazaspiro [2.5]octan-4-yl]-1-(p-tolylsulfonyl) indazole was consumed completely and one main peak with desired mass was detected. The reaction mixture was added to water (30 mL), extracted with EtOAc (30 mL*3). The combined organic layers were washed with brine (30 mL) and dried over Na₂SO₄, filtered and concentrated under reduced pressure to give a residue. The residue was purified by prep-TLC (Silica gel, petroleum ether/ethyl acetate=3/1) to give desired 4-chloro-7-fluoro-3-[7-(5-fluoropyrimidin-2-yl)-4, 7-diazaspiro [2.5]octan-4-yl]-1H-indazole (230 mg, 610 μmol, 13% yield) as a white solid. MS (ESI): mass calcd. For C₁₇H₁₅ClF₂N₆ 376.1, mass found 377.0[M+H]⁺.

### Intermediate-L44: 4-chloro-3-(4, 7-diazaspiro [2.5]octan-4-yl)-1-[4-(1, 1-difluoroethyl) phenyl]sulfonyl-indazole

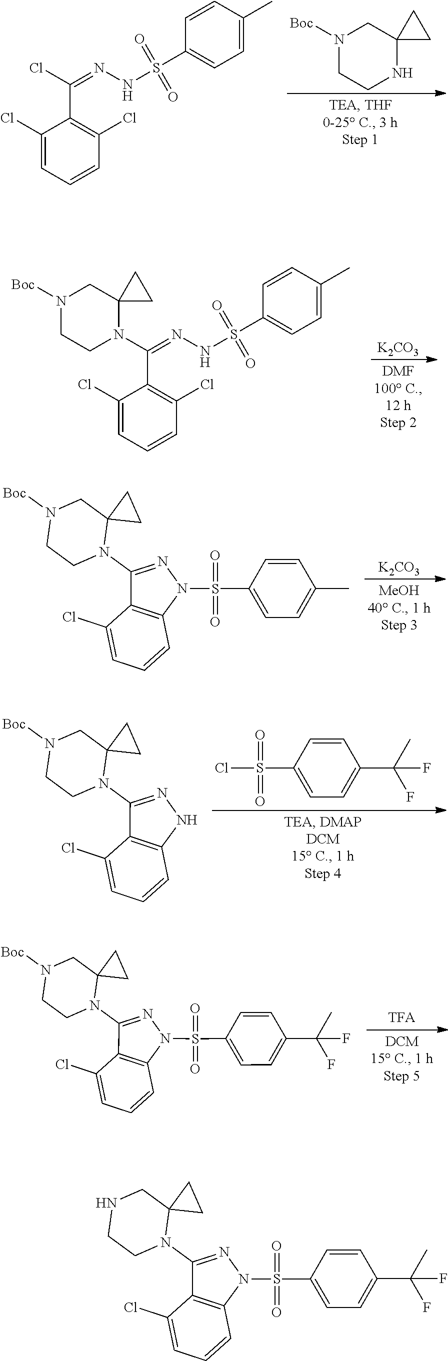

Step 1: To a solution of tert-butyl 4,7-diazaspiro[2.5]octane-7-carboxylate (300 mg, 1.41 mmol, 1 eq) and (1E)-2,6-dichloro-N-(p-tolylsulfonyl)benzohydrazonoyl chloride (800 mg, 2.12 mmol, 1.5 eq) in THF (3 mL) was added TEA (715 mg, 7.07 mmol, 983 μL, 5 eq) at 0° C. The mixture was stirred at 15° C. for 3 hours. LC-MS showed tert-butyl 4, 7-diazaspiro [2.5]octane-7-carboxylate was consumed completely and desired mass was detected. The reaction mixture was added to H₂O (30 mL), extracted with EtOAc (30 mL*3). The combined organic layers were washed with brine (30 mL) and dried over Na₂SO₄, filtered and concentrated under reduced pressure to give desired tert-butyl 4-[(E)-C-(2,6-dichlorophenyl)-N-(p-tolylsulfonylamino)carbonimidoyl]-4,7-diazaspiro[2.5]octane-7-carboxylate (1.1 g, crude) as a yellow solid. MS (ESI): mass calcd. For C₂₅H₃₀N₄SO₄C₁₂ 552.1, m/z found 553.1 [M+H]⁺.

Step 2: To a solution of tert-butyl 4-[(E)-C-(2, 6-dichlorophenyl)-N-(p-tolylsulfonylamino) carbonimidoyl]-4, 7-diazaspiro [2.5]octane-7-carboxylate (1.1 g, 1.99 mmol, 1 eq) in DMF (20 mL) was added K₂CO₃ (1.10 g, 7.95 mmol, 4 eq). The mixture was stirred at 100° C. for 12 hours. LCMS showed tert-butyl 4-[(E)-C-(2, 6-dichlorophenyl)-N-(p-tolylsulfonylamino) carbonimidoyl]-4, 7-diazaspiro [2.5]octane-7-carboxylate was consumed completely and desired mass was detected. The reaction mixture was added to H₂O (30 mL), extracted with EtOAc (30 mL*3). The combined organic layers were washed with brine (30 mL) and dried over Na₂SO₄, filtered and concentrated under reduced pressure to give desired tert-butyl 4-[4-chloro-1-(p-tolylsulfonyl)indazol-3-yl]-4,7-diazaspiro[2.5]octane-7-carboxylate (890 mg, crude) as a yellow oil. MS (ESI): mass calcd. For C₂₅H₂₉N₄SO₄Cl 516.16, m/z found 461.1 [M+H−56]⁺.

Step 3: To a solution of tert-butyl 4-[4-chloro-1-(p-tolylsulfonyl) indazol-3-yl]-4, 7-diazaspiro [2.5]octane-7-carboxylate (890 mg, 1.72 mmol, 1 eq) in MeOH (20 mL) was added K₂CO₃ (1.19 g, 8.61 mmol, 5 eq). The mixture was stirred at 40° C. for 1 hour. LC-MS showed tert-butyl 4-[4-chloro-1-(p-tolylsulfonyl) indazol-3-yl]-4, 7-diazaspiro [2.5]octane-7-carboxylate was consumed completely and desired mass was detected. The reaction mixture was added to H₂O (30 mL), extracted with EtOAc (30 mL*3). The combined organic layers were washed with brine (30 mL) and dried over Na₂SO₄, filtered and concentrated under reduced pressure to give a residue. The residue was purified by prep-TLC (SiO₂, petroleum ether/Ethyl acetate=3/1) to give desired tert-butyl 4-(4-chloro-1H-indazol-3-yl)-4, 7-diazaspiro [2.5]octane-7-carboxylate (350 mg, 964 μmol, 56.0% yield) as a yellow solid. MS (ESI): mass calcd. For C₁₈H₂₃N₄ClO₂ 362.15, m/z found 363.0 [M+H]⁺.

Step 4: To a solution of 4-(1, 1-difluoroethyl)benzenesulfonyl chloride (265 mg, 1.10 mmol, 2 eq) and tert-butyl 4-(4-chloro-1H-indazol-3-yl)-4,7-diazaspiro[2.5]octane-7-carboxylate (200 mg, 551 μmol, 1 eq) in DCM (2 mL) was added TEA (111 mg, 1.10 mmol, 153 μL, 2 eq) and DMAP (6.73 mg, 55.1 μmol, 0.1 eq). The mixture was stirred at 15° C. for 1 hour. LC-MS showed tert-butyl 4-(4-chloro-1H-indazol-3-yl)-4, 7-diazaspiro [2.5]octane-7-carboxylate was consumed completely desired mass was detected. The reaction mixture was added to H₂O (30 mL), extracted with EtOAc (30 mL*3). The combined organic layers were washed with brine (20 mL) and dried over Na₂SO₄, filtered and concentrated under reduced pressure to give a residue. The residue was purified by prep-TLC (SiO₂, petroleum ether/Ethyl acetate=3/1) to give desired tert-butyl 4-[4-chloro-1-[4-(1, 1-difluoroethyl) phenyl]sulfonyl-indazol-3-yl]-4, 7-diazaspiro [2.5]octane-7-carboxylate (270 mg, 476 μmol, 86.39% yield) as a white solid. MS (ESI): mass calcd. For C₂₆H₂₉N₄F₂SO₄Cl 566.16 m/z found 511.1 [M+H−56]⁺.

Step 5: To a solution of tert-butyl 4-[4-chloro-1-[4-(1, 1-difluoroethyl) phenyl]sulfonyl-indazol-3-yl]-4, 7-diazaspiro [2.5]octane-7-carboxylate (270 mg, 476 μmol, 1 eq) in DCM (6 mL) was added TFA (2 mL). The mixture was stirred at 15° C. for 1 hour. LCMS showed tert-butyl 4-[4-chloro-1-[4-(1, 1-difluoroethyl) phenyl]sulfonyl-indazol-3-yl]-4, 7-diazaspiro [2.5]octane-7-carboxylate was consumed completely and desired mass was detected. The reaction mixture was concentrated to give desired 4-chloro-3-(4, 7-diazaspiro [2.5]octan-4-yl)-1-[4-(1, 1-difluoroethyl) phenyl]sulfonyl-indazole (350 mg, crude, TFA) as a light yellow solid. MS (ESI): mass calcd. For C₂₃H₂₂N₄F₅SO₄Cl 466.1 m/z found 467.1 [M+H]⁺.

### Intermediate-L45: 4-chloro-1-(4-isopropylsulfonylphenyl) sulfonyl-3-[2-(trifluoromethyl) piperazin-1-yl]indazole

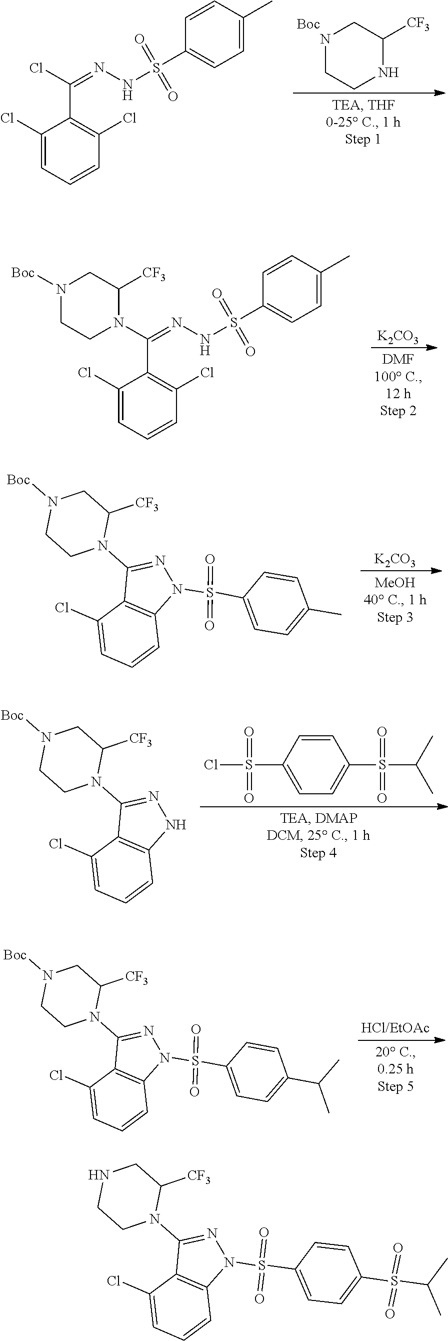

Step 1: To a solution of (1E)-2,6-dichloro-N-(p-tolylsulfonyl)benzohydrazonoyl chloride (446 mg, 1.18 mmol, 1.5 eq) and tert-butyl 3-(trifluoromethyl)piperazine-1-carboxylate (200 mg, 787 μmol, 1 eq) in THF (7 mL) was added TEA (398 mg, 3.93 mmol, 547 μL, 5 eq) at 0° C. The mixture was stirred at 25° C. for 1 hour. LC-MS showed tert-butyl 3-(trifluoromethyl) piperazine-1-carboxylate was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired tert-butyl 4-[(E)-C-(2,6-dichlorophenyl)-N-(p-tolylsulfonylamino)carbonimidoyl]-3-(trifluoromethyl)piperazine-1-carboxylate (640 mg, crude) as a yellow solid. MS (ESI): mass calcd. For C₂₄H₂₇Cl₂F₃N₄O₄S 594.1 mass found 595.1 [M+H]⁺

Step 2: To a solution of tert-butyl 4-[(E)-C-(2, 6-dichlorophenyl)-N-(p-tolylsulfonylamino) carbonimidoyl]-3-(trifluoromethyl) piperazine-1-carboxylate (640 mg, 1.07 mmol, 1 eq) in DMF (8 mL) was added K₂CO₃ (743 mg, 5.37 mmol, 5 eq). The mixture was stirred at 100° C. for 12 hours. LC-MS showed tert-butyl 4-[(E)-C-(2, 6-dichlorophenyl)-N-(p-tolylsulfonylamino) carbonimidoyl]-3-(trifluoromethyl) piperazine-1-carboxylate was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired tert-butyl 4-[4-chloro-1-(p-tolylsulfonyl)indazol-3-yl]-3-(trifluoromethyl)piperazine-1-carboxylate (600 mg, crude) as a yellow oil. MS (ESI): mass calcd. For C₂₄H₂₆ClF₃N₄O₄S 558.1 mass found 559.1 [M+H]⁺.

Step 3: To a solution of tert-butyl 4-[4-chloro-1-(p-tolylsulfonyl) indazol-3-yl]-3-(trifluoromethyl) piperazine-1-carboxylate (600 mg, 1.07 mmol, 1 eq) in MeOH (7 mL) was added K₂CO₃ (742 mg, 5.37 mmol, 5 eq). The mixture was stirred at 40° C. for 1 hour. LC-MS showed tert-butyl 4-[4-chloro-1-(p-tolylsulfonyl) indazol-3-yl]-3-(trifluoromethyl) piperazine-1-carboxylate was consumed completely and desired mass was detected. The reaction mixture was partitioned between 20 mL of H₂O and 30 mL of EtOAc. The organic phase was separated, washed with 50 mL of brine, dried over Na₂SO₄, filtered and concentrated under reduced pressure to give the crude product. The residue was purified by prep-TLC (Silica gel, petroleum ether/ethyl acetate=3/1) to give desired tert-butyl 4-(4-chloro-1H-indazol-3-yl)-3-(trifluoromethyl) piperazine-1-carboxylate (130 mg, 321 μmol, 29.9% yield) as a yellow solid. MS (ESI): mass calcd. For C₁₇H₂₀ClF₃N₄O₂ 404.1, mass found 405.1 [M+H]⁺.

Step 4: To a solution of tert-butyl 4-(4-chloro-1H-indazol-3-yl)-3-(trifluoromethyl)piperazine-1-carboxylate (130 mg, 321 μmol, 1 eq) and 4-isopropylsulfonylbenzenesulfonyl chloride (136 mg, 482 μmol, 1.5 eq) in DCM (2 mL) was added TEA (65.0 mg, 643 μmol, 89.4 μL, 2 eq) and DMAP (3.92 mg, 32.1 μmol, 0.1 eq). The mixture was stirred at 25° C. for 1 hour. LC-MS showed tert-butyl 4-(4-chloro-1H-indazol-3-yl)-3-(trifluoromethyl) piperazine-1-carboxylate was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-TLC (Silica gel, petroleum ether/ethyl acetate=5/1) to give desired tert-butyl 4-[4-chloro-1-(4-isopropylsulfonylphenyl) sulfonyl-indazol-3-yl]-3-(trifluoromethyl) piperazine-1-carboxylate (75 mg, 115 μmol, 35.9% yield) as a yellow solid. MS (ESI): mass calcd. For C₂₄H₂₆ClF₃N₄O₄S 558.1, mass found 559.1 [M+H]⁺.

Step 5: To a solution of tert-butyl 4-[4-chloro-1-(4-isopropylsulfonylphenyl) sulfonyl-indazol-3-yl]-3-(trifluoromethyl) piperazine-1-carboxylate (75 mg, 115 μmol, 1 eq) in HCl/EtOAc (4 M, 4 mL, 139 eq). The mixture was stirred at 25° C. for 0.25 hour. LC-MS showed tert-butyl 4-[4-chloro-1-(4-isopropylsulfonylphenyl) sulfonyl-indazol-3-yl]-3-(trifluoromethyl) piperazine-1-carboxylate was consumed completely and desired mass was detected. The reaction mixture was filtered and the filter liquor was concentrated under reduced pressure to give desired 4-chloro-1-(4-isopropylsulfonylphenyl) sulfonyl-3-[2-(trifluoromethyl) piperazin-1-yl]indazole (76 mg, crude) as a white solid. MS (ESI): mass calcd. For C₁₉H₁₈ClF₃N₄O₂S 458.1, mass found 459.1 [M+H]⁺.

### Intermediate-L46: tert-butyl 4-chloro-3-iodo-1H-indazole-1-carboxylate

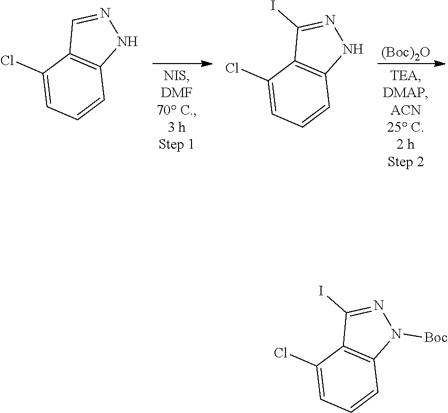 [SMILES: "(Boc)2O" ; Clc1cccc2[nH]ncc12 ; Clc1cccc2[nH]nc(I)c12 ; CC(C)(C)OC(=O)n1nc(I)c2c(Cl)cccc21]

Step 1: To a solution of 4-chloro-1H-indazole (5 g, 32.8 mmol, 1 eq) in DMF (20 mL) was added NIS (8.11 g, 36.1 mmol, 1.1 eq). The mixture was stirred at 70° C. for 3 hours. LC-MS showed 4-chloro-1H-indazole was consumed completely and desired mass was detected. The crude was added H₂O (50 mL), and extracted with MTBE (50 mL*3). The combined organic layers were washed with brine (20 mL), dried over Na₂SO₄, filtered and concentrated under reduced pressure to give desired 4-chloro-3-iodo-1H-indazole (8 g, crude) as a yellow solid. MS (ESI): mass calcd. For C₇H₄ClIN₂ 277.91 m/z found 278.8 [M+H]⁺.

Step 2: To a solution of 4-chloro-3-iodo-1H-indazole (8 g, 28.7 mmol, 1 eq) in ACN (80 mL) was added tertbutoxycarbonyl tert-butyl carbonate (9.40 g, 43.1 mmol, 9.90 mL, 1.5 eq) and TEA (5.81 g, 57.5 mmol, 8.00 mL, 2 eq) and DMAP (3.51 g, 28.7 mmol, 1 eq). The mixture was stirred at 25° C. for 2 hours. LC-MS showed 4-chloro-3-iodo-1H-indazole was consumed completely and desired mass was detected. The reaction mixture was concentrated to give the crude product. The residue was purified by flash silica gel chromatography (ISCO®; 40 g SepaFlash® Silica Flash Column, Eluent of 0˜20% EtOAc/petroleum ether gradient @80 mL/min) to give desired tert-butyl 4-chloro-3-iodo-indazole-1-carboxylate (9.5 g, 25.1 mmol, 87.4% yield) as a yellow solid. MS (ESI): mass calcd. For C₁₂H₁₂ClIN₂O₂ 377.96 m/z found 322.8 [M+H−56]⁺.

### Intermediate-L47: 3-(5-azaspiro [2.5]octan-5-yl)-4-chloro-1H-indazole

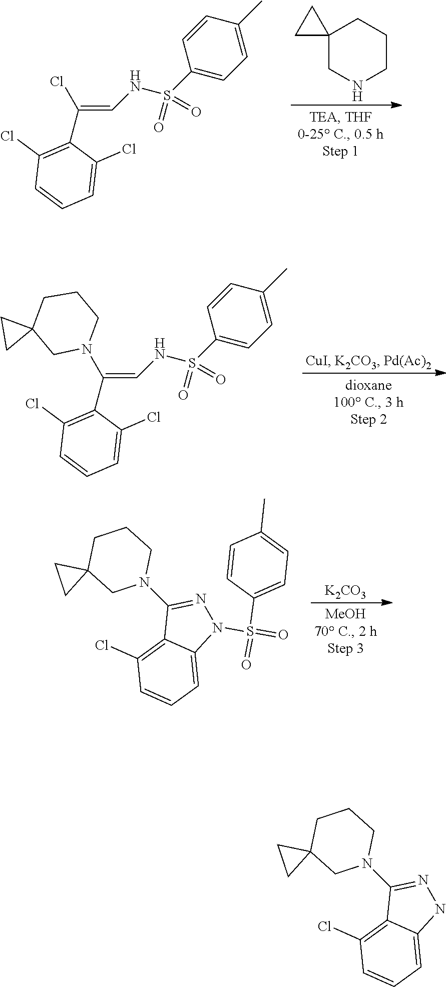

Step 1: To a solution of 5-azaspiro [2.5]octane (500 mg, 3.39 mmol, 1 eq, HCl) in THF (5 mL) was added dropwise TEA (514 mg, 5.08 mmol, 707 μL, 1.5 eq) at 25° C. After addition, the mixture was stirred at this temperature for 10 mins, and then (1Z)-2, 6-dichloro-N-(p-tolylsulfonyl) benzohydrazonoyl chloride (1.28 g, 3.39 mmol, 1 eq) in THF (20 mL) was added dropwise at 0° C. The resulting mixture was stirred at 25° C. for 20 mins. LC-MS showed (1Z)-2, 6-dichloro-N-(p-tolylsulfonyl) benzohydrazonoyl chloride was consumed completely and one main peak with desired mass was detected. Then it was separated between water (50 mL) and EtOAc (100 mL). The organic phase was separated, washed with brine (30 mL), dried over Na₂SO₄, filtered and concentrated under reduced pressure to give desired N—[(Z)-[5-azaspiro [2.5]octan-5-yl-(2, 6-dichlorophenyl) methylene]amino]-4-methyl-benzenesulfonamide (1.6 g, crude) as a yellow oil. MS (ESI): mass calcd. For C₂₁H₂₃C₁₂N₃O₂S 451.09, m/z found 452.1 [M+H]⁺.

Step 2: A mixture of N—[(Z)-[5-azaspiro [2.5]octan-5-yl-(2, 6-dichlorophenyl) methylene]amino]-4-methyl-benzenesulfonamide (1.6 g, 3.54 mmol, 1 eq), CuI (67.4 mg, 354 μmol, 0.1 eq), Pd(OAc)₂ (159 mg, 707 μmol, 0.2 eq) and K₂CO₃ (1.22 g, 8.84 mmol, 2.5 eq) in dioxane (20 mL) was degassed and purged with N₂ for 3 times, and then the mixture was stirred at 100° C. for 3 hours under N₂ atmosphere. LC-MS showed N—[(Z)-[5-azaspiro [2.5]octan-5-yl-(2, 6-dichlorophenyl) methylene]amino]-4-methyl-benzenesulfonamide was consumed completely and desired compound was detected. Then it was separated between water (50 mL) and EtOAc (100 mL). The organic phase was separated, washed with brine (50 mL), dried over Na₂SO₄, filtered and concentrated under reduced pressure to give desired 3-(5-azaspiro[2.5]octan-5-yl)-4-chloro-1-(p-tolylsulfonyl) indazole (1.4 g, crude) as a yellow oil. MS (ESI): mass calcd. For C₂₁H₂₂ClN₃O₂S 415.11, m/z found 416.2 [M+H]⁺.

Step 3: To a solution of 3-(5-azaspiro [2.5]octan-5-yl)-4-chloro-1-(p-tolylsulfonyl) indazole (1.4 g, 3.37 mmol, 1 eq) in MeOH (5 mL) was added K₂CO₃ (2.33 g, 16.8 mmol, 5 eq). The mixture was stirred at 70° C. for 2 hours. LC-MS showed 3-(5-azaspiro [2.5]octan-5-yl)-4-chloro-1-(p-tolylsulfonyl) indazole remained and desired compound was detected. The reaction mixture was added to water (20 mL), extracted with EtOAc (30 mL). The combined organic layers were washed with brine (20 mL) and dried over Na₂SO₄, filtered and concentrated under reduced pressure to give a residue. The residue was purified by prep-TLC (SiO₂, petroleum ether/EtOAc=3/1) to give desired 3-(5-azaspiro [2.5]octan-5-yl)-4-chloro-1H-indazole (330 mg, crude) as a yellow oil. MS (ESI): mass calcd. For C₁₄H₁₆ClN₃ 261.10, m/z found 262.1 [M+H]⁺.

### Intermediate-L48: N—[(Z)—[[7-(5-chloro-3-fluoro-2-pyridyl)-4, 7-diazaspiro [2.5]octan-4-yl]-(2, 4-dichloro-3-pyridyl) methylene]amino]-4-methyl-benzenesulfonamide

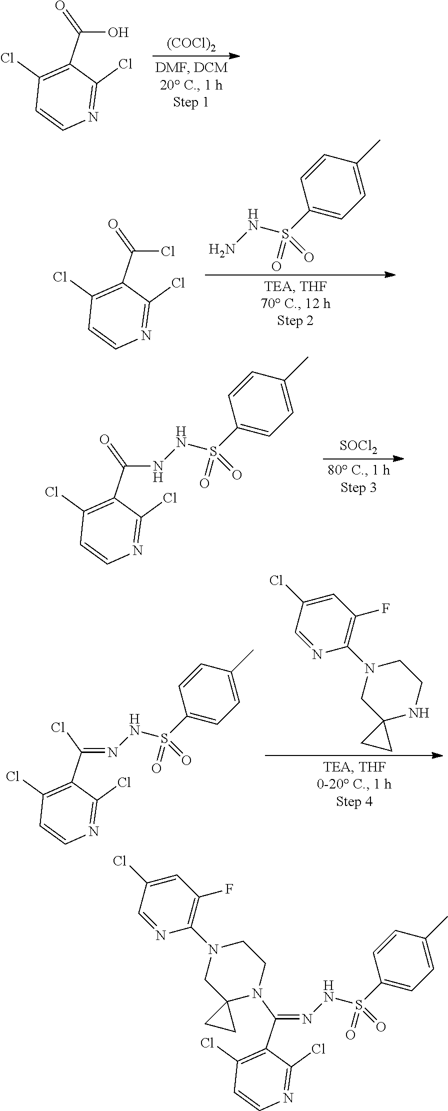

Step 1: To a solution of 2, 4-dichloropyridine-3-carboxylic acid (5 g, 26.0 mmol, 1 eq) in DCM (50 mL) was added oxalyl dichloride (13.2 g, 104 mmol, 9.12 mL, 4 eq) and DMF (95.2 mg, 1.30 mmol, 100 μL, 0.05 eq). The mixture was stirred at 20° C. for 1 hour. TLC (petroleum ether/EtOAc=2/1) indicated 2, 4-dichloropyridine-3-carboxylic acid was consumed completely and one new spot formed. The reaction was clean according to TLC. The reaction mixture was concentrated under reduced pressure to remove DCM to give desired 2, 4-dichloropyridine-3-carbonyl chloride (5 g, 23.8 mmol, 91.2% yield) as a yellow oil.

Step 2: To a solution of 2, 4-dichloropyridine-3-carbonyl chloride (5 g, 23.8 mmol, 1 eq) and 4-methylbenzenesulfonohydrazide (4.87 g, 26.1 mmol, 1.1 eq) in THF (50 mL) was added dropwise TEA (2.40 g, 23.8 mmol, 3.31 mL, 1 eq). The mixture was stirred at 70° C. for 12 hours. LC-MS showed desired mass was detected. The reaction mixture was diluted with H₂O (100 mL) and extracted with EtOAc (300 mL). The combined organic layers were washed with brine (100 mL), dried over anhydrous Na₂SO₄, filtered and concentrated under reduced pressure to give a residue. The residue was purified by flash silica gel chromatography (ISCO®; 80 g SepaFlash® Silica Flash Column, Eluent of 0˜50% EtOAc/petroleum ether gradient @80 mL/min) to give desired 2, 4-dichloro-N′-(p-tolylsulfonyl) pyridine-3-carbohydrazide (3 g, 8.33 mmol, 35.05% yield) as a yellow oil which was confirmed by LC-MS. MS (ESI): mass calcd. For C₁₃H₁₁C₁₂N₃O₃S 359.0, m/z found 359.9 [M+H]⁺.

Step 3: To a solution of 2, 4-dichloro-N′-(p-tolylsulfonyl) pyridine-3-carbohydrazide (500 mg, 1.39 mmol, 1 eq) in SOCl₂ (5 mL). The mixture was stirred at 80° C. for 1 hour. LC-MS showed 2, 4-dichloro-N′-(p-tolylsulfonyl) pyridine-3-carbohydrazide was consumed completely and one main peak with desired mass was detected. The reaction mixture was concentrated under reduced pressure to remove SOCl₂ to give desired (3Z)-2, 4-dichloro-N-(p-tolylsulfonyl) pyridine-3-carbohydrazonoyl chloride (500 mg, crude) as a yellow oil. MS (ESI): mass calcd. For C₁₃H₁₀C₁₃N₃O₂S 377.0, m/z found 427.0 [M+49+H]⁺.

Step 4: To a solution of 7-(5-chloro-3-fluoro-2-pyridyl)-4, 7-diazaspiro [2.5]octane (250 mg, 899 μmol, 1 eq, HCl) in THF (2 mL) was added TEA (273 mg, 2.70 mmol, 375 μL, 3 eq) at 0° C. Then (3Z)-2, 4-dichloro-N-(p-tolylsulfonyl) pyridine-3-carbohydrazonoyl chloride (408 mg, 1.08 mmol, 1.2 eq) in THF (4 mL) was added to the above mixture dropwise at 0° C. The mixture was stirred at 20° C. for 12 hours. LC-MS showed 7-(5-chloro-3-fluoro-2-pyridyl)-4, 7-diazaspiro [2.5]octane was consumed completely and one main peak with desired mass was detected. The reaction mixture was diluted with H₂O (5 mL) and extracted with EtOAc (100 mL). The combined organic layers were washed with brine (60 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜30% EtOAc/petroleum ether gradient @60 mL/min) to give desired N—[(Z)—[[7-(5-chloro-3-fluoro-2-pyridyl)-4, 7-diazaspiro [2.5]octan-4-yl]-(2, 4-dichloro-3-pyridyl) methylene]amino]-4-methyl-benzenesulfonamide (200 mg, 343 μmol, 38.1% yield) as a white solid which was confirmed by LC-MS. MS (ESI): mass calcd. For C₂₄H₂₂C₁₃FN₆O₂S 582.1, m/z found 583.1 [M+H]⁺.

### Intermediate-L49: 4-[4-chloro-6-fluoro-1-(p-tolylsulfonyl) indazol-3-yl]-4-azaspiro [2.5]octan-7-one

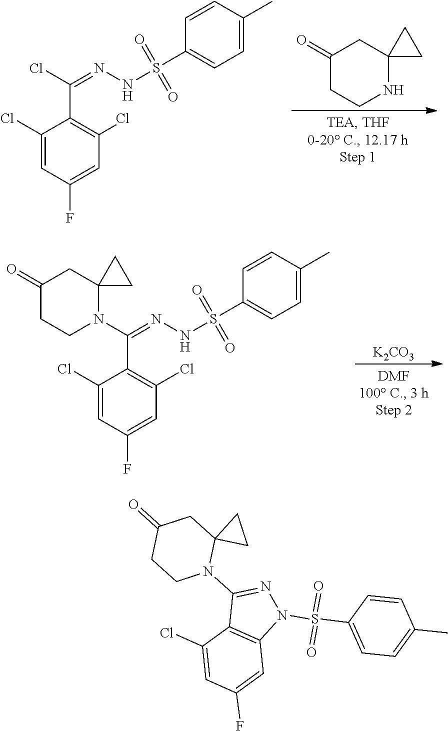

Step 1: To a solution of 4-azaspiro [2.5]octan-7-one (1.35 g, 8.34 mmol, 1.1 eq, HCl) in THF (10 mL) was added TEA (767 mg, 7.58 mmol, 1.06 mL, 1 eq). The mixture was stirred at 0° C. for 10 mins. Then (1E)-2,6-dichloro-4-fluoro-N-(p-tolylsulfonyl)benzohydrazonoyl chloride (3 g, 7.58 mmol, 1 eq) was added to the mixture. The mixture was stirred at 20° C. for 12 hours. LC-MS showed (1E)-2, 6-dichloro-4-fluoro-N-(p-tolylsulfonyl) benzohydrazonoyl chloride was consumed completely and one main peak with desired mass was detected. The reaction mixture was diluted with H₂O (30 mL) and extracted with EtOAc (150 mL). The combined organic layers were washed with brine (40 mL), dried over anhydrous Na₂SO₄, filtered and concentrated under reduced pressure to give desired N—[(E)-[(2, 6-dichloro-4-fluoro-phenyl)-(7-oxo-4-azaspiro [2.5]octan-4-yl)methylene]amino]-4-methyl-benzenesulfonamide (3.7 g, crude) as a yellow solid. MS (ESI): mass calcd. For C₂₁H₂₀C₁₂FN₃O₃S 483.1 m/z found 484.0 [M+H]⁺.

Step 2: To a solution of N—[(E)-[(2, 6-dichloro-4-fluoro-phenyl)-(7-oxo-4-azaspiro [2.5]octan-4-yl) methylene]amino]-4-methyl-benzenesulfonamide (3.7 g, 7.64 mmol, 1 eq) in DMF (30 mL) was added K₂CO₃ (10.6 g, 76.4 mmol, 10 eq). The mixture was stirred at 100° C. for 3 hours. LCMS showed N—[(E)-[(2, 6-dichloro-4-fluoro-phenyl)-(7-oxo-4-azaspiro [2.5]octan-4-yl) methylene]amino]-4-methyl-benzenesulfonamide was consumed completely and one main peak with desired mass was detected. The reaction mixture was diluted with H₂O (50 mL) and extracted with MTBE (300 mL). The combined organic layers were washed with brine (40 mL), dried over anhydrous Na₂SO₄, filtered and concentrated under reduced pressure to give a residue. The residue was purified by flash silica gel chromatography (ISCO®; 80 g SepaFlash® Silica Flash Column, Eluent of 0˜35% EtOAc/petroleum ether gradient @70 mL/min) to give desired 4-[4-chloro-6-fluoro-1-(p-tolylsulfonyl)indazol-3-yl]-4-azaspiro[2.5]octan-7-one (860 mg, 1.92 mmol, 25.1% yield) as a yellow solid which was confirmed by LC-MS. MS (ESI): mass calcd. For C₁₄H₁₃ClFN₃O 447.1 m/z found 448.1 [M+H]⁺.

### Intermediate-R1: 5-(tert-butylsulfonyl) thiophene-2-sulfonyl chloride

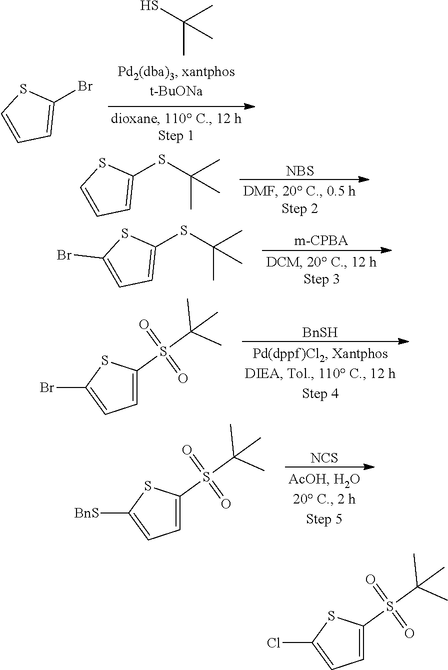

Step 1: A mixture of 2-methylpropane-2-thiol (332 mg, 3.68 mmol, 414 μL, 1.2 eq), 2-bromothiophene (500 mg, 3.07 mmol, 298 μL, 1 eq), DIEA (793 mg, 6.13 mmol, 1.07 mL, 2 eq), Xantphos (177 mg, 307 umol, 0.1 eq) and Pd(dppf)C₁₂ (56.1 mg, 76.7 μmol, 0.025 eq) in Tol. (10 mL) was degassed and purged with N₂ for 3 times, and then the mixture was stirred at 110° C. for 12 hours under the atmosphere nitrogen. TLC indicated 2-methylpropane-2-thiol was consumed completely and one new spot was formed. The reaction mixture was concentrated under reduced pressure to remove toluene. The reaction mixture was added to water (30 mL), extracted with EtOAc (30 mL*3). The combined organic layers were washed with brine (20 mL) and dried over Na₂SO₄, filtered and concentrated under reduced pressure to give a residue. The residue was purified by flash silica gel chromatography (ISCO®; 12 g SepaFlash® Silica Flash Column, Eluent of 0˜10% ethyl acetate/petroleum ether gradient @40 mL/min) to give desired 2-tert-butylsulfanylthiophene (250 mg, crude) as a yellow oil.

Step 2: To the solution of 2-tert-butylsulfanylthiophene (150 mg, 871 μmol, 1 eq) in DMF (5 mL) was added NBS (155 mg, 871 μmol, 1.0 eq) at 20° C. and the solution was stirred at 20° C. for 0.5 hour. TLC showed 2-tert-butylsulfanylthiophene was consumed completely and a new spot with lower polarity. The reaction was poured into water (10 mL) and extracted with MTBE (3*5 mL). The combined organics were concentrated to get a residue. The residue was purified by flash silica gel chromatography (ISCO®; 12 g SepaFlash® Silica Flash Column, Eluent of 0˜10% ethyl acetate/petroleum ether gradient @40 mL/min) to give desired 2-bromo-5-tert-butylsulfanyl-thiophene (178 mg, 709 μmol, 81.4% yield) as a yellow oil.

Step 3: To the solution of 2-bromo-5-tert-butylsulfanyl-thiophene (178 mg, 709 μmol, 1 eq) in DCM (10 mL) was added m-CPBA (1.44 g, 7.09 mmol, 85% purity, 10 eq) at 20° C. and the solution was stirred at 20° C. for 12 hours. TLC showed 2-bromo-5-tert-butylsulfanyl-thiophene was consumed completely and a new spot with larger polarity. The reaction was poured into water (10 mL) and extracted with MTBE (3*5 mL). The combined organics were washed with 1N NaOH (2*10 mL), dried over Na₂SO₄ and concentrated to get to give desired 2-bromo-5-tert-butylsulfonyl-thiophene (213 mg, crude) as a yellow solid.

Step 4: The mixture of 2-bromo-5-tert-butylsulfonyl-thiophene (213 mg, 752 μmol, 1 eq), BnSH (467 mg, 3.76 mmol, 441 μL, 5 eq), DIEA (194 mg, 1.50 mmol, 262 μL, 2 eq), Xantphos (87.0 mg, 150 μmol, 0.2 eq) and Pd(dppf)C₁₂ (55.0 mg, 75.2 μmol, 0.1 eq) in toluene (2 mL) was degassed with N₂ for 3 times and the mixture was stirred at 110° C. for 12 hours. TLC showed 2-bromo-5-tert-butylsulfonyl-thiophene was consumed completely and a main new spot was detected. The reaction was concentrated to get a residue. The residue was purified by flash silica gel chromatography (ISCO®; 40 g SepaFlash® Silica Flash Column, Eluent of 0-25% ethyl acetate/petroleum ether gradient @60 mL/min) to give desired 2-benzylsulfanyl-5-tert-butylsulfonyl-thiophene (210 mg, 643 μmol, 85.5% yield) as a red oil.

Step 5: To the solution of 2-benzylsulfanyl-5-tert-butylsulfonyl-thiophene (210 mg, 643 μmol, 1 eq) in AcOH (4 mL) and H₂O (0.5 mL) was added NCS (344 mg, 2.57 mmol, 4 eq) in portions at 20° C. and the solution was stirred at 20° C. for 2 hours. TLC showed 2-benzylsulfanyl-5-tert-butylsulfonyl-thiophene was consumed completely and a main new spot. The reaction was poured into water (10 mL) and extracted with MTBE (3*5 mL). The combined organics were washed with 1N NaOH (2*10 mL), dried over Na₂SO₄ and concentrated to get a residue. The residue was purified by flash silica gel chromatography (ISCO®; 40 g SepaFlash® Silica Flash Column, Eluent of 0-25% ethyl acetate/Petroleum ether gradient @60 mL/min) to give desired 5-tert-butylsulfonylthiophene-2-sulfonyl chloride (130 mg, 429 μmol, 66.75% yield) as a white solid.

### Intermediate-R2: 5-isopropylsulfonylthiophene-2-sulfonyl chloride

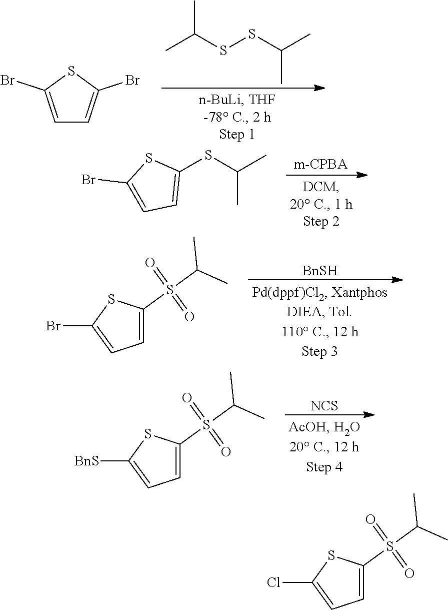

Step 1: To a solution of 2, 5-dibromothiophene (15 g, 62.0 mmol, 6.98 mL, 1 eq) 2-(isopropyldisulfanyl)propane (14.0 g, 93.0 mmol, 14.8 mL, 1.5 eq) in THF (300 mL) was added n-BuLi (2.5 M, 24.8 mL, 1 eq) under N₂. The mixture was stirred at −78° C. for 1 hour. TLC (petroleum ether/thyl acetate=1/0) indicated 2, 5-dibromothiophene was consumed completely and one new spot formed. The reaction mixture was quenched by addition sat. NH₄Cl aq. 800 mL, and extracted with EtOAc 800 mL (200 mL*4). The combined organic layers were washed with brine 400 mL, dried over Na₂SO₄, filtered and concentrated under reduced pressure to give desired 2-bromo-5-isopropylsulfanyl-thiophene (17 g, crude) as a yellow oil.

Step 2: To a solution of 2-bromo-5-isopropylsulfanyl-thiophene (17 g, 71.7 mmol, 1 eq) in DCM (300 mL) was added m-CPBA (50.9 g, 251 mmol, 85% purity, 3.5 eq) at 0° C. The mixture was stirred at 20° C. for 1 hour. TLC (petroleum ether/Ethyl acetate=3/1) indicated 2-bromo-5-isopropylsulfanyl-thiophene was consumed completely and one new spot formed. The reaction was clean according to TLC. Then it was partitioned between 300 mL of sat. Na₂SO₃ and 100 mL of DCM. The organic phase was separated, washed with 300 mL of sat. Na₂SO₃, 30 mL of brine, dried over Na₂SO₄, filtered and concentrated under reduced pressure to give the crude product. The residue was purified by column chromatography (SiO₂, petroleum ether/Ethyl acetate=1/0 to 0/1) to give desired 2-bromo-5-isopropylsulfonyl-thiophene (21 g, crude) as a yellow solid.

Step 3: A mixture of phenylmethanethiol (6.09 g, 49.0 mmol, 5.75 mL, 1.1 eq), 2-bromo-5-isopropylsulfonyl-thiophene (12 g, 44.6 mmol, 1 eq), DIEA (11.5 g, 89.2 mmol, 15.5 mL, 2 eq), Xantphos (2.58 g, 4.46 mmol, 0.1 eq) and Pd(dppf)C₁₂ (815 mg, 1.11 mmol, 0.025 eq) in Tol. (100 mL) was degassed and purged with N₂ for 3 times, and then the mixture was stirred at 110° C. for 12 hours under the atmosphere of nitrogen. TLC (petroleum ether/Ethyl acetate=3/1) indicated 2-bromo-5-isopropylsulfonyl-thiophene was consumed completely and one new spot formed. The reaction was clean according to TLC. The reaction mixture was concentrated under reduced pressure to remove Tol. The reaction mixture was added to water (500 mL), extracted with EtOAc (300 mL*3). The combined organic layers were washed with brine 300 mL and dried over Na₂SO₄, filtered and concentrated under reduced pressure to give a residue. The residue was purified by column chromatography (SiO₂, petroleum ether/Ethyl acetate=1/0 to 1/1) to give desired 2-benzylsulfanyl-5-isopropylsulfonyl-thiophene (10.8 g, crude) as an orange oil.

Step 4: To a solution of 2-benzylsulfanyl-5-isopropylsulfonyl-thiophene (10.8 g, 34.6 mmol, 1 eq) in AcOH (80 mL) and H₂O (20 mL) was added NCS (13.9 g, 104 mmol, 3 eq). The mixture was stirred at 20° C. for 12 hours. TLC (petroleum ether/Ethyl acetate=3/1) showed 2-benzylsulfanyl-5-isopropylsulfonyl-thiophene was consumed completely and one major new spot with larger polarity was detected. The reaction mixture was diluted with water 50 mL and extracted with EtOAc 450 mL (50 mL*3). The combined organic layers were washed with brine 50 mL, dried over anhydrous Na₂SO₄, filtered and concentrated under reduced pressure to give a residue. The residue was purified by column chromatography to give desired 5-isopropylsulfonylthiophene-2-sulfonyl chloride (7.8 g, crude) as a yellow oil.

### Intermediate-R3: 4-isopropylsulfonylbenzenesulfonyl chloride

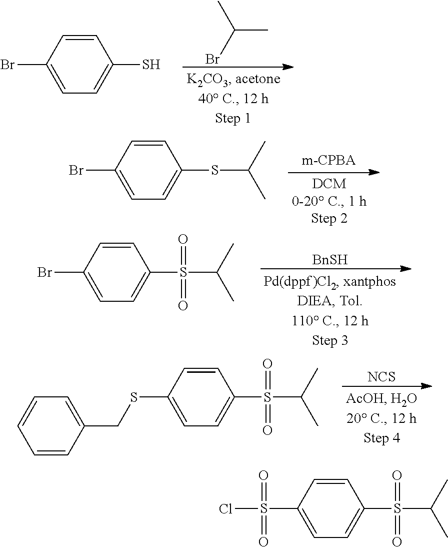

Step 1: A mixture of 4-bromobenzenethiol (10 g, 52.9 mmol, 1 eq), 2-bromopropane (13.0 g, 106 mmol, 9.93 mL, 2 eq), K₂CO₃ (25.6 g, 185 mmol, 3.5 eq) in acetone (100 mL) was degassed and purged with N₂ for 3 times, and then the mixture was stirred at 40° C. for 12 hours under the atmosphere of nitrogen. TLC (petroleum ether/Ethyl acetate=5/1) indicated 4-bromobenzenethiol was consumed completely and one new spot formed. The reaction was clean according to TLC. The reaction mixture was added to water (200 mL), extracted with EtOAc (100 mL*3). The combined organic layers were washed with brine 200 mL and dried over Na₂SO₄, filtered and concentrated under reduced pressure to give desired 1-bromo-4-isopropylsulfanyl-benzene (12 g, 51.9 mmol, 98.2% yield) as a brown oil.

Step 2: To a solution of 1-bromo-4-isopropylsulfanyl-benzene (1 g, 4.33 mmol, 1 eq) in DCM (10 mL) was added m-CPBA (2.63 g, 13.0 mmol, 85% purity, 3 eq) at 0° C. The mixture was stirred at 20° C. for 1 hour. TLC (petroleum ether/Ethyl acetate=4/1) indicated 1-bromo-4-isopropylsulfanyl-benzene was consumed completely and one new spot formed. The reaction was clean according to TLC. Then it was partitioned between 30 mL of sat. Na₂SO₃ and 100 mL of DCM. The organic phase was separated, washed with 30 mL of sat. Na₂SO₃, 30 mL of brine, dried over Na₂SO₄, filtered and concentrated under reduced pressure to give the crude product. The residue was purified by column chromatography (SiO₂, petroleum ether/Ethyl acetate=1/0 to 0/1) to give desired 1-bromo-4-isopropylsulfonyl-benzene (1 g, crude) as a white solid.

Step 3: The mixture of 1-bromo-4-isopropylsulfonyl-benzene (1 g, 3.80 mmol, 1 eq), BnSH (2.36 g, 19.0 mmol, 2.23 mL, 5 eq), DIEA (982 mg, 7.60 mmol, 1.32 mL, 2 eq), Xantphos (440 mg, 760 μmol, 0.2 eq) and Pd(dppf)C₁₂ (278 mg, 380 μmol, 0.1 eq) in toluene (20 mL) was degassed with N₂ for 3 times and the mixture was stirred at 110° C. for 12 hours. LC-MS showed 1-bromo-4-isopropylsulfonyl-benzene was consumed completely and a new spot. The reaction was concentrated to get a residue. The residue was purified by flash silica gel chromatography (ISCO®; 40 g SepaFlash® Silica Flash Column, Eluent of 0˜50% Ethylacetate/petroleum ether gradient @80 mL/min) to give desired benzylsulfanyl-4-isopropylsulfonyl-benzene (1.24 g, crude) as a yellow solid. MS (ESI): mass calcd. For C₁₆H₁₈O₂S₂ 306.07, m/z found 307.1 [M+H]⁺.

Step 4: To the solution of 1-benzylsulfanyl-4-isopropylsulfonyl-benzene (1.24 g, 4.05 mmol, 1 eq) in AcOH (24 mL) and H₂O (2.4 mL) was added NCS (2.16 g, 16.2 mmol, 4 eq) at 20° C. and the solution was stirred at 20° C. for 12 hours. TLC (petroleum ether/Ethyl acetate=2/1) showed 1-benzylsulfanyl-4-isopropylsulfonyl-benzene was consumed completely and a new spot. The reaction was quenched slowly with saturated NaHCO₃ solution (200 mL) and extracted with MTBE (2*30 mL). The combined organics were concentrated to get a residue. The residue was purified by flash silica gel chromatography (ISCO®; 40 g SepaFlash® Silica Flash Column, Eluent of 0˜50% ethyl acetate/petroleum ether gradient @80 mL/min) to give desired 4-isopropylsulfonylbenzenesulfonyl chloride (0.78 g, 2.76 mmol, 68.2% yield) as a white solid.

### Intermediate-R4: 4-(cyclopropylsulfonyl)benzene-1-sulfonyl chloride

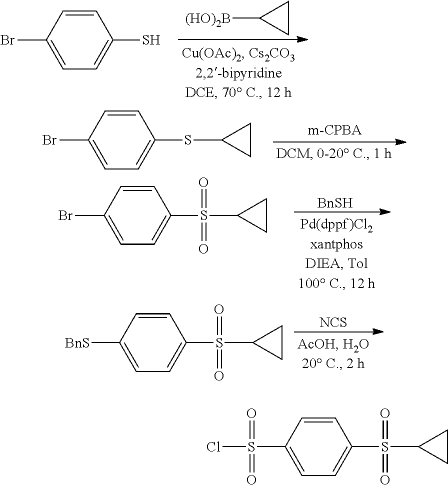

Step 1: The mixture of 4-bromobenzenethiol (0.5 g, 2.64 mmol, 1 eq), cyclopropylboronic acid (340 mg, 3.96 mmol, 636 μL, 1.5 eq), Cs₂CO₃ (860 mg, 2.64 mmol, 1 eq), 2,2′-bipyridine (412 mg, 2.64 mmol, 1 eq) and Cu(OAc)₂ (480 mg, 2.64 mmol, 1 eq) in DCE (10 mL) was stirred at 70° C. for 12 hours. TLC (petroleum ether/Ethyl acetate=5/1) indicated 4-bromobenzenethiol was consumed completely. The residue was added water (50 mL) and extracted with MTBE (2×50 mL). The combined organics were concentrated to get a residue. The residue was purified by flash silica gel chromatography (ISCO®; 12 g SepaFlash® Silica Flash Column, Eluent of 0˜10% ethyl acetate/petroleum ether gradient @60 mL/min) to give desired 1-bromo-4-cyclopropylsulfanyl-benzene (600 mg, crude) as a yellow oil. ¹H NMR (chloroform-d) δ: 7.40 (d, 2H), 7.23 (d, 2H), 2.11-2.22 (m, 1H), 1.05-1.14 (m, 2H), 0.64-0.74 (m, 2H)

Step 2: To the solution of 1-bromo-4-cyclopropylsulfanyl-benzene (600 mg, 2.62 mmol, 1 eq) in DCM (20 mL) was added m-CPBA (2.66 g, 13.1 mmol, 85% purity, 5 eq) at 0° C. by portions and stirred at 20° C. for 1 hour. The mixture was stirred at 20° C. for 1 hour. TLC showed 1-bromo-4-cyclopropylsulfanyl-benzene was consumed completely and desired spot. The reaction was filtered and the filtrate was washed with 1N NaOH (20 mL) and dry the organic layer with sodium sulfate. The mixture was filtered and the filtrate was concentrated to give desired 1-bromo-4-cyclopropylsulfonyl-benzene (600 mg, crude) as a yellow oil.

Step 3: A mixture of BnSH (2.66 g, 21.4 mmol, 2.51 mL, 9.32 eq), 1-bromo-4-cyclopropylsulfonyl-benzene (600 mg, 2.30 mmol, 1 eq), DIEA (594 mg, 4.60 mmol, 800 μL, 2 eq), Xantphos (133 mg, 230 μmol, 0.1 eq) and Pd(dppf)C₁₂ (42.0 mg, 57.4 μmol, 0.025 eq) in toluene (10 mL) was degassed and purged with N₂ for 3 times, and then the mixture was stirred at 110° C. for 12 hr under the atmosphere of atmosphere. TLC showed 1-bromo-4-cyclopropylsulfonyl-benzene was consumed completely. The reaction mixture was concentrated under reduced pressure to remove Tol. The reaction mixture was added to water (5 mL), extracted with EtOAc (10 mL*3). The combined organic layers were washed with brine (10 mL) and dried over Na₂SO₄, filtered and concentrated under reduced pressure to give a residue. The residue was purified by flash silica gel chromatography (ISCO®; 25 g SepaFlash® Silica Flash Column, Eluent of 0˜50% ethyl acetate/petroleum ether gradient @60 mL/min) to give desired 1-benzylsulfanyl-4-cyclopropylsulfonyl-benzene (600 mg, crude) as a yellow solid. ¹H NMR (chloroform-d) δ: 7.71-7.79 (m, 2H), 7.28-7.43 (m, 7H), 4.24 (s, 2H), 2.43 (m, 1H), 1.29-1.38 (m, 2H), 0.97-1.08 (m, 2H).

Step 4: To the solution of 1-benzylsulfanyl-4-cyclopropylsulfonyl-benzene (600 mg, 1.97 mmol, 1 eq) in AcOH (5 mL) and H₂O (0.5 mL) was added NCS (1.05 g, 7.88 mmol, 4 eq) at 20° C. and the solution was stirred at 20° C. for 2 hours. TLC showed 1-benzylsulfanyl-4-cyclopropylsulfonyl-benzene was consumed completely and a new spot. The reaction was quenched slowly with saturated NaHCO₃ solution (10 mL) and extracted with MTBE (2*10 mL). The combined organics were concentrated to get a residue. The combined organics were concentrated to get a residue. The residue was purified by flash silica gel chromatography (ISCO®; 12 g SepaFlash® Silica Flash Column, Eluent of 0˜50% ethyl acetate/petroleum ether gradient @60 mL/min) to give desired 4-cyclopropylsulfonylbenzenesulfonyl chloride (331 mg, crude) as a white solid.

### Intermediate-R5: 4-(1-fluoro-1-methyl-ethyl)sulfonylbenzenesulfonyl chloride

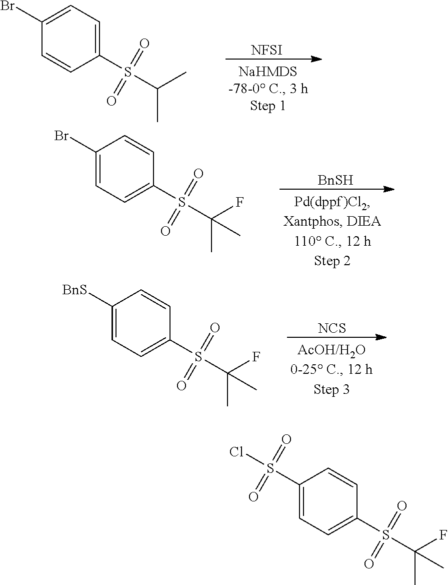

Step 1: To a solution of 1-bromo-4-isopropylsulfonyl-benzene (3 g, 11.4 mmol, 1 eq) in THF (30 mL) was added NaHMDS (1 M, 13.7 mL, 1.2 eq) at −78° C. under N₂. The mixture was stirred at 0° C. for 30 minutes. NFSI (5.46 g, 17.3 mmol, 1.52 eq) was added to the mixture at −78° C. The mixture was stirred at −78° C. for 30 minutes. TLC (petroleum ether/EtOAc=5/1) indicated 1-bromo-4-isopropylsulfonyl-benzene was consumed completely and one major new spot with lower polarity was detected. The reaction mixture was with H₂O (20 mL) and extracted with EtOAc (90 mL). The combined organic layers were washed with brine (20 mL), dried over anhydrous Na₂SO₄, filtered and concentrated under reduced pressure to give a residue. The residue was purified by flash silica gel chromatography (ISCO®; 80 g SepaFlash® Silica Flash Column, Eluent of 0˜10% EtOAc/petroleum ether gradient @60 mL/min) to give desired 1-bromo-4-(1-fluoro-1-methyl-ethyl)sulfonyl-benzene (750 mg, 2.67 mmol, 23.40% yield) as a yellow oil. MS (ESI): mass calcd. For C₉H₁₀BrFO₂S 279.96, m/z found 204.8 [M−74+H]⁺.

Step 2: A mixture of 1-bromo-4-(1-fluoro-1-methyl-ethyl)sulfonyl-benzene (750 mg, 2.67 mmol, 1 eq), phenylmethanethiol (364 mg, 2.93 mmol, 344 μL, 1.1 eq), DIEA (690 mg, 5.34 mmol, 929 μL, 2 eq), Pd(dppf)C₁₂ (48.8 mg, 66.7 μmol, 0.025 eq) and Xantphos (154 mg, 267 μmol, 0.1 eq) in Tol. (5 mL) was degassed and purged with N₂ for 3 times, and then the mixture was stirred at 110° C. for 12 hours under the atmosphere of nitrogen. TLC (petroleum ether/EtOAc=3/1) indicated 1-bromo-4-(1-fluoro-1-methyl-ethyl)sulfonyl-benzene was consumed completely and three new spots formed. The reaction mixture was concentrated under reduced pressure to remove Tol. The reaction mixture was added to water (10 mL), extracted with EtOAc (30 mL). The combined organic layers were washed with brine (10 mL) and dried over Na₂SO₄, filtered and concentrated under reduced pressure to give a residue. The residue was purified by prep-TLC (SiO₂, petroleum ether/EtOAc=3/1) to give desired 1-benzylsulfanyl-4-(1-fluoro-1-methyl-ethyl)sulfonyl-benzene (830 mg, 2.56 mmol, 95.9% yield) as a yellow oil.

Step 3: To a solution of 1-benzylsulfanyl-4-(1-fluoro-1-methyl-ethyl)sulfonyl-benzene (830 mg, 2.56 mmol, 1 eq) in AcOH (6 mL) and H₂O (1.5 mL) was added NCS (1.02 g, 7.67 mmol, 3 eq) at 0° C. The mixture was stirred at 20° C. for 12 hours. TLC (petroleum ether/EtOAc=3/1) indicated 1-benzylsulfanyl-4-(1-fluoro-1-methyl-ethyl)sulfonyl-benzene was consumed completely and three new spots formed. The reaction mixture was diluted with H₂O (5 mL) and extracted with EtOAc (10 mL). The combined organic layers were washed with brine (6 mL), dried over anhydrous Na₂SO₄, filtered and concentrated under reduced pressure to give a residue. The residue was purified by prep-TLC (SiO2, Petroleum ether/EtOAc=3/1) to give 4-(1-fluoro-1-methyl-ethyl)sulfonylbenzenesulfonyl chloride (440 mg, 1.46 mmol, 57.19% yield) as a white solid.

### Intermediate-R6: 4-(1, 1-difluoroethyl) benzenesulfonyl chloride

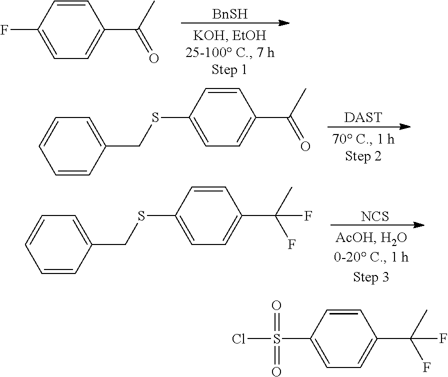

Step 1: KOH (3.25 g, 57.9 mmol, 1 eq) was added to a solution of phenylmethanethiol (7.91 g, 63.7 mmol, 7.46 mL, 1.1 eq) in EtOH (100 mL). The mixture was heated to reflux until the KOH had completely dissolved and then cooled to 25° C. A solution of 1-(4-fluorophenyl) ethanone (8 g, 57.9 mmol, 7.02 mL, 1 eq) in EtOH (20 mL) was then added dropwise and the mixture was heated to 100° C. for 7 hours. TLC (petroleum ether/EtOAc=5/1) indicated 1-(4-fluorophenyl) ethanone was consumed completely and one new spot formed. The crude was added H₂O (100 mL), and extracted with EtOAc (100 mL*3). The combined organic layers were washed with brine (100 mL), dried over Na₂SO₄, filtered and concentrated under reduced pressure to give a residue. The residue was purified by prep-TLC (Silica gel, petroleum ether/EtOAc=5/1) to give desired 1-(4-benzylsulfanylphenyl) ethanone (14 g, crude) as a brown oil.

Step 2: To a solution of 1-(4-benzylsulfanylphenyl) ethanone (12 g, 49.5 mmol, 1 eq) in DAST (120 mL). The mixture was stirred at 70° C. for 1 hour. TLC (petroleum ether/EtOAc=5/1) indicated 1-(4-benzylsulfanylphenyl) ethanone was consumed completely two new spots formed. Then it was partitioned between water (200 mL) and EtOAc (150 mL*3). The organic phase was separated, washed with brine (50 mL), dried over Na₂SO₄, filtered and concentrated under reduced pressure to give a residue. The residue was purified by flash silica gel chromatography (ISCO®; 12 SepaFlash® Silica Flash Column, Eluent of 0˜5% EtOAc/petroleum ether gradient @80 mL/min) to give desired 1-benzylsulfanyl-4-(1, 1-difluoroethyl) benzene (2.5 g, 9.46 mmol, 19.1% yield) as a white solid.

Step 3: To a solution of 1-benzylsulfanyl-4-(1, 1-difluoroethyl) benzene (2.5 g, 9.46 mmol, 1 eq) in AcOH (20 mL) and H₂O (5 mL) was added NCS (5.05 g, 37.8 mmol, 4 eq) at 0° C. The mixture was stirred at 20° C. for 1 hour. LC-MS showed 1-benzylsulfanyl-4-(1, 1-difluoroethyl) benzene was consumed completely and desired mass was detected (The sample was quenched with piperidine). The reaction mixture was filtered and the filter liquor was concentrated under reduced pressure to give a residue. The residue was purified by flash silica gel chromatography (ISCO®; 12 g SepaFlash® Silica Flash Column, Eluent of 0˜5% EtOAc/petroleum ether gradient @60 mL/min) to give desired 4-(1, 1-difluoroethyl) benzenesulfonyl chloride (1.33 g, 5.53 mmol, 58.4% yield) as a yellow oil. MS (ESI): mass calcd. For C₈H₇F₂SO₂Cl 239.98, mass found 290.1[M+49+H]⁺.

### Intermediate-R7: 4-(difluoromethylsulfonyl) benzenesulfonyl chloride

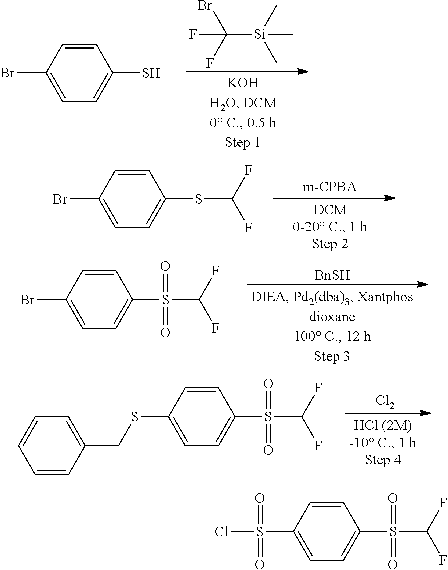

Step 1: To a solution of 4-bromobenzenethiol (2.0 g, 10.6 mmol, 1 eq) in DCM (10 mL) was added KOH (17.8 g, 63.5 mmol, 20% purity, 6 eq) with vigorous stirring. Then a solution of [bromo (difluoro) methyl]-trimethyl-silane (4.30 g, 21.2 mmol, 2 eq) in DCM (10 mL) was added into the mixture at 0° C. The mixture was stirred at 0° C. for 30 min. TLC (petroleum ether/Ethyl acetate=1/0) indicated 4-bromobenzenethiol remained and one new spot formed. The reaction was clean according to TLC. The reaction mixture was added to water (50 mL), extracted with EtOAc (30 mL*3). The combined organic layers were washed with brine 20 mL and dried over Na₂SO₄, filtered and concentrated under reduced pressure to give desired 1-bromo-4-(difluoromethylsulfanyl)benzene (2.7 g, crude) as a colourless oil.

Step 2: To a solution of 1-bromo-4-(difluoromethylsulfanyl) benzene (2.7 g, 11.3 mmol, 1 eq) in DCM (30 mL) was added m-CPBA (6.88 g, 33.9 mmol, 85% purity, 3 eq) at 0° C. The mixture was stirred at 20° C. for 1 hour. TLC (petroleum ether/Ethyl acetate=5/1) indicated 1-bromo-4-(difluoromethylsulfanyl) benzene was consumed completely and one new spot formed. The reaction was clean according to TLC. Then it was partitioned between 30 mL of sat. Na₂SO₃ and 100 mL of DCM. The organic phase was separated, washed with 30 mL of sat. NaHCO₃, 30 mL of brine, dried over Na₂SO₄, filtered and concentrated under reduced pressure to give the crude product. The residue was purified by column chromatography (SiO₂, petroleum ether/Ethyl acetate=1/0 to 10/1) to give desired 1-bromo-4-(difluoromethylsulfonyl) benzene (2 g, crude) as a yellow oil.

Step 3: A mixture of phenylmethanethiol (302 mg, 2.43 mmol, 285 μL, 1.1 eq), 1-bromo-4-(difluoromethylsulfonyl)benzene (600 mg, 2.21 mmol, 1 eq), DIEA (572 mg, 4.43 mmol, 771 μL, 2 eq), Xantphos (128 mg, 221 μmol, 0.1 eq) and Pd₂(dba)₃ (50.7 mg, 55.3 μmol, 0.025 eq) in dioxane (10 mL) was degassed and purged with N₂ for 3 times, and then the mixture was stirred at 100° C. for 12 hours under N₂ atmosphere. TLC (petroleum ether/Ethyl acetate=5/1) indicated 1-bromo-4-(difluoromethylsulfonyl) benzene was consumed completely and one new spot formed. The reaction was clean according to TLC. The reaction mixture was added to water (20 mL), extracted with EtOAc (30 mL*3). The combined organic layers were washed with brine 20 mL and dried over Na₂SO₄, filtered and concentrated under reduced pressure to give a residue. The residue was purified by prep-TLC (SiO₂, petroleum ether/Ethyl acetate=5/1) to give desired 1-benzylsulfanyl-4-(difluoromethylsulfonyl) benzene (300 mg, crude) as a yellow solid.

Step 4: To a solution of 1-benzylsulfanyl-4-(difluoromethylsulfonyl) benzene (50 mg, 159 μmol, 1 eq) in HCl (1 mL, 10% purity) was gradually introduced C₁₂ (5.00 g, 70.5 mmol, 443 eq) (15 psi) at −10° C. for 0.5 hour, and a white solid gradually precipitated; After the chlorine gas is stopped, the excess chlorine gas is purged with nitrogen at −10° C. for 0.5 hour. LC-MS showed 1-benzylsulfanyl-4-(difluoromethylsulfonyl) benzene was consumed completely and desired mass was detected. The crude was added NaHSO₃ (10 mL), and extracted with EtOAc 30 mL (10 mL*3). The combined organic layers were washed with brine 10 mL, dried over Na₂SO₄, filtered and concentrated under reduced pressure to give desired 4-(difluoromethylsulfonyl)benzenesulfonyl chloride (100 mg, crude) as a yellow solid. MS (ESI): mass calcd. For C₇H₅ClF₂O₄S 289.93, m/z found 300.0 [M+H+9]⁺.

### Intermediate-R8: 1-isopropylsulfonylpyrrole-3-sulfonyl chloride

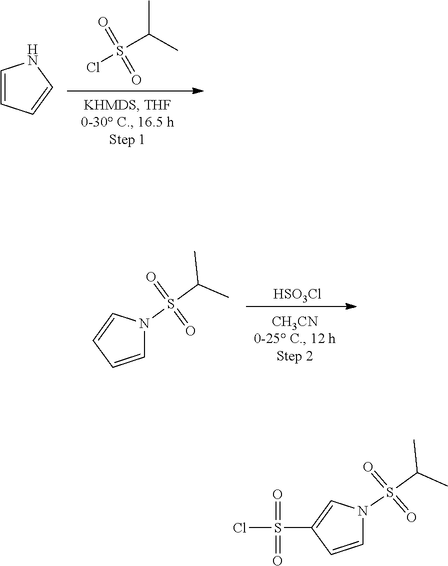

Step 1: To a solution of pyrrole (500 mg, 7.45 mmol, 517 μL, 1 eq) in THF (20 mL) was added hexamethyldisiliconyl potassium amino (KHMDS) (1 M, 7.45 mL, 1 eq) slowly at 0° C. under N₂. The reaction mixture was stirred at 0° C. for 30 mins under N₂. Then to the reaction mixture was added propane-2-sulfonyl chloride (1.06 g, 7.45 mmol, 830 μL, 1 eq) slowly at 0° C. under N₂. The reaction mixture was warmed to 30° C. and stirred at 30° C. for 16 hours under N₂. LC-MS showed pyrrole was consumed completely and desired mass was detected. The reaction mixture was partitioned between 100 mL of H₂O and 100 mL of EtOAc. The organic phase was separated, washed with 50 mL of brine, dried over Na₂SO₄, filtered and concentrated under reduced pressure to give desired 1-isopropylsulfonylpyrrole (900 mg, crude) as a yellow oil. MS (ESI): mass calcd. For C₇H₁₁NO₂S 173.05, m/z found 174.0 [M+H]⁺.

Step 2: A solution of 1-isopropylsulfonylpyrrole (100 mg, 577 μmol, 1 eq) in acetonitrile (2 mL) was cooled to 0° C. followed by the dropwise addition of sulfurochloridic acid (135 mg, 1.15 mmol, 76.9 μL, 2 eq). The resulting solution was allowed to warm to 25° C. for 12 hours. TLC (petroleum ether/Ethyl acetate=3/1) indicated 1-isopropylsulfonylpyrrole was consumed completely two new spots formed. The reaction mixture was quenched by addition H₂O 10 mL at 0° C., and then diluted with H₂O 5 mL and extracted with solvent 20 mL. The combined organic layers were washed with brine 50 mL, dried over Na₂SO₄, filtered and concentrated under reduced pressure to give a residue. The residue was purified by prep-TLC (SiO₂, petroleum ether/Ethyl acetate=3/1) to give desired 1-isopropylsulfonylpyrrole-3-sulfonyl chloride (57 mg, 210 μmol, 36.3% yield) as a yellow oil. ¹H NMR (400 MHz, CHLOROFORM-d) δ 7.85-7.80 (m, 1H), 7.22-7.17 (m, 1H), 6.84-6.80 (m, 1H), 3.56-3.50 (m, 1H), 1.29-1.24 (m, 6H).

### Intermediate-R9: 3-(1, 1-difluoroethyl) bicycle [1.1.1]pentane-1-sulfonyl fluoride

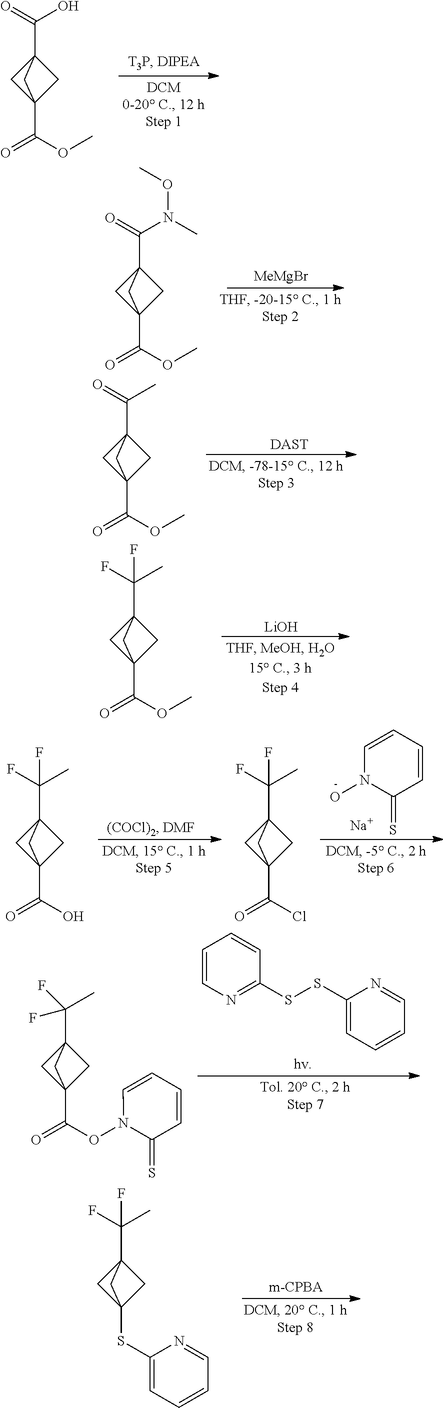

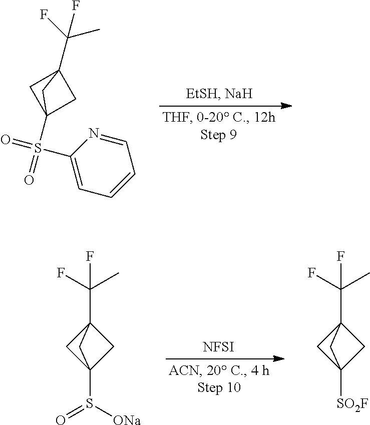

Step 1: To the solution of 3-methoxycarbonylbicyclo[1.1.1]pentane-1-carboxylic acid (2 g, 11.8 mmol, 1 eq), N-methoxymethanamine (1.15 g, 11.8 mmol, 1 eq, HCl) and DIPEA (9.11 g, 70.5 mmol, 12.3 mL, 6 eq) in DCM (50 mL) was added T₃P (12.7 g, 20.0 mmol, 11.9 mL, 50% purity, 1.70 eq) at 0° C. under the atmosphere of nitrogen. The solution was stirred at 20° C. for 12 hours. TLC showed 3-methoxycarbonylbicyclo [1.1.1]pentane-1-carboxylic acid was consumed completely and a new spot formed. The reaction was added DCM (50 mL) and washed with water (50 mL), 1N HCl (50 mL) and brine (50 mL). The organic was dried over anhydrous sodium sulfate and concentrated to get a residue. The residue was purified by flash silica gel chromatography (ISCO®; 40 g SepaFlash® Silica Flash Column, Eluent of 0˜50% ethyl acetate/petroleum ether gradient @60 mL/min) to give desired methyl 3-[methoxy(methyl)carbamoyl]bicyclo[1.1.1]pentane-1-carboxylate (2.3 g, 10.8 mmol, 91.8% yield) as a white solid. ¹H NMR (Chloroform-d) δ 3.69 (s, 3H), 3.67 (s, 3H), 3.18 (s, 3H), 2.38 (s, 6H)

Step 2: To a solution of methyl 3-[methoxy (methyl) carbamoyl]bicycle [1.1.1]pentane-1-carboxylate (1.8 g, 8.44 mmol, 1 eq) in THF (18 mL) was added MeMgBr (3 M, 2.81 mL, 1 eq) at −20° C. under the atmosphere of nitrogen. The mixture was stirred at 15° C. for 1 hour. TLC indicated methyl 3-[methoxy (methyl) carbamoyl]bicycle [1.1.1]pentane-1-carboxylate was consumed completely and one new spot formed. The reaction mixture was quenched by addition saturated aqueous NH₄Cl (50 mL) at 0° C. slowly under N₂ and stirred at 25° C. for 15 mins. THF was removed under vacuum. The resulting solution was diluted with water (30 mL), extracted with EtOAc (30 mL*3). The combined organic phase was washed with brine (30 mL*2), dried over Na₂SO₄, filtered and concentrated in vacuum. The residue was purified by flash silica gel chromatography (ISCO®; 12 g SepaFlash® Silica Flash Column, Eluent of 0˜50% ethyl acetate/petroleum ethergradient @80 mL/min) to give desired methyl 3-acetylbicyclo[1.1.1]pentane-1-carboxylate (700 mg, 4.16 mmol, 49.30% yield) as a light yellow solid.

Step 3: To a solution of methyl 3-acetylbicyclo [1.1.1]pentane-1-carboxylate (700 mg, 4.16 mmol, 1 eq) in DCM (10 mL) was added DAST (6.71 g, 41.6 mmol, 5.50 mL, 10 eq) at −78° C. The mixture was stirred at 15° C. for 12 hours. TLC indicated methyl 3-acetylbicyclo [1.1.1]pentane-1-carboxylate was consumed completely and one new spot formed. The reaction was quenched by sodium bicarbonate in water (50 ml), then the mixture was extracted with EtOAc (50 mL*3). The organic phase was separated, washed with brine (30 mL*2), dried over Na₂SO₄, filtered and concentrated under reduced pressure to give a residue. The residue was purified by flash silica gel chromatography (ISCO®; 12 g SepaFlash® Silica Flash Column, Eluent of 0˜50% ethyl acetate/petroleum ethergradient @80 mL/min) to give desired methyl 3-(1, 1-difluoroethyl) bicycle [1.1.1]pentane-1-carboxylate (450 mg, 2.37 mmol, 56.9% yield) as alight yellow solid. ¹H NMR (400 MHz, Chloroform-d) δ 3.71-3.68 (m, 3H), 2.14-2.09 (m, 6H), 1.63-1.47 (m, 3H).

Step 4: To a solution of methyl 3-(1, 1-difluoroethyl) bicycle [1.1.1]pentane-1-carboxylate (450 mg, 2.37 mmol, 1 eq) in H₂O (1 mL), MeOH (1 mL) and THF (3 mL) was added LiOH (113 mg, 4.73 mmol, 2 eq). The mixture was stirred at 15° C. for 3 hours. TLC indicated methyl 3-(1, 1-difluoroethyl) bicycle [1.1.1]pentane-1-carboxylate was consumed completely and one new spot formed. The pH value of the reaction mixture was adjust to 3˜4 with 1N HCl, then the reaction mixture was partitioned between H₂O (30 mL) and EtOAc (20 mL*3). The organic phase was separated, washed with brine (15 mL*2), dried over Na₂SO₄, filtered and concentrated under reduced pressure to give desired 3-(1,1-difluoroethyl) bicycle [1.1.1]pentane-1-carboxylic acid (300 mg, crude) as a white solid.

Step 5: To the solution of 3-(1, 1-difluoroethyl) bicycle [1.1.1]pentane-1-carboxylic acid (280 mg, 1.59 mmol, 1 eq) and DMF (11.6 mg, 159 μmol, 12.2 μL, 0.1 eq) in DCM (3 mL) was added (COCl)₂ (242 mg, 1.91 mmol, 167 μL, 1.2 eq) at 15° C. and the solution was stirred at 15° C. for 1 hour. TLC indicated 3-(1, 1-difluoroethyl) bicycle [1.1.1]pentane-1-carboxylic acid was consumed completely and a new spot was detected. The reaction was concentrated to give desired 3-(1,1-difluoroethyl)bicyclo[1.1.1]pentane-1-carbonyl chloride (300 mg, crude) as a yellow oil.

Step 6: To the solution of 3-(1, 1-difluoroethyl) bicycle [1.1.1]pentane-1-carbonyl chloride (300 mg, 1.54 mmol, 1 eq) in DCM (2 mL) was added sodium; 1-oxidopyridin-1-ium-2-thiolate (252 mg, 1.70 mmol, 207 μL, 1.1 eq) at −5° C. and the solution was stirred at −5° C. for 2 hours. TLC indicated 3-(1, 1-difluoroethyl) bicycle [1.1.1]pentane-1-carbonyl chloride was consumed completely. The reaction was concentrated to give desired (2-thioxo-1-pyridyl) 3-(1, 1-difluoroethyl) bicycle [1.1.1]pentane-1-carboxylate (430 mg, crude) as a yellow solid.

Step 7: The solution of (2-thioxo-1-pyridyl) 3-(1, 1-difluoroethyl) bicycle [1.1.1]pentane-1-carboxylate (430 mg, 1.51 mmol, 1 eq) and 2-(2-pyridyldisulfanyl) pyridine (996 mg, 4.52 mmol, 3 eq) in toluene (20 mL) was degassed with Ar for 3 times and the solution was irradiated with a 2000 W halogen lamp under argon atmosphere at 20° C. for 2 hours. LCMS showed (2-thioxo-1-pyridyl) 3-(1, 1-difluoroethyl) bicycle [1.1.1]pentane-1-carboxylate was consumed completely and desired mass was detected. The reaction was concentrated to get a residue. The residue was purified by flash silica gel chromatography (ISCO®; 40 g SepaFlash® Silica Flash Column, Eluent of 0˜50% ethyl acetate/petroleum ether gradient @80 mL/min) to give desired 2-[[3-(1, 1-difluoroethyl)-1-bicyclo [1.1.1]pentanyl]sulfanyl]pyridine (162 mg, crude) as a colorless oil. MS (ESI): mass calcd. For C₁₂H₁₃F₂SN 241.07 m/z found 242.0 [M+H]⁺.

Step 8: To the solution of 2-[[3-(1, 1-difluoroethyl)-1-bicyclo [1.1.1]pentanyl]sulfanyl]pyridine (160 mg, 663 μmol, 1 eq) in DCM (10 mL) was added m-CPBA (404 mg, 1.99 mmol, 85% purity, 3 eq) at 20° C. and the solution was stirred at 20° C. for 1 hour. LCMS showed desired mass was detected and 2-[[3-(1, 1-difluoroethyl)-1-bicyclo [1.1.1]pentanyl]sulfanyl]pyridine was consumed completely. The reaction was concentrated to get a residue. The residue was purified by flash silica gel chromatography (ISCO®; 40 g SepaFlash® Silica Flash Column, Eluent of 0˜50% ethyl acetate/petroleum ether gradient @80 mL/min) to give desired 2-[[3-(1, 1-difluoroethyl)-1-bicyclo [1.1.1]pentanyl]sulfanyl]pyridine (162 mg, crude) as a colorless oil. MS (ESI): mass calcd. For C₁₂H₁₃F₂SNO₂ 273.06 m/z found 274.0 [M+H]⁺.

Step 9: To the solution of NaH (22.0 mg, 549 μmol, 60% purity, 1.5 eq) in THF (2 mL) was added EtSH (0.3 g, 4.83 mmol, 357 μL, 13.2 eq) at 0° C. and the solution was stirred at 0° C. for 1 hour. To the solution was added 2-[[3-(1, 1-difluoroethyl)-1-bicyclo [1.1.1]pentanyl]sulfonyl]pyridine (100 mg, 366 μmol, 1 eq) and the solution was stirred at 20° C. for 11 hours. TLC showed 2-[[3-(1, 1-difluoroethyl)-1-bicyclo [1.1.1]pentanyl]sulfonyl]pyridine was consumed completely and a new spot was detected. The mixture was diluted with MTBE (10 mL), the precipitate was filtered, washed with MTBE (10 mL), and dried in vacuo to give desired [3-(1, 1-difluoroethyl)-1-bicyclo [1.1.1]pentanyl]sulfinyloxysodium (40 mg, crude) as a white solid.

Step 10: To the solution of [3-(1, 1-difluoroethyl)-1-bicyclo [1.1.1]pentanyl]sulfinyloxysodium (40 mg, 183 μmol, 1 eq) in ACN (2 mL) was added NFSI (86.7 mg, 275 μmol, 1.5 eq) at 20° C. and the solution was stirred at 20° C. for 4 hours. TLC showed [3-(1, 1-difluoroethyl)-1-bicyclo [1.1.1]pentanyl]sulfinyloxysodium was consumed completely and a new spot was detected. The reaction was concentrated to get a residue. The residue was purified by flash silica gel chromatography (ISCO®; 12 g SepaFlash® Silica Flash Column, Eluent of 0˜50% ethyl acetate/petroleum ether gradient @40 mL/min) to give desired 3-(1, 1-difluoroethyl) bicycle [1.1.1]pentane-1-sulfonyl fluoride (27.9 mg, 130 μmol, 71.1% yield) as a white solid. ¹H NMR (Chloroform-d) δ: 2.47 (s, 6H), 1.62 (m, 3H).

### Intermediate-R10: 3-(trifluoromethyl) bicycle [1.1.1]pentane-1-sulfonyl fluoride

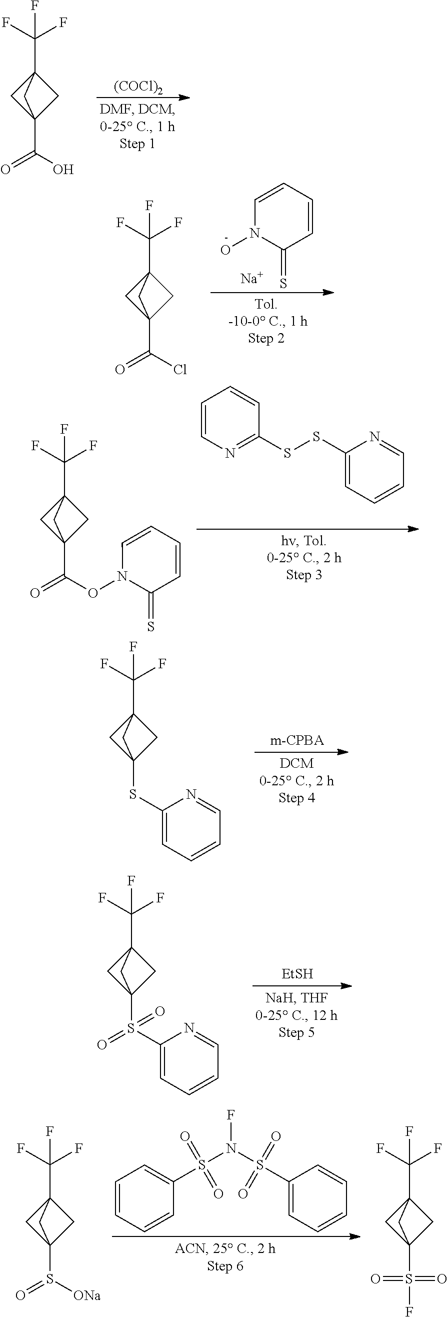

Step 1: The mixture of 3-(trifluoromethyl) bicycle [1.1.1]pentane-1-carboxylic acid (2 g, 11.1 mmol, 1 eq) in DCM (30 mL) was added DMF (81.2 mg, 1.11 mmol, 85.4 μL, 0.1 eq) was degassed and purged with N₂ for 3 times, and then was added (COCl)₂ (1.83 g, 14.4 mmol, 1.26 mL, 1.3 eq) at 0° C., and then the mixture was stirred at 25° C. for 1 hour under N₂ atmosphere. TLC (petroleum ether/Ethyl acetate=3/1) indicated 3-(trifluoromethyl) bicycle [1.1.1]pentane-1-carboxylic acid was consumed completely and one new spot formed (quenched with MeOH 0.5 mL). The reaction mixture was concentrated to give desired 3-(trifluoromethyl) bicycle [1.1.1]pentane-1-carbonyl chloride (2 g, crude) as a yellow oil.

Step 2: The mixture of sodium; 1-oxidopyridin-1-ium-2-thiolate (2.55 g, 17.1 mmol, 2.09 mL, 1.7 eq) in Tol. (5 mL) was degassed and purged with Ar for 3 times, and then was added 3-(trifluoromethyl) bicycle [1.1.1]pentane-1-carbonyl chloride (2 g, 10.1 mmol, 1 eq) at −10° C., and then the mixture was stirred at 0° C. for 1 hour under Ar atmosphere keep in dark place. LCMS showed 3-(trifluoromethyl) bicycle [1.1.1]pentane-1-carbonyl chloride was consumed completely and desired mass was detected. The reaction mixture was concentrated to give desired (2-thioxo-1-pyridyl) 3-(trifluoromethyl) bicycle [1.1.1]pentane-1-carboxylate (2 g, crude) as a yellow oil. MS (ESI): mass calcd. For C₁₂H₁₀F₃NO₂S 289.04 m/z found 290.2 [M+H]⁺.

Step 3: The mixture of (2-thioxo-1-pyridyl) 3-(trifluoromethyl) bicycle [1.1.1]pentane-1-carboxylate (2 g, 6.91 mmol, 1 eq) in Tol. (50 mL) was degassed and purged with Ar for 3 times, and then was added 2-(2-pyridyldisulfanyl)pyridine (3.81 g, 17.3 mmol, 2.5 eq) at 0° C., and then the mixture was stirred at 25° C. for 2 hours under argon atmosphere under 1000 w lamp. LCMS showed (2-thioxo-1-pyridyl) 3-(trifluoromethyl) bicycle [1.1.1]pentane-1-carboxylate was consumed completely and desired mass was detected. The reaction was concentrated to get a residue. The residue was added water (30 mL) and extracted with EtOAc (3*50 mL). The residue was purified by column chromatography (SiO₂, petroleum ether/Ethyl acetate=97/3 to 90:10) to give desired 2-[[3-(trifluoromethyl)-1-bicyclo [1.1.1]pentanyl]sulfanyl]pyridine (1.4 g, 5.71 mmol, 82.6% yield) as a yellow solid. MS (ESI): mass calcd. For C₁₁H₁₀F₃NS 245.05 m/z found 246.2 [M+H]⁺.

Step 4: The mixture of 2-[[3-(trifluoromethyl)-1-bicyclo [1.1.1]pentanyl]sulfanyl]pyridine (1.3 g, 5.30 mmol, 1 eq) in DCM (50 mL) was added m-CPBA (3.23 g, 15.9 mmol, 85% purity, 3 eq) at 0° C. The mixture was stirred at 25° C. for 2 hours. LCMS showed 2-[[3-(trifluoromethyl)-1-bicyclo [1.1.1]pentanyl]sulfanyl]pyridine was consumed completely and desired mass was detected. The reaction mixture was quenched by addition sat. aq. Na₂SO₃ (30 mL) at 0° C., and extracted with DCM (3*50 mL), then the combined organic layers was washed with sat. aq. Na₂CO₃ (20 mL). The residue was purified by column chromatography (SiO₂, petroleum ether/Ethyl acetate=10/1 to 5:1). The reaction mixture was concentrated to give desired 2-[[3-(trifluoromethyl)-1-bicyclo [1.1.1]pentanyl]sulfonyl]pyridine (1.1 g, 3.97 mmol, 74.9% yield) as a white solid. MS (ESI): mass calcd. For C₁₁H₁₀F₃NO₂S 277.04 m/z found 278.2 [M+H]⁺.

Step 5: The mixture of NaH (238 mg, 5.95 mmol, 60% purity, 1.5 eq) in THF (15 mL) was degassed and purged with Ar for 3 times, EtSH (986 mg, 15.9 mmol, 1.17 mL, 4 eq) was added dropwise, and the mixture was stirred at 0° C. for 1 hour, and then the mixture was added 2-[[3-(trifluoromethyl)-1-bicyclo[1.1.1]pentanyl]sulfonyl]pyridine (1.1 g, 3.97 mmol, 1 eq) at 0° C., and then the mixture was stirred at 25° C. for 11 hours under Ar atmosphere. TLC (petroleum ether/Ethyl acetate=3/1) indicated 2-[[3-(trifluoromethyl)-1-bicyclo [1.1.1]pentanyl]sulfonyl]pyridine was consumed completely and one new spot formed. The reaction mixture was concentrated under reduced pressure to give a residue. The reaction mixture was washed with MTBE (10 mL), and the filter cake was concentrated under reduced pressure to give a residue. The reaction mixture was concentrated to give desired [3-(trifluoromethyl)-1-bicyclo [1.1.1]pentanyl]sulfinyloxysodium (500 mg, 2.25 mmol, 56.7% yield) as a white solid.

Step 6: The mixture of [3-(trifluoromethyl)-1-bicyclo [1.1.1]pentanyl]sulfinyloxysodium (500 mg, 2.25 mmol, 1 eq) in ACN (20 mL) was added N-(benzenesulfonyl)-N-fluoro-benzenesulfonamide (1.06 g, 3.38 mmol, 1.5 eq). The mixture was stirred at 25° C. for 2 hours. TLC (petroleum ether/Ethyl acetate=1/1) indicated [3-(trifluoromethyl)-1-bicyclo [1.1.1]pentanyl]sulfinyloxysodium was consumed completely and one new spot formed. The residue was added water (30 mL) and extracted with EtOAc (3*50 mL). The residue was purified by column chromatography (SiO₂, petroleum ether/Ethyl acetate=20/1 to 10:1) to give desired 3-(trifluoromethyl) bicycle [1.1.1]pentane-1-sulfonyl fluoride (130 mg, 596 μmol, 26.5% yield) as a white solid.

### Intermediate-R11: 3-fluorobicyclo [1.1.1]pentane-1-sulfonyl fluoride

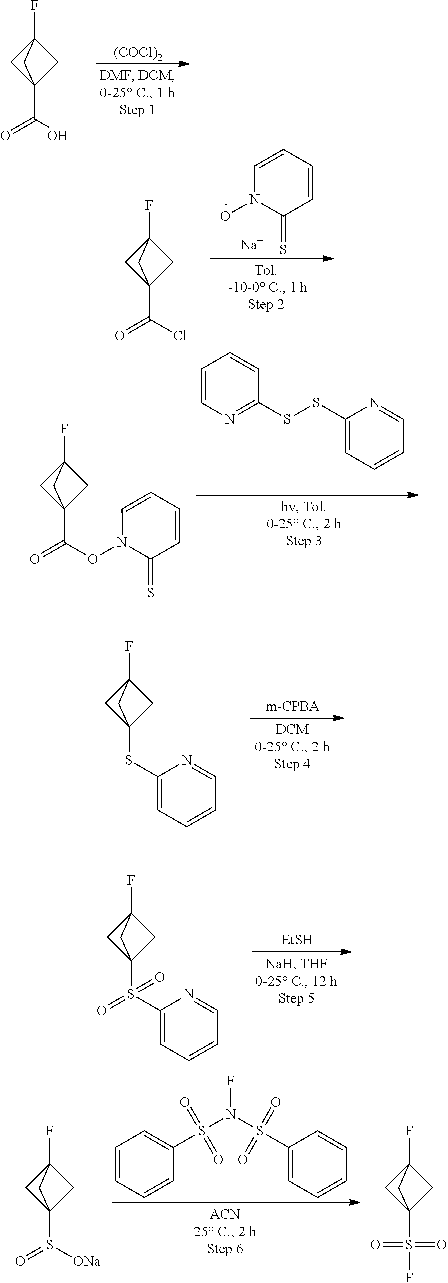

Step 1: The mixture of 3-fluorobicyclo [1.1.1]pentane-1-carboxylic acid (3.6 g, 27.7 mmol, 1 eq) in DCM (100 mL) was added DMF (202 mg, 2.77 mmol, 213 μL, 0.1 eq) was degassed and purged with N₂ for 3 times, and then was added (COCl)₂ (5.27 g, 41.5 mmol, 3.63 mL, 1.5 eq) at 0° C., and then the mixture was stirred at 25° C. for 1 hour under N₂ atmosphere. TLC (petroleum ether/Ethyl acetate=3/1) indicated 3-fluorobicyclo [1.1.1]pentane-1-carboxylic acid was consumed completely and one new spot formed (quenched with MeOH 0.5 mL). The reaction mixture was concentrated to give desired 3-fluorobicyclo [1.1.1]pentane-1-carbonyl chloride (3.6 g, crude) as a yellow oil.

Step 2: The mixture of sodium; 1-oxidopyridin-1-ium-2-thiolate (4.34 g, 29.1 mmol, 3.55 mL, 1.2 eq) in Tol. (50 mL) was degassed and purged with Ar for 3 times, and then was added 3-fluorobicyclo [1.1.1]pentane-1-carbonyl chloride (3.6 g, 24.2 mmol, 1 eq) at −10° C., and then the mixture was stirred at 0° C. for 1 hour under Ar atmosphere keep in dark place. LCMS showed sodium; 1-oxidopyridin-1-ium-2-thiolate was consumed completely and desired mass was detected. The reaction mixture was concentrated to give desired (2-thioxo-1-pyridyl) 3-fluorobicyclo [1.1.1]pentane-1-carboxylate (3.6 g, crude) as a yellow oil. MS (ESI): mass calcd. For C₁₁H₁₀FNO₂S 239.04 m/z found 240.2[M+H]⁺.

Step 3: The mixture of (2-thioxo-1-pyridyl) 3-fluorobicyclo [1.1.1]pentane-1-carboxylate (3.6 g, 15.1 mmol, 1 eq) in Tol. (100 mL) was degassed and purged with Ar for 3 times, and then was added 2-(2-pyridyldisulfanyl)pyridine (8.29 g, 37.6 mmol, 2.5 eq) at 0° C., and then the mixture was stirred at 25° C. for 2 hours under argon atmosphere under 1000 w lamp. LCMS showed (2-thioxo-1-pyridyl) 3-fluorobicyclo [1.1.1]pentane-1-carboxylate was consumed completely and desired mass was detected. The reaction was concentrated to get a residue. The residue was added water (30 mL) and extracted with EtOAc (3*50 mL). The residue was purified by column chromatography (SiO₂, petroleum ether/Ethyl acetate=93/7 to 90/10). The reaction mixture was concentrated to give desired 2-[(3-fluoro-1-bicyclo [1.1.1]pentanyl) sulfanyl]pyridine (600 mg, 3.07 mmol, 20.4% yield) as a yellow solid. MS (ESI): mass calcd. For C₁₀H₁₀FNS 195.05 m/z found 196.2 [M+H]⁺.

Step 4: The mixture of 2-[(3-fluoro-1-bicyclo [1.1.1]pentanyl) sulfanyl]pyridine (600 mg, 3.07 mmol, 1 eq) in DCM (20 mL) was added m-CPBA (1.87 g, 9.22 mmol, 85% purity, 3 eq) at 0° C. The mixture was stirred at 25° C. for 2 hours. LCMS showed 2-[(3-fluoro-1-bicyclo [1.1.1]pentanyl) sulfonyl]pyridine was consumed completely and desired mass was detected. The reaction mixture was quenched by addition sat. aq. Na₂SO₃ 30 mL at 0° C., and extracted with DCM (3*50 mL), then the combined organic layers was washed with sat. aq. Na₂CO₃ (20 mL). The residue was purified by column chromatography (SiO₂, petroleum ether/Ethyl acetate=10/1 to 5/1) to give desired 2-[(3-fluoro-1-bicyclo [1.1.1]pentanyl) sulfonyl]pyridine (500 mg, 2.20 mmol, 71.6% yield) as a white solid. MS (ESI): mass calcd. For C₁₀H₁₀FNO₂S 227.04 m/z found 228.2 [M+H]⁺.

Step 5: The mixture of NaH (132 mg, 3.30 mmol, 60% purity, 1.5 eq) in THF (20 mL) was degassed and purged with Ar for 3 times, EtSH (2.05 g, 33.0 mmol, 2.44 mL, 15 eq) was added dropwise, and the mixture was stirred at 0° C. for 1 hour, and then the mixture was added 2-[(3-fluoro-1-bicyclo [1.1.1]pentanyl) sulfonyl]pyridine (500 mg, 2.20 mmol, 1 eq) at 0° C., and then the mixture was stirred at 25° C. for 11 hours under Ar atmosphere. TLC (petroleum ether/Ethyl acetate=3/1) indicated 2-[(3-fluoro-1-bicyclo [1.1.1]pentanyl) sulfonyl]pyridine was consumed completely and one new spot formed. The reaction mixture concentrated under reduced pressure to give a residue. The reaction mixture was washed with MTBE (10 mL), and the filter cake concentrated under reduced pressure to give a residue. The reaction mixture was concentrated to give desired (3-fluoro-1-bicyclo [1.1.1]pentanyl) sulfinyloxysodium (340 mg, 1.97 mmol, 89.8% yield) as a white solid.

Step 6: The mixture of (3-fluoro-1-bicyclo [1.1.1]pentanyl) sulfinyloxysodium (340 mg, 1.97 mmol, 1 eq) in ACN (15 mL) was added N-(benzenesulfonyl)-N-fluoro-benzenesulfonamide (934 mg, 2.96 mmol, 1.5 eq). The mixture was stirred at 25° C. for 2 hours. TLC (petroleum ether/Ethyl acetate=1/1) indicated (3-fluoro-1-bicyclo [1.1.1]pentanyl) sulfinyloxysodium was consumed completely and one new spot formed. The residue was added water (30 mL) and extracted with EtOAc (3*50 mL). The residue was purified by column chromatography (SiO₂, petroleum ether/Ethyl acetate=20/1 to 10/1) to give desired 3-fluorobicyclo [1.1.1]pentane-1-sulfonyl fluoride (100 mg, 595 μmol, 30.1% yield) as a white solid.

### Intermediate-R12: 3-fluorobicyclo [1.1.1]pentane-1-sulfonyl fluoride

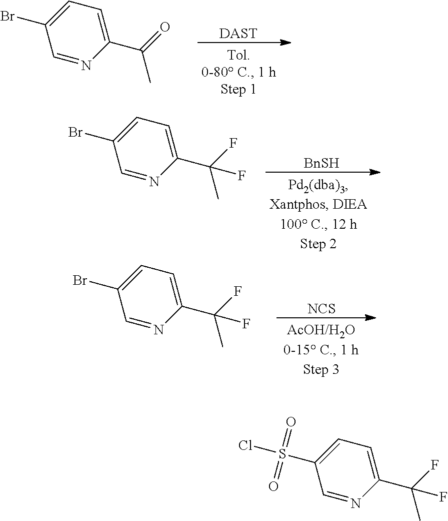

Step 1: To a solution of 1-(5-bromo-2-pyridyl) ethanone (1 g, 5.00 mmol, 1 eq) in Tol. (10 mL) was added DAST (4.03 g, 25.0 mmol, 3.30 mL, 5 eq) at 0° C. The mixture was stirred at 80° C. for 1 hour. TLC indicated 1-(5-bromo-2-pyridyl) ethanone was consumed completely one new spot formed. The reaction was quenched with ice-water (10 g) and extracted with EtOAc (2*10 mL). The combined organics were concentrated to get a residue. The residue was purified by prep-TLC (SiO₂, petroleum ether/EtOAc=5/1) to give desired 5-bromo-2-(1, 1-difluoroethyl) pyridine (360 mg, 1.62 mmol, 32.4% yield) as a yellow oil.

Step 2: A mixture of 5-bromo-2-(1, 1-difluoroethyl) pyridine (360 mg, 1.62 mmol, 1 eq), phenylmethanethiol (302 mg, 2.43 mmol, 286 μL, 1.5 eq), DIEA (419 mg, 3.24 mmol, 565 μL, 2 eq), Xantphos (93.8 mg, 162 μmol, 0.1 eq) and Pd₂(dba)₃ (149 mg, 162 μmol, 0.1 eq) in dioxane (5 mL) was degassed and purged with N₂ for 3 times, and then the mixture was stirred at 100° C. for 12 hours under N₂ atmosphere. LC-MS showed 5-bromo-2-(1, 1-difluoroethyl) pyridine was consumed completely and desired mass was detected. The reaction mixture was partitioned between water (10 mL) and EtOAc (20 mL). The organic phase was separated, washed with brine (20 mL), dried over Na₂SO₄, filtered and concentrated under reduced pressure to give a residue. The residue was purified by prep-TLC (SiO₂, petroleum ether/EtOAc=5/1) to give desired 5-benzylsulfanyl-2-(1, 1-difluoroethyl) pyridine (280 mg, 1.06 mmol, 65.1% yield) as a yellow oil. MS (ESI): mass calcd. For C₁₄H₁₃F₂NS 265.1, m/z found 266.1 [M+H]⁺.

Step 3: To a solution of 5-benzylsulfanyl-2-(1, 1-difluoroethyl) pyridine (130 mg, 490 μmol, 1 eq) in AcOH (0.8 mL) and H₂O (0.2 mL) was added NCS (262 mg, 1.96 mmol, 4 eq) at 0° C. The mixture was stirred at 15° C. for 1 hour. LC-MS showed 5-benzylsulfanyl-2-(1, 1-difluoroethyl) pyridine was consumed completely and desired mass was detected. TLC indicated 5-benzylsulfanyl-2-(1, 1-difluoroethyl) pyridine was consumed completely one new spot formed. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether/EtOAc=5/1) to give desired 6-(1, 1-difluoroethyl) pyridine-3-sulfonyl chloride (44 mg, 182 μmol, 37.2% yield) as a white solid. MS (ESI): mass calcd. For C₇H₆ClF₂NO₂S 241.0, m/z found 564.1 [M+H]⁺.

### Intermediate-R13: 2-isopropylsulfonylthiazole-4-sulfonyl chloride

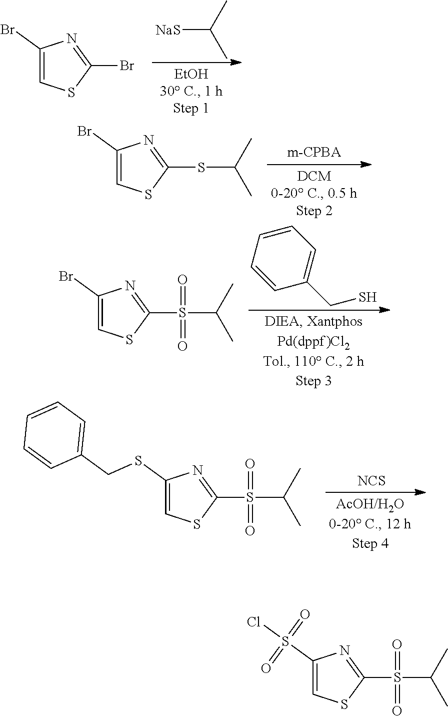

Step 1: To a solution of 2, 4-dibromothiazole (2.48 g, 10.2 mmol, 1 eq) in EtOH (30 mL) was added isopropylsulfanylsodium (1 g, 10.2 mmol, 2.95 mL, 1 eq). The mixture was stirred at 30° C. for 1 hour. LC-MS showed 2, 4-dibromothiazole was consumed completely and one main peak with desired mass was detected. The reaction mixture was concentrated under reduced pressure to remove EtOH. The residue was purified by prep-TLC (SiO₂, petroleum ether/EtOAc=10/1) to give desired 4-bromo-2-isopropylsulfanyl-thiazole (1.46 g, 6.13 mmol, 60.2% yield) as a white solid. MS (ESI): mass calcd. For C₆H₈BrNS₂ 236.93, m/z found 238.0 [M+H]⁺.

Step 2: To a solution of 4-bromo-2-isopropylsulfanyl-thiazole (1.46 g, 6.13 mmol, 1 eq) in DCM (25 mL) was added m-CPBA (7.47 g, 36.8 mmol, 85% purity, 6 eq) at 0° C. The mixture was stirred at 20° C. for 0.5 hour. LC-MS showed 4-bromo-2-isopropylsulfanyl-thiazole was consumed completely and one main peak with desired mass was detected. Then it was partitioned between 150 mL of sat. Na₂SO₃ and 300 mL of DCM. The organic phase was separated, washed with 150 mL of sat. NaHCO₃, 15 mL of brine, dried over Na₂SO₄, filtered and concentrated under reduced pressure to give desired 4-bromo-2-isopropylsulfonyl-thiazole (2.4 g, crude) as a white solid. MS (ESI): mass calcd. For C₆H₈BrNO₂S₂ 268.92, m/z found 270.0 [M+H]⁺.

Step 3: A mixture of 4-bromo-2-isopropylsulfonyl-thiazole (600 mg, 2.22 mmol, 1 eq), phenylmethanethiol (303 mg, 2.44 mmol, 286 μL, 1.1 eq), DIEA (574 mg, 4.44 mmol, 774 μL, 2 eq), Xantphos (129 mg, 222 μmol, 0.1 eq) and Pd(dppf)C₁₂ (40.6 mg, 55.5 μmol, 0.025 eq) in Tol. (5 mL) was degassed and purged with N₂ for 3 times, and then the mixture was stirred at 110° C. for 2 hours under N₂ atmosphere. LC-MS showed 4-bromo-2-isopropylsulfonyl-thiazole was consumed completely and one main peak with desired mass was detected. The reaction mixture was concentrated under reduced pressure to remove Tol. The mixture was cooled at 25° C. and added H₂O (100 mL), and extracted with EtOAc (100 mL). The combined organic layers were washed with brine (50 mL), dried over Na₂SO₄, filtered and concentrated under reduced pressure to give a residue. The residue was purified by prep-TLC (SiO₂, petroleum ether/EtOAc=2/1) to give desired 4-benzylsulfanyl-2-isopropylsulfonyl-thiazole (630 mg, 2.01 mmol, 90.5% yield) as a yellow oil. MS (ESI): mass calcd. For C₁₃H₁₅NO₂S₃ 313.03, m/z found 313.9 [M+H]⁺.

Step 4: To a solution of 4-benzylsulfanyl-2-isopropylsulfonyl-thiazole (300 mg, 957 μmol, 1 eq) in AcOH (8 mL) and H₂O (2 mL) was added NCS (383 mg, 2.87 mmol, 3 eq) at 0° C. The mixture was stirred at 20° C. for 12 hours. TLC (petroleum ether/EtOAc=2/1) indicated 4-benzylsulfanyl-2-isopropylsulfonyl-thiazole was consumed completely and one new spot formed. The reaction mixture was concentrated under reduced pressure to remove AcOH. The residue was diluted with water (15 mL) and extracted with EtOAc (30 mL). The combined organic layers were washed with brine (15 mL), dried over Na₂SO₄, filtered and concentrated under reduced pressure to give a residue. The residue was purified by prep-TLC (SiO₂, petroleum ether/EtOAc=2/1) to give desired 2-isopropylsulfonylthiazole-4-sulfonyl chloride (210 mg, 725 μmol, 75.7% yield) as a yellow oil.

### Intermediate-R14: 1-(dimethylsulfamoyl) pyrrole-3-sulfonyl chloride

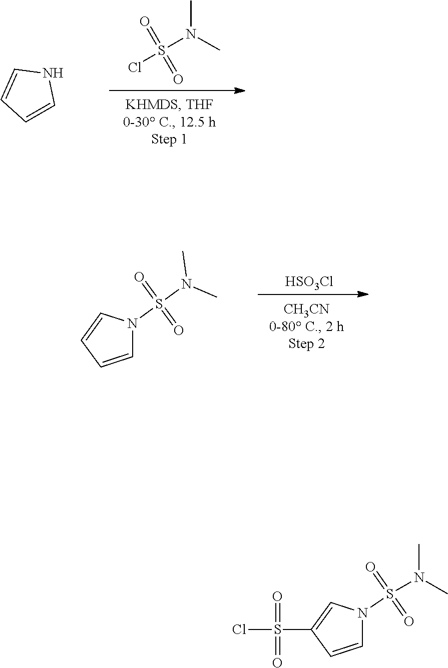

Step 1: To a solution of pyrrole (2 g, 29.8 mmol, 2.07 mL, 1 eq) in THF (20 mL) was added KHMDS (1 M, 29.8 mL, 1 eq) slowly at 0° C. under N₂. The reaction mixture was stirred at 0° C. for 30 mins under N₂. Then to the reaction mixture was added N, N-dimethylsulfamoyl chloride (4.28 g, 29.8 mmol, 3.19 mL, 1 eq) slowly at 0° C. under N₂. The reaction mixture was warmed to 30° C. and stirred at 30° C. for 12 hours under N₂. LC-MS showed pyrrole was consumed completely and desired mass was detected. The reaction mixture was partitioned between 200 mL of H₂O and 200 mL of EtOAc. The organic phase was separated, washed with 100 mL of brine, dried over Na₂SO₄, filtered and concentrated under reduced pressure to give desired N, N-dimethylpyrrole-1-sulfonamide (5.4 g, crude) as a yellow oil. MS (ESI): mass calcd. For C₆H₁₀N₂O₂S 174.05, m/z found 175.0 [M+H]⁺.

Step 2: To a solution of N, N-dimethylpyrrole-1-sulfonamide (2.5 g, 14.4 mmol, 1 eq) in ACN (40 mL) was added HSO₃Cl (3.34 g, 28.7 mmol, 1.91 mL, 2 eq) slowly at 0° C. The mixture was stirred at 80° C. for 2 hours. TLC (SiO₂, petroleum ether/EtOAc=3/1) indicated N, N-dimethylpyrrole-1-sulfonamide was consumed completely and one new spot formed. The reaction mixture was quenched by addition water (100 mL) at 0° C., and then diluted with water (100 mL) and extracted with EtOAc (100 mL). The combined organic layers were washed with brine (100 mL), dried over Na₂SO₄, filtered and concentrated under reduced pressure to give a residue. The residue was purified by column chromatography (SiO₂, petroleum ether/Ethyl acetate=1/0 to 1/1) to give desired 1-(dimethylsulfamoyl) pyrrole-3-sulfonyl chloride (160 mg, crude) as a yellow oil.

### Intermediate-R15: 2, 4, 5-trideuterio-1-isopropylsulfonyl-pyrrole-3-sulfonyl chloride

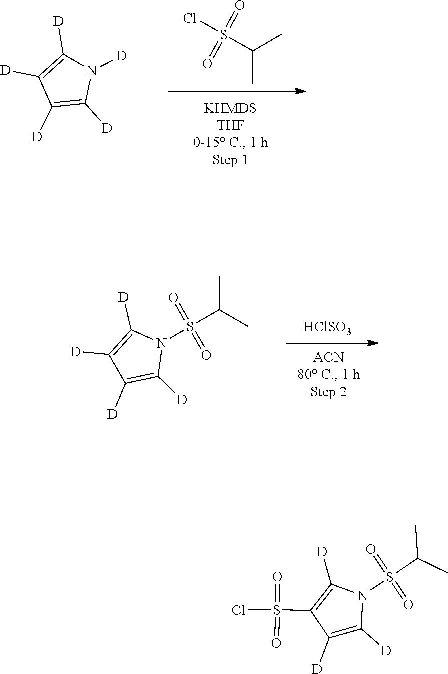

Step 1: To the solution of 1, 2, 3, 4, 5-pentadeuteriopyrrole (500 mg, 6.93 mmol, 1 eq) in THF (15 mL) was added KHMDS (1 M, 13.9 mL, 2 eq) at 0° C. under N₂ and the mixture was stirred at 0° C. for 0.5 hour. To the solution was added propane-2-sulfonyl chloride (1.48 g, 10.4 mmol, 1.16 mL, 1.5 eq) stirred at 0° C. and the solution was stirred at 15° C. for 0.5 hour. The residue was purified by flash silica gel chromatography (ISCO®; 40 g SepaFlash® Silica Flash Column, Eluent of 0˜25% Ethylacetate/petroleum ether gradient @40 mL/min) to give desired N-2,3,4,5-tetradeuterio-1-isopropylsulfonyl-pyrrole (500 mg, 2.82 mmol, 40.7% yield) as a colorless oil.

Step 2: To the solution of 2, 3, 4, 5-tetradeuterio-1-isopropylsulfonyl-pyrrole (500 mg, 2.82 mmol, 1 eq) in ACN (10 mL) was added HSO₃Cl (1.64 g, 14.1 mmol, 5 eq) and the solution was stirred at 80° C. for 1 hour. The reaction was poured into water (20 mL) and extracted with MTBE (2*10 mL). The combined organics were concentrated to get a residue. The residue was purified by flash silica gel chromatography (ISCO®; 20 g SepaFlash® Silica Flash Column, Eluent of 0˜20% Ethylacetate/petroleum ether gradient @50 mL/min) to give desired 2, 4, 5-trideuterio-1-isopropylsulfonyl-pyrrole-3-sulfonyl chloride (173 mg, 630 μmol, 22.32% yield) as a white solid.

### Intermediate-R16: 1-tert-butylsulfonylpyrrole-3-sulfonyl chloride

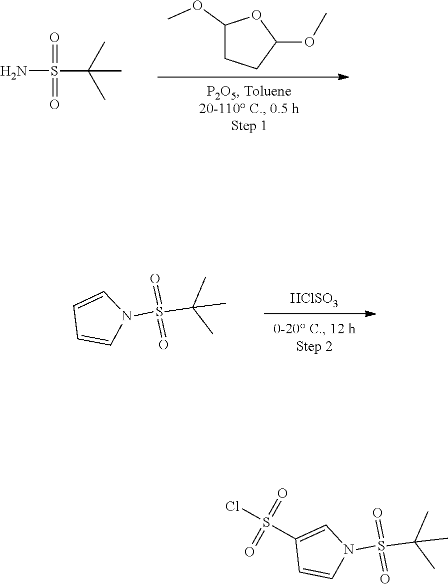

Step 1: To the solution of 2-methylpropane-2-sulfonamide (5 g, 36.4 mmol, 1 eq) and P₂O₅ (5.17 g, 36.4 mmol, 2.25 mL, 1 eq) in toluene (50 mL) was added 2,5-dimethoxytetrahydrofuran (7.22 g, 54.7 mmol, 7.08 mL, 1.5 eq) at 20° C. and the mixture was stirred at 110° C. for 0.5 hour. LCMS showed 2, 5-dimethoxytetrahydrofuran was consumed completely and desired mass was detected. The reaction was filtered and the filtrate was concentrated to get a residue. The residue was purified by flash silica gel chromatography (ISCO®; 40 g SepaFlash® Silica Flash Column, Eluent of 0˜25% EtOAc/petroleum ether gradient @40 mL/min) to give 1-tert-butylsulfonylpyrrole (4.2 g, crude) as a white solid. MS (ESI): mass calcd. For C₈H₁₃NO₂S 187.07 found 188.0 [M+H]⁺.

Step 2: To the solution of 1-tert-butylsulfonylpyrrole (4.2 g, 22.4 mmol, 1 eq) in ACN (100 mL) was added HSO₃Cl (13.1 g, 112 mmol, 7.47 mL, 5 eq) at 0° C. and the solution was stirred at 20° C. for 12 hours. TLC (SiO₂, petroleum ether/Ethyl acetate=2/1) showed 1-tert-butylsulfonylpyrrole was consumed completely and a new spot. The reaction was added into water (10 mL) and extracted with MTBE (3*10 mL). The organics were concentrated to get a residue. The residue was purified by flash silica gel chromatography (ISCO®; 40 g SepaFlash® Silica Flash Column, Eluent of 0˜50% Ethylacetate/petroleum ether gradient @80 mL/min) to give desired 1-tert-butylsulfonylpyrrole-3-sulfonyl chloride (580 mg, crude) as a white solid.

### Intermediate-R17: 1-cyclopropylsulfonylpyrrole-3-sulfonyl chloride

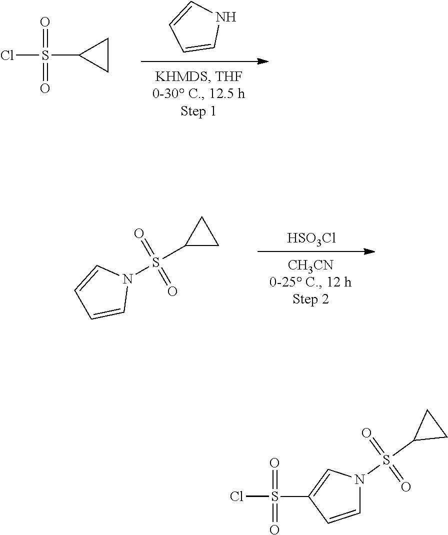 [SMILES: O=S(=O)(Cl)C1CC1 ; c1cc[nH]c1 ; O=S(=O)(C1CC1)n1cccc1 ; "HSO3Cl" ; O=S(=O)(Cl)c1ccn(S(=O)(=O)C2CC2)c1]

Step 1: To a solution of pyrrole (2 g, 29.8 mmol, 2.07 mL, 1 eq) in THF (20 mL) was added KHMDS (1 M, 29.8 mL, 1 eq) slowly at 0° C. under N₂. The reaction mixture was stirred at 0° C. for 30 mins under N₂. Then to the reaction mixture was added cyclopropanesulfonyl chloride (4.19 g, 29.8 mmol, 3.32 mL, 1 eq) slowly at 0° C. under N₂. The reaction mixture was warmed to 30° C. and stirred at 30° C. for 12 hours under N₂. LC-MS showed pyrrole was consumed completely and desired mass was detected. The reaction mixture was partitioned between H₂O (100 mL) and EtOAc (100 mL). The organic phase was separated, washed with brine (50 mL), dried over Na₂SO₄, filtered and concentrated under reduced pressure to give the crude product. The residue was purified by flash silica gel chromatography (ISCO®; 80 g SepaFlash® Silica Flash Column, Eluent of 0˜5% EtOAc/petroleum ether gradient @100 mL/min) to give desired 1-cyclopropylsulfonylpyrrole (2.2 g, 12.9 mmol, 43.1% yield) as a yellow oil. MS (ESI): mass calcd. For C₇H₉NO₂S 171.04, m/z found 172.1 [M+H]⁺.

Step 2: To a solution of 1-cyclopropylsulfonylpyrrole (2.2 g, 12.9 mmol, 1 eq) in ACN (25 mL) was added HSO₃Cl (2.99 g, 25.7 mmol, 1.71 mL, 2 eq) slowly at 0° C. The mixture was stirred at 25° C. for 12 hours. TLC (petroleum ether/EtOAc=3/1) indicated 1-cyclopropylsulfonylpyrrole was consumed completely and one new spot formed. The reaction mixture was quenched by addition water (30 mL) at 0° C., and then diluted with water (50 mL) and extracted with EtOAc (50 mL). The combined organic layers were washed with brine (50 mL), dried over Na₂SO₄, filtered and concentrated under reduced pressure to give desired 1-cyclopropylsulfonylpyrrole-3-sulfonyl chloride (680 mg, crude) as a black oil.

### Intermediate-R18: 1-isopropylsulfonylpyrazole-3-sulfonyl chloride

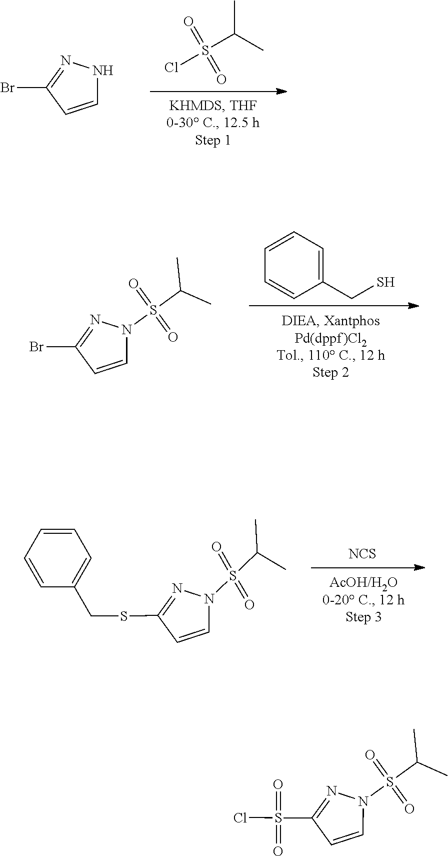

Step 1: To a solution of 3-bromo-1H-pyrazole (2 g, 13.6 mmol, 1 eq) in THF (10 mL) was added KHMDS (1 M, 13.6 mL, 1 eq) slowly at 0° C. under N₂. The mixture was stirred at 0° C. for 30 mins under N₂ atmosphere. Then the reaction mixture was added propane-2-sulfonyl chloride (1.94 g, 13.6 mmol, 1.52 mL, 1 eq) slowly at 0° C. under N₂. The mixture was warmed to 30° C. and stirred at 30° C. for 12 hours. LC-MS showed 3-bromo-1H-pyrazole was consumed completely and desired mass was detected. The reaction mixture was quenched by addition NH₄Cl solvent (40 mL) at 0° C., and then diluted with H₂O (20 mL) and extracted with EtOAc (40 mL). The combined organic layers were dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜1% DCM/MeOH @40 mL/min) to give desired 3-bromo-1-isopropylsulfonyl-pyrazole (2.95 g, 11.7 mmol, 85.7% yield) as a yellow oil. MS (ESI): mass calcd. For C₆H₉BrN₂O₂S 251.96 m/z found 252.9 [M+H]⁺.

Step 2: A mixture of 3-bromo-1-isopropylsulfonyl-pyrazole (2.95 g, 11.7 mmol, 1 eq), phenylmethanethiol (1.59 g, 12.8 mmol, 1.50 mL, 1.1 eq), DIEA (4.52 g, 35.0 mmol, 6.09 mL, 3 eq), Pd(dppf)C₁₂ (853 mg, 1.17 mmol, 0.1 eq) and Xantphos (1.35 g, 2.33 mmol, 0.2 eq) in Tol. (30 mL) was degassed and purged with N₂ for 3 times, and then the mixture was stirred at 110° C. for 12 hours under N₂ atmosphere. LC-MS showed 3-bromo-1-isopropylsulfonyl-pyrazole was consumed completely and desired mass was detected. The reaction mixture was partitioned between water (30 mL) and EtOAc (100 mL). The organic phase was separated, washed with brine (20 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜7% EtOAc/petroleum ether gradient @120 mL/min) to give desired 3-benzylsulfanyl-1-isopropylsulfonyl-pyrazole (3.0 g, 10.1 mmol, 86.8% yield) as a yellow oil. MS (ESI): mass calcd. For C₁₃H₁₆N₂O₂S₂ 296.07 m/z found 297.1 [M+H]⁺.

Step 3: To a solution of 3-benzylsulfanyl-1-isopropylsulfonyl-pyrazole (2.0 g, 6.75 mmol, 1 eq) in AcOH (20 mL) and H₂O (5 mL) was added NCS (2.70 g, 20.2 mmol, 3 eq) at 0° C. The mixture was stirred at 20° C. for 12 hours. TLC (petroleum ether/Ethyl acetate=2/1) indicated 4-benzylsulfanyl-2-isopropylsulfonyl-thiazole was consumed completely and one new spot formed. The reaction mixture was concentrated under reduced pressure to remove AcOH. The residue was diluted with H₂O (10 mL) and extracted with EtOAc (20 mL). The combined organic layers were washed with saline (20 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜23% EtOAc/petroleum ether gradient @120 mL/min) to give desired 1-isopropylsulfonylpyrazole-3-sulfonyl chloride (900 mg, 3.30 mmol, 48.9% yield) as a white solid.

### Intermediate-R19: 1H-1, 2, 4-triazole-3-sulfonyl chloride

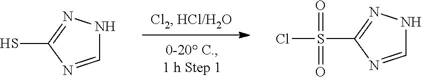 [SMILES: Sc1nc[nH]n1 ; O=S(=O)(Cl)c1nc[nH]n1]

Step 1: To the solution of 1H-1, 2, 4-triazole-3-thiol (5 g, 49.4 mmol, 1 eq) in HCl (2 M, 90 mL, 3.64 eq) was gradually introduced C₁₂ (60 g, 846 mmol, 17.1 eq) (15 psi) at 0° C. for 0.5 hour, and a white solid gradually precipitated. After the chlorine gas is stopped, the excess chlorine gas is purged with nitrogen at 0° C. for 0.5 hour. The reaction mixture was filtered and the filter cake was washed with water (50 mL) and dried under reduce pressure to give desired 1H-1, 2, 4-triazole-3-sulfonyl chloride (4.27 g, 25.5 mmol, 51.5% yield) as a white solid. ¹H NMR (400 MHz, DMSO-d₆) δ 9.08 (s, 1H).

### Intermediate-R20: 1-isopropylsulfonyl-2, 5-dimethyl-pyrrole-3-sulfonyl chloride

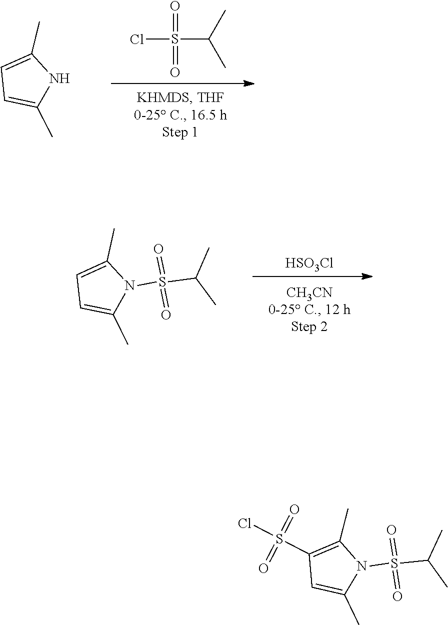

Step 1: To a solution of propane-2-sulfonyl chloride (1.50 g, 10.5 mmol, 1.17 mL, 1 eq) in THF (20 mL) was added KHMDS (1 M, 10.5 mL, 1 eq) slowly at 0° C. under N₂. The mixture was stirred at 0° C. for 30 mins under N₂. Then to the reaction mixture was added 2, 5-dimethyl-1H-pyrrole (1 g, 10.5 mmol, 1.07 mL, 1 eq) slowly at 0° C. under N₂. The reaction mixture was warmed to 25° C. and stirred at 25° C. for 16 hours under N₂. LC-MS showed propane-2-sulfonyl chloride was consumed completely and desired mass was detected. The reaction mixture was quenched by addition H₂O 40 mL, and extracted with EtOAc 80 mL (20 mL*4). The combined organic layers were washed with brine 20 mL, dried over Na₂SO₄, filtered and concentrated under reduced pressure to give a residue. The residue was purified by column chromatography (SiO₂, petroleum ether/Ethyl acetate=1/0 to 0/1) to give desired 1-isopropylsulfonyl-2, 5-dimethyl-pyrrole (600 mg, crude) as a yellow solid. MS (ESI): mass calcd. For C₉H₁₅NO₂S 201.08 m/z found 202.1 [M+H]⁺.

Step 2: A solution of 1-isopropylsulfonyl-2, 5-dimethyl-pyrrole (300 mg, 1.49 mmol, 1 eq) in CH₃CN (5 mL) was cooled to 0° C. followed by the dropwise addition of HSO₃Cl (347 mg, 2.98 mmol, 198 μL, 2 eq). The resulting solution was allowed to warm to 25° C. for 12 hours. TLC (SiO₂, petroleum ether/Ethyl acetate=3/1) indicated 1-isopropylsulfonyl-2, 5-dimethyl-pyrrole was consumed completely and one new spot formed. The reaction was clean according to TLC. The reaction mixture was filtered and the filtrate was concentrated under reduced pressure to give a residue. The reaction mixture was quenched by addition H₂O 40 mL, and extracted with EtOAc 40 mL (10 mL*4). The combined organic layers were washed with brine 20 mL, dried over Na₂SO₄, filtered and concentrated under reduced pressure to give a residue. The residue was purified by prep-TLC (SiO₂, petroleum ether/Ethyl acetate=3/1) to give desired 1-isopropylsulfonyl-2, 5-dimethyl-pyrrole-3-sulfonyl chloride (40 mg, crude) as a white solid.

### Compound 1: 1-[(5-tert-butylsulfonyl-2-thienyl) sulfonyl]-4-chloro-3-[7-(5-chloropyrimidin-2-yl)-4, 7-diazaspiro [2.5]octan-4-yl]indazole

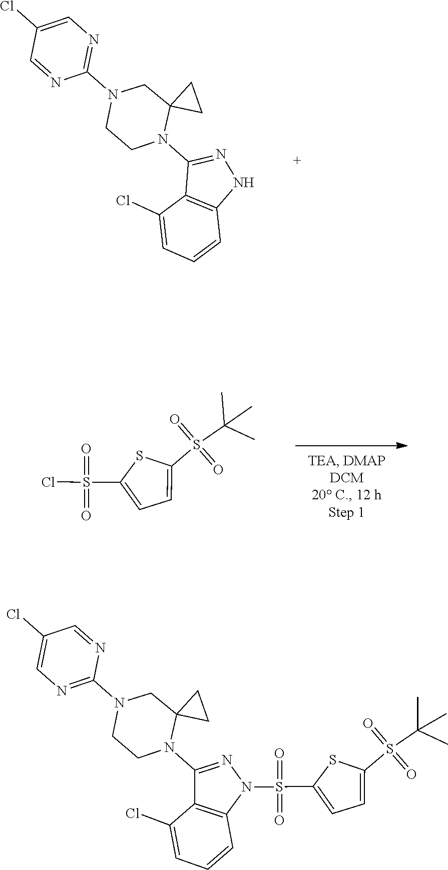 [SMILES: Clc1cnc(N2CCN(c3n[nH]c4cccc(Cl)c34)C3(CC3)C2)nc1 ; CC(C)(C)S(=O)(=O)c1ccc(S(=O)(=O)Cl)s1 ; CC(C)(C)S(=O)(=O)c1ccc(S(=O)(=O)n2nc(N3CCN(c4ncc(Cl)cn4)CC34CC4)c3c(Cl)cccc32)s1]

Step 1: To a solution of 4-chloro-3-[7-(5-chloropyrimidin-2-yl)-4,7-diazaspiro[2.5]octan-4-yl]-1H-indazole (30 mg, 80.0 μmol, 1 eq) and 5-tert-butylsulfonylthiophene-2-sulfonyl chloride (48.4 mg, 160 μmol, 2 eq) in DCM (2 mL) was added TEA (16.2 mg, 160 μmol, 22.3 μL, 2 eq) and DMAP (977 μg, 7.99 μmol, 0.1 eq). The mixture was stirred at 20° C. for 12 hours. LC-MS showed 4-chloro-3-[7-(5-chloropyrimidin-2-yl)-4, 7-diazaspiro [2.5]octan-4-yl]-1H-indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-HPLC (HCl condition; Method: column: Phenomenex Luna 80*30 mm*3 μm; mobile phase: [water(HCl)-ACN]; B %: 35%-65%, 8 min) to give desired 1-[(5-tert-butylsulfonyl-2-thienyl)sulfonyl]-4-chloro-3-[7-(5-chloropyrimidin-2-yl)-4,7-diazaspiro[2.5]octan-4-yl]indazole (10.4 mg, 15.4 μmol, 19.3% yield, 98.85% purity) as a white solid. ¹H NMR (400 MHz, DMSO-d₆) δ 8.45 (s, 2H), 8.04 (d, J=8.4 Hz, 1H), 7.85 (d, J=4.1 Hz, 1H), 7.75 (d, J=4.1 Hz, 1H), 7.70 (t, J=8.1 Hz, 1H), 7.54 (d, J=7.8 Hz, 1H), 4.00 (s, 2H), 3.82-3.64 (m, 2H), 3.60 (br s, 2H), 1.21 (s, 9H), 0.83 (s, 2H), 0.50 (s, 2H). HPLC: 98.85% (220 nm), 98.49% (215 nm), 99.53% (254 nm). MS (ESI): mass calcd. For C₂₅H₂₆C₁₂N₆O₄S₃ 640.0, m/z found 641.1 [M+H]⁺.

### Compound 2: 4-chloro-3-(7-(5-chloropyrimidin-2-yl)-4, 7-diazaspiro [2.5]octan-4-yl)-1-((4-(isopropylsulfonyl) phenyl) sulfonyl)-1H-indazole

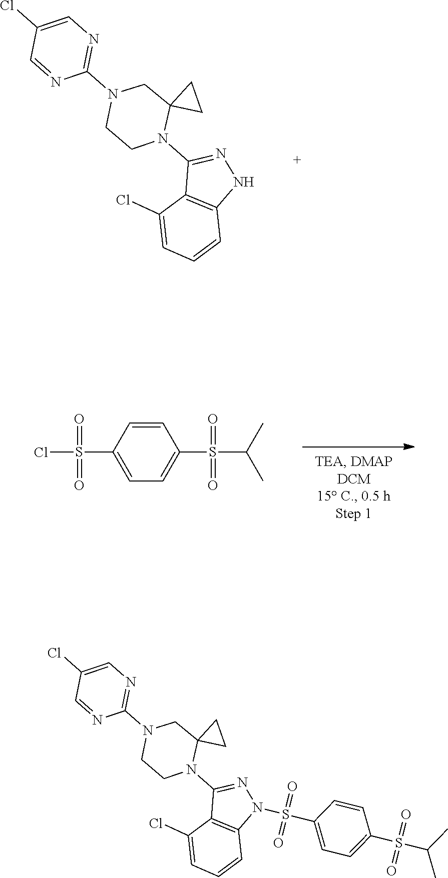 [SMILES: Clc1cnc(N2CCN(c3n[nH]c4cccc(Cl)c34)C3(CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ncc(Cl)cn4)CC34CC4)c3c(Cl)cccc32)cc1]

Step 1: To a solution of 4-chloro-3-[7-(5-chloropyrimidin-2-yl)-4,7-diazaspiro[2.5]octan-4-yl]-1H-indazole (30 mg, 80.0 μmol, 1 eq) and 4-isopropylsulfonylbenzenesulfonyl chloride (29.4 mg, 104 μmol, 1.3 eq) in DCM (1 mL) was added TEA (24.3 mg, 240 μmol, 33.4 μL, 3 eq) and DMAP (977 μg, 7.99 μmol, 0.1 eq). The mixture was stirred at 15° C. for 0.5 hour. LC-MS showed 4-chloro-3-[7-(5-chloropyrimidin-2-yl)-4, 7-diazaspiro [2.5]octan-4-yl]-1H-indazole was consumed completely and desired mass was detected. The reaction mixture was concentrated under reduced pressure to give a residue. The residue was purified by prep-HPLC (FA condition; column: Phenomenex Luna C18 75*30 mm*3 μm; mobile phase: [water (FA)-CAN]; B %: 40%-80%, 8 min) to give desired 4-chloro-3-(7-(5-chloropyrimidin-2-yl)-4, 7-diazaspiro [2.5]octan-4-yl)-1-((4-(isopropylsulfonyl) phenyl) sulfonyl)-1H-indazole (16.9 mg, 27.2 μmol, 34.0% yield, 100% purity) as a white solid. ¹H NMR (400 MHz, DMSO-d₆) δ 8.44 (s, 2H), 8.11-8.01 (m, 5H), 7.67 (t, J=8.1 Hz, 1H), 7.49 (d, J=7.7 Hz, 1H), 3.96 (s, 2H), 3.74-3.50 (m, 4H), 3.50-3.42 (m, 1H), 1.08 (d, J=6.9 Hz, 6H), 0.77 (s, 2H), 0.36 (br s, 2H). HPLC: 100.00% (220 nm), 99.82% (215 nm), 100.00% (254 nm). MS (ESI): mass calcd. For C₂₆H₂₆C₁₂N₆O₄S₂ 620.08, m/z found 621.2 [M+H]⁺.

### Compound 3: 4-chloro-6-fluoro-3-(7-(5-fluoropyrimidin-2-yl)-4, 7-diazaspiro [2.5]octan-4-yl)-1-((4-(isopropylsulfonyl) phenyl) sulfonyl)-1H-indazole

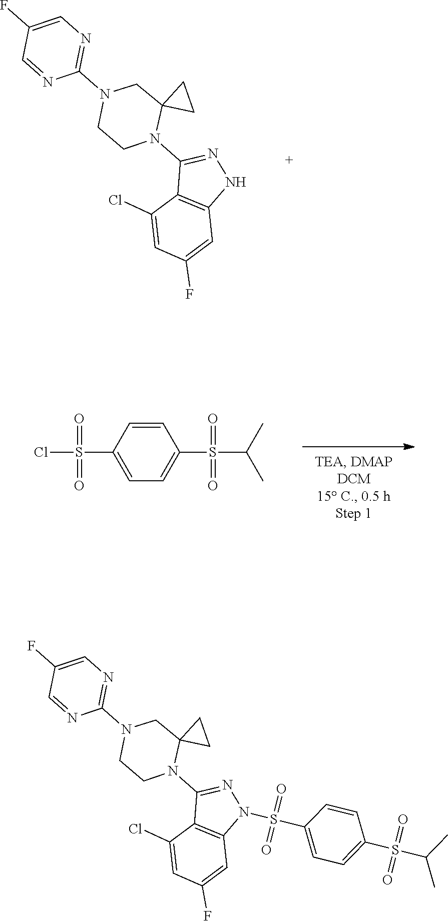 [SMILES: Fc1cnc(N2CCN(c3n[nH]c4cc(F)cc(Cl)c34)C3(CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cn4)CC34CC4)c3c(Cl)cc(F)cc32)cc1]

Step 1: To a solution of 4-chloro-6-fluoro-3-[7-(5-fluoropyrimidin-2-yl)-4,7-diazaspiro[2.5]octan-4-yl]-1H-indazole (30 mg, 79.6 μmol, 1 eq) and 4-isopropylsulfonylbenzenesulfonyl chloride (29.3 mg, 104 μmol, 1.3 eq) in DCM (1 mL) was added TEA (24.2 mg, 239 μmol, 33.3 μL, 3 eq) and DMAP (973 μg, 7.96 μmol, 0.1 eq). The mixture was stirred at 15° C. for 0.5 hour. LC-MS showed 4-chloro-6-fluoro-3-[7-(5-fluoropyrimidin-2-yl)-4, 7-diazaspiro [2.5]octan-4-yl]-1H-indazole was consumed completely and desired mass was detected. The reaction mixture was concentrated under reduced pressure to give a residue. The residue was purified by prep-HPLC (FA condition; column: Phenomenex Luna C18 75*30 mm*3 μm; mobile phase: [water(FA)-ACN]; B %: 40%-80%, 8 min) to give desired 4-chloro-6-fluoro-3-(7-(5-fluoropyrimidin-2-yl)-4,7-diazaspiro[2.5]octan-4-yl)-1-((4-(isopropylsulfonyl)phenyl)sulfonyl)-1H-indazole (13.6 mg, 21.8 μmol, 27.4% yield, 99.90% purity) as a white solid. ¹H NMR (400 MHz, DMSO-d₆) δ 8.47 (s, 2H), 8.16-8.10 (m, 2H), 8.06-8.01 (m, 2H), 7.86 (dd, J=2.1, 8.8 Hz, 1H), 7.58 (dd, J=2.1, 9.1 Hz, 1H), 3.90 (s, 2H), 3.52 (br s, 4H), 3.50-3.43 (m, 1H), 1.09 (d, J=6.8 Hz, 6H), 0.76 (s, 2H), 0.35 (br s, 2H). HPLC: 99.90% (220 nm), 100.00% (215 nm), 100.00% (254 nm). MS (ESI): mass calcd. For C₂₆H₂₅ClF₂N₆O₄S₂ 622.10, m/z found 623.2 [M+H]⁺.

### Compound 4: 4-chloro-3-[7-(5-chloropyrimidin-2-yl)-4, 7-diazaspiro [2.5]octan-4-yl]-1-(3-isopropylsulfonylphenyl) sulfonyl-indazole

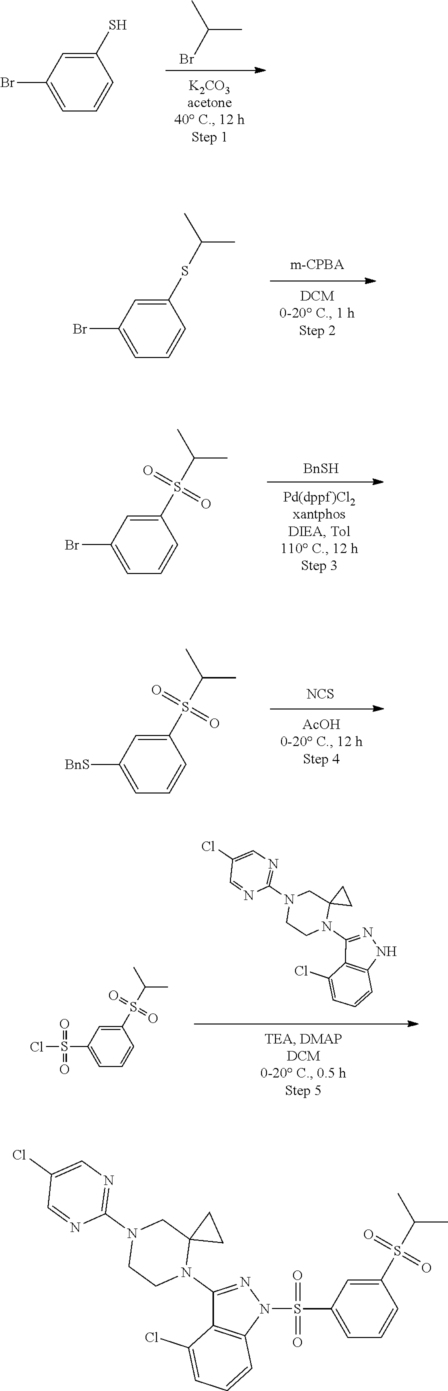

Step 1: A mixture of 3-bromobenzenethiol (1 g, 5.29 mmol, 546 μL, 1 eq), 2-bromopropane (1.30 g, 10.6 mmol, 993 μL, 2 eq), K₂CO₃ (2.56 g, 18.5 mmol, 3.5 eq) in acetone (10 mL) was degassed and purged with N₂ for 3 times, and then the mixture was stirred at 40° C. for 12 hours under the atmosphere of nitrogen. TLC (petroleum ether/ethyl acetate=10/1) indicated 3-bromobenzenethiol was consumed completely and one new spot formed. The reaction was clean according to TLC. The reaction mixture was added to water (20 mL), extracted with EtOAc (10 mL*3). The combined organic layers were washed with brine (20 mL) and dried over Na₂SO₄, filtered and concentrated under reduced pressure to give desired 1-bromo-3-isopropylsulfanyl-benzene (1.2 g, crude) as a yellow oil.

Step 2: To a solution of 1-bromo-3-isopropylsulfanyl-benzene (1.2 g, 5.19 mmol, 1 eq) in DCM (10 mL) was added m-CPBA (3.16 g, 15.6 mmol, 85% purity, 3 eq) at 0° C. The mixture was stirred at 20° C. for 1 hour. TLC (petroleum ether/ethyl acetate=3/1) indicated 1-bromo-3-isopropylsulfanyl-benzene was consumed completely and one new spot formed. Then it was partitioned between 30 mL of sat. Na₂SO₃ and 100 mL of DCM. The organic phase was separated, washed with 30 mL of sat. Na₂SO₃, 30 mL of brine, dried over Na₂SO₄, filtered and concentrated under reduced pressure to give desired 1-bromo-3-isopropylsulfonyl-benzene (2 g, crude) as a white solid.

Step 3: A mixture of phenylmethanethiol (1.04 g, 8.36 mmol, 980 μL, 1.1 eq), 1-bromo-3-isopropylsulfonyl-benzene (2 g, 7.60 mmol, 1 eq), DIEA (1.96 g, 15.2 mmol, 2.65 mL, 2 eq), Xantphos (440 mg, 760 μmol, 0.1 eq) and Pd(dppf)C₁₂ (139 mg, 190 μmol, 0.025 eq) in Tol. (20 mL) was degassed and purged with N₂ for 3 times, and then the mixture was stirred at 110° C. for 12 hours under the atmosphere of nitrogen. TLC (petroleum ether/ethyl acetate=3/1) indicated phenylmethanethiol was consumed completely and one new spot formed. The reaction mixture was concentrated under reduced pressure to remove Tol. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by column chromatography (SiO₂, petroleum ether/ethyl acetate=1/0 to 1/1) to give desired 1-benzylsulfanyl-3-isopropylsulfonyl-benzene (1.2 g, crude) as an orange oil.

Step 4: To a solution of 1-benzylsulfanyl-3-isopropylsulfonyl-benzene (1.2 g, 3.92 mmol, 1 eq) in AcOH (10 mL) and H₂O (2.5 mL) was added NCS (1.57 g, 11.8 mmol, 3 eq) at 0° C. The mixture was stirred at 20° C. for 12 hours. TLC (petroleum ether/ethyl acetate=3/1) showed 1-benzylsulfanyl-3-isopropylsulfonyl-benzene was consumed completely and one major new spot with larger polarity was detected. The reaction mixture was diluted with water 50 mL and extracted with EtOAc (50 mL*3). The combined organic layers were washed with brine (50 mL), dried over anhydrous Na₂SO₄, filtered and concentrated under reduced pressure to give a residue. The residue was purified by column chromatography (SiO₂, petroleum ether/ethyl acetate=1/0 to 0/1) to give desired 3-isopropylsulfonylbenzenesulfonyl chloride (600 mg, crude) as a white solid.

Step 5: To a solution of 4-chloro-3-[7-(5-chloropyrimidin-2-yl)-4,7-diazaspiro[2.5]octan-4-yl]-1H-indazole (50 mg, 133 μmol, 1 eq) and 3-isopropylsulfonylbenzenesulfonyl chloride (56.5 mg, 200 μmol, 1.5 eq) in DCM (2 mL) was added TEA (27.0 mg, 266 μmol, 37.01 μL, 2 eq) and DMAP (1.63 mg, 13.3 μmol, 0.1 eq) at 0° C. The mixture was stirred at 20° C. for 0.5 hour. LC-MS showed 4-chloro-3-[7-(5-chloropyrimidin-2-yl)-4, 7-diazaspiro [2.5]octan-4-yl]-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Phenomenex C18 75*30 mm*3 m; mobile phase: [water(NH₄HCO₃)-ACN]; B %: 60%-90%, 8 min) to give desired 4-chloro-3-[7-(5-chloropyrimidin-2-yl)-4,7-diazaspiro[2.5]octan-4-yl]-1-(3-isopropylsulfonylphenyl)sulfonyl-indazole (9.6 mg, 15.0 μmol, 11.28% yield, 97.32% purity) as a yellow solid. ¹H NMR (400 MHz, DMSO-d₆) δ 8.48 (s, 2H), 8.24 (br t, J=9.1 Hz, 2H), 8.16 (d, J=8.5 Hz, 1H), 8.10 (s, 1H), 7.93 (t, J=7.9 Hz, 1H), 7.72 (t, J=8.1 Hz, 1H), 7.53 (d, J=7.8 Hz, 1H), 4.01 (s, 2H), 3.71 (br s, 2H), 3.59 (br d, J=4.0 Hz, 2H), 3.52-3.44 (m, 1H), 1.01 (d, J=6.8 Hz, 6H), 0.83 (br s, 2H), 0.42 (br s, 2H). HPLC: 97.32% (220 nm), 96.74% (215 nm), 96.79% (254 nm). MS (ESI): mass calcd. For C₂₆H₂₆C₁₂N₆O₄S₂ 620.08, m/z found 621.1[M+H]⁺.

### Compound 5: 4-chloro-3-[4-(5-chloropyrimidin-2-yl)-2-cyclopropyl-piperazin-1-yl]-1-[(5-isopropylsulfonyl-2-thienyl) sulfonyl]indazole

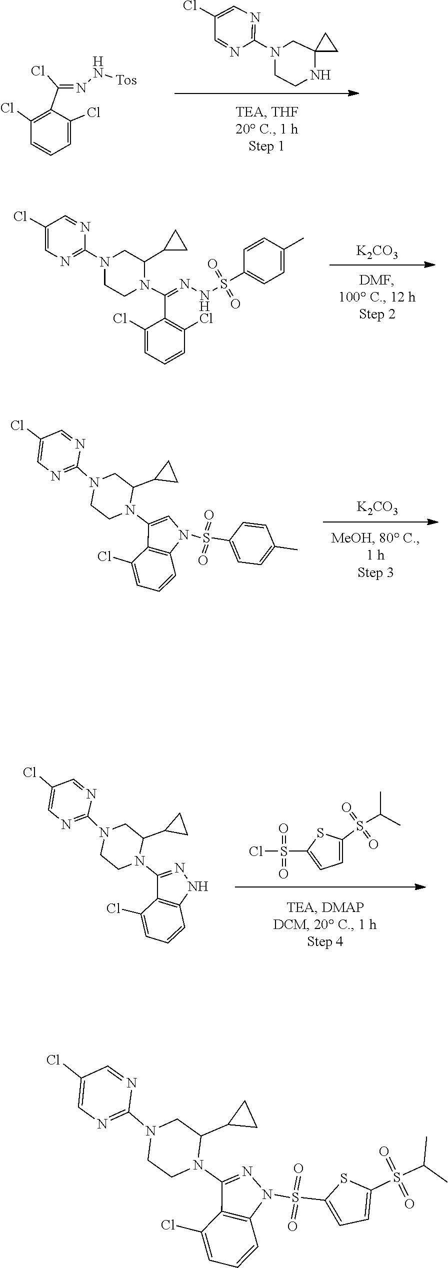

Step 1: To a solution of (1Z)-2,6-dichloro-N-(p-tolylsulfonyl)benzohydrazonoyl chloride (308 mg, 818 μmol, 1.5 eq) and 5-chloro-2-(3-cyclopropylpiperazin-1-yl) pyrimidine (150 mg, 545 μmol, 1 eq, HCl) in THF (8 mL) was added TEA (138 mg, 1.36 mmol, 190 μL, 2.5 eq). The mixture was stirred at 20° C. for 1 hour. LC-MS showed (1Z)-2, 6-dichloro-N-(p-tolylsulfonyl) benzohydrazonoyl chloride (308 mg, 818 μmol, 1.5 eq) and 5-chloro-2-(3-cyclopropylpiperazin-1-yl) pyrimidine was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N—[(E)-[[4-(5-chloropyrimidin-2-yl)-2-cyclopropyl-piperazin-1-yl]-(2,6-dichlorophenyl)methylene]amino]-4-methyl-benzenesulfonamide (470 mg, crude) as a yellow solid. MS (ESI): mass calcd. For C₂₅H₂₅C₁₃N₆O₂S 578.08, m/z found 579.1 [M+H]⁺.

Step 2: To a solution of N—[(E)-[[4-(5-chloropyrimidin-2-yl)-2-cyclopropyl-piperazin-1-yl]-(2, 6-dichlorophenyl) methylene]amino]-4-methyl-benzenesulfonamide (470 mg, 810 μmol, 1 eq) in DMF (5 mL) was added K₂CO₃ (1.12 g, 8.10 mmol, 10 eq). The mixture was stirred at 100° C. for 12 hours. LC-MS showed N—[(E)-[[4-(5-chloropyrimidin-2-yl)-2-cyclopropyl-piperazin-1-yl]-(2, 6-dichlorophenyl) methylene]amino]-4-methyl-benzenesulfonamide was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired 4-chloro-3-[4-(5-chloropyrimidin-2-yl)-2-cyclopropyl-piperazin-1-yl]-1-(p-tolylsulfonyl)indazole (700 mg, crude) as a yellow solid. MS (ESI): mass calcd. For C₂₅H₂₄C₁₂N₆O₂S 542.11, m/z found 543.1 [M+H]⁺.

Step 3: To a solution of 4-chloro-3-[4-(5-chloropyrimidin-2-yl)-2-cyclopropyl-piperazin-1-yl]-1-(p-tolylsulfonyl) indazole (700 mg, 1.29 mmol, 1 eq) in MeOH (10 mL) was added K₂CO₃ (890 mg, 6.44 mmol, 5 eq). The mixture was stirred at 80° C. for 1 hour. LC-MS showed 4-chloro-3-[4-(5-chloropyrimidin-2-yl)-2-cyclopropyl-piperazin-1-yl]-1-(p-tolylsulfonyl) indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether/ethyl acetate=2/1) to give desired 4-chloro-3-[4-(5-chloropyrimidin-2-yl)-2-cyclopropyl-piperazin-1-yl]-1H-indazole (80 mg, 206 μmol, 16.0% yield) as a yellow solid. MS (ESI): mass calcd. For C₁₈H₁₈C₁₂N₆ 388.1 m/z found 389.1 [M+H]⁺.

Step 4: To a solution of 4-chloro-3-[4-(5-chloropyrimidin-2-yl)-2-cyclopropyl-piperazin-1-yl]-1H-indazole (30 mg, 77.1 μmol, 1 eq) and 5-isopropylsulfonylthiophene-2-sulfonyl chloride (33.4 mg, 116 μmol, 1.5 eq) in DCM (1 mL, 94.4% purity) was added TEA (15.6 mg, 154 μmol, 21.5 μL, 2 eq) and DMAP (942 μg, 7.71 μmol, 0.1 eq). The mixture was stirred at 20° C. for 1 hour. LC-MS showed 4-chloro-3-[4-(5-chloropyrimidin-2-yl)-2-cyclopropyl-piperazin-1-yl]-1H-indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-HPLC (basic condition; Method:column: Waters Xbridge Prep OBD C18 150*40 mm*10 μm; mobile phase: [water(NH₄HCO₃)-ACN]; B %: 60%-90%, 8 min to give desired 4-chloro-3-[4-(5-chloropyrimidin-2-yl)-2-cyclopropyl-piperazin-1-yl]-1-[(5-isopropylsulfonyl-2-thienyl)sulfonyl]indazole (12.3 mg, 18.1 μmol, 23.48% yield, 94.38% purity) as a white solid. ¹H NMR (400 MHz, DMSO-d₆) δ=8.51-8.44 (m, 2H), 8.09-8.03 (m, 1H), 7.89 (d, J=4.1 Hz, 1H), 7.79-7.75 (m, 1H), 7.73-7.67 (m, 1H), 7.57-7.52 (m, 1H), 4.57-4.48 (m, 2H), 3.72-3.56 (m, 4H), 3.30-3.21 (m, 2H), 1.22 (br d, J=6.8 Hz, 1H), 1.14 (d, J=6.8 Hz, 6H), 0.28-0.17 (m, 1H), 0.06 (qd, J=4.8, 9.4 Hz, 1H), −0.09-−0.18 (m, 1H), −0.76 (qd, J=4.9, 9.3 Hz, 1H). HPLC: 94.38% (220 nm), 90.95% (215 nm), 99.64% (254 nm). MS (ESI): mass calcd. For C₂₅H₂₆C₁₂N₆O₄S₃ 640.06, m/z found 641.1 [M+H]⁺.

### Compound 6: 4-chloro-1-[(5-isopropylsulfonyl-2-thienyl) sulfonyl]-3-[7-(2, 2, 2-trifluoroethyl)-4, 7-diazaspiro [2.5]octan-4-yl]indazole

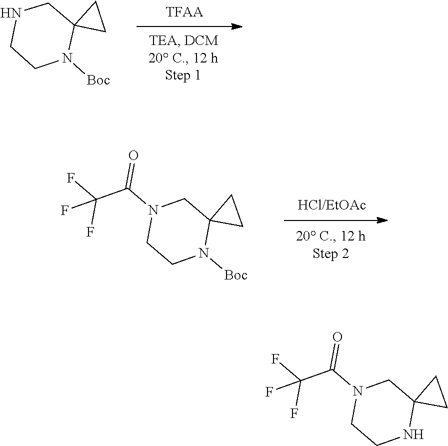 [SMILES: CC(C)(C)OC(=O)N1CCNCC12CC2 ; "TFAA" ; CC(C)(C)OC(=O)N1CCN(C(=O)C(F)(F)F)CC12CC2 ; O=C(N1CCNC2(CC2)C1)C(F)(F)F]

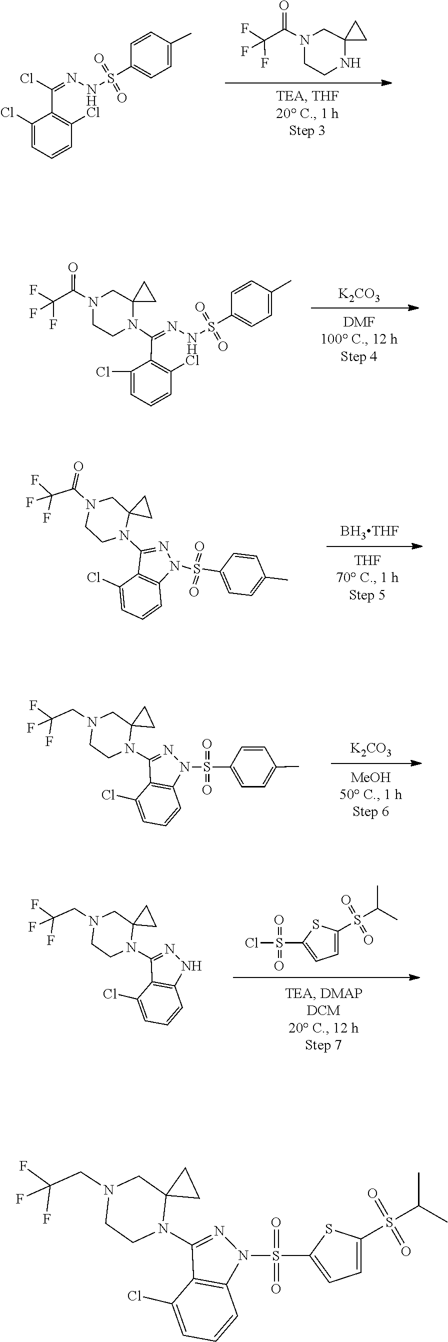

Step 1: To a solution of tert-butyl 4, 7-diazaspiro [2.5]octane-4-carboxylate (2 g, 9.42 mmol, 1 eq in DCM (20 mL) was added TFAA (2.37 g, 11.3 mmol, 1.57 mL, 1.2 eq and TEA (2.86 g, 28.3 mmol, 3.93 mL, 3 @q. The mixture was stirred at 20° C. for 12 hours. LC-MS showed tert-butyl 4, 7-diazaspiro [2.5]octane-4-carboxylate was consumed completely and one main peak with desired mass was detected. The reaction mixture was diluted with H₂O (50 mL) and extracted with DCM (100 mL). The combined organic layers were washed with brine (40 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜40% EtOAc/petroleum ether gradient @50 mL/min) to give desired tert-butyl 7-(2, 2, 2-trifluoroacetyl)-4, 7-diazaspiro [2.5]octane-4-carboxylate (2.4 g, 7.78 mmol, 82.6% yield) as a white solid. MS (ESI): mass calcd. For C₁₃H₁₉F₃N₂O₃ 308.13 m/z found 209.1 [M+H−100]⁺.

Step 2: To a solution of tert-butyl 7-(2, 2, 2-trifluoroacetyl)-4, 7-diazaspiro [2.5]octane-4-carboxylate (2.4 g, 7.78 mmol, 1 eq) in HCl/EtOAc (50 mL). The mixture was stirred at 20° C. for 12 hours. LC-MS showed tert-butyl 7-(2, 2, 2-trifluoroacetyl)-4, 7-diazaspiro [2.5]octane-4-carboxylate was consumed completely and one main peak with desired mass was detected. The reaction mixture was concentrated under reduced pressure to remove EtOAc to give desired 1-(4, 7-diazaspiro [2.5]octan-7-yl)-2, 2, 2-trifluoro-ethanone (1.6 g, crude) as a white solid. MS (ESI): mass calcd. For C₈H₁₁F₃N₂O 208.08 m/z found 209.1 [M+H]⁺.

Step 3: To a solution of 1-(4, 7-diazaspiro [2.5]octan-7-yl)-2, 2, 2-trifluoro-ethanone (1.09 g, 4.45 mmol, 1.2 eq, HCl) in THF (5 mL) was added TEA (1.13 g, 11.1 mmol, 1.55 mL, 3 eq). The mixture was stirred at 0° C. for 20 minutes. Then (1E)-2,6-dichloro-N-(p-tolylsulfonyl)benzohydrazonoyl chloride (1.4 g, 3.71 mmol, 1 eq) was added to the mixture at 0° C. The mixture was stirred at 20° C. for 40 minutes. LC-MS showed (1E)-2, 6-dichloro-N-(p-tolylsulfonyl) benzohydrazonoyl chloride was consumed completely and one main peak with desired mass was detected. The reaction mixture was diluted with H₂O (20 mL) and extracted with EtOAc (60 mL). The combined organic layers were washed with brine (40 mL), dried over anhydrous Na₂SO₄, filtered and concentrated under reduced pressure to give desired N—[(E)-[(2,6-dichlorophenyl)-[7-(2,2,2-trifluoroacetyl)-4,7-diazaspiro[2.5]octan-4-yl]methylene]amino]-4-methyl-benzenesulfonamide (2 g, crude) as a yellow oil. MS (ESI): mass calcd. For C₂₂H₂₁Cl₂F₃N₄O₃S 548.07 m/z found 549.1 [M+H]⁺.

Step 4: To a solution of N—[(E)-[(2,6-dichlorophenyl)-[7-(2,2,2-trifluoroacetyl)-4,7-diazaspiro[2.5]octan-4-yl]methylene]amino]-4-methyl-benzenesulfonamide (2 g, 3.64 mmol, 1 eq) in DMF (20 mL) was added K₂CO₃ (5.03 g, 36.4 mmol, 10 eq). The mixture was stirred at 80° C. for 12 hours. LC-MS showed N—[(E)-[(2,6-dichlorophenyl)-[7-(2,2,2-trifluoroacetyl)-4,7-diazaspiro[2.5]octan-4-yl]methylene]amino]-4-methyl-benzenesulfonamide was consumed completely and one main peak with desired mass was detected. The reaction mixture was diluted with H₂O (50 mL) and extracted with EtOAc (150 mL). The combined organic layers were washed with brine (100 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜25% EtOAc/Petroleum ether gradient @50 mL/min) to give desired 1-[4-[4-chloro-1-(p-tolylsulfonyl)indazol-3-yl]-4,7-diazaspiro[2.5]octan-7-yl]-2,2,2-trifluoro-ethanone (860 mg, 1.68 mmol, 46.1% yield) as a pale yellow oil. MS (ESI): mass calcd. For C₂₂H₂₀ClF₃N₄O₃S 512.09 m/z found 513.1 [M+H]⁺.

Step 5: To a solution of 1-[4-[4-chloro-1-(p-tolylsulfonyl)indazol-3-yl]-4,7-diazaspiro[2.5]octan-7-yl]-2,2,2-trifluoro-ethanone (430 mg, 838 μmol, 1 eq) in THF (5 mL) was added BH₃·THF (1 M, 5 mL, 5.96 eq) dropwise. The mixture was stirred at 70° C. for 2 hours. LC-MS showed 1-[4-[4-chloro-1-(p-tolylsulfonyl) indazol-3-yl]-4, 7-diazaspiro [2.5]octan-7-yl]-2, 2, 2-trifluoro-ethanone was consumed completely and one main peak with desired mass was detected. The reaction mixture was quenched by addition MeOH (10 mL) at 0° C., and stirred at 0° C. for 30 minutes, and then concentrated under reduced pressure to give a residue. The residue was purified by prep-TLC (SiO₂, petroleum ether/EtOAc=4/1) to give desired 4-chloro-1-(p-tolylsulfonyl)-3-[7-(2, 2, 2-trifluoroethyl)-4, 7-diazaspiro [2.5]octan-4-yl]indazole (300 mg, 601 μmol, 71.7% yield) as a colourless oil. MS (ESI): mass calcd. For C₂₂H₂₂ClF₃N₄O₂S 498.11 m/z found 499.2 [M+H]⁺.

Step 6: To a solution of 4-chloro-1-(p-tolylsulfonyl)-3-[7-(2, 2, 2-trifluoroethyl)-4, 7-diazaspiro [2.5]octan-4-yl]indazole (150 mg, 301 μmol, 1 eq) in MeOH (5 mL) was added K₂CO₃ (208 mg, 1.50 mmol, 5 eq). The mixture was stirred at 50° C. for 1 hour. LC-MS showed 4-chloro-1-(p-tolylsulfonyl)-3-[7-(2, 2, 2-trifluoroethyl)-4, 7-diazaspiro [2.5]octan-4-yl]indazole was consumed completely and one main peak with desired mass was detected. The reaction mixture was concentrated under reduced pressure to remove MeOH. The residue was diluted with H₂O (5 mL) and extracted with EtOAc (30 mL). The combined organic layers were washed with brine (20 mL), dried over anhydrous Na₂SO₄, filtered and concentrated under reduced pressure to give a residue. The residue was purified by prep-TLC (SiO₂, petroleum ether/EtOAc=2/1) to give desired 4-chloro-3-[7-(2, 2, 2-trifluoroethyl)-4, 7-diazaspiro [2.5]octan-4-yl]-1H-indazole (80 mg, 232 μmol, 77.2% yield) as a colorless oil. MS (ESI): mass calcd. For C₁₅H₁₆ClF₃N₄ 344.10 m/z found 345.0 [M+H]⁺.

Step 7: To a solution of 4-chloro-3-[7-(2, 2, 2-trifluoroethyl)-4, 7-diazaspiro [2.5]octan-4-yl]-1H-indazole (30 mg, 87.0 μmol, 1 eq) and 5-isopropylsulfonylthiophene-2-sulfonyl chloride (50.3 mg, 174 μmol, 2 eq) in DCM (2 mL) was added TEA (26.4 mg, 261 μmol, 36.3 μL, 3 eq) and DMAP (1.06 mg, 8.70 μmol, 0.1 eq). The mixture was stirred at 20° C. for 12 hours. LC-MS showed 4-chloro-3-[7-(2, 2, 2-trifluoroethyl)-4, 7-diazaspiro [2.5]octan-4-yl]-1H-indazole was consumed completely and one main peak with desired mass was detected. The reaction mixture was diluted with H₂O (5 mL) and extracted with DCM (10 mL). The combined organic layers were washed with brine (10 mL), dried over anhydrous Na₂SO₄, filtered and concentrated under reduced pressure to give a residue. The residue was purified by prep-HPLC (neutral condition: column: Waters Xbridge BEH C18 100*30 mm*10 μm; mobile phase: [water (NH₄HCO₃)-ACN]; B %: 55%-85%, 8 min) to give desired 4-chloro-1-[(5-isopropylsulfonyl-2-thienyl)sulfonyl]-3-[7-(2,2,2-trifluoroethyl)-4,7-diazaspiro[2.5]octan-4-yl]indazole (20 mg, 33.5 μmol, 38.5% yield, 99.9% purity) as a pale yellow solid. ¹H NMR (400 MHz, DMSO-d₆) δ 8.01 (d, J=8.51 Hz, 1H) 7.77-7.84 (m, 2H) 7.66 (t, J=8.07 Hz, 1H) 7.51 (d, J=7.63 Hz, 1H) 3.61 (dt, J=13.51, 6.63 Hz, 1H) 3.52 (br t, J=4.44 Hz, 2H) 3.37 (br s, 4H) 3.19 (q, J=10.21 Hz, 2H) 1.15 (d, J=6.88 Hz, 6H) 0.73 (br s, 2H) 0.47 (br s, 2H). HPLC: 99.963% (220 nm), 100.00% (215 nm), 100.00% (254 nm). MS (ESI): mass calcd. For C₂₂H₂₄ClF₃N₄O₄S₃ 596.06 m/z found 597.1 [M+H]⁺.

### Compound 7: 4-[4-chloro-1-[(5-isopropylsulfonyl-2-thienyl) sulfonyl]indazol-3-yl]-7-oxa-4-azaspiro [2.5]octane

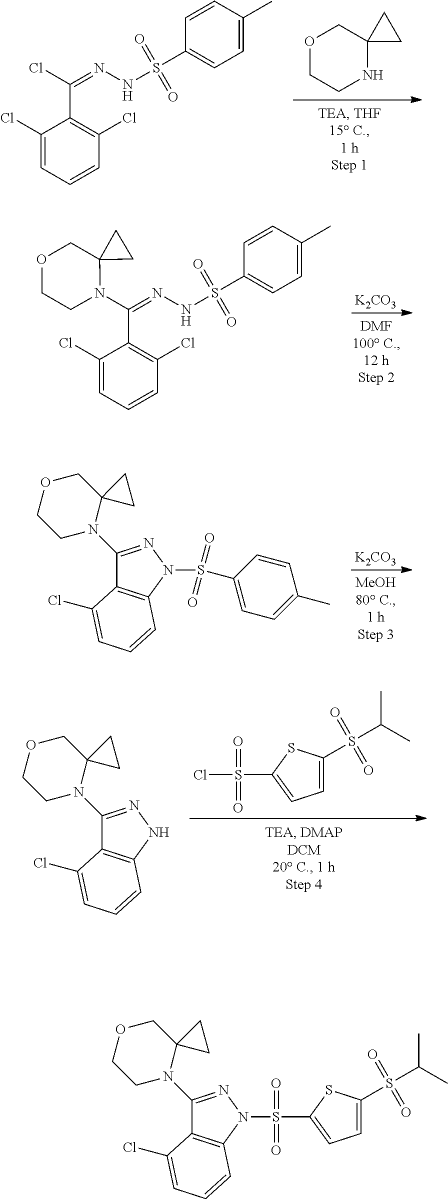

Step 1: To the solution of (1E)-2,6-dichloro-N-(p-tolylsulfonyl)benzohydrazonoyl chloride (252 mg, 668 μmol, 1 eq) in THF (3 mL) was added TEA (676 mg, 6.68 mmol, 930 μL, 10 eq) at 15° C. and the solution was stirred at 15° C. for 0.5 hour. To the reaction mixture was added 7-oxa-4-azaspiro [2.5]octane (100 mg, 668 μmol, 1 eq, HCl) at 15° C. and the solution was stirred at 15° C. for 0.5 hour. LCMS showed (1E)-2, 6-dichloro-N-(p-tolylsulfonyl) benzohydrazonoyl chloride was consumed completely and desired mass. The reaction mixture was concentrated to give desired N—[(E)-[(2, 6-dichlorophenyl)-(7-oxa-4-azaspiro [2.5]octan-4-yl) methylene]amino]-4-methyl-benzenesulfonamide (0.303 g, crude) as a yellow solid. MS (ESI): mass calcd. For C₂₀H₂₁C₁₂N₃O₃S 453.07, m/z found 454.1 [M+H]⁺.

Step 2: The mixture of N—[(E)-[(2, 6-dichlorophenyl)-(7-oxa-4-azaspiro [2.5]octan-4-yl) methylene]amino]-4-methyl-benzenesulfonamide (0.303 g, 667 μmol, 1 eq) and K₂CO₃ (922 mg, 6.67 mmol, 10 eq) in DMF (5 mL) was stirred at 100° C. for 12 hours. LCMS showed N—[(E)-[(2, 6-dichlorophenyl)-(7-oxa-4-azaspiro [2.5]octan-4-yl) methylene]amino]-4-methyl-benzenesulfonamide was consumed completely and desired mass was detected. The reaction was concentrated to get a residue. The residue was added water (5 mL) and extracted with MTBE (2*20 mL). The combined organics were concentrated to give desired 4-[4-chloro-1-(p-tolylsulfonyl) indazol-3-yl]-7-oxa-4-azaspiro [2.5]octane (278 mg, crude) as a yellow solid. MS (ESI): mass calcd. For C₂₅H₂₄C₁₂N₆O₂S 542.11, m/z found 543.1 [M+H]⁺.

Step 3: To a solution of 4-[4-chloro-1-(p-tolylsulfonyl) indazol-3-yl]-7-oxa-4-azaspiro[2.5]octane (278 mg, 665 μmol, 1 eq) in MeOH (5 mL) was added K₂CO₃ (460 mg, 3.33 mmol, 5 eq). The mixture was stirred at 80° C. for 1 hour. LC-MS showed 4-[4-chloro-1-(p-tolylsulfonyl) indazol-3-yl]-7-oxa-4-azaspiro [2.5]octane was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether:EtOAc=2:1) to give desired 4-(4-chloro-1H-indazol-3-yl)-7-oxa-4-azaspiro [2.5]octane (90 mg, 341 μmol, 51.30% yield) as a yellow solid. MS (ESI): mass calcd. For C₁₃H₁₄ClN₃O 263.08 m/z found 264.1 [M+H]⁺.

Step 4: To a solution of 4-(4-chloro-1H-indazol-3-yl)-7-oxa-4-azaspiro[2.5]octane (30 mg, 114 μmol, 1 eq) and 5-isopropylsulfonylthiophene-2-sulfonyl chloride (49.3 mg, 171 μmol, 1.5 eq) in DCM (1 mL) was added TEA (23.0 mg, 228 μmol, 31.7 μL, 2 eq) and DMAP (1.39 mg, 11.4 μmol, 0.1 eq). The mixture was stirred at 20° C. for 1 hour. LC-MS showed 4-(4-chloro-1H-indazol-3-yl)-7-oxa-4-azaspiro [2.5]octane was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-HPLC (FA condition; Method:column: Phenomenex Luna C18 75*30 mm*3 μm; mobile phase: [water(FA)-ACN]; B %: 35%-75%, 8 min) to give desired 4-[4-chloro-1-[(5-isopropylsulfonyl-2-thienyl)sulfonyl]indazol-3-yl]-7-oxa-4-azaspiro[2.5]octane (6.9 mg, 22.8 μmol, 20.02% yield, 97.96% purity) as a white solid. ¹H NMR (400 MHz, DMSO-d₆) δ=8.01 (d, J=8.4 Hz, 1H), 7.83 (d, J=4.1 Hz, 1H), 7.79 (s, 1H), 7.70-7.64 (m, 1H), 7.51 (d, J=7.6 Hz, 1H), 3.80-3.70 (m, 1H), 3.64-3.57 (m, 1H), 3.57 (br s, 4H), 1.15 (d, J=6.8 Hz, 6H), 0.95-0.94 (m, 1H), 0.81-0.75 (m, 2H), 0.50-0.43 (m, 2H). HPLC: 97.96% (220 nm), 97.88% (215 nm), 100.0% (254 nm). MS (ESI): mass calcd. For C₂₀H₂₂ClN₃O₅S₃ 515.04, m/z found 516.1 [M+H]⁺.

### Compound 8: 4-[4-chloro-1-[(5-isopropylsulfonyl-2-thienyl) sulfonyl]indazol-3-yl]-4-azaspiro [2.5]octan-7-one

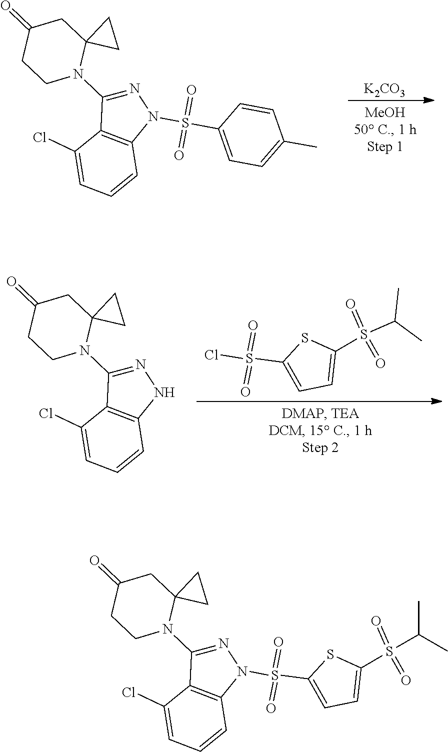 [SMILES: Cc1ccc(S(=O)(=O)n2nc(N3CCC(=O)CC34CC4)c3c(Cl)cccc32)cc1 ; "K2CO3" ; O=C1CCN(c2n[nH]c3cccc(Cl)c23)C2(CC2)C1 ; CC(C)S(=O)(=O)c1ccc(S(=O)(=O)Cl)s1 ; CC(C)S(=O)(=O)c1ccc(S(=O)(=O)n2nc(N3CCC(=O)CC34CC4)c3c(Cl)cccc32)s1]

Step 1: The mixture of 4-[4-chloro-1-(p-tolylsulfonyl) indazol-3-yl]-4-azaspiro [2.5]octan-7-one (30 mg, 69.8 μmol, 1 eq) and K₂CO₃ (96.4 mg, 698 μmol, 10 eq) in MeOH (2 mL) was stirred at 50° C. for 1 hour. TLC showed 4-chloro-3-(7, 7-difluoro-4-azaspiro [2.5]octan-4-yl)-1-(p-tolylsulfonyl) indazole was consumed completely. The reaction was concentrated to get a residue. The residue was added water (5 mL) and extracted with MTBE (2*20 mL). The combined organics were concentrated to give desired 4-(4-chloro-1H-indazol-3-yl)-4-azaspiro [2.5]octan-7-one (10 mg, crude) as a yellow oil.

Step 2: To the solution of 4-(4-chloro-1H-indazol-3-yl)-4-azaspiro[2.5]octan-7-one (10 mg, 36.3 μmol, 1 eq), DMAP (443 μg, 3.63 μmol, 0.1 eq) and TEA (18.4 mg, 181 μmol, 5 eq) in DCM (2 mL) was added 5-isopropylsulfonylthiophene-2-sulfonyl chloride (15.7 mg, 54.4 μmol, 1.5 eq) and the solution was stirred at 15° C. for 1 hour. LCMS showed 4-(4-chloro-1H-indazol-3-yl)-4-azaspiro [2.5]octan-7-one was consumed completely and desired mass was detected. The reaction was concentrated to get a residue. The residue was purified by prep-HPLC (column: Waters Xbridge BEH C18 100*30 mm*10 um; mobile phase: [water(NH₄HCO₃)-ACN]; B %: 50%-80%, 8 min) to give desired 4-[4-chloro-1-[(5-isopropylsulfonyl-2-thienyl)sulfonyl]indazol-3-yl]-4-azaspiro[2.5]octan-7-one (2.0 mg, 3.58 μmol, 9.88% yield, 94.59% purity) as a white solid. ¹H NMR (400 MHz, DMSO-d₆) δ 8.08-8.14 (m, 1H), 7.93 (d, 1H), 7.85 (d, 1H), 7.72-7.81 (m, 1H), 7.61 (d, 1H), 3.84 (br m, 2H), 3.67 (m, 1H), 2.74 (m, 1H), 2.31-2.44 (m, 3H), 1.21 (d, 6H), 0.72-0.80 (m, 2H), 0.52-0.65 (m, 2H). HPLC: 94.59% (220 nm), 91.74% (215 nm), 100.0% (254 nm). MS (ESI): mass calcd. For C₂₁H₂₂N₃S₃O₅Cl 527.04 m/z found 528.0 [M+H]⁺.

### Compound 9: 1-[4-[4-chloro-1-[(5-isopropylsulfonyl-2-thienyl) sulfonyl]indazol-3-yl]-4, 7-diazaspiro [2.5]octan-7-yl]-2, 2, 2-trifluoro-ethanone

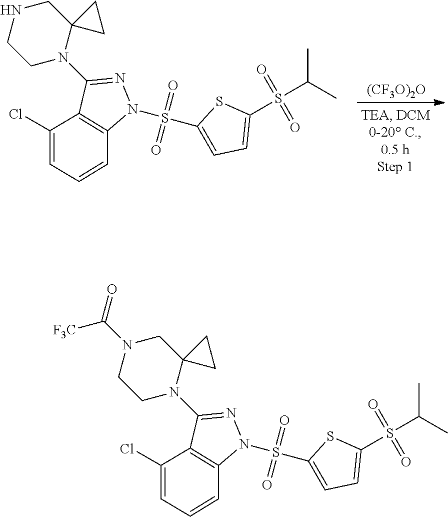

Step 1: To a solution of 4-chloro-3-(4,7-diazaspiro[2.5]octan-4-yl)-1-[(5-isopropylsulfonyl-2-thienyl)sulfonyl]indazole (30 mg, 58.2 μmol, 1 eq) in DCM (2 mL) was added TEA (11.8 mg, 116 μmol, 16.2 μL, 2 eq) and (2,2,2-trifluoroacetyl) 2,2,2-trifluoroacetate (14.7 mg, 69.9 μmol, 9.72 μL, 1.2 eq) at 0° C. The mixture was stirred at 20° C. for 0.5 hour. LC-MS showed 4-chloro-3-(4, 7-diazaspiro [2.5]octan-4-yl)-1-[(5-isopropylsulfonyl-2-thienyl) sulfonyl]indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water(NH₄HCO₃)-ACN]; B %: 60%-90%, 8 min) to give desired 1-[4-[4-chloro-1-[(5-isopropylsulfonyl-2-thienyl)sulfonyl]indazol-3-yl]-4,7-diazaspiro[2.5]octan-7-yl]-2,2,2-trifluoro-ethanone (3.3 mg, 5.31 μmol, 9.12% yield, 98.38% purity) as a white solid. ¹H NMR (400 MHz, DMSO-d₆) δ 8.10 (d, J=8.4 Hz, 1H), 7.93 (dd, J=4.2, 5.3 Hz, 1H), 7.85 (dd, J=1.4, 4.1 Hz, 1H), 7.76 (t, J=8.1 Hz, 1H), 7.60 (d, J=7.8 Hz, 1H), 3.99-3.81 (m, 2H), 3.75-3.66 (m, 3H), 3.65-3.55 (m, 2H), 1.21 (d, J=6.9 Hz, 6H), 0.95 (br d, J=16.0 Hz, 2H), 0.60 (br d, J=8.5 Hz, 2H). HPLC: 98.38% (220 nm), 98.45% (215 nm), 98.86% (254 nm). MS (ESI): mass calcd. For C₂₂H₂₂ClF₃N₄O₅S₃ 610.04, m/z found 611.1 [M+H]⁺.

### Compound 10: 4-chloro-3-[7-(5-fluoropyrimidin-2-yl)-4, 7-diazaspiro [2.5]octan-4-yl]-1-(4-isopropylsulfonylphenyl) sulfonyl-indazole

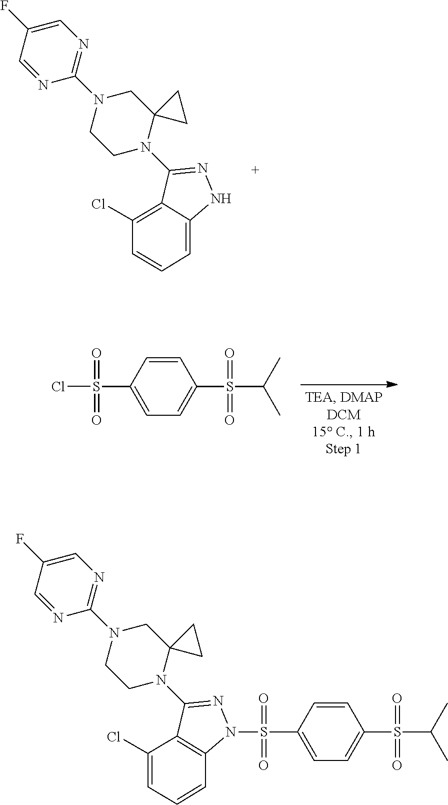 [SMILES: Fc1cnc(N2CCN(c3n[nH]c4cccc(Cl)c34)C3(CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cn4)CC34CC4)c3c(Cl)cccc32)cc1]

Step 1: To a solution of 4-chloro-3-[7-(5-fluoropyrimidin-2-yl)-4, 7-diazaspiro [2.5]octan-4-yl]-1H-indazole (30 mg, 83.6 μmol, 1 eq) and 4-isopropylsulfonylbenzenesulfonyl chloride (35.5 mg, 125 μmol, 1.5 eq) in DCM (1 mL) was added TEA (21.2 mg, 209 μmol, 29.1 μL, 2.5 eq) and DMAP (1.02 mg, 8.36 μmol, 0.1 eq). The mixture was stirred at 15° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was concentrated under reduced pressure to give a residue. The residue was purified by prep-HPLC (neutral condition; column: Waters Xbridge BEH C18 100*30 mm*10 μm; mobile phase: [water(NH₄HCO₃)-ACN]; B %: 60%-95%, 8 min) to give desired 4-chloro-3-[7-(5-fluoropyrimidin-2-yl)-4, 7-diazaspiro [2.5]octan-4-yl]-1-(4-isopropylsulfonylphenyl) sulfonyl-indazole (24.2 mg, 39.2 μmol, 46.8% yield, 97.98% purity) as a white solid. ¹H NMR (400 MHz, DMSO-d₆) δ 8.44 (s, 2H), 8.06 (d, J=8.6 Hz, 1H), 8.05-8.01 (m, 2H), 8.01-7.97 (m, 2H), 7.64 (t, J=8.1 Hz, 1H), 7.46 (d, J=7.9 Hz, 1H), 3.89 (s, 2H), 3.66-3.46 (m, 4H), 3.46-3.39 (m, 1H), 1.05 (d, J=6.8 Hz, 6H), 0.74 (br s, 2H), 0.32 (br s, 2H). HPLC: 97.98% (220 nm), 98.01% (215 nm), 98.17% (254 nm). MS (ESI): mass calcd. For C₂₆H₂₆ClFN₆O₄S₂ 604.11 m/z found 605.1 [M+H]⁺.

### Compound 11: 4-chloro-3-(7-fluoro-4-azaspiro [2.5]octan-4-yl)-1-[(5-isopropylsulfonyl-2-thienyl) sulfonyl]indazole

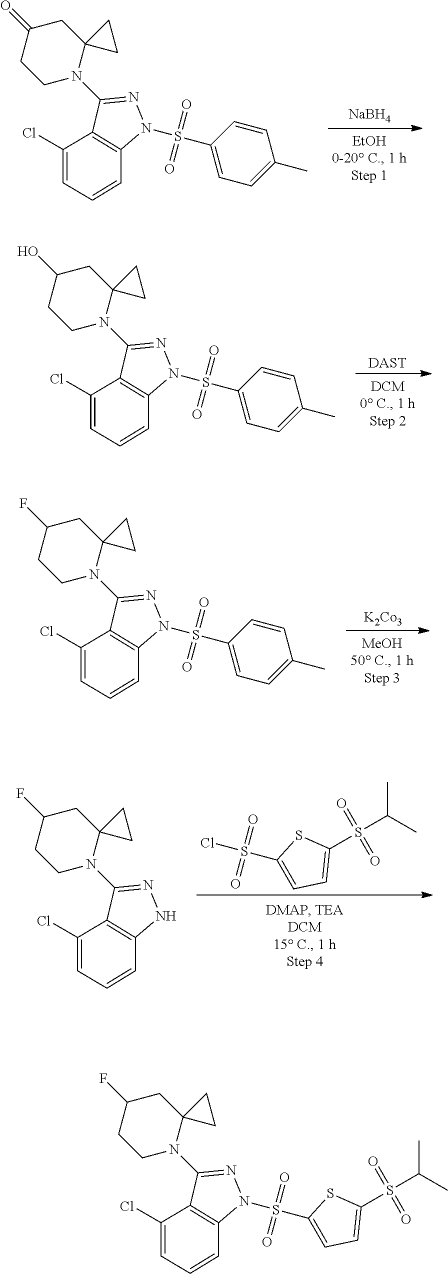

Step 1: To the solution of 4-[4-chloro-1-(p-tolylsulfonyl) indazol-3-yl]-4-azaspiro [2.5]octan-7-one (100 mg, 233 μmol, 1 eq) in EtOH (2 mL) was added NaBH₄ (70 mg, 1.85 mmol, 7.95 eq) at 0° C. and the solution was stirred at 20° C. for 1 hour. TLC showed 4-[4-chloro-1-(p-tolylsulfonyl) indazol-3-yl]-4-azaspiro [2.5]octan-7-one was consumed completely. The reaction was quenched with NH₄Cl solution (2 mL) and stirred for 15 minutes. The mixture was concentrated to get a residue. The residue was added water (2 mL) and extracted with EtOAc (2*5 mL). The combined organics were dried over anhydrous sodium sulfate and concentrated to give desired 4-[4-chloro-1-(p-tolylsulfonyl) indazol-3-yl]-4-azaspiro [2.5]octan-7-ol (100 mg, crude) as a yellow solid.

Step 2: To the solution of 4-[4-chloro-1-(p-tolylsulfonyl)indazol-3-yl]-4-azaspiro[2.5]octan-7-ol (80 mg, 185 μmol, 1 eq) in DCM (2 mL) was added DAST (59.7 mg, 370 μmol, 2 eq) at 0° C. and the solution was stirred at 0° C. for 1 hour. LCMS showed 4-[4-chloro-1-(p-tolylsulfonyl) indazol-3-yl]-4-azaspiro [2.5]octan-7-ol remained and desired mass was detected. The reaction was quenched with MeOH (1 mL) and concentrated to give desired 4-chloro-3-(7-fluoro-4-azaspiro [2.5]octan-4-yl)-1-(p-tolylsulfonyl)indazole (80 mg, crude) as a yellow oil. MS (ESI): mass calcd. For C₂₁H₂₁N₃ClSO₂F 433.1, m/z found 434.2 [M+H]⁺.

Step 3: The mixture of 4-chloro-3-(7-fluoro-4-azaspiro [2.5]octan-4-yl)-1-(p-tolylsulfonyl)indazole (96.0 mg, 221 μmol, 1 eq) and K₂CO₃ (305 mg, 2.21 mmol, 10 eq) in MeOH (2 mL) was stirred at 50° C. for 1 hour. LCMS showed 4-chloro-3-(7-fluoro-4-azaspiro [2.5]octan-4-yl)-1-(p-tolylsulfonyl)indazole remained and desired mass was detected. The reaction was concentrated to get a residue. The residue was added water (5 mL) and extracted with MTBE (2*20 mL). The combined organics were concentrated to get a residue. The residue was purified by prep-TLC (SiO₂, petroleum ether/ethyl acetate=2/1) to give desired 4-chloro-3-(7-fluoro-4-azaspiro [2.5]octan-4-yl)-1H-indazole (30 mg, crude) as a yellow oil. MS (ESI): mass calcd. For C₁₄H₁₅N₃ClF 279.09, m/z found 280.1 [M+H]⁺.

Step 4: To the solution of 4-chloro-3-(7-fluoro-4-azaspiro[2.5]octan-4-yl)-1H-indazole (30 mg, 107 μmol, 1 eq), DMAP (1.31 mg, 10.7 μmol, 0.1 eq) and TEA (54.3 mg, 536 μmol, 5 eq) in DCM (2 mL) was added 5-isopropylsulfonylthiophene-2-sulfonyl chloride (31.0 mg, 107 μmol, 1 eq) and the solution was stirred at 15° C. for 1 hour. LCMS showed 4-chloro-3-(7, 7-difluoro-4-azaspiro [2.5]octan-4-yl)-1H-indazole remained and desired mass was detected. The reaction was concentrated to get a residue. The residue was prep-HPLC (column: Waters Xbridge BEH C18 100*30 mm*10 μm; mobile phase: [water (NH₄HCO₃)-ACN]; B %: 60%-90%, 8 min) to give desired 4-chloro-3-(7-fluoro-4-azaspiro[2.5]octan-4-yl)-1-[(5-isopropylsulfonyl-2-thienyl)sulfonyl]indazole (3.3 mg, 5.61 μmol, 5.23% yield, 90.49% purity) as a white solid. ¹H NMR (DMSO-d₆) δ 8.03 (d, 1H), 7.83 (d, 1H), 7.75-7.80 (m, 1H), 7.68 (m, 1H), 7.52 (d, 1H), 4.85-5.07 (m, 1H), 3.56-3.72 (m, 4H), 1.70-1.85 (m, 2H), 1.49-1.63 (m, 1H), 1.16 (d, 6H), 0.60-0.77 (m, 2H), 0.27-0.52 (m, 2H) HPLC: 90.49% (220 nm), 88.01% (215 nm), 98.41% (254 nm). MS (ESI): mass calcd. For C₂₁H₂₃N₃S₃O₄ClF 531.05 m/z found 532.0 [M+H]⁺.

### Compound 12: 4-chloro-3-(7, 7-difluoro-4-azaspiro [2.5]octan-4-yl)-1-[(5-isopropylsulfonyl-2-thienyl) sulfonyl]indazole

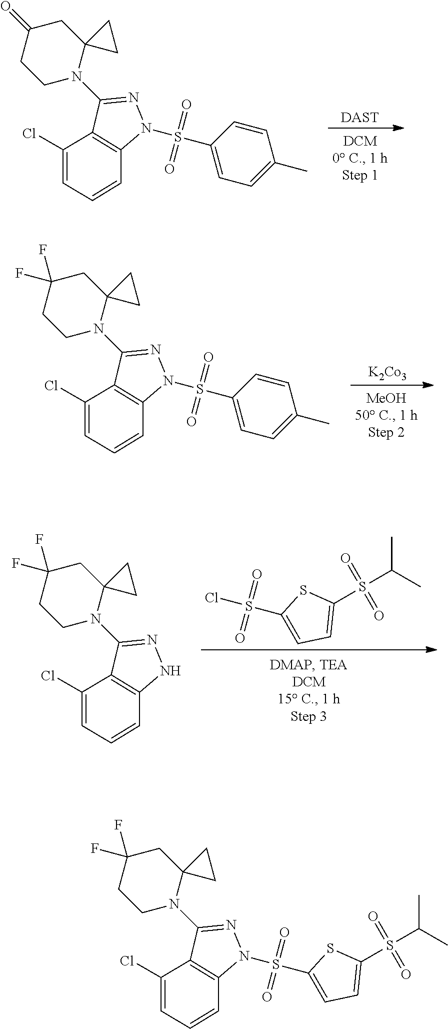

Step 1: To the solution of 4-[4-chloro-1-(p-tolylsulfonyl)indazol-3-yl]-4-azaspiro[2.5]octan-7-one (100 mg, 233 μmol, 1 eq) in DCM (2 mL) was added DAST (75.0 mg, 465 μmol, 2 eq) at 0° C. and the solution was stirred at 0° C. for 1 hour. LCMS showed 4-[4-chloro-1-(p-tolylsulfonyl) indazol-3-yl]-4-azaspiro [2.5]octan-7-one remained and desired mass was detected. The reaction was quenched with MeOH (1 mL) and concentrated to give desired 4-chloro-3-(7, 7-difluoro-4-azaspiro [2.5]octan-4-yl)-1-(p-tolylsulfonyl) indazole (105 mg, crude) as a yellow oil. MS (ESI): mass calcd. For C₂₁H₂₀N₃ClSO₂F₂ 451.09 m/z found 452.1 [M+H]⁺.

Step 2: The mixture of 4-chloro-3-(7, 7-difluoro-4-azaspiro [2.5]octan-4-yl)-1-(p-tolylsulfonyl) indazole (105 mg, 233 μmol, 1 eq) and K₂CO₃ (321 mg, 2.32 mmol, 10 eq) in MeOH (2 mL) was stirred at 50° C. for 1 hour. LCMS showed 4-chloro-3-(7, 7-difluoro-4-azaspiro [2.5]octan-4-yl)-1-(p-tolylsulfonyl) indazole was consumed completely and desired mass was detected. The reaction was concentrated to get a residue. The residue was added water (5 mL) and extracted with MTBE (2*20 mL). The combined organics were concentrated to get a residue. The residue was purified by prep-TLC (SiO₂, petroleum ether/ethyl acetate=2/1) to give desired 4-chloro-3-(7, 7-difluoro-4-azaspiro [2.5]octan-4-yl)-1H-indazole (10 mg, crude) was a yellow oil. MS (ESI): mass calcd. For C₁₄H₁₄N₃ClF₂ 297.08 m/z found 298.0 [M+H]⁺.

Step 3: To the solution of 4-chloro-3-(7,7-difluoro-4-azaspiro[2.5]octan-4-yl)-1H-indazole (10 mg, 33.6 μmol, 1 eq), DMAP (410 μg, 3.36 μmol, 0.1 eq) and TEA (17.0 mg, 168 μmol, 5 eq) in DCM (2 mL) was added 5-isopropylsulfonylthiophene-2-sulfonyl chloride (14.6 mg, 50.4 μmol, 1.5 eq) and the solution was stirred at 15° C. for 1 hour. LCMS showed 4-chloro-3-(7, 7-difluoro-4-azaspiro [2.5]octan-4-yl)-1H-indazole remained and desired mass was detected. The reaction was concentrated to get a residue. The residue was purified by prep-HPLC (column: Waters Xbridge BEH C18 100*30 mm*10 μm; mobile phase: [water(NH₄HCO₃)-ACN]; B %: 65%-95%, 8 min) to give desired 4-chloro-3-(7,7-difluoro-4-azaspiro[2.5]octan-4-yl)-1-[(5-isopropylsulfonyl-2-thienyl)sulfonyl]indazole (2.0 mg, 3.40 μmol, 10.1% yield, 93.46% purity) as a white solid. ¹H NMR (DMSO-d₆) δ: 8.10 (d, 1H), 7.92 (d, 1H), 7.85 (d, 1H), 7.75 (m, 1H), 7.59 (d, 1H), 3.63-3.72 (m, 4H), 1.95 (br m, 2H), 1.82 (m, 1H), 1.22 (d, 6H), 0.81 (s, 2H), 0.54 (br s, 2H). HPLC: 93.46% (220 nm), 92.14% (215 nm), 98.78% (254 nm). MS (ESI): mass calcd. For C₂₁H₂₂N₃S₃O₄ClF₂ 549.04 m/z found 550.0 [M+H]⁺.

### Compound 13: 4-[4-chloro-1-[(5-isopropylsulfonyl-2-thienyl) sulfonyl]indazol-3-yl]-N, N-dimethyl-4-azaspiro [2.5]octan-7-amine

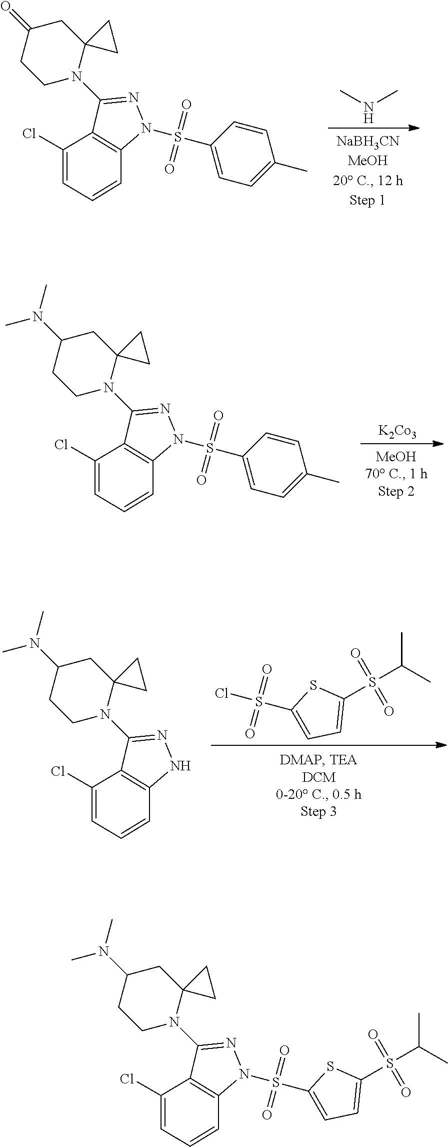

Step 1: To a solution of 4-[4-chloro-1-(p-tolylsulfonyl)indazol-3-yl]-4-azaspiro[2.5]octan-7-one (200 mg, 465 μmol, 1 eq) and N-methylmethanamine (168 mg, 3.72 mmol, 189 μL, 8 eq) in DCE (2 mL) was added sodium triacetoxyborohydride (148 mg, 698 μmol, 1.5 eq) and Acetic acid (0.02 mL). The mixture was stirred at 20° C. for 12 hours. LC-MS showed 4-[4-chloro-1-(p-tolylsulfonyl) indazol-3-yl]-4-azaspiro [2.5]octan-7-one was consumed completely and desired mass was detected. The reaction mixture was added to sat.NaHCO₃ aq. (20 mL), extracted with EtOAc (10 mL*3). The combined organic layers were washed with brine 20 mL and dried over Na₂SO₄, filtered and concentrated under reduced pressure to give desired 4-[4-chloro-1-(p-tolylsulfonyl) indazol-3-yl]-N, N-dimethyl-4-azaspiro [2.5]octan-7-amine (300 mg, crude) as a yellow oil. MS (ESI): mass calcd. For C₂₃H₂₇ClN₄O₂S 458.15, m/z found 459.3[M+H]⁺.

Step 2: To a solution of 4-[4-chloro-1-(p-tolylsulfonyl) indazol-3-yl]-N, N-dimethyl-4-azaspiro [2.5]octan-7-amine (300 mg, 654 μmol, 1 eq) in MeOH (10 mL) was added K₂CO₃ (452 mg, 3.27 mmol, 5 eq). The mixture was stirred at 70° C. for 1 hour. LC-MS showed 4-[4-chloro-1-(p-tolylsulfonyl) indazol-3-yl]-N, N-dimethyl-4-azaspiro [2.5]octan-7-amine was consumed completely and desired compound was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Phenomenex C18 75*30 mm*3 m; mobile phase: [water (NH₄HCO₃)-ACN]; B %: 30%-60%, 8 min) to give desired 4-(4-chloro-1H-indazol-3-yl)-N, N-dimethyl-4-azaspiro [2.5]octan-7-amine (50 mg, crude) as a yellow oil. MS (ESI): mass calcd. For C₁₆H₂₁ClN₄ 304.15, m/z found 305.1 [M+H]⁺.

Step 3: To a solution of 5-isopropylsulfonylthiophene-2-sulfonyl chloride (42.6 mg, 148 mol, 1.5 eq) and 4-(4-chloro-1H-indazol-3-yl)-N,N-dimethyl-4-azaspiro[2.5]octan-7-amine (30 mg, 98.4 μmol, 1 eq) in DCM (2 mL) was added TEA (19.9 mg, 197 μmol, 27.4 μL, 2 eq) and DMAP (1.20 mg, 9.84 μmol, 0.1 eq) at 0° C. The mixture was stirred at 20° C. for 0.5 hour. LC-MS showed 4-(4-chloro-1H-indazol-3-yl)-N, N-dimethyl-4-azaspiro [2.5]octan-7-amine was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water (NH₄HCO₃)-ACN]; B %: 45%-75%, 8 min) to give desired 4-[4-chloro-1-[(5-isopropylsulfonyl-2-thienyl)sulfonyl]indazol-3-yl]-N,N-dimethyl-4-azaspiro[2.5]octan-7-amine (23.4 mg, 40.7 μmol, 41.3% yield, 96.80% purity) as a white solid. ¹HNMR (400 MHz, DMSO-d₆) δ 8.02 (d, J=8.3 Hz, 1H), 7.85 (d, J=4.2 Hz, 1H), 7.79 (d, J=4.0 Hz, 1H), 7.68 (t, J=8.1 Hz, 1H), 7.52 (d, J=7.7 Hz, 1H), 3.73-3.57 (m, 2H), 3.32-3.25 (m, 1H), 2.49-2.40 (m, 2H), 2.19 (s, 6H), 1.72 (br d, J=12.7 Hz, 1H), 1.28-1.18 (m, 2H), 1.16 (dd, J=3.7, 6.7 Hz, 6H), 0.79-0.66 (m, 1H), 0.56-0.44 (m, 2H), 0.33 (td, J=5.8, 9.9 Hz, 1H). HPLC: 96.80% (220 nm), 97.70% (215 nm), 96.97% (254 nm). MS (ESI): mass calcd. For C₂₃H₂₉ClN₄O₄S₃ 556.10, m/z found 557.1 [M+H]⁺.

### Compound 14: 4-chloro-1-[(5-isopropylsulfonyl-2-thienyl) sulfonyl]-3-[7-(trifluoromethylsulfonyl)-4, 7-diazaspiro [2.5]octan-4-yl]indazole

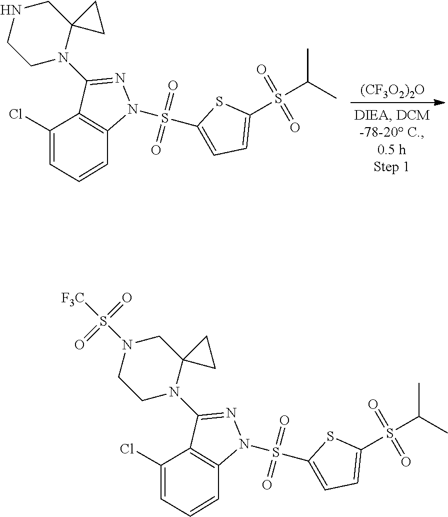

Step 1: To a solution of 4-chloro-3-(4,7-diazaspiro[2.5]octan-4-yl)-1-[(5-isopropylsulfonyl-2-thienyl)sulfonyl]indazole (30 mg, 58.2 μmol, 1 eq) in DCM (2 mL) was added DIEA (45.2 mg, 349 μmol, 60.9 μL, 6 eq) andtrifluoromethylsulfonyl trifluoromethanesulfonate (65.7 mg, 233 μmol, 38.4 μL, 4 eq) at −78° C. The mixture was stirred at 20° C. for 0.5 hour. LC-MS showed 4-chloro-3-(4, 7-diazaspiro [2.5]octan-4-yl)-1-[(5-isopropylsulfonyl-2-thienyl) sulfonyl]indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (FA condition; column: Phenomenex C18 75*30 mm*3 μm; mobile phase: [water(FA)-ACN]; B %: 55%-80%, 8 min) to give desired 4-chloro-1-[(5-isopropylsulfonyl-2-thienyl)sulfonyl]-3-[7-(trifluoromethylsulfonyl)-4,7-diazaspiro[2.5]octan-4-yl]indazole (3.1 mg, 4.79 μmol, 8.23% yield, 100.00% purity) as a pale yellow solid. ¹H NMR (400 MHz, DMSO-d₆) δ 8.04 (d, J=8.5 Hz, 1H), 7.88 (d, J=4.1 Hz, 1H), 7.80 (d, J=4.1 Hz, 1H), 7.70 (t, J=8.1 Hz, 1H), 7.54 (d, J=7.6 Hz, 1H), 3.89-3.69 (m, 2H), 3.68-3.56 (m, 3H), 3.54-3.41 (m, 2H), 1.17 (d, J=6.8 Hz, 6H), 0.95 (br s, 2H), 0.59 (br s, 2H). HPLC: 90.85% (220 nm), 100.00% (215 nm), 100.00% (254 nm). MS (ESI): mass calcd. For C₂₁H₂₂ClF₃N₄O₆S₄ 646.01, m/z found 647.1 [M+H]⁺.

### Compound 15: 3-(7-(5-chloropyrimidin-2-yl)-4, 7-diazaspiro [2.5]octan-4-yl)-4-fluoro-1-((4-(isopropylsulfonyl) phenyl) sulfonyl)-1H-indazole

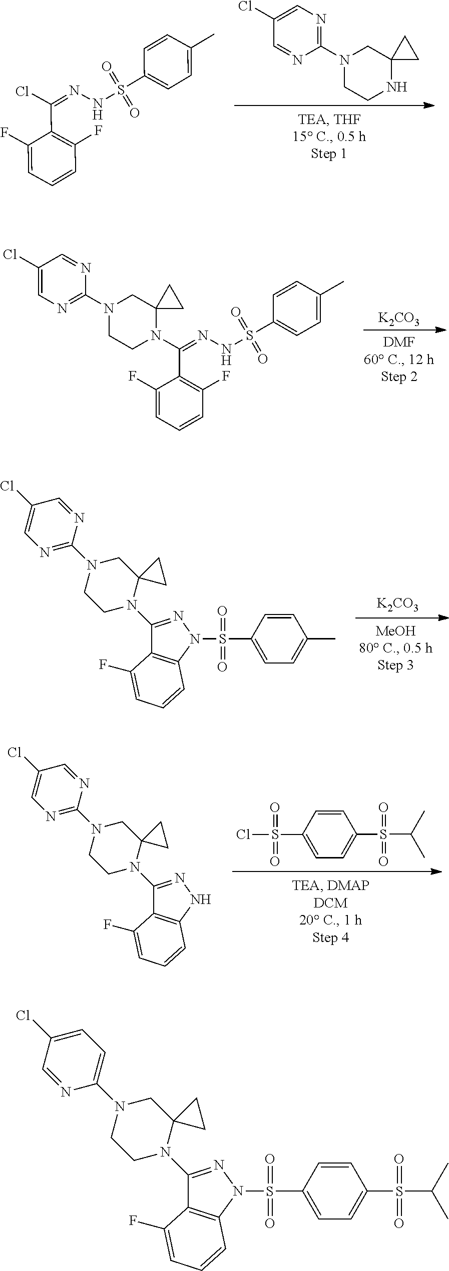

Step 1: To a solution of (1Z)-2,6-difluoro-N-(p-tolylsulfonyl)benzohydrazonoyl chloride (264 mg, 766 μmol, 1 eq) in THF (5 mL) was added TEA (194 mg, 1.91 mmol, 266 μL, 2.5 eq) and 7-(5-chloropyrimidin-2-yl)-4,7-diazaspiro[2.5]octane (200 mg, 766 μmol, 1 eq, HCl). The mixture was stirred at 15° C. for 0.5 hour. LC-MS showed (1Z)-2,6-difluoro-N-(p-tolylsulfonyl)benzohydrazonoyl chloride was consumed completely and one main peak with desired mass was detected. Then it was separated between water (20 mL) and ethyl acetate (15 mL*3). The organic phase was separated, washed with brine (20 mL), dried over Na₂SO₄, filtered and concentrated under reduced pressure to give desired (E)-N′-((7-(5-chloropyrimidin-2-yl)-4,7-diazaspiro[2.5]octan-4-yl)(2,6-difluorophenyl)methylene)-4-methylbenzenesulfonohydrazide (400 mg, crude) as a brown oil. MS (ESI): mass calcd. For C₂₄H₂₃ClF₂N₆O₂S 532.13, m/z found 533.2 [M+H]⁺.

Step 2: To a solution of N—[(E)-[[7-(5-chloropyrimidin-2-yl)-4, 7-diazaspiro [2.5]octan-4-yl]-(2, 6-difluorophenyl) methylene]amino]-4-methyl-benzenesulfonamide (400 mg, 750 μmol, 1 eq) in DMF (10 mL) was added K₂CO₃ (1.04 g, 7.50 mmol, 10 eq). The mixture was stirred at 60° C. for 12 hours. LC-MS showed N—[(E)-[[7-(5-chloropyrimidin-2-yl)-4, 7-diazaspiro [2.5]octan-4-yl]-(2, 6-difluorophenyl) methylene]amino]-4-methyl-benzenesulfonamide was consumed completely and a new spot. The reaction was concentrated to get desired 3-(7-(5-chloropyrimidin-2-yl)-4,7-diazaspiro[2.5]octan-4-yl)-4-fluoro-1-tosyl-1H-indazole (400 mg, crude) as a brown solid. MS (ESI): mass calcd. For C₂₄H₂₂ClFN₆O₂S 512.12, m/z found 513.3 [M+H]⁺.

Step 3: To a solution of 3-[7-(5-chloropyrimidin-2-yl)-4, 7-diazaspiro [2.5]octan-4-yl]-4-fluoro-1-(p-tolylsulfonyl) indazole (400 mg, 780 μmol, 1 eq) in MeOH (20 mL) was added K₂CO₃ (539 mg, 3.90 mmol, 5 eq). The mixture was stirred at 80° C. for 0.5 hour. TLC showed 3-[7-(5-chloropyrimidin-2-yl)-4, 7-diazaspiro [2.5]octan-4-yl]-4-fluoro-1-(p-tolylsulfonyl) indazole was consumed completely and a new spot. The reaction mixture was added to water (20 mL), the aqueous phase was extracted with EtOAc (15 mL*3). The organic layer was dried over Na₂SO₄, filtered and the filtrate was concentrated to give a residue. The residue was purified by prep-TLC (SiO₂, petroleum ether/Ethyl acetate=2/1) to give desired 3-(7-(5-chloropyrimidin-2-yl)-4,7-diazaspiro[2.5]octan-4-yl)-4-fluoro-1H-indazole (85 mg, 237 μmol, 30.38% yield) as a brown oil.

Step 4: To a solution of 3-[7-(5-chloropyrimidin-2-yl)-4,7-diazaspiro[2.5]octan-4-yl]-4-fluoro-1H-indazole (40 mg, 111 μmol, 1 eq) and 4-isopropylsulfonylbenzenesulfonyl chloride (47.3 mg, 167 μmol, 1.5 eq) in DCM (1 mL) was added TEA (28.2 mg, 279 μmol, 38.8 μL, 2.5 eq) and DMAP (1.36 mg, 11.2 μmol, 0.1 eq). The mixture was stirred at 15° C. for 1 hour. LC-MS showed 3-[7-(5-chloropyrimidin-2-yl)-4, 7-diazaspiro [2.5]octan-4-yl]-4-fluoro-1H-indazole was consumed completely and desired mass was detected. The reaction mixture was concentrated under reduced pressure to give a residue. The residue was purified by prep-HPLC (TFA condition; column: Phenomenex Luna C18 75*30 mm*3 μm; mobile phase: [water(TFA)-ACN]; B %: 55%-85%, 8 min) to give desired 3-(7-(5-chloropyrimidin-2-yl)-4,7-diazaspiro[2.5]octan-4-yl)-4-fluoro-1-((4-(isopropylsulfonyl)phenyl)sulfonyl)-1H-indazole (18.5 mg, 30.3 μmol, 27.15% yield, 99.00% purity) as a white solid. ¹H NMR (400 MHz, DMSO-d₆) δ 8.44 (s, 2H), 8.09-8.01 (m, 4H), 7.96 (d, J=8.3 Hz, 1H), 7.73 (dt, J=5.1, 8.2 Hz, 1H), 7.26 (dd, J=8.1, 10.6 Hz, 1H), 3.93 (s, 2H), 3.64 (br s, 4H), 3.46-3.42 (m, 1H), 1.07 (d, J=6.8 Hz, 6H), 0.88 (br s, 2H), 0.51 (s, 2H). HPLC: 99.00% (220 nm), 99.85% (215 nm), 100.00% (254 nm). MS (ESI): mass calcd. For C₂₆H₂₆ClFN₆O₄S₂ 604.11 m/z found 605.2 [M+H]⁺.

### Compound 16: 4-fluoro-3-(7-(5-fluoropyrimidin-2-yl)-4, 7-diazaspiro [2.5]octan-4-yl)-1-((4-(isopropylsulfonyl) phenyl) sulfonyl)-1H-indazole

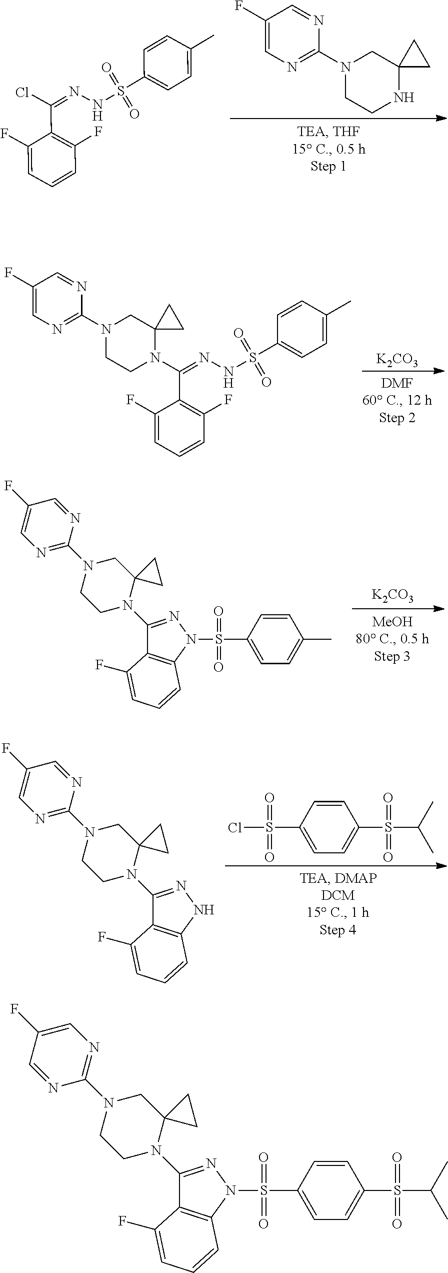

Step 1: To a solution of (1Z)-2,6-difluoro-N-(p-tolylsulfonyl)benzohydrazonoyl chloride (282 mg, 817 μmol, 1 eq) in THF (5 mL) was added TEA (207 mg, 2.04 mmol, 284 μL, 2.5 eq) and 7-(5-fluoropyrimidin-2-yl)-4,7-diazaspiro[2.5]octane (200 mg, 817 μmol, 1 eq, HCl). The mixture was stirred at 15° C. for 0.5 hour. LC-MS showed (1Z)-2, 6-difluoro-N-(p-tolylsulfonyl) benzohydrazonoyl chloride was consumed completely and one main peak with desired mass was detected. Then it was separated between water (20 mL) and ethyl acetate (15 mL*3). The organic phase was separated, washed with brine (20 mL), dried over Na₂SO₄, filtered and concentrated under reduced pressure to give desired (E)-N′-((2,6-difluorophenyl)(7-(5-fluoropyrimidin-2-yl)-4,7-diazaspiro[2.5]octan-4-yl)methylene)-4-methylbenzenesulfonohydrazide (400 mg, crude) as a brown oil. MS (ESI): mass calcd. For C₂₄H₂₃F₃N₆O₂S 516.16, m/z found 517.1 [M+H]⁺.

Step 2: To a solution of N—[(E)-[(2, 6-difluorophenyl)-[7-(5-fluoropyrimidin-2-yl)-4, 7-diazaspiro [2.5]octan-4-yl]methylene]amino]-4-methyl-benzenesulfonamide (400 mg, 774 μmol, 1 eq) in DMF (10 mL) was added K₂CO₃ (1.07 g, 7.74 mmol, 10 eq). The mixture was stirred at 60° C. for 12 hours. LC-MS showed N—[(E)-[(2, 6-difluorophenyl)-[7-(5-fluoropyrimidin-2-yl)-4, 7-diazaspiro [2.5]octan-4-yl]methylene]amino]-4-methyl-benzenesulfonamide was consumed completely and a new spot. The reaction was concentrated to get desired 4-fluoro-3-(7-(5-fluoropyrimidin-2-yl)-4,7-diazaspiro[2.5]octan-4-yl)-1-tosyl-1H-indazole (400 mg, crude) as a brown solid. MS (ESI): mass calcd. For C₂₄H₂₂F₂N₆O₂S 496.15, m/z found 497.2 [M+H]⁺.

Step 3: To a solution of 4-fluoro-3-[7-(5-fluoropyrimidin-2-yl)-4, 7-diazaspiro [2.5]octan-4-yl]-1-(p-tolylsulfonyl) indazole (400 mg, 806 μmol, 1 eq) in MeOH (20 mL) was added K₂CO₃ (557 mg, 4.03 mmol, 5 eq). The mixture was stirred at 80° C. for 0.5 hour. TLC showed 4-fluoro-3-[7-(5-fluoropyrimidin-2-yl)-4, 7-diazaspiro [2.5]octan-4-yl]-1-(p-tolylsulfonyl) indazole was consumed completely and a new spot. The reaction mixture was added to water (20 mL), the aqueous phase was extracted with EtOAc (15 mL*3). The organic layer was dried over Na₂SO₄, filtered and the filtrate was concentrated to give a residue. The residue was purified by prep-TLC (SiO₂, petroleum ether/Ethyl acetate=2/1) to give desired 4-fluoro-3-(7-(5-fluoropyrimidin-2-yl)-4, 7-diazaspiro [2.5]octan-4-yl)-1H-indazole (85 mg, 248 μmol, 30.8% yield) as a brown oil.

Step 4: To a solution of 4-fluoro-3-[7-(5-fluoropyrimidin-2-yl)-4,7-diazaspiro[2.5]octan-4-yl]-1H-indazole (40 mg, 117 μmol, 1 eq) and 4-isopropylsulfonylbenzenesulfonyl chloride (49.6 mg, 175 μmol, 1.5 eq) in DCM (1 mL) was added TEA (29.6 mg, 292 μmol, 40.7 μL, 2.5 eq) and DMAP (1.43 mg, 11.7 μmol, 0.1 eq). The mixture was stirred at 15° C. for 1 hour. LC-MS showed 4-fluoro-3-[7-(5-fluoropyrimidin-2-yl)-4, 7-diazaspiro [2.5]octan-4-yl]-1H-indazole was consumed completely and desired mass was detected. The reaction mixture was concentrated under reduced pressure to give a residue. The residue was purified by prep-HPLC (TFA condition; column: Phenomenex Luna C18 75*30 mm*3 μm; mobile phase: [water(TFA)-ACN]; B %: 55%-85%, 8 min) to give desired 4-fluoro-3-(7-(5-fluoropyrimidin-2-yl)-4,7-diazaspiro[2.5]octan-4-yl)-1-((4-(isopropylsulfonyl)phenyl)sulfonyl)-1H-indazole (19.2 mg, 32.6 μmol, 27.9% yield, 100.00% purity) as a white solid. ¹H NMR (400 MHz, DMSO-d₆) δ 8.48 (s, 2H), 8.09-8.01 (m, 4H), 7.97 (d, J=8.4 Hz, 1H), 7.73 (br d, J=5.0 Hz, 1H), 7.26 (dd, J=8.0, 10.7 Hz, 1H), 3.90 (s, 2H), 3.65 (br s, 2H), 3.62-3.48 (m, 2H), 3.46 (s, 1H), 1.08 (d, J=6.7 Hz, 6H), 0.88 (br s, 2H), 0.51 (s, 2H). HPLC: 100.00% (220 nm), 99.72% (215 nm), 100.00% (254 nm). MS (ESI): mass calcd. For C₂₆H₂₆F₂N₆O₄S₂ 588.14 m/z found 589.2 [M+H]⁺.

### Compound 17: 4-chloro-1-((5-(isopropylsulfonyl) thiophen-2-yl) sulfonyl)-3-(4-azaspiro [2.4]heptan-4-yl)-1H-indazole

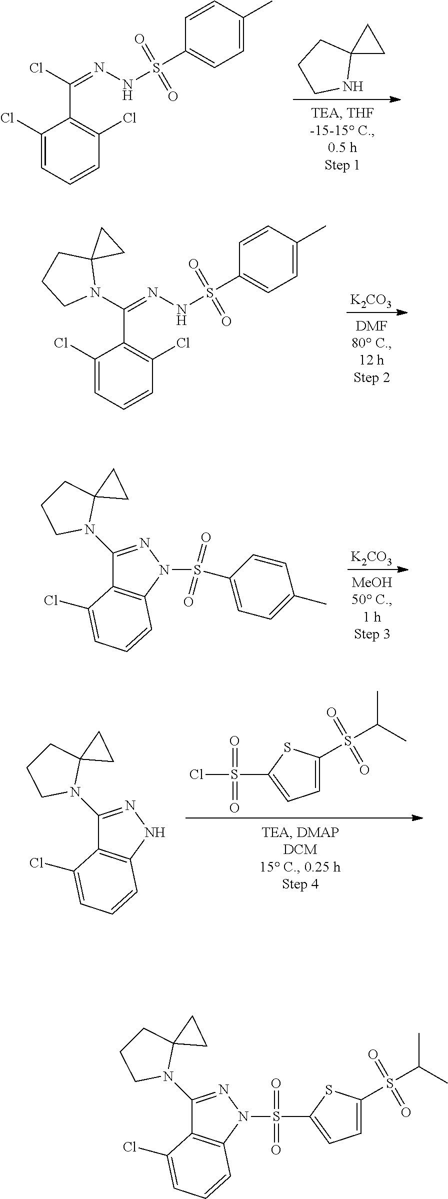

Step 1: To the solution of 4-azaspiro[2.4]heptane (0.15 g, 1.12 mmol, 1 eq, HCl) in THF (3 mL) was added TEA (1.14 g, 11.2 mmol, 1.56 mL, 10 eq) at 15° C. and the solution was stirred at 15° C. for 0.5 hour. To the reaction mixture was added (1E)-2,6-dichloro-N-(p-tolylsulfonyl)benzohydrazonoyl chloride (424 mg, 1.12 mmol, 1 eq) at −15° C. and the solution was stirred at 15° C. for 0.5 hour. LC-MS showed 4-azaspiro [2.4]heptane was consumed completely and one main peak with desired mass was detected. Then it was separated between water (20 mL) and ethyl acetate (15 mL*3). The organic phase was separated, washed with brine (20 mL), dried over Na₂SO₄, filtered and concentrated under reduced pressure to give desired (E)-N′-((2,6-dichlorophenyl)(4-azaspiro[2.4]heptan-4-yl)methylene)-4-methylbenzenesulfonohydrazide (492 mg, crude) as a brown oil. MS (ESI): mass calcd. For C₂₀H₂₁C₁₂N₃O₂S 437.07, m/z found 438.1 [M+H]⁺.

Step 2: The mixture of N—[(E)-[4-azaspiro [2.4]heptan-4-yl-(2, 6-dichlorophenyl) methylene]amino]-4-methyl-benzenesulfonamide (492 mg, 1.12 mmol, 1 eq) and K₂CO₃ (1.55 g, 11.2 mmol, 10 eq) in DMF (5 mL) was stirred at 80° C. for 12 hours. LC-MS showed N—[(E)-[4-azaspiro [2.4]heptan-4-yl-(2, 6-dichlorophenyl) methylene]amino]-4-methyl-benzenesulfonamide was consumed completely and a new spot. The reaction was concentrated to get desired 4-chloro-3-(4-azaspiro [2.4]heptan-4-yl)-1-tosyl-1H-indazole (451 mg, crude) as a brown solid. MS (ESI): mass calcd. For C₂₄H₂₂F₂N₆O₂S 401.10, m/z found 402.1 [M+H]⁺.

Step 3: The mixture of 3-(4-azaspiro [2.4]heptan-4-yl)-4-chloro-1-(p-tolylsulfonyl) indazole (451 mg, 1.12 mmol, 1 eq) and K₂CO₃ (775 mg, 5.61 mmol, 5 eq) in MeOH (5 mL) was stirred at 50° C. for 1 hour. LC-MS showed 3-(4-azaspiro [2.4]heptan-4-yl)-4-chloro-1-(p-tolylsulfonyl) indazole was consumed completely and desired mass was detected. The reaction mixture was added to water (20 mL), the aqueous phase was extracted with EtOAc (15 mL*3). The organic layer was dried over Na₂SO₄, filtered and the filtrate was concentrated to give a residue. The residue was purified by prep-TLC (SiO₂, petroleum ether/Ethyl acetate=2/1) to give desired 4-chloro-3-(4-azaspiro [2.4]heptan-4-yl)-1H-indazole (100 mg, crude) as a brown oil. MS (ESI): mass calcd. For C₁₃H₁₄ClN₃ 247.09, m/z found 248.1 [M+H]⁺.

Step 4: To a solution of 3-(4-azaspiro[2.4]heptan-4-yl)-4-chloro-1H-indazole (40 mg, 161 μmol, 1 eq) and 5-isopropylsulfonylthiophene-2-sulfonyl chloride (37.3 mg, 129 μmol, 0.8 eq) in DCM (2 mL) was added DMAP (1.97 mg, 16.2 μmol, 0.1 eq) and TEA (49.0 mg, 484 μmol, 67.4 μL, 3 eq). The mixture was stirred at 15° C. for 0.25 hour. LC-MS showed 3-(4-azaspiro [2.4]heptan-4-yl)-4-chloro-1H-indazole was consumed completely and desired mass was detected. The reaction mixture was concentrated under reduced pressure to give a residue. The residue was purified by prep-HPLC (TFA condition; column: Phenomenex C18 75*30 mm*3 m; mobile phase: [water(NH₄HCO₃)-ACN]; B %: 65%-95%, 8 min) to give desired 4-chloro-1-((5-(isopropylsulfonyl)thiophen-2-yl)sulfonyl)-3-(4-azaspiro[2.4]heptan-4-yl)-1H-indazole (3.1 mg, 6.10 μmol, 3.78% yield, 98.34% purity) as a white solid. ¹H NMR (400 MHz, DMSO-d₆) δ 7.97 (d, J=8.3 Hz, 1H), 7.80-7.77 (m, 1H), 7.77-7.74 (m, 1H), 7.64 (t, J=8.1 Hz, 1H), 7.48 (d, J=7.7 Hz, 1H), 3.63-3.54 (m, 1H), 3.46 (br t, J=6.5 Hz, 2H), 1.93-1.83 (m, 4H), 1.13 (d, J=6.8 Hz, 6H), 0.76 (s, 2H), 0.64-0.59 (m, 2H). HPLC: 90.99% (220 nm), 93.41% (215 nm), 98.34% (254 nm). MS (ESI): mass calcd. For C₂₀H₂₂ClN₃O₄S₃ 499.05 m/z found 500.1 [M+H]⁺.

### Compound 18: 4-chloro-3-(4-(5-fluoropyrimidin-2-yl)-2,2-dimethylpiperazin-1-yl)-1-((4-(isopropylsulfonyl)phenyl)sulfonyl)-1H-indazole

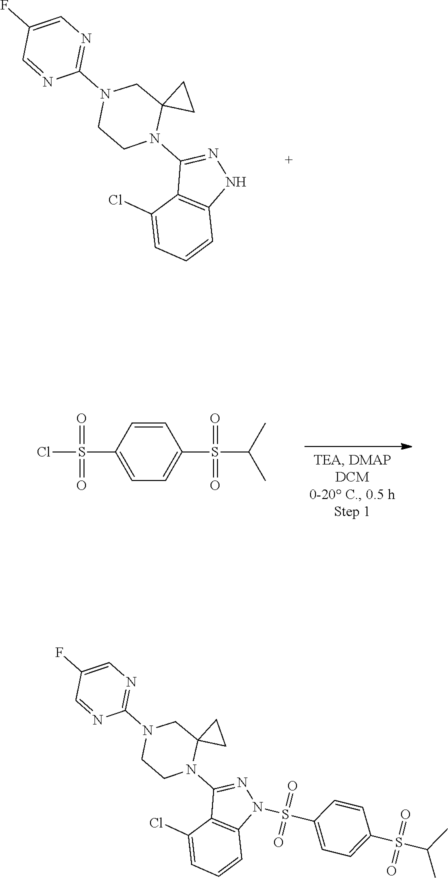 [SMILES: Fc1cnc(N2CCN(c3n[nH]c4cccc(Cl)c34)C3(CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cn4)CC34CC4)c3c(Cl)cccc32)cc1]

Step 1: To a solution of 4-chloro-3-[4-(5-fluoropyrimidin-2-yl)-2, 2-dimethyl-piperazin-1-yl]-1H-indazole (50 mg, 139 μmol, 1 eq) and 4-isopropylsulfonylbenzenesulfonyl chloride (58.8 mg, 208 μmol, 1.5 eq) in DCM (2 mL) was added TEA (28.0 mg, 277 μmol, 38.6 μL, 2 eq) and DMAP (1.69 mg, 13.9 μmol, 0.1 eq) at 0° C. The mixture was stirred at 20° C. for 0.5 hour. LC-MS showed 4-chloro-3-[4-(5-fluoropyrimidin-2-yl)-2, 2-dimethyl-piperazin-1-yl]-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge Prep OBD C18 150*40 mm*10 μm; mobile phase: [water(NH₄HCO₃)-ACN]; B %: 50%-90%, 8 min) to give desired 4-chloro-3-[4-(5-fluoropyrimidin-2-yl)-2, 2-dimethyl-piperazin-1-yl]-1-(4-isopropylsulfonylphenyl) sulfonyl-indazole (10.4 mg, 16.6 μmol, 12.0% yield, 97.14% purity) as a pale yellow solid. ¹H NMR (400 MHz, DMSO-d₆) δ 8.45 (br s, 2H), 8.20-8.01 (m, 5H), 7.68 (br t, J=7.5 Hz, 1H), 7.51 (br d, J=7.1 Hz, 1H), 3.90 (br s, 2H), 3.71-3.43 (m, 3H), 3.20 (br s, 2H), 1.21-1.02 (m, 12H). HPLC: 97.14% (220 nm), 97.00% (215 nm), 98.56% (254 nm). MS (ESI): mass calcd. For C₂₆H₂₈ClFN₆O₄S₂ 606.13, m/z found 607.2 [M+H]⁺.

### Compound 19: 4, 6-difluoro-3-[7-(5-fluoro-2-pyridyl)-4, 7-diazaspiro [2.5]octan-4-yl]-1-(4-isopropylsulfonylphenyl) sulfonyl-indazole

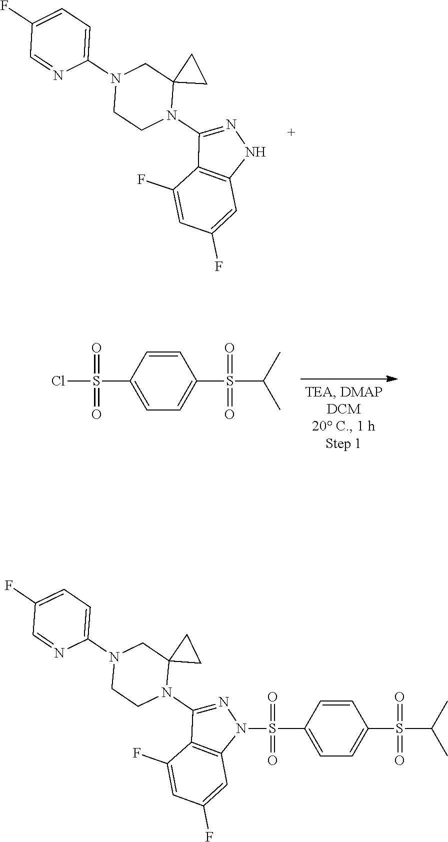 [SMILES: Fc1ccc(N2CCN(c3n[nH]c4cc(F)cc(F)c34)C3(CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ccc(F)cn4)CC34CC4)c3c(F)cc(F)cc32)cc1]

Step 1: To a solution of 4, 6-difluoro-3-[7-(5-fluoro-2-pyridyl)-4, 7-diazaspiro [2.5]octan-4-yl]-1H-indazole (20 mg, 55.7 μmol, 1 eq) and 4-isopropylsulfonylbenzenesulfonyl chloride (18.9 mg, 66.8 μmol, 1.2 eq) in DCM (1 mL) was added TEA (11.3 mg, 111 μmol, 15.5 μL, 2 eq) and DMAP (680 μg, 5.57 μmol, 0.1 eq). The mixture was stirred at 20° C. for 1 hour. LC-MS showed 4, 6-difluoro-3-[7-(5-fluoro-2-pyridyl)-4, 7-diazaspiro [2.5]octan-4-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-HPLC (FA condition; Method: column: Phenomenex Luna 80*30 mm*3 μm; mobile phase: [water(FA)-ACN]; B %: 45%-80%, 8 min) to give desired 4, 6-difluoro-3-[7-(5-fluoro-2-pyridyl)-4, 7-diazaspiro [2.5]octan-4-yl]-1-(4-isopropylsulfonylphenyl) sulfonyl-indazole (10.1 mg, 16.7 μmol, 30.0% yield, 100.00% purity) as a white solid. ¹H NMR (400 MHz, DMSO-d₆) δ 8.14-8.06 (m, 3H), 7.98 (d, J=8.4 Hz, 2H), 7.72 (dd, J=1.6, 8.8 Hz, 1H), 7.52 (dt, J=3.1, 8.8 Hz, 1H), 7.38 (s, 1H), 6.86 (dd, J=3.3, 9.3 Hz, 1H), 3.64 (br t, J=4.7 Hz, 2H), 3.57 (s, 2H), 3.39 (s, 1H), 3.30 (br s, 2H), 1.05 (d, J=6.8 Hz, 6H), 0.88-0.82 (m, 2H), 0.50 (s, 2H). HPLC: 100.00% (220 nm), 99.78% (215 nm), 100.00% (254 nm). MS (ESI): mass calcd. For C₂₇H₂₆F₃N₅O₄S₂ 605.14, m/z found 606.2 [M+H]⁺.

### Compound 20: 4, 6-difluoro-3-[7-(5-fluoropyrimidin-2-yl)-4, 7-diazaspiro [2.5]octan-4-yl]-1-(4-isopropylsulfonylphenyl) sulfonyl-indazole

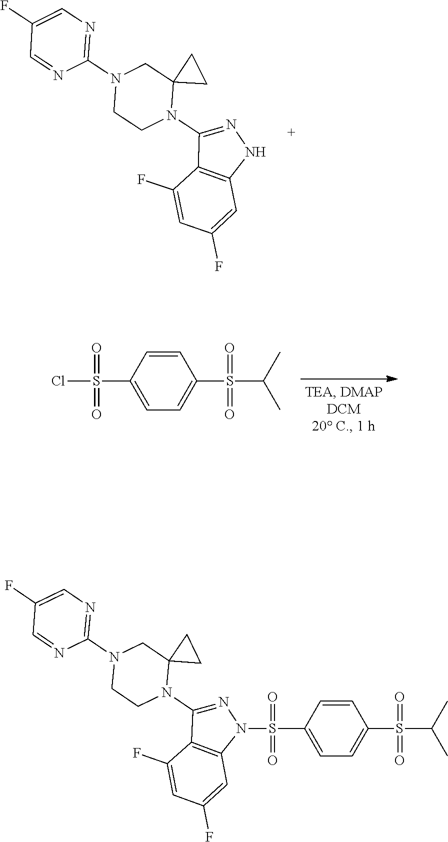 [SMILES: Fc1cnc(N2CCN(c3n[nH]c4cc(F)cc(F)c34)C3(CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cn4)CC34CC4)c3c(F)cc(F)cc32)cc1]

Step 1: To a solution of 4, 6-difluoro-3-[7-(5-fluoropyrimidin-2-yl)-4, 7-diazaspiro [2.5]octan-4-yl]-1H-indazole (20 mg, 55.5 μmol, 1 eq) and 4-isopropylsulfonylbenzenesulfonyl chloride (18.8 mg, 66.6 μmol, 1.2 eq) in DCM (1 mL) was added DMAP (678 μg, 5.55 μmol, 0.1 eq) and TEA (11.2 mg, 111 μmol, 15.5 μL, 2 eq). The mixture was stirred at 20° C. for 1 hour. LC-MS showed 4, 6-difluoro-3-[7-(5-fluoropyrimidin-2-yl)-4, 7-diazaspiro [2.5]octan-4-yl]-1H-indazole was consumed completely and desired mass was detected. The reaction mixture was partitioned between 10 mL of H₂O and 10 mL of EtOAc. The organic phase was separated, washed with 10 mL of brine, dried over Na₂SO₄, filtered and concentrated under reduced pressure to give the crude product. The residue was purified by prep-HPLC (FA condition; Method: column: Phenomenex Luna C18 75*30 mm*3 μm; mobile phase: [water(FA)-ACN]; B %: 65%-95%, 8 min) to give desired 4, 6-difluoro-3-[7-(5-fluoropyrimidin-2-yl)-4, 7-diazaspiro [2.5]octan-4-yl]-1-(4-isopropylsulfonylphenyl)sulfonyl-indazole (17.3 mg, 28.2 μmol, 50.8% yield, 98.80% purity) as a white solid. ¹H NMR (400 MHz, DMSO-d₆) δ 8.45 (s, 2H), 8.13-8.08 (m, 2H), 8.04-8.00 (m, 2H), 7.72 (dd, J=1.9, 8.8 Hz, 1H), 7.42-7.34 (m, 1H), 3.85 (s, 2H), 3.60 (br d, J=2.4 Hz, 2H), 3.44 (br d, J=6.8 Hz, 1H), 3.30 (s, 2H), 1.07 (d, J=6.9 Hz, 6H), 0.84 (s, 2H), 0.48 (s, 2H). HPLC: 98.80% (220 nm), 98.82% (215 nm), 98.98% (254 nm). MS (ESI): mass calcd. For C₂₆H₂₅F₃N₆O₄S₂ 606.13, m/z found 607.1 [M+H]⁺.

### Compound 21: 4-chloro-6-fluoro-3-[7-(5-fluoropyrimidin-2-yl)-4, 7-diazaspiro [2.5]octan-4-yl]-1-(4-isopropylsulfonylphenyl) sulfonyl-indazole

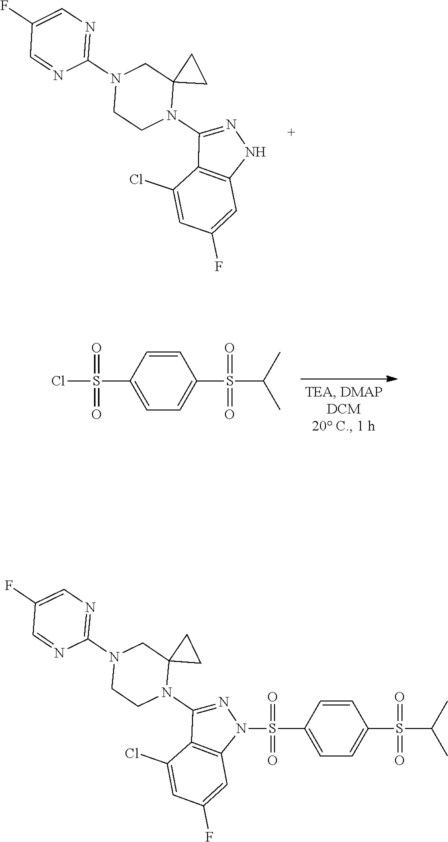 [SMILES: Fc1cnc(N2CCN(c3n[nH]c4cc(F)cc(Cl)c34)C3(CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cn4)CC34CC4)c3c(Cl)cc(F)cc32)cc1]

Step 1: To a solution of 4-chloro-6-fluoro-3-[7-(5-fluoropyrimidin-2-yl)-4, 7-diazaspiro [2.5]octan-4-yl]-1H-indazole (20.9 mg, 55.5 μmol, 1 eq) and 4-isopropylsulfonylbenzenesulfonyl chloride (18.8 mg, 66.6 μmol, 1.2 eq) in DCM (1 mL) was added TEA (11.2 mg, 111 μmol, 15.5 μL, 2 eq) and DMAP (678 μg, 5.55 μmol, 0.1 eq). The mixture was stirred at 20° C. for 1 hour. LC-MS showed 4-chloro-6-fluoro-3-[7-(5-fluoropyrimidin-2-yl)-4, 7-diazaspiro [2.5]octan-4-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-HPLC (FA condition; Method:column: Phenomenex Luna C18 75*30 mm*3 um; mobile phase: [water(FA)-ACN]; B %: 65%-95%, 8 min) to give desired 4-chloro-6-fluoro-3-[7-(5-fluoropyrimidin-2-yl)-4, 7-diazaspiro [2.5]octan-4-yl]-1-(4-isopropylsulfonylphenyl) sulfonyl-indazole (23.2 mg, 37.0 μmol, 66.6% yield, 99.29% purity) as a white solid. ¹H NMR (400 MHz, DMSO-d₆) δ 8.45 (s, 2H), 8.15-8.08 (m, 2H), 8.06-8.00 (m, 2H), 7.86-7.81 (m, 1H), 7.56 (dd, J=2.1, 9.1 Hz, 1H), 3.89 (s, 2H), 3.59 (br s, 2H), 3.54-3.49 (m, 2H), 3.49-3.42 (m, 1H), 1.08 (d, J=6.9 Hz, 6H), 0.75 (s, 2H), 0.34 (br s, 2H). HPLC: 99.29% (220 nm), 99.44% (215 nm), 99.90% (254 nm). MS (ESI): mass calcd. For C₂₆H₂₅ClF₂N₆O₄S₂ 622.10, m/z found 623.1 [M+H]⁺.

### Compound 22: 4-chloro-1-((4-((2-fluoropropan-2-yl) sulfonyl) phenyl) sulfonyl)-3-(7-(5-fluoropyrimidin-2-yl)-4, 7-diazaspiro [2.5]octan-4-yl)-1H-indazole

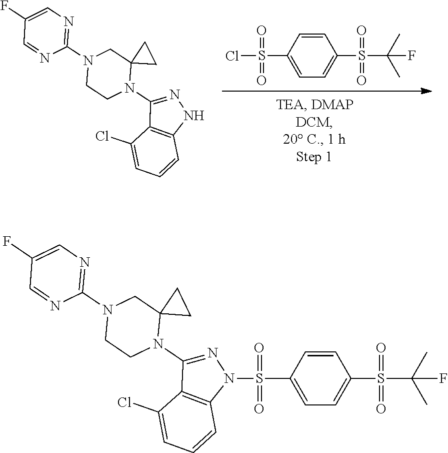 [SMILES: Fc1cnc(N2CCN(c3n[nH]c4cccc(Cl)c34)C3(CC3)C2)nc1 ; CC(C)(F)S(=O)(=O)c1ccc(S(=O)(=O)Cl)cc1 ; CC(C)(F)S(=O)(=O)c1ccc(S(=O)(=O)n2nc(N3CCN(c4ncc(F)cn4)CC34CC4)c3c(Cl)cccc32)cc1]

Step 1: To the solution of 4-chloro-3-[7-(5-fluoropyrimidin-2-yl)-4,7-diazaspiro[2.5]octan-4-yl]-1H-indazole (30 mg, 83.6 μmol, 1 eq), TEA (42.3 mg, 418 μmol, 5 eq) and DMAP (1.02 mg, 8.36 μmol, 0.1 eq) in DCM (2 mL) was added 4-(1-fluoro-1-methyl-ethyl)sulfonylbenzenesulfonyl chloride (25.2 mg, 83.6 μmol, 1 eq) and the solution was stirred at 20° C. for 1 hour. LC-MS showed 4-chloro-3-[7-(5-fluoropyrimidin-2-yl)-4, 7-diazaspiro [2.5]octan-4-yl]-1H-indazole remained and desired mass was detected. The reaction was concentrated to get a residue. The residue was purified by prep-HPLC (column: Waters Xbridge Prep OBD C18 150*40 mm*10 μm; mobile phase: [water(NH4HCO3)-ACN]; B %: 45%-95%, 8 min) to give desired 4-chloro-1-[4-(1-fluoro-1-methyl-ethyl)sulfonylphenyl]sulfonyl-3-[7-(5-fluoropyrimidin-2-yl)-4,7-diazaspiro[2.5]octan-4-yl]indazole (20 mg, 30.3 μmol, 36.17% yield, 94.23% purity) as a white solid. ¹H NMR (400 MHz, DMSO-d₆) δ 8.45 (s, 2H), 8.01-8.15 (m, 4H), 7.66 (br m, 1H), 7.49 (br d, 1H), 3.91 (br s, 2H), 3.47-3.69 (m, 5H), 1.46-1.72 (m, 6H), 0.75 (br s, 2H), 0.33 (br s, 2H). HPLC: 94.23% (220 nm), 94.03% (215 nm), 99.09% (254 nm). MS (ESI): mass calcd. For C₂₆H₂₅N₆S₂O₄ClF₂ 622.1, m/z found 623.0 [M+H]⁺.

### Compound 23: 4-chloro-3-[7-(5-fluoro-2-pyridyl)-4, 7-diazaspiro [2.5]octan-4-yl]-1-(4-isopropylsulfonylphenyl) sulfonyl-indazole

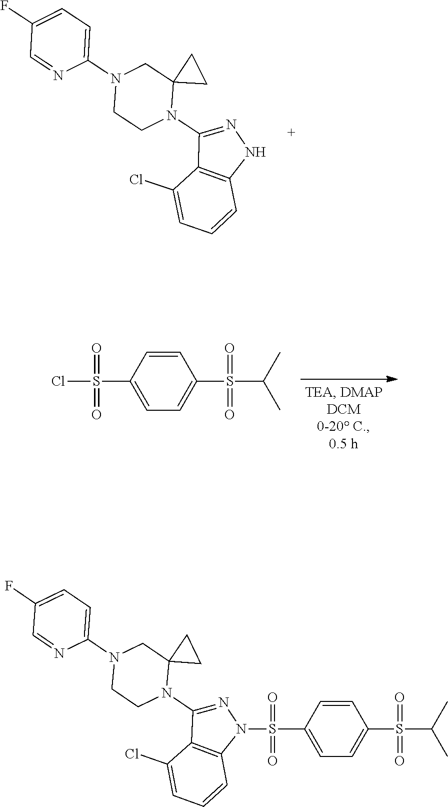 [SMILES: Fc1ccc(N2CCN(c3n[nH]c4cccc(Cl)c34)C3(CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ccc(F)cn4)CC34CC4)c3c(Cl)cccc32)cc1]

Step 1: To a solution of 4-isopropylsulfonylbenzenesulfonyl chloride (59.3 mg, 210 mol, 1.5 eq) and 4-chloro-3-[7-(5-fluoro-2-pyridyl)-4,7-diazaspiro[2.5]octan-4-yl]-1H-indazole (50 mg, 140 μmol, 1 eq) in DCM (2 mL) was added TEA (28.3 mg, 279 μmol, 38.9 μL, 2 eq) and DMAP (1.71 mg, 14.0 μmol, 0.1 eq) at 0° C. The mixture was stirred at 20° C. for 0.5 hour. LC-MS showed 4-chloro-3-[7-(5-fluoro-2-pyridyl)-4, 7-diazaspiro [2.5]octan-4-yl]-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge BEH C18 100*30 mm*10 um; mobile phase: [water(NH₄HCO₃)-ACN]; B %: 65%-95%, 6 min) to give desired 4-chloro-3-[7-(5-fluoro-2-pyridyl)-4, 7-diazaspiro [2.5]octan-4-yl]-1-(4-isopropylsulfonylphenyl)sulfonyl-indazole (15.7 mg, 25.5 μmol, 18.3% yield, 98.11% purity) as a pale yellow solid. ¹H NMR (400 MHz, DMSO-d₆) δ 8.14-8.02 (m, 4H), 8.01-7.95 (m, 2H), 7.66 (t, J=8.1 Hz, 1H), 7.58-7.45 (m, 2H), 6.94-6.84 (m, 1H), 3.65 (br s, 2H), 3.58 (br s, 2H), 3.48-3.36 (m, 2H), 3.33 (br s, 1H), 1.07 (d, J=6.9 Hz, 6H), 0.78 (br s, 2H), 0.37 (br s, 2H). HPLC: 98.11% (220 nm), 98.13% (215 nm), 98.19% (254 nm). MS (ESI): mass calcd. For C₂₇H₂₇ClFN₅O₄S₂ 603.12, m/z found 603.9 [M+H]⁺.

### Compound 24: 4-chloro-3-[7-(3, 5-difluoro-2-pyridyl)-4, 7-diazaspiro [2.5]octan-4-yl]-1-(4-isopropylsulfonylphenyl) sulfonyl-indazole

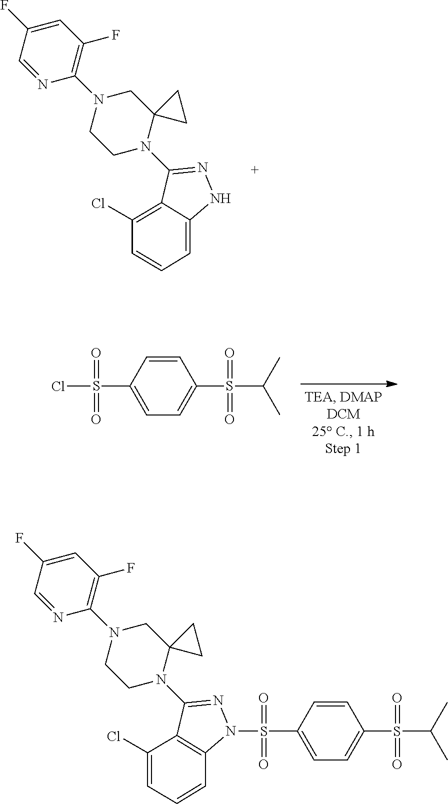 [SMILES: Fc1cnc(N2CCN(c3n[nH]c4cccc(Cl)c34)C3(CC3)C2)c(F)c1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cc4F)CC34CC4)c3c(Cl)cccc32)cc1]

Step 1: To a solution of 4-chloro-3-[7-(3, 5-difluoro-2-pyridyl)-4, 7-diazaspiro [2.5]octan-4-yl]-1H-indazole (30 mg, 79.8 μmol, 1 eq) and 4-isopropylsulfonylbenzenesulfonyl chloride (33.9 mg, 120 μmol, 1.5 eq) in DCM (1 mL) was added TEA (16.2 mg, 160 μmol, 22.2 μL, 2 eq) and DMAP (975 μg, 7.98 μmol, 0.1 eq). The mixture was stirred at 25° C. for 1 hour. LC-MS showed 4-chloro-3-[7-(3, 5-difluoro-2-pyridyl)-4, 7-diazaspiro [2.5]octan-4-yl]-1H-indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-HPLC (FA condition; according to LCMS; Method: column: Phenomenex C18 75*30 mm*3 m; mobile phase: [water(FA)-ACN]; B %: 70%-90%, 8 min) to give desired 4-chloro-3-[7-(3,5-difluoro-2-pyridyl)-4,7-diazaspiro[2.5]octan-4-yl]-1-(4-isopropylsulfonylphenyl)sulfonyl-indazole (21.9 mg, 35.0 μmol, 43.81% yield, 99.34% purity) as a little yellow solid. ¹H NMR (400 MHz, DMSO-d₆) δ 8.23-7.92 (m, 6H), 7.91-7.79 (m, 1H), 7.76-7.61 (m, 1H), 7.57-7.44 (m, 1H), 3.61 (br s, 2H), 3.30 (br s, 3H), 2.48-2.40 (m, 2H), 1.02 (br d, J=6.4 Hz, 6H), 0.90-0.68 (m, 2H), 0.53-0.28 (m, 2H). HPLC: 99.34% (220 nm), 99.07% (215 nm), 99.89% (254 nm). MS (ESI): mass calcd. For C₂₇H₂₆ClF₂N₅O₄S₂ 621.1, m/z found 622.1 [M+H]⁺.

### Compound 25: 4-fluoro-3-[7-(5-fluoro-2-pyridyl)-4, 7-diazaspiro [2.5]octan-4-yl]-1-(4-isopropylsulfonylphenyl) sulfonyl-indazole

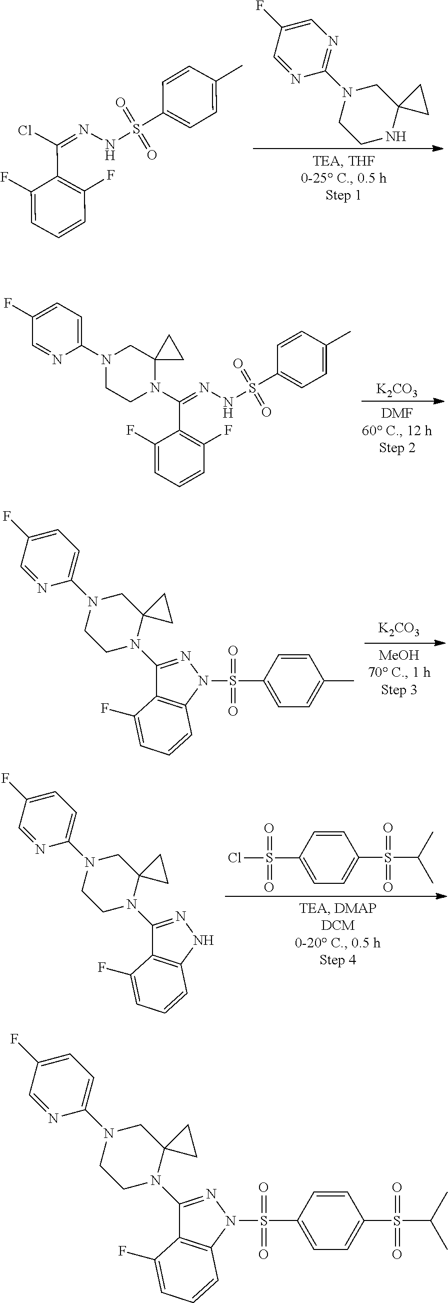

Step 1: To a solution of 7-(5-fluoro-2-pyridyl)-4,7-diazaspiro[2.5]octane (250 mg, 1.21 mmol, 1 eq) in THF (3 mL) was added TEA (1.22 g, 12.1 mmol, 1.68 mL, 10 eq) dropwise at 25° C. After addition, the mixture was stirred at this temperature for 10 minutes, and then (1Z)-2, 6-difluoro-N-(p-tolylsulfonyl) benzohydrazonoyl chloride (459 mg, 1.33 mmol, 1.1 eq) in THF (3 mL) was added dropwise at 0° C. The resulting mixture was stirred at 25° C. for 20 minutes. LC-MS showed 7-(5-fluoro-2-pyridyl)-4, 7-diazaspiro [2.5]octane was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(Z)-[(2,6-difluorophenyl)-[7-(5-fluoro-2-pyridyl)-4, 7-diazaspiro [2.5]octan-4-yl]methylene]amino]-4-methyl-benzenesulfonamide (750 mg, crude) as a yellow solid. MS (ESI): mass calcd. For C₂₅H₂₄F₃N₅O₂S 515.15, m/z found 516.1 [M+H]⁺.

Step 2: To a solution of N—[(Z)-[(2, 6-difluorophenyl)-[7-(5-fluoro-2-pyridyl)-4, 7-diazaspiro [2.5]octan-4-yl]methylene]amino]-4-methyl-benzenesulfonamide (750 mg, 1.45 mmol, 1 eq) in DMF (10 mL) was added K₂CO₃ (804 mg, 5.82 mmol, 4 eq). The mixture was stirred at 60° C. for 12 hours. LC-MS showed N—[(Z)-[(2, 6-difluorophenyl)-[7-(5-fluoro-2-pyridyl)-4, 7-diazaspiro [2.5]octan-4-yl]methylene]amino]-4-methyl-benzenesulfonamide was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give desired 4-fluoro-3-[7-(5-fluoro-2-pyridyl)-4,7-diazaspiro[2.5]octan-4-yl]-1-(p-tolylsulfonyl)indazole (720 mg, crude) as an orange oil. MS (ESI): mass calcd. For C₂₅H₂₃F₂N₅O₂S 495.15, m/z found 496.0 [M+H]⁺.

Step 3: To a solution of 4-fluoro-3-[7-(5-fluoro-2-pyridyl)-4, 7-diazaspiro [2.5]octan-4-yl]-1-(p-tolylsulfonyl) indazole (720 mg, 1.45 mmol, 1 eq) in MeOH (10 mL) was added K₂CO₃ (1.00 g, 7.26 mmol, 5 eq). The mixture was stirred at 70° C. for 1 hour. LC-MS showed 4-fluoro-3-[7-(5-fluoro-2-pyridyl)-4, 7-diazaspiro [2.5]octan-4-yl]-1-(p-tolylsulfonyl) indazole was consumed completely and desired compound was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-TLC (SiO₂, petroleum ether/Ethyl acetate=3/1) to give desired 4-fluoro-3-[7-(5-fluoro-2-pyridyl)-4, 7-diazaspiro [2.5]octan-4-yl]-1H-indazole (180 mg, crude) as a yellow oil. MS (ESI): mass calcd. For C₁₈H₁₇F₂N₅ 341.15, m/z found 342.1 [M+H]⁺.

Step 4: To a solution of 4-isopropylsulfonylbenzenesulfonyl chloride (62.1 mg, 220 mol, 1.5 eq) and 4-fluoro-3-[7-(5-fluoro-2-pyridyl)-4,7-diazaspiro[2.5]octan-4-yl]-1H-indazole (50 mg, 146 μmol, 1 eq) in DCM (2 mL) was added TEA (29.6 mg, 293 μmol, 40.8 μL, 2 eq) and DMAP (1.79 mg, 14.7 μmol, 0.1 eq) at 0° C. The mixture was stirred at 20° C. for 0.5 hour. LC-MS showed 4-fluoro-3-[7-(5-fluoro-2-pyridyl)-4, 7-diazaspiro [2.5]octan-4-yl]-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water(NH₄HCO₃)-ACN]; B %: 60%-90%, 6 min) to give desired 4-fluoro-3-[7-(5-fluoro-2-pyridyl)-4,7-diazaspiro[2.5]octan-4-yl]-1-(4-isopropylsulfonylphenyl)sulfonyl-indazole (3.0 mg, 5.04 μmol, 3.44% yield, 98.64% purity) as a white solid. ¹H NMR (400 MHz, DMSO-d₆) δ 8.15 (d, J=3.0 Hz, 1H), 8.12-8.07 (m, 2H), 8.05-7.98 (m, 3H), 7.77 (dt, J=5.1, 8.2 Hz, 1H), 7.59 (dt, J=3.1, 8.8 Hz, 1H), 7.30 (dd, J=8.1, 10.7 Hz, 1H), 6.93 (dd, J=3.3, 9.3 Hz, 1H), 3.73 (br t, J=4.7 Hz, 2H), 3.66 (s, 2H), 3.48-3.43 (m, 1H), 3.37 (s, 2H), 1.10 (d, J=6.8 Hz, 6H), 0.93 (s, 2H), 0.58 (s, 2H). HPLC: 98.64% (220 nm), 98.45% (215 nm), 97.69% (254 nm). MS (ESI): mass calcd. For C₂₇H₂₇F₂N₅O₄S₂ 587.15, m/z found 588.1 [M+H]⁺.

### Compound 26: 3-[7-(3, 5-difluoro-2-pyridyl)-4, 7-diazaspiro [2.5]octan-4-yl]-4-fluoro-1-(4-isopropylsulfonylphenyl) sulfonyl-indazole

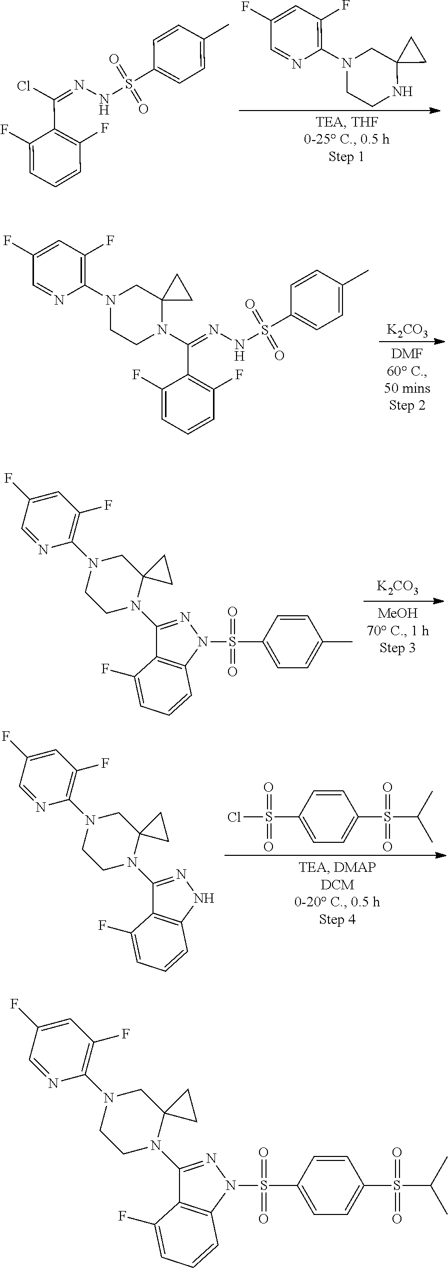

Step 1: To a solution of 7-(3, 5-difluoro-2-pyridyl)-4, 7-diazaspiro [2.5]octane (250 mg, 1.11 mmol, 1 eq) in THF (3 mL) was added dropwise TEA (1.12 g, 11.1 mmol, 1.54 mL, 10 eq) at 25° C. After addition, the mixture was stirred at this temperature for 10 minutes, and then (1Z)-2,6-difluoro-N-(p-tolylsulfonyl) benzohydrazonoyl chloride (421 mg, 1.22 mmol, 1.1 eq) in THF (3 mL) was added dropwise at 0° C. The resulting mixture was stirred at 25° C. for 20 minutes. LC-MS showed 7-(3, 5-difluoro-2-pyridyl)-4, 7-diazaspiro [2.5]octane was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(Z)-[(2, 6-difluorophenyl)-[7-(3, 5-difluoro-2-pyridyl)-4, 7-diazaspiro [2.5]octan-4-yl]methylene]amino]-4-methyl-benzenesulfonamide (600 mg, crude) as a yellow solid. MS (ESI): mass calcd. For C₂₅H₂₃F₄N₅O₂S 533.15, m/z found 534.1 [M+H]⁺.

Step 2: To a solution of N—[(Z)-[(2, 6-difluorophenyl)-[7-(3, 5-difluoro-2-pyridyl)-4, 7-diazaspiro [2.5]octan-4-yl]methylene]amino]-4-methyl-benzenesulfonamide (600 mg, 1.12 mmol, 1 eq) in DMF (10 mL) was added K₂CO₃ (622 mg, 4.50 mmol, 4 eq). The mixture was stirred at 60° C. for 50 minutes. LC-MS showed was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give desired 3-[7-(3, 5-difluoro-2-pyridyl)-4, 7-diazaspiro [2.5]octan-4-yl]-4-fluoro-1-(p-tolylsulfonyl)indazole (600 mg, crude) as an orange oil. MS (ESI): mass calcd. For C₂₅H₂₂F₃N₅O₂S 513.14, m/z found 514.0 [M+H]⁺.

Step 3: To a solution of 3-[7-(3, 5-difluoro-2-pyridyl)-4, 7-diazaspiro [2.5]octan-4-yl]-4-fluoro-1-(p-tolylsulfonyl) indazole (600 mg, 1.17 mmol, 1 eq) in MeOH (10 mL) was added K₂CO₃ (807 mg, 5.84 mmol, 5 eq). The mixture was stirred at 70° C. for 1 hour. LC-MS showed 3-[7-(3, 5-difluoro-2-pyridyl)-4, 7-diazaspiro [2.5]octan-4-yl]-4-fluoro-1-(p-tolylsulfonyl) indazole was consumed completely and desired compound was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-TLC (SiO₂, petroleum ether/Ethyl acetate=3/1) to give desired 3-[7-(3, 5-difluoro-2-pyridyl)-4, 7-diazaspiro [2.5]octan-4-yl]-4-fluoro-1H-indazole (200 mg, crude) as a yellow oil. MS (ESI): mass calcd. For C₁₈H₁₆F₃N₅ 359.14, m/z found 360.1 [M+H]⁺.

Step 4: To a solution of 4-isopropylsulfonylbenzenesulfonyl chloride (59.0 mg, 209 μmol, 1.5 eq) and 3-[7-(3, 5-difluoro-2-pyridyl)-4, 7-diazaspiro [2.5]octan-4-yl]-4-fluoro-1H-indazole (50 mg, 139 μmol, 1 eq) in DCM (2 mL) was added TEA (28.2 mg, 278 μmol, 38.7 uL, 2 eq) and DMAP (1.70 mg, 13.9 μmol, 0.1 eq) at 0° C. The mixture was stirred at 20° C. for 0.5 hour. LC-MS showed 3-[7-(3, 5-difluoro-2-pyridyl)-4, 7-diazaspiro [2.5]octan-4-yl]-4-fluoro-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water(NH₄HCO₃)-ACN]; B %: 65%-95%, 6 min) to give desired 3-[7-(3,5-difluoro-2-pyridyl)-4,7-diazaspiro[2.5]octan-4-yl]-4-fluoro-1-(4-isopropylsulfonylphenyl)sulfonyl-indazole (23.2 mg, 38.2 μmol, 27.5% yield, 99.70% purity) as a white solid. ¹H NMR (400 MHz, DMSO-d₆) δ 8.12 (br s, 1H), 8.02 (br d, J=13.5 Hz, 5H), 7.82 (br s, 1H), 7.72 (br s, 1H), 7.24 (br s, 1H), 3.72 (br s, 2H), 3.49 (br s, 2H), 3.44-3.40 (m, 1H), 3.21-3.00 (m, 2H), 1.06 (br d, J=4.0 Hz, 6H), 0.87 (br s, 2H), 0.54 (br s, 2H). HPLC: 99.70% (220 nm), 99.67% (215 nm), 100.00% (254 nm). MS (ESI): mass calcd. For C₂₇H₂₆F₃N₅O₄S₂ 605.14, m/z found 606.0 [M+H]⁺.

### Compound 27: 4-chloro-3-[7-(3, 5-difluoro-2-pyridyl)-4, 7-diazaspiro [2.5]octan-4-yl]-6-fluoro-1-(4-isopropylsulfonylphenyl)sulfonyl-indazole

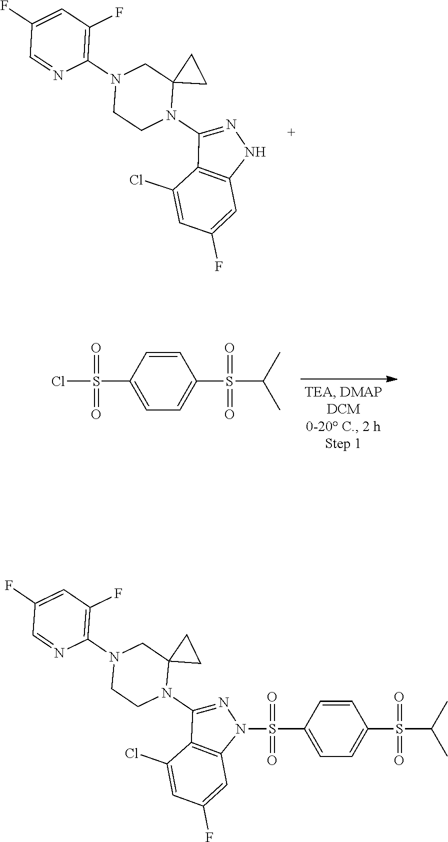 [SMILES: Fc1cnc(N2CCN(c3n[nH]c4cc(F)cc(Cl)c34)C3(CC3)C2)c(F)c1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cc4F)CC34CC4)c3c(Cl)cc(F)cc32)cc1]

Step 1: To a solution of 4-chloro-3-[7-(3, 5-difluoro-2-pyridyl)-4, 7-diazaspiro [2.5]octan-4-yl]-6-fluoro-1H-indazole (50 mg, 127 μmol, 1 eq) and 4-isopropylsulfonylbenzenesulfonyl chloride (53.9 mg, 190 μmol, 1.5 eq) in DCM (2 mL) was added TEA (25.7 mg, 254 μmol, 35.4 μL, 2 eq) and DMAP (1.55 mg, 12.7 μmol, 0.1 eq) at 0° C. The mixture was stirred at 20° C. for 2 hours. LC-MS showed 4-chloro-3-[7-(3, 5-difluoro-2-pyridyl)-4, 7-diazaspiro [2.5]octan-4-yl]-6-fluoro-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; Waters Xbridge Prep OBD C18 150*40 mm*10 μm; mobile phase: [water(NH₄HCO₃)-ACN]; B %: 55%-90%, 8 min) to give desired 4-chloro-3-[7-(3, 5-difluoro-2-pyridyl)-4, 7-diazaspiro [2.5]octan-4-yl]-6-fluoro-1-(4-isopropylsulfonylphenyl) sulfonyl-indazole (28.5 mg, 42.3 μmol, 33.3% yield, 98.311% purity) as a yellow solid. ¹H NMR (400 MHz, DMSO-d₆) δ 8.15-8.08 (m, 3H), 8.00 (d, J=8.4 Hz, 2H), 7.87-7.77 (m, 2H), 7.57 (dd, J=1.7, 9.0 Hz, 1H), 3.59 (br s, 2H), 3.45 (s, 2H), 3.32 (br s, 1H), 3.25-3.04 (m, 2H), 1.07 (d, J=6.7 Hz, 6H), 0.75 (br s, 2H), 0.37 (br s, 2H). HPLC: 98.31% (220 nm), 98.12% (215 nm), 98.97% (254 nm). MS (ESI): mass calcd. For C₂₇H₂₅N₅ClF₃S₂O₄ 639.10 m/z found 640.1 [M+H]⁺.

### Compound 28: 3-[7-(3,5-difluoro-2-pyridyl)-4,7-diazaspiro[2.5]octan-4-yl]-4,6-difluoro-1-(4-isopropylsulfonylphenyl)sulfonyl-indazole

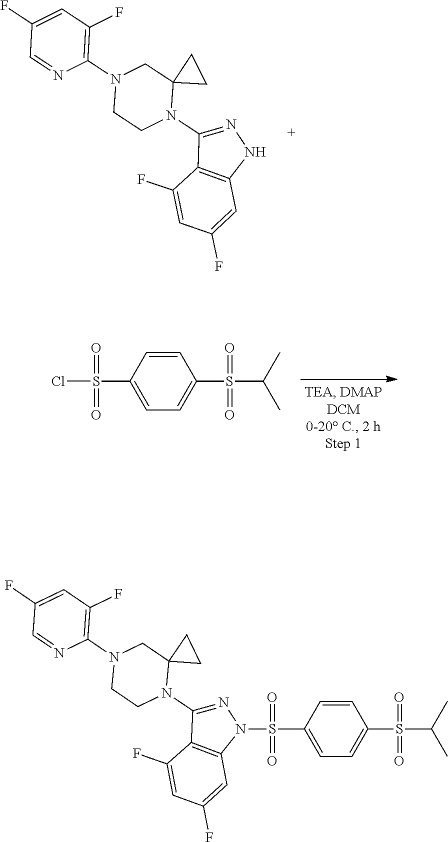 [SMILES: Fc1cnc(N2CCN(c3n[nH]c4cc(F)cc(F)c34)C3(CC3)C2)c(F)c1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cc4F)CC34CC4)c3c(F)cc(F)cc32)cc1]

Step 1: To a solution of 3-[7-(3, 5-difluoro-2-pyridyl)-4, 7-diazaspiro [2.5]octan-4-yl]-4, 6-difluoro-1H-indazole (27 mg, 71.6 μmol, 1 eq) and 4-isopropylsulfonylbenzenesulfonyl chloride (30.4 mg, 107 μmol, 1.5 eq) in DCM (2 mL) was added TEA (14.5 mg, 143 μmol, 19.9 μL, 2 eq) and DMAP (874 μg, 7.16 μmol, 0.1 eq) at 0° C. The mixture was stirred at 20° C. for 2 hours. LC-MS showed 3-[7-(3, 5-difluoro-2-pyridyl)-4, 7-diazaspiro [2.5]octan-4-yl]-4, 6-difluoro-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge Prep OBD C18 150*40 mm*10 um; mobile phase: [water(NH₄HCO₃)-ACN]; B %: 50%-85%, 8 min) to give desired 3-[7-(3, 5-difluoro-2-pyridyl)-4, 7-diazaspiro [2.5]octan-4-yl]-4, 6-difluoro-1-(4-isopropylsulfonylphenyl) sulfonyl-indazole (8.7 mg, 13.3 μmol, 18.5% yield, 96.14% purity) as a pale yellow solid. ¹H NMR (400 MHz, DMSO-d₆) δ 8.12-8.07 (m, 3H), 7.99 (d, J=8.5 Hz, 2H), 7.85-7.76 (m, 1H), 7.72 (dd, J=1.8, 8.8 Hz, 1H), 7.43-7.33 (m, 1H), 3.68 (br t, J=4.7 Hz, 2H), 3.52-3.36 (m, 5H), 1.06 (d, J=6.8 Hz, 6H), 0.84 (br s, 2H), 0.50 (br s, 2H). HPLC: 96.14% (220 nm), 96.46% (215 nm), 96.71% (254 nm). MS (ESI): mass calcd. For C₂₇H₂₅N₅F₄S₂O₄ 623.13 m/z found 624.1 [M+H]⁺.

### Compound 29: 4-chloro-1-((4-(cyclopropylsulfonyl) phenyl) sulfonyl)-3-(7-(5-fluoropyrimidin-2-yl)-4, 7-diazaspiro [2.5]octan-4-yl)-1H-indazole

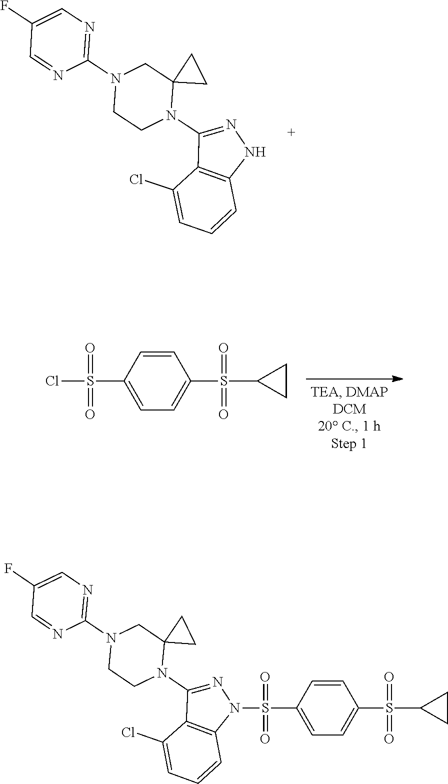 [SMILES: Fc1cnc(N2CCN(c3n[nH]c4cccc(Cl)c34)C3(CC3)C2)nc1 ; O=S(=O)(Cl)c1ccc(S(=O)(=O)C2CC2)cc1 ; O=S(=O)(c1ccc(S(=O)(=O)n2nc(N3CCN(c4ncc(F)cn4)CC34CC4)c3c(Cl)cccc32)cc1)C1CC1]

Step 1: The solution of 4-chloro-3-[7-(5-fluoropyrimidin-2-yl)-4,7-diazaspiro[2.5]octan-4-yl]-1H-indazole (25 mg, 69.7 μmol, 1 eq), TEA (35.3 mg, 348 umol, 5 eq) and DMAP (851 μg, 6.97 μmol, 0.1 eq) in DCM (2 mL) was added 4-cyclopropylsulfonylbenzenesulfonyl chloride (25.4 mg, 90.6 μmol, 1.3 eq) and the solution was stirred at 20° C. for 1 hour. LC-MS showed 4-chloro-3-[7-(5-fluoropyrimidin-2-yl)-4, 7-diazaspiro [2.5]octan-4-yl]-1H-indazole remained and desired mass was detected. The reaction was concentrated to get a residue. The residue was purified by prep-HPLC (column: Waters Xbridge Prep OBD C18 150*40 mm*10 μm; mobile phase: [water(NH₄HCO₃)-ACN]; B %: 50%-80%, 8 min) to give desired 4-chloro-1-(4-cyclopropylsulfonylphenyl)sulfonyl-3-[7-(5-fluoropyrimidin-2-yl)-4,7-diazaspiro[2.5]octan-4-yl]indazole (7.7 mg, 12.3 μmol, 17.7% yield, 96.3% purity) as a white solid. ¹H NMR (400 MHz, DMSO-d₆) δ 8.46 (s, 2H), 7.97-8.14 (m, 4H), 7.66 (m, 1H), 7.48 (d, 1H), 3.93 (s, 2H), 3.49-3.69 (m, 5H), 2.85-2.92 (m, 1H), 0.99-1.15 (m, 4H), 0.77 (br s, 2H), 0.35 (br s, 2H). HPLC: 96.33% (220 nm), 94.30% (215 nm), 100.0% (254 nm). MS (ESI): mass calcd. For C₂₆H₂₄N₆S₂O₄ClF 602.1 m/z found 603.0 [M+H]⁺.

### Compound 30: 4-chloro-3-[7-(5-chloro-3-fluoro-2-pyridyl)-4, 7-diazaspiro [2.5]octan-4-yl]-1-[(5-isopropylsulfonyl-2-thienyl) sulfonyl]indazole

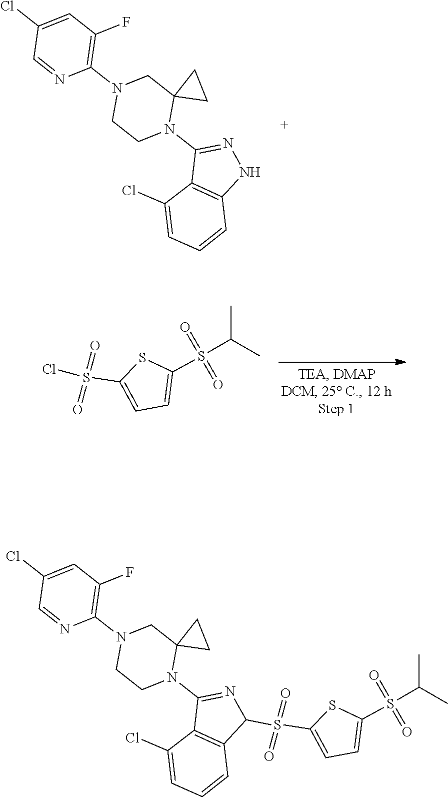 [SMILES: Fc1cc(Cl)cnc1N1CCN(c2n[nH]c3cccc(Cl)c23)C2(CC2)C1 ; CC(C)S(=O)(=O)c1ccc(S(=O)(=O)Cl)s1 ; CC(C)S(=O)(=O)c1ccc(S(=O)(=O)C2N=C(N3CCN(c4ncc(Cl)cc4F)CC34CC4)c3c(Cl)cccc32)s1]

Step 1: To a solution of 4-chloro-3-[7-(5-chloro-3-fluoro-2-pyridyl)-4,7-diazaspiro[2.5]octan-4-yl]-1H-indazole (50 mg, 127 μmol, 1 eq) and 5-isopropylsulfonylthiophene-2-sulfonyl chloride (73.6 mg, 255 μmol, 2 eq) in DCM (1 mL) was added TEA (25.8 mg, 255 μmol, 35.5 μL, 2 eq) and DMAP (1.56 mg, 12.8 μmol, 0.1 eq). The mixture was stirred at 25° C. for 12 hours. LC-MS showed 4-chloro-3-[7-(5-chloro-3-fluoro-2-pyridyl)-4, 7-diazaspiro [2.5]octan-4-yl]-1H-indazole was consumed completely and one main peak with desired mass was detected. The reaction mixture was diluted with H₂O (3 mL) and extracted with DCM (4 mL). The combined organic layers were washed with brine (4 mL), dried over anhydrous Na₂SO₄, filtered and concentrated under reduced pressure to give a residue. The residue was purified by prep-HPLC (neutral condition: column: Waters Xbridge Prep OBD C18 150*40 mm*10 μm; mobile phase: [water (NH₄HCO₃)-ACN]; B %: 65%-95%, 8 min) to give desired 4-chloro-3-[7-(5-chloro-3-fluoro-2-pyridyl)-4,7-diazaspiro[2.5]octan-4-yl]-1-[(5-isopropylsulfonyl-2-thienyl)sulfonyl]indazole (10 mg, 15.5 μmol, 12.2% yield, 100.00% purity) as a pale yellow solid. ¹H NMR (400 MHz, DMSO-d₆) δ 8.09 (d, J=2.00 Hz, 1H) 8.02 (d, J=8.50 Hz, 1H) 7.84 (s, 1H) 7.83 (s, 1H) 7.75 (d, J=4.13 Hz, 1H) 7.68 (s, 1H) 7.53 (d, J=7.75 Hz, 1H) 3.67 (br s, 1H) 3.51-3.60 (m, 6H) 1.12 (d, J=6.75 Hz, 6H) 0.81 (br s, 2H) 0.50 (br s, 2H). HPLC: 100.00% (220 nm), 100.00% (215 nm), 100.00% (254 nm). MS (ESI): mass calcd. For C₂₅H₂₄C₁₂FN₅O₄S₃ 643.04 m/z found 644.2 [M+H]⁺.

### Compound 31: 4-chloro-3-(7-(3, 5-difluoropyridin-2-yl)-4, 7-diazaspiro [2.5]octan-4-yl)-7-fluoro-1-((5-(isopropylsulfonyl) thiophen-2-yl) sulfonyl)-1H-indazole

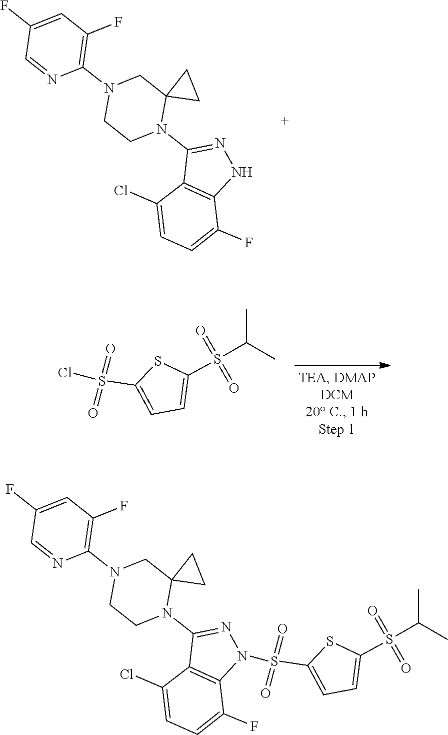 [SMILES: Fc1cnc(N2CCN(c3n[nH]c4c(F)ccc(Cl)c34)C3(CC3)C2)c(F)c1 ; CC(C)S(=O)(=O)c1ccc(S(=O)(=O)Cl)s1 ; CC(C)S(=O)(=O)c1ccc(S(=O)(=O)n2nc(N3CCN(c4ncc(F)cc4F)CC34CC4)c3c(Cl)ccc(F)c32)s1]

Step 1: To a solution of 4-chloro-3-[7-(3, 5-difluoro-2-pyridyl)-4, 7-diazaspiro [2.5]octan-4-yl]-7-fluoro-1H-indazole (100 mg, 254 μmol, 1 eq) and 5-isopropylsulfonylthiophene-2-sulfonyl chloride (88.0 mg, 305 μmol, 1.2 eq) in DCM (2 mL) was added TEA (51.4 mg, 508 μmol, 70.7 μL, 2 eq) and DMAP (3.10 mg, 25.4 μmol, 0.1 eq). The mixture was stirred at 20° C. for 1 hour. LC-MS showed 4-chloro-3-[7-(3, 5-difluoro-2-pyridyl)-4, 7-diazaspiro [2.5]octan-4-yl]-7-fluoro-1H-indazole was consumed completely and desired mass was detected. The crude was added H₂O (10 mL), and extracted with EtOAc (15 mL*3). The combined organic layers were washed with brine 10 mL, dried over Na₂SO₄, filtered and concentrated under reduced pressure to give a residue. The residue was purified by prep-HPLC (neutral condition, column: Waters Xbridge Prep OBD C18 150*40 mm*10 μm; mobile phase: [water(NH₄HCO₃)-ACN]; B %: 65%-95%, 8 min) to give desired 4-chloro-3-(7-(3, 5-difluoropyridin-2-yl)-4, 7-diazaspiro [2.5]octan-4-yl)-7-fluoro-1-((5-(isopropylsulfonyl) thiophen-2-yl) sulfonyl)-1H-indazole (35.2 mg, 54.2 μmol, 21.3% yield, 99.39% purity) as a white solid. ¹H NMR (400 MHz, DMSO-d₆) δ 8.14 (d, J=2.3 Hz, 1H), 8.07-8.02 (m, 1H), 7.87 (d, J=4.2 Hz, 1H), 7.85-7.82 (m, 1H), 7.81-7.76 (m, 2H), 3.69 (br s, 2H), 3.63-3.54 (m, 2H), 3.34 (br s, 2H), 3.32-3.26 (m, 1H), 1.15 (d, J=6.8 Hz, 6H), 0.85-0.80 (m, 2H), 0.53 (br s, 2H). HPLC: 99.39% (220 nm), 99.49% (215 nm), 99.67% (254 nm). MS (ESI): mass calcd. For C₂₅H₂₃ClF₃N₅O₄S₃ 645.06 m/z found 646.1 [M+H]⁺.

### Compound 32: 3-[7-(3, 5-difluoro-2-pyridyl)-4, 7-diazaspiro [2.5]octan-4-yl]-4, 7-difluoro-1-[(5-isopropylsulfonyl-2-thienyl) sulfonyl]indazole

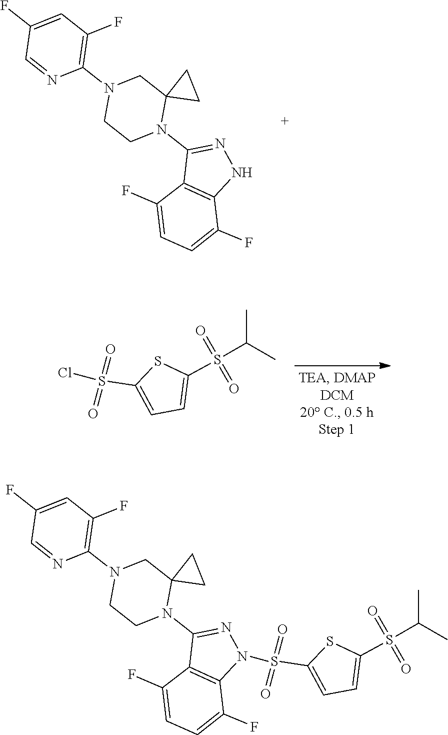 [SMILES: Fc1cnc(N2CCN(c3n[nH]c4c(F)ccc(F)c34)C3(CC3)C2)c(F)c1 ; CC(C)S(=O)(=O)c1ccc(S(=O)(=O)Cl)s1 ; CC(C)S(=O)(=O)c1ccc(S(=O)(=O)n2nc(N3CCN(c4ncc(F)cc4F)CC34CC4)c3c(F)ccc(F)c32)s1]

Step 1: A mixture of 3-[7-(3,5-difluoro-2-pyridyl)-4,7-diazaspiro[2.5]octan-4-yl]-4,7-difluoro-1H-indazole (30 mg, 79.5 μmol, 1 eq) and 5-isopropylsulfonylthiophene-2-sulfonyl chloride (45.9 mg, 159 μmol, 2 eq) in DCM (1 mL) was added TEA (24.1 mg, 239 μmol, 33.2 μL, 3 eq) and DMAP (971 μg, 7.95 μmol, 0.1 eq), and then the mixture was stirred at 20° C. for 0.5 hour. LC-MS showed 3-[7-(3, 5-difluoro-2-pyridyl)-4, 7-diazaspiro [2.5]octan-4-yl]-4, 7-difluoro-1H-indazole was consumed completely and one main peak with desired mass was detected. The residue was diluted with H₂O (10 mL) and extracted with EtOAc (5 mL*3). The combined organic layers were washed with brine (10 mL*1), dried over Na₂SO₄, filtered and concentrated under reduced pressure to give a residue. The residue was purified by prep-TLC (SiO₂, petroleum ether/EtOAc=3/1) to give desired 3-[7-(3, 5-difluoro-2-pyridyl)-4, 7-diazaspiro [2.5]octan-4-yl]-4, 7-difluoro-1-[(5-isopropylsulfonyl-2-thienyl) sulfonyl]indazole (14.7 mg, 23.1 μmol, 29.0% yield, 98.76% purity) as a yellow solid. ¹H NMR (400 MHz, DMSO-d₆) δ 8.11-8.08 (m, 1H), 7.82 (br d, J=2.5 Hz, 1H), 7.78-7.76 (m, 1H), 7.75-7.72 (m, 1H), 7.67-7.59 (m, 1H), 7.34-7.27 (m, 1H), 3.74-3.67 (m, 2H), 3.60 (br s, 3H), 3.27-3.15 (m, 2H), 1.15-1.08 (m, 6H), 0.89-0.82 (m, 2H), 0.65-0.58 (m, 2H). HPLC: 98.76% (220 nm), 98.08% (215 nm), 99.32% (254 nm). MS (ESI): mass calcd. For C₂₅H₂₃F₄N₅O₄S₃ 629.08, m/z found 630.0 [M+H]⁺.

### Compound 33: 3-[7-(5-chloro-3-fluoro-2-pyridyl)-4, 7-diazaspiro [2.5]octan-4-yl]-4, 7-difluoro-1-[(5-isopropylsulfonyl-2-thienyl) sulfonyl]indazole

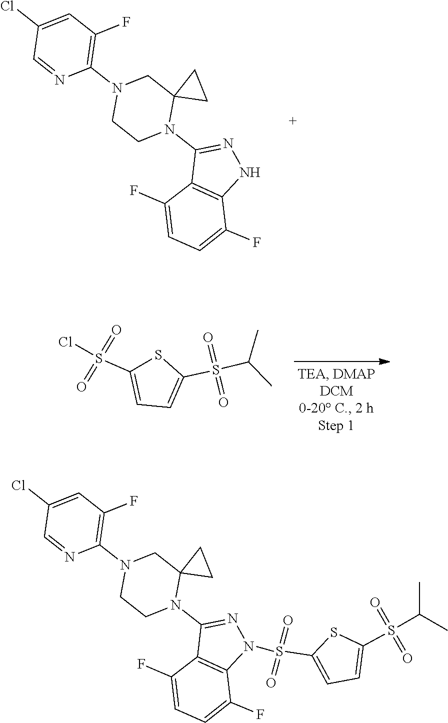 [SMILES: Fc1cc(Cl)cnc1N1CCN(c2n[nH]c3c(F)ccc(F)c23)C2(CC2)C1 ; CC(C)S(=O)(=O)c1ccc(S(=O)(=O)Cl)s1 ; CC(C)S(=O)(=O)c1ccc(S(=O)(=O)n2nc(N3CCN(c4ncc(Cl)cc4F)CC34CC4)c3c(F)ccc(F)c32)s1]

Step 1: To a solution of 3-[7-(5-chloro-3-fluoro-2-pyridyl)-4, 7-diazaspiro [2.5]octan-4-yl]-4, 7-difluoro-1H-indazole (50 mg, 127 μmol, 1 eq) and 5-isopropylsulfonylthiophene-2-sulfonyl chloride (73.3 mg, 254 μmol, 2 eq) in DCM (1 mL) was added DMAP (1.55 mg, 12.7 μmol, 0.1 eq) and TEA (38.5 mg, 381 μmol, 53.0 μL, 3 eq) at 0° C. The mixture was stirred at 20° C. for 2 hours. LC-MS showed 3-[7-(5-chloro-3-fluoro-2-pyridyl)-4, 7-diazaspiro [2.5]octan-4-yl]-4, 7-difluoro-1H-indazole was consumed completely and desired mass was detected. The residue was diluted with water (20 mL) and extracted with DCM (20 mL). The combined organic layers were washed with brine (20 mL), dried over Na₂SO₄, filtered and concentrated under reduced pressure to give a residue. The residue was purified by prep-HPLC (neutral condition; column: Waters Xbridge Prep OBD C18 150*40 mm*10 μm; mobile phase: [water(NH₄HCO₃)-ACN]; B %: 65%-95%, 8 min) to give desired 3-[7-(5-chloro-3-fluoro-2-pyridyl)-4, 7-diazaspiro [2.5]octan-4-yl]-4, 7-difluoro-1-[(5-isopropylsulfonyl-2-thienyl) sulfonyl]indazole (19.7 mg, 30.4 μmol, 24.0% yield, 99.79% purity) as an off-white solid. ¹H NMR (400 MHz, DMSO-d₆) δ 8.10 (d, J=1.6 Hz, 1H), 7.86 (dd, J=2.1, 12.9 Hz, 1H), 7.82-7.80 (m, 1H), 7.79-7.76 (m, 1H), 7.66 (ddd, J=3.6, 8.9, 10.8 Hz, 1H), 7.34 (dt, J=2.5, 9.2 Hz, 1H), 3.72 (br t, J=4.6 Hz, 2H), 3.64-3.53 (m, 3H), 3.35 (br s, 2H), 1.14 (d, J=6.9 Hz, 6H), 0.89 (br s, 2H), 0.63 (br s, 2H). HPLC: 99.79% (220 nm), 99.76% (215 nm), 100.0% (254 nm). MS (ESI): mass calcd. For C₂₅H₂₃ClF₃N₅O₄S₃ 645.06, m/z found 646.0 [M+H]⁺.

### Compound 34: 4, 7-difluoro-3-(7-(5-fluoropyrimidin-2-yl)-4, 7-diazaspiro [2.5]octan-4-yl)-1-((5-(isopropylsulfonyl) thiophen-2-yl) sulfonyl)-1H-indazole

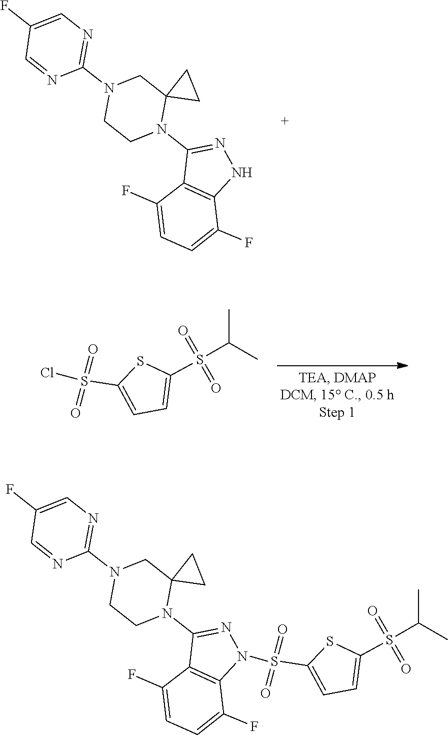 [SMILES: Fc1cnc(N2CCN(c3n[nH]c4c(F)ccc(F)c34)C3(CC3)C2)nc1 ; CC(C)S(=O)(=O)c1ccc(S(=O)(=O)Cl)s1 ; CC(C)S(=O)(=O)c1ccc(S(=O)(=O)n2nc(N3CCN(c4ncc(F)cn4)CC34CC4)c3c(F)ccc(F)c32)s1]

Step 1: To a solution of 4, 7-difluoro-3-[7-(5-fluoropyrimidin-2-yl)-4, 7-diazaspiro [2.5]octan-4-yl]-1H-indazole (50 mg, 139 μmol, 1 eq) and 5-isopropyl sulfonyl thiophene-2-sulfonyl chloride (60.1 mg, 208 μmol, 1.5 eq) in DCM (1 mL) was added TEA (28.1 mg, 278 μmol, 38.6 μL, 2 eq) and DMAP (1.70 mg, 13.9 μmol, 0.1 eq). The mixture was stirred at 15° C. for 0.5 hour. LC-MS showed 4, 7-difluoro-3-[7-(5-fluoropyrimidin-2-yl)-4, 7-diazaspiro [2.5]octan-4-yl]-1H-indazole was consumed completely and desired mass was detected. The crude was added H₂O (10 mL), and extracted with EtOAc (15 mL*3). The combined organic layers were washed with brine (10 mL), dried over Na₂SO₄, filtered and concentrated under reduced pressure to give a residue. The residue was purified by prep-HPLC (neutral condition, column: Phenomenex C18 75*30 mm*3 m; mobile phase: [water(NH₄HCO₃)-ACN]; B %: 25%-60%, 8 min) to give desired 4, 7-difluoro-3-(7-(5-fluoropyrimidin-2-yl)-4, 7-diazaspiro [2.5]octan-4-yl)-1-((5-(isopropylsulfonyl) thiophen-2-yl) sulfonyl)-1H-indazole (10.8 mg, 17.5 μmol, 12.6% yield, 99.46% purity) as a white solid. ¹H NMR (400 MHz, DMSO-d₆) δ 8.46 (s, 2H), 7.79 (q, J=4.1 Hz, 2H), 7.65 (ddd, J=3.6, 8.9, 10.9 Hz, 1H), 7.36-7.29 (m, 1H), 3.90 (s, 2H), 3.64 (br s, 2H), 3.60-3.54 (m, 1H), 3.32 (br s, 2H), 1.13 (d, J=6.8 Hz, 6H), 0.88 (s, 2H), 0.66-0.58 (m, 2H). HPLC: 99.46% (220 nm), 99.48% (215 nm), 99.69% (254 nm). MS (ESI): mass calcd. For C₂₄H₂₃F₃N₆O₄S₃ 612.09 m/z found 613.1 [M+H]⁺.

### Compound 35: 4-chloro-3-(7-(5-chloro-3-fluoropyridin-2-yl)-4,7-diazaspiro[2.5]octan-4-yl)-6-fluoro-1-((5-(isopropylsulfonyl)thiophen-2-yl)sulfonyl)-1H-indazole

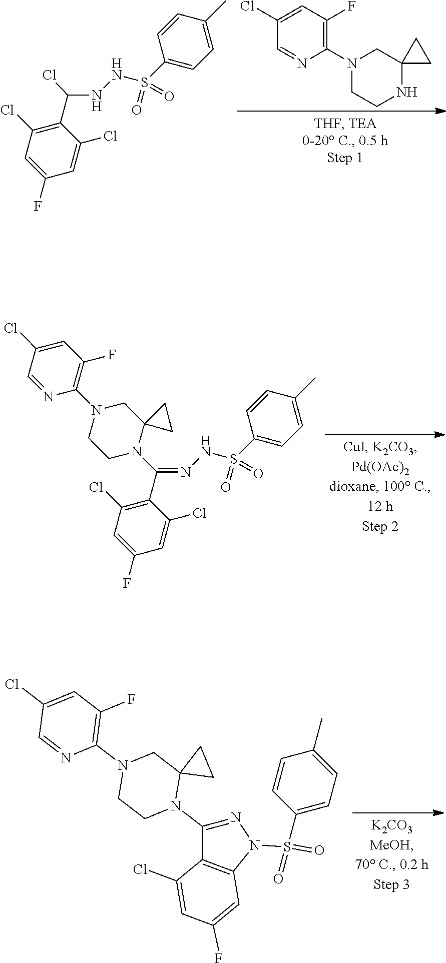

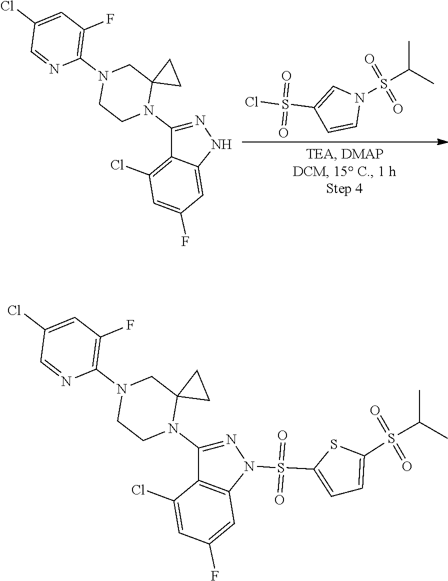 [SMILES: Fc1cc(Cl)c2c(N3CCN(c4ncc(Cl)cc4F)CC34CC4)n[nH]c2c1 ; CC(C)S(=O)(=O)n1ccc(S(=O)(=O)Cl)c1 ; CC(C)S(=O)(=O)c1ccc(S(=O)(=O)n2nc(N3CCN(c4ncc(Cl)cc4F)CC34CC4)c3c(Cl)cc(F)cc32)s1]

Step 1: To a solution of (1Z)-2,6-dichloro-4-fluoro-N-(p-tolylsulfonyl)benzohydrazonoyl chloride (1.14 g, 2.88 mmol, 1 eq) and 7-(5-chloro-3-fluoro-2-pyridyl)-4,7-diazaspiro[2.5]octane (800 mg, 2.88 mmol, 1 eq, HCl) in THF (10 mL) was added TEA (582 mg, 5.75 mmol, 801 μL, 2 eq). The mixture was stirred at 15° C. for 0.5 hour. LC-MS showed 2, 6-dichloro-4-fluoro-N′-(p-tolylsulfonyl) benzohydrazide was consumed completely and desired mass was detected. The crude was added H₂O (20 mL) and extracted with EtOAc (15 mL*3). The combined organic layers were washed with brine (20 mL), dried over anhydrous Na₂SO₄, filtered and concentrated under reduced pressure to give desired (Z)—N′-((7-(5-chloro-3-fluoropyridin-2-yl)-4, 7-diazaspiro [2.5]octan-4-yl) (2, 6-dichloro-4-fluorophenyl) methylene)-4-methylbenzenesulfonohydrazide (1.7 g, crude) as a yellow solid. MS (ESI): mass calcd. For C₂₅H₂₂Cl₃F₂NO₂S 599.05, m/z found 600.1 [M+H]⁺.

Step 2: A mixture of N—[(Z)—[[7-(5-chloro-3-fluoro-2-pyridyl)-4,7-diazaspiro[2.5]octan-4-yl]-(2,6-dichloro-4-fluoro-phenyl)methylene]amino]-4-methyl-benzenesulfonamide (1.7 g, 2.83 mmol, 1 eq), CuI (53.9 mg, 283 μmol, 0.1 eq), K₂CO₃ (978 mg, 7.07 mmol, 2.5 eq) and Pd(OAc)₂ (127 mg, 566 μmol, 0.2 eq) in dioxane (20 mL) was degassed and purged with N₂ for 3 times, and then the mixture was stirred at 100° C. for 12 hours under the atmosphere of nitrogen. LC-MS showed N—[(Z)—[[7-(5-chloro-3-fluoro-2-pyridyl)-4, 7-diazaspiro [2.5]octan-4-yl]-(2, 6-dichloro-4-fluoro-phenyl) methylene]amino]-4-methyl-benzenesulfonamide was consumed completely and desired mass was detected. The crude was added H₂O (40 mL) and extracted with EtOAc (30 mL*3). The combined organic layers were washed with brine (40 mL), dried over anhydrous Na₂SO₄, filtered and concentrated under reduced pressure to give desired 4-chloro-3-(7-(5-chloro-3-fluoropyridin-2-yl)-4, 7-diazaspiro [2.5]octan-4-yl)-6-fluoro-1-tosyl-1H-indazole (1.6 g, crude) as a yellow solid. MS (ESI): mass calcd. For C₂₅H₂₁C₁₂F₂N₅O₂S 563.08, m/z found 564.1 [M+H]⁺.

Step 3: To a solution of 4-chloro-3-[7-(5-chloro-3-fluoro-2-pyridyl)-4, 7-diazaspiro [2.5]octan-4-yl]-6-fluoro-1-(p-tolylsulfonyl) indazole (1.6 g, 2.83 mmol, 1 eq) in MeOH (20 mL) was added K₂CO₃ (1.96 g, 14.2 mmol, 5 eq). The mixture was stirred at 70° C. for 0.2 hour. LC-MS showed 4-chloro-3-[7-(5-chloro-3-fluoro-2-pyridyl)-4, 7-diazaspiro [2.5]octan-4-yl]-6-fluoro-1-(p-tolylsulfonyl) indazole was consumed completely and desired mass was detected. The reaction mixture was concentrated under reduced pressure to give a residue. The crude was added H₂O (40 mL) and extracted with EtOAc (30 mL*3). The combined organic layers were washed with brine (40 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜35% EtOAc/petroleum ethergradient @100 mL/min) to give desired 4-chloro-3-(7-(5-chloro-3-fluoropyridin-2-yl)-4, 7-diazaspiro [2.5]octan-4-yl)-6-fluoro-1H-indazole (300 mg, 731.27 μmol, 25.80% yield) as a yellow oil. MS (ESI): mass calcd. For C₁₈H₁₅C₁₂F₂N₅ 409.07, m/z found 410.0 [M+H]⁺.

Step 4: To a solution of 4-chloro-3-[7-(5-chloro-3-fluoro-2-pyridyl)-4,7-diazaspiro[2.5]octan-4-yl]-6-fluoro-1H-indazole (50 mg, 123 μmol, 1 eq) and 5-isopropylsulfonylthiophene-2-sulfonyl chloride (45.8 mg, 158 μmol, 1.3 eq) in DCM (1 mL) was added TEA (24.7 mg, 244 μmol, 2 eq) and DMAP (1.49 mg, 12.2 μmol, 0.1 eq). The mixture was stirred at 15° C. for 1 hour. LC-MS showed 4-chloro-3-[7-(5-chloro-3-fluoro-2-pyridyl)-4, 7-diazaspiro [2.5]octan-4-yl]-6-fluoro-1H-indazole was consumed completely and desired mass was detected. The crude was added H₂O (10 mL), and extracted with EtOAc (15 mL*3). The combined organic layers were washed with brine 10 mL, dried over Na₂SO₄, filtered and concentrated under reduced pressure to give a residue. The residue was purified by prep-TLC (petroleum ether/EtOAc=3/1) to give desired 4-chloro-3-(7-(5-chloro-3-fluoropyridin-2-yl)-4, 7-diazaspiro [2.5]octan-4-yl)-6-fluoro-1-((5-(isopropylsulfonyl) thiophen-2-yl) sulfonyl)-1H-indazole (5.2 mg, 7.83 μmol, 6.43% yield, 99.82% purity) as a white solid. ¹H NMR (400 MHz, DMSO-d6) δ=8.14 (s, 1H), 8.03-7.98 (m, 1H), 7.92-7.83 (m, 3H), 7.67 (br d, J=8.9 Hz, 1H), 3.75-3.66 (m, 4H), 3.64-3.60 (m, 1H), 3.48-3.41 (m, 2H), 1.20 (br d, J=6.5 Hz, 6H), 0.87 (br s, 2H), 0.56 (br s, 2H). HPLC: 99.81% (220 nm), 99.27% (215 nm), 100.00 (254 nm). MS (ESI): mass calcd. For C₂₅H₂₄C₁₂F₂N₆O₄S₂ 661.03 m/z found 662.1 [M+H]⁺.

### Compound 36: 4-chloro-3-(7-(5-chloropyrimidin-2-yl)-4, 7-diazaspiro [2.5]octan-4-yl)-6-fluoro-1-((5-(isopropylsulfonyl) thiophen-2-yl) sulfonyl)-1H-indazole

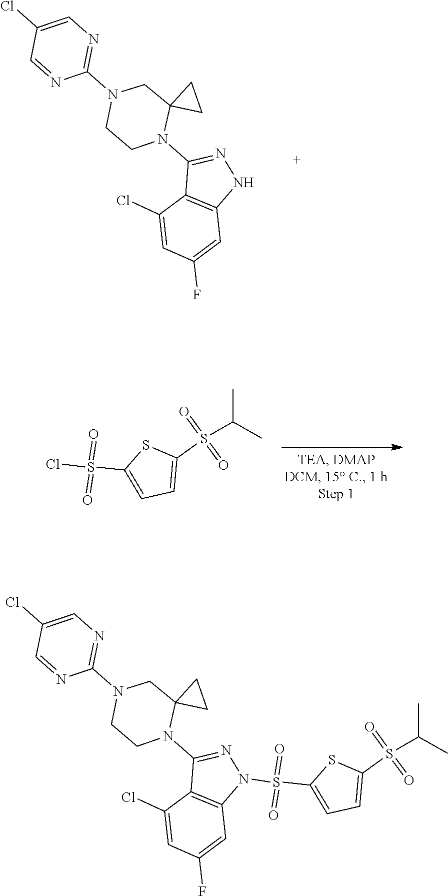 [SMILES: Fc1cc(Cl)c2c(N3CCN(c4ncc(Cl)cn4)CC34CC4)n[nH]c2c1 ; CC(C)S(=O)(=O)c1ccc(S(=O)(=O)Cl)s1 ; CC(C)S(=O)(=O)c1ccc(S(=O)(=O)n2nc(N3CCN(c4ncc(Cl)cn4)CC34CC4)c3c(Cl)cc(F)cc32)s1]

Step 1: To a solution of 4-chloro-3-[7-(5-chloropyrimidin-2-yl)-4, 7-diazaspiro [2.5]octan-4-yl]-6-fluoro-1H-indazole (30 mg, 76.3 μmol, 1 eq) and 5-isopropylsulfonylthiophene-2-sulfonyl chloride (26.4 mg, 91.6 μmol, 1.2 eq) in DCM (1 mL) was added TEA (15.4 mg, 153 μmol, 21.2 μL, 2 eq) and DMAP (932 μg, 7.63 μmol, 0.1 eq). The mixture was stirred at 15° C. for 1 hour. LC-MS showed 4-chloro-3-[7-(5-chloropyrimidin-2-yl)-4, 7-diazaspiro [2.5]octan-4-yl]-6-fluoro-1H-indazole was consumed completely and desired mass was detected. The reaction mixture was concentrated under reduced pressure to give a residue. The residue was purified by prep-HPLC (FA condition, column: Phenomenex Luna C18 75*30 mm*3 μm; mobile phase: [water (FA)-ACN]; B %: 60%-80%, 8 min) to give desired 4-chloro-3-(7-(5-chloropyrimidin-2-yl)-4, 7-diazaspiro [2.5]octan-4-yl)-6-fluoro-1-((5-(isopropylsulfonyl) thiophen-2-yl) sulfonyl)-1H-indazole (4.5 mg, 6.83 μmol, 8.95% yield, 98.00% purity) as a white solid. ¹H NMR (400 MHz, DMSO-d₆) δ 8.44 (s, 2H), 7.98-7.94 (m, 1H), 7.84-7.77 (m, 2H), 7.66-7.55 (m, 1H), 3.99-3.94 (m, 2H), 3.74-3.66 (m, 2H), 3.61-3.54 (m, 3H), 1.17-1.12 (m, 6H), 0.91-0.81 (m, 2H), 0.65-0.49 (m, 2H). HPLC: 93.00% (220 nm), 92.20% (215 nm), 98.00 (254 nm). MS (ESI): mass calcd. For C₂₄H₂₃C₁₂FN₆O₄S₃ 644.03 m/z found 645.1 [M+H]⁺.

### Compound 37: 4-chloro-3-[7-(3, 5-difluoro-2-pyridyl)-4, 7-diazaspiro [2.5]octan-4-yl]-6-fluoro-1-[(5-isopropylsulfonyl-2-thienyl) sulfonyl]indazole

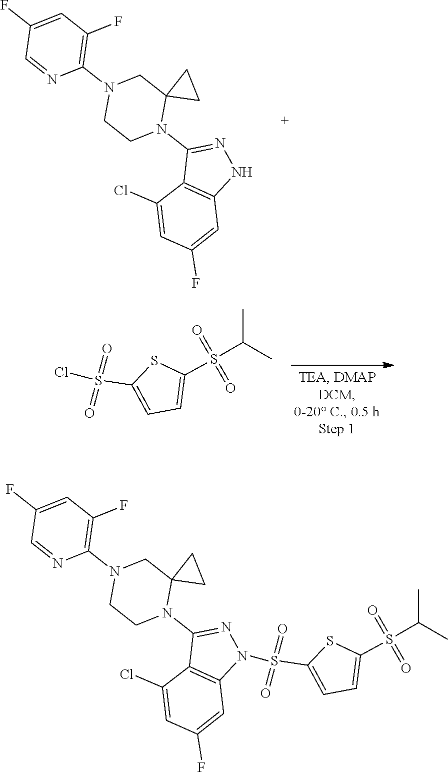 [SMILES: Fc1cnc(N2CCN(c3n[nH]c4cc(F)cc(Cl)c34)C3(CC3)C2)c(F)c1 ; CC(C)S(=O)(=O)c1ccc(S(=O)(=O)Cl)s1 ; CC(C)S(=O)(=O)c1ccc(S(=O)(=O)n2nc(N3CCN(c4ncc(F)cc4F)CC34CC4)c3c(Cl)cc(F)cc32)s1]

Step 1: To a solution of 5-isopropylsulfonylthiophene-2-sulfonyl chloride (147 mg, 508 μmol, 2 eq) and 4-chloro-3-[7-(3, 5-difluoro-2-pyridyl)-4, 7-diazaspiro [2.5]octan-4-yl]-6-fluoro-1H-indazole (100 mg, 254 μmol, 1 eq) in DCM (1 mL) was added TEA (51.4 mg, 508 μmol, 70.7 μL, 2 eq) and DMAP (3.10 mg, 25.4 μmol, 0.1 eq) at 0° C. The mixture was stirred at 20° C. for 0.5 hour. LC-MS showed 5-isopropylsulfonylthiophene-2-sulfonyl chloride was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-TLC (SiO₂, petroleum ether:EtOAc=3:1) to give desired 4-chloro-3-[7-(3, 5-difluoro-2-pyridyl)-4, 7-diazaspiro [2.5]octan-4-yl]-6-fluoro-1-[(5-isopropylsulfonyl-2-thienyl) sulfonyl]indazole (38.54 mg, 53.86 μmol, 90.29% purity) as a white solid. ¹H NMR (400 MHz, DMSO-d₆) δ 8.12-8.10 (m, 1H), 7.96-7.93 (m, 1H), 7.85-7.76 (m, 3H), 7.64-7.59 (m, 1H), 3.69-3.62 (m, 2H), 3.61-3.51 (m, 2H), 3.35-3.32 (m, 1H), 3.31-3.25 (m, 2H), 1.18-1.10 (m, 6H), 0.84-0.75 (m, 2H), 0.55-0.46 (m, 2H). HPLC: 90.29% (220 nm), 91.10% (215 nm), 94.52% (254 nm). MS (ESI): mass calcd. For C₂₅H₂₃ClF₃N₅O₄S₃ 645.06, m/z found 646.1 [M+H]⁺.

### Compound 38: 4-chloro-6-fluoro-3-(7-(5-fluoropyrimidin-2-yl)-4, 7-diazaspiro [2.5]octan-4-yl)-1-((5-(isopropylsulfonyl) thiophen-2-yl) sulfonyl)-1H-indazole

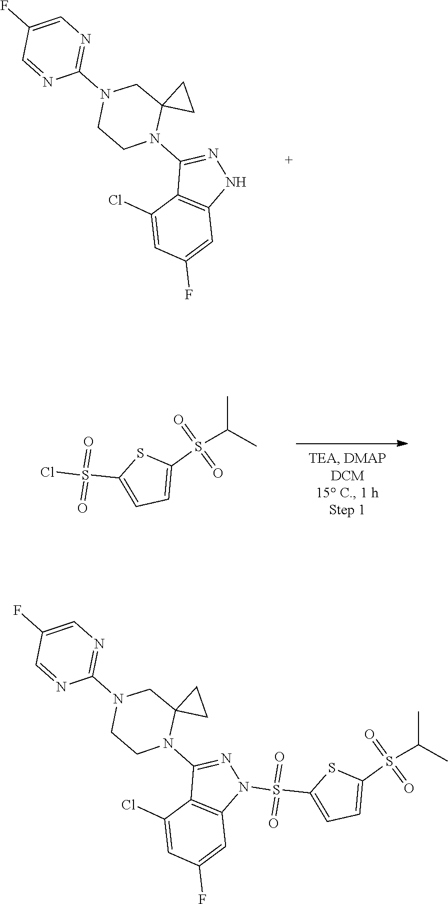 [SMILES: Fc1cnc(N2CCN(c3n[nH]c4cc(F)cc(Cl)c34)C3(CC3)C2)nc1 ; CC(C)S(=O)(=O)c1ccc(S(=O)(=O)Cl)s1 ; CC(C)S(=O)(=O)c1ccc(S(=O)(=O)n2nc(N3CCN(c4ncc(F)cn4)CC34CC4)c3c(Cl)cc(F)cc32)s1]

Step 1: To a solution of 4-chloro-6-fluoro-3-[7-(5-fluoropyrimidin-2-yl)-4, 7 diazaspiro [2.5]octan-4-yl]-1H-indazole (150 mg, 398 μmol, 1 eq) and 5-isopropylsulfonylthiophene-2-sulfonyl chloride (172 mg, 597 μmol, 1.5 eq) in DCM (2 mL) was added TEA (80.6 mg, 796 μmol, 111 μL, 2 eq) and DMAP (4.86 mg, 39.8 μmol, 0.1 eq). The mixture was stirred at 15° C. for 1 hour. LC-MS showed 4-chloro-6-fluoro-3-[7-(5-fluoropyrimidin-2-yl)-4, 7-diazaspiro [2.5]octan-4-yl]-1H-indazole was consumed completely and desired mass was detected. The reaction mixture was concentrated under reduced pressure to give a residue. The crude product was purified by reversed-phase HPLC (column: C18 20-35 μm 100A 40 g; mobile phase: [water-ACN]; B %: 0%-25% @50 mL/min) to give desired 4-chloro-6-fluoro-3-(7-(5-fluoropyrimidin-2-yl)-4, 7-diazaspiro [2.5]octan-4-yl)-1-((5-(isopropylsulfonyl) thiophen-2-yl) sulfonyl)-1H-indazole (48.7 mg, 74.6 μmol, 18.7% yield, 96.34% purity) as a white solid. ¹H NMR (400 MHz, DMSO-d₆) δ 8.48 (s, 2H), 7.98 (d, J=4.1 Hz, 1H), 7.85-7.78 (m, 2H), 7.64 (dd, J=1.8, 9.1 Hz, 1H), 3.95 (s, 2H), 3.79-3.52 (m, 5H), 1.15 (d, J=6.8 Hz, 6H), 0.86-0.80 (m, 2H), 0.54-0.47 (m, 2H). HPLC: 96.34% (220 nm), 97.76% (215 nm), 99.57 (254 nm). MS (ESI): mass calcd. For C₂₄H₂₃ClF₂N₆O₄S₃ 628.06 m/z found 629.1 [M+H]⁺.

### Compound 39: 5-chloro-2-(4-(4-chloro-6-fluoro-1-((5-(isopropylsulfonyl) thiophen-2-yl) sulfonyl)-1H-indazol-3-yl)-4, 7-diazaspiro [2.5]octan-7-yl) thiazole

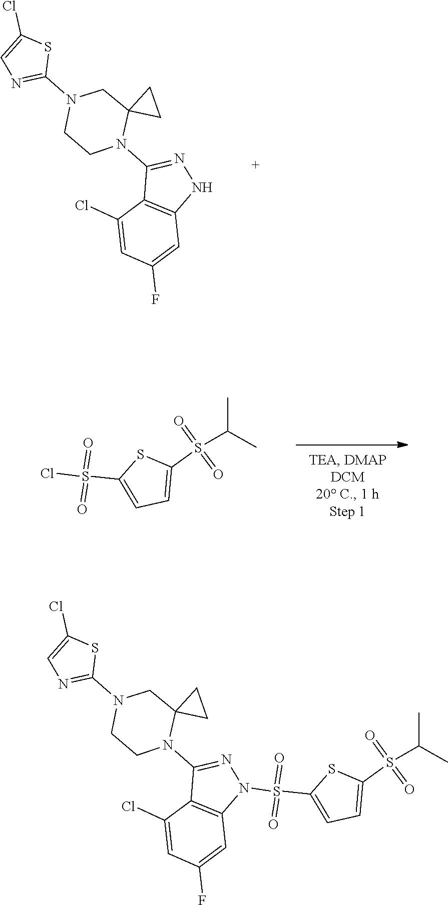 [SMILES: Fc1cc(Cl)c2c(N3CCN(c4ncc(Cl)s4)CC34CC4)n[nH]c2c1 ; CC(C)S(=O)(=O)c1ccc(S(=O)(=O)Cl)s1 ; CC(C)S(=O)(=O)c1ccc(S(=O)(=O)n2nc(N3CCN(c4ncc(Cl)s4)CC34CC4)c3c(Cl)cc(F)cc32)s1]

Step 1: To the solution of 5-chloro-2-[4-(4-chloro-6-fluoro-1H-indazol-3-yl)-4,7-diazaspiro[2.5]octan-7-yl]thiazole (30 mg, 75.32 μmol, 1 eq), TEA (22.9 mg, 226 μmol, 31.5 μL, 3 eq) and DMAP (920 μg, 7.53 μmol, 0.1 eq) in DCM (2 mL) was added 5-isopropylsulfonylthiophene-2-sulfonyl chloride (65.3 mg, 226 μmol, 3 eq) at 20° C. and the solution was stirred at 20° C. for 1 hour. LC-MS showed 5-chloro-2-[4-(4-chloro-6-fluoro-1H-indazol-3-yl)-4, 7-diazaspiro [2.5]octan-7-yl]thiazole was consumed completely desired mass was detected. The residue was purified by prep-TLC (SiO₂, petroleum ether/EtOAc=3/1) to give desired 5-chloro-2-(4-(4-chloro-6-fluoro-1-((5-(isopropylsulfonyl) thiophen-2-yl) sulfonyl)-1H-indazol-3-yl)-4, 7-diazaspiro [2.5]octan-7-yl) thiazole (5.2 mg, 7.87 μmol, 10.45% yield, 98.47% purity) as a white solid. ¹H NMR (400 MHz, DMSO-d₆) δ 7.97 (d, J=4.0 Hz, 1H), 7.83-7.78 (m, 2H), 7.63 (dd, J=1.8, 9.2 Hz, 1H), 7.18 (s, 1H), 3.69-3.54 (m, 5H), 3.41-3.35 (m, 2H), 1.16 (d, J=6.8 Hz, 6H), 0.88 (br s, 2H), 0.53 (br s, 2H). HPLC: 98.47% (220 nm), 97.84% (215 nm), 99.14% (254 nm). MS (ESI): mass calcd. For C₂₃H₂₂C₁₂FN₅O₄S₄ 648.9, m/z found 650.1 [M+H]⁺.

### Compound 40: 3-(7-(3, 5-difluoropyridin-2-yl)-4, 7-diazaspiro [2.5]octan-4-yl)-4, 6-difluoro-1-((5-(isopropylsulfonyl) thiophen-2-yl) sulfonyl)-1H-indazole

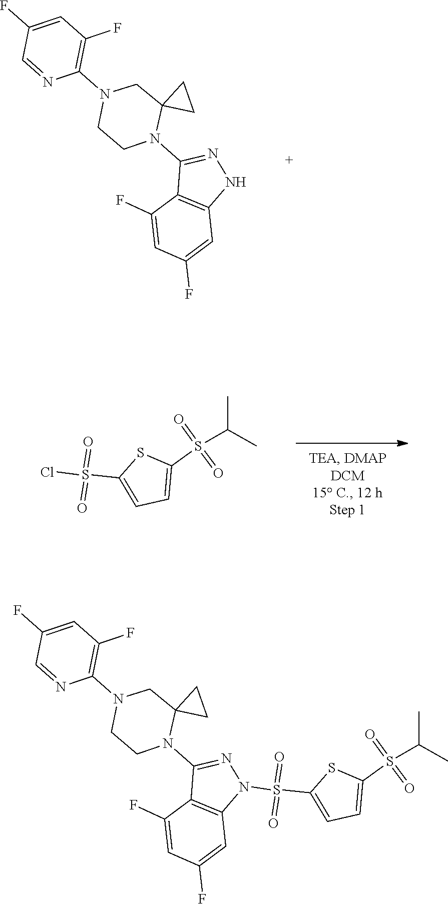 [SMILES: Fc1cnc(N2CCN(c3n[nH]c4cc(F)cc(F)c34)C3(CC3)C2)c(F)c1 ; CC(C)S(=O)(=O)c1ccc(S(=O)(=O)Cl)s1 ; CC(C)S(=O)(=O)c1ccc(S(=O)(=O)n2nc(N3CCN(c4ncc(F)cc4F)CC34CC4)c3c(F)cc(F)cc32)s1]

Step 1: To a solution of 3-[7-(3,5-difluoro-2-pyridyl)-4,7-diazaspiro[2.5]octan-4-yl]-4,6-difluoro-1H-indazole (30 mg, 79.5 μmol, 1 eq) and 5-isopropylsulfonylthiophene-2-sulfonyl chloride (34.4 mg, 119 μmol, 1.5 eq) in DCM (1 mL) was added TEA (16.1 mg, 159 μmol, 22.1 μL, 2 eq) and DMAP (971 μg, 7.95 μmol, 0.1 eq). The mixture was stirred at 15° C. for 12 hours. LC-MS showed 3-[7-(3,5-difluoro-2-pyridyl)-4,7-diazaspiro[2.5]octan-4-yl]-4,6-difluoro-1H-indazole was consumed completely desired mass was detected. The reaction mixture was concentrated under reduced pressure to give a residue. The crude product was purified by prep-TLC (SiO₂, petroleum ether/ethyl acetate=3/1) to give desired 3-[7-(3,5-difluoro-2-pyridyl)-4,7-diazaspiro[2.5]octan-4-yl]-4,6-difluoro-1-[(5-isopropylsulfonyl-2-thienyl)sulfonyl]indazole (10.2 mg, 15.4 μmol, 19.4% yield, 95.13% purity) as a white solid. ¹H NMR (400 MHz, DMSO-d₆) δ 8.09-8.03 (m, 1H), 7.91-7.87 (m, 1H), 7.73 (br d, J=3.9 Hz, 2H), 7.66-7.60 (m, 1H), 7.44-7.35 (m, 1H), 3.74-3.67 (m, 2H), 3.56-3.39 (m, 3H), 3.29-3.09 (m, 2H), 1.08 (d, J=6.6 Hz, 6H), 0.87-0.81 (m, 2H), 0.63-0.57 (m, 2H). HPLC: 95.13% (220 nm), 94.70% (215 nm), 97.20 (254 nm). MS (ESI): mass calcd. For C₂₅H₂₃F₄N₅O₄S₃ 629.09 m/z found 630.2 [M+H]⁺.

### Compound 41: 4-chloro-6-fluoro-3-[7-(5-fluoro-2-pyridyl)-4, 7-diazaspiro [2.5]octan-4-yl]-1-[(5-isopropylsulfonyl-2-thienyl) sulfonyl]indazole

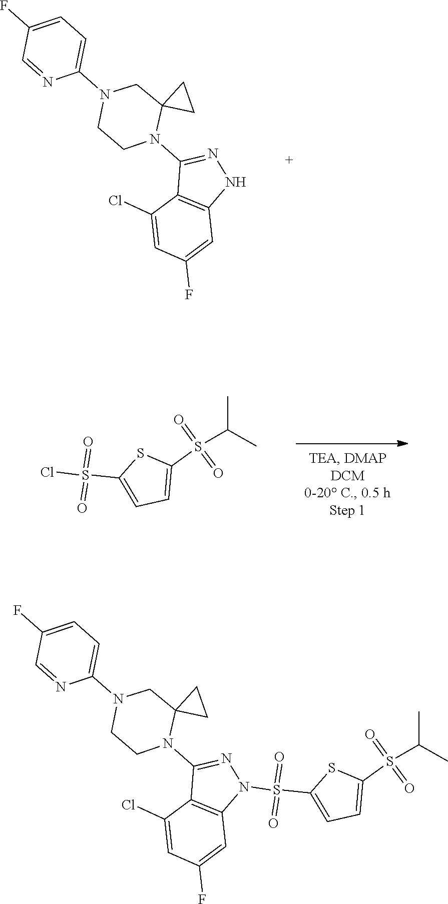 [SMILES: Fc1ccc(N2CCN(c3n[nH]c4cc(F)cc(Cl)c34)C3(CC3)C2)nc1 ; CC(C)S(=O)(=O)c1ccc(S(=O)(=O)Cl)s1 ; CC(C)S(=O)(=O)c1ccc(S(=O)(=O)n2nc(N3CCN(c4ccc(F)cn4)CC34CC4)c3c(Cl)cc(F)cc32)s1]

Step 1: To a solution of 4-chloro-6-fluoro-3-[7-(5-fluoro-2-pyridyl)-4, 7-diazaspiro [2.5]octan-4-yl]-1H-indazole (50.0 mg, 133 μmol, 1 eq) and 5-isopropylsulfonylthiophene-2-sulfonyl chloride (76.9 mg, 266 μmol, 2 eq) in DCM (2 mL) was added TEA (26.9 mg, 266 μmol, 37.0 μL, 2 eq) and DMAP (1.63 mg, 13.3 μmol, 0.1 eq) at 0° C. The mixture was stirred at 20° C. for 0.5 hour. LC-MS showed 4-chloro-6-fluoro-3-[7-(5-fluoro-2-pyridyl)-4, 7-diazaspiro [2.5]octan-4-yl]-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge Prep OBD C18 150*40 mm*10 μm; mobile phase: [water(NH₄HCO₃)-ACN]; B %: 55%-90%, 8 min) to give desired 4-chloro-6-fluoro-3-[7-(5-fluoro-2-pyridyl)-4, 7-diazaspiro [2.5]octan-4-yl]-1-[(5-isopropylsulfonyl-2-thienyl) sulfonyl]indazole (17.7 mg, 28.2 μmol, 21.2% yield, 100.00% purity) as a yellow solid. ¹H NMR (400 MHz, DMSO-d₆) δ 8.09 (d, J=3.0 Hz, 1H), 7.95 (d, J=4.1 Hz, 1H), 7.84-7.75 (m, 2H), 7.61 (dd, J=1.8, 9.1 Hz, 1H), 7.53 (dt, J=3.0, 8.8 Hz, 1H), 6.90 (dd, J=3.3, 9.3 Hz, 1H), 3.67 (br s, 2H), 3.61 (br d, J=4.8 Hz, 2H), 3.59-3.52 (m, 1H), 3.44 (br s, 2H), 1.14 (d, J=6.9 Hz, 6H), 0.82 (s, 2H), 0.50 (br s, 2H). HPLC: 100.00% (220 nm), 99.33% (215 nm), 100.00% (254 nm). MS (ESI): mass calcd. For C₂₅H₂₄ClF₂N₅O₄S₃ 627.06, m/z found 628.1 [M+H]⁺.

### Compound 42: 4-chloro-3-(7-(5-fluoropyridin-2-yl)-4, 7-diazaspiro [2.5]octan-4-yl)-1-((5-(isopropylsulfonyl) thiophen-2-yl) sulfonyl)-1H-indazole

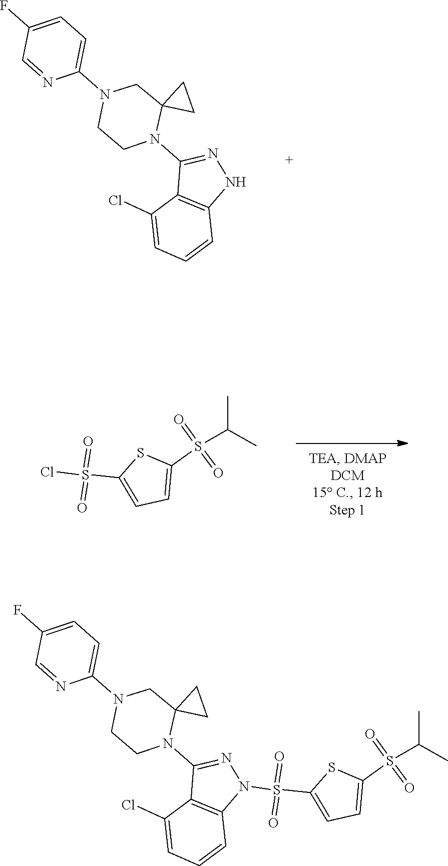 [SMILES: Fc1ccc(N2CCN(c3n[nH]c4cccc(Cl)c34)C3(CC3)C2)nc1 ; CC(C)S(=O)(=O)c1ccc(S(=O)(=O)Cl)s1 ; CC(C)S(=O)(=O)c1ccc(S(=O)(=O)n2nc(N3CCN(c4ccc(F)cn4)CC34CC4)c3c(Cl)cccc32)s1]

Step 1: To a solution of 4-chloro-3-[7-(5-fluoro-2-pyridyl)-4,7-diazaspiro[2.5]octan-4-yl]-1H-indazole (55 mg, 154 μmol, 1 eq) and 5-isopropyl sulfonylthiophene-2-sulfonyl chloride (88.8 mg, 307 μmol, 2 eq) in DCM (2 mL) was added TEA (31.1 mg, 307 μmol, 42.8 μL, 2 eq) and DMAP (1.88 mg, 15.4 μmol, 0.1 eq). The mixture was stirred at 15° C. for 12 hours. LC-MS showed 4-chloro-3-[7-(5-fluoro-2-pyridyl)-4, 7-diazaspiro [2.5]octan-4-yl]-1H-indazole was consumed completely and desired mass was detected. The reaction mixture was concentrated under reduced pressure to give a residue. The residue was purified by prep-HPLC (FA condition, column: Phenomenex Luna 80*30 mm*3 um; mobile phase: [water(FA)-ACN]; B %: 60%-90%, 8 min) to give desired 4-chloro-3-(7-(5-fluoropyridin-2-yl)-4, 7-diazaspiro [2.5]octan-4-yl)-1-((5-(isopropylsulfonyl) thiophen-2-yl) sulfonyl)-1H-indazole (24.4 mg, 39.8 μmol, 25.9% yield, 99.47% purity) as a white solid. ¹H NMR (400 MHz, DMSO-d₆) δ 8.08 (d, J=2.9 Hz, 1H), 8.01 (d, J=8.6 Hz, 1H), 7.81 (d, J=3.9 Hz, 1H), 7.73 (d, J=3.9 Hz, 1H), 7.66 (t, J=8.2 Hz, 1H), 7.56-7.48 (m, 2H), 6.88 (dd, J=3.3, 9.2 Hz, 1H), 3.71-3.58 (m, 4H), 3.57-3.50 (m, 1H), 3.42 (td, J=2.2, 4.7 Hz, 2H), 1.09 (d, J=6.8 Hz, 6H), 0.81 (br s, 2H), 0.47 (br s, 2H). HPLC: 99.47% (220 nm), 99.57% (215 nm), 99.62% (254 nm). MS (ESI): mass calcd. For C₂₅H₂₅ClFN₅O₄S₃ 609.07 m/z found 610.2 [M+H]⁺.

### Compound 43: 4-chloro-3-[7-(5-chloropyrimidin-2-yl)-4, 7-diazaspiro [2.5]octan-4-yl]-1-[(5-isopropylsulfonyl-2-thienyl) sulfonyl]indazole

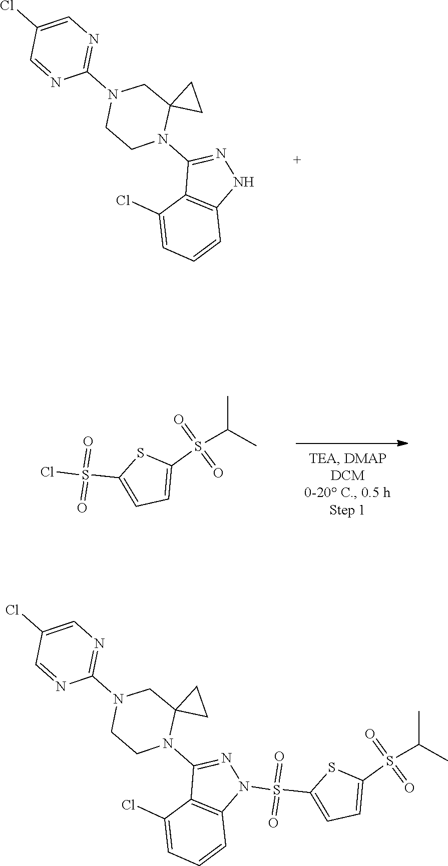 [SMILES: Clc1cnc(N2CCN(c3n[nH]c4cccc(Cl)c34)C3(CC3)C2)nc1 ; CC(C)S(=O)(=O)c1ccc(S(=O)(=O)Cl)s1 ; CC(C)S(=O)(=O)c1ccc(S(=O)(=O)n2nc(N3CCN(c4ncc(Cl)cn4)CC34CC4)c3c(Cl)cccc32)s1]

Step 1: To a solution of 5-isopropylsulfonylthiophene-2-sulfonyl chloride (154 mg, 533 mol, 2 eq) and 4-chloro-3-[7-(5-chloropyrimidin-2-yl)-4,7-diazaspiro[2.5]octan-4-yl]-1H-indazole (100 mg, 267 μmol, 1 eq) in DCM (2 mL) was added TEA (53.9 mg, 533 μmol, 74.2 μL, 2 eq) and DMAP (3.26 mg, 26.7 μmol, 0.1 eq) at 0° C. The mixture was stirred at 20° C. for 0.5 hour. LC-MS showed 5-isopropylsulfonylthiophene-2-sulfonyl chloride was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Phenomenex C18 75*30 mm*3 m; mobile phase: [water(NH₄HCO₃)-ACN]; B %: 60%-90%, 8 min) to give desired 4-chloro-3-[7-(5-chloropyrimidin-2-yl)-4,7-diazaspiro[2.5]octan-4-yl]-1-[(5-isopropylsulfonyl-2-thienyl)sulfonyl]indazole (55.5 mg, 87.9 μmol, 33.0% yield, 99.45% purity) as a white solid. ¹H NMR (DMSO-d₆) δ 8.43 (s, 2H), 8.03 (d, 1H), 7.85 (d, 1H), 7.77 (d, 1H), 7.68 (m, 1H), 7.53 (d, 1H), 3.99 (s, 2H), 3.63-3.81 (m, 2H), 3.52-3.63 (m, 3H), 1.12 (d, 6H), 0.82 (br s, 2H), 0.49 (br s, 2H). HPLC: 99.45% (220 nm), 99.55% (215 nm), 99.64% (254 nm). MS (ESI): mass calcd. For C₂₄H₂₄C₁₂N₆O₄S₃ 626.04, m/z found 627.0 [M+H]⁺.

### Compound 44: 4-chloro-3-[7-(5-fluoropyrimidin-2-yl)-4, 7-diazaspiro [2.5]octan-4-yl]-1-[(5-isopropylsulfonyl-2-thienyl) sulfonyl]indazole

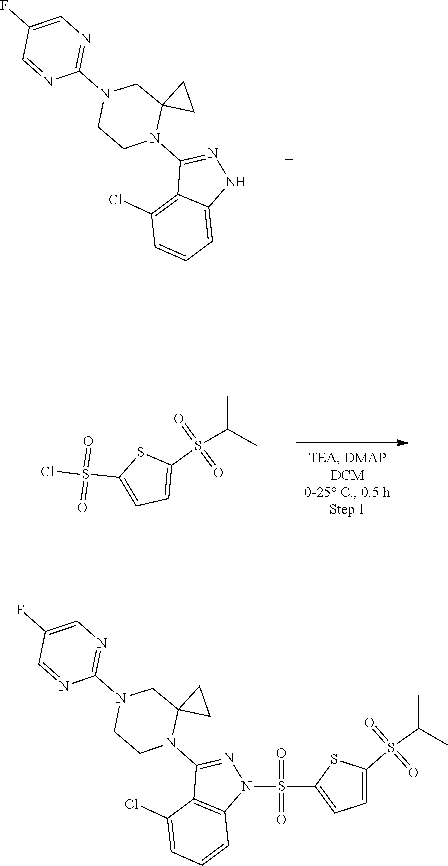 [SMILES: Fc1cnc(N2CCN(c3n[nH]c4cccc(Cl)c34)C3(CC3)C2)nc1 ; CC(C)S(=O)(=O)c1ccc(S(=O)(=O)Cl)s1 ; CC(C)S(=O)(=O)c1ccc(S(=O)(=O)n2nc(N3CCN(c4ncc(F)cn4)CC34CC4)c3c(Cl)cccc32)s1]

Step 1: To a solution of 4-chloro-3-[7-(5-fluoropyrimidin-2-yl)-4,7-diazaspiro[2.5]octan-4-yl]-1H-indazole (100 mg, 279 μmol, 1 eq) and 5-isopropylsulfonylthiophene-2-sulfonyl chloride (80.5 mg, 279 μmol, 1 eq) in DCM (2 mL) was added TEA (56.4 mg, 557 μmol, 77.6 μL, 2 eq) and DMAP (3.40 mg, 27.9 μmol, 0.1 eq) at 0° C. The mixture was stirred at 25° C. for 0.5 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give a residue. The residue was purified by prep-HPLC (FA condition; according to LCMS; Method: column: Phenomenex Luna C18 75*30 mm*3 μm; mobile phase: [water (FA)-ACN]; B %: 50%-80%, 8 min) to give desired 4-chloro-3-[7-(5-fluoropyrimidin-2-yl)-4, 7-diazaspiro [2.5]octan-4-yl]-1-[(5-isopropylsulfonyl-2-thienyl) sulfonyl]indazole (51.5 mg, 83.5 μmol, 30.0% yield, 99.12% purity) as a white solid. ¹H NMR (400 MHz, DMSO-d₆) δ 8.47 (s, 2H), 7.99 (s, 1H), 7.84 (d, J=3.9 Hz, 1H), 7.78-7.75 (m, 1H), 7.72-7.65 (m, 1H), 7.53 (d, J=7.9 Hz, 1H), 4.00-3.92 (m, 2H), 3.73-3.53 (m, 5H), 1.12 (d, J=6.8 Hz, 6H), 0.87-0.76 (m, 2H), 0.48 (br s, 2H). HPLC: 99.12% (220 nm), 98.28% (215 nm), 99.89% (254 nm). MS (ESI): mass calcd. For C₂₄H₂₄ClFN₆O₄S₃ 610.07, m/z found 611.1 [M+H]⁺.

### Compound 45: 4-chloro-3-[7-(3, 5-difluoro-2-pyridyl)-4, 7-diazaspiro [2.5]octan-4-yl]-1-[(5-isopropylsulfonyl-2-thienyl) sulfonyl]indazole

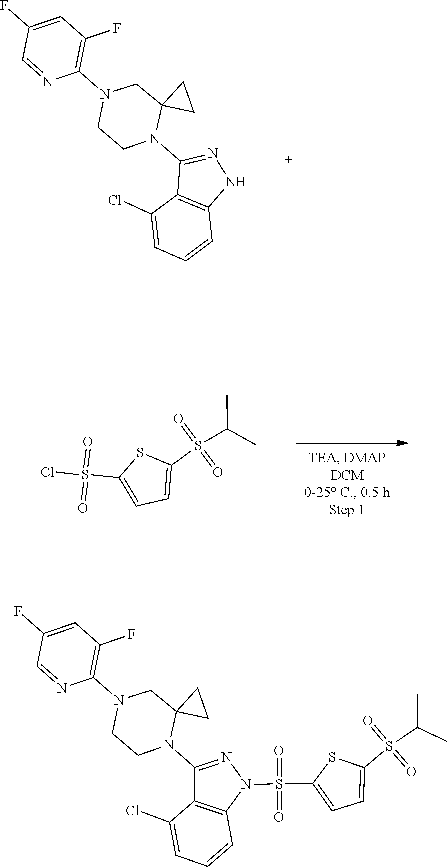 [SMILES: Fc1cnc(N2CCN(c3n[nH]c4cccc(Cl)c34)C3(CC3)C2)c(F)c1 ; CC(C)S(=O)(=O)c1ccc(S(=O)(=O)Cl)s1 ; CC(C)S(=O)(=O)c1ccc(S(=O)(=O)n2nc(N3CCN(c4ncc(F)cc4F)CC34CC4)c3c(Cl)cccc32)s1]

Step 1: To a solution of 4-chloro-3-[7-(3, 5-difluoro-2-pyridyl)-4, 7-diazaspiro [2.5]octan-4-yl]-1H-indazole (100 mg, 266 μmol, 1 eq) and 5-isopropylsulfonylthiophene-2-sulfonyl chloride (154 mg, 532 μmol, 2 eq) in DCM (2 mL) was added TEA (53.9 mg, 532 μmol, 74.1 μL, 2 eq) and DMAP (3.25 mg, 26.6 μmol, 0.1 eq) at 0° C. The mixture was stirred at 25° C. for 0.5 hour. LC-MS showed 4-chloro-3-[7-(3, 5-difluoro-2-pyridyl)-4, 7-diazaspiro [2.5]octan-4-yl]-1H-indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-HPLC (FA condition; according to LCMS; Method: column: Phenomenex Luna C18 75*30 mm*3 μm; mobile phase: [water(FA)-ACN]; B %: 60%-100%, 8 min) to give desired 4-chloro-3-[7-(3, 5-difluoro-2-pyridyl)-4, 7-diazaspiro [2.5]octan-4-yl]-1-[(5-isopropylsulfonyl-2-thienyl) sulfonyl]indazole (32.9 mg, 52.0 μmol, 19.5% yield, 99.24% purity) as a white solid. ¹H NMR (400 MHz, DMSO-d₆) δ 8.11 (d, J=2.2 Hz, 1H), 8.04-8.00 (m, 1H), 7.85-7.78 (m, 2H), 7.77-7.74 (m, 1H), 7.70-7.65 (m, 1H), 7.53 (d, J=7.7 Hz, 1H), 3.71-3.64 (m, 2H), 3.63-3.49 (m, 3H), 3.32-3.21 (m, 2H), 1.11 (d, J=6.8 Hz, 6H), 0.81 (br d, J=1.3 Hz, 2H), 0.50 (br s, 2H). HPLC: 99.19% (220 nm), 99.24% (215 nm), 99.49% (254 nm). MS (ESI): mass calcd. For C₂₅H₂₄ClF₂N₅O₄S₃ 627.06, m/z found 628.1 [M+H]⁺.

### Compound 46: 5-chloro-2-[4-[4-chloro-1-[(5-isopropylsulfonyl-2-thienyl) sulfonyl]indazol-3-yl]-4, 7-diazaspiro [2.5]octan-7-yl]thiazole

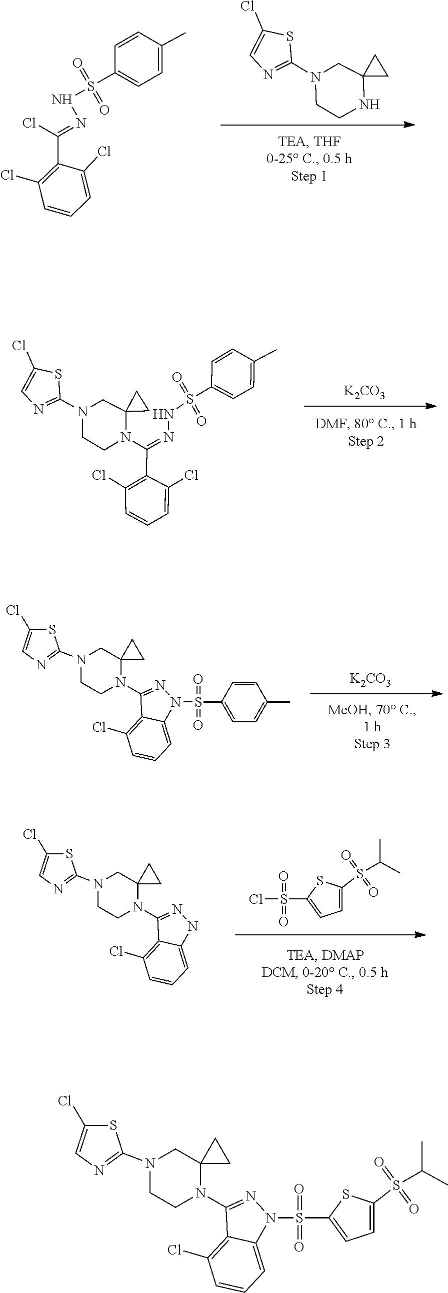

Step 1: To a solution of 5-chloro-2-(4, 7-diazaspiro [2.5]octan-7-yl)thiazole (244 mg, 918 μmol, 1 eq, HCl) in THF (5 mL) was added dropwise TEA (930 mg, 9.18 mmol, 1.28 mL, 10 eq) at 25° C. After addition, the mixture was stirred at this temperature for 10 minutes, and then (1Z)-2,6-dichloro-N-(p-tolylsulfonyl)benzohydrazonoyl chloride (382 mg, 1.01 mmol, 1.1 eq) in THF (2 mL) was added dropwise at 0° C. The resulting mixture was stirred at 25° C. for 20 minutes. LC-MS showed 5-chloro-2-(4, 7-diazaspiro [2.5]octan-7-yl) thiazole was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(Z)—[[7-(5-chlorothiazol-2-yl)-4,7-diazaspiro [2.5]octan-4-yl]-(2,6-dichlorophenyl)methylene]amino]-4-methyl-benzenesulfonamide (520 mg, crude) was obtained as a yellow solid. For C₂₃H₂₂Cl₃N₅O₂S₂ 569.03, m/z found 570.1 [M+H]⁺.

Step 2: To a solution of N—[(Z)—[[7-(5-chlorothiazol-2-yl)-4, 7-diazaspiro [2.5]octan-4-yl]-(2, 6-dichlorophenyl) methylene]amino]-4-methyl-benzenesulfonamide (520 mg, 911 μmol, 1 eq) in DMF (10 mL) was added K₂CO₃ (504 mg, 3.64 mmol, 4 eq). The mixture was stirred at 80° C. for 1 hour. LC-MS showed N—[(Z)—[[7-(5-chlorothiazol-2-yl)-4, 7-diazaspiro [2.5]octan-4-yl]-(2, 6-dichlorophenyl) methylene]amino]-4-methyl-benzenesulfonamide was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give desired 5-chloro-2-[4-[4-chloro-1-(p-tolylsulfonyl)indazol-3-yl]-4, 7-diazaspiro [2.5]octan-7-yl]thiazole (500 mg, crude) as a black oil. For C₂₃H₂₁C₁₂N₅O₂S₂ 533.05, m/z found 534.0 [M+H]⁺.

Step 3: To a solution of 5-chloro-2-[4-[4-chloro-1-(p-tolylsulfonyl) indazol-3-yl]-4, 7-diazaspiro [2.5]octan-7-yl]thiazole (500 mg, 936 μmol, 1 eq) in MeOH (10 mL) was added K₂CO₃ (652 mg, 4.72 mmol, 5 eq). The mixture was stirred at 70° C. for 1 hour. LC-MS showed 5-chloro-2-[4-[4-chloro-1-(p-tolylsulfonyl) indazol-3-yl]-4, 7-diazaspiro [2.5]octan-7-yl]thiazole was consumed completely and desired compound was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by column chromatography (SiO₂, petroleum ether/Ethyl acetate=1/0 to 1/1) to give desired 5-chloro-2-[4-(4-chloro-1H-indazol-3-yl)-4, 7-diazaspiro [2.5]octan-7-yl]thiazole (130 mg, 342 μmol) as a yellow oil. MS (ESI): mass calcd. For C₁₆H₁₅C₁₂N₅S 379.04, m/z found 379.9 [M+H]⁺.

Step 4: To a solution of 5-isopropylsulfonylthiophene-2-sulfonyl chloride (152 mg, 526 mol, 2 eq) and 5-chloro-2-[4-(4-chloro-1H-indazol-3-yl)-4, 7-diazaspiro [2.5]octan-7-yl]thiazole (100 mg, 263 μmol, 1 eq) in DCM (2 mL) was added TEA (53.2 mg, 526 μmol, 73.2 μL, 2 eq) and DMAP (3.21 mg, 26.3 μmol, 0.1 eq) at 0° C. The mixture was stirred at 20° C. for 0.5 hour. LC-MS showed 5-isopropylsulfonylthiophene-2-sulfonyl chloride was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Phenomenex C18 75*30 mm*3 m; mobile phase: [water(NH₄HCO₃)-ACN]; B %: 60%-90%, 8 min) to give desired 5-chloro-2-[4-[4-chloro-1-[(5-isopropylsulfonyl-2-thienyl)sulfonyl]indazol-3-yl]-4,7-diazaspiro[2.5]octan-7-yl]thiazole (31 mg, 48.5 μmol, 18.4% yield, 98.94% purity) as a white solid. ¹H NMR (DMSO-d₆) δ 8.02 (d, 1H), 7.85 (d, 1H), 7.77 (d, 1H), 7.68 (m, 1H), 7.53 (d, 1H), 7.18 (s, 1H), 3.51-3.74 (m, 5H), 3.36 (br s, 2H), 1.13 (d, 6H), 0.88 (br s, 2H), 0.51 (br s, 2H). HPLC: 98.94% (220 nm), 99.02% (215 nm), 97.93% (254 nm). MS (ESI): mass calcd. For C₂₃H₂₃C₁₂N₅O₄S₄ 631.00, m/z found 632.0 [M+H]⁺.

### Compound 47: 4-chloro-3-[7-(5-chloro-2-pyridyl)-4,7-diazaspiro[2.5]octan-4-yl]-1-[(5-isopropylsulfonyl-2-thienyl)sulfonyl]indazole

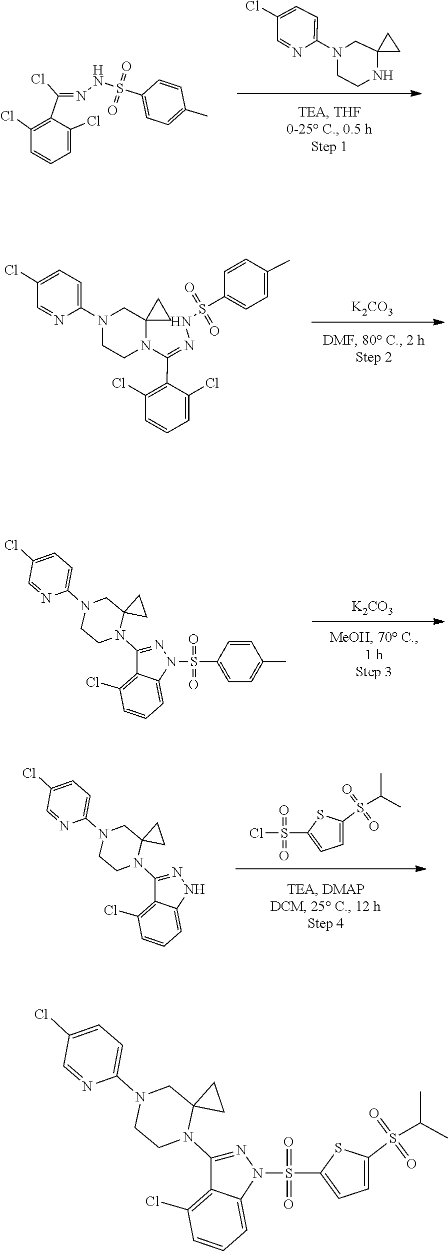

Step 1: To a solution of 7-(5-chloro-2-pyridyl)-4,7-diazaspiro[2.5]octane (320 mg, 1.43 mmol, 1 eq) in THF (3 mL) was added dropwise TEA (1.45 g, 14.3 mmol, 1.99 mL, 10 eq) at 25° C. After addition, the mixture was stirred at this temperature for 10 minutes, and then(1Z)-2,6-dichloro-N-(p-tolylsulfonyl)benzohydrazonoyl chloride (594 mg, 1.57 mmol, 1.1 eq) in THF (1 mL) was added dropwise at 0° C. The resulting mixture was stirred at 25° C. for 20 minutes. LC-MS showed 7-(5-chloro-2-pyridyl)-4, 7-diazaspiro [2.5]octane was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N—[(Z)—[[7-(5-chloro-2-pyridyl)-4, 7-diazaspiro [2.5]octan-4-yl]-(2, 6-dichlorophenyl) methylene]amino]-4-methyl-benzenesulfonamide (980 mg, crude) as a yellow solid. MS (ESI): mass calcd. For C₂₅H₂₄C₁₃N₅O₂S 563.07 m/z found 564.1 [M+H]⁺.

Step 2: To a solution of N—[(Z)—[[7-(5-chloro-2-pyridyl)-4, 7-diazaspiro [2.5]octan-4-yl]-(2, 6-dichlorophenyl) methylene]amino]-4-methyl-benzenesulfonamide (980 mg, 1.73 mmol, 1 eq) in DMF (10 mL) was added K₂CO₃ (1.20 g, 8.67 mmol, 5 eq). The mixture was stirred at 80° C. for 2 hours. LC-MS showed N—[(Z)—[[7-(5-chloro-2-pyridyl)-4, 7-diazaspiro [2.5]octan-4-yl]-(2, 6-dichlorophenyl) methylene]amino]-4-methyl-benzenesulfonamide was consumed completely and desired mass was detected. The reaction mixture was filtered and the filter liquor was concentrated under reduced pressure to give desired 4-chloro-3-[7-(5-chloro-2-pyridyl)-4, 7-diazaspiro [2.5]octan-4-yl]-1-(p-tolylsulfonyl) indazole (980 mg, crude) as a yellow oil. MS (ESI): mass calcd. For C₂₅H₂₃C₁₂N₅O₂S 527.09 m/z found 528.1 [M+H]⁺.

Step 3: To a solution of 4-chloro-3-[7-(5-chloro-2-pyridyl)-4, 7-diazaspiro [2.5]octan-4-yl]-1-(p-tolylsulfonyl) indazole (980 mg, 1.85 mmol, 1 eq) in MeOH (10 mL) was added K₂CO₃ (1.28 g, 9.27 mmol, 5 eq). The mixture was stirred at 70° C. for 1 hour. LC-MS showed 4-chloro-3-[7-(5-chloro-2-pyridyl)-4, 7-diazaspiro [2.5]octan-4-yl]-1-(p-tolylsulfonyl) indazole was consumed completely and desired mass was detected. The reaction mixture was filtered and the filter liquor was concentrated under reduced pressure to give a residue. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by column chromatography (SiO₂, petroleum ether/Ethyl acetate=10/1 to 2/1) to give desired 4-chloro-3-[7-(5-chloro-2-pyridyl)-4, 7-diazaspiro [2.5]octan-4-yl]-1H-indazole (240 mg, 641 μmol, 34.6% yield) as a white oil. MS (ESI): mass calcd. For C₁₈H₁₇C₁₂N₅ 373.09, m/z found 374.1 [M+H]⁺.

Step 4: To a solution of 5-isopropylsulfonylthiophene-2-sulfonyl chloride (77.2 mg, 267 mol, 2 eq) and 4-chloro-3-[7-(5-chloro-2-pyridyl)-4, 7-diazaspiro [2.5]octan-4-yl]-1H-indazole (50 mg, 134 μmol, 1 eq) in DCM (2 mL) was added TEA (27.0 mg, 267 μmol, 37.2 μL, 2 eq) and DMAP (1.63 mg, 13.4 μmol, 0.1 eq). The mixture was stirred at 25° C. for 12 hours. LC-MS showed 4-chloro-3-[7-(5-chloro-2-pyridyl)-4, 7-diazaspiro [2.5]octan-4-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-HPLC (FA condition, according to LCMS; Method: column: Phenomenex Luna C18 75*30 mm*3 μm; mobile phase: [water(FA)-ACN]; B %: 60%-90%, 8 min) to give desired 4-chloro-3-[7-(5-chloro-2-pyridyl)-4, 7-diazaspiro [2.5]octan-4-yl]-1-[(5-isopropylsulfonyl-2-thienyl)sulfonyl]indazole (41.5 mg, 66.1 μmol, 49.5% yield, 99.74% purity) as a yellow solid. ¹H NMR (400 MHz, DMSO-d₆) δ 8.12-8.08 (m, 1H), 8.02 (d, J=8.3 Hz, 1H), 7.85-7.81 (m, 1H), 7.76-7.74 (m, 1H), 7.70-7.65 (m, 1H), 7.61 (dd, J=2.7, 9.0 Hz, 1H), 7.55-7.50 (m, 1H), 6.92-6.88 (m, 1H), 3.78-3.70 (m, 2H), 3.65-3.59 (m, 2H), 3.48 (br s, 3H), 1.11 (d, J=6.7 Hz, 6H), 0.86-0.79 (m, 2H), 0.52-0.45 (m, 2H). HPLC: 99.74% (220 nm), 99.63% (215 nm), 90.62% (254 nm). MS (ESI): mass calcd. For C₂₅H₂₅C₁₂N₅O₄S₃ 625.04 m/z found 626.1 [M+H]⁺.

### Compound 48: 1-[(5-tert-butylsulfonyl-2-thienyl) sulfonyl]-4-chloro-3-[7-(5-fluoro-2-pyridyl)-4, 7-diazaspiro [2.5]octan-4-yl]indazole

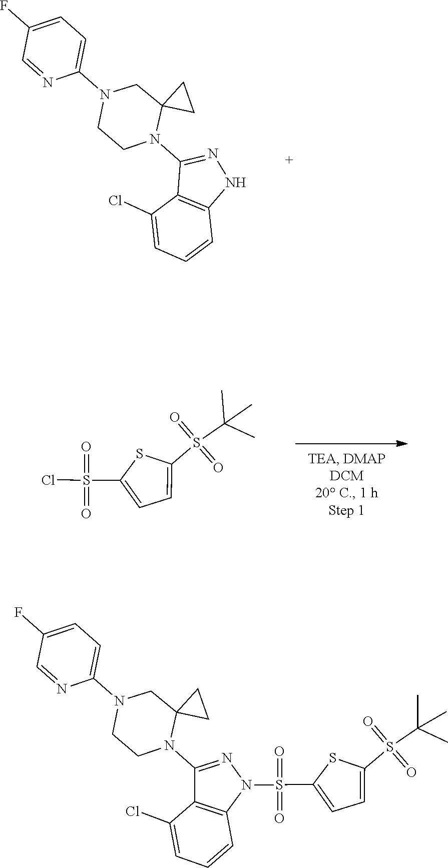 [SMILES: Fc1ccc(N2CCN(c3n[nH]c4cccc(Cl)c34)C3(CC3)C2)nc1 ; CC(C)(C)S(=O)(=O)c1ccc(S(=O)(=O)Cl)s1 ; CC(C)(C)S(=O)(=O)c1ccc(S(=O)(=O)n2nc(N3CCN(c4ccc(F)cn4)CC34CC4)c3c(Cl)cccc32)s1]

Step 1: To the solution of 4-chloro-3-[7-(5-fluoro-2-pyridyl)-4, 7-diazaspiro [2.5]octan-4-yl]-1H-indazole (50.0 mg, 140 μmol, 1 eq), TEA (42.4 mg, 419 μmol, 3 eq) and DMAP (1.71 mg, 14.0 μmol, 0.1 eq) in DCM (2 mL) was added 5-tert-butylsulfonylthiophene-2-sulfonyl chloride (42.3 mg, 140 μmol, 1 eq) at 20° C. and the solution was stirred at 20° C. for 1 hour. LCMS showed 4-chloro-3-[7-(5-fluoro-2-pyridyl)-4, 7-diazaspiro [2.5]octan-4-yl]-1H-indazole was consumed completely and desired mass was detected. The reaction was concentrated to get a residue. The residue was purified by prep-HPLC (column: Phenomenex Luna C18 75*30 mm*3 μm; mobile phase: [water (FA)-ACN]; B %: 35%-80%, 8 min) to give desired 1-[(5-tert-butylsulfonyl-2-thienyl)sulfonyl]-4-chloro-3-[7-(5-fluoro-2-pyridyl)-4,7-diazaspiro[2.5]octan-4-yl]indazole (30.6 mg, 47.5 μmol, 34.0% yield, 96.88% purity) as a light yellow solid. ¹H NMR (400 MHz, DMSO-d₆) δ 8.09 (d, 1H), 8.02 (d, 1H), 7.82 (d, 1H), 7.72 (d, 1H), 7.68 (m, 1H), 7.49-7.56 (m, 2H), 6.90 (m, 1H), 3.68 (s, 2H), 3.63 (br m, 2H), 3.44 (br s, 2H), 1.20 (s, 9H), 0.82 (s, 2H), 0.48 (s, 2H) HPLC: 96.88% (220 nm), 97.30% (215 nm), 100.0% (254 nm). MS (ESI): mass calcd. For C₂₆H₂₇ClFN₅O₄S₃ 623.09, m/z found 624.0 [M+H]⁺.

### Compound 49: 1-((5-(tert-butylsulfonyl) thiophen-2-yl) sulfonyl)-4-chloro-3-(7-(5-fluoropyrimidin-2-yl)-4, 7-diazaspiro [2.5]octan-4-yl)-1H-indazole

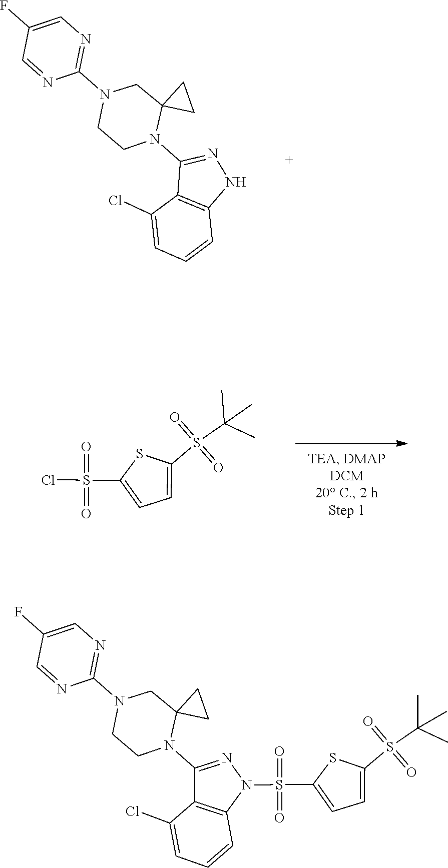 [SMILES: Fc1cnc(N2CCN(c3n[nH]c4cccc(Cl)c34)C3(CC3)C2)nc1 ; CC(C)(C)S(=O)(=O)c1ccc(S(=O)(=O)Cl)s1 ; CC(C)(C)S(=O)(=O)c1ccc(S(=O)(=O)n2nc(N3CCN(c4ncc(F)cn4)CC34CC4)c3c(Cl)cccc32)s1]

Step 1: To the solution of 4-chloro-3-[7-(5-fluoropyrimidin-2-yl)-4,7-diazaspiro[2.5]octan-4-yl]-1H-indazole (50 mg, 140 μmol, 1 eq), TEA (42.3 mg, 418 μmol, 3 eq) and DMAP (1.70 mg, 13.9 μmol, 0.1 eq) in DCM (3 mL) was added 5-tert-butylsulfonylthiophene-2-sulfonyl chloride (42.20 mg, 139 μmol, 1 eq) at 20° C. and the solution was stirred at 20° C. for 2 hours. LCMS showed 4-chloro-3-[7-(5-fluoropyrimidin-2-yl)-4, 7-diazaspiro [2.5]octan-4-yl]-1H-indazole remained and desired mass was detected. The reaction was concentrated to get a residue. The residue was purified by prep-HPLC (column: Phenomenex Luna C18 75*30 mm*3 μm; mobile phase: [water(FA)-ACN]; B %: 40%-90%, 8 min) to give desired 1-[(5-tert-butylsulfonyl-2-thienyl)sulfonyl]-4-chloro-3-[7-(5-fluoropyrimidin-2-yl)-4,7-diazaspiro[2.5]octan-4-yl]indazole (24.8 mg, 38.8 μmol, 27.8% yield, 97.76% purity) as a white solid. ¹H NMR (400 MHz, DMSO-d₆) δ 8.46 (s, 2H), 8.03 (d, 1H), 7.84 (d, 1H), 7.74 (d, 1H), 7.68 (m, 1H), 7.53 (d, 1H), 3.95 (s, 2H), 3.68 (br s, 2H), 3.59 (br s, 2H), 1.20 (s, 9H), 0.82 (br s, 2H), 0.48 (br s, 2H). HPLC: 97.76% (220 nm), 97.90% (215 nm), 99.72% (254 nm). MS (ESI): mass calcd. For C₂₅H₂₆ClFN₆O₄S₃ 624.09, m/z found 625.0 [M+H]⁺.

### Compound 50: 4-chloro-3-(2-cyclopropyl-4-(5-fluoropyrimidin-2-yl)piperazin-1-yl)-1-((4-(isopropylsulfonyl)phenyl)sulfonyl)-1H-indazole

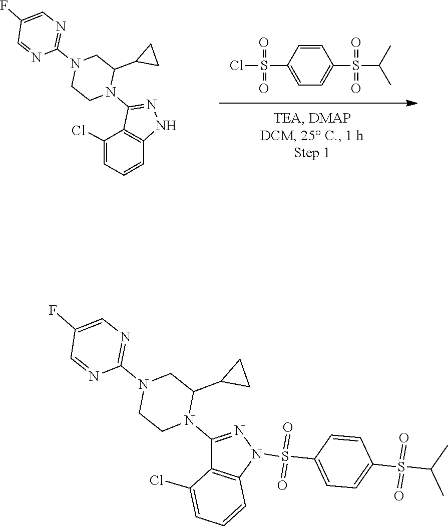 [SMILES: Fc1cnc(N2CCN(c3n[nH]c4cccc(Cl)c34)C(C3CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cn4)CC3C3CC3)c3c(Cl)cccc32)cc1]

Step 1: To a solution of 4-chloro-3-[2-cyclopropyl-4-(5-fluoropyrimidin-2-yl)piperazin-1-yl]-1H-indazole (25 mg, 67.1 μmol, 1 eq) and 4-isopropylsulfonylbenzenesulfonyl chloride (28.4 mg, 101 μmol, 1.5 eq) in DCM (1 mL) was added TEA (13.57 mg, 134.11 umol, 18.67 uL, 2 eq) and DMAP (819 μg, 6.71 umol, 0.1 eq). The mixture was stirred at 25° C. for 1 hour. LC-MS showed 4-chloro-3-[2-cyclopropyl-4-(5-fluoropyrimidin-2-yl)piperazin-1-yl]-1H-indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-HPLC (FA condition; Method: column: Phenomenex C18 75*30 mm*3 μm; mobile phase: [water (FA)-ACN]; B %: 65%-90%, 8 min) to give desired 4-chloro-3-[2-cyclopropyl-4-(5-fluoropyrimidin-2-yl)piperazin-1-yl]-1-(4-isopropylsulfonylphenyl)sulfonyl-indazole (18.1 mg, 28.96 μmol, 43.18% yield, 99.05% purity) as a white solid. ¹H NMR (400 MHz, DMSO-d₆) δ 1H NMR (DMSO-d6) δ: 8.49 (s, 2H), 8.08-8.20 (m, 3H), 8.04 (d, 2H), 7.67 (m, 1H), 7.49 (d, 1H), 4.43 (br d, 2H), 3.57-3.67 (m, 2H), 3.47-3.52 (m, 2H), 3.18-3.27 (m, 1H), 3.05 (br d, 1H), 1.00-1.28 (m, 7H), 0.07-0.23 (m, 1H), −0.10-0.03 (m, 1H), −0.51-−0.41 (m, 1H), −1.06-−0.90 (m, 1H) HPLC: 99.05% (220 nm), 99.09% (215 nm), 99.39% (254 nm). MS (ESI): mass calcd. For C₂₇H₂₈N₆S₂O₄ClF 618.13, m/z found 618.9 [M+H]⁺.

### Compound 51: 4-chloro-3-(2-cyclopropyl-4-(5-fluoropyrimidin-2-yl)piperazin-1-yl)-6-fluoro-1-((4-(isopropylsulfonyl)phenyl)sulfonyl)-1H-indazole

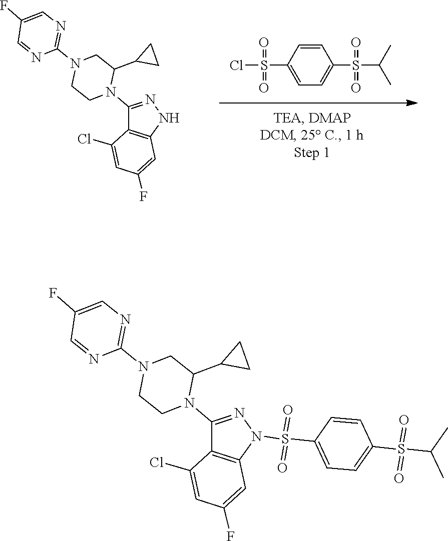 [SMILES: Fc1cnc(N2CCN(c3n[nH]c4cc(F)cc(Cl)c34)C(C3CC3)C2)nc1 ; CC(C)S(=O)(=O)c1ccc(S(=O)(=O)Cl)cc1 ; CC(C)S(=O)(=O)c1ccc(S(=O)(=O)n2nc(N3CCN(c4ncc(F)cn4)CC3C3CC3)c3c(Cl)cc(F)cc32)cc1]

Step 1: To a solution of 4-chloro-3-[2-cyclopropyl-4-(5-fluoropyrimidin-2-yl)piperazin-1-yl]-6-fluoro-1H-indazole (17 mg, 43.5 μmol, 1 eq) and 4-isopropylsulfonylbenzenesulfonyl chloride (18.5 mg, 65.3 μmol, 1.5 eq) in DCM (1 mL) was added TEA (8.80 mg, 87.0 μmol, 2 eq) and DMAP (531 pg, 4.35 μmol, 0.1 eq). The mixture was stirred at 25° C. for 1 hour. LC-MS showed 4-chloro-3-[2-cyclopropyl-4-(5-fluoropyrimidin-2-yl)piperazin-1-yl]-6-fluoro-1H-indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-HPLC (basic condition; method: column: Waters Xbridge Prep OBD C18 150*40 mm*10 μm; mobile phase: [water(NH₄HCO₃)-ACN]; B %: 65%-85%, 8 min) to give desired 4-chloro-3-[2-cyclopropyl-4-(5-fluoropyrimidin-2-yl)piperazin-1-yl]-6-fluoro-1-(4-isopropylsulfonylphenyl)sulfonyl-indazole (6 mg, 8.91 μmol, 20.48% yield, 94.61% purity) as a light yellow solid. ¹H NMR (400 MHz, DMSO-d₆) δ 8.48 (s, 2H), 8.20 (d, J=8.6 Hz, 2H), 8.05 (d, J=8.6 Hz, 2H), 7.89 (dd, J=2.1, 8.8 Hz, 1H), 7.59 (dd, J=2.1, 9.1 Hz, 1H), 4.46-4.37 (m, 2H), 3.64-3.56 (m, 1H), 3.53-3.43 (m, 2H), 3.29-3.19 (m, 3H), 3.03-2.97 (m, 1H), 1.09 (dd, J=2.7, 6.8 Hz, 6H), 0.21 (s, 2H), −0.36-−0.54 (m, 1H), −0.94 (br dd, J=4.7, 9.3 Hz, 1H). HPLC: 94.61% (220 nm), 95.23% (215 nm), 96.03% (254 nm). MS (ESI): mass calcd. For C₂₇H₂₇N₆S₂O₄ClF₂ 636.12, m/z found 636.9 [M+H]⁺.

### Compound 52: 4-chloro-3-(7-(5-fluoropyrimidin-2-yl)-4, 7-diazaspiro [2.5]octan-4-yl)-1-((4-((trifluoromethyl) sulfonyl) phenyl) sulfonyl)-1H-indazole

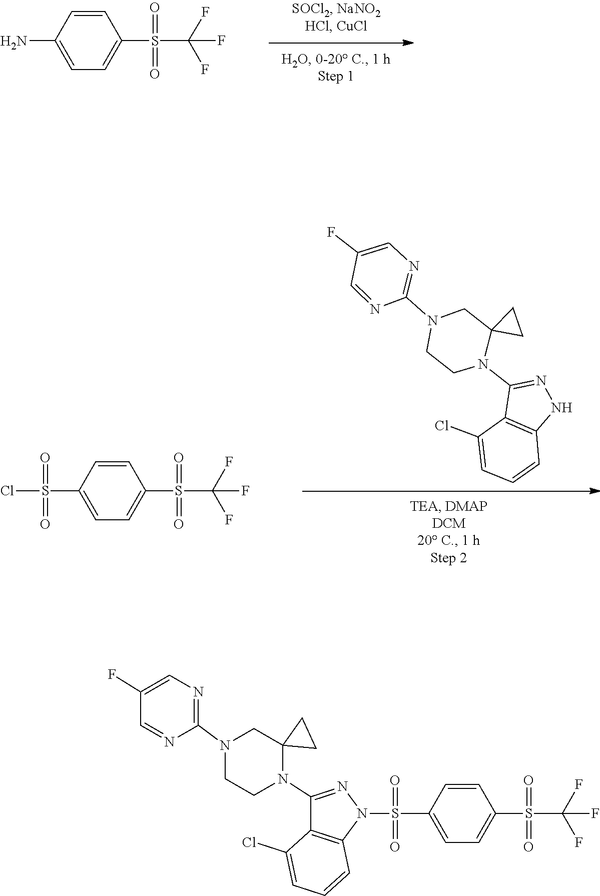 [SMILES: Nc1ccc(S(=O)(=O)C(F)(F)F)cc1 ; O=S(=O)(Cl)c1ccc(S(=O)(=O)C(F)(F)F)cc1 ; Fc1cnc(N2CCN(c3n[nH]c4cccc(Cl)c34)C3(CC3)C2)nc1 ; O=S(=O)(c1ccc(S(=O)(=O)C(F)(F)F)cc1)n1nc(N2CCN(c3ncc(F)cn3)CC23CC3)c2c(Cl)cccc21]

Step 1: To the solution of 4-(trifluoromethylsulfonyl) aniline (1 g, 4.44 mmol, 1 eq) in HCl (10.2 g, 100 mmol, 10 mL, 36% purity, 22.7 eq) was added the solution of NaNO₂ (352 mg, 5.11 mmol, 1.15 eq) in H₂O (10 mL) at 0° C. and the solution was stirred at 0° C. for 0.25 h (Solution A). To H₂O (10 mL) was added SOCl₂ (2.11 g, 17.76 mmol, 1.29 mL, 4 eq) dropwise at 0° C. and the solution was stirred at 20° C. for 0.25 h. To the solution was added CuCl (44.0 mg, 444 μmol, 10.6 uL, 0.1 eq) at 0° C. and the solution was stirred at 0° C. for 0.25 hour. (Solution B). Solution A was added to Solution B at 0° C. and the solution was stirred at 0° C. for 0.25 h. TLC showed 1-benzylsulfanyl-4-(trifluoromethylsulfonyl) benzene remained and a new spot. The reaction was filtered and the cake was washed with water (50 mL). The cake was dried under reduce pressure to give desired 4-(trifluoromethylsulfonyl) benzenesulfonyl chloride (950 mg, crude) as a yellow solid.

Step 2: The solution of 4-chloro-3-[7-(5-fluoropyrimidin-2-yl)-4,7-diazaspiro[2.5]octan-4-yl]-1H-indazole (25 mg, 69.7 μmol, 1 eq), TEA (35.3 mg, 348 μmol, 48.5 μL, 5 eq) and DMAP (851 pg, 6.97 μmol, 0.1 eq) in DCM (2 mL) was added 4-(trifluoromethylsulfonyl)benzenesulfonyl chloride (28.0 mg, 90.6 μmol, 1.3 eq) and the solution was stirred at 20° C. for 1 hour. LC-MS showed 4-chloro-3-[7-(5-fluoropyrimidin-2-yl)-4, 7-diazaspiro [2.5]octan-4-yl]-1H-indazole remained and desired mass was detected. The reaction was concentrated to get a residue. The residue was purified by prep-HPLC (column: Waters Xbridge BEH C18 100*30 mm*10 μm; mobile phase: [water (NH₄HCO₃)-ACN]; B %: 70%-90%, 8 min) to give desired 4-chloro-3-[7-(5-fluoropyrimidin-2-yl)-4,7-diazaspiro[2.5]octan-4-yl]-1-[4-(trifluoromethylsulfonyl)phenyl]sulfonyl-indazole (3 mg, 4.57 μmol, 6.56% yield, 96.20% purity) as a white solid. ¹H NMR (400 MHz, DMSO-d₆) δ 8.46 (s, 2H), 8.31 (d, 2H), 8.18 (d, 2H), 8.09 (d, 1H), 7.68 (m, 1H), 7.51 (d, 1H), 3.90 (s, 2H), 3.48-3.69 (m, 4H), 0.75 (s, 2H), 0.33 (br s, 2H). HPLC: 96.20% (220 nm), 95.41% (215 nm), 98.71% (254 nm). MS (ESI): mass calcd. For C₂₄H₁₉N₆S₂O₄ClF₄ 630.05 mass found 603.9 [M+H]⁺.

### Compound 53: 4-chloro-3-(7, 7-difluoro-4-azaspiro [2.5]octan-4-yl)-1-(4-isopropylsulfonylphenyl) sulfonyl-indazole

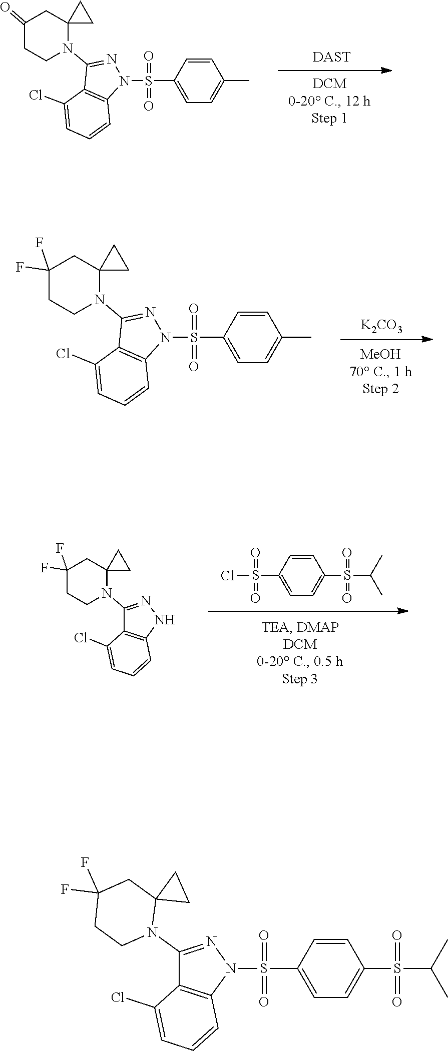

Step 1: To a solution of 4-[4-chloro-1-(p-tolylsulfonyl) indazol-3-yl]-4-azaspiro[2.5]octan-7-one (200 mg, 465 μmol, 1 eq) in DCM (2 mL) was added DAST (150 mg, 930 μmol, 123 μL, 2 eq) at 0° C. The mixture was stirred at 20° C. for 12 hours. LC-MS showed 4-[4-chloro-1-(p-tolylsulfonyl) indazol-3-yl]-4-azaspiro [2.5]octan-7-one was consumed completely and desired mass was detected. The reaction mixture was added MeOH (1 mL) and then was concentrated to give desired 4-chloro-3-(7, 7-difluoro-4-azaspiro [2.5]octan-4-yl)-1-(p-tolylsulfonyl) indazole (210 mg, crude) as yellow oil. MS (ESI): mass calcd. For C₂₁H₂₀ClF₂N₃O₂S 451.09, mass found 452.1 [M+H]⁺.

Step 2: To a solution of 4-chloro-3-(7, 7-difluoro-4-azaspiro [2.5]octan-4-yl)-1-(p-tolylsulfonyl) indazole (210 mg, 465 μmol, 1 eq) in MeOH (4 mL) was added K₂CO₃ (1.28 g, 9.29 mmol, 20 eq). The mixture was stirred at 70° C. for 1 hour. LC-MS showed 4-chloro-3-(7, 7-difluoro-4-azaspiro [2.5]octan-4-yl)-1-(p-tolylsulfonyl) indazole was consumed completely and desired compound was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (Silica gel, petroleum ether/ethyl acetate=2/1) to give desired 4-chloro-3-(7, 7-difluoro-4-azaspiro [2.5]octan-4-yl)-1H-indazole (120 mg, crude) as a yellow oil. MS (ESI): mass calcd. For C₁₄H₁₄ClF₂N₃ 297.08, mass found 298.0 [M+H]⁺.

Step 3: To a solution of 4-isopropylsulfonylbenzenesulfonyl chloride (85.5 mg, 302 μmol, 1.5 eq) and 4-chloro-3-(7, 7-difluoro-4-azaspiro [2.5]octan-4-yl)-1H-indazole (60 mg, 202 μmol, 1 eq) in DCM (2 mL) was added TEA (40.8 mg, 403 μmol, 56.1 μL, 2 eq) and DMAP (2.46 mg, 20.2 μmol, 0.1 eq) at 0° C. The mixture was stirred at 20° C. for 0.5 hour. LC-MS showed 4-chloro-3-(7, 7-difluoro-4-azaspiro [2.5]octan-4-yl)-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Phenomenex C18 75*30 mm*3 m; mobile phase: [water (NH₄HCO₃)-ACN]; B %: 40%-80%, 8 min) to give desired 4-chloro-3-(7, 7-difluoro-4-azaspiro [2.5]octan-4-yl)-1-(4-isopropylsulfonylphenyl) sulfonyl-indazole (25.1 mg, 46.1 μmol, 22.9% yield, 99.88% purity) as a pale yellow solid. ¹H NMR (400 MHz, DMSO-d₆) δ 8.18-7.97 (m, 5H), 7.67 (br t, J=7.3 Hz, 1H), 7.48 (br d, J=7.0 Hz, 1H), 3.66-3.43 (m, 3H), 2.47-2.03 (m, 2H), 1.78 (br s, 2H), 1.11 (br d, J=5.8 Hz, 6H), 0.70 (br s, 2H), 0.37 (br s, 2H). HPLC: 99.88% (220 nm), 99.89% (210 nm), 100.00% (254 nm). MS (ESI): mass calcd. For C₂₃H₂₄ClF₂N₃O₄S₂ 543.09, mass found 543.9 [M+H]⁺.

### Compound 54: 4-chloro-3-(7, 7-dichloro-4-azaspiro [2.5]octan-4-yl)-1-(4-isopropylsulfonylphenyl) sulfonyl-indazole

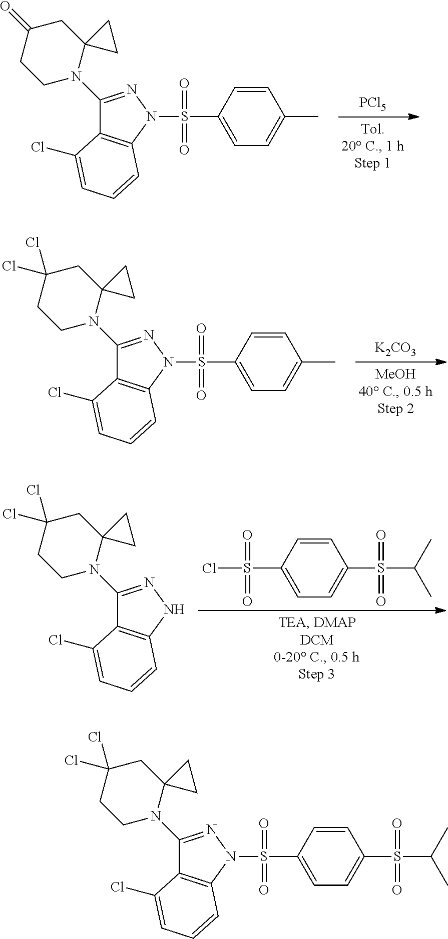

Step 1: To a solution of 4-[4-chloro-1-(p-tolylsulfonyl) indazol-3-yl]-4-azaspiro [2.5]octan-7-one (160 mg, 372 μmol, 1 eq) in Tol. (2 mL) was added PCl₅ (194 mg, 930 μmol, 2.5 eq). The mixture was stirred at 20° C. for 1 hour. LC-MS showed 4-[4-chloro-1-(p-tolylsulfonyl) indazol-3-yl]-4-azaspiro [2.5]octan-7-one was consumed completely and desired mass was detected. The pH value of the mixture was adjusted to 10˜11 with NaOH (2 N). The reaction mixture was added to water (20 mL), extracted with EtOAc (10 mL*3). The combined organic layers were washed with brine 20 mL and dried over Na₂SO₄, filtered and concentrated under reduced pressure to give desired 4-chloro-3-(7,7-dichloro-4-azaspiro[2.5]octan-4-yl)-1-(p-tolylsulfonyl) indazole (220 mg, crude) as a yellow solid. MS (ESI): mass calcd. For C₂₁H₂₀Cl₃N₃O₂S 483.03, mass found 483.9 [M+H]⁺.

Step 2: To a solution of 4-chloro-3-(7, 7-dichloro-4-azaspiro [2.5]octan-4-yl)-1-(p-tolylsulfonyl) indazole (220 mg, 454 μmol, 1 eq) in MeOH (2 mL) was added K₂CO₃ (314 mg, 2.27 mmol, 5 eq). The mixture was stirred at 40° C. for 0.5 hour. TLC (petroleum ether/ethyl acetate=5/1) indicated 4-chloro-3-(7, 7-dichloro-4-azaspiro [2.5]octan-4-yl)-1-(p-tolylsulfonyl) indazole was consumed completely and one new spot was formed. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (Silica gel, petroleum ether/ethyl acetate=5/1) to give desired 4-chloro-3-(7, 7-dichloro-4-azaspiro [2.5]octan-4-yl)-1H-indazole (65 mg, crude) as a yellow solid. MS (ESI): mass calcd. For C₁₄H₁₄Cl₃N₃ 329.03, mass found 330.0 [M+H]⁺.

Step 3: To a solution of 4-isopropylsulfonylbenzenesulfonyl chloride (38.5 mg, 136 μmol, 1.5 eq) and 4-chloro-3-(7, 7-dichloro-4-azaspiro [2.5]octan-4-yl)-1H-indazole (30 mg, 90.7 μmol, 1 eq) in DCM (1 mL) was added TEA (18.4 mg, 181 μmol, 25.3 μL, 2 eq) and DMAP (1.11 mg, 9.07 μmol, 0.1 eq) at 0° C. The mixture was stirred at 20° C. for 0.5 hour. LC-MS showed 4-chloro-3-(7, 7-dichloro-4-azaspiro [2.5]octan-4-yl)-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge Prep OBD C18 150*40 mm*10 μm; mobile phase: [water (NH₄HCO₃)-ACN]; B %: 50%-80%, 8 min) to give desired 4-chloro-3-(7, 7-dichloro-4-azaspiro [2.5]octan-4-yl)-1-(4-isopropylsulfonylphenyl) sulfonyl-indazole (12 mg, 20.8 μmol, 22.9% yield, 100.0% purity) as a yellow solid. ¹H NMR (400 MHz, DMSO-d₆) δ 7.91 (br d, J=8.3 Hz, 1H), 7.86 (s, 4H), 7.48 (br t, J=8.0 Hz, 1H), 7.29 (br d, J=7.5 Hz, 1H), 3.45 (br s, 2H), 3.29 (br dd, J=7.0, 13.8 Hz, 2H), 2.14-1.65 (m, 3H), 0.92 (br d, J=6.6 Hz, 6H), 0.71-0.54 (m, 2H), 0.24 (br s, 2H). HPLC: 100.00% (220 nm), 100.00% (210 nm), 100.00% (254 nm). MS (ESI): mass calcd. For C₂₃H₂₄Cl₃N₃O₄S₂ 575.03, mass found 576.0 [M+H]⁺.

### Compound 55: 1-[4-[4-chloro-1-(4-isopropylsulfonylphenyl) sulfonyl-indazol-3-yl]-4, 7-diazaspiro [2.5]octan-7-yl]-2, 2, 2-trifluoro-ethanone

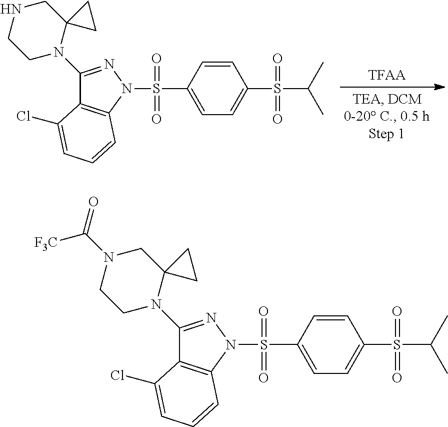

Step 1: To a solution of 4-chloro-3-(4, 7-diazaspiro [2.5]octan-4-yl)-1-(4-isopropylsulfonylphenyl) sulfonyl-indazole (50 mg, 98.2 μmol, 1 eq) in DCM (2 mL) was added TEA (99.4 mg, 982 μmol, 137 μL, 10 eq) and (2,2,2-trifluoroacetyl) 2,2,2-trifluoroacetate(206 mg, 982 μmol, 137 μL, 10 eq) at 0° C. The mixture was stirred at 20° C. for 0.5 hour. LC-MS showed 4-chloro-3-(4, 7-diazaspiro [2.5]octan-4-yl)-1-(4-isopropylsulfonylphenyl) sulfonyl-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Phenomenex C18 75*30 mm*3 μm; mobile phase: [water (NH₄HCO₃)-ACN]; B %: 40%-80%, 8 min) to give desired 1-[4-[4-chloro-1-(4-isopropylsulfonylphenyl) sulfonyl-indazol-3-yl]-4, 7-diazaspiro [2.5]octan-7-yl]-2, 2, 2-trifluoro-ethanone (13.1 mg, 21.6 μmol, 22.0% yield, 99.71% purity) as a pale yellow solid. ¹H NMR (400 MHz, DMSO-d₆) δ 8.15-8.03 (m, 5H), 7.67 (t, J=8.1 Hz, 1H), 7.49 (d, J=7.8 Hz, 1H), 3.84 (s, 1H), 3.82-3.74 (m, 1H), 3.57 (br s, 2H), 3.53-3.44 (m, 2H), 3.33 (s, 1H), 1.10 (d, J=6.8 Hz, 6H), 0.83 (br d, J=14.1 Hz, 2H), 0.39 (br s, 2H). HPLC: 99.71% (220 nm), 99.72% (210 nm), 100.00% (254 nm). MS (ESI): mass calcd. For C₂₄H₂₄ClF₃N₄O₅S₂ 604.08, mass found 604.9 [M+H]⁺.

### Compound 56: 1-[4-[4-chloro-1-(4-isopropylsulfonylphenyl)sulfonyl-indazol-3-yl]-4,7-diazaspiro[2.5]octan-7-yl]ethanone

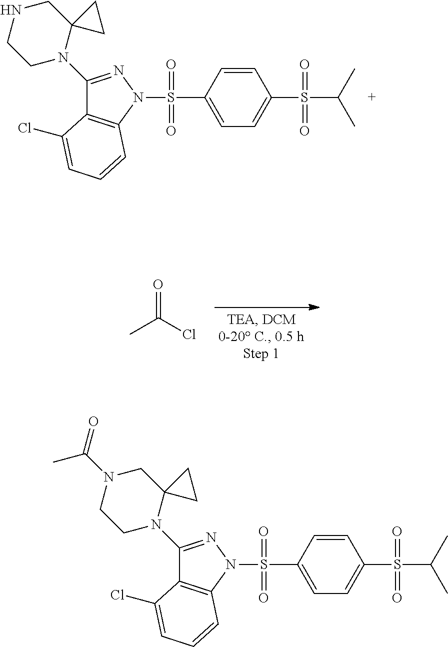

Step 1: To a solution of 4-chloro-3-(4, 7-diazaspiro [2.5]octan-4-yl)-1-(4-isopropylsulfonylphenyl) sulfonyl-indazole (30 mg, 58.9 μmol, 1 eq) in DCM (2 mL) was added TEA (59.6 mg, 589 μmol, 82.0 μL, 10 eq) and acetyl chloride (23.1 mg, 295 μmol, 21.0 μL, 5 eq) at 0° C. The mixture was stirred at 20° C. for 0.5 hour. LC-MS showed 4-chloro-3-(4, 7-diazaspiro [2.5]octan-4-yl)-1-(4-isopropylsulfonylphenyl) sulfonyl-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water(NH₄HCO₃)-ACN]; B %: 45%-85%, 8 min) to give desired 1-[4-[4-chloro-1-(4-isopropylsulfonylphenyl) sulfonyl-indazol-3-yl]-4, 7-diazaspiro [2.5]octan-7-yl]ethanone (8.1 mg, 14.68 μmol, 24.92% yield, 99.90% purity) as a white solid. ¹H NMR (400 MHz, DMSO-d₆) δ 8.12-8.01 (m, 5H), 7.66 (t, J=8.1 Hz, 1H), 7.48 (br d, J=7.8 Hz, 1H), 3.65 (s, 2H), 3.53-3.42 (m, 5H), 2.08-1.98 (m, 3H), 1.09 (br d, J=6.6 Hz, 6H), 0.87-0.65 (m, 2H), 0.32 (br d, J=17.9 Hz, 2H). HPLC: 99.90% (220 nm), 99.88% (210 nm), 100.00% (254 nm). MS (ESI): mass calcd. For C₂₄H₂₇ClN₄O₅S₂ 550.11, mass found 551.1 [M+H]⁺.

### Compound 57: [4-[4-chloro-1-(4-isopropylsulfonylphenyl)sulfonyl-indazol-3-yl]-4,7-diazaspiro[2.5]octan-7-yl]-cyclopropyl-methanone

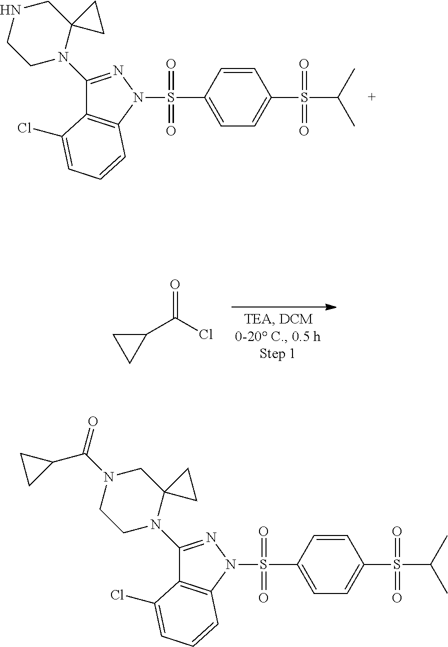 [SMILES: CC(C)S(=O)(=O)c1ccc(S(=O)(=O)n2nc(N3CCNCC34CC4)c3c(Cl)cccc32)cc1 ; O=C(Cl)C1CC1 ; CC(C)S(=O)(=O)c1ccc(S(=O)(=O)n2nc(N3CCN(C(=O)C4CC4)CC34CC4)c3c(Cl)cccc32)cc1]

Step 1: To a solution of 4-chloro-3-(4,7-diazaspiro[2.5]octan-4-yl)-1-(4-isopropylsulfonylphenyl)sulfonyl-indazole (30 mg, 58.9 μmol, 1 eq) in DCM (2 mL) was added TEA (59.6 mg, 589 μmol, 82.0 μL, 10 eq) and cyclopropanecarbonyl chloride (30.8 mg, 295 μmol, 26.8 μL, 5 eq) at 0° C. The mixture was stirred at 20° C. for 0.5 hour. LC-MS showed 4-chloro-3-(4, 7-diazaspiro [2.5]octan-4-yl)-1-(4-isopropylsulfonylphenyl) sulfonyl-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water (NH₄HCO₃)-ACN]; B %: 50%-90%, 8 min) to give desired [4-[4-chloro-1-(4-isopropylsulfonylphenyl) sulfonyl-indazol-3-yl]-4, 7-diazaspiro [2.5]octan-7-yl]-cyclopropyl-methanone (14.2 mg, 24.5 μmol, 41.6% yield, 99.70% purity) as a white solid. ¹H NMR (400 MHz, DMSO-d₆) δ 8.12-8.01 (m, 5H), 7.66 (t, J=8.1 Hz, 1H), 7.48 (d, J=7.8 Hz, 1H), 3.90 (br s, 1H), 3.68 (br s, 1H), 3.63-3.54 (m, 1H), 3.48 (td, J=6.7, 13.6 Hz, 2H), 3.40 (br d, J=2.9 Hz, 2H), 1.96 (br s, 1H), 1.09 (d, J=6.8 Hz, 6H), 0.85-0.65 (m, 6H), 0.42-0.23 (m, 2H). HPLC: 99.70% (220 nm), 99.63% (210 nm), 99.20% (254 nm). MS (ESI): mass calcd. For C₂₆H₂₉ClN₄O₅S₂ 576.13, mass found 577.1 [M+H]⁺.

### Compound 58: [4-[4-chloro-3-[7-(5-fluoropyrimidin-2-yl)-4, 7-diazaspiro [2.5]octan-4-yl]indazol-1-yl]sulfonylphenyl]-imino-isopropyl-oxo-λ6-sulfane

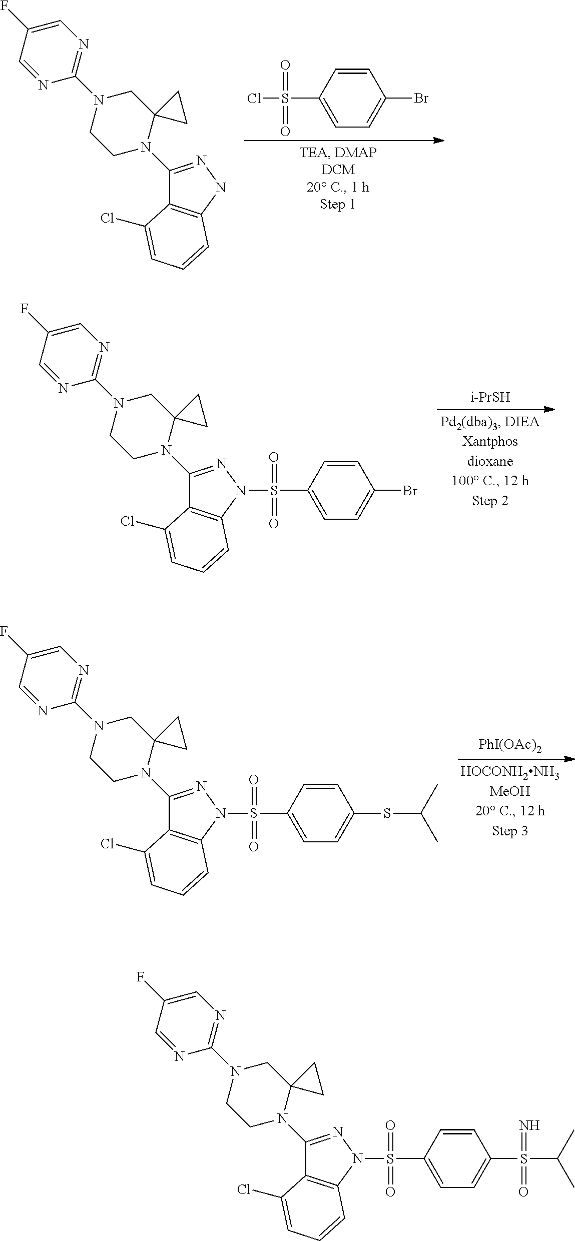

Step 1: The solution of 4-chloro-3-[7-(5-fluoropyrimidin-2-yl)-4,7-diazaspiro[2.5]octan-4-yl]-1H-indazole (50 mg, 139 μmol, 1 eq), TEA (70.5 mg, 697 μmol, 5 eq) and DMAP (1.70 mg, 13.9 μmol, 0.1 eq) in DCM (2 mL) was added 4-bromobenzenesulfonyl chloride (46.3 mg, 181 μmol, 1.3 eq) and the solution was stirred at 20° C. for 1 hour. LCMS showed 4-chloro-3-[7-(5-fluoropyrimidin-2-yl)-4, 7-diazaspiro [2.5]octan-4-yl]-1H-indazole was consumed completely and desired mass was detected. The reaction mixture was concentrated to get a residue. The residue was purified by prep-TLC (Silica gel, petroleum ether/ethyl acetate=2/1) to give desired 1-(4-bromophenyl) sulfonyl-4-chloro-3-[7-(5-fluoropyrimidin-2-yl)-4, 7-diazaspiro [2.5]octan-4-yl]indazole (77.6 mg, 134 μmol, 96.4% yield) as a colorless oil. MS (ESI): mass calcd. For C₂₃H₁₉N₆BrSO₂ClF 576.01 mass found 577.1 [M+H]⁺.

Step 2: The solution of 1-(4-bromophenyl)sulfonyl-4-chloro-3-[7-(5-fluoropyrimidin-2-yl)-4,7-diazaspiro[2.5]octan-4-yl]indazole (57 mg, 98.6 μmol, 1 eq), Xantphos (11.4 mg, 19.7 μmol, 0.2 eq), Pd₂(dba)₃ (9.03 mg, 9.86 μmol, 0.1 eq), DIEA (25.5 mg, 197 μmol, 2 eq) and propane-2-thiol (37.6 mg, 493 μmol, 5 eq) in dioxane (5 mL) was stirred at 100° C. for 12 hours. LCMS showed 1-(4-bromophenyl) sulfonyl-4-chloro-3-[7-(5-fluoropyrimidin-2-yl)-4, 7-diazaspiro [2.5]octan-4-yl]indazole was consumed completely and desired mass was detected. The reaction was concentrated to get a residue. The residue was purified by prep-TLC (Silica gel, petroleum ether/ethyl acetate=2/1) to give desired 4-chloro-3-[7-(5-fluoropyrimidin-2-yl)-4, 7-diazaspiro [2.5]octan-4-yl]-1-(4-isopropylsulfanylphenyl) sulfonyl-indazole (60 mg, crude) was a yellow oil. MS (ESI): mass calcd. For C₂₆H₂₆N₆S₂O₂ClF 572.12 mass found 573.2 [M+H]⁺.

Step 3: To the solution of 4-chloro-3-[7-(5-fluoropyrimidin-2-yl)-4,7-diazaspiro[2.5]octan-4-yl]-1-(4-isopropylsulfanylphenyl)sulfonyl-indazole (60 mg, 105 μmol, 1 eq) in EtOH (2 mL) was added ammonium carbamate (32.7 mg, 419 μmol, 4 eq) and the solution was stirred at 20° C. for 0.5 hour. To the solution was added PhI(OAc)₂ (101 mg, 314 μmol, 3 eq) and the solution was stirred at 20° C. for 11.5 hours. LCMS showed 4-chloro-3-[7-(5-fluoropyrimidin-2-yl)-4, 7-diazaspiro [2.5]octan-4-yl]-1-(4-isopropylsulfanylphenyl) sulfonyl-indazole remained and desired mass was detected. The reaction was concentrated to get a residue. The residue was purified by prep-HPLC (column: Phenomenex Luna 80*30 mm*3 μm; mobile phase: [water (FA)-ACN]; B %: 45%-70%, 8 min) to give desired [4-[4-chloro-3-[7-(5-fluoropyrimidin-2-yl)-4,7-diazaspiro[2.5]octan-4-yl]indazol-1-yl]sulfonylphenyl]-imino-isopropyl-oxo-λ6-sulfane (17.6 mg, 28.4 μmol, 27.09% yield, 97.35% purity) as a white solid. ¹H NMR (DMSO-d₆) δ 8.46 (s, 2H), 8.08 (d, 1H), 7.93-8.05 (m, 4H), 7.65 (m, 1H), 7.47 (d, 1H), 4.45 (s, 1H), 3.93 (s, 2H), 3.51-3.64 (m, 4H), 3.18-3.25 (m, 1H), 1.03 (d, 6H), 0.78 (br s, 2H), 0.28-0.45 (m, 2H). HPLC: 97.35% (220 nm), 98.01% (215 nm), 99.75% (254 nm). MS (ESI): mass calcd. For C₂₆H₂₇N₇S₂O₃ClF 603.13 mass found 604.1 [M+H]⁺.

### Compound 59: 4-chloro-6-fluoro-1-(1-isopropylsulfonylpyrrol-3-yl)sulfonyl-3-[7-(2,2,2-trifluoroethyl)-4,7-diazaspiro[2.5]octan-4-yl]indazole

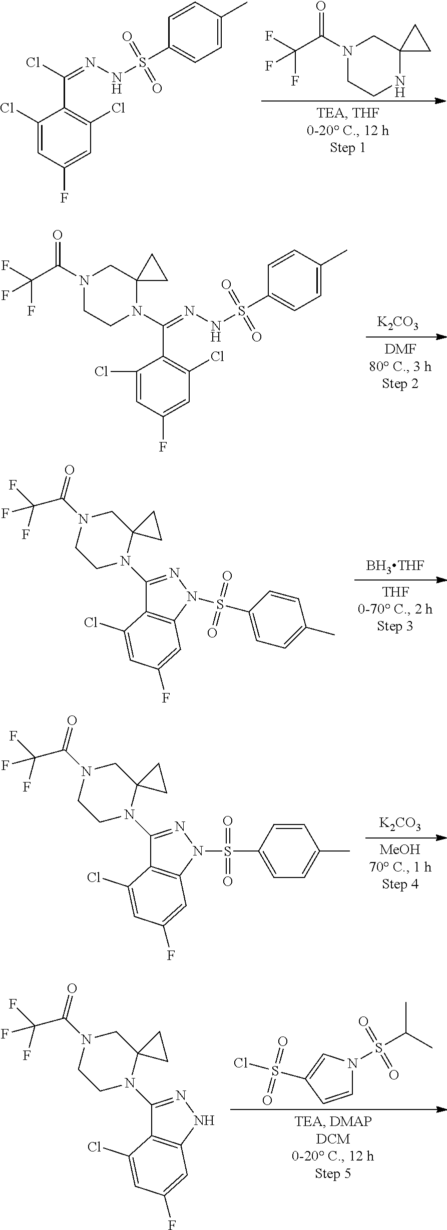

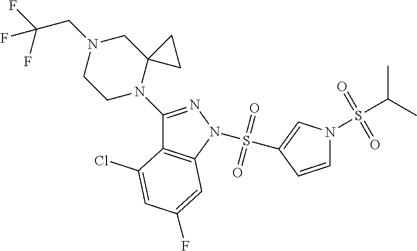 [SMILES: CC(C)S(=O)(=O)n1ccc(S(=O)(=O)n2nc(N3CCN(CC(F)(F)F)CC34CC4)c3c(Cl)cc(F)cc32)c1]

Step 1: To a solution of 1-(4, 7-diazaspiro [2.5]octan-7-yl)-2, 2, 2-trifluoro-ethanone (494 mg, 2.02 mmol, 1 eq, HCl) in THF (7 mL) was added TEA (1.02 g, 10.1 mmol, 1.41 mL, 5 eq). The mixture was stirred at 0° C. for 10 minutes. Then (1E)-2,6-dichloro-4-fluoro-N-(p-tolylsulfonyl) benzohydrazonoyl chloride (800 mg, 2.02 mmol, 1 eq) in THF (10 mL) as added to the mixture. The mixture was stirred at 20° C. for 12 hours. LC-MS showed (1E)-2, 6-dichloro-4-fluoro-N-(p-tolylsulfonyl) benzohydrazonoyl chloride was consumed completely and one main peak with desired mass was detected. The reaction mixture was diluted with H₂O (50 mL) and extracted with EtOAc (20 mL*2). The combined organic layers were washed with brine (20 mL*2), dried over anhydrous Na₂SO₄, filtered and concentrated under reduced pressure to give desired N-[(E)-[(2, 6-dichloro-4-fluoro-phenyl)-[7-(2, 2, 2-trifluoroacetyl)-4, 7-diazaspiro [2.5]octan-4-yl]methylene]amino]-4-methyl-benzenesulfonamide (1.15 g, crude) as a yellow solid. MS (ESI): mass calcd. For C₂₂H₂₀N₄F₄SO₃Cl₂ 566.06 mass found 567.1 [M+H]⁺.

Step 2: To a solution of N-[(E)-[(2, 6-dichloro-4-fluoro-phenyl)-[7-(2, 2, 2-trifluoroacetyl)-4, 7-diazaspiro [2.5]octan-4-yl]methylene]amino]-4-methyl-benzenesulfonamide (1.15 g, 2.03 mmol, 1 eq) in DMF (10 mL) was added K₂CO₃ (2.80 g, 20.3 mmol, 10 eq). The mixture was stirred at 80° C. for 3 hours. LC-MS showed N-[(E)-[(2, 6-dichloro-4-fluoro-phenyl)-[7-(2, 2, 2-trifluoroacetyl)-4, 7-diazaspiro [2.5]octan-4-yl]methylene]amino]-4-methyl-benzenesulfonamide was consumed completely and one main peak with desired mass was detected. The reaction mixture was diluted with H₂O (20 mL) and extracted with MTBE (50 mL*2). The combined organic layers were washed with brine (10 mL*2), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜20% Ethyl acetate/petroleum ether gradient @50 mL/min) to give desired 1-[4-[4-chloro-6-fluoro-1-(p-tolylsulfonyl) indazol-3-yl]-4, 7-diazaspiro [2.5]octan-7-yl]-2, 2, 2-trifluoro-ethanone (500 mg, 941 μmol, 46.5% yield) as a yellow oil. MS (ESI): mass calcd. For C₂₂H₁₉N₄ClF₄SO₃ 530.08 mass found 531.1 [M+H]⁺.

Step 3: To a solution of 1-[4-[4-chloro-6-fluoro-1-(p-tolylsulfonyl) indazol-3-yl]-4,7-diazaspiro[2.5]octan-7-yl]-2,2,2-trifluoro-ethanone (200 mg, 376 μmol, 1 eq) in THF (5 mL) was added BH₃·THF (1 M, 5 mL, 13.3 eq) dropwise at 0° C. The mixture was stirred at 70° C. for 2 hours. LC-MS showed 1-[4-[4-chloro-6-fluoro-1-(p-tolylsulfonyl) indazol-3-yl]-4, 7-diazaspiro [2.5]octan-7-yl]-2, 2, 2-trifluoro-ethanone was consumed completely and one main peak with desired mass was detected. The reaction mixture was quenched by addition MeOH 10 mL at 0° C., and stirred at 0° C. for 30 min, and then concentrated under reduced pressure to give desired 4-chloro-6-fluoro-1-(p-tolylsulfonyl)-3-[7-(2, 2, 2-trifluoroethyl)-4, 7-diazaspiro [2.5]octan-4-yl]indazole (200 mg, crude) as a white solid. MS (ESI): mass calcd. For C₂₂H₂₁N₄ClF₄SO₂ 516.1, mass found 517.2 [M+H]⁺.

Step 4: To a solution of 4-chloro-6-fluoro-1-(p-tolylsulfonyl)-3-[7-(2, 2, 2-trifluoroethyl)-4, 7-diazaspiro [2.5]octan-4-yl]indazole (200 mg, 386 μmol, 1 eq) in MeOH (10 mL) was added K₂CO₃ (267 mg, 1.93 mmol, 5 eq). The mixture was stirred at 70° C. for 1 hour. LC-MS showed 4-chloro-6-fluoro-1-(p-tolylsulfonyl)-3-[7-(2, 2, 2-trifluoroethyl)-4, 7-diazaspiro [2.5]octan-4-yl]indazole was consumed completely and one main peak with desired mass was detected. The reaction mixture was concentrated under reduced pressure to remove MeOH and then it was diluted with H₂O (20 mL) and extracted with EtOAc (10 mL*3). The combined organic layers were washed with brine (10 mL*1), dried over Na₂SO₄, filtered and concentrated under reduced pressure to give a residue. The residue was purified by prep-TLC (Silica gel, petroleum ether/ethyl acetate=2/1) to give desired 4-chloro-6-fluoro-3-[7-(2, 2, 2-trifluoroethyl)-4, 7-diazaspiro [2.5]octan-4-yl]-1H-indazole (50 mg, crude) as a white solid. MS (ESI): mass calcd. For C₁₅H₁₅N₄ClF₄ 362.09, mass found 363.1 [M+H]⁺.

Step 5: To a solution of 4-chloro-6-fluoro-3-[7-(2,2,2-trifluoroethyl)-4,7-diazaspiro[2.5]octan-4-yl]-1H-indazole (50 mg, 137 μmol, 1 eq) and 1-isopropylsulfonylpyrrole-3-sulfonyl chloride (56.1 mg, 206 μmol, 1.5 eq) in DCM (10.0 mL) was added TEA (27.9 mg, 275 μmol, 38.3 μL, 2 eq) and DMAP (1.68 mg, 13.7 μmol, 0.1 eq) at 0° C. The mixture was stirred at 20° C. for 12 hours. LC-MS showed 4-chloro-6-fluoro-3-[7-(2, 2, 2-trifluoroethyl)-4, 7-diazaspiro [2.5]octan-4-yl]-1H-indazole was consumed completely and one main peak with desired mass was detected. The reaction mixture was add to H₂O (20 mL) and extracted with EtOAc (10 mL*3). The combined organic layers were washed with brine (20 mL), dried over Na₂SO₄, filtered and concentrated under reduced pressure to give a residue. The residue was purified by prep-HPLC (neutral condition: column: Welch Xtimate C18 180*70 mm*10 m; mobile phase: [water (NH₄HCO₃)-ACN]; B %: 51%-80%, 15 min) to give desired 4-chloro-6-fluoro-1-(1-isopropylsulfonylpyrrol-3-yl)sulfonyl-3-[7-(2,2,2-trifluoroethyl)-4,7-diazaspiro[2.5]octan-4-yl]indazole (60 mg, 100 μmol, 72.8% yield, 99.61% purity) as a white solid. ¹H NMR (400 MHz, DMSO-d₆) δ 12.85-12.83 (m, 1H), 8.01 (s, 1H), 7.84-7.71 (m, 1H), 7.50 (br d, J=8.3 Hz, 1H), 7.37 (br s, 1H), 6.57 (br s, 1H), 3.94 (td, J=6.7, 13.4 Hz, 1H), 3.46 (br s, 3H), 3.25-3.14 (m, 1H), 3.19 (q, J=10.0 Hz, 1H), 3.28-3.11 (m, 1H), 2.57 (br s, 2H), 1.08 (br d, J=6.8 Hz, 6H), 0.70 (br s, 2H), 0.46 (br s, 2H). HPLC: 99.43% (220 nm), 99.37% (215 nm), 99.61% (254 nm). MS (ESI): mass calcd. For C₂₂H₂₄N₅S₂O₄ClF₄ 597.09 mass found 597.9 [M+H]⁺.

### Compound 60: (S)-4-chloro-3-(2-cyclopropyl-4-(5-fluoropyrimidin-2-yl) piperazin-1-yl)-1-((4-(isopropylsulfonyl) phenyl) sulfonyl)-1H-indazole

### Compound 61: (R)-4-chloro-3-(2-cyclopropyl-4-(5-fluoropyrimidin-2-yl) piperazin-1-yl)-1-((4-(isopropylsulfonyl) phenyl) sulfonyl)-1H-indazole

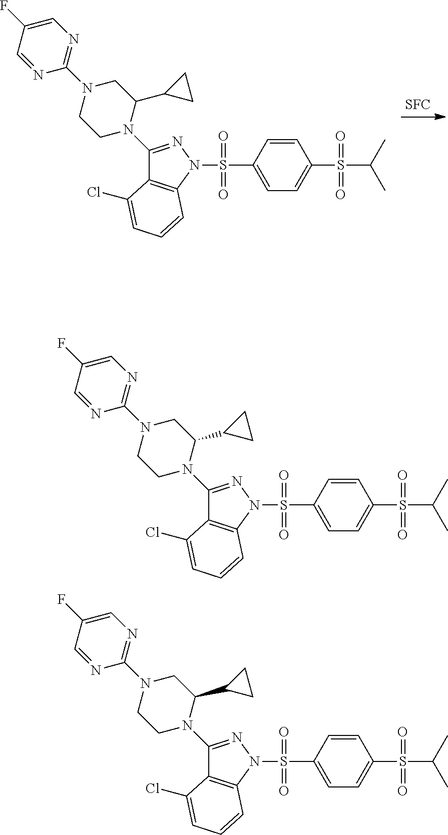 [SMILES: CC(C)S(=O)(=O)c1ccc(S(=O)(=O)n2nc(N3CCN(c4ncc(F)cn4)CC3C3CC3)c3c(Cl)cccc32)cc1 ; "SFC" ; CC(C)S(=O)(=O)c1ccc(S(=O)(=O)n2nc(N3CCN(c4ncc(F)cn4)C[C@@H]3C3CC3)c3c(Cl)cccc32)cc1 ; CC(C)S(=O)(=O)c1ccc(S(=O)(=O)n2nc(N3CCN(c4ncc(F)cn4)C[C@H]3C3CC3)c3c(Cl)cccc32)cc1]

Step 1: 4-Chloro-3-[2-cyclopropyl-4-(5-fluoropyrimidin-2-yl)piperazin-1-yl]-1-(4-isopropylsulfonylphenyl)sulfonyl-indazole (160 mg, 258 μmol, 1 eq) was separated by chrial SFC (column: REGIS(S,S)WHELK-O1(250 mm*25 mm, 10 μm); mobile phase: [0.1% NH₃·H₂O ETOH]; B %: 44%-44%, 10 min) to give two isomers. The structures were assigned randomly. 4-Chloro-3-[(2S)-2-cyclopropyl-4-(5-fluoropyrimidin-2-yl)piperazin-1-yl]-1-(4-isopropylsulfonylphenyl)sulfonyl-indazole (68.6 mg, 109 μmol, 42.2% yield, 98.43% purity, Rt=1.910 minutes; E.E. by chiral HPLC (%)=99.50%) was isolated as a white solid. ¹H NMR (DMSO-d₆) δ 8.49 (s, 2H), 8.07-8.18 (m, 3H), 8.04 (d, 2H), 7.67 (m, 1H), 7.49 (d, 1H), 4.35-4.51 (m, 2H), 3.57-3.66 (m, 1H), 3.40-3.54 (m, 2H), 3.16-3.28 (m, 2H), 2.99-3.11 (m, 1H), 1.08 (m, 7H), 0.07-0.18 (m, 1H), −0.03 (m, 1H), −0.52-−0.41 (m, 1H), −0.98 (m, 1H). HPLC: 98.43% (220 nm), 97.27% (215 nm), 99.16% (254 nm). MS (ESI): mass calcd. For C₂₇H₂₈N₆S₂O₄ClF 618.13 mass found 619.0 [M+H]⁺. 4-Chloro-3-[(2R)-2-cyclopropyl-4-(5-fluoropyrimidin-2-yl)piperazin-1-yl]-1-(4-isopropylsulfonylphenyl)sulfonyl-indazole (70.1 mg, 110 μmol, 42.74% yield, 97.54% purity, Rt=1.666 minutes; E.E. by chiral HPLC (%)=100.0%) was isolated as a white solid. ¹H NMR (DMSO-d₆) δ 8.49 (s, 2H), 8.10-8.17 (m, 3H), 8.04 (d, 2H), 7.67 (m, 1H), 7.49 (d, 1H), 4.43 (br d, 2H), 3.56-3.67 (m, 1H), 3.41-3.55 (m, 2H), 3.19-3.29 (m, 2H), 3.01-3.09 (m, 1H), 0.99-1.12 (m, 7H), 0.08-0.20 (m, 1H), −0.08-0.03 (m, 1H), −0.51-−0.40 (m, 1H), −0.98 (m, 1H). HPLC: 97.54% (220 nm), 96.92% (215 nm), 98.23% (254 nm). MS (ESI): mass calcd. For C₂₇H₂₈N₆S₂O₄ClF 618.13 mass found 619.0 [M+H]⁺.

### Compound 62: 4-chloro-1-(3-fluoro-4-isopropylsulfonyl-phenyl) sulfonyl-3-[7-(5-fluoropyrimidin-2-yl)-4, 7-diazaspiro [2.5]octan-4-yl]indazole

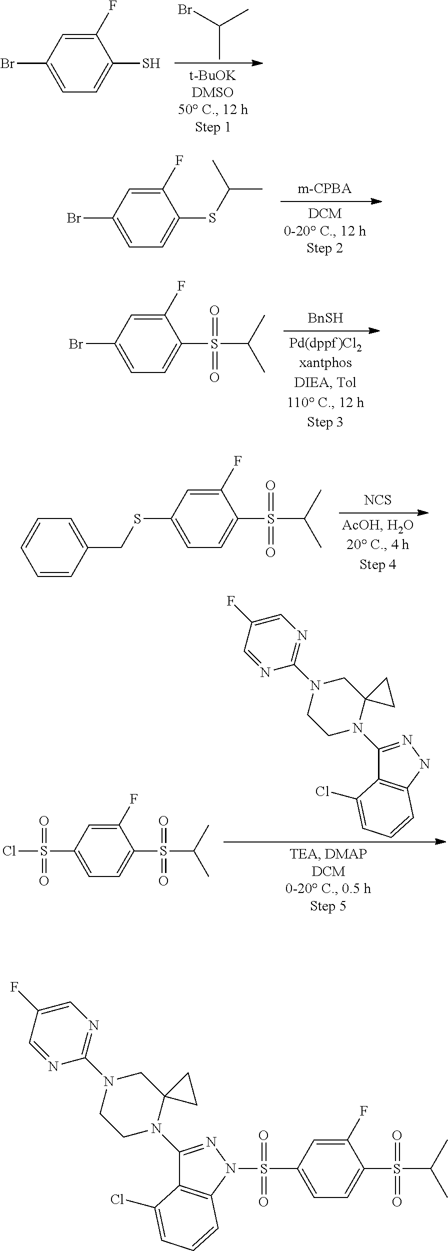

Step 1: To a solution of 4-bromo-2-fluoro-benzenethiol (3 g, 14.5 mmol, 1 eq) in THF (30 mL) was added K₂CO₃ (6.01 g, 43.5 mmol, 3 eq) and 2-bromopropane (2.67 g, 21.7 mmol, 2.04 mL, 1.5 eq). The mixture was stirred at 50° C. for 12 hours. TLC indicated 4-bromo-2-fluoro-benzenethiol was consumed completely and one new spot formed. The reaction was clean according to TLC. The reaction mixture was added to water (20 mL), extracted with EtOAc (10 mL*3). The combined organic layers were washed with brine 20 mL and dried over Na₂SO₄, filtered and concentrated under reduced pressure to give desired 4-bromo-2-fluoro-1-isopropylsulfanyl-benzene (3.6 g, crude) as a white solid.

Step 2: To a solution of 4-bromo-2-fluoro-1-isopropylsulfanyl-benzene (3.6 g, 14.5 mmol, 1 eq) in DCM (40 mL) was added m-CPBA (8.80 g, 43.4 mmol, 85% purity, 3 eq) at 0° C. The mixture was stirred at 20° C. for 12 hours. TLC (petroleum ether/ethyl acetate=3/1) indicated 4-bromo-2-fluoro-1-isopropylsulfanyl-benzene was consumed completely and one new spot formed. The reaction was clean according to TLC. Then it was partitioned between of sat.aq.Na₂SO₃ (30 mL) and DCM (100 mL). The organic phase was separated, washed with sat. Na₂SO₃ (30 mL), brine (30 mL), dried over Na₂SO₄, filtered and concentrated under reduced pressure to give desired 4-bromo-2-fluoro-1-isopropylsulfonyl-benzene (4 g, crude) as a colourless oil.

Step 3: A mixture of phenylmethanethiol (1.46 g, 11.7 mmol, 1.38 mL, 1.1 eq), 4-bromo-2-fluoro-1-isopropylsulfonyl-benzene (3 g, 10.7 mmol, 1 eq), DIEA (2.76 g, 21.3 mmol, 3.72 mL, 2 eq), Xantphos (617 mg, 1.07 mmol, 0.1 eq) and Pd(dppf)Cl₂ (195 mg, 267 μmol, 0.025 eq) in Tol. (30 mL) was degassed and purged with N₂ for 3 times, and then the mixture was stirred at 110° C. for 12 hours under N₂ atmosphere. TLC (petroleum ether/ethyl acetate=3/1) indicated 4-bromo-2-fluoro-1-isopropylsulfonyl-benzene was consumed completely and one new spot formed. The reaction was clean according to TLC. The reaction mixture was concentrated under reduced pressure to remove Tol. The reaction mixture was added to water (50 mL), extracted with EtOAc (30 mL*3). The combined organic layers were washed with brine (20 mL) and dried over Na₂SO₄, filtered and concentrated under reduced pressure to give a residue. The residue was purified by column chromatography (ISCO®; 40 g SepaFlash® Silica Flash Column, Eluent of 0˜20% Ethyl acetate/petroleum ether gradient @50 mL/min) to give desired 4-benzylsulfanyl-2-fluoro-1-isopropylsulfonyl-benzene (3.4 g, crude) as an orange oil.

Step 4: To a solution of 4-benzylsulfanyl-2-fluoro-1-isopropylsulfonyl-benzene (3.4 g, 10.5 mmol, 1 eq) in AcOH (20 mL) and H₂O (4 mL) was added NCS (4.20 g, 31.4 mmol, 3 eq). The mixture was stirred at 20° C. for 4 hours. TLC (petroleum ether/ethyl acetate=3/1) showed 4-benzylsulfanyl-2-fluoro-1-isopropylsulfonyl-benzene was consumed completely and one major new spot with larger polarity was detected. The reaction mixture was diluted with water 50 mL and extracted with EtOAc (50 mL*3). The combined organic layers were washed with brine 50 mL, dried over anhydrous Na₂SO₄, filtered and concentrated under reduced pressure to give a residue. The residue was purified by column chromatography (ISCO®; 40 g SepaFlash® Silica Flash Column, Eluent of 0˜20% Ethyl acetate/petroleum ether gradient @50 mL/min) to give desired 3-fluoro-4-isopropylsulfonyl-benzenesulfonyl chloride (580 mg, crude) as a white solid.

Step 5: To a solution of 4-chloro-3-[7-(5-fluoropyrimidin-2-yl)-4, 7-diazaspiro [2.5]octan-4-yl]-1H-indazole (25 mg, 69.9 μmol, 1 eq) and 3-fluoro-4-isopropylsulfonyl-benzenesulfonyl chloride (62.9 mg, 209 μmol, 3 eq) in DCM (2 mL) was added TEA (14.1 mg, 139 μmol, 19.4 uL, 2 eq) and DMAP (851 pg, 6.97 μmol, 0.1 eq) at 0° C. The mixture was stirred at 20° C. for 0.5 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (Silica gel, petroleum ether/ethyl acetate=2/1) to give desired 4-chloro-1-(3-fluoro-4-isopropylsulfonyl-phenyl) sulfonyl-3-[7-(5-fluoropyrimidin-2-yl)-4, 7-diazaspiro [2.5]octan-4-yl]indazole (17.7 mg, 27.8 μmol, 39.9% yield, 97.83% purity) as a white solid. H NMR (400 MHz, DMSO-d₆) δ 8.53 (s, 2H), 8.13 (d, J=8.4 Hz, 1H), 8.08-8.01 (m, 2H), 7.96-7.88 (m, 1H), 7.76-7.67 (m, 1H), 7.55 (d, J=7.8 Hz, 1H), 4.00 (s, 2H), 3.63 (br s, 4H), 3.55-3.49 (m, 1H), 1.19 (d, J=6.9 Hz, 6H), 0.85 (br s, 2H), 0.47 (br s, 2H). HPLC: 97.83% (220 nm), 98.63% (210 nm), 99.69% (254 nm). MS (ESI): mass calcd. For C₂₆H₂₅ClF₂N₆O₄S₂ 622.10, mass found 622.9 [M+H]⁺.

### Compound 63: 4-chloro-3-(2-cyclopropyl-1-piperidyl)-1-(4-isopropylsulfonylphenyl) sulfonyl-indazole

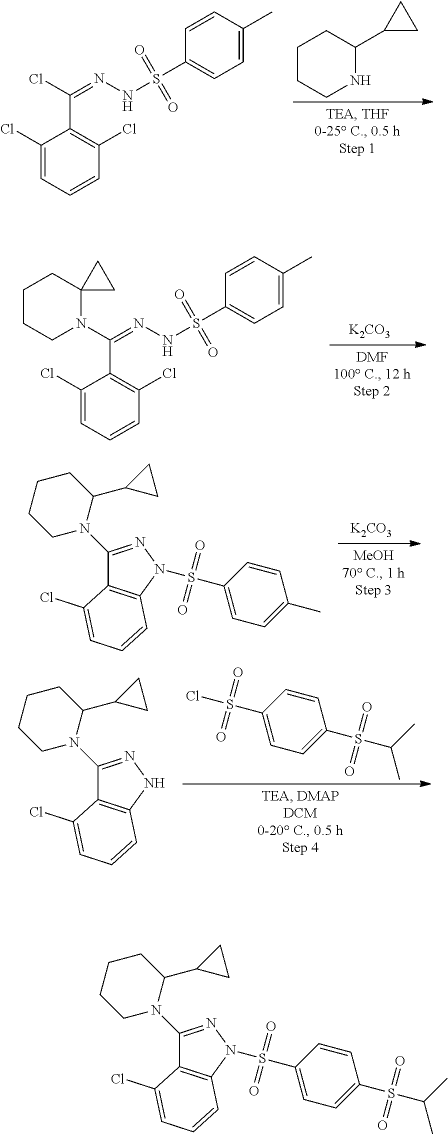

Step 1: To a solution of 2-cyclopropylpiperidine (50 mg, 309 μmol, 1 eq, HCl) in THF (3 mL) was added dropwise TEA (313 mg, 3.09 mmol, 430 μL, 10 eq) at 25° C. After addition, the mixture was stirred at this temperature for 10 mins, and then (1Z)-2,6-dichloro-N-(p-tolylsulfonyl)benzohydrazonoyl chloride (128 mg, 340 μmol, 1.1 eq) in THF (3 mL) was added dropwise at 0° C. The resulting mixture was stirred at 25° C. for 20 mins. LC-MS showed 2-cyclopropylpiperidine was consumed completely and one main peak with desired mass was detected. Then it was separated between (20 mL) of water and (40 mL) of ethyl acetate. The organic phase was separated, washed with brine (30 mL), dried over Na₂SO₄, filtered and concentrated under reduced pressure to give desired N-[(E)-[(2-cyclopropyl-1-piperidyl)-(2, 6-dichlorophenyl) methylene]amino]-4-methyl-benzenesulfonamide (145 mg, crude) as a yellow solid. MS (ESI): mass calcd. For C₂₂H₂₅Cl₂N₃O₂S 465.10, mass found 466.1 [M+H]⁺.

Step 2: To a solution of N-[(E)-[(2-cyclopropyl-1-piperidyl)-(2, 6-dichlorophenyl) methylene]amino]-4-methyl-benzenesulfonamide (145 mg, 311 μmol, 1 eq) in DMF (5 mL) was added K₂CO₃ (172 mg, 1.24 mmol, 4 eq). The mixture was stirred at 100° C. for 12 hours. LC-MS showed N-[(E)-[(2-cyclopropyl-1-piperidyl)-(2, 6-dichlorophenyl) methylene]amino]-4-methyl-benzenesulfonamide was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give desired 4-chloro-3-(2-cyclopropyl-1-piperidyl)-1-(p-tolylsulfonyl) indazole (130 mg, crude) as an orange oil. MS (ESI): mass calcd. For C₂₂H₂₄ClN₃O₂S 429.13, mass found 430.0 [M+H]⁺.

Step 3: To a solution of 4-chloro-3-(2-cyclopropyl-1-piperidyl)-1-(p-tolylsulfonyl) indazole (130 mg, 302 μmol, 1 eq) in MeOH (5 mL) was added K₂CO₃ (209 mg, 1.51 mmol, 5 eq). The mixture was stirred at 70° C. for 1 hour. LC-MS showed 4-chloro-3-(2-cyclopropyl-1-piperidyl)-1-(p-tolylsulfonyl) indazole was consumed completely and desired compound was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by by prep-TLC (Silica gel, petroleum ether/=3/1) to give desired 4-chloro-3-(2-cyclopropyl-1-piperidyl)-1H-indazole (80 mg, crude) as a yellow oil. MS (ESI): mass calcd. For C₁₅H₁₈ClN₃ 275.12, mass found 276.0 [M+H]⁺.

Step 4: To a solution of 4-isopropylsulfonylbenzenesulfonyl chloride (123 mg, 435 μmol, 1.5 eq) and 4-chloro-3-(2-cyclopropyl-1-piperidyl)-1H-indazole (80 mg, 290 μmol, 1 eq) in DCM (2 mL) was added TEA (58.7 mg, 580 μmol, 80.8 μL, 2 eq) and DMAP (3.54 mg, 29.0 μmol, 0.1 eq) at 0° C. The mixture was stirred at 20° C. for 0.5 hour. LC-MS showed 4-chloro-3-(2-cyclopropyl-1-piperidyl)-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water (NH₄HCO₃)-ACN]; B %: 55%-85%, 8 min) to give desired 4-chloro-3-(2-cyclopropyl-1-piperidyl)-1-(4-isopropylsulfonylphenyl) sulfonyl-indazole (21.4 mg, 40.9 μmol, 14.1% yield, 99.74% purity) as a white solid. ¹H NMR (400 MHz, DMSO-d₆) δ 8.14-8.08 (m, 3H), 8.07-8.02 (m, 2H), 7.64 (t, J=8.1 Hz, 1H), 7.45 (d, J=7.8 Hz, 1H), 3.52-3.45 (m, 1H), 3.44-3.34 (m, 3H), 3.32-3.31 (m, 1H), 3.14 (br d, J=12.5 Hz, 1H), 2.88-2.80 (m, 1H), 1.90-1.80 (m, 1H), 1.77-1.65 (m, 3H), 1.63-1.56 (m, 1H), 1.54-1.44 (m, 1H), 1.27-1.17 (m, 1H), 1.08 (br d, J=1.8 Hz, 3H), 0.21-0.09 (m, 1H), −0.21 (br dd, J=4.8, 9.4 Hz, 1H), −0.41-−0.55 (m, 1H), −1.07 (br dd, J=4.5, 9.3 Hz, 1H). HPLC: 99.74% (220 nm), 99.88% (210 nm), 100.00% (254 nm). MS (ESI): mass calcd. For C₂₄H₂₈ClN₃O₄S₂ 521.12, mass found 522.1 [M+H]⁺.

### Compound 64: 4-chloro-3-[7-(5-fluoropyrimidin-2-yl)-4, 7-diazaspiro [2.5]octan-4-yl]-1-(2, 3, 5, 6-tetradeuterio-4-isopropylsulfonyl-phenyl) sulfonyl-indazole

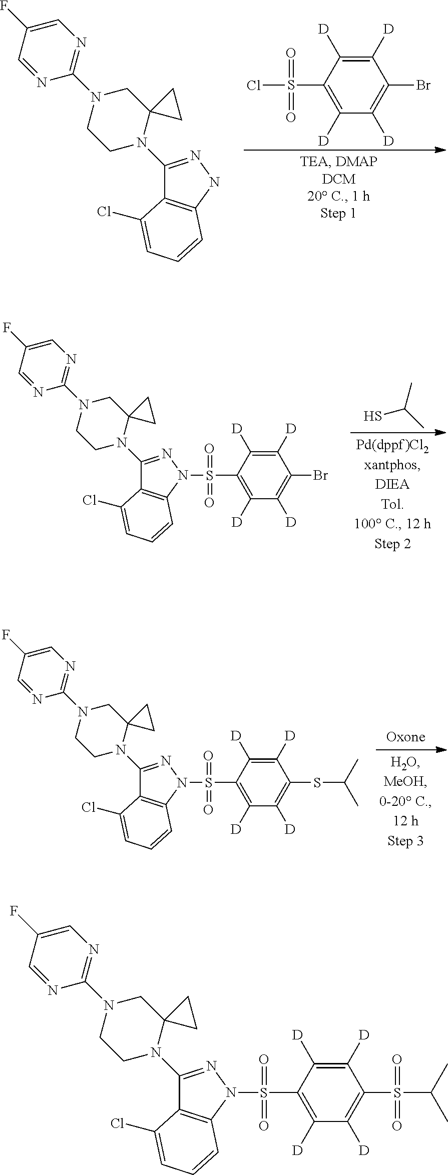

Step 1: To a solution of 4-chloro-3-[7-(5-fluoropyrimidin-2-yl)-4,7-diazaspiro[2.5]octan-4-yl]-1H-indazole (50.0 mg, 139 μmol, 1 eq) and 4-bromo-2,3,5,6-tetradeuterio-benzenesulfonyl chloride (43.4 mg, 167 μmol, 1.2 eq) in DCM (2.00 mL) was added TEA (28.2 mg, 278 μmol, 38.7 μL, 2 eq) and DMAP (1.70 mg, 13.9 μmol, 0.1 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole was consumed completely and one main peak with desired mass was detected. The reaction mixture was add to water (20 mL) and extracted with DCM (10 mL*3). The combined organic layers were washed with brine (20 mL), dried over Na₂SO₄, filtered and concentrated under reduced pressure to give a residue. The residue was purified by prep-TLC (Silica gel, petroleum ether/ethyl acetate=3/1) to give desired 1-(4-bromo-2, 3, 5, 6-tetradeuterio-phenyl) sulfonyl-4-chloro-3-[7-(5-fluoropyrimidin-2-yl)-4, 7-diazaspiro [2.5]octan-4-yl]indazole (110 mg, crude) as a light yellow oil. MS (ESI): mass calcd. For C₂₃H₁₅N₆BrSO₂ClFD₄ 580.04 mass found 585.1 [M+H]⁺.

Step 2: A mixture of 1-(4-bromo-2,3,5,6-tetradeuterio-phenyl)sulfonyl-4-chloro-3-[7-(5-fluoropyrimidin-2-yl)-4,7-diazaspiro[2.5]octan-4-yl]indazole (30.0 mg, 51.5 μmol, 1 eq), propane-2-thiol (19.6 mg, 257 μmol, 24.1 μL, 5 eq), Pd(dppf)Cl₂ (3.77 mg, 5.16 μmol, 0.1 eq), Xantphos (5.97 mg, 10.3 μmol, 0.2 eq) and DIEA (13.3 mg, 103 μmol, 17.9 μL, 2 eq) in Tol. (2.00 mL) was degassed and purged with N₂ for 3 times, and then the mixture was stirred at 100° C. for 12 hours under N₂ atmosphere. LC-MS showed 1-(4-bromo-2, 3, 5, 6-tetradeuterio-phenyl) sulfonyl-4-chloro-3-[7-(5-fluoropyrimidin-2-yl)-4, 7-diazaspiro [2.5]octan-4-yl]indazole was consumed completely and one main peak with desired mass was detected. The reaction mixture was partitioned between H₂O (20.0 mL) and EtOAc (10.0 mL). The organic phase was separated, washed with brine (20.0 mL) dried over Na₂SO₄, filtered and concentrated under reduced pressure to give a residue. The residue was purified by prep-TLC (Silica gel, petroleum ether/ethyl acetate=3/1) to give desired 4-chloro-3-[7-(5-fluoropyrimidin-2-yl)-4,7-diazaspiro[2.5]octan-4-yl]-1-(2,3,5,6-tetradeuterio-4-isopropylsulfanyl-phenyl)sulfonyl-indazole (37 mg, crude) as a yellow oil. MS (ESI): mass calcd. For C₂₆H₂₂N₆S₂O₂ClFD₄ 576.15 mass found 577.2 [M+H]⁺.

Step 3: To a solution of 4-chloro-3-[7-(5-fluoropyrimidin-2-yl)-4,7-diazaspiro[2.5]octan-4-yl]-1-(2,3,5,6-tetradeuterio-4-isopropylsulfanyl-phenyl)sulfonyl-indazole (25.0 mg, 43.3 μmol, 1 eq) in MeOH (0.5 mL) was added Oxone (39.9 mg, 65.0 μmol, 1.5 eq) in H₂O (0.5 mL) at 0° C. The mixture was stirred at 20° C. for 12 hours. LC-MS showed 4-chloro-3-[7-(5-fluoropyrimidin-2-yl)-4,7-diazaspiro[2.5]octan-4-yl]-1-(2,3,5,6-tetradeuterio-4-isopropylsulfanyl-phenyl)sulfonyl-indazole was consumed completely and desired mass was detected. The reaction mixture was added to H₂O (20.0 mL), extracted with EtOAc (10 mL*3). The combined organic layers were washed with brine (20.0 mL) and dried over Na₂SO₄, filtered and concentrated under reduced pressure to give a residue. The residue was purified by prep-HPLC (TFA condition; column: Phenomenex Luna C18 75*30 mm*3 μm; mobile phase: [water(TFA)-ACN]; B %: 55%-85%, 8 min) to give desired 4-chloro-3-[7-(5-fluoropyrimidin-2-yl)-4,7-diazaspiro[2.5]octan-4-yl]-1-(2,3,5,6-tetradeuterio-4-isopropylsulfonyl-phenyl)sulfonyl-indazole (4 mg, 6.57 μmol, 15.2% yield, 100.00% purity) as a yellow solid. ¹H NMR (400 MHz, DMSO-d₆) δ 8.47 (s, 2H), 8.09 (d, J=8.5 Hz, 1H), 7.67 (t, J=8.1 Hz, 1H), 7.49 (d, J=7.6 Hz, 1H), 3.93 (s, 2H), 3.67-3.58 (m, 2H), 3.55 (br s, 3H), 1.08 (d, J=6.8 Hz, 6H), 0.77 (br s, 2H), 0.35 (br s, 2H). HPLC: 95.47% (220 nm), 95.62% (215 nm), 100.00% (254 nm). MS (ESI): mass calcd. For C₂₆H₂₂N₆S₂O₄ClFD₄ 608.14 mass found 609.0 [M+H]⁺.

### Compound 65: 4-chloro-1-(4-isopropylsulfonylphenyl) sulfonyl-3-(7-pyrimidin-2-yl-4,7-diazaspiro[2.5]octan-4-yl) indazole

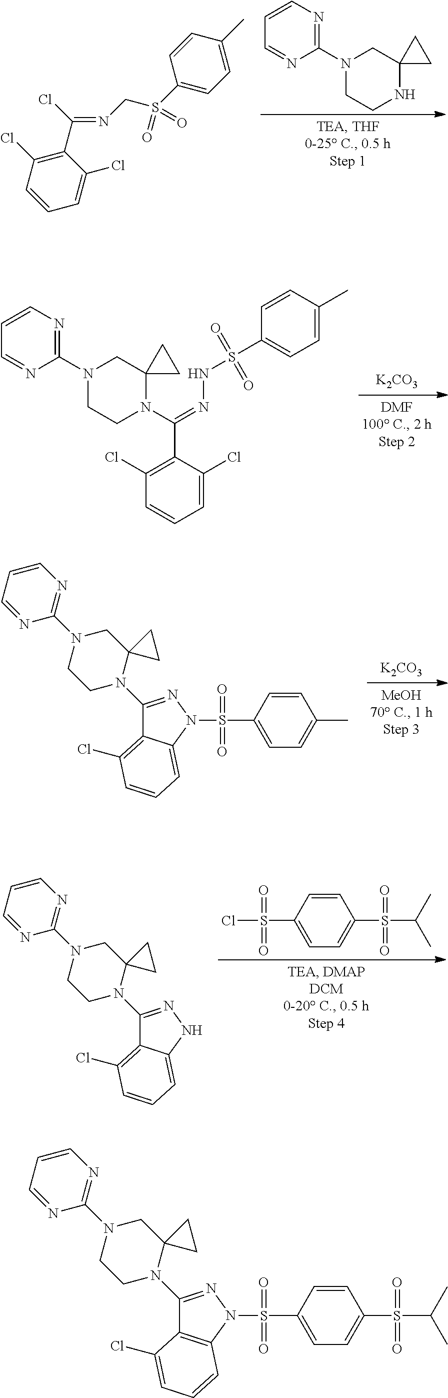

Step 1: To a solution of 7-pyrimidin-2-yl-4, 7-diazaspiro [2.5]octane (229 mg, 1.01 mmol, 1 eq, HCl) in THF (3 mL) was added dropwise TEA (1.02 g, 10.1 mmol, 1.41 mL, 10 eq) at 25° C. After addition, the mixture was stirred at this temperature for 10 mins, and then (1Z)-2,6-dichloro-N-(p-tolylsulfonyl)benzohydrazonoyl chloride (420 mg, 1.11 mmol, 1.1 eq) in THF (3 mL) was added dropwise at 0° C. The resulting mixture was stirred at 25° C. for 20 mins. LC-MS showed 7-pyrimidin-2-yl-4, 7-diazaspiro [2.5]octane was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(Z)-[(2,6-dichlorophenyl)-(7-pyrimidin-2-yl-4,7-diazaspiro[2.5]octan-4-yl)methylene]amino]-4-methyl-benzenesulfonamide (540 mg, crude) as a yellow solid. MS (ESI): mass calcd. For C₂₄H₂₄Cl₂N₆O₂S 530.11, mass found 531.2 [M+H]⁺.

Step 2: To a solution of N—[(Z)-[(2, 6-dichlorophenyl)-(7-pyrimidin-2-yl-4, 7-diazaspiro [2.5]octan-4-yl) methylene]amino]-4-methyl-benzenesulfonamide (540 mg, 1.02 mmol, 1 eq) in DMF (20 mL) was added K₂CO₃ (562 mg, 4.06 mmol, 4 eq). The mixture was stirred at 100° C. for 2 hours. LC-MS showed N—[(Z)-[(2, 6-dichlorophenyl)-(7-pyrimidin-2-yl-4, 7-diazaspiro [2.5]octan-4-yl) methylene]amino]-4-methyl-benzenesulfonamide was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give desired 4-chloro-1-(p-tolylsulfonyl)-3-(7-pyrimidin-2-yl-4,7-diazaspiro[2.5]octan-4-yl) indazole (500 mg, crude) as an orange oil. MS (ESI): mass calcd. For C₂₄H₂₃ClN₆O₂S 494.13, mass found 495.2 [M+H]⁺.

Step 3: To a solution of 4-chloro-1-(p-tolylsulfonyl)-3-(7-pyrimidin-2-yl-4, 7-diazaspiro [2.5]octan-4-yl) indazole (500 mg, 1.01 mmol, 1 eq) in MeOH (20 mL) was added K₂CO₃ (698 mg, 5.05 mmol, 5 eq). The mixture was stirred at 70° C. for 1 hour. LC-MS showed 4-chloro-1-(p-tolylsulfonyl)-3-(7-pyrimidin-2-yl-4, 7-diazaspiro [2.5]octan-4-yl) indazole was consumed completely and desired compound was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-TLC (Silica gel, petroleum ether/ethyl acetate=3/1) to give desired 4-chloro-3-(7-pyrimidin-2-yl-4, 7-diazaspiro [2.5]octan-4-yl)-1H-indazole (180 mg, crude) as a yellow solid. MS (ESI): mass calcd. For C₁₇H₁₇ClN₆ 340.12, mass found 341.0 [M+H]⁺.

Step 4: To a solution of 4-isopropylsulfonylbenzenesulfonyl chloride (62.2 mg, 220 μmol, 1.5 eq) and 4-chloro-3-(7-pyrimidin-2-yl-4,7-diazaspiro[2.5]octan-4-yl)-1H-indazole (50 mg, 147 μmol, 1 eq) in DCM (5 mL) was added TEA (29.7 mg, 293 μmol, 40.8 μL, 2 eq) and DMAP (1.79 mg, 14.7 μmol, 0.1 eq) at 0° C. The mixture was stirred at 20° C. for 0.5 hours. LC-MS showed 4-chloro-3-(7-pyrimidin-2-yl-4, 7-diazaspiro [2.5]octan-4-yl)-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water(NH₄HCO₃)-ACN]; B %: 50%-80%, 8 min) to give desired 4-chloro-1-(4-isopropylsulfonylphenyl) sulfonyl-3-(7-pyrimidin-2-yl-4,7-diazaspiro[2.5]octan-4-yl) indazole (15.5 mg, 26.4 μmol, 18.0% yield, 99.86% purity) as a pale yellow solid. ¹H NMR (400 MHz, DMSO-d₆) δ 8.43 (d, J=4.6 Hz, 2H), 8.17-8.06 (m, 5H), 7.73 (t, J=8.1 Hz, 1H), 7.55 (d, J=7.6 Hz, 1H), 6.71 (t, J=4.7 Hz, 1H), 4.03 (s, 2H), 3.72 (br d, J=1.2 Hz, 2H), 3.59 (br d, J=4.3 Hz, 2H), 3.55-3.48 (m, 1H), 1.13 (d, J=6.8 Hz, 6H), 0.82 (s, 2H), 0.40 (br s, 2H). HPLC: 99.86% (220 nm), 100.00% (210 nm), 100.00% (254 nm). MS (ESI): mass calcd. For C₂₆H₂₇ClN₆O₄S₂ 586.12, mass found 587.1 [M+H]⁺.

### Compound 66: 4-chloro-1-(4-isopropylsulfonylphenyl)sulfonyl-3-[2-(trifluoromethyl)-1-piperidyl]indazole

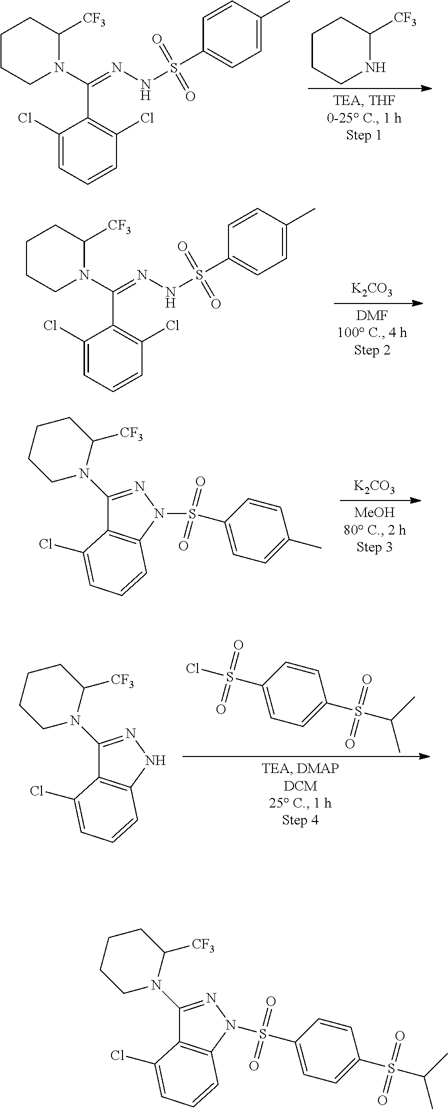

Step 1: To a solution of (1E)-2, 6-dichloro-N-(p-tolylsulfonyl)benzohydrazonoyl chloride (111 mg, 294 μmol, 1.5 eq) and 2-(trifluoromethyl)piperidine (30 mg, 196 μmol, 1 eq) in THF (5 mL) was added TEA (198 mg, 1.96 mmol, 273 μL, 10 eq) at 0° C. The mixture was stirred at 25° C. for 1 hour. LC-MS showed (1E)-2, 6-dichloro-N-(p-tolylsulfonyl) benzohydrazonoyl chloride was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N-[(E)-[(2,6-dichlorophenyl)-[2-(trifluoromethyl)-1-piperidyl]methylene]amino]-4-methyl-benzenesulfonamide (60 mg, crude) as a yellow oil. MS (ESI): mass calcd. For C₂₀H₂₁ClF₃N₃O₂S 459.1 mass found 460.1 [M+H]⁺.

Step 2: To a solution of N-[(E)-[(2, 6-dichlorophenyl)-[2-(trifluoromethyl)-1-piperidyl]methylene]amino]-4-methyl-benzenesulfonamide (590 mg, 1.19 mmol, 1 eq) in DMF (6 mL) was added K₂CO₃ (825 mg, 5.97 mmol, 5 eq). The mixture was stirred at 100° C. for 4 hours. LC-MS showed N-[(E)-[(2, 6-dichlorophenyl)-[2-(trifluoromethyl)-1-piperidyl]methylene]amino]-4-methyl-benzenesulfonamide was consumed completely and desired mass was detected. The reaction mixture was partitioned between 5 mL of H₂O and 5 mL of EtOAc. The organic phase was separated, washed with 50 mL of brine, dried over Na₂SO₄, filtered and concentrated under reduced pressure to give desired 4-chloro-1-(p-tolylsulfonyl)-3-[2-(trifluoromethyl)-1-piperidyl]indazole (550 mg, crude) as a yellow oil. MS (ESI): mass calcd. For C₂₀H₁₉ClF₃N₃O₂S 457.1 mass found 458.1 [M+H]⁺.

Step 3: To a solution of 4-chloro-1-(p-tolylsulfonyl)-3-[2-(trifluoromethyl)-1-piperidyl]indazole (550 mg, 1.20 mmol, 1 eq) in MeOH (6 mL) was added K₂CO₃ (830 mg, 6.01 mmol, 5 eq). The mixture was stirred at 80° C. for 2 hours. LC-MS showed 4-chloro-1-(p-tolylsulfonyl)-3-[2-(trifluoromethyl)-1-piperidyl]indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (Silica gel, petroleum ether/ethyl acetate=3/1) to give desired 4-chloro-3-[2-(trifluoromethyl)-1-piperidyl]-1H-indazole (160 mg, 527 μmol, 43.86% yield) as a white solid. MS (ESI): mass calcd. For C₁₃H₁₃ClF₃N₃ 303.1 mass found 304.1 [M+H]⁺.

Step 4: To a solution of 4-chloro-3-[2-(trifluoromethyl)-1-piperidyl]-1H-indazole (50 mg, 165 μmol, 1 eq) and 4-isopropylsulfonylbenzenesulfonyl chloride (69.8 mg, 247 μmol, 1.5 eq) in DCM (1 mL) was added TEA (33.3 mg, 329 μmol, 45.8 μL, 2 eq) and DMAP (2.01 mg, 16.5 μmol, 0.1 eq). The mixture was stirred at 25° C. for 1 hour. LC-MS showed 4-chloro-3-[2-(trifluoromethyl)-1-piperidyl]-1H-indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-HPLC (FA condition; Method: column: Phenomenex Luna C18 75*30 mm*3 μm; mobile phase: [water (TFA)-ACN]; B %: 50%-80%, 8 min) to give desired 4-chloro-1-(4-isopropylsulfonylphenyl)sulfonyl-3-[2-(trifluoromethyl)-1-piperidyl]indazole (9 mg, 16.3 μmol, 9.92% yield, 99.84% purity) as a white solid. ¹H NMR (400 MHz, DMSO-d₆) δ=9.72 (s, 1H), 8.02 (d, J=8.3 Hz, 2H), 7.86 (d, J=8.3 Hz, 2H), 7.44 (br d, J=7.5 Hz, 1H), 7.38-7.31 (m, 1H), 7.30-7.22 (m, 1H), 7.22-7.18 (m, 1H), 7.15-7.07 (m, 2H), 6.63 (d, J=7.5 Hz, 1H), 3.56-3.52 (m, 1H), 1.86 (s, 3H), 1.18 (d, J=6.8 Hz, 6H). HPLC: 99.84% (220 nm), 99.85% (215 nm), 99.70% (254 nm). MS (ESI): mass calcd. For C₂₃H₂₅ClF₃N₃O₃S₂ 547.1 mass found 548.1 [M+H]⁺.

### Compound 67: 4-chloro-3-[4-(5-fluoropyrimidin-2-yl)-2-(trifluoromethyl) piperazin-1-yl]-1-(4-isopropylsulfonylphenyl) sulfonyl-indazole

### Compound 68: 4-chloro-3-[(2R)-4-(5-fluoropyrimidin-2-yl)-2-(trifluoromethyl) piperazin-1-yl]-1-(4-isopropylsulfonylphenyl) sulfonyl-indazole

### Compound 69: 4-chloro-3-[(2S)-4-(5-fluoropyrimidin-2-yl)-2-(trifluoromethyl) piperazin-1-yl]-1-(4-isopropylsulfonylphenyl) sulfonyl-indazole

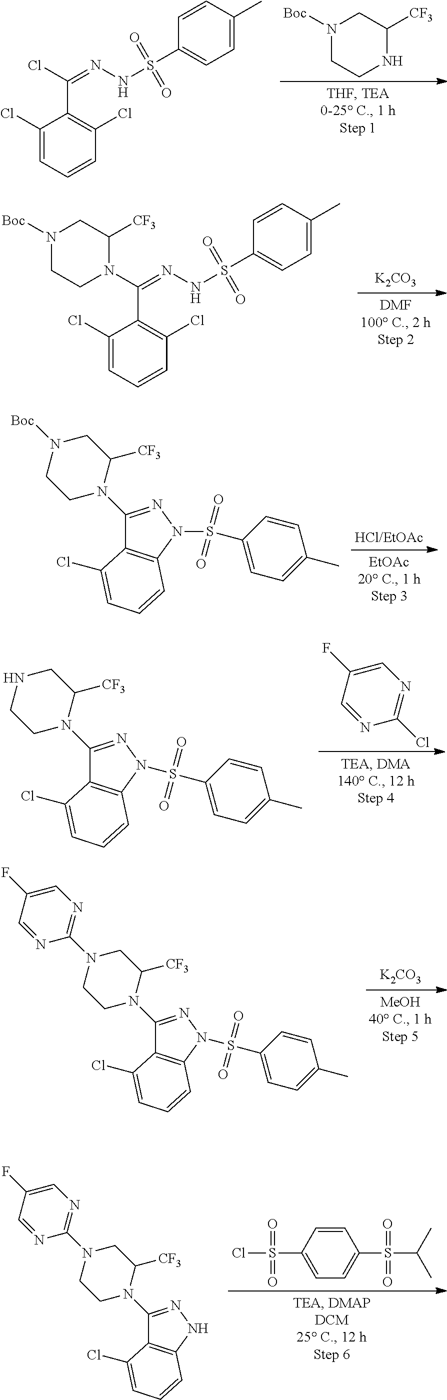

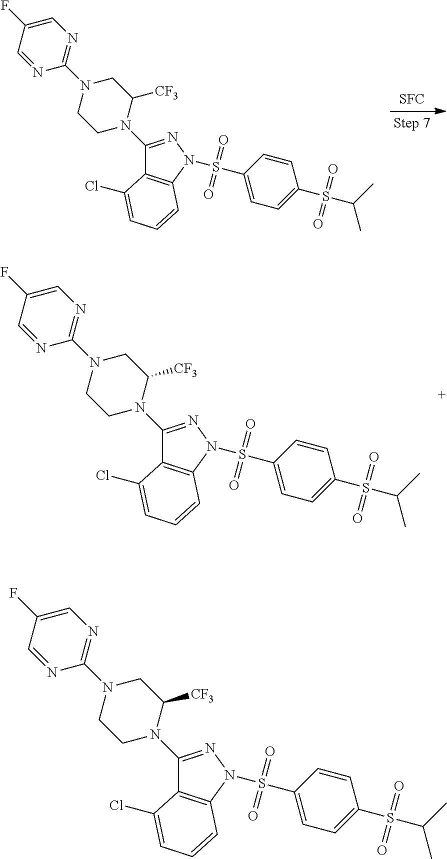 [SMILES: CC(C)S(=O)(=O)c1ccc(S(=O)(=O)n2nc(N3CCN(c4ncc(F)cn4)CC3C(F)(F)F)c3c(Cl)cccc32)cc1 ; "SFC" ; CC(C)S(=O)(=O)c1ccc(S(=O)(=O)n2nc(N3CCN(c4ncc(F)cn4)C[C@@H]3C(F)(F)F)c3c(Cl)cccc32)cc1 ; CC(C)S(=O)(=O)c1ccc(S(=O)(=O)n2nc(N3CCN(c4ncc(F)cn4)C[C@H]3C(F)(F)F)c3c(Cl)cccc32)cc1]

Step 1: To a solution of (1E)-2, 6-dichloro-N-(p-tolylsulfonyl) benzohydrazonoyl chloride (1.12 g, 2.96 mmol, 1.5 eq) and tert-butyl 3-(trifluoromethyl)piperazine-1-carboxylate (501 mg, 1.97 mmol, 1 eq) in THF (5 mL) was added TEA (1.99 g, 19.7 mmol, 2.74 mL, 10 eq) at 0° C. The mixture was stirred at 25° C. for 1 hour. LCMS showed (1E)-2, 6-dichloro-N-(p-tolylsulfonyl) benzohydrazonoyl chloride was consumed completely and desired mass was detected. The reaction mixture was partitioned between H₂O (10 mL) and EtOAc (30 mL). The organic phase was separated, washed with brine (50 mL), dried over Na₂SO₄, filtered and concentrated under reduced pressure to give desired tert-butyl 4-[(E)-C-(2,6-dichlorophenyl)-N-(p-tolylsulfonylamino)carbonimidoyl]-3-(trifluoromethyl)piperazine-1-carboxylate (1.17 g, crude). MS (ESI): mass calcd. For C₂₄H₂₇N₄Cl₂SO₄F₃ 594.11 mass found 595.2 [M+H]⁺.

Step 2: The mixture of tert-butyl 4-[(E)-C-(2, 6-dichlorophenyl)-N-(p-tolylsulfonylamino) carbonimidoyl]-3-(trifluoromethyl) piperazine-1-carboxylate (1.6 g, 2.69 mmol, 1 eq) and K₂CO₃ (3.71 g, 26.9 mmol, 10 eq) in DMF (16 mL) was stirred at 100° C. for 2 hours. LCMS showed tert-butyl 4-[(E)-C-(2, 6-dichlorophenyl)-N-(p-tolylsulfonylamino) carbonimidoyl]-3-(trifluoromethyl) piperazine-1-carboxylate was consumed completely and desired mass was detected. The reaction was added water (50 mL) and extracted with MTBE (2*50 mL). The combined organics were concentrated to get a residue. The residue was purified by flash silica gel chromatography (ISCO®; 40 g SepaFlash® Silica Flash Column, Eluent of 0˜50% ethyl acetate/petroleum ether gradient @80 mL/min) to give desired tert-butyl 4-[4-chloro-1-(p-tolylsulfonyl) indazol-3-yl]-3-(trifluoromethyl) piperazine-1-carboxylate (800 mg, 1.43 mmol, 53.2% yield) was a yellow solid. MS (ESI): mass calcd. For C₂₄H₂₆N₄ClSO₄F₃ 558.13 mass found 503.1 [M+H−56]⁺.

Step 3: To the solution of tert-butyl 4-[4-chloro-1-(p-tolylsulfonyl) indazol-3-yl]-3-(trifluoromethyl)piperazine-1-carboxylate (800 mg, 1.43 mmol, 1 eq) in EtOAc (10 mL) was added HCl/EtOAc (4M, 3.58 mL, 10 eq) and the solution was stirred at 25° C. for 1 hour. TLC showed tert-butyl 4-[4-chloro-1-(p-tolylsulfonyl) indazol-3-yl]-3-(trifluoromethyl) piperazine-1-carboxylate was consumed completely. The reaction was concentrated to give desired 4-chloro-1-(p-tolylsulfonyl)-3-[2-(trifluoromethyl) piperazin-1-yl]indazole (710 mg, crude, HCl) as a yellow solid.

Step 4: The solution of 4-chloro-1-(p-tolylsulfonyl)-3-[2-(trifluoromethyl) piperazin-1-yl]indazole (710 mg, 1.43 mmol, 1 eq, HCl), 2-chloro-5-fluoro-pyrimidine (570 mg, 4.30 mmol, 3 eq) and DIPEA (1.85 g, 14.3 mmol, 10 eq) in DMA (10 mL) was stirred at 140° C. for 12 hours. LCMS showed 4-chloro-1-(p-tolylsulfonyl)-3-[2-(trifluoromethyl) piperazin-1-yl]indazole remained and desired mass was detected. The reaction was concentrated to give desired 4-chloro-3-[4-(5-fluoropyrimidin-2-yl)-2-(trifluoromethyl) piperazin-1-yl]-1-(p-tolylsulfonyl) indazole (795 mg, crude) as a black oil. MS (ESI): mass calcd. For C₂₃H₁₉N₆ClSO₂F₄ 554.09 mass found 555.1 [M+H]⁺.

Step 5: The solution of 4-chloro-3-[4-(5-fluoropyrimidin-2-yl)-2-(trifluoromethyl) piperazin-1-yl]-1-(p-tolylsulfonyl) indazole (700 mg, 1.26 mmol, 1 eq) and K₂CO₃ (872 mg, 6.31 mmol, 1.35 mL, 5 eq) in MeOH (10 mL) was stirred at 40° C. for 1 hour. LCMS showed 4-chloro-3-[4-(5-fluoropyrimidin-2-yl)-2-(trifluoromethyl) piperazin-1-yl]-1-(p-tolylsulfonyl) indazole was consumed completely and desired mass was detected. The residue was added water (30 mL) and extracted with MTBE (2*20 mL). The combined organics were concentrated to get a residue. The residue was purified by prep-TLC (Silica gel, petroleum ether/EtOAc=2/1) to give desired 4-chloro-3-[4-(5-fluoropyrimidin-2-yl)-2-(trifluoromethyl) piperazin-1-yl]-1H-indazole (260 mg, 649 μmol, 51.4% yield) as a white solid. MS (ESI): mass calcd. For C₁₆H₁₃N₆ClF₄ 400.08 mass found 401.1 [M+H]⁺.

Step 6: The solution of 4-chloro-3-[4-(5-fluoropyrimidin-2-yl)-2-(trifluoromethyl) piperazin-1-yl]-1H-indazole (260 mg, 649 μmol, 1 eq), TEA (328 mg, 3.24 mmol, 5 eq) and DMAP (7.93 mg, 64.9 μmol, 0.1 eq) in DCM (5 mL) was added 4-isopropylsulfonylbenzenesulfonyl chloride (275 mg, 973 μmol, 1.5 eq) and the solution was stirred at 25° C. for 12 hours. LCMS showed 4-chloro-3-[4-(5-fluoropyrimidin-2-yl)-2-(trifluoromethyl) piperazin-1-yl]-1H-indazole was consumed completely and desired mass was detected. The reaction was concentrated to get a residue. The residue was purified by prep-HPLC (column: Phenomenex Luna C18 150*30 mm*5 μm; mobile phase: [water(TFA)-ACN]; B %: 55%-75%, 8 min) to give desired 4-chloro-3-[4-(5-fluoropyrimidin-2-yl)-2-(trifluoromethyl)piperazin-1-yl]-1-(4-isopropylsulfonylphenyl)sulfonyl-indazole (93 mg, 131 μmol, 20.2% yield, 91.41% purity) as a white solid. ¹HNMR (DMSO-d₆) δ 8.52 (s, 2H), 8.06-8.17 (m, 3H), 7.97-8.03 (m, 2H), 7.71 (m, 1H), 7.55 (d, 1H), 4.86 (br d, 1H), 4.67-4.79 (m, 1H), 4.38-4.46 (m, 1H), 3.74-3.85 (m, 1H), 3.63-3.71 (m, 1H), 3.53 (br s, 1H), 3.44 (br d, 1H), 3.09-3.20 (m, 1H), 1.05 (s, 6H). HPLC: 91.41% (220 nm), 90.90% (215 nm), 96.10% (254 nm). MS (ESI): mass calcd. For C₂₅H₂₃N₆S₂O₄ClF₄ 646.08 mass found 647.0 [M+H]⁺.

Step 7: 4-chloro-3-[4-(5-fluoropyrimidin-2-yl)-2-(trifluoromethyl)piperazin-1-yl]-1-(4-isopropylsulfonylphenyl)sulfonyl-indazole (80 mg, 124 μmol, 1 eq) was separated by chrial SFC (column: (s,s) WHELK-O1 (250 mm*30 mm, 5 μm); mobile phase: [0.1% NH₃·H₂O MeOH]; B %: 45%-45%, 8 min) to give two isomers. The structures were assigned randomly. 4-Chloro-3-[(2R)-4-(5-fluoropyrimidin-2-yl)-2-(trifluoromethyl)piperazin-1-yl]-1-(4-isopropylsulfonylphenyl)sulfonyl-indazole (24.0 mg, 35.5 μmol, 28.71% yield, 95.71% purity; Rt=1.560 minutes; E.E. by chiral HPLC (%)=100.0%) was isolated as a white solid. ¹H NMR (DMSO-d₆) δ 8.52 (s, 2H), 8.06-8.17 (m, 3H), 7.97-8.03 (m, 2H), 7.71 (m, 1H), 7.55 (d, 1H), 4.86 (br d, 1H), 4.67-4.79 (m, 1H), 4.38-4.46 (m, 1H), 3.74-3.85 (m, 1H), 3.63-3.71 (m, 1H), 3.53 (br s, 1H), 3.44 (br d, 1H), 3.09-3.20 (m, 1H), 1.05 (s, 6H). HPLC: 95.71% (220 nm), 95.45% (215 nm), 100.0% (254 nm). MS (ESI): mass calcd. For C₂₅H₂₃N₆S₂O₄ClF₄ 646.08 mass found 647.0 [M+H]⁺. 4-Chloro-3-[(2S)-4-(5-fluoropyrimidin-2-yl)-2-(trifluoromethyl)piperazin-1-yl]-1-(4-isopropylsulfonylphenyl)sulfonyl-indazole (24.6 mg, 35.7 μmol, 28.90% yield, 93.98% purity; Rt=1.730 minutes; E.E. by chiral HPLC (%)=100.0%) was isolated as a white solid) was isolated as a white solid. ¹H NMR (DMSO-d₆) δ 8.52 (s, 2H), 8.06-8.17 (m, 3H), 7.97-8.03 (m, 2H), 7.71 (m, 1H), 7.55 (d, 1H), 4.86 (br d, 1H), 4.67-4.79 (m, 1H), 4.38-4.46 (m, 1H), 3.74-3.85 (m, 1H), 3.63-3.71 (m, 1H), 3.53 (br s, 1H), 3.44 (br d, 1H), 3.09-3.20 (m, 1H), 1.05 (s, 6H) HPLC: 93.98% (220 nm), 92.60% (215 nm), 100.0% (254 nm). MS (ESI): mass calcd. For C₂₅H₂₃N₆S₂O₄ClF₄ 646.08 mass found 647.0 [M+H]⁺.

### Compound 70: 1-[(3R)-4-[4-chloro-1-(4-isopropylsulfonylphenyl)sulfonyl-indazol-3-yl]-3-(trifluoromethyl)piperazin-1-yl]-2,2,2-trifluoro-ethanone

### Compound 71: 1-[(3S)-4-[4-chloro-1-(4-isopropylsulfonylphenyl)sulfonyl-indazol-3-yl]-3-(trifluoromethyl)piperazin-1-yl]-2,2,2-trifluoro-ethanone

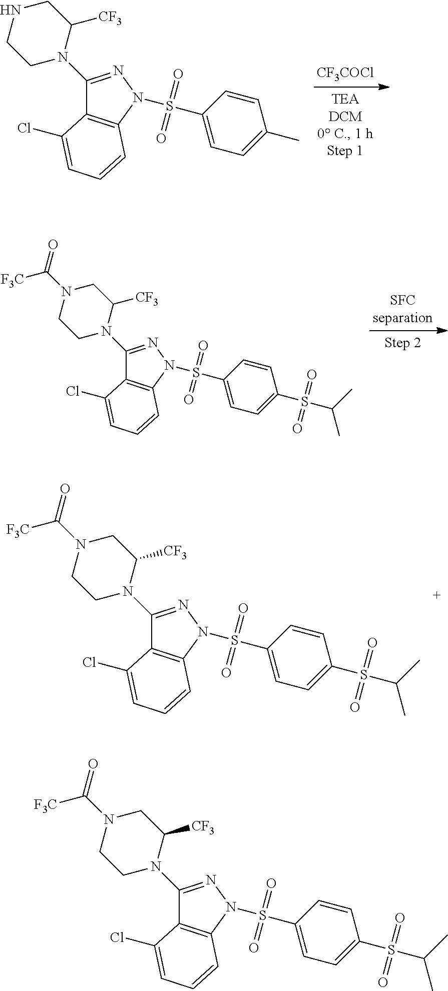 [SMILES: Cc1ccc(S(=O)(=O)n2nc(N3CCNCC3C(F)(F)F)c3c(Cl)cccc32)cc1 ; "CF3COCl" ; CC(C)S(=O)(=O)c1ccc(S(=O)(=O)n2nc(N3CCN(C(=O)C(F)(F)F)CC3C(F)(F)F)c3c(Cl)cccc32)cc1 ; CC(C)S(=O)(=O)c1ccc(S(=O)(=O)n2nc(N3CCN(C(=O)C(F)(F)F)C[C@@H]3C(F)(F)F)c3c(Cl)cccc32)cc1 ; CC(C)S(=O)(=O)c1ccc(S(=O)(=O)n2nc(N3CCN(C(=O)C(F)(F)F)C[C@H]3C(F)(F)F)c3c(Cl)cccc32)cc1]

Step 1: To a solution of 4-chloro-1-(4-isopropylsulfonylphenyl) sulfonyl-3-[2-(trifluoromethyl) piperazin-1-yl]indazole (67 mg, 122 μmol, 1 eq) in DCM (2 mL) was added TEA (123 mg, 1.22 mmol, 169 μL, 10 eq) and (2, 2, 2-trifluoroacetyl) 2, 2, 2-trifluoroacetate (255 mg, 1.22 mmol, 169 μL, 10 eq). The mixture was stirred at 0° C. for 1 hour. LC-MS showed 4-chloro-1-(4-isopropylsulfonylphenyl) sulfonyl-3-[2-(trifluoromethyl) piperazin-1-yl]indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-TLC (Silica gel, petroleum ether/ethyl acetate=2/1) to give desired 1-[4-[4-chloro-1-(4-isopropylsulfonylphenyl) sulfonyl-indazol-3-yl]-3-(trifluoromethyl) piperazin-1-yl]-2, 2, 2-trifluoro-ethanone (40 mg, 61.8 μmol, 50.8% yield) as a white solid. MS (ESI): mass calcd. For C₂₃H₂₁ClF₆N₄O₅S₂ 646.1 mass found 647.1 [M+H]⁺.

Step 2: 1-[4-[4-Chloro-1-(4-isopropylsulfonylphenyl)sulfonyl-indazol-3-yl]-3-(trifluoromethyl)piperazin-1-yl]-2,2,2-trifluoro-ethanone (40 mg, 61.8 μmol, 1 eq) was separated by chiral SFC (column: REGIS(S,S)WHELK-O1(250 mm*25 mm, 10 μm); mobile phase: [0.1% NH₃H₂O EtOH]; B %: 40%-40%, 10 min) to give two isomers. The structures were assigned randomly. 1-[(3R)-4-[4-Chloro-1-(4-isopropylsulfonylphenyl)sulfonyl-indazol-3-yl]-3-(trifluoromethyl)piperazin-1-yl]-2,2,2-trifluoro-ethanone (8 mg, 12.1 μmol, 19.6% yield, 98.10% purity; Rt=1.411 minutes; E.E. by chiral HPLC (%)=100.0%) was isolated as a white solid. ¹H NMR (400 MHz, DMSO-d₆) δ 8.15-8.09 (m, 3H), 8.03 (d, J=8.5 Hz, 2H), 7.71 (t, J=8.1 Hz, 1H), 7.55 (d, J=7.6 Hz, 1H), 4.86-4.72 (m, 1H), 4.30-4.16 (m, 1H), 4.12-3.90 (m, 1H), 3.69 (br dd, J=3.8, 14.8 Hz, 1H), 3.64-3.55 (m, 2H), 3.51 (br dd, J=3.0, 6.0 Hz, 1H), 3.46 (br d, J=6.8 Hz, 1H), 1.08 (d, J=6.9 Hz, 6H). HPLC: 98.10% (220 nm), 95.71% (215 nm), 99.45% (254 nm). MS (ESI): mass calcd. For C₂₃H₂₁ClF₆N₄O₅S₂ 646.1 mass found 647.1 [M+H]⁺. 1-[(3S)-4-[4-chloro-1-(4-isopropylsulfonylphenyl)sulfonyl-indazol-3-yl]-3-(trifluoromethyl)piperazin-1-yl]-2,2,2-trifluoro-ethanone (7.6 mg, 11.5 μmol, 18.7% yield, 98.24% purity; Rt=1.536 minutes; E.E. by chiral HPLC (%)=100.0%) was isolated as a white solid. ¹H NMR (400 MHz, DMSO-d₆) δ 8.16-8.08 (m, 3H), 8.03 (d, J=8.5 Hz, 2H), 7.74-7.68 (m, 1H), 7.55 (d, J=7.6 Hz, 1H), 4.78 (br d, J=4.0 Hz, 1H), 4.27-4.18 (m, 1H), 3.92 (br s, 1H), 3.74-3.64 (m, 1H), 3.64-3.55 (m, 2H), 3.51 (br d, J=6.0 Hz, 1H), 3.47 (br s, 1H), 1.08 (d, J=6.8 Hz, 6H). HPLC: 98.24% (220 nm), 96.03% (215 nm), 99.26% (254 nm). MS (ESI): mass calcd. For C₂₃H₂₁ClF₆N₄O₅S₂ 646.1 mass found 647.1 [M+H]⁺.

### Compound 72: 4-fluoro-3-[(2R)-4-(5-fluoropyrimidin-2-yl)-2-(trifluoromethyl)piperazin-1-yl]-1-(4-isopropylsulfonylphenyl)sulfonyl-indazole

### Compound 73: 4-fluoro-3-[(2S)-4-(5-fluoropyrimidin-2-yl)-2-(trifluoromethyl)piperazin-1-yl]-1-(4-isopropylsulfonylphenyl)sulfonyl-indazole

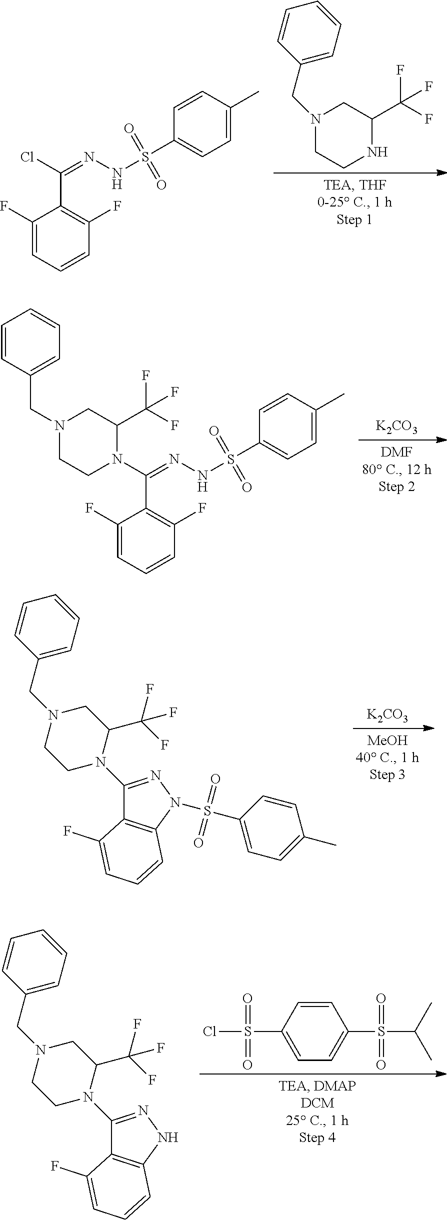

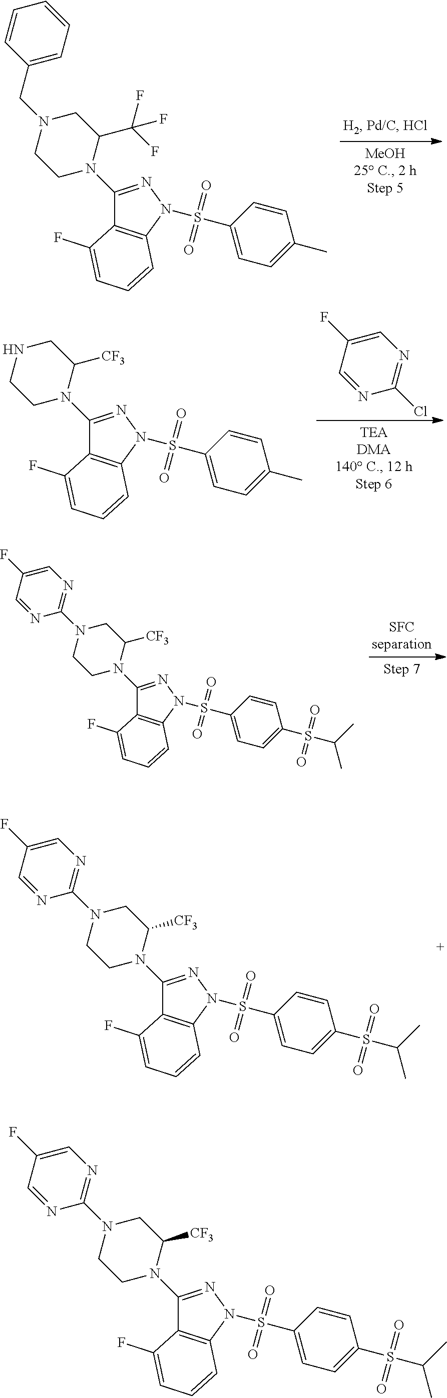 [SMILES: Cc1ccc(S(=O)(=O)n2nc(N3CCN(Cc4ccccc4)CC3C(F)(F)F)c3c(F)cccc32)cc1 ; Cc1ccc(S(=O)(=O)n2nc(N3CCNCC3C(F)(F)F)c3c(F)cccc32)cc1 ; Fc1cnc(Cl)nc1 ; CC(C)S(=O)(=O)c1ccc(S(=O)(=O)n2nc(N3CCN(c4ncc(F)cn4)CC3C(F)(F)F)c3c(F)cccc32)cc1 ; CC(C)S(=O)(=O)c1ccc(S(=O)(=O)n2nc(N3CCN(c4ncc(F)cn4)C[C@@H]3C(F)(F)F)c3c(F)cccc32)cc1 ; CC(C)S(=O)(=O)c1ccc(S(=O)(=O)n2nc(N3CCN(c4ncc(F)cn4)C[C@H]3C(F)(F)F)c3c(F)cccc32)cc1]

Step 1: To a solution of (1E)-2,6-difluoro-N-(p-tolylsulfonyl)benzohydrazonoyl chloride (847 mg, 2.46 mmol, 2 eq) and 1-benzyl-3-(trifluoromethyl) piperazine (300 mg, 1.23 mmol, 1 eq) in THE (10 mL) was added TEA (621 mg, 6.14 mmol, 855 μL, 5 eq) at 0′C. The mixture was stirred at 25° C. for 1 hour. LC-MS showed 1-benzyl-3-(trifluoromethyl) piperazine was consumed completely and desired mass was detected. The reaction mixture was partitioned between 20 mL of H₂O and 20 mL of EtOAc. The organic phase was separated, washed with 50 mL of brine, dried over Na₂SO₄, filtered and concentrated under reduced pressure to give desired N-[(E)-[[4-benzyl-2-(trifluoromethyl)piperazin-1-yl]-(2,6-difluorophenyl) methylene]amino]-4-methyl-benzenesulfonamide (1.13 g, crude) as a yellow oil. MS (ESI): mass calcd. For C₂₆H₂₅F₅N₄O₂S 552.1 mass found 553.1 [M+H]⁺.

Step 2: To a solution of 2N-[(E)-[[4-benzyl-2-(trifluoromethyl) piperazin-1-yl]-(2, 6-difluorophenyl) methylene]amino]-4-methyl-benzenesulfonamide (1.13 g, 2.05 mmol, 1 eq) in DMF (12 mL) was added K₂CO₃ (1.41 g, 10.2 mmol, 5 eq). The mixture was stirred at 80° C. for 12 hours. LC-MS showed N—[(F)-[[4-benzyl-2-(trifluoromethyl) piperazin-1-yl]-(2, 6-difluorophenyl) methylene]amino]-4-methyl-benzenesulfonamide was consumed completely and desired mass was detected. The reaction mixture was partitioned between 20 mL. of H₂O and 40 mL. of EtOAc. The organic phase was separated, washed with 50 mL. of brine, dried over Na₂SO₄, filtered and concentrated under reduced pressure to give desired 3-[4-benzyl-2-(trifluoromethyl)piperazin-1-yl]-4-fluoro-1-(p-tolylsulfonyl) indazole (1.1 g, crude) as a yellow oil. MS (ESI): mass calcd. For C₂₆H₁₂₄F₄N₄O₂S 532.1 mass found 533.1 [M+H]+

Step 3: To a solution of 3-[4-benzyl-2-(trifluoromethyl) piperazin-1-yl]-4-fluoro-1-(p-tolylsulfonyl) indazole (1.1 g, 2.07 mmol, 1 eq) in MeOH (11 mL) was added K₂CO₃ (1.43 g, 10.3 mmol, 5 eq). The mixture was stirred at 40° C. for 1 hour. LC-MS showed 3-[4-benzyl-2-(trifluoromethyl) piperazin-1-yl]-4-fluoro-1-(p-tolylsulfonyl) indazole was consumed completely and desired mass was detected. The reaction mixture was filtered and the filter liquor was concentrated under reduced pressure to give a residue. The residue was purified by prep-TLC (Silica gel, petroleum ether/ethyl acetate=2/1) to give desired 3-[4-benzyl-2-(trifluoromethyl)piperazin-1-yl]-4-fluoro-1H-indazole (320 mg, 846 μmol, 41.0% yield) as a yellow oil. MS (ESI): mass calcd. For C₁₉H₁₈F₄N₄ 378.1, mass found 379.1 [M+H]⁺.

Step 4: To a solution of 3-[4-benzyl-2-(trifluoromethyl)piperazin-1-yl]-4-fluoro-1H-indazole (320 mg, 846 μmol, 1 eq) and 4-isopropylsulfonylbenzenesulfonyl chloride (359 mg, 1.27 mmol, 1.5 eq) in DCM (5 mL) was added TEA (171 mg, 1.69 mmol, 235 μL, 2 eq) and DMAP (10.3 mg, 84.6 μmol, 0.1 eq). The mixture was stirred at 25° C. for 1 hour. LC-MS showed 3-[4-benzyl-2-(trifluoromethyl) piperazin-1-yl]-4-fluoro-1H-indazole was consumed completely and desired mass was detected. The reaction mixture was partitioned between 20 mL of H₂O and 30 mL of DCM. The organic phase was separated, washed with 50 mL of brine, dried over Na₂SO₄, filtered and concentrated under reduced pressure to give the crude product. The residue was purified by prep-TLC (Silica gel, petroleum ether/ethyl acetate=2/1) to give desired 3-[4-benzyl-2-(trifluoromethyl) piperazin-1-yl]-4-fluoro-1-(4-isopropylsulfonylphenyl) sulfonyl-indazole (210 mg, 336 μmol, 39.8% yield) as a yellow oil. MS (ESI): mass calcd. For C₂₆H₂₄F₄N₄O₂S 532.1, mass found 533.1 [M+H]⁺.

Step 5: A mixture of 3-[4-benzyl-2-(trifluoromethyl)piperazin-1-yl]-4-fluoro-1-(4-isopropylsulfonylphenyl)sulfonyl-indazole (130 mg, 208 μmol, 1 eq), Pd/C (13 mg, 10% purity), HCl (12 M, 34.7 μL, 2 eq) in MeOH (1 mL) was degassed and purged with H₂ (50 psi) for 3 times, and then the mixture was stirred at 25° C. for 2 hours under H₂ atmosphere. LC-MS showed 3-[4-benzyl-2-(trifluoromethyl) piperazin-1-yl]-4-fluoro-1-(4-isopropylsulfonylphenyl) sulfonyl-indazole was consumed completely and desired mass was detected. The reaction mixture was filtered and the filter cake was concentrated under reduced pressure to give desired 4-fluoro-1-(4-isopropylsulfonylphenyl) sulfonyl-3-[2-(trifluoromethyl) piperazin-1-yl]indazole (110 mg, crude) as a yellow solid. MS (ESI): mass calcd. For C₁₉H₁₈F₄N₄O₂S 442.1, mass found 443.1 [M+H]⁺.

Step 6: To a solution of 4-fluoro-1-(4-isopropylsulfonylphenyl) sulfonyl-3-[2-(trifluoromethyl) piperazin-1-yl]indazole (170 mg, 318 μmol, 1 eq) and 2-chloro-5-fluoro-pyrimidine (63.2 mg, 477 μmol, 1.5 eq) in NMP (1 mL) was added TEA (129 mg, 1.27 mmol, 177 μL, 4 eq). The mixture was stirred at 140° C. for 12 hours. LC-MS showed 4-fluoro-1-(4-isopropylsulfonylphenyl) sulfonyl-3-[2-(trifluoromethyl) piperazin-1-yl]indazole was consumed completely and desired mass was detected. The reaction mixture was partitioned between 5 mL of H₂O and 5 mL of DCM. The organic phase was separated, washed with 50 mL of brine, dried over Na₂SO₄, filtered and concentrated under reduced pressure to give the crude product. The residue was purified by prep-TLC (Silica gel, petroleum ether/ethyl acetate=3/1) to give desired 4-fluoro-3-[4-(5-fluoropyrimidin-2-yl)-2-(trifluoromethyl) piperazin-1-yl]-1-(4-isopropylsulfonylphenyl) sulfonyl-indazole (190 mg, 301 μmol, 20.2% yield) as a black oil. MS (ESI): mass calcd. For C₂₅H₂₃F₅N₆O₄S₂ 630.1 mass found 631.1 [M+H]⁺.

Step 7: 4-fluoro-3-[4-(5-fluoropyrimidin-2-yl)-2-(trifluoromethyl)piperazin-1-yl]-1-(4-isopropylsulfonylphenyl)sulfonyl-indazole (190 mg, 301 μmol, 1 eq) was separated by chrial SFC (column: REGIS(S,S)WHELK-O1 (250 mm*25 mm, 10 μm); mobile phase: [0.1% NH₃·H₂O MeOH]; B %: 42%-42%, 12 min) to give two isomers. The structures were assigned randomly. 4-Fluoro-3-[(2R)-4-(5-fluoropyrimidin-2-yl)-2-(trifluoromethyl)piperazin-1-yl]-1-(4-isopropylsulfonylphenyl)sulfonyl-indazole (16.7 mg, 26.1 μmol, 8.65% yield, 98.44% purity, Rt=1.471 minutes; E.E. by chiral HPLC (%)=99.18%) was isolated as a white solid. ¹H NMR (400 MHz, DMSO-d₆) δ 8.51 (s, 2H), 8.18-7.91 (m, 5H), 7.75 (dt, J=5.1, 8.2 Hz, 1H), 7.30 (dd, J=8.1, 11.3 Hz, 1H), 4.95 (br d, J=14.6 Hz, 1H), 4.78 (br d, J=5.4 Hz, 1H), 4.58 (br d, J=13.4 Hz, 1H), 3.80 (br s, 2H), 3.59-3.58 (m, 1H), 3.23-3.12 (m, 2H), 1.06 (d, J=6.8 Hz, 6H). HPLC: 98.44% (220 nm), 79.79% (215 nm), 97.63% (254 nm). MS (ESI): mass calcd. For C₂₅H₂₃F₅N₆O₄S₂ 630.1 mass found 631.1 [M+H]⁺. 1-[(3S)-4-[4-chloro-1-(4-isopropylsulfonylphenyl)sulfonyl-indazol-3-yl]-3-(trifluoromethyl)piperazin-1-yl]-2,2,2-trifluoro-ethanone (19.9 mg, 31.1 μmol, 10.3% yield, 96.24% purity, Rt=1.547 minutes; E.E. by chiral HPLC (%)=98.62%) was isolated as a white solid. ¹H NMR (400 MHz, DMSO-d₆) δ 8.51 (s, 2H), 8.09-7.96 (m, 5H), 7.75 (dt, J=5.1, 8.3 Hz, 1H), 7.30 (dd, J=8.1, 11.3 Hz, 1H), 4.95 (br d, J=14.6 Hz, 1H), 4.83-4.72 (m, 1H), 4.57 (br d, J=13.5 Hz, 1H), 3.78 (br d, J=12.4 Hz, 1H), 3.60-3.48 (m, 2H), 3.47-3.41 (m, 1H), 3.16 (br dd, J=9.4, 12.3 Hz, 1H), 1.05 (d, J=6.8 Hz, 6H). HPLC: 96.24% (220 nm), 93.62% (215 nm), 93.78% (254 nm). MS (ESI): mass calcd. For C₂₅H₂₃F₅N₆O₄S₂ 630.1 mass found 631.1 [M+H]⁺.

### Compound 74: 1-[4-[4-chloro-1-(4-isopropylsulfonylphenyl) sulfonyl-indazol-3-yl]-3-(trifluoromethyl) piperazin-1-yl]-2, 2-difluoro-ethanone

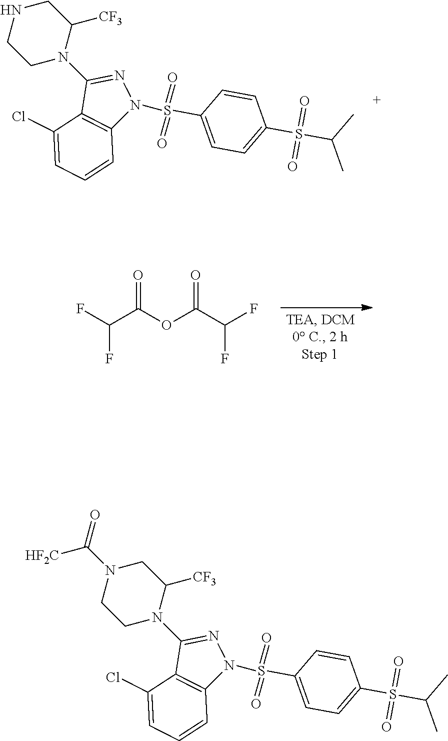

Step 1: To a solution of 4-chloro-1-(4-isopropylsulfonylphenyl) sulfonyl-3-[2-(trifluoromethyl) piperazin-1-yl]indazole (213 mg, 387 μmol, 1 eq) in DCM (3 mL) was added (2, 2-difluoroacetyl) 2, 2-difluoroacetate (674 mg, 3.87 mmol, 10 eq) and TEA (392 mg, 3.87 mmol, 539 μL, 10 eq). The mixture was stirred at 0° C. for 2 hours. LC-MS showed 4-chloro-1-(4-isopropylsulfonylphenyl) sulfonyl-3-[2-(trifluoromethyl) piperazin-1-yl]indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-HPLC (TFA condition; according to LCMS; Method: column: Phenomenex Luna C18 75*30 mm*3 m; mobile phase: [water(TFA)-ACN]; B %: 45%-75%, 8 min) to give desired 1-[4-[4-chloro-1-(4-isopropylsulfonylphenyl) sulfonyl-indazol-3-yl]-3-(trifluoromethyl) piperazin-1-yl]-2, 2-difluoro-ethanone (63.5 mg, 101 μmol, 26.0% yield, 99.88% purity) as a white solid. ¹H NMR (400 MHz, DMSO-d₆) δ 8.12 (td, J=4.4, 8.6 Hz, 3H), 8.02 (br d, J=8.6 Hz, 2H), 7.74-7.68 (m, 1H), 7.55 (d, J=7.8 Hz, 1H), 6.95-6.61 (m, 1H), 4.84-4.67 (m, 1H), 4.67-4.59 (m, 1H), 3.96 (br d, J=14.0 Hz, 1H), 3.66-3.54 (m, 2H), 3.53-3.49 (m, 1H), 3.49-3.44 (m, 2H), 1.08 (br d, J=6.8 Hz, 6H). HPLC: 99.88% (220 nm), 99.90% (215 nm), 100.00% (254 nm). MS (ESI): mass calcd. For C₂₃H₂₂ClF₅N₄O₅S₂ 628.1 mass found 629.1 [M+H]⁺.

### Compound 75: 4-chloro-1-(4-isopropylsulfonylphenyl)sulfonyl-3-[4-(2,2,2-trifluoroethyl)-2-(trifluoromethyl)piperazin-1-yl]indazole

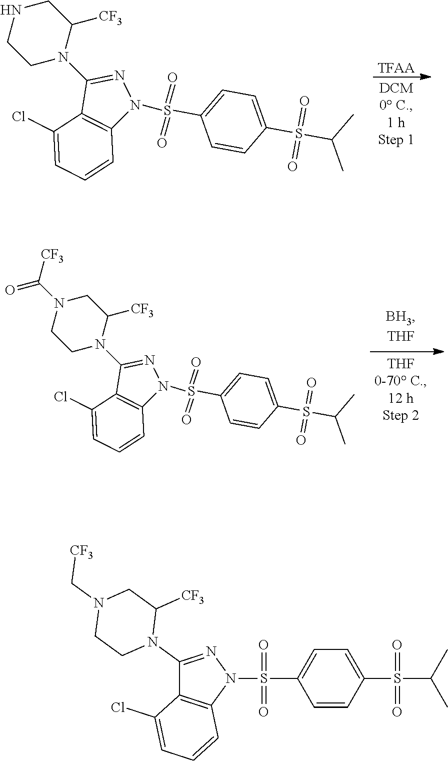 [SMILES: CC(C)S(=O)(=O)c1ccc(S(=O)(=O)n2nc(N3CCNCC3C(F)(F)F)c3c(Cl)cccc32)cc1 ; "TFAA" ; CC(C)S(=O)(=O)c1ccc(S(=O)(=O)n2nc(N3CCN(C(=O)C(F)(F)F)CC3C(F)(F)F)c3c(Cl)cccc32)cc1 ; CC(C)S(=O)(=O)c1ccc(S(=O)(=O)n2nc(N3CCN(CC(F)(F)F)CC3C(F)(F)F)c3c(Cl)cccc32)cc1]

Step 1: To a solution of 4-chloro-1-(4-isopropylsulfonylphenyl)sulfonyl-3-[2-(trifluoromethyl)piperazin-1-yl]indazole (200 mg, 362 μmol, 1 eq) in DCM (5.00 mL) was added TEA (183 mg, 1.81 mmol, 252 μL, 5 eq) and (2,2,2-trifluoroacetyl) 2,2,2-trifluoroacetate (228 mg, 1.09 mmol, 151 μL, 3 eq). The mixture was stirred at 0° C. for 1 hour. LC-MS showed 4-chloro-1-(4-isopropylsulfonylphenyl) sulfonyl-3-[2-(trifluoromethyl) piperazin-1-yl]indazole was consumed completely and one main peak with desired mass was detected. The reaction mixture was added to water (30 mL), extracted with EtOAc (30 mL*3). The combined organic layers were washed with brine (20 mL) and dried over Na₂SO₄, filtered and concentrated under reduced pressure to give a residue. The residue was purified by prep-TLC (Silica gel, petroleum ether/ethyl acetate=2/1) to give desired 1-[4-[4-chloro-1-(4-isopropylsulfonylphenyl) sulfonyl-indazol-3-yl]-3-(trifluoromethyl) piperazin-1-yl]-2, 2, 2-trifluoro-ethanone (200 mg, 309 μmol, 85.2% yield) as a white oil. MS (ESI): mass calcd. For C₂₃H₂₁S₂O₅N₄ClF₆ 646.05 mass found 647.0 [M+H]⁺.

Step 2: To a solution of 1-[4-[4-chloro-1-(4-isopropylsulfonylphenyl)sulfonyl-indazol-3-yl]-3-(trifluoromethyl)piperazin-1-yl]-2,2,2-trifluoro-ethanone (100 mg, 154 μmol, 1 eq) in THF (2.00 mL) was added BH₃·THF (1 M, 2.01 mL, 13 eq) at 0° C. The mixture was stirred at 70° C. for 12 hours. LC-MS showed 1-[4-[4-chloro-1-(4-isopropylsulfonylphenyl) sulfonyl-indazol-3-yl]-3-(trifluoromethyl) piperazin-1-yl]-2, 2, 2-trifluoro-ethenones consumed completely and one main peak with desired mass was detected. The reaction mixture was quenched by addition MeOH (10 mL) at 0° C., and stirred at 0° C. for 30 min, and then concentrated under reduced pressure to give a residue. The residue was purified by prep-HPLC (TFA condition:column: Phenomenex Luna C18 150*30 mm*5 μm; mobile phase: [water(TFA)-ACN]; B %: 45%-80%, 8 min) to give desired 4-chloro-1-(4-isopropylsulfonylphenyl)sulfonyl-3-[4-(2,2,2-trifluoroethyl)-2-(trifluoromethyl)piperazin-1-yl]indazole (20.0 mg, 31.6 μmol, 20.4% yield, 100.0% purity) as a white solid. MS (ESI): mass calcd. ¹H NMR (400 MHz, DMSO-d₆) δ 8.14-8.00 (m, 5H), 7.73-7.66 (m, 1H), 7.56-7.51 (m, 1H), 4.56-4.44 (m, 1H), 3.72 (s, 1H), 3.52-3.42 (m, 2H), 3.29-3.22 (m, 4H), 3.00-2.88 (m, 2H), 1.11-1.06 (m, 6H). HPLC: 99.46% (220 nm), 99.50% (215 nm), 100.0% (254 nm). MS (ESI): mass calcd. For C₂₃H₂₃S₂O₄N₄ClF₆ 632.08 mass found 633.3 [M+H]⁺.

### Compound 76: 1-[4-[4-chloro-1-[4-(difluoromethyl) phenyl]sulfonyl-indazol-3-yl]-3-(trifluoromethyl) piperazin-1-yl]-2, 2, 2-trifluoro-ethanone

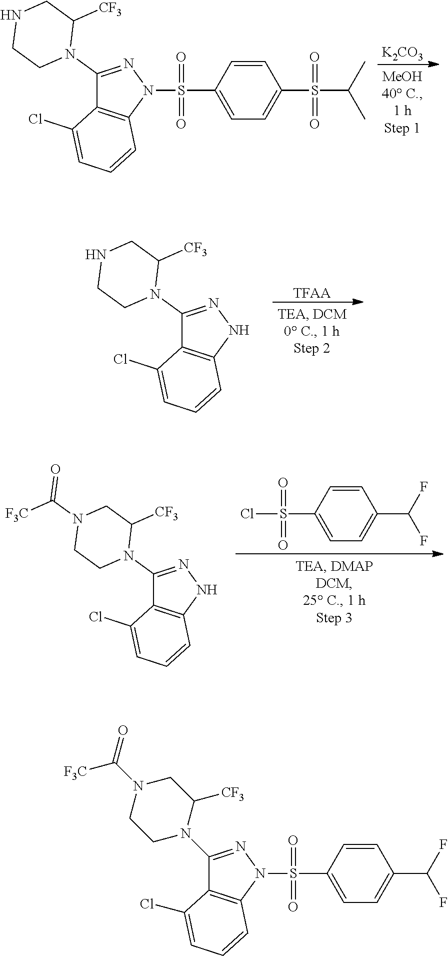

Step 1: To a solution of 4-chloro-1-(4-isopropylsulfonylphenyl) sulfonyl-3-[2-(trifluoromethyl) piperazin-1-yl]indazole (200 mg, 363 μmol, 1 eq) in MeOH (2 mL) was added K₂CO₃ (250 mg, 1.81 mmol, 50.5 μL, 5 eq). The mixture was stirred at 40° C. for 1 hour. LC-MS showed 4-chloro-1-(4-isopropylsulfonylphenyl) sulfonyl-3-[2-(trifluoromethyl) piperazin-1-yl]indazole was consumed completely and desired mass was detected. The reaction mixture was filtered and the filter liquor was concentrated under reduced pressure to give a residue and it was partitioned between 10 mL of H₂O and 10 mL of EtOAc. The organic phase was separated, washed with 50 mL of brine, dried over Na₂SO₄, filtered and concentrated under reduced pressure to give desired 4-chloro-3-[2-(trifluoromethyl) piperazin-1-yl]-1H-indazole (100 mg, crude) as a white solid. MS (ESI): mass calcd. For C₁₂H₁₂N₄F₃Cl 304.07 mass found 305.0 [M+H]⁺.

Step 2: To a solution of 4-chloro-3-[2-(trifluoromethyl)piperazin-1-yl]-1H-indazole (100 mg, 328.19 μmol, 1 eq) in DCM (2 mL) was added TEA (166 mg, 1.64 mmol, 228 μL, 5 eq) and (2, 2, 2-trifluoroacetyl) 2, 2, 2-trifluoroacetate (206 mg, 984 μmol, 136 μL, 3 eq). The mixture was stirred at 0° C. for 1 hour. LC-MS showed 4-chloro-3-[2-(trifluoromethyl) piperazin-1-yl]-1H-indazole was consumed completely and desired mass was detected. The reaction mixture was partitioned between 30 mL of H₂O and 30 mL of EtOAc. The organic phase was separated, washed with 50 mL of brine, dried over Na₂SO₄, filtered and concentrated under reduced pressure to give the residue. The residue was purified by prep-TLC (Silica gel, petroleum ether/ethyl acetate=2/1) to give desired 1-[4-(4-chloro-1H-indazol-3-yl)-3-(trifluoromethyl) piperazin-1-yl]-2, 2, 2-trifluoro-ethanone (45 mg, 112 μmol, 34.22% yield) as a white oil. MS (ESI): mass calcd. For C₁₄H₁₁N₄ClOF₆ 400.5 mass found 401.0 [M+H]⁺.

Step 3: To a solution of 1-[4-(4-chloro-1H-indazol-3-yl)-3-(trifluoromethyl)piperazin-1-yl]-2,2,2-trifluoro-ethanone (20 mg, 49.9 μmol, 1 eq) and 4-(difluoromethyl)benzenesulfonyl chloride (45.2 mg, 199 μmol, 4 eq) in DCM (1 mL) was added TEA (10.1 mg, 99.8 μmol, 13.9 μL, 2 eq) and DMAP (609 pg, 4.99 μmol, 0.1 eq). The mixture was stirred at 25° C. for 1 hour. LC-MS showed 1-[4-(4-chloro-1H-indazol-3-yl)-3-(trifluoromethyl) piperazin-1-yl]-2, 2, 2-trifluoro-ethanone was consumed completely and desired mass was detected. The residue was purified by prep-HPLC (TFA condition; Method:column: Phenomenex Luna C18 75*30 mm*3 μm; mobile phase: [water(TFA)-ACN]; B %: 55%-85%, 8 min) to give desired 1-[4-[4-chloro-1-[4-(difluoromethyl)phenyl]sulfonyl-indazol-3-yl]-3-(trifluoromethyl)piperazin-1-yl]-2,2,2-trifluoro-ethanone (4.1 mg, 6.92 μmol, 13.87% yield, 99.78% purity) as a white solid. ¹H NMR (400 MHz, DMSO-d₆) δ 8.12 (d, J=8.0 Hz, 1H), 8.03 (br d, J=8.3 Hz, 2H), 7.76 (d, J=8.3 Hz, 2H), 7.70 (t, J=8.1 Hz, 1H), 7.54 (d, J=7.9 Hz, 1H), 7.26-6.94 (m, 1H), 4.65 (br d, J=14.3 Hz, 1H), 4.27-4.19 (m, 2H), 3.96-3.91 (m, 2H), 3.73 (br d, J=1.6 Hz, 2H). HPLC: 99.78% (220 nm), 98.65% (215 nm), 100.00% (254 nm). MS (ESI): mass calcd. For C₂₁H₁₅N₄O₃F₈ClS 590.04 mass found 591.0 [M+H]⁺.

### Compound 77: 4-chloro-1-[4-(difluoromethyl)phenyl]sulfonyl-3-[4-(5-fluoropyrimidin-2-yl)-2-(trifluoromethyl)piperazin-1-yl]indazole

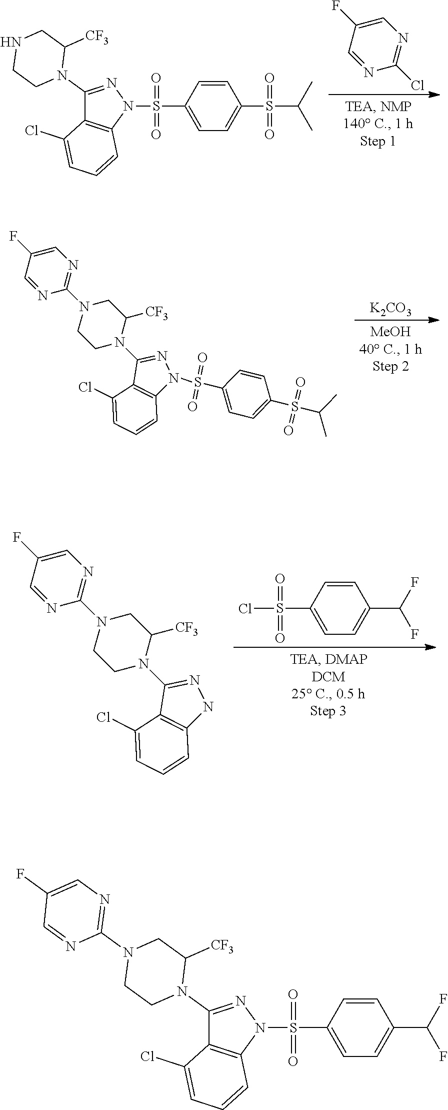

Step 1: To a solution of 4-chloro-1-(4-isopropylsulfonylphenyl)sulfonyl-3-[2-(trifluoromethyl)piperazin-1-yl]indazole (200 mg, 362 μmol, 1 eq) and 2-chloro-5-fluoro-pyrimidine (72.1 mg, 544 μmol, 67.4 μL, 1.5 eq) in NMP (3 mL) was added TEA (146 mg, 1.45 mmol, 202 μL, 4 eq). The mixture was stirred at 140° C. for 1 hour. LC-MS showed 4-chloro-1-(4-isopropylsulfonylphenyl) sulfonyl-3-[2-(trifluoromethyl) piperazin-1-yl]indazole was consumed completely and desired mass was detected. The reaction mixture was partitioned between 20 mL of H₂O and 20 mL of EtOAc. The organic phase was separated, washed with 50 mL of brine, dried over Na₂SO₄, filtered and concentrated under reduced pressure to give a residue. The residue was purified by prep-TLC (Silica gel, petroleum ether/ethyl acetate=2/1) to give desired 4-chloro-3-[4-(5-fluoropyrimidin-2-yl)-2-(trifluoromethyl) piperazin-1-yl]-1-(4-isopropylsulfonylphenyl) sulfonyl-indazole (140 mg, 216 μmol, 59.6% yield) as a yellow oil. MS (ESI): mass calcd. For C₂₅H₂₃N₆ClS₂O₄F₄ 646.08 mass found 647.0 [M+H]⁺.

Step 2: To a solution of 4-chloro-3-[4-(5-fluoropyrimidin-2-yl)-2-(trifluoromethyl) piperazin-1-yl]-1-(4-isopropylsulfonylphenyl) sulfonyl-indazole (140 mg, 216 μmol, 1 eq) in MeOH (2 mL) was added K₂CO₃ (149 mg, 1.08 mmol, 5 eq). The mixture was stirred at 40° C. for 1 hour. LC-MS showed 4-chloro-3-[4-(5-fluoropyrimidin-2-yl)-2-(trifluoromethyl) piperazin-1-yl]-1-(4-isopropylsulfonylphenyl) sulfonyl-indazole was consumed completely and desired mass was detected. The reaction mixture was filtered and the filter liquor was concentrated under reduced pressure to give a residue then it was partitioned between 10 mL of H₂O and 10 mL of EtOAc. The organic phase was separated, washed with 50 mL of brine, dried over Na₂SO₄, filtered and concentrated under reduced pressure to give desired 4-chloro-3-[4-(5-fluoropyrimidin-2-yl)-2-(trifluoromethyl)piperazin-1-yl]-1H-indazole (92 mg, crude) as a yellow oil. MS (ESI): mass calcd. For C₁₆H₁₃N₆F₄Cl 400.08 mass found 401.0 [M+H]⁺.

Step 3: To a solution of 4-chloro-3-[4-(5-fluoropyrimidin-2-yl)-2-(trifluoromethyl)piperazin-1-yl]-1H-indazole (20 mg, 49.9 μmol, 1 eq) and 4-(difluoromethyl) benzenesulfonyl chloride (45.2 mg, 199 μmol, 4 eq) in DCM (1 mL) was added TEA (10.1 mg, 99.8 μmol, 13.8 μL, 2 eq) and DMAP (609 pg, 4.99 μmol, 0.1 eq). The mixture was stirred at 25° C. for 0.5 hour. LC-MS showed 4-chloro-3-[4-(5-fluoropyrimidin-2-yl)-2-(trifluoromethyl) piperazin-1-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give a residue. The residue was purified by prep-HPLC (TFA condition; Method: column: Phenomenex Luna C18 75*30 mm*3 μm; mobile phase: [water(TFA)-ACN]; B %: 60%-90%, 8 min) to give desired 4-chloro-1-[4-(difluoromethyl)phenyl]sulfonyl-3-[4-(5-fluoropyrimidin-2-yl)-2-(trifluoromethyl)piperazin-1-yl]indazole (13.1 mg, 21.6 μmol, 43.36% yield, 97.60% purity) as a white solid. ¹H NMR (400 MHz, DMSO-d₆) δ 8.54 (s, 2H), 8.12 (br d, J=8.3 Hz, 1H), 8.00 (br d, J=7.9 Hz, 2H), 7.77-7.66 (m, 3H), 7.54 (d, J=7.5 Hz, 1H), 7.09 (s, 1H), 4.88 (br d, J=14.3 Hz, 1H), 4.42 (br d, J=12.4 Hz, 1H), 3.88-3.78 (m, 2H), 3.75-3.64 (m, 2H), 3.53 (br d, J=1.3 Hz, 1H). HPLC: 97.60% (220 nm), 97.17% (215 nm), 97.18% (254 nm). MS (ESI): mass calcd. For C₂₃H₁₇N₆SO₂ClF₆ 590.07, mass found 591.0 [M+H]⁺.

### Compound 78: 4-chloro-1-[4-(difluoromethyl) phenyl]sulfonyl-3-[7-(5-fluoropyrimidin-2-yl)-4, 7-diazaspiro [2.5]octan-4-yl]indazole

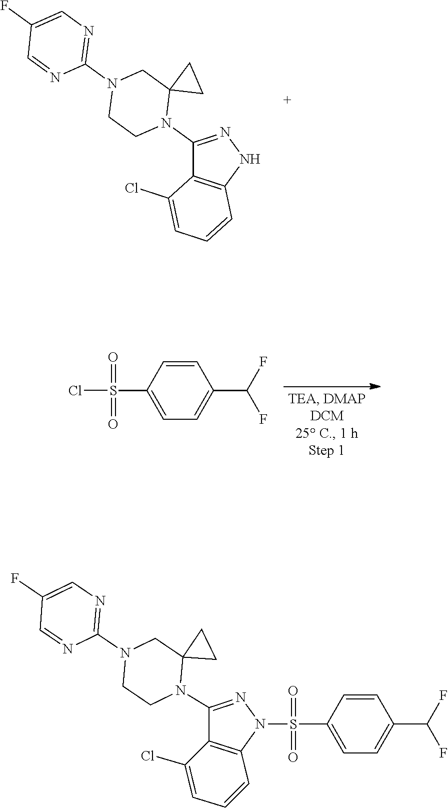 [SMILES: Fc1cnc(N2CCN(c3n[nH]c4cccc(Cl)c34)C3(CC3)C2)nc1 ; O=S(=O)(Cl)c1ccc(C(F)F)cc1 ; O=S(=O)(c1ccc(C(F)F)cc1)n1nc(N2CCN(c3ncc(F)cn3)CC23CC3)c2c(Cl)cccc21]

Step 1: To a solution of 4-chloro-3-[7-(5-fluoropyrimidin-2-yl)-4,7-diazaspiro[2.5]octan-4-yl]-1H-indazole (20 mg, 55.7 μmol, 1 eq) and 4-(difluoromethyl)benzenesulfonyl chloride (25.27 mg, 111 μmol, 2 eq) in DCM (1 mL) was added TEA (11.28 mg, 111 μmol, 15.5 μL, 2 eq) and DMAP (681 pg, 5.57 μmol, 0.1 eq). The mixture was stirred at 25° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-HPLC (TFA condition; according to LCMS; Method: column: Phenomenex Luna C18 75*30 mm*3 m; mobile phase: [water(TFA)-ACN]; B %: 55%-85%, 8 min to give desired 4-chloro-1-[4-(difluoromethyl) phenyl]sulfonyl-3-[7-(5-fluoropyrimidin-2-yl)-4, 7-diazaspiro [2.5]octan-4-yl]indazole (2.5 mg, 4.55 μmol, 8.15% yield, 99.81% purity) as a white solid. ¹H NMR (400 MHz, DMSO-d₆) δ 8.46 (s, 2H), 8.07 (d, J=8.4 Hz, 1H), 7.93 (d, J=8.3 Hz, 2H), 7.71 (d, J=8.3 Hz, 2H), 7.63 (s, 1H), 7.46 (d, J=7.7 Hz, 1H), 7.20-6.91 (m, 1H), 3.92 (s, 2H), 3.53 (br s, 2H), 1.25-1.21 (m, 2H), 0.78 (br s, 2H), 0.37 (br s, 2H). HPLC: 99.81% (220 nm), 99.93% (215 nm), 100.0% (254 nm). MS (ESI): mass calcd. For C₂₄H₂₀ClF₃N₆O₂S 548.1 mass found 549.1 [M+H]⁺.

### Compound 79: 4-chloro-3-[4-chloro-6-(trifluoromethyl)-3, 6-dihydro-2H-pyridin-1-yl]-1-(4-isopropylsulfonylphenyl) sulfonyl-indazole

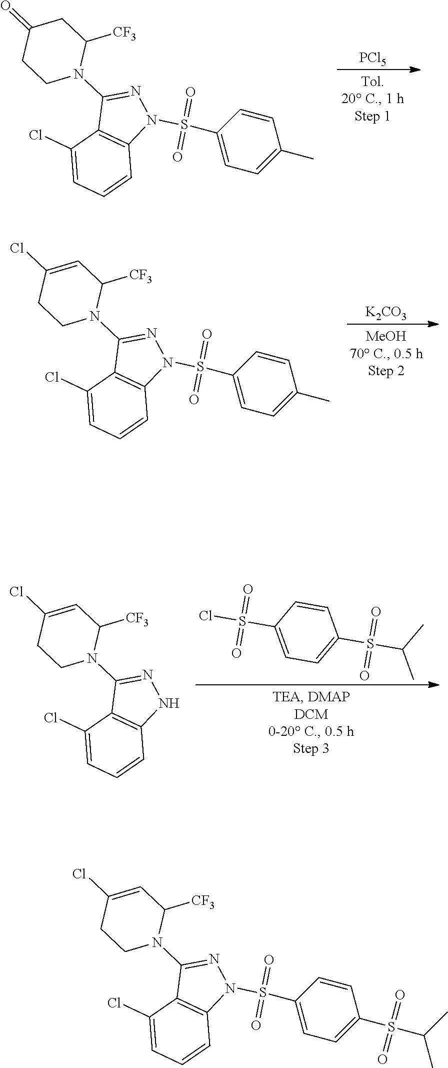

Step 1: To a solution of 1-[4-chloro-1-(p-tolylsulfonyl) indazol-3-yl]-2-(trifluoromethyl) piperidin-4-one (110 mg, 233 μmol, 1 eq) in Tol. (1 mL) was added PCl₅ (121 mg, 583 μmol 2.5 eq). The mixture was stirred at 20° C. for 1 hour. LC-MS showed 1-[4-chloro-α-(p-tolylsulfonyl) indazol-3-yl]-2-(trifluoromethyl) piperidin-4-one was consumed completely and desired mass was detected. The pH of the mixture was adjusted to 10-11 with 2N NaOH. The reaction mixture was added to water (20 mL), extracted with EtOAc (10 mL*3). The combined organic layers were washed with brine (20 mL) and dried over Na₂SO₄, filtered and concentrated under reduced pressure to give desired 4-chloro-3-[4-chloro-6-(trifluoromethyl)-3, 6-dihydro-2H-pyridin-1-yl]-1-(p-tolylsulfonyl) indazole (50 mg, crude) as a yellow solid. MS (ESI): mass calcd. For C₂₀H₁₆Cl₂F₃N₃O₂S 489.03, mass found 489.9 [M+H]⁺.

Step 2: To a solution of 4-chloro-3-[4-chloro-6-(trifluoromethyl)-3, 6-dihydro-2H-pyridin-1-yl]-1-(p-tolylsulfonyl) indazole (50 mg, 102 μmol, 1 eq) in MeOH (2 mL) was added K₂CO₃ (70.5 mg, 510 μmol, 5 eq). The mixture was stirred at 70° C. for 0.5 hour. TLC (petroleum ether/ethyl acetate=5/1) indicated 4-chloro-3-[4-chloro-6-(trifluoromethyl)-3, 6-dihydro-2H-pyridin-1-yl]-1-(p-tolylsulfonyl) indazole was consumed completely and one new spot formed. The reaction was clean according to TLC. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (Silica gel, petroleum ether/ethyl acetate=5/1) to give desired 4-chloro-3-[4-chloro-6-(trifluoromethyl)-3,6-dihydro-2H-pyridin-1-yl]-1H-indazole (20 mg, 59.5 μmol, 58.4% yield) as a yellow solid.

Step 3: To a solution of 4-isopropylsulfonylbenzenesulfonyl chloride (25.2 mg, 89.3 mol, 1.5 eq) and 4-chloro-3-[4-chloro-6-(trifluoromethyl)-3,6-dihydro-2H-pyridin-1-yl]-1H-indazole (20 mg, 59.5 μmol, 1 eq) in DCM (1 mL) was added TEA (12.0 mg, 119 μmol, 16.6 μL, 2 eq) and DMAP (727 pg, 5.95 μmol, 0.1 eq) at 0° C. The mixture was stirred at 20° C. for 0.5 hour. LC-MS showed 4-chloro-3-[4-chloro-6-(trifluoromethyl)-3, 6-dihydro-2H-pyridin-1-yl]-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water(NH₄HCO₃)-ACN]; B %: 60%-90%, 8 min) to give desired 4-chloro-3-[4-chloro-6-(trifluoromethyl)-3,6-dihydro-2H-pyridin-1-yl]-1-(4-isopropylsulfonylphenyl)sulfonyl-indazole (15.1 mg, 25.3 μmol, 42.5% yield, 97.61% purity) as a yellow solid. ¹H NMR (400 MHz, DMSO-d₆) δ 8.16-8.07 (m, 3H), 8.06-7.99 (m, 2H), 7.71 (t, J=8.1 Hz, 1H), 7.55 (d, J=7.8 Hz, 1H), 6.21-6.03 (m, 1H), 4.74 (br t, J=8.3 Hz, 1H), 4.26-4.04 (m, 1H), 3.90 (br d, J=2.3 Hz, 1H), 3.69-3.43 (m, 2H), 3.32-3.21 (m, 1H), 1.17-1.05 (m, 6H). HPLC: 97.61% (220 nm), 97.11% (210 nm), 98.82% (254 nm). MS (ESI): mass calcd. For C₂₂H₂₀Cl₂F₃N₃O₄S₂ 581.02, mass found 582.0 [M+H]⁺.

### Compound 80: 4-chloro-3-[(6R)-4-chloro-6-(trifluoromethyl)-3, 6-dihydro-2H-pyridin-1-yl]-1-(4-isopropylsulfonylphenyl)sulfonyl-indazole

### Compound 81: 4-chloro-3-[(6S)-4-chloro-6-(trifluoromethyl)-3,6-dihydro-2H-pyridin-1-yl]-1-(4-isopropylsulfonylphenyl)sulfonyl-indazole

### Compound 82: 4-chloro-3-[(2R)-4-chloro-2-(trifluoromethyl)-3,6-dihydro-2H-pyridin-1-yl]-1-(4-isopropylsulfonylphenyl)sulfonyl-indazole

### Compound 83: 4-chloro-3-[(2S)-4-chloro-2-(trifluoromethyl)-3,6-dihydro-2H-pyridin-1-yl]-1-(4-isopropylsulfonylphenyl)sulfonyl-indazole

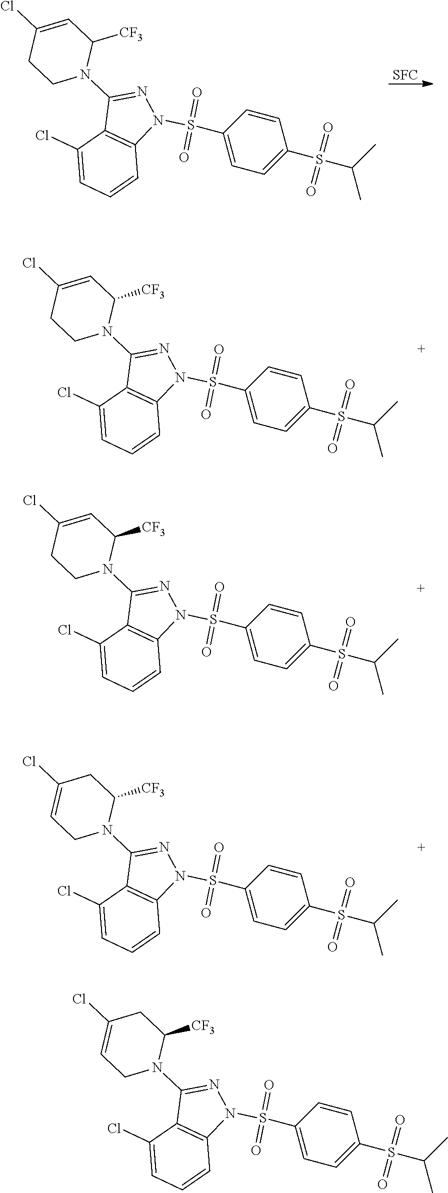 [SMILES: CC(C)S(=O)(=O)c1ccc(S(=O)(=O)n2nc(N3CCC(Cl)=CC3C(F)(F)F)c3c(Cl)cccc32)cc1 ; "SFC" ; CC(C)S(=O)(=O)c1ccc(S(=O)(=O)n2nc(N3CCC(Cl)=C[C@@H]3C(F)(F)F)c3c(Cl)cccc32)cc1 ; CC(C)S(=O)(=O)c1ccc(S(=O)(=O)n2nc(N3CCC(Cl)=C[C@H]3C(F)(F)F)c3c(Cl)cccc32)cc1 ; CC(C)S(=O)(=O)c1ccc(S(=O)(=O)n2nc(N3CC=C(Cl)C[C@@H]3C(F)(F)F)c3c(Cl)cccc32)cc1 ; CC(C)S(=O)(=O)c1ccc(S(=O)(=O)n2nc(N3CC=C(Cl)C[C@H]3C(F)(F)F)c3c(Cl)cccc32)cc1]

Step 1: The 4-chloro-3-[4-chloro-6-(trifluoromethyl)-3,6-dihydro-2H-pyridin-1-yl]-1-(4-isopropylsulfonylphenyl)sulfonyl-indazole (9 mg, 15.5 μmol) was separated by SFC (condition: column: DAICEL CHIRALPAK IC (250 mm*30 mm*10 μm); mobile phase: [0.1%₀NH₃H₂O EtOH]; B %: 15%-45%, 16 min) to give four isomers. The structures were assigned randomly. 4-Chloro-3-[(6R)-4-chloro-6-(trifluoromethyl)-3, 6-dihydro-2H-pyridin-1-yl]-1-(4-isopropylsulfonylphenyl)sulfonyl-indazole (1.3 mg, 2.06 μmol, 13.32% yield, 92.20% purity, Rt=1.876 minutes; E.E. by chiral HPLC (%)=100.0%) was isolated as a white solid. ¹H NMR (400 MHz, DMSO-d₆) δ 8.14-8.07 (m, 3H), 8.04-7.97 (m, 2H), 7.71 (t, J=8.1 Hz, 1H), 7.55 (d, J=7.5 Hz, 1H), 6.28-6.05 (m, 1H), 5.21-5.08 (m, 1H), 3.69-3.57 (m, 1H), 3.55-3.44 (m, 2H), 2.30-2.15 (m, 2H), 1.09 (dd, J=5.9, 6.5 Hz, 6H). MS (ESI): mass calcd. For C₂₂H₂₀Cl₂F₃N₃O₄S₂ 581.02, mass found 582.0 [M+H]⁺. 4-Chloro-3-[(6S)-4-chloro-6-(trifluoromethyl)-3,6-dihydro-2H-pyridin-1-yl]-1-(4-isopropylsulfonylphenyl)sulfonyl-indazole (0.9 mg, 1.45 μmol, 9.38% yield, 93.79% purity, Rt=1.976 minutes; E.E. by chiral HPLC (%)=100.0%) was isolated as a white solid. ¹H NMR (400 MHz, DMSO-d₆) δ 8.15-8.05 (m, 3H), 8.03-7.98 (m, 2H), 7.71 (t, J=8.2 Hz, 1H), 7.54 (d, J=7.6 Hz, 1H), 6.25-6.12 (m, 1H), 5.22-5.08 (m, 1H), 3.70-3.65 (m, 1H), 3.56-3.52 (m, 2H), 2.36-2.23 (m, 2H), 1.11-1.06 (m, 6H). MS (ESI): mass calcd. For C₂₂H₂OCl₂F₃N₃O₄S₂ 581.02, mass found 582.0 [M+H]⁺. 4-Chloro-3-[(2R)-4-chloro-2-(trifluoromethyl)-3,6-dihydro-2H-pyridin-1-yl]-1-(4-isopropylsulfonylphenyl)sulfonyl-indazole (2.4 mg, 4.02 μmol, 26.03% yield, 97.62% purity, Rt=2.183 minutes; E.E. by chiral HPLC (%)=100.0%) was isolated as a white solid. ¹H NMR (400 MHz, DMSO-d₆) δ 8.17-8.08 (m, 3H), 8.06-7.99 (m, 2H), 7.70 (t, J=8.1 Hz, 1H), 7.54 (d, J=7.8 Hz, 1H), 6.16-5.97 (m, 1H), 4.80-4.66 (m, 1H), 4.17-4.06 (m, 1H), 3.93-3.81 (m, 1H), 3.48 (br d, J=6.9 Hz, 1H), 3.27-3.21 (m, 1H), 2.49-2.42 (m, 1H), 1.12-1.06 (m, 6H). MS (ESI): mass calcd. For C₂₂H₂₀Cl₂F₃N₃O₄S₂ 581.02, mass found 582.0 [M+H]⁺. 4-Chloro-3-[(2S)-4-chloro-2-(trifluoromethyl)-3,6-dihydro-2H-pyridin-1-yl]-1-(4-isopropylsulfonylphenyl)sulfonyl-indazole (1.7 mg, 2.78 μmol, 18.00% yield, 95.30% purity, Rt=2.359 minutes; E.E. by chiral HPLC (%)=100.0%) was isolated as a white solid. ¹H NMR (400 MHz, DMSO-d₆) δ 8.14-8.08 (m, 3H), 8.06-8.00 (m, 2H), 7.70 (t, J=8.2 Hz, 1H), 7.54 (d, J=7.6 Hz, 1H), 6.20-5.98 (m, 1H), 4.73 (br t, J=8.3 Hz, 1H), 4.12 (br d, J=18.4 Hz, 1H), 3.87 (br dd, J=2.6, 17.1 Hz, 1H), 3.50-3.45 (m, 1H), 3.26-3.20 (m, 1H), 2.47-2.42 (m, 1H), 1.09 (dd, J=7.0, 7.9 Hz, 6H). HPLC: 97.61% (220 nm), 97.11% (210 nm), 98.82% (254 nm). MS (ESI): mass calcd. For C₂₂H₂₀Cl₂F₃N₃O₄S₂ 581.02, mass found 582.0 [M+H]f.

### Compound 84: 1-[4-chloro-1-(4-isopropylsulfonylphenyl)sulfonyl-indazol-3-yl]-2-(trifluoromethyl)piperidin-4-one

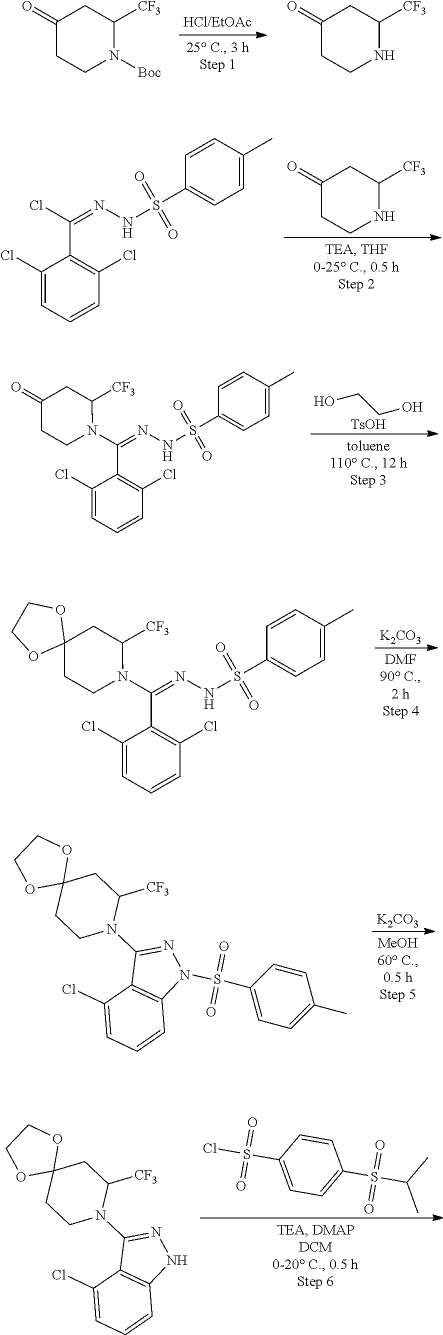

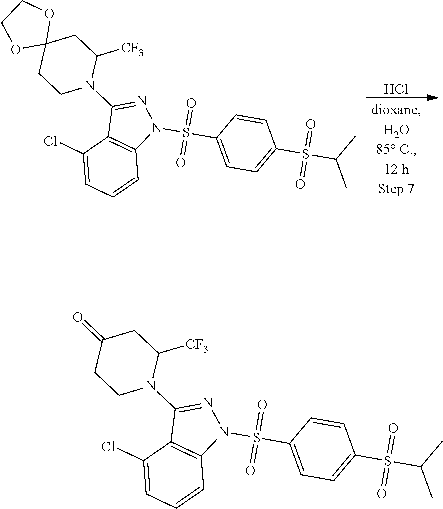 [SMILES: CC(C)S(=O)(=O)c1ccc(S(=O)(=O)n2nc(N3CCC4(CC3C(F)(F)F)OCCO4)c3c(Cl)cccc32)cc1 ; Cl ; CC(C)S(=O)(=O)c1ccc(S(=O)(=O)n2nc(N3CCC(=O)CC3C(F)(F)F)c3c(Cl)cccc32)cc1]

Step 1: A mixture of tert-butyl 4-oxo-2-(trifluoromethyl) piperidine-1-carboxylate (4.8 g, 18.0 mmol, 1 eq) in HCl/EtOAc (4M, 44.9 mL, 10 eq) was stirred at 25° C. for 3 hours. TLC (petroleum ether/ethyl acetate=3/1) showed tert-butyl 4-oxo-2-(trifluoromethyl) piperidine-1-carboxylate was consumed completely. The reaction was concentrated to give desired 2-(trifluoromethyl) piperidin-4-one (3.66 g, crude, HCl) as a white solid.

Step 2: To a solution of 2-(trifluoromethyl) piperidin-4-one (2.16 g, 10.6 mmol, 1 eq, HCl) in THE (3 mL) was added dropwise TEA (10.7 g, 106 mmol, 14.8 mL, 10 eq) at 25° C. After addition, the mixture was stirred at this temperature for 10 mins, and then (1Z)-2, 6-dichloro-N-(p-tolylsulfonyl) benzohydrazonoyl chloride (4.41 g, 11.7 mmol, 1.1 eq) in THF (3 mL) was added dropwise at 0° C. The resulting mixture was stirred at 25° C. for 20 mins. LC-MS showed 2-(trifluoromethyl) piperidin-4-one was consumed completely and one main peak with desired mass was detected. Then it was separated between 200 mL of water and 400 mL of ethyl acetate. The organic phase was separated, washed with 300 mL of brine, dried over Na₂SO₄, filtered and concentrated under reduced pressure to give the crude product. The residue was purified by column chromatography (Silica gel, petroleum ether/ethyl acetate=1/0 to 1/1) to give desired N-[(E)-[(2, 6-dichlorophenyl)-[4-oxo-2-(trifluoromethyl)-1-piperidyl]methylene]amino]-4-methyl-benzenesulfonamide (5 g, crude) as a yellow solid. MS (ESI): mass calcd. For C₂₀H₁₈Cl₂F₃N₃O₃S 507.04, mass found 508.0 [M+H]⁺.

Step 3: To a solution of N-[(E)-[(2, 6-dichlorophenyl)-[4-oxo-2-(trifluoromethyl)-1-piperidyl]methylene]amino]-4-methyl-benzenesulfonamide (0.5 g, 984 μmol, 1 eq) in Tol. (6 mL) was added TsOH·H₂O (18.7 mg, 98.4 μmol, 0.1 eq) and ethylene glycol (183 mg, 2.95 mmol, 165 μL, 3 eq). The mixture was stirred at 110° C. for 12 hours. LC-MS showed N-[(E)-[(2, 6-dichlorophenyl)-[4-oxo-2-(trifluoromethyl)-1-piperidyl]methylene]amino]-4-methyl-benzenesulfonamide was consumed completely and desired mass was detected. The reaction mixture was concentrated to give the crude product. The residue was purified by column chromatography (ISCO®; 20 g SepaFlash® Silica Flash Column, Eluent of 0˜8% Ethyl acetate/petroleum ether gradient @50 mL/min) to give desired N-[(E)-[(2, 6-dichlorophenyl)-[7-(trifluoromethyl)-1, 4-dioxa-8-azaspiro [4.5]decan-8-yl]methylene]amino]-4-methyl-benzenesulfonamide (540 mg, crude) as a yellow solid. MS (ESI): mass calcd. For C₂₂H₂₂Cl₂F₃N₃O₄S 551.07, mass found 552.0 [M+H]⁺.

Step 4: To a solution of N-[(E)-[(2, 6-dichlorophenyl)-[7-(trifluoromethyl)-1, 4-dioxa-8-azaspiro [4.5]decan-8-yl]methylene]amino]-4-methyl-benzenesulfonamide (540 mg, 978 μmol, 1 eq) in DMF (10 mL) was added K₂CO₃ (540 mg, 3.91 mmol, 4 eq). The mixture was stirred at 90° C. for 2 hours. TLC indicated N-[(E)-[(2, 6-dichlorophenyl)-[7-(trifluoromethyl)-1, 4-dioxa-8-azaspiro [4.5]decan-8-yl]methylene]amino]-4-methyl-benzenesulfonamide was consumed completely and one new spot formed. The reaction mixture was added to water (20 mL), extracted with EtOAc (10 mL*3). The combined organic layers were washed with brine (20 mL) and dried over Na₂SO₄, filtered and concentrated under reduced pressure to give desired 8-[4-chloro-1-(p-tolylsulfonyl) indazol-3-yl]-7-(trifluoromethyl)-1,4-dioxa-8-azaspiro[4.5]decane (500 mg, crude) as an orange oil.

Step 5: To a solution of 8-[4-chloro-1-(p-tolylsulfonyl) indazol-3-yl]-7-(trifluoromethyl)-1, 4-dioxa-8-azaspiro [4.5]decane (500 mg, 969 μmol, 1 eq) in MeOH (10 mL) was added K₂CO₃ (670 mg, 4.85 mmol, 5 eq). The mixture was stirred at 60° C. for 0.5 hour. LC-MS showed 8-[4-chloro-1-(p-tolylsulfonyl) indazol-3-yl]-7-(trifluoromethyl)-1, 4-dioxa-8-azaspiro [4.5]decane was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (Silica gel, petroleum ether/ethyl acetate=1/1) to give desired 8-(4-chloro-1H-indazol-3-yl)-7-(trifluoromethyl)-1, 4-dioxa-8-azaspiro [4.5]decane (140 mg, crude) as a yellow solid. MS (ESI): mass calcd. For C₁₅H₁₅ClF₃N₃O₂ 361.08, mass found 362.0 [M+H]⁺.

Step 6: To a solution of 4-isopropylsulfonylbenzenesulfonyl chloride (164 mg, 581 mol, 1.5 eq) and 8-(4-chloro-1H-indazol-3-yl)-7-(trifluoromethyl)-1, 4-dioxa-8-azaspiro [4.5]decane (140 mg, 387 μmol, 1 eq) in DCM (1 mL) was added TEA (78.3 mg, 774 μmol, 108 μL, 2 eq) and DMAP (4.73 mg, 38.7 μmol, 0.1 eq) at 0° C. The mixture was stirred at 20° C. for 0.5 hour. LC-MS showed 8-(4-chloro-1H-indazol-3-yl)-7-(trifluoromethyl)-1, 4-dioxa-8-azaspiro [4.5]decane was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (Silica gel, petroleum ether/ethyl acetate=2/1) to give desired 8-[4-chloro-1-(4-isopropylsulfonylphenyl) sulfonyl-indazol-3-yl]-7-(trifluoromethyl)-1, 4-dioxa-8-azaspiro [4.5]decane (70 mg, crude) as a yellow oil. MS (ESI): mass calcd. For C₂₄H₂₅ClF₃N₃O₆S₂ 607.08, mass found 608.0 [M+H]⁺.

Step 7: To a solution of 8-[4-chloro-1-(4-isopropylsulfonylphenyl) sulfonyl-indazol-3-yl]-7-(trifluoromethyl)-1, 4-dioxa-8-azaspiro [4.5]decane (25 mg, 41.1 μmol, 1 eq) in dioxane (1 mL) and H₂O (0.6 mL) was added HCl (12 M, 42.8 μL, 12.5 eq). The mixture was stirred at 85° C. for 12 hours. LC-MS showed 8-[4-chloro-1-(4-isopropylsulfonylphenyl) sulfonyl-indazol-3-yl]-7-(trifluoromethyl)-1, 4-dioxa-8-azaspiro [4.5]decane was consumed completely and desired mass was detected. The pH value of the reaction mixture was adjusted to pH˜7 with NaOH (2M), extracted with DCM (5 mL*3). The combined organic layers were washed with brine (5 mL) and dried over Na₂SO₄, filtered and concentrated under reduced pressure to give a residue. The residue was purified by prep-HPLC (neutral condition; column: Waters Xbridge BEH C18 100*30 mm*10 μm; mobile phase: [water(NH₄HCO₃)-ACN]; B %: 25%-55%, 8 min) to give desired 1-[4-chloro-1-(4-isopropylsulfonylphenyl)sulfonyl-indazol-3-yl]-2-(trifluoromethyl)piperidin-4-one (8.6 mg, 15.24 μmol, 37.08% yield, 99.97% purity) as a white solid. ¹H NMR (400 MHz, DMSO-d₆) δ 8.19-8.08 (m, 3H), 8.07-7.96 (m, 2H), 7.72 (t, J=8.1 Hz, 1H), 7.56 (d, J=7.6 Hz, 1H), 5.12-5.00 (m, 1H), 3.84-3.67 (m, 2H), 3.50-3.46 (m, 1H), 3.27 (dd, J=7.5, 16.0 Hz, 1H), 2.61 (br d, J=2.9 Hz, 1H), 2.57 (br dd, J=3.0, 6.5 Hz, 1H), 2.46-2.38 (m, 1H), 1.09 (dd, J=2.9, 6.8 Hz, 6H) HPLC: 99.97% (220 nm), 99.80% (210 nm), 100.00% (254 nm). MS (ESI): mass calcd. For C₂₂H₂₁ClF₃N₃O₅S₂ 563.06, mass found 564.0 [M+H]⁺.

### Compound 85: 4-chloro-3-[4, 4-difluoro-2-(trifluoromethyl)-1-piperidyl]-1-(4-isopropylsulfonylphenyl) sulfonyl-indazole

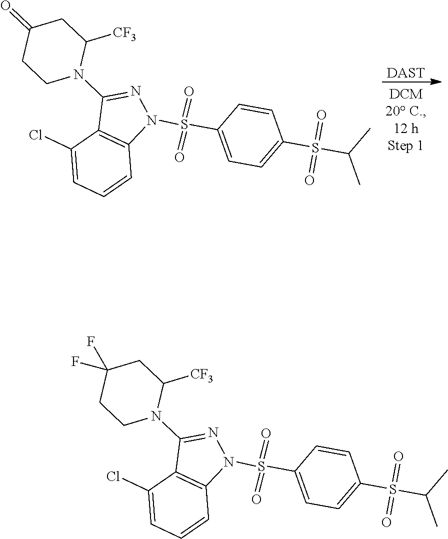 [SMILES: CC(C)S(=O)(=O)c1ccc(S(=O)(=O)n2nc(N3CCC(=O)CC3C(F)(F)F)c3c(Cl)cccc32)cc1 ; "DAST" ; CC(C)S(=O)(=O)c1ccc(S(=O)(=O)n2nc(N3CCC(F)(F)CC3C(F)(F)F)c3c(Cl)cccc32)cc1]

Step 1: To a solution of 1-[4-chloro-1-(4-isopropylsulfonylphenyl) sulfonyl-indazol-3-yl]-2-(trifluoromethyl) piperidin-4-one (40 mg, 70.9 μmol, 1 eq) in DCM (1 mL) was added DAST (22.9 mg, 142 μmol, 18.7 μL, 2 eq). The mixture was stirred at 20° C. for 12 hours. LC-MS showed 1-[4-chloro-1-(4-isopropylsulfonylphenyl) sulfonyl-indazol-3-yl]-2-(trifluoromethyl) piperidin-4-one was consumed completely and desired mass was detected. The reaction mixture was added MeOH (1 mL) and then was concentrated to give the crude product. The residue was purified by prep-HPLC (TFA condition; column: Phenomenex Luna C18 75*30 mm*3 μm; mobile phase: [water(TFA)-ACN]; B %: 45%-75%, 8 min) to give desired 4-chloro-3-[4, 4-difluoro-2-(trifluoromethyl)-1-piperidyl]-1-(4-isopropylsulfonylphenyl) sulfonyl-indazole (2 mg, 3.37 μmol, 4.75% yield, 98.72% purity) as a white solid. ¹H NMR (400 MHz, DMSO-d₆) δ 8.13 (d, J=8.6 Hz, 3H), 8.06-8.00 (m, 2H), 7.72 (t, J=8.1 Hz, 1H), 7.55 (d, J=7.8 Hz, 1H), 4.79 (br t, J=7.6 Hz, 1H), 3.68-3.57 (m, 2H), 3.48 (br d, J=6.9 Hz, 1H), 2.69-2.55 (m, 2H), 2.17-2.03 (m, 1H), 1.98-1.78 (m, 1H), 1.09 (dd, J=2.1, 6.8 Hz, 6H). HPLC: 98.72% (220 nm), 98.61% (210 nm), 99.03% (254 nm). MS (ESI): mass calcd. For C₂₂H₂₁ClF₅N₃O₄S₂ 585.06, mass found 586.0 [M+H]⁺.

### Compound 86: 4-chloro-3-[4-(difluoromethoxy)-2-(trifluoromethyl)-1-piperidyl]-1-(4-isopropylsulfonylphenyl) sulfonyl-indazole

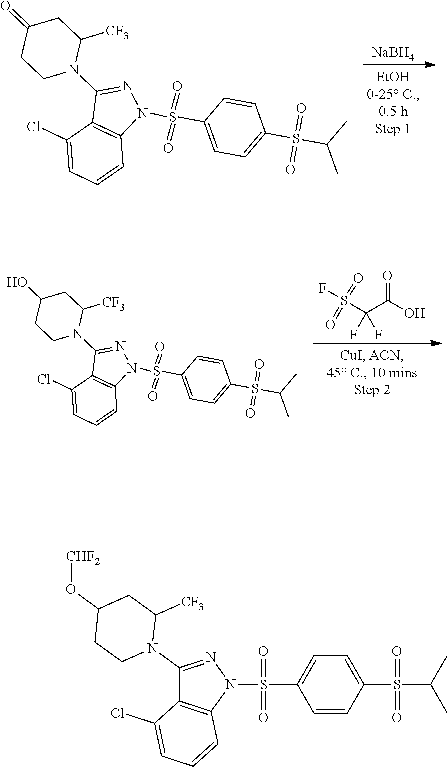 [SMILES: CC(C)S(=O)(=O)c1ccc(S(=O)(=O)n2nc(N3CCC(=O)CC3C(F)(F)F)c3c(Cl)cccc32)cc1 ; "NaBH4" ; CC(C)S(=O)(=O)c1ccc(S(=O)(=O)n2nc(N3CCC(O)CC3C(F)(F)F)c3c(Cl)cccc32)cc1 ; O=C(O)C(F)(F)S(=O)(=O)F ; CC(C)S(=O)(=O)c1ccc(S(=O)(=O)n2nc(N3CCC(OC(F)F)CC3C(F)(F)F)c3c(Cl)cccc32)cc1]

Step 1: To a solution of 1-[4-chloro-1-(4-isopropylsulfonylphenyl) sulfonyl-indazol-3-yl]-2-(trifluoromethyl) piperidin-4-one (0.2 g, 355 μmol, 1 eq) in EtOH (1 mL) was added NaBH₄ (20.1 mg, 532 μmol, 1.5 eq) at 0° C. The mixture was stirred at 25° C. for 0.5 hour. LC-MS showed 1-[4-chloro-1-(4-isopropylsulfonylphenyl) sulfonyl-indazol-3-yl]-2-(trifluoromethyl) piperidin-4-one was consumed completely and one main peak with desired mass was detected. The residue was diluted with HCl (1N, 8 mL) and the resulting mixture was allowed to return to room temperature. The EtOH was evaporated in vacuo. The reaction mixture was added to water (20 mL), extracted with DCM (10 mL*3). The combined organic layers were washed with brine (20 mL) and dried over Na₂SO₄, filtered and concentrated under reduced pressure to give desired 1-[4-chloro-1-(4-isopropylsulfonylphenyl)sulfonyl-indazol-3-yl]-2-(trifluoromethyl)piperidin-4-ol (0.2 g, crude) as a yellow oil. MS (ESI): mass calcd. For C₂₂H₂₃S₂O₅N₃ClF₃ 565.07, mass found 566.0 [M+H]⁺.

Step 2: To a solution of 1-[4-chloro-1-(4-isopropylsulfonylphenyl) sulfonyl-indazol-3-yl]-2-(trifluoromethyl) piperidin-4-ol (150 mg, 265 μmol, 1 eq) in ACN (2 mL) was added CuI (10.1 mg, 53.0 μmol, 0.2 eq) and heated to 45° C. under N₂ atmosphere. To this mixture was added a solution of 2, 2-difluoro-2-fluorosulfonyl-acetic acid (61.4 mg, 345 μmol, 1.3 eq) in ACN (0.5 mL) was added dropwise at 45° C. The resulting mixture was stirred at 45° C. for 10 mins. LC-MS showed 1-[4-chloro-1-(4-isopropylsulfonylphenyl) sulfonyl-indazol-3-yl]-2-(trifluoromethyl) piperidin-4-ol was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-HPLC (TFA condition; Method: column: Phenomenex Luna C18 75*30 mm*3 μm; mobile phase: [water(TFA)-ACN]; B %: 50%-75%, 8 min) to give desired 4-chloro-3-[4-(difluoromethoxy)-2-(trifluoromethyl)-1-piperidyl]-1-(4-isopropylsulfonylphenyl)sulfonyl-indazole (8.4 mg, 13.6 μmol, 5.15% yield, 100.0% purity) as a yellow solid. ¹H NMR (400 MHz, DMSO-d₆) δ 8.15-8.06 (m, 3H), 8.06-7.99 (m, 2H), 7.73-7.65 (m, 1H), 7.55-7.49 (m, 1H), 6.98-6.57 (m, 1H), 4.59-4.47 (m, 2H), 3.68-3.58 (m, 1H), 3.51-3.42 (m, 1H), 3.28-3.20 (m, 1H), 2.41-2.31 (m, 1H), 2.17-2.04 (m, 1H), 1.82-1.67 (m, 2H), 1.08 (d, J=6.8 Hz, 6H). HPLC: 100.00% (220 nm), 100.00% (215 nm), 100.00% (254 nm). MS (ESI): mass calcd. For C₂₃H₂₃S₂O₅N₃ClF₉ 615.07, mass found 616.0[M+H]⁺.

### Compound 87: 4-chloro-3-[7-(difluoromethoxy)-4-azaspiro [2.5]octan-4-yl]-1-(4-isopropylsulfonylphenyl) sulfonyl-indazole

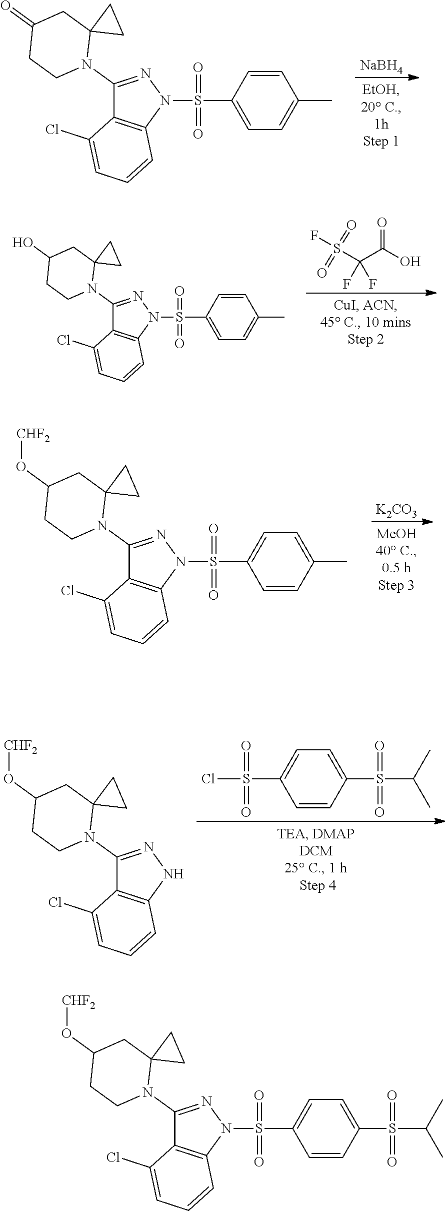

Step 1: To a solution of 4-[4-chloro-1-(p-tolylsulfonyl) indazol-3-yl]-4-azaspiro [2.5]octan-7-one (0.5 g, 1.16 mmol, 1 eq) in EtOH (5 mL) was added NaBH₄ (66.0 mg, 1.74 mmol, 1.5 eq). The mixture was stirred at 20° C. for 1 hour. LC-MS showed 4-[4-chloro-1-(p-tolylsulfonyl) indazol-3-yl]-4-azaspiro [2.5]octan-7-one was consumed completely and one main peak with desired mass was detected. The residue was diluted with HCl (1 N, 8 mL) and the resulting mixture was allowed to return to room temperature. The EtOH was evaporated in vacuo. The reaction mixture was added to water (20 mL), extracted with DCM (10 mL*3). The combined organic layers were washed with brine (20 mL) and dried over Na₂SO₄, filtered and concentrated under reduced pressure to give a residue. The residue was purified by prep-TLC (Silica gel, petroleum ether/EtOAc=2/1) to give desired 4-[4-chloro-1-(p-tolylsulfonyl) indazol-3-yl]-4-azaspiro [2.5]octan-7-ol (0.4 g, 926 μmol, 79.6% yield) as a yellow solid. MS (ESI): mass calcd. For C₂₁H₂₂N₃ClO₃S 431.11, mass found 432.1[M+H]⁺.

Step 2: To a solution of 4-[4-chloro-1-(p-tolylsulfonyl) indazol-3-yl]-4-azaspiro [2.5]octan-7-ol (0.2 g, 463 μmol, 1 eq) in ACN (2 mL) was added CuI (17.6 mg, 92.6 μmol, 0.2 eq) and heated to 45° C. under N₂ atmosphere. To this mixture was added a solution of 2, 2-difluoro-2-fluorosulfonyl-acetic acid (107 mg, 602 μmol, 1.3 eq) in ACN (0.5 mL) was added dropwise at 45° C. The resulting mixture was stirred at 45° C. for 0.5 hour. LC-MS showed 4-[4-chloro-1-(p-tolylsulfonyl) indazol-3-yl]-4-azaspiro [2.5]octan-7-ol was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-TLC (Silica gel, petroleum ether/EtOAc=4/1) to give desired 4-chloro-3-[7-(difluoromethoxy)-4-azaspiro [2.5]octan-4-yl]-1-(p-tolylsulfonyl) indazole (150 mg, 311 μmol, 67.2% yield) as a yellow oil. MS (ESI): mass calcd. For C₂₂H₂₂N₃ClO₃SF₂ 481.1, mass found 482.0[M+H]⁺.

Step 3: To a solution of 4-chloro-3-[7-(difluoromethoxy)-4-azaspiro [2.5]octan-4-yl]-1-(p-tolylsulfonyl) indazole (150 mg, 311 μmol, 1 eq) in MeOH (2 mL) was added K₂CO₃ (108 mg, 778 μmol, 2.5 eq). The mixture was stirred at 40° C. for 0.5 hour. LC-MS showed 4-chloro-3-[7-(difluoromethoxy)-4-azaspiro [2.5]octan-4-yl]-1-(p-tolylsulfonyl) indazole was consumed completely and one main peak with desired mass was detected. The residue was diluted with H₂O (10 mL) and extracted with Ethyl acetate (5 mL*3). The combined organic layers were washed with brine (10 mL), dried over Na₂SO₄, filtered and concentrated under reduced pressure to give a residue. The residue was purified by prep-TLC (Silica gel, petroleum ether/EtOAc=3/1) to give desired 4-chloro-3-[7-(difluoromethoxy)-4-azaspiro [2.5]octan-4-yl]-1H-indazole (40 mg, 122 μmol, 39.1% yield) as a yellow solid. MS (ESI): mass calcd. For C₁₅H₁₆N₃ClOF₂ 327.1, mass found 328.0[M+H]⁺.

Step 4: To a solution of 4-chloro-3-[7-(difluoromethoxy)-4-azaspiro[2.5]octan-4-yl]-1H-indazole (40 mg, 122 μmol, 1 eq) and 4-isopropylsulfonylbenzenesulfonyl chloride (86.3 mg, 305 μmol, 2.5 eq) in DCM (2 mL) was added TEA (37.1 mg, 366 μmol, 3 eq) and DMAP (1.49 mg, 12.2 μmol, 0.1 eq). The mixture was stirred at 25° C. for 1 hour. LC-MS showed 4-chloro-3-[7-(difluoromethoxy)-4-azaspiro [2.5]octan-4-yl]-1H-indazole 1 was consumed completely and one main peak desired mass was detected. The residue was diluted with H₂O (10 mL) and extracted with Ethyl acetate (5 mL*3). The combined organic layers were washed with brine (10 mL), dried over Na₂SO₄, filtered and concentrated under reduced pressure to give a residue. The residue was purified by prep-HPLC (TFA condition; Method: column: Phenomenex Luna C18 75*30 mm*3 μm; mobile phase: [water(TFA)-ACN]; B %: 55%-85%, 8 min) to give desired 4-chloro-3-[7-(difluoromethoxy)-4-azaspiro[2.5]octan-4-yl]-1-(4-isopropylsulfonylphenyl)sulfonyl-indazole (41.2 mg, 71.8 μmol, 58.8% yield, 100.00% purity) as a yellow solid. ¹H NMR (400 MHz, DMSO-d₆) δ 8.10-7.99 (m, 5H), 7.64 (t, J=8.1 Hz, 1H), 7.46 (d, J=7.5 Hz, 1H), 6.96-6.54 (m, 1H), 4.40 (br d, J=3.5 Hz, 1H), 3.65-3.50 (m, 2H), 3.42-3.25 (m, 2H), 1.75 (br d, J=4.0 Hz, 2H), 1.38-1.21 (m, 1H), 1.09 (dd, J=1.0, 6.8 Hz, 6H), 0.73-0.54 (m, 2H), 0.42-0.13 (m, 2H). HPLC: 100.00% (220 nm), 100.00% (215 nm), 100.00% (254 nm). MS (ESI): mass calcd. For C₂₄H₂₆S₂O₅N₃ClF₂ 573.1, mass found 574.0 [M+H]⁺.

### Compound 88: 11-[4-chloro-1-[4-(1, 1-difluoroethyl) phenyl]sulfonyl-indazol-3-yl]-8-oxa-11-azadispiro [2.1.35.33]undecane

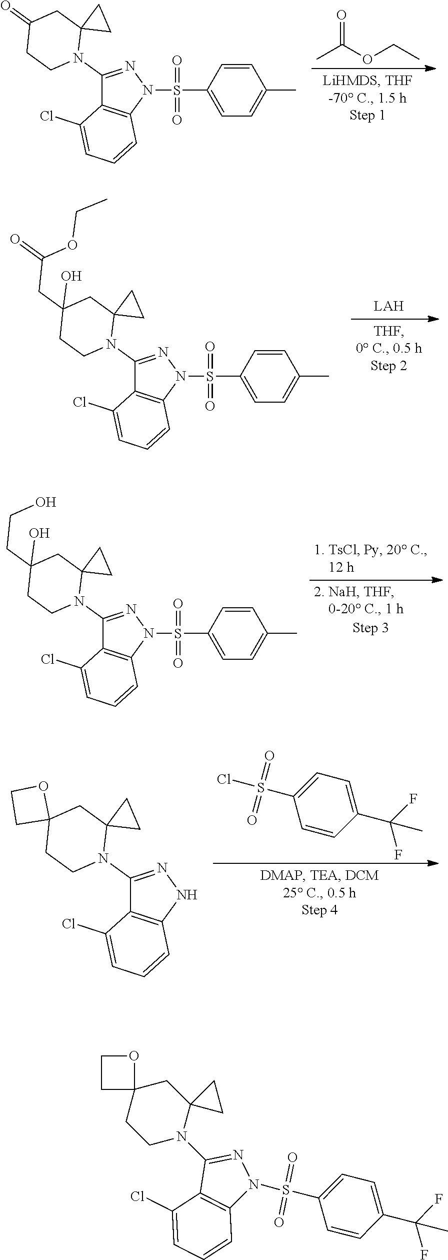

Step 1: To a solution of EtOAc (215 mg, 2.44 mmol, 239 μL, 1.5 eq) in THF (1 mL) was added dropwise LiHMDS (1 M, 2.44 mL, 1.5 eq) at −70° C. After addition, the mixture was stirred at this temperature for 0.5 hour, and then 4-[4-chloro-1-(p-tolylsulfonyl) indazol-3-yl]-4-azaspiro[2.5]octan-7-one (0.7 g, 1.63 mmol, 1 eq) in THF (7 mL) was added dropwise at −70° C. The resulting mixture was stirred at −70° C. for 1 hour. LC-MS showed 4-[4-chloro-1-(p-tolylsulfonyl) indazol-3-yl]-4-azaspiro [2.5]octan-7-one was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜25% EtOAc/petroleum ether gradient @100 mL/min) to give desired ethyl 2-[4-[4-chloro-1-(p-tolylsulfonyl) indazol-3-yl]-7-hydroxy-4-azaspiro [2.5]octan-7-yl]acetate (0.63 g, 1.22 mmol, 74.7% yield) as a yellow solid. MS (ESI): mass calcd. For C₂₅H₂₈N₃SO₅Cl 517.14, mass found 518.0[M+H]⁺.

Step 2: To a solution of ethyl 2-[4-[4-chloro-1-(p-tolylsulfonyl) indazol-3-yl]-7-hydroxy-4-azaspiro [2.5]octan-7-yl]acetate (0.4 g, 772 μmol, 1 eq) in THF (4 mL) was added LAH (87.9 mg, 2.32 mmol, 3 eq) at 0° C. The mixture was stirred at 0° C. for 0.5 hour. LC-MS showed ethyl 2-[4-[4-chloro-1-(p-tolylsulfonyl) indazol-3-yl]-7-hydroxy-4-azaspiro [2.5]octan-7-yl]acetate was consumed completely and one main peak with desired mass was detected. The reaction mixture was quenched by addition of 0.09 mL of H₂O at 0° C., followed by 0.09 mL of 15% aqueous NaOH at 0° C., followed by 0.27 mL of H₂O at 0° C. under N₂. After being stirred at room temperature for 0.5 hour, the solid was removed by filtration. The filtrate was concentrated to dryness to give desired 4-[4-chloro-1-(p-tolylsulfonyl) indazol-3-yl]-7-(2-hydroxyethyl)-4-azaspiro [2.5]octan-7-ol (0.37 g, crude) as a yellow oil. MS (ESI): mass calcd. For C₂₃H₂₆N₃SO₄Cl 475.13, mass found 476.1[M+H]⁺.

Step 3: To a solution of 4-[4-chloro-1-(p-tolylsulfonyl) indazol-3-yl]-7-(2-hydroxyethyl)-4-azaspiro [2.5]octan-7-ol (370 mg, 777 μmol, 1 eq) in Py (4 mL) was added 4-methylbenzenesulfonyl chloride (193 mg, 1.01 mmol, 1.3 eq). The mixture was stirred at 20° C. for 12 hour. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue in THF (4 mL) was added NaH (124 mg, 3.11 mmol, 60% purity, 4 eq) at 0° C. The mixture was stirred at 20° C. for 1 hour. LC-MS showed 4-[4-chloro-1-(p-tolylsulfonyl) indazol-3-yl]-7-(2-hydroxyethyl)-4-azaspiro [2.5]octan-7-ol was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-TLC (Silica gel, petroleum ether/EtOAc=1/1) to give desired 11-(4-chloro-1H-indazol-3-yl)-8-oxa-11-azadispiro [2.1.35.33]undecane (15 mg, 49.4 μmol, 6.35% yield) as a yellow solid. MS (ESI): mass calcd. For C₁₆H₁₈N₃ClO 303.11, mass found 304.1[M+H]⁺.

Step 4: To a solution of 11-(4-chloro-1H-indazol-3-yl)-8-oxa-11-azadispiro [2.1.35.33]undecane (12 mg, 39.5 μmol, 1 eq) and 4-(1,1-difluoroethyl)benzenesulfonyl chloride (19.0 mg, 79.0 μmol, 2 eq) in DCM (1 mL) was added TEA (9.99 mg, 98.8 μmol, 2.5 eq) and DMAP (483 pg, 3.95 μmol, 0.1 eq). The mixture was stirred at 25° C. for 0.5 hour. LC-MS showed 11-(4-chloro-1H-indazol-3-yl)-8-oxa-11-azadispiro [2.1.35.33]undecane was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-HPLC (neutral condition; Method: column: Waters Xbridge BEH C18 100*30 mm*10 μm; mobile phase: [water(NH₄HCO₃)-ACN]; B %: 50%-95%, 8 min) to give desired 11-[4-chloro-1-[4-(1,1-difluoroethyl)phenyl]sulfonyl-indazol-3-yl]-8-oxa-11-azadispiro[2.1.35.33]undecane (5.4 mg, 10.6 μmol, 26.9% yield, 100% purity) as a yellow solid. ¹H NMR (400 MHz, DMSO-d₆) δ 8.08-8.04 (m, 1H), 7.88-7.83 (m, 2H), 7.75-7.70 (m, 2H), 7.65-7.59 (m, 1H), 7.45-7.41 (m, 1H), 4.45-4.35 (m, 2H), 3.49 (br d, J=14.5 Hz, 4H), 3.31-3.17 (m, 2H), 2.47-2.30 (m, 2H), 1.91 (t, J=19.1 Hz, 3H), 0.71-0.59 (m, 2H), 0.24 (br s, 2H). HPLC: 100% (220 nm), 100% (215 nm), 100% (254 nm). MS (ESI): mass calcd. For C₂₄H₂₄N₃SO₃ClF₂507.12, mass found 508.1[M+H]⁺.

### Compound 89: 4-chloro-1-[4-(3-fluorooxetan-3-yl) phenyl]sulfonyl-3-[7-(5-fluoropyrimidin-2-yl)-4, 7-diazaspiro [2.5]octan-4-yl]indazole

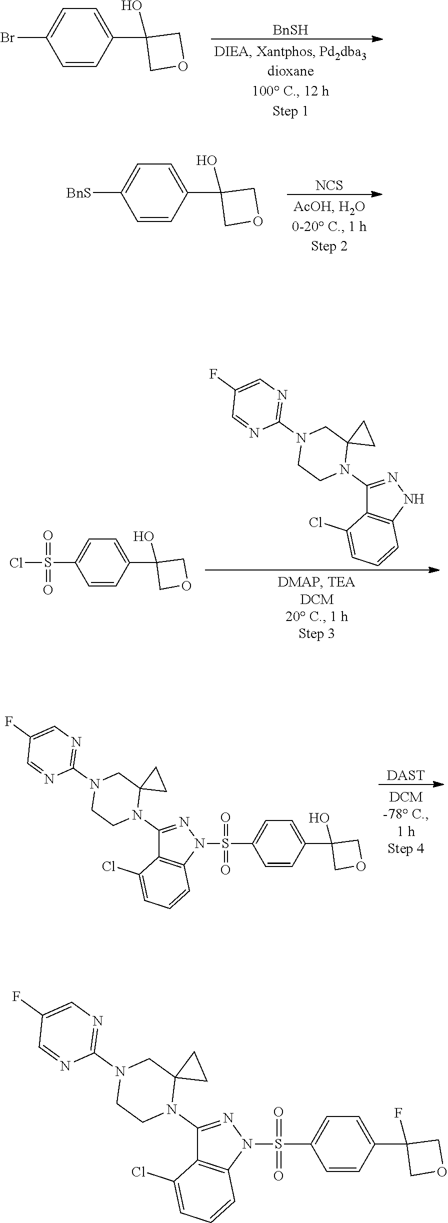

Step 1: A mixture of 3-(4-bromophenyl)oxetan-3-ol (500 mg, 2.18 mmol, 1 eq), BnSH (325 mg, 2.62 mmol, 307 μL, 1.2 eq), Xantphos (63.2 mg, 109 μmol, 0.05 eq), Pd₂(dba)₃ (200 mg, 218 μmol, 0.1 eq) and DIEA (564 mg, 4.37 mmol, 761 μL, 2 eq) in dioxane (5 mL) was degassed and purged with N₂ for 3 times, and then the mixture was stirred at 100° C. for 12 hours under N₂ atmosphere. TLC indicated 3-(4-bromophenyl) oxetan-3-ol was consumed completely and one major new spot with larger polarity was detected. The reaction mixture was partitioned between water (10 mL) and EtOAc (10 mL). The organic phase was separated, washed with brine (10 mL), dried over Na₂SO₄, filtered and concentrated under reduced pressure to give a residue. The residue was purified by prep-TLC (Silica gel, petroleum ether/ethyl acetate=1/1) to give desired 3-(4-benzylsulfanylphenyl) oxetan-3-ol (540 mg, 1.98 mmol, 90.8% yield) as a yellow solid.

Step 2: To a solution of 3-(4-benzylsulfanylphenyl) oxetan-3-ol (440 mg, 1.62 mmol, 1 eq) in AcOH (4 mL) and H₂O (1 mL) was added NCS (647 mg, 4.85 mmol, 3 eq) at 0° C. The mixture was stirred at 20° C. for 1 hour. TLC indicated 3-(4-benzylsulfanylphenyl) oxetan-3-ol was consumed, and one major new spot with larger polarity was detected. Then it was partitioned between 5 mL of water and 15 mL of ethyl acetate. The organic phase was separated, washed with 5 mL of brine, dried over Na₂SO₄, filtered and concentrated under reduced pressure to give a residue. The residue was purified by prep-TLC (Silica gel, petroleum ether/ethyl acetate=1/1) to give desired 4-(3-hydroxyoxetan-3-yl) benzenesulfonyl chloride (150 mg, 603 μmol, 37.3% yield) as a white gum.

Step 3: To a solution of 4-chloro-3-[7-(5-fluoropyrimidin-2-yl)-4,7-diazaspiro[2.5]octan-4-yl]-1H-indazole (100 mg, 279 μmol, 1 eq), 4-(3-hydroxyoxetan-3-yl)benzenesulfonyl chloride (139 mg, 557 μmol, 2 eq) and TEA (84.6 mg, 836 μmol, 116 μL, 3 eq) in DCM (3 mL) was added DMAP (34.1 mg, 279 μmol, 1 eq). The mixture was stirred at 20° C. for 0.5 hour. LC-MS showed 4-chloro-3-[7-(5-fluoropyrimidin-2-yl)-4, 7-diazaspiro [2.5]octan-4-yl]-1H-indazole remained. Several new peaks were shown on LC-MS and desired compound was detected. The reaction mixture was concentrated under reduced pressure to give a residue. The residue was purified by prep-TLC (Silica gel, petroleum ether/ethyl acetate=1/1) to give desired 3-[4-[4-chloro-3-[7-(5-fluoropyrimidin-2-yl)-4, 7-diazaspiro [2.5]octan-4-yl]indazol-1-yl]sulfonylphenyl]oxetan-3-ol (100 mg, 175 μmol, 62.8% yield) as a colorless oil. MS (ESI): mass calcd. For C₂₆H₂₄SO₄N₆ClF 570.13 mass found 571.1[M+H]⁺.

Step 4: To a solution of 3-[4-[4-chloro-3-[7-(5-fluoropyrimidin-2-yl)-4,7-diazaspiro[2.5]octan-4-yl]indazol-1-yl]sulfonylphenyl]oxetan-3-ol (90 mg, 158 μmol, 1 eq) in DCM (2 mL) was added dropwise DAST (30.5 mg, 189 μmol, 25.0 μL, 1.2 eq) at −78° C. under N₂. The mixture was stirred at −78° C. for 0.5 hour. LC-MS showed 3-[4-[4-chloro-3-[7-(5-fluoropyrimidin-2-yl)-4, 7-diazaspiro [2.5]octan-4-yl]indazol-1-yl]sulfonylphenyl]oxetan-3-ol was consumed completely and one main peak with desired mass was detected. Then it was partitioned between 5 mL of water and 15 mL of ethyl acetate. The organic phase was separated, washed with 5 mL of brine, dried over Na₂SO₄, filtered and concentrated under reduced pressure to give a residue. The residue was purified by prep-HPLC (column: Phenomenex Luna C18 75*30 mm*3 μm; mobile phase: [water(TFA)-ACN]; B %: 55%-85%, 8 min) to give desired 4-chloro-1-[4-(3-fluorooxetan-3-yl)phenyl]sulfonyl-3-[7-(5-fluoropyrimidin-2-yl)-4,7-diazaspiro[2.5]octan-4-yl]indazole (23.9 mg, 40.2 μmol, 25.5% yield, 96.55% purity) as a white solid. ¹H NMR (400 MHz, DMSO-d₆) δ 8.47 (s, 2H), 8.08 (d, J=8.5 Hz, 1H), 7.87 (d, J=8.4 Hz, 2H), 7.69 (d, J=8.5 Hz, 2H), 7.64 (t, J=8.1 Hz, 1H), 7.46 (d, J=7.8 Hz, 1H), 4.94 (d, J=9.0 Hz, 1H), 4.88 (d, J=9.0 Hz, 1H), 4.80 (d, J=9.1 Hz, 1H), 4.74 (d, J=9.0 Hz, 1H), 3.92 (s, 2H), 3.61-3.57 (m, 4H), 0.79 (s, 2H), 0.42 (br s, 2H). HPLC: 96.55% (220 nm), 95.87% (215 nm), 96.40% (254 nm). MS (ESI): mass calcd. For C₂₆H₂₃SO₃N₆ClF₂ 572.12 mass found 573.0[M+H]⁺.

### Compound 90: 4-chloro-1-[4-(1, 1-difluoro-2-methyl-propyl) phenyl]sulfonyl-3-[7-(5-fluoropyrimidin-2-yl)-4, 7-diazaspiro [2.5]octan-4-yl]indazole

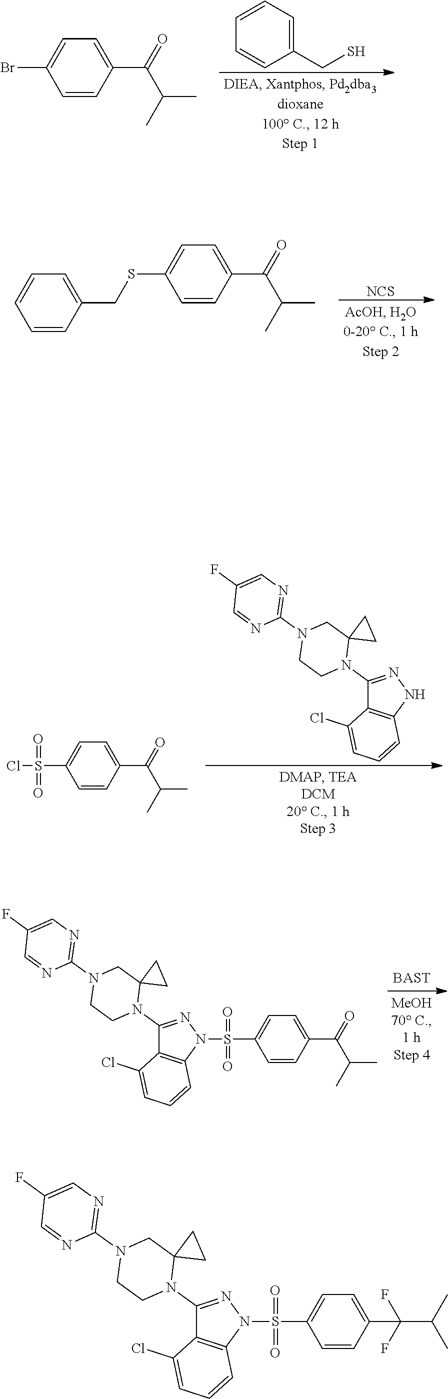

Step 1: A mixture of 1-(4-bromophenyl)-2-methyl-propan-1-one (2 g, 8.81 mmol, 1 eq), phenylmethanethiol (1.20 g, 9.69 mmol, 1.14 mL, 1.1 eq), Pd₂(dba)₃ (403 mg, 440 μmol, 0.05 eq), Xantphos (510 mg, 88 μmol, 0.1 eq) and DIEA (2.28 g, 17.6 mmol, 3.07 mL, 2 eq) in dioxane (20 mL) was degassed and purged with N₂ for 3 times, and then the mixture was stirred at 100° C. for 12 hours under N₂ atmosphere. TLC indicated 1-(4-bromophenyl)-2-methyl-propan-1-one (2 g, 8.81 mmol, 1 eq), phenylmethanethiol was consumed completely, and one major new spot with larger polarity was detected. The reaction mixture was partitioned between water (50 mL) and EtOAc (50 mL). The organic phase was separated, washed with brine (50 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜10% Ethyl acetate/petroleum ether gradient @60 mL/min) to give desired 1-(4-benzylsulfanylphenyl)-2-methyl-propan-1-one (1.9 g, 7.03 mmol, 79.8% yield) as a white solid.

Step 2: To a solution of 1-(4-benzylsulfanylphenyl)-2-methyl-propan-1-one (1 g, 3.70 mmol, 1 eq) in CH₃COOH (6 mL) and H₂O (2 mL) was added NCS (1.48 g, 11.1 mmol, 3 eq) at 0° C. The mixture was stirred at 20° C. for 1 hour. TLC indicated 1-(4-benzylsulfanylphenyl)-2-methyl-propan-1-one (1 g, 3.70 mmol, 1 eq) was remained, and one major new spot with larger polarity was detected. The reaction mixture was diluted with water (10 mL) and extracted with MTBE (5 mL*6). The combined organic layers were washed with brine 10 mL, dried over NaSO₄, filtered and concentrated under reduced pressure to give a residue. The residue was purified by flash silica gel chromatography (ISCO®; 20 g SepaFlash® Silica Flash Column, Eluent of 0˜8% Ethyl acetate/petroleum ether gradient @80 mL/min) to give desired 4-(2-methylpropanoyl) benzenesulfonyl chloride (700 mg, 2.84 mmol, 87.5% yield) as a yellow oil.

Step 3: To a solution of 4-chloro-3-[7-(5-fluoropyrimidin-2-yl)-4, 7-diazaspiro [2.5]octan-4-yl]-1H-indazole (100 mg, 279 μmol, 1 eq) in DCM (2 mL) was added DMAP (17.0 mg, 139 μmol, 0.5 eq), 4-(2-methylpropanoyl)benzenesulfonyl chloride (138 mg, 557 μmol, 2 eq) and TEA (84.6 mg, 836 μmol, 116 μL, 3 eq). The mixture was stirred at 20° C. for 1 hour. TLC indicated 4-chloro-3-[7-(5-fluoropyrimidin-2-yl)-4, 7-diazaspiro [2.5]octan-4-yl]-1H-indazole was consumed completely and one new spot formed. The reaction was clean according to TLC. The reaction concentrated under reduced pressure to give a residue. The residue was purified by prep-TLC (Silica gel, petroleum ether/ethyl acetate=1/1) to give desired 1-[4-[4-chloro-3-[7-(5-fluoropyrimidin-2-yl)-4, 7-diazaspiro [2.5]octan-4-yl]indazol-1-yl]sulfonylphenyl]-2-methyl-propan-1-one (100 mg, 176 μmol, 63.0% yield) as a yellow oil. MS (ESI): mass calcd. For C₂₇H₂₆SO₃N₆ClF 568.15 mass found 569.1 [M+H]⁺.

Step 4: To a solution of 1-[4-[4-chloro-3-[7-(5-fluoropyrimidin-2-yl)-4, 7-diazaspiro [2.5]octan-4-yl]indazol-1-yl]sulfonylphenyl]-2-methyl-propan-1-one (60 mg, 105 μmol, 1 eq) in DAST (1 mL) was stirred at 70° C. for 1 hour. LC-MS showed 1-[4-[4-chloro-3-[7-(5-fluoropyrimidin-2-yl)-4, 7-diazaspiro [2.5]octan-4-yl]indazol-1-yl]sulfonylphenyl]-2-methyl-propan-1-one remained. Several new peaks were shown on LC-MS and desired compound was detected. The reaction mixture was quenched by sat.aq.NaHCO₃ (1 ml), then extracted with EtOAc (4 ml*2), the organic phase was separated, washed with brine (2 ml), dried over Na₂SO₄, filtered and concentrated under reduced pressure to give the crude product. The residue was purified by prep-HPLC (TFA condition; column: Phenomenex Luna C18 75*30 mm*3 μm; mobile phase: [water(TFA)-ACN]; B %: 60%-90%, 8 min) to give desired 4-chloro-1-[4-(1,1-difluoro-2-methyl-propyl)phenyl]sulfonyl-3-[7-(5-fluoropyrimidin-2-yl)-4,7-diazaspiro[2.5]octan-4-yl]indazole (15.5 mg, 25.3 μmol, 24.0% yield, 96.5% purity) as a gray solid. ¹H NMR (400 MHz, DMSO-d₆) δ 8.45 (s, 2H), 8.08 (d, J=8.5 Hz, 1H), 7.89 (br d, J=7.9 Hz, 2H), 7.63 (br d, J=8.1 Hz, 3H), 7.46 (d, J=7.5 Hz, 1H), 3.90 (s, 2H), 3.63-3.57 (m, 4H), 2.42-2.27 (m, 1H), 0.81 (br d, J=6.6 Hz, 6H), 0.74 (br s, 2H), 0.34 (br s, 2H). MS (ESI): mass calcd. For C₂₇H₂₆F₃SO₂N₆Cl 590.15 mass found 591.3 [M+H]⁺.

### Compound 91: 4-chloro-1-[4-(1, 1-difluoroethyl) phenyl]sulfonyl-6-fluoro-3-[7-(5-fluoropyrimidin-2-yl)-4, 7-diazaspiro [2.5]octan-4-yl]indazole

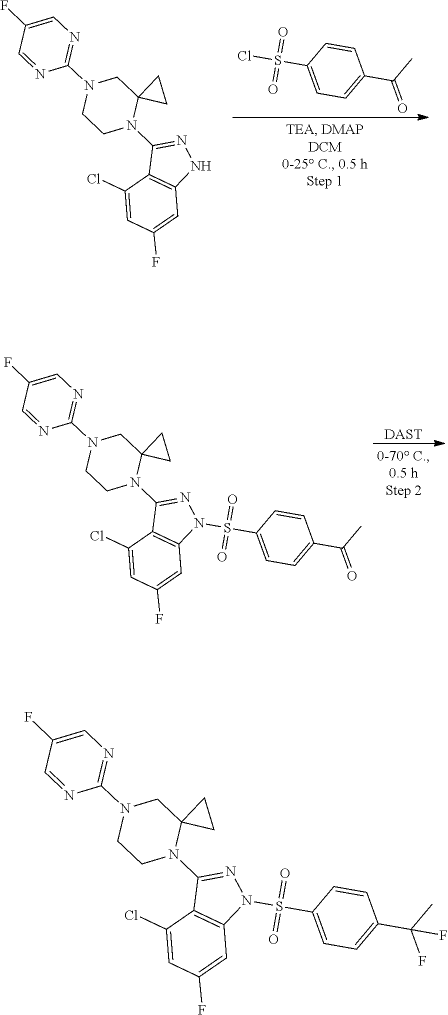 [SMILES: Fc1cnc(N2CCN(c3n[nH]c4cc(F)cc(Cl)c34)C3(CC3)C2)nc1 ; CC(=O)c1ccc(S(=O)(=O)Cl)cc1 ; CC(=O)c1ccc(S(=O)(=O)n2nc(N3CCN(c4ncc(F)cn4)CC34CC4)c3c(Cl)cc(F)cc32)cc1 ; "DAST" ; CC(F)(F)c1ccc(S(=O)(=O)n2nc(N3CCN(c4ncc(F)cn4)CC34CC4)c3c(Cl)cc(F)cc32)cc1]

Step 1: To a solution of 4-chloro-6-fluoro-3-[7-(5-fluoropyrimidin-2-yl)-4,7-diazaspiro[2.5]octan-4-yl]-1H-indazole (0.1 g, 265 μmol, 1 eq) and 4-acetylbenzenesulfonyl chloride (116 mg, 531 μmol, 2 eq) in DCM (1 mL) was added TEA (80.6 mg, 796 μmol, 3 eq) and DMAP (3.24 mg. 26.5 μmol, 0.1 eq) 0° C. The mixture was stirred at 25° C. for 0.5 hour. LC-MS showed 4-chloro-6-fluoro-3-[7-(5-fluoropyrimidin-2-yl)-4, 7-diazaspiro [2.5]octan-4-yl]-1H-indazole was consumed completely and one main peak with desired mass was detected. The residue was diluted with H₂O (10 mL) and extracted with DCM (5 mL*3). The combined organic layers were washed with brine (10 mL), dried over Na₂SO₄, filtered and concentrated under reduced pressure to give a residue. The residue was purified by prep-TLC (Silica gel, petroleum ether/EtOAc=3/1) to give desired 1-[4-[4-chloro-6-fluoro-3-[7-(5-fluoropyrimidin-2-yl)-4, 7-diazaspiro [2.5]octan-4-yl]indazol-1-yl]sulfonylphenyl]ethanone (110 mg, 197 μmol, 74.2% yield) as a yellow solid. MS (ESI): mass calcd. For C₂₅H₂₁N₆ClF₂SO₃ 558.11, mass found 559.1[M+H]⁺.

Step 2: A mixture of 1-[4-[4-chloro-6-fluoro-3-[7-(5-fluoropyrimidin-2-yl)-4,7-diazaspiro[2.5]octan-4-yl]indazol-1-yl]sulfonylphenyl]ethanone (0.1 g, 179 μmol, 1 eq) in DAST (1 mL) at 0° C., and then the mixture was stirred at 70° C. for 0.5 hour. LC-MS showed 1-[4-[4-chloro-6-fluoro-3-[7-(5-fluoropyrimidin-2-yl)-4, 7-diazaspiro [2.5]octan-4-yl]indazol-1-yl]sulfonylphenyl]ethanone was consumed completely and one main peak with desired mass was detected. The residue was diluted with sat.NaHCO₃ (10 mL) and extracted with EtOAc (5 mL*3). The combined organic layers were washed with brine (10 mL), dried over Na₂SO₄, filtered and concentrated under reduced pressure to give a residue. The residue was purified by prep-HPLC (TFA condition; Method: column: Phenomenex Luna C18 75*30 mm*3 μm; mobile phase: [water(TFA)-ACN]; B %: 60%-90%, 8 min) to give desired 4-chloro-1-[4-(1,1-difluoroethyl)phenyl]sulfonyl-6-fluoro-3-[7-(5-fluoropyrimidin-2-yl)-4,7-diazaspiro[2.5]octan-4-yl]indazole (30.2 mg, 52.0 μmol, 29.1% yield, 100.00% purity) as alight yellow solid. ¹H NMR (400 MHz, DMSO-d₆) δ 8.46 (s, 2H), 7.96 (d, J=8.4 Hz, 2H), 7.84 (dd, J=2.1, 8.8 Hz, 1H), 7.72 (d, J=8.5 Hz, 2H), 7.56 (dd, J=2.1, 9.1 Hz, 1H), 3.90 (s, 2H), 3.51 (br s, 4H), 1.91 (t, J=19.1 Hz, 3H), 0.76 (s, 2H), 0.38 (br s, 2H). HPLC: 100.00% (220 nm), 100.00% (215 nm), 100.00% (254 nm). MS (ESI): mass calcd. For C₂₅H₂₁N₆F₄SO₂Cl 580.11, mass found 581.1 [M+H]⁺.

### Compound 92: 4-chloro-1-((4-(cyclopropyldifluoromethyl) phenyl) sulfonyl)-3-(7-(5-fluoropyrimidin-2-yl)-4, 7-diazaspiro [2.5]octan-4-yl)-1H-indazole

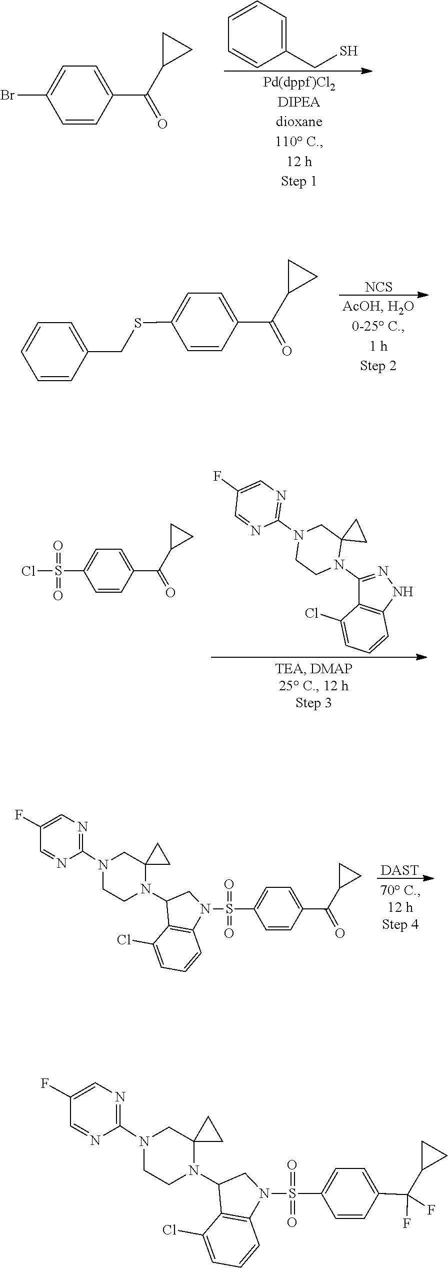

Step 1: A mixture of (4-bromophenyl)-cyclopropyl-methanone (1 g, 4.44 mmol, 1 eq), BnSH (2.93 g, 23.6 mmol, 5.31 eq), Pd(dppf)Cl₂ (195 mg, 267 μmol, 0.06 eq), Xantphos (308 mg, 533 μmol, 0.12 eq) and DIEA (1.15 g, 8.89 mmol, 2 eq) in dioxane (10 mL) was degassed and purged with N₂ for 3 times, and then the mixture was stirred at 110° C. for 12 hours under N₂ atmosphere. TLC showed (4-bromophenyl)-cyclopropyl-methanone was consumed completely. The reaction was cooled down to 15° C. and added water (20 mL). The result mixture was extracted with MTBE (3*20 mL). The combined organics were concentrated to get a residue. The residue was purified by flash silica gel chromatography (ISCO®; 12 g SepaFlash® Silica Flash Column, Eluent of 0˜50% ethyl acetate/petroleum ether gradient @50 mL/min) to give desired (4-benzylsulfanylphenyl)-cyclopropyl-methanone (1.3 g, crude) as a yellow solid.

Step 2: To the solution of (4-benzylsulfanylphenyl)-cyclopropyl-methanone (500 mg, 1.86 mmol, 1 eq) in AcOH (1 mL) and H₂O (0.2 mL) was added NCS (995 mg, 7.45 mmol, 4 eq) at 0° C. and the solution was stirred at 25° C. for 1 hour. LCMS showed (4-benzylsulfanylphenyl)-cyclopropyl-methanone was consumed completely and desired mass was detected (the sample was quenched with piperidine). The reaction was added water (5 mL) and extracted with MTBE (2*20 mL). The combined organics were washed with sodium bicarbonate solution (2*10 mL), dried over anhydrous sodium sulfate and concentrated to get a yellow oil. The residue was purified by flash silica gel chromatography (ISCO®; 12 g SepaFlash® Silica Flash Column, Eluent of 0˜10% ethyl acetate/petroleum ether gradient @50 mL/min) to give desired 4-(cyclopropanecarbonyl) benzenesulfonyl chloride (450 mg, crude) as a yellow oil. MS (ESI): mass calcd. For C₀₁H₉O₃SCl 244.00 mass found 294.0 [M+49+H]⁺.

Step 3: The solution of 4-chloro-3-[7-(5-fluoropyrimidin-2-yl)-4,7-diazaspiro[2.5]octan-4-yl]-1H-indazole (120 mg, 334 μmol, 1 eq), 4-(cyclopropanecarbonyl)benzenesulfonyl chloride (409 mg, 1.67 mmol, 5 eq), TEA (338 mg, 3.34 mmol, 10 eq) and DMAP (4.09 mg, 33.4 μmol, 0.1 eq) in DCM (2 mL) was stirred at 25° C. for 12 hours. LCMS showed 4-chloro-3-[7-(5-fluoropyrimidin-2-yl)-4, 7-diazaspiro [2.5]octan-4-yl]-1H-indazole was consumed completely and desired mass was detected. The reaction was concentrated to get a residue. The residue was purified by prep-TLC (Silica gel, petroleum ether/ethyl acetate=3/1) to give desired [4-[4-chloro-3-[7-(5-fluoropyrimidin-2-yl)-4, 7-diazaspiro [2.5]octan-4-yl]indazol-1-yl]sulfonylphenyl]-cyclopropyl-methanone (170 mg, crude) as a yellow oil. MS (ESI): mass calcd. For C₂₇H₂₄SO₃N₆ClF 566.13 mass found 567.1 [M+H]⁺.

Step 4: The solution of [4-[4-chloro-3-[7-(5-fluoropyrimidin-2-yl)-4,7-diazaspiro[2.5]octan-4-yl]indazol-1-yl]sulfonylphenyl]-cyclopropyl-methanone (120 mg, 212 μmol, 1 eq) in DAST (682 mg, 4.23 mmol, 20 eq) was stirred at 70° C. for 12 hours under the atmosphere of nitrogen. LCMS showed [4-[4-chloro-3-[7-(5-fluoropyrimidin-2-yl)-4, 7-diazaspiro [2.5]octan-4-yl]indazol-1-yl]sulfonylphenyl]-cyclopropyl-methanone remained and desired mass was detected. The reaction was concentrated to get a residue. The reaction was poured into ice-water (20 mL) and extracted with EtOAc (2*10 mL). The combined organics were dried over anhydrous sodium sulfate and concentrated to get a residue. The residue was purified by prep-HPLC (column: Phenomenex Luna C18 75*30 mm*3 μm; mobile phase: [water (TFA)-ACN]; B %: 60%-90%, 8 min) to give desired 4-chloro-1-[4-[cyclopropyl(difluoro)methyl]phenyl]sulfonyl-3-[7-(5-fluoropyrimidin-2-yl)-4,7-diazaspiro[2.5]octan-4-yl]indazole (15.0 mg, 25.5 μmol, 12.0% yield, 100.0% purity) as a gray solid. ¹H NMR (DMSO-d₆) δ 8.46 (s, 2H), 8.08 (d, 1H), 7.89 (d, 2H), 7.71 (d, 2H), 7.64 (m, 1H), 7.47 (d, 1H), 3.91 (s, 2H), 3.46-3.67 (m, 3H), 3.24-3.32 (m, 1H), 1.54-1.69 (m, 1H), 0.76 (br s, 2H), 0.54-0.68 (m, 4H), 0.36 (br s, 2H) HPLC: 100.0% (220 nm), 100.0% (215 nm), 100.0% (254 nm). MS (ESI): mass calcd. For C₂₇H₂₄SO₂N₆ClF₃ 588.13 mass found 589.1 [M+H]⁺.

### Compound 93: 4-chloro-1-[4-(difluoromethylsulfanyl) phenyl]sulfonyl-3-[7-(5-fluoropyrimidin-2-yl)-4, 7-diazaspiro [2.5]octan-4-yl]indazole

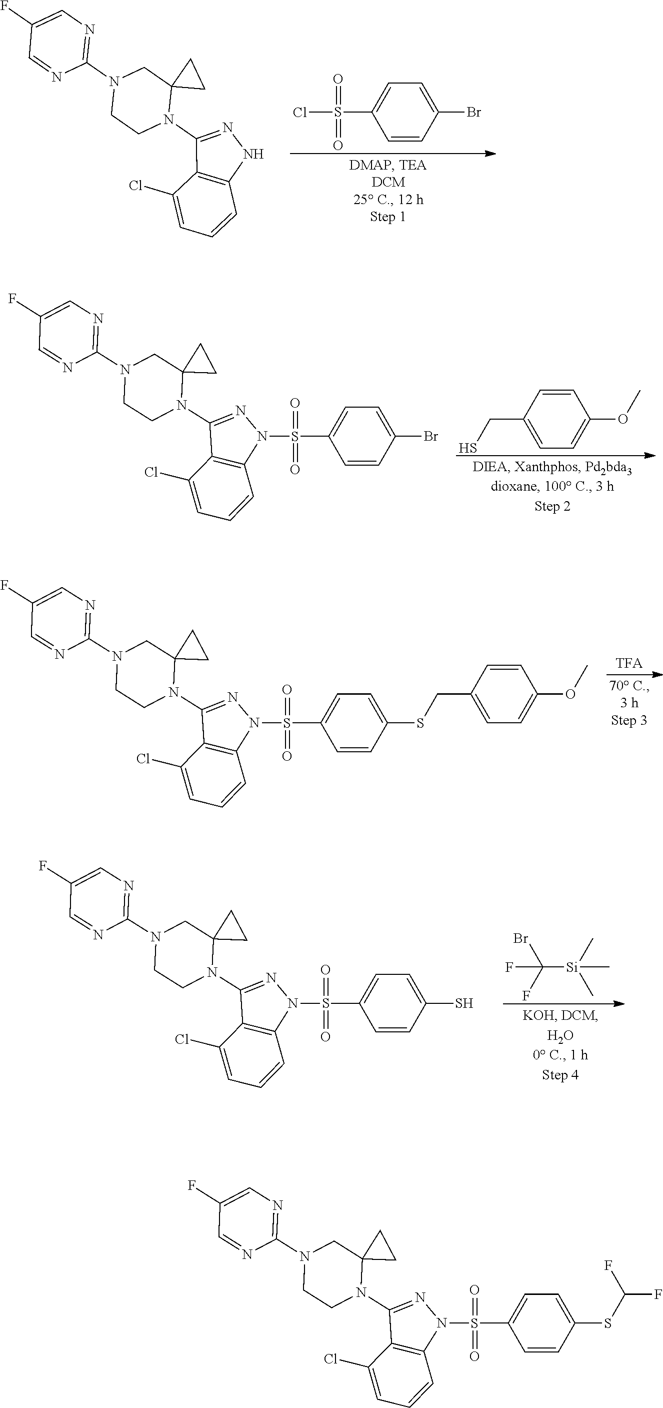

Step 1: To a solution of 4-chloro-3-[7-(5-fluoropyrimidin-2-yl)-4,7-diazaspiro[2.5]octan-4-yl]-1H-indazole (300 mg, 836 μmol, 1 eq) and 4-bromobenzenesulfonyl chloride (428 mg, 1.67 mmol, 2 eq) in DCM (5 mL) was added DMAP (10.2 mg, 83.6 μmol, 0.1 eq) and TEA (254 mg, 2.51 mmol, 349 μL, 3 eq). The mixture was stirred at 25° C. for 12 hours. LCMS showed 4-chloro-3-[7-(5-fluoropyrimidin-2-yl)-4, 7-diazaspiro [2.5]octan-4-yl]-1H-indazole was consumed completely and desired mass was detected. The reaction was concentrated to get a residue. The residue was purified by prep-TLC (Silica gel, petroleum ether/ethyl acetate=3/1) to give desired 1-(4-bromophenyl) sulfonyl-4-chloro-3-[7-(5-fluoropyrimidin-2-yl)-4, 7-diazaspiro [2.5]octan-4-yl]indazole (280 mg, 485 μmol, 58.0% yield) as a yellow solid. MS (ESI): mass calcd. For C₂₃H₁₉BrSO₂N₆ClF 576.01 mass found 577.1[M+H]⁺.

Step 2: A mixture of 1-(4-bromophenyl)sulfonyl-4-chloro-3-[7-(5-fluoropyrimidin-2-yl)-4, 7-diazaspiro [2.5]octan-4-yl]indazole (50 mg, 86.5 μmol, 1 eq), (4-methoxyphenyl)methanethiol (20.0 mg, 130 μmol, 18.0 μL, 1.5 eq), Pd₂(dba)₃ (7.92 mg, 8.65 μmol, 0.1 eq), DIEA (33.6 mg, 260 μmol, 45.2 μL, 3 eq) and Xantphos (2.50 mg, 4.33 μmol, 0.05 eq) in dioxane (2 mL) was degassed and purged with N₂ for 3 times, and then the mixture was stirred at 100° C. for 3 hours under N₂ atmosphere. LC-MS showed 1-(4-bromophenyl) sulfonyl-4-chloro-3-[7-(5-fluoropyrimidin-2-yl)-4, 7-diazaspiro [2.5]octan-4-yl]indazole was consumed completely and one main peak with desired mass was detected. The reaction mixture was concentrated under reduced pressure to give a residue. The residue was purified by prep-TLC (Silica gel, petroleum ether/ethyl acetate=3/1) to give desired 4-chloro-3-[7-(5-fluoropyrimidin-2-yl)-4, 7-diazaspiro [2.5]octan-4-yl]-1-[4-[(4-methoxyphenyl) methylsulfanyl]phenyl]sulfonyl-indazole (50 mg, 76.8 μmol, 88.7% yield) as a yellow oil. MS (ESI): mass calcd. For C₃₁H₂₈S₂O₃N₆ClF 650.13 mass found 651.1[M+H]⁺.

Step 3: To a solution of 4-chloro-3-[7-(5-fluoropyrimidin-2-yl)-4, 7-diazaspiro [2.5]octan-4-yl]-1-[4-[(4-methoxyphenyl) methylsulfanyl]phenyl]sulfonyl-indazole (140 mg, 215 μmol, 1 eq) in TFA (2 mL) was stirred at 70° C. for 3 hours. LC-MS showed 4-chloro-3-[7-(5-fluoropyrimidin-2-yl)-4, 7-diazaspiro [2.5]octan-4-yl]-1-[4-[(4-methoxyphenyl) methylsulfanyl]phenyl]sulfonyl-indazole was consumed completely and one main peak with desired mass was detected. The reaction mixture was concentrated under reduced pressure to give a residue. The residue was purified by prep-TLC (Silica gel, petroleum ether/ethyl acetate=3/1) to give desired 4-[4-chloro-3-[7-(5-fluoropyrimidin-2-yl)-4, 7-diazaspiro [2.5]octan-4-yl]indazol-1-yl]sulfonylbenzenethiol (50 mg, 94.2 μmol, 43.8% yield) as a pale yellow gum. MS (ESI): mass calcd. For C₂₃H₂₀S₂O₂NClF 530.08 mass found 531.1[M+H]⁺.

Step 4: To a solution of 4-[4-chloro-3-[7-(5-fluoropyrimidin-2-yl)-4,7-diazaspiro[2.5]octan-4-yl]indazol-1-yl]sulfonylbenzenethiol (45 mg, 84.7 μmol, 1 eq) in DCM (1 mL) was added KOH (143 mg, 508 μmol, 20% purity, 6 eq) with vigorous stirring. [bromo(difluoro)methyl]-trimethyl-silane (34.4 mg, 169 μmol, 2 eq) in DCM (0.5 mL) was added to the mixture at 0° C. The mixture was stirred at 0° C. for 1 hour. LC-MS showed 4-[4-chloro-3-[7-(5-fluoropyrimidin-2-yl)-4, 7-diazaspiro [2.5]octan-4-yl]indazol-1-yl]sulfonylbenzenethiol was consumed completely and one main peak with desired mass was detected. Then it was partitioned between 5 mL of water and 15 mL of EtOAc. The organic phase was separated, washed with 5 mL of brine, dried over Na₂SO₄, filtered and concentrated under reduced pressure to give a residue. The residue was purified by prep-TLC (Silica gel, petroleum ether/ethyl acetate=3/1) to give desired 4-chloro-1-[4-(difluoromethylsulfanyl) phenyl]sulfonyl-3-[7-(5-fluoropyrimidin-2-yl)-4, 7-diazaspiro [2.5]octan-4-yl]indazole (6.7 mg, 11.0 μmol, 13.0% yield, 95.73% purity) as a white solid. ¹H NMR (400 MHz, DMSO-d₆) δ 8.46 (s, 2H), 8.06 (d, J=8.4 Hz, 1H), 7.83 (d, J=8.4 Hz, 2H), 7.73-7.56 (m, 4H), 7.50-7.43 (m, 1H), 3.92 (s, 2H), 3.61-3.49 (m, 4H), 0.78 (br s, 2H), 0.39 (br s, 2H). HPLC: 89.02% (220 nm), 88.57% (215 nm), 95.73% (254 nm). MS (ESI): mass calcd. For C₂₄H₂₀S₂O₂N₆ClF₃ 580.07 mass found 581.1[M+H]⁺.

### Compound 94: 1-((4-(tert-butyl) phenyl) sulfonyl)-4-chloro-3-(7-(5-fluoropyrimidin-2-yl)-4, 7-diazaspiro [2.5]octan-4-yl)-1H-indazole

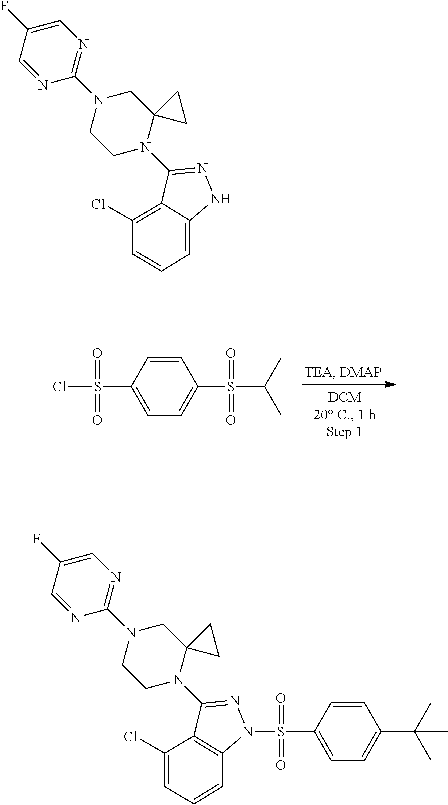 [SMILES: Fc1cnc(N2CCN(c3n[nH]c4cccc(Cl)c34)C3(CC3)C2)nc1 ; CC(C)S(=O)(=O)c1ccc(S(=O)(=O)Cl)cc1 ; CC(C)(C)c1ccc(S(=O)(=O)n2nc(N3CCN(c4ncc(F)cn4)CC34CC4)c3c(Cl)cccc32)cc1]

Step 1: To a solution of 4-chloro-3-[7-(5-fluoropyrimidin-2-yl)-4,7-diazaspiro[2.5]octan-4-yl]-1H-indazole (30.0 mg, 83.6 μmol, 1 eq) and 4-tert-butylbenzenesulfonyl chloride (38.9 mg, 167 μmol, 2 eq) in DCM (1 mL) was added TEA (16.9 mg, 167 μmol, 23.3 μL, 2 eq) and DMAP (1.02 mg, 8.36 μmol, 0.1 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole was consumed completely and one main peak with desired mass was detected. The reaction mixture was added to water (10 mL), extracted with EtOAc (10 mL*3). The combined organic layers were washed with brine (20 mL) and dried over Na₂SO₄, filtered and concentrated under reduced pressure to give a residue. The residue was purified by prep-TLC (Silica gel, petroleum ether/ethyl acetate=3/1) to give desired 1-(4-tert-butylphenyl) sulfonyl-4-chloro-3-[7-(5-fluoropyrimidin-2-yl)-4, 7-diazaspiro [2.5]octan-4-yl]indazole (20 mg, 34.5 μmol, 41.3% yield, 95.91% purity) as a white solid. ¹H NMR (400 MHz, DMSO-d₆) δ 8.49-8.43 (m, 2H), 8.10-8.04 (m, 1H), 7.72-7.58 (m, 3H), 7.56-7.49 (m, 2H), 7.47-7.41 (m, 1H), 3.94-3.85 (m, 2H), 3.51 (br s, 4H), 1.20-1.14 (m, 9H), 0.79-0.69 (m, 2H), 0.44-0.30 (m, 2H). HPLC: 95.55% (220 nm), 95.71% (215 nm), 95.91% (254 nm). MS (ESI): mass calcd. For C₂₇H₂₈N₆SO₂ClF 554.17 mass found 555.1 [M+H]⁺.

### Compound 95: 4-chloro-3-(7-(5-fluoropyrimidin-2-yl)-4, 7-diazaspiro [2.5]octan-4-yl)-1-((4-(1, 1, 2, 2-tetrafluoroethyl) phenyl) sulfonyl)-1H-indazole

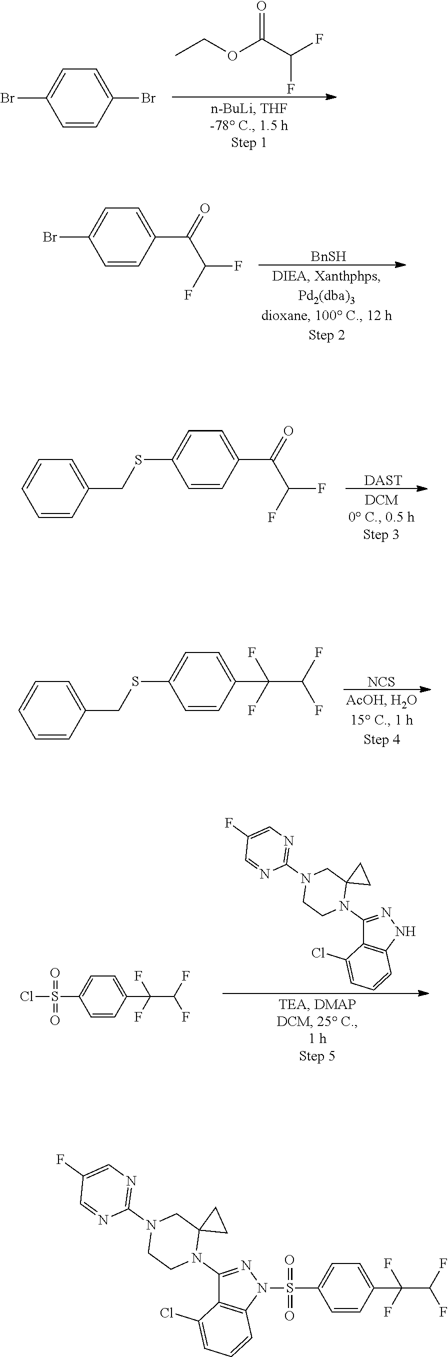

Step 1: 1, 4-dibromobenzene (5 g, 21.2 mmol, 2.72 mL, 1 eq) was dissolved in THF (50 mL). The reaction solution was cooled to −78° C., and n-BuLi (2.5 M, 8.90 mL, 1.05 eq) was added dropwise to the reaction solution. After the dropwise addition, the reaction solution was stirred at −78° C. for 0.5 hour under N₂. To the reaction solution was added ethyl 2, 2-difluoroacetate (5.79 g, 46.6 mmol, 2.2 eq). After the addition, the reaction solution was stirred at −78° C. for 1 hour under N₂. TLC showed 1, 4-dibromobenzene was consumed completely and desired mass was detected. Then it was separated between sat. NH₄Cl (50 mL) and ethyl acetate (30 mL). The organic phase was separated, washed with brine (60 mL), dried over Na₂SO₄, filtered and concentrated under reduced pressure to give the crude product. The residue was purified by chromatography give desired 1-(4-bromophenyl)-2, 2-difluoroethan-1-one (4.69 g, 20.0 mmol, 94.2% yield) as a yellow oil.

Step 2: A mixture of 1-(4-bromophenyl)-2,2-difluoro-ethanone (4.69 g, 20.0 mmol, 1 eq), phenylmethanethiol (2.97 g, 24.0 mmol, 2.81 mL, 1.2 eq), DIEA (5.16 g, 39.9 mmol, 6.95 mL, 2 eq), Xantphos (2.31 g, 3.99 mmol, 0.2 eq) and Pd₂(dba)₃ (1.83 g, 2.00 mmol, 0.1 eq) in dioxane (50 mL) was degassed and purged with N₂ for 3 times, and then the mixture was stirred at 100° C. for 12 hours under N₂ atmosphere. TLC (Silica gel, petroleum ether/ethyl acetate=5/1) showed 1-(4-bromophenyl)-2, 2-difluoro-ethanone was consumed completely and desired mass was detected. Then it was separated between sat. NH₄Cl (50 mL) and EtOAc (80 mL). The organic phase was separated, washed with brine (50 mL), dried over Na₂SO₄, filtered and concentrated under reduced pressure to give the crude product. The residue was purified by chromatography give desired 1-(4-(benzylthio) phenyl)-2, 2-difluoroethan-1-one (2 g, 7.19 mmol, 36.0% yield) as a yellow solid.

Step 3: To a solution of 1-(4-benzylsulfanylphenyl)-2, 2-difluoro-ethanone (1.2 g, 4.31 mmol, 1 eq) in DCM (12 mL) was added DAST (3.47 g, 21.6 mmol, 2.85 mL, 5 eq). The mixture was stirred at 0° C. for 0.5 hour. TLC (Silica gel, petroleum ether/ethyl acetate=2/1) showed 1-(4-bromophenyl)-2, 2-difluoro-ethanone was consumed completely and desired mass was detected. Then it was separated between sat. NH₄Cl (50 mL) and EtOAc (80 mL). The organic phase was separated, washed with brine (50 mL), dried over Na₂SO₄, filtered and concentrated under reduced pressure to give desired benzyl(4-(1, 1, 2, 2-tetrafluoroethyl) phenyl) sulfane (1.29 g, crude) as a yellow solid.

Step 4: To a solution of 1-benzylsulfanyl-4-(1, 1, 2, 2-tetrafluoroethyl) benzene (200 mg, 666 μmol, 1 eq) in AcOH (1 mL) and H₂O (0.3 mL) was added NCS (356 mg, 2.66 mmol, 4 eq). The mixture was stirred at 15° C. for 1 hour. LC-MS showed 1-benzylsulfanyl-4-(1, 1, 2, 2-tetrafluoroethyl) benzene was consumed completely and desired mass was detected. Then it was separated between sat. NH₄Cl (10 mL) and EtOAc (10 mL). The organic phase was separated, washed with brine (50 mL), dried over Na₂SO₄, filtered and concentrated under reduced pressure to give desired 4-(1,1,2,2-tetrafluoroethyl)benzenesulfonyl chloride (184 mg, crude) as a white solid. MS (ESI): mass calcd. For C₈H₅ClF₄O₂S 275.96 mass found 326.0 [M+H+49]⁺.

Step 5: To a solution of 4-chloro-3-[7-(5-fluoropyrimidin-2-yl)-4,7-diazaspiro[2.5]octan-4-yl]-1H-indazole (30 mg, 83.6 μmol, 1 eq) and 4-(1,1,2,2-tetrafluoroethyl) benzenesulfonyl chloride (69.4 mg, 251 μmol, 3 eq) in DCM (1 mL) was added TEA (16.9 mg, 167 μmol, 23.3 μL, 2 eq) and DMAP (1.02 mg, 8.36 μmol, 0.1 eq). The mixture was stirred at 25° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was filtered and the filtrate was concentrated under reduced pressure to give a residue. The residue was purified by prep-HPLC (TFA condition, column: Phenomenex Luna C18 75*30 mm*3 μm; mobile phase: [water(TFA)-ACN]; B %: 60%-90%, 8 min) give desired 4-chloro-3-(7-(5-fluoropyrimidin-2-yl)-4,7-diazaspiro[2.5]octan-4-yl)-1-((4-(1,1,2,2-tetrafluoroethyl) phenyl)sulfonyl)-1H-indazole (15 mg, 24.7 μmol, 29.5% yield, 98.63% purity) as a white solid. ¹H NMR (400 MHz, DMSO-d₆) δ 8.46 (s, 2H), 8.12-7.95 (m, 3H), 7.78 (d, J=8.5 Hz, 2H), 7.65 (t, J=8.1 Hz, 1H), 7.47 (d, J=7.6 Hz, 1H), 6.90-6.58 (m, 1H), 3.92 (s, 2H), 3.60 (br s, 2H), 3.53 (br s, 2H), 0.75 (s, 2H), 0.34 (br s, 2H). HPLC: 98.63% (220 nm), 98.74% (215 nm), 98.80 (254 nm). MS (ESI): mass calcd. For C₂₅H₂₀ClF₅N₆O₂S 598.10 mass found 599.1 [M+H]⁺.

### Compound 96: 4-chloro-1-[4-(2-fluoro-1, 1-dimethyl-ethyl) phenyl]sulfonyl-3-[7-(5-fluoropyrimidin-2-yl)-4, 7-diazaspiro [2.5]octan-4-yl]indazole

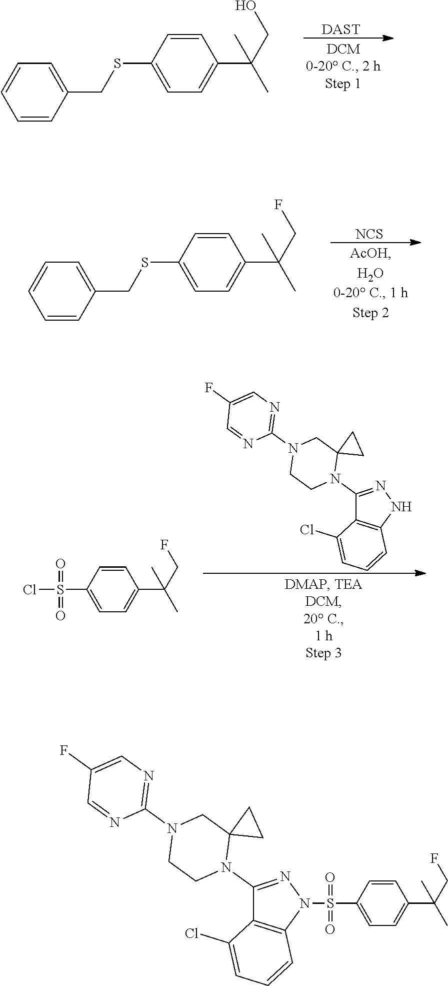

Step 1: To a solution of 3-(4-benzylsulfanylphenyl)-3-methyl-butan-1-ol (220 mg, 768 μmol, 1 eq) in DCM (2 mL) was added DAST (136 mg, 845 μmol, 112 μL, 1.1 eq) at 0° C. The mixture was stirred at 20° C. for 2 hours. TLC indicated 3-(4-benzylsulfanylphenyl)-3-methyl-butan-1-ol was consumed, and one major new spot with lower polarity was detected. The reaction mixture was quenched by sat. aq. NaHCO₃ (2 mL) and extracted with DCM (5 mL), washed with 5 mL of brine, dried over Na₂SO₄, filtered and concentrated under reduced pressure to give a residue. The residue was purified by prep-TLC (Silica gel, petroleum ether/ethyl acetate=5/1) to give desired 1-benzylsulfanyl-4-(3-fluoro-1, 1-dimethyl-propyl) benzene (90 mg, 312 μmol, 40.6% yield) as a pale yellow oil.

Step 2: To a solution of 1-benzylsulfanyl-4-(2-fluoro-1, 1-dimethyl-ethyl) benzene (90.0 mg, 328 μmol, 1 eq) in AcOH (1 mL) and H₂O (0.2 mL) was added NCS (131 mg, 984 μmol, 3 eq) at 0° C. The mixture was stirred at 20° C. for 1 hour. TLC indicated 1-benzylsulfanyl-4-(2-fluoro-1, 1-dimethyl-ethyl) benzene was consumed completely, and one major new spot with larger polarity was detected. The reaction mixture was concentrated under reduced pressure to give a residue. The residue was purified by prep-TLC (Silica gel, petroleum ether/ethyl acetate=5/1) to give desired 4-(2-fluoro-1, 1-dimethyl-ethyl) benzenesulfonyl chloride (70 mg, 279 μmol, 85.1% yield) as a white gum.

Step 3: To a solution of 4-chloro-3-[7-(5-fluoropyrimidin-2-yl)-4, 7-diazaspiro [2.5]octan-4-yl]-1H-indazole (50 mg, 139 μmol, 1 eq), 4-(2-fluoro-1, 1-dimethyl-ethyl) benzenesulfonyl chloride (63.0 mg, 251 μmol, 1.8 eq) and TEA (42.3 mg, 418 μmol, 58.2 μL, 3 eq) in DCM (0.5 mL) was added DMAP (17.0 mg, 139 μmol, 1 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole remained and one main peak with desired mass was detected. The mixture was concentrated under reduced pressure to give a residue. The residue was purified by prep-TLC (Silica gel, petroleum ether/ethyl acetate=3/1) to give desired 4-chloro-1-[4-(2-fluoro-1, 1-dimethyl-ethyl) phenyl]sulfonyl-3-[7-(5-fluoropyrimidin-2-yl)-4, 7-diazaspiro [2.5]octan-4-yl]indazole (31.2 mg, 53.9 μmol, 38.7% yield, 99.08% purity) as a white solid. ¹H NMR (400 MHz, DMSO-d₆) δ 8.46 (s, 2H), 8.07 (d, J=8.4 Hz, 1H), 7.72 (d, J=8.4 Hz, 2H), 7.62 (t, J=8.1 Hz, 1H), 7.44 (d, J=7.6 Hz, 1H), 7.35 (d, J=8.3 Hz, 2H), 3.91 (s, 2H), 3.67-3.47 (m, 4H), 2.97-2.80 (m, 2H), 1.22-1.11 (m, 6H), 0.75 (br s, 2H), 0.37 (br s, 2H). HPLC: 95.95% (220 nm), 95.99% (215 nm), 99.08% (254 nm). MS (ESI): mass calcd. For C₂₇H₂₇N₆SO₂ClF₂ 572.16 mass found 573.0[M+H]⁺.

### Compound 97: 4-chloro-1-((4-(2-fluoropropan-2-yl) phenyl) sulfonyl)-3-(7-(5-fluoropyrimidin-2-yl)-4, 7-diazaspiro [2.5]octan-4-yl)-1H-indazole

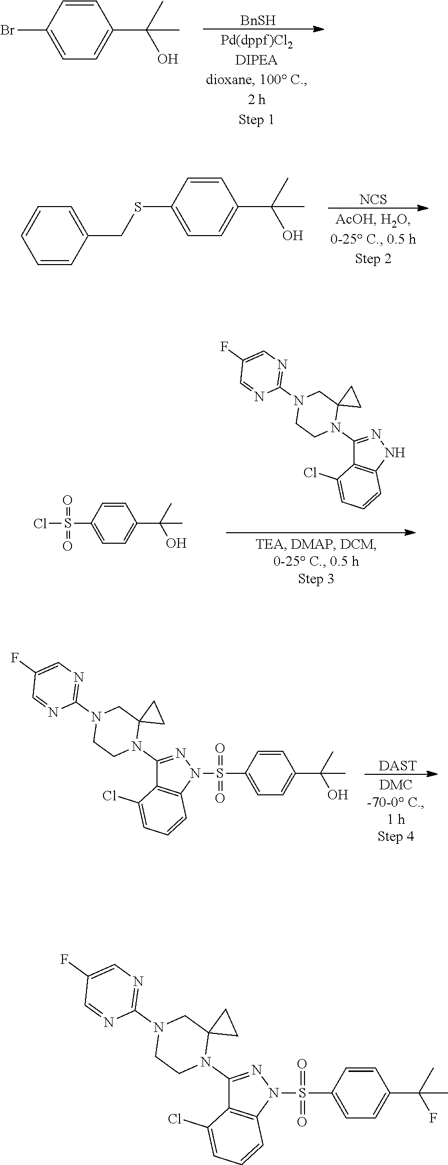

Step 1: To a solution of 2-(4-bromophenyl) propan-2-ol (2 g, 9.30 mmol, 1 eq) and BnSH (1.50 g, 12.1 mmol, 1.42 mL, 1.3 eq) in dioxane (20 mL) was added DIEA (2.40 g, 18.6 mmol, 3.24 mL, 2 eq), Xantphos (538 mg, 930 μmol, 0.1 eq) and Pd(dppf)Cl₂ (340 mg, 465 μmol, 0.05 eq) the mixture was stirred at 100° C. for 2 hours under N₂. TLC (petroleum ether/EtOAc=5/1) indicated 2-(4-bromophenyl) propan-2-ol was consumed completely and two new spots formed. The residue was diluted with H₂O (100 mL) and extracted with ethyl acetate (50 mL*3). The combined organic layers were washed with brine (100 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜10% EtOAc/petroleum ether gradient @100 mL/min) to give desired 2-(4-benzylsulfanylphenyl) propan-2-ol (2 g, 7.74 mmol, 83.3% yield) as a yellow oil.

Step 2: To a solution of 2-(4-benzylsulfanylphenyl) propan-2-ol (0.5 g, 1.94 mmol, 1 eq) in AcOH (4 mL) and H₂O (1 mL) was added NCS (775 mg, 5.81 mmol, 3 eq) at 0° C. The mixture was stirred at 25° C. for 0.5 hour. TLC (petroleum ether/EtOAc=5/1) indicated 2-(4-benzylsulfanylphenyl) propan-2-ol was consumed completely and two new spots formed. The residue was diluted with H₂O (10 mL) and extracted with MTBE (5 mL*3). The combined organic layers were washed with brine (10 mL), dried over Na₂SO₄, filtered and concentrated under reduced pressure to give a residue. The residue was purified by prep-TLC (Silica gel, petroleum ether/EtOAc=5/1) to give desired 4-(1-hydroxy-1-methyl-ethyl) benzenesulfonyl chloride (230 mg, 980 μmol, 50.6% yield) as a yellow oil.

Step 3: To a solution of 4-chloro-3-[7-(5-fluoropyrimidin-2-yl)-4, 7-diazaspiro[2.5]octan-4-yl]-1H-indazole (60 mg, 167 μmol, 1 eq) and 4-(1-hydroxy-1-methyl-ethyl) benzenesulfonyl chloride (196 mg, 836 μmol, 5 eq) in DCM (1 mL) was added TEA (50.8 mg, 502 μmol, 3 eq) and DMAP (2.04 mg, 16.7 μmol, 0.1 eq) 0° C. The mixture was stirred at 25° C. for 0.5 hour. LC-MS showed 4-chloro-3-[7-(5-fluoropyrimidin-2-yl)-4, 7-diazaspiro [2.5]octan-4-yl]-1H-indazole was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-TLC (Silica gel, petroleum ether/EtOAc=2/1) to give desired 2-[4-[4-chloro-3-[7-(5-fluoropyrimidin-2-yl)-4, 7-diazaspiro [2.5]octan-4-yl]indazol-1-yl]sulfonylphenyl]propan-2-ol (60 mg, 108 μmol, 64.4% yield) as a yellow solid. MS (ESI): mass calcd. For C₂₆H₂₆N₆ClFSO₃ 556.15, mass found 557.1[M+H]⁺.

Step 4: To a solution of 2-[4-[4-chloro-3-[7-(5-fluoropyrimidin-2-yl)-4,7-diazaspiro[2.5]octan-4-yl]indazol-1-yl]sulfonylphenyl]propan-2-ol (60 mg, 108 μmol, 1 eq) in DCM (1 mL) was added DAST (52.1 mg, 323 μmol, 3 eq) at −70° C. The mixture was stirred at 0° C. for 1 hour. LC-MS showed 2-[4-[4-chloro-3-[7-(5-fluoropyrimidin-2-yl)-4, 7-diazaspiro [2.5]octan-4-yl]indazol-1-yl]sulfonylphenyl]propan-2-ol was consumed completely and one main peak with desired mass was detected. The reaction mixture was quenched by addition sat.NaHCO₃ (10 mL) at 0° C., and then diluted with H₂O (10 mL) and extracted with EtOAc (15 mL*2). The combined organic layers were washed with brine (10 mL), dried over Na₂SO₄, filtered and concentrated under reduced pressure to give a residue. The residue was purified by prep-HPLC (TFA condition; Method: column: Phenomenex Luna C18 75*30 mm*3 μm; mobile phase: [water(TFA)-ACN]; B %: 60%-90%, 8 min) to give 4-chloro-1-[4-(1-fluoro-1-methyl-ethyl)phenyl]sulfonyl-3-[7-(5-fluoropyrimidin-2-yl)-4,7-diazaspiro[2.5]octan-4-yl]indazole (19.8 mg, 35.4 μmol, 32.9% yield, 100.00% purity) as a white solid. ¹H NMR (400 MHz, DMSO-d₆) δ 8.46 (d, J=0.7 Hz, 2H), 8.08 (d, J=7.9 Hz, 1H), 7.77 (d, J=8.3 Hz, 2H), 7.63 (t, J=8.1 Hz, 1H), 7.53 (d, J=8.6 Hz, 2H), 7.48-7.43 (m, 1H), 3.95-3.86 (m, 2H), 3.52 (br s, 4H), 1.57 (s, 3H), 1.52 (s, 3H), 0.80-0.72 (m, 2H), 0.41-0.34 (m, 2H). HPLC: 100.00% (220 nm), 100.00% (215 nm), 100.00% (254 nm). MS (ESI): mass calcd. For C₂₆H₂₅N₆F₂SO₂Cl 558.14, mass found 559.2[M+H]⁺.

### Compound 98: 4-chloro-3-[7-(5-fluoropyrimidin-2-yl)-4, 7-diazaspiro [2.5]octan-4-yl]-1-[4-(1, 1, 2-trifluoroethyl) phenyl]sulfonyl-indazole

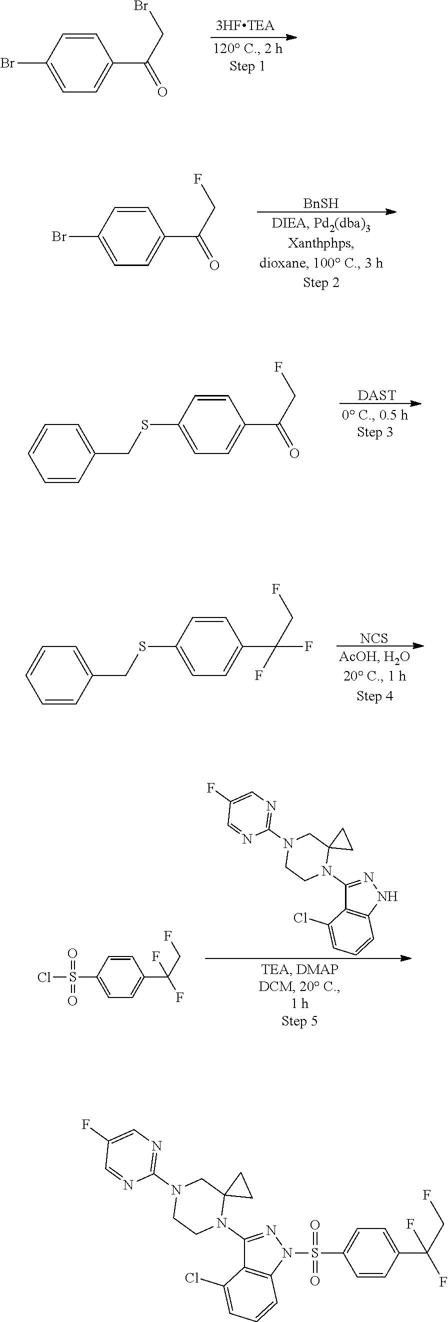

Step 1: To a solution of 2-bromo-1-(4-bromophenyl) ethanone (3 g, 10.8 mmol, 1 eq) was added 3HF·TEA (19.8 g, 123 mmol, 20 mL, 11.4 eq). The mixture was stirred at 120° C. for 2 hours. TLC (petroleum ether/ethyl acetate=10/1) indicated 2-bromo-1-(4-bromophenyl) ethanone was consumed completely and one new spot formed. The reaction was clean according to TLC. The reaction mixture was added to ice water (20 mL), extracted with EtOAc (10 mL*3). The combined organic layers were washed with brine 20 mL and dried over Na₂SO₄, filtered and concentrated under reduced pressure to give a residue. The residue was purified by column chromatography (Silica gel, petroleum ether/ethyl acetate=1/0 to 5/1) to give desired 1-(4-bromophenyl)-2-fluoro-ethanone (1.8 g, crude) as a white solid.

Step 2: A mixture of phenylmethanethiol (504 mg, 4.05 mmol, 475 μL, 1.1 eq), 1-(4-bromophenyl)-2-fluoro-ethanone (800 mg, 3.69 mmol, 1 eq), DIEA (953 mg, 7.37 mmol, 1.28 mL, 2 eq), Xantphos (213 mg, 369 μmol, 0.1 eq) and Pd₂(dba)₃ (84.4 mg, 92.2 μmol, 0.025 eq) in dioxane (10 mL) was degassed and purged with N₂ for 3 times, and then the mixture was stirred at 100° C. for 3 hours under N₂ atmosphere. TLC (petroleum ether/ethyl acetate=5/1) indicated 1-(4-bromophenyl)-2-fluoro-ethanone was consumed completely and one new spot formed. The reaction was clean according to TLC. The reaction mixture was added to water (20 mL), extracted with EtOAc (20 mL*3). The combined organic layers were washed with brine 20 mL and dried over Na₂SO₄, filtered and concentrated under reduced pressure to give a residue. The residue was purified by column chromatography (Silica gel, petroleum ether/ethyl acetate=1/0 to 1/1) to give desired 1-(4-benzylsulfanylphenyl)-2-fluoro-ethanone (840 mg, crude) as a yellow solid.

Step 3: To a solution of 1-(4-benzylsulfanylphenyl)-2-fluoro-ethanone (540 mg, 2.07 mmol, 1 eq) in DCM (3 mL) was added DAST (1.67 g, 10.4 mmol, 1.37 mL, 5 eq) at 0° C. The mixture was stirred at 0° C. for 0.5 hour. TLC (petroleum ether/ethyl acetate=5/1) indicated 1-(4-benzylsulfanylphenyl)-2-fluoro-ethanone was consumed completely and one new spot formed. The reaction was clean according to TLC. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by column chromatography (Silica gel, petroleum ether/ethyl acetate=1/0 to 1/1) to give desired 1-benzylsulfanyl-4-(1, 1, 2-trifluoroethyl) benzene (400 mg, crude) as a colourless oil.

Step 4: To a solution of 1-benzylsulfanyl-4-(1, 1, 2-trifluoroethyl) benzene (400 mg, 1.42 mmol, 1 eq) in AcOH (2 mL) and H₂O (0.4 mL) was added NCS (568 mg, 4.25 mmol, 3 eq). The mixture was stirred at 20° C. for 1 hour. LC-MS showed 1-benzylsulfanyl-4-(1, 1, 2-trifluoroethyl) benzene was consumed completely and desired compound was detected. (The sample was quenched with pyrrolidine) The reaction mixture was diluted with water (10 mL) and extracted with EtOAc (10 mL*3). The combined organic layers were washed with brine 10 mL, dried over anhydrous Na₂SO₄, filtered and concentrated under reduced pressure to give a residue. The residue was purified by prep-TLC (Silica gel, petroleum ether/ethyl acetate=5/1) to give desired 4-(1, 1, 2-trifluoroethyl) benzenesulfonyl chloride (360 mg, crude) as a colourless oil. MS (ESI): mass calcd. For C₈H₆ClF₃O₂S 257.97, mass found 294.0 [M+H+35]⁺.

Step 5: To a solution of 4-chloro-3-[7-(5-fluoropyrimidin-2-yl)-4,7-diazaspiro[2.5]octan-4-yl]-1H-indazole (50 mg, 139 μmol, 1 eq) and 4-(1, 1, 2-trifluoroethyl) benzenesulfonyl chloride (144 mg, 557 μmol, 4 eq) in DCM (2 mL) was added dropwise TEA (70.5 mg, 697 μmol, 97.0 uL, 5 eq) and DMAP (1.70 mg, 13.9 μmol, 0.1 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was concentrated to give the crude product. The residue was purified by prep-HPLC (TFA condition; column: Phenomenex Luna C18 75*30 mm*3 μm; mobile phase: [water(TFA)-ACN]; B %: 50%-80%, 8 min) to give desired 4-chloro-3-[7-(5-fluoropyrimidin-2-yl)-4, 7-diazaspiro [2.5]octan-4-yl]-1-[4-(1, 1, 2-trifluoroethyl) phenyl]sulfonyl-indazole (6.4 mg, 11.0 μmol, 7.87% yield, 99.56% purity) as a yellow solid. ¹H NMR (400 MHz, DMSO-d₆) δ 8.46 (s, 2H), 8.16 (s, 4H), 8.09 (d, J=8.5 Hz, 1H), 7.68 (t, J=8.1 Hz, 1H), 7.52-7.47 (m, 1H), 7.37-7.20 (m, 1H), 3.92 (s, 2H), 3.71-3.57 (m, 2H), 3.55 (br s, 2H), 0.76 (s, 2H), 0.34 (br s, 2H). HPLC: 99.56% (220 nm), 99.58% (210 nm), 99.34% (254 nm). MS (ESI): mass calcd. For C₂₅H₂₁ClF₄N₆O₂S 580.11, mass found 581.1 [M+H]⁺.

### Compound 99: 4-chloro-1-[4-(1, 1-difluoropropyl) phenyl]sulfonyl-3-[7-(5-fluoropyrimidin-2-yl)-4, 7-diazaspiro [2.5]octan-4-yl]indazole

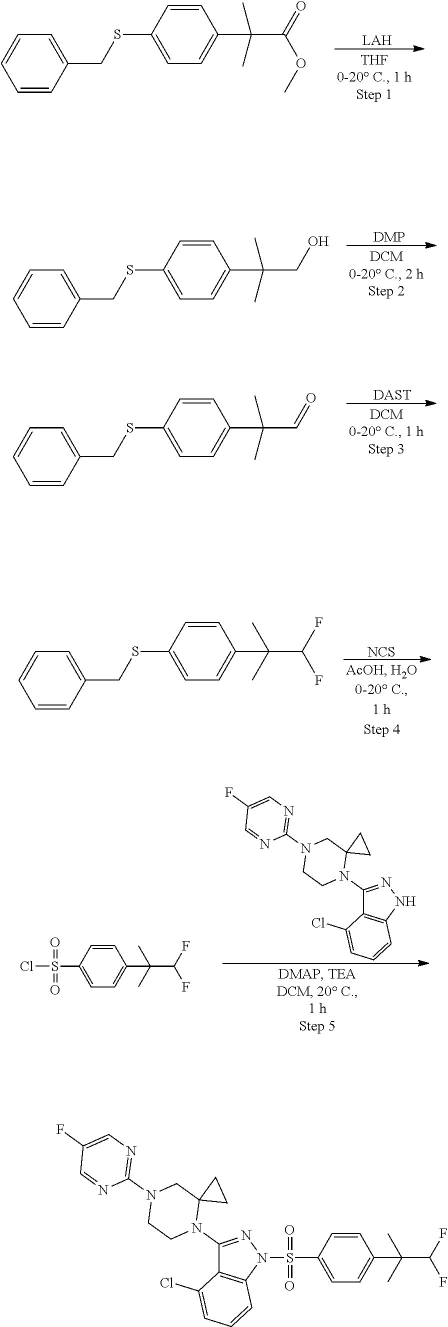

Step 1: To a solution of methyl 2-(4-benzylsulfanylphenyl)-2-methyl-propanoate (250 mg, 832 μmol, 1 eq) in THF (5 mL) was added LAH (94.8 mg, 2.50 mmol, 3 eq) at 0° C. under N₂. The mixture was stirred at 20° C. for 1 hour under N₂ atmosphere. TLC indicated methyl 2-(4-benzylsulfanylphenyl)-2-methyl-propanoate was consumed completely, and one major new spot with larger polarity was detected. The reaction mixture was added dropwise 0.08 mL of water at 0° C. and then it was added dropwise 0.08 mL of 15% aq.NaOH at 0° C. Then it was added dropwise 0.24 mL of water at 0° C. The mixture was dried over anhydrous sodium sulfate, filtered and concentrated under reduced pressure to give a residue. The residue was purified by prep-TLC (Silica gel, petroleum ether/ethyl acetate=5/1) to give desired 2-(4-benzylsulfanylphenyl)-2-methyl-propan-1-ol (200 mg, 734 μmol, 88.2% yield) as a colorless oil.

Step 2: To a solution of 2-(4-benzylsulfanylphenyl)-2-methyl-propan-1-ol (200 mg, 734 μmol, 1 eq) in DCM (2 mL) was added DMP (343 mg, 808 μmol, 250 μL, 1.1 eq) at 0° C. The mixture was stirred at 20° C. for 2 hours. TLC indicated 2-(4-benzylsulfanylphenyl)-2-methyl-propan-1-ol remained, and one major new spot with lower polarity was detected. The mixture quenched by sat.aq.Na₂SO₃ (3 mL) and extracted with ethyl acetate (5 mL), washed with 5 mL of brine, dried over Na₂SO₄, filtered and concentrated under reduced pressure to give a residue. The residue was purified by prep-TLC (Silica gel, petroleum ether/ethyl acetate=5/1) to give desired 2-(4-benzylsulfanylphenyl)-2-methyl-propanal (60 mg, 222 μmol, 30.2% yield) as a colorless oil.

Step 3: To a solution of 2-(4-benzylsulfanylphenyl)-2-methyl-propanal (60 mg, 222 μmol, 1 eq) in DCM (1 mL) was added DAST (71.5 mg, 444 μmol, 58.6 uL, 2 eq) at 0° C. The mixture was stirred at 20° C. for 1 hour. TLC indicated 2-(4-benzylsulfanylphenyl)-2-methyl-propanal was consumed, and one major new spot with lower polarity was detected. The reaction mixture was quenched by sat. aq. NaHCO₃ (1 mL) and extracted with DCM(5 mL), washed with 5 mL of brine, dried over Na₂SO₄, filtered and concentrated under reduced pressure to give a residue. The residue was purified by prep-TLC (Silica gel, petroleum ether/ethyl acetate=10/1) to give desired 1-benzylsulfanyl-4-(2, 2-difluoro-1, 1-dimethyl-ethyl) benzene (50 mg, 171 μmol, 77.1% yield) as a colorless oil.

Step 4: To a solution of 1-benzylsulfanyl-4-(2, 2-difluoro-1, 1-dimethyl-ethyl) benzene (50 mg, 171 μmol, 1 eq) in AcOH (0.5 mL) and H₂O (0.1 mL) was added NCS (68.5 mg, 513 μmol, 3 eq) at 0° C. The mixture was stirred at 20° C. for 1 hour. TLC indicated 1-benzylsulfanyl-4-(2, 2-difluoro-1, 1-dimethyl-ethyl) benzene was consumed completely, and one major new spot with larger polarity was detected. The reaction mixture was concentrated under reduced pressure to give a residue. The residue was purified by prep-TLC (Silica gel, petroleum ether/ethyl acetate=10/1) to give 4-(2, 2-difluoro-1, 1-dimethyl-ethyl) benzenesulfonyl chloride (40 mg, 149 μmol, 87.1% yield) as a pale yellow oil.

Step 5: To a solution of 4-chloro-3-[7-(5-fluoropyrimidin-2-yl)-4,7-diazaspiro[2.5]octan-4-yl]-1H-indazole (30 mg, 83.6 μmol, 1 eq), 4-(2,2-difluoro-1,1-dimethyl-ethyl)benzenesulfonyl chloride (44.9 mg, 167 μmol, 2 eq) and DMAP (10.2 mg, 83.6 μmol, 1 eq) in DCM (1 mL) was added TEA (25.4 mg, 251 μmol, 34.9 μL, 3 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole remained and one main peak with desired mass was detected. The reaction mixture was concentrated under reduced pressure to give a residue. The residue was purified by prep-HPLC (TFA condition; column: Phenomenex Luna C18 75*30 mm*3 μm; mobile phase: [water(TFA)-ACN]; B %: 50%-80%, 8 min) to give desired 4-chloro-1-[4-(2,2-difluoro-1,1-dimethyl-ethyl)phenyl]sulfonyl-3-[7-(5-fluoropyrimidin-2-yl)-4,7-diazaspiro[2.5]octan-4-yl]indazole (7.1 mg, 12.0 μmol, 14.3% yield, 99.50% purity) as a white solid. ¹H NMR (400 MHz, DMSO-d₆) δ 8.46 (s, 2H), 8.08 (br d, J=8.3 Hz, 1H), 7.84 (br d, J=7.6 Hz, 2H), 7.69-7.40 (m, 4H), 5.76-5.46 (m, 1H), 3.92 (br s, 2H), 3.65-3.49 (m, 4H), 1.36-1.05 (m, 6H), 0.75 (br s, 2H), 0.35 (br s, 2H). HPLC: 99.11% (220 nm), 99.17% (215 nm), 99.50% (254 nm). MS (ESI): mass calcd. For C₂₇H₂₆N₆SO₂ClF₃ 590.15 mass found 591.1[M+H]⁺.

### Compound 100: 1-(2-tert-butylpyrimidin-5-yl) sulfonyl-4-chloro-3-[7-(5-fluoropyrimidin-2-yl)-4, 7-diazaspiro [2.5]octan-4-yl]indazole

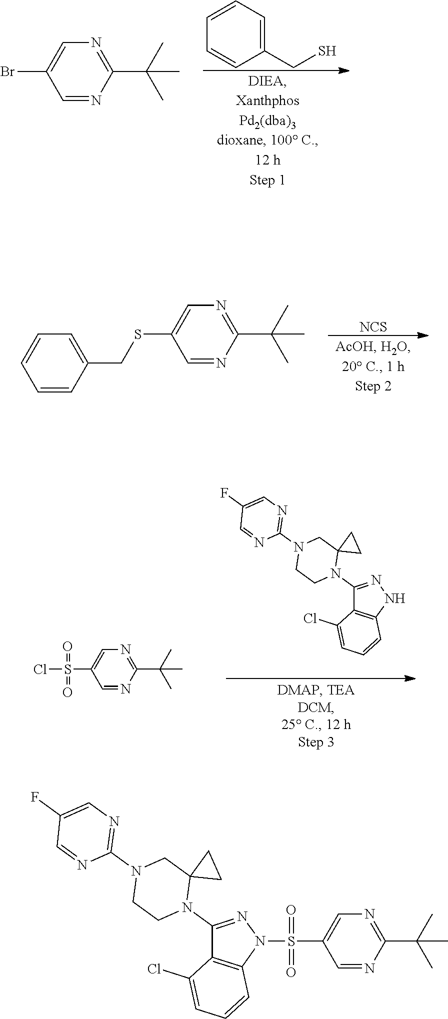

Step 1: A mixture of 5-bromo-2-tert-butyl-pyrimidine (700 mg, 3.25 mmol, 1 eq), phenylmethanethiol (485 mg, 3.91 mmol, 458 μL, 1.2 eq), DIEA (841 mg, 6.51 mmol, 1.13 mL, 2 eq), Xantphos (377 mg, 651 μmol, 0.2 eq) and Pd₂(dba)₃ (298 mg, 325 μmol, 0.1 eq) in dioxane (3 mL) was degassed and purged with N₂ for 3 times, and then the mixture was stirred at 100° C. for 12 hours under N₂ atmosphere. LC-MS showed 5-bromo-2-tert-butyl-pyrimidine was consumed completely and one main peak with desired mass was detected. The reaction mixture was partitioned between water (20 mL) and EtOAc (30 mL). The organic phase was separated, washed with brine (30 mL), dried over Na₂SO₄, filtered and concentrated under reduced pressure to give a residue. The residue was purified by prep-TLC (Silica gel, petroleum ether/ethyl acetate=5/1) to give desired 5-benzylsulfanyl-2-tert-butyl-pyrimidine (580 mg, 2.24 mmol, 69.0% yield) as a white oil. MS (ESI): mass calcd. For C₁₅H₁₈N₂S 258.12, mass found 259.1 [M+H]⁺.

Step 2: To a solution of 5-benzylsulfanyl-2-tert-butyl-pyrimidine (100 mg, 387 μmol, 1 eq) in AcOH (1 mL) and H₂O (0.2 mL) was added NCS (155 mg, 1.16 mmol, 3 eq). The mixture was stirred at 20° C. for 1 hour. LC-MS showed 5-benzylsulfanyl-2-tert-butyl-pyrimidine was consumed completely and desired compound was detected. The reaction mixture was diluted with water (10 mL) and extracted with EtOAc (30 mL). The combined organic layers were washed with brine (10 mL), dried over anhydrous Na₂SO₄, filtered and concentrated under reduced pressure to give desired 2-tert-butylpyrimidine-5-sulfonyl chloride (130 mg, crude) as a colourless oil. MS (ESI): mass calcd. For C₈H₁₁ClN₂O₂S 234.02, mass found 284.0 [M+H+49]⁺.

Step 3: To a solution of 4-chloro-3-[7-(5-fluoropyrimidin-2-yl)-4, 7-diazaspiro [2.5]octan-4-yl]-1H-indazole (10 mg, 27.9 μmol, 1 eq) in DCM (2 mL), 2-tert-butylpyrimidine-5-sulfonyl chloride (13.1 mg, 55.7 μmol, 2 eq) was added TEA (8.46 mg, 83.6 μmol, 11.6 uL, 3 eq) and DMAP (341 ug, 2.79 μmol, 0.1 eq). The mixture was stirred at 25° C. for 12 hours. LC-MS showed 4-chloro-3-[7-(5-fluoropyrimidin-2-yl)-4, 7-diazaspiro [2.5]octan-4-yl]-1H-indazole was remained and desired mass was detected. The reaction was concentrated to get a residue. The residue was purified by prep-HPLC (column: Phenomenex luna C18 80*40 mm*3 m; mobile phase: [water(TFA)-ACN]; B %: 50%-100%, 8 min) to give desired 1-(2-tert-butylpyrimidin-5-yl) sulfonyl-4-chloro-3-[7-(5-fluoropyrimidin-2-yl)-4, 7-diazaspiro [2.5]octan-4-yl]indazole (5.6 mg, 10.0 μmol, 36.0% yield, 99.80% purity) as a white solid. ¹H NMR (DMSO-d₆) δ 9.11 (s, 2H), 8.46 (s, 2H), 8.08 (br d, 1H), 7.59-7.75 (m, 1H), 7.45-7.57 (m, 1H), 3.91 (br s, 2H), 3.55 (br s, 2H), 3.07 (s, 2H), 1.25 (s, 9H), 0.77 (br s, 2H), 0.40 (br s, 2H). HPLC: 99.80% (220 nm), 99.85% (210 nm), 100.00% (254 nm). MS (ESI): mass calcd. For C₂₅H₂₆ClFN₈O₂S 556.16, mass found 557.0 [M+H]⁺.

### Compound 101: 4-chloro-3-[7-(5-fluoropyrimidin-2-yl)-4, 7-diazaspiro [2.5]octan-4-yl]-1-[4-[1-(trifluoromethyl) cyclopropyl]phenyl]sulfonyl-indazole

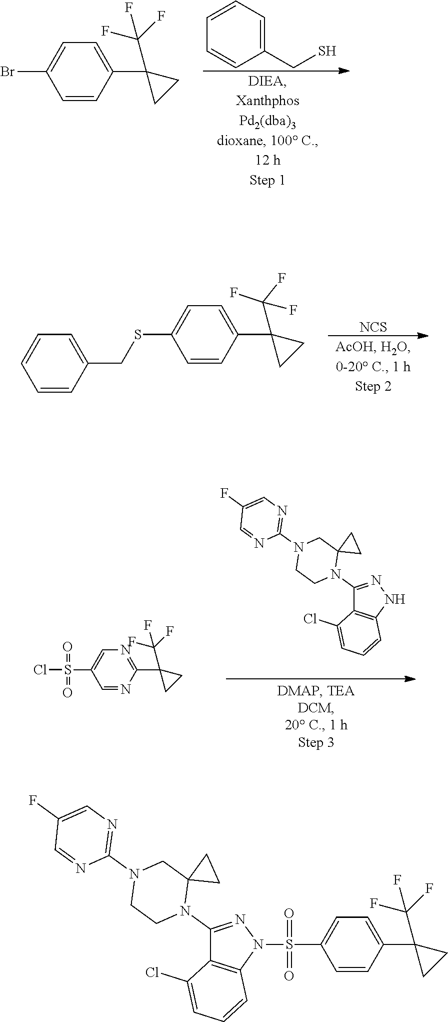

Step 1: A mixture of 1-bromo-4-[1-(trifluoromethyl)cyclopropyl]benzene (500 mg, 1.89 mmol, 1 eq), BnSH (281 mg, 2.26 mmol, 265 μL, 1.2 eq), Pd₂(dba)₃ (173 mg, 189 μmol, 0.1 eq), DIEA (731 mg, 5.66 mmol, 986 μL, 3 eq) and Xantphos (54.6 mg, 94.3 μmol, 0.05 eq) in dioxane(5 mL) was degassed and purged with N₂ for 3 times, and then the mixture was stirred at 100° C. for 12 hours under N₂ atmosphere. TLC indicated 1-bromo-4-[1-(trifluoromethyl) cyclopropyl]benzene was consumed completely and one new spot formed. The reaction mixture was filtered and partitioned between water (15 mL) and EtOAc (30 mL). The organic phase was separated, washed with brine (20 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜8% ethyl acetate/petroleum ether gradient @40 mL/min) to give desired 1-benzylsulfanyl-4-[1-(trifluoromethyl) cyclopropyl]benzene (450 mg, 1.46 mmol, 77.4% yield) as a white solid.

Step 2: To a solution of 1-benzylsulfanyl-4-[1-(trifluoromethyl) cyclopropyl]benzene (400 mg, 1.30 mmol, 1 eq) in AcOH (3 mL) H₂O (1 mL) was added NCS (693 mg, 5.19 mmol, 4 eq) at 0° C. The mixture was stirred at 20° C. for 1 hour. LC-MS showed 1-benzylsulfanyl-4-[1-(trifluoromethyl) cyclopropyl]benzene was consumed completely and desired mass was detected (the sample was quenched with piperidine). The reaction mixture was diluted with water (10 mL) and extracted with MTBE (20 mL). The combined organic layers were washed with brine (10 mL), dried over Na₂SO₄, filtered and concentrated under reduced pressure to give desired 4-[1-(trifluoromethyl) cyclopropyl]benzenesulfonyl chloride (350 mg, crude) as a yellow solid. MS (ESI): mass calcd. For C₁₀H₈SO₂ClF₃ 283.99, mass found 334.1 [M+49+H]⁺.

Step 3: To a solution of 4-chloro-3-[7-(5-fluoropyrimidin-2-yl)-4, 7-diazaspiro [2.5]octan-4-yl]-1H-indazole (50 mg, 139 μmol, 1 eq) in DCM (2 mL) was added DMAP (17.0 mg, 139 μmol, 1 eq), 4-[1-(trifluoromethyl)cyclopropyl]benzenesulfonyl chloride (198 mg, 697 μmol, 5 eq) and TEA (42.3 mg, 418 μmol, 58.2 μL, 3 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was partitioned between 5 mL of H₂O and 20 mL of EtOAc. The organic phase was separated, washed with 9 mL of brine, dried over Na₂SO₄, filtered and concentrated under reduced pressure to give the crude product. The residue was purified by prep-HPLC (TFA condition, column: Phenomenex luna C18 250*50 mm*10 μm; mobile phase: [water(TFA)-ACN]; B %: 60%-95%, 10 min) to give desired 4-chloro-3-[7-(5-fluoropyrimidin-2-yl)-4,7-diazaspiro[2.5]octan-4-yl]-1-[4-[1-(trifluoromethyl) cyclopropyl]phenyl]sulfonyl-indazole (3.5 mg, 5.69 μmol, 11.5% yield, 98.69% purity) as a gray solid. ¹H NMR (400 MHz, DMSO-d₆) δ 0.28-0.43 (m, 2H) 0.64 (s, 2H) 1.06-1.17 (m, 2H) 1.24-1.44 (m, 2H) 3.52 (br s, 4H) 3.92 (s, 2H) 7.44-7.50 (m, 1H) 7.58-7.69 (m, 3H) 7.80 (d, J=8.50 Hz, 2H) 8.08 (d, J=8.38 Hz, 1H) 8.47 (s, 2H). HPLC: 98.69% (220 nm), 98.65% (210 nm), 99.09% (254 nm). MS (ESI): mass calcd. For C₂₇H₂₃SO₂N₆ClF₄ 606.12, mass found 607.2 [M+H]⁺.

### Compound 102: 4-chloro-1-[4-(1, 1-difluoropropyl) phenyl]sulfonyl-3-[7-(5-fluoropyrimidin-2-yl)-4, 7-diazaspiro [2.5]octan-4-yl]indazole

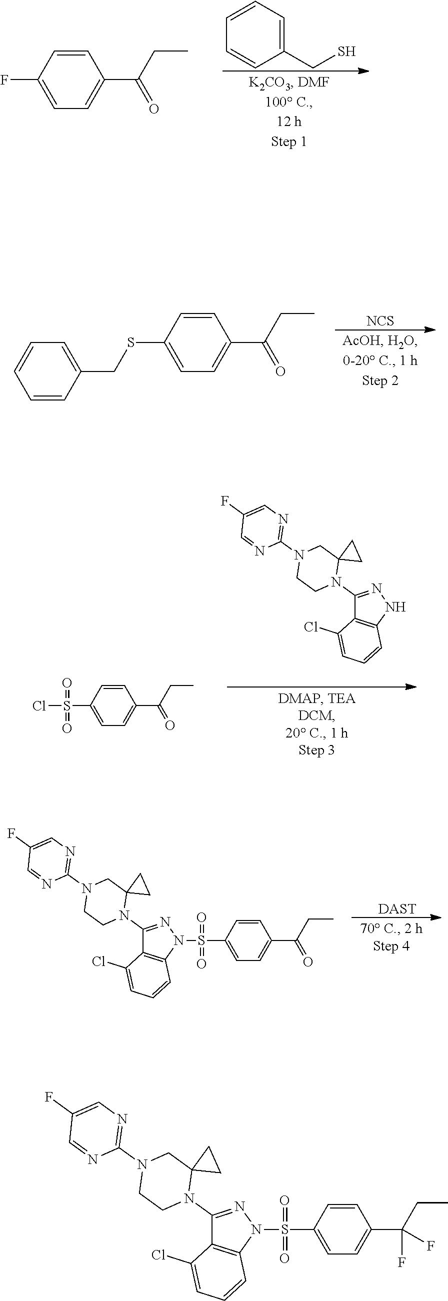

Step 1: A mixture of 1-(4-fluorophenyl) propan-1-one (3 g, 19.7 mmol, 2.74 mL, 1 eq), BnSH (2.94 g, 23.7 mmol, 2.78 mL, 1.2 eq) and K₂CO₃ (4.09 g, 29.6 mmol, 1.5 eq) in DMF (30 mL) was degassed and purged with N₂ for 3 times, and then the mixture was stirred at 100° C. for 12 hours under N₂ atmosphere. LC-MS showed 1-(4-fluorophenyl) propan-1-one was consumed completely and one main peak with desired mass was detected. The reaction mixture was partitioned between water (30 mL) and EtOAc (90 mL). The organic phase was separated, washed with brine (60 mL), dried over Na₂SO₄, filtered and concentrated under reduced pressure to give desired 1-(4-benzylsulfanylphenyl)propan-1-one (5 g, crude) as a brown oil. MS (ESI): mass calcd. For C₁₆H₁₆OS 256.09 mass found 257.1[M+H]⁺.

Step 2: To a solution of 1-(4-benzylsulfanylphenyl) propan-1-one (500 mg, 1.95 mmol, 1 eq) in AcOH (5 mL) and H₂O (1 mL) was added NCS (1.04 g, 7.80 mmol, 4 eq) at 0° C. The mixture was stirred at 20° C. for 1 hour. TLC indicated 1-(4-benzylsulfanylphenyl) propan-1-one was consumed completely and one major new spot with larger polarity was detected. The reaction mixture was concentrated under reduced pressure to give a residue. The residue was purified by prep-TLC (Silica gel, petroleum ether/ethyl acetate=5/1) to give desired 4-propanoylbenzenesulfonyl chloride (320 mg, 1.38 mmol, 70.5% yield) as a yellow gum.

Step 3: To a solution of 4-chloro-3-[7-(5-fluoropyrimidin-2-yl)-4,7-diazaspiro[2.5]octan-4-yl]-1H-indazole (200 mg, 557 μmol, 1 eq), 4-propanoylbenzenesulfonyl chloride (311 mg, 1.34 mmol, 2.4 eq) and TEA (169 mg, 1.67 mmol, 233 μL, 3 eq) in DCM (4 mL) was added DMAP (6.81 mg, 55.7 μmol, 0.1 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole remained and one main peak with desired mass was detected. The reaction mixture was concentrated under reduced pressure to give a residue. The residue was purified by column chromatography (Silica gel, petroleum ether/ethyl acetate=3/1) to give desired 1-[4-[4-chloro-3-[7-(5-fluoropyrimidin-2-yl)-4, 7-diazaspiro [2.5]octan-4-yl]indazol-1-yl]sulfonylphenyl]propan-1-one (300 mg, 541 μmol, 96.9% yield) as a yellow oil. MS (ESI): mass calcd. For C₂₆H₂₄N₆O₃SClF 554.13 mass found 555.1[M+H]⁺.

Step 4: A solvent of DAST (2 mL) in 1-[4-[4-chloro-3-[7-(5-fluoropyrimidin-2-yl)-4, 7-diazaspiro [2.5]octan-4-yl]indazol-1-yl]sulfonylphenyl]propan-1-one (100 mg, 180 μmol, 1 eq) was stirred at 70° C. for 2 hours. LC-MS showed 1-[4-[4-chloro-3-[7-(5-fluoropyrimidin-2-yl)-4, 7-diazaspiro [2.5]octan-4-yl]indazol-1-yl]sulfonylphenyl]propan-1-one remained and one main peak with desired mass was detected. The mixture was quenched by sat.aq.NaHCO₃ (20 mL) and extracted with DCM (30 mL), washed with 30 mL of brine, dried over Na₂SO₄, filtered and concentrated under reduced pressure to give a residue. The residue was purified by prep-TLC (Silica gel, petroleum ether/ethyl acetate=3/1) to give desired 4-chloro-1-[4-(1,1-difluoropropyl)phenyl]sulfonyl-3-[7-(5-fluoropyrimidin-2-yl)-4,7-diazaspiro[2.5]octan-4-yl]indazole (5.1 mg, 8.84 μmol, 4.91% yield, 100.00% purity) as a white solid. ¹H NMR (400 MHz, DMSO-d₆) δ 8.46 (d, J=0.6 Hz, 2H), 8.08 (d, J=8.0 Hz, 1H), 7.89 (d, J=8.4 Hz, 2H), 7.72-7.60 (m, 3H), 7.47 (d, J=7.4 Hz, 1H), 3.91 (s, 2H), 3.53 (br s, 4H), 2.22-1.96 (m, 2H), 0.80 (t, J=7.4 Hz, 3H), 0.78-0.71 (m, 2H), 0.36 (br s, 2H). HPLC: 100.00% (220 nm), 100.00% (215 nm), 100.00% (254 nm). MS (ESI): mass calcd. For C₂₆H₂₄N₆F₃SO₂Cl 576.13 mass found 577.1[M+H]⁺.

### Compound 103: 4-chloro-1-[4-(1, 1-difluoroethyl) phenyl]sulfonyl-7-fluoro-3-[7-(5-fluoropyrimidin-2-yl)-4, 7-diazaspiro [2.5]octan-4-yl]indazole

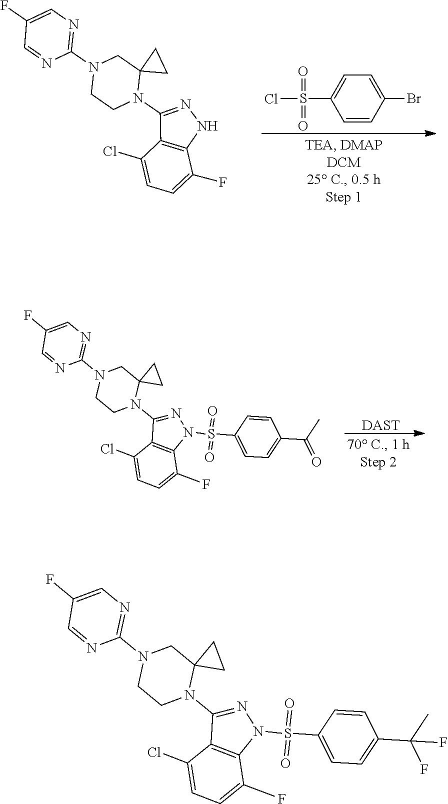

Step 1: To a solution of 4-chloro-7-fluoro-3-[7-(5-fluoropyrimidin-2-yl)-4,7-diazaspiro[2.5]octan-4-yl]-1H-indazole (110 mg, 292 μmol, 1 eq) and 4-acetylbenzenesulfonyl chloride (128 mg, 584 μmol, 2 eq) in DCM (2 mL) was added TEA (73.9 mg, 730 μmol, 2.5 eq) and DMAP (3.57 mg, 29.2 μmol, 0.1 eq). The mixture was stirred at 25° C. for 0.5 hour. LC-MS showed 4-chloro-7-fluoro-3-[7-(5-fluoropyrimidin-2-yl)-4,7-diazaspiro[2.5]octan-4-yl]-1H-indazole was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-TLC (Silica gel, petroleum ether/EtOAc=2/1) to give desired 1-[4-[4-chloro-7-fluoro-3-[7-(5-fluoropyrimidin-2-yl)-4, 7-diazaspiro [2.5]octan-4-yl]indazol-1-yl]sulfonylphenyl]ethanone (130 mg, 233 μmol, 79.7% yield) as a yellow oil. MS (ESI): mass calcd. For C₂₅H₂₁N₆O₃SClF₂ 558.11, mass found 559.1[M+H]⁺.

Step 2: A mixture of 1-[4-[4-chloro-7-fluoro-3-[7-(5-fluoropyrimidin-2-yl)-4,7-diazaspiro[2.5]octan-4-yl]indazol-1-yl]sulfonylphenyl]ethanone (100 mg, 179 μmol, 1 eq) in DAST (1.22 g, 7.57 mmol, 1 mL, 42.3 eq) was degassed and purged with N₂ for 3 times, and then the mixture was stirred at 70° C. for 1 hour under N₂ atmosphere. LC-MS showed 1-[4-[4-chloro-7-fluoro-3-[7-(5-fluoropyrimidin-2-yl)-4, 7-diazaspiro [2.5]octan-4-yl]indazol-1-yl]sulfonylphenyl]ethanone was consumed completely and desired mass was detected. The crude was added H₂O (5 mL), and extracted with EtOAc (5 mL*3). The combined organic layers were washed with brine (5 mL), dried over Na₂SO₄, filtered and concentrated under reduced pressure to give a residue. The residue was purified by prep-HPLC (neutral condition, column: Waters Xbridge BEH C18 100*25 mm*5 μm; mobile phase: [water(10 mM NH₄HCO₃)-ACN]; B %: 40%-70%, 10 min) to give desired 4-chloro-1-[4-(1, 1-difluoroethyl) phenyl]sulfonyl-7-fluoro-3-[7-(5-fluoropyrimidin-2-yl)-4, 7-diazaspiro [2.5]octan-4-yl]indazole (27 mg, 45.7 μmol, 29.5% yield, 98.33% purity) as a yellow solid. ¹H NMR (400 MHz, DMSO-d₆) δ 8.46 (s, 2H), 7.87 (d, J=8.4 Hz, 2H), 7.73 (d, J=8.5 Hz, 2H), 7.56 (dd, J=8.8, 10.9 Hz, 1H), 7.49-7.44 (m, 1H), 3.88 (s, 2H), 3.52-3.50 (m, 4H), 1.91 (t, J=19.1 Hz, 3H), 0.74 (br s, 2H), 0.38 (br s, 2H). HPLC: 98.33% (220 nm), 98.28% (215 nm), 98.83% (254 nm). MS (ESI): mass calcd. For C₂₅H₂₁N₆ClSO₂F₄ 580.11, mass found 581.1[M+H]⁺.

### Compound 104:4-chloro-1-[4-(1, 1-difluoroethyl) phenyl]sulfonyl-3-[2-(trifluoromethyl) azetidin-1-yl]indazole

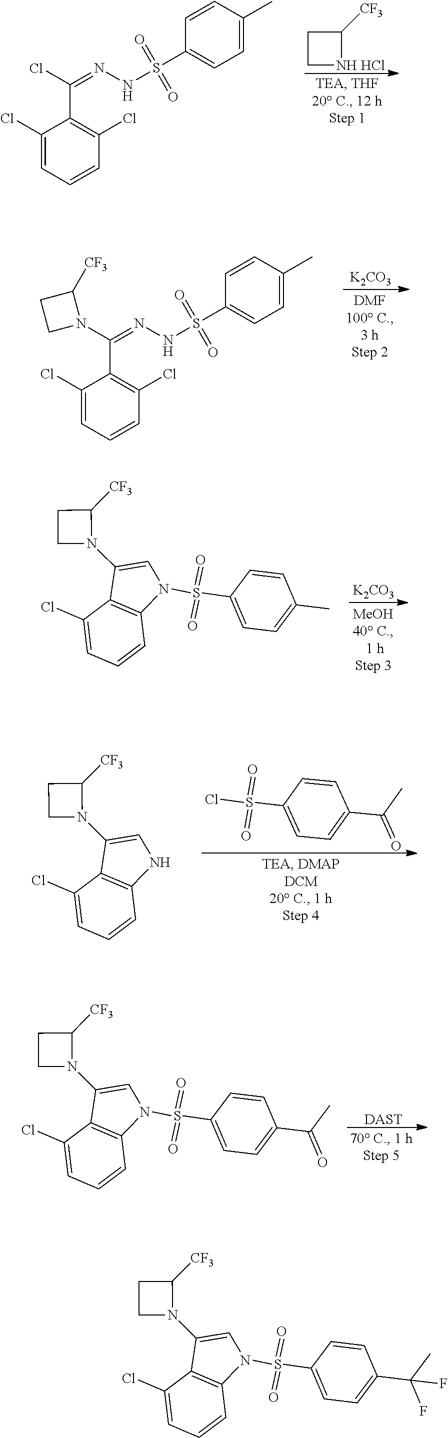

Step 1: To a solution of (1E)-2, 6-dichloro-N-(p-tolylsulfonyl) benzohydrazonoyl chloride (701 mg, 1.86 mmol, 1.5 eq) and 2-(trifluoromethyl) azetidine; hydrochloride (200 mg, 1.24 mmol, 1 eq) in THF (10 mL) was added TEA (1.25 g, 12.4 mmol, 1.72 mL, 10 eq). The mixture was stirred at 20° C. for 12 hours. LC-MS showed (1E)-2, 6-dichloro-N-(p-tolylsulfonyl) benzohydrazonoyl chloride was consumed completely and desired mass was detected. The reaction mixture was partitioned between water (30 mL) and EtOAc (90 mL). The organic phase was separated, washed with brine (100 mL) dried over Na₂SO₄, filtered and concentrated under reduced pressure to give desired N-[(E)-[(2, 6-dichlorophenyl)-[2-(trifluoromethyl) azetidin-1-yl]methylene]amino]-4-methyl-benzenesulfonamide (860 mg, crude) as a yellow solid. MS (ESI): mass calcd. For C₁₈H₁₆N₃SO₂Cl₂F₃ 465.03, mass found 466.0 [M+H]⁺.

Step 2: To a solution of N-[(E)-[(2, 6-dichlorophenyl)-[2-(rifluoromethyl) azetidin-1-yl]methylene]amino]-4-methyl-benzenesulfonamide (860 mg, 1.84 mmol, 1 eq) in DMF (10 mL) as added K₂CO₃ (1.02 g, 7.38 mmol, 4 eq). The mixture was stirred at 100° C. for 3 hours. LC-MS showed N-[(E)-[(2, 6-dichlorophenyl)-[2-(rifluoromethyl) azetidin-1-yl]methylene]amino]-4-methyl-benzenesulfonamide was consumed completely and desired mass was detected. The reaction mixture was partitioned between water (30 mL) and EtOAc (90 mL). The organic phase was separated, washed with brine (100 ml), dried over Na₂SO₄, filtered and concentrated under reduced pressure to give desired 4-chloro-1-(p-tolylsulfonyl)-3-[2-(trifluoromethyl) azetidin-1-yl]indazole (660 mg, crude) as a yellow solid. MS (ESI): mass calcd. For C₁₈H₁₅N₃ClSO₂F₃ 429.05, mass found 430.0 [M+H]⁺.

Step 3: To a solution of 4-chloro-1-(p-tolylsulfonyl)-3-[2-(trifluoromethyl) azetidin-1-yl]indazole (660 mg, 1.54 mmol, 1 eq) in MeOH (10 mL) was added K₂CO₃ (1.06 g, 7.68 mmol, 5 eq). The mixture was stirred at 40° C. for 1 hour. LC-MS showed 4-chloro-1-(p-tolylsulfonyl)-3-[2-(trifluoromethyl) azetidin-1-yl]indazole was consumed completely and desired mass was detected. The reaction mixture was partitioned between water (20 mL) and EtOAc (60 mL). The organic phase was separated, washed with brine (100 mL), dried over Na₂SO₄, filtered and concentrated under reduced pressure to give desired 4-chloro-3-[2-(trifluoromethyl) azetidin-1-yl]-1H-indazole (423 mg, crude) as a yellow solid. MS (ESI): mass calcd. For C₁₁H₉N₃ClF₃ 275.04, mass found 276.1 [M+H]⁺.

Step 4: To a solution of 4-chloro-3-[2-(trifluoromethyl) azetidin-1-yl]-1H-indazole (50.0 mg, 181 μmol, 1 eq) and 4-acetylbenzenesulfonyl chloride (317 mg, 1.45 mmol, 8 eq) in DCM (4 mL) was added DMAP (22.1 mg, 181 μmol, 1 eq) and TEA (2.18 g, 21.5 mmol, 3 mL, 119 eq). The mixture was stirred at 20° C. for 1 hour. LC-MS showed 4-chloro-3-[2-(trifluoromethyl) azetidin-1-yl]-1H-indazole was consumed completely and desired mass was detected. The reaction mixture was filtered and the filtrate was concentrated under reduced pressure to give a crude. The residue was purified by prep-TLC (Silica gel, petroleum ether/EtOAc=2/1) to give desired 1-[4-[4-chloro-3-[2-(trifluoromethyl) azetidin-1-yl]indazol-1-yl]sulfonylphenyl]ethanone (61.0 g, 133 μmol, 73.5% yield) as an orange oil. MS (ESI): mass calcd. For C₁₉H₁₅N₃ClSO₃F₃ 457.05, mass found 458.1 [M+H]⁺.

Step 5: A mixture of 1-[4-[4-chloro-3-[2-(trifluoromethyl) azetidin-1-yl]indazol-1-yl]sulfonylphenyl]ethanone (50 mg, 109 μmol, 1 eq) in DAST (1.22 g, 7.57 mmol, 1 mL, 69.3 eq) was degassed and purged with N₂ for 3 times, and then the mixture was stirred at 70° C. for 1 hour under N₂ atmosphere. LC-MS showed 1-[4-[4-chloro-3-[2-(trifluoromethyl) azetidin-1-yl]indazol-1-yl]sulfonylphenyl]ethanone was consumed completely and desired mass was detected. The crude was added H₂O (5 mL), and extracted with EtOAc (5 mL*3). The combined organic layers were washed with brine (5 mL), dried over Na₂SO₄, filtered and concentrated under reduced pressure to give a residue. The residue was purified by prep-HPLC (TFA condition, column: Phenomenex luna C18 250*50 mm*10 m; mobile phase: [water(TFA)-ACN]; B %: 60%-95%, 10 min) to give desired 4-chloro-1-[4-(1,1-difluoroethyl) phenyl]sulfonyl-3-[2-(trifluoromethyl) azetidin-1-yl]indazole (10.3 mg, 21.1 μmol, 19.3% yield, 98.22% purity) as a white solid. ¹H NMR (400 MHz, DMSO-d₆) δ 1.79-1.98 (m, 3H) 2.35-2.45 (m, 1H) 2.50-2.65 (m, 1H) 3.80-4.01 (m, 1H) 4.27-4.52 (m, 1H) 5.08-5.40 (m, 1H) 7.43 (d, J=7.89 Hz, 1H) 7.56-7.66 (m, 1H) 7.70 (d, J=8.55 Hz, 2H) 7.87 (d, J=8.33 Hz, 2H) 8.00-8.08 (m, 1H). HPLC: 98.22% (220 nm), 98.22% (215 nm), 93.08 (254 nm). MS (ESI): mass calcd. For C₁₉H₁₅ClN₃SO₂F₅ 479.05 mass found 480.1 [M+H]⁺.

### Compound 105: 4-chloro-1-((4-(1, 1-difluoroethyl) phenyl) sulfonyl)-3-(2-(2-fluoropropan-2-yl)-4-(5-fluoropyrimidin-2-yl) piperazin-1-yl)-1H-indazole

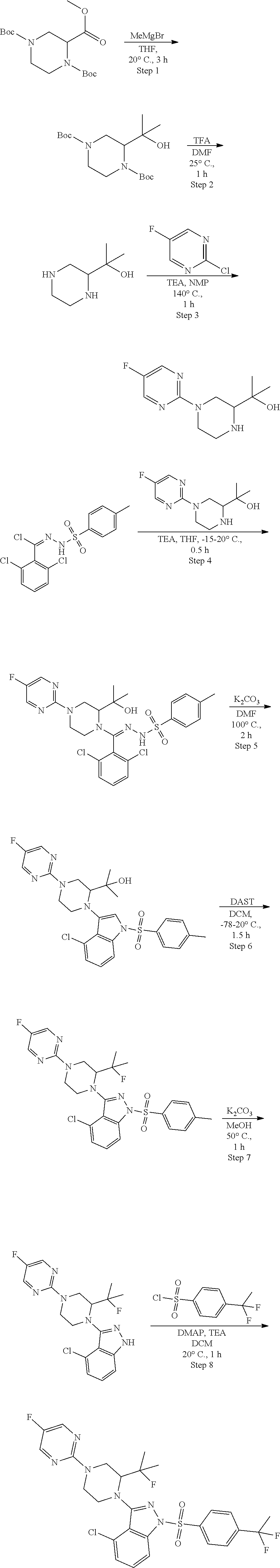

Step 1: To a solution of O1, O4-ditert-butyl 02-methyl piperazine-1, 2, 4-tricarboxylate (500 mg, 1.45 mmol, 1.0 eq) in THF (5 mL) was added MeMgBr (3 M, 1.45 mL, 3 eq) at 0° C. and the solution was stirred at 20° C. for 3 hours. LCMS showed O1, O4-ditert-butyl 02-methyl piperazine-1, 2, 4-tricarboxylate was consumed and desired mass was detected. The reaction was added saturated NH₄Cl solution (5 mL) and extracted with MTBE (2*10 mL). The combined organics were concentrated to get a residue. The residue was purified by flash silica gel chromatography (ISCO®; 40 g SepaFlash® Silica Flash Column, Eluent of 0˜50% ethyl acetate/petroleum ether gradient @80 mL/min) to give desired ditert-butyl 2-(1-hydroxy-1-methyl-ethyl)piperazine-1,4-dicarboxylate (390 mg, 1.13 mmol, 78.0% yield) as a colorless oil. ¹H NMR (DMSO-d₆) δ 3.71-4.29 (m, 4H), 3.10-3.47 (m, 2H), 2.96-3.09 (m, 1H), 1.47 (d, 18H), 1.31 (br s, 3H), 1.22 (s, 3H). MS (ESI): mass calcd. For C₁₇H₃₂N₂O₅ 344.23 mass found 345.4 [M+H]⁺.

Step 2: The solution of ditert-butyl 2-(1-hydroxy-1-methyl-ethyl) piperazine-1, 4-dicarboxylate (390 mg, 1.13 mmol, 1 eq) in TFA (4 mL) and DCM (20 mL) was stirred at 25° C. for 1 hour. TLC showed ditert-butyl 2-(1-hydroxy-1-methyl-ethyl) piperazine-1, 4-dicarboxylate was consumed completely and a new spot was detected. The reaction was concentrated to give desired 2-piperazin-2-ylpropan-2-ol (290 mg, crude, TFA) as a yellow oil.

Step 3: The solution of 2-piperazin-2-ylpropan-2-ol (290 mg, 1.12 mmol, 1 eq, TFA) and 2-chloro-5-fluoro-pyrimidine (148 mg, 1.12 mmol, 1 eq) in TEA (2.27 g, 22.4 mmol, 20 eq) and NMP (3 mL) was stirred at 140° C. for 1 hour. LCMS showed 2-piperazin-2-ylpropan-2-ol was consumed completely and desired mass was detected. The reaction was concentrated to give desired 2-[4-(5-fluoropyrimidin-2-yl) piperazin-2-yl]propan-2-ol (269 mg, crude) as a yellow oil. MS (ESI): mass calcd. For C₁₁H₁₇N₄OF 240.14 mass found 241.1 [M+H]⁺.

Step 4: To the solution of 2-[4-(5-fluoropyrimidin-2-yl)piperazin-2-yl]propan-2-ol (269 mg, 1.12 mmol, 1 eq) and TEA (1.13 g, 11.2 mmol, 10 eq) in THF (6 mL) was added (1E)-2,6-dichloro-N-(p-tolylsulfonyl)benzohydrazonoyl chloride (630 mg, 1.67 mmol, 1.49 eq) at −15° C. and the solution was stirred at 20° C. for 0.5 hour. LCMS showed 2-[4-(5-fluoropyrimidin-2-yl) piperazin-2-yl]propan-2-ol remained and desired mass was detected. The reaction was concentrated to give desired N-[(E)-[(2, 6-dichlorophenyl)-[4-(5-fluoropyrimidin-2-yl)-2-(1-hydroxy-1-methyl-ethyl) piperazin-1-yl]methylene]amino]-4-methyl-benzenesulfonamide (650 mg, crude) as a yellow oil. MS (ESI): mass calcd. For C₂₅H₂₇N₆SO₃Cl₂F 580.12 mass found 581.1 [M+H]⁺.

Step 5: The solution of N-[(E)-[(2, 6-dichlorophenyl)-[4-(5-fluoropyrimidin-2-yl)-2-(1-hydroxy-1-methyl-ethyl) piperazin-1-yl]methylene]amino]-4-methyl-benzenesulfonamide (650 mg, 1.12 mmol, 1 eq) and K₂CO₃ (2.32 g, 16.8 mmol, 15 eq) in DMF (5 mL) was stirred at 100° C. for 2 hours. LCMS showed N-[(E)-[(2, 6-dichlorophenyl)-[4-(5-fluoropyrimidin-2-yl)-2-(1-hydroxy-1-methyl-ethyl) piperazin-1-yl]methylene]amino]-4-methyl-benzenesulfonamide was consumed completely and desired mass was detected. The reaction was added saturated NH₄Cl solution (5 mL) and extracted with MTBE (2*10 mL). The combined organics were concentrated to get a residue. The residue was purified by prep-TLC (petroleum ether/ethyl acetate=2/1) to give desired 2-[1-[4-chloro-1-(p-tolylsulfonyl) indazol-3-yl]-4-(5-fluoropyrimidin-2-yl) piperazin-2-yl]propan-2-ol (100 mg, crude) as a yellow solid. MS (ESI): mass calcd. For C₂₅H₂₆N₆SO₃ClF 544.15 mass found 545.2 [M+H]⁺.

Step 6: To the solution of 2-[1-[4-chloro-1-(p-tolylsulfonyl) indazol-3-yl]-4-(5-fluoropyrimidin-2-yl)piperazin-2-yl]propan-2-ol (90 mg, 165 μmol, 1 eq) in DCM (3 mL) was added DAST (266 mg, 1.65 mmol, 10 eq) at −78° C. and the solution was stirred at −78° C. for 0.5 hour. Then the solution was stirred at 20° C. for 1 hour. TLC showed 2-[1-[4-chloro-1-(p-tolylsulfonyl) indazol-3-yl]-4-(5-fluoropyrimidin-2-yl) piperazin-2-yl]propan-2-ol was consumed completely and a new spot with lower priority. The reaction was quenched with MeOH (0.2 mL) and concentrated to give desired 4-chloro-3-[2-(1-fluoro-1-methyl-ethyl)-4-(5-fluoropyrimidin-2-yl) piperazin-1-yl]-1-(p-tolylsulfonyl) indazole (90 mg, crude) as a yellow solid.

Step 7: The reaction mixture of 4-chloro-3-[2-(1-fluoro-1-methyl-ethyl)-4-(5-fluoropyrimidin-2-yl) piperazin-1-yl]-1-(p-tolylsulfonyl) indazole (50 mg, 91.4 μmol, 1 eq) and K₂CO₃ (63.2 mg, 457 μmol, 5 eq) in MeOH (3 mL) was stirred at 50° C. for 1 hour. LCMS showed 4-chloro-3-[2-(1-fluoro-1-methyl-ethyl)-4-(5-fluoropyrimidin-2-yl) piperazin-1-yl]-1-(p-tolylsulfonyl) indazole was consumed completely and desired mass was detected. The reaction was concentrated to get a residue. The residue was purified by prep-TLC (petroleum ether/ethyl acetate=2/1) to give desired 4-chloro-3-[2-(1-fluoro-1-methyl-ethyl)-4-(5-fluoropyrimidin-2-yl) piperazin-1-yl]-1H-indazole (25.8 mg, 65.7 μmol, 71.9% yield) as a yellow solid. MS (ESI): mass calcd. For C₁₈H₁₉N₆ClF₂ 392.13 mass found 393.0 [M+H]⁺.

Step 8: To a solution of 4-chloro-3-[2-(1-fluoro-1-methyl-ethyl)-4-(5-fluoropyrimidin-2-yl)piperazin-1-yl]-1H-indazole (10 mg, 25.5 μmol, 1 eq) in DCM (1 mL) was added 4-(1,1-difluoroethyl)benzenesulfonyl chloride (9.19 mg, 38.2 μmol, 1.5 eq), DMAP (3.11 mg, 25.5 μmol, 1 eq) and TEA (12.9 mg, 127 μmol, 17.7 μL, 5 eq). The mixture was stirred at 20° C. for 1 hour. LCMS showed 4-chloro-3-[2-(1-fluoro-1-methyl-ethyl)-4-(5-fluoropyrimidin-2-yl) piperazin-1-yl]-1H-indazole remained and desired mass was detected. The reaction was concentrated to get a residue. The residue was purified by purified by prep-HPLC (TFA condition; column: Phenomenex Luna C18 75*30 mm*3 μm; mobile phase: [water(TFA)-ACN]; B %: 60%-90%, 8 min) to give desired 4-chloro-1-[4-(1,1-difluoroethyl)phenyl]sulfonyl-3-[2-(1-fluoro-1-methyl-ethyl)-4-(5-fluoropyrimidin-2-yl)piperazin-1-yl]indazole (1 mg, 1.67 μmol, 6.67% yield, 100.00% purity) as a white solid. ¹H NMR (DMSO-d₆) δ 8.49 (s, 2H), 8.09 (br d, J=8.5 Hz, 1H), 7.87 (br d, J=8.1 Hz, 2H), 7.76-7.60 (m, 3H), 7.51 (br d, J=7.6 Hz, 1H), 4.32-4.14 (m, 2H), 3.92-3.58 (m, 3H), 3.51 (br d, J=12.9 Hz, 1H), 3.20-3.07 (m, 1H), 1.89 (br t, J=19.1 Hz, 3H), 1.32-1.10 (m, 6H) HPLC: 100.00% (220 nm), 100.00% (215 nm), 100.00% (254 nm). MS (ESI): mass calcd. For C₂₆H₂₅N₆F₄SO₂Cl 596.14 mass found 597.1 [M+H]⁺.

### Compound 106: 4-chloro-1-[4-(1,1-difluoroethyl)phenyl]sulfonyl-3-[(2S)-4-(5-fluoropyrimidin-2-yl)-2-(trifluoromethyl)piperazin-1-yl]indazole

### Compound 107: 4-chloro-1-[4-(1, 1-difluoroethyl) phenyl]sulfonyl-3-[(2R)-4-(5-fluoropyrimidin-2-yl)-2-(trifluoromethyl) piperazin-1-yl]indazole

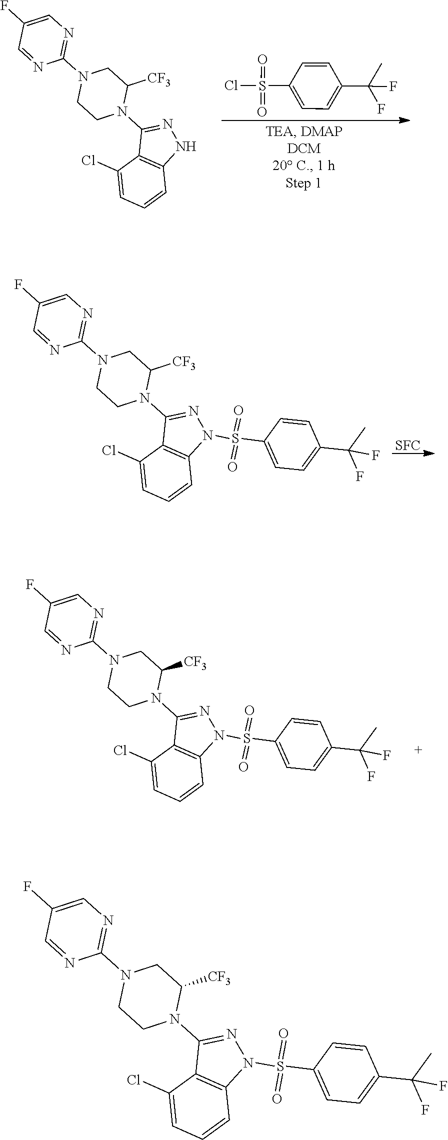

Step 1: To a solution of 4-chloro-3-[4-(5-fluoropyrimidin-2-yl)-2-(trifluoromethyl)piperazin-1-yl]-1H-indazole (40 mg, 99.8 μmol, 1 eq) and 4-(1,1-difluoroethyl)benzenesulfonyl chloride (36.0 mg, 150 μmol, 1.5 eq) in DCM (1 mL) was added TEA (20.2 mg, 200 μmol, 2 eq) and DMAP (1.22 mg, 9.98 μmol, 0.1 eq). The mixture was stirred at 20° C. for 1 hour. LC-MS showed -chloro-3-[4-(5-fluoropyrimidin-2-yl)-2-(trifluoromethyl) piperazin-1-yl]-1H-indazole was consumed completely and desired mass was detected. The reaction mixture was partitioned between H₂O (10 mL) and EtOAc (10 mL). The organic phase was separated, washed with brine (50 mL), dried over Na₂SO₄, filtered and concentrated under reduced pressure to give the crude product. The residue was purified by SFC (column: REGIS(S,S)WHELK-O1(250 mm*25 mm, 10 μm); mobile phase: [CO₂-i-PrOH (0.1% NH₃·H₂O)]; B %: 38%-38%, 10 min) to give two isomers. The structures were assigned randomly. 4-Chloro-1-[4-(1,1-difluoroethyl)phenyl]sulfonyl-3-[(2S)-4-(5-fluoropyrimidin-2-yl)-2-(trifluoromethyl)piperazin-1-yl]indazole (11.6 mg, 19.2 μmol, 19.2% yield, 98.47% purity, Rt=1.223 minutes; E.E. by chiral HPLC (%)=99.56%) was isolated as a white solid. ¹H NMR (400 MHz, DMSO-d₆) δ 8.58 (s, 2H), 8.18 (d, J=8.4 Hz, 1H), 8.02 (d, J=8.4 Hz, 2H), 7.81-7.73 (m, 3H), 7.59 (d, J=7.5 Hz, 1H), 4.93 (br d, J=14.5 Hz, 1H), 4.86-4.78 (m, 1H), 4.48 (br d, J=12.8 Hz, 1H), 3.87 (br dd, J=3.6, 14.4 Hz, 1H), 3.79-3.70 (m, 1H), 3.57 (br d, J=13.4 Hz, 1H), 3.26-3.18 (m, 1H), 1.96 (t, J=19.0 Hz, 3H). HPLC: 98.47% (220 nm), 98.06% (215 nm), 99.70% (254 nm). MS (ESI): mass calcd. For C₂₄H₁₉N₆F₆SO₂Cl 604.09, mass found 605.1[M+H]⁺. 4-chloro-1-[4-(1, 1-difluoroethyl) phenyl]sulfonyl-3-[(2R)-4-(5-fluoropyrimidin-2-yl)-2-(trifluoromethyl) piperazin-1-yl]indazole (4 mg, 6.61 μmol, 6.62% yield, 99.85% purity, Rt=1.353 minutes; E.E. by chiral HPLC (%)=99.16%) was isolated as a white solid. ¹H NMR (400 MHz, DMSO-d₆) δ 8.52 (s, 2H), 8.12 (d, J=8.5 Hz, 1H), 7.95 (d, J=8.4 Hz, 2H), 7.74-7.66 (m, 3H), 7.53 (d, J=7.6 Hz, 1H), 4.87 (br d, J=14.3 Hz, 1H), 4.81-4.70 (m, 1H), 4.41 (br d, J=12.8 Hz, 1H), 3.81 (br dd, J=3.4, 14.3 Hz, 1H), 3.67 (br d, J=10.6 Hz, 1H), 3.51 (br d, J=13.3 Hz, 1H), 3.18-3.11 (m, 1H), 1.89 (t, J=19.1 Hz, 3H). HPLC: 99.85% (220 nm), 98.36% (215 nm), 99.73% (254 nm). MS (ESI): mass calcd. For C₂₄H₁₉N₆F₆SO₂Cl 604.09, mass found 605.1[M+H]⁺.

### Compound 108: 3-[2-tert-butyl-4-(5-fluoropyrimidin-2-yl) piperazin-1-yl]-4-chloro-1-[4-(1, 1-difluoroethyl) phenyl]sulfonyl-indazole

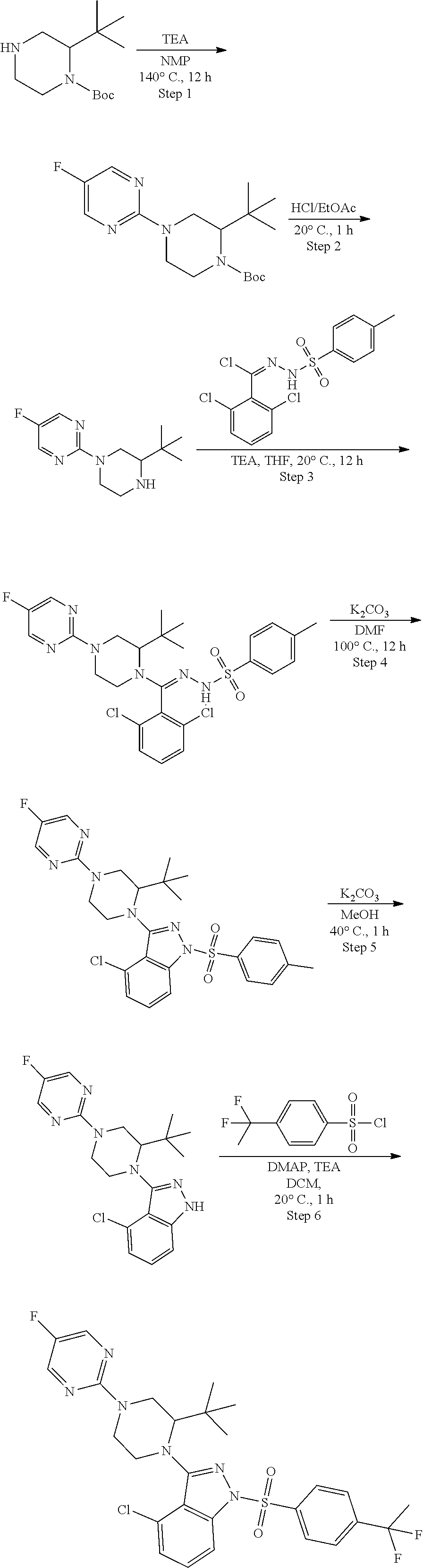

Step 1: To a solution of tert-butyl 2-tert-butylpiperazine-1-carboxylate (300 mg, 1.24 mmol, 1 eq) and 2-chloro-5-fluoro-pyrimidine (213 mg, 1.61 mmol, 198 μL, 1.3 eq) in NMP (3 mL) was added TEA (125 mg, 1.24 mmol, 172 μL, 1 eq). The mixture was stirred at 140° C. for 12 hours. LC-MS showed tert-butyl 2-tert-butylpiperazine-1-carboxylate was consumed completely and desired mass was detected. The reaction mixture was partitioned between H₂O (30 mL) and MTBE (50 mL). The organic phase was separated, washed with brine (50 mL), dried over Na₂SO₄, filtered and concentrated under reduced pressure to give the crude product. The residue was purified by flash silica gel chromatography (ISCO®; 2 g SepaFlash® Silica Flash Column, Eluent of 0˜5% ethyl acetate/petroleum ether gradient @60 mL/min) to give desired tert-butyl 2-tert-butyl-4-(5-fluoropyrimidin-2-yl)piperazine-1-carboxylate (410 mg, 1.21 mmol, 97.9% yield) as a white solid. MS (ESI): mass calcd. For C₁₇H₂₇N₄FO₂ 338.21, mass found 339.2 [M+H]⁺.

Step 2: To a solution of tert-butyl 2-tert-butyl-4-(5-fluoropyrimidin-2-yl) piperazine-1-carboxylate (410 mg, 1.21 mmol, 1 eq) in HCl/EtOAc (4M, 5 mL). The mixture was stirred at 20° C. for 1 hour. LC-MS showed tert-butyl 2-tert-butyl-4-(5-fluoropyrimidin-2-yl) piperazine-1-carboxylate was consumed completely and desired mass was detected. The reaction mixture was partitioned between H₂O (10 mL) and EtOAc (10 mL). The organic phase was separated, washed with brine (50 mL), dried over Na₂SO₄, filtered and concentrated under reduced pressure to give desired 2-(3-tert-butylpiperazin-1-yl)-5-fluoro-pyrimidine (330 mg, crude, HCl) as a white solid. MS (ESI): mass calcd. For C₁₂H₂₀N₄FCl 238.16, mass found 239.1 [M+H]⁺.

Step 3: To a solution of 2-(3-tert-butylpiperazin-1-yl)-5-fluoro-pyrimidine (330 mg, 1.20 mmol, 1 eq, HCl) and (1E)-2,6-dichloro-N-(p-tolylsulfonyl) benzohydrazonoyl chloride (680 mg, 1.80 mmol, 1.5 eq) in THF (10 mL) was added TEA (608 mg, 6.01 mmol, 836 μL, 5 eq). The mixture was stirred at 20° C. for 12 hours. LC-MS showed 2-(3-tert-butylpiperazin-1-yl)-5-fluoro-pyrimidine was consumed completely and desired mass was detected. The reaction mixture was partitioned between H₂O (40 mL) and EtOAc (50 mL). The organic phase was separated, washed with brine (50 mL), dried over Na₂SO₄, filtered and concentrated under reduced pressure to give desired N-[(E)-[[2-tert-butyl-4-(5-fluoropyrimidin-2-yl) piperazin-1-yl]-(2,6-dichlorophenyl)methylene]amino]-4-methyl-benzenesulfonamide (690 mg, crude) as a white solid. MS (ESI): mass calcd. For C₂₆H₂₉N₆SO₂Cl₂F 578.14, mass found 579.1 [M+H]⁺.

Step 4: To a solution of N-[(E)-[[2-tert-butyl-4-(5-fluoropyrimidin-2-yl) piperazin-1-yl]-(2, 6-dichlorophenyl) methylene]amino]-4-methyl-benzenesulfonamide (0.69 g, 1.19 mmol, 1 eq) in DMF (8 mL) was added K₂CO₃ (823 mg, 5.95 mmol, 5 eq). The mixture was stirred at 100° C. for 12 hours. LC-MS showed N-[(E)-[[2-tert-butyl-4-(5-fluoropyrimidin-2-yl) piperazin-1-yl]-(2, 6-dichlorophenyl) methylene]amino]-4-methyl-benzenesulfonamide was consumed completely and desired mass was detected. The reaction mixture was partitioned between H₂O (20 mL) and MTBE (50 mL). The organic phase was separated, washed with brine (40 mL), dried over Na₂SO₄, filtered and concentrated under reduced pressure to give desired 3-[2-tert-butyl-4-(5-fluoropyrimidin-2-yl) piperazin-1-yl]-4-chloro-1-(p-tolylsulfonyl) indazole (640 mg, crude) as a white solid. MS (ESI): mass calcd. For C₂₆H₂₈N₆SO₂ClF 542.17, mass found 543.2 [M+H]⁺.

Step 5: To a solution of 3-[2-tert-butyl-4-(5-fluoropyrimidin-2-yl) piperazin-1-yl]-4-chloro-1-(p-tolylsulfonyl) indazole (640 mg, 1.18 mmol, 1 eq) in MeOH (7 mL) was added K₂CO₃ (814 mg, 5.89 mmol, 5 eq). The mixture was stirred at 40° C. for 1 hour. LC-MS showed 3-[2-tert-butyl-4-(5-fluoropyrimidin-2-yl) piperazin-1-yl]-4-chloro-1-(p-tolylsulfonyl) indazole was consumed completely and desired mass was detected. The reaction mixture was filtered and the filter liquor was concentrated under reduced pressure to give a residue. The residue was purified by prep-TLC (Silica gel, petroleum ether/ethyl acetate=2/1) to give desired 3-[2-tert-butyl-4-(5-fluoropyrimidin-2-yl)piperazin-1-yl]-4-chloro-1H-indazole (220 mg, 566 μmol, 48.00% yield) as a yellow oil. MS (ESI): mass calcd. For C₁₉H₂₂N₆ClF 388.16, mass found 389.1 [M+H]⁺.

Step 6: To a solution of 3-[2-tert-butyl-4-(5-fluoropyrimidin-2-yl) piperazin-1-yl]-4-chloro-1H-indazole (30 mg, 77.2 μmol, 1 eq) and 4-(1,1-difluoroethyl) benzenesulfonyl chloride (27.9 mg, 116 μmol, 1.5 eq) in DCM (2 mL) was added TEA (15.6 mg, 154 μmol, 21.5 μL, 2 eq) and DMAP (942 pg, 7.71 μmol, 0.1 eq). The mixture was stirred at 20° C. for 1 hour. LC-MS showed 3-[2-tert-butyl-4-(5-fluoropyrimidin-2-yl) piperazin-1-yl]-4-chloro-1H-indazole was consumed completely and desired mass was detected. The reaction mixture was partitioned between H₂O (10 mL) and DCM (10 mL). The organic phase was separated, washed with brine (50 mL), dried over Na₂SO₄, filtered and concentrated under reduced pressure to give the crude product. The residue was purified by prep-HPLC (TFA condition; Method:column: Phenomenex Luna C18 75*30 mm*3 pm; mobile phase: [water(TFA)-ACN]; B %: 55%-85%, 8 min) to give desired 3-[2-tert-butyl-4-(5-fluoropyrimidin-2-yl) piperazin-1-yl]-4-chloro-1-[4-(1,1-difluoroethyl) phenyl]sulfonyl-indazole (10 mg, 16.6 μmol, 21.52% yield, 98.46% purity) as a white solid. ¹H NMR (DMSO-d₆) δ 8.49 (s, 2H), 8.10 (d, 1H), 7.82 (d, 2H), 7.59-7.73 (m, 3H), 7.51 (d, 1H), 4.53 (m, 1H), 4.14 (m, 1H), 3.68-3.82 (m, 1H), 3.52-3.67 (m, 2H), 3.37-3.47 (m, 2H), 1.89 (m, 3H), 0.64 (s, 9H). HPLC: 98.46% (220 nm), 95.04% (215 nm), 98.21 (254 nm). MS (ESI): mass calcd. For C₂₇H₂₈N₆F₃SO₂Cl 592.16 mass found 593.1 [M+H]⁺.

### Compound 109: 4-chloro-1-(4-isopropylsulfonylphenyl)sulfonyl-3-[(2R)-2-(trifluoromethyl)pyrrolidin-1-yl]indazole

### Compound 110:4-chloro-1-(4-isopropylsulfonylphenyl)sulfonyl-3-[(2S)-2-(trifluoromethyl)pyrrolidin-1-yl]indazole

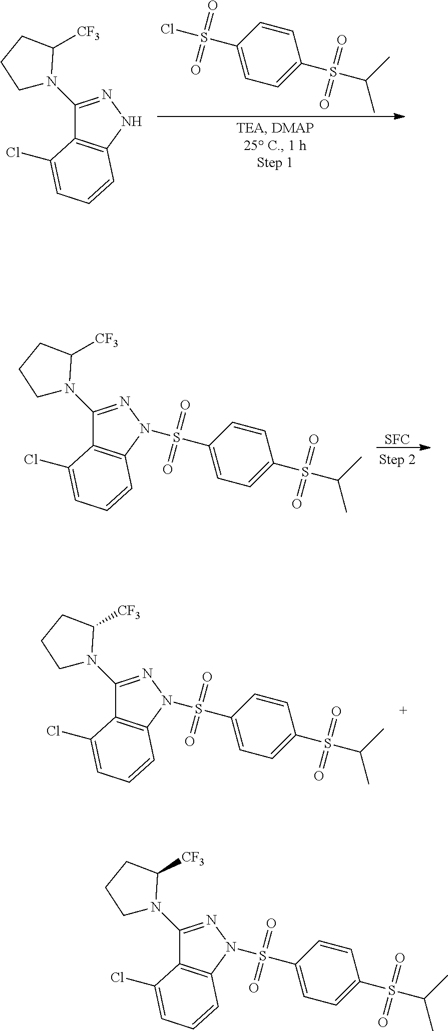

Step 1: To a solution of 4-chloro-3-[2-(trifluoromethyl) pyrrolidin-1-yl]-1H-indazole (55 mg, 190 μmol, 1 eq) and 4-isopropylsulfonylbenzenesulfonyl chloride (80.5 mg, 285 μmol, 1.5 eq) in DCM (2 mL) was added TEA (38.4 mg, 380 μmol, 52.9 μL, 2 eq) and DMAP (2.32 mg, 19.0 μmol, 0.1 eq). The mixture was stirred at 25° C. for 1 hour. LC-MS showed 4-chloro-3-[2-(trifluoromethyl) pyrrolidin-1-yl]-1H-indazole was consumed completely and desired mass was detected. The reaction mixture was filtered and the filter liquor was concentrated under reduced pressure to give a residue. The residue was purified by prep-HPLC (TFA condition; according to LCMS. Method: column: Phenomenex Luna C18 75*30 mm*3 um; mobile phase: [water (TFA)-ACN]; B %: 45%-75%, 8 min to give desired 4-chloro-1-(4-isopropylsulfonylphenyl) sulfonyl-3-[2-(trifluoromethyl) pyrrolidin-1-yl]indazole (100 mg, 181 μmol, 95.5% yield, 97.2% purity) as a white solid. MS (ESI): mass calcd. For C₂₁H₂₁ClF₃N₃O₄S₂ 535.1 m/z found 536.1 [M+H]⁺.

Step 2: 4-chloro-1-(4-isopropylsulfonylphenyl)sulfonyl-3-[2-(trifluoromethyl)pyrrolidin-1-yl]indazole (100 mg, 181 μmol, 1 eq) was separated by chrial SFC column: DAICEL CHIRALPAK AD (250 mm*30 mm, 10 μm); mobile phase: [0.1% NH₃H₂O IPA]; B %: 10%-30%, 12 min) to give two isomers. The structures were assigned randomly. 4-Chloro-1-(4-isopropylsulfonylphenyl) sulfonyl-3-[(2R)-2-(trifluoromethyl)pyrrolidin-1-yl]indazole (9.8 mg, 17.2 μmol, 9.19% yield, 93.80% purity, Rt=2.493 minutes; E.E. by chiral HPLC (%)=95.58%) was isolated as a white solid. ¹H NMR (400 MHz, DMSO-d₆) δ=8.10 (d, J=8.5 Hz, 1H), 8.01 (d, J=8.5 Hz, 4H), 7.68 (s, 1H), 7.52 (d, J=7.8 Hz, 1H), 3.75 (dt, J=5.8, 9.6 Hz, 2H), 3.48 (br s, 1H), 2.43-2.34 (m, 1H), 1.99-1.90 (m, 2H), 1.78 (br s, 1H), 1.23 (s, 1H), 1.07 (dd, J=6.8, 12.1 Hz, 6H). HPLC: 93.80% (220 nm), 94.31% (215 nm), 99.08% (254 nm). MS (ESI): mass calcd. For C₂₁H₂₁ClF₃N₃O₄S₂ 535.1 m/z found 536.1 [M+H]⁺. 4-Chloro-1-(4-isopropylsulfonylphenyl)sulfonyl-3-[(2S)-2-(trifluoromethyl)pyrrolidin-1-yl]indazole (9.7 mg, 17.3 μmol, 9.27% yield, 95.61% purity, Rt=2.653 minutes; E.E. by chiral HPLC (%)=95.18%) was isolated as a white solid. ¹H NMR (400 MHz, DMSO-d₆) δ 8.11 (d, J=8.4 Hz, 1H), 8.02 (q, J=8.6 Hz, 4H), 7.69 (t, J=8.1 Hz, 1H), 7.53 (d, J=7.9 Hz, 1H), 3.84-3.72 (m, 3H), 3.49 (br s, 1H), 1.99-1.90 (m, 2H), 1.83-1.73 (m, 1H), 1.24 (s, 1H), 1.08 (dd, J=6.8, 12.2 Hz, 6H). HPLC: 95.61% (220 nm), 94.81% (215 nm), 99.49% (254 nm). MS (ESI): mass calcd. For C₂₁H₂₁ClF₃N₃O₄S₂ 535.1 m/z found 536.1 [M+H]⁺.

### Compound 111: 4-chloro-3-[7-(5-fluoropyrimidin-2-yl)-4, 7-diazaspiro [2.5]octan-4-yl]-1-(1-isopropylsulfonylpyrrol-3-yl) sulfonyl-indazole

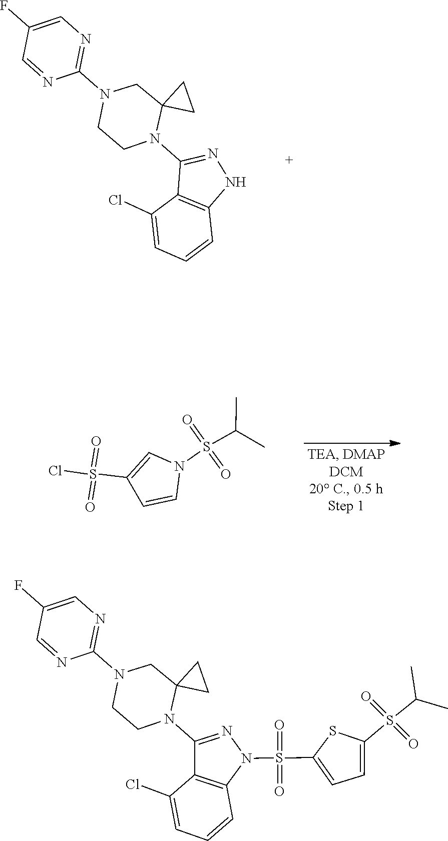 [SMILES: Fc1cnc(N2CCN(c3n[nH]c4cccc(Cl)c34)C3(CC3)C2)nc1 ; CC(C)S(=O)(=O)n1ccc(S(=O)(=O)Cl)c1 ; CC(C)S(=O)(=O)c1ccc(S(=O)(=O)n2nc(N3CCN(c4ncc(F)cn4)CC34CC4)c3c(Cl)cccc32)s1]

Step 1: To a solution of 4-chloro-3-[7-(5-fluoropyrimidin-2-yl)-4, 7-diazaspiro [2.5]octan-4-yl]-1H-indazole (30 mg, 83.6 μmol, 1 eq) and 1-isopropylsulfonylpyrrole-3-sulfonyl chloride (27.3 mg, 100 μmol, 1.2 eq) in DCM (1 mL) was added TEA (21.2 mg, 209 μmol, 2.5 eq) and DMAP (1.02 mg, 8.36 μmol, 0.1 eq). The mixture was stirred at 25° C. for 0.5 hour. LC-MS showed 4-chloro-3-[7-(5-fluoropyrimidin-2-yl)-4, 7-diazaspiro [2.5]octan-4-yl]-1H-indazole was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-HPLC (TFA condition; Method: column: Phenomenex Luna C18 75*30 mm*3 um; mobile phase: [water (TFA)-ACN]; B %: 65%-95%, 8 mins) to give desired compound 4-chloro-3-[7-(5-fluoropyrimidin-2-yl)-4, 7-diazaspiro [2.5]octan-4-yl]-1-(1-isopropylsulfonylpyrrol-3-yl) sulfonyl-indazole (22.9 mg, 38.0 μmol, 45.4% yield, 98.54% purity) as a yellow solid. 1H NMR (400 MHz, DMSO-d₆) δ 8.46 (s, 2H), 8.06-8.00 (m, 1H), 7.90-7.87 (m, 1H), 7.64-7.57 (m, 1H), 7.47-7.43 (m, 1H), 7.35-7.31 (m, 1H), 6.51-6.47 (m, 1H), 3.99-3.85 (m, 3H), 3.75-3.64 (m, 2H), 3.55 (br s, 2H), 1.06-1.00 (m, 6H), 0.83-0.76 (m, 2H), 0.51-0.43 (m, 2H). HPLC: 98.54% (220 nm), 98.66% (215 nm), 98.52% (254 nm). MS (ESI): mass calcd. For C₂₄H₂₅S₂O₄N₇ClF 593.11, m/z found 594.2[M+H]⁺.

### Compound 112: 4-chloro-3-[2-cyclopropyl-4-(5-fluoropyrimidin-2-yl) piperazin-1-yl]-1-[4-(difluoromethyl) phenyl]sulfonyl-indazole

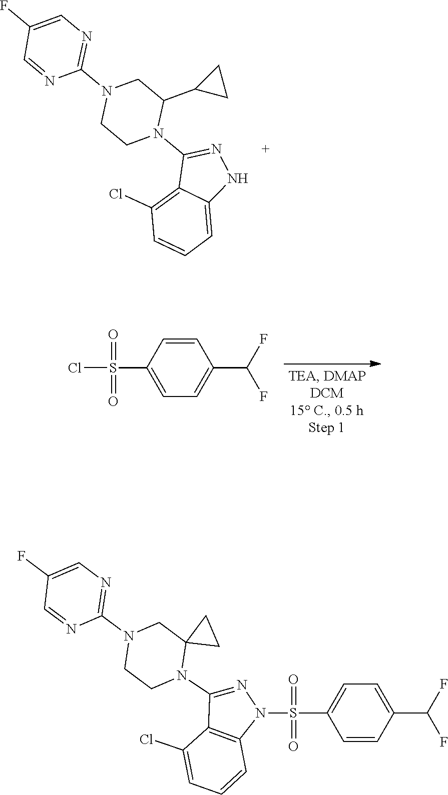 [SMILES: Fc1cnc(N2CCN(c3n[nH]c4cccc(Cl)c34)C(C3CC3)C2)nc1 ; O=S(=O)(Cl)c1ccc(C(F)F)cc1 ; O=S(=O)(c1ccc(C(F)F)cc1)n1nc(N2CCN(c3ncc(F)cn3)CC23CC3)c2c(Cl)cccc21]

Step 1: To a solution of 4-chloro-3-[2-cyclopropyl-4-(5-fluoropyrimidin-2-yl) piperazin-1-yl]-1H-indazole (25 mg, 67.1 μmol, 1 eq) and 4-(difluoromethyl) benzenesulfonyl chloride (45.6 mg, 201 umol, 3 eq) in DCM (1 mL) was added TEA (17.0 mg, 168 umol, 2.5 eq) and DMAP (819 ug, 6.71 umol, 0.1 eq). The mixture was stirred at 25° C. for 0.5 hour. LC-MS showed 4-chloro-3-[2-cyclopropyl-4-(5-fluoropyrimidin-2-yl) piperazin-1-yl]-1H-indazole was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-HPLC (TFA condition; Method: column: Phenomenex Luna C18 75*30 mm*3 um; mobile phase: [water (TFA)-ACN]; B %: 65%-95%, 8 min) to give desired compound 4-chloro-3-[2-cyclopropyl-4-(5-fluoropyrimidin-2-yl) piperazin-1-yl]-1-[4-(difluoromethyl) phenyl]sulfonyl-indazole (7 mg, 11.67 μmol, 17.41% yield, 93.87% purity) as a yellow solid. ¹H NMR (400 MHz, DMSO-d₆) δ 8.51-8.47 (m, 2H), 8.14-8.08 (m, 1H), 8.04-7.98 (m, 2H), 7.78-7.72 (m, 2H), 7.68-7.62 (m, 1H), 7.50-7.44 (m, 1H), 7.22-6.91 (m, 1H), 4.48-4.37 (m, 2H), 3.52-3.47 (m, 2H), 3.31-3.19 (m, 2H), 3.10-3.01 (m, 1H), 1.08-1.02 (m, 1H), 0.23-0.09 (m, 1H), 0.04-−0.08 (m, 1H), −0.24-−0.42 (m, 1H), −0.91 (qd, J=4.9, 9.6 Hz, 1H). HPLC: 93.87% (220 nm), 93.17% (215 nm), 98.36% (254 nm). MS (ESI): mass calcd. For C₂₅H₂₂F₃SO₂NCl 562.12, m/z found 563.2[M+H]⁺.

### Compound 113: 4-chloro-3-[7-(5-fluoropyrimidin-2-yl)-4,7-diazaspiro[2.5]octan-4-yl]-1-[4-(trifluoromethyl)phenyl]sulfonyl-indazole

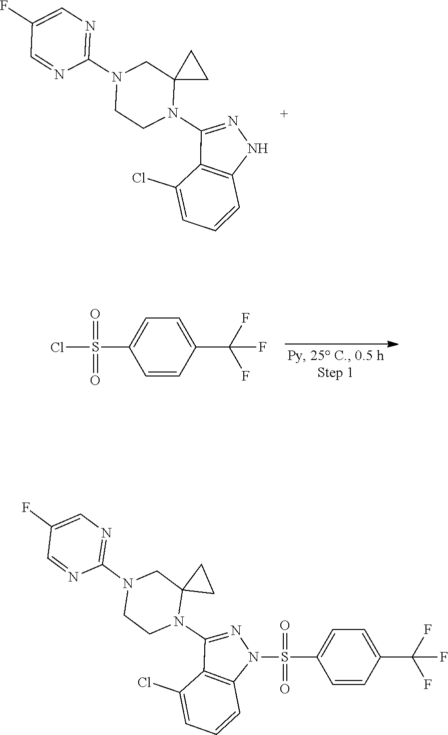 [SMILES: Fc1cnc(N2CCN(c3n[nH]c4cccc(Cl)c34)C3(CC3)C2)nc1 ; O=S(=O)(Cl)c1ccc(C(F)(F)F)cc1 ; O=S(=O)(c1ccc(C(F)(F)F)cc1)n1nc(N2CCN(c3ncc(F)cn3)CC23CC3)c2c(Cl)cccc21]

Step 1: To a solution of 4-chloro-3-[7-(5-fluoropyrimidin-2-yl)-4,7-diazaspiro[2.5]octan-4-yl]-1H-indazole (50.0 mg, 139 μmol, 1 eq) and 4-(trifluoromethyl)benzenesulfonyl chloride (68.2 mg, 278 μmol, 2 eq) in Py (2.00 mL). The mixture was stirred at 25° C. for 0.5 hour. LC-MS showed 4-chloro-3-[7-(5-fluoropyrimidin-2-yl)-4, 7-diazaspiro [2.5]octan-4-yl]-1H-indazole was consumed completely and one main peak with desired m/z detected. The reaction mixture was added to water (10.0 mL), extracted with EtOAc (10 mL*3). The combined organic layers were washed with brine 20.0 mL and dried over Na₂SO₄, filtered and concentrated under reduced pressure to give a residue. The residue was purified by prep-HPLC (TFA condition) to give desired 4-chloro-3-[7-(5-fluoropyrimidin-2-yl)-4, 7-diazaspiro [2.5]octan-4-yl]-1-[4-(trifluoromethyl) phenyl]sulfonyl-indazole (33 mg, 41.65% yield, 99.72% purity) as a light yellow solid. ¹H NMR (400 MHz, DMSO-d₆) δ 8.46 (d, J=0.6 Hz, 2H), 8.10-8.05 (m, 1H), 8.03-7.97 (m, 2H), 7.94-7.89 (m, 2H), 7.65 (t, J=8.1 Hz, 1H), 7.49-7.44 (m, 1H), 3.93-3.88 (m, 2H), 3.58-3.50 (m, 4H), 0.77 (s, 2H), 0.37 (br s, 2H). HPLC: 98.31% (220 nm), 98.26% (215 nm), 99.72% (254 nm). MS (ESI): mass calcd. For C₂₄H₁₉F₄SO₂N₆Cl 566.09 m/z found 567.2 [M+H]⁺.

### Compound 114: 4-chloro-1-[4-(difluoromethylsulfonyl)phenyl]sulfonyl-3-[7-(5-fluoropyrimidin-2-yl)-4,7-diazaspiro[2.5]octan-4-yl]indazole

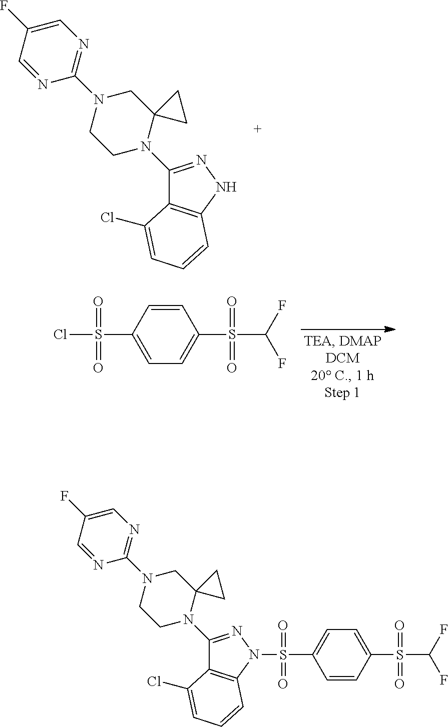

Step 1: To a solution of 4-(difluoromethylsulfonyl)benzenesulfonyl chloride (97.2 mg, 334 μmol, 4 eq) and 4-chloro-3-[7-(5-fluoropyrimidin-2-yl)-4, 7-diazaspiro [2.5]octan-4-yl]-1H-indazole (30.0 mg, 83.6 μmol, 1 eq) in DCM (2 mL) was added dropwise TEA (42.3 mg, 418 μmol, 58.2 μL, 5 eq) and DMAP (1.02 mg, 8.36 μmol, 0.1 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was concentrated to give the crude product. The residue was purified by prep-HPLC (neutral condition; column: Waters Xbridge BEH C18 100*30 mm*10 um; mobile phase: [water(NH₄HCO₃)-ACN]; B %: 55%-85%, 8 min) to give desired 4-chloro-1-[4-(difluoromethylsulfonyl)phenyl]sulfonyl-3-[7-(5-fluoropyrimidin-2-yl)-4,7-diazaspiro[2.5]octan-4-yl]indazole (6.2 mg, 10.1 μmol, 12.1% yield, 99.68% purity) as a yellow solid. ¹H NMR (400 MHz, DMSO-d₆) δ 8.46 (s, 2H), 8.16 (s, 4H), 8.09 (d, J=8.5 Hz, 1H), 7.68 (t, J=8.1 Hz, 1H), 7.52-7.47 (m, 1H), 7.37-7.20 (m, 1H), 3.92 (s, 2H), 3.71-3.57 (m, 2H), 3.55 (br s, 2H), 0.76 (s, 2H), 0.34 (br s, 2H). HPLC: 99.68% (220 nm), 99.67% (210 nm), 99.86% (254 nm). MS (ESI): mass calcd. For C₂₄H₂₀ClF₃N₆O₄S₂ 612.06, m/z found 613.0 [M+H]⁺.

### Compound 115: 4-chloro-1-[4-(difluoromethoxy)phenyl]sulfonyl-3-[7-(5-fluoropyrimidin-2-yl)-4,7-diazaspiro[2.5]octan-4-yl]indazole

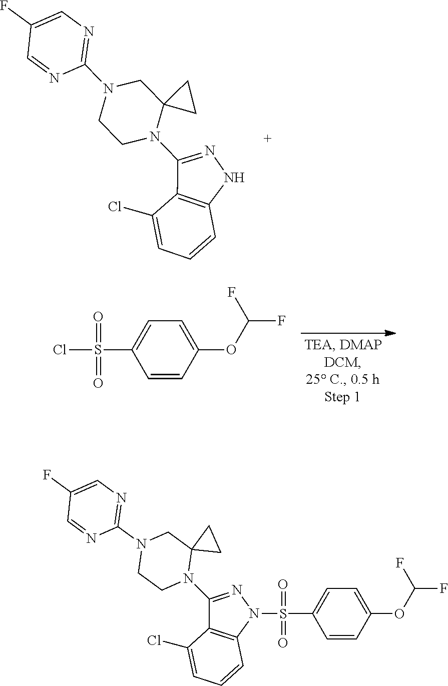 [SMILES: Fc1cnc(N2CCN(c3n[nH]c4cccc(Cl)c34)C3(CC3)C2)nc1 ; O=S(=O)(Cl)c1ccc(OC(F)F)cc1 ; O=S(=O)(c1ccc(OC(F)F)cc1)n1nc(N2CCN(c3ncc(F)cn3)CC23CC3)c2c(Cl)cccc21]

Step 1: To a solution of 4-chloro-3-[7-(5-fluoropyrimidin-2-yl)-4,7-diazaspiro[2.5]octan-4-yl]-1H-indazole (50.0 mg, 139 μmol, 1 eq) and 4-(difluoromethoxy)benzenesulfonyl chloride (67.6 mg, 278 umol, 2 eq) in DCM (2 mL) was added TEA (28.2 mg, 278 umol, 38.7 uL, 2 eq) and DMAP (1.70 mg, 13.9 μmol, 0.1 eq). The mixture was stirred at 25° C. for 0.5 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was added to water (10.0 mL), extracted with EtOAc (10 mL*3). The combined organic layers were washed with brine 20.0 mL and dried over Na₂SO₄, filtered and concentrated under reduced pressure to give a residue. The residue was purified by prep-HPLC (TFA condition:column: Phenomenex Luna C18 75*30 mm*3 um; mobile phase: [water(TFA)-ACN]; B %: 60%-90%, 8 min) to give 4-chloro-1-[4-(difluoromethoxy)phenyl]sulfonyl-3-[7-(5-fluoropyrimidin-2-yl)-4,7-diazaspiro[2.5]octan-4-yl]indazole (37.0 mg, 47.00% yield, 100.00% purity) as a white solid. ¹H NMR (400 MHz, DMSO-d₆) δ 8.49-8.44 (m, 2H), 8.09-8.03 (m, 1H), 7.89-7.83 (m, 2H), 7.66-7.59 (m, 1H), 7.48-7.43 (m, 1H), 7.31-7.26 (m, 2H), 3.99-3.88 (m, 2H), 3.70-3.52 (m, 5H), 0.83-0.74 (m, 2H), 0.45-0.36 (m, 2H). HPLC: 98.45% (220 nm), 98.28% (215 nm), 100.00% (254 nm). MS (ESI): mass calcd. For C₂₄H₂₀O₃SN₆ClF₃ 564.1 m/z found 565.1 [M+H]⁺.

### Compound 116: 4-chloro-1-[4-(1,1-difluoroethyl)phenyl]sulfonyl-3-[7-(5-fluoropyrimidin-2-yl)-4,7-diazaspiro[2.5]octan-4-yl]indazole

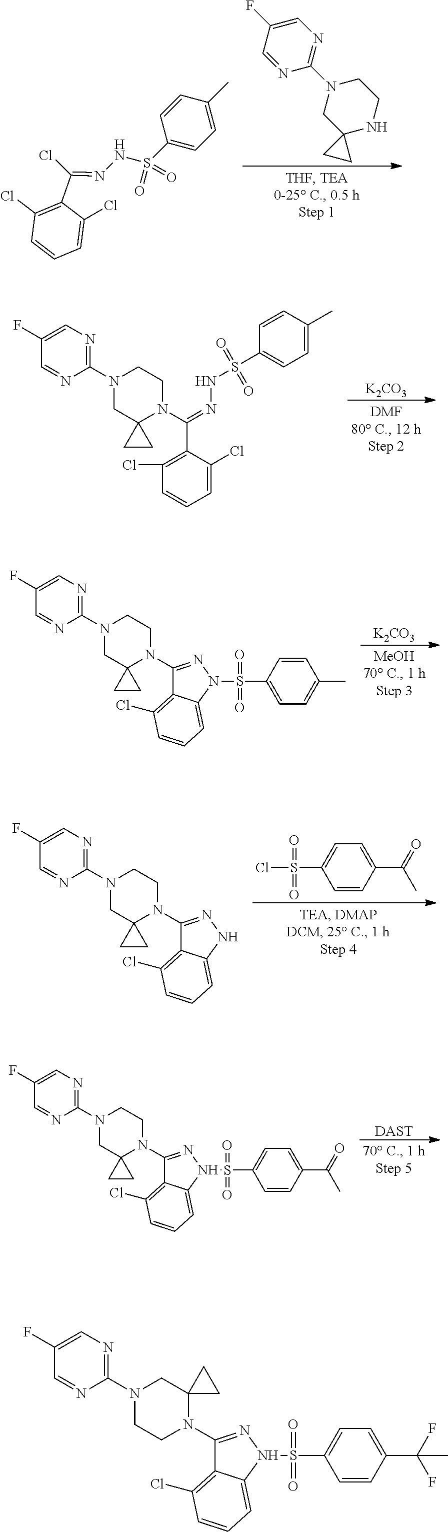

Step 1: To a solution of 7-(5-fluoropyrimidin-2-yl)-4,7-diazaspiro[2.5]octane (778 mg, 3.18 mmol, 1.5 eq, HCl) in THF (5 mL) was added dropwise TEA (2.14 g, 21.2 mmol, 2.95 mL, 10 eq) at 25° C. After addition, the mixture was stirred at this temperature for 10 mins, and then (1Z)-2,6-dichloro-N-(p-tolylsulfonyl)benzohydrazonoyl chloride (800 mg, 2.12 mmol, 1 eq) in THF (3 mL) was added dropwise at 0° C. The resulting mixture was stirred at 25° C. for 20 mins. LC-MS showed 7-(5-fluoropyrimidin-2-yl)-4, 7-diazaspiro [2.5]octane was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N—[(Z)-[(2, 6-dichlorophenyl)-[7-(5-fluoropyrimidin-2-yl)-4, 7-diazaspiro [2.5]octan-4-yl]methylene]amino]-4-methyl-benzenesulfonamide (1.35 g, crude) as a white solid. MS (ESI): mass calcd. For C₂₄H₂₃Cl₂FN₆O₂S 548.10, m/z found 549.1 [M+H]⁺.

Step 2: To a solution of N—[(Z)-[(2, 6-dichlorophenyl)-[7-(5-fluoropyrimidin-2-yl)-4, 7-diazaspiro [2.5]octan-4-yl]methylene]amino]-4-methyl-benzenesulfonamide (1.3 g, 2.37 mmol, 1 eq) in DMF (14 mL) was added K₂CO₃ (1.63 g, 11.8 mmol, 5 eq). The mixture was stirred at 80° C. for 12 hours. LC-MS showed N—[(Z)-[(2, 6-dichlorophenyl)-[7-(5-fluoropyrimidin-2-yl)-4, 7-diazaspiro [2.5]octan-4-yl]methylene]amino]-4-methyl-benzenesulfonamide was consumed completely and desired mass was detected. The reaction mixture was partitioned between 20 mL of H₂O and 20 mL of EtOAc. The organic phase was separated, washed with 50 mL of brine, dried over Na₂SO₄, filtered and concentrated under reduced pressure to give desired 4-chloro-3-[7-(5-fluoropyrimidin-2-yl)-4, 7-diazaspiro [2.5]octan-4-yl]-1-(p-tolylsulfonyl) indazole (1.5 g, crude) as a yellow oil. MS (ESI): mass calcd. For C₂₄H₂₂ClFN₆O₂S 512.12, m/z found 513.0 [M+H]⁺.

Step 3: To a solution of 4-chloro-3-[7-(5-fluoropyrimidin-2-yl)-4, 7-diazaspiro [2.5]octan-4-yl]-1-(p-tolylsulfonyl) indazole (1.5 g, 2.92 mmol, 1 eq) in MeOH (6 mL) was added K₂CO₃ (2.02 g, 14.6 mmol, 5 eq). The mixture was stirred at 70° C. for 1 hour. LC-MS showed 4-chloro-3-[7-(5-fluoropyrimidin-2-yl)-4, 7-diazaspiro [2.5]octan-4-yl]-1-(p-tolylsulfonyl) indazole was consumed completely and desired mass was detected. The reaction mixture was filtered and the filter liquor was concentrated under reduced pressure to give a residue. The residue was purified by flash silica gel chromatography (ISCO®; 2 g SepaFlash® Silica Flash Column, Eluent of 0˜28% Ethyl acetate/petroleum ether gradient @60 mL/min) to give desired 4-chloro-3-[7-(5-fluoropyrimidin-2-yl)-4, 7-diazaspiro [2.5]octan-4-yl]-1H-indazole (250 mg, 697 μmol, 23.8% yield) as a yellow oil. MS (ESI): mass calcd. For C₁₇H₁₆ClFN₆ 358.1, m/z found 359.1 [M+H]⁺.

Step 4: To a solution of 4-chloro-3-[7-(5-fluoropyrimidin-2-yl)-4, 7-diazaspiro [2.5]octan-4-yl]-1H-indazole (500 mg, 1.39 mmol, 1 eq) and 4-acetylbenzenesulfonyl chloride (457 mg, 2.09 mmol, 1.5 eq) in DCM (2 mL) was added TEA (141 mg, 1.39 mmol, 194 μL, 1 eq) and DMAP (170 mg, 1.39 mmol, 1 eq). The mixture was stirred at 25° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was partitioned between 30 mL of H₂O and 50 mL of EtOAc. The organic phase was separated, washed with 50 mL of brine, dried over Na₂SO₄, filtered and concentrated under reduced pressure to give the crude product. The residue was purified by flash silica gel chromatography (ISCO®; 1 g SepaFlash® Silica Flash Column, Eluent of 0˜25% Ethylacetate/petroleum ethergradient @60 mL/min) to give desired 1-[4-[4-chloro-3-[7-(5-fluoropyrimidin-2-yl)-4,7-diazaspiro[2.5]octan-4-yl]indazol-1-yl]sulfonylphenyl]ethanone (750 mg, 1.39 mmol, 99.48% yield) as a yellow oil. MS (ESI): mass calcd. For C₂₅H₂₂ClFN₆O₃S 540.1 m/z found 541.1 [M+H]⁺.

Step 5: To a solution of 1-[4-[4-chloro-3-[7-(5-fluoropyrimidin-2-yl)-4, 7-diazaspiro [2.5]octan-4-yl]indazol-1-yl]sulfonylphenyl]ethanone (750 mg, 1.39 mmol, 1 eq) in DAST (8 mL). The mixture was stirred at 70° C. for 1 hour. LC-MS showed 1-[4-[4-chloro-3-[7-(5-fluoropyrimidin-2-yl)-4, 7-diazaspiro [2.5]octan-4-yl]indazol-1-yl]sulfonylphenyl]ethanone was consumed completely and desired mass was detected. The residue was diluted with sat.NaHCO₃ (60 mL) and extracted with Ethyl acetate 120 mL (40 mL*3). The combined organic layers were washed with brine (60 mL), dried over Na₂SO₄, filtered and concentrated under reduced pressure to give a residue. The residue was purified by prep-HPLC (TFA condition; according to LCMS; Method:column: Phenomenex Luna C18 75*30 mm*3 um; mobile phase: [water(TFA)-ACN]; B %: 60%-90%, 8 min) to give desired 4-chloro-1-[4-(1,1-difluoroethyl)phenyl]sulfonyl-3-[7-(5-fluoropyrimidin-2-yl)-4,7-diazaspiro[2.5]octan-4-yl]indazole as a brown solid. ¹HNMR (400 MHz, DMSO-d₆) δ 8.47 (s, 2H), 8.09 (d, J=8.3 Hz, 1H), 7.90 (d, J=8.5 Hz, 2H), 7.71 (d, J=8.5 Hz, 2H), 7.65 (t, J=8.1 Hz, 1H), 7.48 (d, J=7.5 Hz, 1H), 3.93 (s, 2H), 3.54 (br s, 4H), 1.90 (t, J=19.1 Hz, 3H), 0.78 (s, 2H), 0.39 (br s, 2H). HPLC: 96.57% (220 nm), 96.99% (215 nm), 98.36% (254 nm). MS (ESI): mass calcd. For C₂₅H₂₂ClF₃N₆O₂S 562.1 m/z found 563.1 [M+H]⁺.

### Compound 117: 4-chloro-3-[7-(5-fluoropyrimidin-2-yl)-4, 7-diazaspiro [2.5]octan-4-yl]-1-[4-(trifluoromethoxy) phenyl]sulfonyl-indazole

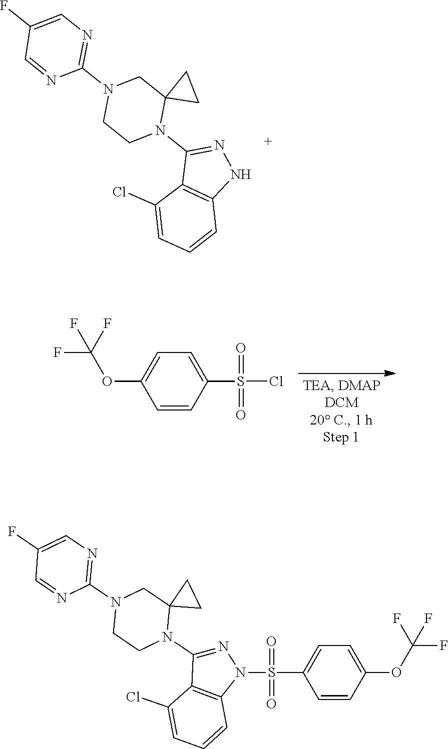 [SMILES: Fc1cnc(N2CCN(c3n[nH]c4cccc(Cl)c34)C3(CC3)C2)nc1 ; O=S(=O)(Cl)c1ccc(OC(F)(F)F)cc1 ; O=S(=O)(c1ccc(OC(F)(F)F)cc1)n1nc(N2CCN(c3ncc(F)cn3)CC23CC3)c2c(Cl)cccc21]

Step 1: To a solution of 4-chloro-3-[7-(5-fluoropyrimidin-2-yl)-4, 7-diazaspiro [2.5]octan-4-yl]-1H-indazole (50 mg, 139 μmol, 1 eq) and 4-(trifluoromethoxy)benzenesulfonyl chloride (182 mg, 697 μmol, 118 μL, 5 eq) in DCM (2 mL) was added dropwise TEA (70.5 mg, 697 μmol, 97.0 μL, 5 eq) and DMAP (1.70 mg, 13.9 μmol, 0.1 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was concentrated to give the crude product. The residue was purified by prep-HPLC (TFA condition; column: Phenomenex Luna C18 75*30 mm*3 um; mobile phase: [water(TFA)-ACN]; B %: 65%-95%, 8 min) to give desired 4-chloro-3-[7-(5-fluoropyrimidin-2-yl)-4, 7-diazaspiro [2.5]octan-4-yl]-1-[4-(trifluoromethoxy) phenyl]sulfonyl-indazole (30.2 mg, 50.7 μmol, 36.4% yield, 97.80% purity) as a yellow solid. ¹H NMR (400 MHz, DMSO-d₆) δ 8.49 (s, 2H), 8.10 (d, J=8.4 Hz, 1H), 7.98-7.91 (m, 2H), 7.67 (t, J=8.1 Hz, 1H), 7.55 (d, J=8.3 Hz, 2H), 7.49 (d, J=7.6 Hz, 1H), 3.94 (s, 2H), 3.56 (br s, 4H), 0.79 (s, 2H), 0.40 (br s, 2H). HPLC: 97.80% (220 nm), 97.95% (210 nm), 97.80% (254 nm). MS (ESI): mass calcd. For C₂₄H₁₉ClF₄N₆O₃S 582.09, m/z found 583.0 [M+H]⁺.

### Compound 118: 4-chloro-3-[7-(5-fluoropyrimidin-2-yl)-4,7-diazaspiro[2.5]octan-4-yl]-1-[4-(trifluoromethylsulfanyl)phenyl]sulfonyl-indazole

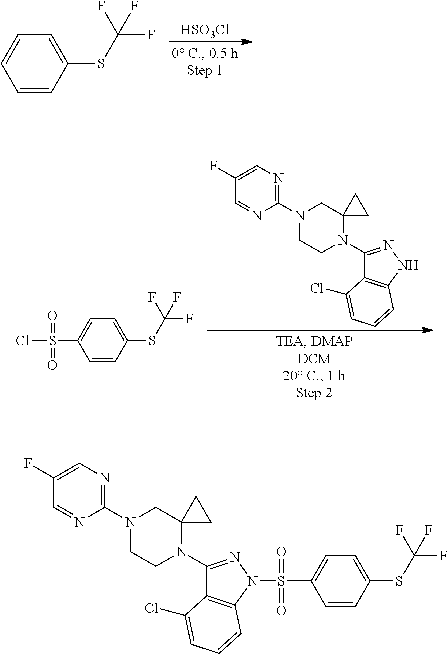

Step 1: To a solution of trifluoromethylsulfanylbenzene (500 mg, 2.81 mmol, 400 μL, 1 eq) in HSO₃Cl (9.16 g, 78.6 mmol, 5.23 mL, 28 eq) at 0° C. The mixture was stirred at 0° C. for 0.5 hour. TLC (petroleum ether/Ethyl acetate=10/1) indicated trifluoromethylsulfanylbenzene was consumed completely and new spot formed. The reaction was clean according to TLC. The reaction mixture was added to water (20 mL), extracted with DCM (10 mL*3). The combined organic layers were washed with brine 20 mL and dried over Na₂SO₄, filtered and concentrated under reduced pressure to give a residue. The crude product 4-(trifluoromethylsulfanyl) benzenesulfonyl chloride (650 mg, crude) as yellow oil was used into the next step without further purification.

Step 2: To a solution of 4-chloro-3-[7-(5-fluoropyrimidin-2-yl)-4, 7-diazaspiro [2.5]octan-4-yl]-1H-indazole (50 mg, 139 μmol, 1 eq) and 4-(trifluoromethylsulfanyl) benzenesulfonyl chloride (154 mg, 557 μmol, 4 eq) in DCM (2 mL) was added dropwise TEA (70.5 mg, 697 μmol, 97.0 μL, 5 eq) and DMAP (1.70 mg, 13.9 μmol, 0.1 eq). The mixture was stirred at 20° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was concentrated to give the crude product. The residue was purified by prep-HPLC (TFA condition; column: Phenomenex Luna C18 75*30 mm*3 um; mobile phase: [water(TFA)-ACN]; B %: 65%-95%, 8 min) to give desired 4-chloro-3-[7-(5-fluoropyrimidin-2-yl)-4, 7-diazaspiro [2.5]octan-4-yl]-1-[4-(trifluoromethylsulfanyl) phenyl]sulfonyl-indazole (26.1 mg, 40.6 μmol, 29.1% yield, 93.14% purity) as a yellow solid. ¹H NMR (400 MHz, DMSO-d₆) δ 8.48 (s, 2H), 8.09 (d, J=8.4 Hz, 1H), 7.95-7.90 (m, 2H), 7.90-7.85 (m, 2H), 7.67 (t, J=8.1 Hz, 1H), 7.49 (d, J=7.7 Hz, 1H), 4.21-3.96 (m, 3H), 3.93 (s, 2H), 3.61 (br d, J=2.7 Hz, 1H), 0.78 (br s, 2H), 0.38 (br s, 2H). HPLC: 93.14% (220 nm), 92.54% (210 nm), 94.81% (254 nm). MS (ESI): mass calcd. For C₂₄H₁₉ClF₄N₆O₂S₂ 598.06, m/z found 599.0 [M+H]⁺.

### Compound 119: 4-chloro-1-((4-(1,1-difluoroethyl)phenyl)sulfonyl)-3-(7-(oxetan-3-yl)-4,7-diazaspiro[2.5]octan-4-yl)-1H-indazole

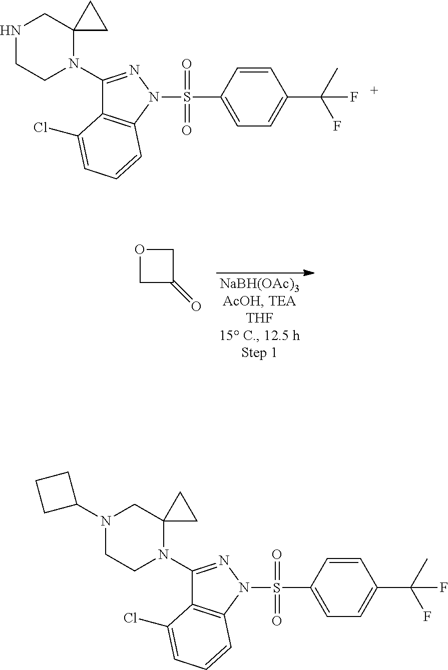 [SMILES: CC(F)(F)c1ccc(S(=O)(=O)n2nc(N3CCNCC34CC4)c3c(Cl)cccc32)cc1 ; O=C1COC1 ; CC(F)(F)c1ccc(S(=O)(=O)n2nc(N3CCN(C4CCC4)CC34CC4)c3c(Cl)cccc32)cc1]

Step 1: To a solution of 4-chloro-3-(4,7-diazaspiro[2.5]octan-4-yl)-1-[4-(1,1-difluoroethyl) phenyl]sulfonyl-indazole (200 mg, 428 μmol, 1 eq) in THF (2 mL) was added TEA (216 mg, 2.14 mmol, 298 μL, 5 eq) and AcOH (51.4 mg, 856 μmol, 49.0 μL, 2 eq) and NaBH(OAc)₃ (136 mg, 642 μmol, 1.5 eq), after 0.5 hour, add oxetan-3-one (21.61 mg, 299 μmol, 0.7 eq) in the mixture. The mixture was stirred at 15° C. for 12 hours. LCMS showed 4-chloro-3-(4, 7-diazaspiro [2.5]octan-4-yl)-1-[4-(1, 1-difluoroethyl) phenyl]sulfonyl-indazole was consumed completely and desired mass was detected. The reaction mixture was added to H₂O (10 mL), extracted with EtOAc (10 mL*3). The combined organic layers were washed with brine (30 mL) and dried over Na₂SO₄, filtered and concentrated under reduced pressure to give a residue. The residue was purified by prep-HPLC (TFA condition: column: Phenomenex Luna C18 75*30 mm*3 μm; mobile phase: [water(TFA)-ACN]; B %: 15%-50%, 8 min) to give desired 4-chloro-1-[4-(1,1-difluoroethyl)phenyl]sulfonyl-3-[7-(oxetan-3-yl)-4,7-diazaspiro[2.5]octan-4-yl]indazole (85 mg, 163 μmol, 37.94% yield, 100.00% purity) as a white solid. ¹H NMR (400 MHz, DMSO-d₆) δ 8.12-8.06 (m, 1H), 7.97-7.89 (m, 2H), 7.83-7.74 (m, 2H), 7.70-7.61 (m, 1H), 7.52-7.44 (m, 1H), 4.92-4.63 (m, 4H), 4.61-4.38 (m, 1H), 3.73-3.59 (m, 3H), 3.39-3.24 (m, 1H), 3.20-2.94 (m, 1H), 2.86-2.60 (m, 1H), 2.03-1.84 (m, 3H), 1.14-0.87 (m, 2H), 0.80-0.59 (m, 1H), 0.45-0.23 (m, 1H) HPLC: 99.80% (220 nm), 99.72% (215 nm), 100.00% (254 nm). MS (ESI): mass calcd. For C₂₄H₂₅N₄F₂SO₃Cl 522.13 m/z found 523.1 [M+H]⁺.

### Compound 120: 4-chloro-1-((4-(1, 1-difluoroethyl) phenyl)sulfonyl)-3-(4-(oxetan-3-yl)-2-(trifluoromethyl)piperazin-1-yl)-1H-indazole

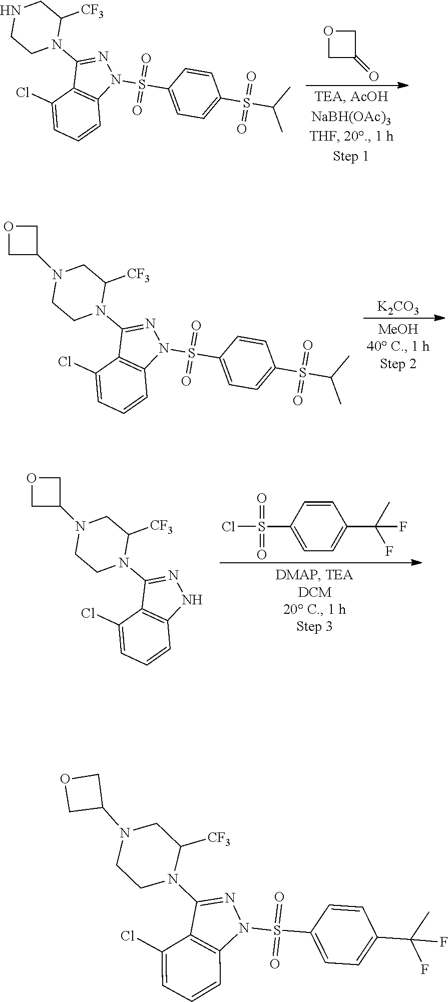

Step 1: To a solution of 4-chloro-1-(4-isopropylsulfonylphenyl)sulfonyl-3-[2-(trifluoromethyl)piperazin-1-yl]indazole (300 mg, 511 μmol, 1 eq, HCl) and oxetan-3-one (25.8 mg, 357 μmol, 0.7 eq) in THF (2 mL) was added TEA (258 mg, 2.55 mmol, 355 μL, 5 eq), AcOH (61.3 mg, 1.02 mmol, 58.5 μL, 2 eq) and NaBH(OAc)₃ (162 mg, 766 μmol, 1.5 eq). The mixture was stirred at 20° C. for 1 hour. LC-MS showed 4-chloro-1-(4-isopropylsulfonylphenyl) sulfonyl-3-[2-(trifluoromethyl) piperazin-1-yl]indazole was not consumed completely and desired mass was detected. The reaction mixture was partitioned between 20 mL of H₂O and 40 mL of EtOAc. The organic phase was separated, washed with 50 mL of brine, dried over Na₂SO₄, filtered and concentrated under reduced pressure to give the crude product. The residue was purified by prep-HPLC (TFA condition; Method: column: Phenomenex Luna C18 75*30 mm*3 μm; mobile phase: [water(TFA)-ACN]; B %: 35%-65%, 8 min) to give desired 4-chloro-1-(4-isopropylsulfonylphenyl)sulfonyl-3-[4-(oxetan-3-yl)-2-(trifluoromethyl)piperazin-1-yl]indazole (70 mg, 104 μmol, 20.3% yield, 90% purity) as a white solid. MS (ESI): mass calcd. For C₂₄H₂₆ClF₃N₄O₅S₂ 606.1, m/z found 607.1 [M+H]⁺.

Step 2: To a solution of 4-chloro-1-(4-isopropylsulfonylphenyl) sulfonyl-3-[4-(oxetan-3-yl)-2-(trifluoromethyl) piperazin-1-yl]indazole (70 mg, 115 μmol, 1 eq) in MeOH (2 mL) was added K₂CO₃ (79.7 mg, 577 μmol, 5 eq). The mixture was stirred at 40° C. for 1 hour. LC-MS showed 4-chloro-1-(4-isopropylsulfonylphenyl) sulfonyl-3-[4-(oxetan-3-yl)-2-(trifluoromethyl) piperazin-1-yl]indazole was consumed completely and desired mass was detected. The reaction mixture was filtered and the filter liquor was concentrated under reduced pressure to give desired 4-chloro-3-[4-(oxetan-3-yl)-2-(trifluoromethyl) piperazin-1-yl]-1H-indazole (40 mg, crude) as a yellow oil. MS (ESI): mass calcd. For C₁₅H₁₆ClF₃N₄O 360.1, m/z found 361.1 [M+H]⁺.

Step 3: To a solution of 4-chloro-3-[4-(oxetan-3-yl)-2-(trifluoromethyl) piperazin-1-yl]-1H-indazole (40 mg, 111 μmol, 1 eq) and 4-(1,1-difluoroethyl)benzenesulfonyl chloride (53.4 mg, 222 μmol, 2 eq) in DCM (2 mL) was added TEA (11.2 mg, 111 μmol, 15.4 μL, 1 eq) and DMAP (13.6 mg, 111 μmol, 1 eq). The mixture was stirred at 20° C. for 1 hour. LC-MS showed 4-chloro-3-[4-(oxetan-3-yl)-2-(trifluoromethyl) piperazin-1-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-HPLC (TFA condition; Method: column: Phenomenex Luna C18 75*30 mm*3 μm; mobile phase: [water(TFA)-ACN]; gradient:50%-80% B over 8 min) to give desired 4-chloro-1-[4-(1,1-difluoroethyl)phenyl]sulfonyl-3-[4-(oxetan-3-yl)-2-(trifluoromethyl)piperazin-1-yl]indazole (15 mg, 26.3 μmol, 23.7% yield, 99.07% purity) as a white solid. ¹H NMR (400 MHz, DMSO-d₆) δ 8.11 (d, J=8.4 Hz, 1H), 7.94 (d, J=8.3 Hz, 2H), 7.75 (d, J=8.4 Hz, 2H), 7.67 (t, J=8.1 Hz, 1H), 7.51 (d, J=7.8 Hz, 1H), 4.58 (td, J=6.6, 15.9 Hz, 3H), 4.51-4.36 (m, 2H), 3.75-3.63 (m, 2H), 3.53 (br d, J=5.6 Hz, 1H), 3.31 (br d, J=12.8 Hz, 1H), 3.04 (br d, J=12.4 Hz, 1H), 2.81-2.72 (m, 1H), 2.56-2.52 (m, 1H), 1.90 (t, J=19.1 Hz, 3H). HPLC: 99.07% (220 nm), 99.32% (215 nm), 99.09% (254 nm). MS (ESI): mass calcd. For C₂₃H₂₂ClF₅N₄O₃S 564.1, m/z found 565.1 [M+H]⁺.

### Compound 121: (S)-4-chloro-1-((4-(1,1-difluoroethyl)phenyl)sulfonyl)-3-(2-(2-fluoropropan-2-yl)-4-(5-fluoropyrimidin-2-yl)piperazin-1-yl)-1H-indazole

### Compound 122: (R)-4-chloro-1-((4-(1,1-difluoroethyl)phenyl)sulfonyl)-3-(2-(2-fluoropropan-2-yl)-4-(5-fluoropyrimidin-2-yl)piperazin-1-yl)-1H-indazole

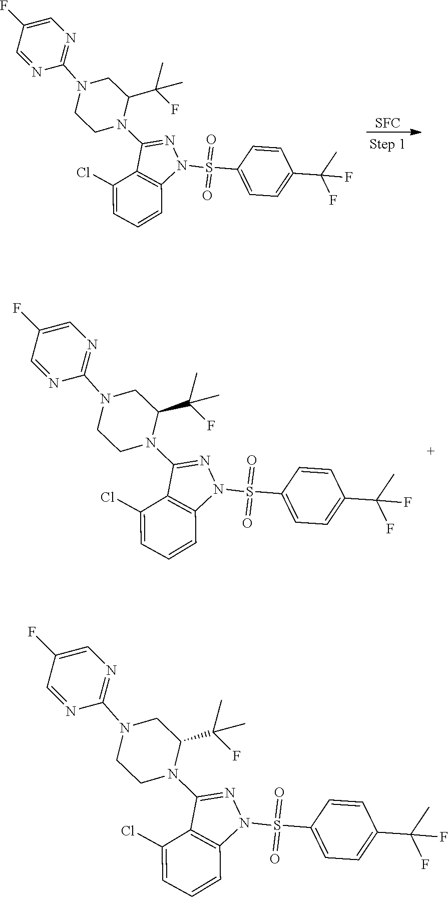

Step 1: The residue was purified by SFC (Method: column: Daicel ChiralPak IG (250*30 mm, 10 μm); mobile phase: [CO₂-i-PrOH (0.10% NH₃·H₂O)]; 60% B isocratic elution mode) to give two isomers. The structures were assigned randomly. 4-Chloro-1-[4-(1, 1-difluoroethyl) phenyl]sulfonyl-3-[(2S)-2-(1-fluoro-1-methyl-ethyl)-4-(5-fluoropyrimidin-2-yl) piperazin-1-yl]indazole (102 mg, 165 μmol, 37.9% yield, 96.83% purity, Rt=1.175 minutes; E.E. by chiral HPLC (%)=100.0%) was isolated as a yellow solid. ¹H NMR (400 MHz, DMSO-d₆) δ 8.49 (s, 2H), 8.10 (d, J=8.1 Hz, 1H), 7.87 (d, J=8.5 Hz, 2H), 7.71-7.64 (m, 3H), 7.51 (d, J=7.4 Hz, 1H), 4.33-4.15 (m, 3H), 3.87-3.70 (m, 2H), 3.52 (br d, J=12.6 Hz, 1H), 3.14 (dt, J=4.1, 12.1 Hz, 1H), 1.90 (t, J=19.1 Hz, 3H), 1.18 (d, J=6.0 Hz, 3H), 1.13 (d, J=6.0 Hz, 3H). HPLC: 96.83% (220 nm), 96.75% (215 nm), 97.21% (254 nm). MS (ESI): mass calcd. For C₂₆H₂₅N₆F₄SO₂Cl 596.14, m/z found 597.1[M+H]⁺. 4-Chloro-1-[4-(1, 1-difluoroethyl) phenyl]sulfonyl-3-[(2R)-2-(1-fluoro-1-methyl-ethyl)-4-(5-fluoropyrimidin-2-yl) piperazin-1-yl]indazole (82.4 mg, 137 μmol, 31.4% yield, 99.02% purity, Rt=1.588 minutes; E.E. by chiral HPLC (%)=100.0%) was isolated as a yellow solid. ¹H NMR (400 MHz, DMSO-d₆) δ 8.49 (s, 2H), 8.09 (d, J=8.3 Hz, 1H), 7.87 (d, J=8.3 Hz, 2H), 7.72-7.63 (m, 3H), 7.51 (d, J=7.7 Hz, 1H), 4.33-4.15 (m, 3H), 3.85-3.70 (m, 2H), 3.52 (br d, J=13.2 Hz, 1H), 3.18-3.07 (m, 1H), 1.89 (t, J=19.1 Hz, 3H), 1.18 (d, J=6.0 Hz, 3H), 1.12 (d, J=6.1 Hz, 3H). HPLC: 99.02% (220 nm), 99.82% (215 nm), 99.82% (254 nm). MS (ESI): mass calcd. For C₂₆H₂₅N₆F₄SO₂Cl 596.14, m/z found 597.1[M+H]⁺.

### Compound 123: 4-chloro-1-((4-(1,1-difluoroethyl)phenyl)sulfonyl)-3-(7-(3,5-difluoropyridin-2-yl)-4,7-diazaspiro[2.5]octan-4-yl)-1H-indazole

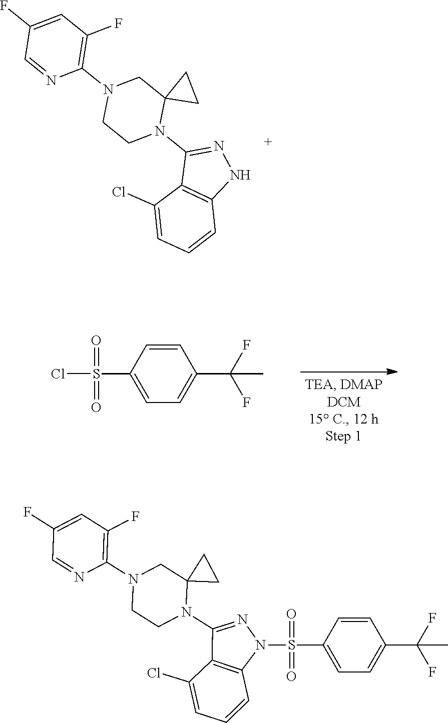 [SMILES: Fc1cnc(N2CCN(c3n[nH]c4cccc(Cl)c34)C3(CC3)C2)c(F)c1 ; CC(F)(F)c1ccc(S(=O)(=O)Cl)cc1 ; CC(F)(F)c1ccc(S(=O)(=O)n2nc(N3CCN(c4ncc(F)cc4F)CC34CC4)c3c(Cl)cccc32)cc1]

Step 1: To a solution of 4-(1,1-difluoroethyl)benzenesulfonyl chloride (230 mg, 958 μmol, 1.2 eq) and 4-chloro-3-[7-(3,5-difluoro-2-pyridyl)-4,7-diazaspiro[2.5]octan-4-yl]-1H-indazole (300 mg, 798 μmol, 1 eq) in DCM (5 mL) was added TEA (161 mg, 1.60 mmol, 222 μL, 2 eq) and DMAP (9.75 mg, 79.8 μmol, 0.1 eq). The mixture was stirred at 15° C. for 12 hours. LCMS showed 4-chloro-3-[7-(3, 5-difluoro-2-pyridyl)-4, 7-diazaspiro [2.5]octan-4-yl]-1H-indazole was consumed completely and desired mass was detected. The reaction mixture was added to H₂O (30 mL), extracted with EtOAc (30 mL*3). The combined organic layers were washed with brine (20 mL) and dried over Na₂SO₄, filtered and concentrated under reduced pressure to give a residue. The residue was purified by prep-HPLC (TFA condition; column: Phenomenex Luna C18 75*30 mm*3 μm; mobile phase: [H₂O(0.1% TFA)-ACN]; gradient: 70%-95% B over 8.0 min) to give desired 4-chloro-1-[4-(1,1-difluoroethyl)phenyl]sulfonyl-3-[7-(3, 5-difluoro-2-pyridyl)-4, 7-diazaspiro [2.5]octan-4-yl]indazole (184 mg, 314 μmol, 39.4% yield, 99.06% purity) as a white solid. ¹H NMR (400 MHz, DMSO-d₆) δ 8.14-8.10 (m, 1H), 8.09-8.04 (m, 1H), 7.91-7.77 (m, 3H), 7.71-7.60 (m, 3H), 7.49-7.44 (m, 1H), 3.64-3.57 (m, 2H), 3.56-3.44 (m, 2H), 3.24-3.03 (m, 2H), 1.93-1.81 (m, 3H), 0.82-0.72 (m, 2H), 0.46-0.34 (m, 2H) HPLC: 98.95% (220 nm), 99.44% (215 nm), 99.06% (254 nm). MS (ESI): mass calcd. For C₂₆H₂₂N₅F₄SO₂Cl 579.11, m/z found 580.0 [M+H]⁺.

### Compound 124: 4-chloro-3-(7-(4-chloro-2-fluorophenyl)-4,7-diazaspiro[2.5]octan-4-yl)-1-((4-(1,1-difluoroethyl)phenyl)sulfonyl)-1H-indazole

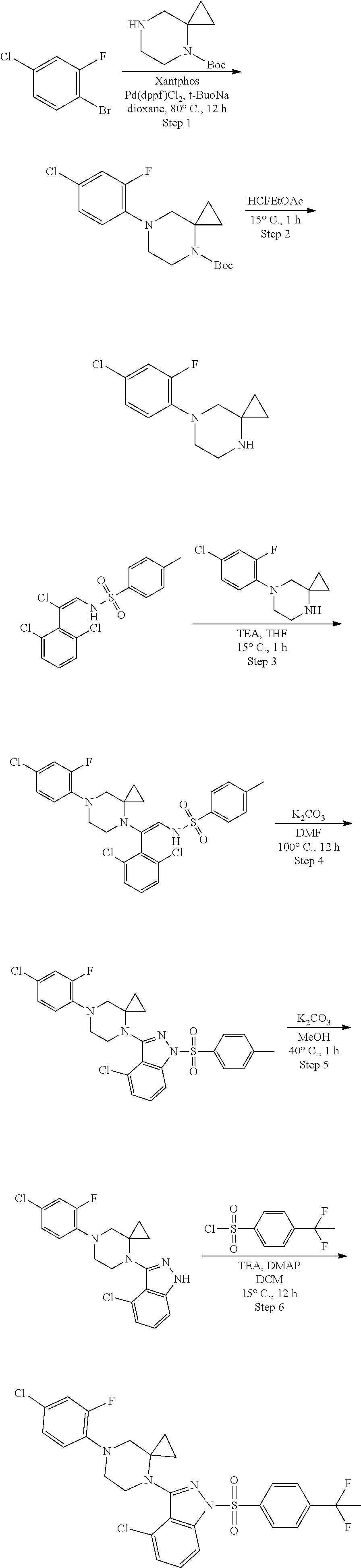

Step 1: A mixture of tert-butyl 4, 7-diazaspiro [2.5]octane-4-carboxylate (1.5 g, 7.07 mmol, 1 eq), 1-bromo-4-chloro-2-fluoro-benzene (1.48 g, 7.07 mmol, 882 μL, 1 eq), Pd(dppf)Cl₂ (517 mg, 707 μmol, 0.1 eq), Xantphos (818 mg, 1.41 mmol, 0.2 eq) and t-BuONa (2.04 g, 21.2 mmol, 3 eq) in dioxane (30 mL) was degassed and purged with N₂ for 3 times, and then the mixture was stirred at 80° C. for 12 hours under N₂ atmosphere. LC-MS showed tert-butyl 4, 7-diazaspiro [2.5]octane-4-carboxylate was consumed completely and desired mass was detected. The reaction mixture was partitioned between 60 mL of H₂O and 80 mL of EtOAc. The organic phase was separated, washed with 50 mL of brine, dried over Na₂SO₄, filtered and concentrated under reduced pressure to give the crude product. The residue was purified by flash silica gel chromatography (ISCO®; 5 g SepaFlash® Silica Flash Column, Eluent of 0˜10% ethyl acetate/petroleum ether gradient @60 mL/min) to give desired tert-butyl 7-(4-chloro-2-fluoro-phenyl)-4, 7-diazaspiro [2.5]octane-4-carboxylate (1.2 g, 3.52 mmol, 49.8% yield) as a yellow oil. MS (ESI): mass calcd. For C₁₇H₂₂ClFN₂O₂ 340.1 m/z found 341.1 [M+H]⁺.

Step 2: To a solution of tert-butyl 7-(4-chloro-2-fluoro-phenyl)-4, 7-diazaspiro [2.5]octane-4-carboxylate (1.2 g, 3.52 mmol, 1 eq) in HCl/EtOAc (4 M, 13.2 mL, 15 eq). The mixture was stirred at 15° C. for 1 hour. LC-MS showed tert-butyl 7-(4-chloro-2-fluoro-phenyl)-4, 7-diazaspiro [2.5]octane-4-carboxylate was consumed completely and desired mass was detected. The reaction mixture was filtered and the filter liquor was concentrated under reduced pressure to give desired 7-(4-chloro-2-fluoro-phenyl)-4, 7-diazaspiro [2.5]octane (950 mg, crude, HCl) as a yellow solid. MS (ESI): mass calcd. For Cl₂H₁₄ClFN₂ 240.1 m/z found 241.1 [M+H]⁺.

Step 3: To a solution of (1E)-2,6-dichloro-N-(p-tolylsulfonyl)benzohydrazonoyl chloride (471 mg, 1.25 mmol, 1.5 eq) and 7-(4-chloro-2-fluoro-phenyl)-4,7-diazaspiro[2.5]octane (200 mg, 831 μmol, 1 eq) in THF (5 mL) was added TEA (420 mg, 4.15 mmol, 578 μL, 5 eq). The mixture was stirred at 15° C. for 1 hour. TLC showed (1E)-2,6-dichloro-N-(p-tolylsulfonyl)benzohydrazonoyl chloride (471 mg, 1.25 mmol, 1.5 eq) was consumed completely and a new spot was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. LC-MS showed 7-(4-chloro-2-fluoro-phenyl)-4, 7-diazaspiro [2.5]octane was consumed completely and desired mass was detected. The reaction mixture was partitioned between water (30 mL) and EtOAc (90 mL). The organic phase was separated, washed with brine (100 mL), dried over Na₂SO₄, filtered and concentrated under reduced pressure to give desired N-[(E)-[[7-(4-chloro-2-fluoro-phenyl)-4,7-diazaspiro[2.5]octan-4-yl]-(2,6-dichlorophenyl)methylene]amino]-4-methyl-benzenesulfonamide (500 mg, crude) as a yellow solid. MS (ESI): mass calcd. For C₂₆H₂₄C₁₃FN₄O₂S 580.1, m/z found 581.1 [M+H]⁺.

Step 4: To a solution of N-[(E)-[[7-(4-chloro-2-fluoro-phenyl)-4, 7-diazaspiro [2.5]octan-4-yl]-(2, 6-dichlorophenyl) methylene]amino]-4-methyl-benzenesulfonamide (500 mg, 859 μmol, 1 eq) in DMF (10 mL) was added K₂CO₃ (594 mg, 4.30 mmol, 5 eq). The mixture was stirred at 100° C. for 12 hours. LC-MS showed N-[(E)-[[7-(4-chloro-2-fluoro-phenyl)-4, 7-diazaspiro [2.5]octan-4-yl]-(2, 6-dichlorophenyl) methylene]amino]-4-methyl-benzenesulfonamide was consumed completely and desired mass was detected. The reaction mixture was partitioned between H₂O (30 mL) and MTBE (20 mL*3). The organic phase was separated, washed with brine (15 mL*2), dried over Na₂SO₄, filtered and concentrated under reduced pressure to give desired 4-chloro-3-[7-(4-chloro-2-fluoro-phenyl)-4,7-diazaspiro[2.5]octan-4-yl]-1-(p-tolylsulfonyl) indazole (800 mg, crude) as a brown solid. MS (ESI): mass calcd. For C₂₆H₂₃Cl₂FN₄O₂S 544.1, m/z found 545.1 [M+H]⁺.

Step 5: To a solution of 4-chloro-3-[7-(4-chloro-2-fluoro-phenyl)-4, 7-diazaspiro [2.5]octan-4-yl]-1-(p-tolylsulfonyl) indazole (800 mg, 1.47 mmol, 1 eq) in MeOH (10 mL) was added K₂CO₃ (1.01 g, 7.33 mmol, 5 eq). The mixture was stirred at 40° C. for 1 hour. LC-MS showed 4-chloro-3-[7-(4-chloro-2-fluoro-phenyl)-4, 7-diazaspiro [2.5]octan-4-yl]-1-(p-tolylsulfonyl) indazole was consumed completely and desired mass was detected. The reaction mixture was partitioned between H₂O (30 mL) and MTBE (20 mL*3). The organic phase was separated, washed with brine (15 mL*2), dried over Na₂SO₄, filtered and concentrated under reduced pressure to give a residue. The residue was purified by prep-TLC (SiO₂, petroleum ether/Ethyl acetate=3/1) to give desired 4-chloro-3-[7-(4-chloro-2-fluoro-phenyl)-4, 7-diazaspiro [2.5]octan-4-yl]-1H-indazole (150 mg, 383 μmol, 26.1% yield) as a white solid. MS (ESI): mass calcd. For C₁₉H₁₇Cl₂FN₄ 390.1, m/z found 391.1 [M+H]⁺.

Step 6: To a solution of 4-(1,1-difluoroethyl)benzenesulfonyl chloride (111 mg, 460 mol, 1.2 eq) and 4-chloro-3-[7-(4-chloro-2-fluoro-phenyl)-4,7-diazaspiro[2.5]octan-4-yl]-1H-indazole (150 mg, 383 μmol, 1 eq) in DCM (2 mL) was added TEA (116 mg, 1.15 mmol, 160 μL, 3 eq) and DMAP (4.68 mg, 38.3 μmol, 0.1 eq). The mixture was stirred at 15° C. for 12 hours. LC-MS showed 4-chloro-3-[7-(4-chloro-2-fluoro-phenyl)-4, 7-diazaspiro [2.5]octan-4-yl]-1H-indazole was consumed completely and desired mass was detected. The reaction mixture was added to water (30 mL), extracted with EtOAc (30 mL*3). The combined organic layers were washed with brine (30 mL) and dried over Na₂SO₄, filtered and concentrated under reduced pressure to give a residue. The residue was purified by prep-HPLC (TFA condition: column: Phenomenex Luna C18 75*30 mm*3 μm; mobile phase: [H₂O(0.1% TFA)-ACN]; gradient: 70%-95% B over 8.0 min) to give desired 4-chloro-3-[7-(4-chloro-2-fluoro-phenyl)-4,7-diazaspiro[2.5]octan-4-yl]-1-[4-(1,1-difluoroethyl)phenyl]sulfonyl-indazole (60 mg, 96.7 μmol, 25.2% yield, 96.96% purity) as a white solid. ¹H NMR (400 MHz, DMSO-d₆) δ 8.12-8.06 (m, 1H), 7.93-7.86 (m, 2H), 7.72-7.61 (m, 3H), 7.52-7.44 (m, 1H), 7.39-7.31 (m, 1H), 7.24-7.16 (m, 1H), 7.10-7.01 (m, 1H), 3.64-3.58 (m, 2H), 3.43-3.39 (m, 4H), 1.90-1.75 (m, 3H), 1.21 (s, 1H), 0.86-0.73 (m, 2H), 0.50-0.43 (m, 1H). HPLC: 96.96% (220 nm), 97.22% (210 nm), 96.21% (254 nm). MS (ESI): mass calcd. For C₂₇H₂₃Cl₂F₃N₄O₂S 594.1, m/z found 595.1 [M+H]⁺.

### Compound 125: 4-chloro-3-(7-(2-chloro-4-fluorophenyl)-4, 7-diazaspiro [2.5]octan-4-yl)-1-((4-(1,1-difluoroethyl)phenyl)sulfonyl)-1H-indazole

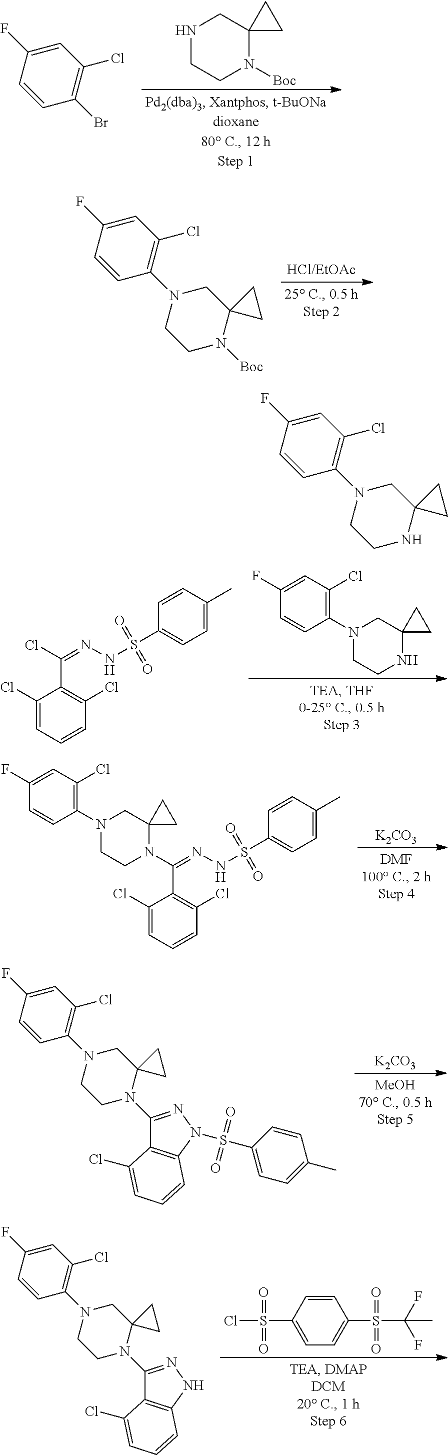

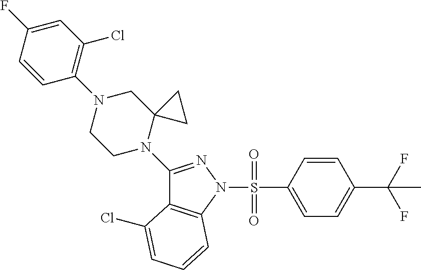 [SMILES: CC(F)(F)c1ccc(S(=O)(=O)n2nc(N3CCN(c4ccc(F)cc4Cl)CC34CC4)c3c(Cl)cccc32)cc1]

Step 1: A mixture of 1-bromo-2-chloro-4-fluoro-benzene (493 mg, 2.36 mmol, 1 eq), tert-butyl 4,7-diazaspiro[2.5]octane-4-carboxylate (500 mg, 2.36 mmol, 1 eq), Pd(dppf)Cl₂ (172 mg, 236 μmol, 0.1 eq), Xantphos (273 mg, 471 μmol, 0.2 eq) and t-BuONa (679 mg, 7.07 mmol, 3 eq) in dioxane (10 mL) was degassed and purged with N₂ for 3 times, and then the mixture was stirred at 80° C. for 12 hours under N₂ atmosphere. LC-MS showed 1-bromo-2-chloro-4-fluoro-benzene was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by column chromatography (SiO₂, petroleum ether/ethyl acetate=1/0 to 1/1) to give desired tert-butyl 7-(2-chloro-4-fluoro-phenyl)-4, 7-diazaspiro [2.5]octane-4-carboxylate (490 mg, crude) as a yellow oil. MS (ESI): mass calcd. For C₁₇H₂₂ClFN₂O₂ 340.14, m/z found 341.1 [M+H]⁺.

Step 2: A mixture of tert-butyl 7-(2-chloro-4-fluoro-phenyl)-4, 7-diazaspiro [2.5]octane-4-carboxylate (490 mg, 1.44 mmol, 1 eq) in HCl/EtOAc (4M, 4 mL) was stirred at 20° C. for 0.5 hour. LCMS showed tert-butyl 7-(2-chloro-4-fluoro-phenyl)-4, 7-diazaspiro [2.5]octane-4-carboxylate was consumed completely and desired mass was detected. The reaction mixture was concentrated to give desired 7-(2-chloro-4-fluoro-phenyl)-4, 7-diazaspiro [2.5]octane (390 mg, crude, HCl) as a yellow solid. MS (ESI): mass calcd. For C₁₂H₁₄ClFN₂ 240.08, m/z found 241.0 [M+H]⁺.

Step 3: To a solution of (1Z)-2, 6-dichloro-N-(p-tolylsulfonyl) benzohydrazonoyl chloride (300 mg, 794 μmol, 1.1 eq) in THF (3 mL) was added dropwise TEA (730 mg, 7.22 mmol, 1.00 mL, 10 eq) at 25° C. After addition, the mixture was stirred at this temperature for 10 minutes, and then 7-(2-chloro-4-fluoro-phenyl)-4, 7-diazaspiro [2.5]octane (200 mg, 721.59 μmol, 1 eq, HCl) in THF (3 mL) was added dropwise at 0° C. The resulting mixture was stirred at 25° C. for 20 minutes. LC-MS showed (1Z)-2, 6-dichloro-N-(p-tolylsulfonyl) benzohydrazonoyl chloride was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N-[(E)-[[7-(2-chloro-4-fluoro-phenyl)-4, 7-diazaspiro [2.5]octan-4-yl]-(2, 6-dichlorophenyl) methylene]amino]-4-methyl-benzenesulfonamide (400 mg, crude) as a yellow solid. MS (ESI): mass calcd. For C₂₆H₂₄C₁₃FN₄O₂S 580.07, m/z found 581.1 [M+H]⁺.

Step 4: To a solution of N-[(E)-[[7-(2-chloro-4-fluoro-phenyl)-4, 7-diazaspiro [2.5]octan-4-yl]-(2, 6-dichlorophenyl) methylene]amino]-4-methyl-benzenesulfonamide (400 mg, 687 μmol, 1 eq) in DMF (5 mL) was added K₂CO₃ (475 mg, 3.44 mmol, 5 eq). The mixture was stirred at 100° C. for 2 hours. LC-MS showed N-[(E)-[[7-(2-chloro-4-fluoro-phenyl)-4, 7-diazaspiro [2.5]octan-4-yl]-(2, 6-dichlorophenyl) methylene]amino]-4-methyl-benzenesulfonamide was consumed completely and desired mass was detected. The reaction mixture was added to water (20 mL), extracted with EtOAc (20 mL*3). The combined organic layers were washed with brine (20 mL) and dried over Na₂SO₄, filtered and concentrated under reduced pressure to give desired 4-chloro-3-[7-(2-chloro-4-fluoro-phenyl)-4, 7-diazaspiro [2.5]octan-4-yl]-1-(p-tolylsulfonyl) indazole (370 mg, crude) as an orange oil. MS (ESI): mass calcd. For C₂₆H₂₃Cl₂FN₄O₂S 544.09, m/z found 545.1 [M+H]⁺.

Step 5: To a solution of 4-chloro-3-[7-(2-chloro-4-fluoro-phenyl)-4, 7-diazaspiro [2.5]octan-4-yl]-1-(p-tolylsulfonyl) indazole (370 mg, 678 μmol, 1 eq) in MeOH (5 mL) was added K₂CO₃ (1.88 g, 13.6 mmol, 20 eq). The mixture was stirred at 70° C. for 0.5 hour. LC-MS showed 4-chloro-3-[7-(2-chloro-4-fluoro-phenyl)-4, 7-diazaspiro [2.5]octan-4-yl]-1-(p-tolylsulfonyl) indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (SiO₂, petroleum ether/ethyl acetate=2/1) to give desired 4-chloro-3-[7-(2-chloro-4-fluoro-phenyl)-4, 7-diazaspiro [2.5]octan-4-yl]-1H-indazole (120 mg, crude) as an orange oil. MS (ESI): mass calcd. For C₁₉H₁₇Cl₂FN₄ 390.08, m/z found 391.1 [M+H]⁺.

Step 6: To a solution of 4-(1,1-difluoroethyl)benzenesulfonyl chloride (111 mg, 460 μmol, 1.5 eq) and 4-chloro-3-[7-(2-chloro-4-fluoro-phenyl)-4, 7-diazaspiro [2.5]octan-4-yl]-1H-indazole (120 mg, 307 μmol, 1 eq) in DCM (2 mL) was added TEA (93.1 mg, 920 μmol, 128 μL, 3 eq) and DMAP (3.75 mg, 30.7 μmol, 0.1 eq). The mixture was stirred at 20° C. for 1 hour. LC-MS showed 4-chloro-3-[7-(2-chloro-4-fluoro-phenyl)-4, 7-diazaspiro [2.5]octan-4-yl]-1H-indazole was consumed completely and one main peak with desired mass was detected. The reaction mixture was concentrated to give the crude product. The residue was purified by prep-HPLC (TFA condition; column: Phenomenex Luna C18 75*30 mm*3 μm; mobile phase: [H₂O (0.10% TFA)-ACN]; gradient: 75%-95% B over 8.0 min) to give desired 4-chloro-3-[7-(2-chloro-4-fluoro-phenyl)-4, 7-diazaspiro [2.5]octan-4-yl]-1-[4-(1, 1-difluoroethyl) phenyl]sulfonyl-indazole (17.4 mg, 29.2 μmol, 9.52% yield, 99.95% purity) as a black brown solid. ¹H NMR (400 MHz, DMSO-d₆) δ 8.09 (d, J=8.3 Hz, 1H), 7.91 (d, J=8.4 Hz, 2H), 7.72 (d, J=8.4 Hz, 2H), 7.64 (t, J=8.1 Hz, 1H), 7.47 (d, J=7.6 Hz, 1H), 7.42 (br d, J=8.3 Hz, 1H), 7.18 (d, J=5.6 Hz, 2H), 3.60 (br s, 2H), 3.38-3.30 (m, 2H), 2.92-2.60 (m, 2H), 1.84 (t, J=19.1 Hz, 3H), 0.84 (br s, 2H), 0.58-0.33 (m, 2H). HPLC: 99.95% (220 nm), 99.97% (210 nm), 100.00% (254 nm). MS (ESI): mass calcd. For C₂₇H₂₃Cl₂F₃N₄O₂S 594.09, m/z found 595.1 [M+H]⁺.

### Compound 126: 4-chloro-1-((4-(1,1-difluoroethyl)phenyl)sulfonyl)-3-(7-(2,4-difluorophenyl)-4,7-diazaspiro[2.5]octan-4-yl)-1H-indazole

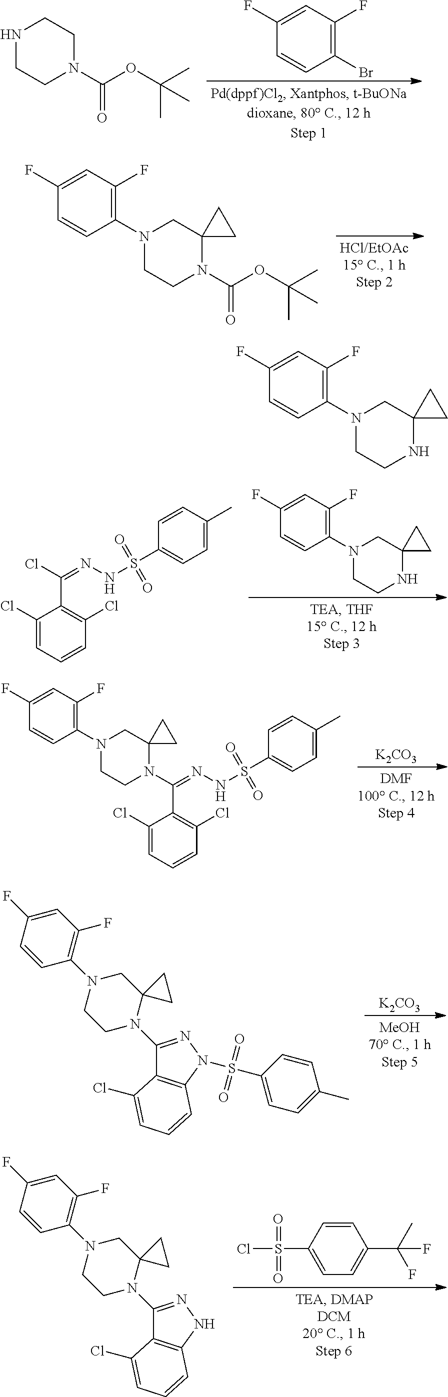

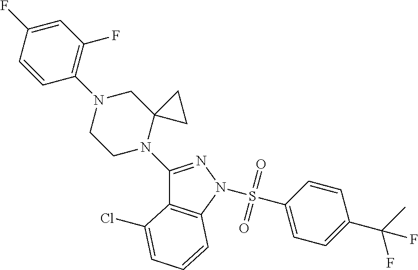 [SMILES: CC(F)(F)c1ccc(S(=O)(=O)n2nc(N3CCN(c4ccc(F)cc4F)CC34CC4)c3c(Cl)cccc32)cc1]

Step 1: A mixture of tert-butyl 4,7-diazaspiro[2.5]octane-4-carboxylate (500 mg, 2.36 mmol, 1 eq), 1-bromo-2,4-difluoro-benzene (455 mg, 2.36 mmol, 266 μL, 1 eq), Pd(dppf)Cl₂ (172 mg, 236 μmol, 0.1 eq), Xantphos (273 mg, 471 μmol, 0.2 eq) and t-BuONa (679 mg, 7.07 mmol, 3 eq) in dioxane (10 mL) was degassed and purged with N₂ for 3 times, and then the mixture was stirred at 80° C. for 12 hours under N₂ atmosphere. LC-MS showed 4, 7-diazaspiro [2.5]octane-4-carboxylate was consumed completely and desired mass was detected. The crude was added H₂O (30 mL), and extracted with EtOAc (90 mL*3). The combined organic layers were washed with brine (50 mL), dried over Na₂SO₄, filtered and concentrated under reduced pressure to give a residue. The residue was purified by prep-TLC (SiO₂, Petroleum ether/Ethyl acetate=5/1) give desired tert-butyl 7-(2, 4-difluorophenyl)-4, 7-diazaspiro [2.5]octane-4-carboxylate (110 mg, 339 μmol, 14.40% yield) as a yellow oil. MS (ESI): mass calcd. For C₁₇H₂₂F₂N₂O₂ 224.37, m/z found 325.1 [M+H]⁺.

Step 2: To a solution of tert-butyl 7-(2, 4-difluorophenyl)-4, 7-diazaspiro [2.5]octane-4-carboxylate (100 mg, 308 μmol, 1 eq) in HCl/EtOAc (4 M, 1.16 mL, 15 eq). The mixture was stirred at 15° C. for 1 hour. LC-MS showed tert-butyl 7-(2, 4-difluorophenyl)-4, 7-diazaspiro [2.5]octane-4-carboxylate was consumed completely and desired mass was detected. The reaction mixture was concentrated under reduced pressure to give desired 7-(2, 4-difluorophenyl)-4, 7-diazaspiro [2.5]octane (70 mg, crude) as a white solid. MS (ESI): mass calcd. For C₁₂H₁₄F₂N₂ 224.25, m/z found 225.0 [M+H]⁺.

Step 3: To a solution of (1E)-2,6-dichloro-N-(p-tolylsulfonyl)benzohydrazonoyl chloride (152 mg, 403 μmol, 1.5 eq) and 7-(2, 4-difluorophenyl)-4,7-diazaspiro [2.5]octane (70 mg, 269 μmol, 1 eq, HCl) in THF (5 mL) was added TEA (27.2 mg, 269 μmol, 37.4 μL, 1 eq). The mixture was stirred at 15° C. for 12 hours. LC-MS showed (1E)-2, 6-dichloro-N-(p-tolylsulfonyl) benzohydrazonoyl chloride was consumed completely and desired mass was detected. The crude was added H₂O (20 mL), and extracted with EtOAc (45 mL*3). The combined organic layers were washed with brine (10 mL), dried over Na₂SO₄, filtered and concentrated under reduced pressure to give desired N-[(E)-[(2,6-dichlorophenyl)-[7-(2,4-difluorophenyl)-4,7-diazaspiro[2.5]octan-4-yl]methylene]amino]-4-methyl-benzenesulfonamide (500 mg, crude) as a brown solid. MS (ESI): mass calcd. For C₂₆H₂₄Cl₂F₂N₄O₂S 565.46, m/z found 565.0 [M+H]⁺.

Step 4: To a solution of N-[(E)-[(2,6-dichlorophenyl)-[7-(2, 4-difluorophenyl)-4,7-diazaspiro[2.5]octan-4-yl]methylene]amino]-4-methyl-benzenesulfonamide (500 mg, 884 μmol, 1 eq) in DMF (10 mL) was added K₂CO₃ (611 mg, 4.42 mmol, 5 eq). The mixture was stirred at 100° C. for 12 hours. LC-MS showed N-[(E)-[(2,6-dichlorophenyl)-[7-(2, 4-difluorophenyl)-4, 7-diazaspiro [2.5]octan-4-yl]methylene]amino]-4-methyl-benzenesulfonamide was consumed completely and desired mass was detected. The reaction mixture was concentrated under reduced pressure to give a residue. The crude was added H₂O (20 mL), and extracted with EtOAc (15 mL*3). The combined organic layers were washed with brine (10 mL), dried over Na₂SO₄, filtered and concentrated under reduced pressure to give desired 4-chloro-3-[7-(2,4-difluorophenyl)-4,7-diazaspiro[2.5]octan-4-yl]-1-(p-tolylsulfonyl) indazole (470 mg, crude) as a brown oil. MS (ESI): mass calcd. For C₂₆H₂₃ClF₂N₄O₂S 529.00, m/z found 529.1 [M+H]⁺.

Step 5: To a solution of 4-chloro-3-[7-(2, 4-difluorophenyl)-4, 7-diazaspiro [2.5]octan-4-yl]-1-(p-tolylsulfonyl) indazole (470 mg, 888 μmol, 1 eq) in MeOH (10 mL) was added K₂CO₃ (1.23 g, 8.88 mmol, 10 eq). The mixture was stirred at 70° C. for 1 hour. LC-MS showed 4-chloro-3-[7-(2, 4-difluorophenyl)-4, 7-diazaspiro [2.5]octan-4-yl]-1-(p-tolylsulfonyl) indazole was consumed completely and desired mass was detected. The crude was added H₂O (20 mL), and extracted with EtOAc (15 mL*3). The combined organic layers were washed with brine (10 mL), dried over Na₂SO₄, filtered and concentrated under reduced pressure to give a residue. The residue was purified by prep-TLC (SiO₂, petroleum ether/ethyl acetate=2/1) give desired 4-chloro-3-[7-(2,4-difluorophenyl)-4,7-diazaspiro[2.5]octan-4-yl]-1H-indazole (70 mg, 187 μmol, 21.02% yield) as a yellow solid. MS (ESI): mass calcd. For C₁₉H₁₇ClF₂N₄ 374.82 m/z found 375.1 [M+H]⁺.

Step 6: To a solution of 4-chloro-3-[7-(2, 4-difluorophenyl)-4, 7-diazaspiro [2.5]octan-4-yl]-1H-indazole (70 mg, 187 μmol, 1 eq) and 4-(1, 1-difluoroethyl) benzenesulfonyl chloride (67.4 mg, 280 μmol, 1.5 eq) in DCM (1 mL) was added TEA (37.8 mg, 374 μmol, 52.0 μL, 2 eq) and DMAP (2.28 mg, 18.7 μmol, 0.1 eq). The mixture was stirred at 20° C. for 1 hour. LC-MS showed 4-chloro-3-[7-(2, 4-difluorophenyl)-4, 7-diazaspiro [2.5]octan-4-yl]-1H-indazole was consumed completely and desired mass was detected. The crude was added H₂O (20 mL), and extracted with DCM (15 mL*3). The combined organic layers were washed with brine (10 mL), dried over Na₂SO₄, filtered and concentrated under reduced pressure to give a residue. The residue was purified by prep-TLC (SiO₂, petroleum ether/ethyl acetate=2/1) give desired 4-chloro-1-[4-(1, 1-difluoroethyl) phenyl]sulfonyl-3-[7-(2, 4-difluorophenyl)-4, 7-diazaspiro [2.5]octan-4-yl]indazole (27.7 mg, 46.7 μmol, 25.0% yield, 97.68% purity) as a white solid. ¹H NMR (400 MHz, DMSO-d₆) δ 8.04 (d, J=8.3 Hz, 1H), 7.84 (d, J=8.3 Hz, 2H), 7.63-7.57 (m, 3H), 7.43 (d, J=7.7 Hz, 1H), 7.27-7.18 (m, 1H), 6.95 (ddd, J=3.0, 7.1, 14.3 Hz, 1H), 6.72-6.64 (m, 1H), 3.57 (br t, J=4.7 Hz, 2H), 3.30-3.23 (m, 2H), 2.99-2.76 (m, 2H), 1.87-1.75 (m, 1H), 1.81 (t, J=19.1 Hz, 2H), 0.79 (s, 2H), 0.42 (br s, 2H). HPLC: 97.68% (220 nm), 97.69% (215 nm), 98.43 (254 nm). MS (ESI): mass calcd. For C₂₇H₂₃ClF₄N₄O₂S 579.01 m/z found 579.1 [M+H]⁺.

### Compound 127: 4-(4-chloro-1-((4-(1, 1-difluoroethyl) phenyl) sulfonyl)-1H-indazol-3-yl)-4, 7-diazaspiro [2.5]octane-7-carbonyl fluoride

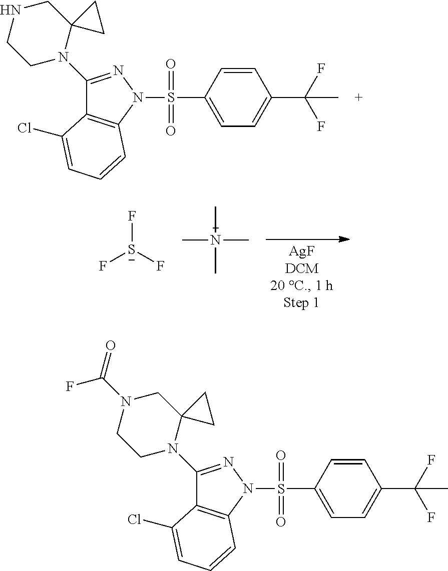

Step 1: To a solution of 4-chloro-3-(4, 7-diazaspiro [2.5]octan-4-yl)-1-[4-(1, 1-difluoroethyl) phenyl]sulfonyl-indazole (140 mg, 299 μmol, 1 eq) and tetramethylammonium; trifluoromethanethiolate (105 mg, 599 μmol, 2 eq) in DCM (3 mL) was added fluorosilver (190 mg, 1.50 mmol, 32.5 μL, 5 eq). The mixture was stirred at 15° C. for 1 hour. LCMS showed 4-chloro-3-(4, 7-diazaspiro [2.5]octan-4-yl)-1-[4-(1, 1-difluoroethyl) phenyl]sulfonyl-indazole was consumed completely and desired mass was detected. The reaction mixture was added to H₂O (30 mL), extracted with EtOAc (30 mL*3). The combined organic layers were washed with brine (20 mL) and dried over Na₂SO₄, filtered and concentrated under reduced pressure to give a residue. The residue was purified by prep-HPLC (TFA condition; column: Phenomenex Luna C18 75*30 mm*3 μm; mobile phase: [H₂O(0.1% TFA)-ACN]; gradient:40%-70% B over 8.0 min) to give desired 4-(4-chloro-1-((4-(1, 1-difluoroethyl)phenyl)sulfonyl)-1H-indazol-3-yl)-4,7-diazaspiro [2.5]octane-7-carbonyl fluoride (16 mg, 29.9 μmol, 9.98% yield, 100.00% purity) as a yellow solid. ¹H NMR (400 MHz, DMSO-d₆) δ 8.10-8.05 (m, 1H), 7.95-7.88 (m, 2H), 7.80-7.73 (m, 2H), 7.68-7.61 (m, 1H), 7.50-7.44 (m, 1H), 3.72-3.60 (m, 2H), 3.54-3.50 (m, 2H), 3.34-3.25 (m, 2H), 1.98-1.86 (m, 3H), 0.88-0.79 (m, 2H), 0.46-0.35 (m, 2H). HPLC: 100.00% (220 nm), 100.00% (215 nm), 100.00% (254 nm). MS (ESI): mass calcd. For C₂₂H₂₀ClF₃N₄O₃S 512.1, m/z found 513.0 [M+H]⁺.

### Compound 128: 4-chloro-1-((4-(1, 1-difluoroethyl) phenyl) sulfonyl)-3-(7-(1-fluoro-2-methylpropan-2-yl)-4, 7-diazaspiro [2.5]octan-4-yl)-1H-indazole

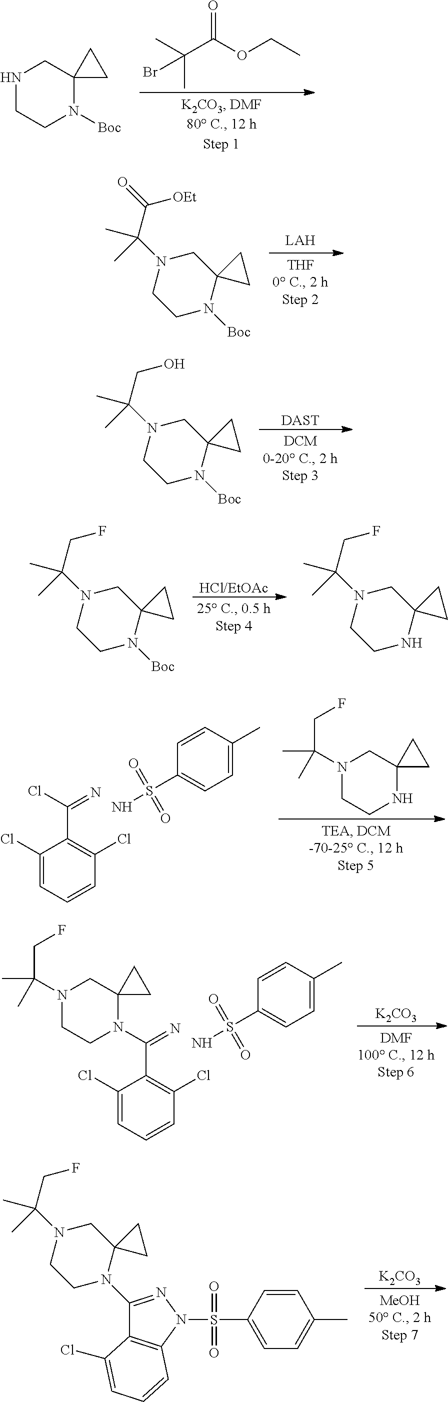

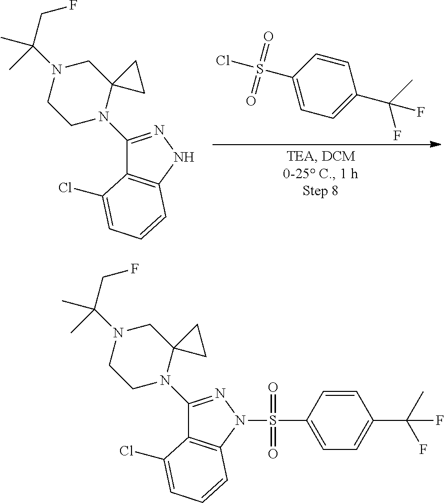

Step 1: To a solution of tert-butyl 4, 7-diazaspiro [2.5]octane-4-carboxylate (1 g, 4.71 mmol, 1 eq) and ethyl 2-bromo-2-methyl-propanoate (919 mg, 4.71 mmol, 691 μL, 1 eq) in DMF (30 mL) was added K₂CO₃ (1.95 g, 14.1 mmol, 3 eq). The mixture was stirred at 80° C. for 12 hours. LCMS showed tert-butyl 4, 7-diazaspiro [2.5]octane-4-carboxylate was consumed completely and desired mass was detected. The reaction mixture was concentrated to give desired tert-butyl 7-(1-ethoxy-2-methyl-1-oxopropan-2-yl)-4, 7-diazaspiro [2.5]octane-4-carboxylate (1.5 g, crude) as a yellow oil. MS (ESI): mass calcd. For C₁₇H₃₀N₂O₄ 326.22, m/z found 326.2 [M+H]⁺.

Step 2: To a solution of tert-butyl 7-(2-ethoxy-1,1-dimethyl-2-oxo-ethyl)-4,7-diazaspiro[2.5]octane-4-carboxylate (1.4 g, 4.29 mmol, 1 eq) in THF (20 mL) was added LAH (326 mg, 8.58 mmol, 2 eq) at 0° C. The mixture was stirred at 0° C. for 2 hours. LCMS showed tert-butyl 7-(1-ethoxy-2-methyl-1-oxopropan-2-yl)-4, 7-diazaspiro [2.5]octane-4-carboxylate was consumed completely and desired mass was detected. The reaction mixture was quenched by 10H₂O·Na₂SO₄ (1 g) at 0° C., and dried over Na₂SO₄, filtered and concentrated under reduced pressure to give a residue. The residue was purified by column chromatography (SiO₂, petroleum ether/ethyl acetate=50/1 to 0/1) to give desired tert-butyl 7-(1-hydroxy-2-methylpropan-2-yl)-4, 7-diazaspiro [2.5]octane-4-carboxylate (600 mg, 2.11 mmol, 49.2% yield) as a yellow oil. MS (ESI): mass calcd. For C₁₅H₂₈N₂O₃ 284.21, m/z found 285.2 [M+H]⁺.

Step 3: To a solution of tert-butyl 7-(2-hydroxy-1,1-dimethyl-ethyl)-4, 7-diazaspiro [2.5]octane-4-carboxylate (600 mg, 2.11 mmol, 1 eq) in DCM (200 mL) was added DAST (680 mg, 4.22 mmol, 557 μL, 2 eq) at 0° C. The mixture was stirred at 20° C. for 2 hours. LCMS showed tert-butyl 7-(1-hydroxy-2-methylpropan-2-yl)-4, 7-diazaspiro [2.5]octane-4-carboxylate was consumed completely and desired mass was detected. The reaction mixture was quenched by addition sat.aq.NaHCO₃ (10 mL) at 0° C., and extracted with EtOAc 30 mL (10 mL*3). The combined organic layers were dried over Na₂SO₄, filtered and concentrated under reduced pressure to give desired tert-butyl 7-(1-fluoro-2-methylpropan-2-yl)-4,7-diazaspiro[2.5]octane-4-carboxylate (600 mg, crude) as a white solid. MS (ESI): mass calcd. For C₁₅H₂₇FN₂O₂ 286.21, m/z found 287.2 [M+H]⁺.

Step 4: To a solution of tert-butyl 7-(2-fluoro-1, 1-dimethyl-ethyl)-4, 7-diazaspiro [2.5]octane-4-carboxylate (400 mg, 1.40 mmol, 1 eq) in HCl/EtOAc (4M, 10 mL). The mixture was stirred at 25° C. for 0.5 hour. LCMS showed tert-butyl 7-(1-fluoro-2-methylpropan-2-yl)-4, 7-diazaspiro [2.5]octane-4-carboxylate was consumed completely and desired mass was detected. The reaction mixture was concentrated under reduced pressure to give desired 7-(1-fluoro-2-methylpropan-2-yl)-4, 7-diazaspiro [2.5]octane (300 mg, crude) as a white solid. MS (ESI): mass calcd. For C₁₀H₁₉FN₂ 186.15, m/z found 187.2 [M+H]⁺.

Step 5: To a solution of 7-(2-fluoro-1, 1-dimethyl-ethyl)-4, 7-diazaspiro [2.5]octane (301 mg, 1.35 mmol, 1.02 eq, HCl) in DCM (30 mL) was added TEA (670 mg, 6.62 mmol, 921 μL, 5 eq) and (1E)-2,6-dichloro-N-(p-tolylsulfonyl)benzohydrazonoyl chloride (500 mg, 1.32 mmol, 1 eq) at −70° C. The mixture was stirred at 25° C. for 12 hours. LCMS showed (E)-2, 6-dichloro-N-tosylbenzohydrazonoyl chloride was consumed completely and desired mass was detected. The reaction mixture was concentrated under reduced pressure to give desired (E)-N′-((2, 6-dichlorophenyl) (7-(1-fluoro-2-methylpropan-2-yl)-4, 7-diazaspiro [2.5]octan-4-yl)methylene)-4-methylbenzenesulfonohydrazide (700 mg, crude) as a yellow oil. MS (ESI): mass calcd. For C₂₄H₂₉Cl₂FN₄O₂S 526.14, m/z found 527.1 [M+H]⁺.

Step 6: To a solution of N-[(E)-[(2, 6-dichlorophenyl)-[7-(2-fluoro-1, 1-dimethyl-ethyl)-4, 7-diazaspiro [2.5]octan-4-yl]methylene]amino]-4-methyl-benzenesulfonamide (700 mg, 1.33 mmol, 1 eq) in DMF (10 mL) was added K₂CO₃ (550 mg, 3.98 mmol, 3 eq). The mixture was stirred at 100° C. for 12 hours. LCMS showed (E)-N′-((2, 6-dichlorophenyl) (7-(1-fluoro-2-methylpropan-2-yl)-4, 7-diazaspiro [2.5]octan-4-yl) methylene)-4-methylbenzenesulfonohydrazide was consumed completely and desired mass was detected. The reaction mixture was filtered. The filtrate was concentrated under reduced pressure to give desired 4-chloro-3-(7-(1-fluoro-2-methylpropan-2-yl)-4, 7-diazaspiro [2.5]octan-4-yl)-1-tosyl-1H-indazole (650 mg, crude) as a yellow oil. MS (ESI): mass calcd. For C₂₄H₂₈ClFN₄O₂S 490.16, m/z found 491.2 [M+H]⁺.

Step 7: To a solution of 4-chloro-3-[7-(2-fluoro-1, 1-dimethyl-ethyl)-4, 7-diazaspiro [2.5]octan-4-yl]-1-(p-tolylsulfonyl) indazole (650 mg, 1.32 mmol, 1 eq) in MeOH (20 mL) was added K₂CO₃ (366 mg, 2.65 mmol, 2 eq). The mixture was stirred at 50° C. for 2 hours. LCMS showed 4-chloro-3-(7-(1-fluoro-2-methylpropan-2-yl)-4, 7-diazaspiro [2.5]octan-4-yl)-1-tosyl-1H-indazole was consumed completely and desired mass was detected. The reaction mixture was filtered. The filtrate was concentrated under reduced pressure to give a residue. The residue was purified by column chromatography (SiO₂, petroleum ether/ethyl acetate=50/1 to 1/1) to give desired 4-chloro-3-(7-(1-fluoro-2-methylpropan-2-yl)-4, 7-diazaspiro [2.5]octan-4-yl)-1H-indazole (300 mg, 891 μmol, 67.3% yield) as a yellow oil. MS (ESI): mass calcd. For C₁₇H₂₂ClFN₄ 336.15 m/z found 337.2 [M+H]⁺.

Step 8: To a solution of 4-chloro-3-[7-(2-fluoro-1,1-dimethyl-ethyl)-4,7-diazaspiro[2.5]octan-4-yl]-1H-indazole (150 mg, 445 μmol, 1 eq), 4-chloro-3-[7-(2-fluoro-1,1-dimethyl-ethyl)-4, 7-diazaspiro[2.5]octan-4-yl]-1H-indazole (150 mg, 445 μmol, 1 eq) and TEA (225 mg, 2.23 mmol, 310 μL, 5 eq) in DCM (2 mL) was added 4-(1,1-difluoroethyl)benzenesulfonyl chloride (161 mg, 668 μmol, 1.5 eq) and DMAP (5.44 mg, 44.5 μmol, 0.1 eq) at 0° C. The mixture was stirred at 25° C. for 1 hour. LCMS showed 4-chloro-3-(7-(1-fluoro-2-methylpropan-2-yl)-4, 7-diazaspiro [2.5]octan-4-yl)-1H-indazole was consumed completely and desired mass was detected. The reaction was concentrated to get a residue. The residue was purified by prep-HPLC (column: Waters Xbridge Prep OBD C18 150*40 mm*10 μm; mobile phase: [H₂O(10 mM NH₄HCO₃)-ACN]; gradient:65%-95% B over 8.0 min) to give desired 4-chloro-1-((4-(1, 1-difluoroethyl) phenyl) sulfonyl)-3-(7-(1-fluoro-2-methylpropan-2-yl)-4, 7-diazaspiro [2.5]octan-4-yl)-1H-indazole (31.6 mg, 56.8 μmol, 12.7% yield, 97.16% purity) as a white solid. ¹H NMR (400 MHz, DMSO-d₆) δ 8.07 (d, J=8.4 Hz, 1H), 7.88 (d, J=8.3 Hz, 2H), 7.77 (d, J=8.4 Hz, 2H), 7.62 (t, J=8.1 Hz, 1H), 7.44 (d, J=7.7 Hz, 1H), 3.44 (br t, J=4.5 Hz, 2H), 3.36-3.35 (m, 4H), 2.43 (s, 1H), 2.37 (s, 1H), 1.92 (t, J=19.1 Hz, 3H), 1.35 (s, 3H), 1.29 (s, 3H), 0.67 (br s, 2H), 0.36 (br s, 2H) HPLC: 97.16% (220 nm), 97.02% (215 nm), 97.75% (254 nm). MS (ESI): mass calcd. For C₂₅H₂₈ClF₃N₄O₂S 540.16 m/z found 541.2 [M+H]⁺.

### Compound 129: 4-chloro-1-((3-(1, 1-difluoroethyl) bicycle [1.1.1]pentan-1-yl) sulfonyl)-3-(7-(5-fluoropyrimidin-2-yl)-4, 7-diazaspiro [2.5]octan-4-yl)-1H-indazole

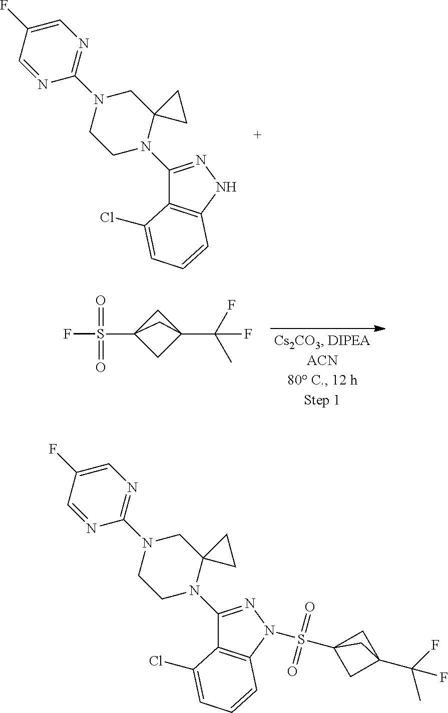 [SMILES: Fc1cnc(N2CCN(c3n[nH]c4cccc(Cl)c34)C3(CC3)C2)nc1 ; CC(F)(F)C12CC(S(=O)(=O)F)(C1)C2 ; CC(F)(F)C12CC(S(=O)(=O)n3nc(N4CCN(c5ncc(F)cn5)CC45CC5)c4c(Cl)cccc43)(C1)C2]

Step 1: The solution of 4-chloro-3-[7-(5-fluoropyrimidin-2-yl)-4, 7-diazaspiro [2.5]octan-4-yl]-1H-indazole (64.0 mg, 177 μmol, 2.0 eq), 3-(1, 1-difluoroethyl) bicycle [1.1.1]pentane-1-sulfonyl fluoride (19 mg, 88.7 μmol, 1.0 eq), Cs₂CO₃ (145 mg, 444 μmol, 5 eq), DIPEA (57.3 mg, 444 μmol, 77.3 μL, 5 eq) and bis [bis(trifluoromethylsulfonyl) amino] calcium (53.3 mg, 88.7 μmol, 1 eq) in ACN (5 mL) was stirred at 80° C. for 12 hours. LCMS showed 4-chloro-3-[7-(5-fluoropyrimidin-2-yl)-4, 7-diazaspiro [2.5]octan-4-yl]-1H-indazole remained and desired mass was detected. The reaction was filtered and the filtrate was concentrated to get a residue. The residue was purified by prep-HPLC (column: 3_Phenomenex Luna C18 75*30 mm*3 μm; mobile phase: [H₂O(0.1% TFA)-ACN]; gradient: 55%-100% B over 8.0 min) to give desired 4-chloro-1-[[3-(1, 1-difluoroethyl)-1-bicyclo [1.1.1]pentanyl]sulfonyl]-3-[7-(5-fluoropyrimidin-2-yl)-4, 7-diazaspiro [2.5]octan-4-yl]indazole (13.5 mg, 23.0 μmol, 25.95% yield, 94.29% purity) as a yellow solid. ¹H NMR (DMSO-d₆) δ 8.46 (br s, 2H), 7.77-8.05 (m, 1H), 7.36-7.69 (m, 2H), 3.97 (br s, 4H), 3.63 (br s, 2H), 2.12 (br s, 6H), 1.50 (br m, 3H), 0.82 (br s, 2H), 0.65 (br s, 2H) HPLC: 94.29% (220 nm), 95.41% (215 nm), 91.58% (254 nm). MS (ESI): mass calcd. For C₂₄H₂₄F₃SN₆ClO₂ 552.13 m/z found 553.0 [M+H]⁺.

### Compound 130: 4-chloro-1-[4-(1, 1-difluoroethyl) phenyl]sulfonyl-3-[7-[2-fluoro-1-(fluoromethyl)-1-methyl-ethyl]-4, 7-diazaspiro [2.5]octan-4-yl]indazole

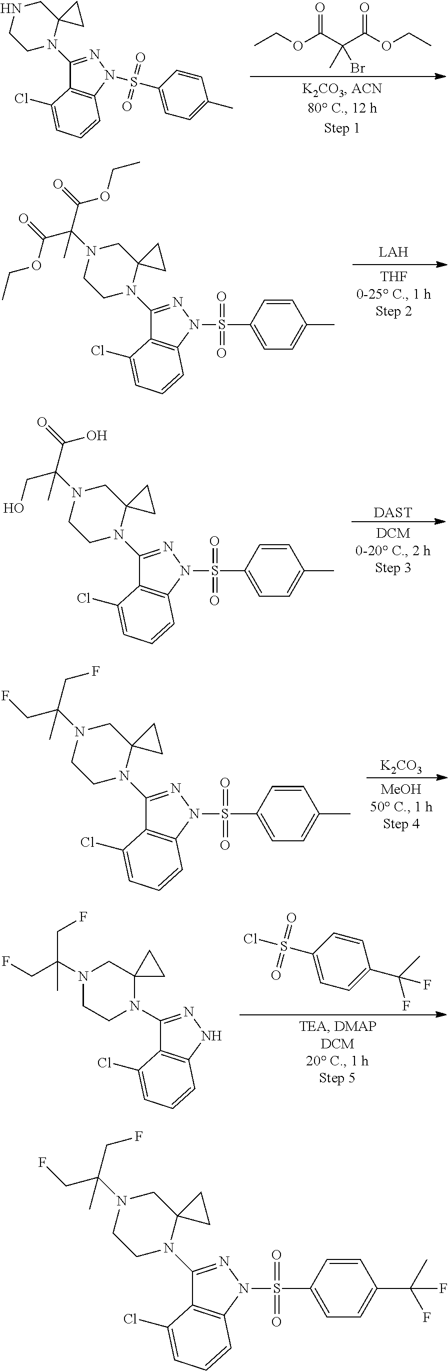

Step 1: To the solution of 4-chloro-3-(4, 7-diazaspiro [2.5]octan-4-yl)-1-(p-tolylsulfonyl) indazole (1 g, 2.40 mmol, 1 eq) in ACN (30 mL) was added K₂CO₃ (663 mg, 4.80 mmol, 2 eq). To the reaction mixture was added diethyl 2-bromo-2-methyl-propanedioate (911 mg, 3.60 mmol, 1.5 eq) and the solution was stirred at 80° C. for 12 hours. LCMS showed diethyl 2-[4-[4-chloro-1-(p-tolylsulfonyl) indazol-3-yl]-4, 7-diazaspiro [2.5]octan-7-yl]-2-methyl-propanedioate was consumed completely and desired mass was detected. The reaction was concentrated to get a residue. The residue was purified by column chromatography (SiO₂, petroleum ether/ethyl acetate=20/1 to 10/1) to give desired diethyl 2-[4-[4-chloro-1-(p-tolylsulfonyl) indazol-3-yl]-4, 7-diazaspiro [2.5]octan-7-yl]-2-methyl-propanedioate (800 mg, crude) as a yellow solid. MS (ESI): mass calcd. For C₂₈H₃₃ClN₄O₆S 588.18 m/z found 589.2 [M+H]⁺.

Step 2: The mixture of diethyl 2-[4-[4-chloro-1-(p-tolylsulfonyl) indazol-3-yl]-4, 7-diazaspiro [2.5]octan-7-yl]-2-methyl-propanedioate (500 mg, 679 μmol, 1 eq) in THF (15 mL) was added LiAlH₄ (51.5 mg, 1.36 mmol, 2 eq) at 0° C. for 0.5 hour, then the reaction was stirred at 25° C. for 0.5 hour. LCMS showed diethyl 2-[4-[4-chloro-1-(p-tolylsulfonyl) indazol-3-yl]-4, 7-diazaspiro [2.5]octan-7-yl]-2-methyl-propanedioate was consumed completely and desired mass was detected. The mixture was dried over Na₂SO₄·10H₂O, filtered and concentrated under reduced pressure to give a residue. The residue was purified by column chromatography (SiO₂, petroleum ether/ethyl acetate=20/1 to 10/1) to give desired 2-chloro-N-(3, 3-difluorocyclobutyl)-N-methyl-6-nitro-aniline (300 mg, crude) as a yellow solid. MS (ESI): mass calcd. For C₂₄H₂₉ClN₄O₄S 504.16 m/z found 505.2 [M+H]⁺.

Step 3: The mixture of 2-[4-[4-chloro-1-(p-tolylsulfonyl) indazol-3-yl]-4, 7-diazaspiro [2.5]octan-7-yl]-2-methyl-propane-1,3-diol (300 mg, 594 μmol, 1 eq) in DCM (6 mL) was added DAST (383 mg, 2.38 mmol, 4 eq) at 0° C., the reaction was stirred at 25° C. for 1 hour. LCMS showed 2-[4-[4-chloro-1-(p-tolylsulfonyl) indazol-3-yl]-4, 7-diazaspiro [2.5]octan-7-yl]-2-methyl-propane-1, 3-diol was consumed completely and desired mass was detected. The reaction mixture was quenched by addition saturated aqueous NaHCO₃ solution (20 mL) at 0° C., and then extracted with DCM (20 mL*3). The combined organic layers were dried over Na₂SO₄, filtered and concentrated under reduced pressure to give a residue. The residue was purified by column chromatography (SiO₂, petroleum ether/Ethyl acetate=5/1 to 3/1) to give desired 4-chloro-3-[7-[2-fluoro-1-(fluoromethyl)-1-methyl-ethyl]-4, 7-diazaspiro [2.5]octan-4-yl]-1-(p-tolylsulfonyl) indazole (200 mg, crude) as a yellow solid. MS (ESI): mass calcd. For C₂₄H₂₇ClF₂N₄O₂S 508.15 m/z found 509.2 [M+H]⁺.

Step 4: The mixture of 4-chloro-3-[7-[2-fluoro-1-(fluoromethyl)-1-methyl-ethyl]-4, 7-diazaspiro [2.5]octan-4-yl]-1-(p-tolylsulfonyl) indazole (100 mg, 196 μmol, 1 eq) in MeOH (3 mL) was added K₂CO₃ (54.3 mg, 393 μmol, 2 eq), the reaction was stirred at 50° C. for 1 hour. LCMS showed 4-chloro-3-[7-[2-fluoro-1-(fluoromethyl)-1-methyl-ethyl]-4, 7-diazaspiro [2.5]octan-4-yl]-1-(p-tolylsulfonyl) indazole was consumed completely and desired mass was detected. The reaction mixture was quenched by addition water (10 mL) at 0° C., and then extracted with Ethyl acetate (10 mL*3). The combined organic layers were dried over Na₂SO₄, filtered and concentrated under reduced pressure to give a residue. The residue was purified by column chromatography (SiO₂, petroleum ether/ethyl acetate=5/1 to 3/1) to give desired 4-chloro-3-[7-[2-fluoro-1-(fluoromethyl)-1-methyl-ethyl]-4, 7-diazaspiro [2.5]octan-4-yl]-1H-indazole (80 mg, crude) as a yellow solid. MS (ESI): mass calcd. For C₁₇H₂₁ClF₂N₄ 354.14 m/z found 355.2 [M+H]⁺.

Step 5: The mixture of 4-chloro-3-[7-[2-fluoro-1-(fluoromethyl)-1-methyl-ethyl]-4, 7-diazaspiro [2.5]octan-4-yl]-1H-indazole (80 mg, 141 μmol, 1 eq), TEA (71.3 mg, 705 μmol, 5 eq), DMAP (3.44 mg, 28.2 μmol, 0.2 eq) and 4-(1, 1-difluoroethyl)benzenesulfonyl chloride (67.8 mg, 282 μmol, 2 eq) in DCM (3 mL) was stirred at 20° C. for 1 hour. LCMS showed 4-chloro-3-[7-[2-fluoro-1-(fluoromethyl)-1-methyl-ethyl]-4, 7-diazaspiro [2.5]octan-4-yl]-1H-indazole was consumed completely and desired mass was detected. The reaction was concentrated to get a residue. The residue was purified by prep-TLC (SiO₂, petroleum ether/ethyl acetate=5/1) to give desired 4-chloro-1-[4-(1, 1-difluoroethyl) phenyl]sulfonyl-3-[7-[2-fluoro-1-(fluoromethyl)-1-methyl-ethyl]-4, 7-diazaspiro [2.5]octan-4-yl]indazole (19.8 mg, 35.1 μmol, 24.9% yield, 99.05% purity) as a white solid. ¹H NMR (400 MHz, DMSO-d₆) δ 8.07 (d, J=8.4 Hz, 1H), 7.89 (d, J=8.4 Hz, 2H), 7.77 (d, J=8.5 Hz, 2H), 7.63 (t, J=8.1 Hz, 1H), 7.45 (d, J=7.8 Hz, 1H), 4.73-4.31 (m, 2H), 3.44 (br t, J=4.2 Hz, 2H), 2.58 (s, 1H), 2.54-2.53 (m, 1H), 2.19 (s, 1H), 1.93 (t, J=19.1 Hz, 3H), 1.38-1.29 (m, 6H), 0.69 (br s, 2H), 0.36 (br s, 2H). HPLC: 95.05% (220 nm), 94.67% (215 nm), 99.05% (254 nm). MS (ESI): mass calcd. For C₂₅H₂₇ClF₄N₄O₂S 558.15 m/z found 559.2 [M+H]⁺.

### Compound 131: 4-chloro-3-[7-(5-fluoropyrimidin-2-yl)-4, 7-diazaspiro [2.5]octan-4-yl]-1-[[3-(trifluoromethyl)-1-bicyclo [1.1.1]pentanyl]sulfonyl]indazole

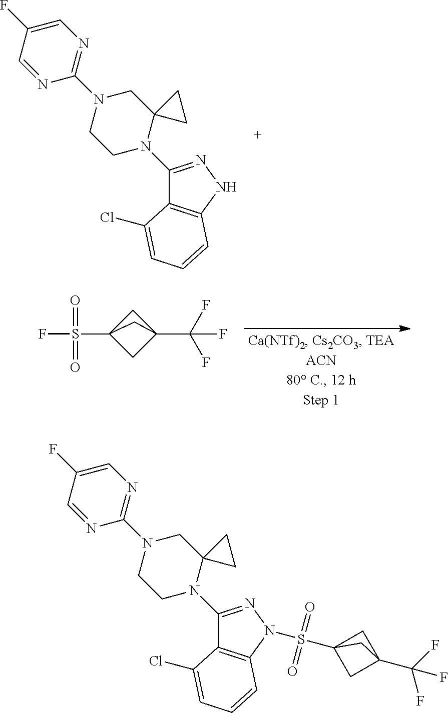

Step 1: To a solution of 3-(trifluoromethyl)bicyclo[1.1.1]pentane-1-sulfonyl fluoride (9.12 mg, 41.8 μmol, 1.5 eq), 4-chloro-3-[7-(5-fluoropyrimidin-2-yl)-4,7-diazaspiro[2.5]octan-4-yl]-1H-indazole (10 mg, 27.9 μmol, 1 eq) in ACN (1.5 mL) was added bis[bis(trifluoromethylsulfonyl)amino]calcium (20.1 mg, 33.4 μmol, 1.2 eq) and Cs₂CO₃ (45.4 mg, 139 μmol, 5 eq), TEA (14.1 mg, 139 μmol, 19.4 μL, 5 eq). The mixture was stirred at 80° C. for 12 hours. LCMS showed 4-chloro-3-[7-(5-fluoropyrimidin-2-yl)-4, 7-diazaspiro [2.5]octan-4-yl]-1H-indazole was consumed completely and desired mass was detected. The reaction mixture was concentrated to give desired 4-chloro-3-[7-(5-fluoropyrimidin-2-yl)-4, 7-diazaspiro [2.5]octan-4-yl]-1-[[3-(trifluoromethyl)-1-bicyclo [1.1.1]pentanyl]sulfonyl]indazole (6.2 mg, 11.1 μmol, 39.9% yield, 98.05% purity) as a white solid. ¹H NMR (400 MHz, DMSO-d₆) δ 8.46 (s, 2H), 7.86 (d, J=8.1 Hz, 1H), 7.59 (t, J=8.1 Hz, 1H), 7.48 (d, J=7.4 Hz, 1H), 3.96 (s, 2H), 3.78 (br s, 2H), 3.63 (br d, J=4.8 Hz, 2H), 2.35 (s, 6H), 0.83-0.78 (m, 2H), 0.66 (s, 2H). HPLC: 98.05% (220 nm), 98.26% (215 nm), 100.00% (254 nm). MS (ESI): mass calcd. For C₂₃H₂₁ClF₄N₆O₂S 556.11 m/z found 557.1 [M+H]⁺.

### Compound 132: 4-chloro-1-[(3-fluoro-1-bicyclo [1.1.1]pentanyl) sulfonyl]-3-[7-(5-fluoropyrimidin-2-yl)-4, 7-diazaspiro [2.5]octan-4-yl]indazole

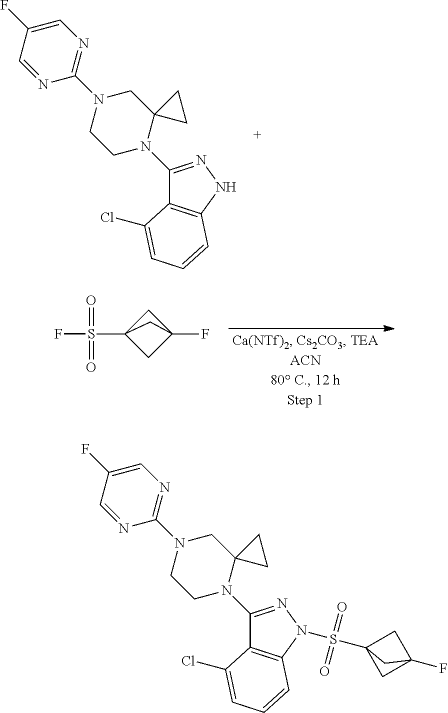 [SMILES: Fc1cnc(N2CCN(c3n[nH]c4cccc(Cl)c34)C3(CC3)C2)nc1 ; O=S(=O)(F)C12CC(F)(C1)C2 ; O=S(=O)(n1nc(N2CCN(c3ncc(F)cn3)CC23CC3)c2c(Cl)cccc21)C12CC(F)(C1)C2]

Step 1: To a solution of 3-fluorobicyclo[1.1.1]pentane-1-sulfonyl fluoride (7.03 mg, 41.8 μmol, 1.5 eq) in ACN (1.5 mL) was added 4-chloro-3-[7-(5-fluoropyrimidin-2-yl)-4, 7-diazaspiro [2.5]octan-4-yl]-1H-indazole (10 mg, 27.9 μmol, 1 eq), bis [bis(trifluoromethylsulfonyl) amino] calcium (20.1 mg, 33.4 μmol, 1.2 eq) and Cs₂CO₃ (45.4 mg, 139 μmol, 5 eq), TEA (14.1 mg, 139 μmol, 19.4 μL, 5 eq). The mixture was stirred at 80° C. for 12 hours. LCMS showed 4-chloro-3-[7-(5-fluoropyrimidin-2-yl)-4, 7-diazaspiro [2.5]octan-4-yl]-1H-indazole was consumed completely and desired mass was detected. The reaction mixture was concentrated to give desired 4-chloro-1-[(3-fluoro-1-bicyclo [1.1.1]pentanyl) sulfonyl]-3-[7-(5-fluoropyrimidin-2-yl)-4, 7-diazaspiro [2.5]octan-4-yl]indazole (7.2 mg, 14.2 μmol, 51.0% yield, 98.40% purity) as a white solid. ¹H NMR (400 MHz, DMSO-d₆) δ 8.48-8.44 (m, 2H), 7.86 (d, J=8.5 Hz, 1H), 7.60 (t, J=8.1 Hz, 1H), 7.48 (d, J=7.6 Hz, 1H), 3.96 (s, 2H), 3.79 (br s, 2H), 3.66-3.58 (m, 2H), 2.45 (d, J=2.0 Hz, 6H), 0.84-0.79 (m, 2H), 0.66 (s, 2H). HPLC: 98.40% (220 nm), 98.80% (215 nm), 98.58% (254 nm). MS (ESI): mass calcd. For C₂₂H₂₁ClF₂N₆O₂S 506.11 m/z found 507.1 [M+H]⁺.

### Compound 133: 1-[4-(1, 1-difluoroethyl) phenyl]sulfonyl-4-fluoro-3-[7-(5-fluoropyrimidin-2-yl)-4, 7-diazaspiro [2.5]octan-4-yl]indazole

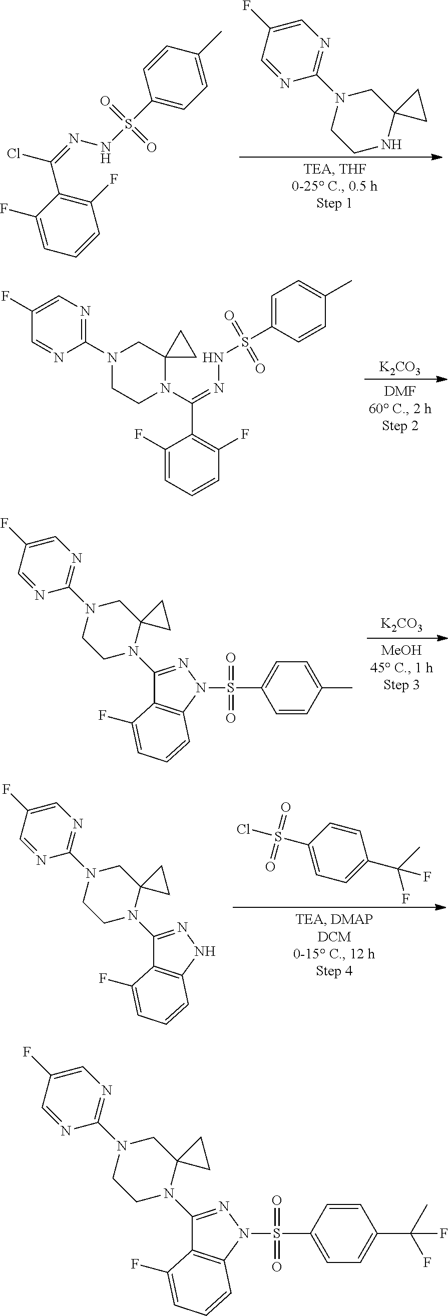

Step 1: To a solution of 7-(5-fluoropyrimidin-2-yl)-4, 7-diazaspiro [2.5]octane (300 mg, 1.44 mmol, 1 eq) in THF (10 mL) was added dropwise TEA (1.46 g, 14.4 mmol, 2.0 mL, 10 eq) at 25° C. After addition, the mixture was stirred at this temperature for 10 mins, and then (1Z)-2, 6-difluoro-N-(p-tolylsulfonyl) benzohydrazonoyl chloride (745 mg, 2.16 mmol, 1.5 eq) in THF (3 mL) was added dropwise at 0° C. The resulting mixture was stirred at 25° C. for 20 mins. LC-MS showed 7-(5-fluoropyrimidin-2-yl)-4, 7-diazaspiro [2.5]octane was consumed completely and one main peak with desired mass was detected. Then it was separated between 20 mL of H₂O and 40 mL of EtOAc. The organic phase was separated, washed with 30 mL of brine, dried over Na₂SO₄, filtered and concentrated under reduced pressure to give desired N—[(Z)-[(2,6-difluorophenyl)-[7-(5-fluoropyrimidin-2-yl)-4,7-diazaspiro[2.5]octan-4-yl]methylene]amino]-4-methyl-benzenesulfonamide (740 mg, crude) as a yellow solid. MS (ESI): mass calcd. For C₂₄H₂₃F₃N₆SO₂ 516.16 m/z found 517.2 [M+H]+

Step 2: To a solution of N—[(Z)-[(2, 6-difluorophenyl)-[7-(5-fluoropyrimidin-2-yl)-4, 7-diazaspiro [2.5]octan-4-yl]methylene]amino]-4-methyl-benzenesulfonamide (740 mg, 1.43 mmol, 1 eq) in DMF (10 mL) was added K₂CO₃ (990 mg, 7.16 mmol, 5 eq). The mixture was stirred at 60° C. for 2 hours. LC-MS showed N—[(Z)-[(2, 6-difluorophenyl)-[7-(5-fluoropyrimidin-2-yl)-4, 7-diazaspiro [2.5]octan-4-yl]methylene]amino]-4-methyl-benzenesulfonamide was consumed completely and desired mass was detected. The reaction mixture was added to H₂O (30 mL), extracted with EtOAc (30 mL*3). The combined organic layers were washed with brine 20 mL and dried over Na₂SO₄, filtered and concentrated under reduced pressure to give desired 4-fluoro-3-[7-(5-fluoropyrimidin-2-yl)-4, 7-diazaspiro [2.5]octan-4-yl]-1-(p-tolylsulfonyl) indazole (480 mg, crude) as a yellow oil. MS (ESI): mass calcd. For C₂₄H₂F₂N₆SO₂ 496.15 m/z found 497.2 [M+H]+

Step 3: To a solution of 4-fluoro-3-[7-(5-fluoropyrimidin-2-yl)-4, 7-diazaspiro [2.5]octan-4-yl]-1-(p-tolylsulfonyl) indazole (480 mg, 966 μmol, 1 eq) in MeOH (10 mL) was added K₂CO₃ (668 mg, 4.83 mmol, 5 eq). The mixture was stirred at 45° C. for 1 hour. LC-MS showed 4-fluoro-3-[7-(5-fluoropyrimidin-2-yl)-4, 7-diazaspiro [2.5]octan-4-yl]-1-(p-tolylsulfonyl) indazole was consumed completely and desired mass was detected. The reaction mixture was diluted with H₂O (30 mL), extracted with EtOAc (50 mL*3). The combined organic phase was washed with brine 150 mL (50 mL*3), dried over Na₂SO₄, filtered and concentrated in vacuum to give a residue. The residue was purified by prep-TLC (SiO₂, petroleum ether/ethyl acetate=3/1) to give desired 4-fluoro-3-[7-(5-fluoropyrimidin-2-yl)-4, 7-diazaspiro [2.5]octan-4-yl]-1H-indazole (220 mg, 642 μmol, 66.5% yield) as a light yellow solid. MS (ESI): mass calcd. For C₁₇H₁₆F₂N₆ 342.14 m/z found 343.1 [M+H]⁺.

Step 4: To a solution of 4-fluoro-3-[7-(5-fluoropyrimidin-2-yl)-4, 7-diazaspiro [2.5]octan-4-yl]-1H-indazole (50 mg, 146 μmol, 1 eq) and 4-(1, 1-difluoroethyl) benzenesulfonyl chloride (52.7 mg, 219 μmol, 1.5 eq) in DCM (2 mL) was added TEA (29.6 mg, 292 μmol, 40.7 μL, 2 eq) and DMAP (1.78 mg, 14.6 μmol, 0.1 eq) at 0° C. The mixture was stirred at 15° C. for 12 hours. LC-MS showed 4-fluoro-3-[7-(5-fluoropyrimidin-2-yl)-4, 7-diazaspiro [2.5]octan-4-yl]-1H-indazole was consumed completely and desired mass was detected. The reaction mixture was added to H₂O (30 mL), extracted with EtOAc (30 mL*3). The combined organic layers were washed with brine (20 mL) and dried over Na₂SO₄, filtered and concentrated under reduced pressure to give a residue. The residue was purified by prep-HPLC (TFA condition; Method:column: Phenomenex Luna C18 75*30 mm*3 μm; mobile phase: [H₂O(0.1% TFA)-ACN]; gradient:50%-80% B over 8.0 min) to give desired 1-[4-(1, 1-difluoroethyl) phenyl]sulfonyl-4-fluoro-3-[7-(5-fluoropyrimidin-2-yl)-4, 7-diazaspiro [2.5]octan-4-yl]indazole (25 mg, 45.7 μmol, 12.5% yield, 99.75% purity) as a yellow solid. ¹H NMR (400 MHz, DMSO-d₆) δ 8.46 (s, 2H), 7.96-7.85 (m, 3H), 7.72-7.66 (m, 3H), 7.22 (dd, J=8.1, 10.7 Hz, 1H), 3.88 (s, 2H), 3.61 (br d, J=4.5 Hz, 2H), 3.57-3.39 (m, 2H), 1.87 (t, J=19.1 Hz, 3H), 0.94-0.72 (m, 2H), 0.62-0.43 (m, 2H). HPLC: 99.35% (220 nm), 99.27% (215 nm), 99.75% (254 nm). MS (ESI): mass calcd. For C₂₅H₂₂N₆F₄O₂S 546.15 m/z found 547.2 [M+H]⁺.

### Compound 134: 1-[4-(1, 1-difluoroethyl) phenyl]sulfonyl-3-[7-(5-fluoropyrimidin-2-yl)-4, 7-diazaspiro [2.5]octan-4-yl]indazol-4-ol

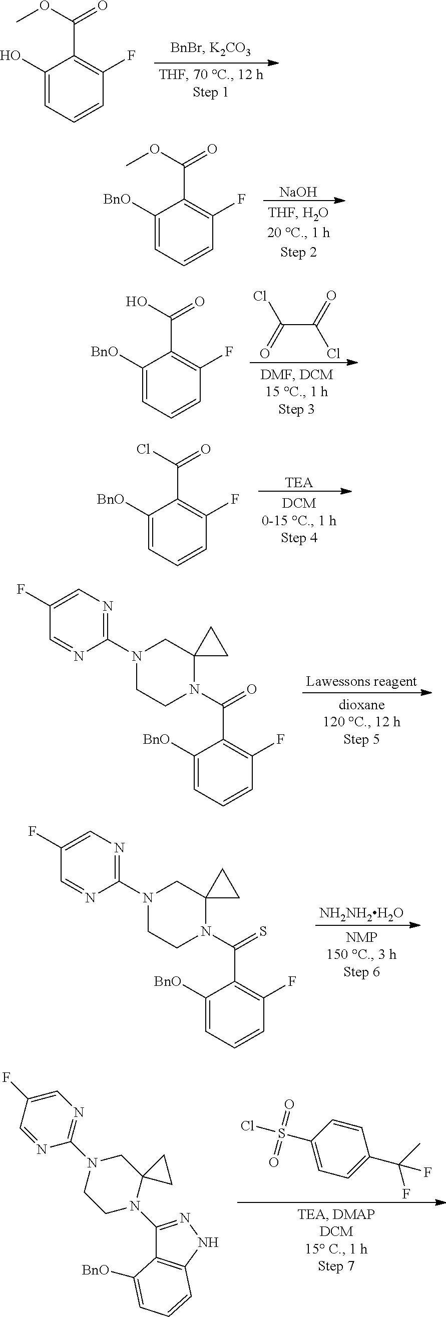

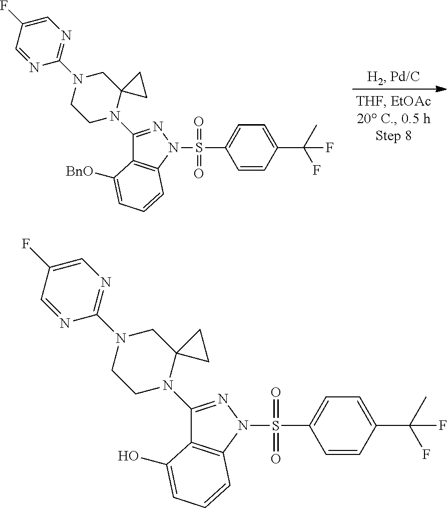 [SMILES: CC(F)(F)c1ccc(S(=O)(=O)n2nc(N3CCN(c4ncc(F)cn4)CC34CC4)c3c(OCc4ccccc4)cccc32)cc1 ; "H2, Pd/C" ; CC(F)(F)c1ccc(S(=O)(=O)n2nc(N3CCN(c4ncc(F)cn4)CC34CC4)c3c(O)cccc32)cc1]

Step 1: The mixture of methyl 2-fluoro-6-hydroxy-benzoate (1.36 g, 7.99 mmol, 1 eq), bromomethylbenzene (1.50 g, 8.79 mmol, 1.04 mL, 1.1 eq) and K₂CO₃ (3.98 g, 28.8 mmol, 3.6 eq) in THF (30 mL) was stirred at 70° C. for 12 hours. TLC (petroleum ether/EtOAc=3/1) showed methyl 2-fluoro-6-hydroxy-benzoate was consumed completely and one new spot formed. The reaction was filtered and the filtrate was concentrated to get a residue. The residue was purified by flash silica gel chromatography (ISCO®; 40 g SepaFlash® Silica Flash Column, Eluent of 0˜10% ethyl acetate/petroleum ether gradient @80 mL/min) to give desired methyl 2-benzyloxy-6-fluoro-benzoate (1.9 g, crude) as a white solid.

Step 2: To a solution of methyl 2-benzyloxy-6-fluoro-benzoate (500 mg, 1.92 mmol, 1 eq) in THF (5 mL) and H₂O (1 mL) was added NaOH (154 mg, 3.84 mmol, 2 eq). The mixture was stirred at 20° C. for 1 hour. TLC (petroleum ether/EtOAc=3/1) indicated methyl 2-benzyloxy-6-fluoro-benzoate was consumed completely and one new spot formed. The reaction was clean according to TLC (petroleum ether/EtOAc=3/1). The mixture to pH 2 with 1M HCl. Add EtOAc to the suspension and rinse with water. The organic layer is dried on Na₂SO₄, filtered, and the solvent removed in a vacuum to give desired 2-benzyloxy-6-fluoro-benzoic acid (330 mg, crude) as a white oil.

Step 3: To a solution of 2-benzyloxy-6-fluoro-benzoic acid (330 mg, 1.34 mmol, 1 eq) and (COCl)₂ (204 mg, 1.61 mmol, 141 μL, 1.2 eq) in DCM (5 mL) was added DMF (9.8 mg, 134 μmol, 10.3 μL, 0.1 eq). The mixture was stirred at 15° C. for 1 hour. TLC (petroleum ether/EtOAc=3/1) indicated 2-benzyloxy-6-fluoro-benzoic acid was consumed completely and one new spot formed. The reaction was clean according to TLC (petroleum ether/EtOAc=3/1). The reaction mixture was partitioned between H₂O (30 mL) and DCM (50 mL). The organic phase was separated, washed with brine (50 mL), dried over Na₂SO₄, filtered and concentrated under reduced pressure to give desired 2-benzyloxy-6-fluoro-benzoyl chloride (350 mg, crude) as a white oil.

Step 4: To a solution of 2-benzyloxy-6-fluoro-benzoyl chloride (350 mg, 1.32 mmol, 1 eq) and 7-(5-fluoropyrimidin-2-yl)-4,7-diazaspiro[2.5]octane (275 mg, 1.32 mmol, 1 eq) in DCM (5 mL) was added TEA (2.68 g, 26.5 mmol, 3.68 mL, 20 eq) at 0° C. The mixture was stirred at 15° C. for 1 hour. LC-MS showed 2-benzyloxy-6-fluoro-benzoyl chloride was consumed completely and desired mass was detected. The reaction mixture was partitioned between H₂O (10 mL) and DCM (10 mL). The organic phase was separated, washed with brine (10 mL), dried over Na₂SO₄, filtered and concentrated under reduced pressure to give desired (2-benzyloxy-6-fluoro-phenyl)-[7-(5-fluoropyrimidin-2-yl)-4,7-diazaspiro[2.5]octan-4-yl]methanone (550 mg, crude) as a yellow solid. MS (ESI): mass calcd. For C₂₄H₂₂F₂N₄O₂ 436.17, m/z found 437.2 [M+H]⁺.

Step 5: To a solution of (2-benzyloxy-6-fluoro-phenyl)-[7-(5-fluoropyrimidin-2-yl)-4, 7-diazaspiro [2.5]octan-4-yl]methanone (400 mg, 916 μmol, 1 eq) in dioxane (4 mL) was added 2, 4-bis(4-methoxyphenyl)-2, 4-dithioxo-1, 3, 2, 4-dithiadiphosphetane (741 mg, 1.83 mmol, 2 eq). The mixture was stirred at 120° C. for 12 hours. LC-MS showed (2-benzyloxy-6-fluoro-phenyl)-[7-(5-fluoropyrimidin-2-yl)-4, 7-diazaspiro [2.5]octan-4-yl]methanone was consumed completely and one main peak with desired mass was detected. The reaction mixture was partitioned between H₂O (10 mL) and EtOAc (10 mL). The organic phase was separated, washed with brine (50 mL), dried over Na₂SO₄, filtered and concentrated under reduced pressure to give the crude product. The residue was purified by prep-TLC (SiO₂, petroleum ether/EtOAc=4/1) to give desired (2-benzyloxy-6-fluoro-phenyl)-[7-(5-fluoropyrimidin-2-yl)-4, 7-diazaspiro [2.5]octan-4-yl]methanethione (40 mg, 88.4 μmol, 9.64% yield) as a white solid. MS (ESI): mass calcd. For C₂₄H₂₂F₂N₄OS 452.15, m/z found 453.2 [M+H]⁺.

Step 6: To a solution of (2-benzyloxy-6-fluoro-phenyl)-[7-(5-fluoropyrimidin-2-yl)-4, 7-diazaspiro [2.5]octan-4-yl]methanethione (100 mg, 221 μmol, 1 eq) in NMP (0.5 mL) was added NH₂NH₂·H₂O (0.5 mL). The mixture was stirred at 150° C. for 3 hours. LC-MS showed (2-benzyloxy-6-fluoro-phenyl)-[7-(5-fluoropyrimidin-2-yl)-4, 7-diazaspiro [2.5]octan-4-yl]methanethione remained and one main peak with desired mass was detected. Three reactions were combined for workup. The reaction mixture was partitioned between H₂O (30 mL) and EtOAc (30 mL). The organic phase was separated, washed with brine (50 mL), dried over Na₂SO₄, filtered and concentrated under reduced pressure to give desired 4-benzyloxy-3-[7-(5-fluoropyrimidin-2-yl)-4, 7-diazaspiro [2.5]octan-4-yl]-1H-indazole (530 mg, crude) as a yellow oil. MS (ESI): mass calcd. For C₂₄H₂₃FN₆O 430.19, m/z found 431.2 [M+H]⁺.

Step 7: To a solution of 4-benzyloxy-3-[7-(5-fluoropyrimidin-2-yl)-4, 7-diazaspiro [2.5]octan-4-yl]-1H-indazole (60 mg, 139 μmol, 1 eq) and 4-(1, 1-difluoroethyl) benzenesulfonyl chloride (50.3 mg, 209 μmol, 1.5 eq) 4-benzyloxy-3-[7-(5-fluoropyrimidin-2-yl)-4, 7-diazaspiro [2.5]octan-4-yl]-1H-indazole (60 mg, 139 μmol, 1 eq) in DCM (1 mL) was added TEA (28.2 mg, 279 μmol, 38.8 μL, 2 eq) and DMAP (1.70 mg, 13.9 μmol, 0.1 eq). The mixture was stirred at 15° C. for 1 hour. LC-MS showed 4-benzyloxy-3-[7-(5-fluoropyrimidin-2-yl)-4, 7-diazaspiro [2.5]octan-4-yl]-1H-indazole remained and one main peak with desired mass was detected. The reaction mixture was added to water (15 mL), extracted with DCM (30 mL). The combined organic layers were washed with brine (15 mL) and dried over Na₂SO₄, filtered and concentrated under reduced pressure to give a residue. The residue was purified by prep-TLC (SiO₂, petroleum ether/EtOAc=3/1) to give desired 4-benzyloxy-1-[4-(1,1-difluoroethyl)phenyl]sulfonyl-3-[7-(5-fluoropyrimidin-2-yl)-4, 7-diazaspiro[2.5]octan-4-yl]indazole (80 mg, 126 μmol, 90.4% yield) as a yellow oil. MS (ESI): mass calcd. For C₃₂H₂₉F₃N₆O₃S 634.20, m/z found 635.2 [M+H]⁺.

Step 8: To a solution of 4-benzyloxy-1-[4-(1,1-difluoroethyl)phenyl]sulfonyl-3-[7-(5-fluoropyrimidin-2-yl)-4,7-diazaspiro[2.5]octan-4-yl]indazole (25 mg, 39.4 μmol, 1 eq) in THF (1 mL) and EtOAc (2 mL) was added Pd/C (41.9 mg, 39.4 μmol, 10% purity, 1 eq). The suspension was degassed and purged with H₂ (15 psi) for 3 times. The mixture was stirred under H₂ at 20° C. for 0.5 hour. LC-MS showed 4-benzyloxy-1-[4-(1, 1-difluoroethyl)phenyl]sulfonyl-3-[7-(5-fluoropyrimidin-2-yl)-4, 7-diazaspiro[2.5]octan-4-yl]indazole remained and one main peak with desired mass was detected. The reaction mixture was added to water (15 mL), extracted with EtOAc (30 mL). The combined organic layers were washed with brine (15 mL) and dried over Na₂SO₄, filtered and concentrated under reduced pressure to give a residue. The residue was purified by prep-HPLC (TFA condition; column: Phenomenex Luna C18 75*30 mm*3 μm; mobile phase: [H₂O (0.1% TFA)-ACN]; gradient: 45%-75% B over 8.0 mins) to give desired 1-[4-(1, 1-difluoroethyl) phenyl]sulfonyl-3-[7-(5-fluoropyrimidin-2-yl)-4, 7-diazaspiro [2.5]octan-4-yl]indazol-4-ol (7.8 mg, 14.3 μmol, 12.1% yield, 98.90% purity) as a yellow solid. ¹H NMR (400 MHz, DMSO-d₆) δ 10.94-10.85 (m, 1H), 8.51-8.45 (m, 2H), 7.87-7.81 (m, 2H), 7.70-7.63 (m, 2H), 7.55-7.50 (m, 1H), 7.47-7.40 (m, 1H), 6.77-6.71 (m, 1H), 3.93-3.88 (m, 2H), 3.78 (br s, 2H), 3.57-3.18 (m, 2H), 1.96-1.82 (m, 3H), 0.87 (br s, 2H), 0.53 (br s, 2H). HPLC: 98.90% (220 nm), 99.38% (215 nm), 99.74% (254 nm). MS (ESI): mass calcd. For C₂₅H₂₃F₃N₆O₃S 544.15, m/z found 545.2 [M+H]⁺.

### Compound 135: 4-chloro-1-[[6-(1, 1-difluoroethyl)-3-pyridyl]sulfonyl]-3-[7-(5-fluoropyrimidin-2-yl)-4, 7-diazaspiro [2.5]octan-4-yl]indazole

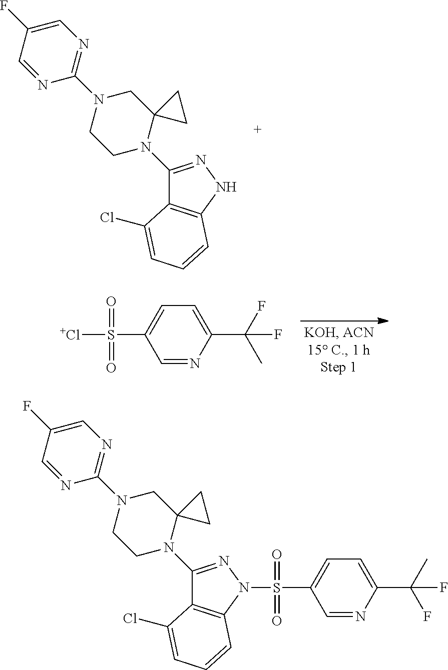 [SMILES: Fc1cnc(N2CCN(c3n[nH]c4cccc(Cl)c34)C3(CC3)C2)nc1 ; CC(F)(F)c1ccc(S(=O)(=O)[ClH+])cn1 ; CC(F)(F)c1ccc(S(=O)(=O)n2nc(N3CCN(c4ncc(F)cn4)CC34CC4)c3c(Cl)cccc32)cn1]

Step 1: To a solution of 4-chloro-3-[7-(5-fluoropyrimidin-2-yl)-4, 7-diazaspiro [2.5]octan-4-yl]-1H-indazole (20 mg, 55.7 μmol, 1 eq) and 6-(1,1-difluoroethyl)pyridine-3-sulfonyl chloride (26.9 mg, 112 μmol, 2 eq) in ACN (1 mL) was added KOH (9.38 mg, 167 μmol, 3 eq). The mixture was stirred at 15° C. for 1 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether/EtOAc=3/1) to give desired 4-chloro-1-[[6-(1, 1-difluoroethyl)-3-pyridyl]sulfonyl]-3-[7-(5-fluoropyrimidin-2-yl)-4, 7-diazaspiro [2.5]octan-4-yl]indazole (13 mg, 22.2 μmol, 39.8% yield, 96.29% purity) as a white solid. ¹H NMR (400 MHz, DMSO-d₆) δ 9.06 (s, 1H), 8.48-8.44 (m, 2H), 8.36 (dd, J=1.9, 8.3 Hz, 1H), 8.09 (d, J=8.4 Hz, 1H), 7.85 (d, J=8.4 Hz, 1H), 7.66 (t, J=8.1 Hz, 1H), 7.49 (d, J=7.8 Hz, 1H), 3.93 (s, 2H), 3.55 (br s, 4H), 1.91 (t, J=19.3 Hz, 3H), 0.85-0.75 (m, 2H), 0.39 (br s, 2H). HPLC: 96.29% (220 nm), 96.20% (215 nm), 97.55% (254 nm). MS (ESI): mass calcd. For C₂₄H₂₁ClF₃N₇O₂S 563.1, m/z found 564.1 [M+H]⁺.

### Compound 136: 4-chloro-1-[5-(1, 1-difluoroethyl) pyrimidin-2-yl]-3-[7-(5-fluoropyrimidin-2-yl)-4, 7-diazaspiro [2.5]octan-4-yl]indazole

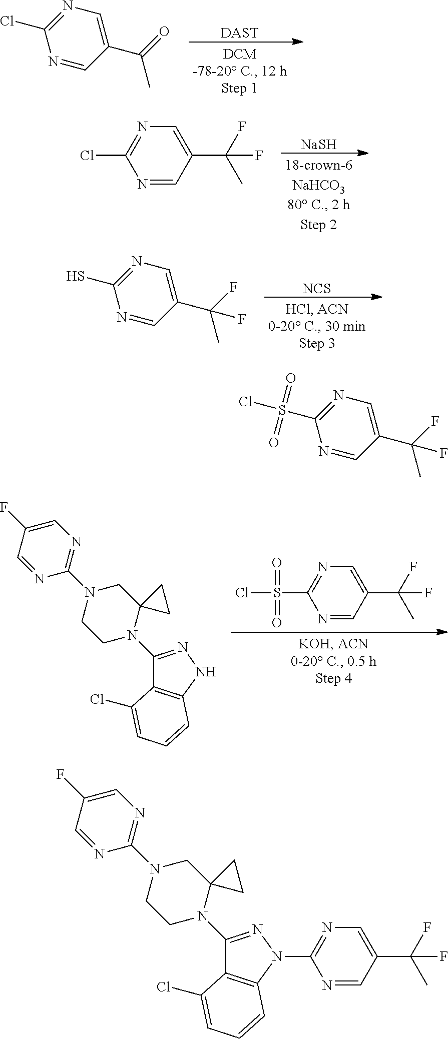

Step 1: To a solution of 1-(2-chloropyrimidin-5-yl)ethanone (2 g, 12.8 mmol, 1 eq) in DCM (50 mL) was added DAST (10.3 g, 63.9 mmol, 8.44 mL, 5 eq) at −78° C. The mixture was stirred at 20° C. for 12 hours. LC-MS showed 1-(2-chloropyrimidin-5-yl)ethanone was consumed completely and one main peak with desired mass was detected. The reaction mixture was added to sat. NaHCO₃ aq. (20 mL), extracted with DCM (10 mL*3). The combined organic layers were washed with brine (20 mL) and dried over Na₂SO₄, filtered and concentrated under reduced pressure to give a residue. The residue was purified by column chromatography (SiO₂, petroleum ether/Ethyl acetate=1/0 to 0/1) to give desired 2-chloro-5-(1, 1-difluoroethyl) pyrimidine (1.87 g, crude) as a white solid. MS (ESI): mass calcd. For C₆H₅ClF₂N₂ 178.01 m/z found 179.0 [M+H]⁺.

Step 2: To a solution of 2-chloro-5-(1,1-difluoroethyl)pyrimidine (800 mg, 4.48 mmol, 1 eq) and NaHS (628 mg, 11.2 mmol, 2.5 eq) in ACN (9 mL) was added 1, 4, 7, 10, 13-pentaoxacyclopentadecane (19.7 mg, 89.6 μmol, 17.7 μL, 0.02 eq) and benzyl(triethyl)ammonium;chloride (30.6 mg, 134 μmol, 0.03 eq). The mixture was stirred at 80° C. for 2 hours. LC-MS showed 5-bromo-2-(1, 1-difluoroethyl) pyrimidine was consumed completely and desired mass was detected. The reaction mixture was partitioned between water (10 mL) and EtOAc (20 mL). The organic phase was separated, washed with brine (20 mL), dried over Na₂SO₄, filtered and concentrated under reduced pressure to give a residue. The reaction mixture was filtered and the filter cake was concentrated under reduced pressure to give desired 5-(1, 1-difluoroethyl) pyrimidine-2-thiol (1.1 g, 6.24 mmol, 72.3% yield) as a yellow oil. MS (ESI): mass calcd. For C₆H₆F₂N₂S 176.02 m/z found 177.0 [M+H]⁺.

Step 3: To a solution of 5-(1, 1-difluoroethyl) pyrimidine-2-thiol (500 mg, 2.84 mmol, 1 eq) and H₂O (128 mg, 7.09 mmol, 128 μL, 2.5 eq) in ACN (10 mL) and was added NCS (228 mg, 1.70 mmol, 0.6 eq) and benzyl (triethyl) ammonium; chloride (1.94 g, 8.51 mmol, 3 eq) at 0° C. The mixture was stirred at 20° C. for 0.5 hour. LC-MS showed 5-(1, 1-difluoroethyl) pyrimidine-2-thiol was consumed completely and one main peak with desired mass was detected. The reaction mixture was filtered and the filtrate was concentrated under reduced pressure to give a residue. The residue was purified by prep-TLC (SiO₂, petroleum ether/ethyl acetate=3/1) to give desired 5-(1, 1-difluoroethyl) pyrimidine-2-sulfonyl chloride (260 mg, 1.07 mmol, 18.9% yield) as a yellow solid. MS (ESI): mass calcd. For C₆H₅ClF₂N₂O₂S 241.97 m/z found 292.1 [M+H+49]⁺.

Step 4: To a solution of 5-(1,1-difluoroethyl)pyrimidine-2-sulfonyl chloride (20 mg, 82.4 μmol, 2 eq) and 4-chloro-3-[7-(5-fluoropyrimidin-2-yl)-4,7-diazaspiro [2.5]octan-4-yl]-1H-indazole (14.8 mg, 41.2 μmol, 1 eq) in ACN (1 mL) was added KOH (6.94 mg, 124 μmol, 3 eq) at 0° C. The mixture was stirred at 20° C. for 0.5 hour. LC-MS showed 4-chloro-3-[7-(5-fluoropyrimidin-2-yl)-4, 7-diazaspiro [2.5]octan-4-yl]-1H-indazole was consumed completely and desired mass was detected. The reaction mixture was concentrated to give the crude product. The residue was purified by prep-HPLC (TFA condition; column: Phenomenex Luna C18 75*30 mm*3 μm; mobile phase: [H₂O(0.1% TFA)-ACN]; gradient:65%-95% B over 8.0 min) to give desired 4-chloro-1-[5-(1, 1-difluoroethyl)pyrimidin-2-yl]-3-[7-(5-fluoropyrimidin-2-yl)-4, 7-diazaspiro [2.5]octan-4-yl]indazole (5.2 mg, 9.92 μmol, 24.1% yield, 95.53% purity) as a yellow solid. ¹H NMR (400 MHz, DMSO-d₆) δ 9.16 (s, 2H), 8.73 (d, J=8.4 Hz, 1H), 8.51 (s, 2H), 7.62 (t, J=8.1 Hz, 1H), 7.45 (d, J=7.5 Hz, 1H), 4.07 (br s, 2H), 3.87 (br s, 2H), 3.66 (br t, J=4.6 Hz, 2H), 2.14 (t, J=19.1 Hz, 3H), 0.84 (s, 2H), 0.68 (s, 2H). HPLC: 95.53% (220 nm), 98.45% (215 nm), 98.44% (254 nm). MS (ESI): mass calcd. For C₂₃H₂₀ClF₃N₈ 500.15 m/z found 501.1 [M+H]⁺.

### Compound 137: 4-chloro-1-[2-(1, 1-difluoroethyl) pyrimidin-5-yl]sulfonyl-3-[7-(5-fluoropyrimidin-2-yl)-4, 7-diazaspiro [2.5]octan-4-yl]indazole

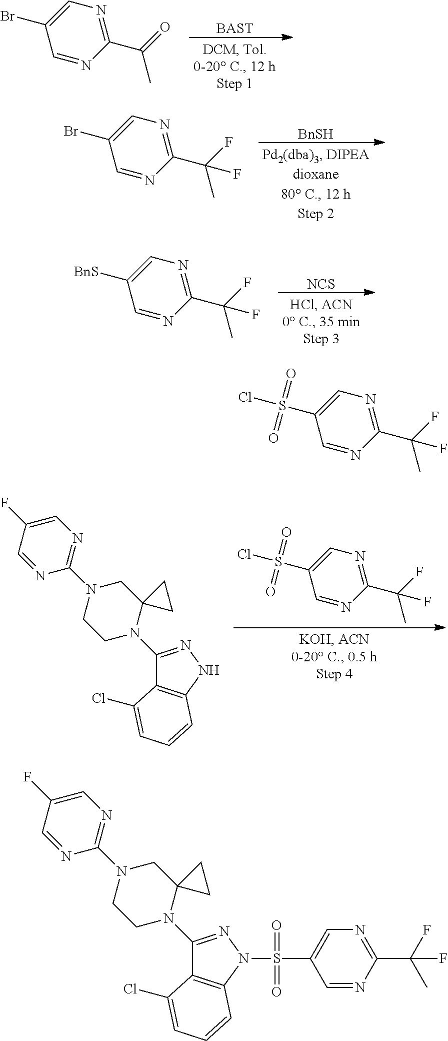

Step 1: To a solution of 1-(5-BROMOPYRIMIDIN-2-YL) ETHANONE (2 g, 9.95 mmol, 1 eq) in DCM (120 mL) and Tol. (10 mL) was added BAST (8.80 g, 39.8 mmol, 8.72 mL, 4 eq) at 0° C. under N₂. The mixture was stirred at 20° C. for 12 hours. LC-MS showed 1-(5-BROMOPYRIMIDIN-2-YL) ETHANONE was consumed completely and one main peak with desired mass was detected. Then it was separated between 30 mL of NaHCO₃ solution and 60 mL of EtOAc. The organic phase was separated, washed with 30 mL of brine, dried over Na₂SO₄, filtered and concentrated under reduced pressure to give the crude product. The residue was purified by flash silica gel chromatography (ISCO®; 2 g SepaFlash® Silica Flash Column, Eluent of 0˜20% ethyl acetate/petroleum ether gradient @60 mL/min) to give desired 5-bromo-2-(1, 1-difluoroethyl) pyrimidine (2.16 g, 9.69 mmol, 97.3% yield) as a yellow oil. MS (ESI): mass calcd. For C₆H₅BrF₂N₂ 221.96 m/z found 223.0 [M+H]⁺.

Step 2: A mixture of 5-bromo-2-(1, 1-difluoroethyl) pyrimidine (2 g, 8.97 mmol, 1 eq), phenylmethanethiol (1.67 g, 13.5 mmol, 1.58 mL, 1.5 eq), DIEA (2.32 g, 17.9 mmol, 3.12 mL, 2 eq), Xantphos (519 mg, 897 μmol, 0.1 eq) and Pd₂(dba)₃ (821 mg, 897 μmol, 0.1 eq) in dioxane (11 mL) was degassed and purged with N₂ for 3 times, and then the mixture was stirred at 80° C. for 12 hours under N₂ atmosphere. LC-MS showed 5-bromo-2-(1, 1-difluoroethyl) pyrimidine was consumed completely and desired mass was detected. The reaction mixture was partitioned between water (10 mL) and EtOAc (20 mL). The organic phase was separated, washed with brine (20 mL), dried over Na₂SO₄, filtered and concentrated under reduced pressure to give a residue. The residue was purified by prep-HPLC (TFA condition; column: Phenomenex luna C18 250*50 mm*10 um; mobile phase: [H₂O(0.1% TFA)-ACN];gradient:40%-70% B over 10.0 min) to give desired 5-benzylsulfanyl-2-(1,1-difluoroethyl)pyrimidine (2.5 g, crude) was obtained as a yellow oil. MS (ESI): mass calcd. For C₁₃H₁₂F₂N₂S 266.07 m/z found 267.1 [M+H]⁺.

Step 3: To a solution of HCl (0.3 mL) in ACN (3 mL) was added NCS (602 mg, 4.51 mmol, 4 eq) at 0° C. The mixture was stirred at 0° C. for 5 min. 5-benzylsulfanyl-2-(1, 1-difluoroethyl) pyrimidine (300 mg, 1.13 mmol, 1 eq) in ACN (3 mL) was added dropwise to the reaction. The mixture was stirred at 0° C. for 30 min. LC-MS showed 5-benzylsulfanyl-2-(1, 1-difluoroethyl) pyrimidine was consumed completely and one main peak with desired mass was detected. The reaction mixture was concentrated to give the crude product. The reaction mixture was added to water (20 mL), extracted with EtOAc (10 mL*3). The combined organic layers were washed with brine (20 mL) and dried over Na₂SO₄, filtered and concentrated under reduced pressure to give desired 2-(1,1-difluoroethyl)pyrimidine-5-sulfonyl chloride (270 mg, crude) as a white solid. MS (ESI): mass calcd. For C₆H₅ClF₂N₂O₂S 241.97 m/z found 292.1 [M+H+49]⁺.

Step 4: To a solution of 2-(1,1-difluoroethyl)pyrimidine-5-sulfonyl chloride (67.6 mg, 279 μmol, 5 eq) and 4-chloro-3-[7-(5-fluoropyrimidin-2-yl)-4,7-diazaspiro [2.5]octan-4-yl]-1H-indazole (20 mg, 55.7 μmol, 1 eq) in ACN (1 mL) was added KOH (9.38 mg, 167 μmol, 3 eq) at 0° C. The mixture was stirred at 20° C. for 0.5 hour. LC-MS showed 4-chloro-3-[7-(5-fluoropyrimidin-2-yl)-4, 7-diazaspiro [2.5]octan-4-yl]-1H-indazole remained and desired compound was detected. The reaction mixture was added HCl (1 M) until pH˜3, then the reaction mixture was added to water (5 mL), extracted with EtOAc (5 mL*3). The combined organic layers were washed with brine (5 mL) and dried over Na₂SO₄, filtered and concentrated under reduced pressure to give a residue. The residue was purified by prep-HPLC (TFA condition; column: Phenomenex Luna C18 75*30 mm*3 um; mobile phase: [H₂O(0.1% TFA)-ACN];gradient:60%-855% B over 8.0 min) to give desired 4-chloro-1-[2-(1,1-difluoroethyl)pyrimidin-5-yl]sulfonyl-3-[7-(5-fluoropyrimidin-2-yl)-4,7-diazaspiro[2.5]octan-4-yl]indazole (3 mg, 5.10 μmol, 9.15% yield, 96.07% purity) as a white solid. ¹H NMR (400 MHz, DMSO-d₆) δ 9.38 (s, 2H), 8.48 (s, 2H), 8.10 (br d, J=8.5 Hz, 1H), 7.67 (br t, J=8.0 Hz, 1H), 7.51 (br d, J=7.8 Hz, 1H), 3.96 (br s, 2H), 3.59 (br s, 4H), 2.01-1.91 (m, 3H), 0.80 (br s, 2H), 0.47 (br s, 2H). HPLC: 96.07% (220 nm), 96.61% (215 nm), 97.07% (254 nm). MS (ESI): mass calcd. For C₂₃H₂₀ClF₃N₈O₂S 564.11 m/z found 565.2 [M+H]⁺.

### Compound 138: 4-[4-chloro-1-(1-isopropylsulfonylpyrrol-3-yl) sulfonyl-indazol-3-yl]-3, 5-dimethyl-morpholine

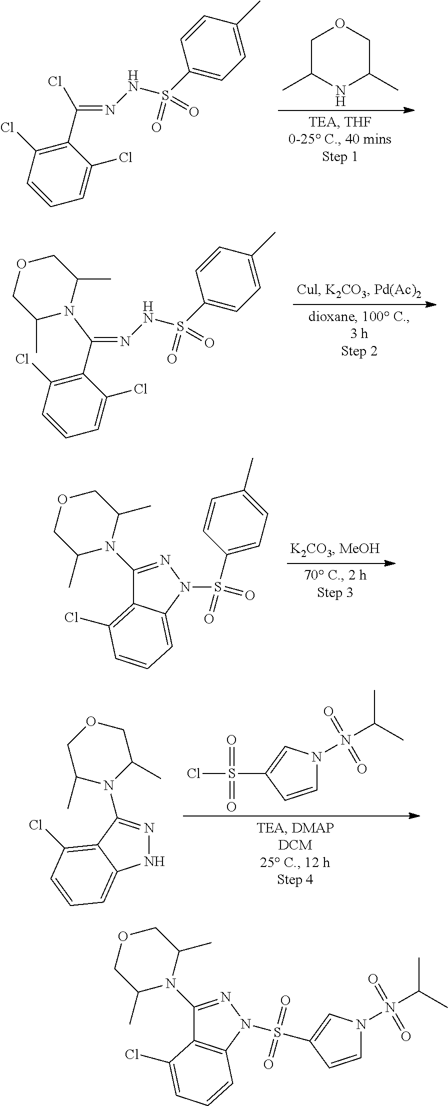

Step 1: To a solution of 3, 5-dimethylmorpholine (91.5 mg, 794 μmol, 1 eq) in THF (0.5 mL) was added dropwise TEA (121 mg, 1.19 mmol, 166 μL, 1.5 eq) at 25° C. After addition, the mixture was stirred at 25° C. for 10 mins, and then (1Z)-2, 6-dichloro-N-(p-tolylsulfonyl)benzohydrazonoyl chloride (300 mg, 794 μmol, 1 eq) in TEA (2 mL) was added dropwise at 0° C. The resulting mixture was stirred at 25° C. for 0.5 hour. LC-MS showed (1Z)-2, 6-dichloro-N-(p-tolylsulfonyl) benzohydrazonoyl chloride was consumed completely and one main peak with desired mass was detected. Then it was separated between water (20 mL) and EtOAc (40 mL). The organic phase was separated, washed with brine (30 mL), dried over Na₂SO₄, filtered and concentrated under reduced pressure to give desired N—[(Z)-[(2, 6-dichlorophenyl)-(3, 5-dimethylmorpholin-4-yl) methylene]amino]-4-methyl-benzenesulfonamide (400 mg, crude) as a yellow solid. MS (ESI): mass calcd. For C₂₀H₂₃Cl₂N₃O₃S 455.08, m/z found 456.2 [M+H]⁺.

Step 2: A mixture of N—[(Z)-[(2, 6-dichlorophenyl)-(3, 5-dimethylmorpholin-4-yl) methylene]amino]-4-methyl-benzenesulfonamide (400 mg, 876 μmol, 1 eq), CuI (16.7 mg, 87.7 μmol, 0.1 eq), Pd(OAc)₂ (39.4 mg, 175 μmol, 0.2 eq) and K₂CO₃ (303 mg, 2.19 mmol, 2.5 eq) in dioxane (5 mL) was degassed and purged with N₂ for 3 times, and then the mixture was stirred at 100° C. for 3 hours under N₂ atmosphere. LC-MS showed N—[(Z)-[(2, 6-dichlorophenyl)-(3, 5-dimethylmorpholin-4-yl) methylene]amino]-4-methyl-benzenesulfonamide remained and desired compound was detected. Then it was separated between water (5 mL) and EtOAc (10 mL). The organic phase was separated, washed with brine (10 mL), dried over Na₂SO₄, filtered and concentrated under reduced pressure to give desired 4-[4-chloro-1-(p-tolylsulfonyl) indazol-3-yl]-3, 5-dimethyl-morpholine (400 mg, crude) as a yellow oil. MS (ESI): mass calcd. For C₂₀H₂₂ClN₃O₃S 419.11, m/z found 420.1 [M+H]⁺.

Step 3: To a solution of 4-[4-chloro-1-(p-tolylsulfonyl) indazol-3-yl]-3, 5-dimethyl-morpholine (2.3 g, 5.48 mmol, 1 eq) in MeOH (20 mL) was added K₂CO₃ (3.79 g, 27.4 mmol, 5 eq). The mixture was stirred at 70° C. for 2 hours. LC-MS showed 4-[4-chloro-1-(p-tolylsulfonyl) indazol-3-yl]-3, 5-dimethyl-morpholine remained and the desired compound was detected. The reaction mixture was added to water (20 mL), extracted with EtOAc (30 mL). The combined organic layers were washed with brine (20 mL) and dried over Na₂SO₄, filtered and concentrated under reduced pressure to give a residue. The residue was purified by prep-TLC (SiO₂, petroleum ether/EtOAc=3/1) to give desired 4-(4-chloro-1H-indazol-3-yl)-3, 5-dimethyl-morpholine (450 mg, crude) as a yellow oil. MS (ESI): mass calcd. For C₁₃H₁₆ClN₃O 265.10, m/z found 266.1 [M+H]⁺.

Step 4: To a solution of 4-(4-chloro-1H-indazol-3-yl)-3, 5-dimethyl-morpholine (100 mg, 376 μmol, 1 eq) and 1-isopropylsulfonylpyrrole-3-sulfonyl chloride (205 mg, 753 μmol, 2 eq) in DCM (1 mL) was added TEA (76.2 mg, 753 μmol, 105 μL, 2 eq) and DMAP (4.60 mg, 37.6 μmol, 0.1 eq). The mixture was stirred at 25° C. for 12 hours. LC-MS showed 4-(4-chloro-1H-indazol-3-yl)-3, 5-dimethyl-morpholine was consumed completely and desired mass was detected. The reaction mixture was added to water (20 mL), extracted with DCM (30 mL). The combined organic layers were washed with brine (20 mL) and dried over Na₂SO₄, filtered and concentrated under reduced pressure to give a residue. The residue was purified by prep-HPLC (neutral condition; column: Waters Xbridge Prep OBD C18 150*40 mm*10 μm; mobile phase: [water(NH₄HCO₃)-ACN]; B %: 55%-85%, 8 mins) to give desired 4-[4-chloro-1-(1-isopropylsulfonylpyrrol-3-yl) sulfonyl-indazol-3-yl]-3, 5-dimethyl-morpholine (30.4 mg, 57 μmol, 15.1% yield, 93.82% purity) as a white solid. ¹H NMR (400 MHz, DMSO-d₆) δ 8.05 (d, J=8.6 Hz, 1H), 7.93 (t, J=2.0 Hz, 1H), 7.59 (t, J=8.1 Hz, 1H), 7.43 (d, J=7.7 Hz, 1H), 7.31 (dd, J=2.5, 3.2 Hz, 1H), 6.44 (dd, J=1.6, 3.4 Hz, 1H), 3.94-3.85 (m, 1H), 3.84-3.73 (m, 2H), 3.72-3.55 (m, 3H), 3.19-3.05 (m, 1H), 1.05 (dd, J=2.2, 6.8 Hz, 6H), 0.89-0.81 (m, 3H), 0.77 (br dd, J=1.1, 2.2 Hz, 3H). HPLC: 90.22% (220 nm), 89.17% (215 nm), 93.82% (254 nm). MS (ESI): mass calcd. For C₂₀H₂₅ClN₄O₅S₂500.10, m/z found 501.1 [M+H]⁺.

### Compound 139: 3-(5-azaspiro [2.5]octan-5-yl)-4-chloro-1-(1-isopropylsulfonylpyrrol-3-yl) sulfonyl-indazole

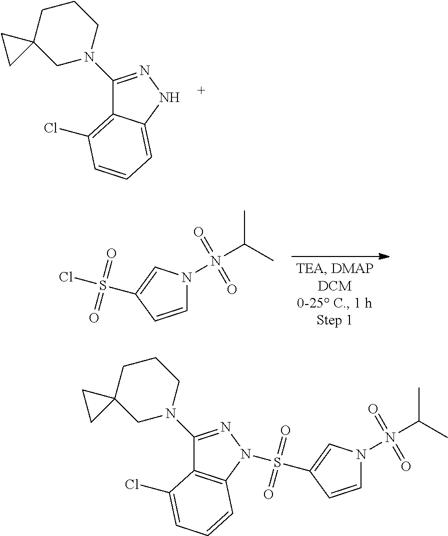

Step 1: To a solution of 3-(5-azaspiro [2.5]octan-5-yl)-4-chloro-1H-indazole (100 mg, 382 μmol, 1 eq) and 1-isopropylsulfonylpyrrole-3-sulfonyl chloride (208 mg, 764 μmol, 2 eq) in DCM (1 mL) was added TEA (77.3 mg, 764 μmol, 106 μL, 2 eq) and DMAP (4.67 mg, 38.2 μmol, 0.1 eq) at 0° C. The mixture was stirred at 25° C. for 1 hour. LC-MS showed 3-(5-azaspiro [2.5]octan-5-yl)-4-chloro-1H-indazole was consumed completely and desired mass was detected. The reaction mixture was added to water (20 mL), extracted with EtOAc (30 mL). The combined organic layers were washed with brine (20 mL) and dried over Na₂SO₄, filtered and concentrated under reduced pressure to give a residue. The residue was purified by prep-HPLC (neutral condition; column: Waters Xbridge Prep OBD C18 150*40 mm*10 μm; mobile phase: [water(NH₄HCO₃)-ACN]; B %: 60%-90%, 8 mins) to give desired 3-(5-azaspiro [2.5]octan-5-yl)-4-chloro-1-(1-isopropylsulfonylpyrrol-3-yl) sulfonyl-indazole (56.7 mg, 113 μmol, 29.5% yield, 98.84% purity) as a pale yellow solid. ¹H NMR (400 MHz, DMSO-d₆) δ 7.98 (d, J=8.3 Hz, 1H), 7.83 (t, J=2.0 Hz, 1H), 7.56 (t, J=8.1 Hz, 1H), 7.41 (d, J=7.7 Hz, 1H), 7.28 (dd, J=2.4, 3.3 Hz, 1H), 6.46 (dd, J=1.6, 3.4 Hz, 1H), 3.89 (t, J=6.8 Hz, 1H), 3.24-3.13 (m, 2H), 2.96 (s, 2H), 1.76 (br dd, J=1.0, 4.9 Hz, 2H), 1.41 (br dd, J=4.5, 6.0 Hz, 2H), 1.02 (d, J=6.6 Hz, 6H), 0.35-0.30 (m, 2H), 0.27 (s, 2H). HPLC: 98.84% (220 nm), 98.49% (215 nm), 96.65% (254 nm). MS (ESI): mass calcd. For C₂₀H₂₅ClN₄O₅S₂ 496.10, m/z found 497.1 [M+H]⁺.

### Compound 140: 4-chloro-3-[7-(5-chloro-3-fluoro-2-pyridyl)-4, 7-diazaspiro [2.5]octan-4-yl]-1-(1-isopropylsulfonylpyrrol-3-yl) sulfonyl-indazole

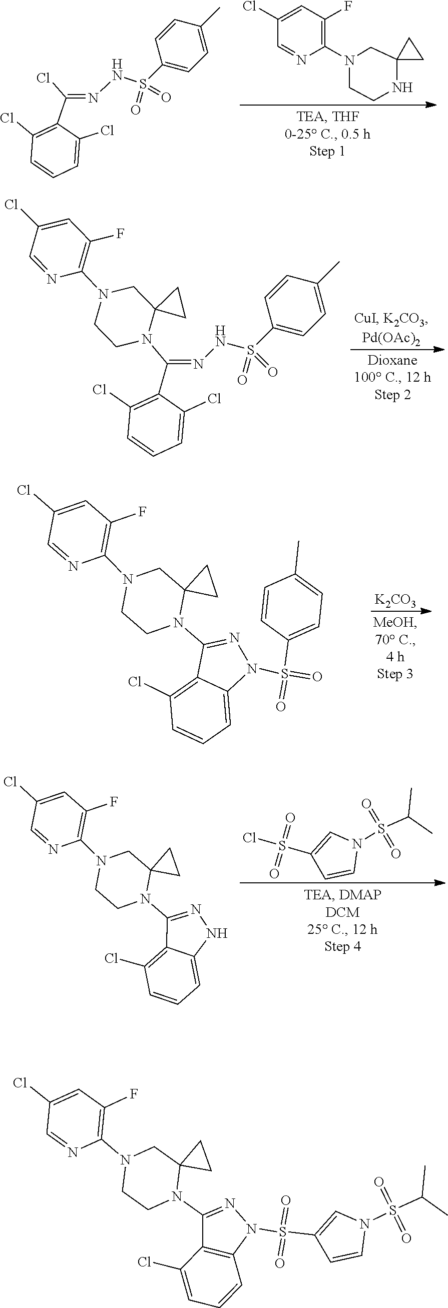

Step 1: To a solution of 7-(5-chloro-3-fluoro-2-pyridyl)-4,7-diazaspiro[2.5]octane (250 mg, 899 μmol, 1 eq, HCl) in THF (3 mL) was added dropwise TEA (182 mg, 1.80 mmol, 250 μL, 2 eq) at 25° C. After addition, the mixture was stirred 25° C. for 15 mins, and then (1Z)-2,6-dichloro-N-(p-tolylsulfonyl)benzohydrazonoyl chloride (373 mg, 989 μmol, 1.1 eq) in THF (3 mL) was added dropwise at 0° C. The resulting mixture was stirred at 25° C. for 15 mins. LC-MS showed 7-(5-chloro-3-fluoro-2-pyridyl)-4, 7-diazaspiro [2.5]octane was consumed completely and one main peak with desired m/z was detected. The reaction mixture was partitioned between EtOAc (8 mL) and H₂O (5 mL). The organic phase was separated, concentrated under reduced pressure to give desired N—[(Z)-[[7-(5-chloro-3-fluoro-2-pyridyl)-4, 7-diazaspiro [2.5]octan-4-yl]-(2, 6-dichlorophenyl) methylene]amino]-4-methyl-benzenesulfonamide (500 mg, crude) as a yellow solid.

Step 2: A mixture of N—[(Z)—[[7-(5-chloro-3-fluoro-2-pyridyl)-4, 7-diazaspiro [2.5]octan-4-yl]-(2, 6-dichlorophenyl) methylene]amino]-4-methyl-benzenesulfonamide (500 mg, 858 μmol, 1 eq), CuI (16.3 mg, 85.8 μmol, 0.1 eq), K₂CO₃ (296 mg, 2.14 mmol, 2.5 eq) and Pd(OAc)₂ (38.5 mg, 172 μmol, 0.2 eq) in dioxane (6 mL) was degassed and purged with N₂ for 3 times, and then the mixture was stirred at 100° C. for 12 hours under N₂ atmosphere. TLC (petroleum ether/EtOAc=3/1) indicated N—[(Z)—[[7-(5-chloro-3-fluoro-2-pyridyl)-4, 7-diazaspiro [2.5]octan-4-yl]-(2, 6-dichlorophenyl) methylene]amino]-4-methyl-benzenesulfonamide was consumed completely and one new spot formed. The reaction mixture was partitioned between EtOAc (20 mL) and H₂O (10 mL). The organic phase was separated, dried over anhydrous Na₂SO₄, filtered and concentrated under reduced pressure to give desired 4-chloro-3-[7-(5-chloro-3-fluoro-2-pyridyl)-4, 7-diazaspiro [2.5]octan-4-yl]-1-(p-tolylsulfonyl) indazole (450 mg, crude) as a white solid.

Step 3: To a solution of 4-chloro-3-[7-(5-chloro-3-fluoro-2-pyridyl)-4, 7-diazaspiro [2.5]octan-4-yl]-1-(p-tolylsulfonyl) indazole (225 mg, 412 μmol, 1 eq) in MeOH (4 mL) was added K₂CO₃ (285 mg, 2.06 mmol, 5 eq). The mixture was stirred at 70° C. for 4 hours. LC-MS showed 4-chloro-3-[7-(5-chloro-3-fluoro-2-pyridyl)-4, 7-diazaspiro [2.5]octan-4-yl]-1-(p-tolylsulfonyl) indazole was consumed completely and one main peak with desired m/z was detected. The reaction mixture was concentrated under reduced pressure to remove MeOH. The residue was diluted with H₂O (10 mL) and extracted with DCM (30 mL). The combined organic layers were dried over anhydrous Na₂SO₄, filtered and concentrated under reduced pressure to give a residue. The residue was purified by prep-TLC (SiO₂, petroleum ether/EtOAc=3/1) to give desired 4-chloro-3-[7-(5-chloro-3-fluoro-2-pyridyl)-4, 7-diazaspiro [2.5]octan-4-yl]-1H-indazole (200 mg, 510 μmol, 61.9% yield) as a yellow oil. MS (ESI): mass calcd. For C₁₈H₁₆Cl₂FN₅ 391.08, m/z found 392.1 [M+H]⁺.

Step 4: To a solution of 4-chloro-3-[7-(5-chloro-3-fluoro-2-pyridyl)-4,7-diazaspiro[2.5]octan-4-yl]-1H-indazole (50 mg, 127 μmol, 1 eq) and I-isopropylsulfonylpyrrole-3-sulfonyl chloride (52.0 mg, 191 μmol, 1.5 eq) in DCM (2 mL) was added TEA (25.8 mg, 255 μmol, 35.5 μL, 2 eq) and DMAP (1.56 mg, 12.8 μmol, 0.1 eq). The mixture was stirred at 25° C. for 12 hours. LC-MS showed 4-chloro-3-[7-(5-chloro-3-fluoro-2-pyridyl)-4, 7-diazaspiro [2.5]octan-4-yl]-1H-indazole was consumed completely and one main peak with desired m/z was detected. The reaction mixture was partitioned between DCM (3 mL) and H₂O (2 mL). The organic phase was separated, dried over anhydrous Na₂SO₄, filtered and concentrated under reduced pressure to give a residue. The residue was purified by prep-TLC (SiO₂, petroleum ether/EtOAc=2/1) to give desired 4-chloro-3-[7-(5-chloro-3-fluoro-2-pyridyl)-4, 7-diazaspiro [2.5]octan-4-yl]-1-(1-isopropylsulfonylpyrrol-3-yl) sulfonyl-indazole (25 mg, 37.6 μmol, 29.5% yield, 94.40% purity) as a yellow solid. ¹H NMR (400 MHz, DMSO-d₆) δ 0.49 (br s, 2H) 0.79 (br s, 2H) 1.04 (d, J=6.75 Hz, 6H) 3.45-3.46 (m, 2H) 3.63 (brt, J=4.75 Hz, 4H) 3.87-3.96 (m, 1H) 6.49 (dd, J=3.31, 1.56 Hz, 1H) 7.33 (t, J=2.81 Hz, 1H) 7.45 (d, J=7.63 Hz, 1H) 7.60 (t, J=8.13 Hz, 1H) 7.83 (dd, J=12.88, 2.13 Hz, 1H) 7.87 (t, J=1.88 Hz, 1H) 8.03 (d, J=8.38 Hz, 1H) 8.09 (d, J=1.88 Hz, 1H). HPLC: 94.40% (220 nm), 94.66% (215 nm), 94.79% (254 nm). MS (ESI): mass calcd. For C₂₅H₂₅Cl₂FN₆O₄S₂ 626.07 m/z found 627.1 [M+H]⁺.

### Compound 141: 4-chloro-3-[7-(3, 5-difluoro-2-pyridyl)-4, 7-diazaspiro [2.5]octan-4-yl]-1-(1-isopropylsulfonylpyrrol-3-yl) sulfonyl-indazole

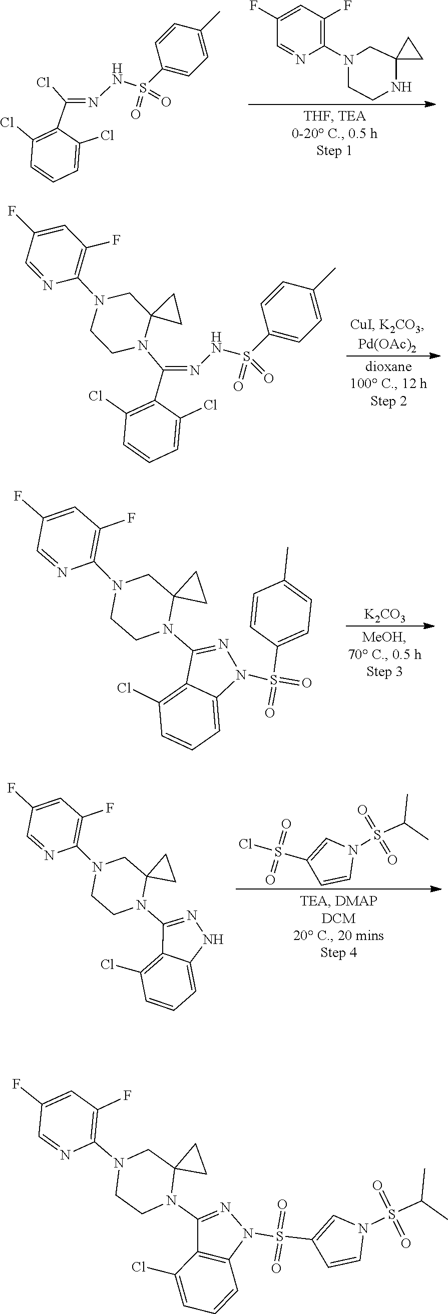

Step 1: To a solution of (1Z)-2, 6-dichloro-N-(p-tolylsulfonyl)benzohydrazonoyl chloride (649 mg, 1.72 mmol, 1.5 eq) in THF (3 mL) was added dropwise TEA (348 mg, 3.44 mmol, 3 eq) at 20° C. After addition, the mixture was stirred at this temperature for 5 mins, and then 7-(3, 5-difluoro-2-pyridyl)-4, 7-diazaspiro [2.5]octane (300 mg, 1.15 mmol, 1 eq, HCl) in THF (2 mL) was added dropwise at 0° C. The resulting mixture was stirred at 20° C. for 25 mins. LC-MS showed 7-(3, 5-difluoro-2-pyridyl)-4, 7-diazaspiro [2.5]octane was consumed completely and one main peak with desired mass was detected. The reaction mixture was added to water (90 mL), extracted with EtOAc (90 mL). The combined organic layers were washed with brine (90 mL) and dried over Na₂SO₄, filtered and concentrated under reduced pressure to give a residue. The residue was purified by prep-TLC (SiO₂, petroleum ether/EtOAc=3/1) to give desired N—[(Z)-[(2, 6-dichlorophenyl)-[7-(3, 5-difluoro-2-pyridyl)-4, 7-diazaspiro [2.5]octan-4-yl]methylene]amino]-4-methyl-benzenesulfonamide (260 mg, 459 μmol, 40.0% yield) as a yellow oil. MS (ESI): mass calcd. For C₂₅H₂₃N₅SO₂Cl₂F₂ 565.09, m/z found 566.0 [M+H]⁺.

Step 2: A mixture of N—[(Z)-[(2, 6-dichlorophenyl)-[7-(3, 5-difluoro-2-pyridyl)-4, 7-diazaspiro [2.5]octan-4-yl]methylene]amino]-4-methyl-benzenesulfonamide (260 mg, 459 μmol, 1 eq), CuI (8.74 mg, 45.9 μmol, 0.1 eq), Pd(OAc)₂ (20.6 mg, 91.8 μmol, 0.2 eq) and K₂CO₃ (159 mg, 1.15 mmol, 2.5 eq) in dioxane (3 mL) was degassed and purged with N₂ for 3 times, and then the mixture was stirred at 100° C. for 12 hours under N₂ atmosphere. LC-MS showed N—[(Z)-[(2, 6-dichlorophenyl)-[7-(3, 5-difluoro-2-pyridyl)-4, 7-diazaspiro [2.5]octan-4-yl]methylene]amino]-4-methyl-benzenesulfonamide was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give desired 4-chloro-3-[7-(3, 5-difluoro-2-pyridyl)-4, 7-diazaspiro [2.5]octan-4-yl]-1-(p-tolylsulfonyl) indazole (150 mg, crude) as a yellow solid. MS (ESI): mass calcd. For C₂₅H₂₂N₅SO₂ClF₂ 529.12, m/z found 530.2 [M+H]⁺.

Step 3: To a solution of 4-chloro-3-[7-(3, 5-difluoro-2-pyridyl)-4, 7-diazaspiro [2.5]octan-4-yl]-1-(p-tolylsulfonyl) indazole (150 mg, 283 μmol, 1 eq) in MeOH (4 mL) was added K₂CO₃ (196 mg, 1.42 mmol, 5 eq) was stirred at 70° C. for 0.5 hour. LC-MS showed a little of 4-chloro-3-[7-(3, 5-difluoro-2-pyridyl)-4, 7-diazaspiro [2.5]octan-4-yl]-1-(p-tolylsulfonyl) indazole remained. Several new peaks were shown on LC-MS and desired compound was detected. Then it was separated between water (5 mL) and EtOAc (10 mL). The organic phase was separated, washed with brine (5 mL), dried over Na₂SO₄, filtered and concentrated under reduced pressure to give the crude product. The residue was purified by prep-TLC (SiO₂, petroleum ether/EtOAc=3/1) to give desired 4-chloro-3-[7-(3, 5-difluoro-2-pyridyl)-4, 7-diazaspiro [2.5]octan-4-yl]-1H-indazole (25 mg, 66.5 μmol, 23.5% yield) as a yellow solid. MS (ESI): mass calcd. For C₁₈H₁₆N₅ClF₂ 375.11, m/z found 376.0 [M+H]⁺.

Step 4: A mixture of 4-chloro-3-[7-(3, 5-difluoro-2-pyridyl)-4, 7-diazaspiro [2.5]octan-4-yl]-1H-indazole (20 mg, 53.2 μmol, 1 eq), 1-isopropylsulfonylpyrrole-3-sulfonyl chloride (21.7 mg, 79.8 μmol, 1.5 eq), TEA (5.39 mg, 53.2 μmol, 1 eq) and DMAP (6.50 mg, 53.2 μmol, 1 eq) in DCM (1 mL) was degassed and purged with N₂ for 3 times, and then the mixture was stirred at 20° C. for 20 mins under N₂ atmosphere. LC-MS showed 4-chloro-3-[7-(3, 5-difluoro-2-pyridyl)-4, 7-diazaspiro [2.5]octan-4-yl]-1H-indazole was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-HPLC (neutral condition; Method: column: Waters Xbridge Prep OBD C18 150*40 mm*10 um; mobile phase: [water (NH₄HCO₃)-ACN]; B %: 60%-90%, 8 min) to give desired 4-chloro-3-[7-(3, 5-difluoro-2-pyridyl)-4, 7-diazaspiro [2.5]octan-4-yl]-1-(1-isopropylsulfonylpyrrol-3-yl) sulfonyl-indazole (7 mg, 11.4 μmol, 21.4% yield, 99.43% purity) as a yellow solid. ¹H NMR (400 MHz, DMSO-d₆) δ 8.13-8.08 (m, 1H), 8.03 (d, J=8.5 Hz, 1H), 7.88-7.84 (m, 1H), 7.84-7.76 (m, 1H), 7.63-7.57 (m, 1H), 7.47-7.42 (m, 1H), 7.34-7.30 (m, 1H), 6.50-6.46 (m, 1H), 3.95-3.86 (m, 1H), 3.67-3.60 (m, 2H), 3.40-3.34 (m, 4H), 1.07-1.01 (m, 6H), 0.81-0.76 (m, 2H), 0.52-0.45 (m, 2H). HPLC: 99.43% (220 nm), 99.54% (215 nm), 99.07% (254 nm). MS (ESI): mass calcd. For C₂₅H₂₅N₆S₂O₄ClF₂ 610.10 m/z found 611.3 [M+H]⁺.

### Compound 142: 4-chloro-3-[7-(3, 5-difluoro-2-pyridyl)-4, 7-diazaspiro [2.5]octan-4-yl]-7-fluoro-1-(1-isopropylsulfonylpyrrol-3-yl) sulfonyl-indazole

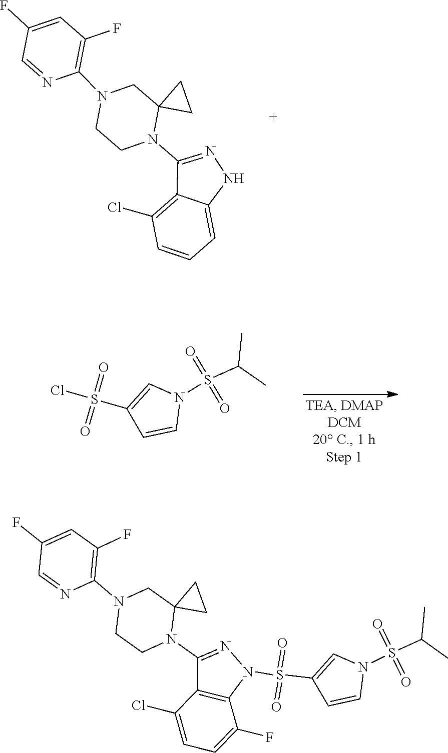 [SMILES: Fc1cnc(N2CCN(c3n[nH]c4cccc(Cl)c34)C3(CC3)C2)c(F)c1 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cc4F)CC34CC4)c3c(Cl)ccc(F)c32)c1]

Step 1: To a solution of 4-chloro-3-[7-(3, 5-difluoro-2-pyridyl)-4, 7-diazaspiro [2.5]octan-4-yl]-7-fluoro-1H-indazole (100 mg, 254 μmol, 1 eq) and 1-isopropylsulfonylpyrrole-3-sulfonyl chloride (82.8 mg, 305 μmol, 1.2 eq) in DCM (2 mL) was added TEA (51.4 mg, 508 μmol, 70.7 μL, 2 eq) and DMAP (3.10 mg, 25.4 μmol, 0.1 eq). The mixture was stirred at 20° C. for 1 hour. LC-MS showed 4-chloro-3-[7-(3, 5-difluoro-2-pyridyl)-4, 7-diazaspiro [2.5]octan-4-yl]-7-fluoro-1H-indazole was consumed completely and desired mass was detected. The crude was added H₂O (10 mL), and extracted with EtOAc (15 mL*3). The combined organic layers were washed with brine (10 mL), dried over Na₂SO₄, filtered and concentrated under reduced pressure to give a residue. The residue was purified by prep-TLC (SiO₂, petroleum ether/EtOAc=3/1) to give desired 4-chloro-3-[7-(3, 5-difluoro-2-pyridyl)-4, 7-diazaspiro [2.5]octan-4-yl]-7-fluoro-1-(1-isopropylsulfonylpyrrol-3-yl)sulfonyl-indazole(19.7 mg, 30.6 μmol, 12.1% yield, 97.78% purity) as a white solid. ¹H NMR (400 MHz, DMSO-d₆) δ 8.10 (d, J=2.5 Hz, 1H), 8.03 (dd, J=3.9, 9.1 Hz, 1H), 7.90 (t, J=2.0 Hz, 1H), 7.80 (ddd, J=2.5, 8.3, 12.5 Hz, 1H), 7.69 (t, J=9.3 Hz, 1H), 7.33 (dd, J=2.4, 3.3 Hz, 1H), 6.50 (dd, J=1.7, 3.3 Hz, 1H), 3.96-3.88 (m, 1H), 3.62 (br t, J=4.7 Hz, 2H), 3.59-3.50 (m, 2H), 3.36-3.28 (m, 2H), 1.05 (d, J=6.8 Hz, 6H), 0.78 (br s, 2H), 0.49 (br s, 2H). HPLC: 97.78% (220 nm), 97.85% (215 nm), 98.28% (254 nm). MS (ESI): mass calcd. For C₂₅H₂₄ClF₃N₆O₄S₂ 628.09 m/z found 629.0 [M+H]⁺.

### Compound 143: 4-chloro-1-((1-(cyclopropylsulfonyl)-1H-pyrrol-3-yl) sulfonyl)-3-(7-(3, 5-difluoropyridin-2-yl)-4, 7-diazaspiro [2.5]octan-4-yl)-7-fluoro-1H-indazole

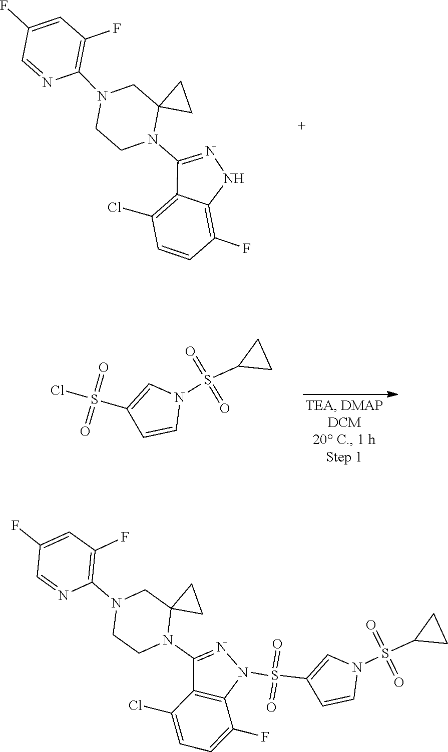 [SMILES: Fc1cnc(N2CCN(c3n[nH]c4c(F)ccc(Cl)c34)C3(CC3)C2)c(F)c1 ; O=S(=O)(Cl)c1ccn(S(=O)(=O)C2CC2)c1 ; O=S(=O)(c1ccn(S(=O)(=O)C2CC2)c1)n1nc(N2CCN(c3ncc(F)cc3F)CC23CC3)c2c(Cl)ccc(F)c21]

Step 1: To a solution of 4-chloro-3-[7-(3, 5-difluoro-2-pyridyl)-4, 7-diazaspiro [2.5]octan-4-yl]-7-fluoro-1H-indazole (100 mg, 254 μmol, 1 eq) and 1-cyclopropylsulfonylpyrrole-3-sulfonyl chloride (82.2 mg, 305 μmol, 1.2 eq) in DCM (2 mL) was added TEA (51.4 mg, 508 μmol, 70.7 μL, 2 eq) and DMAP (3.10 mg, 25.4 μmol, 0.1 eq). The mixture was stirred at 20° C. for 1 hour. LC-MS showed 4-chloro-3-[7-(3, 5-difluoro-2-pyridyl)-4, 7-diazaspiro [2.5]octan-4-yl]-7-fluoro-1H-indazole was consumed completely and desired mass was detected. The crude was added H₂O (10 mL), and extracted with EtOAc (15 mL*3). The combined organic layers were washed with brine (10 mL), dried over Na₂SO₄, filtered and concentrated under reduced pressure to give a residue. The residue was purified by prep-HPLC (FA condition, column: Phenomenex Luna C18 75*30 mm*3 μm; mobile phase: [water (FA)-ACN]; B %: 65%-95%, 8 min) to give desired 4-chloro-1-((1-(cyclopropylsulfonyl)-1H-pyrrol-3-yl) sulfonyl)-3-(7-(3, 5-difluoropyridin-2-yl)-4, 7-diazaspiro [2.5]octan-4-yl)-7-fluoro-1H-indazole (30.6 mg, 48.5 μmol, 19.1% yield, 99.35% purity) as a white solid. ¹H NMR (400 MHz, DMSO-d₆) δ 8.13 (d, J=2.4 Hz, 1H), 8.05 (dd, J=3.9, 9.1 Hz, 1H), 8.01 (t, J=2.0 Hz, 1H), 7.88-7.80 (m, 1H), 7.73 (t, J=9.2 Hz, 1H), 7.39 (dd, J=2.4, 3.3 Hz, 1H), 6.47 (dd, J=1.7, 3.4 Hz, 1H), 3.68-3.63 (m, 2H), 3.63-3.48 (m, 2H), 3.35-3.30 (m, 2H), 3.29-3.25 (m, 1H), 1.32 (br dd, J=2.6, 4.2 Hz, 2H), 1.16 (dd, J=2.3, 7.6 Hz, 2H), 0.80 (br s, 2H), 0.50 (br s, 2H). HPLC: 99.35% (220 nm), 99.43% (215 nm), 99.15% (254 nm). MS (ESI): mass calcd. For C₂₅H₂₂ClF₃N₆O₄S₂ 626.08 m/z found 627.2 [M+H]⁺.

### Compound 144: 3-[7-(3, 5-difluoro-2-pyridyl)-4, 7-diazaspiro [2.5]octan-4-yl]-4, 7-difluoro-1-(1-isopropylsulfonylpyrrol-3-yl) sulfonyl-indazole

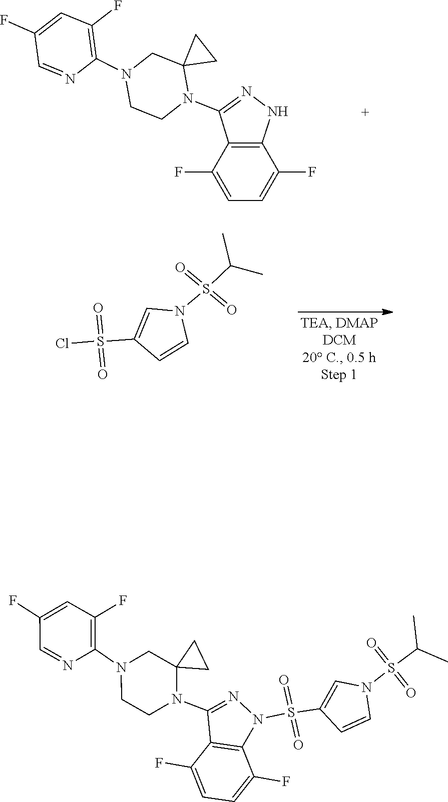

Step 1: To a mixture of 3-[7-(3, 5-difluoro-2-pyridyl)-4, 7-diazaspiro [2.5]octan-4-yl]-4, 7-difluoro-1H-indazole (30 mg, 79.5 μmol, 1 eq) and 1-isopropylsulfonylpyrrole-3-sulfonyl chloride (32.4 mg, 119 μmol, 1.5 eq) in DCM (1 mL) was added TEA (24.1 mg, 239 μmol, 3 eq) and DMAP (971 pg, 7.95 μmol, 0.1 eq), and then the mixture was stirred at 20° C. for 0.5 hour. LC-MS showed 3-[7-(3, 5-difluoro-2-pyridyl)-4, 7-diazaspiro [2.5]octan-4-yl]-4, 7-difluoro-1H-indazole was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-TLC (SiO₂, petroleum ether/EtOAc=3/1) to give desired 3-[7-(3, 5-difluoro-2-pyridyl)-4, 7-diazaspiro [2.5]octan-4-yl]-4, 7-difluoro-1-(1-isopropylsulfonylpyrrol-3-yl) sulfonyl-indazole (9.5 mg, 14.6 μmol, 18.4% yield, 94.48% purity) as a white solid. ¹H NMR (400 MHz, DMSO-d₆) δ 8.11-8.08 (m, 1H), 7.80 (br dd, J=2.6, 4.2 Hz, 1H), 7.78-7.75 (m, 1H), 7.58-7.50 (m, 1H), 7.34 (dd, J=2.4, 3.3 Hz, 1H), 7.27-7.18 (m, 1H), 6.50-6.45 (m, 1H), 4.02-3.88 (m, 1H), 3.73-3.65 (m, 2H), 3.57-3.42 (m, 2H), 3.29 (br s, 2H), 1.12-1.03 (m, 6H), 0.88-0.79 (m, 2H), 0.66-0.56 (m, 2H). HPLC: 94.48% (220 nm), 95.23% (215 nm), 95.71% (254 nm). MS (ESI): mass calcd. For C₂₅H₂₄N₆S₂O₄F₄ 612.12 m/z found 613.3 [M+H]⁺.

### Compound 145: 1-(1-cyclopropylsulfonylpyrrol-3-yl) sulfonyl-3-[7-(3, 5-difluoro-2-pyridyl)-4, 7-diazaspiro [2.5]octan-4-yl]-4, 7-difluoro-indazole

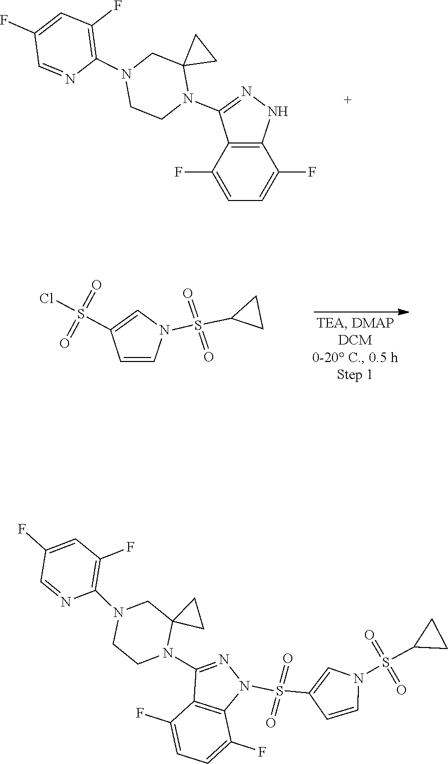 [SMILES: Fc1cnc(N2CCN(c3n[nH]c4c(F)ccc(F)c34)C3(CC3)C2)c(F)c1 ; O=S(=O)(Cl)c1ccn(S(=O)(=O)C2CC2)c1 ; O=S(=O)(c1ccn(S(=O)(=O)C2CC2)c1)n1nc(N2CCN(c3ncc(F)cc3F)CC23CC3)c2c(F)ccc(F)c21]

Step 1: To a solution of 3-[7-(3, 5-difluoro-2-pyridyl)-4, 7-diazaspiro [2.5]octan-4-yl]-4, 7-difluoro-1H-indazole (30 mg, 79.5 μmol, 1 eq), 1-cyclopropylsulfonylpyrrole-3-sulfonyl chloride (32.2 mg, 119 umol, 1.5 eq) in DCM (1 mL) was added TEA (24.1 mg, 239 umol, 33.2 μL, 3 eq) and DMAP (971 μg, 7.95 μmol, 0.1 eq) at 0° C., and then the mixture was stirred at 20° C. for 0.5 hour under N₂ atmosphere. LC-MS showed 3-[7-(3, 5-difluoro-2-pyridyl)-4, 7-diazaspiro [2.5]octan-4-yl]-4, 7-difluoro-1H-indazole was consumed completely and one main peak with desired mass was detected. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-TLC (SiO₂, petroleum ether/EtOAc=3/1) to give desired 1-(1-cyclopropylsulfonylpyrrol-3-yl) sulfonyl-3-[7-(3, 5-difluoro-2-pyridyl)-4, 7-diazaspiro [2.5]octan-4-yl]-4, 7-difluoro-indazole (6 mg, 9.56 μmol, 12.0% yield, 97.30% purity) as a white solid. ¹H NMR (400 MHz, DMSO-d₆) δ 8.11-8.07 (m, 1H), 7.89-7.86 (m, 1H), 7.84-7.75 (m, 1H), 7.59-7.51 (m, 1H), 7.38-7.34 (m, 1H), 7.27-7.20 (m, 1H), 6.44-6.40 (m, 1H), 3.74-3.65 (m, 2H), 3.56-3.44 (m, 2H), 3.30-3.21 (m, 3H), 1.32-1.25 (m, 2H), 1.20-1.13 (m, 2H), 0.89-0.78 (m, 2H), 0.64-0.53 (m, 2H). HPLC: 97.30% (220 nm), 96.57% (215 nm), 100.00% (254 nm). MS (ESI): mass calcd. For C₂₅H₂₂F₄N₆O₄S₂ 610.11, m/z found 611.3 [M+H]⁺.

### Compound 146: 3-[7-(5-chloro-3-fluoro-2-pyridyl)-4, 7-diazaspiro [2.5]octan-4-yl]-1-(1-cyclopropylsulfonylpyrrol-3-yl) sulfonyl-4, 7-difluoro-indazole

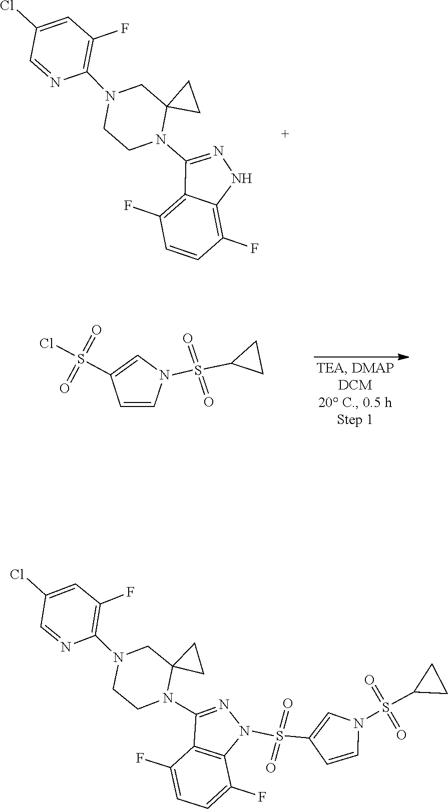 [SMILES: Fc1cc(Cl)cnc1N1CCN(c2n[nH]c3c(F)ccc(F)c23)C2(CC2)C1 ; O=S(=O)(Cl)c1ccn(S(=O)(=O)C2CC2)c1 ; O=S(=O)(c1ccn(S(=O)(=O)C2CC2)c1)n1nc(N2CCN(c3ncc(Cl)cc3F)CC23CC3)c2c(F)ccc(F)c21]

Step 1: To a solution of 3-[7-(5-chloro-3-fluoro-2-pyridyl)-4, 7-diazaspiro [2.5]octan-4-yl]-4, 7-difluoro-1H-indazole (25 mg, 63.5 μmol, 1 eq) and 1-cyclopropylsulfonylpyrrole-3-sulfonyl chloride (25.7 mg, 95.3 μmol, 1.5 eq) in DCM (1 mL) was added TEA (19.3 mg, 190 μmol, 26.5 μL, 3 eq) and DMAP (776 μg, 6.35 μmol, 0.1 eq), and then the mixture was stirred at 20° C. for 0.5 hour. The residue was diluted with H₂O (10 mL) and extracted with EtOAc (5 mL*3). The combined organic layers were washed with brine (10 mL), dried over Na₂SO₄, filtered and concentrated under reduced pressure to give a residue. The residue was purified by prep-TLC (SiO₂, petroleum ether/EtOAc=3/1) to give desired 3-[7-(5-chloro-3-fluoro-2-pyridyl)-4, 7-diazaspiro [2.5]octan-4-yl]-1-(1-cyclopropylsulfonylpyrrol-3-yl) sulfonyl-4, 7-difluoro-indazole (11.5 mg, 17.9 μmol, 28.2% yield, 97.65% purity) as a white solid. ¹H NMR (400 MHz, DMSO-d₆) δ 8.09-8.05 (m, 1H), 7.90-7.86 (m, 1H), 7.85-7.80 (m, 1H), 7.59-7.51 (m, 1H), 7.39-7.35 (m, 1H), 7.28-7.19 (m, 1H), 6.44-6.40 (m, 1H), 3.71-3.65 (m, 2H), 3.63-3.55 (m, 2H), 3.38-3.34 (m, 1H), 3.30-3.23 (m, 2H), 1.32-1.27 (m, 2H), 1.20-1.12 (m, 2H), 0.87-0.81 (m, 2H), 0.62-0.54 (m, 2H). HPLC: 97.65% (220 nm), 97.73% (215 nm), 97.91% (254 nm). MS (ESI): mass calcd. For C₂₅H₂₂ClF₃N₆O₄S₂ 626.08, m/z found 627.0 [M+H]⁺.

### Compound 147: 4-chloro-3-[7-(5-chloro-3-fluoro-2-pyridyl)-4, 7-diazaspiro [2.5]octan-4-yl]-7-fluoro-1-(1-isopropylsulfonylpyrrol-3-yl) sulfonyl-indazole

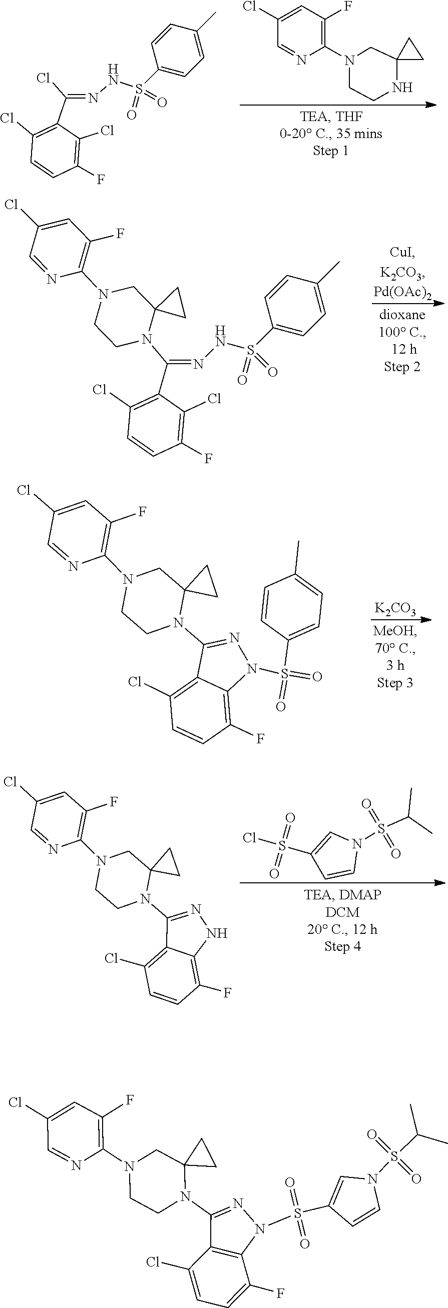

Step 1: To a solution of 7-(5-chloro-3-fluoro-2-pyridyl)-4, 7-diazaspiro [2.5]octane (390 mg, 1.61 mmol, 1 eq) in THF (3 mL) was added dropwise TEA (163 mg, 1.61 mmol, 224 μL, 1 eq) at 20° C. After addition, the mixture was stirred at 20° C. for 5 mins, and then (1Z)-2, 6-dichloro-3-fluoro-N-(p-tolylsulfonyl) benzohydrazonoyl chloride (940 mg, 2.38 mmol, 1.48 eq) in THF (2 mL) was added dropwise at 0° C. The resulting mixture was stirred at 20° C. for 30 mins. LC-MS showed 7-(5-chloro-3-fluoro-2-pyridyl)-4, 7-diazaspiro [2.5]octane was consumed completely and desired mass was detected. The reaction mixture was concentrated under reduced pressure to remove THF. The residue was diluted with H₂O (3 mL) and extracted with DCM (3 mL*2). The combined organic layers were dried over anhydrous Na₂SO₄, filtered and concentrated under reduced pressure to give desired N—[(Z)—[[7-(5-chloro-3-fluoro-2-pyridyl)-4, 7-diazaspiro [2.5]octan-4-yl]-(2, 6-dichloro-3-fluoro-phenyl) methylene]amino]-4-methyl-benzenesulfonamide (1.25 g, crude) as a black oil. MS (ESI): mass calcd. For C₂₅H₂₂C₁₃F₂N₅O₂S 599.05, m/z found 599.9 [M+H]⁺.

Step 2: A mixture of N—[(Z)—[[7-(5-chloro-3-fluoro-2-pyridyl)-4, 7-diazaspiro [2.5]octan-4-yl]-(2, 6-dichloro-3-fluoro-phenyl) methylene]amino]-4-methyl-benzenesulfonamide (1.25 g, 2.08 mmol, 1 eq), CuI (39.6 mg, 208 μmol, 0.1 eq), K₂CO₃ (719 mg, 5.20 mmol, 2.5 eq) and Pd(OAc)₂ (93.4 mg, 416 μmol, 0.2 eq) in dioxane (15 mL) was degassed and purged with N₂ for 3 times, and then the mixture was stirred at 100° C. for 12 hours under N₂ atmosphere. LC-MS showed N—[(Z)—[[7-(5-chloro-3-fluoro-2-pyridyl)-4, 7-diazaspiro [2.5]octan-4-yl]-(2, 6-dichloro-3-fluoro-phenyl) methylene]amino]-4-methyl-benzenesulfonamide was consumed completely and desired mass was detected. The reaction mixture was concentrated under reduced pressure to remove dioxane. The residue was diluted with H₂O (10 mL) and extracted with EtOAc (10 mL*2). The combined organic layers were dried over anhydrous Na₂SO₄, filtered and concentrated under reduced pressure to give desired 4-chloro-3-[7-(5-chloro-3-fluoro-2-pyridyl)-4, 7-diazaspiro [2.5]octan-4-yl]-7-fluoro-1-(p-tolylsulfonyl) indazole (500 mg, crude) as a white solid. MS (ESI): mass calcd. For C₂₅H₂₁Cl₂F₂N₅O₂S 563.08, m/z found 563.9 [M+H]⁺.

Step 3: To a solution of 4-chloro-3-[7-(5-chloro-3-fluoro-2-pyridyl)-4, 7-diazaspiro [2.5]octan-4-yl]-7-fluoro-1-(p-tolylsulfonyl) indazole (500 mg, 886 μmol, 1 eq) and 4-chloro-3-[7-(5-chloro-3-fluoro-2-pyridyl)-4, 7-diazaspiro [2.5]octan-4-yl]-5-fluoro-1-(p-tolylsulfonyl) indazole (500 mg, 886 μmol, 1 eq) in MeOH (10 mL) was added K₂CO₃ (612 mg, 4.43 mmol, 5 eq). The mixture was stirred at 70° C. for 3 hours. LC-MS showed 4-chloro-3-[7-(5-chloro-3-fluoro-2-pyridyl)-4, 7-diazaspiro [2.5]octan-4-yl]-7-fluoro-1-(p-tolylsulfonyl) indazole was consumed completely and desired mass was detected. The reaction mixture was concentrated under reduced pressure to remove MeOH. The residue was diluted with H₂O (10 mL) and extracted with EtOAc 20 mL (10 mL*2). The combined organic layers were dried over anhydrous Na₂SO₄, filtered and concentrated under reduced pressure to give a residue. The residue was purified by prep-TLC (SiO₂, petroleum ether/Ethyl acetate=3/1) to give desired 4-chloro-3-[7-(5-chloro-3-fluoro-2-pyridyl)-4, 7-diazaspiro [2.5]octan-4-yl]-7-fluoro-1H-indazole (40 mg, 97.5 μmol, 11.0% yield) as a yellow oil. MS (ESI): mass calcd. For C₁₈H₁₅Cl₂F₂N₅ 409.07, m/z found 410.1 [M+H]⁺.

Step 4: To a solution of 4-chloro-3-[7-(5-chloro-3-fluoro-2-pyridyl)-4, 7-diazaspiro [2.5]octan-4-yl]-7-fluoro-1H-indazole (40 mg, 97.5 μmol, 1 eq) and 1-isopropylsulfonylpyrrole-3-sulfonyl chloride (39.7 mg, 146 μmol, 1.5 eq) in DCM (1 mL) was added TEA (19.7 mg, 195 μmol, 27.1 μL, 2 eq) and DMAP (1.19 mg, 9.75 μmol, 0.1 eq). The mixture was stirred at 20° C. for 12 hours. LC-MS showed 4-chloro-3-[7-(5-chloro-3-fluoro-2-pyridyl)-4, 7-diazaspiro [2.5]octan-4-yl]-7-fluoro-1H-indazole was consumed completely and no desired mass was detected. The reaction mixture was concentrated under reduced pressure to remove DCM. The residue was diluted with H₂O (1 mL) and extracted with EtOAc 2 mL (1 mL*2). The combined organic layers were dried over anhydrous Na₂SO₄, filtered and concentrated under reduced pressure to give a residue. The residue was purified by prep-HPLC (neutral condition; column: Phenomenex C18 75*30 mm*3 um; mobile phase: [water (NH₄HCO₃)-ACN]; B %: 55%-95%, 8 min) to give desired 4-chloro-3-[7-(5-chloro-3-fluoro-2-pyridyl)-4, 7-diazaspiro [2.5]octan-4-yl]-7-fluoro-1-(1-isopropylsulfonylpyrrol-3-yl)sulfonyl-indazole (3.3 mg, 5.09 μmol, 5.22% yield, 99.69% purity) as a white solid. ¹H NMR (400 MHz, DMSO-d₆) δ 8.07 (d, J=1.9 Hz, 1H), 7.85-7.79 (m, 2H), 7.55-7.48 (m, 1H), 7.46-7.41 (m, 1H), 7.37-7.33 (m, 1H), 6.49 (dd, J=1.6, 3.4 Hz, 1H), 3.99-3.90 (m, 1H), 3.57 (br t, J=4.6 Hz, 4H), 3.48-3.35 (m, 2H), 1.08 (d, J=6.8 Hz, 6H), 0.75 (br s, 2H), 0.47 (br s, 2H). HPLC: 99.69% (220 nm), 99.74% (215 nm), 99.66% (254 nm). MS (ESI): mass calcd. For C₂₅H₂₄Cl₂F₂N₆O₄S₂ 644.06, m/z found 645.1 [M+H]⁺.

### Compound 148: 3-[7-(5-chloro-3-fluoro-2-pyridyl)-4, 7-diazaspiro [2.5]octan-4-yl]-4, 7-difluoro-1-(1-isopropylsulfonylpyrrol-3-yl) sulfonyl-indazole

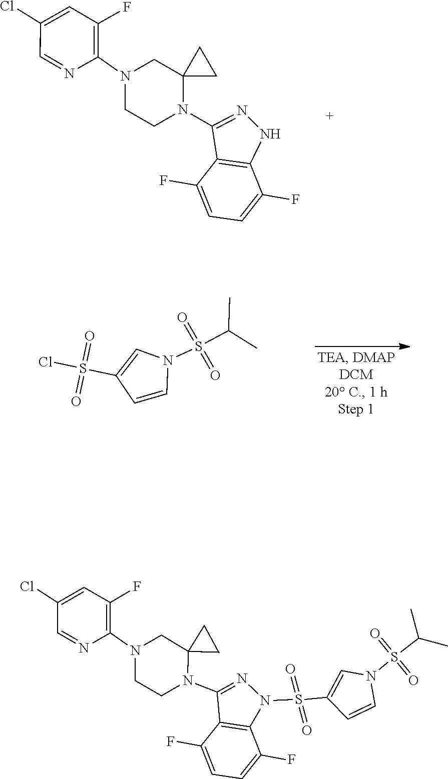 [SMILES: Fc1cc(Cl)cnc1N1CCN(c2n[nH]c3c(F)ccc(F)c23)C2(CC2)C1 ; CC(C)S(=O)(=O)n1ccc(S(=O)(=O)Cl)c1 ; CC(C)S(=O)(=O)n1ccc(S(=O)(=O)n2nc(N3CCN(c4ncc(Cl)cc4F)CC34CC4)c3c(F)ccc(F)c32)c1]

Step 1: To a solution of 3-[7-(5-chloro-3-fluoro-2-pyridyl)-4, 7-diazaspiro [2.5]octan-4-yl]-4, 7-difluoro-1H-indazole (50 mg, 127 μmol, 1 eq) in DCM (2 mL) was added 1-isopropylsulfonylpyrrole-3-sulfonyl chloride (51.7 mg, 190 μmol, 1.5 eq), DMAP (1.55 mg, 12.7 μmol, 0.1 eq) and TEA (38.5 mg, 381 μmol, 53.0 μL, 3 eq). The mixture was stirred at 20° C. for 1 hour. LC-MS showed 3-[7-(5-chloro-3-fluoro-2-pyridyl)-4, 7-diazaspiro [2.5]octan-4-yl]-4, 7-difluoro-1H-indazole was consumed completely and desired mass was detected. The mixture was separated between 20 mL of H₂O and 10 mL of ethyl acetate. The organic phase was separated, washed with 20 mL of brine, dried over Na₂SO₄, filtered and concentrated under reduced pressure to give the crude product. The residue was purified by prep-HPLC (neutral condition; column: Waters Xbridge Prep OBD C18 150*40 mm*10 um; mobile phase: [water(NH₄HCO₃)-ACN]; B %: 65%-95%, 8 min) to give desired 3-[7-(5-chloro-3-fluoro-2-pyridyl)-4, 7-diazaspiro [2.5]octan-4-yl]-4, 7-difluoro-1-(1-isopropylsulfonylpyrrol-3-yl) sulfonyl-indazole (22.6 mg, 35.9 μmol, 45.2% yield, 99.89% purity) as an white solid. ¹H NMR (400 MHz, DMSO-d₆) δ 8.09 (d, J=1.8 Hz, 1H), 7.85 (dd, J=2.1, 12.9 Hz, 1H), 7.80 (t, J=1.9 Hz, 1H), 7.61-7.53 (m, 1H), 7.39-7.34 (m, 1H), 7.25 (dt, J=2.4, 9.2 Hz, 1H), 6.49 (dd, J=1.6, 3.3 Hz, 1H), 4.02-3.93 (m, 1H), 3.70 (br t, J=4.7 Hz, 2H), 3.61 (br s, 2H), 3.34 (br s, 2H), 1.10 (d, J=6.8 Hz, 6H), 0.87 (br s, 2H), 0.62 (br s, 2H). HPLC: 99.89% (220 nm), 100.00% (215 nm), 100.00% (254 nm). MS (ESI): mass calcd. For C₂₅H₂₄ClF₃N₆O₄S₂ 628.09, m/z found 629.3 [M+H]⁺.

### Compound 149: 4, 7-difluoro-3-(7-(5-fluoropyrimidin-2-yl)-4, 7-diazaspiro [2.5]octan-4-yl)-1-((1-(isopropylsulfonyl)-1H-pyrrol-3-yl) sulfonyl)-1H-indazole

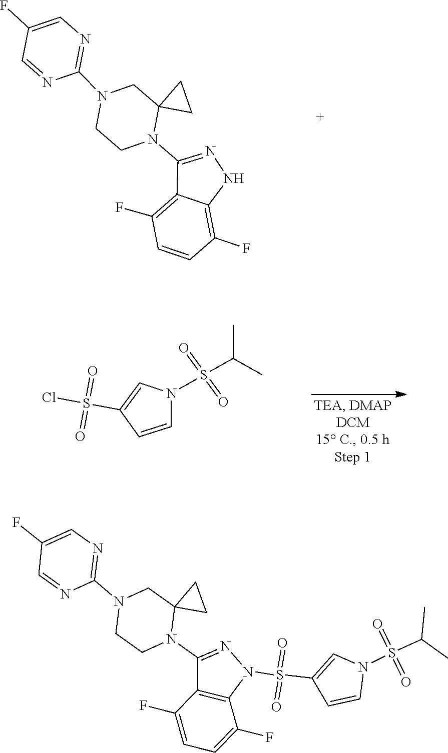 [SMILES: Fc1cnc(N2CCN(c3n[nH]c4c(F)ccc(F)c34)C3(CC3)C2)nc1 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cn4)CC34CC4)c3c(F)ccc(F)c32)c1]

Step 1: To a solution of 4, 7-difluoro-3-[7-(5-fluoropyrimidin-2-yl)-4, 7-diazaspiro [2.5]octan-4-yl]-1H-indazole (50 mg, 139 μmol, 1 eq) and 1-isopropylsulfonylpyrrole-3-sulfonyl chloride (56.6 mg, 208 μmol, 1.5 eq) in DCM (1 mL) was added TEA (28.1 mg, 278 μmol, 38.6 μL, 2 eq) and DMAP (1.70 mg, 13.9 μmol, 0.1 eq). The mixture was stirred at 15° C. for 0.5 hour. LC-MS showed 4, 7-difluoro-3-[7-(5-fluoropyrimidin-2-yl)-4, 7-diazaspiro [2.5]octan-4-yl]-1H-indazole was consumed completely and desired mass was detected. The crude was added H₂O (10 mL), and extracted with EtOAc (15 mL*3). The combined organic layers were washed with brine (10 mL), dried over Na₂SO₄, filtered and concentrated under reduced pressure to give a residue. The residue was purified by prep-HPLC (neutral condition; column: column: Phenomenex C18 75*30 mm*3 um; mobile phase: [water(NH₄HCO₃)-ACN]; B %: 25%-60%, 8 min) to give desired 4, 7-difluoro-3-(7-(5-fluoropyrimidin-2-yl)-4, 7-diazaspiro [2.5]octan-4-yl)-1-((1-(isopropylsulfonyl)-1H-pyrrol-3-yl) sulfonyl)-1H-indazole (6.6 mg, 10.77 μmol, 7.76% yield, 97.17% purity) as a white solid. ¹H NMR (400 MHz, DMSO-d₆) δ 8.46 (s, 2H), 7.79 (t, J=1.8 Hz, 1H), 7.60-7.52 (m, 1H), 7.36 (s, 1H), 7.28-7.20 (m, 1H), 6.49 (dd, J=1.6, 3.3 Hz, 1H), 4.00-3.93 (m, 1H), 3.89 (s, 2H), 3.62 (br s, 4H), 1.10 (d, J=6.8 Hz, 6H), 0.86 (s, 2H), 0.61 (s, 2H). HPLC: 97.17% (220 nm), 97.27% (215 nm), 97.92 (254 nm). MS (ESI): mass calcd. For C₂₄H₂₄F₃N₇O₄S₂ 595.13 m/z found 596.2 [M+H]⁺.

### Compound 150: 4-chloro-3-(7-(5-chloro-3-fluoropyridin-2-yl)-4, 7-diazaspiro [2.5]octan-4-yl)-6-fluoro-1-((1-(isopropylsulfonyl)-1H-pyrrol-3-yl) sulfonyl)-1H-indazole

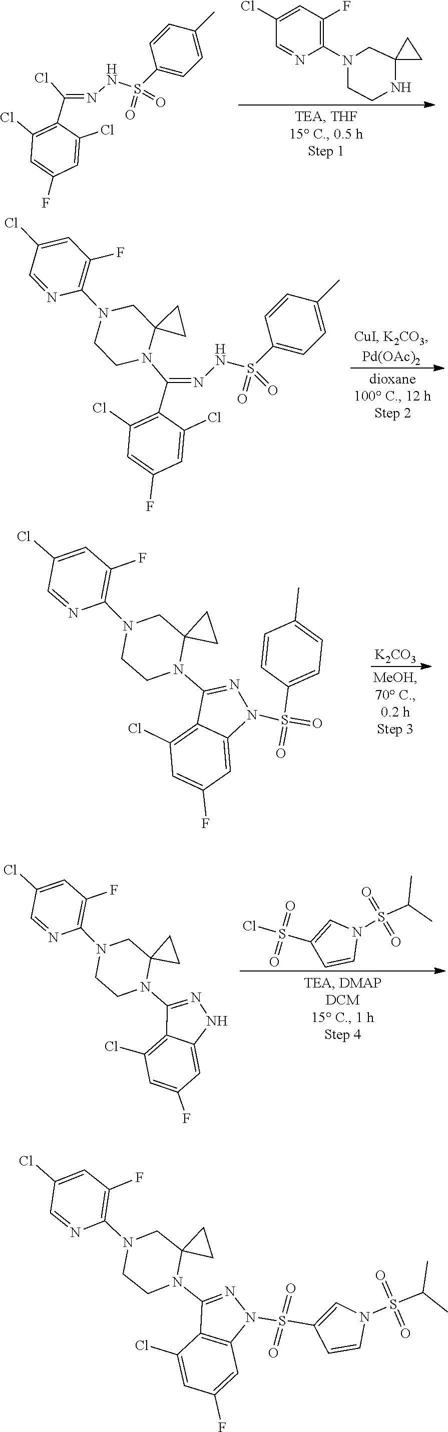

Step 1: To a solution of (1Z)-2, 6-dichloro-4-fluoro-N-(p-tolylsulfonyl)benzohydrazonoyl chloride (1.14 g, 2.88 mmol, 1 eq) and 7-(5-chloro-3-fluoro-2-pyridyl)-4, 7-diazaspiro [2.5]octane (800 mg, 2.88 mmol, 1 eq, HCl) in THF (10 mL) was added TEA (582 mg, 5.75 mmol, 801 μL, 2 eq). The mixture was stirred at 15° C. for 0.5 hour. LC-MS showed 2, 6-dichloro-4-fluoro-N′-(p-tolylsulfonyl) benzohydrazide was consumed completely and desired mass was detected. The crude was added H₂O (20 mL) and extracted with EtOAc (15 mL*3). The combined organic layers were washed with brine (20 mL), dried over anhydrous Na₂SO₄, filtered and concentrated under reduced pressure to give desired (Z)—N′-((7-(5-chloro-3-fluoropyridin-2-yl)-4, 7-diazaspiro [2.5]octan-4-yl) (2, 6-dichloro-4-fluorophenyl) methylene)-4-methylbenzenesulfonohydrazide (1.7 g, crude) as a yellow solid. MS (ESI): mass calcd. For C₂₅H₂₂C₁₃F₂N₅O₂S 599.05, m/z found 600.1 [M+H]⁺.

Step 2: A mixture of N—[(Z)—[[7-(5-chloro-3-fluoro-2-pyridyl)-4, 7-diazaspiro [2.5]octan-4-yl]-(2, 6-dichloro-4-fluoro-phenyl) methylene]amino]-4-methyl-benzenesulfonamide (1.7 g, 2.83 mmol, 1 eq), CuI (53.9 mg, 283 μmol, 0.1 eq), K₂CO₃ (978 mg, 7.07 mmol, 2.5 eq) and Pd(OAc)₂ (127 mg, 566 μmol, 0.2 eq) in dioxane (20 mL) was degassed and purged with N₂ for 3 times, and then the mixture was stirred at 100° C. for 12 hours under N₂ atmosphere. LC-MS showed N—[(Z)—[[7-(5-chloro-3-fluoro-2-pyridyl)-4, 7-diazaspiro [2.5]octan-4-yl]-(2, 6-dichloro-4-fluoro-phenyl) methylene]amino]-4-methyl-benzenesulfonamide was consumed completely and desired mass was detected. The crude was added H₂O (40 mL) and extracted with EtOAc (30 mL*3). The combined organic layers were washed with brine (40 mL), dried over anhydrous Na₂SO₄, filtered and concentrated under reduced pressure to give desired 4-chloro-3-(7-(5-chloro-3-fluoropyridin-2-yl)-4, 7-diazaspiro [2.5]octan-4-yl)-6-fluoro-1-tosyl-1H-indazole (1.6 g, crude) as a yellow solid. MS (ESI): mass calcd. For C₂₅H₂₁Cl₂F₂N₅O₂S 563.08, m/z found 564.1 [M+H]⁺.

Step 3: To a solution of 4-chloro-3-[7-(5-chloro-3-fluoro-2-pyridyl)-4, 7-diazaspiro [2.5]octan-4-yl]-6-fluoro-1-(p-tolylsulfonyl) indazole (1.6 g, 2.83 mmol, 1 eq) in MeOH (20 mL) was added K₂CO₃ (1.96 g, 14.2 mmol, 5 eq). The mixture was stirred at 70° C. for 0.2 hour. LC-MS showed 4-chloro-3-[7-(5-chloro-3-fluoro-2-pyridyl)-4, 7-diazaspiro [2.5]octan-4-yl]-6-fluoro-1-(p-tolylsulfonyl) indazole was consumed completely and desired mass was detected. The reaction mixture was concentrated under reduced pressure to give a residue. The crude was added H₂O (40 mL) and extracted with EtOAc (30 mL*3). The combined organic layers were washed with brine (40 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜35% EtOAc/petroleum ether gradient @100 mL/min) to give desired 4-chloro-3-(7-(5-chloro-3-fluoropyridin-2-yl)-4, 7-diazaspiro [2.5]octan-4-yl)-6-fluoro-1H-indazole (300 mg, 731 μmol, 25.8% yield) as a yellow oil. MS (ESI): mass calcd. For C₁₈H₁₅Cl₂F₂N₅ 409.07, m/z found 410.0 [M+H]⁺.

Step 4: To a solution of 4-chloro-3-[7-(5-chloro-3-fluoro-2-pyridyl)-4, 7-diazaspiro [2.5]octan-4-yl]-6-fluoro-1H-indazole (50 mg, 122 μmol, 1 eq) and 1-isopropylsulfonylpyrrole-3-sulfonyl chloride (43.1 mg, 158 μmol, 1.3 eq) in DCM (1 mL) was added TEA (24.7 mg, 244 μmol, 33.9 μL, 2 eq) and DMAP (1.49 mg, 12.19 μmol, 0.1 eq). The mixture was stirred at 15° C. for 1 hour. LC-MS showed 4-chloro-3-[7-(5-chloro-3-fluoro-2-pyridyl)-4, 7-diazaspiro [2.5]octan-4-yl]-6-fluoro-1H-indazole was consumed completely and desired mass was detected. The crude was added H₂O (10 mL), and extracted with EtOAc (15 mL*3). The combined organic layers were washed with brine 10 mL, dried over Na₂SO₄, filtered and concentrated under reduced pressure to give a residue. The residue was purified by prep-TLC (petroleum ether/Ethyl acetate=3/1) to give desired 4-chloro-3-(7-(5-chloro-3-fluoropyridin-2-yl)-4, 7-diazaspiro [2.5]octan-4-yl)-6-fluoro-1-((1-(isopropylsulfonyl)-1H-pyrrol-3-yl) sulfonyl)-1H-indazole (4.4 mg, 6.50 μmol, 5.33% yield, 95.30% purity) as a white solid. ¹H NMR (400 MHz, DMSO-d₆) δ 8.09 (d, J=1.6 Hz, 1H), 8.05 (t, J=1.9 Hz, 1H), 7.82 (br d, J=2.3 Hz, 2H), 7.54 (dd, J=1.9, 9.1 Hz, 1H), 7.37-7.33 (m, 1H), 6.59 (dd, J=1.6, 3.3 Hz, 1H), 3.99-3.90 (m, 1H), 3.68-3.57 (m, 4H), 3.30 (s, 2H), 1.08 (d, J=6.8 Hz, 6H), 0.79 (br s, 2H), 0.49 (br s, 2H). HPLC: 95.30% (220 nm), 95.15% (215 nm), 96.75 (254 nm). MS (ESI): mass calcd. For C₂₅H₂₄Cl₂F₂N₆O₄S₂ 644.06 m/z found 645.1 [M+H]⁺.

### Compound 151: 4-chloro-3-[7-(5-chloro-3-fluoro-2-pyridyl)-4, 7-diazaspiro [2.5]octan-4-yl]-1-(1-isopropylsulfonylpyrrol-3-yl) sulfonyl-pyrazolo [3, 4-c]pyridine

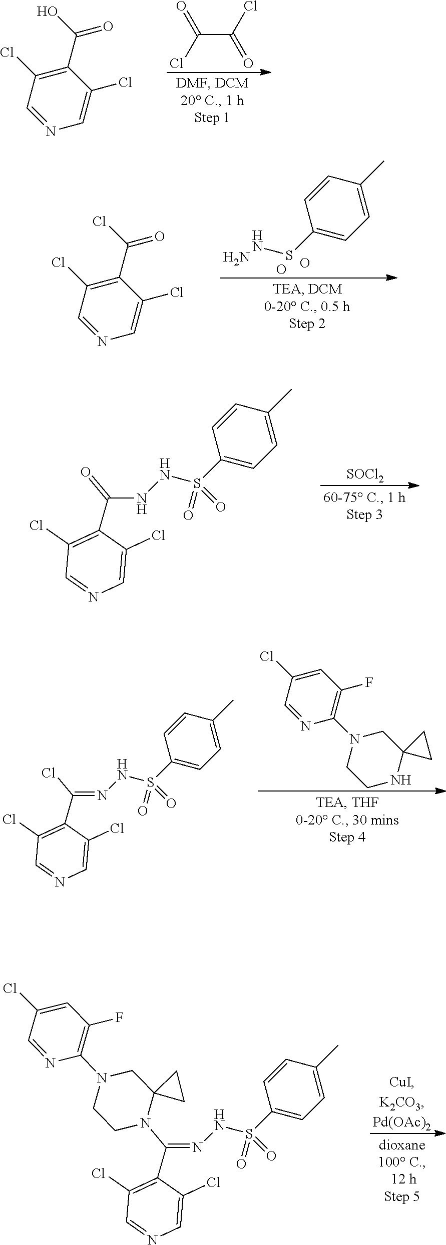

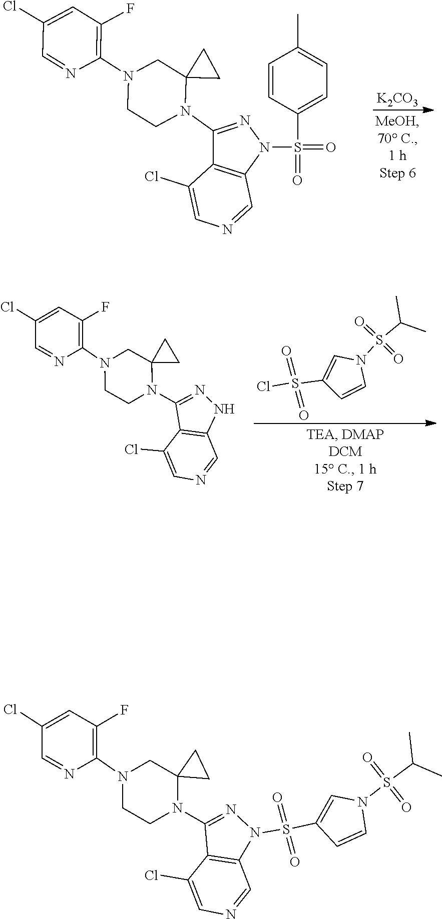

Step 1: To a solution of 3, 5-dichloropyridine-4-carboxylic acid (4 g, 20.8 mmol, 1 eq) in DCM (40 mL) was added oxalyl dichloride (7.93 g, 62.5 mmol, 5.47 mL, 3 eq) and DMF (76.1 mg, 1.04 mmol, 80.2 μL, 0.05 eq). The mixture was stirred at 20° C. for 1 hour. The reaction mixture was quenched by addition MeOH (0.5 mL). TLC (petroleum ether/Ethyl acetate=2/1) indicated 3, 5-dichloropyridine-4-carboxylic acid was consumed completely and one new spot formed. The reaction was clean according to TLC. The reaction mixture was concentrated under reduced pressure to remove DCM to give desired 3, 5-dichloropyridine-4-carbonyl chloride (4.73 g, crude) as a yellow solid.

Step 2: To a solution of 4-methylbenzenesulfonohydrazide (1.59 g, 8.55 mmol, 1.5 eq) in DCM (10 mL) was added TEA (1.44 g, 14.3 mmol, 1.98 mL, 2.5 eq). Then 3, 5-dichloropyridine-4-carbonyl chloride (1.2 g, 5.70 mmol, 1 eq) in DCM (5 mL) was added dropwise at 0° C. The mixture was stirred at 20° C. for 0.5 hour. LC-MS showed 4-methylbenzenesulfonohydrazide was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired 3, 5-dichloro-N′-(p-tolylsulfonyl)pyridine-4-carbohydrazide (1.95 g, crude) as a white solid. MS (ESI): mass calcd. For C₁₃H₁₁Cl₂N₃O₃S 358.99, m/z found 360.1 [M+H]⁺.

Step 3: To a solution of 3, 5-dichloro-N′-(p-tolylsulfonyl) pyridine-4-carbohydrazide (250 mg, 694 μmol, 1 eq) and SOCl₂ (5 mL) was stirred at 75° C. for 0.5 hour. The reaction was cooled to 60° C. and an additional portion of 3, 5-dichloro-N′-(p-tolylsulfonyl) pyridine-4-carbohydrazide (250 mg, 694 μmol, 1 eq) was added and the reaction heated back to 75° C. for 0.5 hour. LC-MS showed 3, 5-dichloro-N′-(p-tolylsulfonyl) pyridine-4-carbohydrazide was consumed completely and desired mass was detected. The reaction mixture was filtered and the filtrate was concentrated under reduced pressure to give desired (4Z)-3, 5-dichloro-N-(p-tolylsulfonyl) pyridine-4-carbohydrazonoyl chloride (520 mg, crude) as a yellow solid. MS (ESI): mass calcd. For C₁₃H₁₀Cl₃N₃O₂S 376.96, m/z found 427.1 [M+H+49]⁺.

Step 4: To a solution of 7-(5-chloro-3-fluoro-2-pyridyl)-4, 7-diazaspiro [2.5]octane (206 mg, 739 μmol, 1 eq, HCl) and TEA (150 mg, 1.48 mmol, 206 μL, 2 eq) in THF (5 mL) was added (4Z)-3, 5-dichloro-N-(p-tolylsulfonyl)pyridine-4-carbohydrazonoyl chloride (280 mg, 739 μmol, 1 eq) at 0° C. The mixture was stirred at 20° C. for 0.5 hour. LC-MS showed (4Z)-3, 5-dichloro-N-(p-tolylsulfonyl) pyridine-4-carbohydrazonoyl chloride was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N—[(Z)—[[7-(5-chloro-3-fluoro-2-pyridyl)-4, 7-diazaspiro [2.5]octan-4-yl]-(3, 5-dichloro-4-pyridyl) methylene]amino]-4-methyl-benzenesulfonamide (425 mg, crude) as a white solid. MS (ESI): mass calcd. For C₂₄H₂₂C₁₃FN₆O₂S 582.06, m/z found 583.1 [M+H]⁺.

Step 5: A mixture of N—[(Z)—[[7-(5-chloro-3-fluoro-2-pyridyl)-4, 7-diazaspiro [2.5]octan-4-yl]-(3, 5-dichloro-4-pyridyl) methylene]amino]-4-methyl-benzenesulfonamide (420 mg, 719 μmol, 1 eq), CuI (13.7 mg, 71.9 μmol, 0.1 eq), K₂CO₃ (249 mg, 1.80 mmol, 2.5 eq) and Pd(OAc)₂ (32.3 mg, 144 μmol, 0.2 eq) in dioxane (6 mL) was degassed and purged with N₂ for 3 times, and then the mixture was stirred at 100° C. for 12 hours under N₂ atmosphere. LC-MS showed N—[(Z)-[[7-(5-chloro-3-fluoro-2-pyridyl)-4, 7-diazaspiro [2.5]octan-4-yl]-(3, 5-dichloro-4-pyridyl) methylene]amino]-4-methyl-benzenesulfonamide was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether/Ethyl acetate=2/1) to give desired 4-chloro-3-[7-(5-chloro-3-fluoro-2-pyridyl)-4, 7-diazaspiro [2.5]octan-4-yl]-1-(p-tolylsulfonyl) pyrazolo [3, 4-c]pyridine (73 mg, 133 μmol, 18.5% yield) as a white solid. MS (ESI): mass calcd. For C₂₄H₂₁Cl₂FN₆O₂S 546.08, m/z found 547.1 [M+H]⁺.

Step 6: To a solution of 4-chloro-3-[7-(5-chloro-3-fluoro-2-pyridyl)-4, 7-diazaspiro [2.5]octan-4-yl]-1-(p-tolylsulfonyl) pyrazolo [3, 4-c]pyridine (73 mg, 133 μmol, 1 eq) in MeOH (2 mL) was added K₂CO₃ (92.2 mg, 667 μmol, 5 eq). The mixture was stirred at 70° C. for 1 hour. LC-MS showed 4-chloro-3-[7-(5-chloro-3-fluoro-2-pyridyl)-4, 7-diazaspiro [2.5]octan-4-yl]-1-(p-tolylsulfonyl) pyrazolo [3, 4-c]pyridine was consumed completely and desired mass was detected. The reaction mixture was filtered and the filter liquor was concentrated under reduced pressure to give a residue. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether/Ethyl acetate=1/1) to give desired 4-chloro-3-[7-(5-chloro-3-fluoro-2-pyridyl)-4, 7-diazaspiro [2.5]octan-4-yl]-1H-pyrazolo [3, 4-c]pyridine (23 mg, 58.5 μmol, 43.9% yield) as a white solid. MS (ESI): mass calcd. For C₁₇H₁₅Cl₂FN₆ 392.07, m/z found 393.0 [M+H]⁺.

Step 7: To a solution of 4-chloro-3-[7-(5-chloro-3-fluoro-2-pyridyl)-4, 7-diazaspiro [2.5]octan-4-yl]-1H-pyrazolo [3, 4-c]pyridine (23 mg, 58.5 μmol, 1 eq) and 1-isopropylsulfonylpyrrole-3-sulfonyl chloride (23.8 mg, 87.7 μmol, 1.5 eq) in DCM (1 mL) was added TEA (11.8 mg, 117 μmol, 2 eq) and DMAP (715 μg, 5.85 μmol, 0.1 eq). The mixture was stirred at 15° C. for 1 hour. LC-MS showed 4-chloro-3-[7-(5-chloro-3-fluoro-2-pyridyl)-4, 7-diazaspiro [2.5]octan-4-yl]-1H-pyrazolo [3, 4-c]pyridine was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether/Ethyl acetate=3/1) to give desired 4-chloro-3-[7-(5-chloro-3-fluoro-2-pyridyl)-4, 7-diazaspiro[2.5]octan-4-yl]-1-(1-isopropylsulfonylpyrrol-3-yl)sulfonyl-pyrazolo [3, 4-c]pyridine (6.2 mg, 9.59 μmol, 16.4% yield, 97.25% purity) as a white solid. ¹H NMR (400 MHz, DMSO-d₆) δ 9.31 (s, 1H), 8.57 (s, 1H), 8.09-8.06 (m, 2H), 7.84 (dd, J=2.1, 12.9 Hz, 1H), 7.36 (dd, J=2.4, 3.3 Hz, 1H), 6.60 (dd, J=1.6, 3.4 Hz, 1H), 3.97-3.90 (m, 1H), 3.72-3.62 (m, 5H), 3.44-3.38 (m, 2H), 3.29 (s, 1H), 1.07 (d, J=6.8 Hz, 8H). HPLC: 97.25% (220 nm), 96.66% (215 nm), 98.47% (254 nm). MS (ESI): mass calcd. For C₂₄H₂₄Cl₂FN₇O₄S₂627.07, m/z found 628.0 [M+H]⁺.

### Compound 152: 4-chloro-3-[7-(5-chloro-3-fluoro-2-pyridyl)-4, 7-diazaspiro [2.5]octan-4-yl]-1-(1-isopropylsulfonylpyrrol-3-yl) sulfonyl-pyrazolo [3, 4-b]pyridine

### Compound 153: 4-chloro-3-(7-(5-chloro-3-fluoropyridin-2-yl)-4,7-diazaspiro[2.5]octan-4-yl)-1-((1-(isopropylsulfonyl)-1H-pyrrol-3-yl)sulfonyl)-1H-pyrazolo[4,3-c]pyridine

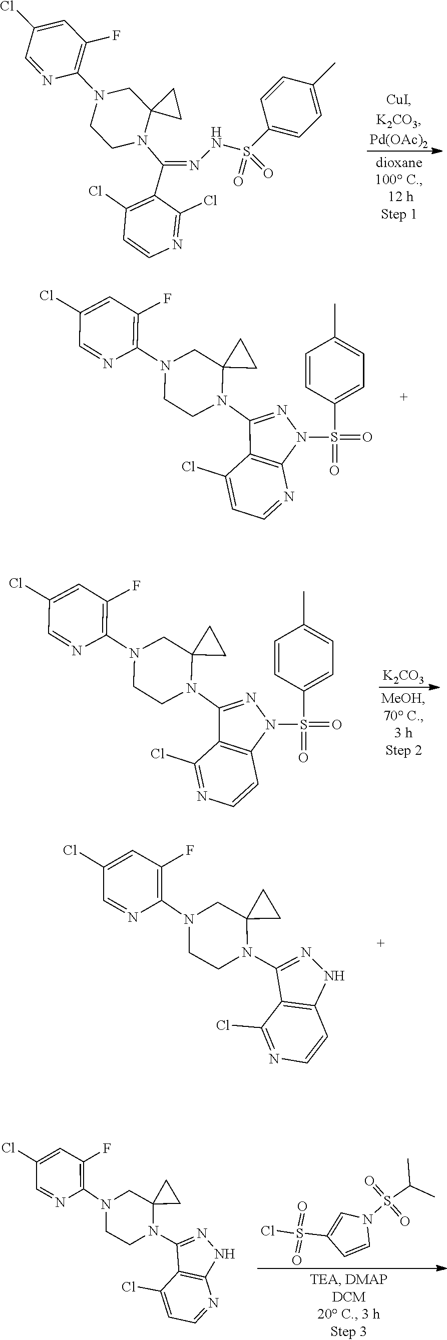

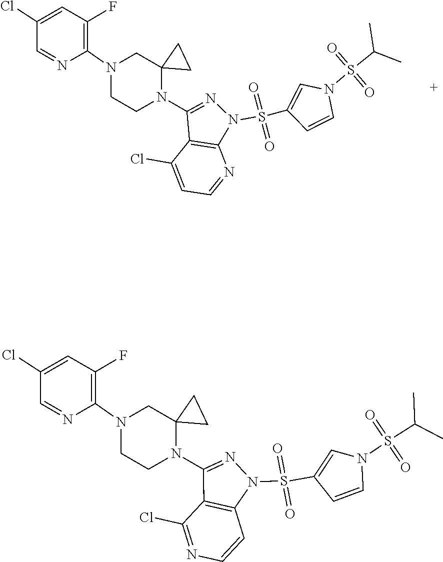 [SMILES: CC(C)S(=O)(=O)n1ccc(S(=O)(=O)n2nc(N3CCN(c4ncc(Cl)cc4F)CC34CC4)c3c(Cl)ccnc32)c1 ; CC(C)S(=O)(=O)n1ccc(S(=O)(=O)n2nc(N3CCN(c4ncc(Cl)cc4F)CC34CC4)c3c(Cl)nccc32)c1]

Step 1: A mixture of N—[(Z)—[[7-(5-chloro-3-fluoro-2-pyridyl)-4, 7-diazaspiro [2.5]octan-4-yl]-(2, 4-dichloro-3-pyridyl) methylene]amino]-4-methyl-benzenesulfonamide (200 mg, 343 mol, 1 eq), CuI (6.52 mg, 34.3 μmol, 0.1 eq), Pd(OAc)₂ (15.4 mg, 68.5 μmol, 0.2 eq) and K₂CO₃ (118 mg, 856 μmol, 2.5 eq) in 1, 4-dioxane (2 mL) was degassed and purged with N₂ for 3 times, and then the mixture was stirred at 100° C. for 12 hours under N₂ atmosphere. LC-MS showed N—[(Z)-[[7-(5-chloro-3-fluoro-2-pyridyl)-4, 7-diazaspiro [2.5]octan-4-yl]-(2, 4-dichloro-3-pyridyl) methylene]amino]-4-methyl-benzenesulfonamide was consumed completely and desired mass was detected. The reaction mixture was diluted with H₂O (5 mL) and extracted with EtOAc (50 mL). The combined organic layers were washed with brine (10 mL), dried over anhydrous Na₂SO₄, filtered and concentrated under reduced pressure to give desired the mixture of 4-chloro-3-[7-(5-chloro-3-fluoro-2-pyridyl)-4, 7-diazaspiro [2.5]octan-4-yl]-1-(p-tolylsulfonyl) pyrazolo [3, 4-b]pyridine and 4-chloro-3-(7-(5-chloro-3-fluoropyridin-2-yl)-4,7-diazaspiro[2.5]octan-4-yl)-1-tosyl-1H-pyrazolo[4,3-c]pyridine (180 mg, crude) as a yellow oil. MS (ESI): mass calcd. For C₂₄H₂₁Cl₂FN₆O₂S 546.08, m/z found 547.0 [M+H]⁺.

Step 2: To a solution of 4-chloro-3-[7-(5-chloro-3-fluoro-2-pyridyl)-4, 7-diazaspiro [2.5]octan-4-yl]-1-(p-tolylsulfonyl) pyrazolo [3, 4-b]pyridine and 4-chloro-3-(7-(5-chloro-3-fluoropyridin-2-yl)-4,7-diazaspiro[2.5]octan-4-yl)-1-tosyl-1H-pyrazolo[4,3-c]pyridine (180 mg, 329 μmol, 1 eq) in MeOH (3 mL) was added K₂CO₃ (227 mg, 1.64 mmol, 5 eq). The mixture was stirred at 70° C. for 3 hours. LC-MS showed the starting material were consumed completely and desired mass was detected. The reaction mixture was concentrated under reduced pressure to remove MeOH. The residue was diluted with H₂O (5 mL) and extracted with DCM (50 mL). The combined organic layers were washed with brine (20 mL), dried over anhydrous Na₂SO₄, filtered and concentrated under reduced pressure to give a residue. The residue was purified by prep-TLC (SiO₂, petroleum ether/EtOAc=2/1) to give two isomers. The structures were assigned randomly. 4-Chloro-3-[7-(5-chloro-3-fluoro-2-pyridyl)-4, 7-diazaspiro [2.5]octan-4-yl]-1H-pyrazolo [3, 4-b]pyridine (14 mg, 35.6 μmol, 10.8% yield) was obtained as a yellow oil. MS (ESI): mass calcd. For C₁₇H₁₅Cl₂FN₆ 392.07, m/z found 393.0 [M+H]⁺. 4-Chloro-3-(7-(5-chloro-3-fluoropyridin-2-yl)-4,7-diazaspiro[2.5]octan-4-yl)-1H-pyrazolo[4,3-c]pyridine (10 mg, 18.3 μmol, 35.71% yield) was obtained as a yellow oil. MS (ESI): mass calcd. For C₁₇H₁₅Cl₂FN₆ 392.07, m/z found 393.0 [M+H]⁺.

Step 3: To a solution of 4-chloro-3-[7-(5-chloro-3-fluoro-2-pyridyl)-4, 7-diazaspiro [2.5]octan-4-yl]-1H-pyrazolo [3, 4-b]pyridine (14 mg, 35.6 μmol, 1 eq) and 1-isopropylsulfonylpyrrole-3-sulfonyl chloride (12.6 mg, 46.3 μmol, 1.3 eq) in DCM (0.5 mL) was added TEA (10.8 mg, 107 μmol, 14.9 μL, 3 eq) and DMAP (435 μg, 3.56 μmol, 0.1 eq). The mixture was stirred at 20° C. for 3 hours. LC-MS showed 4-chloro-3-[7-(5-chloro-3-fluoro-2-pyridyl)-4, 7-diazaspiro [2.5]octan-4-yl]-1H-pyrazolo [3, 4-b]pyridine was consumed completely and one main peak with desired mass was detected. The reaction mixture was diluted with H₂O (2 mL) and extracted with DCM (4 mL). The combined organic layers were washed with brine (4 mL), dried over anhydrous Na₂SO₄, filtered and concentrated under reduced pressure to give a residue. The residue was purified by prep-HPLC (FA condition: column: Phenomenex Luna C18 75*30 mm*3 um; mobile phase: [water(FA)-ACN]; B %: 40%-80%, 8 min) to give desired 4-chloro-3-[7-(5-chloro-3-fluoro-2-pyridyl)-4, 7-diazaspiro [2.5]octan-4-yl]-1-(1-isopropylsulfonylpyrrol-3-yl) sulfonyl-pyrazolo [3, 4-b]pyridine (3.5 mg, 5.28 μmol, 14.8% yield, 94.76% purity) as a pale yellow solid. ¹H NMR (400 MHz, DMSO-d₆) δ 8.60 (d, J=5.13 Hz, 1H) 8.09 (d, J=1.88 Hz, 1H) 7.84-7.88 (m, 2H) 7.57 (d, J=5.13 Hz, 1H) 7.34 (dd, J=3.19, 2.56 Hz, 1H) 6.55 (dd, J=3.31, 1.56 Hz, 1H) 3.91-4.00 (m, 1H) 3.61-3.71 (m, 4H) 3.45 (br d, J=1.25 Hz, 2H) 1.09 (d, J=6.75 Hz, 6H) 0.79-0.86 (m, 2H) 0.56 (s, 2H). HPLC: 94.76% (220 nm), 94.66% (215 nm), 93.61% (254 nm). MS (ESI): mass calcd. For C₂₄H₂₄Cl₂FN₇O₄S₂ 627.07 m/z found 628.1 [M+H]⁺.

To a solution of 4-chloro-3-(7-(5-chloro-3-fluoropyridin-2-yl)-4,7-diazaspiro[2.5]octan-4-yl)-1H-pyrazolo[4,3-c]pyridine (10 mg, 25.4 μmol, 1 eq) and 1-isopropylsulfonylpyrrole-3-sulfonyl chloride (9.67 mg, 35.6 μmol, 1.4 eq) in DCM (0.5 mL) was added TEA (7.72 mg, 76.3 μmol, 10.6 μL, 3 eq) and DMAP (311 μg, 2.54 μmol, 0.1 eq). The mixture was stirred at 20° C. for 1 hour. LC-MS showed 7-chloro-3-[7-(5-chloro-3-fluoro-2-pyridyl)-4, 7-diazaspiro [2.5]octan-4-yl]-1H-pyrazolo [3, 4-c]pyridine was consumed completely and one main peak with desired mass was detected. The reaction mixture was diluted with H₂O (2 mL) and extracted with DCM (4 mL). The combined organic layers were washed with brine (4 mL), dried over anhydrous Na₂SO₄, filtered and concentrated under reduced pressure to give a residue. The residue was purified by prep-HPLC (FA condition: column: Phenomenex Luna C18 75*30 mm*3 um; mobile phase: [water(FA)-ACN]; B %: 55%-85%, 8 min) to give desired 4-chloro-3-(7-(5-chloro-3-fluoropyridin-2-yl)-4,7-diazaspiro[2.5]octan-4-yl)-1-((1-(isopropylsulfonyl)-1H-pyrrol-3-yl)sulfonyl)-1H-pyrazolo[4,3-c]pyridine (2.1 mg, 3.34 μmol, 13.1% yield, 100.00% purity) as a white solid. ¹H NMR (400 MHz, DMSO-d₆) δ 8.42 (d, J=5.88 Hz, 1H) 7.98-8.10 (m, 3H) 7.83 (dd, J=12.82, 2.06 Hz, 1H) 7.35-7.40 (m, 1H) 6.60 (dd, J=3.31, 1.56 Hz, 1H) 3.90-4.00 (m, 1H) 3.59-3.73 (m, 4H) 3.41 (br s, 2H) 1.08 (d, J=6.75 Hz, 6H) 0.81 (s, 2H) 0.53 (s, 2H). HPLC: 99.26% (220 nm), 99.29% (215 nm), 100.00% (254 nm). MS (ESI): mass calcd. For C₂₄H₂₄Cl₂FN₇O₄S₂ 627.07 m/z found 628.1 [M+H]⁺.

Note: Since the structure of 4-chloro-3-[7-(5-chloro-3-fluoro-2-pyridyl)-4, 7-diazaspiro [2.5]octan-4-yl]-1H-pyrazolo [3, 4-b]pyridine and 4-Chloro-3-(7-(5-chloro-3-fluoropyridin-2-yl)-4,7-diazaspiro[2.5]octan-4-yl)-1H-pyrazolo[4,3-c]pyridine were not confirmed by 2D NMR and they are assigned randomly, after treatment with the isopropylsulfonylpyrrole-3-sulfonyl chloride, the final compounds 4-chloro-3-[7-(5-chloro-3-fluoro-2-pyridyl)-4, 7-diazaspiro [2.5]octan-4-yl]-1-(1-isopropylsulfonylpyrrol-3-yl) sulfonyl-pyrazolo [3, 4-b]pyridine and 4-chloro-3-(7-(5-chloro-3-fluoropyridin-2-yl)-4,7-diazaspiro[2.5]octan-4-yl)-1-((1-(isopropylsulfonyl)-1H-pyrrol-3-yl)sulfonyl)-1H-pyrazolo[4,3-c]pyridine were also assigned randomly.

### Compound 154: 4-chloro-3-(7-(5-chloropyrimidin-2-yl)-4, 7-diazaspiro [2.5]octan-4-yl)-6-fluoro-1-((1-(isopropylsulfonyl)-1H-pyrrol-3-yl) sulfonyl)-1H-indazole

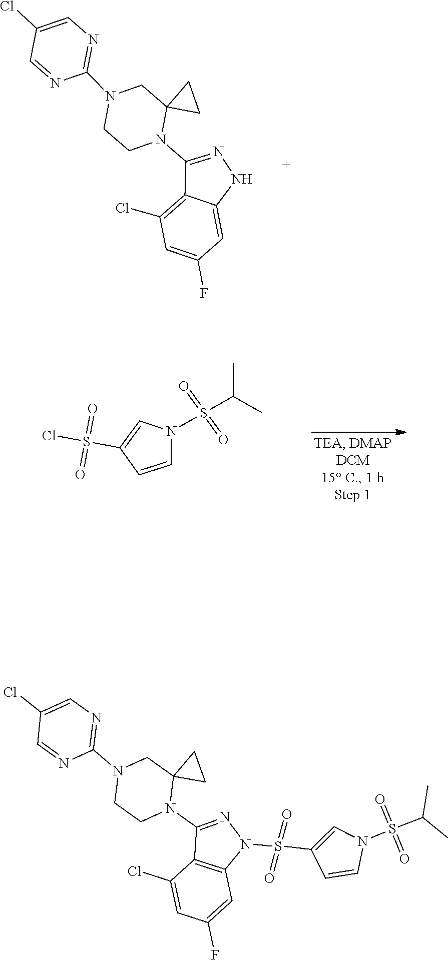 [SMILES: Fc1cc(Cl)c2c(N3CCN(c4ncc(Cl)cn4)CC34CC4)n[nH]c2c1 ; CC(C)S(=O)(=O)n1ccc(S(=O)(=O)Cl)c1 ; CC(C)S(=O)(=O)n1ccc(S(=O)(=O)n2nc(N3CCN(c4ncc(Cl)cn4)CC34CC4)c3c(Cl)cc(F)cc32)c1]

Step 1: To a solution of 4-chloro-3-[7-(5-chloropyrimidin-2-yl)-4, 7-diazaspiro [2.5]octan-4-yl]-6-fluoro-1H-indazole (30 mg, 76.3 μmol, 1 eq) and 1-isopropylsulfonylpyrrole-3-sulfonyl chloride (24.9 mg, 91.5 μmol, 1.2 eq) in DCM (1 mL) was added TEA (15.4 mg, 153 μmol, 21.2 μL, 2 eq) and DMAP (932 μg, 7.63 μmol, 0.1 eq). The mixture was stirred at 15° C. for 1 hour. LC-MS showed 4-chloro-3-[7-(5-chloropyrimidin-2-yl)-4, 7-diazaspiro [2.5]octan-4-yl]-6-fluoro-1H-indazole was consumed completely and desired mass was detected. The reaction mixture was concentrated under reduced pressure to give a residue. The residue was purified by prep-HPLC (column: Phenomenex Luna C18 75*30 mm*3 μm; mobile phase: [water (FA)-ACN]; B %: 60%-80%, 8 min) to give desired 4-chloro-3-(7-(5-chloropyrimidin-2-yl)-4, 7-diazaspiro [2.5]octan-4-yl)-6-fluoro-1-((1-(isopropylsulfonyl)-1H-pyrrol-3-yl) sulfonyl)-1H-indazole (3 mg, 4.59 μmol, 6.02% yield, 96.19% purity) as a white solid. ¹H NMR (400 MHz, DMSO-d₆) δ 8.43 (s, 2H), 8.09-8.00 (m, 1H), 7.84-7.78 (m, 1H), 7.58-7.46 (m, 1H), 7.36 (br s, 1H), 6.59 (br d, J=1.5 Hz, 1H), 3.97 (s, 2H), 3.73 (br s, 3H), 3.58-3.49 (m, 2H), 1.08 (br d, J=6.8 Hz, 6H), 0.80 (br s, 2H), 0.49 (br s, 2H). HPLC: 96.19% (220 nm), 95.62% (215 nm), 98.40 (254 nm). MS (ESI): mass calcd. For C₂₄H₂₄Cl₂FN₇O₄S₂ 627.07 m/z found 628.1 [M+H]⁺.

### Compound 155: 4-chloro-6-fluoro-3-(7-(5-fluoropyrimidin-2-yl)-4, 7-diazaspiro [2.5]octan-4-yl)-1-((1-(isopropylsulfonyl)-1H-pyrrol-3-yl) sulfonyl)-1H-indazole

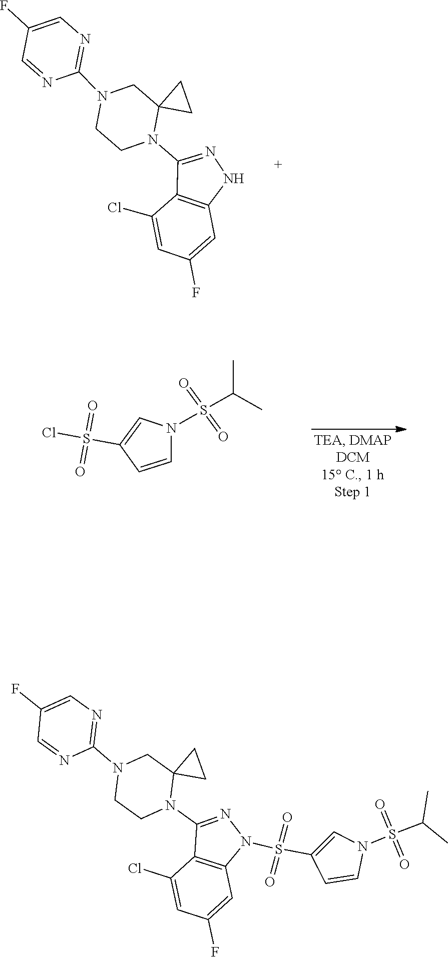 [SMILES: Fc1cnc(N2CCN(c3n[nH]c4cc(F)cc(Cl)c34)C3(CC3)C2)nc1 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cn4)CC34CC4)c3c(Cl)cc(F)cc32)c1]

Step 1: To a solution of 4-chloro-6-fluoro-3-[7-(5-fluoropyrimidin-2-yl)-4,7-diazaspiro[2.5]octan-4-yl]-1H-indazole (60 mg, 159 μmol, 1 eq) and 1-isopropylsulfonylpyrrole-3-sulfonyl chloride (64.9 mg, 239 μmol, 1.5 eq) in DCM (1 mL) was added TEA (32.2 mg, 318 μmol, 44.3 μL, 2 eq) and DMAP (1.95 mg, 15.9 μmol, 0.1 eq). The mixture was stirred at 15° C. for 1 hour. LC-MS showed 4-chloro-6-fluoro-3-[7-(5-fluoropyrimidin-2-yl)-4, 7-diazaspiro [2.5]octan-4-yl]-1H-indazole was consumed completely and desired mass was detected. The reaction mixture was concentrated under reduced pressure to give a residue. The residue was purified by prep-HPLC (neutral condition, column: Phenomenex C18 75*30 mm*3 um; mobile phase: [water (NH₄HCO₃)-ACN]; B %: 60%-80%, 8 mins) to give desired 4-chloro-6-fluoro-3-(7-(5-fluoropyrimidin-2-yl)-4, 7-diazaspiro [2.5]octan-4-yl)-1-((1-(isopropylsulfonyl)-1H-pyrrol-3-yl) sulfonyl)-1H-indazole (23.5 mg, 38.4 μmol, 24.11% yield, 98.38% purity) as a white solid. ¹H NMR (400 MHz, DMSO-d₆) δ 8.47 (s, 2H), 8.08-8.03 (m, 1H), 7.84-7.78 (m, 1H), 7.58-7.51 (m, 1H), 7.39-7.33 (m, 1H), 6.59 (dd, J=1.6, 3.3 Hz, 1H), 3.93 (s, 3H), 3.79-3.62 (m, 2H), 3.54 (br s, 2H), 1.08 (d, J=6.8 Hz, 6H), 0.84-0.76 (m, 2H), 0.52-0.45 (m, 2H). HPLC: 98.38% (220 nm), 98.50% (215 nm), 99.57 (254 nm). MS (ESI): mass calcd. For C₂₄H₂₄ClF₂N₇O₄S₂ 611.10 m/z found 612.2 [M+H]⁺.

### Compound 156: 3-chloro-5-[4-[4-chloro-6-fluoro-1-(1-isopropylsulfonylpyrrol-3-yl) sulfonyl-indazol-3-yl]-4, 7-diazaspiro [2.5]octan-7-yl]-1, 2, 4-thiadiazole

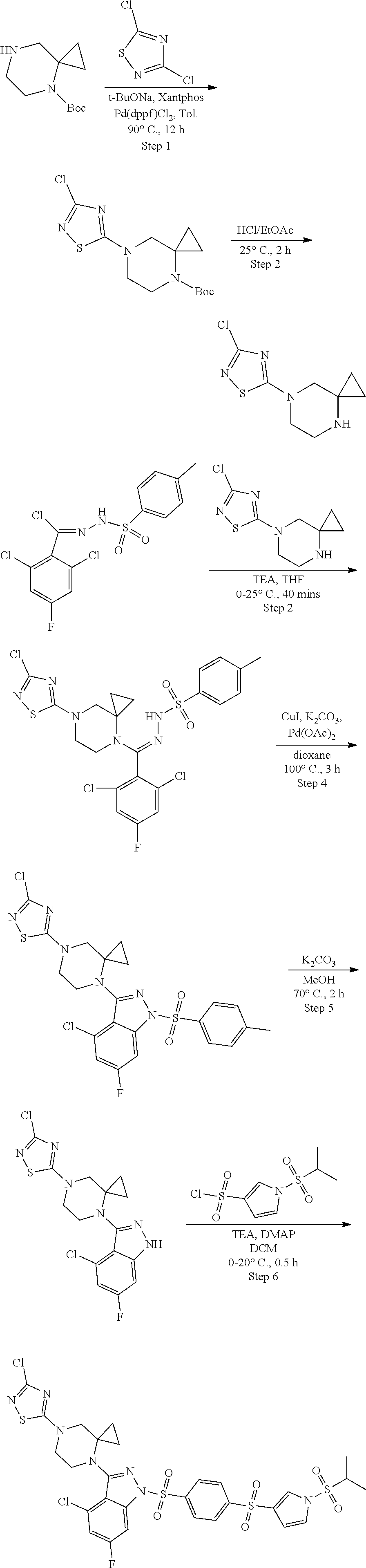

Step 1: A mixture of 3, 5-dichloro-1, 2, 4-thiadiazole (2.3 g, 14.8 mmol, 1.2 eq), tert-butyl 4, 7-diazaspiro [2.5]octane-4-carboxylate (3.08 g, 14.5 mmol, 1.17 eq), Xantphos (1.43 g, 2.47 mmol, 0.2 eq), Pd(dppf)Cl₂ (905 mg, 1.24 mmol, 0.1 eq) and t-BuONa (2.38 g, 24.7 mmol, 2 eq) in Tol. (30 mL) was degassed and purged with N₂ for 3 times, and then the mixture was stirred at 90° C. for 12 hours under N₂ atmosphere. LC-MS showed 3, 5-dichloro-1, 2, 4-thiadiazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/petroleum ether gradient @80 mL/min) to give desired tert-butyl 7-(3-chloro-1, 2, 4-thiadiazol-5-yl)-4, 7-diazaspiro [2.5]octane-4-carboxylate (4 g, crude) as a yellow oil. MS (ESI): mass calcd. For C₁₃H₁₉ClN₄O₂S 330.09, m/z found 331.0 [M+H]⁺.

Step 2: A mixture of tert-butyl 7-(3-chloro-1, 2, 4-thiadiazol-5-yl)-4, 7-diazaspiro [2.5]octane-4-carboxylate (4 g, 12.1 mmol, 1 eq) in HCl/EtOAc (30 mL) was stirred at 25° C. for 2 hours. LC-MS showed tert-butyl 7-(3-chloro-1, 2, 4-thiadiazol-5-yl)-4, 7-diazaspiro [2.5]octane-4-carboxylate was consumed completely and desired mass was detected. The reaction mixture was concentrated to give desired 3-chloro-5-(4, 7-diazaspiro [2.5]octan-7-yl)-1, 2, 4-thiadiazole (2.27 g, crude) was obtained as a white solid. MS (ESI): mass calcd. For C₈H₁₁ClN₄S 230.04, m/z found 231.0 [M+H]⁺.

Step 3: To a solution of 3-chloro-5-(4, 7-diazaspiro [2.5]octan-7-yl)-1, 2, 4-thiadiazole (530 mg, 2.30 mmol, 1 eq) in THF (10 mL) was added dropwise TEA (930 mg, 9.19 mmol, 1.28 mL, 4 eq) at 25° C. After addition, the mixture was stirred at this temperature for 10 mins, and then (1Z)-2, 6-dichloro-4-fluoro-N-(p-tolylsulfonyl) benzohydrazonoyl chloride (1 g, 2.53 mmol, 1.1 eq) in THF (10 mL) was added dropwise at 0° C. The resulting mixture was stirred at 25° C. for 0.5 hour. LC-MS showed 3-chloro-5-(4, 7-diazaspiro [2.5]octan-7-yl)-1, 2, 4-thiadiazole was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(Z)-[[7-(3-chloro-1, 2, 4-thiadiazol-5-yl)-4, 7-diazaspiro [2.5]octan-4-yl]-(2, 6-dichloro-4-fluoro-phenyl) methylene]amino]-4-methyl-benzenesulfonamide (1.4 g, crude) as a yellow solid. MS (ESI): mass calcd. For C₂₂H₂₀Cl₃FN₆O₂S₂ 588.01, m/z found 589.1 [M+H]⁺.

Step 4: A mixture of N—[(Z)—[[7-(3-chloro-1, 2, 4-thiadiazol-5-yl)-4, 7-diazaspiro [2.5]octan-4-yl]-(2, 6-dichloro-4-fluoro-phenyl) methylene]amino]-4-methyl-benzenesulfonamide (1.4 g, 2.37 mmol, 1 eq), CuI (45.2 mg, 237 μmol, 0.1 eq), Pd(OAc)₂ (107 mg, 475 μmol, 0.2 eq) and K₂CO₃ (820 mg, 5.93 mmol, 2.5 eq) in dioxane (20 mL) was degassed and purged with N₂ for 3 times, and then the mixture was stirred at 100° C. for 3 hours under N₂ atmosphere. LC-MS showed N—[(Z)—[[7-(3-chloro-1, 2, 4-thiadiazol-5-yl)-4, 7-diazaspiro [2.5]octan-4-yl]-(2, 6-dichloro-4-fluoro-phenyl) methylene]amino]-4-methyl-benzenesulfonamide was consumed completely and desired compound was detected. Then it was separated between 50 mL of water and 100 mL of ethyl acetate. The organic phase was separated, washed with 50 mL of brine, dried over Na₂SO₄, filtered and concentrated under reduced pressure to give desired 3-chloro-5-[4-[4-chloro-6-fluoro-1-(p-tolylsulfonyl) indazol-3-yl]-4, 7-diazaspiro [2.5]octan-7-yl]-1, 2, 4-thiadiazole (1.4 g, crude) as a black oil. MS (ESI): mass calcd. For C₂₂H₁₉Cl₂FN₆O₂S₂ 552.04, m/z found 553.0 [M+H]⁺.

Step 5: To a solution of 3-chloro-5-[4-[4-chloro-6-fluoro-1-(p-tolylsulfonyl) indazol-3-yl]-4, 7-diazaspiro [2.5]octan-7-yl]-1, 2, 4-thiadiazole (1.4 g, 2.53 mmol, 1 eq) in MeOH (20 mL) was added K₂CO₃ (1.75 g, 12.7 mmol, 5 eq). The mixture was stirred at 70° C. for 2 hours. LC-MS showed 3-chloro-5-[4-[4-chloro-6-fluoro-1-(p-tolylsulfonyl) indazol-3-yl]-4, 7-diazaspiro [2.5]octan-7-yl]-1, 2, 4-thiadiazole was consumed completely and desired compound was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (SiO₂, petroleum ether/Ethyl acetate=2/1) to give desired 3-chloro-5-[4-(4-chloro-6-fluoro-1H-indazol-3-yl)-4, 7-diazaspiro [2.5]octan-7-yl]-1, 2, 4-thiadiazole (330 mg, 827 μmol, 32.7% yield) as a yellow oil. MS (ESI): mass calcd. For C₁₅H₁₃Cl₂FN₆S 398.03, m/z found 399.1 [M+H]⁺.

Step 6: To a solution of 1-isopropylsulfonylpyrrole-3-sulfonyl chloride (68.1 mg, 250 mol, 2 eq) and 3-chloro-5-[4-(4-chloro-6-fluoro-1H-indazol-3-yl)-4, 7-diazaspiro [2.5]octan-7-yl]-1, 2, 4-thiadiazole (50 mg, 125 μmol, 1 eq) in DCM (1 mL) was added TEA (25.3 mg, 250 μmol, 35.0 μL, 2 eq) and DMAP (1.53 mg, 12.5 μmol, 0.1 eq) at 0° C. The mixture was stirred at 20° C. for 0.5 hour. LC-MS showed 3-chloro-5-[4-(4-chloro-6-fluoro-1H-indazol-3-yl)-4, 7-diazaspiro [2.5]octan-7-yl]-1, 2, 4-thiadiazole was consumed completely and desired mass was detected. The reaction mixture was concentrated to give the crude product. The residue was purified by prep-HPLC (basic condition; column: Waters Xbridge Prep OBD C18 150*40 mm*10 um; mobile phase: [water(NH₃H₂O+NH₄HCO₃)-ACN]; B %: 45%-75%, 8 min) to give desired 3-chloro-5-[4-[4-chloro-6-fluoro-1-(1-isopropylsulfonylpyrrol-3-yl) sulfonyl-indazol-3-yl]-4, 7-diazaspiro [2.5]octan-7-yl]-1, 2, 4-thiadiazole (15 mg, 23.6 μmol, 18.8% yield, 99.82% purity) as a white solid. ¹H NMR (400 MHz, DMSO-d₆) δ 8.09 (t, J=1.9 Hz, 1H), 7.82 (dd, J=1.9, 8.7 Hz, 1H), 7.56 (dd, J=1.9, 9.2 Hz, 1H), 7.37 (t, J=2.9 Hz, 1H), 6.61 (dd, J=1.5, 3.3 Hz, 1H), 3.97-3.93 (m, 1H), 3.81-3.73 (m, 2H), 3.63 (br d, J=4.4 Hz, 2H), 3.52-3.46 (m, 2H), 1.09 (d, J=6.9 Hz, 6H), 0.90 (s, 2H), 0.54 (br s, 2H) HPLC: 99.82% (220 nm), 99.79% (215 nm), 100.00% (254 nm). MS (ESI): mass calcd. For C₂₂H₂₂Cl₂FN₇O₄S₃ 633.03, m/z found 634.1 [M+H]⁺.

### Compound 157: 5-chloro-2-(4-(4-chloro-6-fluoro-1-((1-(isopropylsulfonyl)-1H-pyrrol-3-yl) sulfonyl)-1H-indazol-3-yl)-4, 7-diazaspiro [2.5]octan-7-yl) thiazole

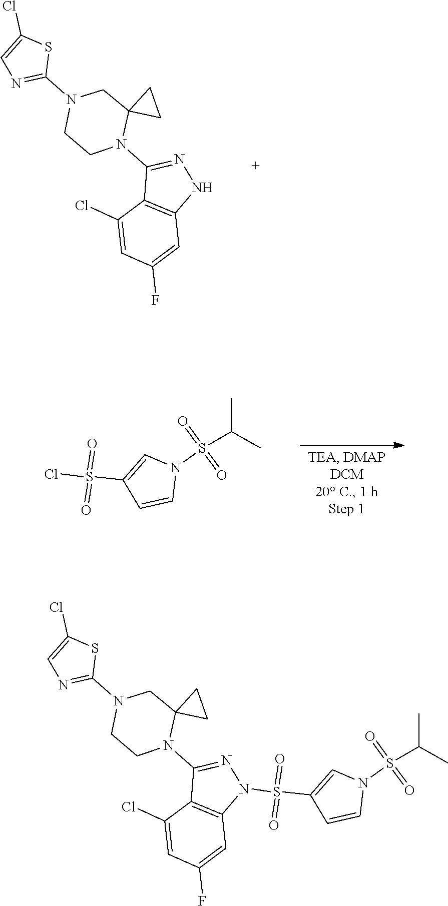 [SMILES: Fc1cc(Cl)c2c(N3CCN(c4ncc(Cl)s4)CC34CC4)n[nH]c2c1 ; CC(C)S(=O)(=O)n1ccc(S(=O)(=O)Cl)c1 ; CC(C)S(=O)(=O)n1ccc(S(=O)(=O)n2nc(N3CCN(c4ncc(Cl)s4)CC34CC4)c3c(Cl)cc(F)cc32)c1]

Step 1: To the solution of 5-chloro-2-[4-(4-chloro-6-fluoro-1H-indazol-3-yl)-4, 7-diazaspiro [2.5]octan-7-yl]thiazole (30 mg, 75.3 μmol, 1 eq), TEA (22.9 mg, 226 μmol, 31.5 μL, 3 eq) and DMAP (920 μg, 7.53 μmol, 0.1 eq) in DCM (2 mL) was added 1-isopropylsulfonylpyrrole-3-sulfonyl chloride (20.5 mg, 75.3 μmol, 1 eq) at 20° C. and the solution was stirred at 20° C. for 1 hour. LCMS showed 5-chloro-2-[4-(4-chloro-6-fluoro-1H-indazol-3-yl)-4, 7-diazaspiro [2.5]octan-7-yl]thiazole was consumed completely and one main peak with desired mass was detected. The residue was purified by prep-TLC (SiO₂, petroleum ether/ethyl acetate=3/1) to give desired 5-chloro-2-(4-(4-chloro-6-fluoro-1-((1-(isopropylsulfonyl)-1H-pyrrol-3-yl) sulfonyl)-1H-indazol-3-yl)-4, 7-diazaspiro [2.5]octan-7-yl) thiazole (17.0 mg, 26.5 μmol, 35.2% yield, 98.88% purity) as a light yellow oil. ¹H NMR (400 MHz, DMSO-d₆) δ 8.06 (t, J=1.8 Hz, 1H), 7.80 (dd, J=2.0, 8.8 Hz, 1H), 7.54 (dd, J=2.0, 9.0 Hz, 1H), 7.38-7.32 (m, 1H), 7.17 (s, 1H), 6.58 (dd, J=1.6, 3.2 Hz, 1H), 3.99-3.89 (m, 1H), 3.67-3.55 (m, 4H), 3.37 (br s, 2H), 1.07 (d, J=6.8 Hz, 6H), 0.85 (s, 2H), 0.51 (br s, 2H). HPLC: 98.88% (220 nm), 98.66% (215 nm), 99.89% (254 nm). MS (ESI): mass calcd. For C₂₃H₂₃Cl₂FN₆O₄S₃ 632.03, m/z found 633.1 [M+H]⁺.

### Compound 158: 4-chloro-3-[7-(3, 5-difluoro-2-pyridyl)-4, 7-diazaspiro [2.5]octan-4-yl]-6-fluoro-1-(1-isopropylsulfonylpyrrol-3-yl) sulfonyl-indazole

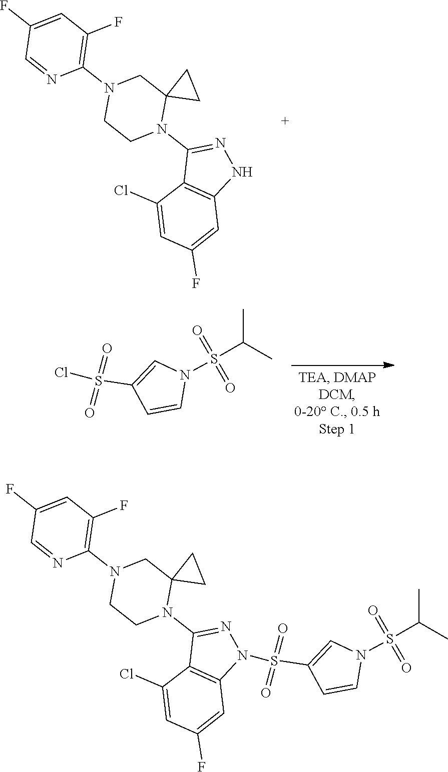 [SMILES: Fc1cnc(N2CCN(c3n[nH]c4cc(F)cc(Cl)c34)C3(CC3)C2)c(F)c1 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cc4F)CC34CC4)c3c(Cl)cc(F)cc32)c1]

Step 1: To a solution of 1-isopropylsulfonylpyrrole-3-sulfonyl chloride (138 mg, 508 mol, 2 eq) and 4-chloro-3-[7-(3, 5-difluoro-2-pyridyl)-4, 7-diazaspiro [2.5]octan-4-yl]-6-fluoro-1H-indazole (100 mg, 254 μmol, 1 eq) in DCM (1 mL) was added TEA (51.4 mg, 508 μmol, 70.7 μL, 2 eq) and DMAP (3.10 mg, 25.4 μmol, 0.1 eq) at 0° C. The mixture was stirred at 20° C. for 0.5 hour. LC-MS showed 4-chloro-3-[7-(3, 5-difluoro-2-pyridyl)-4, 7-diazaspiro [2.5]octan-4-yl]-6-fluoro-1H-indazole remained and a little desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (basic condition; Method: column: Phenomenex C18 75*30 mm*3 um; mobile phase: [water (NH₃H₂O+NH₄HCO₃)-ACN]; B %: 50%-98%, 8 min) to give desired 4-chloro-3-[7-(3, 5-difluoro-2-pyridyl)-4, 7-diazaspiro [2.5]octan-4-yl]-6-fluoro-1-(1-isopropylsulfonylpyrrol-3-yl) sulfonyl-indazole (26.3 mg, 38.4 μmol, 40.2% yield, 91.77% purity) as a white solid. ¹H NMR (400 MHz, DMSO-d₆) δ 8.13-8.09 (m, 1H), 8.06-8.01 (m, 1H), 7.86-7.76 (m, 2H), 7.58-7.48 (m, 1H), 7.37-7.31 (m, 1H), 6.57 (dd, J=1.6, 3.3 Hz, 1H), 3.98-3.88 (m, 1H), 3.69-3.46 (m, 4H), 3.39-3.32 (m, 2H), 1.12-1.04 (m, 6H), 0.82-0.72 (m, 2H), 0.49 (br s, 2H) HPLC: 91.77% (220 nm), 98.55% (215 nm), 96.27% (254 nm). MS (ESI): mass calcd. For C₂₅H₂₄ClF₃N₆O₄S₂ 628.09 m/z found 629.1 [M+H]⁺.

### Compound 159: 3-(7-(3, 5-difluoropyridin-2-yl)-4, 7-diazaspiro [2.5]octan-4-yl)-4, 6-difluoro-1-((1-(isopropylsulfonyl)-1H-pyrrol-3-yl) sulfonyl)-1H-indazole

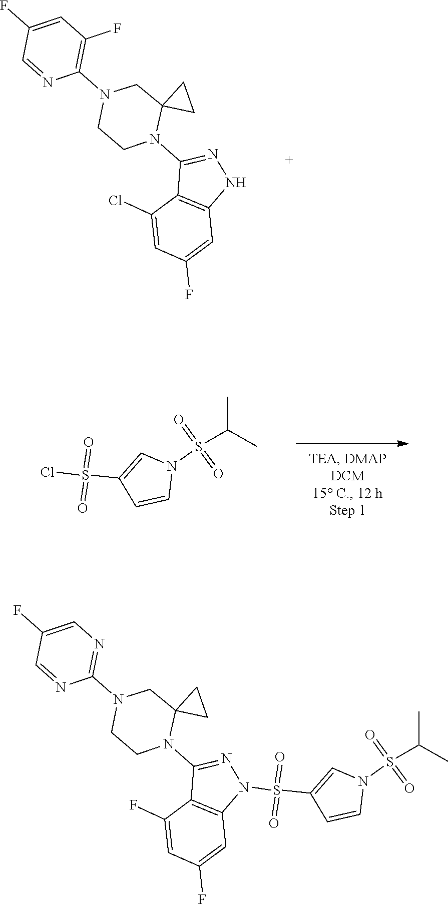 [SMILES: Fc1cnc(N2CCN(c3n[nH]c4cc(F)cc(Cl)c34)C3(CC3)C2)c(F)c1 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cn4)CC34CC4)c3c(F)cc(F)cc32)c1]

Step 1: To a solution of 3-[7-(3, 5-difluoro-2-pyridyl)-4, 7-diazaspiro [2.5]octan-4-yl]-4, 6-difluoro-1H-indazole (30 mg, 79.5 μmol, 1 eq) and 1-isopropylsulfonylpyrrole-3-sulfonyl chloride (32.4 mg, 119 μmol, 1.5 eq) in DCM (1 mL) was added TEA (16.1 mg, 159 μmol, 22.1 μL, 2 eq) and DMAP (971 μg, 7.95 μmol, 0.1 eq). The mixture was stirred at 15° C. for 12 hours. LC-MS showed 3-[7-(3, 5-difluoro-2-pyridyl)-4, 7-diazaspiro [2.5]octan-4-yl]-4, 6-difluoro-1H-indazole was consumed completely and desired mass was detected. The reaction mixture was concentrated under reduced pressure to give a residue. The residue was purified by prep-TLC (petroleum ether/Ethyl acetate=3/1) to give desired 3-(7-(3, 5-difluoropyridin-2-yl)-4, 7-diazaspiro [2.5]octan-4-yl)-4, 6-difluoro-1-((1-(isopropylsulfonyl)-1H-pyrrol-3-yl) sulfonyl)-1H-indazole (6.7 mg, 10.9 μmol, 13.8% yield, 100.00% purity) as a white solid. ¹H NMR (400 MHz, DMSO-d₆) δ 8.06 (d, J=2.2 Hz, 1H), 7.97 (s, 1H), 7.82-7.73 (m, 1H), 7.66-7.60 (m, 1H), 7.34-7.28 (m, 2H), 6.53-6.49 (m, 1H), 3.94-3.85 (m, 1H), 3.72-3.65 (m, 2H), 3.55-3.37 (m, 2H), 3.28-3.14 (m, 2H), 1.03 (d, J=6.8 Hz, 6H), 0.85-0.79 (m, 2H), 0.57 (br s, 2H). HPLC: 100.00% (220 nm), 99.61% (215 nm), 100.00% (254 nm). MS (ESI): mass calcd. For C₂₅H₂₄F₄N₆O₄S₂ 612.12 m/z found 613.2 [M+H]⁺.

### Compound 160: 4-chloro-3-[7-(3, 5-difluoro-2-pyridyl)-4, 7-diazaspiro [2.5]octan-4-yl]-6-fluoro-1-(1-isopropylsulfonyl-2, 5-dimethyl-pyrrol-3-yl) sulfonyl-indazole

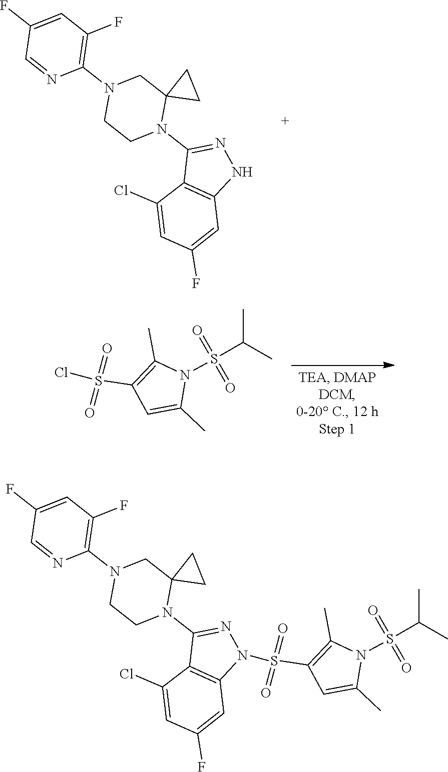 [SMILES: Fc1cnc(N2CCN(c3n[nH]c4cc(F)cc(Cl)c34)C3(CC3)C2)c(F)c1 ; Cc1cc(S(=O)(=O)Cl)c(C)n1S(=O)(=O)C(C)C ; Cc1cc(S(=O)(=O)n2nc(N3CCN(c4ncc(F)cc4F)CC34CC4)c3c(Cl)cc(F)cc32)c(C)n1S(=O)(=O)C(C)C]

Step 1: To a solution of 4-chloro-3-[7-(3, 5-difluoro-2-pyridyl)-4, 7-diazaspiro [2.5]octan-4-yl]-6-fluoro-1H-indazole (17.1 mg, 43.4 μmol, 1 eq) and 1-isopropylsulfonyl-2, 5-dimethyl-pyrrole-3-sulfonyl chloride (13 mg, 43.4 μmol, 1 eq) in DCM (1 mL) was added TEA (4.39 mg, 43.4 μmol, 6.04 μL, 1 eq) and DMAP (5.30 mg, 43.4 μmol, 1 eq) at 0° C. The mixture was stirred at 25° C. for 12 hours. LC-MS showed 4-chloro-3-[7-(3, 5-difluoro-2-pyridyl)-4, 7-diazaspiro [2.5]octan-4-yl]-6-fluoro-1H-indazole was consumed completely and desired mass was detected. The reaction mixture was added to water (10 mL), the aqueous phase was extracted with DCM (20 mL*3). The organic layer was dried over Na₂SO₄, filtered and the filtrate was concentrated to give a residue. The residue was purified by prep-TLC (SiO₂, petroleum ether/Ethyl acetate=3/1) to give desired 4-chloro-3-[7-(3, 5-difluoro-2-pyridyl)-4, 7-diazaspiro [2.5]octan-4-yl]-6-fluoro-1-(1-isopropylsulfonyl-2, 5-dimethyl-pyrrol-3-yl) sulfonyl-indazole (15 mg, 22.6 μmol, 52.1% yield, 99.00% purity) as a white solid. ¹H NMR (400 MHz, DMSO-d₆) δ 8.17 (d, J=2.5 Hz, 1H), 7.92-7.79 (m, 2H), 7.58 (dd, J=2.0, 9.1 Hz, 1H), 6.45 (s, 1H), 3.89-3.78 (m, 1H), 3.68 (br t, J=4.6 Hz, 2H), 3.58 (br s, 2H), 2.72 (s, 3H), 2.28 (s, 3H), 2.14 (s, 2H), 1.21 (s, 3H), 1.19 (s, 3H), 0.84 (br s, 2H), 0.58 (br s, 2H). HPLC: 99.00% (220 nm), 99.39% (215 nm), 99.07% (254 nm). MS (ESI): mass calcd. For C₂₇H₂₈ClF₃N₆O₄S₂ 656.13, m/z found 657.1 [M+H]⁺.

### Compound 161: 4-chloro-6-fluoro-3-[7-(5-fluoro-2-pyridyl)-4, 7-diazaspiro [2.5]octan-4-yl]-1-(1-isopropylsulfonylpyrrol-3-yl) sulfonyl-indazole

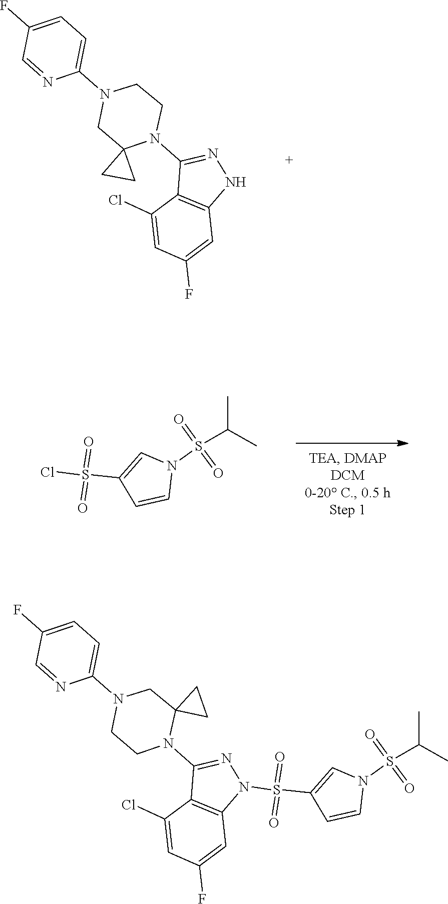 [SMILES: Fc1ccc(N2CCN(c3n[nH]c4cc(F)cc(Cl)c34)C3(CC3)C2)nc1 ; CC(C)S(=O)(=O)n1ccc(S(=O)(=O)Cl)c1 ; CC(C)S(=O)(=O)n1ccc(S(=O)(=O)n2nc(N3CCN(c4ccc(F)cn4)CC34CC4)c3c(Cl)cc(F)cc32)c1]

Step 1: To a solution of 4-chloro-6-fluoro-3-[7-(5-fluoro-2-pyridyl)-4, 7-diazaspiro [2.5]octan-4-yl]-1H-indazole (50.0 mg, 133 μmol, 1 eq) and 1-isopropylsulfonylpyrrole-3-sulfonyl chloride (54.2 mg, 200 μmol, 1.5 eq) in DCM (2 mL) was added TEA (26.9 mg, 266 μmol, 37.0 μL, 2 eq) and DMAP (1.63 mg, 13.3 μmol, 0.1 eq) at 0° C. The mixture was stirred at 20° C. for 0.5 hour. LC-MS showed 4-chloro-6-fluoro-3-[7-(5-fluoro-2-pyridyl)-4, 7-diazaspiro [2.5]octan-4-yl]-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Waters Xbridge Prep OBD C18 150*40 mm*10 um; mobile phase: [water(NH₄HCO₃)-ACN]; B %: 55%-90%, 8 min) to give desired 4-chloro-6-fluoro-3-[7-(5-fluoro-2-pyridyl)-4, 7-diazaspiro [2.5]octan-4-yl]-1-(1-isopropylsulfonylpyrrol-3-yl) sulfonyl-indazole (4.4 mg, 7.20 μmol, 5.41% yield, 100.00% purity) as a white solid. ¹H NMR (400 MHz, DMSO-d₆) δ 8.15 (d, J=3.1 Hz, 1H), 8.10 (t, J=1.9 Hz, 1H), 7.86 (dd, J=2.0, 8.9 Hz, 1H), 7.63-7.54 (m, 2H), 7.39 (dd, J=2.5, 3.1 Hz, 1H), 6.95 (dd, J=3.3, 9.3 Hz, 1H), 6.62 (dd, J=1.6, 3.3 Hz, 1H), 4.05-3.91 (m, 1H), 3.71 (s, 2H), 3.63 (br t, J=4.6 Hz, 2H), 3.51 (br s, 2H), 1.12 (d, J=6.8 Hz, 6H), 0.85 (s, 2H), 0.54 (s, 2H). HPLC: 100.00% (220 nm), 99.91% (215 nm), 100.00% (254 nm). MS (ESI): mass calcd. For C₂₅H₂₅ClF₂N₆O₄S₂ 610.10, m/z found 611.1 [M+H]⁺.

### Compound 162: 4-chloro-6-fluoro-3-[7-(3-fluoro-2-pyridyl)-4, 7-diazaspiro [2.5]octan-4-yl]-1-(1-isopropylsulfonylpyrrol-3-yl) sulfonyl-indazole

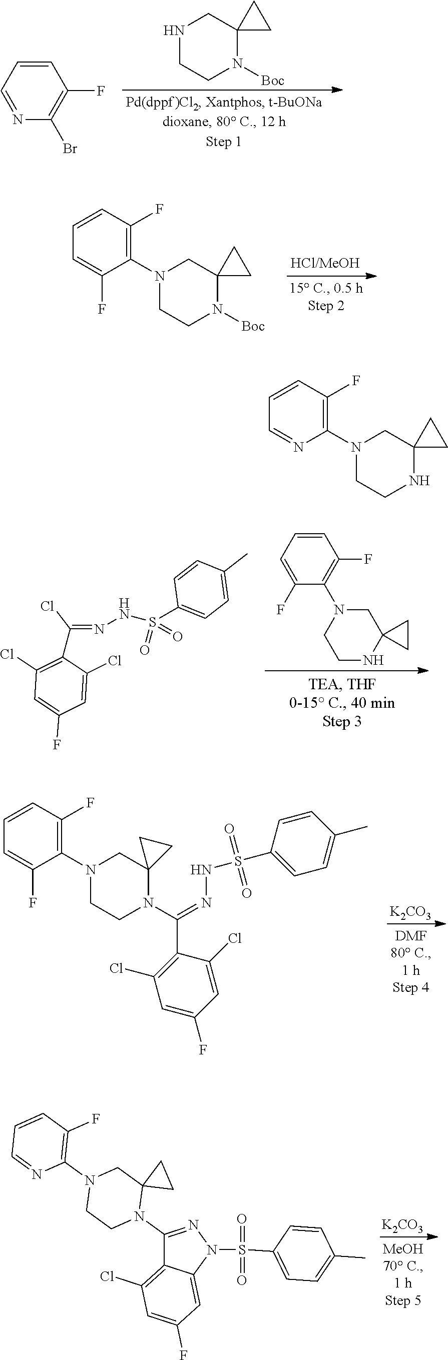

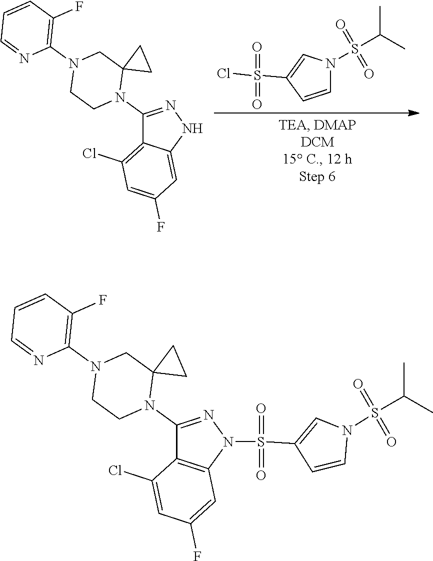 [SMILES: Fc1cc(Cl)c2c(N3CCN(c4ncccc4F)CC34CC4)n[nH]c2c1 ; CC(C)S(=O)(=O)n1ccc(S(=O)(=O)Cl)c1 ; CC(C)S(=O)(=O)n1ccc(S(=O)(=O)n2nc(N3CCN(c4ncccc4F)CC34CC4)c3c(Cl)cc(F)cc32)c1]

Step 1: A mixture of 2-bromo-3-fluoro-pyridine (300 mg, 1.70 mmol, 1 eq), tert-butyl 4, 7-diazaspiro [2.5]octane-4-carboxylate (362 mg, 1.70 mmol, 1 eq), Xantphos (197 mg, 345 μmol, 0.2 eq), t-BuONa (492 mg, 5.11 mmol, 3 eq) and Pd(dppf)Cl₂ (125 mg, 171 μmol, 0.1 eq) in dioxane (10 mL) was degassed and purged with N₂ for 3 times, and then the mixture was stirred at 80° C. for 12 hours under N₂ atmosphere. LC-MS showed 2-bromo-3-fluoro-pyridine was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by flash silica gel chromatography (ISCO®; 12 SepaFlash® Silica Flash Column, Eluent of 0˜15% EtOAc/petroleum ether gradient @60 mL/min) to give desired tert-butyl 7-(3-fluoro-2-pyridyl)-4, 7-diazaspiro [2.5]octane-4-carboxylate (260 mg, 846 μmol, 49.6% yield) as a white solid. MS (ESI): mass calcd. For C₁₆H₂₂FN₃O₂ 307.17 found 308.1 [M+H]⁺.

Step 2: To a solution of tert-butyl 7-(3-fluoro-2-pyridyl)-4, 7-diazaspiro [2.5]octane-4-carboxylate (260 mg, 846 μmol, 1 eq) in HCl/MeOH (5 mL). The mixture was stirred at 15° C. for 0.5 hour. TLC (petroleum ether/EtOAc=5/1) indicated 7-(3-fluoro-2-pyridyl)-4, 7-diazaspiro [2.5]octane-4-carboxylate was consumed completely and one new spot formed. The reaction mixture was filtered and the filtrate was concentrated under reduced pressure to give desired 7-(3-fluoro-2-pyridyl)-4, 7-diazaspiro [2.5]octane (0.26 g, crude) as a white solid.

Step 3: To a solution of 7-(3-fluoro-2-pyridyl)-4, 7-diazaspiro [2.5]octane (0.26 g, 1.07 mmol, 1 eq, HCl) in THF (4 mL) was added dropwise TEA (1.08 g, 10.7 mmol, 1.48 mL, 10 eq) at 25° C. After addition, the mixture was stirred at this temperature for 10 mins, and then (1Z)-2, 6-dichloro-4-fluoro-N-(p-tolylsulfonyl) benzohydrazonoyl chloride (464 mg, 1.17 mmol, 1.1 eq) in THF (2 mL) was added dropwise at 0° C. The resulting mixture was stirred at 25° C. for 0.5 hour. LC-MS showed 7-(3-fluoro-2-pyridyl)-4, 7-diazaspiro [2.5]octane was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N—[(Z)-[(2, 6-dichloro-4-fluoro-phenyl)-[7-(3-fluoro-2-pyridyl)-4, 7-diazaspiro [2.5]octan-4-yl]methylene]amino]-4-methyl-benzenesulfonamide (960 mg, crude) as a white solid. MS (ESI): mass calcd. For C₂₅H₂₃Cl₂F₂N₅O₂S 565.1 found 566.1 [M+H]⁺.

Step 4: To a solution of N—[(Z)-[(2, 6-dichloro-4-fluoro-phenyl)-[7-(3-fluoro-2-pyridyl)-4, 7-diazaspiro [2.5]octan-4-yl]methylene]amino]-4-methyl-benzenesulfonamide (480 mg, 847 μmol, 1 eq) in DMF (10 mL) was added K₂CO₃ (586 mg, 4.24 mmol, 5 eq). The mixture was stirred at 80° C. for 1 hour. LC-MS showed N—[(Z)-[(2, 6-dichloro-4-fluoro-phenyl)-[7-(3-fluoro-2-pyridyl)-4, 7-diazaspiro [2.5]octan-4-yl]methylene]amino]-4-methyl-benzenesulfonamide was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired 4-chloro-6-fluoro-3-[7-(3-fluoro-2-pyridyl)-4, 7-diazaspiro [2.5]octan-4-yl]-1-(p-tolylsulfonyl) indazole (670 mg, crude) as a yellow solid. MS (ESI): mass calcd. For C₂₅H₂₂ClF₂N₅O₂S 529.1 found 530.1 [M+H]⁺.

Step 5: To a solution of 4-chloro-6-fluoro-3-[7-(3-fluoro-2-pyridyl)-4, 7-diazaspiro [2.5]octan-4-yl]-1-(p-tolylsulfonyl) indazole (670 mg, 1.26 mmol, 1 eq) in MeOH (5 mL) was added K₂CO₃ (874 mg, 6.32 mmol, 5 eq). The mixture was stirred at 70° C. for 1 hour. LC-MS showed 4-chloro-6-fluoro-3-[7-(3-fluoro-2-pyridyl)-4, 7-diazaspiro [2.5]octan-4-yl]-1-(p-tolylsulfonyl) indazole was consumed completely and desired mass was detected. The reaction mixture was filtered and the filtrate was concentrated under reduced pressure to give a residue. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether/EtOAc=2/1) to give desired 4-chloro-6-fluoro-3-[7-(3-fluoro-2-pyridyl)-4, 7-diazaspiro [2.5]octan-4-yl]-1H-indazole (240 mg, 639 μmol, 50.52% yield) as a yellow solid. MS (ESI): mass calcd. For C₁₈H₁₆ClF₂N₅ 375.1 found 376.1 [M+H]⁺.

Step 6: To a solution of 4-chloro-6-fluoro-3-[7-(3-fluoro-2-pyridyl)-4, 7-diazaspiro [2.5]octan-4-yl]-1H-indazole (50 mg, 133 μmol, 1 eq) and 1-isopropylsulfonylpyrrole-3-sulfonyl chloride (54.2 mg, 200 μmol, 1.5 eq) in DCM (2 mL) was added TEA (26.9 mg, 266 μmol, 37.0 μL, 2 eq) and DMAP (1.63 mg, 13.3 μmol, 0.1 eq). The mixture was stirred at 15° C. for 12 hours. LC-MS showed 4-chloro-6-fluoro-3-[7-(3-fluoro-2-pyridyl)-4, 7-diazaspiro [2.5]octan-4-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-HPLC (FA condition; column: Phenomenex Luna C18 75*30 mm*3 um; mobile phase: [water (FA)-ACN]; B %: 30%-90%, 8 min) to give desired 4-chloro-6-fluoro-3-[7-(3-fluoro-2-pyridyl)-4, 7-diazaspiro [2.5]octan-4-yl]-1-(1-isopropylsulfonylpyrrol-3-yl)sulfonyl-indazole (40.8 mg, 66.0 μmol, 49.6% yield, 98.85% purity) as a white solid. ¹H NMR (400 MHz, DMSO-d₆) δ 8.07-8.04 (m, 1H), 8.03-7.99 (m, 1H), 7.80 (dd, J=1.8, 8.9 Hz, 1H), 7.56-7.48 (m, 2H), 7.35 (t, J=2.8 Hz, 1H), 6.92-6.87 (m, 1H), 6.60-6.55 (m, 1H), 3.93 (td, J=6.8, 13.5 Hz, 1H), 3.61 (br s, 4H), 3.48-3.37 (m, 2H), 1.06 (d, J=6.7 Hz, 6H), 0.78 (br s, 2H), 0.53-0.43 (m, 2H). HPLC: 98.85% (220 nm), 99.09% (215 nm), 98.87% (254 nm). MS (ESI): mass calcd. For C₂₅H₂₅ClF₂N₆O₄S₂ 610.10 m/z found 611.1 [M+H]⁺.

### Compound 163: 4-chloro-6-fluoro-1-(1-isopropylsulfonylpyrrol-3-yl)sulfonyl-3-[7-(2-pyridyl)-4,7-diazaspiro[2.5]octan-4-yl]indazole

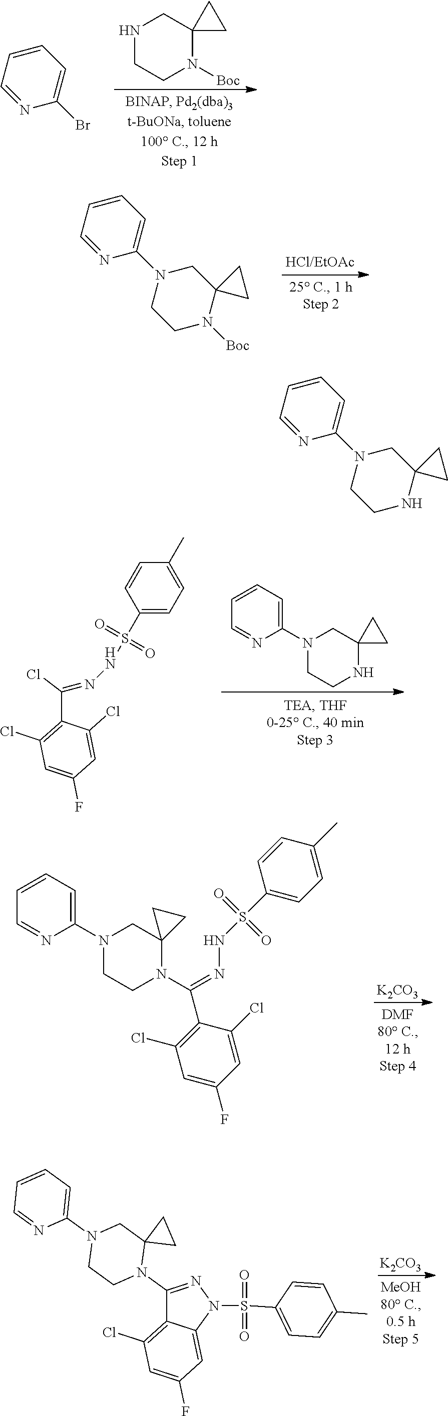

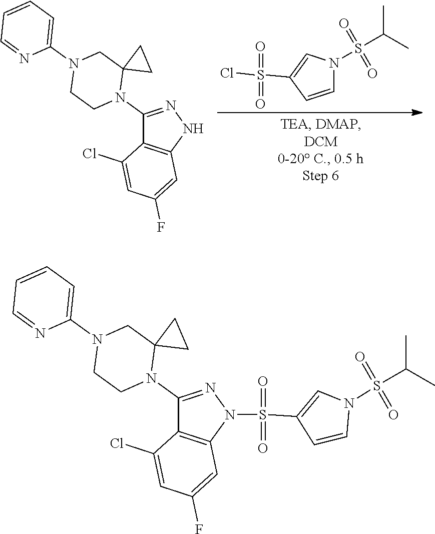 [SMILES: Fc1cc(Cl)c2c(N3CCN(c4ccccn4)CC34CC4)n[nH]c2c1 ; CC(C)S(=O)(=O)n1ccc(S(=O)(=O)Cl)c1 ; CC(C)S(=O)(=O)n1ccc(S(=O)(=O)n2nc(N3CCN(c4ccccn4)CC34CC4)c3c(Cl)cc(F)cc32)c1]

Step 1: A mixture of tert-butyl 4,7-diazaspiro[2.5]octane-4-carboxylate (806 mg, 3.80 mmol, 2 eq), 2-bromopyridine (300 mg, 1.90 mmol, 181 μL, 1 eq), BINAP (118.23 mg, 190 μmol, 0.1 eq), Pd₂(dba)₃ (86.9 mg, 94.9 μmol, 0.05 eq) and t-BuONa (547 mg, 5.70 mmol, 3 eq) in Tol (10 mL) was degassed and purged with N₂ for 3 times, and then the mixture was stirred at 100° C. for 12 hours under N₂ atmosphere. TLC indicated tert-butyl 4, 7-diazaspiro [2.5]octane-4-carboxylate was consumed completely and one new spot formed. The reaction was clean according to TLC. The reaction mixture was added to water (20 mL), the aqueous phase was extracted with EtOAc (20 mL*3). The organic layer was dried over Na₂SO₄, filtered and the filtrate was concentrated to give a residue. The residue was purified by column chromatography (SiO₂, petroleum ether/EtOAc=1/0 to 0/1) to give desired tert-butyl 7-(2-pyridyl)-4, 7-diazaspiro [2.5]octane-4-carboxylate (440 mg, crude) as a white solid.

Step 2: To a solution of tert-butyl 7-(2-pyridyl)-4, 7-diazaspiro [2.5]octane-4-carboxylate (440 mg, 1.52 mmol, 1 eq) in HCl/EtOAc (5 mL, 4M) was stirred at 25° C. for 1 hour. LC-MS showed desired mass was detected. The reaction mixture was filtered and the filter liquor was concentrated under reduced pressure to give desired 7-(2-pyridyl)-4, 7-diazaspiro [2.5]octane (500 mg, crude, HCl) as a white solid. MS (ESI): mass calcd. For C₁₁H₁₅N₃ 189.13, m/z found 190.1 [M+H]⁺.

Step 3: To a solution of (1Z)-2, 6-dichloro-4-fluoro-N-(p-tolylsulfonyl) benzohydrazonoyl chloride (418 mg, 1.06 mmol, 1 eq) in THF (3 mL) was added dropwise TEA (1.07 g, 10.6 mmol, 1.47 mL, 10 eq) at 25° C. After addition, the mixture was stirred at this temperature for 10 mins, and then 7-(2-pyridyl)-4, 7-diazaspiro [2.5]octane (200 mg, 1.06 mmol, 1 eq) in THF (1 mL) was added dropwise at 0° C. The resulting mixture was stirred at 25° C. for 0.5 hour. LC-MS showed desired mass was detected. The reaction mixture was added to water (20 mL), the aqueous phase was extracted with EtOAc (20 mL*3). The organic layer was dried over Na₂SO₄, filtered and the filtrate was concentrated to give desired N—[(Z)-[(2,6-dichloro-4-fluoro-phenyl)-[7-(2-pyridyl)-4, 7-diazaspiro [2.5]octan-4-yl]methylene]amino]-4-methyl-benzenesulfonamide (460 mg, crude) as a white solid. MS (ESI): mass calcd. For C₂₅H₂₄Cl₂FN₅O₂S 547.1, m/z found 548.1 [M+H]⁺.

Step 4: To a solution of N—[(Z)-[(2, 6-dichloro-4-fluoro-phenyl)-[7-(2-pyridyl)-4, 7-diazaspiro [2.5]octan-4-yl]methylene]amino]-4-methyl-benzenesulfonamide (460 mg, 839 μmol, 1 eq) in DMF (5 mL) was added K₂CO₃ (579.58 mg, 4.19 mmol, 5 eq). The mixture was stirred at 80° C. for 12 hours. LC-MS showed desired mass was detected. The reaction mixture was added to water (20 mL), the aqueous phase was extracted with EtOAc (20 mL*3). The organic layer was dried over Na₂SO₄, filtered and the filtrate was concentrated to give desired 4-chloro-6-fluoro-1-(p-tolylsulfonyl)-3-[7-(2-pyridyl)-4, 7-diazaspiro [2.5]octan-4-yl]indazole (450 mg, crude) as a white solid. MS (ESI): mass calcd. For C₂₅H₂₃ClFN₅O₂S 511.12, m/z found 512.1 [M+H]⁺.

Step 5: To a solution of 4-chloro-6-fluoro-1-(p-tolylsulfonyl)-3-[7-(2-pyridyl)-4, 7-diazaspiro [2.5]octan-4-yl]indazole (450 mg, 879 μmol, 1 eq) in MeOH (5 mL) was added K₂CO₃ (486 mg, 3.52 mmol, 4 eq). The mixture was stirred at 80° C. for 0.5 hour. LC-MS showed 4-chloro-6-fluoro-1-(p-tolylsulfonyl)-3-[7-(2-pyridyl)-4, 7-diazaspiro [2.5]octan-4-yl]indazole was consumed completely and desired mass was detected. The reaction mixture was added to water (20 mL), the aqueous phase was extracted with EtOAc (20 mL*3). The organic layer was dried over Na₂SO₄, filtered and the filtrate was concentrated to give a residue. The residue was purified by prep-TLC (SiO₂, petroleum ether/EtOAc=1/1) to give desired 4-chloro-6-fluoro-3-[7-(2-pyridyl)-4, 7-diazaspiro [2.5]octan-4-yl]-1H-indazole (140 mg, crude) as a white solid. MS (ESI): mass calcd. For C₁₈H₁₇ClFN₅ 357.12, m/z found 358.1 [M+H]⁺.

Step 6: To a solution of 4-chloro-6-fluoro-3-[7-(2-pyridyl)-4, 7-diazaspiro [2.5]octan-4-yl]-1H-indazole (40 mg, 112 μmol, 1 eq) and 1-isopropylsulfonylpyrrole-3-sulfonyl chloride (60.8 mg, 224 μmol, 2 eq) in DCM (2 mL) was added TEA (22.6 mg, 224 μmol, 31.1 μL, 2 eq) and DMAP (1.37 mg, 11.2 μmol, 0.1 eq) at 0° C. The mixture was stirred at 20° C. for 0.5 hour. LC-MS showed 4-chloro-6-fluoro-3-[7-(2-pyridyl)-4, 7-diazaspiro [2.5]octan-4-yl]-1H-indazole was consumed completely and desired mass was detected. The reaction mixture was filtered and the filter liquor was concentrated under reduced pressure to give a residue. The residue was purified by prep-HPLC (FA condition; column: Phenomenex Luna C18 75*30 mm*3 um; mobile phase: [water(FA)-ACN];B %: 30%-70%, 8 mins) to give desired 4-chloro-6-fluoro-1-(1-isopropylsulfonylpyrrol-3-yl)sulfonyl-3-[7-(2-pyridyl)-4,7-diazaspiro[2.5]octan-4-yl]indazole (16.4 mg, 25.20 μmol, 22.54% yield, 91.14% purity) as a white solid. ¹H NMR (400 MHz, DMSO-d₆) δ 8.11 (br d, J=4.3 Hz, 1H), 8.05 (s, 1H), 7.81 (br d, J=9.3 Hz, 1H), 7.58-7.52 (m, 2H), 7.34 (t, J=2.7 Hz, 1H), 6.85 (d, J=8.9 Hz, 1H), 6.69-6.63 (m, 1H), 6.58-6.56 (m, 1H), 3.97-3.90 (m, 1H), 3.71 (s, 2H), 3.60-3.50 (m, 4H), 1.07 (d, J=6.8 Hz, 6H), 0.81 (s, 2H), 0.49 (s, 2H). HPLC: 91.14% (220 nm), 90.90% (210 nm), 91.59% (254 nm). MS (ESI): mass calcd. For C₂₅H₂₆ClFN₆O₄S₂ 592.11, m/z found 593.1 [M+H]⁺.

### Compound 164: 4-chloro-6-fluoro-1-(1-isopropylsulfonylpyrrol-3-yl)sulfonyl-3-(7-pyrimidin-2-yl-4,7-diazaspiro[2.5]octan-4-yl) indazole

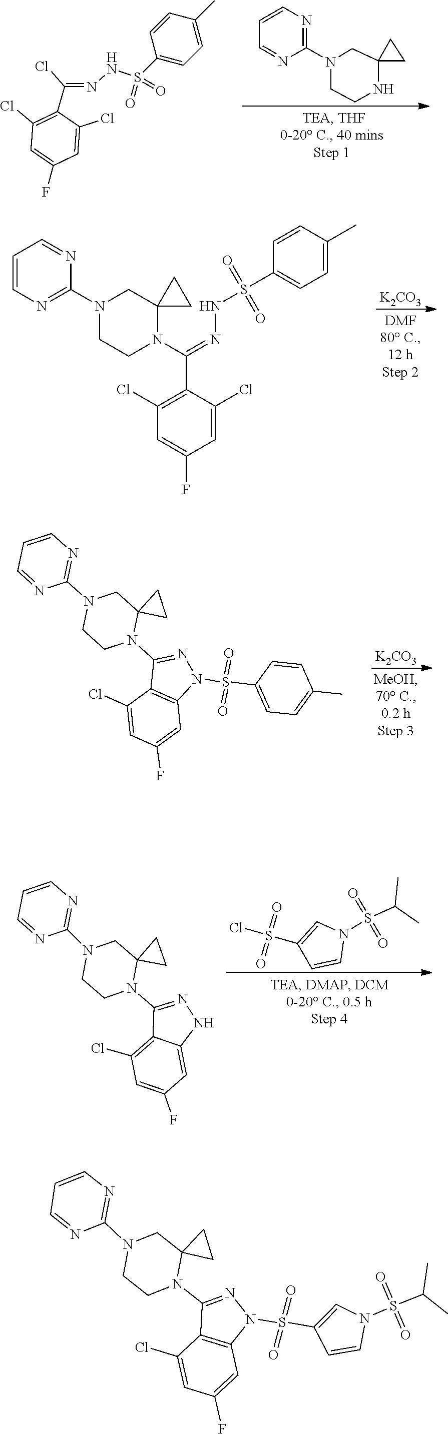

Step 1: To a solution of (1Z)-2, 6-dichloro-4-fluoro-N-(p-tolylsulfonyl)benzohydrazonoyl chloride (416 mg, 1.05 mmol, 1 eq) in THF (3 mL) was added dropwise TEA (1.06 g, 10.5 mmol, 1.46 mL, 10 eq) at 25° C. After addition, the mixture was stirred at this temperature for 10 mins, and then 7-pyrimidin-2-yl-4, 7-diazaspiro [2.5]octane (200 mg, 1.05 mmol, 1 eq) in THF (1 mL) was added dropwise at 0° C. The mixture was stirred at 25° C. for 0.5 hour. LC-MS showed (1Z)-2, 6-dichloro-4-fluoro-N-(p-tolylsulfonyl)benzohydrazonoyl chloride was consumed completely and desired mass was detected. The reaction mixture was added to water (20 mL), the aqueous phase was extracted with EtOAc (20 mL*3). The organic layer was dried over Na₂SO₄, filtered and the filtrate was concentrated to give desired N—[(Z)-[(2, 6-dichloro-4-fluoro-phenyl)-(7-pyrimidin-2-yl-4, 7-diazaspiro [2.5]octan-4-yl) methylene]amino]-4-methyl-benzenesulfonamide (480 mg, crude) as a white solid. MS (ESI): mass calcd. For C₂₄H₂₃Cl₂FN₆O₂S 548.1, m/z found 549.1 [M+H]⁺.

Step 2: To a solution of N—[(Z)-[(2, 6-dichloro-4-fluoro-phenyl)-(7-pyrimidin-2-yl-4, 7-diazaspiro [2.5]octan-4-yl) methylene]amino]-4-methyl-benzenesulfonamide (480 mg, 874 μmol, 1 eq) in DMF (5 mL) was added K₂CO₃ (603.71 mg, 4.37 mmol, 5 eq). The mixture was stirred at 80° C. for 12 hours. LC-MS showed desired mass was detected. The reaction mixture was added to water (20 mL), the aqueous phase was extracted with EtOAc (20 mL*3). The organic layer was dried over Na₂SO₄, filtered and the filtrate was concentrated to give desired 4-chloro-6-fluoro-1-(p-tolylsulfonyl)-3-(7-pyrimidin-2-yl-4, 7-diazaspiro [2.5]octan-4-yl) indazole (450 mg, crude) as a white solid. MS (ESI): mass calcd. For C₂₄H₂₂ClFN₆O₂S 512.12, m/z found 513.1 [M+H]⁺.

Step 3: To a solution of 4-chloro-6-fluoro-1-(p-tolylsulfonyl)-3-(7-pyrimidin-2-yl-4, 7-diazaspiro [2.5]octan-4-yl) indazole (450 mg, 877 μmol, 1 eq) in MeOH (5 mL) was added K₂CO₃ (484.95 mg, 3.51 mmol, 4 eq). The mixture was stirred at 80° C. for 0.5 hour. LC-MS showed 4-chloro-6-fluoro-1-(p-tolylsulfonyl)-3-(7-pyrimidin-2-yl-4, 7-diazaspiro [2.5]octan-4-yl) indazole was consumed completely and desired mass was detected. The reaction mixture was added to water (20 mL), the aqueous phase was extracted with EtOAc (20 mL*3). The organic layer was dried over Na₂SO₄, filtered and the filtrate was concentrated to give a residue. The residue was purified by prep-TLC (SiO₂, petroleum ether/EtOAc=1/1) to give desired 4-chloro-6-fluoro-3-(7-pyrimidin-2-yl-4, 7-diazaspiro [2.5]octan-4-yl)-1H-indazole (160 mg, crude) as a white solid. MS (ESI): mass calcd. For C₁₇H₁₆ClFN₆ 358.11, m/z found 359.1 [M+H]⁺.

Step 4: To a solution of 4-chloro-6-fluoro-3-(7-pyrimidin-2-yl-4, 7-diazaspiro [2.5]octan-4-yl)-1H-indazole (50 mg, 139 μmol, 1 eq) and 1-isopropylsulfonylpyrrole-3-sulfonyl chloride (56.8 mg, 209 μmol, 1.5 eq) in DCM (2 mL) was added TEA (28.2 mg, 279 μmol, 38.8 μL, 2 eq) and DMAP (1.70 mg, 13.9 μmol, 0.1 eq) at 0° C. The mixture was stirred at 20° C. for 0.5 hour. LC-MS showed 4-chloro-6-fluoro-3-(7-pyrimidin-2-yl-4, 7-diazaspiro [2.5]octan-4-yl)-1H-indazole was consumed completely and desired mass was detected. The reaction mixture was filtered and the filter liquor was concentrated under reduced pressure to give a residue. The residue was purified by prep-TLC (SiO₂, petroleum ether/EtOAc=3/1) to give desired 4-chloro-6-fluoro-1-(1-isopropylsulfonylpyrrol-3-yl) sulfonyl-3-(7-pyrimidin-2-yl-4, 7-diazaspiro [2.5]octan-4-yl) indazole (15 mg, 25.0 μmol, 17.9% yield, 98.88% purity) as a white solid. ¹H NMR (400 MHz, DMSO-d₆) δ 8.40-8.34 (m, 2H), 8.06 (s, 1H), 7.83-7.78 (m, 1H), 7.54 (dd, J=2.0, 9.3 Hz, 1H), 7.38-7.33 (m, 1H), 6.67-6.63 (m, 1H), 6.60-6.57 (m, 1H), 3.99-3.91 (m, 3H), 3.79-3.72 (m, 1H), 3.55-3.51 (m, 2H), 1.24-1.20 (m, 1H), 1.06 (d, J=6.8 Hz, 6H), 0.81-0.76 (m, 2H), 0.50-0.44 (m, 2H). HPLC: 98.88% (220 nm), 99.08% (215 nm), 98.80% (254 nm). MS (ESI): mass calcd. For C₂₄H₂₅ClFN₇O₄S₂ 593.11, m/z found 594.1 [M+H]⁺.

### Compound 165: 4, 6-difluoro-3-(7-(5-fluoropyridin-2-yl)-4, 7-diazaspiro [2.5]octan-4-yl)-1-((1-(isopropylsulfonyl)-1H-pyrrol-3-yl) sulfonyl)-1H-indazole

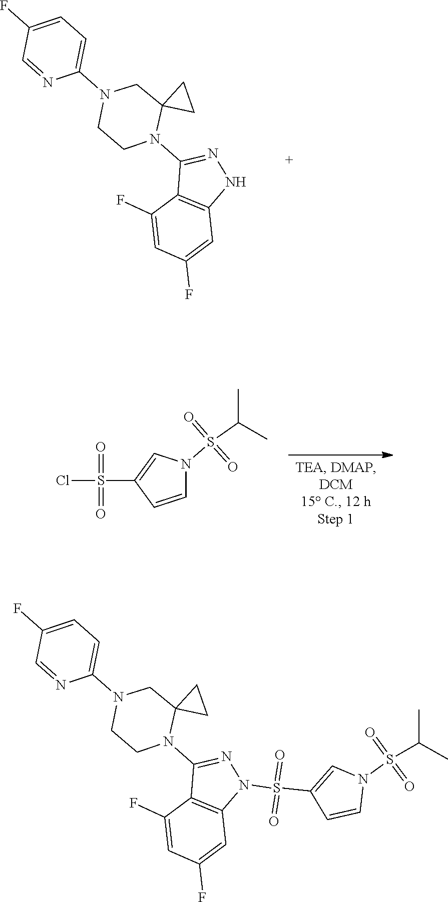 [SMILES: Fc1ccc(N2CCN(c3n[nH]c4cc(F)cc(F)c34)C3(CC3)C2)nc1 ; CC(C)S(=O)(=O)n1ccc(S(=O)(=O)Cl)c1 ; CC(C)S(=O)(=O)n1ccc(S(=O)(=O)n2nc(N3CCN(c4ccc(F)cn4)CC34CC4)c3c(F)cc(F)cc32)c1]

Step 1: To a solution of 4,6-difluoro-3-[7-(5-fluoro-2-pyridyl)-4, 7-diazaspiro[2.5]octan-4-yl]-1H-indazole (60 mg, 167 μmol, 1 eq) and 1-isopropylsulfonylpyrrole-3-sulfonyl chloride (68.1 mg, 250 umol, 1.5 eq) in DCM (1 mL) was added TEA (33.8 mg, 334 umol, 46.5 μL, 2 eq) and DMAP (2.04 mg, 16.7 μmol, 0.1 eq). The mixture was stirred at 15° C. for 12 hours. LC-MS showed 4, 6-difluoro-3-[7-(5-fluoro-2-pyridyl)-4, 7-diazaspiro [2.5]octan-4-yl]-1H-indazole was consumed completely and desired mass was detected. The reaction mixture was concentrated under reduced pressure to give a residue. The residue was purified by prep-HPLC (neutral condition, column: Waters Xbridge Prep OBD C18 150*40 mm*10 um; mobile phase: [water(NH₄HCO₃)-ACN];B %: 50%-80%, 8 mins) to give desired 4, 6-difluoro-3-(7-(5-fluoropyridin-2-yl)-4, 7-diazaspiro [2.5]octan-4-yl)-1-((1-(isopropylsulfonyl)-1H-pyrrol-3-yl) sulfonyl)-1H-indazole (24.8 mg, 41.4 μmol, 24.8% yield, 99.30% purity) as a white solid. ¹H NMR (400 MHz, DMSO-d₆) δ 8.05 (d, J=3.1 Hz, 1H), 7.97 (s, 1H), 7.67-7.62 (m, 1H), 7.49 (dt, J=3.1, 8.8 Hz, 1H), 7.35-7.26 (m, 2H), 6.84 (dd, J=3.3, 9.2 Hz, 1H), 6.53-6.48 (m, 1H), 3.94-3.84 (m, 1H), 3.68-3.62 (m, 2H), 3.59 (s, 2H), 3.43-3.33 (m, 2H), 1.02 (d, J=6.8 Hz, 6H), 0.90-0.80 (m, 2H), 0.58 (s, 2H). HPLC: 99.30% (220 nm), 99.18% (215 nm), 99.48% (254 nm). MS (ESI): mass calcd. For C₂₅H₂₅F₃N₆O₄S₂ 594.13 m/z found 595.2 [M+H]⁺.

### Compound 166: 4, 6-difluoro-3-(7-(5-fluoropyrimidin-2-yl)-4, 7-diazaspiro [2.5]octan-4-yl)-1-((1-(isopropylsulfonyl)-1H-pyrrol-3-yl) sulfonyl)-1H-indazole

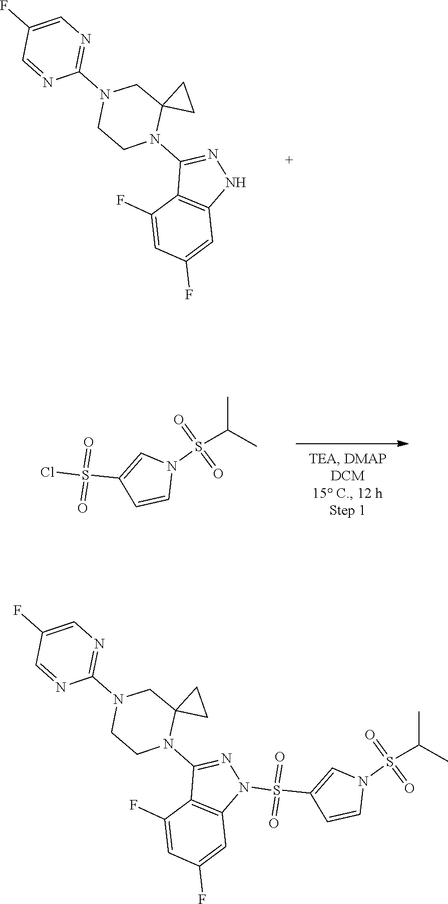 [SMILES: Fc1cnc(N2CCN(c3n[nH]c4cc(F)cc(F)c34)C3(CC3)C2)nc1 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cn4)CC34CC4)c3c(F)cc(F)cc32)c1]

Step 1: To a solution of 4,6-difluoro-3-[7-(5-fluoro-2-pyridyl)-4,7-diazaspiro[2.5]octan-4-yl]-1H-indazole (60 mg, 167 μmol, 1 eq) and 1-isopropylsulfonylpyrrole-3-sulfonyl chloride (68.1 mg, 250 μmol, 1.5 eq) in DCM (1 mL) was added TEA (33.8 mg, 334.0 μmol, 46.5 μL, 2 eq) and DMAP (2.04 mg, 16.7 μmol, 0.1 eq). The mixture was stirred at 15° C. for 12 hours. LC-MS showed 4, 6-difluoro-3-[7-(5-fluoro-2-pyridyl)-4, 7-diazaspiro [2.5]octan-4-yl]-1H-indazole (60 mg, 167 μmol, 1 eq) was consumed completely and desired mass was detected. The reaction mixture was concentrated under reduced pressure to give a residue. The residue was purified by prep-HPLC (neutral condition, column: Waters Xbridge Prep OBD C18 150*40 mm*10 um; mobile phase: [water(NH₄HCO₃)-ACN]; B %: 50%-80%, 8 mins) to give desired 4, 6-difluoro-3-(7-(5-fluoropyridin-2-yl)-4, 7-diazaspiro [2.5]octan-4-yl)-1-((1-(isopropylsulfonyl)-1H-pyrrol-3-yl) sulfonyl)-1H-indazole (24.8 mg, 41.4 μmol, 24.8% yield, 99.30% purity) as a white solid. ¹H NMR (400 MHz, DMSO-d₆) δ 8.05 (d, J=3.1 Hz, 1H), 7.97 (s, 1H), 7.67-7.62 (m, 1H), 7.49 (dt, J=3.1, 8.8 Hz, 1H), 7.35-7.26 (m, 2H), 6.84 (dd, J=3.3, 9.2 Hz, 1H), 6.53-6.48 (m, 1H), 3.94-3.84 (m, 1H), 3.68-3.62 (m, 2H), 3.59 (s, 2H), 3.43-3.33 (m, 2H), 1.02 (d, J=6.8 Hz, 6H), 0.90-0.80 (m, 2H), 0.58 (s, 2H). HPLC: 99.30% (220 nm), 99.18% (215 nm), 99.48% (254 nm). MS (ESI): mass calcd. For C₂₅H₂₅F₃N₆O₄S₂ 594.13 m/z found 595.2 [M+H]⁺.

### Compound 167: 6-chloro-4-fluoro-3-(7-(5-fluoropyrimidin-2-yl)-4, 7-diazaspiro [2.5]octan-4-yl)-1-((1-(isopropylsulfonyl)-1H-pyrrol-3-yl) sulfonyl)-1H-indazole

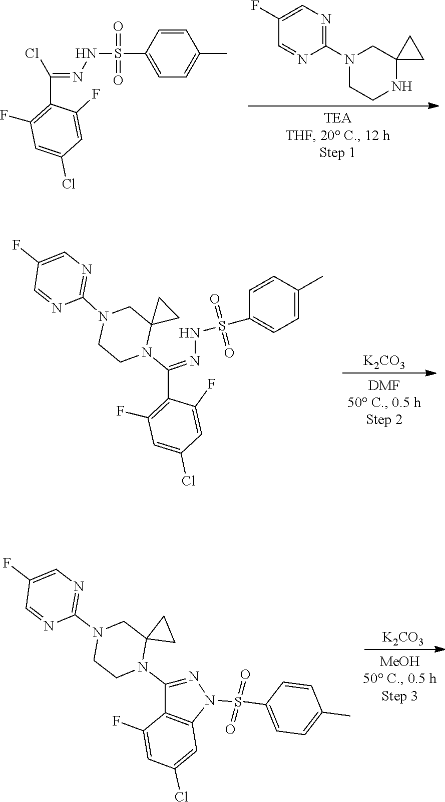

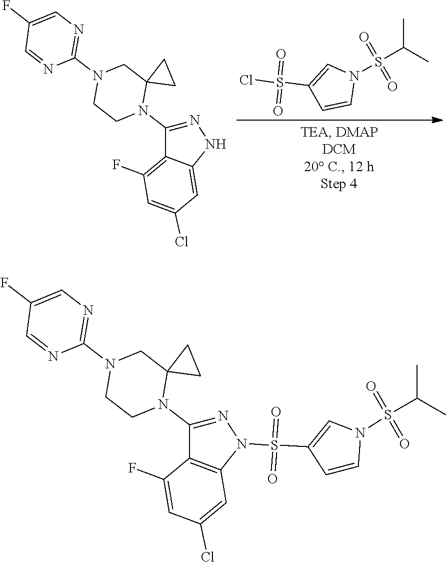 [SMILES: Fc1cnc(N2CCN(c3n[nH]c4cc(Cl)cc(F)c34)C3(CC3)C2)nc1 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cn4)CC34CC4)c3c(F)cc(Cl)cc32)c1]

Step 1: The reaction mixture of 7-(5-fluoropyrimidin-2-yl)-4,7-diazaspiro[2.5]octane (200 mg, 817 μmol, 1 eq, HCl) and TEA (248 mg, 2.45 mmol, 3 eq) in THF (3 mL) was stirred at 20° C. for 0.5 hour. To the mixture was added the solution of (1Z)-4-chloro-2,6-difluoro-N-(p-tolylsulfonyl)benzohydrazonoyl chloride (345 mg, 910 μmol, 1.11 eq) in THF (4 mL) and the reaction mixture was stirred at 20° C. for 11.5 hours. LC-MS showed 7-(5-fluoropyrimidin-2-yl)-4, 7-diazaspiro [2.5]octane remained and desired mass was detected. The reaction mixture was concentrated to give desired N—[(Z)-[(4-chloro-2, 6-difluoro-phenyl)-[7-(5-fluoropyrimidin-2-yl)-4, 7-diazaspiro [2.5]octan-4-yl]methylene]amino]-4-methyl-benzenesulfonamide (450 mg, crude) as a yellow solid. MS (ESI): mass calcd. For C₂₄H₂₂ClF₃N₆O₂S 550.12 m/z found 551.1 [M+H]⁺.

Step 2: The reaction mixture of N—[(Z)-[(4-chloro-2,6-difluoro-phenyl)-[7-(5-fluoropyrimidin-2-yl)-4,7-diazaspiro[2.5]octan-4-yl]methylene]amino]-4-methyl-benzenesulfonamide (450 mg, 816 μmol, 1 eq) and K₂CO₃ (1.13 g, 8.17 mmol, 10 eq) in DMF (4 mL) was stirred at 50° C. for 0.5 hour. LCMS showed N—[(Z)-[(4-chloro-2, 6-difluoro-phenyl)-[7-(5-fluoropyrimidin-2-yl)-4, 7-diazaspiro [2.5]octan-4-yl]methylene]amino]-4-methyl-benzenesulfonamide remained and desired mass was detected. The solution was added water (10 mL) and extracted with MTBE (2*10 mL). The combined organics were washed with brine (10 mL) and concentrated to give desired 6-chloro-4-fluoro-3-[7-(5-fluoropyrimidin-2-yl)-4,7-diazaspiro[2.5]octan-4-yl]-1-(p-tolylsulfonyl) indazole (430 mg, crude) as a yellow solid. MS (ESI): mass calcd. For C₂₄H₂₁ClF₂N₆O₂S 530.11 m/z found 531.1 [M+H]⁺.

Step 3: The reaction mixture of 6-chloro-4-fluoro-3-[7-(5-fluoropyrimidin-2-yl)-4, 7-diazaspiro [2.5]octan-4-yl]-1-(p-tolylsulfonyl) indazole (430 mg, 810 μmol, 1 eq) and K₂CO₃ (560 mg, 4.05 mmol, 5 eq) in MeOH (4 mL) was stirred at 50° C. for 0.5 hour. LCMS showed 6-chloro-4-fluoro-3-[7-(5-fluoropyrimidin-2-yl)-4, 7-diazaspiro [2.5]octan-4-yl]-1-(p-tolylsulfonyl) indazole was consumed completely and desired mass was detected. The reaction mixture was concentrated to get a residue. The residue was purified by prep-TLC (SiO₂, petroleum ether/ethyl acetate=1/1) to give desired 6-chloro-4-fluoro-3-[7-(5-fluoropyrimidin-2-yl)-4, 7-diazaspiro [2.5]octan-4-yl]-1H-indazole (104 mg, crude) as a white solid. MS (ESI): mass calcd. For C₁₇H₁₅ClF₂N₆ 376.10 m/z found 377.1 [M+H]⁺.

Step 4: To the solution of 6-chloro-4-fluoro-3-[7-(5-fluoropyrimidin-2-yl)-4,7-diazaspiro[2.5]octan-4-yl]-1H-indazole (50 mg, 132 μmol, 1 eq), TEA (53.7 mg, 531 μmol, 4 eq) and DMAP (1.62 mg, 13.3 μmol, 0.1 eq) in DCM (3 mL) was added 1-isopropylsulfonylpyrrole-3-sulfonyl chloride (54.1 mg, 199 μmol, 1.5 eq) at 20° C. and the solution was stirred at 20° C. for 12 hours. LCMS showed 6-chloro-4-fluoro-3-[7-(5-fluoropyrimidin-2-yl)-4, 7-diazaspiro [2.5]octan-4-yl]-1H-indazole remained and desired mass was detected. The reaction was concentrated to get a residue. The residue was purified by prep-HPLC (column: Waters Xbridge BEH C18 100*30 mm*10 um; mobile phase: [water(NH₄HCO₃)-ACN]; B %: 60%-90%, 8 min) to give desired 6-chloro-4-fluoro-3-[7-(5-fluoropyrimidin-2-yl)-4,7-diazaspiro[2.5]octan-4-yl]-1-(1-isopropylsulfonylpyrrol-3-yl)sulfonyl-indazole (23.6 mg, 38.5 μmol, 29.4% yield, 99.75% purity) as a white solid. ¹H NMR (400 MHz, DMSO-d₆) δ 8.45 (s, 2H), 8.03 (s, 1H), 7.89 (d, 1H), 7.46 (d, 1H), 7.34 (m, 1H), 6.55 (m, 1H), 3.85-4.01 (m, 3H), 3.63 (br s, 4H), 1.06 (d, 6H), 0.87 (br s, 2H), 0.60 (s, 2H). HPLC: 99.75% (220 nm), 99.70% (215 nm), 99.92% (254 nm). MS (ESI): mass calcd. For C₂₄H₂₄ClF₂N₇O₄S₂ 611.10, m/z found 612.0 [M+H]⁺.

### Compound 168: 6-chloro-3-[7-(3, 5-difluoro-2-pyridyl)-4, 7-diazaspiro [2.5]octan-4-yl]-4-fluoro-1-(1-isopropylsulfonylpyrrol-3-yl) sulfonyl-indazole

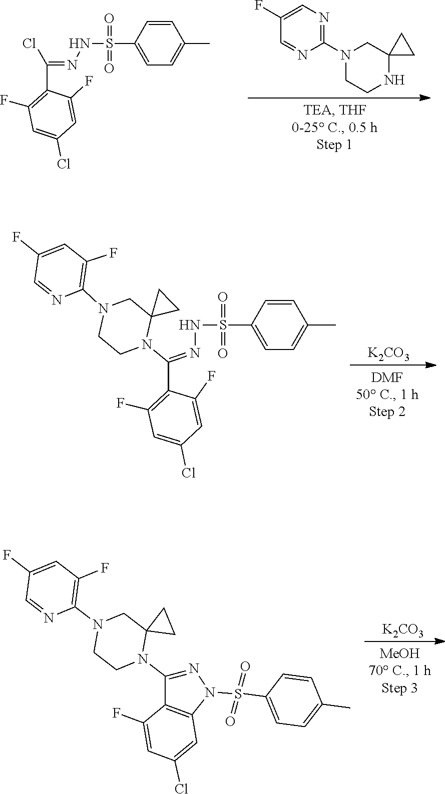

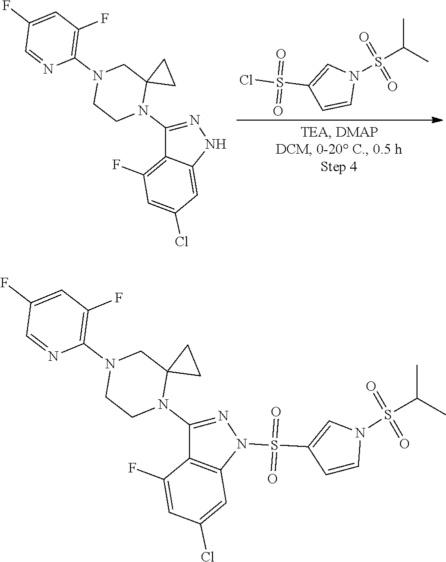 [SMILES: Fc1cnc(N2CCN(c3n[nH]c4cc(Cl)cc(F)c34)C3(CC3)C2)c(F)c1 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cc4F)CC34CC4)c3c(F)cc(Cl)cc32)c1]

Step 1: To a solution of 7-(3, 5-difluoro-2-pyridyl)-4, 7-diazaspiro [2.5]octane (216 mg, 827 μmol, 1 eq, HCl) in THF (10 mL) was added dropwise TEA (418 mg, 4.14 mmol, 576 μL, 5 eq) at 25° C. After addition, the mixture was stirred at this temperature for 10 mins, and then (1Z)-4-chloro-2, 6-difluoro-N-(p-tolylsulfonyl)benzohydrazonoyl chloride (345 mg, 910 μmol, 1.1 eq) in was added dropwise at 0° C. The resulting mixture was stirred at 25° C. for 20 mins. LC-MS showed 7-(3, 5-difluoro-2-pyridyl)-4, 7-diazaspiro [2.5]octane was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(Z)-[(4-chloro-2, 6-difluoro-phenyl)-[7-(3, 5-difluoro-2-pyridyl)-4, 7-diazaspiro [2.5]octan-4-yl]methylene]amino]-4-methyl-benzenesulfonamide (450 mg, crude) as a yellow solid. MS (ESI): mass calcd. For C₂₅H₂₂ClF₄N₅O₂S 567.11, m/z found 568.2 [M+H]⁺.

Step 2: To a solution of N—[(Z)-[(4-chloro-2, 6-difluoro-phenyl)-[7-(3, 5-difluoro-2-pyridyl)-4, 7-diazaspiro [2.5]octan-4-yl]methylene]amino]-4-methyl-benzenesulfonamide (450 mg, 792 μmol, 1 eq) in DMF (5 mL) was added K₂CO₃ (548 mg, 3.96 mmol, 5 eq). The mixture was stirred at 50° C. for 1 hour. LC-MS showed N—[(Z)-[(4-chloro-2, 6-difluoro-phenyl)-[7-(3, 5-difluoro-2-pyridyl)-4, 7-diazaspiro [2.5]octan-4-yl]methylene]amino]-4-methyl-benzenesulfonamide was consumed completely and desired compound was detected. Then it was separated between 50 mL of water and 100 mL of ethyl acetate. The organic phase was separated, washed with 50 mL of brine, dried over Na₂SO₄, filtered and concentrated under reduced pressure to give desired 6-chloro-3-[7-(3, 5-difluoro-2-pyridyl)-4, 7-diazaspiro [2.5]octan-4-yl]-4-fluoro-1-(p-tolylsulfonyl) indazole (430 mg, crude) as a black oil. MS (ESI): mass calcd. For C₂₅H₂₁ClF₃N₅O₂S 547.11, m/z found 548.2 [M+H]⁺.

Step 3: To a solution of 6-chloro-3-[7-(3, 5-difluoro-2-pyridyl)-4, 7-diazaspiro [2.5]octan-4-yl]-4-fluoro-1-(p-tolylsulfonyl) indazole (430 mg, 785 μmol, 1 eq) in MeOH (20 mL) was added K₂CO₃ (542 mg, 3.92 mmol, 5 eq). The mixture was stirred at 70° C. for 1 hour. LC-MS showed 6-chloro-3-[7-(3, 5-difluoro-2-pyridyl)-4, 7-diazaspiro [2.5]octan-4-yl]-4-fluoro-1-(p-tolylsulfonyl) indazole was consumed completely and desired compound was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by column chromatography (SiO₂, petroleum ether/Ethyl acetate=1/0 to 1/1) to give desired 6-chloro-3-[7-(3, 5-difluoro-2-pyridyl)-4, 7-diazaspiro [2.5]octan-4-yl]-4-fluoro-1H-indazole (140 mg, 356 μmol, 45.3% yield) as a yellow oil. MS (ESI): mass calcd. For C₁₈H₁₅ClF₃N₅ 393.10, m/z found 394.2 [M+H]⁺.

Step 4: To a solution of 6-chloro-3-[7-(3, 5-difluoro-2-pyridyl)-4, 7-diazaspiro [2.5]octan-4-yl]-4-fluoro-1H-indazole (50 mg, 127 μmol, 1 eq) and 1-isopropylsulfonylpyrrole-3-sulfonyl chloride (69.0 mg, 254 μmol, 2 eq) in DCM (2 mL) was added TEA (25.7 mg, 254 μmol, 35.4 μL, 2 eq) and DMAP (1.55 mg, 12.7 μmol, 0.1 eq) at 0° C. The mixture was stirred at 20° C. for 0.5 hour. LC-MS showed 6-chloro-3-[7-(3, 5-difluoro-2-pyridyl)-4, 7-diazaspiro [2.5]octan-4-yl]-4-fluoro-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Waters Xbridge BEH C18 100*30 mm*10 um; mobile phase: [water(NH₄HCO₃)-ACN]; B %: 50%-90%, 8 min) to give desired 6-chloro-3-[7-(3, 5-difluoro-2-pyridyl)-4, 7-diazaspiro [2.5]octan-4-yl]-4-fluoro-1-(1-isopropylsulfonylpyrrol-3-yl) sulfonyl-indazole (10.6 mg, 16.9 μmol, 13.3% yield, 100.00% purity) as a yellow solid. ¹H NMR (400 MHz, DMSO-d₆) δ 8.13-8.06 (m, 1H), 8.01 (br d, J=1.6 Hz, 1H), 7.89 (s, 1H), 7.80 (ddd, J=2.3, 8.3, 12.5 Hz, 1H), 7.45 (br d, J=10.1 Hz, 1H), 7.37-7.32 (m, 1H), 6.55 (br d, J=1.5 Hz, 1H), 3.98-3.88 (m, 1H), 3.73 (br s, 2H), 3.33 (d, J=2.3 Hz, 3H), 3.27 (br s, 1H), 1.08 (dd, J=1.8, 6.7 Hz, 6H), 0.87 (br s, 2H), 0.62 (br s, 2H). HPLC: 100.00% (220 nm), 99.96% (215 nm), 100.00% (254 nm). MS (ESI): mass calcd. For C₂₅H₂₄ClF₃N₆O₄S₂ 628.09, m/z found 629.2 [M+H]⁺.

### Compound 169: 3-[4, 6-difluoro-3-[7-(5-fluoropyrimidin-2-yl)-4, 7-diazaspiro [2.5]octan-4-yl]indazol-1-yl]sulfonyl-N, N-dimethyl-pyrrole-1-sulfonamide

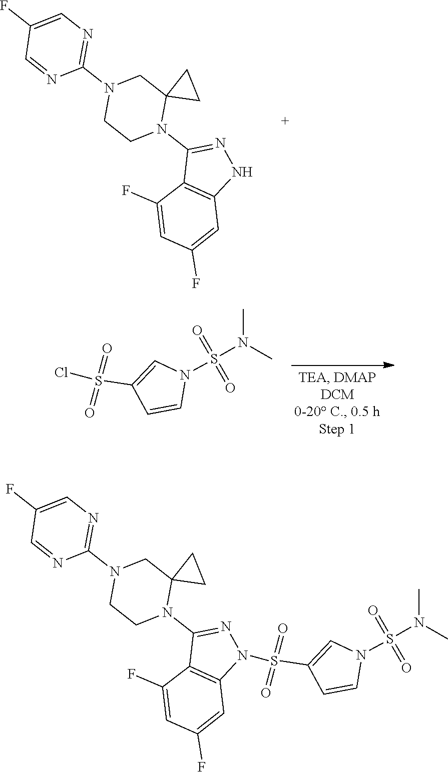 [SMILES: Fc1cnc(N2CCN(c3n[nH]c4cc(F)cc(F)c34)C3(CC3)C2)nc1 ; CN(C)S(=O)(=O)n1ccc(S(=O)(=O)Cl)c1 ; CN(C)S(=O)(=O)n1ccc(S(=O)(=O)n2nc(N3CCN(c4ncc(F)cn4)CC34CC4)c3c(F)cc(F)cc32)c1]

Step 1: To a solution of 4, 6-difluoro-3-[7-(5-fluoropyrimidin-2-yl)-4, 7-diazaspiro [2.5]octan-4-yl]-1H-indazole (20 mg, 55.5 μmol, 1 eq) and 1-(dimethylsulfamoyl)pyrrole-3-sulfonyl chloride (30.3 mg, 111 μmol, 2 eq) in DCM (2 mL) was added TEA (11.2 mg, 111 μmol, 15.5 μL, 2 eq) and DMAP (678 μg, 5.55 μmol, 0.1 eq) at 0° C. The mixture was stirred at 20° C. for 0.5 hour. LC-MS showed 4, 6-difluoro-3-[7-(5-fluoropyrimidin-2-yl)-4, 7-diazaspiro [2.5]octan-4-yl]-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-TLC (SiO₂, petroleum ether/Ethyl acetate=2/1) to give desired 3-[4,6-difluoro-3-[7-(5-fluoropyrimidin-2-yl)-4,7-diazaspiro[2.5]octan-4-yl]indazol-1-yl]sulfonyl-N, N-dimethyl-pyrrole-1-sulfonamide (3.0 mg, 4.80 μmol, 8.65% yield, 95.47% purity) as a white solid. ¹H NMR (400 MHz, DMSO-d₆) δ 8.51 (s, 2H), 8.05 (t, J=1.9 Hz, 1H), 7.74 (dd, J=1.8, 8.9 Hz, 1H), 7.44-7.34 (m, 2H), 6.57 (dd, J=1.6, 3.3 Hz, 1H), 3.96 (s, 2H), 3.70 (br s, 4H), 2.75 (s, 6H), 0.95-0.91 (m, 2H), 0.65 (s, 2H). HPLC: 95.47% (220 nm), 95.06% (215 nm), 95.99% (254 nm). MS (ESI): mass calcd. For C₂₃H₂₃F₃N₈O₄S₂ 596.12, m/z found 597.2 [M+H]⁺.

### Compound 170: 3-[4-chloro-6-fluoro-3-[7-(5-fluoropyrimidin-2-yl)-4, 7-diazaspiro [2.5]octan-4-yl]indazol-1-yl]sulfonyl-N, N-dimethyl-pyrrole-1-sulfonamide

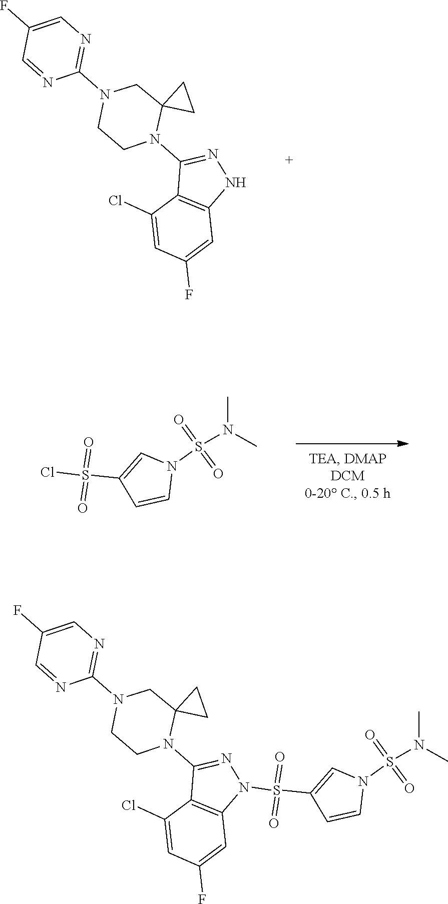 [SMILES: Fc1cnc(N2CCN(c3n[nH]c4cc(F)cc(Cl)c34)C3(CC3)C2)nc1 ; CN(C)S(=O)(=O)n1ccc(S(=O)(=O)Cl)c1 ; CN(C)S(=O)(=O)n1ccc(S(=O)(=O)n2nc(N3CCN(c4ncc(F)cn4)CC34CC4)c3c(Cl)cc(F)cc32)c1]

Step 1: To a solution of 4-chloro-6-fluoro-3-[7-(5-fluoropyrimidin-2-yl)-4, 7-diazaspiro [2.5]octan-4-yl]-1H-indazole (20 mg, 53.1 μmol, 1 eq) and 1-(dimethylsulfamoyl)pyrrole-3-sulfonyl chloride (29.0 mg, 106 μmol, 2 eq) in DCM (2 mL) was added TEA (10.7 mg, 106 μmol, 14.8 μL, 2 eq) and DMAP (648 μg, 5.31 μmol, 0.1 eq) at 0° C. The mixture was stirred at 20° C. for 0.5 hour. LC-MS showed 4-chloro-6-fluoro-3-[7-(5-fluoropyrimidin-2-yl)-4, 7-diazaspiro [2.5]octan-4-yl]-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Waters Xbridge Prep OBD C18 150*40 mm*10 um; mobile phase: [water(NH₄HCO₃)-ACN]; B %: 45%-85%, 8 min) to give desired 3-[4-chloro-6-fluoro-3-[7-(5-fluoropyrimidin-2-yl)-4, 7-diazaspiro [2.5]octan-4-yl]indazol-1-yl]sulfonyl-N, N-dimethyl-pyrrole-1-sulfonamide (13.1 mg, 21.4 μmol, 40.2% yield, 99.91% purity) as a white solid. ¹H NMR (400 MHz, DMSO-d₆) δ 8.52 (s, 2H), 8.09 (s, 1H), 7.87 (dd, J=2.1, 8.9 Hz, 1H), 7.58 (dd, J=2.1, 9.1 Hz, 1H), 7.40 (dd, J=2.5, 3.1 Hz, 1H), 6.60 (dd, J=1.6, 3.4 Hz, 1H), 3.99 (s, 2H), 3.75 (br s, 2H), 3.63-3.57 (m, 2H), 2.76 (s, 6H), 0.84 (s, 2H), 0.53 (s, 2H). HPLC: 99.91% (220 nm), 99.98% (215 nm), 100.00% (254 nm). MS (ESI): mass calcd. For C₂₃H₂₃ClF₂N₈O₄S₂ 612.09, m/z found 613.2 [M+H]⁺.

### Compound 171: 1-((1-(tert-butylsulfonyl)-1H-pyrrol-3-yl) sulfonyl)-4-chloro-6-fluoro-3-(7-(5-fluoropyrimidin-2-yl)-4, 7-diazaspiro [2.5]octan-4-yl)-1H-indazole

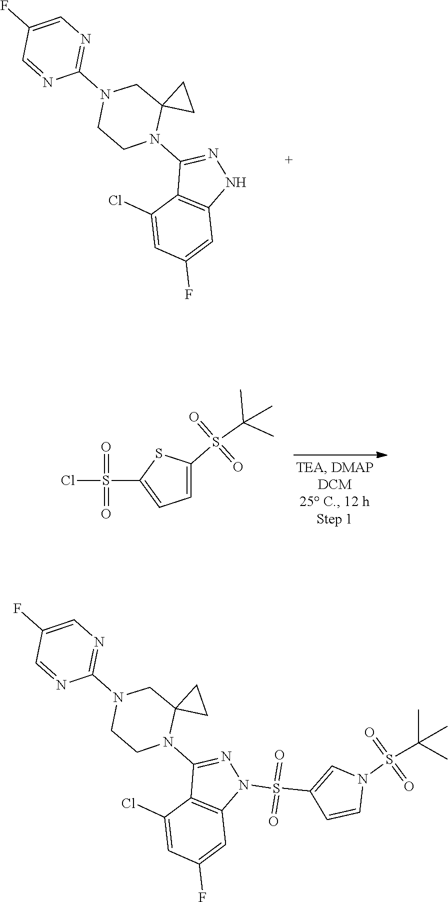 [SMILES: Fc1cnc(N2CCN(c3n[nH]c4cc(F)cc(Cl)c34)C3(CC3)C2)nc1 ; CC(C)(C)S(=O)(=O)c1ccc(S(=O)(=O)Cl)s1 ; CC(C)(C)S(=O)(=O)n1ccc(S(=O)(=O)n2nc(N3CCN(c4ncc(F)cn4)CC34CC4)c3c(Cl)cc(F)cc32)c1]

Step 1: To a solution of 4-chloro-6-fluoro-3-[7-(5-fluoropyrimidin-2-yl)-4,7-diazaspiro[2.5]octan-4-yl]-1H-indazole (25 mg, 66.4 μmol, 1 eq) and 1-tert-butylsulfonylpyrrole-3-sulfonyl chloride (28.4 mg, 99.5 μmol, 1.5 eq) in DCM (1 mL) was added TEA (13.4 mg, 133 μmol, 18.5 μL, 2 eq) and DMAP (811 μg, 6.63 μmol, 0.1 eq). The mixture was stirred at 25° C. for 12 hours. LC-MS showed 4-chloro-6-fluoro-3-[7-(5-fluoropyrimidin-2-yl)-4, 7-diazaspiro [2.5]octan-4-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give a residue. The residue was purified by prep-HPLC (FA condition; column: column: Phenomenex Luna C18 75*30 mm*3 um; mobile phase: [water (FA)-ACN]; B %: 40%-90%, 8 min.) to give desired 1-(1-tert-butylsulfonylpyrrol-3-yl) sulfonyl-4-chloro-6-fluoro-3-[7-(5-fluoropyrimidin-2-yl)-4, 7-diazaspiro [2.5]octan-4-yl]indazole (36.6 mg, 58.5 μmol, 44.1% yield, 99.99% purity) as a white solid. ¹H NMR (400 MHz, DMSO-d₆) δ 8.46 (s, 2H), 8.03-8.01 (m, 1H), 7.85-7.80 (m, 1H), 7.56-7.50 (m, 1H), 6.60-6.58 (m, 1H), 3.97-3.88 (m, 2H), 3.78-3.60 (m, 2H), 3.56-3.50 (m, 2H), 1.27-1.12 (m, 10H), 0.78 (s, 2H), 0.47 (s, 2H). HPLC: 99.99% (220 nm), 99.98% (215 nm), 100.0% (254 nm). MS (ESI): mass calcd. For C₂₅H₂₆ClF₂N₇O₄S₂ 625.11, m/z found 626.1 [M+H]⁺.

### Compound 172: 1-((1-(tert-butylsulfonyl)-1H-pyrrol-3-yl) sulfonyl)-4, 6-difluoro-3-(7-(5-fluoropyrimidin-2-yl)-4, 7-diazaspiro [2.5]octan-4-yl)-1H-indazole

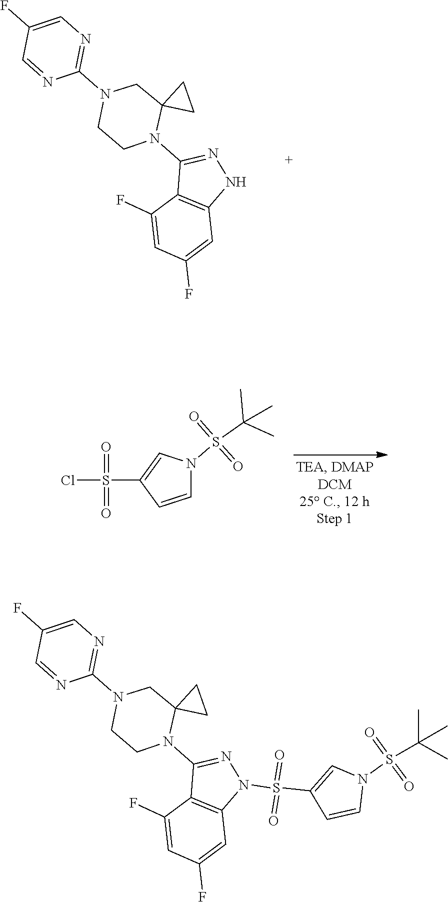 [SMILES: Fc1cnc(N2CCN(c3n[nH]c4cc(F)cc(F)c34)C3(CC3)C2)nc1 ; CC(C)(C)S(=O)(=O)n1ccc(S(=O)(=O)Cl)c1 ; CC(C)(C)S(=O)(=O)n1ccc(S(=O)(=O)n2nc(N3CCN(c4ncc(F)cn4)CC34CC4)c3c(F)cc(F)cc32)c1]

Step 1: To a solution of 4,6-difluoro-3-[7-(5-fluoropyrimidin-2-yl)-4, 7-diazaspiro [2.5]octan-4-yl]-1H-indazole (20 mg, 55.5 μmol, 1 eq) and 1-tert-butylsulfonylpyrrole-3-sulfonyl chloride (19.0 mg, 66.6 μmol, 1.2 eq) in DCM (1 mL) was added TEA (11.2 mg, 111 μmol, 15.5 μL, 2 eq) and DMAP (678 μg, 5.55 μmol, 0.1 eq). The mixture was stirred at 25° C. for 12 hours. LC-MS showed 4, 6-difluoro-3-[7-(5-fluoropyrimidin-2-yl)-4, 7-diazaspiro [2.5]octan-4-yl]-1H-indazole was consumed completely and desired mass was detected. The reaction mixture was partitioned between 10 mL of H₂O and 10 mL of DCM. The organic phase was separated, washed with 5 mL of brine, dried over Na₂SO₄, filtered and concentrated under reduced pressure to give a residue. The residue was purified by prep-HPLC (FA condition; column: Phenomenex Luna C18 75*30 mm*3 um; mobile phase: [water (FA)-ACN]; B %: 60%-80%, 8 min) to give desired 1-(1-tert-butylsulfonylpyrrol-3-yl) sulfonyl-4, 6-difluoro-3-[7-(5-fluoropyrimidin-2-yl)-4, 7-diazaspiro [2.5]octan-4-yl]indazole (11.3 mg, 18.4 μmol, 33.2% yield, 99.42% purity) as a white solid. ¹H NMR (400 MHz, DMSO-d₆) δ 8.48-8.44 (m, 2H), 7.98-7.95 (m, 1H), 7.72-7.67 (m, 1H), 7.38-7.31 (m, 2H), 6.56 (dd, J=1.6, 3.4 Hz, 1H), 3.93-3.87 (m, 2H), 3.70-3.57 (m, 4H), 1.18 (s, 9H), 0.90-0.83 (m, 2H), 0.63-0.56 (m, 2H). HPLC: 99.42% (220 nm), 99.75% (215 nm), 100.0% (254 nm). MS (ESI): mass calcd. For C₂₅H₂₆F₃N₇O₄S₂609.14, m/z found 610.1 [M+H]⁺.

### Compound 173: 4-chloro-1-((1-(cyclopropylsulfonyl)-1H-pyrrol-3-yl) sulfonyl)-6-fluoro-3-(7-(5-fluoropyrimidin-2-yl)-4, 7-diazaspiro [2.5]octan-4-yl)-1H-indazole

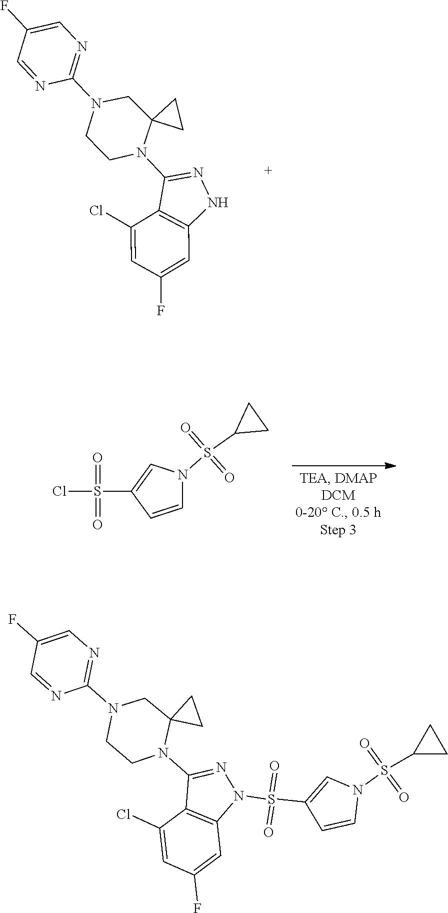 [SMILES: Fc1cnc(N2CCN(c3n[nH]c4cc(F)cc(Cl)c34)C3(CC3)C2)nc1 ; O=S(=O)(Cl)c1ccn(S(=O)(=O)C2CC2)c1 ; O=S(=O)(c1ccn(S(=O)(=O)C2CC2)c1)n1nc(N2CCN(c3ncc(F)cn3)CC23CC3)c2c(Cl)cc(F)cc21]

Step 1: To a solution of 4-chloro-6-fluoro-3-[7-(5-fluoropyrimidin-2-yl)-4, 7-diazaspiro [2.5]octan-4-yl]-1H-indazole (20.9 mg, 55.6 μmol, 1 eq) and 1-cyclopropylsulfonylpyrrole-3-sulfonyl chloride (30 mg, 111 μmol, 2 eq) in DCM (2 mL) was added TEA (11.3 mg, 111 μmol, 15.5 μL, 2 eq) and DMAP (679 μg, 5.56 μmol, 0.1 eq) at 0° C. The mixture was stirred at 20° C. for 0.5 hour. LC-MS showed 1-cyclopropylsulfonylpyrrole-3-sulfonyl chloride was consumed completely and one main peak with desired mass was detected. The reaction mixture was filtered and the filter liquor was concentrated under reduced pressure to give a residue. The residue was purified by prep-HPLC (basic condition; column: Waters Xbridge BEH C18 100*30 mm*10 um; mobile phase: [water(NH₄HCO₃)-ACN]; B %: 62%-92%, 8 min) to give desired 4-chloro-1-(1-cyclopropylsulfonylpyrrol-3-yl)sulfonyl-6-fluoro-3-[7-(5-fluoropyrimidin-2-yl)-4, 7-diazaspiro [2.5]octan-4-yl]indazole (3 mg, 4.68 μmol, 8.42% yield, 95.17% purity) as a white solid. ¹H NMR (400 MHz, DMSO-d₆) δ 8.45 (s, 2H), 8.09 (s, 1H), 7.79 (br d, J=7.8 Hz, 1H), 7.53 (br d, J=8.8 Hz, 1H), 7.37 (br s, 1H), 6.54 (br d, J=1.3 Hz, 1H), 3.92 (s, 2H), 3.68 (br s, 2H), 3.53 (br s, 2H), 3.26-3.20 (m, 1H), 1.29 (br d, J=2.6 Hz, 2H), 1.13 (br d, J=5.8 Hz, 2H), 0.77 (br s, 2H), 0.46 (br s, 2H). HPLC: 95.17% (220 nm), 94.16% (215 nm), 97.24% (254 nm) MS (ESI): mass calcd. For C₂₄H₂₂N₇S₂O₄ClF₂ 609.08, m/z found 610.2 [M+H]⁺.

### Compound 174: 1-((1-(cyclopropylsulfonyl)-1H-pyrrol-3-yl) sulfonyl)-4, 6-difluoro-3-(7-(5-fluoropyrimidin-2-yl)-4, 7-diazaspiro [2.5]octan-4-yl)-1H-indazole

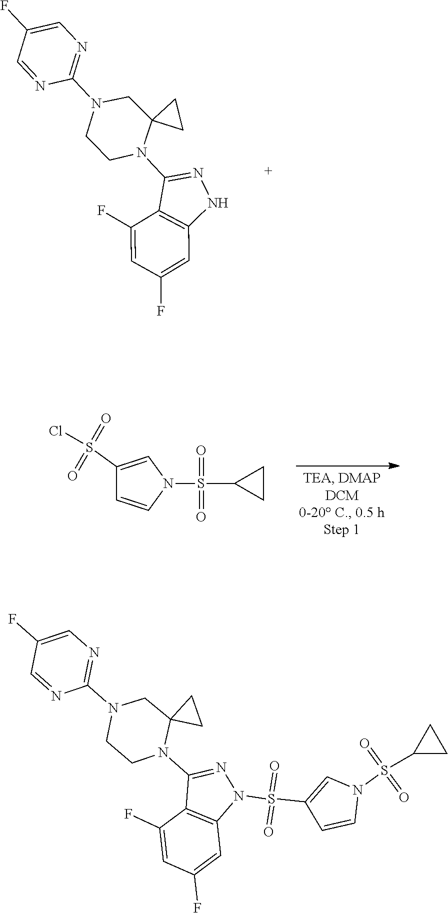 [SMILES: Fc1cnc(N2CCN(c3n[nH]c4cc(F)cc(F)c34)C3(CC3)C2)nc1 ; O=S(=O)(Cl)c1ccn(S(=O)(=O)C2CC2)c1 ; O=S(=O)(c1ccn(S(=O)(=O)C2CC2)c1)n1nc(N2CCN(c3ncc(F)cn3)CC23CC3)c2c(F)cc(F)cc21]

Step 1: To a solution of 1-cyclopropylsulfonylpyrrole-3-sulfonyl chloride (30 mg, 111 μmol, 2 eq) and 4,6-difluoro-3-[7-(5-fluoropyrimidin-2-yl)-4, 7-diazaspiro [2.5]octan-4-yl]-1H-indazole (20.0 mg, 55.6 μmol, 1 eq) in DCM (2 mL) was added TEA (11.3 mg, 111 μmol, 15.5 μL, 2 eq) and DMAP (679 μg, 5.56 μmol, 0.1 eq) at 0° C. The mixture was stirred at 20° C. for 0.5 hour. LC-MS showed 1-cyclopropylsulfonylpyrrole-3-sulfonyl chloride was consumed completely and desired mass was detected. The reaction mixture was filtered and the filter liquor was concentrated under reduced pressure to give a residue. The residue was purified by prep-HPLC (basic condition; Waters Xbridge BEH C18 100*30 mm*10 um; mobile phase:[water(NH₄HCO₃)-ACN]; B %: 50%-80%, 8 min) to give desired 1-(1-cyclopropylsulfonylpyrrol-3-yl)sulfonyl-4,6-difluoro-3-[7-(5-fluoropyrimidin-2-yl)-4, 7-diazaspiro [2.5]octan-4-yl]indazole (3 mg, 4.89 μmol, 8.79% yield, 96.67% purity) as a white solid. ¹H NMR (400 MHz, DMSO-d₆) δ 8.46 (s, 2H), 8.07 (s, 1H), 7.68 (s, 1H), 7.38 (br d, J=2.5 Hz, 2H), 6.59-6.47 (m, 1H), 3.91 (s, 2H), 3.65 (br s, 4H), 3.28-3.22 (m, 1H), 1.34-1.28 (m, 2H), 1.14 (dd, J=2.3, 7.7 Hz, 2H), 0.87 (s, 2H), 0.59 (s, 2H). HPLC: 96.67% (220 nm), 94.59% (215 nm), 98.75% (254 nm). MS (ESI): mass calcd. For C₂₄H₂₂N₇S₂O₄F₃ 593.60, m/z found 594.2 [M+H]⁺.

### Compound 175: 4-chloro-6-fluoro-3-(7-(4-fluorophenyl)-4, 7-diazaspiro [2.5]octan-4-yl)-1-((1-(isopropylsulfonyl)-1H-pyrrol-3-yl) sulfonyl)-1H-indazole

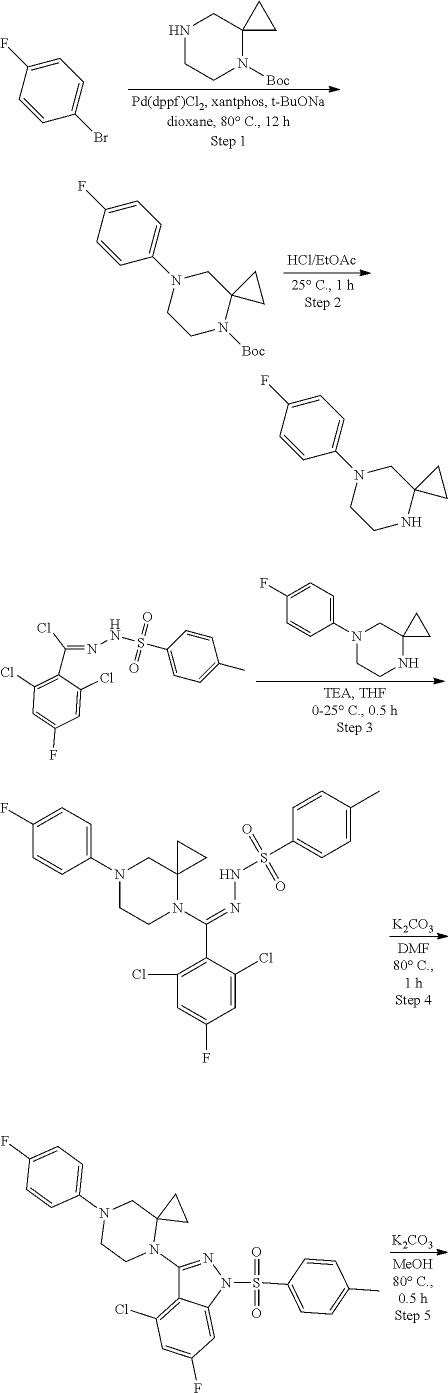

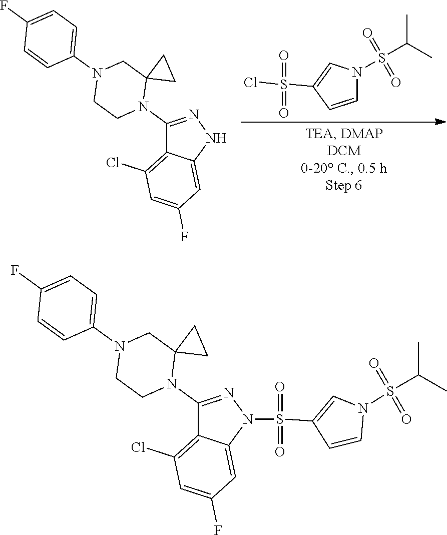 [SMILES: Fc1ccc(N2CCN(c3n[nH]c4cc(F)cc(Cl)c34)C3(CC3)C2)cc1 ; CC(C)S(=O)(=O)n1ccc(S(=O)(=O)Cl)c1 ; CC(C)S(=O)(=O)n1ccc(S(=O)(=O)n2nc(N3CCN(c4ccc(F)cc4)CC34CC4)c3c(Cl)cc(F)cc32)c1]

Step 1: A mixture of 1-bromo-4-fluoro-benzene (500 mg, 2.86 mmol, 314 μL, 1 eq), tert-butyl 4, 7-diazaspiro [2.5]octane-4-carboxylate (607 mg, 2.86 mmol, 1 eq), Pd(dppf)Cl₂ (209 mg, 286 μmol, 0.1 eq), Xantphos (331 mg, 571 μmol, 0.2 eq) and t-BuONa (824 mg, 8.57 mmol, 3 eq) in dioxane (10 mL) was degassed and purged with N₂ for 3 times, and then the mixture was stirred at 80° C. for 12 hours under N₂ atmosphere. LC-MS showed 1-bromo-4-fluoro-benzene was consumed completely and desired mass was detected. The reaction mixture was added to 20 mL of water, the aqueous phase was extracted with 60 mL of EtOAc. The organic layer was dried over Na₂SO₄, filtered and the filtrate was concentrated to give a residue. The residue was purified by column chromatography (SiO₂, petroleum ether/Ethyl acetate=0/1 to 1/0) to give desired tert-butyl 7-(4-fluorophenyl)-4, 7-diazaspiro [2.5]octane-4-carboxylate (670 mg, crude) as a yellow solid. MS (ESI): mass calcd. For C₁₇H₂₃FN₂O₂ 306.17, m/z found 307.2 [M+H]⁺.

Step 2: To a solution of tert-butyl 7-(4-fluorophenyl)-4, 7-diazaspiro [2.5]octane-4-carboxylate (670 mg, 2.19 mmol, 1 eq) in HCl/EtOAc (5 mL) was stirred at 25° C. for 1 hour. LC-MS showed tert-butyl 7-(4-fluorophenyl)-4, 7-diazaspiro [2.5]octane-4-carboxylate was consumed completely and desired mass was detected. The reaction mixture was concentrated under reduced pressure to give desired 7-(4-fluorophenyl)-4, 7-diazaspiro [2.5]octane (650 mg, crude, HCl) as a white solid. MS (ESI): mass calcd. For C₁₂H₁₅FN₂ 206.12, m/z found 207.2 [M+H]⁺.

Step 3: To a solution of 7-(4-fluorophenyl)-4, 7-diazaspiro [2.5]octane (419 mg, 1.73 mmol, 1 eq, HCl) in THF (10 mL) was added dropwise TEA (1.75 g, 17.3 mmol, 2.40 mL, 10 eq) at 25° C. After addition, the mixture was stirred at this temperature for 10 mins, and then (1Z)-2, 6-dichloro-4-fluoro-N-(p-tolylsulfonyl)benzohydrazonoyl chloride (750 mg, 1.90 mmol, 1.1 eq) in THF (2 mL) was added dropwise at 0° C. The resulting mixture was stirred at 25° C. for 20 mins. LC-MS showed 7-(4-fluorophenyl)-4, 7-diazaspiro [2.5]octane was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(Z)-[(2, 6-dichloro-4-fluoro-phenyl)-[7-(4-fluorophenyl)-4, 7-diazaspiro [2.5]octan-4-yl]methylene]amino]-4-methyl-benzenesulfonamide (1 g, crude) as a yellow solid. MS (ESI): mass calcd. For C₂₆H₂₄Cl₂F₂N₄O₂S 564.10, m/z found 565.2 [M+H]⁺.

Step 4: To a solution of N—[(Z)-[(2, 6-dichloro-4-fluoro-phenyl)-[7-(4-fluorophenyl)-4, 7-diazaspiro [2.5]octan-4-yl]methylene]amino]-4-methyl-benzenesulfonamide (1 g, 1.77 mmol, 1 eq) in DMF (5 mL) was added K₂CO₃ (1.22 g, 8.84 mmol, 5 eq). The mixture was stirred at 80° C. for 1 hour. LC-MS showed N—[(Z)-[(2, 6-dichloro-4-fluoro-phenyl)-[7-(4-fluorophenyl)-4, 7-diazaspiro [2.5]octan-4-yl]methylene]amino]-4-methyl-benzenesulfonamide was consumed completely and desired mass was detected. The reaction mixture was added to 20 mL of water, the aqueous phase was extracted with 30 mL of EtOAc. The organic layer was dried over Na₂SO₄, filtered and the filtrate was concentrated to give desired 4-chloro-6-fluoro-3-[7-(4-fluorophenyl)-4, 7-diazaspiro [2.5]octan-4-yl]-1-(p-tolylsulfonyl) indazole (1.1 g, crude) as a black oil. MS (ESI): mass calcd. For C₂₆H₂₃ClF₂N₄O₂S 528.12, m/z found 529.2 [M+H]⁺.

Step 5: To a solution of 4-chloro-6-fluoro-3-[7-(4-fluorophenyl)-4, 7-diazaspiro [2.5]octan-4-yl]-1-(p-tolylsulfonyl) indazole (1.1 g, 2.08 mmol, 1 eq) in MeOH (5 mL) was added K₂CO₃ (1.15 g, 8.32 mmol, 4 eq). The mixture was stirred at 80° C. for 0.5 hour. LC-MS showed 4-chloro-6-fluoro-3-[7-(4-fluorophenyl)-4, 7-diazaspiro [2.5]octan-4-yl]-1-(p-tolylsulfonyl) indazole was consumed completely and desired mass was detected. The reaction mixture was added to 20 mL of water, the aqueous phase was extracted with 60 mL of EtOAc. The organic layer was dried over Na₂SO₄, filtered and the filtrate was concentrated to give a residue. The residue was purified by prep-TLC (SiO₂, petroleum ether/Ethyl acetate=1/1) to give desired 4-chloro-6-fluoro-3-[7-(4-fluorophenyl)-4, 7-diazaspiro [2.5]octan-4-yl]-1H-indazole (290 mg, crude) as a yellow solid. MS (ESI): mass calcd. For C₁₈H₁₆ClF₂N₅ 374.11, m/z found 375.1 [M+H]⁺.

Step 6: To a solution of 4-chloro-6-fluoro-3-[7-(4-fluorophenyl)-4, 7-diazaspiro [2.5]octan-4-yl]-1H-indazole (90 mg, 240 μmol, 1 eq) and 1-isopropylsulfonylpyrrole-3-sulfonyl chloride (131 mg, 480 μmol, 2 eq) in DCM (2 mL) was added TEA (48.6 mg, 480 μmol, 66.8 μL, 2 eq) and DMAP (2.93 mg, 24.0 μmol, 0.1 eq) at 0° C. The mixture was stirred at 20° C. for 0.5 hour. LC-MS showed 4-chloro-6-fluoro-3-[7-(4-fluorophenyl)-4, 7-diazaspiro [2.5]octan-4-yl]-1H-indazole was consumed completely and desired mass was detected. The reaction mixture was added to 20 mL of water, extracted with 30 mL of EtOAc. The combined organic layers were washed with 20 mL of brine and dried over Na₂SO₄, filtered and concentrated under reduced pressure to give a residue. The residue was purified by prep-HPLC (neutral condition; column: Phenomenex C18 75*30 mm*3 um; mobile phase: [water(NH₄HCO₃)-ACN]; B %: 50%-80%, 8 min) to give desired 4-chloro-6-fluoro-3-[7-(4-fluorophenyl)-4, 7-diazaspiro [2.5]octan-4-yl]-1-(1-isopropylsulfonylpyrrol-3-yl)sulfonyl-indazole (40.3 mg, 65.0 μmol, 27.1% yield, 98.43% purity) as a plate yellow solid. ¹HNMR (400 MHz, DMSO-d₆) δ 8.08-8.02 (m, 1H), 7.82 (dd, J=1.8, 8.8 Hz, 1H), 7.55 (dd, J=1.8, 9.1 Hz, 1H), 7.34 (t, J=2.7 Hz, 1H), 7.11-7.03 (m, 2H), 7.01-6.94 (m, 2H), 6.61-6.53 (m, 1H), 4.00-3.86 (m, 1H), 3.63 (br s, 2H), 3.36 (s, 2H), 3.09 (br s, 2H), 1.07 (d, J=6.7 Hz, 6H), 0.85 (br s, 2H), 0.52 (br s, 2H). HPLC: 98.43% (220 nm), 98.68% (215 nm), 99.26% (254 nm). MS (ESI): mass calcd. For C₂₆H₂₆ClF₂N₅O₄S₂ 609.11, m/z found 610.1 [M+H]⁺.

### Compound 176: 4-chloro-6-fluoro-1-(1-isopropylsulfonylpyrrol-3-yl) sulfonyl-3-[7-(4-pyridyl)-4, 7-diazaspiro [2.5]octan-4-yl]indazole

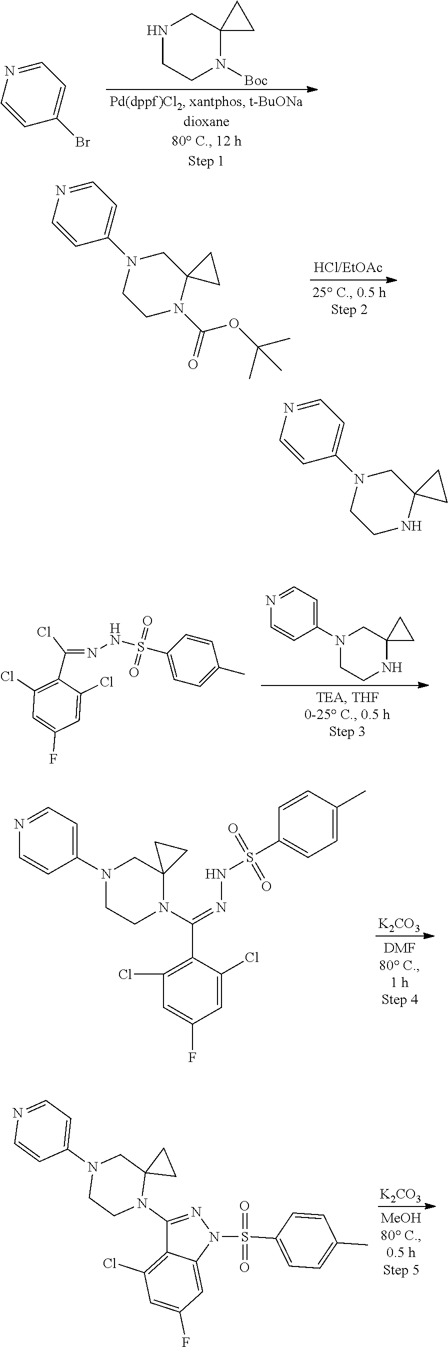

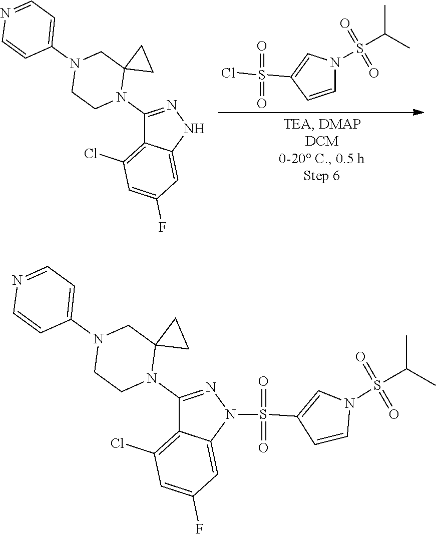 [SMILES: Fc1cc(Cl)c2c(N3CCN(c4ccncc4)CC34CC4)n[nH]c2c1 ; CC(C)S(=O)(=O)n1ccc(S(=O)(=O)Cl)c1 ; CC(C)S(=O)(=O)n1ccc(S(=O)(=O)n2nc(N3CCN(c4ccncc4)CC34CC4)c3c(Cl)cc(F)cc32)c1]

Step 1: A mixture of 4-bromopyridine (500 mg, 3.16 mmol, 1 eq), tert-butyl 4, 7-diazaspiro [2.5]octane-4-carboxylate (672 mg, 3.16 mmol, 1 eq), Xantphos (366 mg, 633 μmol, 0.2 eq), t-BuONa (912 mg, 9.49 mmol, 3 eq) and Pd(dppf)Cl₂ (232 mg, 316 μmol, 0.1 eq) in dioxane (10 mL) was degassed and purged with N₂ for 3 times, and then the mixture was stirred at 80° C. for 12 hours under N₂ atmosphere. LC-MS showed 4-bromopyridine was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/petroleum ether gradient @80 mL/min) to give desired tert-butyl 7-(4-pyridyl)-4, 7-diazaspiro [2.5]octane-4-carboxylate (690 mg, crude) as a yellow solid. MS (ESI): mass calcd. For C₁₆H₂₃N₃O₂ 289.18, m/z found 290.1 [M+H]⁺.

Step 2: A mixture of tert-butyl 7-(4-pyridyl)-4, 7-diazaspiro [2.5]octane-4-carboxylate (690 mg, 2.38 mmol, 1 eq) in HCl/EtOAc (5 mL) was stirred at 25° C. for 0.5 hour. LC-MS showed tert-butyl 7-(4-pyridyl)-4, 7-diazaspiro [2.5]octane-4-carboxylate was consumed completely and desired mass was detected. The reaction mixture was concentrated to give desired 7-(4-pyridyl)-4, 7-diazaspiro [2.5]octane (650 mg, crude, HCl) as a yellow solid. MS (ESI): mass calcd. For C₁₁H₁₅N₃ 189.13, m/z found 190.2 [M+H]⁺.

Step 3: To a solution of 7-(4-pyridyl)-4, 7-diazaspiro [2.5]octane (390 mg, 1.73 mmol, 1 eq, HCl) in THF (10 mL) was added dropwise TEA (1.75 g, 17.3 mmol, 2.40 mL, 10 eq) at 25° C. After addition, the mixture was stirred at this temperature for 10 mins, and then (1Z)-2, 6-dichloro-4-fluoro-N-(p-tolylsulfonyl)benzohydrazonoyl chloride (750 mg, 1.90 mmol, 1.1 eq) in THF (2 mL) was added dropwise at 0° C. The resulting mixture was stirred at 25° C. for 20 mins. LC-MS showed 7-(4-pyridyl)-4, 7-diazaspiro [2.5]octane was consumed completely and one main peak with desired mass was detected. Then it was separated between 20 mL of water and 40 mL of ethyl acetate. The organic phase was separated, washed with 30 mL of brine, dried over Na₂SO₄, filtered and concentrated under reduced pressure to give desired N—[(Z)-[(2, 6-dichloro-4-fluoro-phenyl)-[7-(4-pyridyl)-4, 7-diazaspiro [2.5]octan-4-yl]methylene]amino]-4-methyl-benzenesulfonamide (1.2 g, crude) as a yellow solid. MS (ESI): mass calcd. For C₂₅H₂₄Cl₂FN₅O₂S 547.10, m/z found 548.2 [M+H]⁺.

Step 4: To a solution of N—[(Z)-[(2, 6-dichloro-4-fluoro-phenyl)-[7-(4-pyridyl)-4, 7-diazaspiro [2.5]octan-4-yl]methylene]amino]-4-methyl-benzenesulfonamide (1.2 g, 2.19 mmol, 1 eq) in DMF (5 mL) was added K₂CO₃ (1.51 g, 10.9 mmol, 5 eq). The mixture was stirred at 80° C. for 1 hour. LC-MS showed N—[(Z)-[(2, 6-dichloro-4-fluoro-phenyl)-[7-(4-pyridyl)-4, 7-diazaspiro [2.5]octan-4-yl]methylene]amino]-4-methyl-benzenesulfonamide was consumed completely and desired mass was detected. The reaction mixture was added to water (20 mL), the aqueous phase was extracted with EtOAc (20 mL*3). The organic layer was dried over Na₂SO₄, filtered and the filtrate was concentrated to give desired 4-chloro-6-fluoro-1-(p-tolylsulfonyl)-3-[7-(4-pyridyl)-4, 7-diazaspiro [2.5]octan-4-yl]indazole (1.1 g, crude) as a black oil. MS (ESI): mass calcd. For C₂₅H₂₃ClFN₅O₂S 511.12, m/z found 512.2 [M+H]⁺.

Step 5: To a solution of 4-chloro-6-fluoro-1-(p-tolylsulfonyl)-3-[7-(4-pyridyl)-4, 7-diazaspiro [2.5]octan-4-yl]indazole (1.1 g, 2.15 mmol, 1 eq) in MeOH (5 mL) was added K₂CO₃ (1.19 g, 8.59 mmol, 4 eq). The mixture was stirred at 80° C. for 0.5 hour. LC-MS showed 4-chloro-6-fluoro-1-(p-tolylsulfonyl)-3-[7-(4-pyridyl)-4, 7-diazaspiro [2.5]octan-4-yl]indazole was consumed completely and desired mass was detected. The reaction mixture was added to water (20 mL), the aqueous phase was extracted with EtOAc (20 mL*3). The organic layer was dried over Na₂SO₄, filtered and the filtrate was concentrated to give a residue. The residue was purified by prep-TLC (SiO₂, petroleum ether/Ethyl acetate=1/1) to give desired 4-chloro-6-fluoro-3-[7-(4-pyridyl)-4, 7-diazaspiro [2.5]octan-4-yl]-1H-indazole (200 mg, crude) as a white solid. MS (ESI): mass calcd. For C₁₈H₁₇ClFN₅ 357.12, m/z found 358.2 [M+H]⁺.

Step 6: To a solution of 4-chloro-6-fluoro-3-[7-(4-pyridyl)-4, 7-diazaspiro [2.5]octan-4-yl]-1H-indazole (80 mg, 224 μmol, 1 eq) and 1-isopropylsulfonylpyrrole-3-sulfonyl chloride (122 mg, 447 μmol, 2 eq) in DCM (2 mL) was added TEA (45.3 mg, 447 μmol, 62.2 μL, 2 eq) and DMAP (2.73 mg, 22.4 μmol, 0.1 eq) at 0° C. The mixture was stirred at 20° C. for 0.5 hour. LC-MS showed 4-chloro-6-fluoro-3-[7-(4-pyridyl)-4, 7-diazaspiro [2.5]octan-4-yl]-1H-indazole was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by prep-HPLC (neutral condition; column: Phenomenex Luna C18 75*30 mm*3 um; mobile phase: [water(NH₄HCO₃)-ACN]; B %: 20%-60%, 8 min) to give desired 4-chloro-6-fluoro-1-(1-isopropylsulfonylpyrrol-3-yl) sulfonyl-3-[7-(4-pyridyl)-4, 7-diazaspiro [2.5]octan-4-yl]indazole (6.1 mg, 9.95 μmol, 4.45% yield, 96.77% purity) as a white solid. ¹H NMR (400 MHz, DMSO-d₆) δ 8.25-8.22 (m, 2H), 8.12 (t, J=1.9 Hz, 1H), 7.87 (dd, J=2.1, 8.9 Hz, 1H), 7.61 (dd, J=2.1, 9.1 Hz, 1H), 7.39 (dd, J=2.4, 3.3 Hz, 1H), 6.93-6.86 (m, 2H), 6.62 (dd, J=1.6, 3.4 Hz, 1H), 4.03-3.95 (m, 1H), 3.67-3.65 (m, 2H), 3.62 (br s, 2H), 3.38 (br s, 2H), 1.12 (d, J=6.8 Hz, 6H), 0.89 (s, 2H), 0.56 (br s, 2H). HPLC: 96.77% (220 nm), 96.52% (215 nm), 98.74% (254 nm). MS (ESI): mass calcd. For C₂₅H₂₆ClFN₆O₄S₂ 592.11, m/z found 593.1 [M+H]⁺.

### Compound 177: 4-chloro-6-fluoro-3-[(2S)-4-(5-fluoropyrimidin-2-yl)-2-methyl-piperazin-1-yl]-1-(1-isopropylsulfonylpyrrol-3-yl) sulfonyl-indazole

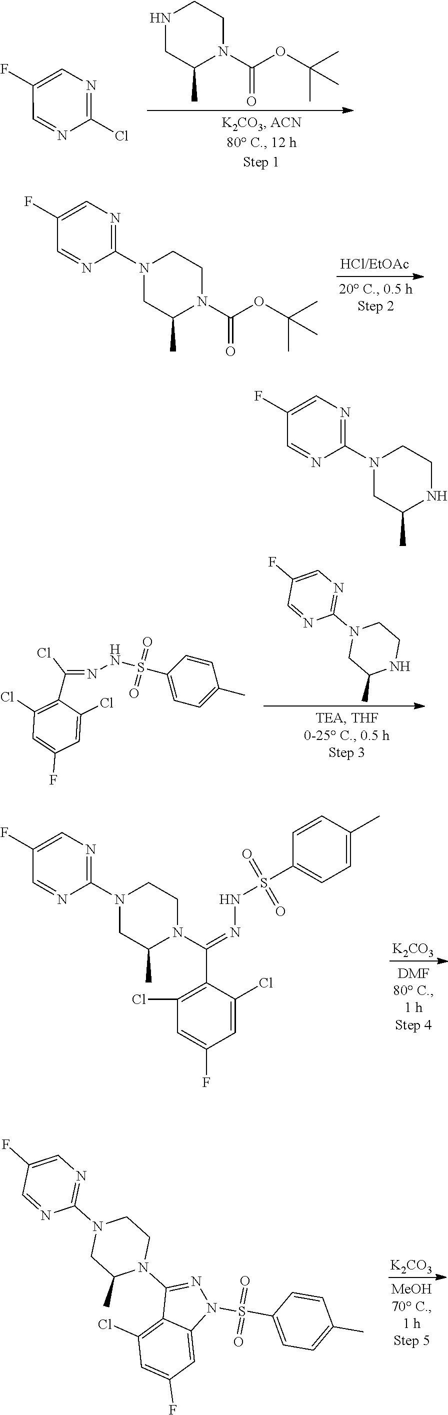

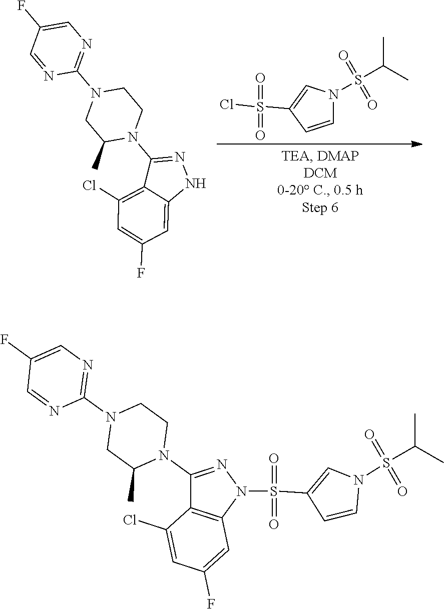 [SMILES: C[C@H]1CN(c2ncc(F)cn2)CCN1c1n[nH]c2cc(F)cc(Cl)c12 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cn4)C[C@@H]3C)c3c(Cl)cc(F)cc32)c1]

Step 1: To a solution of 2-chloro-5-fluoro-pyrimidine (500 mg, 3.77 mmol, 467 μL, 1 eq) and tert-butyl (2S)-2-methylpiperazine-1-carboxylate (831 mg, 4.15 mmol, 1.1 eq) in ACN (5 mL) was added K₂CO₃ (1.56 g, 11.3 mmol, 3 eq). The mixture was stirred at 80° C. for 12 hours. LC-MS showed 2-chloro-5-fluoro-pyrimidine was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by column chromatography (SiO₂, petroleum ether/Ethyl acetate=1/0 to 1/1) to give desired tert-butyl (2S)-4-(5-fluoropyrimidin-2-yl)-2-methyl-piperazine-1-carboxylate (800 mg, crude) as a colourless oil. MS (ESI): mass calcd. For C₁₄H₂₁FN₄O₂ 296.16, m/z found 241.1 [M+H]⁺.

Step 2: A mixture of tert-butyl (2S)-4-(5-fluoropyrimidin-2-yl)-2-methyl-piperazine-1-carboxylate (800 mg, 2.70 mmol, 1 eq) in HCl/EtOAc (4 mL) was stirred at 20° C. for 0.5 hour. LC-MS showed tert-butyl (2S)-4-(5-fluoropyrimidin-2-yl)-2-methyl-piperazine-1-carboxylate was consumed completely and desired mass was detected. The reaction mixture was concentrated to give desired 5-fluoro-2-[(3S)-3-methylpiperazin-1-yl]pyrimidine (700 mg, crude) as a yellow solid. MS (ESI): mass calcd. For C₉H₁₃FN₄ 196.11, m/z found 197.1 [M+H]⁺.

Step 3: To a solution of 5-fluoro-2-[(3S)-3-methylpiperazin-1-yl]pyrimidine (200 mg, 1.02 mmol, 1 eq) in THF (5 mL) was added dropwise TEA (1.03 g, 10.2 mmol, 1.42 mL, 10 eq) at 25° C. After addition, the mixture was stirred at this temperature for 10 mins, and then (1Z)-2, 6-dichloro-4-fluoro-N-(p-tolylsulfonyl)benzohydrazonoyl chloride (444 mg, 1.12 mmol, 1.1 eq) in THF (5 mL) was added dropwise at 0° C. The resulting mixture was stirred at 25° C. for 20 mins. LC-MS showed 5-fluoro-2-[(3S)-3-methylpiperazin-1-yl]pyrimidine was consumed completely and one main peak with desired mass was detected. Then it was separated between 20 mL of water and 40 mL of ethyl acetate. The organic phase was separated, washed with brine (30 mL), dried over Na₂SO₄, filtered and concentrated under reduced pressure to give desired N—[(Z)-[(2, 6-dichloro-4-fluoro-phenyl)-[(2S)-4-(5-fluoropyrimidin-2-yl)-2-methyl-piperazin-1-yl]methylene]amino]-4-methyl-benzenesulfonamide (600 mg, crude) as a yellow solid. MS (ESI): mass calcd. For C₂₃H₂₂Cl₂F₂N₆O₂S 554.09, m/z found 555.2 [M+H]⁺.

Step 4: To a solution of N—[(Z)-[(2, 6-dichloro-4-fluoro-phenyl)-[(2S)-4-(5-fluoropyrimidin-2-yl)-2-methyl-piperazin-1-yl]methylene]amino]-4-methyl-benzenesulfonamide (600 mg, 1.08 mmol, 1 eq) in DMF (10 mL) was added K₂CO₃ (597 mg, 4.32 mmol, 4 eq). The mixture was stirred at 80° C. for 1 hour. LC-MS showed N—[(Z)-[(2, 6-dichloro-4-fluoro-phenyl)-[(2S)-4-(5-fluoropyrimidin-2-yl)-2-methyl-piperazin-1-yl]methylene]amino]-4-methyl-benzenesulfonamide was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give desired 4-chloro-6-fluoro-3-[(2S)-4-(5-fluoropyrimidin-2-yl)-2-methyl-piperazin-1-yl]-1-(p-tolylsulfonyl) indazole (560 mg, crude) as a black oil. MS (ESI): mass calcd. For C₂₃H₂₁ClF₂N₆O₂S 518.11, m/z found 519.2 [M+H]⁺.

Step 5: To a solution of 4-chloro-6-fluoro-3-[(2S)-4-(5-fluoropyrimidin-2-yl)-2-methyl-piperazin-1-yl]-1-(p-tolylsulfonyl) indazole (560 mg, 1.08 mmol, 1 eq) in MeOH (20 mL) was added K₂CO₃ (542 mg, 3.92 mmol, 5 eq). The mixture was stirred at 70° C. for 1 hour. LC-MS showed 4-chloro-6-fluoro-3-[(2S)-4-(5-fluoropyrimidin-2-yl)-2-methyl-piperazin-1-yl]-1-(p-tolylsulfonyl) indazole was consumed completely and desired compound was detected. The reaction mixture was added to water (20 mL), extracted with EtOAc (10 mL*3). The combined organic layers were washed with brine (20 Ml) and dried over Na₂SO₄, filtered and concentrated under reduced pressure to give a residue. The residue was purified by column chromatography (SiO₂, petroleum ether/Ethyl acetate=1/0 to 1/1) to give desired 4-chloro-6-fluoro-3-[(2S)-4-(5-fluoropyrimidin-2-yl)-2-methyl-piperazin-1-yl]-1H-indazole (150 mg, crude) as a yellow oil. MS (ESI): mass calcd. For C₁₆H₁₅ClF₂N₆ 364.10, m/z found 365.1 [M+H]⁺.

Step 6: To a solution of 1-isopropylsulfonylpyrrole-3-sulfonyl chloride (74.5 mg, 274 mol, 2 eq) and 4-chloro-6-fluoro-3-[(2S)-4-(5-fluoropyrimidin-2-yl)-2-methyl-piperazin-1-yl]-1H-indazole (50 mg, 137 μmol, 1 eq) in DCM (2 mL) was added TEA (27.7 mg, 274 μmol, 38.2 μL, 2 eq) and DMAP (1.67 mg, 13.7 μmol, 0.1 eq) at 0° C. The mixture was stirred at 20° C. for 0.5 hour. LC-MS showed 4-chloro-6-fluoro-3-[(2S)-4-(5-fluoropyrimidin-2-yl)-2-methyl-piperazin-1-yl]-1H-indazole was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by prep-HPLC (neutral condition; column: Waters Xbridge BEH C18 100*30 mm*10 um; mobile phase: [water(NH₄HCO₃)-ACN]; B %: 65%-95%, 7 min) to give desired 4-chloro-6-fluoro-3-[(2S)-4-(5-fluoropyrimidin-2-yl)-2-methyl-piperazin-1-yl]-1-(1-isopropylsulfonylpyrrol-3-yl)sulfonyl-indazole (19.9 mg, 32.5 μmol, 23.7% yield, 97.85% purity) as a yellow solid. ¹H NMR (400 MHz, DMSO-d₆) δ 8.49 (s, 2H), 8.10 (d, J=1.5 Hz, 1H), 7.85 (dd, J=2.0, 8.9 Hz, 1H), 7.59 (dd, J=2.1, 9.1 Hz, 1H), 7.41-7.32 (m, 1H), 6.61 (dd, J=1.5, 3.1 Hz, 1H), 4.13 (br d, J=13.6 Hz, 1H), 3.96-3.88 (m, 2H), 3.87-3.82 (m, 1H), 3.75 (br d, J=11.8 Hz, 1H), 3.65-3.58 (m, 1H), 3.51-3.44 (m, 1H), 3.08 (br d, J=12.8 Hz, 1H), 1.11-1.06 (m, 6H), 0.97 (br d, J=6.3 Hz, 3H). HPLC: 97.85% (220 nm), 98.53% (215 nm), 97.53% (254 nm). MS (ESI): mass calcd. For C₂₃H₂₄ClF₂N₇O₄S₂ 599.10, m/z found 600.1 [M+H]⁺.

### Compound 178: 4-chloro-6-fluoro-3-[(2R)-4-(5-fluoropyrimidin-2-yl)-2-methyl-piperazin-1-yl]-1-(1-isopropylsulfonylpyrrol-3-yl) sulfonyl-indazole

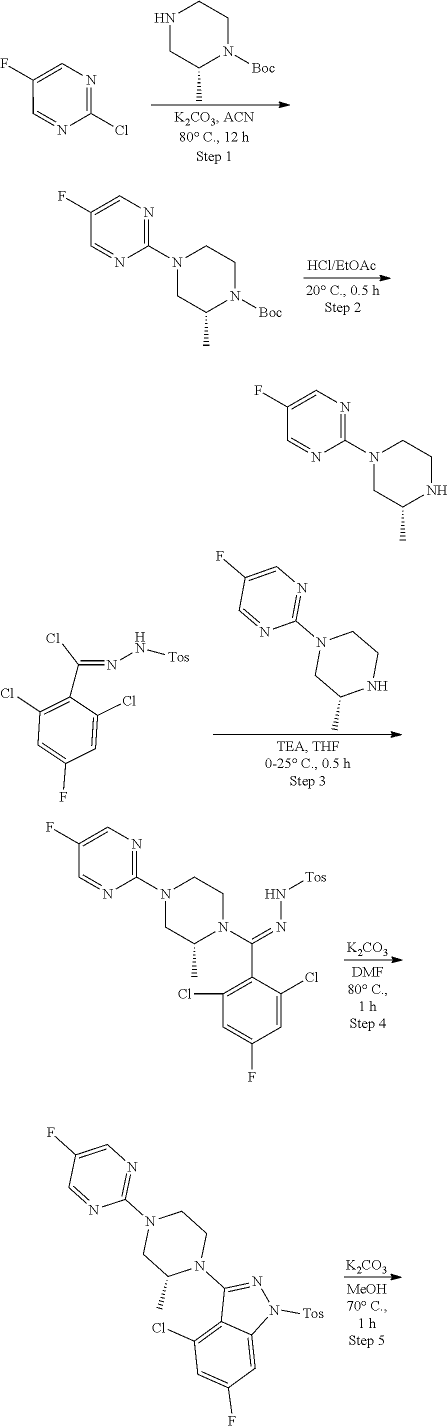

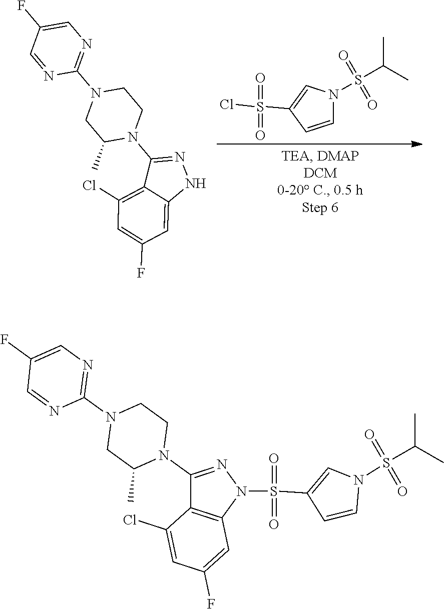 [SMILES: C[C@@H]1CN(c2ncc(F)cn2)CCN1c1n[nH]c2cc(F)cc(Cl)c12 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cn4)C[C@H]3C)c3c(Cl)cc(F)cc32)c1]

Step 1: To a solution of 2-chloro-5-fluoro-pyrimidine (500 mg, 3.77 mmol, 467 μL, 1 eq) and tert-butyl (2R)-2-methylpiperazine-1-carboxylate (831 mg, 4.15 mmol, 1.1 eq) in ACN (5 mL) was added K₂CO₃ (1.56 g, 11.3 mmol, 3 eq). The mixture was stirred at 80° C. for 12 hours. LC-MS showed 2-chloro-5-fluoro-pyrimidine was consumed completely and desired mass was detected. The reaction mixture was added to 20 mL of water, extracted with 30 mL of EtOAc. The combined organic layers were washed with 20 mL of brine and dried over Na₂SO₄, filtered and concentrated under reduced pressure to give a residue. The residue was purified by column chromatography (SiO₂, petroleum ether/EtOAc=1/0 to 1/1) to give desired tert-butyl (2R)-4-(5-fluoropyrimidin-2-yl)-2-methyl-piperazine-1-carboxylate (900 mg, crude) as a colourless oil. MS (ESI): mass calcd. For C₁₄H₂₁FN₄O₂ 296.16, m/z found 241.1 [M+H−56]⁺.

Step 2: A mixture of tert-butyl (2R)-4-(5-fluoropyrimidin-2-yl)-2-methyl-piperazine-1-carboxylate (900 mg, 3.04 mmol, 1 eq) in HCl/EtOAc (4 mL) was stirred at 20° C. for 0.5 hour. LC-MS showed tert-butyl (2R)-4-(5-fluoropyrimidin-2-yl)-2-methyl-piperazine-1-carboxylate was consumed completely and desired mass was detected. The reaction mixture was concentrated to give desired 5-fluoro-2-[(3R)-3-methylpiperazin-1-yl]pyrimidine (650 mg, crude, HCl) as a yellow solid. MS (ESI): mass calcd. For C₉H₁₃FN₄ 196.11, m/z found 197.1 [M+H]⁺.

Step 3: To a solution of 5-fluoro-2-[(3R)-3-methylpiperazin-1-yl]pyrimidine (200 mg, 860 μmol, 1 eq, HCl) in THF (3 mL) was added dropwise TEA (870 mg, 8.60 mmol, 1.20 mL, 10 eq) at 25° C. After addition, the mixture was stirred 25° C. for 10 mins, and then (1Z)-2, 6-dichloro-4-fluoro-N-(p-tolylsulfonyl) benzohydrazonoyl chloride (374 mg, 945 μmol, 1.1 eq) in THF (3 mL) was added dropwise at 0° C. The resulting mixture was stirred at 25° C. for 20 mins. LC-MS showed 5-fluoro-2-[(3R)-3-methylpiperazin-1-yl]pyrimidine was consumed completely and one main peak with desired mass was detected. Then it was separated between 20 mL of water and 40 mL of EtOAc. The organic phase was separated, washed with 30 mL of brine, dried over Na₂SO₄, filtered and concentrated under reduced pressure to give desired N—[(Z)-[(2, 6-dichloro-4-fluoro-phenyl)-[(2R)-4-(5-fluoropyrimidin-2-yl)-2-methyl-piperazin-1-yl]methylene]amino]-4-methyl-benzenesulfonamide (470 mg, crude) as a yellow solid. MS (ESI): mass calcd. For C₂₃H₂₂Cl₂F₂N₆O₂S 554.09, m/z found 555.0 [M+H]⁺.

Step 4: To a solution of N—[(Z)-[(2, 6-dichloro-4-fluoro-phenyl)-[(2R)-4-(5-fluoropyrimidin-2-yl)-2-methyl-piperazin-1-yl]methylene]amino]-4-methyl-benzenesulfonamide (470 mg, 846 μmol, 1 eq) in DMF (10 mL) was added K₂CO₃ (468 mg, 3.38 mmol, 4 eq). The mixture was stirred at 80° C. for 1 hour. LC-MS showed N—[(Z)-[(2, 6-dichloro-4-fluoro-phenyl)-[(2R)-4-(5-fluoropyrimidin-2-yl)-2-methyl-piperazin-1-yl]methylene]amino]-4-methyl-benzenesulfonamide was consumed completely and desired mass was detected. The reaction mixture was added to 20 mL of water, extracted with 30 mL of EtOAc. The combined organic layers were washed with 20 mL of brine and dried over Na₂SO₄, filtered and concentrated under reduced pressure to give desired 4-chloro-6-fluoro-3-[(2R)-4-(5-fluoropyrimidin-2-yl)-2-methyl-piperazin-1-yl]-1-(p-tolylsulfonyl) indazole (440 mg, crude) as a black oil. MS (ESI): mass calcd. For C₂₃H₂₁ClF₂N₆O₂S 518.11, m/z found 519.1 [M+H]⁺.

Step 5: To a solution of 4-chloro-6-fluoro-3-[(2R)-4-(5-fluoropyrimidin-2-yl)-2-methyl-piperazin-1-yl]-1-(p-tolylsulfonyl) indazole (440 mg, 848 μmol, 1 eq) in MeOH (20 mL) was added K₂CO₃ (586 mg, 4.24 mmol, 5 eq). The mixture was stirred at 70° C. for 1 hour. LC-MS showed 4-chloro-6-fluoro-3-[(2R)-4-(5-fluoropyrimidin-2-yl)-2-methyl-piperazin-1-yl]-1-(p-tolylsulfonyl) indazole was consumed completely and desired compound was detected. The reaction mixture was added to 20 mL of water, extracted with 30 mL of EtOAc. The combined organic layers were washed with 20 mL of brine and dried over Na₂SO₄, filtered and concentrated under reduced pressure to give a residue. The residue was purified by column chromatography (SiO₂, petroleum ether/EtOAc=1/0 to 1/1) to give desired 4-chloro-6-fluoro-3-[(2R)-4-(5-fluoropyrimidin-2-yl)-2-methyl-piperazin-1-yl]-1H-indazole (150 mg, crude) as a yellow oil. MS (ESI): mass calcd. For C₁₆H₁₅ClF₂N₆ 364.10, m/z found 365.1 [M+H]⁺.

Step 6: To a solution of 1-isopropylsulfonylpyrrole-3-sulfonyl chloride (74.5 mg, 274 mol, 2 eq) and 4-chloro-6-fluoro-3-[(2R)-4-(5-fluoropyrimidin-2-yl)-2-methyl-piperazin-1-yl]-1H-indazole (50 mg, 137 μmol, 1 eq) in DCM (2 mL) was added TEA (27.7 mg, 274 μmol, 38.2 μL, 2 eq) and DMAP (1.67 mg, 13.7 μmol, 0.1 eq) at 0° C. The mixture was stirred at 20° C. for 0.5 hour. LC-MS showed 4-chloro-6-fluoro-3-[(2R)-4-(5-fluoropyrimidin-2-yl)-2-methyl-piperazin-1-yl]-1H-indazole was consumed completely and desired mass was detected. The reaction mixture was added to 20 mL of water, extracted with 30 mL of EtOAc. The combined organic layers were washed with 20 mL of brine and dried over Na₂SO₄, filtered and concentrated under reduced pressure to give a residue. The residue was purified by prep-HPLC (neutral condition; column: Waters Xbridge BEH C18 100*30 mm*10 um; mobile phase: [water(NH₄HCO₃)-ACN]; B %: 60%-90%, 7 min) to give desired 4-chloro-6-fluoro-3-[(2R)-4-(5-fluoropyrimidin-2-yl)-2-methyl-piperazin-1-yl]-1-(1-isopropylsulfonylpyrrol-3-yl)sulfonyl-indazole (20.2 mg, 33.3 μmol, 24.3% yield, 99.02% purity) as a white solid. ¹H NMR (400 MHz, DMSO-d₆) δ 8.48 (s, 2H), 8.09 (s, 1H), 7.84 (dd, J=1.6, 8.8 Hz, 1H), 7.57 (dd, J=1.6, 9.0 Hz, 1H), 7.35 (t, J=2.6 Hz, 1H), 6.61 (br d, J=1.6 Hz, 1H), 4.17-4.07 (m, 1H), 4.00-3.81 (m, 3H), 3.76 (br d, J=12.4 Hz, 1H), 3.61 (br t, J=9.5 Hz, 1H), 3.47 (br t, J=9.3 Hz, 1H), 3.13-3.02 (m, 1H), 1.08 (dd, J=3.1, 6.6 Hz, 6H), 0.96 (d, J=6.1 Hz, 3H). HPLC: 99.02% (220 nm), 98.96% (215 nm), 99.10% (254 nm). MS (ESI): mass calcd. For C₂₃H₂₄ClF₂N₇O₄S₂ 599.10, m/z found 600.1 [M+H]⁺.

### Compound 179: 4-chloro-6-fluoro-3-(8-(5-fluoropyrimidin-2-yl)-5, 8-diazaspiro [3.5]nonan-5-yl)-1-((1-(isopropylsulfonyl)-1H-pyrrol-3-yl) sulfonyl)-1H-indazole

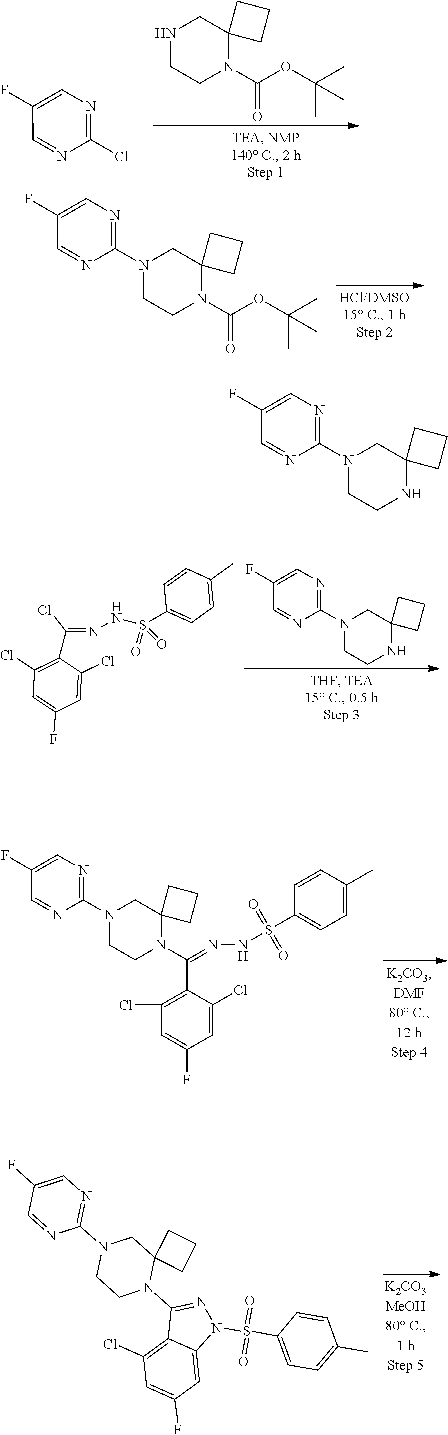

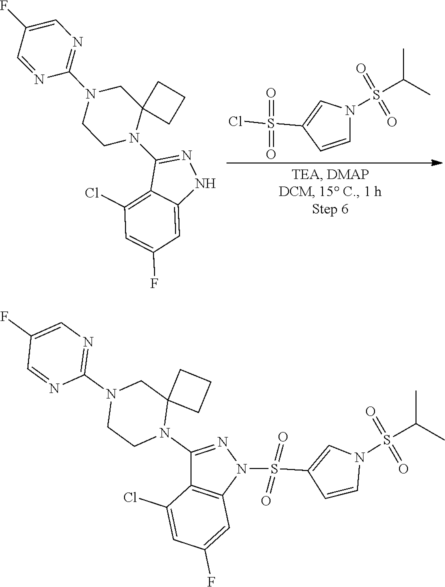 [SMILES: Fc1cnc(N2CCN(c3n[nH]c4cc(F)cc(Cl)c34)C3(CCC3)C2)nc1 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cn4)CC34CCC4)c3c(Cl)cc(F)cc32)c1]

Step 1: To a solution of 2-chloro-5-fluoro-pyrimidine (293 mg, 2.21 mmol, 274 μL, 1 eq) and tert-butyl 5, 8-diazaspiro [3.5]nonane-5-carboxylate (500 mg, 2.21 mmol, 1 eq) in NMP (10 mL) was added TEA (671 mg, 6.63 mmol, 923 μL, 3 eq). The mixture was stirred at 140° C. for 2 hours. LC-MS showed 2-chloro-5-fluoro-pyrimidine was consumed completely and desired mass was detected. The reaction mixture was added to water (20 mL), extracted with EtOAc (10 mL*3). The combined organic layers were washed with brine (20 mL) and dried over Na₂SO₄, filtered and concentrated under reduced pressure to give a residue. The residue was purified by column prep-TLC (SiO₂, petroleum ether/Ethyl acetate=4/1) to give desired tert-butyl 8-(5-fluoropyrimidin-2-yl)-5, 8-diazaspiro [3.5]nonane-5-carboxylate (600 mg, 1.86 mmol, 84.2% yield) as a white solid. MS (ESI): mass calcd. For C₁₆H₂₃FN₄O₂ 322.18, m/z found 323.3 [M+H]⁺.

Step 2: To a solution of tert-butyl 8-(5-fluoropyrimidin-2-yl)-5, 8-diazaspiro [3.5]nonane-5-carboxylate (600 mg, 1.86 mmol, 1 eq) in HCl/DMSO (10 mL). The mixture was stirred at 15° C. for 1 hour. TLC (SiO₂, petroleum ether/Ethyl acetate=4/1) showed tert-butyl 8-(5-fluoropyrimidin-2-yl)-5, 8-diazaspiro [3.5]nonane-5-carboxylate was consumed completely and desired mass was detected. The reaction mixture was concentrated to give desired 8-(5-fluoropyrimidin-2-yl)-5, 8-diazaspiro [3.5]nonane (480 mg, crude, HCl) as a yellow solid.

Step 3: To a solution of 8-(5-fluoropyrimidin-2-yl)-5,8-diazaspiro[3.5]nonane (200 mg, 900 μmol, 1 eq) in THF (5 mL) was added TEA (228 mg, 2.25 mmol, 313 μL, 2.5 eq) and (1Z)-2,6-dichloro-4-fluoro-N-(p-tolylsulfonyl)benzohydrazonoyl chloride (427 mg, 1.08 mmol, 1.2 eq). The mixture was stirred at 15° C. for 0.5 hour. LC-MS showed 8-(5-fluoropyrimidin-2-yl)-5, 8-diazaspiro [3.5]nonane was consumed completely and desired mass was detected. The crude was added H₂O (10 mL), and extracted with EtOAc (45 mL). The combined organic layers were washed with brine (10 mL), dried over Na₂SO₄, filtered and concentrated under reduced pressure to give desired (E)-N′-((2,6-dichloro-4-fluorophenyl)(8-(5-fluoropyrimidin-2-yl)-5,8-diazaspiro[3.5]nonan-5-yl)methylene)-4-methylbenzenesulfonohydrazide (500 mg, crude) as a yellow solid. MS (ESI): mass calcd. For C₂₅H₂₄Cl₂F₂N₆O₂S 580.10, m/z found 581.2 [M+H]⁺.

Step 4: To a solution of N—[(Z)-[(2, 6-dichloro-4-fluoro-phenyl)-[8-(5-fluoropyrimidin-2-yl)-5, 8-diazaspiro [3.5]nonan-5-yl]methylene]amino]-4-methyl-benzenesulfonamide (500 mg, 860 μmol, 1 eq) in DMF (10 mL) was added K₂CO₃ (1.19 g, 8.60 mmol, 10 eq). The mixture was stirred at 80° C. for 12 hours. LC-MS showed N—[(Z)-[(2, 6-dichloro-4-fluoro-phenyl)-[8-(5-fluoropyrimidin-2-yl)-5, 8-diazaspiro [3.5]nonan-5-yl]methylene]amino]-4-methyl-benzenesulfonamide was consumed completely and desired mass was detected. The crude was added H₂O (10 mL), and extracted with EtOAc (45 mL). The combined organic layers were washed with brine (10 mL), dried over Na₂SO₄, filtered and concentrated under reduced pressure to give desired 4-chloro-6-fluoro-3-(8-(5-fluoropyrimidin-2-yl)-5, 8-diazaspiro [3.5]nonan-5-yl)-1-tosyl-1H-indazole (300 mg, crude) as a yellow oil. MS (ESI): mass calcd. For C₂₅H₂₃ClF₂N₆O₂S 544.13, m/z found 545.2 [M+H]⁺.

Step 5: To a solution of 4-chloro-6-fluoro-3-[8-(5-fluoropyrimidin-2-yl)-5, 8-diazaspiro [3.5]nonan-5-yl]-1-(p-tolylsulfonyl) indazole (300 mg, 550 μmol, 1 eq) in MeOH (5 mL) was added K₂CO₃ (380 mg, 2.75 mmol, 5 eq). The mixture was stirred at 80° C. for 1 hour. LC-MS showed 4-chloro-6-fluoro-3-[8-(5-fluoropyrimidin-2-yl)-5, 8-diazaspiro [3.5]nonan-5-yl]-1-(p-tolylsulfonyl) indazole was consumed completely and desired mass was detected. The reaction mixture was concentrated under reduced pressure to give a residue. The crude was added H₂O (20 mL), and extracted with EtOAc (45 mL). The combined organic layers were washed with brine (10 mL), dried over Na₂SO₄, filtered and concentrated under reduced pressure to give a residue. The residue was purified by column prep-TLC (SiO₂, petroleum ether/Ethyl acetate=2/1) to give desired 4-chloro-6-fluoro-3-(8-(5-fluoropyrimidin-2-yl)-5, 8-diazaspiro [3.5]nonan-5-yl)-1H-indazole (160 mg, 409 μmol, 74.4% yield) as a yellow oil. MS (ESI): mass calcd. For C₁₈H₁₇ClF₂N₆ 390.12, m/z found 391.1 [M+H]⁺.

Step 6: To a solution of 4-chloro-6-fluoro-3-[8-(5-fluoropyrimidin-2-yl)-5,8-diazaspiro[3.5]nonan-5-yl]-1H-indazole (80 mg, 205 μmol, 1 eq) and 1-isopropylsulfonylpyrrole-3-sulfonyl chloride (111 mg, 409 μmol, 2 eq) in DCM (2 mL) was added TEA (41.4 mg, 409 μmol, 57.0 μL, 2 eq) and DMAP (2.50 mg, 20.5 μmol, 0.1 eq). The mixture was stirred at 15° C. for 1 hour. LC-MS showed 4-chloro-6-fluoro-3-[8-(5-fluoropyrimidin-2-yl)-5, 8-diazaspiro [3.5]nonan-5-yl]-1H-indazole was consumed completely and desired mass was detected. The reaction mixture was concentrated under reduced pressure to give a residue. The residue was purified by prep-HPLC (neutral condition, column: Phenomenex Luna C18 75*30 mm*3 um; mobile phase: [water(FA)-ACN];B %: 65%-95%, 8 min) to give desired 4-chloro-6-fluoro-3-(8-(5-fluoropyrimidin-2-yl)-5, 8-diazaspiro [3.5]nonan-5-yl)-1-((1-(isopropylsulfonyl)-1H-pyrrol-3-yl) sulfonyl)-1H-indazole (10.3 mg, 16.3 μmol, 7.94% yield, 98.85% purity) as a white solid. ¹H NMR (400 MHz, DMSO-d₆) δ 8.48 (s, 2H), 8.09 (t, J=2.0 Hz, 1H), 7.84 (dd, J=2.0, 8.9 Hz, 1H), 7.55 (dd, J=2.1, 9.1 Hz, 1H), 7.37 (dd, J=2.4, 3.3 Hz, 1H), 6.57 (dd, J=1.6, 3.4 Hz, 1H), 4.06 (s, 2H), 3.99-3.90 (m, 1H), 3.69-3.54 (m, 2H), 3.32 (br s, 2H), 1.99-1.85 (m, 4H), 1.80-1.61 (m, 2H), 1.10 (d, J=6.8 Hz, 6H). HPLC: 98.85% (220 nm), 98.96% (215 nm), 99.13% (254 nm). MS (ESI): mass calcd. For C₂₅H₂₆ClF₂N₇O₄S₂ 625.11 m/z found 625.6 [M+H]⁺.

### Compound 180: 4-chloro-6-fluoro-3-[7-(5-fluoropyrimidin-2-yl)-4, 7-diazaspiro [2.5]octan-4-yl]-1-(2, 4, 5-trideuterio-1-isopropylsulfonyl-pyrrol-3-yl) sulfonyl-indazole

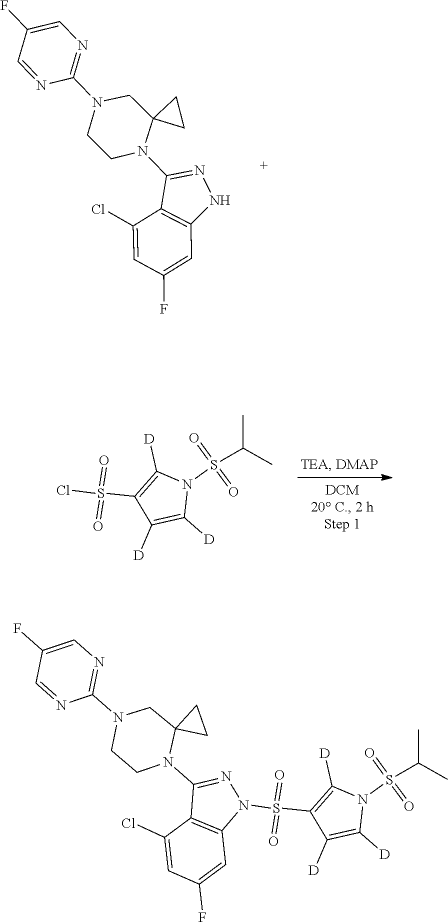 [SMILES: Fc1cnc(N2CCN(c3n[nH]c4cc(F)cc(Cl)c34)C3(CC3)C2)nc1 ; [2H]c1c(S(=O)(=O)Cl)c([2H])n(S(=O)(=O)C(C)C)c1[2H] ; [2H]c1c(S(=O)(=O)n2nc(N3CCN(c4ncc(F)cn4)CC34CC4)c3c(Cl)cc(F)cc32)c([2H])n(S(=O)(=O)C(C)C)c1[2H]]

Step 1: To the solution of 4-chloro-6-fluoro-3-[7-(5-fluoropyrimidin-2-yl)-4, 7-diazaspiro [2.5]octan-4-yl]-1H-indazole (50 mg, 133 μmol, 1 eq), TEA (40.3 mg, 398 μmol, 3 eq) and DMAP (1.62 mg, 13.3 μmol, 0.1 eq) in DCM (3 mL) was added 2,4,5-trideuterio-1-isopropylsulfonyl-pyrrole-3-sulfonyl chloride (36.7 mg, 133 μmol, 1 eq) at 20° C. and the solution was stirred at 20° C. for 2 hours. LCMS showed 4-chloro-6-fluoro-3-[7-(5-fluoropyrimidin-2-yl)-4, 7-diazaspiro [2.5]octan-4-yl]-1H-indazole remained and desired mass was detected. The reaction was concentrated to get a residue. The residue was purified by prep-HPLC(column: Phenomenex luna C18 (250*70 mm, 15 um);mobile phase: [water(FA)-ACN]; B %: 80%-100%, 20 min) to give desired 4-chloro-6-fluoro-3-[7-(5-fluoropyrimidin-2-yl)-4,7-diazaspiro[2.5]octan-4-yl]-1-(2,4,5-trideuterio-1-isopropylsulfonyl-pyrrol-3-yl)sulfonyl-indazole (44 mg, 70.0 μmol, 52.7% yield, 97.84% purity) as a white solid. ¹H NMR (400 MHz, DMSO-d₆) δ 8.46 (s, 2H), 7.83 (m, 1H), 7.54 (m, 1H), 3.92 (s, 2H), 3.60-3.77 (m, 2H), 3.54 (br d, 2H), 1.78 (s, 6H), 0.78 (br s, 2H), 0.47 (br s, 2H) HPLC: 97.84% (220 nm), 97.03% (215 nm), 99.51% (254 nm). MS (ESI): mass calcd. For C₂₄H₂₁D₃ClF₂N₇O₄S₂ 614.12 m/z found 615.0 [M+H]⁺.

### Compound 181: 4-chloro-1-[1-(1-chloro-1-methyl-ethyl)sulfonyl-2,4,5-trideuterio-pyrrol-3-yl]sulfonyl-6-fluoro-3-[7-(5-fluoropyrimidin-2-yl)-4,7-diazaspiro[2.5]octan-4-yl]indazole

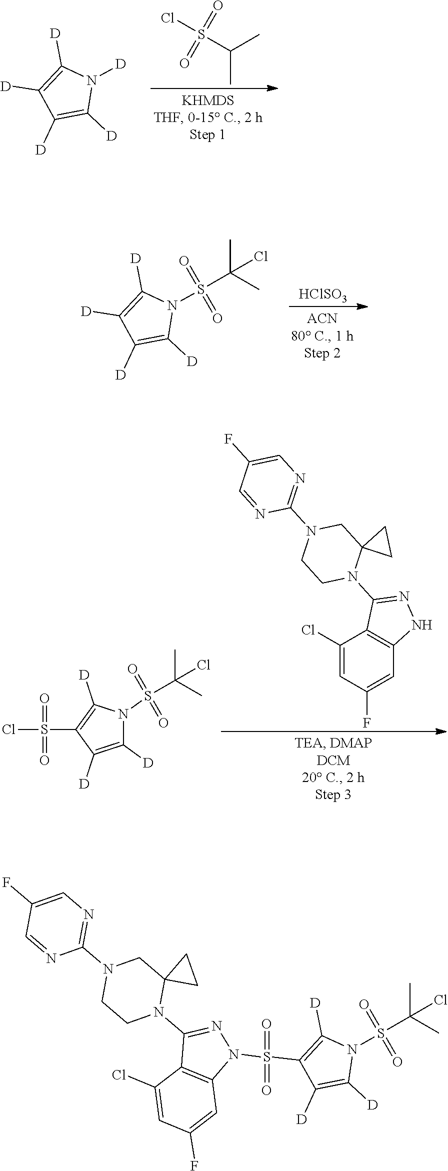

Step 1: To the solution of 1, 2, 3, 4, 5-pentadeuteriopyrrole (500 mg, 6.93 mmol, 1 eq) in THF (15 mL) was added KHMDS (1 M, 13.9 mL, 2 eq) at 0° C. under N₂ and the mixture was stirred at 0° C. for 0.5 hour. To the solution was added propane-2-sulfonyl chloride (1.48 g, 10.4 mmol, 1.16 mL, 1.5 eq) stirred at 0° C. and the solution was stirred at 15° C. for 1.5 hour. TLC showed 1, 2, 3, 4, 5-pentadeuteriopyrrole was consumed completely. The residue was purified by flash silica gel chromatography (ISCO®; 40 g SepaFlash® Silica Flash Column, Eluent of 0˜25% Ethylacetate/petroleum ether gradient @40 mL/min) to give desired 1-(1-chloro-1-methyl-ethyl) sulfonyl-2,3,4,5-tetradeuterio-pyrrole (420 mg, 1.98 mmol, 28.63% yield) as a colorless solid.

Step 2: To the solution of 1-(1-chloro-1-methyl-ethyl)sulfonyl-2,3,4,5-tetradeuterio-pyrrole (420 mg, 1.98 mmol, 1 eq) in ACN (10 mL) was added HSO₃Cl (1.16 g, 9.92 mmol, 5 eq) and the solution was stirred at 80° C. for 1 hour. TLC showed 1-(1-chloro-1-methyl-ethyl) sulfonyl-2, 3, 4, 5-tetradeuterio-pyrrole was consumed completely and a main new spot was detected. The reaction was poured into water (20 mL) and extracted with MTBE (2*10 mL). The combined organics were concentrated to get a residue. The residue was purified by flash silica gel chromatography (ISCO®; 20 g SepaFlash® Silica Flash Column, Eluent of 0˜20% Ethyl acetate/petroleum ether gradient @50 mL/min) to give desired 1-(1-chloro-1-methyl-ethyl)sulfonyl-2,4,5-trideuterio-pyrrole-3-sulfonyl chloride (300 mg, 970 μmol, 48.9% yield) as a white solid.

Step 3: To the solution of 4-chloro-6-fluoro-3-[7-(5-fluoropyrimidin-2-yl)-4,7-diazaspiro[2.5]octan-4-yl]-1H-indazole (50 mg, 133 μmol, 1 eq), TEA (40.3 mg, 398 μmol, 3 eq) and DMAP (1.62 mg, 13.3 μmol, 0.1 eq) in DCM (3 mL) was added 1-(1-chloro-1-methyl-ethyl)sulfonyl-2,4,5-trideuterio-pyrrole-3-sulfonyl chloride (41.0 mg, 133 μmol, 1 eq) at 20° C. and the solution was stirred at 20° C. for 2 hours. LCMS showed 4-chloro-6-fluoro-3-[7-(5-fluoropyrimidin-2-yl)-4, 7-diazaspiro [2.5]octan-4-yl]-1H-indazole remained and desired mass was detected. The reaction was concentrated to get a residue. The residue was purified by prep-HPLC (column: Phenomenex luna C18 (250*70 mm, 15 um);mobile phase: [water(FA)-ACN]; B %: 80%-100%, 20 min) to give desired 4-chloro-1-[1-(1-chloro-1-methyl-ethyl)sulfonyl-2,4,5-trideuterio-pyrrol-3-yl]sulfonyl-6-fluoro-3-[7-(5-fluoropyrimidin-2-yl)-4,7-diazaspiro[2.5]octan-4-yl]indazole (43.7 mg, 66.3 μmol, 50.0% yield, 98.61% purity) as a white solid. ¹H NMR (400 MHz, DMSO-d₆) δ 8.46 (s, 2H), 7.83 (m, 1H), 7.54 (m, 1H), 3.92 (s, 2H), 3.60-3.77 (m, 2H), 3.54 (br d, 2H), 1.78 (s, 6H), 0.78 (br s, 2H), 0.47 (br s, 2H) HPLC: 98.61% (220 nm), 97.84% (215 nm), 98.19% (254 nm). MS (ESI): mass calcd. For C₂₄H₂₀D₃Cl₂F₂N₇O₄S₂ 648.08 m/z found 649.2 [M+H]⁺.

### Compound 182: 4-chloro-3-(7-(2, 6-difluorophenyl)-4, 7-diazaspiro [2.5]octan-4-yl)-6-fluoro-1-((1-(isopropylsulfonyl)-1H-pyrrol-3-yl) sulfonyl)-1H-indazole

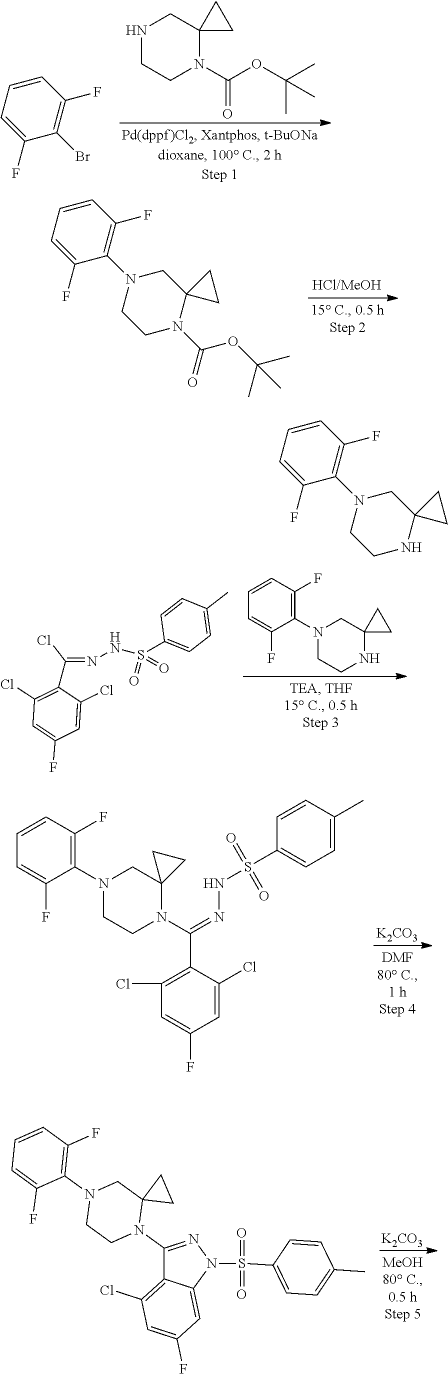

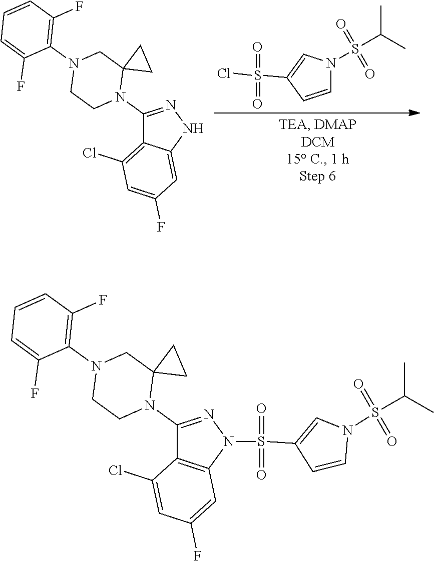 [SMILES: Fc1cc(Cl)c2c(N3CCN(c4c(F)cccc4F)CC34CC4)n[nH]c2c1 ; CC(C)S(=O)(=O)n1ccc(S(=O)(=O)Cl)c1 ; CC(C)S(=O)(=O)n1ccc(S(=O)(=O)n2nc(N3CCN(c4c(F)cccc4F)CC34CC4)c3c(Cl)cc(F)cc32)c1]

Step 1: A mixture of 2-bromo-1, 3-difluoro-benzene (1 g, 5.18 mmol, 1 eq), tert-butyl 4,7-diazaspiro[2.5]octane-4-carboxylate (1.10 g, 5.18 mmol, 1 eq), t-BuONa (1.49 g, 15.5 mmol, 3 eq), Xantphos (360 mg, 622 μmol, 0.12 eq) and Pd(dppf)Cl₂ (227 mg, 311 μmol, 0.06 eq) in dioxane (2 mL) was degassed and purged with N₂ for 3 times, and then the mixture was stirred at 100° C. for 2 hours under N₂ atmosphere. LC-MS showed 2-bromo-1, 3-difluoro-benzene was consumed completely and desired mass was detected. The reaction mixture was added to water (40 mL), extracted with EtOAc (30 mL*3). The combined organic layers were washed with brine (40 mL) and dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/petroleum ether gradient @80 mL/min) to give desired tert-butyl 7-(2, 6-difluorophenyl)-4, 7-diazaspiro [2.5]octane-4-carboxylate (300 mg, 925 μmol, 17.9% yield) as a brown oil. MS (ESI): mass calcd. For C₁₇H₂₂F₂N₂O₂ 324.16, m/z found 325.1 [M+H]⁺.

Step 2: To a solution of tert-butyl 7-(2, 6-difluorophenyl)-4, 7-diazaspiro [2.5]octane-4-carboxylate (300 mg, 925 μmol, 1 eq) in HCl/MeOH (3 mL). The mixture was stirred at 15° C. for 0.5 hour. TLC (petroleum ether/Ethyl acetate=3/1) showed tert-butyl 7-(2, 6-difluorophenyl)-4, 7-diazaspiro [2.5]octane-4-carboxylate was consumed completely and desired mass was detected. The reaction mixture was concentrated to give desired 7-(2, 6-difluorophenyl)-4, 7-diazaspiro [2.5]octane (300 mg, crude, HCl) as a yellow solid.

Step 3: To a solution of 7-(2, 6-difluorophenyl)-4, 7-diazaspiro [2.5]octane (283 mg, 1.26 mmol, 1 eq) in THF (5 mL) was added TEA (320 mg, 3.16 mmol, 440 μL, 2.5 eq) and (1Z)-2,6-dichloro-4-fluoro-N-(p-tolylsulfonyl)benzohydrazonoyl chloride (500 mg, 1.26 mmol, 1 eq). The mixture was stirred at 15° C. for 0.5 hour. LC-MS showed 7-(2, 6-difluorophenyl)-4, 7-diazaspiro [2.5]octane was consumed completely and one main peak with desired mass was detected. Then it was separated between water (20 mL) and ethyl acetate (15 mL*3). The organic phase was separated, washed with brine (20 mL), dried over Na₂SO₄, filtered and concentrated under reduced pressure to give desired (Z)—N′-((2,6-dichloro-4-fluorophenyl)(7-(2,6-difluorophenyl)-4,7-diazaspiro[2.5]octan-4-yl)methylene)-4-methylbenzenesulfonohydrazide (700 mg, crude) as a yellow oil. MS (ESI): mass calcd. For C₂₆H₂₃Cl₂F₃N₄O₂S 582.09, m/z found 583.2 [M+H]⁺.

Step 4: To a solution of N—[(Z)-[(2, 6-dichloro-4-fluoro-phenyl)-[7-(2, 6-difluorophenyl)-4, 7-diazaspiro [2.5]octan-4-yl]methylene]amino]-4-methyl-benzenesulfonamide (700 mg, 1.20 mmol, 1 eq) in DMF (10 mL) was added K₂CO₃ (1.66 g, 12.00 mmol, 10 eq). The mixture was stirred at 80° C. for 1 hour. LC-MS showed N—[(Z)-[(2,6-dichloro-4-fluoro-phenyl)-[7-(2,6-difluorophenyl)-4,7-diazaspiro[2.5]octan-4-yl]methylene]amino]-4-methyl-benzenesulfonamide was consumed completely and desired mass was detected. The reaction mixture was added to water (20 mL), the aqueous phase was extracted with EtOAc (15 mL*3). The organic layer was dried over Na₂SO₄, filtered and the filtrate was concentrated to give desired 4-chloro-3-(7-(2,6-difluorophenyl)-4, 7-diazaspiro [2.5]octan-4-yl)-6-fluoro-1-tosyl-1H-indazole (600 mg, crude) as a yellow oil. MS (ESI): mass calcd. For C₂₆H₂₂ClF₃N₄O₂S 546.11, m/z found 547.2 [M+H]⁺.

Step 5: To a solution of 4-chloro-3-[7-(2, 6-difluorophenyl)-4, 7-diazaspiro [2.5]octan-4-yl]-6-fluoro-1-(p-tolylsulfonyl) indazole (0.6 g, 1.10 mmol, 1 eq) in MeOH (10 mL) was added K₂CO₃ (758 mg, 5.48 mmol, 5 eq). The mixture was stirred at 80° C. for 0.5 hour. LC-MS showed 4-chloro-3-[7-(2, 6-difluorophenyl)-4, 7-diazaspiro [2.5]octan-4-yl]-6-fluoro-1-(p-tolylsulfonyl) indazole was consumed completely and desired mass was detected. The reaction mixture was added to water (20 mL), the aqueous phase was extracted with EtOAc (15 mL*3). The organic layer was dried over Na₂SO₄, filtered and the filtrate was concentrated to give a residue. The residue was purified by prep-TLC (SiO₂, petroleum ether/Ethyl acetate=2/1) to give desired 4-chloro-3-[7-(2, 6-difluorophenyl)-4, 7-diazaspiro [2.5]octan-4-yl]-6-fluoro-1H-indazole (100 mg, 255 μmol, 23.2% yield) as a colorless oil. MS (ESI): mass calcd. For C₁₉H₁₆ClF₃N₄ 392.10, m/z found 393.1 [M+H]⁺.

Step 6: To a solution of 4-chloro-3-[7-(2, 6-difluorophenyl)-4, 7-diazaspiro [2.5]octan-4-yl]-6-fluoro-1H-indazole (50 mg, 127 μmol, 1 eq) and 1-isopropylsulfonylpyrrole-3-sulfonyl chloride (69.2 mg, 255 μmol, 2 eq) in DCM (2 mL) was added TEA (25.8 mg, 255 μmol, 35.4 μL, 2 eq) and DMAP (1.56 mg, 12.7 μmol, 0.1 eq). The mixture was stirred at 15° C. for 1 hour. LC-MS showed 4-chloro-3-[7-(2, 6-difluorophenyl)-4, 7-diazaspiro [2.5]octan-4-yl]-6-fluoro-1H-indazole was consumed completely and desired mass was detected. The reaction mixture was concentrated under reduced pressure to give a residue. The residue was purified by prep-HPLC (neutral condition; column: Phenomenex C18 75*30 mm*3 um; mobile phase: [water(FA)-ACN]; B %: 60%-95%, 8 min) to give desired 4-chloro-3-(7-(2, 6-difluorophenyl)-4, 7-diazaspiro [2.5]octan-4-yl)-6-fluoro-1-((1-(isopropylsulfonyl)-1H-pyrrol-3-yl) sulfonyl)-1H-indazole (9.8 mg, 15.4 μmol, 12.1% yield, 98.63% purity) as a white solid. ¹H NMR (400 MHz, DMSO-d₆) δ 8.03 (t, J=1.9 Hz, 1H), 7.79 (dd, J=2.1, 8.9 Hz, 1H), 7.50 (dd, J=2.1, 9.1 Hz, 1H), 7.36-7.32 (m, 1H), 7.08-6.97 (m, 3H), 6.57 (dd, J=1.8, 3.3 Hz, 1H), 3.92 (quin, J=6.8 Hz, 1H), 3.56 (br s, 2H), 3.11 (br s, 2H), 2.51-2.45 (m, 2H), 1.05 (d, J=6.8 Hz, 6H), 0.78 (br s, 2H), 0.49 (br s, 2H). HPLC: 98.63% (220 nm), 99.08% (215 nm), 98.74% (254 nm). MS (ESI): mass calcd. For C₂₆H₂₅ClF₃N₅O₄S₂ 627.10, m/z found 628.2 [M+H]⁺.

### Compound 183: 4-chloro-6-fluoro-1-((1-(isopropylsulfonyl)-1H-pyrrol-3-yl) sulfonyl)-3-(7-(methylsulfonyl)-4, 7-diazaspiro [2.5]octan-4-yl)-1H-indazole

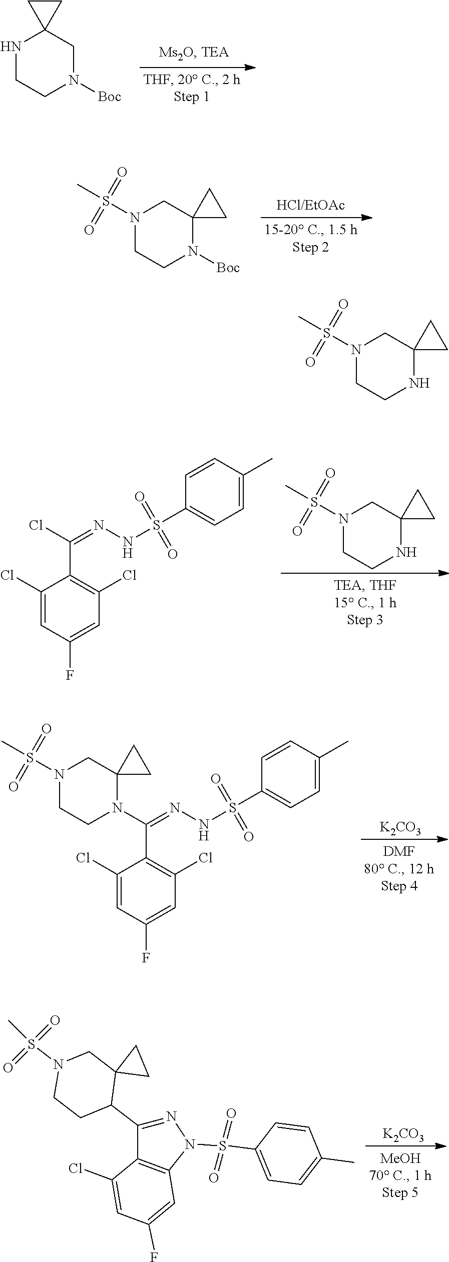

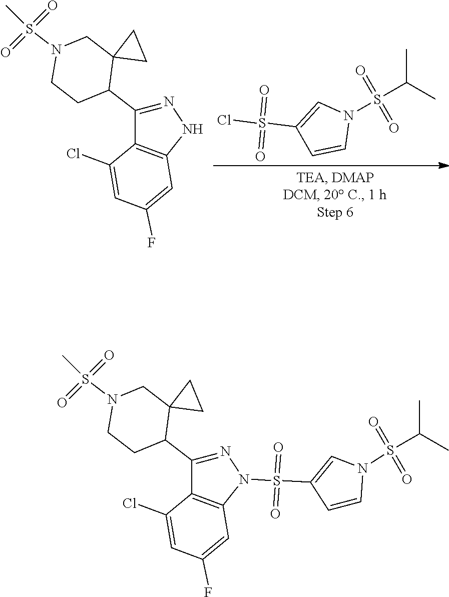

Step 1: To the solution of tert-butyl 4, 7-diazaspiro [2.5]octane-4-carboxylate (200 mg, 942 μmol, 1 eq) and TEA (286 mg, 2.83 mmol, 3 eq) in THF (2 mL) was added methylsulfonyl methanesulfonate (197 mg, 1.13 mmol, 1.2 eq) at 20° C. and the solution was stirred at 20° C. for 2 hours. TLC showed tert-butyl 4, 7-diazaspiro [2.5]octane-4-carboxylate was consumed completely and a new spot was formed. The reaction was quenched with NH₄Cl solution (1 mL) and the stirred for 15 min. The mixture was extracted with EtOAc (50 mL). The combined organics were dried over anhydrous Na₂SO₄ and concentrated to give desired tert-butyl 7-methylsulfonyl-4, 7-diazaspiro [2.5]octane-4-carboxylate (273 mg, crude) as a white solid.

Step 2: The solution of tert-butyl 7-methylsulfonyl-4, 7-diazaspiro [2.5]octane-4-carboxylate (273 mg, 940 μmol, 1 eq) in HCl/EtOAc (4 M, 3 mL, 12.8 eq) was stirred at 20° C. for 1 hour. To the reaction mixture was added 7-methylsulfonyl-4, 7-diazaspiro [2.5]octane (213 mg, 940 μmol, 1 eq, HCl) at 15° C. and the solution was stirred at 15° C. for 0.5 hour. TLC showed tert-butyl 7-methylsulfonyl-4, 7-diazaspiro [2.5]octane-4-carboxylate was consumed completely. The reaction mixture was concentrated to give desired 7-methylsulfonyl-4, 7-diazaspiro [2.5]octane (213 mg, crude, HCl) as a white solid.

Step 3: To the solution of (1Z)-2,6-dichloro-4-fluoro-N-(p-tolylsulfonyl)benzohydrazonoyl chloride (371 mg, 939 μmol, 1 eq) in THF (10 mL) was added TEA (951 mg, 9.39 mmol, 10 eq) at 15° C. and the solution was stirred at 15° C. for 0.5 hour. To the reaction mixture was added 7-methylsulfonyl-4, 7-diazaspiro [2.5]octane (213 mg, 940 μmol, 1 eq, HCl) at 15° C. and the solution was stirred at 15° C. for 0.5 hour. LCMS showed (1Z)-2, 6-dichloro-4-fluoro-N-(p-tolylsulfonyl) benzohydrazonoyl chloride was consumed completely and desired mass was detected. The reaction mixture was concentrated to give desired N-[(E)-[(2, 6-dichloro-4-fluoro-phenyl)-(7-methylsulfonyl-4, 7-diazaspiro [2.5]octan-4-yl) methylene]amino]-4-methyl-benzenesulfonamide (516 mg, crude) as a yellow solid. MS (ESI): mass calcd. For C₂₁H₂₃N₄Cl₂S₂O₄F 548.1, m/z found 549.1 [M+H]⁺.

Step 4: To the solution of N-[(E)-[(2, 6-dichloro-4-fluoro-phenyl)-(7-methylsulfonyl-4, 7-diazaspiro [2.5]octan-4-yl) methylene]amino]-4-methyl-benzenesulfonamide (516 mg, 939 μmol, 1 eq) in DMF (5 mL) was added K₂CO₃ (1.30 g, 9.39 mmol, 10 eq) and the solution was stirred at 80° C. for 12 hours. LCMS showed N-[(E)-[(2, 6-dichloro-4-fluoro-phenyl)-(7-methylsulfonyl-4, 7-diazaspiro [2.5]octan-4-yl) methylene]amino]-4-methyl-benzenesulfonamide was consumed completely and desired mass was detected. The reaction was poured into water (30 mL) and extracted with MTBE (100 mL). The combined organics were concentrated to give desired 4-chloro-6-fluoro-3-(7-methylsulfonyl-4, 7-diazaspiro [2.5]octan-4-yl)-1-(p-tolylsulfonyl) indazole (481 mg, crude) as a yellow oil. MS (ESI): mass calcd. For C₂₁H₂₂N₄ClS₂O₄F 512.1, m/z found 513.2 [M+H]⁺.

Step 5: The reaction mixture of 4-chloro-6-fluoro-3-(7-methylsulfonyl-4, 7-diazaspiro [2.5]octan-4-yl)-1-(p-tolylsulfonyl) indazole (481 mg, 938 μmol, 1 eq) and K₂CO₃ (1.30 g, 9.38 mmol, 10 eq) in MeOH (5 mL) was stirred at 70° C. for 1 hour. LCMS showed 4-chloro-6-fluoro-3-(7-methylsulfonyl-4, 7-diazaspiro [2.5]octan-4-yl)-1-(p-tolylsulfonyl) indazole was consumed completely and desired mass was detected. The reaction was concentrated to get a residue. The residue was added water (5 mL) and extracted with MTBE (40 mL). The combined organics were concentrated to get a residue. The residue was purified by prep-TLC (SiO₂, petroleum ether/ethyl acetate=2/1) to give desired 4-chloro-6-fluoro-3-(7-methylsulfonyl-4, 7-diazaspiro [2.5]octan-4-yl)-1H-indazole (100 mg, 279 μmol, 29.7% yield) as a white solid. MS (ESI): mass calcd. For C₁₄H₁₆N₄ClFSO₂ 358.1 m/z found 359.1 [M+H]⁺.

Step 6: To the solution of 4-chloro-6-fluoro-3-(7-methylsulfonyl-4,7-diazaspiro[2.5]octan-4-yl)-1H-indazole (30 mg, 83.6 μmol, 1 eq), TEA (42.3 mg, 418 μmol, 5 eq) and DMAP (1.02 mg, 8.36 μmol, 0.1 eq) in DCM (2 mL) was added 1-isopropylsulfonylpyrrole-3-sulfonyl chloride (22.7 mg, 83.6 μmol, 1 eq) and the solution was stirred at 20° C. for 1 hour. LCMS showed 4-chloro-6-fluoro-3-(7-methylsulfonyl-4, 7-diazaspiro [2.5]octan-4-yl)-1H-indazole was consumed completely and desired mass was detected. The reaction was concentrated to get a residue. The residue was purified by prep-HPLC (column: Phenomenex Luna C18 75*30 mm*3 μm; mobile phase: [water(FA)-ACN]; B %: 50%-100%, 8 min) to give desired 4-chloro-6-fluoro-1-(1-isopropylsulfonylpyrrol-3-yl)sulfonyl-3-(7-methylsulfonyl-4,7-diazaspiro[2.5]octan-4-yl) indazole (18.9 mg, 31.4 μmol, 37.6% yield, 98.82% purity) as a white solid. ¹H NMR (400 MHz, DMSO-d₆) δ 8.06 (m, 1H), 7.80 (m, 1H), 7.54 (m, 1H), 7.38 (m, 1H), 6.62 (m, 1H), 3.87-4.13 (m, 2H), 3.55 (br s, 2H), 3.06-3.19 (m, 3H), 2.93 (s, 3H), 1.08 (d, 6H), 0.83 (br s, 2H), 0.53 (br s, 2H). HPLC: 98.82% (220 nm), 98.91% (215 nm), 98.61% (254 nm). MS (ESI): mass calcd. For C₂₁H₂₅N₅S₃O₆ClF 593.1 m/z found 594.2 [M+H]⁺.

### Compound 184: 4-chloro-6-fluoro-1-(1-isopropylsulfonylpyrrol-3-yl)sulfonyl-3-[2-(trifluoromethyl)pyrrolidin-1-yl]indazole

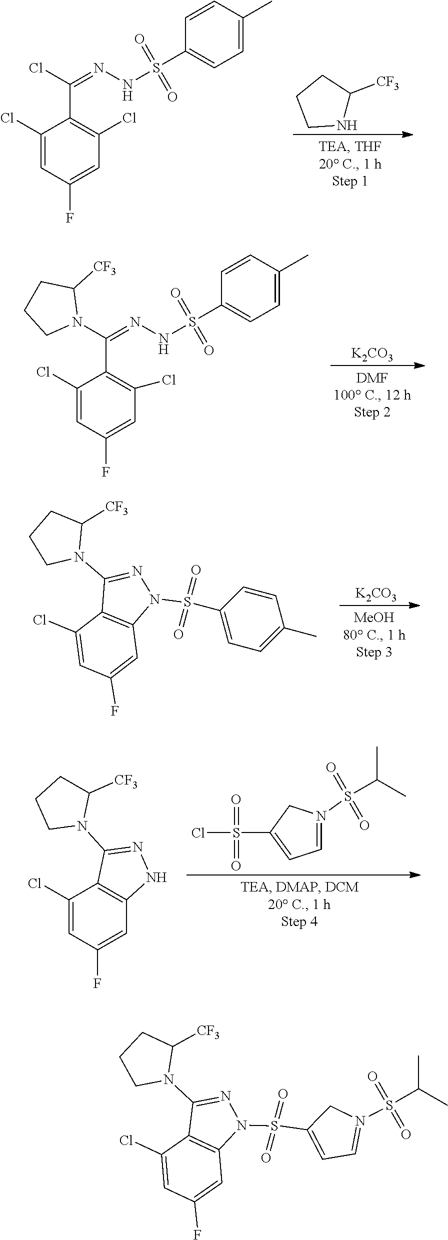

Step 1: To a solution of (1E)-2,6-dichloro-4-fluoro-N-(p-tolylsulfonyl)benzohydrazonoyl chloride (853 mg, 2.16 mmol, 1.5 eq) and 2-(trifluoromethyl)pyrrolidine (200 mg, 1.44 mmol, 1 eq) in THF (10 mL) was added TEA (364 mg, 3.59 mmol, 500 μL, 2.5 eq). The mixture was stirred at 20° C. for 1 hour. LC-MS showed (1E)-2, 6-dichloro-4-fluoro-N-(p-tolylsulfonyl) benzohydrazonoyl chloride was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired N-[(E)-[(2, 6-dichloro-4-fluoro-phenyl)-[2-(trifluoromethyl)pyrrolidin-1-yl]methylene]amino]-4-methyl-benzenesulfonamide (1.25 g, crude) as a yellow solid. MS (ESI): mass calcd. For C₁₉H₁₇Cl₂F₄N₃O₂S 497.0 m/z found 498.0 [M+H]⁺.

Step 2: To a solution of N-[(E)-[(2, 6-dichloro-4-fluoro-phenyl)-[2-(trifluoromethyl) pyrrolidin-1-yl]methylene]amino]-4-methyl-benzenesulfonamide (1.25 g, 2.51 mmol, 1 eq) in DMF (13 mL) was added K₂CO₃ (3.47 g, 25.1 mmol, 10 eq). The mixture was stirred at 100° C. for 12 hours. LC-MS showed N-[(E)-[(2, 6-dichloro-4-fluoro-phenyl)-[2-(trifluoromethyl) pyrrolidin-1-yl]methylene]amino]-4-methyl-benzenesulfonamide was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give desired 4-chloro-6-fluoro-1-(p-tolylsulfonyl)-3-[2-(trifluoromethyl)pyrrolidin-1-yl]indazole (1.16 g, crude) as a yellow solid. MS (ESI): mass calcd. For C₁₉H₁₆ClF₄N₃O₂S 461.1, m/z found 462.1 [M+H]⁺.

Step 3: To a solution of 4-chloro-6-fluoro-1-(p-tolylsulfonyl)-3-[2-(trifluoromethyl) pyrrolidin-1-yl]indazole (1.16 g, 2.51 mmol, 1 eq) in MeOH (12 mL) was added K₂CO₃ (1.74 g, 12.6 mmol, 5 eq). The mixture was stirred at 80° C. for 1 hour. LC-MS showed 4-chloro-6-fluoro-1-(p-tolylsulfonyl)-3-[2-(trifluoromethyl) pyrrolidin-1-yl]indazole was consumed completely and desired mass was detected. The reaction mixture was partitioned between 10 mL of H₂O and 10 mL of EtOAc. The organic phase was separated, washed with 50 mL of brine, dried over Na₂SO₄, filtered and concentrated under reduced pressure to give the crude product. The residue was purified by prep-TLC (SiO₂, petroleum ether:EtOAc=3:1) to give desired 4-chloro-6-fluoro-3-[2-(trifluoromethyl)pyrrolidin-1-yl]-1H-indazole (65 mg, 211 μmol, 8.41% yield) was obtained as a yellow solid. MS (ESI): mass calcd. For Cl₂H₁₀ClF₄N₃ 307.1, m/z found 308.1 [M+H]⁺.

Step 4: To a solution of 4-chloro-6-fluoro-3-[2-(trifluoromethyl) pyrrolidin-1-yl]-1H-indazole (30 mg, 97.5 μmol, 1 eq) and 1-isopropylsulfonylpyrrole-3-sulfonyl chloride (39.8 mg, 146 μmol, 1.5 eq) in DCM (1 mL) was added TEA (19.7 mg, 195 μmol, 27.1 μL, 2 eq) and DMAP (1.19 mg, 9.75 μmol, 0.1 eq). The mixture was stirred at 20° C. for 1 hour. LC-MS showed 4-chloro-6-fluoro-3-[2-(trifluoromethyl) pyrrolidin-1-yl]-1H-indazole was consumed completely and desired mass was detected. The reaction mixture was partitioned between 10 mL of H₂O and 10 mL of DCM. The organic phase was separated, washed with 50 mL of brine, dried over Na₂SO₄, filtered and concentrated under reduced pressure to give the crude product. The residue was purified by prep-HPLC (neutral condition; according to LCMS. Method: column: Waters Xbridge BEH C18 100*30 mm*10 um; mobile phase: [water(NH₄HCO₃)-ACN]; B %: 60%-90%, 8 min to give desired 4-chloro-6-fluoro-1-(1-isopropylsulfonylpyrrol-3-yl)sulfonyl-3-[2-(trifluoromethyl)pyrrolidin-1-yl]indazole (3 mg, 5.32 μmol, 5.46% yield, 96.3% purity) as a white solid. ¹H NMR (400 MHz, DMSO-d₆) δ 8.06-8.01 (m, 1H), 7.82 (dd, J=1.5, 8.8 Hz, 1H), 7.57 (dd, J=1.9, 9.1 Hz, 1H), 7.35 (t, J=2.7 Hz, 1H), 6.59-6.51 (m, 1H), 5.15-5.04 (m, 1H), 3.97-3.87 (m, 1H), 3.80-3.70 (m, 1H), 3.32 (br s, 1H), 2.46-2.33 (m, 1H), 2.00-1.91 (m, 2H), 1.84-1.74 (m, 1H), 1.07 (dd, J=3.0, 6.7 Hz, 6H). HPLC: 96.35% (220 nm), 94.76% (210 nm), 99.26% (254 nm). MS (ESI): mass calcd. For C₁₉H₂₀ClF₄N₄O₄S₂ 543.1, m/z found 544.1 [M+H]⁺.

### Compound 185: 4-chloro-6-fluoro-3-[4-(5-fluoropyrimidin-2-yl)-2, 2-dimethyl-piperazin-1-yl]-1-(1-isopropylsulfonylpyrrol-3-yl) sulfonyl-indazole

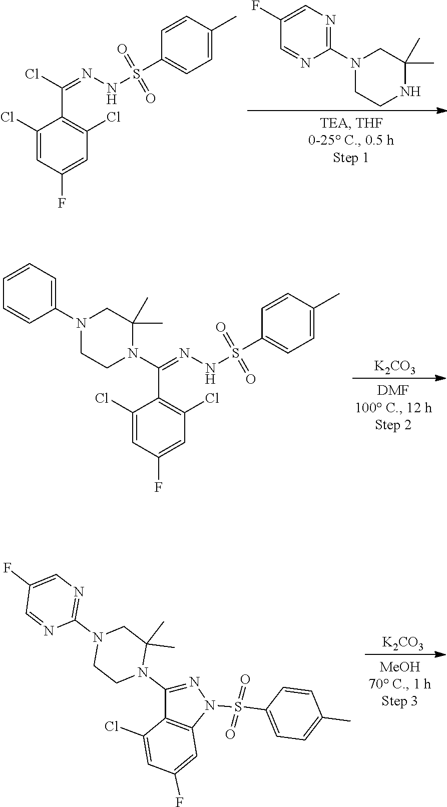

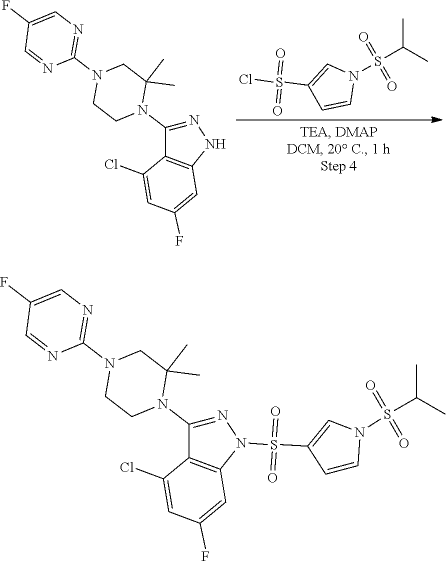 [SMILES: CC1(C)CN(c2ncc(F)cn2)CCN1c1n[nH]c2cc(F)cc(Cl)c12 ; CC(C)S(=O)(=O)n1ccc(S(=O)(=O)Cl)c1 ; CC(C)S(=O)(=O)n1ccc(S(=O)(=O)n2nc(N3CCN(c4ncc(F)cn4)CC3(C)C)c3c(Cl)cc(F)cc32)c1]

Step 1: To a solution of 2-(3, 3-dimethylpiperazin-1-yl)-5-fluoro-pyrimidine (200 mg, 811 μmol, 1 eq, HCl) in THF (3 mL) was added dropwise TEA (820 mg, 8.11 mmol, 1.13 mL, 10 eq) at 25° C. After addition, the mixture was stirred at this temperature for 10 mins, and then (1Z)-2, 6-dichloro-4-fluoro-N-(p-tolylsulfonyl)benzohydrazonoyl chloride (353 mg, 892 μmol, 1.1 eq) in THF (3 mL) was added dropwise at 0° C. The resulting mixture was stirred at 25° C. for 20 mins. LC-MS showed 2-(3, 3-dimethylpiperazin-1-yl)-5-fluoro-pyrimidine was consumed completely and one main peak with desired mass was detected. Then it was separated between 20 mL of water and 40 mL of EtOAc. The organic phase was separated, washed with 30 mL of brine, dried over Na₂SO₄, filtered and concentrated under reduced pressure to give desired N-[(E)-[(2, 6-dichloro-4-fluoro-phenyl)-[4-(5-fluoropyrimidin-2-yl)-2, 2-dimethyl-piperazin-1-yl]methylene]amino]-4-methyl-benzenesulfonamide (500 mg, crude) as a yellow solid. MS (ESI): mass calcd. For C₂₄H₂₄Cl₂F₂N₆O₂S 568.1, m/z found 569.1[M+H]⁺.

Step 2: To a solution of N-[(E)-[(2, 6-dichloro-4-fluoro-phenyl)-[4-(5-fluoropyrimidin-2-yl)-2, 2-dimethyl-piperazin-1-yl]methylene]amino]-4-methyl-benzenesulfonamide (500 mg, 878 μmol, 1 eq) in DMF (10 mL) was added K₂CO₃ (485 mg, 3.51 mmol, 4 eq). The mixture was stirred at 100° C. for 12 hours. LC-MS showed N-[(E)-[(2, 6-dichloro-4-fluoro-phenyl)-[4-(5-fluoropyrimidin-2-yl)-2, 2-dimethyl-piperazin-1-yl]methylene]amino]-4-methyl-benzenesulfonamide was consumed completely and desired mass was detected. The reaction mixture was added to water (20 mL), extracted with EtOAc (10 mL*3). The combined organic layers were washed with brine 20 mL and dried over Na₂SO₄, filtered and concentrated under reduced pressure to give desired 4-chloro-6-fluoro-3-[4-(5-fluoropyrimidin-2-yl)-2, 2-dimethyl-piperazin-1-yl]-1-(p-tolylsulfonyl) indazole (460 mg, crude) as an orange oil. MS (ESI): mass calcd. For C₂₄H₂₃ClF₂N₆O₂S 532.1, m/z found 533.2 [M+H]⁺.

Step 3: To a solution of 4-chloro-6-fluoro-3-[4-(5-fluoropyrimidin-2-yl)-2, 2-dimethyl-piperazin-1-yl]-1-(p-tolylsulfonyl) indazole (460 mg, 863 μmol, 1 eq) in MeOH (10 mL) was added K₂CO₃ (596 mg, 4.32 mmol, 5 eq). The mixture was stirred at 70° C. for 1 hour. LC-MS showed 4-chloro-6-fluoro-3-[4-(5-fluoropyrimidin-2-yl)-2, 2-dimethyl-piperazin-1-yl]-1-(p-tolylsulfonyl) indazole was consumed completely and desired compound was detected. The reaction mixture was added to water (20 mL), extracted with EtOAc (10 mL*3). The combined organic layers were washed with brine 20 mL and dried over Na₂SO₄, filtered and concentrated under reduced pressure to give a residue. The residue was purified by column chromatography (SiO₂, petroleum ether/EtOAc=1/0 to 1/1) to give desired 4-chloro-6-fluoro-3-[4-(5-fluoropyrimidin-2-yl)-2, 2-dimethyl-piperazin-1-yl]-1H-indazole (200 mg, crude) as a yellow oil. MS (ESI): mass calcd. For C₁₇H₁₇ClF₂N₆ 378.1, m/z found 379.0 [M+H]⁺.

Step 4: To a solution of 1-isopropylsulfonylpyrrole-3-sulfonyl chloride (53.8 mg, 198 μmol, 1.5 eq) in DCM (2 mL) was added TEA (40.1 mg, 396 μmol, 55.1 μL, 3 eq), 4-chloro-6-fluoro-3-[4-(5-fluoropyrimidin-2-yl)-2, 2-dimethyl-piperazin-1-yl]-1H-indazole (50 mg, 132 μmol, 1 eq) and DMAP (1.61 mg, 13.2 μmol, 0.1 eq). The mixture was stirred at 20° C. for 1 hour. LC-MS showed 4-chloro-6-fluoro-3-[4-(5-fluoropyrimidin-2-yl)-2, 2-dimethyl-piperazin-1-yl]-1H-indazole was consumed completely and desired mass was detected. The reaction mixture was concentrated to give the crude product. The residue was purified by prep-HPLC (neutral condition; column: Waters Xbridge BEH C18 100*30 mm*10 μm; mobile phase: [water(NH₄HCO₃)-ACN]; B %: 70%-95%, 8 min) to give desired 4-chloro-6-fluoro-3-[4-(5-fluoropyrimidin-2-yl)-2, 2-dimethyl-piperazin-1-yl]-1-(1-isopropylsulfonylpyrrol-3-yl)sulfonyl-indazole (3.0 mg, 4.83 μmol, 3.66% yield, 98.84% purity) as a white solid. ¹H NMR (400 MHz, DMSO-d₆) δ 8.46 (s, 2H), 8.12 (t, J=1.9 Hz, 1H), 7.85 (dd, J=2.1, 8.9 Hz, 1H), 7.55 (dd, J=2.1, 9.1 Hz, 1H), 7.39 (dd, J=2.4, 3.3 Hz, 1H), 6.60 (dd, J=1.6, 3.4 Hz, 1H), 4.04-3.83 (m, 3H), 3.66 (br s, 2H), 3.23 (br t, J=4.9 Hz, 2H), 1.23 (s, 6H), 1.13 (s, 3H), 1.11 (s, 3H). HPLC: 98.84% (220 nm), 98.95% (215 nm), 99.63% (254 nm). MS (ESI): mass calcd. For C₂₄H₂₆ClF₂N₇O₄S₂ 613.1, m/z found 614.1 [M+H]⁺.

### Compound 186: 4-[4-chloro-6-fluoro-1-(1-isopropylsulfonylpyrrol-3-yl) sulfonyl-indazol-3-yl]-N, N-dimethyl-4-azaspiro [2.5]octan-7-amine

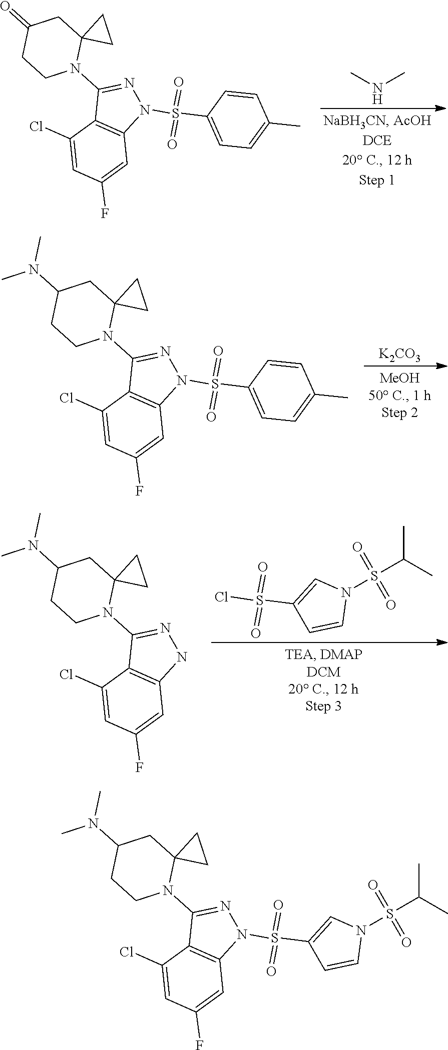

Step 1: To a solution of 4-[4-chloro-6-fluoro-1-(p-tolylsulfonyl) indazol-3-yl]-4-azaspiro [2.5]octan-7-one (50 mg, 112 μmol, 1 eq) in DCE (1 mL) was added N-methylmethanamine (2 M, 72.6 μL, 1.3 eq) and sodium; triacetoxyboranuide (35.5 mg, 167 μmol, 1.5 eq). Then AcOH (20.1 mg, 335 μmol, 19.2 μL, 3 eq) was added. The mixture was stirred at 20° C. for 12 hours. LC-MS showed 4-[4-chloro-6-fluoro-1-(p-tolylsulfonyl) indazol-3-yl]-4-azaspiro [2.5]octan-7-one was consumed completely and one main peak with desired mass was detected. The reaction mixture was quenched by addition sat.sodium carbonate (5 mL) at 0° C., and then diluted with H₂O (5 mL) and extracted with DCM (20 mL). The combined organic layers were washed with brine (10 mL), dried over anhydrous Na₂SO₄, filtered and concentrated under reduced pressure to give desired 4-[4-chloro-6-fluoro-1-(p-tolylsulfonyl) indazol-3-yl]-N, N-dimethyl-4-azaspiro[2.5]octan-7-amine (53 mg, crude) as a yellow oil. MS (ESI): mass calcd. For C₂₃H₂₆ClFN₄O₂S 476.1 m/z found 477.2 [M+H]⁺.

Step 2: To a solution of 4-[4-chloro-6-fluoro-1-(p-tolylsulfonyl) indazol-3-yl]-N, N-dimethyl-4-azaspiro [2.5]octan-7-amine (53 mg, 111 μmol, 1 eq) in MeOH (2 mL) was added K₂CO₃ (76.8 mg, 556 μmol, 5 eq). The mixture was stirred at 50° C. for 1 hour. LC-MS showed 4-[4-chloro-6-fluoro-1-(p-tolylsulfonyl) indazol-3-yl]-N, N-dimethyl-4-azaspiro [2.5]octan-7-amine was consumed completely and one main peak with desired mass was detected. The reaction mixture was concentrated under reduced pressure to remove MeOH. The residue was diluted with H₂O (5 mL) and extracted with EtOAc (10 mL). The combined organic layers were washed with brine (10 mL), dried over anhydrous Na₂SO₄, filtered and concentrated under reduced pressure to give a residue. The residue was purified by prep-HPLC (neutral condition: column: Waters Xbridge Prep OBD C18 150*40 mm*10 μm; mobile phase: [water(NH₄HCO₃)-ACN]; B %: 25%-55%, 8 min) to give desired 4-(4-chloro-6-fluoro-1H-indazol-3-yl)-N,N-dimethyl-4-azaspiro [2.5]octan-7-amine (25 mg, 77.5 μmol, 69.7% yield) as a yellow oil which was confirmed by LC-MS. MS (ESI): mass calcd. For C₁₆H₂₀ClFN₄ 322.1 m/z found 323.1 [M+H]⁺.

Step 3: To a solution of 4-(4-chloro-6-fluoro-1H-indazol-3-yl)-N, N-dimethyl-4-azaspiro [2.5]octan-7-amine (25 mg, 77.5 μmol, 1 eq) and 1-isopropylsulfonylpyrrole-3-sulfonyl chloride (42.1 mg, 155 μmol, 2 eq) in DCM (1 mL) was added TEA (23.5 mg, 232 μmol, 32.3 μL, 3 eq) and DMAP (946 μg, 7.74 μmol, 0.1 eq). The mixture was stirred at 20° C. for 12 hours. LC-MS showed 4-(4-chloro-6-fluoro-1H-indazol-3-yl)-N, N-dimethyl-4-azaspiro [2.5]octan-7-amine was consumed completely and one main peak with desired mass was detected. The reaction mixture was diluted with H₂O (10 mL) and extracted with DCM (10 mL). The combined organic layers were washed with brine (10 mL), dried over anhydrous Na₂SO₄, filtered and concentrated under reduced pressure to give a residue. The residue was purified by prep-HPLC (neutral condition: column: Phenomenex C18 80*40 mm*3 m; mobile phase: [water (NH₄HCO₃)-ACN]; B %: 40%-80%, 8 min) to give desired 4-[4-chloro-6-fluoro-1-(1-isopropylsulfonylpyrrol-3-yl)sulfonyl-indazol-3-yl]-N, N-dimethyl-4-azaspiro [2.5]octan-7-amine (6.0 mg, 10.4 μmol, 13.4% yield, 96.23% purity) as a white solid. ¹H NMR (400 MHz, DMSO-d₆) δ 8.00 (t, J=1.69 Hz, 1H), 7.78 (dd, J=8.94, 2.06 Hz, 1H), 7.50 (dd, J=9.07, 1.94 Hz, 1H), 7.35 (t, J=2.88 Hz, 1H), 6.54 (dd, J=3.31, 1.56 Hz, 1H), 3.94 (quin, J=6.69 Hz, 1H), 3.60 (br d, J=13.38 Hz, 1H), 3.22 (br t, J=12.26 Hz, 1H), 2.31-2.48 (m, 2H), 2.16 (s, 6H), 1.70 (br d, J=13.26 Hz, 1H), 1.19-1.30 (m, 1H), 1.12 (br s, 1H), 1.08 (t, J=6.57 Hz, 6H), 0.69 (dt, J=8.85, 6.02 Hz, 1H), 0.40-0.52 (m, 2H), 0.28-0.37 (m, 1H). HPLC: 94.58% (220 nm), 94.36% (215 nm), 96.23% (254 nm). MS (ESI): mass calcd. For C₂₃H₂₉ClFN₅O₄S₂ 557.1 m/z found 558.1 [M+H]⁺.

### Compound 187: 4-[4-chloro-6-fluoro-1-(1-isopropylsulfonylpyrrol-3-yl) sulfonyl-indazol-3-yl]-4-azaspiro [2.5]octan-7-one

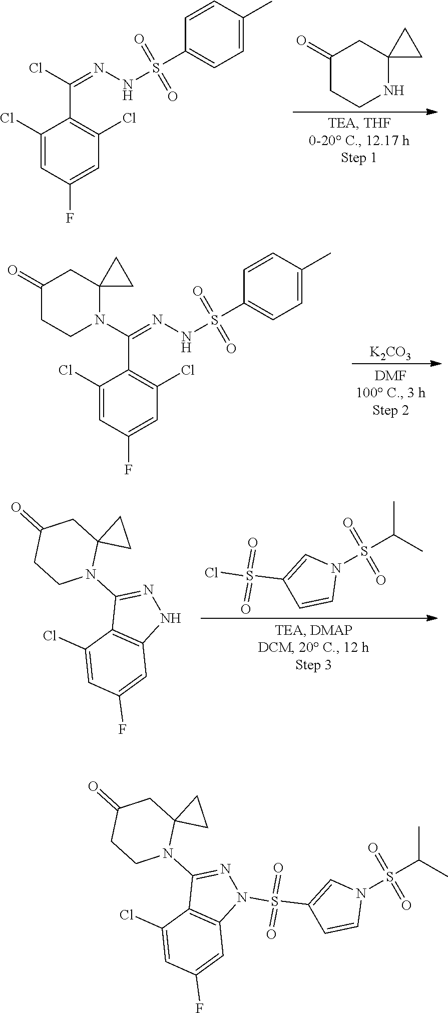

Step 1: To a solution of 4-azaspiro [2.5]octan-7-one (1.35 g, 8.34 mmol, 1.1 eq, HCl) in THF (10 mL) was added TEA (767 mg, 7.58 mmol, 1.06 mL, 1 eq). The mixture was stirred at 0° C. for 10 mins. Then (1E)-2,6-dichloro-4-fluoro-N-(p-tolylsulfonyl)benzohydrazonoyl chloride (3 g, 7.58 mmol, 1 eq) was added to the mixture. The mixture was stirred at 20° C. for 12 hours. LC-MS showed (1E)-2, 6-dichloro-4-fluoro-N-(p-tolylsulfonyl) benzohydrazonoyl chloride was consumed completely and one main peak with desired mass was detected. The reaction mixture was diluted with H₂O (30 mL) and extracted with EtOAc (150 mL). The combined organic layers were washed with brine (40 mL), dried over anhydrous Na₂SO₄, filtered and concentrated under reduced pressure to give desired N-[(E)-[(2, 6-dichloro-4-fluoro-phenyl)-(7-oxo-4-azaspiro [2.5]octan-4-yl)methylene]amino]-4-methyl-benzenesulfonamide (3.7 g, crude) as a yellow solid. MS (ESI): mass calcd. For C₂₁H₂₀Cl₂FN₃O₃S 483.1 m/z found 484.0 [M+H]⁺.

Step 2: To a solution of N-[(E)-[(2, 6-dichloro-4-fluoro-phenyl)-(7-oxo-4-azaspiro [2.5]octan-4-yl) methylene]amino]-4-methyl-benzenesulfonamide (3.7 g, 7.64 mmol, 1 eq) in DMF (30 mL) was added K₂CO₃ (10.6 g, 76.4 mmol, 10 eq). The mixture was stirred at 100° C. for 3 hours. LC-MS showed N-[(E)-[(2, 6-dichloro-4-fluoro-phenyl)-(7-oxo-4-azaspiro [2.5]octan-4-yl) methylene]amino]-4-methyl-benzenesulfonamide was consumed completely and one main peak with desired mass was detected. The reaction mixture was diluted with H₂O (50 mL) and extracted with MTBE (300 mL). The combined organic layers were washed with brine (40 mL), dried over anhydrous Na₂SO₄, filtered and concentrated under reduced pressure to give a residue. The residue was purified by flash silica gel chromatography (ISCO®; 80 g SepaFlash® Silica Flash Column, Eluent of 0˜35% EtOAc/petroleum ether gradient @70 mL/min) to give desired 4-(4-chloro-6-fluoro-1H-indazol-3-yl)-4-azaspiro[2.5]octan-7-one (100 mg, 340 μmol, 4.46% yield) as a yellow solid which was confirmed by LC-MS. MS (ESI): mass calcd. For C₁₄H₁₃ClFN₃O 293.1 m/z found 294.0 [M+H]⁺.

Step 3: To a solution of 4-(4-chloro-6-fluoro-1H-indazol-3-yl)-4-azaspiro [2.5]octan-7-one (80 mg, 272 μmol, 1 eq) and 1-isopropylsulfonylpyrrole-3-sulfonyl chloride (148 mg, 545 μmol, 2 eq) in DCM (2 mL) was added TEA (82.7 mg, 817 μmol, 114 μL, 3 eq) and DMAP (3.33 mg, 27.2 μmol, 0.1 eq). The mixture was stirred at 20° C. for 12 hours. LC-MS showed 4-(4-chloro-6-fluoro-1H-indazol-3-yl)-4-azaspiro [2.5]octan-7-one was consumed completely and one main peak with desired mass was detected. The reaction mixture was diluted with H₂O (5 mL) and extracted with DCM (10 mL). The combined organic layers were washed with brine (10 mL), dried over anhydrous Na₂SO₄, filtered and concentrated under reduced pressure to give a residue. The residue was purified by prep-HPLC (neutral condition: column: Waters Xbridge BEH C18 100*30 mm*10 m; mobile phase: [water (NH₄HCO₃)-ACN]; B %: 55%-85%, 8 min) to give desired 4-[4-chloro-6-fluoro-1-(1-isopropylsulfonylpyrrol-3-yl) sulfonyl-indazol-3-yl]-4-azaspiro [2.5]octan-7-one (10 mg, 18.9 μmol, 6.94% yield, 100% purity) as a white solid. ¹H NMR (400 MHz, DMSO-d₆) δ 8.06 (t, J=1.75 Hz, 1H), 7.82 (dd, J=8.88, 2.00 Hz, 1H), 7.54 (dd, J=9.13, 2.00 Hz, 1H), 7.36 (dd, J=3.19, 2.44 Hz, 1H), 6.60 (dd, J=3.19, 1.56 Hz, 1H), 3.94 (dt, J=13.48, 6.71 Hz, 1H), 3.72 (br t, J=5.88 Hz, 2H), 2.70 (br s, 2H), 2.31 (br t, J=5.94 Hz, 2H), 1.08 (d, J=6.75 Hz, 6H), 0.64-0.69 (m, 2H), 0.48-0.53 (m, 2H). HPLC: 99.36% (220 nm), 97.94% (215 nm), 100.0% (254 nm). MS (ESI): mass calcd. For C₂₁H₂₂ClFN₄O₅S₂ 528.1 m/z found 529.1 [M+H]⁺.

### Compound 188: 3-(4-azaspiro [2.4]heptan-4-yl)-4-chloro-6-fluoro-1-(1-isopropylsulfonylpyrrol-3-yl) sulfonyl-indazole

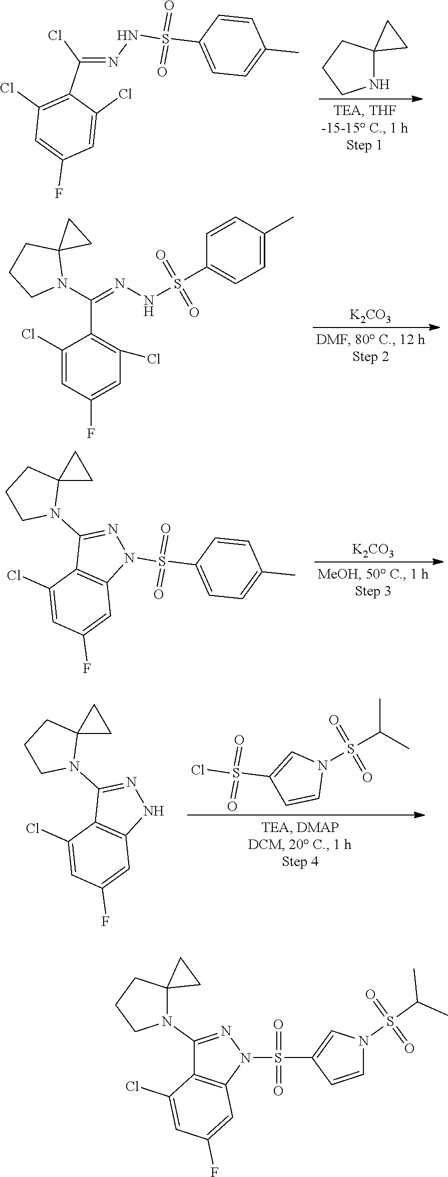

Step 1: To the solution of 4-azaspiro [2.4]heptane (0.15 g, 1.12 mmol, 1 eq, HCl) in THF (3 mL) was added TEA (1.13 g, 11.2 mmol, 1.56 mL, 10 eq) at 15° C. and the solution was stirred at 15° C. for 0.5 hour. To the reaction mixture was added (1E)-2, 6-dichloro-4-fluoro-N-(p-tolylsulfonyl)benzohydrazonoyl chloride (443 mg, 1.12 mmol, 1 eq) at −15° C. and the solution was stirred at 15° C. for 0.5 hour. LCMS showed (1E)-2, 6-dichloro-4-fluoro-N-(p-tolylsulfonyl) benzohydrazonoyl chloride was consumed completely and desired mass was detected. The reaction mixture was concentrated to give desired N-[(E)-[4-azaspiro [2.4]heptan-4-yl-(2, 6-dichloro-4-fluoro-phenyl) methylene]amino]-4 methyl-benzenesulfonamide (511 mg, crude) as a yellow solid. MS (ESI): mass calcd. For C₂₀H₂₀N₃Cl₂SO₂F 455.1 m/z found 456.1 [M+H]⁺.

Step 2: The mixture of N-[(E)-[4-azaspiro [2.4]heptan-4-yl-(2, 6-dichloro-4-fluoro-phenyl) methylene]amino]-4-methyl-benzenesulfonamide (511 mg, 1.12 mmol, 1 eq) and K₂CO₃ (1.55 g, 11.2 mmol, 10 eq) in DMF (5 mL) was stirred at 80° C. for 12 hours. LCMS showed N-[(E)-[4-azaspiro [2.4]heptan-4-yl-(2, 6-dichloro-4-fluoro-phenyl) methylene]amino]-4-methyl-benzenesulfonamide was consumed completely and desired mass was detected. The reaction was concentrated to get a residue. The residue was added water (5 mL) and extracted with MTBE (40 mL). The combined organics were concentrated to give desired 3-(4-azaspiro [2.4]heptan-4-yl)-4-chloro-6-fluoro-1-(p-tolylsulfonyl) indazole (470 mg, crude) was a yellow solid. MS (ESI): mass calcd. For C₂₀H₁₉N₃ClSO₂F 419.1 m/z found 420.2 [M+H]⁺.

Step 3: The mixture of 3-(4-azaspiro [2.4]heptan-4-yl)-4-chloro-6-fluoro-1-(p-tolylsulfonyl) indazole (470 mg, 1.12 mmol, 1 eq) and K₂CO₃ (464 mg, 3.36 mmol, 3 eq) in MeOH (5 mL) was stirred at 50° C. for 1 hour. LCMS showed 3-(4-azaspiro [2.4]heptan-4-yl)-4-chloro-6-fluoro-1-(p-tolylsulfonyl) indazole was consumed completely and desired mass was detected. The reaction was concentrated to get a residue. The residue was purified by prep-TLC (SiO₂, petroleum ether/ethyl acetate=2/1) to give desired 3-(4-azaspiro[2.4]heptan-4-yl)-4-chloro-6-fluoro-1H-indazole (130 mg, crude) as a yellow solid. MS (ESI): mass calcd. For C₁₃H₁₃N₃ClF 265.1 m/z found 266.1 [M+H]⁺.

Step 4: The mixture of 3-(4-azaspiro[2.4]heptan-4-yl)-4-chloro-6-fluoro-1H-indazole (40 mg, 151 μmol, 1 eq), TEA (76.2 mg, 753 μmol, 5 eq), DMAP (1.84 mg, 15.1 μmol, 0.1 eq) and 1-isopropylsulfonylpyrrole-3-sulfonyl chloride (49.1 mg, 181 μmol, 1.2 eq) in DCM (2 mL) was stirred at 20° C. for 1 hour. LCMS showed 3-(4-azaspiro [2.4]heptan-4-yl)-4-chloro-6-fluoro-1H-indazole was consumed completely and desired mass was detected. The reaction was concentrated to get a residue. The residue was purified by prep-HPLC (column: Waters Xbridge BEH C18 100*30 mm*10 μm; mobile phase: [water (NH₄HCO₃)-ACN]; B %: 60%-90%, 8 min) to give desired 3-(4-azaspiro [2.4]heptan-4-yl)-4-chloro-6-fluoro-1-(1-isopropylsulfonylpyrrol-3-yl)sulfonyl-indazole (6.0 mg, 11.8 μmol, 7.86% yield, 98.79% purity) as a white solid. ¹H NMR (DMSO-d₆) δ: 7.99 (s, 1H), 7.77 (m, 1H), 7.50 (m, 1H), 7.36 (m, 1H), 6.50-6.60 (m, 1H), 3.95 (dm, 1H), 3.44 (br s, 2H), 1.91 (br d, 4H), 1.09 (d, 6H), 0.69-0.79 (m, 2H), 0.55-0.64 (m, 2H). HPLC: 98.79% (220 nm), 98.73% (215 nm), 98.53% (254 nm). MS (ESI): mass calcd. For C₂₀H₂₂N₄S₂O₄ClF 500.1 m/z found 501.0 [M+H]⁺.

### Compound 189: 3-(1-azaspiro [3.3]heptan-1-yl)-4-chloro-6-fluoro-1-(1-isopropylsulfonylpyrrol-3-yl) sulfonyl-indazole

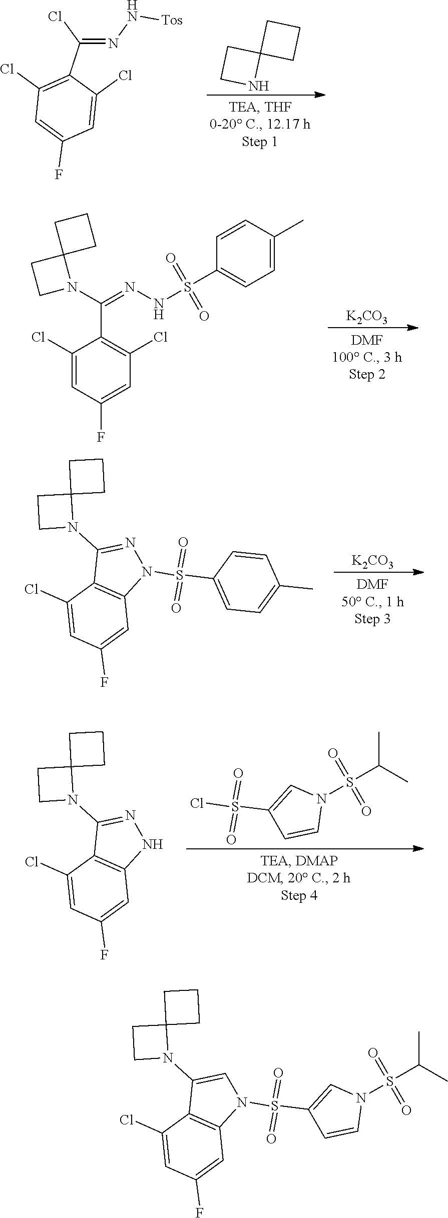

Step 1: To a solution of 1-azaspiro [3.3]heptane (150 mg, 1.12 mmol, 1 eq, HCl) in THF (5 mL) was added TEA (341 mg, 3.37 mmol, 469 μL, 3 eq). The mixture was stirred at 0° C. for 10 mins. Then (1Z)-2,6-dichloro-4-fluoro-N-(p-tolylsulfonyl) benzohydrazonoyl chloride (533 mg, 1.35 mmol, 1.2 eq) was added to the mixture. The mixture was stirred at 20° C. for 12 hours. LC-MS showed (1Z)-2, 6-dichloro-4-fluoro-N-(p-tolylsulfonyl) benzohydrazonoyl chloride was consumed completely and one main peak with desired mass was detected. The reaction mixture was diluted with H₂O (5 mL) and extracted with EtOAc (30 mL). The combined organic layers were washed with brine (20 mL), dried over anhydrous Na₂SO₄, filtered and concentrated under reduced pressure to give desired N-[(E)-[1-azaspiro [3.3]heptan-1-yl-(2, 6-dichloro-4-fluoro-phenyl) methylene]amino]-4-methyl-benzenesulfonamide (510 mg, crude) as a yellow oil. MS (ESI): mass calcd. For C₂₀H₂₀Cl₂FN₃O₂S 455.1 m/z found 456.0 [M+H]⁺.

Step 2: To a solution of N-[(E)-[1-azaspiro [3.3]heptan-1-yl-(2, 6-dichloro-4-fluoro-phenyl) methylene]amino]-4-methyl-benzenesulfonamide (510 mg, 1.12 mmol, 1 eq) in DMF (5 mL) was added K₂CO₃ (1.54 g, 11.2 mmol, 10 eq). The mixture was stirred at 100° C. for 3 hours. LC-MS showed N-[(E)-[1-azaspiro [3.3]heptan-1-yl-(2, 6-dichloro-4-fluoro-phenyl) methylene]amino]-4-methyl-benzenesulfonamide was consumed completely and one main peak with desired mass was detected. The reaction mixture was diluted with H₂O (20 mL) and extracted with MTBE (60 mL). The combined organic layers were washed with brine (20 mL), dried over anhydrous Na₂SO₄, filtered and concentrated under reduced pressure to give desired 3-(1-azaspiro[3.3]heptan-1-yl)-4-chloro-6-fluoro-1-(p-tolylsulfonyl) indazole (470 mg, crude) as a yellow oil. MS (ESI): mass calcd. For C₂₀H₁₉ClFN₃O₂S 419.1 m/z found 420.1 [M+H]⁺.

Step 3: To a solution of 3-(1-azaspiro [3.3]heptan-1-yl)-4-chloro-6-fluoro-1-(p-tolylsulfonyl) indazole (470 mg, 1.12 mmol, 1 eq) in MeOH (5 mL) was added K₂CO₃ (773 mg, 5.60 mmol, 5 eq). The mixture was stirred at 50° C. for 1 hour. LC-MS showed 3-(1-azaspiro [3.3]heptan-1-yl)-4-chloro-6-fluoro-1-(p-tolylsulfonyl) indazole was consumed completely and desired mass was detected. The reaction mixture was concentrated under reduced pressure to remove MeOH. The residue was diluted with H₂O (10 mL) and extracted with EtOAc (30 mL). The combined organic layers were washed with brine (20 mL), dried over anhydrous Na₂SO₄, filtered and concentrated under reduced pressure to give a residue. The residue was purified by prep-TLC (SiO₂, petroleum ether/EtOAc=2/1) to give desired 3-(1-azaspiro[3.3]heptan-1-yl)-4-chloro-6-fluoro-1H-indazole (40 mg, 151 μmol, 13.5% yield) as a yellow solid which was confirmed by LC-MS. MS (ESI): mass calcd. For C₁₃H₁₃ClFN₃ 265.1 m/z found 266.2 [M+H]⁺.

Step 4: To a solution of 3-(1-azaspiro [3.3]heptan-1-yl)-4-chloro-6-fluoro-1H-indazole (40 mg, 151 μmol, 1 eq) and 1-isopropylsulfonylpyrrole-3-sulfonyl chloride (61.4 mg, 226 μmol, 1.5 eq) in DCM (2 mL) was added TEA (45.7 mg, 452 μmol, 62.9 μL, 3 eq) and DMAP (1.84 mg, 15.1 μmol, 0.1 eq). The mixture was stirred at 20° C. for 2 hours. LC-MS showed 3-(1-azaspiro [3.3]heptan-1-yl)-4-chloro-6-fluoro-1H-indazole was consumed completely and one main peak with desired mass was detected. The reaction mixture was diluted with H₂O (5 mL) and extracted with DCM (10 mL). The combined organic layers were washed with brine (10 mL), dried over anhydrous Na₂SO₄, filtered and concentrated under reduced pressure to give a residue. The residue was purified by prep-HPLC (neutral condition: column: Waters Xbridge Prep OBD C18 150*40 mm*10 μm; mobile phase: [water(NH₄HCO₃)-ACN]; B %: 55%-75%, 8 min) to give desired 3-(1-azaspiro[3.3]heptan-1-yl)-4-chloro-6-fluoro-1-(1-isopropylsulfonylpyrrol-3-yl)sulfonyl-indazole (10 mg, 19.7 μmol, 13.1% yield, 98.9% purity) as a white solid. ¹H NMR (400 MHz, DMSO-d₆) δ 7.94 (t, J=1.94 Hz, 1H), 7.77 (dd, J=9.01, 2.13 Hz, 1H), 7.46 (dd, J=8.94, 2.19 Hz, 1H), 7.34-7.39 (m, 1H), 6.55 (dd, J=3.25, 1.63 Hz, 1H), 4.21 (t, J=7.19 Hz, 2H), 3.94 (spt, J=6.75 Hz, 1H), 2.98-3.10 (m, 2H), 2.38 (t, J=7.19 Hz, 2H), 1.89-1.99 (m, 2H), 1.77-1.87 (m, 1H), 1.55-1.68 (m, 1H), 1.10 (d, J=6.75 Hz, 6H). HPLC: 98.85% (220 nm), 98.91% (215 nm), 99.70% (254 nm). MS (ESI): mass calcd. For C₂₀H₂₂ClFN₄O₄S₂ 500.1 m/z found 501.0 [M+H]⁺.

### Compound 190: 4-[4-chloro-6-fluoro-1-(1-isopropylsulfonylpyrrol-3-yl) sulfonyl-indazol-3-yl]-7-oxa-4-azaspiro [2.5]octane

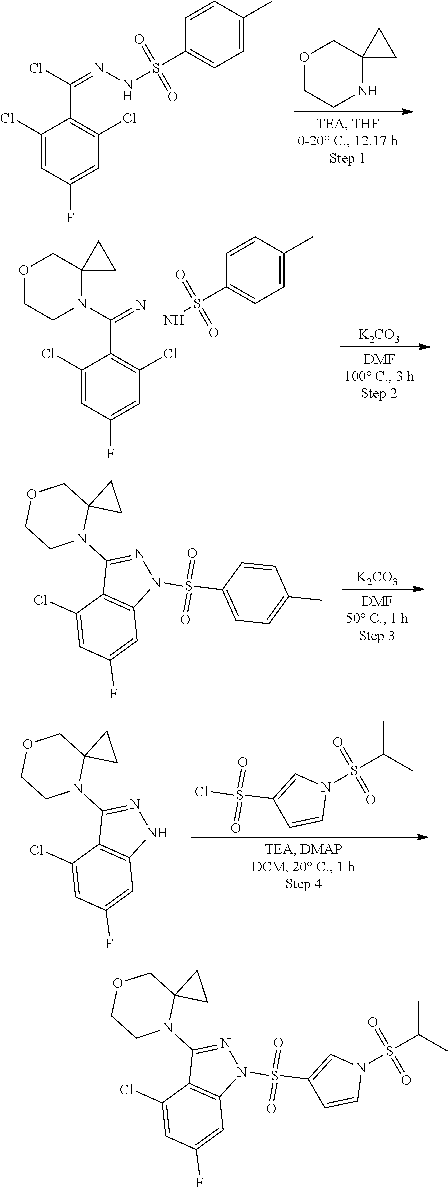

Step 1: To a solution of 7-oxa-4-azaspiro [2.5]octane (151 mg, 1.01 mmol, 1 eq, HCl) in THF (3 mL) was added TEA (307 mg, 3.03 mmol, 422 μL, 3 eq) at 20° C. The mixture was stirred at 0° C. for 10 mins. Then (1E)-2, 6-dichloro-4-fluoro-N-(p-tolylsulfonyl) benzohydrazonoyl chloride (400 mg, 1.01 mmol, 1 eq) in THF (3 mL) was added to the mixture. The mixture was stirred at 20° C. for 12 hours. LC-MS showed (1E)-2, 6-dichloro-4-fluoro-N-(p-tolylsulfonyl) benzohydrazonoyl chloride was consumed completely and one main peak with desired mass was detected. The reaction mixture was diluted with H₂O (10 mL) and extracted with EtOAc (20 mL). The combined organic layers were washed with brine (20 mL), dried over anhydrous Na₂SO₄, filtered and concentrated under reduced pressure to give desired N-[(E)-[(2, 6-dichloro-4-fluoro-phenyl)-(7-oxa-4-azaspiro [2.5]octan-4-yl) methylene]amino]-4-methyl-benzenesulfonamide (480 mg, crude) as a yellow solid. MS (ESI): mass calcd. For C₂₀H₂₀Cl₂FN₃O₃S 471.1 m/z found 472.1 [M+H]⁺.

Step 2: To a solution of N-[(E)-[(2, 6-dichloro-4-fluoro-phenyl)-(7-oxa-4-azaspiro [2.5]octan-4-yl) methylene]amino]-4-methyl-benzenesulfonamide (480 mg, 1.02 mmol, 1 eq) in DMF (5 mL) was added K₂CO₃ (1.40 g, 10.2 mmol, 10 eq). The mixture was stirred at 100° C. for 3 hours. LC-MS showed N-[(E)-[(2, 6-dichloro-4-fluoro-phenyl)-(7-oxa-4-azaspiro [2.5]octan-4-yl) methylene]amino]-4-methyl-benzenesulfonamide was consumed completely and one main peak with desired mass was detected. The reaction mixture was diluted with H₂O (20 mL) and extracted with MTBE (60 mL). The combined organic layers were washed with brine (20 mL), dried over anhydrous Na₂SO₄, filtered and concentrated under reduced pressure to give desired 4-[4-chloro-6-fluoro-1-(p-tolylsulfonyl) indazol-3-yl]-7-oxa-4-azaspiro [2.5]octane (440 mg, crude) as a yellow oil. MS (ESI): mass calcd. For C₂₀H₁₉ClFN₃O₃S 435.1 m/z found 436.1 [M+H]⁺.

Step 3: To a solution of 4-[4-chloro-6-fluoro-1-(p-tolylsulfonyl) indazol-3-yl]-7-oxa-4-azaspiro [2.5]octane (440 mg, 1.01 mmol, 1 eq) in MeOH (10 mL) was added K₂CO₃ (698 mg, 5.05 mmol, 5 eq). The mixture was stirred at 50° C. for 1 hour. LC-MS showed 4-[4-chloro-6-fluoro-1-(p-tolylsulfonyl) indazol-3-yl]-7-oxa-4-azaspiro [2.5]octane was consumed completely and desired mass was detected. The reaction mixture was concentrated under reduced pressure to remove MeOH. The residue was diluted with H₂O (10 mL) and extracted with EtOAc (20 mL). The combined organic layers were washed with brine (10 mL), dried over anhydrous Na₂SO₄, filtered and concentrated under reduced pressure to give a residue. The residue was purified by prep-TLC (SiO₂, petroleum ether/EtOAc=2/1) to give desired 4-(4-chloro-6-fluoro-1H-indazol-3-yl)-7-oxa-4-azaspiro[2.5]octane (80 mg, 284 μmol, 28.1% yield) as a yellow oil which was confirmed by LC-MS. MS (ESI): mass calcd. For C₁₃H₁₃ClFN₃O 281.1 m/z found 282.1 [M+H]⁺.

Step 4: To a solution of 4-(4-chloro-6-fluoro-1H-indazol-3-yl)-7-oxa-4-azaspiro [2.5]octane (40 mg, 142 μmol, 1 eq) and 1-isopropylsulfonylpyrrole-3-sulfonyl chloride (57.9 mg, 213 μmol, 1.5 eq) in DCM (2 mL) was added TEA (43.1 mg, 426 μmol, 59.3 μL, 3 eq) and DMAP (1.73 mg, 14.2 μmol, 0.1 eq). The mixture was stirred at 20° C. for 3 hours. LC-MS showed 4-(4-chloro-6-fluoro-1H-indazol-3-yl)-7-oxa-4-azaspiro [2.5]octane was consumed completely and one main peak with desired mass was detected. The reaction mixture was diluted with H₂O (5 mL) and extracted with DCM (10 mL). The combined organic layers were washed with brine (10 mL), dried over anhydrous Na₂SO₄, filtered and concentrated under reduced pressure to give a residue. The residue was purified by prep-HPLC (neutral condition: column: Waters Xbridge Prep OBD C18 150*40 mm*10 m; mobile phase: [water(NH₄HCO₃)-ACN]; B %: 40%-80%, 8 min) to give desired 4-[4-chloro-6-fluoro-1-(1-isopropylsulfonylpyrrol-3-yl) sulfonyl-indazol-3-yl]-7-oxa-4-azaspiro [2.5]octane (10 mg, 18.4 μmol, 12.9% yield, 94.9% purity) as a white solid. ¹H NMR (400 MHz, DMSO-d₆) δ 8.04 (s, 1H), 7.79 (dd, J=8.88, 1.75 Hz, 1H), 7.52 (dd, J=9.07, 1.69 Hz, 1H), 7.37 (t, J=2.63 Hz, 1H), 6.57 (br d, J=1.63 Hz, 1H), 3.95 (dt, J=13.45, 6.79 Hz, 1H), 3.72 (br s, 2H), 3.58 (br s, 2H), 3.46 (br d, J=3.88 Hz, 2H), 1.09 (d, J=6.75 Hz, 6H), 0.75 (s, 2H) 0.46 (br s, 2H). HPLC: 94.90% (220 nm), 94.96% (215 nm), 94.18% (254 nm). MS (ESI): mass calcd. For C₂₀H₂₂ClFN₄O₅S₂ 516.1 m/z found 517.1 [M+H]⁺.

### Compound 191: 4-chloro-3-(7, 7-difluoro-4-azaspiro [2.5]octan-4-yl)-6-fluoro-1-(1-isopropylsulfonylpyrrol-3-yl) sulfonyl-indazole

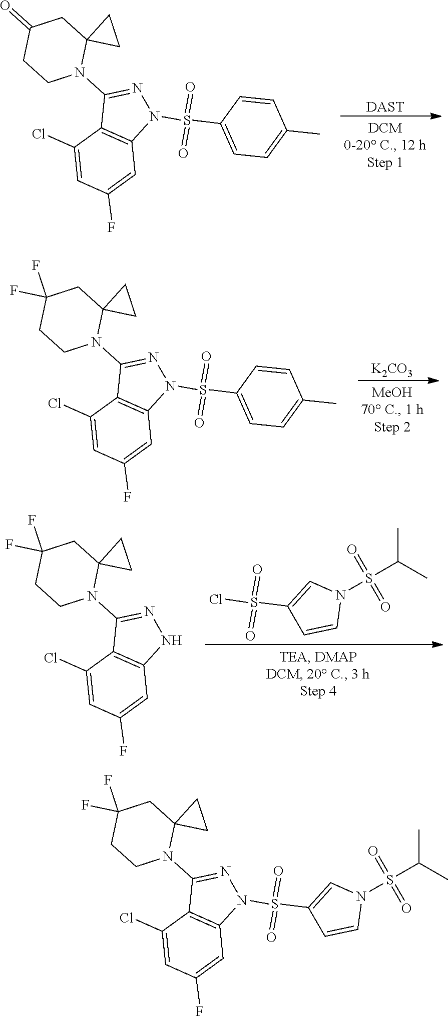

Step 1: To a solution of 4-[4-chloro-6-fluoro-1-(p-tolylsulfonyl) indazol-3-yl]-4 azaspiro [2.5]octan-7-one (100 mg, 223 μmol, 1 eq) in DCM (2 mL) was added DAST (180 mg, 1.12 mmol, 147 μL, 5 eq) dropwise at 0° C. The mixture was stirred at 0° C. for 1 hour. LCMS showed 4-[4-chloro-6-fluoro-1-(p-tolylsulfonyl) indazol-3-yl]-4-azaspiro [2.5]octan-7-one was consumed completely and one main peak with desired mass was detected. The reaction mixture was quenched by addition MeOH (5 mL) at 0° C., and then concentrated under reduced pressure to give a residue. The residue was purified by prep-TLC (SiO₂, petroleum ether/EtOAc=4/1) to give desired 4-chloro-3-(7, 7-difluoro-4-azaspiro [2.5]octan-4-yl)-6-fluoro-1-(p-tolylsulfonyl) indazole (80 mg, 170 μmol, 76.3% yield) as a yellow solid. MS (ESI): mass calcd. For C₂₁H₁₉ClF₃N₃O₂S 469.1 m/z found 470.1 [M+H]⁺.

Step 2: To a solution of 4-chloro-3-(7, 7-difluoro-4-azaspiro [2.5]octan-4-yl)-6 fluoro-1-(p-tolylsulfonyl) indazole (80 mg, 170 μmol, 1 eq) in MeOH (3 mL) was added K₂CO₃ (118 mg, 851 μmol, 5 eq). The mixture was stirred at 50° C. for 1 hour. LCMS showed 4-chloro-3-(7, 7-difluoro-4-azaspiro [2.5]octan-4-yl)-6-fluoro-1-(p-tolylsulfonyl) indazole was consumed completely and one main peak with desired mass was detected. The reaction mixture was concentrated under reduced pressure to remove MeOH. The residue was diluted with H₂O (5 mL) and extracted with EtOAc (20 mL). The combined organic layers were washed with brine (10 mL), dried over anhydrous Na₂SO₄, filtered and concentrated under reduced pressure to give a residue. The residue was purified by prep-TLC (SiO₂, petroleum ether/EtOAc=2/1) to give desired 4-chloro-3-(7, 7-difluoro-4-azaspiro [2.5]octan-4-yl)-6-fluoro-1H-indazole (30 mg, 95.0 μmol, 55.8% yield) as a pale yellow oil. MS (ESI): mass calcd. For C₁₄H₁₃ClF₃N₃ 315.1 m/z found 316.0 [M+H]⁺.

Step 3: To a solution of 4-chloro-3-(7, 7-difluoro-4-azaspiro [2.5]octan-4-yl)-6-fluoro-1H-indazole (30 mg, 95.0 μmol, 1 eq) and 1-isopropylsulfonylpyrrole-3-sulfonyl chloride (38.7 mg, 143 μmol, 1.5 eq) in DCM (2 mL) was added TEA (28.9 mg, 285 μmol, 39.7 μL, 3 eq) and DMAP (1.16 mg, 9.50 μmol, 0.1 eq). The mixture was stirred at 20° C. for 3 hours. LC-MS showed 4-chloro-3-(7, 7-difluoro-4-azaspiro [2.5]octan-4-yl)-6-fluoro-1H-indazole was consumed completely and one main peak with desired mass was detected. The reaction mixture was diluted with H₂O (5 mL) and extracted with DCM (10 mL). The combined organic layers were washed with brine (10 mL), dried over anhydrous Na₂SO₄, filtered and concentrated under reduced pressure to give a residue. The residue was purified by prep-HPLC (neutral condition: column: Waters Xbridge Prep OBD C18 150*40 mm*10 μm; mobile phase: [water (NH₄HCO₃)-ACN]; B %: 45%-85%, 8 min) to give desired 4-chloro-3-(7, 7-difluoro-4-azaspiro [2.5]octan-4-yl)-6-fluoro-1-(1-isopropylsulfonylpyrrol-3-yl) sulfonyl-indazole (10 mg, 18.0 μmol, 19.0% yield, 99.38% purity) as a white solid. ¹H NMR (400 MHz, DMSO-d₆) δ 7.99-8.12 (m, 1H), 7.81 (dd, J=8.94, 2.06 Hz, 1H), 7.53 (dd, J=9.13, 2.00 Hz, 1H), 7.37 (dd, J=3.25, 2.38 Hz, 1H), 6.57 (dd, J=3.38, 1.63 Hz, 1H), 3.87-4.00 (m, 1H), 3.56 (br s, 2H), 2.10-2.44 (m, 2H), 1.81-1.97 (m, 2H), 1.09 (d, J=6.75 Hz, 6H), 0.72 (s, 2H), 0.47 (br s, 2H). HPLC: 99.38% (220 nm), 99.36% (215 nm), 99.14% (254 nm). MS (ESI): mass calcd. For C₂₁H₂₂ClF₃N₄O₄S₂ 550.1 m/z found 551.0 [M+H]⁺.

### Compound 192: 4-chloro-6-fluoro-3-(7-fluoro-4-azaspiro [2.5]octan-4-yl)-1-(1-isopropylsulfonylpyrrol-3-yl) sulfonyl-indazole

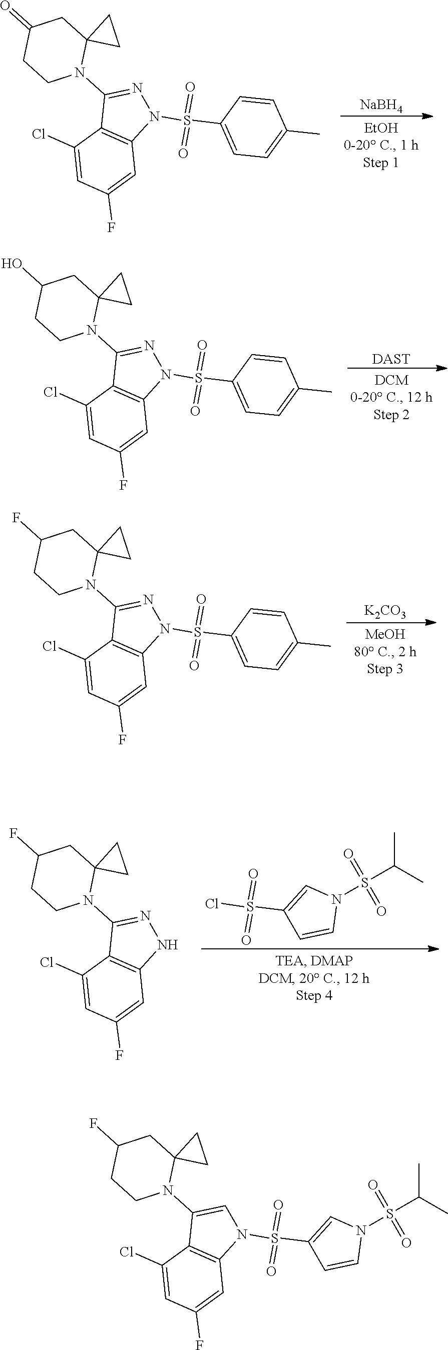

Step 1: To a solution of 4-[4-chloro-6-fluoro-1-(p-tolylsulfonyl) indazol-3-yl]-4-azaspiro [2.5]octan-7-one (100 mg, 223 umol, 1 eq) in EtOH (3 mL) was added NaBH4 (16.9 mg, 447 μmol, 2 eq) at 0° C. The mixture was stirred at 20° C. for 1 hour. LC-MS showed 4-[4-chloro-6-fluoro-1-(p-tolylsulfonyl) indazol-3-yl]-4-azaspiro [2.5]octan-7-one was consumed completely and one main peak with desired mass was detected. The reaction was quenched with NH₄Cl solution (5 mL) and stirred for 15 minutes. The mixture was concentrated to get a residue. The residue was added water (5 mL) and extracted with EtOAc (20 mL). The combined organics were dried over anhydrous sodium sulfate and concentrated to get a residue. The residue was purified by prep-TLC (SiO₂, petroleum ether/EtOAc=1/1) to give desired 4-[4-chloro-6-fluoro-1-(p-tolylsulfonyl) indazol-3-yl]-4-azaspiro [2.5]octan-7-ol (80 mg, 178 μmol, 79.6% yield) as a colourless oil. MS (ESI): mass calcd. For C₂₁H₂₁ClFN₃O₃S 449.1 m/z found 450.1 [M+H]⁺.

Step 2: To a solution of 4-[4-chloro-6-fluoro-1-(p-tolylsulfonyl) indazol-3-yl]-4-azaspiro [2.5]octan-7-ol (80 mg, 178 μmol, 1 eq) in DCM (3 mL) was added DAST (287 mg, 1.78 mmol, 235 μL, 10 eq) dropwise at 0° C. The mixture was stirred at 20° C. for 12 hours. LC-MS showed 4-[4-chloro-6-fluoro-1-(p-tolylsulfonyl) indazol-3-yl]-4-azaspiro [2.5]octan-7-ol was consumed completely and one main peak with desired mass was detected. The reaction mixture was quenched by addition MeOH (5 mL) at 0° C., and then concentrated under reduced pressure to give desired 4-chloro-6-fluoro-3-(7-fluoro-4-azaspiro [2.5]octan-4-yl)-1-(p-tolylsulfonyl) indazole (80 mg, crude) as a yellow oil. MS (ESI): mass calcd. For C₂₁H₂₀ClF₂N₃O₂S 451.1 m/z found 452.1 [M+H]⁺.

Step 3: To a solution of 4-chloro-6-fluoro-3-(7-fluoro-4-azaspiro [2.5]octan-4-yl)-1-(p-tolylsulfonyl) indazole (80 mg, 177 μmol, 1 eq) in MeOH (3 mL) was added K₂CO₃ (122 mg, 885 μmol, 5 eq). The mixture was stirred at 80° C. for 2 hours. LC-MS showed 4-chloro-6-fluoro-3-(7-fluoro-4-azaspiro [2.5]octan-4-yl)-1-(p-tolylsulfonyl) indazole was consumed completely and one main peak with desired mass was detected. The reaction mixture was concentrated under reduced pressure to remove MeOH. The residue was diluted with H₂O (5 mL) and extracted with EtOAc (30 mL). The combined organic layers were washed with brine (20 mL), dried over anhydrous Na₂SO₄, filtered and concentrated under reduced pressure to give a residue. The residue was purified by prep-TLC (SiO₂, petroleum ether/EtOAc=2/1) to give desired 4-chloro-6-fluoro-3-(7-fluoro-4-azaspiro [2.5]octan-4-yl)-1H-indazole (17 mg, 57.1 μmol, 32.3% yield) as a yellow oil. MS (ESI): mass calcd. For C₁₄H₁₄ClF₂N₃ 297.1 m/z found 298.1 [M+H]⁺.

Step 4: To a solution of 4-chloro-6-fluoro-3-(7-fluoro-4-azaspiro [2.5]octan-4-yl)-1H-indazole (17 mg, 57.1 μmol, 1 eq) and 1-isopropylsulfonylpyrrole-3-sulfonyl chloride (31.0 mg, 114 μmol, 2 eq) in DCM (1 mL) was added TEA (17.3 mg, 171 μmol, 23.8 μL, 3 eq) and DMAP (698 g, 5.71 μmol, 0.1 eq). The mixture was stirred at 20° C. for 12 hours. LC-MS showed 4-chloro-6-fluoro-3-(7-fluoro-4-azaspiro [2.5]octan-4-yl)-1H-indazole was consumed completely and one main peak with desired mass was detected. The reaction mixture was diluted with H₂O (5 mL) and extracted with DCM (10 mL). The combined organic layers were washed with brine (10 mL), dried over anhydrous Na₂SO₄, filtered and concentrated under reduced pressure to give a residue. The residue was purified by prep-HPLC (neutral condition: column: Waters Xbridge BEH C18 100*30 mm*10 μm; mobile phase: [water (NH₄HCO₃)-ACN]; B %: 55%-85%, 8 min) to give desired 4-chloro-6-fluoro-3-(7-fluoro-4-azaspiro [2.5]octan-4-yl)-1-(1-isopropylsulfonylpyrrol-3-yl)sulfonyl-indazole (10 mg, 18.4 μmol, 32.3% yield, 98.29% purity) as a white solid. ¹H NMR (400 MHz, DMSO-d₆) δ 8.02 (t, J=1.94 Hz, 1H), 7.79 (dd, J=8.88, 2.13 Hz, 1H), 7.51 (dd, J=9.13, 2.13 Hz, 1H), 7.36 (dd, J=3.25, 2.50 Hz, 1H), 6.55 (dd, J=3.38, 1.63 Hz, 1H), 4.81-5.08 (m, 1H), 3.94 (quin, J=6.75 Hz, 1H), 3.55-3.67 (m, 1H), 3.37 (br s, 1H), 1.94-2.24 (m, 2H), 1.72-1.91 (m, 1 H), 1.58 (br d, J=8.25 Hz, 1H), 1.08 (dd, J=6.75, 1.88 Hz, 6H), 0.56-0.75 (m, 2H), 0.32-0.49 (m, 2H). HPLC: 98.29% (220 nm), 96.94% (215 nm), 100.0% (254 nm). MS (ESI): mass calcd. For C₂₁H₂₃ClF₂N₄O₄S₂ 532.1 m/z found 533.1 [M+H]⁺.

### Compound 193: 4-[3-[7-(5-chloro-3-fluoro-2-pyridyl)-4, 7-diazaspiro [2.5]octan-4-yl]-4, 7-difluoro-indazol-1-yl]sulfonyl-2-isopropylsulfonyl-thiazole

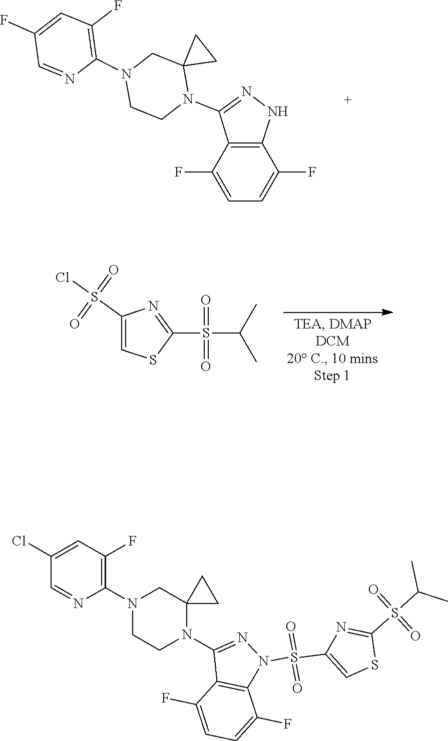 [SMILES: Fc1cnc(N2CCN(c3n[nH]c4c(F)ccc(F)c34)C3(CC3)C2)c(F)c1 ; CC(C)S(=O)(=O)c1nc(S(=O)(=O)Cl)cs1 ; CC(C)S(=O)(=O)c1nc(S(=O)(=O)n2nc(N3CCN(c4ncc(Cl)cc4F)CC34CC4)c3c(F)ccc(F)c32)cs1]

Step 1: To a solution of 3-[7-(5-chloro-3-fluoro-2-pyridyl)-4, 7-diazaspiro [2.5]octan-4-yl]-4, 7-difluoro-1H-indazole (25 mg, 63.5 μmol, 1 eq) and 2-isopropylsulfonylthiazole-4-sulfonyl chloride (27.6 mg, 95.2 μmol, 1.5 eq) in DCM (1 mL) was added TEA (19.3 mg, 190 μmol, 26.5 μL, 3 eq) and DMAP (776 μg, 6.35 μmol, 0.1 eq), and then the mixture was stirred at 20° C. for 10 mins. LC-MS showed 4-[3-[7-(5-chloro-3-fluoro-2-pyridyl)-4, 7-diazaspiro [2.5]octan-4-yl]-4, 7-difluoro-indazol-1-yl]sulfonyl-2-isopropylsulfonyl-thiazole was consumed completely and one main peak with desired mass was detected. The residue was diluted with H₂O 10 mL and extracted with EtOAc (5 mL*3). The combined organic layers were washed with brine (10 mL*1), dried over Na₂SO₄, filtered and concentrated under reduced pressure to give a residue. The residue was purified by prep-TLC (SiO₂, petroleum ether/EtOAc=3/1) to give desired 4-[3-[7-(5-chloro-3-fluoro-2-pyridyl)-4, 7-diazaspiro [2.5]octan-4-yl]-4, 7-difluoro-indazol-1-yl]sulfonyl-2-isopropylsulfonyl-thiazole (8.4 mg, 12.5 μmol, 19.7% yield, 96.14% purity) as a white solid. ¹H NMR (400 MHz, DMSO-d₆) δ 9.21 (s, 1H), 8.09-8.05 (m, 1H), 7.87-7.81 (m, 1H), 7.65-7.57 (m, 1H), 7.34-7.27 (m, 1H), 3.70-3.63 (m, 2H), 3.53-3.39 (m, 3H), 3.33-3.19 (m, 2H), 1.04-0.90 (m, 6H), 0.84-0.74 (m, 2H), 0.50-0.39 (m, 2H). HPLC: 96.14% (220 nm), 96.06% (215 nm), 97.51% (254 nm). MS (ESI): mass calcd. For C₂₄H₂₂ClF₃N₆O₄S₃ 646.08, m/z found 647.0 [M+H]⁺.

### Compound 194: 4-[3-[7-(3, 5-difluoro-2-pyridyl)-4, 7-diazaspiro [2.5]octan-4-yl]-4, 7-difluoro-indazol-1-yl]sulfonyl-2-isopropylsulfonyl-thiazole

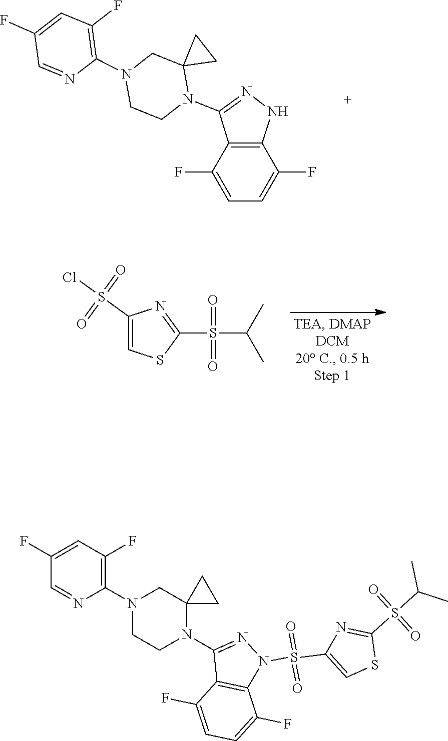 [SMILES: Fc1cnc(N2CCN(c3n[nH]c4c(F)ccc(F)c34)C3(CC3)C2)c(F)c1 ; CC(C)S(=O)(=O)c1nc(S(=O)(=O)Cl)cs1 ; CC(C)S(=O)(=O)c1nc(S(=O)(=O)n2nc(N3CCN(c4ncc(F)cc4F)CC34CC4)c3c(F)ccc(F)c32)cs1]

Step 1: To a solution of 3-[7-(3, 5-difluoro-2-pyridyl)-4, 7-diazaspiro [2.5]octan-4-yl]-4, 7-difluoro-1H-indazole (30 mg, 79.5 μmol, 1 eq) and 2-isopropylsulfonylthiazole-4-sulfonyl chloride (34.6 mg, 119 μmol, 1.5 eq) in DCM (1 mL) was added TEA (24.1 mg, 239 μmol, 33.2 μL, 3 eq) and DMAP (971 μg, 7.95 μmol, 0.1 eq), and then the mixture was stirred at 20° C. for 0.5 hour. LC-MS showed 3-[7-(3, 5-difluoro-2-pyridyl)-4, 7-diazaspiro [2.5]octan-4-yl]-4, 7-difluoro-1H-indazole was consumed completely and one main peak with desired mass was detected. The residue was diluted with H₂O (10 mL) and extracted with EtOAc (5 mL*3). The combined organic layers were washed with brine (10 mL*1), dried over Na₂SO₄, filtered and concentrated under reduced pressure to give a residue. The residue was purified by prep-TLC (SiO₂, petroleum ether/EtOAc=3/1) to give desired 4-[3-[7-(3, 5-difluoro-2-pyridyl)-4, 7-diazaspiro [2.5]octan-4-yl]-4, 7-difluoro-indazol-1-yl]sulfonyl-2-isopropylsulfonyl-thiazole (11 mg, 16.8 μmol, 21.1% yield, 96.13% purity) as a white solid. ¹H NMR (400 MHz, DMSO-d₆) δ 9.22-9.19 (m, 1H), 8.11-8.07 (m, 1H), 7.84-7.75 (m, 1H), 7.64-7.57 (m, 1H), 7.34-7.26 (m, 1H), 3.71-3.63 (m, 2H), 3.51-3.34 (m, 3H), 3.23-3.10 (m, 2H), 1.01-0.94 (m, 6H), 0.82-0.76 (m, 2H), 0.49-0.41 (in, 2H). HPLC: 96.13% (220 nm), 96.22% (215 nm), 98.85% (254 nm). MS (ESI): mass calcd. For C₂₄H₁₂₂F₄N₆O₄S₃ 630.08, m/z found 631.0 [M+H].

### Compound 195: 4-((4, 7-difluoro-3-(7-(5-fluoropyrimidin-2-yl)-4, 7-diazaspiro 12.51 octan-4-yl)-1H-indazol-1-yl) sulfonyl)-2-(isopropylsulfonyl) thiazole

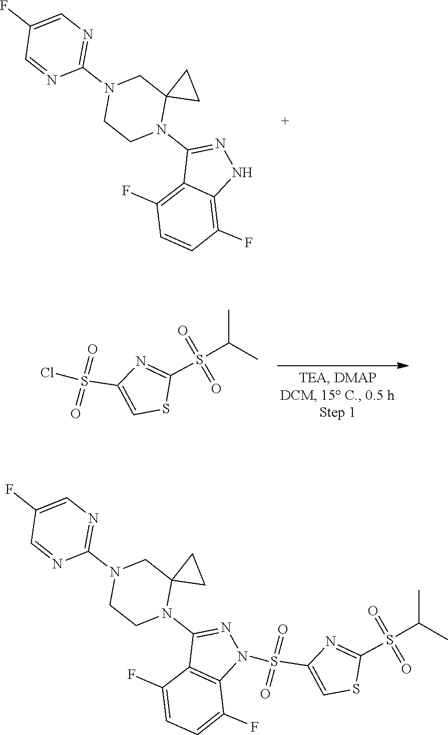 [SMILES: Fc1cnc(N2CCN(c3n[nH]c4c(F)ccc(F)c34)C3(CC3)C2)nc1 ; CC(C)S(=O)(=O)c1nc(S(=O)(=O)Cl)cs1 ; CC(C)S(=O)(=O)c1nc(S(=O)(=O)n2nc(N3CCN(c4ncc(F)cn4)CC34CC4)c3c(F)ccc(F)c32)cs1]

Step 1: To a solution of 4,7-difluoro-3-[7-(5-fluoropyrimidin-2-yl)-4,7-diazaspiro[2.5]octan-4-yl]-1H-indazole (50 mg, 139 μmol, 1 eq) and 2-isopropylsulfonylthiazole-4-sulfonyl chloride (60.3 mg, 208 μmol, 1.5 eq) in DCM (1 mL) was added TEA (28.1 mg, 278 μmol, 38.6 μL, 2 eq) and DMAP (1.70 mg, 13.9 μmol, 0.1 eq). The mixture was stirred at 15° C. for 0.5 hour. LC-MS showed 4, 7-difluoro-3-[7-(5-fluoropyrimidin-2-yl)-4, 7-diazaspiro [2.5]octan-4-yl]-1H-indazole was consumed completely and desired mass was detected. The crude was added H₂O (10 mL), and extracted with EtOAc (15 mL*3). The combined organic layers were washed with brine 10 mL, dried over Na₂SO₄, filtered and concentrated under reduced pressure to give a residue. The residue was purified by prep-HPLC (neutral condition, column: Phenomenex C18 75*30 mm*3 μm; mobile phase: [water(NH₄HCO₃)-ACN]; B %: 25%-60%, 8 min) to give desired 4-((4, 7-difluoro-3-(7-(5-fluoropyrimidin-2-yl)-4, 7-diazaspiro [2.5]octan-4-yl)-1H-indazol-1-yl) sulfonyl)-2-(isopropylsulfonyl) thiazole (13.7 mg, 22.2 μmol, 16.0% yield, 99.22% purity) as a white solid. ¹H NMR (400 MHz, DMSO-d₆) δ 9.21 (s, 1H), 8.46 (d, J=0.6 Hz, 2H), 7.67-7.57 (m, 1H), 7.36-7.26 (m, 1H), 3.81 (s, 2H), 3.60 (br s, 5H), 0.98 (d, J=6.9 Hz, 6H), 0.83-0.77 (m, 2H), 0.44 (br s, 2H). HPLC: 99.22% (220 nm), 99.27% (215 nm), 99.32% (254 nm). MS (ESI): mass calcd. For C₂₃H₂₂F₃N₇O₄S₃ 613.08 m/z found 614.2 [M+H]⁺.

### Compound 196: 4-((4-chloro-3-(7-(5-chloro-3-fluoropyridin-2-yl)-4, 7-diazaspiro [2.5]octan-4-yl)-1H-indazol-1-yl) sulfonyl)-2-(isopropylsulfonyl) thiazole

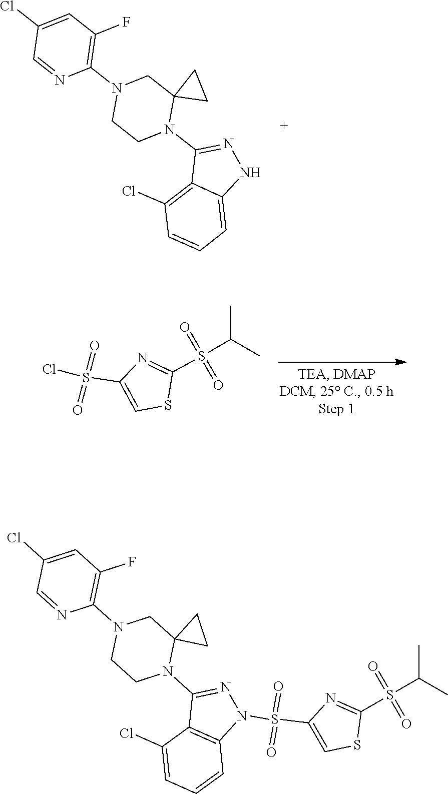 [SMILES: Fc1cc(Cl)cnc1N1CCN(c2n[nH]c3cccc(Cl)c23)C2(CC2)C1 ; CC(C)S(=O)(=O)c1nc(S(=O)(=O)Cl)cs1 ; CC(C)S(=O)(=O)c1nc(S(=O)(=O)n2nc(N3CCN(c4ncc(Cl)cc4F)CC34CC4)c3c(Cl)cccc32)cs1]

Step 1: To a solution of 1-cyclopropylsulfonylpyrrole-3-sulfonyl chloride (100 mg, 371 μmol, 1 eq) and 3-chloro-2-(4,4-dimethyl-1-piperidyl) aniline (88.5 mg, 371 μmol, 1 eq) in DCM (2 mL) was added TEA (93.8 mg, 927 μmol, 129 μL, 2.5 eq) and DMAP (4.53 mg, 37.1 μmol, 0.1 eq). The mixture was stirred at 25° C. for 30 mins. LC-MS showed 1-cyclopropylsulfonylpyrrole-3-sulfonyl chloride remained and desired mass was detected. The residue was diluted with water (20 mL) and extracted with DCM (20 mL). The combined organic layers were washed with brine (20 mL), dried over Na₂SO₄, filtered and concentrated under reduced pressure to give a residue. The residue was purified by prep-TLC (SiO₂, petroleum ether/EtOAc=3/1) to give desired N-[3-chloro-2-(4, 4-dimethyl-1-piperidyl) phenyl]-1-cyclopropylsulfonyl-pyrrole-3-sulfonamide (39.1 mg, 82.0 μmol, 22.1% yield, 99.04% purity) as a white solid. ¹H NMR (400 MHz, DMSO-d₆) δ 8.80 (s, 1H), 7.73 (t, J=1.9 Hz, 1H), 7.42-7.35 (m, 2H), 7.22-7.13 (m, 2H), 6.50 (dd, J=1.6, 3.3 Hz, 1H), 3.44-3.34 (m, 2H), 3.25-3.17 (m, 1H), 2.33-2.18 (m, 2H), 1.51 (br s, 2H), 1.36 (br s, 2H), 1.27-1.22 (m, 2H), 1.17-1.10 (m, 2H), 0.98 (br s, 6H). HPLC: 99.04% (220 nm), 99.06% (215 nm), 98.83% (254 nm). MS (ESI): mass calcd. For C₂₀H₂₆ClN₃O₄S₂ 471.11 m/z found 472.1 [M+H]⁺.

### Compound 197: 4-[4-chloro-3-[7-(3, 5-difluoro-2-pyridyl)-4, 7-diazaspiro [2.5]octan-4-yl]-6-fluoro-indazol-1-yl]sulfonyl-2-isopropylsulfonyl-thiazole

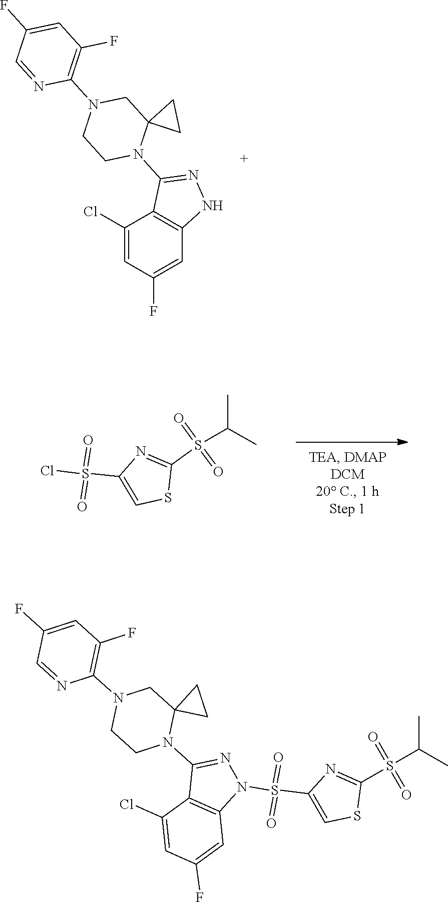 [SMILES: Fc1cnc(N2CCN(c3n[nH]c4cc(F)cc(Cl)c34)C3(CC3)C2)c(F)c1 ; CC(C)S(=O)(=O)c1nc(S(=O)(=O)Cl)cs1 ; CC(C)S(=O)(=O)c1nc(S(=O)(=O)n2nc(N3CCN(c4ncc(F)cc4F)CC34CC4)c3c(Cl)cc(F)cc32)cs1]

Step 1: To a solution of 4-chloro-3-[7-(3, 5-difluoro-2-pyridyl)-4, 7-diazaspiro [2.5]octan-4-yl]-6-fluoro-1H-indazole (30 mg, 76.2 μmol, 1 eq) and 2-isopropylsulfonylthiazole-4-sulfonyl chloride (33.1 mg, 114 μmol, 1.5 eq) in DCM (1 mL) was added TEA (7.71 mg, 76.2 μmol, 10.6 μL, 1 eq) and DMAP (931 μg, 7.62 μmol, 0.1 eq). The mixture was stirred at 20° C. for 1 hour. LC-MS showed 4-chloro-3-[7-(3, 5-difluoro-2-pyridyl)-4, 7-diazaspiro [2.5]octan-4-yl]-6-fluoro-1H-indazole was consumed completely and one main peak with desired mass was detected. The reaction mixture was diluted with H₂O (5 mL) and extracted with DCM (8 mL). The combined organic layers were washed with brine (8 mL), dried over anhydrous Na₂SO₄, filtered and concentrated under reduced pressure to give a residue. The residue was purified by prep-HPLC (neutral condition: column: Waters Xbridge BEH C18 100*30 mm*10 um; mobile phase: [water (NH₄HCO₃)-ACN]; B %: 60%-90%, 8 min) to give desired 4-[4-chloro-3-[7-(3, 5-difluoro-2-pyridyl)-4, 7-diazaspiro [2.5]octan-4-yl]-6-fluoro-indazol-1-yl]sulfonyl-2-isopropylsulfonyl-thiazole (10.2 mg, 15.7 μmol, 20.5% yield, 99.27% purity) as a white solid. ¹H NMR (400 MHz, DMSO-d₆) δ 9.28 (s, 1H) 8.09 (d, J=2.38 Hz, 1H) 7.75-7.84 (m, 2H) 7.60 (dd, J=9.07, 1.69 Hz, 1H) 3.59 (brs, 2H) 3.49-3.55 (m, 1H) 3.47 (brd, J=11.26 Hz, 2H) 3.26 (brs, 2H) 0.99 (d, J=6.88 Hz, 6H) 0.72 (br s, 2H) 0.39 (br s, 2H). HPLC: 99.27% (220 nm), 99.43% (215 nm), 99.92% (254 nm). MS (ESI): mass calcd. For C₂₄H₂₂ClF₃N₆O₄S₃ 646.05 m/z found 646.9 [M+H]⁺.

### Compound 198: 4-((3-(7-(3, 5-difluoropyridin-2-yl)-4, 7-diazaspiro [2.5]octan-4-yl)-4, 6-difluoro-1H-indazol-1-yl) sulfonyl)-2-(isopropylsulfonyl) thiazole

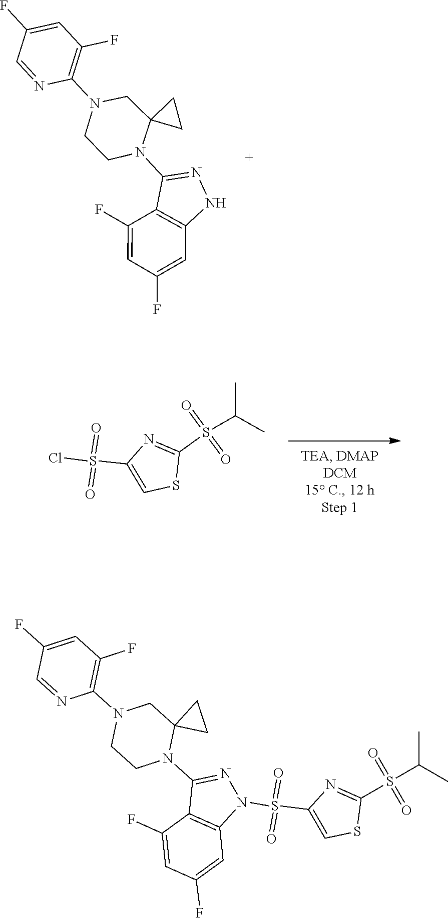 [SMILES: Fc1cnc(N2CCN(c3n[nH]c4cc(F)cc(F)c34)C3(CC3)C2)c(F)c1 ; CC(C)S(=O)(=O)c1nc(S(=O)(=O)Cl)cs1 ; CC(C)S(=O)(=O)c1nc(S(=O)(=O)n2nc(N3CCN(c4ncc(F)cc4F)CC34CC4)c3c(F)cc(F)cc32)cs1]

Step 1: To a solution of 3-[7-(3, 5-difluoro-2-pyridyl)-4, 7-diazaspiro [2.5]octan-4-yl]-4, 6-difluoro-1H-indazole (30 mg, 79.5 μmol, 1 eq) and 2-isopropylsulfonylthiazole-4-sulfonyl chloride (34.5 mg, 119 μmol, 1.5 eq) in DCM (1 mL) was added TEA (16.1 mg, 159 μmol, 22.1 μL, 2 eq) and DMAP (971 μg, 7.95 μmol, 0.1 eq). The mixture was stirred at 15° C. for 12 hours. LC-MS showed 3-[7-(3, 5-difluoro-2-pyridyl)-4, 7-diazaspiro [2.5]octan-4-yl]-4, 6-difluoro-1H-indazole was consumed completely and desired mass was detected. The reaction mixture was concentrated under reduced pressure to give a residue. The crude product was purified by reversed-phase HPLC (column: C18 20-35 um 100 A 40 g; mobile phase: [water-ACN]; B %: 0%-25% @50 mL/min) to give desired 4-((3-(7-(3, 5-difluoropyridin-2-yl)-4, 7-diazaspiro [2.5]octan-4-yl)-4, 6-difluoro-1H-indazol-1-yl) sulfonyl)-2-(isopropylsulfonyl) thiazole (8.6 mg, 13.0 μmol, 16.3% yield, 94.97% purity) as a white solid. ¹H NMR (400 MHz, DMSO-d₆) δ 9.23 (s, 1H), 8.05 (d, J=2.4 Hz, 1H), 7.81-7.72 (m, 1H), 7.67-7.61 (m, 1H), 7.43-7.34 (m, 1H), 3.70-3.64 (m, 2H), 3.51-3.45 (m, 1H), 3.40-3.31 (m, 2H), 3.22 (br s, 2H), 0.95 (d, J=6.8 Hz, 6H), 0.78-0.73 (m, 2H), 0.45 (br s, 2H). HPLC: 91.40% (220 nm), 92.08% (215 nm), 94.97 (254 nm). MS (ESI): mass calcd. For C₂₄H₂₂F₄N₆O₄S₃ 630.08 m/z found 631.2 [M+H]⁺.

### Compound 199: 4-chloro-3-[7-(3, 5-difluoro-2-pyridyl)-4, 7-diazaspiro [2.5]octan-4-yl]-6-fluoro-1-(1-isopropylsulfonylpyrazol-3-yl) sulfonyl-indazole

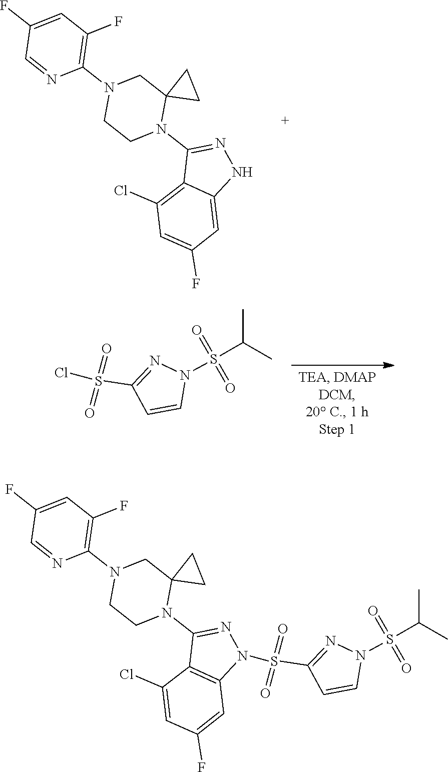 [SMILES: Fc1cnc(N2CCN(c3n[nH]c4cc(F)cc(Cl)c34)C3(CC3)C2)c(F)c1 ; CC(C)S(=O)(=O)n1ccc(S(=O)(=O)Cl)n1 ; CC(C)S(=O)(=O)n1ccc(S(=O)(=O)n2nc(N3CCN(c4ncc(F)cc4F)CC34CC4)c3c(Cl)cc(F)cc32)n1]

Step 1: To a solution of 4-chloro-3-[7-(3, 5-difluoro-2-pyridyl)-4, 7-diazaspiro [2.5]octan-4-yl]-6-fluoro-1H-indazole (50 mg, 127 μmol, 1 eq) and 1-isopropylsulfonylpyrazole-3-sulfonyl chloride (41.6 mg, 152 μmol, 1.2 eq) in DCM (1 mL) was added TEA (38.5 mg, 381 μmol, 53.0 μL, 3 eq) and DMAP (1.55 mg, 12.7 μmol, 0.1 eq). The mixture was stirred at 20° C. for 1 hour. LC-MS showed 4-chloro-3-[7-(3, 5-difluoro-2-pyridyl)-4, 7-diazaspiro [2.5]octan-4-yl]-6-fluoro-1H-indazole was consumed completely and one main peak with desired mass was detected. The reaction mixture was diluted with H₂O (5 mL) and extracted with DCM (10 mL). The combined organic layers were washed with brine (8 mL), dried over anhydrous Na₂SO₄, filtered and concentrated under reduced pressure to give a residue. The residue was purified by prep-HPLC (neutral condition: column: Phenomenex C18 75*30 mm*3 um; mobile phase: [water (NH₄HCO₃)-ACN]; B %: 65%-85%, 8 min) to give desired 4-chloro-3-[7-(3, 5-difluoro-2-pyridyl)-4, 7-diazaspiro [2.5]octan-4-yl]-6-fluoro-1-(1-isopropylsulfonylpyrazol-3-yl)sulfonyl-indazole (3.0 mg, 4.75 μmol, 3.74% yield, 99.75% purity) as a white solid. ¹H NMR (400 MHz, DMSO-d₆) δ 8.54 (d, J=2.75 Hz, 1H) 8.10 (d, J=2.50 Hz, 1H) 7.75-7.85 (m, 2H) 7.60 (dd, J=9.07, 1.94 Hz, 1H) 7.17 (d, J=2.75 Hz, 1H) 3.89 (dt, J=13.51, 6.75 Hz, 1H) 3.61 (br t, J=4.44 Hz, 2H) 3.49 (br s, 2H) 3.25-3.31 (m, 2H) 0.98 (d, J=6.75 Hz, 6H) 0.76 (br s, 2H) 0.48 (br s, 2H). HPLC: 99.75% (220 nm), 99.19% (215 nm), 100.00% (254 nm). MS (ESI): mass calcd. For C₂₄H₂₃ClF₃N₇O₄S₂ 629.09 m/z found 630.1 [M+H]⁺.

### Compound 200: 3-(7-(3, 5-difluoropyridin-2-yl)-4, 7-diazaspiro [2.5]octan-4-yl)-4, 6-difluoro-1-((1-(isopropylsulfonyl)-1H−1, 2, 4-triazol-3-yl) sulfonyl)-1H-indazole

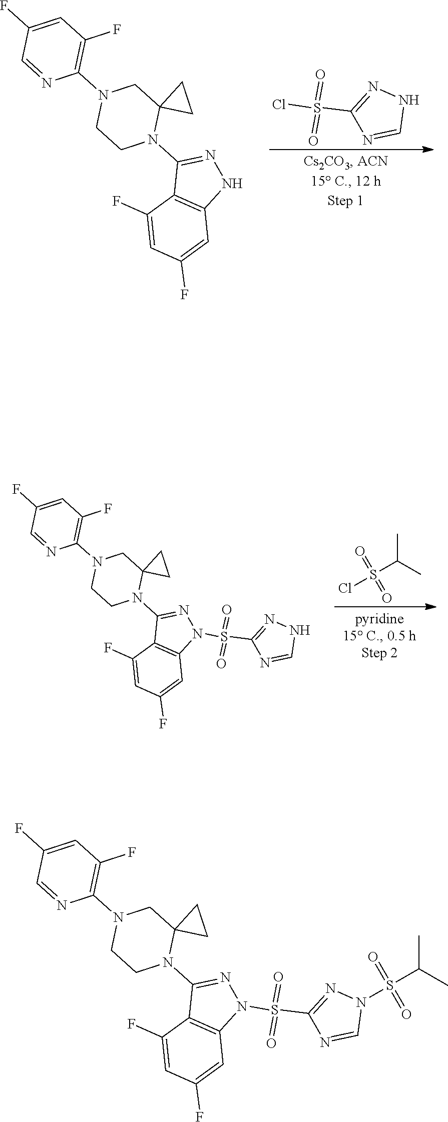

Step 1: To a solution of 3-[7-(3,5-difluoro-2-pyridyl)-4,7-diazaspiro[2.5]octan-4-yl]-4,6-difluoro-1H-indazole (50 mg, 133 μmol, 1 eq) and 1H−1,2,4-triazole-3-sulfonyl chloride (22.2 mg, 133 μmol, 1 eq) in ACN (1 mL) was added Cs₂CO₃ (216 mg, 663 μmol, 5 eq). The mixture was stirred at 15° C. for 12 hours. LC-MS showed 3-[7-(3, 5-difluoro-2-pyridyl)-4, 7-diazaspiro [2.5]octan-4-yl]-4, 6-difluoro-1H-indazole was consumed completely and desired mass was detected. The crude was added H₂O (10 mL), and extracted with EtOAc (45 mL). The combined organic layers were washed with brine (10 mL), dried over Na₂SO₄, filtered and concentrated under reduced pressure to give a residue 1-((1H−1, 2, 4-triazol-3-yl) sulfonyl)-3-(7-(3, 5-difluoropyridin-2-yl)-4, 7-diazaspiro [2.5]octan-4-yl)-4, 6-difluoro-1H-indazole (50 mg, crude) as a white solid. MS (ESI): mass calcd. For C₂₀H₁₆F₄N₈O₂S 508.11, m/z found 509.2 [M+H]⁺.

Step 2: To a solution of 3-[7-(3,5-difluoro-2-pyridyl)-4,7-diazaspiro[2.5]octan-4-yl]-4,6-difluoro-1-(1H−1,2,4-triazol-3-ylsulfonyl) indazole (50 mg, 98.3 μmol, 1 eq) in Py (1 mL) was added propane-2-sulfonyl chloride (70.1 mg, 492 μmol, 54.8 μL, 5 eq). The mixture was stirred at 15° C. for 0.5 hour. LC-MS showed 3-[7-(3, 5-difluoro-2-pyridyl)-4, 7-diazaspiro [2.5]octan-4-yl]-4, 6-difluoro-1-(1H−1, 2, 4-triazol-3-ylsulfonyl) indazole was consumed completely and desired mass was detected. The reaction mixture was concentrated under reduced pressure to give a residue. The residue was purified by prep-HPLC (neutral condition, column: column: Phenomenex Luna C18 75*30 mm*3 um; mobile phase: [water(FA)-ACN]; B %: 50%-80%, 8 min) give desired 3-(7-(3, 5-difluoropyridin-2-yl)-4, 7-diazaspiro [2.5]octan-4-yl)-4, 6-difluoro-1-((1-(isopropylsulfonyl)-1H−1, 2, 4-triazol-3-yl) sulfonyl)-1H-indazole (2.0 mg, 3.01 μmol, 3.06% yield, 92.37% purity) as a white solid. ¹H NMR (400 MHz, DMSO-d₆) δ 9.39 (s, 1H), 8.05-8.03 (m, 1H), 7.80-7.73 (m, 1H), 7.59 (dd, J=1.8, 8.8 Hz, 1H), 7.45-7.37 (m, 1H), 4.14-4.06 (m, 1H), 3.71-3.67 (m, 2H), 3.30-3.12 (m, 4H), 1.14 (d, J=6.8 Hz, 6H), 0.77-0.73 (m, 2H), 0.56-0.51 (m, 2H). HPLC: 88.18% (220 nm), 82.81% (215 nm), 92.37% (254 nm). MS (ESI): mass calcd. For C₂₃H₂₂F₄N₈O₄S₂614.11 m/z found 615.2 [M+H]⁺.

### Biological Assays

### Lysosomal Ca²⁺ Imaging

GCaMP3-ML1 expression was induced in Tet-On HEK-GCaMP3-ML1 cells 20-24 h prior to experiments using 0.01 μg/mL doxycycline. GCaMP3-ML1 fluorescence was monitored at an excitation wavelength of 470 nm (F₄₇₀) using an EasyRatio Pro system (PTI). Cells were bathed in Tyrode's solution containing 145 mM NaCl, 5 mM KCl, 2 mM CaCl₂, 1 mM MgCl₂, 10 mM Glucose, and 20 mM Hepes (pH 7.4). Lysosomal Ca²⁺ release was measured in a zero Ca²⁺ solution containing 145 mM NaCl, 5 mM KCl, 3 nM MgCl₂, 10 mM glucose, 1 mM EGTA, and 20 mM HEPES (pH 7.4). Ca²⁺ concentration in the nominally free Ca²⁺ solution is estimated to be 1-10 μM. With 1 mM EGTA, the free Ca²⁺ concentration is estimated to be <10 nM based on the Maxchelator software (http://maxchelator.stanford.edu/). Experiments were carried out 0.5 to 6 hrs after plating.

The EC₅₀ data for some of compounds are less than 10 uM.

### TFEB Nuclear Translocation Assay

TFEB is a transcription factor and master regulator of lysosome biogenesis and autophagy. TFEB activation is shown to induce cellular clearance in a variety of LSDs and common neurodegenerative diseases. Hence TFEB activity can be used to evaluate the cellular efficacy of TRPML agonists.

Intracellular TFEB localization is determined either by immunofluorescence in Hela cells or by fluorescent microscopy in Hela cells stably expressing TFEB-GFP. Detailed procedures are as follows.

Cells grown on cover slips were treated with TRPML1 agonist(s) or antagonist(s) for indicated time period and then fixed with 4% paraformaldehyde for 15 minutes at room temperature. For immunofluorescent detection of endogenous TFEB, cells were permeabilized with 0.3% Triton X-100, blocked with 1% bovine serum albumin (BSA) in phosphate buffered saline (PBS) and then immunostained with anti-TFEB antibody (1:200; Cell Signaling Technology) at 4° C. for overnight. The stained cells were then incubated with secondary antibodies conjugated to Alexa Fluor 568 or 488 (ThermoFisher) for 1 h, then 4′,6-diamidino-2-phenylindole (DAPI) for 15 min (to stain the cell nucleus) and mounted on glass slides with Fluoromount-G (Southern Biotech) for observation. Images were acquired with a Spinning-Disk Confocal microscope (Olympus) and Metamorph software (Molecular Devices).

Average ratios of nuclear versus cytosolic TFEB fluorescence intensity (>50 randomly-selected cells per experiment) were determined with ImageJ software (NIH). Exemplary data of TFEB nuclear translocation assay are provided in Table 1.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound having the structure of formula I: 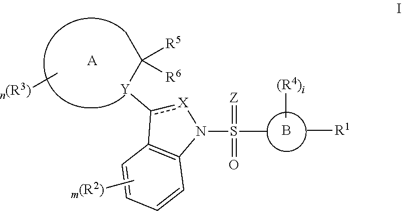
or a pharmaceutically acceptable form or an isotope derivative thereof, wherein
Ring A is a 4-, 5- or 6-membered substituted heterocycle;
Ring B a substituted or unsubstituted, 6-membered aryl or heteroaryl ring, 5-membered heteroaryl ring, or a substituted or unsubstituted bi- or multi-cyclic carbocyclic or heterocyclic ring;
X is CH or N;
Y is CH or N;
Z is O or NH;
R¹ is selected from the group consisting of C₁₋₅ alkyl, C₃₋₇ cycloalkyl, heterocyclic, halo, OR, SR, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, wherein the alkyl, cycloalkyl, heterocyclic, R and R′ is independently substituted with 0-6F's;
each R² is independently selected from the group consisting of C₁₋₆ alkyl, halo, oxo, OH, CN, OR, NRR′, N(R)C(═O) RR′, N(R)C(═O)(O)RR′, OC(═O)NRR′, C(═O)R, C(═O)NRR′, N(R)S(O)₂RR′, S(O)₂R, or S(O)₂NRR′;
each R³ is independently selected from the group consisting of C₁₋₅ alkyl, C₃₋₇ cycloalkyl, heterocycle, heteroaryl, O, halo, CF₃, CH₂CF₃, CN, N(R)C(═O)R′, N(R)C(═O)(O)R′, OC(═O)NRR′, C(═O)R, C(═O)NRR′, N(R)S(O)₂R′, S(O)R, S(O)NHR, S(O)₂R, and S(O)₂NRR′, which where applicable is optionally substituted with 1-3 groups selected from halo, CF₃, CH₂CF₃, CN, OH, C₁₋₅ alkyl, and C₃₋₇ cycloalkyl; two R³'s attached to the same carbon atom together form a C═O; or two R³'s, together with the carbon and/or nitrogen atom(s) to which they are attached, form a 3- to 6-membered substituted or unsubstituted carbocycle or heterocycle;
each R⁴ is independently selected from the group consisting of H, halo, CN, CF₃, C₁₋₅ alkyl, C₃₋₇ cycloalkyl and heterocyclic;
R⁵ and R⁶ are selected as follows:
each of R⁵ and R⁶ is independently selected from C₁-C₃ alkyl, C₁-C₃ alkoxy, C₂-C₃ alkenyl, C₂-C₃ alkynyl, OH, CN, where applicable optionally substituted with 1-5 groups selected from C₁-C₃ alkyl, C₁-C₃ alkoxy, C₂-C₃ alkenyl, C₂-C₃ alkynyl, halo, OH and CN;
one of R⁵ and R⁶ is CH₃, CH₂CH₃, OCH₃ or OCH₂CH₃ and the other is H;
one of R⁵ and R⁶ is CF₃, CFHCH₃, CH₂CFH₂, CF₂CH₃, CFHCF₂H, CH₂CF₂H, CH₂CF₃, CFHCFH₂ or CF₂CF₃ and the other is H;
one of R⁵ and R⁶ is CH═CH₂, CF═CH₂, CH═CFH, CH═CF₂, CF═CFH or CF═CF₂ and the other is H;
one of R⁵ and R⁶ is C≡CH, C≡CF or CN and the other is H;
one of R⁵ and R⁶ is a substituted or unsubstituted cyclopropyl, cyclobutyl or cyclopentyl ring and the other is H; or
R⁵ and R⁶, together with the carbon atom to which they are attached to, are linked to form a substituted or unsubstituted 3- to 6-membered carbocyclic or heterocyclic ring;
each of R and R′ is independently H, or C₁₋₆ alkyl or cycloalkyl, optionally, R and R′, together with the nitrogen or carbon atom to which they are attached, form a 3- to 6-membered ring, each optionally substituted with 0-3 substituents independently selected from the group consisting of C₁₋₃ alkyl, halo, OH, OC₁₋₃ alkyl, and CN;
m is 0, 1, 2, 3 or 4;
n is 0, 1, 2, 3, 4 or 5; and
i is 0, 1 or 2.

2. The compound of claim 1, wherein Ring B is a substituted or unsubstituted 6-membered aryl.

3. The compound of claim 2, wherein Ring B is a substituted or unsubstituted phenyl.

4. (canceled)

5. The compound of claim 1, wherein Ring B is a substituted or unsubstituted 6-membered heteroaryl.

6-8. (canceled)

9. The compound of claim 1, wherein Ring B is a substituted or unsubstituted 5-membered heteroaryl.

10-12. (canceled)

13. The compound of claim 1, wherein Ring B is a substituted or unsubstituted bi- or multi-cyclic carbocyclic.

14. (canceled)

15. The compound of claim 1, wherein Ring B is a substituted or unsubstituted bi- or multi-cyclic heterocyclic.

16. (canceled)

17. The compound of claim 1, wherein X is N.

18. The compound of claim 1, wherein X is CH.

19. (canceled)

20. The compound of claim 1, wherein Ring A is: 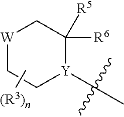
wherein
W is NR⁷, CR⁷R^{7′}, O or C(O); and
R⁷ and R^{7′} is independently selected from the group consisting of H, halo, CF₃, CH₂CF₃, CN, OH, C₁₋₅ alkyl, C₁₋₃ alkoxy, C₃₋₇ cycloalkyl, and 5- to 7-membered aryl or heteroaryl, optionally substituted with 1-3 groups selected from halo, CF₃, CH₂CF₃, CN, OH, C₁₋₅ alkyl, and C₁₋₃ alkoxy; or R⁷ and R^{7′}, together with the carbon and/or nitrogen atom(s) to which they are attached, form a 3- to 6-membered substituted or unsubstituted carbocycle or heterocycle.

21-22. (canceled)

23. The compound of claim 20, wherein Ring A is:

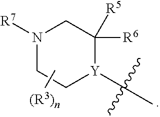

24-31. (canceled)

32. The compound of claim 20, wherein Ring A is: 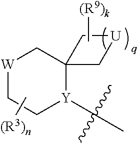
wherein
each U is independently selected from CH₂ and O; and
q is 0, 1, 2 or 3.

33-34. (canceled)

35. The compound of claim 23, wherein R⁷ is: 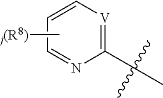
wherein
V is N or CH, optionally substituted with a halo or C₁-C₃ alkyl;
R⁸ is halo, CF₃, CH₂CF₃, CN, OH, C₁₋₅ alkyl, or C₃₋₇ cycloalkyl; and
j is 0, 1 or 2.

36-42. (canceled)

43. The compound of claim 1, wherein Y is N.

44. The compound of claim 1, wherein Y is CH.

45. The compound of claim 1, wherein Z is O.

46. (canceled)

47. The compound of claim 1, wherein R¹ is S(O)₂CHRR′.

48-74. (canceled)

75. A pharmaceutical composition comprising a compound according to claim 1.

76-78. (canceled)

79. A method for treating or reducing a disease or disorder, comprising administering to a subject in need thereof a therapeutically effective amount of a compound of claim 1.

80-84. (canceled)

85. A method for treating or reducing the effect of aging comprising administering to a subject in need thereof a therapeutically effective amount of a compound of claim 1.

86-97. (canceled)
